(19) United States
(12) Patent Application Publication
(10) Pub. No.: US 2024/0173419 A1
(43) Pub. Date: May 30, 2024

(54) CDK2 DEGRADERS AND USES THEREOF

(71) Applicant: Kymera Therapeutics, Inc., Watertown, MA (US)

(72) Inventors: Philip COLLIER, Watertown, MA (US); Xiaozhang ZHENG, Watertown, MA (US); Xiao ZHU, Watertown, MA (US); Melissa FORD, Watertown, MA (US); Matthew M. WEISS, Watertown, MA (US); Robert AVERSA, Watertown, MA (US); Lewis Dale PENNINGTON, Watertown, MA (US); Eamon COMER, Watertown, MA (US); Kiran Vijayakumari MAHASENAN, Watertown, MA (US); Yi ZHANG, Watertown, MA (US)

(21) Appl. No.: 18/453,156

(22) Filed: Aug. 21, 2023

Related U.S. Application Data
(60) Provisional application No. 63/373,018, filed on Aug. 19, 2022.
(60) Provisional application No. 63/380,914, filed on Oct. 25, 2022.
(60) Provisional application No. 63/493,926, filed on Apr. 3, 2023.
(60) Provisional application No. 63/522,640, filed on Jun. 22, 2023.

(51) Int. Cl. A61K 47/55; A61K 47/54

(52) U.S. Cl. CPC A61K 47/55; A61K 47/545

(57) ABSTRACT

The present invention provides compounds, compositions thereof, and methods of using the same.

### CROSS-REFERENCE TO RELATED APPLICATIONS

This application claims the benefit of priority to U.S. Provisional Appl. No. 63/373,018, filed Aug. 19, 2022, U.S. Provisional Appl. No. 63/380,914, filed Oct. 25, 2022, U.S. Provisional Appl. No. 63/493,926, filed Apr. 3, 2023, and U.S. Provisional Appl. No. 63/522,640, filed Jun. 22, 2023, the contents of which is herein incorporated by reference.

### TECHNICAL FIELD OF THE INVENTION

The present invention relates to compounds and methods useful for the modulation of cyclin-dependent kinase 2 (“CDK2”) protein via ubiquitination and/or degradation by compounds according to the present invention. The invention also provides pharmaceutically acceptable compositions comprising compounds of the present invention and methods of using said compositions in the treatment of various disorders.

### BACKGROUND OF THE INVENTION

Ubiquitin-Proteasome Pathway (UPP) or Ubiquitin-Proteasome System (UPS) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s. See generally Li et al. (PLOS One, 2008, 3, 1487) titled “Genome-wide and functional annotation of human E3 ubiquitin ligases identifies MULAN, a mitochondrial E3 that regulates the organelle's dynamics and signaling.”; Berndsen et al. (Nat. Struct. Mol. Biol., 2014, 21, 301-307) titled “New insights into ubiquitin E3 ligase mechanism”; Deshaies et al. (Ann. Rev. Biochem., 2009, 78, 399-434) titled “RING domain E3 ubiquitin ligases.”; Spratt et al. (Biochem. 2014, 458, 421-437) titled “RBR E3 ubiquitin ligases: new structures, new insights, new questions.”; and Wang et al. (Nat. Rev. Cancer., 2014, 14, 233-347) titled “Roles of F-box proteins in cancer.”

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (Crews C, Chemistry & Biology, 2010, 17(6):551-555; Schnnekloth JS Jr., Chembiochem, 2005, 6(1):40-46).

An ongoing need exists in the art for effective treatments for disease, especially cancers. Cyclin-dependent kinases (CDKs) are a family of serine/threonine kinases. Heterodimerized with regulatory subunits known as cyclins, such as cyclin E1 (“CCNE1”), CDKs become fully activated and regulate key cellular processes including cell cycle progression and cell division. Uncontrolled proliferation is a hallmark of cancer cells. The deregulation of the CDK activity is associated with abnormal regulation of cell-cycle, and is detected in virtually all forms of human cancers. As such, small molecule therapeutic agents that leverage UPP mediated protein degradation to target cancer-associated proteins such as cyclin-dependent kinase 2 (“CDK2”) or CDK2 and CCNE1 protein hold promise as therapeutic agents. Accordingly, there remains a need to find compounds that are CDK2 or CDK2 and CCNE1 degraders useful as therapeutic agents.

### SUMMARY OF THE INVENTION

The present application relates novel bifunctional compounds, which function to recruit CDK2 or CDK2 and CCNE1 protein to E3 ubiquitin ligase for degradation, and methods of preparation and uses thereof. In particular, the present disclosure provides bifunctional compounds, which find utility as modulators of targeted ubiquitination of CDK2 or CDK2 and CCNE1, which is then degraded and/or otherwise inhibited by the bifunctional compounds as described herein. Also provided are monovalent compounds, which find utility as inducers of targeted ubiquitination of CDK2 or CDK2 and CCNE1, which are then degraded and/or otherwise inhibited by the monovalent compounds as described herein. An advantage of the compounds provided herein is that a broad range of pharmacological activities is possible, consistent with the degradation/inhibition of CDK2 or CDK2 and CCNE1. In addition, the description provides methods of using an effective amount of the compounds as described herein for the treatment or amelioration of a disease condition, such those caused by aberrant CDK2 or CDK2 and CCNE1 activity.

The present application further relates to targeted degradation of CDK2 or CDK2 and CCNE1 protein through the use of bifunctional molecules, including bifunctional molecules that link a cereblon-binding moiety to a ligand that binds CDK2 or CDK2 and CCNE1.

It has now been found that compounds of this invention, and pharmaceutically acceptable compositions thereof, are effective as degraders of CDK2 or CDK2 and CCNE1 protein. Such compounds have the general formula I:

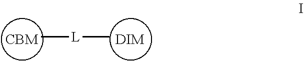

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of CDK2 protein. Such diseases, disorders, or conditions include those described herein.

Compounds provided by this invention are also useful for the study of CDK2 protein in biological and pathological phenomena; and the comparative evaluation of new CDK2 inhibitors or CDK2 degraders, in vitro or in vivo.

### DETAILED DESCRIPTION OF CERTAIN EMBODIMENTS

### 1. General Description of Certain Embodiments of the Invention

Compounds of the present invention, and compositions thereof, are useful as degraders and/or inhibitors of CDK protein. In some embodiments, a provided compound degrades and/or inhibits CDK2 protein. In some embodiments, a provided compound degrades and/or inhibits CDK2 and CCNE1 protein.

In certain embodiments, the present invention provides a compound of formula I:

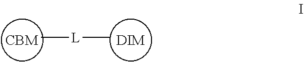

or a pharmaceutically acceptable salt thereof, wherein:
CBM is a CDK binding moiety capable of binding CDK2 or CDK2 and CCNE1;
L is a bivalent moiety that connects CBM to DIM; and
DIM is a degradation inducing moiety, such as a ligase binding moiety (LBM), lysine mimetic, or hydrogen atom.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic, bicyclic, bridged bicyclic, or spirocyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

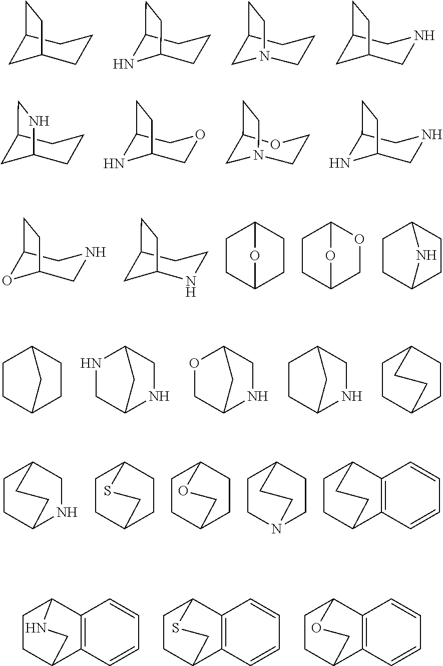

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR* (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

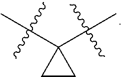 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. A heteroaryl ring may include one or more oxo (═O) or thioxo (═S) substituent. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic, bridged bicyclic, or spirocyclic. A heterocyclic ring may include one or more oxo (═O) or thioxo (═S) substituent. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted” means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —(CH₂)₀₋₄P(O)₂R^{∘}; —(CH₂)₀₋₄P(O)R^{∘}₂; —(CH₂)_{0- 4}P(O)(OR^{∘})₂; —(CH₂)₀₋₄OP(O)R^{∘}₂; —(CH₂)₀₋₄OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}₃, —OSiR^{•}₃, —C(O)SR^{•}, —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂)₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁-alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate. In some embodiments, the provided compounds are purified in salt form for convenience and/or ease of purification, e.g., using an acidic or basic mobile phase during chromatography. Salts forms of the provided compounds formed during chromotagraphic purification are contemplated herein and are readily apparent to those having skill in the art.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention

As used herein, the term “provided compound” refers to any genus, subgenus, and/or species set forth herein.

As used herein, the term “inhibitor” is defined as a compound that binds to and/or inhibits CDK2 or CDK2 and CCNE1 with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “degrader” is defined as a heterobifunctional compound that binds to and/or inhibits both CDK2 or CDK2 and CCNE1, and an E3 ligase with measurable affinity resulting in the ubiquitination and subsequent degradation of the CDK2 or CDK2 and CCNE1. In certain embodiments, a degrader has an DC₅₀ of less than about 50 μM, less than about 1 μM, less than about 500 nM, less than about 100 nM, less than about 10 nM, or less than about 1 nM. As used herein, the term “monovalent” refers to a degrader compound without an appended E3 ligase binding moiety.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41:2596-9 and Sun et al., Bioconjugate Chem., 2006, 17:52-7.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in CDK2 or CDK2 and CCNE1 activity between a sample comprising a compound of the present invention, or composition thereof, and CDK2 or CDK2 and CCNE1, and an equivalent sample comprising CDK2 or CDK2 and CCNE1, in the absence of said compound, or composition thereof.

### 3. Description of Exemplary Embodiments

As described above, in certain embodiments, the present invention provides a compound of formula I:

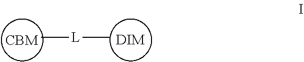

or a pharmaceutically acceptable salt thereof, wherein:
CBM is a CDK binding moiety capable of binding CDK2 or CDK2 and CCNE1;
L is a bivalent moiety that connects CBM to DIM; and
DIM is a degradation inducing moiety, such as a ligase binding moiety (LBM), lysine mimetic, or hydrogen atom.

CDK2 Binding Moiety (CBM)

As defined herein and described above, CBM is a CDK binding moiety capable of binding CDK2 protein. In some embodiments, CBM binds to CDK2 protein which then undergoes ubiquitination thereby marking the CDK2 for degradation via the Ubiquitin-Proteasome Pathway (UPP). In some embodiments, CBM is a CDK binding moiety capable of selectively binding and degrading CDK2 over other CDK proteins (e.g., CDK1, CDK3, CDK4, CDK5, CDK6, CDK7, CDK8, CDK9, etc.). In some embodiments, CBM is a CDK binding moiety capable of selectively binding and degrading CDK2 over one or more of CDK1, CDK4, and CDK9 proteins.

In some embodiments, CBM binds to CDK2 and CCNE1 protein which then undergoes ubiquitination thereby marking the CDK2 and CCNE1 for degradation via the Ubiquitin-Proteasome Pathway (UPP). In some embodiments, a provided compound is a dual CDK2 and CCNE1 degrader.

As defined herein and described below, wherein a formula is depicted using square brackets, e.g.,

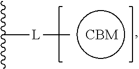

L is attached to a modifiable carbon, oxygen, nitrogen or sulfur atom within CBM including substitution or replacement of a defined group in CBM.

In certain embodiments, the present invention provides a compound of formula I, wherein CBM is a CDK2 or CDK2 and CCNE1 binding moiety thereby forming a compound of formula I-a:

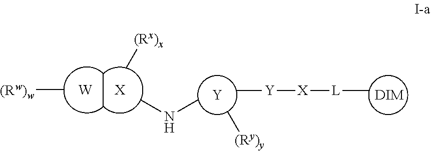

or a pharmaceutically acceptable salt thereof, wherein L and DIM are as defined above and described herein, and wherein:
Ring W and Ring X are independently fused rings selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur; Ring Y is a ring selected from phenylenyl, a 4 to 7-membered saturated or partially unsaturated
carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
Y is a covalent bond, —S(O)₂—, —S(O)—, —S(O)(NR)—, —P(O)R—, or —P(O)OR—;
X is —CR₂—, —CFR—, —CF₂—, —NR—, or an optionally substituted ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
each R^{w}, R^{x}, and R^{y} is independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂; or
   two R^{w} groups attached to the same carbon atom are optionally taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
w, x, and y are independently 0, 1, 2, 3, or 4.

In certain embodiments, the present invention provides a compound of formula I, wherein CBM is a CDK2 or CDK2 and CCNE1 binding moiety thereby forming a compound of formula I-b:

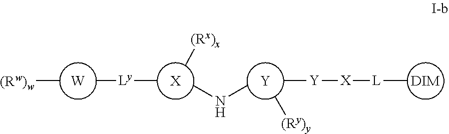

or a pharmaceutically acceptable salt thereof, wherein L and DIM are as defined above and described herein, and wherein:
Ring W and Ring X are independently rings selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Ring Y is a ring selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Y is a covalent bond, —S(O)₂—, —S(O)—, —S(O)(NR)—, —P(O)R—, or —P(O)OR—;
X is —CR₂—, —CFR—, —CF₂—, —NR—, or an optionally substituted ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
each R^{w}, R^{x}, and R^{y} is independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂; or
   two R^{w} groups attached to the same carbon atom are optionally taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
L^{y} is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CF₂—, —CRF—, —NR—, —S—, —S(O)—, —S(O)₂— or —CR═CR—; and
v is 0 or 1; and
w, x, and y are independently 0, 1, 2, 3, or 4.

In certain embodiments, the present invention provides a compound of formula I, wherein CBM is a CDK2 or CDK2 and CCNE1 binding moiety thereby forming a compound of formula I-a′:

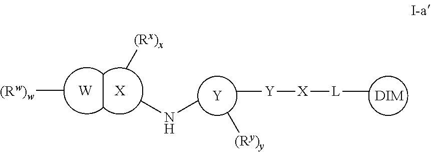

or a pharmaceutically acceptable salt thereof, wherein L and DIM are as defined above and described herein, and wherein:
Ring W and Ring X are independently fused rings selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Ring Y is a ring selected from phenylenyl, a 4 to 7-membered saturated or partially unsaturated carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
Y is a covalent bond, —S(O)₂—, —S(O)—, —S(O)(NR)—, —P(O)R—, —P(O)OR—, or

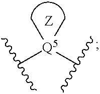

Ring Z is an optionally substituted 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur;
Q⁵ is carbon or sulfur;
X is —CR₂—, —CFR—, —CF₂—, —NR—, or an optionally substituted ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
each R^{w}, R^{x}, and R^{y} is independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂; or
   two R^{w} groups attached to the same or adjacent carbon atom are optionally taken together to form a spiro fused or 1,2-fused ring selected from a 3-12 membered saturated or partially unsaturated carbocyclyl and a 3-12 membered saturated or partially unsaturated heterocyclyl having 1-3 heteroatoms independently selected from nitrogen, oxygen, and sulfur;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form a 4-7 membered saturated, partially unsaturated, or heteroaryl ring having 0-3 heteroatoms, in addition to the atom or atoms to which they are attached, independently selected from nitrogen, oxygen, and sulfur; and
w, x, and y are independently 0, 1, 2, 3, or 4.

In certain embodiments, the present invention provides a compound of formula I, wherein CBM is a CDK2 or CDK2 and CCNE1 binding moiety thereby forming a compound of formula I-b′:

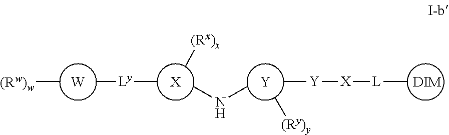

or a pharmaceutically acceptable salt thereof, wherein L and DIM are as defined above and described herein, and wherein:
Ring W and Ring X are independently rings selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Ring Y is a ring selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Y is a covalent bond, —S(O)₂—, —S(O)—, —S(O)(NR)—, —P(O)R—, —P(O)OR—, or

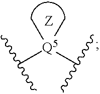

Ring Z is an optionally substituted 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur;
Q⁵ is carbon or sulfur;
X is —CR₂—, —CFR—, —CF₂—, —NR—, or an optionally substituted ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
each R^{w}, R^{x}, and R^{y} is independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂; or
   two R^{w} groups attached to the same or adjacent carbon atoms are optionally taken together to form a spiro fused or 1,2-fused ring selected from a 3-12 membered saturated or partially unsaturated carbocyclyl and a 3-12 membered saturated or partially unsaturated heterocyclyl having 1-3 heteroatoms independently selected from nitrogen, oxygen, and sulfur;
each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same or adjacent atom are optionally taken together with their intervening atoms to form a 4-7 membered saturated, partially unsaturated, or heteroaryl ring having 0-3 heteroatoms, in addition to the atom or atoms to which they are attached, independently selected from nitrogen, oxygen, and sulfur; and
L^{y} is a covalent bond, a C₁₋₃ bivalent straight or branched saturated or unsaturated hydrocarbon chain wherein 1-2 methylene units of the chain are independently and optionally replaced with —O—, —C(O)—, —C(S)—, —CF₂—, —CRF—, —NR—, —S—, —S(O)—, —S(O)₂— or —CR═CR—, or:
   L^{y} and one R^{x} are optionally taken together with their intervening atoms to form a 5-6 membered saturated, partially unsaturated or heteroaryl ring having 0-3 heteroatoms independently selected from oxygen, nitrogen or sulfur; and
v is 0 or 1; and
w, x, and y are independently 0, 1, 2, 3, or 4.

In certain embodiments, the present invention provides a compound of formula I-b or I-b′, wherein CBM is a CDK2 or CDK2 and CCNE1 binding moiety thereby forming a compound of formula 1-b-1:

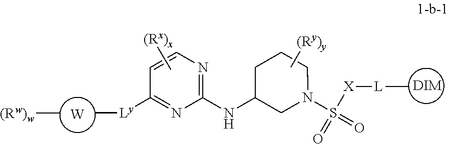

or a pharmaceutically acceptable salt thereof, wherein each of R^{x}, R^{y}, R^{w}, L^{y}, W, X, L, x, and w is as defined above and described in embodiments herein, both singly and in combination.

In some embodiments, Ring W is a 4 to 7-membered saturated or partially unsaturated carbocyclyl. In some embodiments, Ring W is a 4 to 7-membered saturated or partially unsaturated heterocyclyl with 1-3 heteroatoms independently selected from nitrogen, oxygen, and sulfur. In some embodiments, Ring W is a fused 4 to 7-membered saturated or partially unsaturated heterocyclyl with 1-3 heteroatoms independently selected from nitrogen, oxygen, and sulfur.

In certain embodiments, the present invention provides a compound of formula I-b or I-b′, wherein CBM is a CDK2 or CDK2 and CCNE1 binding moiety thereby forming a compound of formula 1-b-2:

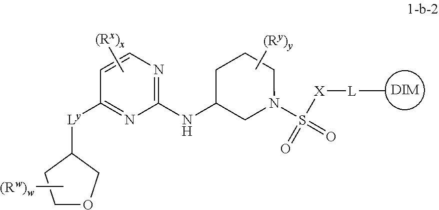

or a pharmaceutically acceptable salt thereof, wherein each of R^{x}, R^{y}, R^{w}, L, W, X, L, x, and w is as defined above and described in embodiments herein, both singly and in combination.

In certain embodiments, the present invention provides a compound of formula I-b or I-b′, wherein CBM is a CDK2 or CDK2 and CCNE1 binding moiety thereby forming a compound of formula 1-b-3:

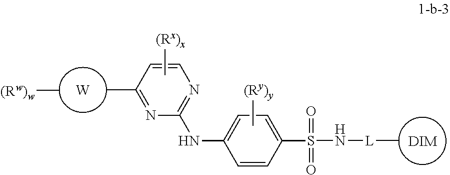

or a pharmaceutically acceptable salt thereof, wherein each of R^{x}, R^{y}, R^{w}, W, X, L, x, and w is as defined above and described in embodiments herein, both singly and in combination.

In some embodiments, Ring W is a 4 to 7-membered saturated or partially unsaturated carbocyclyl. In some embodiments, Ring W is a 4 to 7-membered saturated or partially unsaturated heterocyclyl with 1-3 heteroatoms independently selected from nitrogen, oxygen, and sulfur. In some embodiments, Ring W is a 5 to 6-membered saturated or partially unsaturated heterocyclyl with 1-3 heteroatoms independently selected from nitrogen, oxygen, and sulfur. In some embodiments, Ring W is a fused 4 to 7-membered saturated or partially unsaturated heterocyclyl with 1-3 heteroatoms independently selected from nitrogen, oxygen, and sulfur.

In certain embodiments, the present invention provides a compound of formula I-b or I-b′, wherein CBM is a CDK2 or CDK2 and CCNE1 binding moiety thereby forming a compound of formula 1-b-4, 1-b-5, or 1-b-6:

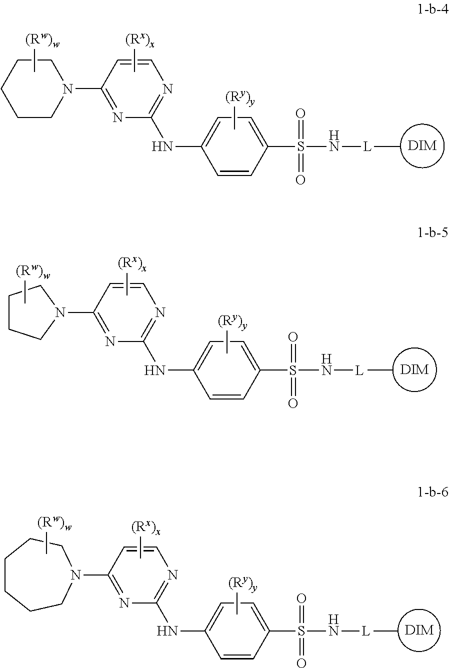

or a pharmaceutically acceptable salt thereof, wherein each of R^{x}, R^{y}, R^{w}, W, X, L, x, and w is as defined above and described in embodiments herein, both singly and in combination.

In certain embodiments, the present invention provides a compound of formula I-b or I-b′, wherein CBM is a CDK2 or CDK2 and CCNE1 binding moiety thereby forming a compound of formula 1-b-7:

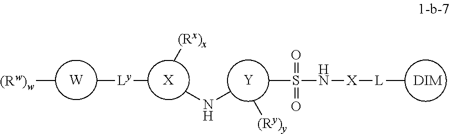

or a pharmaceutically acceptable salt thereof, wherein each of Ring X, Ring Y, Ring W, R^{x}, R^{y}, R^{w}, L, x, y, and w is as defined above and described in embodiments herein, both singly and in combination; and wherein X is an optionally substituted ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur.

In some embodiments, X is an optionally substituted 3 to 12-membered saturated or partially unsaturated bicyclic carbocyclylenyl or heterocyclylenyl with 1-3 heteroatoms independently selected from nitrogen, oxygen. In some embodiments, X is an optionally substituted 3 to 12-membered saturated or partially unsaturated bridged bicyclic carbocyclylenyl or heterocyclylenyl with 1-3 heteroatoms independently selected from nitrogen, oxygen. In some embodiments, X is an optionally substituted 3 to 12-membered saturated or partially unsaturated spirocyclic carbocyclylenyl or heterocyclylenyl with 1-3 heteroatoms independently selected from nitrogen, oxygen. In some embodiments, X is an optionally substituted 3 to 12-membered saturated or partially unsaturated spirocyclic carbocyclylenyl. In some embodiments, X is an optionally substituted 3 to 12-membered saturated or partially unsaturated spirocyclic heterocyclylenyl with 1-3 heteroatoms independently selected from nitrogen, oxygen, and sulfur. In some embodiments, X is an optionally substituted 3 to 12-membered saturated or partially unsaturated spirocyclic carbocyclylenyl. In some embodiments, X is an optionally substituted 3 to 12-membered saturated or partially unsaturated spirocyclic heterocyclylenyl with 1-3 heteroatoms independently selected from nitrogen, oxygen, and sulfur.

In certain embodiments, the present invention provides a compound of formula I-b or I-b′, wherein CBM is a CDK2 or CDK2 and CCNE1 binding moiety thereby forming a compound of formula 1-bb-1,1-bb-2, or 1-bb-3.

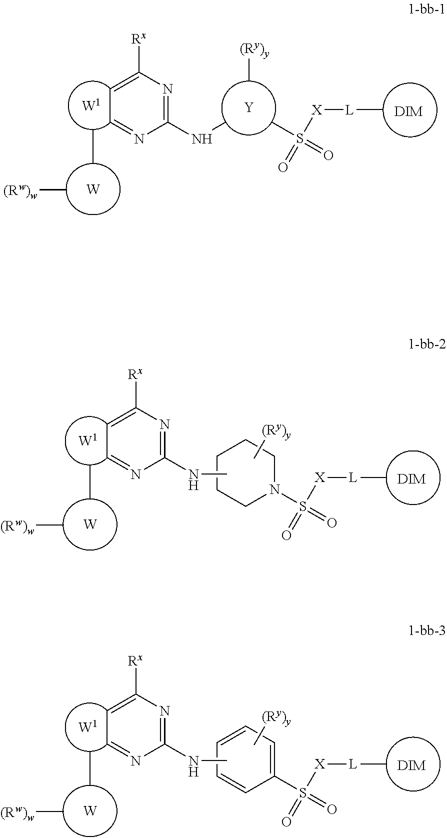

or a pharmaceutically acceptable salt thereof, wherein each of R, R^{Y}, R^{w}, W, X, L, x, and w, is as defined above and described in embodiments herein, both singly and in combination; and wherein L^{y} and one R^{x} are taken together with their intervening atoms to form Ring W¹, wherein Ring W¹ is a 5-6 membered saturated, partially unsaturated or heteroaryl ring having 0-3 heteroatoms independently selected from oxygen, nitrogen or sulfur.

In some embodiments, Ring W is a 4 to 7-membered saturated or partially unsaturated carbocyclyl. In some embodiments, Ring W is a 4 to 7-membered saturated or partially unsaturated heterocyclyl with 1-3 heteroatoms independently selected from nitrogen, oxygen, and sulfur. In some embodiments, Ring W is a 5 to 6-membered saturated or partially unsaturated heterocyclyl with 1-3 heteroatoms independently selected from nitrogen, oxygen, and sulfur. In some embodiments, Ring W is a fused 4 to 7-membered saturated or partially unsaturated heterocyclyl with 1-3 heteroatoms independently selected from nitrogen, oxygen, and sulfur.

In certain embodiments, the present invention provides a compound of formula I, wherein CBM is a CDK2 or CDK2 and CCNE1 binding moiety thereby forming a compound of formula I-d:

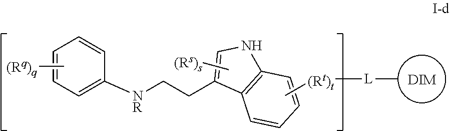

or a pharmaceutically acceptable salt thereof, wherein L and DIM are as defined above and described herein, and wherein:
each R^{w}, R^{s}, and R^{t} are independently selected from hydrogen, optionally substituted C₁₋₆ aliphatic, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
q, s, and t are independently 0, 1, 2, 3, or 4.

In certain embodiments, the present invention provides a compound of formula I-d, wherein CBM is a CDK2 or CDK2 and CCNE1 binding moiety thereby forming a compound of formula I-d-1:

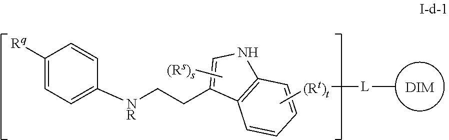

or a pharmaceutically acceptable salt thereof, wherein each of R_{q}, R^{s}, R^{t}, R, t, and s is as defined above and described in embodiments herein, both singly and in combination.

In certain embodiments, the present invention provides a compound of formula I-d, wherein CBM is a CDK2 or CDK2 and CCNE1 binding moiety thereby forming a compound of formula I-d-2:

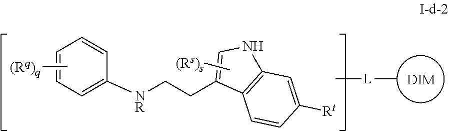

or a pharmaceutically acceptable salt thereof, wherein each of R^{q}, R^{s}, R^{t}, R, q, and s is as defined above and described in embodiments herein, both singly and in combination.

In certain embodiments, the present invention provides a compound of formula I-d, wherein CBM is a CDK2 or CDK2 and CCNE1 binding moiety thereby forming a compound of formula I-d-3:

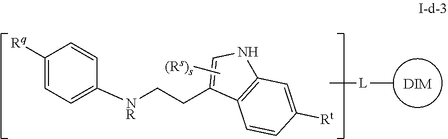

or a pharmaceutically acceptable salt thereof, wherein each of R^{q}, R^{s}, R^{t}, R, and s is as defined above and described in embodiments herein, both singly and in combination.

As defined generally above, Ring W, Ring X, and Ring Y are independently a ring selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.

In some embodiments, one or more of Ring W, Ring X, and Ring Y is a ring selected from phenyl. In some embodiments, one or more of Ring W, Ring X, and Ring Y is a 4 to 7-membered saturated or partially unsaturated carbocyclyl or heterocyclyl with 1-3 heteroatoms independently selected from nitrogen, oxygen, and sulfur. In some embodiments, one or more of Ring W, Ring X, and Ring Y is a 5 to 6-membered heteroaryl with 1-4 heteroatoms independently selected from nitrogen, oxygen and sulfur.

As defined generally above, Ring W and Ring X are independently fused rings selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.

In some embodiments, one or more of Ring W and Ring X is benzo. In some embodiments, one or more of Ring W and Ring X is a fused 4 to 7-membered saturated or partially unsaturated carbocyclyl. In some embodiments, one or more of Ring W and Ring X is a fused 4 to 7-membered saturated or partially unsaturated heterocyclyl with 1-3 heteroatoms independently selected from nitrogen, oxygen, and sulfur. In some embodiments, one or more of Ring W and Ring X is a fused 5 to 6-membered heteroaryl with 1-4 heteroatoms independently selected from nitrogen, oxygen and sulfur.

As defined generally above, Ring X is a bicyclic ring selected from naphthyl, a 9 to 10-membered saturated or partially unsaturated carbocyclyl or heterocyclyl with 1-4 heteroatoms independently selected from nitrogen, oxygen, and sulfur, and a 9 to 10-membered heteroaryl with 1-4 heteroatoms independently selected from nitrogen, oxygen and sulfur.

In some embodiments, Ring X is naphthyl. In some embodiments, Ring X is a 9 to 10-membered saturated or partially unsaturated carbocyclyl or heterocyclyl with 1-4 heteroatoms independently selected from nitrogen, oxygen, and sulfur. In some embodiments, Ring X is 9 to 10-membered heteroaryl with 1-4 heteroatoms independently selected from nitrogen, oxygen and sulfur. In some embodiments, Ring W is a fused 5 to 6-membered heteroaryl with 1-4 heteroatoms independently selected from nitrogen, oxygen and sulfur. In some embodiments, Ring W is a 4 to 7-membered saturated or partially unsaturated carbocyclyl. In some embodiments, Ring W is a 4 to 7-membered saturated or partially unsaturated heterocyclyl with 1-3 heteroatoms independently selected from nitrogen, oxygen, and sulfur. In some embodiments, Ring W is a fused 4 to 7-membered saturated or partially unsaturated heterocyclyl with 1-3 heteroatoms independently selected from nitrogen, oxygen, and sulfur. In some embodiments, Ring W is a fused 5 to 6-membered heteroaryl with 1-2 nitrogen. In some embodiments, Ring W is a 5 to 6-membered heteroaryl with 1-2 nitrogen. In some embodiments, Ring W is a fused 5 to 6-membered saturated or partially unsaturated heterocyclyl with 1-2 nitrogen. In some embodiments, Ring W is a 5 to 6-membered saturated or partially unsaturated heterocyclyl with 1-2 nitrogen. In some embodiments, Ring W is

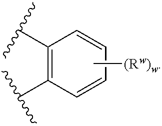

In some embodiments, Ring W is

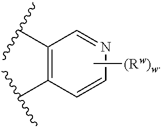

In some embodiments, Ring W is

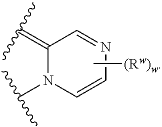

In some embodiments, Ring W is

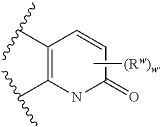

In some embodiments, Ring W is

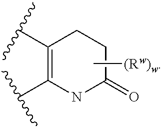

In some embodiments, Ring W is

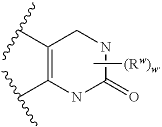

In some embodiments, Ring W is

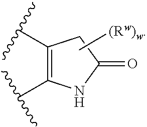

In some embodiments, Ring W is

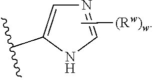

In some embodiments, Ring W is

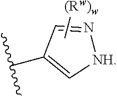

In some embodiments, Ring W is

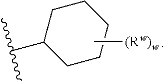

In some embodiments, Ring W is

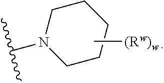

In some embodiments, Ring W is

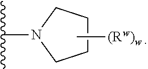

In some embodiments, Ring W is

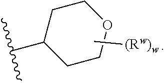

In some embodiments, Ring W is selected from those depicted in Table 1, below.

In some embodiments, Ring X is benzo. In some embodiments, Ring X is a fused 5 to 6-membered heteroaryl with 1-4 heteroatoms independently selected from nitrogen, oxygen and sulfur.

In some embodiments, Ring X is a fused 5 to 6-membered heteroaryl with 1-2 nitrogen. In some embodiments, Ring X is a fused 5 to 6-membered heteroaryl with 1 nitrogen. In some embodiments, Ring X is a fused 5-membered heteroaryl with sulfur or oxygen and optionally 1 nitrogen. In some embodiments, Ring X is

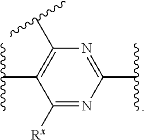

In some embodiments, Ring X is

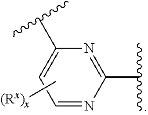

In some embodiments, Ring X is

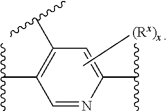

In some embodiments, Ring X is

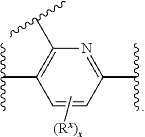

In some embodiments, Ring X is

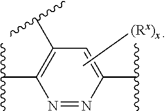

In some embodiments, Ring X is

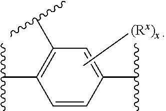

In some embodiments, Ring X is

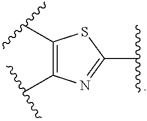 [SMILES: *c1nc(*)c(*)s1]

In some embodiments, Ring X is

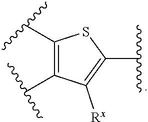

In some embodiments, Ring X is

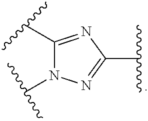 [SMILES: *c1nc(*)n(*)n1]

In some embodiments, Ring X is selected from those depicted in Table 1, below.

As defined generally above, Ring Y is a ring selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur.

In some embodiments, Ring Y is phenyl. In some embodiments, Ring Y is a 4 to 7-membered saturated or partially unsaturated carbocyclyl

In some embodiments, Ring Y is

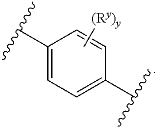

In some embodiments, Ring Y is

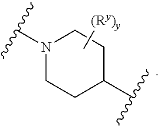

In some embodiments, Ring Y is

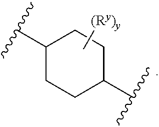

In some embodiments, Ring Y is selected from those depicted in Table 1, below. In some embodiments, Ring W, Ring X, and Ring Y are selected from those depicted in Table 1, below.

As defined generally above, Y is a covalent bond, —S(O)₂—, —S(O)—, —S(O)(NR)—, —P(O)R—, —P(O)OR—, or

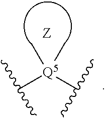

In some embodiments, Y is a covalent bond, —S(O)₂—, —S(O)—, —S(O)(NR)—, —P(O)R—, or —P(O)OR—.

In some embodiments, Y is —S(O)₂—, —S(O)—, —S(O)(NR)—, —P(O)R—, or —P(O)OR—.

In some embodiments, Y is a covalent bond. In some embodiments, Y is —S(O)₂—. In some embodiments, Y is —S(O)—. In some embodiments, Y is —S(O)(NR)—

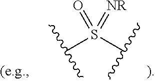

In some embodiments, Y is —P(O)R—. In some embodiments, Y is —P(O)OR—. In some embodiments, Y is —S(NR)₂—. In some embodiments, Y is —S(O)₂NR—.

In some embodiments, Y is —S(O)₁₋₂—. In some embodiments, Y is —S(O)(NH)—. In some embodiments, Y is —P(O)Me-.

In some embodiments, Y is

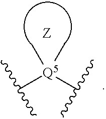

In some embodiments, Y is

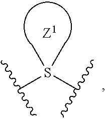 [SMILES: *S(*)(C)C]

wherein Ring Z¹ is an optionally substituted 3-12 membered saturated or partially unsaturated monocyclic, bicyclic, bridged bicyclic or spirocyclic heterocyclyl with an additional 0-2 heteroatoms independently selected from nitrogen, oxygen, and sulfur.

In some embodiments, Y is

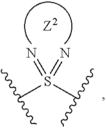 [SMILES: *S(*)(=N)=N]

wherein Ring Z² is an optionally substituted 3-12 membered saturated or partially unsaturated monocyclic, bicyclic, bridged bicyclic or spirocyclic heterocyclyl.

In some embodiments, Y is —S(NR)₂.

In some embodiments, Y is

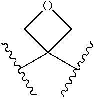 [SMILES: *C1(*)COC1]

In some embodiments, Y is selected from those depicted in Table 1, below.

As defined generally above, Ring Z is an optionally substituted 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur.

In some embodiments, Ring Z is an optionally substituted 3-12 membered saturated or partially unsaturated monocyclic, bicyclic, or spirocyclic carbocyclyl. In some embodiments, Ring Z is an optionally substituted 3-12 membered saturated or partially unsaturated monocyclic, bicyclic, or spirocyclic heterocyclyl with 1-3 heteroatoms independently selected from nitrogen, oxygen, and sulfur. In some embodiments, Ring Z is an optionally substituted 3-12 membered saturated or partially unsaturated monocyclic carbocyclyl. In some embodiments, Ring Z is an optionally substituted 3-12 membered saturated or partially unsaturated bicyclic carbocyclyl. In some embodiments, Ring Z is an optionally substituted 3-12 membered saturated or partially unsaturated bicyclic carbocyclyl. In some embodiments, Ring Z is an optionally substituted 3-12 membered saturated or partially unsaturated spirocyclic carbocyclyl. In some embodiments, Ring Z is an optionally substituted 3-12 membered saturated or partially unsaturated monocyclic heterocyclyl with 1-3 heteroatoms independently selected from nitrogen, oxygen, and sulfur. In some embodiments, Ring Z is an optionally substituted 3-12 membered saturated or partially unsaturated bicyclic heterocyclyl with 1-3 heteroatoms independently selected from nitrogen, oxygen, and sulfur. In some embodiments, Ring Z is an optionally substituted 3-12 membered saturated or partially unsaturated bicyclic heterocyclyl with 1-3 heteroatoms independently selected from nitrogen, oxygen, and sulfur. In some embodiments, Ring Z is an optionally substituted 3-12 membered saturated or partially unsaturated spirocyclic heterocyclyl with 1-3 heteroatoms independently selected from nitrogen, oxygen, and sulfur.

In some embodiments, Ring Z is selected from those depicted in Table 1, below.

As defined generally above, Q⁵ is carbon or sulfur.

In some embodiments, Q⁵ is carbon. In some embodiments, Q⁵ is sulfur.

In some embodiments, Q⁵ is selected from those depicted in Table 1, below.

As defined generally above, X is —CR₂—, —CFR—, —CF₂—, —NR—, or an optionally substituted ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur.

In some embodiments, X is —CR₂—. In some embodiments, X is —CH₂—. In some embodiments, X is —CHMe-. In some embodiments, X is —CMe₂-. In some embodiments, X is —CFR—. In some embodiments, X is —CF₂—. In some embodiments, X is —CH(OR)—. In some embodiments, X is —CMe(OR)—. In some embodiments, X is —CH(OMe)-. In some embodiments, X is —CMe(OH)—. In some embodiments, X is —CMe(CN)—. In some embodiments, X is —NR—. In some embodiments, X is —NH—. In some embodiments, X is —NMe-. In some embodiments, X is an optionally substituted phenylenyl. In some embodiments, X is an optionally substituted 3 to 12-membered saturated or partially unsaturated monocyclic, bicyclic, bridged bicyclic or spirocyclic carbocyclylenyl. In some embodiments, X is an optionally substituted monocyclic, bicyclic, bridged bicyclic or spirocyclic heterocyclylenyl with 1-3 heteroatoms independently selected from nitrogen, oxygen, and sulfur. In some embodiments, X is an optionally substituted 5 to 6-membered heteroarylenyl with 1-4 heteroatoms independently selected from nitrogen, oxygen and sulfur.

In some embodiments, X is an optionally substituted 3 to 12-membered saturated or partially unsaturated bicyclic carbocyclylenyl or heterocyclylenyl with 1-3 heteroatoms independently selected from nitrogen, oxygen. In some embodiments, X is an optionally substituted 3 to 12-membered saturated or partially unsaturated bridged bicyclic carbocyclylenyl or heterocyclylenyl with 1-3 heteroatoms independently selected from nitrogen, oxygen. In some embodiments, X is an optionally substituted 3 to 12-membered saturated or partially unsaturated spirocyclic carbocyclylenyl or heterocyclylenyl with 1-3 heteroatoms independently selected from nitrogen, oxygen. In some embodiments, X is an optionally substituted 3 to 12-membered saturated or partially unsaturated spirocyclic carbocyclylenyl. In some embodiments, X is an optionally substituted 3 to 12-membered saturated or partially unsaturated spirocyclic heterocyclylenyl with 1-3 heteroatoms independently selected from nitrogen, oxygen, and sulfur. In some embodiments, X is an optionally substituted 3 to 12-membered saturated or partially unsaturated spirocyclic carbocyclylenyl. In some embodiments, X is an optionally substituted 3 to 12-membered saturated or partially unsaturated spirocyclic heterocyclylenyl with 1-3 heteroatoms independently selected from nitrogen, oxygen, and sulfur. In some embodiments, X is an ortho-methyl piperdine. In some embodiments, X is an meta-fluoro piperdine. In some embodiments, X is an meta-methyl piperdine.

In some embodiments, X is

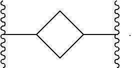 [SMILES: *C1CC(*)C1]

In some embodiments, X is

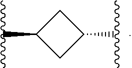 [SMILES: *[C@H]1C[C@H](*)C1]

In some embodiments, X is

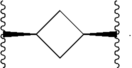 [SMILES: *[C@H]1C[C@@H](*)C1]

In some embodiments, X is

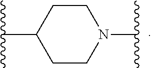 [SMILES: *C1CCN(*)CC1]

In some embodiments, X is

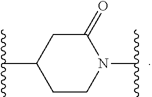 [SMILES: *C1CCN(*)C(=O)C1]

In some embodiments, X is

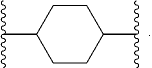 [SMILES: *C1CCC(*)CC1]

In some embodiments X is

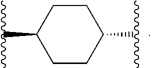 [SMILES: *[C@H]1CC[C@H](*)CC1]

In some embodiments, X is

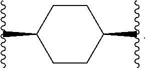 [SMILES: *[C@H]1CC[C@@H](*)CC1]

In some embodiments, X is

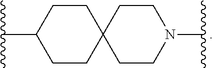 [SMILES: *C1CCC2(CC1)CCN(*)CC2]

In some embodiments, X is

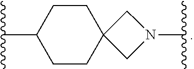 [SMILES: *C1CCC2(CC1)CN(*)C2]

In some embodiments, X is

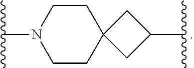 [SMILES: *C1CC2(CCN(*)CC2)C1]

In some embodiments, X is

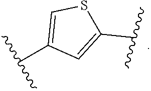 [SMILES: *c1csc(*)c1]

In some embodiments, X is

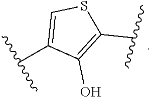 [SMILES: *c1csc(*)c1O]

In some embodiments, X is

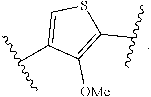 [SMILES: *c1csc(*)c1OC]

In some embodiments, X is

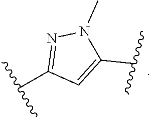 [SMILES: *c1cc(*)n(C)n1]

In some embodiments, X is

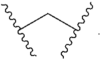 [SMILES: *C*]

In some embodiments, X is

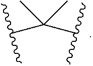 [SMILES: *C(*)(C)C]

In some embodiments, X is

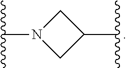 [SMILES: *C1CN(*)C1]

In some embodiments, X is

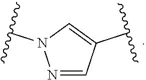 [SMILES: *c1cnn(*)c1]

In some embodiments, X is

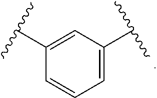 [SMILES: *c1cccc(*)c1]

In some embodiments, X is

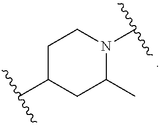 [SMILES: *C1CCN(*)C(C)C1]

In some embodiments, X is

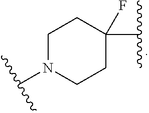 [SMILES: *N1CCC(*)(F)CC1]

In some embodiments, X is

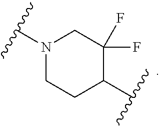 [SMILES: *C1CCN(*)CC1(F)F]

In some embodiments, X is

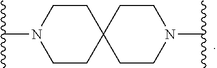 [SMILES: *N1CCC2(CC1)CCN(*)CC2]

In some embodiments, X is

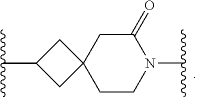 [SMILES: *C1CC2(CCN(*)C(=O)C2)C1]

In some embodiments, X is

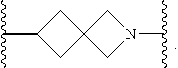 [SMILES: *C1CC2(C1)CN(*)C2]

In some embodiments, X is

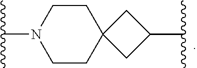 [SMILES: *C1CC2(CCN(*)CC2)C1]

In some embodiments, X is

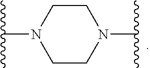 [SMILES: *N1CCN(*)CC1]

In some embodiments, X is

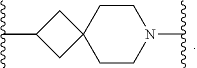 [SMILES: *C1CC2(CCN(*)CC2)C1]

In some embodiments, X is

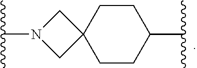 [SMILES: *C1CCC2(CC1)CN(*)C2]

In some embodiments, X is

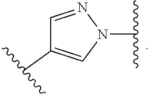 [SMILES: *c1cnn(*)c1]

In some embodiments, X is

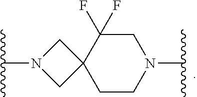 [SMILES: *N1CCC2(CN(*)C2)C(F)(F)C1]

In some embodiments, X is

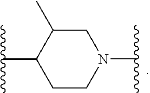 [SMILES: *C1CCN(*)CC1C]

In some embodiments, X is

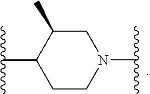 [SMILES: *C1CCN(*)C[C@@H]1C]

In some embodiments, X is

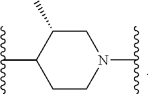 [SMILES: *C1CCN(*)C[C@H]1C]

In some embodiments, X is

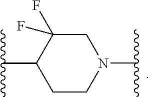 [SMILES: *C1CCN(*)CC1(F)F]

In some embodiments, X is

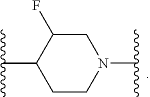 [SMILES: *C1CCN(*)CC1F]

In some embodiments, X is

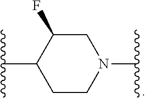 [SMILES: *C1CCN(*)C[C@@H]1F]

In some embodiments, X is

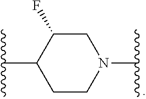 [SMILES: *C1CCN(*)C[C@H]1F]

In some embodiments, X is

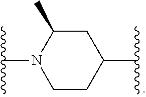 [SMILES: *C1CCN(*)[C@@H](C)C1]

In some embodiments, X is

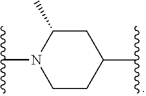 [SMILES: *C1CCN(*)[C@H](C)C1]

In some embodiments, Y connects to a carbon atom of X when X is an optionally substituted monocyclic, bicyclic, bridged bicyclic or spirocyclic heterocyclylenyl with 1-3 heteroatoms independently selected from nitrogen, oxygen, and sulfur, or when X is an optionally substituted 5 to 6-membered heteroarylenyl with 1-4 heteroatoms independently selected from nitrogen, oxygen and sulfur.

In some embodiments, X is

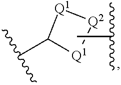

wherein each Q¹ is independently —O—, —S—, —C(O)—, —C(S)—, —CH₂—, —CHR—, —CR₂—, —NH—, or —NR—; and Q² is a C₁₋₉ bivalent saturated or unsaturated hydrocarbon chain or spirocyclic fused ring wherein 1-2 methylene units of the chain or ring are independently and optionally replaced with —O—, —S—, —C(O)—, —C(S)—, —CHR—, —CR₂—, —NH—, or —NR—.

In some embodiments, X is selected from those depicted in Table 1, below.

As defined generally above, each R^{w}, R^{x}, and R^{y} is independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂, or two R^{w} groups attached to the same carbon atom are optionally taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur.

In some embodiments, one or more of R^{w}, R^{x}, and R^{y} is hydrogen. In some embodiments, one or more of R^{w}, R^{x}, and R^{y} is R^{A}. In some embodiments, one or more of R^{w}, R^{x}, and R^{y} is halogen. In some embodiments, one or more of R^{w}, R^{x}, and R^{y} is —CN. In some embodiments, one or more of R^{w}, R^{x}, and R^{y} is —NO₂. In some embodiments, one or more of R^{w}, R^{x}, and R^{y} is —OR. In some embodiments, one or more of R^{w}, R^{x}, and R^{y} is —SR. In some embodiments, one or more of R^{w}, R^{x}, and R^{y} is —NR₂. In some embodiments, one or more of R^{w}, R^{x}, and R^{y} is —SiR₃. In some embodiments, one or more of R^{w}, R^{x}, and R is —S(O)₂R. In some embodiments, one or more of R^{w}, R^{x}, and R^{y} is —S(O)₂NR₂. In some embodiments, one or more of R^{w}, R^{x}, and R^{y} is —S(O)R. In some embodiments, one or more of R^{w}, R^{x}, and R^{y} is —C(O)R. In some embodiments, one or more of R^{w}, R^{x}, and R^{y} is —C(O)OR. In some embodiments, one or more of R^{w}, R^{x}, R^{y}, and R^{z} is —C(O)NR₂. In some embodiments, one or more of R^{w}, R^{x}, and R^{y} is —C(O)NROR. In some embodiments, one or more of R^{w}, R^{x}, and R^{y} is —OC(O)R. In some embodiments, one or more of R^{w}, R^{x}, and R^{y} is —OC(O)NR₂. In some embodiments, one or more of R^{w}, R^{x}, and R^{y} is —OP(O)R₂. In some embodiments, one or more of R^{w}, R^{x}, and R^{y} is —OP(O)(OR)₂. In some embodiments, one or more of R^{w}, R^{x}, and R is —OP(O)(OR)NR₂. In some embodiments, one or more of R^{w}, R^{x}, and R^{y} is —P(O)R₂. In some embodiments, one or more of R^{w}, R^{x}, and R^{y} is —P(O)(OR)₂. In some embodiments, one or more of R^{w}, R^{x}, and R^{y} is —P(O)(OR)NR₂. In some embodiments, one or more of R^{w}, R^{x}, and R^{y} is —P(O)(NR₂)₂—. In some embodiments, one or more of R^{w}, R^{x}, and R^{y} is —NRC(O)OR. In some embodiments, one or more of R^{w}, R^{x}, and R is —NRC(O)R. In some embodiments, one or more of R^{w}, R^{x}, and R^{y} is —NRC(O)N(R)₂. In some embodiments, one or more of R^{w}, R^{x}, and R^{y} is —NRS(O)₂R. In some embodiments, one or more of R^{w}, R^{x}, and R^{y} is —NP(O)R₂. In some embodiments, one or more of R^{w}, R^{x}, and R^{y} is —NRP(O)(OR)₂. In some embodiments, one or more of R^{w}, R^{x}, and R^{y} is —NRP(O)(OR)NR₂. In some embodiments, one or more of R^{w}, R^{x}, and R^{y} is —NRP(O)(NR₂)₂. In some embodiments, one or more of R^{w}, R^{x}, and R^{y} is —CF₃. In some embodiments, two R^{w} groups attached to the same carbon atom are taken together to form a 3-5 membered saturated or partially unsaturated carbocyclic spiro fused ring. In some embodiments, two R^{w} groups attached to the same carbon atom are optionally taken together to form a 3-5 membered saturated or partially unsaturated heterocyclic spiro fused ring having 1-2 heteroatoms independently selected from nitrogen, oxygen, and sulfur.

In some embodiments, one or more R^{w} is selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂, or two R^{w} groups attached to the same carbon atom are optionally taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur.

In some embodiments, one or more R^{w} is hydrogen. In some embodiments, one or more R^{w} is R^{A}. In some embodiments, one or more R^{w} is halogen. In some embodiments, one or more R^{w} is —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, or —NRP(O)(NR₂)₂. In some embodiments two R^{w} groups attached to the same carbon atom are taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur.

In some embodiments, two R^{w} groups attached to the same carbon atom are taken together to form a spiro fused 3-5 membered saturated or partially unsaturated carbocyclyl. In some embodiments, two R^{w} groups attached to the same carbon atom are taken together to form a spiro fused 3-5 membered saturated or partially unsaturated heterocyclyl having 1-2 heteroatoms independently selected from nitrogen, oxygen, and sulfur. In some embodiments, two R^{w} groups attached to the same or adjacent carbon atom are optionally taken together to form a spiro fused or 1,2-fused ring selected from a 3-12 membered saturated or partially unsaturated carbocyclyl and a 3-12 membered saturated or partially unsaturated heterocyclyl having 1-3 heteroatoms independently selected from nitrogen, oxygen, and sulfur. In some embodiments, two R^{w} groups attached to the same or adjacent carbon atom are taken together to form a spiro fused or 1,2-fused ring selected from a 3-12 membered saturated or partially unsaturated carbocyclyl and a 3-12 membered saturated or partially unsaturated heterocyclyl having 1-3 heteroatoms independently selected from nitrogen, oxygen, and sulfur. In some embodiments, two R^{w} groups attached to the same carbon atom are taken together to form a spiro fused 3-12 membered saturated or partially unsaturated carbocyclyl.

In some embodiments, two R^{w} groups attached to the same carbon atom are taken together to form a spiro fused 3-12 membered saturated or partially unsaturated heterocyclyl having 1-3 heteroatoms independently selected from nitrogen, oxygen, and sulfur. In some embodiments, two R^{w} groups attached to adjacent carbon atoms are taken together to form a 1,2-fused 3-12 membered saturated or partially unsaturated carbocyclyl. In some embodiments, two R^{w} groups attached to adjacent carbon atoms are taken together to form a 1,2-fused 3-12 membered saturated or partially unsaturated heterocyclyl having 1-3 heteroatoms independently selected from nitrogen, oxygen, and sulfur.

In some embodiments, R^{w} is fluoro. In some embodiments, R^{w} is chloro. In some embodiments, R^{w} is bromo. In some embodiments, R^{w} is —CN. In some embodiments, R^{w} is —OH. In some embodiments, R^{w} is —OMe. In some embodiments, R^{w} is -OiPr. In some embodiments, R^{w} is —O— cyclopropyl. In some embodiments, R^{w} is —O-cyclobutyl. In some embodiments, R^{w} is —CONH₂.

In some embodiments, R^{w} is R^{A}. In some embodiments, R^{w} is methyl. In some embodiments, R^{w} is ethyl. In some embodiments, R^{w} is isopropyl. In some embodiments, R^{w} is tert-butyl. In some embodiments, R^{w} is cyclopropyl. In some embodiments, R^{w} is cyclobutyl. In some embodiments, R^{w} is cyclopentyl. In some embodiments, R^{w} is —CHF₂. In some embodiments, R^{w} is —CF₃. In some embodiments, R^{w} is —CH₂CHF₂. In some embodiments, R^{w} is —CH(Me)CF₃. In some embodiments, R^{w} is —CMe₂OH. In some embodiments, R^{w} is

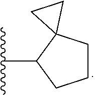 [SMILES: *C1CCCC12CC2]

In some embodiments, R^{w} is

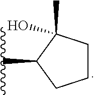 [SMILES: *[C@@H]1CCC[C@@]1(C)O]

In some embodiments, R^{w} is

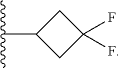 [SMILES: *C1CC(F)(F)C1]

In some embodiments, R^{w} is

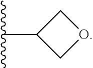 [SMILES: *C1COC1]

In some embodiments, R^{w} is

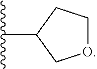 [SMILES: *C1CCOC1]

In some embodiments, R^{w} is

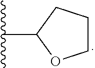 [SMILES: *C1CCCO1]

In some embodiments, R^{w} is

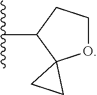 [SMILES: *C1CCOC12CC2]

In some embodiments, R^{w} is

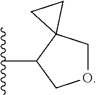 [SMILES: *C1COCC12CC2]

In some embodiments, R^{w} is

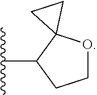 [SMILES: *C1CCOC12CC2]

In some embodiments, R^{w} is

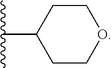 [SMILES: *C1CCOCC1]

In some embodiments, R^{w} is

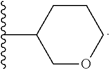 [SMILES: *C1CCCOC1]

In some embodiments, R^{w} is

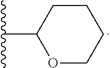 [SMILES: *C1CCCCO1]

In some embodiments, R^{w} is

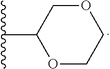 [SMILES: *C1COCCO1]

In some embodiments, R^{w} is

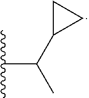 [SMILES: *C(C)C1CC1]

In some embodiments, R^{w} is

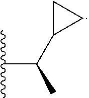 [SMILES: *[C@H](C)C1CC1]

In some embodiments, R^{w} is

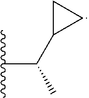 [SMILES: *[C@@H](C)C1CC1]

In some embodiments, R^{w} is

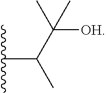 [SMILES: *C(C)C(C)(C)O]

In some embodiments, R^{w} is

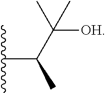 [SMILES: *[C@H](C)C(C)(C)O]

In some embodiments, R^{w} is

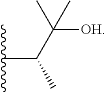 [SMILES: *[C@@H](C)C(C)(C)O]

In some embodiments, two R^{w} cyclize to form cyclopropylenyl. In some embodiments, two R^{w} cyclize to form an optionally substituted cyclobutylenyl. In some embodiments, two R^{w} cyclize to form cyclobutylenyl. In some embodiments, two R^{w} cyclize to form In some embodiments, two R^{w} cyclize to form

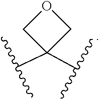 [SMILES: *C1(*)COC1]

In some embodiments, two R^{w} cyclize to form

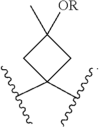

In some embodiments, two R^{w} cyclize to form

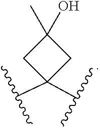 [SMILES: *C1(*)CC(C)(O)C1]

In some embodiments, one or more R^{X} is selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂.

In some embodiments, one or more R^{x} is hydrogen. In some embodiments, one or more R^{x} is R^{A}. In some embodiments, one or more R^{x} is halogen. In some embodiments, one or more R^{x} is —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, or —NRP(O)(NR₂)₂.

In some embodiments, R^{x} is bromo. In some embodiments, R^{x} is R^{A}. In some embodiments, R^{x} is —CF₃.

In some embodiments, one or more R^{y} is selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂.

In some embodiments, one or more R^{y} is hydrogen. In some embodiments, one or more R^{y} is R^{A}. In some embodiments, one or more R^{y} is halogen. In some embodiments, one or more R^{y} is —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —CR₂NRC(O)R, —CR₂NRC(O)NR₂, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, or —NRP(O)(NR₂)₂.

In some embodiments, R^{y} is R^{A}. In some embodiments, R^{y} is methyl.

In some embodiments, R^{w}, R^{x}, and R^{y} are selected from those depicted in Table 1, below.

As defined generally above, each R^{q}, R^{s}, and R^{t} are independently selected from hydrogen, optionally substituted C₁₋₆ aliphatic, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂.

In some embodiments, R⁹ is NO₂. In some embodiments, R⁹ is CF₃. In some embodiments, R⁹ is SF₅. In some embodiments, R⁹ is a halogen. In some embodiments, R⁹ is C1. In some embodiments, R⁹ is F. In some embodiments, R⁹ is Br. In some embodiments, R⁹ is CN. In some embodiments, R⁹ is OR.

In some embodiments, R is H. In some embodiments, R is a halogen. In some embodiments, R is Br. In some embodiments, R is CN.

In some embodiments, R^{s} is H. In some embodiments, R^{s} is Me.

In some embodiments, R^{q}, R^{s}, and R^{t} are selected from those depicted in Table 1, below.

As defined generally above, each R^{A} is independently an optionally substituted group selected from C₁₋₆ aliphatic, phenyl, a 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{A} is an optionally substituted C₁₋₆ aliphatic. In some embodiments, R^{A} is an optionally substituted phenyl. In some embodiments, R^{A} is an optionally substituted 3-12 membered saturated or partially unsaturated monocyclic, bicyclic, bridged bicyclic or spirocyclic carbocyclyl. In some embodiments, R^{A} is an optionally substituted 3-12 membered saturated or partially unsaturated monocyclic, bicyclic, bridged bicyclic or spirocyclic heterocyclyl with 1-3 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur.

In some embodiments, R^{A} is C₁₋₆alkyl (e.g., methyl, ethyl, isopropyl, etc.). In some embodiments, R^{A} is C₁₋₆haloalkyl (e.g., —CF₃, —CHF₂, etc.).

In some embodiments, R^{A} is selected from those depicted in Table 1, below.

As defined generally above, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted a 4-7 membered saturated or partially unsaturated heterocyclic having 1-2 heteroatoms independently selected from nitrogen, oxygen, and sulfur. In some embodiments, R is an optionally substituted a 5-6 membered heteroaryl ring having 1-4 heteroatoms independently selected from nitrogen, oxygen, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur.

In some embodiments, R is C₁₋₆alkyl (e.g., methyl, ethyl, isopropyl, etc.). In some embodiments, R is C₁₋₆haloalkyl (e.g., —CF₃, —CHF₂, etc.).

In some embodiments, R is selected from those depicted in Table 1, below.

As defined generally above, L is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CF₂—, —CRF—, —NR—, —S—, —S(O)—, —S(O)₂— or —CR═CR—.

In some embodiments, L is a covalent bond. In some embodiments, L is a C1-3 bivalent straight or branched saturated or unsaturated hydrocarbon chain wherein 1-2 methylene units of the chain are independently and optionally replaced with —O—, —C(O)—, —C(S)—, —CF₂—, —CRF—, —NR—, —S—, —S(O)—, —S(O)₂— or —CR═CR—.

In some embodiments, L is selected from those depicted in Table 1, below.

As defined generally above, w, x, and y are independently 0, 1, 2, 3, or 4.

In some embodiments, one or more of w, x, and y is 0. In some embodiments, one or more of w, x, and y is 1. In some embodiments, one or more of w, x, and y is 2. In some embodiments, one or more of w, x, and y is 3. In some embodiments, one or more of w, x, and y is 4.

In some embodiments, w is 0 or 1. In some embodiments, w is 1 or 2. In some embodiments, x is 0 or 1. In some embodiments, x is 1 or 2. In some embodiments, y is 0 or 1. In some embodiments, y is 1 or 2.

In some embodiments, w, x, and y are selected from those depicted in Table 1, below.

As defined generally above, q, s, and t are independently 0, 1, 2, 3, or 4.

In some embodiments, one or more of q, s, and tis 0. In some embodiments, one or more of q, s, and tis 1. In some embodiments, one or more of q, s, and t is 2. In some embodiments, one or more of q, s, and t is 3. In some embodiments, one or more of q, s, and t is 4.

In some embodiments, q, s, and t are selected from those depicted in Table 1, below.

In some embodiments, CBM is

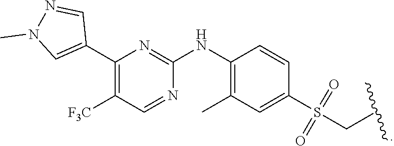

In some embodiments, CBM is

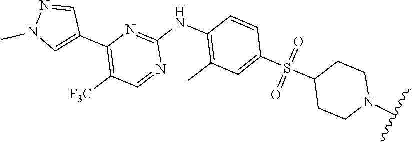 [SMILES: *N1CCC(S(=O)(=O)c2ccc(Nc3ncc(C(F)(F)F)c(-c4cnn(C)c4)n3)c(C)c2)CC1]

In some embodiments, CBM is

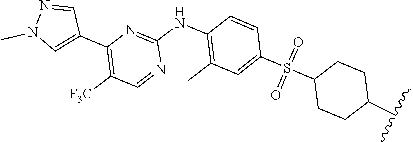 [SMILES: *C1CCC(S(=O)(=O)c2ccc(Nc3ncc(C(F)(F)F)c(-c4cnn(C)c4)n3)c(C)c2)CC1]

In some embodiments, CBM is

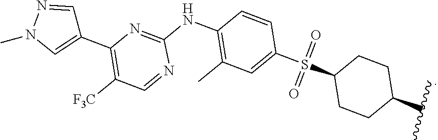 [SMILES: *[C@H]1CC[C@@H](S(=O)(=O)c2ccc(Nc3ncc(C(F)(F)F)c(-c4cnn(C)c4)n3)c(C)c2)CC1]

In some embodiments, CBM is

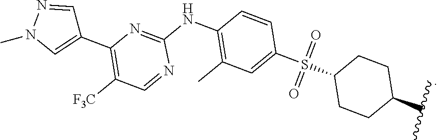 [SMILES: *[C@H]1CC[C@H](S(=O)(=O)c2ccc(Nc3ncc(C(F)(F)F)c(-c4cnn(C)c4)n3)c(C)c2)CC1]

In some embodiments, CBM is

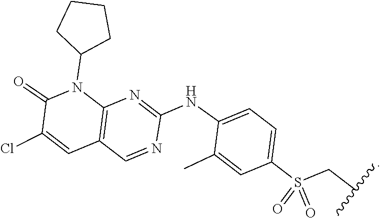 [SMILES: *CS(=O)(=O)c1ccc(Nc2ncc3cc(Cl)c(=O)n(C4CCCC4)c3n2)c(C)c1]

In some embodiments, CBM is

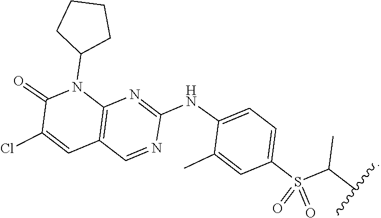 [SMILES: *C(C)S(=O)(=O)c1ccc(Nc2ncc3cc(Cl)c(=O)n(C4CCCC4)c3n2)c(C)c1]

In some embodiments, CBM is

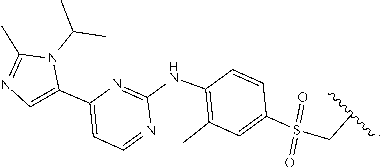 [SMILES: *CS(=O)(=O)c1ccc(Nc2nccc(-c3cnc(C)n3C(C)C)n2)c(C)c1]

In some embodiments, CBM is

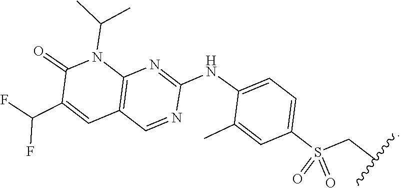 [SMILES: *CS(=O)(=O)c1ccc(Nc2ncc3cc(C(F)F)c(=O)n(C(C)C)c3n2)c(C)c1]

In some embodiments, CBM is

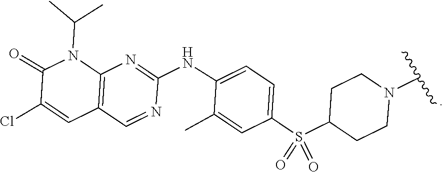 [SMILES: *N1CCC(S(=O)(=O)c2ccc(Nc3ncc4cc(Cl)c(=O)n(C(C)C)c4n3)c(C)c2)CC1]

In some embodiments, CBM is

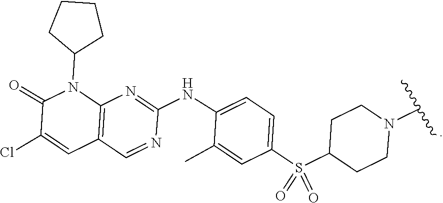 [SMILES: *N1CCC(S(=O)(=O)c2ccc(Nc3ncc4cc(Cl)c(=O)n(C5CCCC5)c4n3)c(C)c2)CC1]

In some embodiments, CBM is

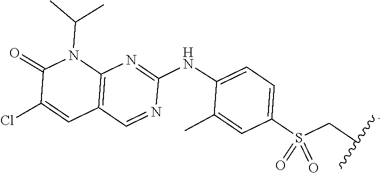 [SMILES: *CS(=O)(=O)c1ccc(Nc2ncc3cc(Cl)c(=O)n(C(C)C)c3n2)c(C)c1]

In some embodiments, CBM is

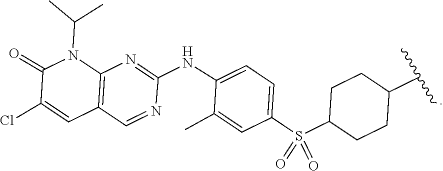 [SMILES: *C1CCC(S(=O)(=O)c2ccc(Nc3ncc4cc(Cl)c(=O)n(C(C)C)c4n3)c(C)c2)CC1]

In some embodiments, CBM is

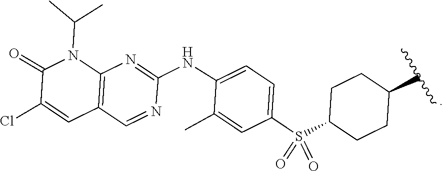 [SMILES: *[C@H]1CC[C@H](S(=O)(=O)c2ccc(Nc3ncc4cc(Cl)c(=O)n(C(C)C)c4n3)c(C)c2)CC1]

In some embodiments, CBM is

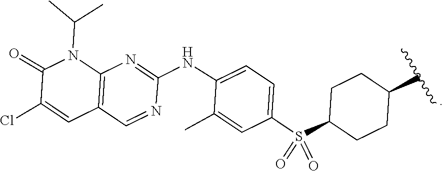 [SMILES: *[C@H]1CC[C@@H](S(=O)(=O)c2ccc(Nc3ncc4cc(Cl)c(=O)n(C(C)C)c4n3)c(C)c2)CC1]

In some embodiments, CBM is

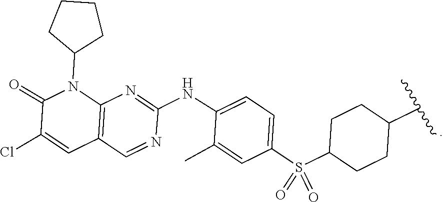 [SMILES: *C1CCC(S(=O)(=O)c2ccc(Nc3ncc4cc(Cl)c(=O)n(C5CCCC5)c4n3)c(C)c2)CC1]

In some embodiments, CBM is

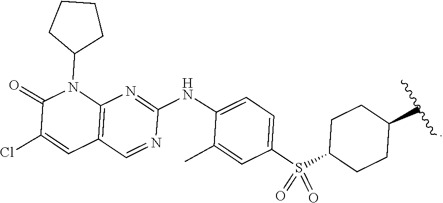 [SMILES: *[C@H]1CC[C@H](S(=O)(=O)c2ccc(Nc3ncc4cc(Cl)c(=O)n(C5CCCC5)c4n3)c(C)c2)CC1]

In some embodiments, CBM is

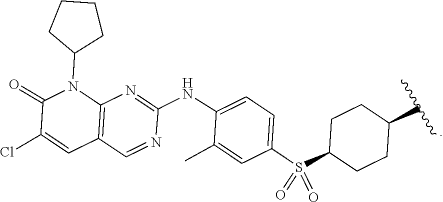 [SMILES: *[C@H]1CC[C@@H](S(=O)(=O)c2ccc(Nc3ncc4cc(Cl)c(=O)n(C5CCCC5)c4n3)c(C)c2)CC1]

In some embodiments, CBM is

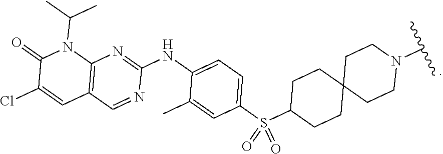 [SMILES: *N1CCC2(CCC(S(=O)(=O)c3ccc(Nc4ncc5cc(Cl)c(=O)n(C(C)C)c5n4)c(C)c3)CC2)CC1]

In some embodiments, CBM is

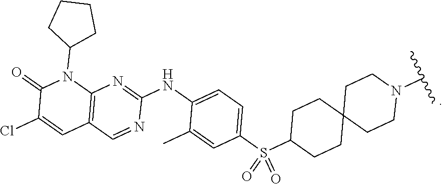 [SMILES: *N1CCC2(CCC(S(=O)(=O)c3ccc(Nc4ncc5cc(Cl)c(=O)n(C6CCCC6)c5n4)c(C)c3)CC2)CC1]

In some embodiments, CBM is

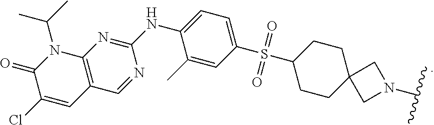 [SMILES: *N1CC2(CCC(S(=O)(=O)c3ccc(Nc4ncc5cc(Cl)c(=O)n(C(C)C)c5n4)c(C)c3)CC2)C1]

In some embodiments, CBM is

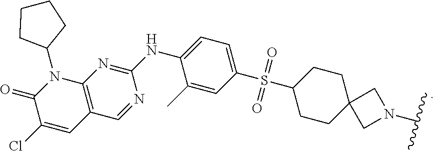 [SMILES: *N1CC2(CCC(S(=O)(=O)c3ccc(Nc4ncc5cc(Cl)c(=O)n(C6CCCC6)c5n4)c(C)c3)CC2)C1]

In some embodiments, CBM is

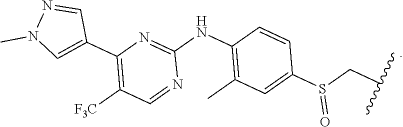 [SMILES: *CS(=O)c1ccc(Nc2ncc(C(F)(F)F)c(-c3cnn(C)c3)n2)c(C)c1]

In some embodiments, CBM is

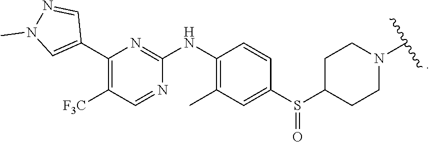 [SMILES: *N1CCC(S(=O)c2ccc(Nc3ncc(C(F)(F)F)c(-c4cnn(C)c4)n3)c(C)c2)CC1]

In some embodiments, CBM is

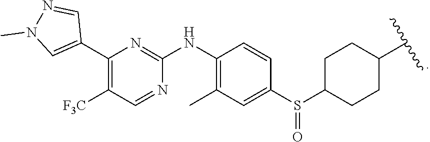 [SMILES: *C1CCC(S(=O)c2ccc(Nc3ncc(C(F)(F)F)c(-c4cnn(C)c4)n3)c(C)c2)CC1]

In some embodiments, CBM is

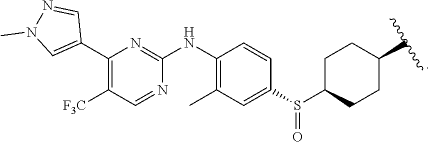 [SMILES: *[C@H]1CC[C@@H]([S@](=O)c2ccc(Nc3ncc(C(F)(F)F)c(-c4cnn(C)c4)n3)c(C)c2)CC1]

In some embodiments, CBM is

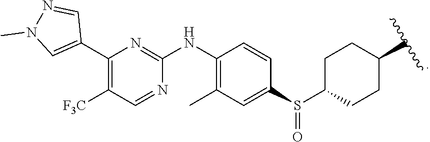 [SMILES: *[C@H]1CC[C@H]([S@@](=O)c2ccc(Nc3ncc(C(F)(F)F)c(-c4cnn(C)c4)n3)c(C)c2)CC1]

In some embodiments, CBM is

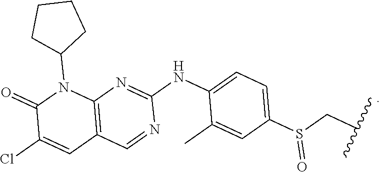 [SMILES: *CS(=O)c1ccc(Nc2ncc3cc(Cl)c(=O)n(C4CCCC4)c3n2)c(C)c1]

In some embodiments, CBM is

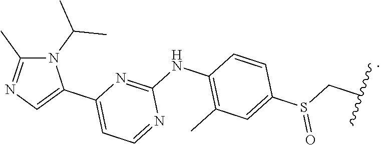 [SMILES: *CS(=O)c1ccc(Nc2nccc(-c3cnc(C)n3C(C)C)n2)c(C)c1]

In some embodiments, CBM is

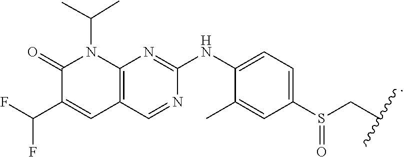 [SMILES: *CS(=O)c1ccc(Nc2ncc3cc(C(F)F)c(=O)n(C(C)C)c3n2)c(C)c1]

In some embodiments, CBM is

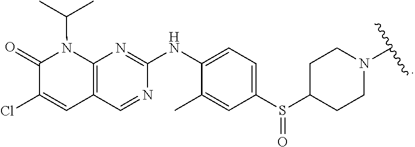 [SMILES: *N1CCC(S(=O)c2ccc(Nc3ncc4cc(Cl)c(=O)n(C(C)C)c4n3)c(C)c2)CC1]

In some embodiments, CBM is

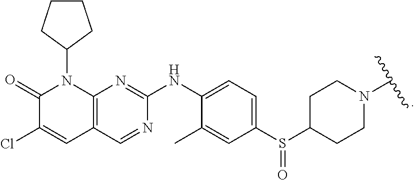 [SMILES: *N1CCC(S(=O)c2ccc(Nc3ncc4cc(Cl)c(=O)n(C5CCCC5)c4n3)c(C)c2)CC1]

In some embodiments, CBM is

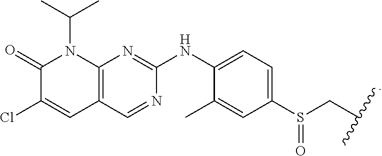 [SMILES: *CS(=O)c1ccc(Nc2ncc3cc(Cl)c(=O)n(C(C)C)c3n2)c(C)c1]

In some embodiments, CBM is

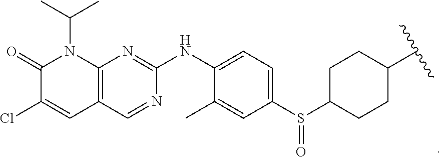 [SMILES: *C1CCC(S(=O)c2ccc(Nc3ncc4cc(Cl)c(=O)n(C(C)C)c4n3)c(C)c2)CC1]

In some embodiments, CBM is

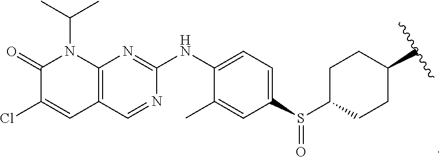 [SMILES: *[C@H]1CC[C@H]([S@@](=O)c2ccc(Nc3ncc4cc(Cl)c(=O)n(C(C)C)c4n3)c(C)c2)CC1]

In some embodiments, CBM is

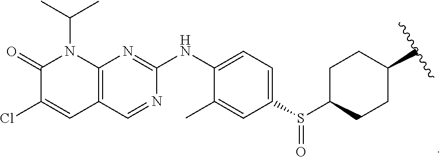 [SMILES: *[C@H]1CC[C@@H]([S@](=O)c2ccc(Nc3ncc4cc(Cl)c(=O)n(C(C)C)c4n3)c(C)c2)CC1]

In some embodiments, CBM is

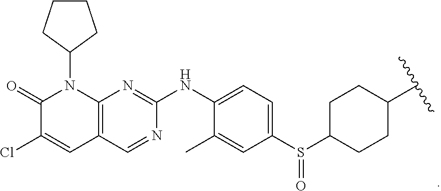 [SMILES: *C1CCC(S(=O)c2ccc(Nc3ncc4cc(Cl)c(=O)n(C5CCCC5)c4n3)c(C)c2)CC1]

In some embodiments, CBM is

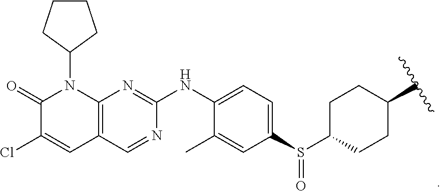 [SMILES: *[C@H]1CC[C@H]([S@@](=O)c2ccc(Nc3ncc4cc(Cl)c(=O)n(C5CCCC5)c4n3)c(C)c2)CC1]

In some embodiments, CBM is

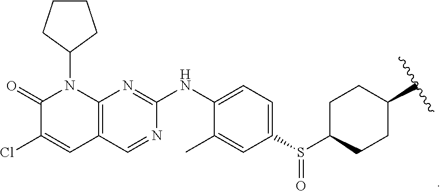 [SMILES: *[C@H]1CC[C@@H]([S@](=O)c2ccc(Nc3ncc4cc(Cl)c(=O)n(C5CCCC5)c4n3)c(C)c2)CC1]

In some embodiments, CBM is

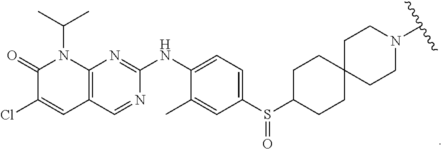 [SMILES: *N1CCC2(CCC(S(=O)c3ccc(Nc4ncc5cc(Cl)c(=O)n(C(C)C)c5n4)c(C)c3)CC2)CC1]

In some embodiments, CBM is

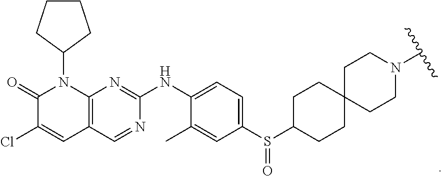 [SMILES: *N1CCC2(CCC(S(=O)c3ccc(Nc4ncc5cc(Cl)c(=O)n(C6CCCC6)c5n4)c(C)c3)CC2)CC1]

In some embodiments, CBM is

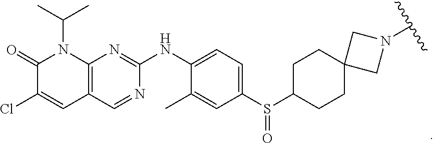 [SMILES: *N1CC2(CCC(S(=O)c3ccc(Nc4ncc5cc(Cl)c(=O)n(C(C)C)c5n4)c(C)c3)CC2)C1]

In some embodiments, CBM is

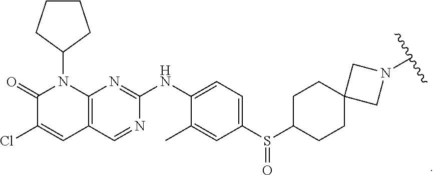 [SMILES: *N1CC2(CCC(S(=O)c3ccc(Nc4ncc5cc(Cl)c(=O)n(C6CCCC6)c5n4)c(C)c3)CC2)C1]

In some embodiments, CBM is

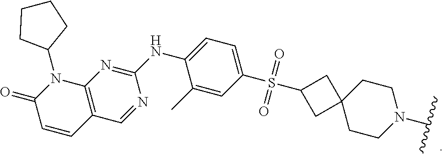 [SMILES: *N1CCC2(CC1)CC(S(=O)(=O)c1ccc(Nc3ncc4ccc(=O)n(C5CCCC5)c4n3)c(C)c1)C2]

In some embodiments, CBM is

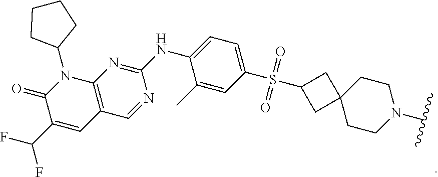 [SMILES: *N1CCC2(CC1)CC(S(=O)(=O)c1ccc(Nc3ncc4cc(C(F)F)c(=O)n(C5CCCC5)c4n3)c(C)c1)C2]

In some embodiments, CBM is

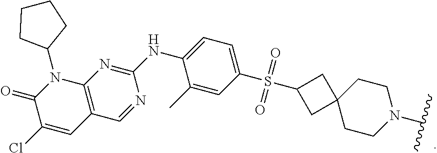 [SMILES: *N1CCC2(CC1)CC(S(=O)(=O)c1ccc(Nc3ncc4cc(Cl)c(=O)n(C5CCCC5)c4n3)c(C)c1)C2]

In some embodiments, CBM is

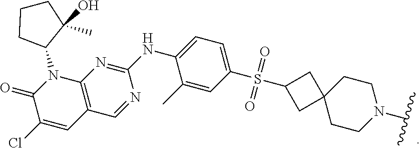 [SMILES: *N1CCC2(CC1)CC(S(=O)(=O)c1ccc(Nc3ncc4cc(Cl)c(=O)n([C@@H]5CCC[C@@]5(C)O)c4n3)c(C)c1)C2]

In some embodiments, CBM is

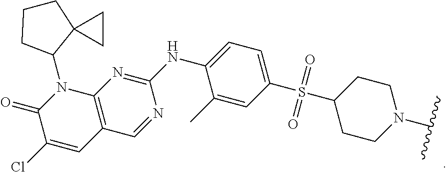 [SMILES: *N1CCC(S(=O)(=O)c2ccc(Nc3ncc4cc(Cl)c(=O)n(C5CCCC56CC6)c4n3)c(C)c2)CC1]

In some embodiments, CBM is

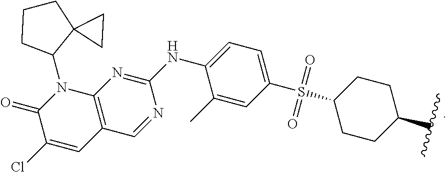 [SMILES: *[C@H]1CC[C@H](S(=O)(=O)c2ccc(Nc3ncc4cc(Cl)c(=O)n(C5CCCC56CC6)c4n3)c(C)c2)CC1]

In some embodiments, CBM is

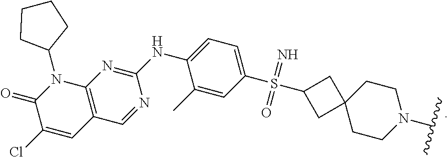 [SMILES: *N1CCC2(CC1)CC(S(=N)(=O)c1ccc(Nc3ncc4cc(Cl)c(=O)n(C5CCCC5)c4n3)c(C)c1)C2]

In some embodiments, CBM is

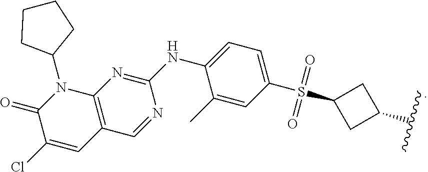 [SMILES: *[C@H]1C[C@H](S(=O)(=O)c2ccc(Nc3ncc4cc(Cl)c(=O)n(C5CCCC5)c4n3)c(C)c2)C1]

In some embodiments, CBM is

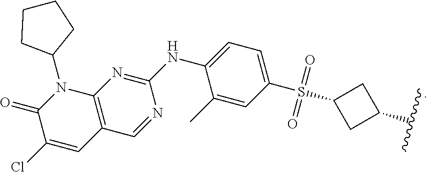 [SMILES: *[C@H]1C[C@@H](S(=O)(=O)c2ccc(Nc3ncc4cc(Cl)c(=O)n(C5CCCC5)c4n3)c(C)c2)C1]

In some embodiments, CBM is

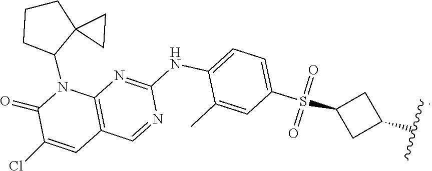 [SMILES: *[C@H]1C[C@H](S(=O)(=O)c2ccc(Nc3ncc4cc(Cl)c(=O)n(C5CCCC56CC6)c4n3)c(C)c2)C1]

In some embodiments, CBM is

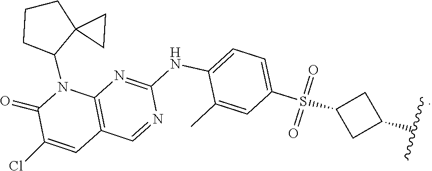 [SMILES: *[C@H]1C[C@@H](S(=O)(=O)c2ccc(Nc3ncc4cc(Cl)c(=O)n(C5CCCC56CC6)c4n3)c(C)c2)C1]

In some embodiments, CBM is

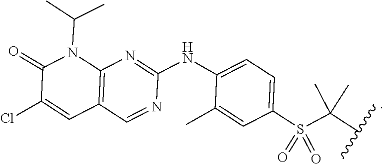 [SMILES: *C(C)(C)S(=O)(=O)c1ccc(Nc2ncc3cc(Cl)c(=O)n(C(C)C)c3n2)c(C)c1]

In some embodiments, CBM is

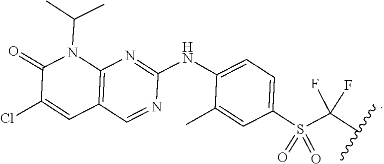 [SMILES: *C(F)(F)S(=O)(=O)c1ccc(Nc2ncc3cc(Cl)c(=O)n(C(C)C)c3n2)c(C)c1]

In some embodiments, CBM is

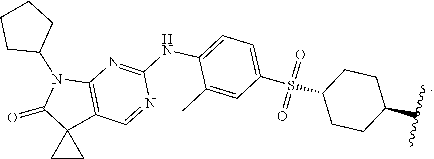 [SMILES: *[C@H]1CC[C@H](S(=O)(=O)c2ccc(Nc3ncc4c(n3)N(C3CCCC3)C(=O)C43CC3)c(C)c2)CC1]

In some embodiments, CBM is

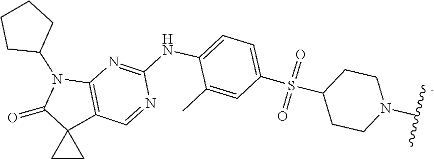 [SMILES: *N1CCC(S(=O)(=O)c2ccc(Nc3ncc4c(n3)N(C3CCCC3)C(=O)C43CC3)c(C)c2)CC1]

In some embodiments, CBM is

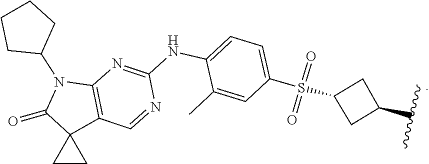 [SMILES: *[C@H]1C[C@H](S(=O)(=O)c2ccc(Nc3ncc4c(n3)N(C3CCCC3)C(=O)C43CC3)c(C)c2)C1]

In some embodiments, CBM is

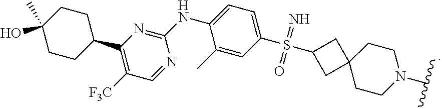 [SMILES: *N1CCC2(CC1)CC(S(=N)(=O)c1ccc(Nc3ncc(C(F)(F)F)c([C@H]4CC[C@@](C)(O)CC4)n3)c(C)c1)C2]

In some embodiments, CBM is

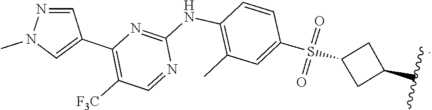 [SMILES: *[C@H]1C[C@H](S(=O)(=O)c2ccc(Nc3ncc(C(F)(F)F)c(-c4cnn(C)c4)n3)c(C)c2)C1]

In some embodiments, CBM is

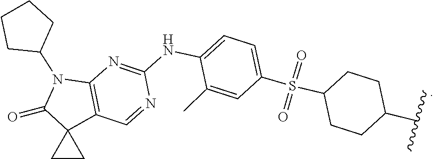 [SMILES: *C1CCC(S(=O)(=O)c2ccc(Nc3ncc4c(n3)N(C3CCCC3)C(=O)C43CC3)c(C)c2)CC1]

In some embodiments, CBM is

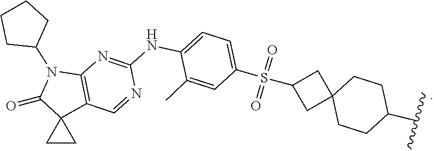 [SMILES: *C1CCC2(CC1)CC(S(=O)(=O)c1ccc(Nc3ncc4c(n3)N(C3CCCC3)C(=O)C43CC3)c(C)c1)C2]

In some embodiments, CBM is

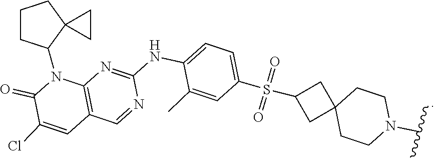 [SMILES: *N1CCC2(CC1)CC(S(=O)(=O)c1ccc(Nc3ncc4cc(Cl)c(=O)n(C5CCCC56CC6)c4n3)c(C)c1)C2]

In some embodiments, CBM is

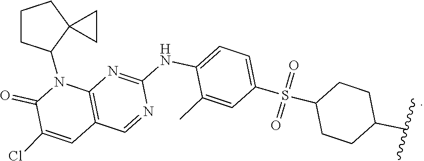 [SMILES: *C1CCC(S(=O)(=O)c2ccc(Nc3ncc4cc(Cl)c(=O)n(C5CCCC56CC6)c4n3)c(C)c2)CC1]

In some embodiments, CBM is

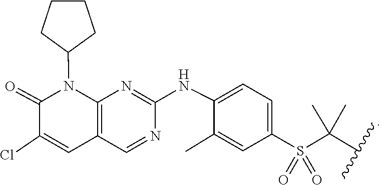 [SMILES: *C(C)(C)S(=O)(=O)c1ccc(Nc2ncc3cc(Cl)c(=O)n(C4CCCC4)c3n2)c(C)c1]

In some embodiments, CBM is

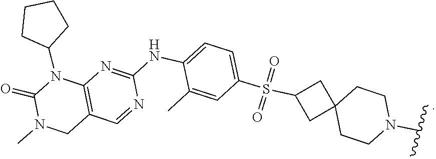 [SMILES: *N1CCC2(CC1)CC(S(=O)(=O)c1ccc(Nc3ncc4c(n3)N(C3CCCC3)C(=O)N(C)C4)c(C)c1)C2]

In some embodiments, CBM is

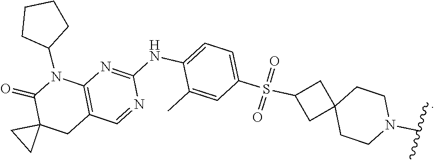 [SMILES: *N1CCC2(CC1)CC(S(=O)(=O)c1ccc(Nc3ncc4c(n3)N(C3CCCC3)C(=O)C3(CC3)C4)c(C)c1)C2]

In some embodiments, CBM is

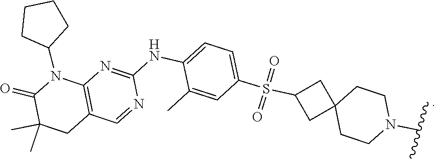 [SMILES: *N1CCC2(CC1)CC(S(=O)(=O)c1ccc(Nc3ncc4c(n3)N(C3CCCC3)C(=O)C(C)(C)C4)c(C)c1)C2]

In some embodiments, CBM is

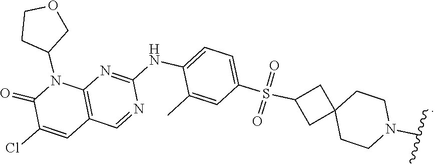 [SMILES: *N1CCC2(CC1)CC(S(=O)(=O)c1ccc(Nc3ncc4cc(Cl)c(=O)n(C5CCOC5)c4n3)c(C)c1)C2]

In some embodiments, CBM is

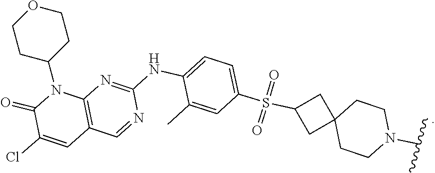 [SMILES: *N1CCC2(CC1)CC(S(=O)(=O)c1ccc(Nc3ncc4cc(Cl)c(=O)n(C5CCOCC5)c4n3)c(C)c1)C2]

In some embodiments, CBM is

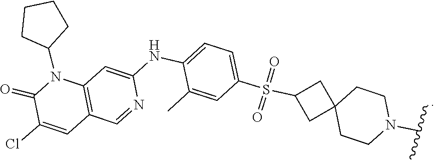 [SMILES: *N1CCC2(CC1)CC(S(=O)(=O)c1ccc(Nc3cc4c(cn3)cc(Cl)c(=O)n4C3CCCC3)c(C)c1)C2]

In some embodiments, CBM is

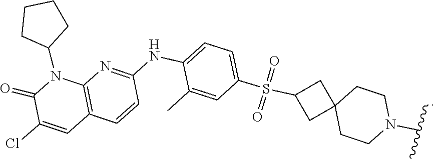 [SMILES: *N1CCC2(CC1)CC(S(=O)(=O)c1ccc(Nc3ccc4cc(Cl)c(=O)n(C5CCCC5)c4n3)c(C)c1)C2]

In some embodiments, CBM is

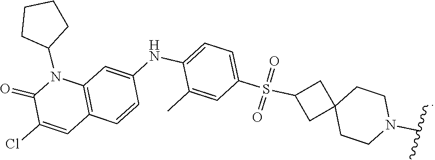 [SMILES: *N1CCC2(CC1)CC(S(=O)(=O)c1ccc(Nc3ccc4cc(Cl)c(=O)n(C5CCCC5)c4c3)c(C)c1)C2]

In some embodiments, CBM is

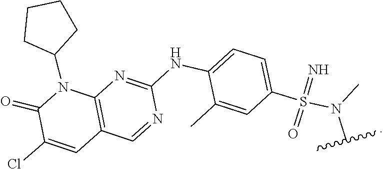 [SMILES: *N(C)S(=N)(=O)c1ccc(Nc2ncc3cc(Cl)c(=O)n(C4CCCC4)c3n2)c(C)c1]

In some embodiments, CBM is

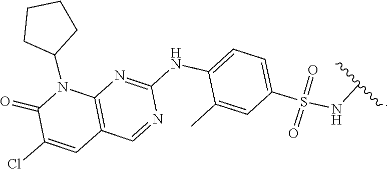 [SMILES: *NS(=O)(=O)c1ccc(Nc2ncc3cc(Cl)c(=O)n(C4CCCC4)c3n2)c(C)c1]

In some embodiments, CBM is

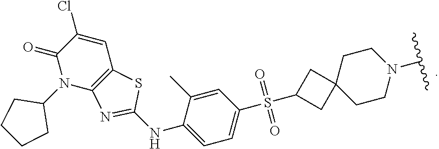 [SMILES: *N1CCC2(CC1)CC(S(=O)(=O)c1ccc(Nc3nc4c(cc(Cl)c(=O)n4C4CCCC4)s3)c(C)c1)C2]

In some embodiments, CBM is

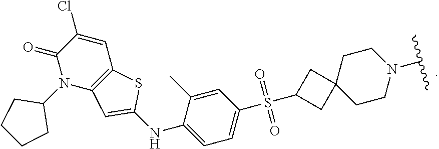 [SMILES: *N1CCC2(CC1)CC(S(=O)(=O)c1ccc(Nc3cc4c(cc(Cl)c(=O)n4C4CCCC4)s3)c(C)c1)C2]

In some embodiments, CBM is

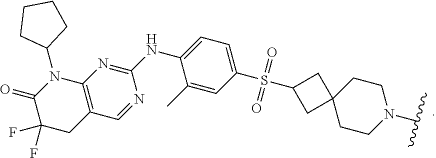 [SMILES: *N1CCC2(CC1)CC(S(=O)(=O)c1ccc(Nc3ncc4c(n3)N(C3CCCC3)C(=O)C(F)(F)C4)c(C)c1)C2]

In some embodiments, CBM is

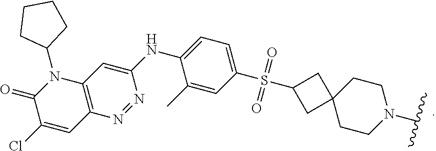 [SMILES: *N1CCC2(CC1)CC(S(=O)(=O)c1ccc(Nc3cc4c(cc(Cl)c(=O)n4C4CCCC4)nn3)c(C)c1)C2]

In some embodiments, CBM is

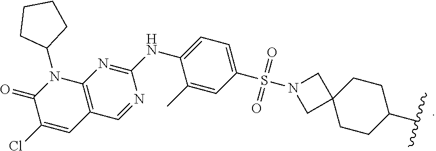 [SMILES: *C1CCC2(CC1)CN(S(=O)(=O)c1ccc(Nc3ncc4cc(Cl)c(=O)n(C5CCCC5)c4n3)c(C)c1)C2]

In some embodiments, CBM is

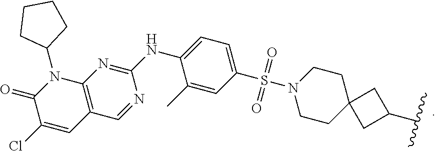 [SMILES: *C1CC2(CCN(S(=O)(=O)c3ccc(Nc4ncc5cc(Cl)c(=O)n(C6CCCC6)c5n4)c(C)c3)CC2)C1]

In some embodiments, CBM is

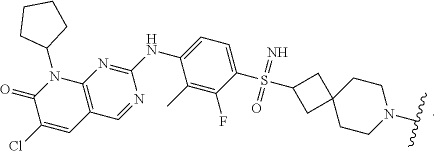 [SMILES: *N1CCC2(CC1)CC(S(=N)(=O)c1ccc(Nc3ncc4cc(Cl)c(=O)n(C5CCCC5)c4n3)c(C)c1F)C2]

In some embodiments, CBM is

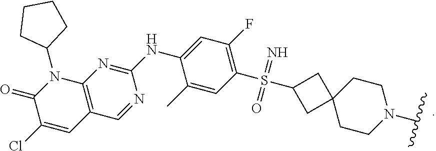 [SMILES: *N1CCC2(CC1)CC(S(=N)(=O)c1cc(C)c(Nc3ncc4cc(Cl)c(=O)n(C5CCCC5)c4n3)cc1F)C2]

In some embodiments, CBM is

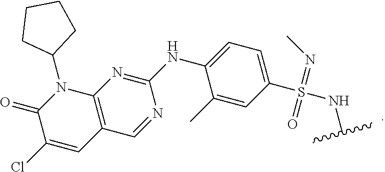 [SMILES: *NS(=O)(=NC)c1ccc(Nc2ncc3cc(Cl)c(=O)n(C4CCCC4)c3n2)c(C)c1]

In some embodiments, CBM is

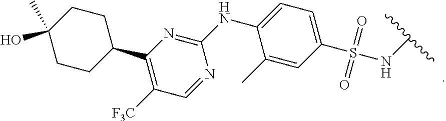 [SMILES: *NS(=O)(=O)c1ccc(Nc2ncc(C(F)(F)F)c([C@H]3CC[C@@](C)(O)CC3)n2)c(C)c1]

In some embodiments, CBM is

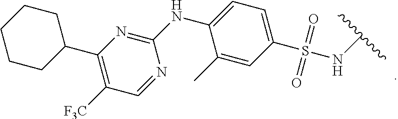 [SMILES: *NS(=O)(=O)c1ccc(Nc2ncc(C(F)(F)F)c(C3CCCCC3)n2)c(C)c1]

In some embodiments, CBM is

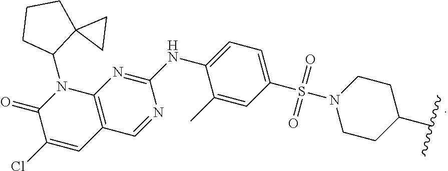 [SMILES: *C1CCN(S(=O)(=O)c2ccc(Nc3ncc4cc(Cl)c(=O)n(C5CCCC56CC6)c4n3)c(C)c2)CC1]

In some embodiments, CBM is

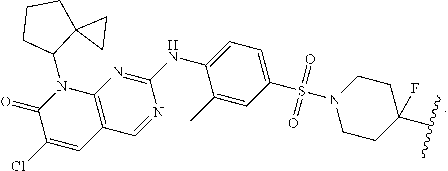 [SMILES: *C1(F)CCN(S(=O)(=O)c2ccc(Nc3ncc4cc(Cl)c(=O)n(C5CCCC56CC6)c4n3)c(C)c2)CC1]

In some embodiments, CBM is

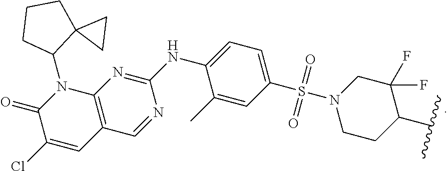 [SMILES: *C1CCN(S(=O)(=O)c2ccc(Nc3ncc4cc(Cl)c(=O)n(C5CCCC56CC6)c4n3)c(C)c2)CC1(F)F]

In some embodiments, CBM is

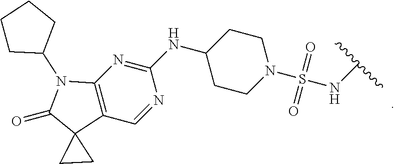 [SMILES: *NS(=O)(=O)N1CCC(Nc2ncc3c(n2)N(C2CCCC2)C(=O)C32CC2)CC1]

In some embodiments, CBM is

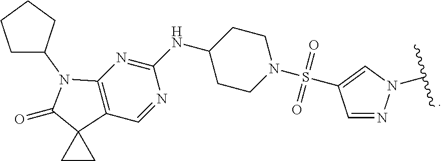 [SMILES: *n1cc(S(=O)(=O)N2CCC(Nc3ncc4c(n3)N(C3CCCC3)C(=O)C43CC3)CC2)cn1]

In some embodiments, CBM is

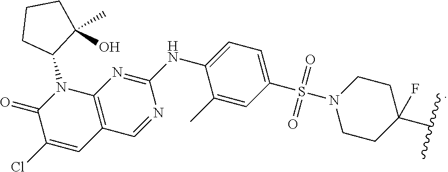 [SMILES: *C1(F)CCN(S(=O)(=O)c2ccc(Nc3ncc4cc(Cl)c(=O)n([C@@H]5CCC[C@@]5(C)O)c4n3)c(C)c2)CC1]

In some embodiments, CBM is

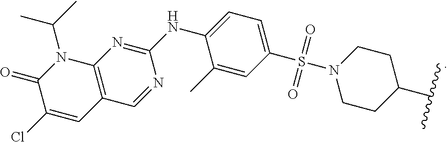 [SMILES: *C1CCN(S(=O)(=O)c2ccc(Nc3ncc4cc(Cl)c(=O)n(C(C)C)c4n3)c(C)c2)CC1]

In some embodiments, CBM is

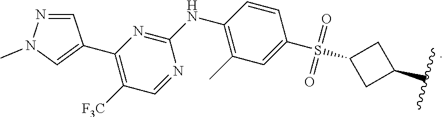 [SMILES: *[C@H]1C[C@H](S(=O)(=O)c2ccc(Nc3ncc(C(F)(F)F)c(-c4cnn(C)c4)n3)c(C)c2)C1]

In some embodiments, CBM is

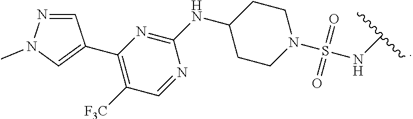 [SMILES: *NS(=O)(=O)N1CCC(Nc2ncc(C(F)(F)F)c(-c3cnn(C)c3)n2)CC1]

In some embodiments, CBM is

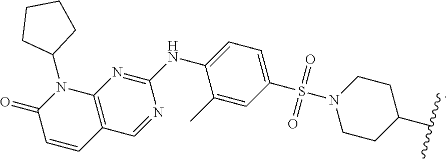 [SMILES: *C1CCN(S(=O)(=O)c2ccc(Nc3ncc4ccc(=O)n(C5CCCC5)c4n3)c(C)c2)CC1]

In some embodiments, CBM is

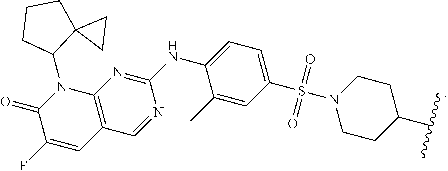 [SMILES: *C1CCN(S(=O)(=O)c2ccc(Nc3ncc4cc(F)c(=O)n(C5CCCC56CC6)c4n3)c(C)c2)CC1]

In some embodiments, CBM is

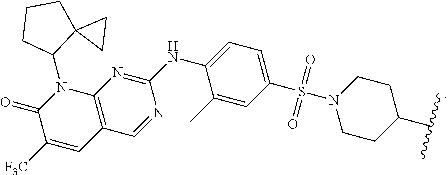 [SMILES: *C1CCN(S(=O)(=O)c2ccc(Nc3ncc4cc(C(F)(F)F)c(=O)n(C5CCCC56CC6)c4n3)c(C)c2)CC1]

In some embodiments, CBM is

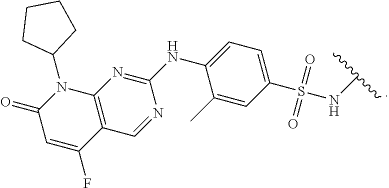 [SMILES: *NS(=O)(=O)c1ccc(Nc2ncc3c(F)cc(=O)n(C4CCCC4)c3n2)c(C)c1]

In some embodiments, CBM is

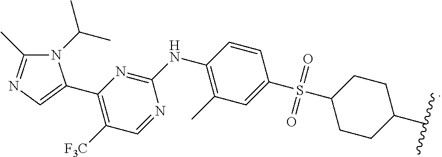 [SMILES: *C1CCC(S(=O)(=O)c2ccc(Nc3ncc(C(F)(F)F)c(-c4cnc(C)n4C(C)C)n3)c(C)c2)CC1]

In some embodiments, CBM is

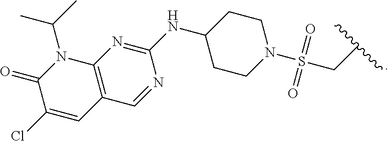 [SMILES: *CS(=O)(=O)N1CCC(Nc2ncc3cc(Cl)c(=O)n(C(C)C)c3n2)CC1]

In some embodiments, CBM is

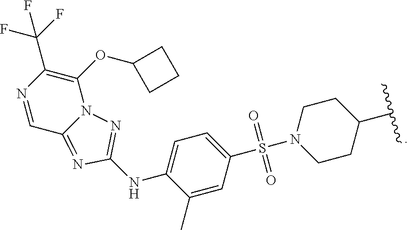 [SMILES: *C1CCN(S(=O)(=O)c2ccc(Nc3nc4cnc(C(F)(F)F)c(OC5CCC5)n4n3)c(C)c2)CC1]

In some embodiments, CBM is

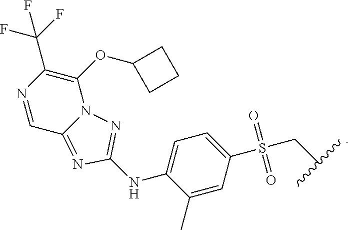 [SMILES: *CS(=O)(=O)c1ccc(Nc2nc3cnc(C(F)(F)F)c(OC4CCC4)n3n2)c(C)c1]

In some embodiments, CBM is

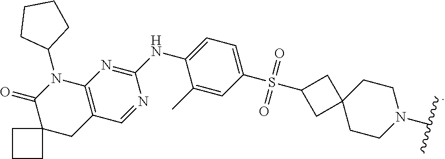 [SMILES: *N1CCC2(CC1)CC(S(=O)(=O)c1ccc(Nc3ncc4c(n3)N(C3CCCC3)C(=O)C3(CCC3)C4)c(C)c1)C2]

In some embodiments, CBM is

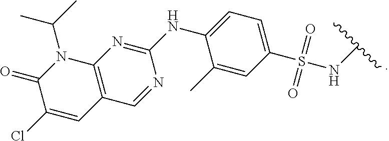 [SMILES: *NS(=O)(=O)c1ccc(Nc2ncc3cc(Cl)c(=O)n(C(C)C)c3n2)c(C)c1]

In some embodiments, CBM is

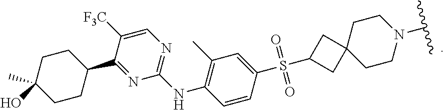 [SMILES: *N1CCC2(CC1)CC(S(=O)(=O)c1ccc(Nc3ncc(C(F)(F)F)c([C@H]4CC[C@@](C)(O)CC4)n3)c(C)c1)C2]

In some embodiments, CBM is

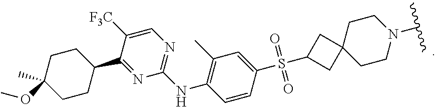 [SMILES: *N1CCC2(CC1)CC(S(=O)(=O)c1ccc(Nc3ncc(C(F)(F)F)c([C@H]4CC[C@@](C)(OC)CC4)n3)c(C)c1)C2]

In some embodiments, CBM is

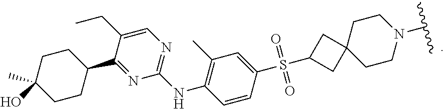 [SMILES: *N1CCC2(CC1)CC(S(=O)(=O)c1ccc(Nc3ncc(CC)c([C@H]4CC[C@@](C)(O)CC4)n3)c(C)c1)C2]

In some embodiments, CBM is

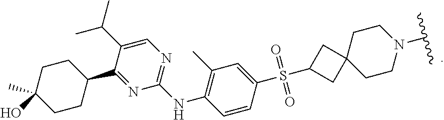 [SMILES: *N1CCC2(CC1)CC(S(=O)(=O)c1ccc(Nc3ncc(C(C)C)c([C@H]4CC[C@@](C)(O)CC4)n3)c(C)c1)C2]

In some embodiments, CBM is

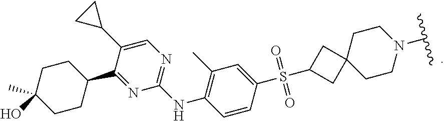 [SMILES: *N1CCC2(CC1)CC(S(=O)(=O)c1ccc(Nc3ncc(C4CC4)c([C@H]4CC[C@@](C)(O)CC4)n3)c(C)c1)C2]

In some embodiments, CBM is

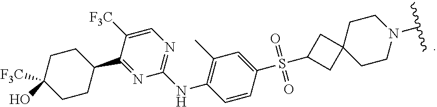 [SMILES: *N1CCC2(CC1)CC(S(=O)(=O)c1ccc(Nc3ncc(C(F)(F)F)c([C@H]4CC[C@](O)(C(F)(F)F)CC4)n3)c(C)c1)C2]

In some embodiments, CBM is

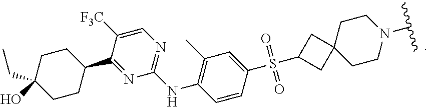 [SMILES: *N1CCC2(CC1)CC(S(=O)(=O)c1ccc(Nc3ncc(C(F)(F)F)c([C@H]4CC[C@](O)(CC)CC4)n3)c(C)c1)C2]

In some embodiments, CBM is

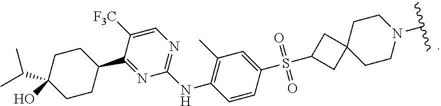 [SMILES: *N1CCC2(CC1)CC(S(=O)(=O)c1ccc(Nc3ncc(C(F)(F)F)c([C@H]4CC[C@](O)(C(C)C)CC4)n3)c(C)c1)C2]

In some embodiments, CBM is

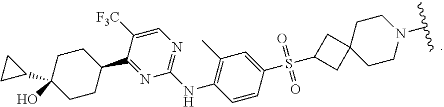 [SMILES: *N1CCC2(CC1)CC(S(=O)(=O)c1ccc(Nc3ncc(C(F)(F)F)c([C@H]4CC[C@](O)(C5CC5)CC4)n3)c(C)c1)C2]

In some embodiments, CBM is

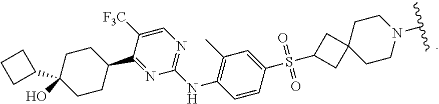 [SMILES: *N1CCC2(CC1)CC(S(=O)(=O)c1ccc(Nc3ncc(C(F)(F)F)c([C@H]4CC[C@](O)(C5CCC5)CC4)n3)c(C)c1)C2]

In some embodiments, CBM is

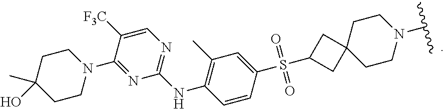 [SMILES: *N1CCC2(CC1)CC(S(=O)(=O)c1ccc(Nc3ncc(C(F)(F)F)c(N4CCC(C)(O)CC4)n3)c(C)c1)C2]

In some embodiments, CBM is

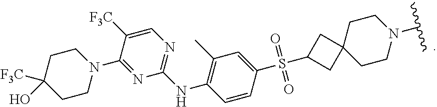 [SMILES: *N1CCC2(CC1)CC(S(=O)(=O)c1ccc(Nc3ncc(C(F)(F)F)c(N4CCC(O)(C(F)(F)F)CC4)n3)c(C)c1)C2]

In some embodiments, CBM is

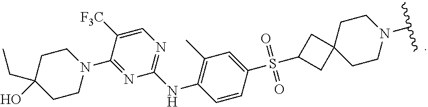 [SMILES: *N1CCC2(CC1)CC(S(=O)(=O)c1ccc(Nc3ncc(C(F)(F)F)c(N4CCC(O)(CC)CC4)n3)c(C)c1)C2]

In some embodiments, CBM is

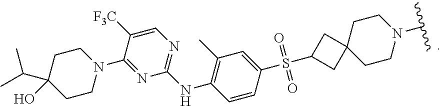 [SMILES: *N1CCC2(CC1)CC(S(=O)(=O)c1ccc(Nc3ncc(C(F)(F)F)c(N4CCC(O)(C(C)C)CC4)n3)c(C)c1)C2]

In some embodiments, CBM is

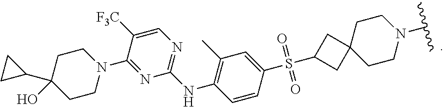 [SMILES: *N1CCC2(CC1)CC(S(=O)(=O)c1ccc(Nc3ncc(C(F)(F)F)c(N4CCC(O)(C5CC5)CC4)n3)c(C)c1)C2]

In some embodiments, CBM is

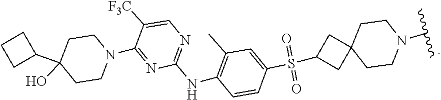 [SMILES: *N1CCC2(CC1)CC(S(=O)(=O)c1ccc(Nc3ncc(C(F)(F)F)c(N4CCC(O)(C5CCC5)CC4)n3)c(C)c1)C2]

In some embodiments, CBM is

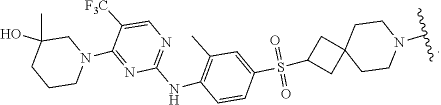 [SMILES: *N1CCC2(CC1)CC(S(=O)(=O)c1ccc(Nc3ncc(C(F)(F)F)c(N4CCCC(C)(O)C4)n3)c(C)c1)C2]

In some embodiments, CBM is

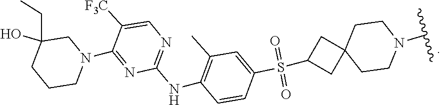 [SMILES: *N1CCC2(CC1)CC(S(=O)(=O)c1ccc(Nc3ncc(C(F)(F)F)c(N4CCCC(O)(CC)C4)n3)c(C)c1)C2]

In some embodiments, CBM is

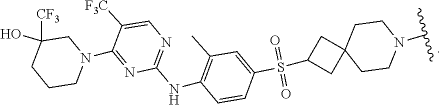 [SMILES: *N1CCC2(CC1)CC(S(=O)(=O)c1ccc(Nc3ncc(C(F)(F)F)c(N4CCCC(O)(C(F)(F)F)C4)n3)c(C)c1)C2]

In some embodiments, CBM is

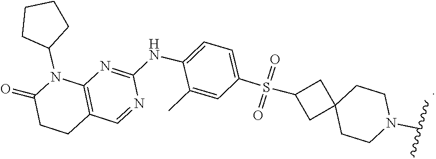 [SMILES: *N1CCC2(CC1)CC(S(=O)(=O)c1ccc(Nc3ncc4c(n3)N(C3CCCC3)C(=O)CC4)c(C)c1)C2]

In some embodiments, CBM is

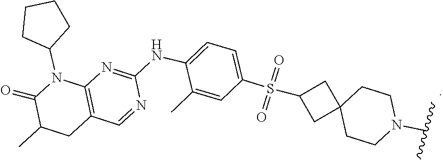 [SMILES: *N1CCC2(CC1)CC(S(=O)(=O)c1ccc(Nc3ncc4c(n3)N(C3CCCC3)C(=O)C(C)C4)c(C)c1)C2]

In some embodiments, CBM is

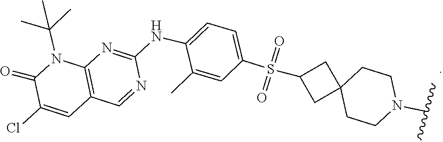 [SMILES: *N1CCC2(CC1)CC(S(=O)(=O)c1ccc(Nc3ncc4cc(Cl)c(=O)n(C(C)(C)C)c4n3)c(C)c1)C2]

In some embodiments, CBM is

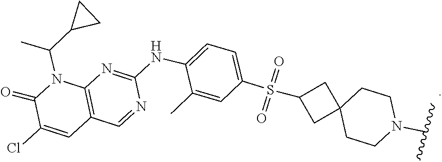 [SMILES: *N1CCC2(CC1)CC(S(=O)(=O)c1ccc(Nc3ncc4cc(Cl)c(=O)n(C(C)C5CC5)c4n3)c(C)c1)C2]

In some embodiments, CBM is

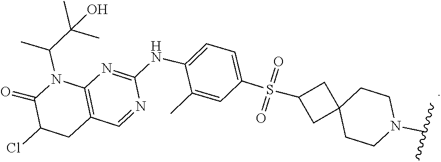 [SMILES: *N1CCC2(CC1)CC(S(=O)(=O)c1ccc(Nc3ncc4c(n3)N(C(C)C(C)(C)O)C(=O)C(Cl)C4)c(C)c1)C2]

In some embodiments, CBM is

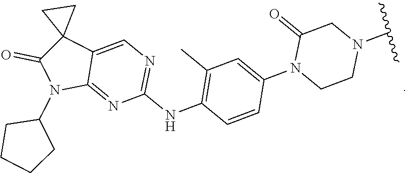 [SMILES: *N1CCN(c2ccc(Nc3ncc4c(n3)N(C3CCCC3)C(=O)C43CC3)c(C)c2)C(=O)C1]

In some embodiments, CBM is

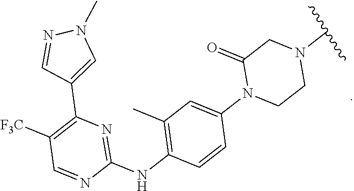 [SMILES: *N1CCN(c2ccc(Nc3ncc(C(F)(F)F)c(-c4cnn(C)c4)n3)c(C)c2)C(=O)C1]

In some embodiments CBM is

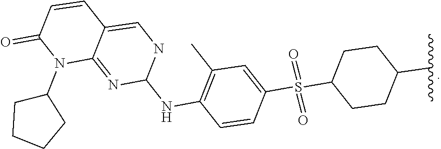

In some embodiments, CBM is

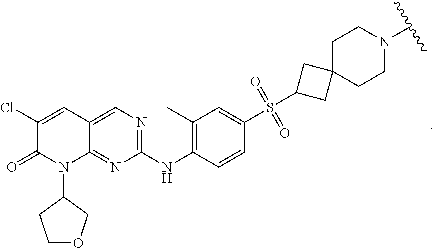 [SMILES: *N1CCC2(CC1)CC(S(=O)(=O)c1ccc(Nc3ncc4cc(Cl)c(=O)n(C5CCOC5)c4n3)c(C)c1)C2]

In some embodiments, CBM is

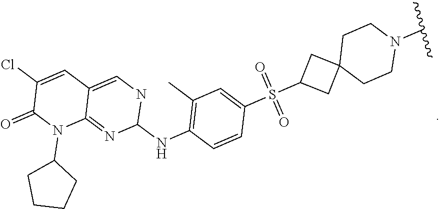

In some embodiments, CBM is

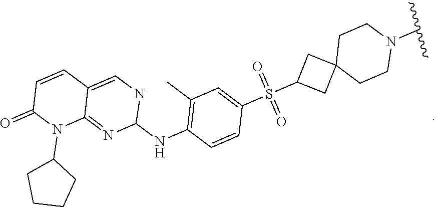

In some embodiments, CBM is

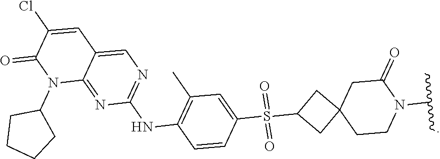 [SMILES: *N1CCC2(CC1=O)CC(S(=O)(=O)c1ccc(Nc3ncc4cc(Cl)c(=O)n(C5CCCC5)c4n3)c(C)c1)C2]

In some embodiments, CBM is

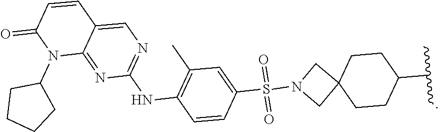 [SMILES: *C1CCC2(CC1)CN(S(=O)(=O)c1ccc(Nc3ncc4ccc(=O)n(C5CCCC5)c4n3)c(C)c1)C2]

In some embodiments, CBM is

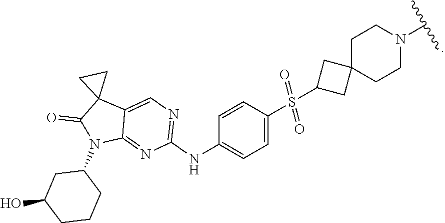 [SMILES: *N1CCC2(CC1)CC(S(=O)(=O)c1ccc(Nc3ncc4c(n3)N([C@@H]3CCC[C@@H](O)C3)C(=O)C43CC3)cc1)C2]

In some embodiments, CBM is

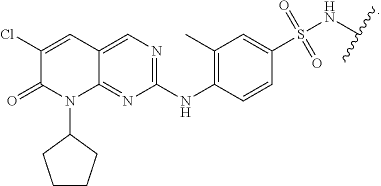 [SMILES: *NS(=O)(=O)c1ccc(Nc2ncc3cc(Cl)c(=O)n(C4CCCC4)c3n2)c(C)c1]

In some embodiments, CBM is

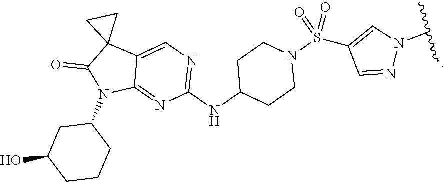 [SMILES: *n1cc(S(=O)(=O)N2CCC(Nc3ncc4c(n3)N([C@@H]3CCC[C@@H](O)C3)C(=O)C43CC3)CC2)cn1]

In some embodiments, CBM is

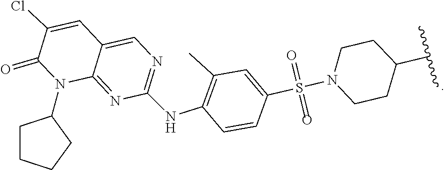

In some embodiments, CBM is

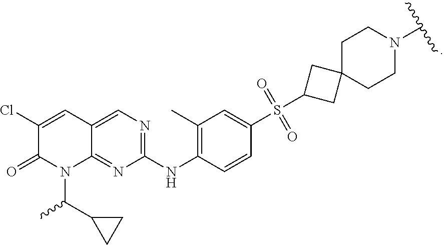 [SMILES: *N1CCC2(CC1)CC(S(=O)(=O)c1ccc(Nc3ncc4cc(Cl)c(=O)n(C(C)C5CC5)c4n3)c(C)c1)C2]

In some embodiments, CBM is

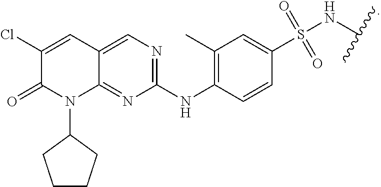 [SMILES: *NS(=O)(=O)c1ccc(Nc2ncc3cc(Cl)c(=O)n(C4CCCC4)c3n2)c(C)c1]

In some embodiments, CBM is

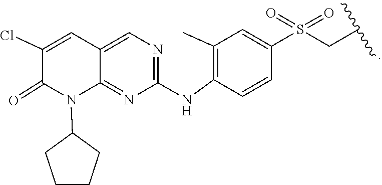 [SMILES: *CS(=O)(=O)c1ccc(Nc2ncc3cc(Cl)c(=O)n(C4CCCC4)c3n2)c(C)c1]

In some embodiments, CBM is

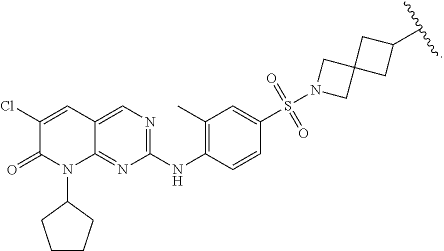 [SMILES: *C1CC2(C1)CN(S(=O)(=O)c1ccc(Nc3ncc4cc(Cl)c(=O)n(C5CCCC5)c4n3)c(C)c1)C2]

In some embodiments, CBM is

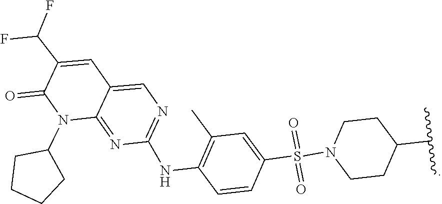 [SMILES: *C1CCN(S(=O)(=O)c2ccc(Nc3ncc4cc(C(F)F)c(=O)n(C5CCCC5)c4n3)c(C)c2)CC1]

In some embodiments, CBM is

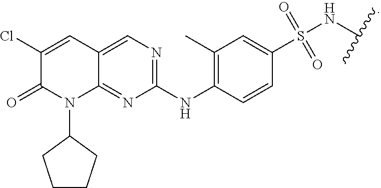 [SMILES: *NS(=O)(=O)c1ccc(Nc2ncc3cc(Cl)c(=O)n(C4CCCC4)c3n2)c(C)c1]

In some embodiments, CBM is

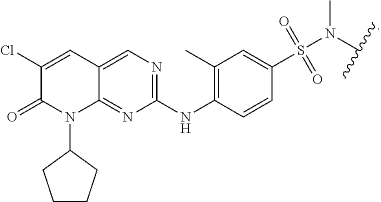 [SMILES: *N(C)S(=O)(=O)c1ccc(Nc2ncc3cc(Cl)c(=O)n(C4CCCC4)c3n2)c(C)c1]

In some embodiments, CBM is

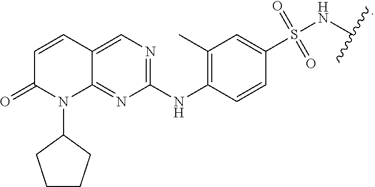 [SMILES: *NS(=O)(=O)c1ccc(Nc2ncc3ccc(=O)n(C4CCCC4)c3n2)c(C)c1]

In some embodiments, CBM is

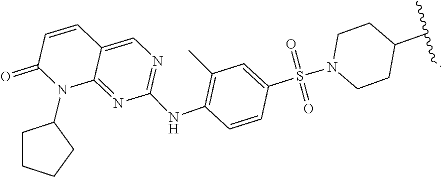 [SMILES: *C1CCN(S(=O)(=O)c2ccc(Nc3ncc4ccc(=O)n(C5CCCC5)c4n3)c(C)c2)CC1]

In some embodiments, CBM is

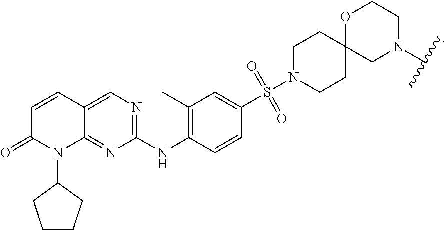 [SMILES: *N1CCOC2(CCN(S(=O)(=O)c3ccc(Nc4ncc5ccc(=O)n(C6CCCC6)c5n4)c(C)c3)CC2)C1]

In some embodiments, CBM is

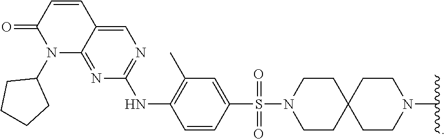 [SMILES: *N1CCC2(CC1)CCN(S(=O)(=O)c1ccc(Nc3ncc4ccc(=O)n(C5CCCC5)c4n3)c(C)c1)CC2]

In some embodiments, CBM is

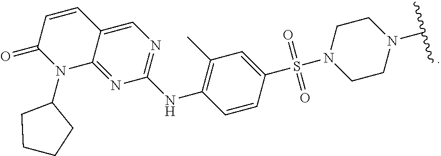 [SMILES: *N1CCN(S(=O)(=O)c2ccc(Nc3ncc4ccc(=O)n(C5CCCC5)c4n3)c(C)c2)CC1]

In some embodiments, CBM is

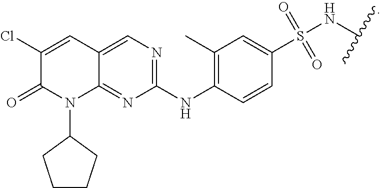 [SMILES: *NS(=O)(=O)c1ccc(Nc2ncc3cc(Cl)c(=O)n(C4CCCC4)c3n2)c(C)c1]

In some embodiments, CBM is

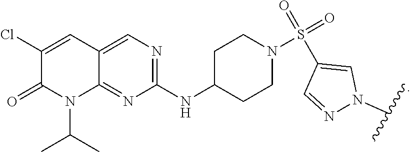 [SMILES: *n1cc(S(=O)(=O)N2CCC(Nc3ncc4cc(Cl)c(=O)n(C(C)C)c4n3)CC2)cn1]

In some embodiments, CBM is

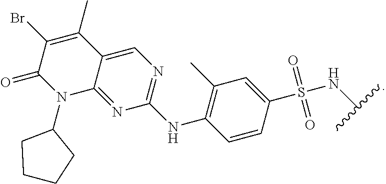 [SMILES: *NS(=O)(=O)c1ccc(Nc2ncc3c(C)c(Br)c(=O)n(C4CCCC4)c3n2)c(C)c1]

In some embodiments, CBM is

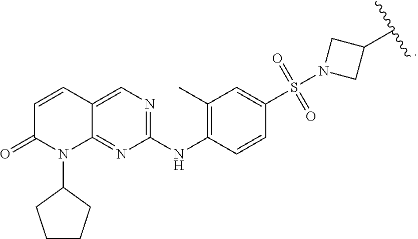 [SMILES: *C1CN(S(=O)(=O)c2ccc(Nc3ncc4ccc(=O)n(C5CCCC5)c4n3)c(C)c2)C1]

In some embodiments, CBM is

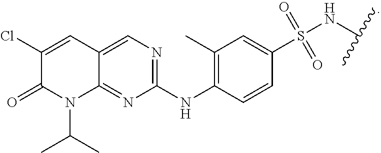 [SMILES: *NS(=O)(=O)c1ccc(Nc2ncc3cc(Cl)c(=O)n(C(C)C)c3n2)c(C)c1]

In some embodiments, CBM is

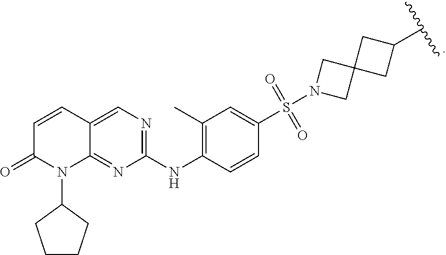 [SMILES: *C1CC2(C1)CN(S(=O)(=O)c1ccc(Nc3ncc4ccc(=O)n(C5CCCC5)c4n3)c(C)c1)C2]

In some embodiments, CBM is

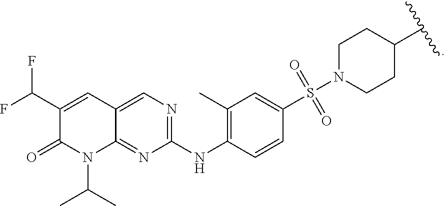 [SMILES: *C1CCN(S(=O)(=O)c2ccc(Nc3ncc4cc(C(F)F)c(=O)n(C(C)C)c4n3)c(C)c2)CC1]

In some embodiments, CBM is

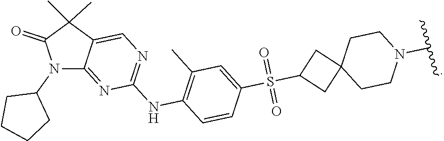 [SMILES: *N1CCC2(CC1)CC(S(=O)(=O)c1ccc(Nc3ncc4c(n3)N(C3CCCC3)C(=O)C4(C)C)c(C)c1)C2]

In some embodiments, CBM is

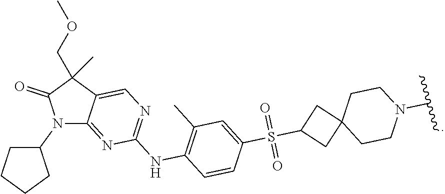 [SMILES: *N1CCC2(CC1)CC(S(=O)(=O)c1ccc(Nc3ncc4c(n3)N(C3CCCC3)C(=O)C4(C)COC)c(C)c1)C2]

In some embodiments, CBM is

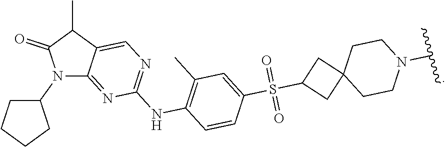 [SMILES: *N1CCC2(CC1)CC(S(=O)(=O)c1ccc(Nc3ncc4c(n3)N(C3CCCC3)C(=O)C4C)c(C)c1)C2]

In some embodiments, CBM is

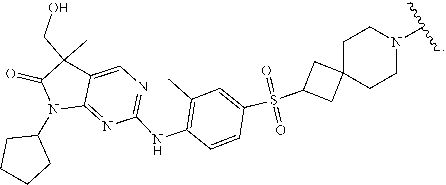 [SMILES: *N1CCC2(CC1)CC(S(=O)(=O)c1ccc(Nc3ncc4c(n3)N(C3CCCC3)C(=O)C4(C)CO)c(C)c1)C2]

In some embodiments, CBM is

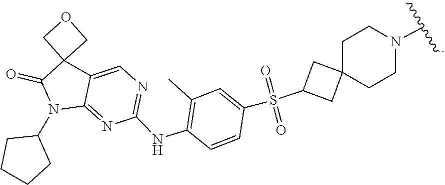 [SMILES: *N1CCC2(CC1)CC(S(=O)(=O)c1ccc(Nc3ncc4c(n3)N(C3CCCC3)C(=O)C43COC3)c(C)c1)C2]

In some embodiments, CBM is

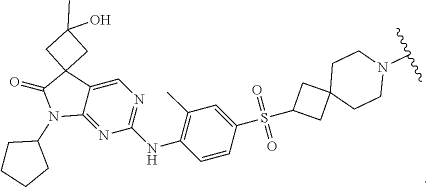 [SMILES: *N1CCC2(CC1)CC(S(=O)(=O)c1ccc(Nc3ncc4c(n3)N(C3CCCC3)C(=O)C43CC(C)(O)C3)c(C)c1)C2]

In some embodiments, CBM is

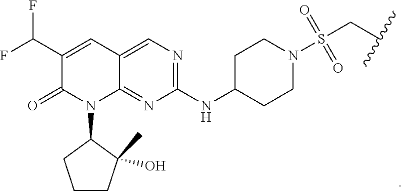 [SMILES: *CS(=O)(=O)N1CCC(Nc2ncc3cc(C(F)F)c(=O)n([C@@H]4CCC[C@@]4(C)O)c3n2)CC1]

In some embodiments, CBM is

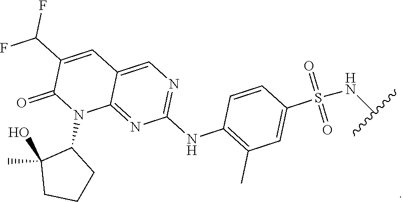 [SMILES: *NS(=O)(=O)c1ccc(Nc2ncc3cc(C(F)F)c(=O)n([C@@H]4CCC[C@@]4(C)O)c3n2)c(C)c1]

In some embodiments, CBM is

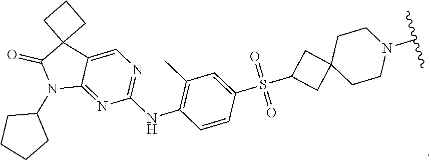 [SMILES: *N1CCC2(CC1)CC(S(=O)(=O)c1ccc(Nc3ncc4c(n3)N(C3CCCC3)C(=O)C43CCC3)c(C)c1)C2]

In some embodiments, CBM is

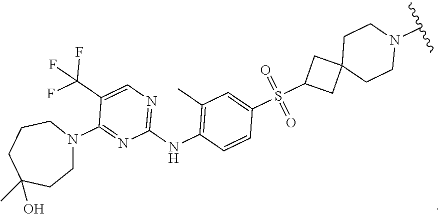 [SMILES: *N1CCC2(CC1)CC(S(=O)(=O)c1ccc(Nc3ncc(C(F)(F)F)c(N4CCCC(C)(O)CC4)n3)c(C)c1)C2]

In some embodiments, CBM is

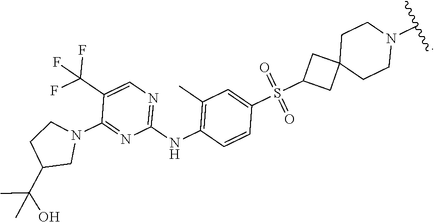 [SMILES: *N1CCC2(CC1)CC(S(=O)(=O)c1ccc(Nc3ncc(C(F)(F)F)c(N4CCC(C(C)(C)O)C4)n3)c(C)c1)C2]

In some embodiments, CBM is

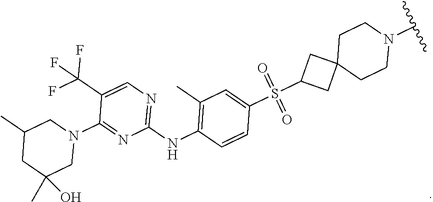 [SMILES: *N1CCC2(CC1)CC(S(=O)(=O)c1ccc(Nc3ncc(C(F)(F)F)c(N4CC(C)CC(C)(O)C4)n3)c(C)c1)C2]

In some embodiments, CBM is

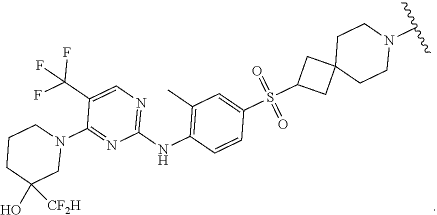 [SMILES: *N1CCC2(CC1)CC(S(=O)(=O)c1ccc(Nc3ncc(C(F)(F)F)c(N4CCCC(O)(C(F)F)C4)n3)c(C)c1)C2]

In some embodiments, CBM is

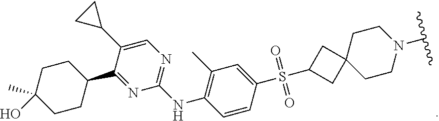 [SMILES: *N1CCC2(CC1)CC(S(=O)(=O)c1ccc(Nc3ncc(C4CC4)c([C@H]4CC[C@@](C)(O)CC4)n3)c(C)c1)C2]

In some embodiments, CBM is

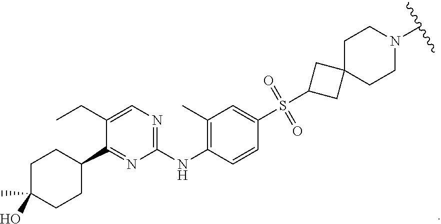 [SMILES: *N1CCC2(CC1)CC(S(=O)(=O)c1ccc(Nc3ncc(CC)c([C@H]4CC[C@@](C)(O)CC4)n3)c(C)c1)C2]

In some embodiments, CBM is

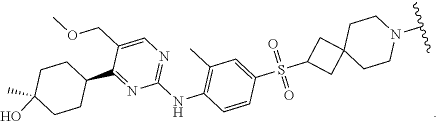 [SMILES: *N1CCC2(CC1)CC(S(=O)(=O)c1ccc(Nc3ncc(COC)c([C@H]4CC[C@@](C)(O)CC4)n3)c(C)c1)C2]

In some embodiments, CBM is

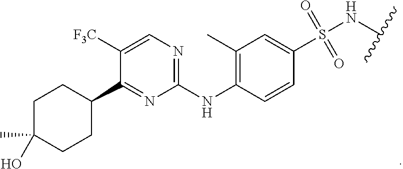 [SMILES: *NS(=O)(=O)c1ccc(Nc2ncc(C(F)(F)F)c([C@H]3CC[C@@](C)(O)CC3)n2)c(C)c1]

In some embodiments, CBM is

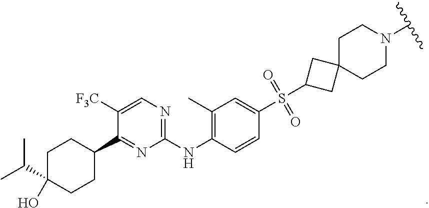 [SMILES: *N1CCC2(CC1)CC(S(=O)(=O)c1ccc(Nc3ncc(C(F)(F)F)c([C@H]4CC[C@](O)(C(C)C)CC4)n3)c(C)c1)C2]

In some embodiments, CBM is

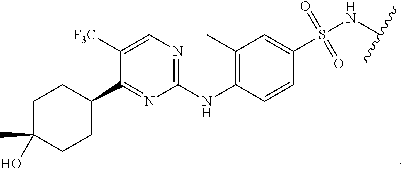 [SMILES: *NS(=O)(=O)c1ccc(Nc2ncc(C(F)(F)F)c([C@H]3CC[C@](C)(O)CC3)n2)c(C)c1]

In some embodiments, CBM is

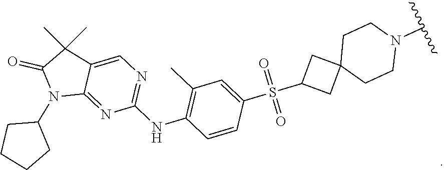 [SMILES: *N1CCC2(CC1)CC(S(=O)(=O)c1ccc(Nc3ncc4c(n3)N(C3CCCC3)C(=O)C4(C)C)c(C)c1)C2]

In some embodiments, CBM is

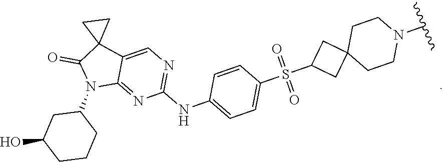

In some embodiments, CBM is

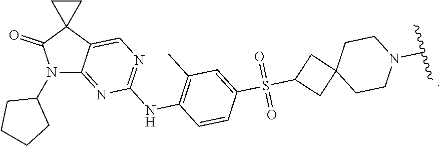 [SMILES: *N1CCC2(CC1)CC(S(=O)(=O)c1ccc(Nc3ncc4c(n3)N(C3CCCC3)C(=O)C43CC3)c(C)c1)C2]

In some embodiments, CBM is

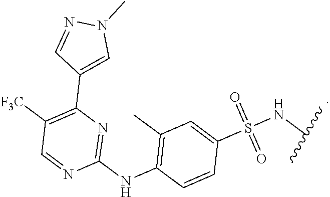 [SMILES: *NS(=O)(=O)c1ccc(Nc2ncc(C(F)(F)F)c(-c3cnn(C)c3)n2)c(C)c1]

In some embodiments, CBM is

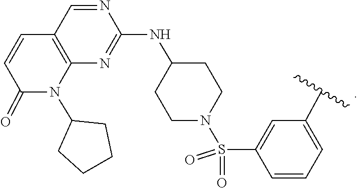 [SMILES: *c1cccc(S(=O)(=O)N2CCC(Nc3ncc4ccc(=O)n(C5CCCC5)c4n3)CC2)c1]

In some embodiments, CBM is

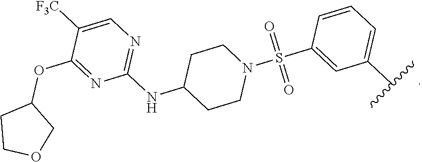 [SMILES: *c1cccc(S(=O)(=O)N2CCC(Nc3ncc(C(F)(F)F)c(OC4CCOC4)n3)CC2)c1]

In some embodiments, CBM is

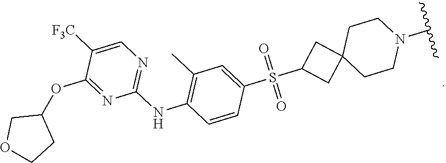 [SMILES: *N1CCC2(CC1)CC(S(=O)(=O)c1ccc(Nc3ncc(C(F)(F)F)c(OC4CCOC4)n3)c(C)c1)C2]

In some embodiments, CBM is

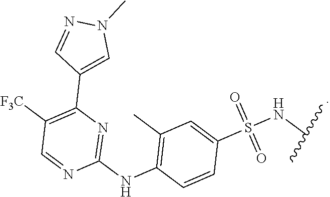 [SMILES: *NS(=O)(=O)c1ccc(Nc2ncc(C(F)(F)F)c(-c3cnn(C)c3)n2)c(C)c1]

In some embodiments, CBM is

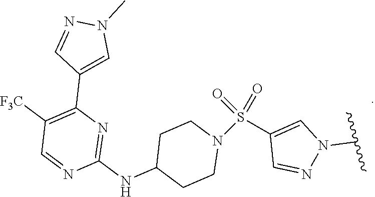 [SMILES: *n1cc(S(=O)(=O)N2CCC(Nc3ncc(C(F)(F)F)c(-c4cnn(C)c4)n3)CC2)cn1]

In some embodiments, CBM is

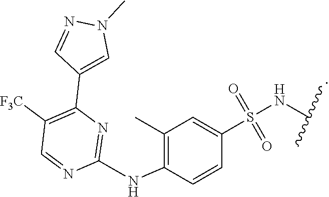 [SMILES: *NS(=O)(=O)c1ccc(Nc2ncc(C(F)(F)F)c(-c3cnn(C)c3)n2)c(C)c1]

In some embodiments, CBM is

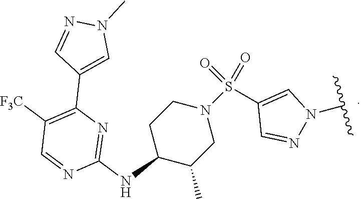 [SMILES: *n1cc(S(=O)(=O)N2CC[C@H](Nc3ncc(C(F)(F)F)c(-c4cnn(C)c4)n3)[C@@H](C)C2)cn1]

In some embodiments, CBM is

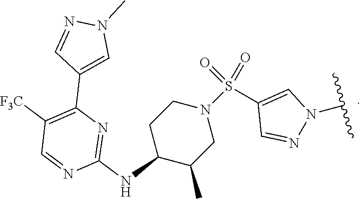 [SMILES: *n1cc(S(=O)(=O)N2CC[C@H](Nc3ncc(C(F)(F)F)c(-c4cnn(C)c4)n3)[C@H](C)C2)cn1]

In some embodiments, CBM is

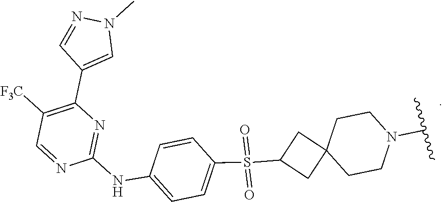 [SMILES: *N1CCC2(CC1)CC(S(=O)(=O)c1ccc(Nc3ncc(C(F)(F)F)c(-c4cnn(C)c4)n3)cc1)C2]

In some embodiments, CBM is

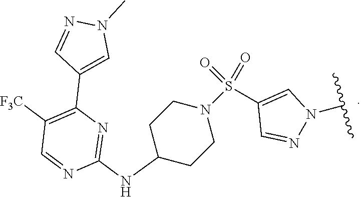 [SMILES: *n1cc(S(=O)(=O)N2CCC(Nc3ncc(C(F)(F)F)c(-c4cnn(C)c4)n3)CC2)cn1]

In some embodiments, CBM is

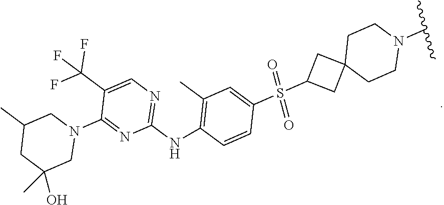 [SMILES: *N1CCC2(CC1)CC(S(=O)(=O)c1ccc(Nc3ncc(C(F)(F)F)c(N4CC(C)CC(C)(O)C4)n3)c(C)c1)C2]

In some embodiments, CBM is

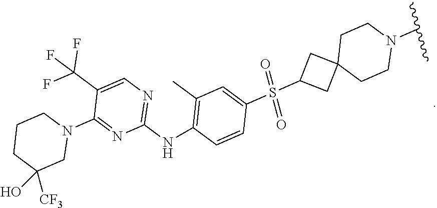 [SMILES: *N1CCC2(CC1)CC(S(=O)(=O)c1ccc(Nc3ncc(C(F)(F)F)c(N4CCCC(O)(C(F)(F)F)C4)n3)c(C)c1)C2]

In some embodiments, CBM is

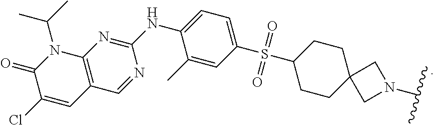 [SMILES: *N1CC2(CCC(S(=O)(=O)c3ccc(Nc4ncc5cc(Cl)c(=O)n(C(C)C)c5n4)c(C)c3)CC2)C1]

In some embodiments, CBM is

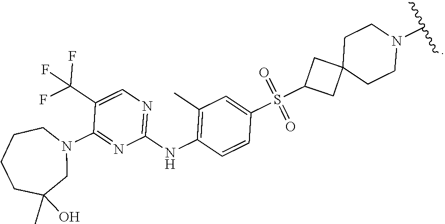 [SMILES: *N1CCC2(CC1)CC(S(=O)(=O)c1ccc(Nc3ncc(C(F)(F)F)c(N4CCCCC(C)(O)C4)n3)c(C)c1)C2]

In some embodiments, CBM is

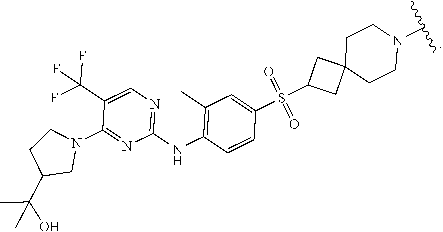 [SMILES: *N1CCC2(CC1)CC(S(=O)(=O)c1ccc(Nc3ncc(C(F)(F)F)c(N4CCC(C(C)(C)O)C4)n3)c(C)c1)C2]

In some embodiments, CBM is

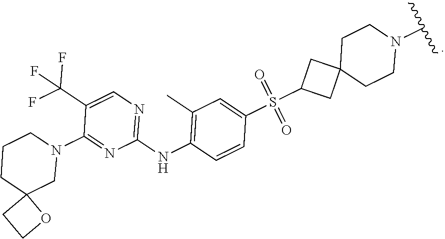 [SMILES: *N1CCC2(CC1)CC(S(=O)(=O)c1ccc(Nc3ncc(C(F)(F)F)c(N4CCCC5(CCO5)C4)n3)c(C)c1)C2]

In some embodiments, CBM is

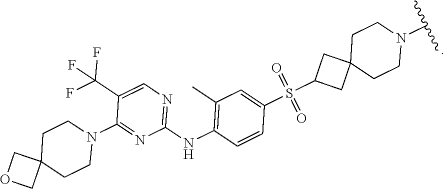 [SMILES: *N1CCC2(CC1)CC(S(=O)(=O)c1ccc(Nc3ncc(C(F)(F)F)c(N4CCC5(CC4)COC5)n3)c(C)c1)C2]

In some embodiments, CBM is

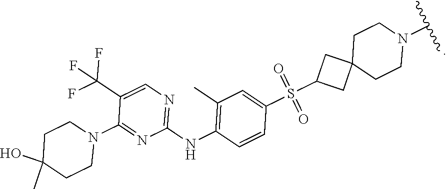 [SMILES: *N1CCC2(CC1)CC(S(=O)(=O)c1ccc(Nc3ncc(C(F)(F)F)c(N4CCC(C)(O)CC4)n3)c(C)c1)C2]

In some embodiments, CBM is

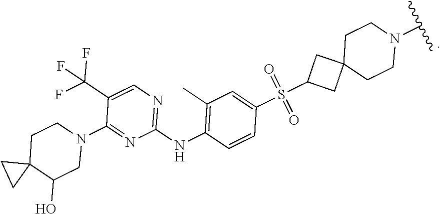 [SMILES: *N1CCC2(CC1)CC(S(=O)(=O)c1ccc(Nc3ncc(C(F)(F)F)c(N4CCC5(CC5)C(O)C4)n3)c(C)c1)C2]

In some embodiments, CBM is

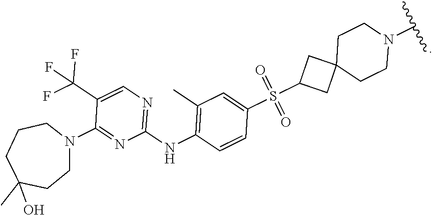 [SMILES: *N1CCC2(CC1)CC(S(=O)(=O)c1ccc(Nc3ncc(C(F)(F)F)c(N4CCCC(C)(O)CC4)n3)c(C)c1)C2]

In some embodiments, CBM is

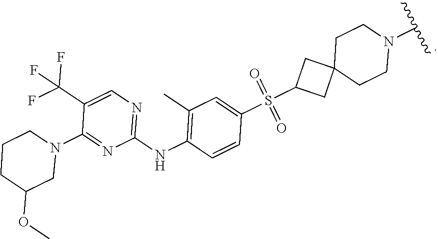 [SMILES: *N1CCC2(CC1)CC(S(=O)(=O)c1ccc(Nc3ncc(C(F)(F)F)c(N4CCCC(OC)C4)n3)c(C)c1)C2]

In some embodiments, CBM is

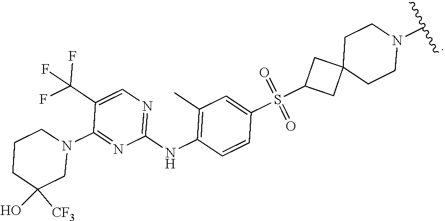 [SMILES: *N1CCC2(CC1)CC(S(=O)(=O)c1ccc(Nc3ncc(C(F)(F)F)c(N4CCCC(O)(C(F)(F)F)C4)n3)c(C)c1)C2]

In some embodiments, CBM is

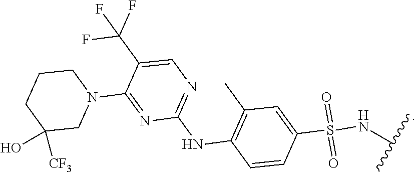 [SMILES: *NS(=O)(=O)c1ccc(Nc2ncc(C(F)(F)F)c(N3CCCC(O)(C(F)(F)F)C3)n2)c(C)c1]

In some embodiments, CBM is

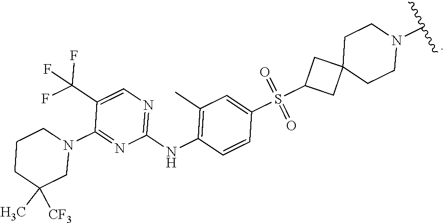 [SMILES: *N1CCC2(CC1)CC(S(=O)(=O)c1ccc(Nc3ncc(C(F)(F)F)c(N4CCCC(C)(C(F)(F)F)C4)n3)c(C)c1)C2]

In some embodiments, CBM is

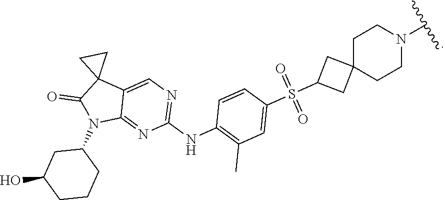 [SMILES: *N1CCC2(CC1)CC(S(=O)(=O)c1ccc(Nc3ncc4c(n3)N([C@@H]3CCC[C@@H](O)C3)C(=O)C43CC3)c(C)c1)C2]

In some embodiments, CBM is

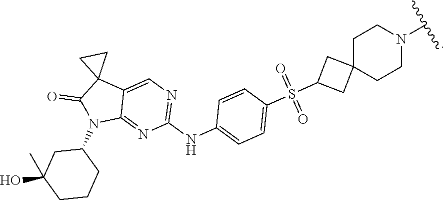 [SMILES: *N1CCC2(CC1)CC(S(=O)(=O)c1ccc(Nc3ncc4c(n3)N([C@@H]3CCC[C@@](C)(O)C3)C(=O)C43CC3)cc1)C2]

In some embodiments, CBM is

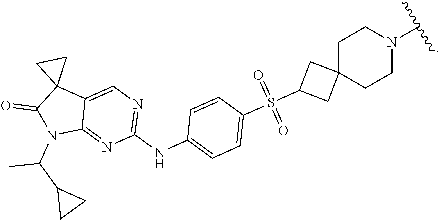 [SMILES: *N1CCC2(CC1)CC(S(=O)(=O)c1ccc(Nc3ncc4c(n3)N(C(C)C3CC3)C(=O)C43CC3)cc1)C2]

In some embodiments, CBM is

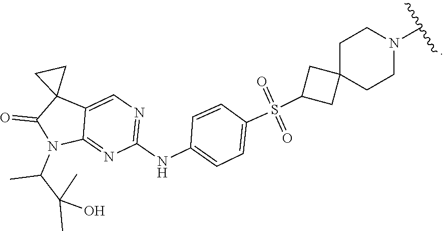 [SMILES: *N1CCC2(CC1)CC(S(=O)(=O)c1ccc(Nc3ncc4c(n3)N(C(C)C(C)(C)O)C(=O)C43CC3)cc1)C2]

In some embodiments, CBM is

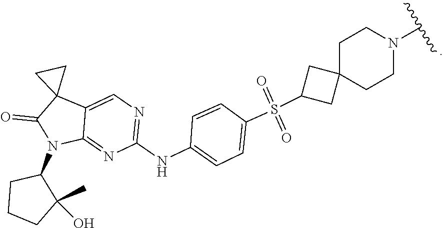 [SMILES: *N1CCC2(CC1)CC(S(=O)(=O)c1ccc(Nc3ncc4c(n3)N([C@@H]3CCC[C@@]3(C)O)C(=O)C43CC3)cc1)C2]

In some embodiments, CBM is

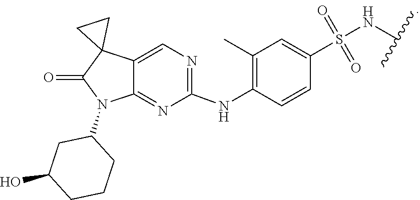

In some embodiments, CBM is

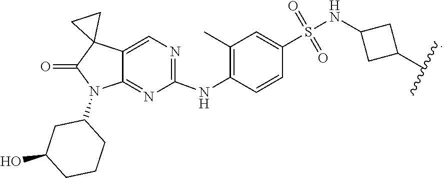

In some embodiments, CBM is

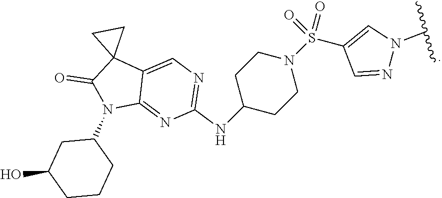

In some embodiments, CBM is

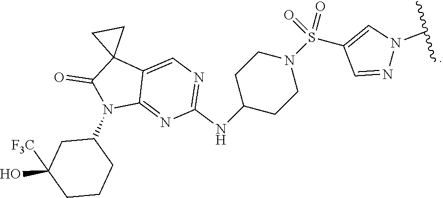

In some embodiments, CBM is

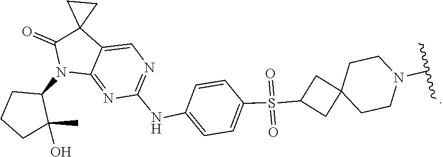 [SMILES: *N1CCC2(CC1)CC(S(=O)(=O)c1ccc(Nc3ncc4c(n3)N([C@@H]3CCC[C@@]3(C)O)C(=O)C43CC3)cc1)C2]

In some embodiments, CBM is

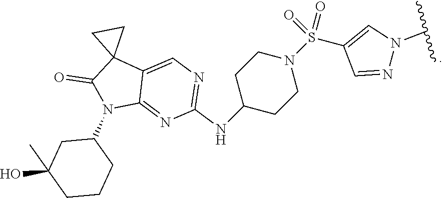

In some embodiments, CBM is

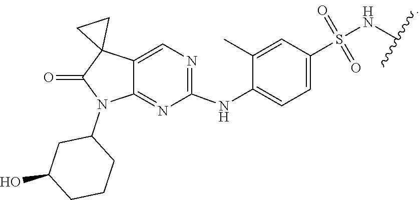 [SMILES: *NS(=O)(=O)c1ccc(Nc2ncc3c(n2)N(C2CCC[C@@H](O)C2)C(=O)C32CC2)c(C)c1]

In some embodiments, CBM is

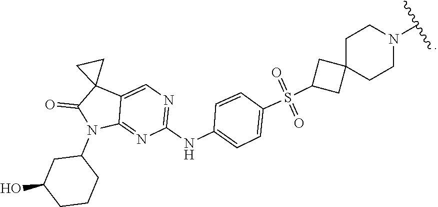 [SMILES: *N1CCC2(CC1)CC(S(=O)(=O)c1ccc(Nc3ncc4c(n3)N(C3CCC[C@@H](O)C3)C(=O)C43CC3)cc1)C2]

In some embodiments, CBM is

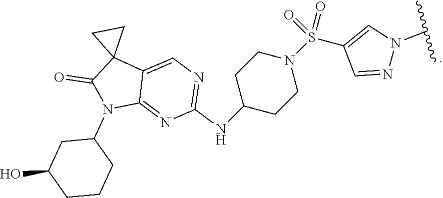 [SMILES: *n1cc(S(=O)(=O)N2CCC(Nc3ncc4c(n3)N(C3CCC[C@@H](O)C3)C(=O)C43CC3)CC2)cn1]

In some embodiments, CBM is

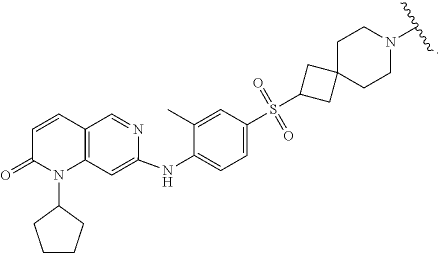 [SMILES: *N1CCC2(CC1)CC(S(=O)(=O)c1ccc(Nc3cc4c(ccc(=O)n4C4CCCC4)cn3)c(C)c1)C2]

In some embodiments, CBM is

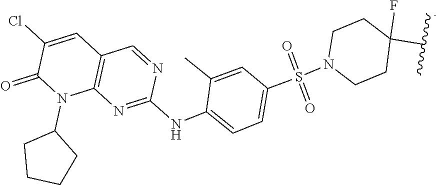 [SMILES: *C1(F)CCN(S(=O)(=O)c2ccc(Nc3ncc4cc(Cl)c(=O)n(C5CCCC5)c4n3)c(C)c2)CC1]

In some embodiments, CBM is

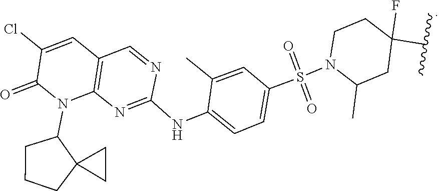 [SMILES: *C1(F)CCN(S(=O)(=O)c2ccc(Nc3ncc4cc(Cl)c(=O)n(C5CCCC56CC6)c4n3)c(C)c2)C(C)C1]

In some embodiments, CBM is

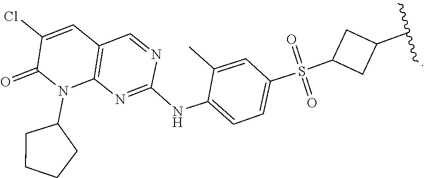 [SMILES: *C1CC(S(=O)(=O)c2ccc(Nc3ncc4cc(Cl)c(=O)n(C5CCCC5)c4n3)c(C)c2)C1]

In some embodiments, CBM is

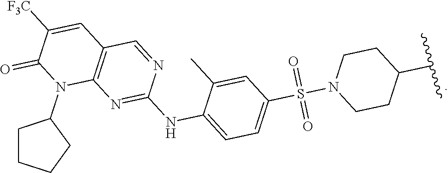 [SMILES: *C1CCN(S(=O)(=O)c2ccc(Nc3ncc4cc(C(F)(F)F)c(=O)n(C5CCCC5)c4n3)c(C)c2)CC1]

In some embodiments, CBM is

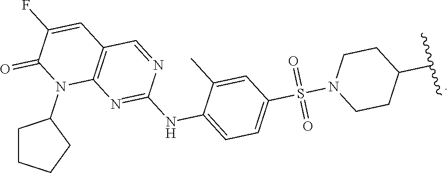 [SMILES: *C1CCN(S(=O)(=O)c2ccc(Nc3ncc4cc(F)c(=O)n(C5CCCC5)c4n3)c(C)c2)CC1]

In some embodiments, CBM is

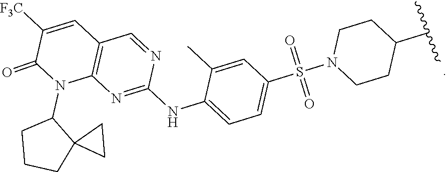 [SMILES: *C1CCN(S(=O)(=O)c2ccc(Nc3ncc4cc(C(F)(F)F)c(=O)n(C5CCCC56CC6)c4n3)c(C)c2)CC1]

In some embodiments, CBM is

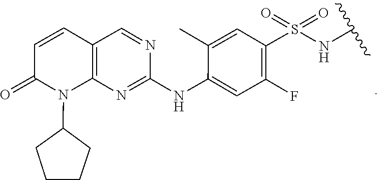 [SMILES: *NS(=O)(=O)c1cc(C)c(Nc2ncc3ccc(=O)n(C4CCCC4)c3n2)cc1F]

In some embodiments, CBM is

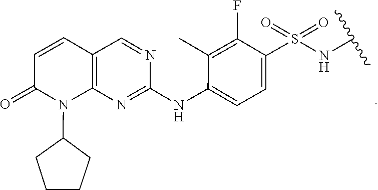 [SMILES: *NS(=O)(=O)c1ccc(Nc2ncc3ccc(=O)n(C4CCCC4)c3n2)c(C)c1F]

In some embodiments, CBM is

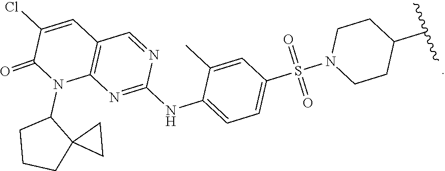 [SMILES: *C1CCN(S(=O)(=O)c2ccc(Nc3ncc4cc(Cl)c(=O)n(C5CCCC56CC6)c4n3)c(C)c2)CC1]

In some embodiments, CBM is

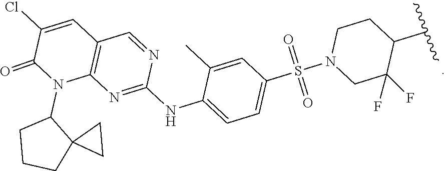 [SMILES: *C1CCN(S(=O)(=O)c2ccc(Nc3ncc4cc(Cl)c(=O)n(C5CCCC56CC6)c4n3)c(C)c2)CC1(F)F]

In some embodiments, CBM is

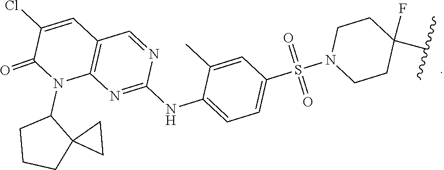 [SMILES: *C1(F)CCN(S(=O)(=O)c2ccc(Nc3ncc4cc(Cl)c(=O)n(C5CCCC56CC6)c4n3)c(C)c2)CC1]

In some embodiments, CBM is

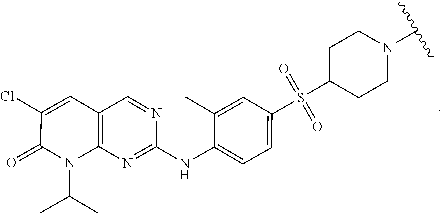 [SMILES: *N1CCC(S(=O)(=O)c2ccc(Nc3ncc4cc(Cl)c(=O)n(C(C)C)c4n3)c(C)c2)CC1]

In some embodiments, CBM is

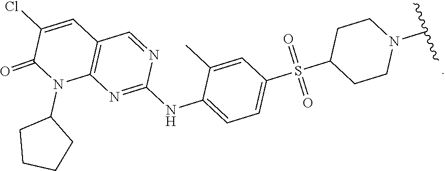 [SMILES: *N1CCC(S(=O)(=O)c2ccc(Nc3ncc4cc(Cl)c(=O)n(C5CCCC5)c4n3)c(C)c2)CC1]

In some embodiments, CBM is

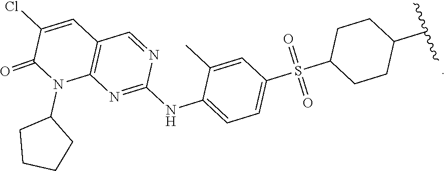 [SMILES: *C1CCC(S(=O)(=O)c2ccc(Nc3ncc4cc(Cl)c(=O)n(C5CCCC5)c4n3)c(C)c2)CC1]

In some embodiments, CBM is

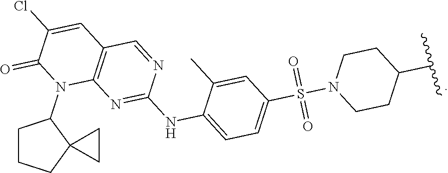 [SMILES: *C1CCN(S(=O)(=O)c2ccc(Nc3ncc4cc(Cl)c(=O)n(C5CCCC56CC6)c4n3)c(C)c2)CC1]

In some embodiments, CBM is

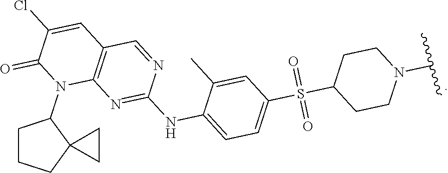 [SMILES: *N1CCC(S(=O)(=O)c2ccc(Nc3ncc4cc(Cl)c(=O)n(C5CCCC56CC6)c4n3)c(C)c2)CC1]

In some embodiments, CBM is

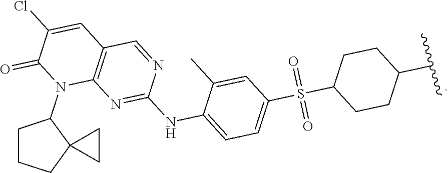 [SMILES: *C1CCC(S(=O)(=O)c2ccc(Nc3ncc4cc(Cl)c(=O)n(C5CCCC56CC6)c4n3)c(C)c2)CC1]

In some embodiments, CBM is

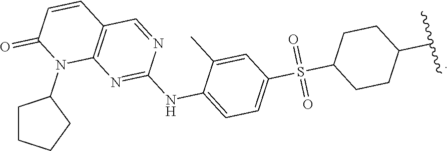 [SMILES: *C1CCC(S(=O)(=O)c2ccc(Nc3ncc4ccc(=O)n(C5CCCC5)c4n3)c(C)c2)CC1]

In some embodiments, CBM is

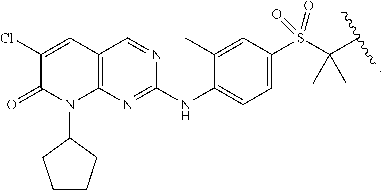 [SMILES: *C(C)(C)S(=O)(=O)c1ccc(Nc2ncc3cc(Cl)c(=O)n(C4CCCC4)c3n2)c(C)c1]

In some embodiments, CBM is

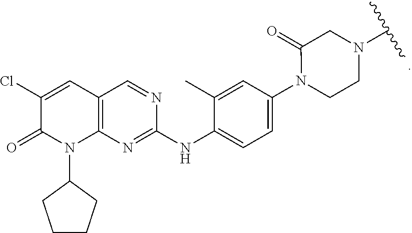 [SMILES: *N1CCN(c2ccc(Nc3ncc4cc(Cl)c(=O)n(C5CCCC5)c4n3)c(C)c2)C(=O)C1]

In some embodiments, CBM is

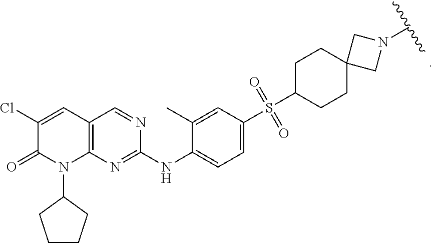 [SMILES: *N1CC2(CCC(S(=O)(=O)c3ccc(Nc4ncc5cc(Cl)c(=O)n(C6CCCC6)c5n4)c(C)c3)CC2)C1]

In some embodiments, CBM is

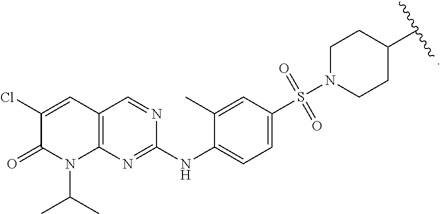 [SMILES: *C1CCN(S(=O)(=O)c2ccc(Nc3ncc4cc(Cl)c(=O)n(C(C)C)c4n3)c(C)c2)CC1]

In some embodiments, CBM is

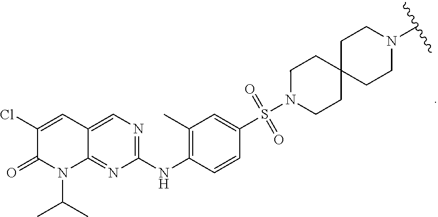 [SMILES: *N1CCC2(CC1)CCN(S(=O)(=O)c1ccc(Nc3ncc4cc(Cl)c(=O)n(C(C)C)c4n3)c(C)c1)CC2]

In some embodiments, CBM is

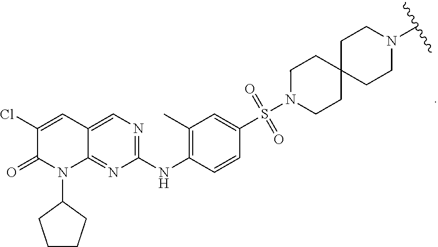 [SMILES: *N1CCC2(CC1)CCN(S(=O)(=O)c1ccc(Nc3ncc4cc(Cl)c(=O)n(C5CCCC5)c4n3)c(C)c1)CC2]

In some embodiments, CBM is

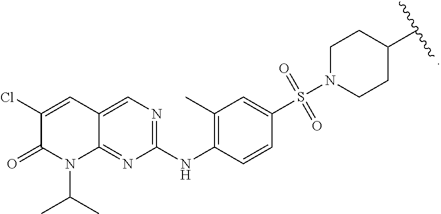 [SMILES: *C1CCN(S(=O)(=O)c2ccc(Nc3ncc4cc(Cl)c(=O)n(C(C)C)c4n3)c(C)c2)CC1]

In some embodiments, CBM is

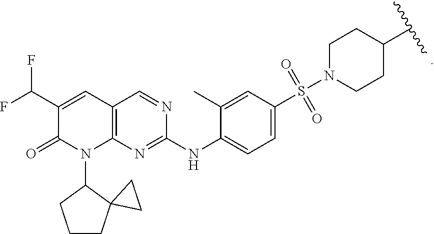 [SMILES: *C1CCN(S(=O)(=O)c2ccc(Nc3ncc4cc(C(F)F)c(=O)n(C5CCCC56CC6)c4n3)c(C)c2)CC1]

In some embodiments, CBM is

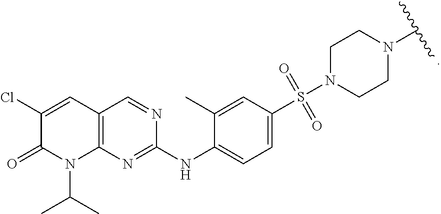 [SMILES: *N1CCN(S(=O)(=O)c2ccc(Nc3ncc4cc(Cl)c(=O)n(C(C)C)c4n3)c(C)c2)CC1]

In some embodiments, CBM is

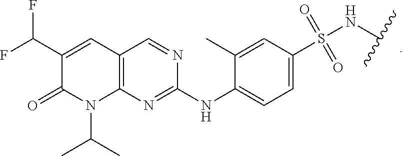 [SMILES: *NS(=O)(=O)c1ccc(Nc2ncc3cc(C(F)F)c(=O)n(C(C)C)c3n2)c(C)c1]

In some embodiments, CBM is

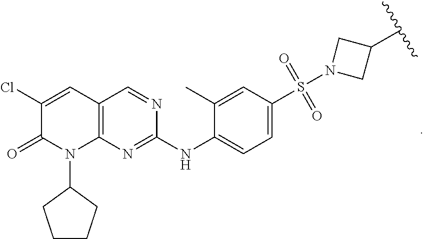 [SMILES: *C1CN(S(=O)(=O)c2ccc(Nc3ncc4cc(Cl)c(=O)n(C5CCCC5)c4n3)c(C)c2)C1]

In some embodiments, CBM is

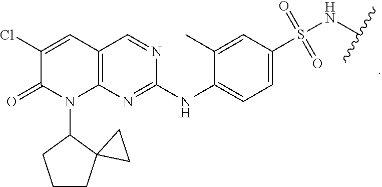 [SMILES: *NS(=O)(=O)c1ccc(Nc2ncc3cc(Cl)c(=O)n(C4CCCC45CC5)c3n2)c(C)c1]

In some embodiments, CBM is

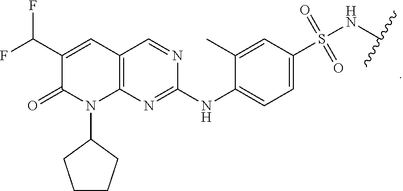 [SMILES: *NS(=O)(=O)c1ccc(Nc2ncc3cc(C(F)F)c(=O)n(C4CCCC4)c3n2)c(C)c1]

In some embodiments, CBM is

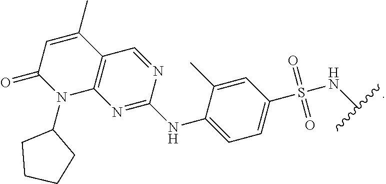 [SMILES: *NS(=O)(=O)c1ccc(Nc2ncc3c(C)cc(=O)n(C4CCCC4)c3n2)c(C)c1]

In some embodiments, CBM is

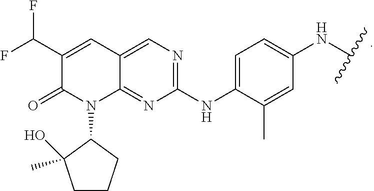 [SMILES: *Nc1ccc(Nc2ncc3cc(C(F)F)c(=O)n([C@@H]4CCC[C@@]4(C)O)c3n2)c(C)c1]

In some embodiments, CBM is

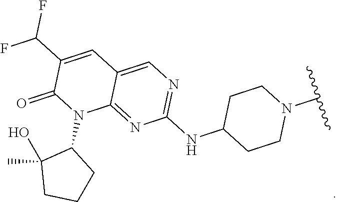 [SMILES: *N1CCC(Nc2ncc3cc(C(F)F)c(=O)n([C@@H]4CCC[C@@]4(C)O)c3n2)CC1]

In some embodiments, CBM is

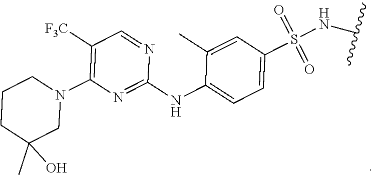 [SMILES: *NS(=O)(=O)c1ccc(Nc2ncc(C(F)(F)F)c(N3CCCC(C)(O)C3)n2)c(C)c1]

In some embodiments, CBM is

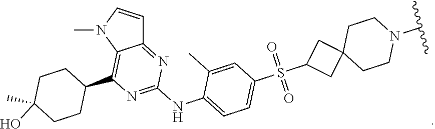 [SMILES: *N1CCC2(CC1)CC(S(=O)(=O)c1ccc(Nc3nc4ccn(C)c4c([C@H]4CC[C@@](C)(O)CC4)n3)c(C)c1)C2]

In some embodiments, CBM is

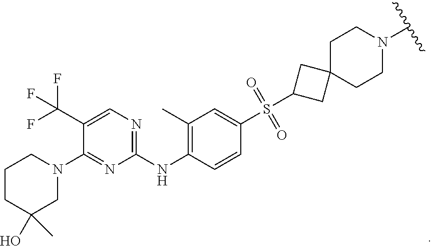 [SMILES: *N1CCC2(CC1)CC(S(=O)(=O)c1ccc(Nc3ncc(C(F)(F)F)c(N4CCCC(C)(O)C4)n3)c(C)c1)C2]

In some embodiments, CBM is

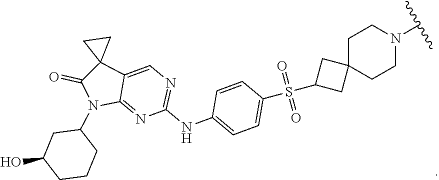 [SMILES: *N1CCC2(CC1)CC(S(=O)(=O)c1ccc(Nc3ncc4c(n3)N(C3CCC[C@@H](O)C3)C(=O)C43CC3)cc1)C2]

In some embodiments, CBM is

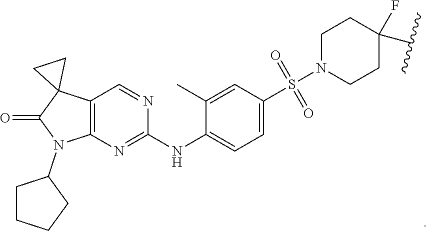 [SMILES: *C1(F)CCN(S(=O)(=O)c2ccc(Nc3ncc4c(n3)N(C3CCCC3)C(=O)C43CC3)c(C)c2)CC1]

In some embodiments, CBM is

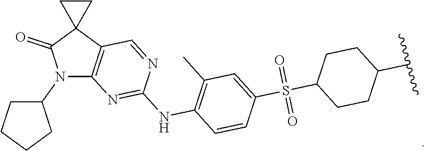 [SMILES: *C1CCC(S(=O)(=O)c2ccc(Nc3ncc4c(n3)N(C3CCCC3)C(=O)C43CC3)c(C)c2)CC1]

In some embodiments, CBM is

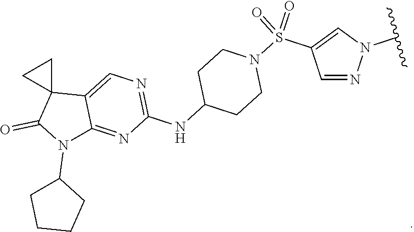 [SMILES: *n1cc(S(=O)(=O)N2CCC(Nc3ncc4c(n3)N(C3CCCC3)C(=O)C43CC3)CC2)cn1]

In some embodiments, CBM is

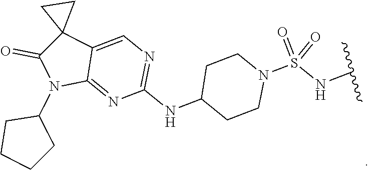 [SMILES: *NS(=O)(=O)N1CCC(Nc2ncc3c(n2)N(C2CCCC2)C(=O)C32CC2)CC1]

In some embodiments, CBM is

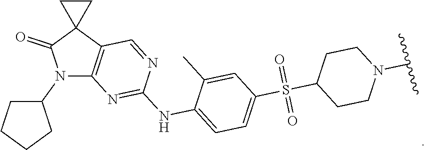 [SMILES: *N1CCC(S(=O)(=O)c2ccc(Nc3ncc4c(n3)N(C3CCCC3)C(=O)C43CC3)c(C)c2)CC1]

In some embodiments, CBM is

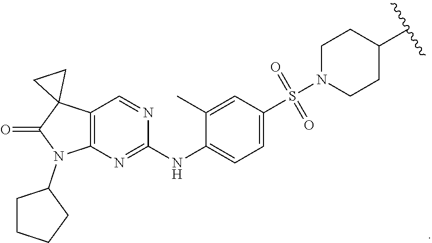 [SMILES: *C1CCN(S(=O)(=O)c2ccc(Nc3ncc4c(n3)N(C3CCCC3)C(=O)C43CC3)c(C)c2)CC1]

In some embodiments, CBM is

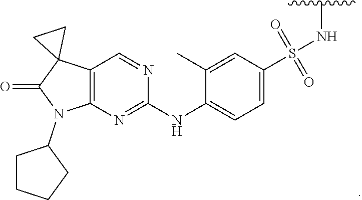 [SMILES: *NS(=O)(=O)c1ccc(Nc2ncc3c(n2)N(C2CCCC2)C(=O)C32CC2)c(C)c1]

In some embodiments, CBM is

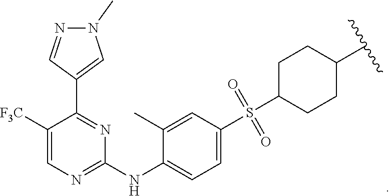 [SMILES: *C1CCC(S(=O)(=O)c2ccc(Nc3ncc(C(F)(F)F)c(-c4cnn(C)c4)n3)c(C)c2)CC1]

In some embodiments, CBM is

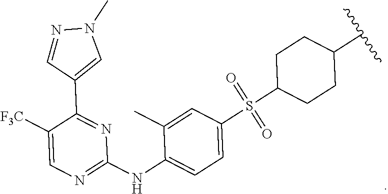 [SMILES: *C1CCC(S(=O)(=O)c2ccc(Nc3ncc(C(F)(F)F)c(-c4cnn(C)c4)n3)c(C)c2)CC1]

In some embodiments, CBM is

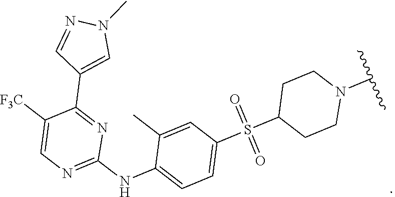 [SMILES: *N1CCC(S(=O)(=O)c2ccc(Nc3ncc(C(F)(F)F)c(-c4cnn(C)c4)n3)c(C)c2)CC1]

In some embodiments, CBM is

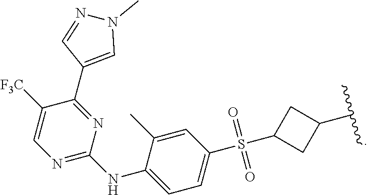 [SMILES: *C1CC(S(=O)(=O)c2ccc(Nc3ncc(C(F)(F)F)c(-c4cnn(C)c4)n3)c(C)c2)C1]

In some embodiments, CBM is

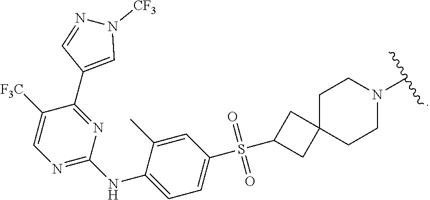 [SMILES: *N1CCC2(CC1)CC(S(=O)(=O)c1ccc(Nc3ncc(C(F)(F)F)c(-c4cnn(C(F)(F)F)c4)n3)c(C)c1)C2]

In some embodiments, CBM is

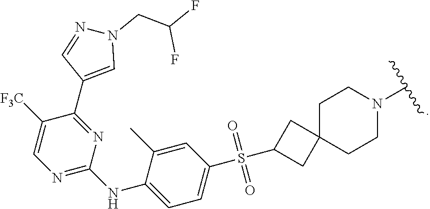 [SMILES: *N1CCC2(CC1)CC(S(=O)(=O)c1ccc(Nc3ncc(C(F)(F)F)c(-c4cnn(CC(F)F)c4)n3)c(C)c1)C2]

In some embodiments, CBM is

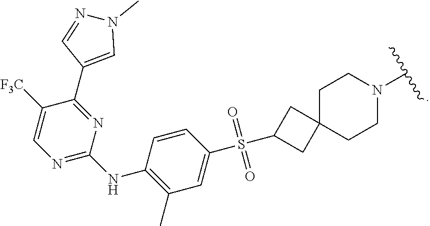 [SMILES: *N1CCC2(CC1)CC(S(=O)(=O)c1ccc(Nc3ncc(C(F)(F)F)c(-c4cnn(C)c4)n3)c(C)c1)C2]

In some embodiments, CBM is

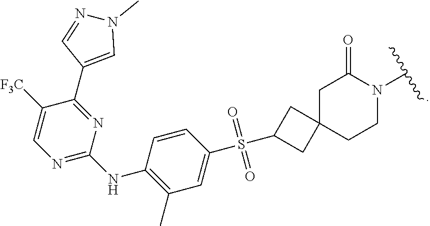 [SMILES: *N1CCC2(CC1=O)CC(S(=O)(=O)c1ccc(Nc3ncc(C(F)(F)F)c(-c4cnn(C)c4)n3)c(C)c1)C2]

In some embodiments, CBM is

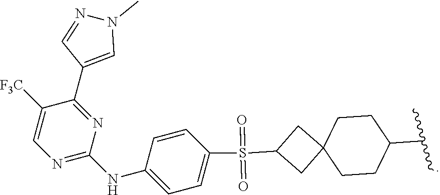 [SMILES: *C1CCC2(CC1)CC(S(=O)(=O)c1ccc(Nc3ncc(C(F)(F)F)c(-c4cnn(C)c4)n3)cc1)C2]

In some embodiments, CBM is

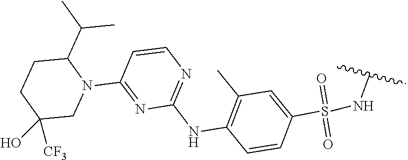 [SMILES: *NS(=O)(=O)c1ccc(Nc2nccc(N3CC(O)(C(F)(F)F)CCC3C(C)C)n2)c(C)c1]

In some embodiments, CBM is

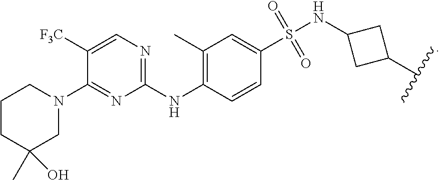 [SMILES: *C1CC(NS(=O)(=O)c2ccc(Nc3ncc(C(F)(F)F)c(N4CCCC(C)(O)C4)n3)c(C)c2)C1]

In some embodiments, CBM is

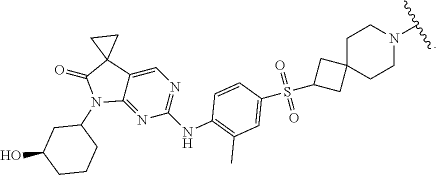 [SMILES: *N1CCC2(CC1)CC(S(=O)(=O)c1ccc(Nc3ncc4c(n3)N(C3CCC[C@@H](O)C3)C(=O)C43CC3)c(C)c1)C2]

In some embodiments, CBM is

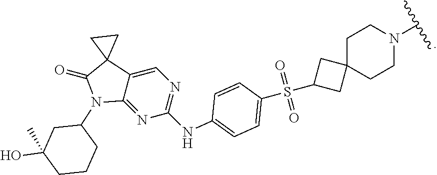 [SMILES: *N1CCC2(CC1)CC(S(=O)(=O)c1ccc(Nc3ncc4c(n3)N(C3CCC[C@@](C)(O)C3)C(=O)C43CC3)cc1)C2]

In some embodiments, CBM is

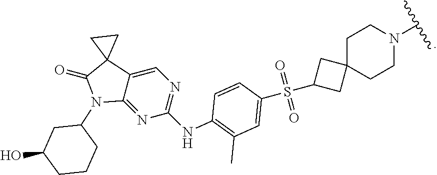 [SMILES: *N1CCC2(CC1)CC(S(=O)(=O)c1ccc(Nc3ncc4c(n3)N(C3CCC[C@@H](O)C3)C(=O)C43CC3)c(C)c1)C2]

In some embodiments, CBM is

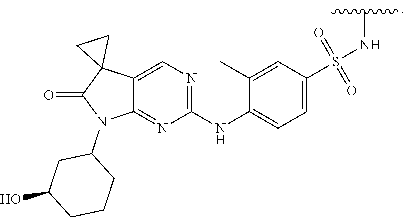 [SMILES: *NS(=O)(=O)c1ccc(Nc2ncc3c(n2)N(C2CCC[C@@H](O)C2)C(=O)C32CC2)c(C)c1]

In some embodiments, CBM is

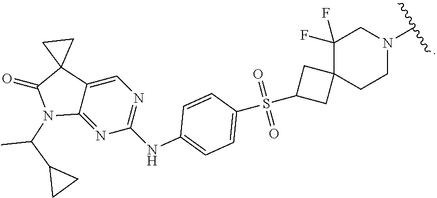 [SMILES: *N1CCC2(CC(S(=O)(=O)c3ccc(Nc4ncc5c(n4)N(C(C)C4CC4)C(=O)C54CC4)cc3)C2)C(F)(F)C1]

In some embodiments, CBM is

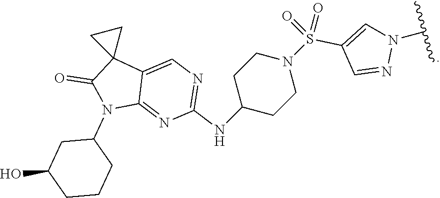 [SMILES: *n1cc(S(=O)(=O)N2CCC(Nc3ncc4c(n3)N(C3CCC[C@@H](O)C3)C(=O)C43CC3)CC2)cn1]

In some embodiments, CBM is

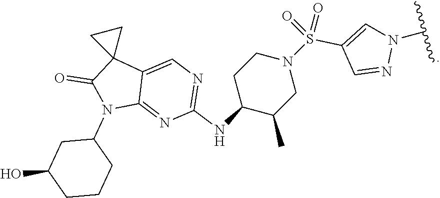 [SMILES: *n1cc(S(=O)(=O)N2CC[C@H](Nc3ncc4c(n3)N(C3CCC[C@@H](O)C3)C(=O)C43CC3)[C@H](C)C2)cn1]

In some embodiments, CBM is

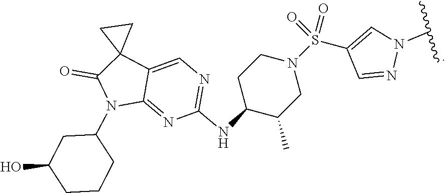 [SMILES: *n1cc(S(=O)(=O)N2CC[C@H](Nc3ncc4c(n3)N(C3CCC[C@@H](O)C3)C(=O)C43CC3)[C@@H](C)C2)cn1]

In some embodiments, CBM is

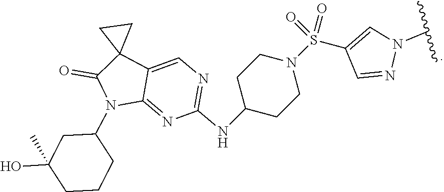 [SMILES: *n1cc(S(=O)(=O)N2CCC(Nc3ncc4c(n3)N(C3CCC[C@@](C)(O)C3)C(=O)C43CC3)CC2)cn1]

In some embodiments, CBM is

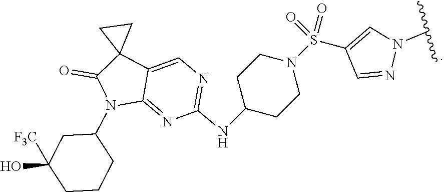 [SMILES: *n1cc(S(=O)(=O)N2CCC(Nc3ncc4c(n3)N(C3CCC[C@](O)(C(F)(F)F)C3)C(=O)C43CC3)CC2)cn1]

In some embodiments, CBM is

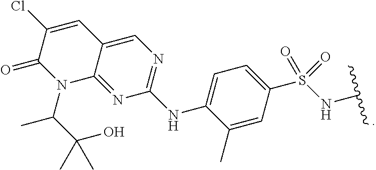 [SMILES: *NS(=O)(=O)c1ccc(Nc2ncc3cc(Cl)c(=O)n(C(C)C(C)(C)O)c3n2)c(C)c1]

In some embodiments, CBM is

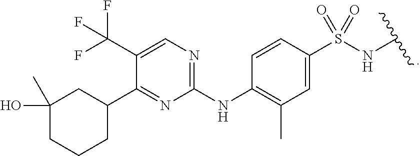 [SMILES: *NS(=O)(=O)c1ccc(Nc2ncc(C(F)(F)F)c(C3CCCC(C)(O)C3)n2)c(C)c1]

In some embodiments, CBM is

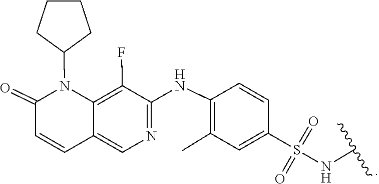 [SMILES: *NS(=O)(=O)c1ccc(Nc2ncc3ccc(=O)n(C4CCCC4)c3c2F)c(C)c1]

In some embodiments, CBM is

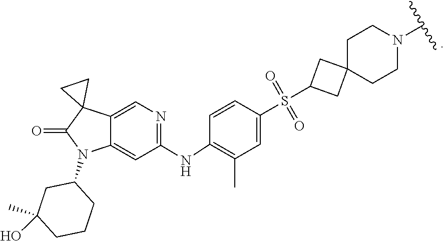 [SMILES: *N1CCC2(CC1)CC(S(=O)(=O)c1ccc(Nc3cc4c(cn3)C3(CC3)C(=O)N4[C@@H]3CCC[C@@](C)(O)C3)c(C)c1)C2]

In some embodiments, CBM is

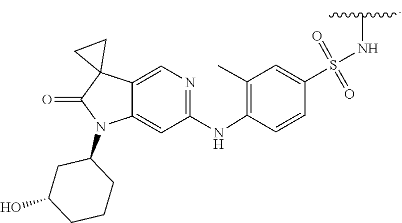 [SMILES: *NS(=O)(=O)c1ccc(Nc2cc3c(cn2)C2(CC2)C(=O)N3[C@H]2CCC[C@H](O)C2)c(C)c1]

In some embodiments, CBM is

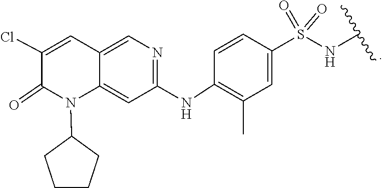 [SMILES: *NS(=O)(=O)c1ccc(Nc2cc3c(cn2)cc(Cl)c(=O)n3C2CCCC2)c(C)c1]

In some embodiments, CBM is

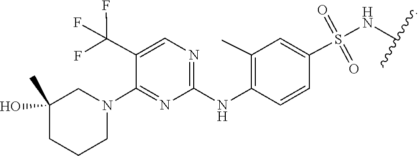 [SMILES: *NS(=O)(=O)c1ccc(Nc2ncc(C(F)(F)F)c(N3CCC[C@](C)(O)C3)n2)c(C)c1]

In some embodiments, CBM is

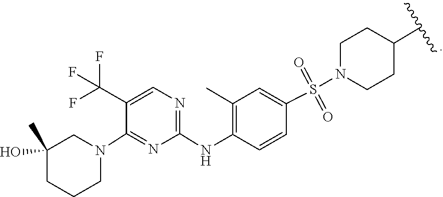 [SMILES: *C1CCN(S(=O)(=O)c2ccc(Nc3ncc(C(F)(F)F)c(N4CCC[C@](C)(O)C4)n3)c(C)c2)CC1]

In some embodiments, CBM is

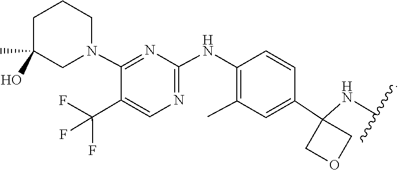 [SMILES: *NC1(c2ccc(Nc3ncc(C(F)(F)F)c(N4CCC[C@](C)(O)C4)n3)c(C)c2)COC1]

In some embodiments, CBM is

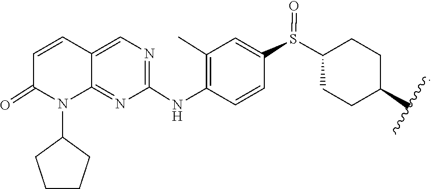 [SMILES: *[C@H]1CC[C@H]([S@](=O)c2ccc(Nc3ncc4ccc(=O)n(C5CCCC5)c4n3)c(C)c2)CC1]

In some embodiments, CBM is

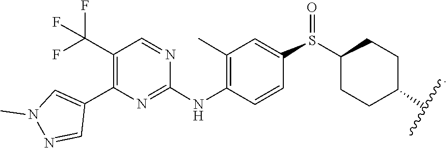 [SMILES: *[C@H]1CC[C@H]([S@](=O)c2ccc(Nc3ncc(C(F)(F)F)c(-c4cnn(C)c4)n3)c(C)c2)CC1]

In some embodiments, CBM is

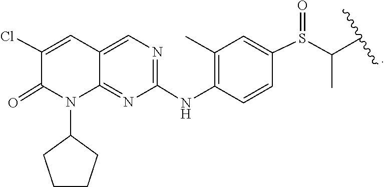 [SMILES: *C(C)S(=O)c1ccc(Nc2ncc3cc(Cl)c(=O)n(C4CCCC4)c3n2)c(C)c1]

In some embodiments, CBM is

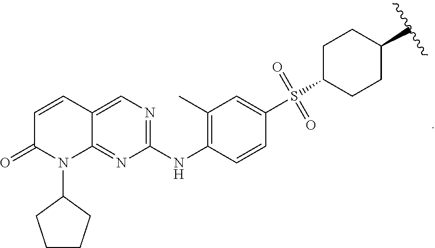 [SMILES: *[C@H]1CC[C@H](S(=O)(=O)c2ccc(Nc3ncc4ccc(=O)n(C5CCCC5)c4n3)c(C)c2)CC1]

In some embodiments, CBM is

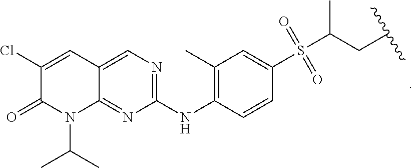 [SMILES: *CC(C)S(=O)(=O)c1ccc(Nc2ncc3cc(Cl)c(=O)n(C(C)C)c3n2)c(C)c1]

In some embodiments, CBM is

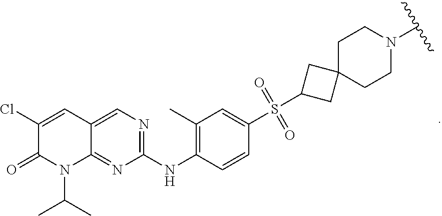 [SMILES: *N1CCC2(CC1)CC(S(=O)(=O)c1ccc(Nc3ncc4cc(Cl)c(=O)n(C(C)C)c4n3)c(C)c1)C2]

In some embodiments, CBM is

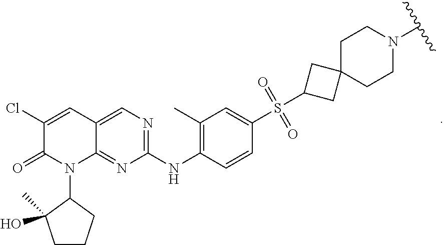 [SMILES: *N1CCC2(CC1)CC(S(=O)(=O)c1ccc(Nc3ncc4cc(Cl)c(=O)n(C5CCC[C@@]5(C)O)c4n3)c(C)c1)C2]

In some embodiments, CBM is

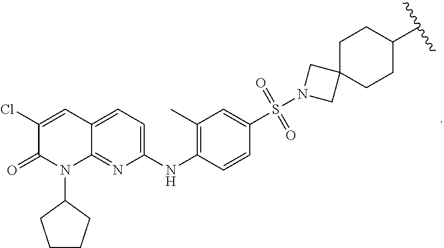 [SMILES: *C1CCC2(CC1)CN(S(=O)(=O)c1ccc(Nc3ccc4cc(Cl)c(=O)n(C5CCCC5)c4n3)c(C)c1)C2]

In some embodiments, CBM is

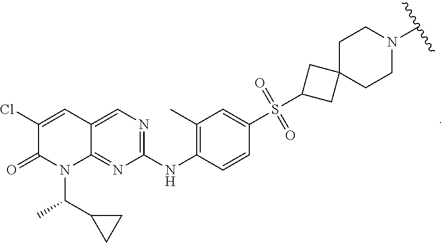 [SMILES: *N1CCC2(CC1)CC(S(=O)(=O)c1ccc(Nc3ncc4cc(Cl)c(=O)n([C@@H](C)C5CC5)c4n3)c(C)c1)C2]

In some embodiments, CBM is

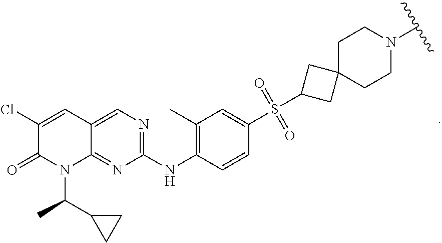 [SMILES: *N1CCC2(CC1)CC(S(=O)(=O)c1ccc(Nc3ncc4cc(Cl)c(=O)n([C@H](C)C5CC5)c4n3)c(C)c1)C2]

In some embodiments, CBM is

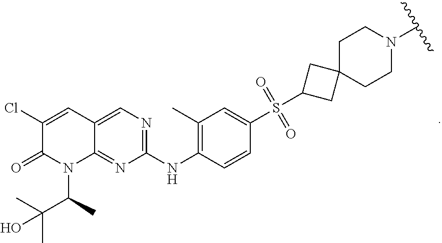 [SMILES: *N1CCC2(CC1)CC(S(=O)(=O)c1ccc(Nc3ncc4cc(Cl)c(=O)n([C@@H](C)C(C)(C)O)c4n3)c(C)c1)C2]

In some embodiments, CBM is

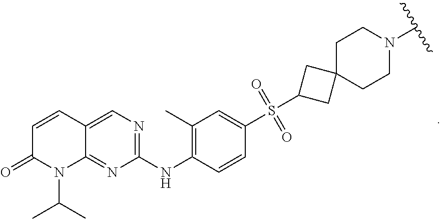 [SMILES: *N1CCC2(CC1)CC(S(=O)(=O)c1ccc(Nc3ncc4ccc(=O)n(C(C)C)c4n3)c(C)c1)C2]

In some embodiments, CBM is

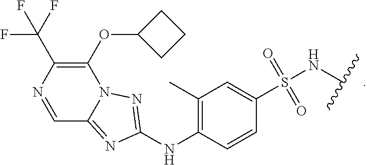 [SMILES: *NS(=O)(=O)c1ccc(Nc2nc3cnc(C(F)(F)F)c(OC4CCC4)n3n2)c(C)c1]

In some embodiments, CBM is

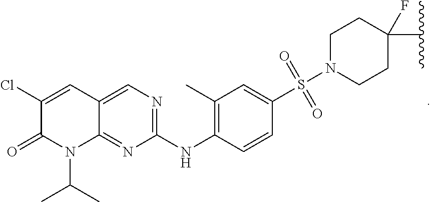 [SMILES: *C1(F)CCN(S(=O)(=O)c2ccc(Nc3ncc4cc(Cl)c(=O)n(C(C)C)c4n3)c(C)c2)CC1]

In some embodiments, CBM is

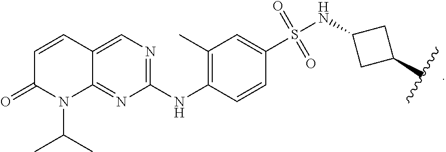 [SMILES: *[C@H]1C[C@H](NS(=O)(=O)c2ccc(Nc3ncc4ccc(=O)n(C(C)C)c4n3)c(C)c2)C1]

In some embodiments, CBM is

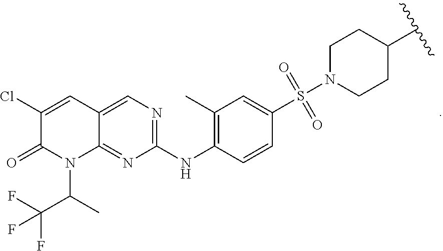 [SMILES: *C1CCN(S(=O)(=O)c2ccc(Nc3ncc4cc(Cl)c(=O)n(C(C)C(F)(F)F)c4n3)c(C)c2)CC1]

In some embodiments, CBM is

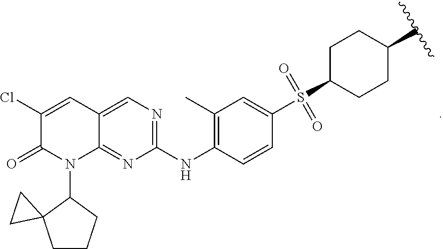 [SMILES: *[C@H]1CC[C@@H](S(=O)(=O)c2ccc(Nc3ncc4cc(Cl)c(=O)n(C5CCCC56CC6)c4n3)c(C)c2)CC1]

In some embodiments, CBM is

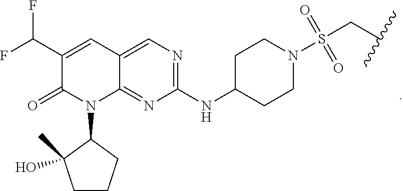 [SMILES: *CS(=O)(=O)N1CCC(Nc2ncc3cc(C(F)F)c(=O)n([C@H]4CCC[C@]4(C)O)c3n2)CC1]

In some embodiments, CBM is

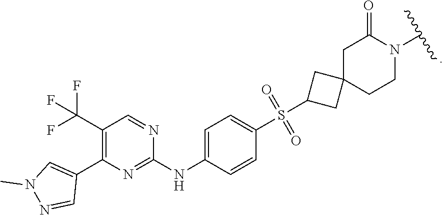 [SMILES: *N1CCC2(CC1=O)CC(S(=O)(=O)c1ccc(Nc3ncc(C(F)(F)F)c(-c4cnn(C)c4)n3)cc1)C2]

In some embodiments, CBM is

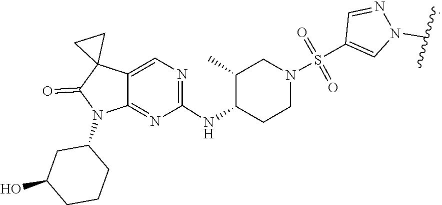 [SMILES: *n1cc(S(=O)(=O)N2CC[C@H](Nc3ncc4c(n3)N([C@@H]3CCC[C@@H](O)C3)C(=O)C43CC3)[C@H](C)C2)cn1]

In some embodiments, CBM is

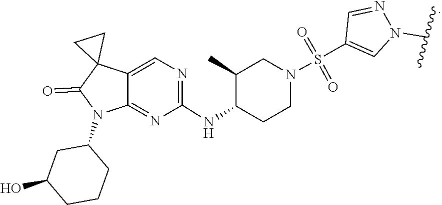 [SMILES: *n1cc(S(=O)(=O)N2CC[C@H](Nc3ncc4c(n3)N([C@@H]3CCC[C@@H](O)C3)C(=O)C43CC3)[C@@H](C)C2)cn1]

In some embodiments, CBM is

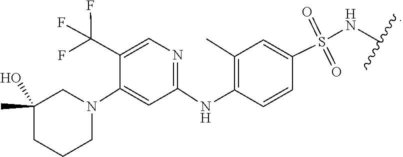 [SMILES: *NS(=O)(=O)c1ccc(Nc2cc(N3CCC[C@](C)(O)C3)c(C(F)(F)F)cn2)c(C)c1]

In some embodiments, CBM is

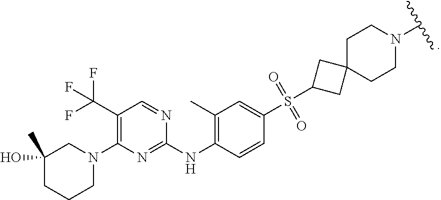 [SMILES: *N1CCC2(CC1)CC(S(=O)(=O)c1ccc(Nc3ncc(C(F)(F)F)c(N4CCC[C@](C)(O)C4)n3)c(C)c1)C2]

In some embodiments, CBM is

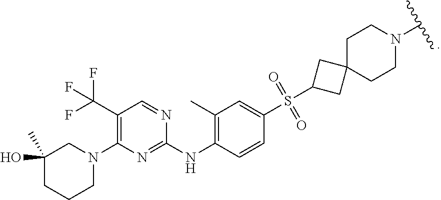 [SMILES: *N1CCC2(CC1)CC(S(=O)(=O)c1ccc(Nc3ncc(C(F)(F)F)c(N4CCC[C@@](C)(O)C4)n3)c(C)c1)C2]

In some embodiments, CBM is

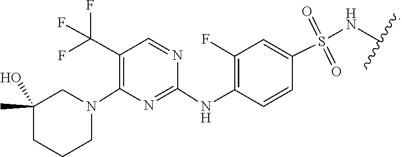 [SMILES: *NS(=O)(=O)c1ccc(Nc2ncc(C(F)(F)F)c(N3CCC[C@](C)(O)C3)n2)c(F)c1]

In some embodiments, CBM is

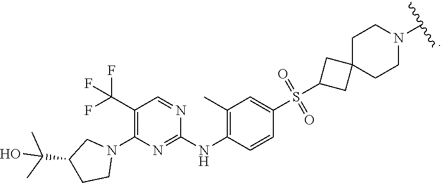 [SMILES: *N1CCC2(CC1)CC(S(=O)(=O)c1ccc(Nc3ncc(C(F)(F)F)c(N4CC[C@H](C(C)(C)O)C4)n3)c(C)c1)C2]

In some embodiments, CBM is

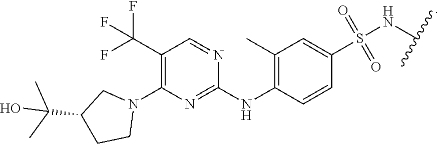 [SMILES: *NS(=O)(=O)c1ccc(Nc2ncc(C(F)(F)F)c(N3CC[C@H](C(C)(C)O)C3)n2)c(C)c1]

In some embodiments, CBM is

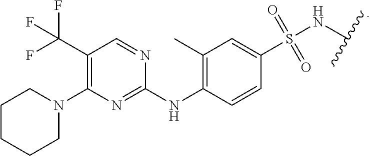 [SMILES: *NS(=O)(=O)c1ccc(Nc2ncc(C(F)(F)F)c(N3CCCCC3)n2)c(C)c1]

In some embodiments, CBM is

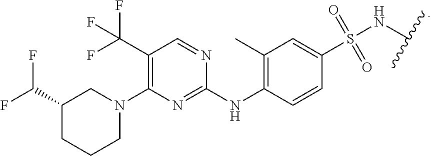 [SMILES: *NS(=O)(=O)c1ccc(Nc2ncc(C(F)(F)F)c(N3CCC[C@H](C(F)F)C3)n2)c(C)c1]

In some embodiments, CBM is

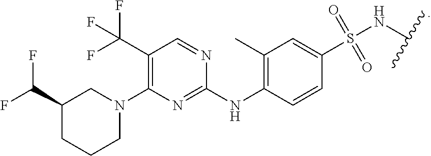 [SMILES: *NS(=O)(=O)c1ccc(Nc2ncc(C(F)(F)F)c(N3CCC[C@@H](C(F)F)C3)n2)c(C)c1]

In some embodiments, CBM is

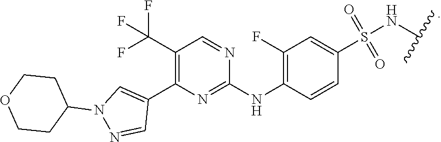 [SMILES: *NS(=O)(=O)c1ccc(Nc2ncc(C(F)(F)F)c(-c3cnn(C4CCOCC4)c3)n2)c(F)c1]

In some embodiments, CBM is

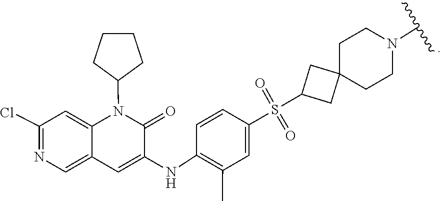 [SMILES: *N1CCC2(CC1)CC(S(=O)(=O)c1ccc(Nc3cc4cnc(Cl)cc4n(C4CCCC4)c3=O)c(C)c1)C2]

In some embodiments, CBM is

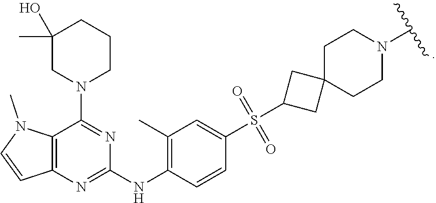 [SMILES: *N1CCC2(CC1)CC(S(=O)(=O)c1ccc(Nc3nc(N4CCCC(C)(O)C4)c4c(ccn4C)n3)c(C)c1)C2]

In some embodiments, CBM is

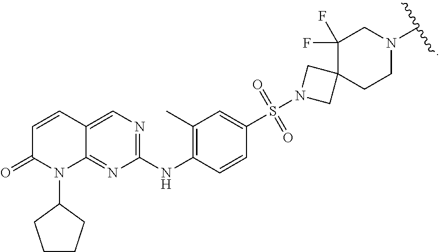 [SMILES: *N1CCC2(CN(S(=O)(=O)c3ccc(Nc4ncc5ccc(=O)n(C6CCCC6)c5n4)c(C)c3)C2)C(F)(F)C1]

In some embodiments, CBM is

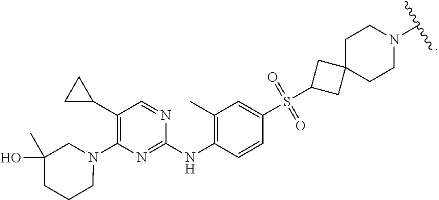 [SMILES: *N1CCC2(CC1)CC(S(=O)(=O)c1ccc(Nc3ncc(C4CC4)c(N4CCCC(C)(O)C4)n3)c(C)c1)C2]

In some embodiments, CBM is

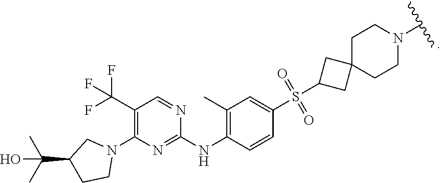 [SMILES: *N1CCC2(CC1)CC(S(=O)(=O)c1ccc(Nc3ncc(C(F)(F)F)c(N4CC[C@@H](C(C)(C)O)C4)n3)c(C)c1)C2]

In some embodiments, CBM is

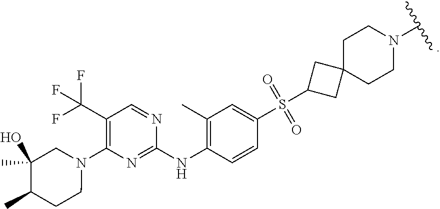 [SMILES: *N1CCC2(CC1)CC(S(=O)(=O)c1ccc(Nc3ncc(C(F)(F)F)c(N4CC[C@@H](C)[C@@](C)(O)C4)n3)c(C)c1)C2]

In some embodiments, CBM is

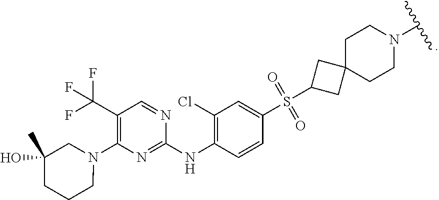 [SMILES: *N1CCC2(CC1)CC(S(=O)(=O)c1ccc(Nc3ncc(C(F)(F)F)c(N4CCC[C@](C)(O)C4)n3)c(Cl)c1)C2]

In some embodiments, CBM is

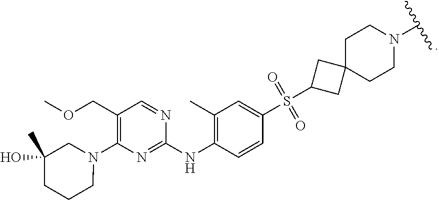 [SMILES: *N1CCC2(CC1)CC(S(=O)(=O)c1ccc(Nc3ncc(COC)c(N4CCC[C@](C)(O)C4)n3)c(C)c1)C2]

In some embodiments, CBM is

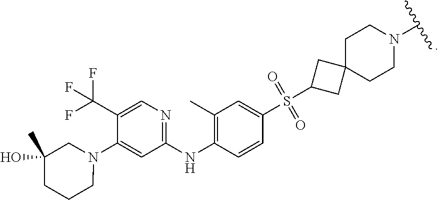 [SMILES: *N1CCC2(CC1)CC(S(=O)(=O)c1ccc(Nc3cc(N4CCC[C@](C)(O)C4)c(C(F)(F)F)cn3)c(C)c1)C2]

In some embodiments, CBM is

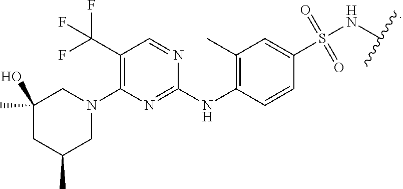 [SMILES: *NS(=O)(=O)c1ccc(Nc2ncc(C(F)(F)F)c(N3C[C@@H](C)C[C@@](C)(O)C3)n2)c(C)c1]

In some embodiments, CBM is

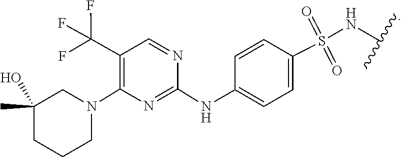 [SMILES: *NS(=O)(=O)c1ccc(Nc2ncc(C(F)(F)F)c(N3CCC[C@](C)(O)C3)n2)cc1]

In some embodiments, CBM is

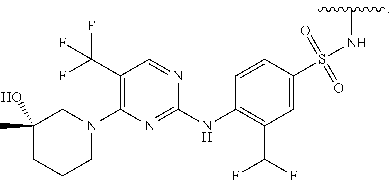 [SMILES: *NS(=O)(=O)c1ccc(Nc2ncc(C(F)(F)F)c(N3CCC[C@](C)(O)C3)n2)c(C(F)F)c1]

In some embodiments, CBM is

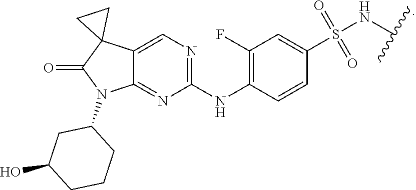 [SMILES: *NS(=O)(=O)c1ccc(Nc2ncc3c(n2)N([C@@H]2CCC[C@@H](O)C2)C(=O)C32CC2)c(F)c1]

In some embodiments, CBM is

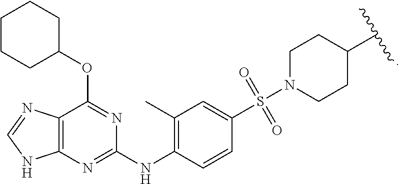 [SMILES: *C1CCN(S(=O)(=O)c2ccc(Nc3nc(OC4CCCCC4)c4nc[nH]c4n3)c(C)c2)CC1]

In some embodiments, CBM is

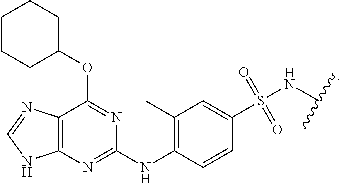 [SMILES: *NS(=O)(=O)c1ccc(Nc2nc(OC3CCCCC3)c3nc[nH]c3n2)c(C)c1]

In some embodiments, CBM is

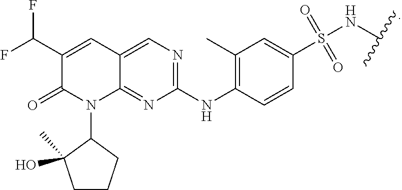 [SMILES: *NS(=O)(=O)c1ccc(Nc2ncc3cc(C(F)F)c(=O)n(C4CCC[C@@]4(C)O)c3n2)c(C)c1]

In some embodiments, CBM is

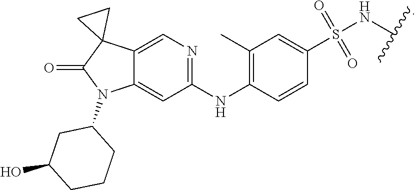 [SMILES: *NS(=O)(=O)c1ccc(Nc2cc3c(cn2)C2(CC2)C(=O)N3[C@@H]2CCC[C@@H](O)C2)c(C)c1]

In some embodiments, CBM is

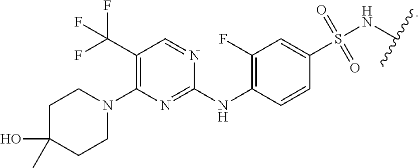 [SMILES: *NS(=O)(=O)c1ccc(Nc2ncc(C(F)(F)F)c(N3CCC(C)(O)CC3)n2)c(F)c1]

In some embodiments, CBM is

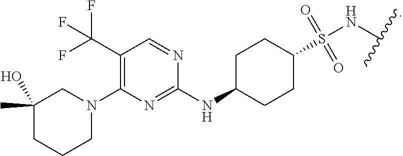 [SMILES: *NS(=O)(=O)[C@H]1CC[C@H](Nc2ncc(C(F)(F)F)c(N3CCC[C@](C)(O)C3)n2)CC1]

In some embodiments, CBM is

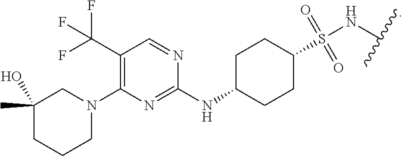 [SMILES: *NS(=O)(=O)[C@H]1CC[C@@H](Nc2ncc(C(F)(F)F)c(N3CCC[C@](C)(O)C3)n2)CC1]

In some embodiments, CBM is

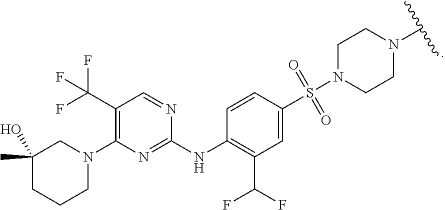 [SMILES: *N1CCN(S(=O)(=O)c2ccc(Nc3ncc(C(F)(F)F)c(N4CCC[C@](C)(O)C4)n3)c(C(F)F)c2)CC1]

In some embodiments, CBM is

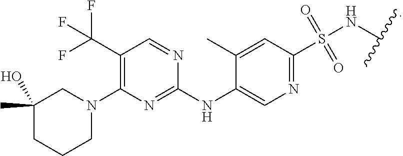 [SMILES: *NS(=O)(=O)c1cc(C)c(Nc2ncc(C(F)(F)F)c(N3CCC[C@](C)(O)C3)n2)cn1]

In some embodiments, CBM is

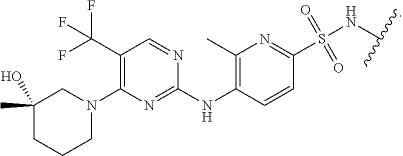 [SMILES: *NS(=O)(=O)c1ccc(Nc2ncc(C(F)(F)F)c(N3CCC[C@](C)(O)C3)n2)c(C)n1]

In some embodiments, CBM is

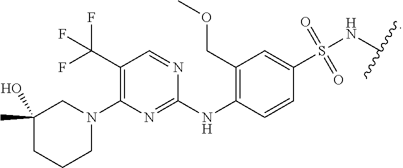 [SMILES: *NS(=O)(=O)c1ccc(Nc2ncc(C(F)(F)F)c(N3CCC[C@](C)(O)C3)n2)c(COC)c1]

In some embodiments, CBM is

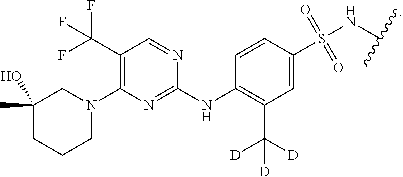 [SMILES: *NS(=O)(=O)c1ccc(Nc2ncc(C(F)(F)F)c(N3CCC[C@](C)(O)C3)n2)c(C([2H])([2H])[2H])c1]

In some embodiments, CBM is

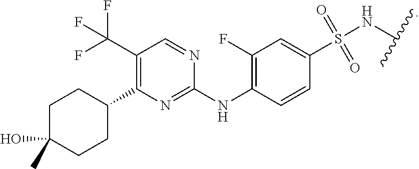 [SMILES: *NS(=O)(=O)c1ccc(Nc2ncc(C(F)(F)F)c([C@H]3CC[C@@](C)(O)CC3)n2)c(F)c1]

In some embodiments, CBM is

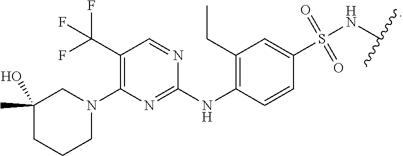 [SMILES: *NS(=O)(=O)c1ccc(Nc2ncc(C(F)(F)F)c(N3CCC[C@](C)(O)C3)n2)c(CC)c1]

In some embodiments, CBM is

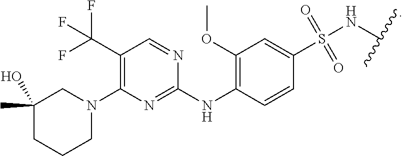 [SMILES: *NS(=O)(=O)c1ccc(Nc2ncc(C(F)(F)F)c(N3CCC[C@](C)(O)C3)n2)c(OC)c1]

In some embodiments, CBM is

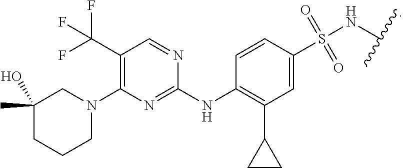 [SMILES: *NS(=O)(=O)c1ccc(Nc2ncc(C(F)(F)F)c(N3CCC[C@](C)(O)C3)n2)c(C2CC2)c1]

In some embodiments, CBM is

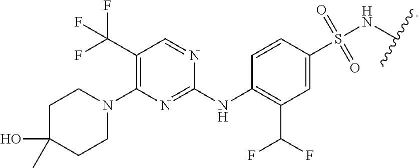 [SMILES: *NS(=O)(=O)c1ccc(Nc2ncc(C(F)(F)F)c(N3CCC(C)(O)CC3)n2)c(C(F)F)c1]

In some embodiments, CBM is

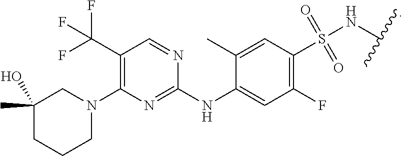 [SMILES: *NS(=O)(=O)c1cc(C)c(Nc2ncc(C(F)(F)F)c(N3CCC[C@](C)(O)C3)n2)cc1F]

In some embodiments, CBM is

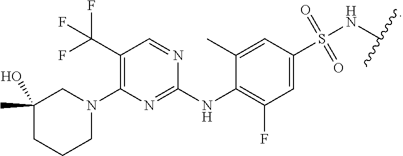 [SMILES: *NS(=O)(=O)c1cc(C)c(Nc2ncc(C(F)(F)F)c(N3CCC[C@](C)(O)C3)n2)c(F)c1]

In some embodiments, CBM is

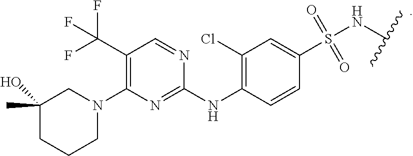 [SMILES: *NS(=O)(=O)c1ccc(Nc2ncc(C(F)(F)F)c(N3CCC[C@](C)(O)C3)n2)c(Cl)c1]

In some embodiments, CBM is

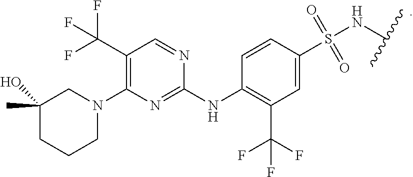 [SMILES: *NS(=O)(=O)c1ccc(Nc2ncc(C(F)(F)F)c(N3CCC[C@](C)(O)C3)n2)c(C(F)(F)F)c1]

In some embodiments, CBM is

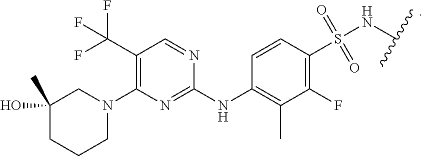 [SMILES: *NS(=O)(=O)c1ccc(Nc2ncc(C(F)(F)F)c(N3CCC[C@](C)(O)C3)n2)c(C)c1F]

In some embodiments, CBM is

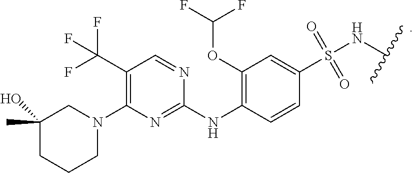 [SMILES: *NS(=O)(=O)c1ccc(Nc2ncc(C(F)(F)F)c(N3CCC[C@](C)(O)C3)n2)c(OC(F)F)c1]

In some embodiments, CBM is

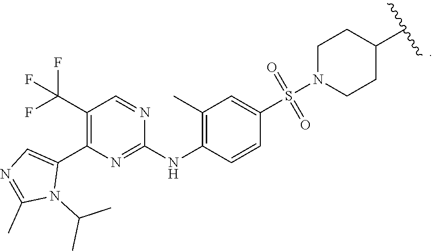 [SMILES: *C1CCN(S(=O)(=O)c2ccc(Nc3ncc(C(F)(F)F)c(-c4cnc(C)n4C(C)C)n3)c(C)c2)CC1]

In some embodiments, CBM is

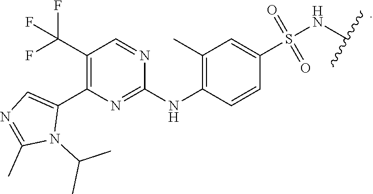 [SMILES: *NS(=O)(=O)c1ccc(Nc2ncc(C(F)(F)F)c(-c3cnc(C)n3C(C)C)n2)c(C)c1]

In some embodiments, CBM is

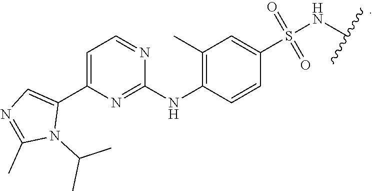 [SMILES: *NS(=O)(=O)c1ccc(Nc2nccc(-c3cnc(C)n3C(C)C)n2)c(C)c1]

In some embodiments, CBM is

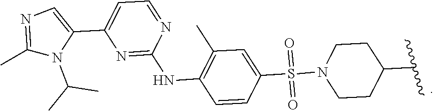 [SMILES: *C1CCN(S(=O)(=O)c2ccc(Nc3nccc(-c4cnc(C)n4C(C)C)n3)c(C)c2)CC1]

In some embodiments, CBM is

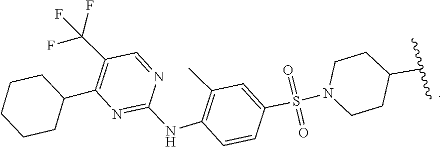 [SMILES: *C1CCN(S(=O)(=O)c2ccc(Nc3ncc(C(F)(F)F)c(C4CCCCC4)n3)c(C)c2)CC1]

In some embodiments, CBM is

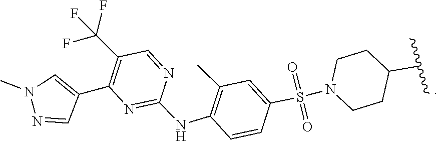 [SMILES: *C1CCN(S(=O)(=O)c2ccc(Nc3ncc(C(F)(F)F)c(-c4cnn(C)c4)n3)c(C)c2)CC1]

In some embodiments, CBM is

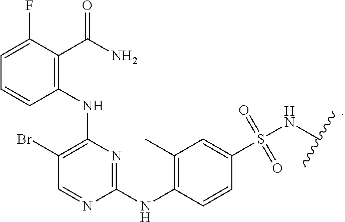 [SMILES: *NS(=O)(=O)c1ccc(Nc2ncc(Br)c(Nc3cccc(F)c3C(N)=O)n2)c(C)c1]

In some embodiments, CBM is

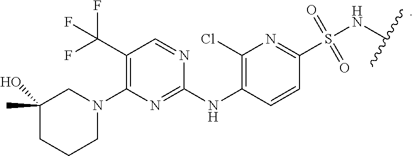 [SMILES: *NS(=O)(=O)c1ccc(Nc2ncc(C(F)(F)F)c(N3CCC[C@](C)(O)C3)n2)c(Cl)n1]

In some embodiments, CBM is

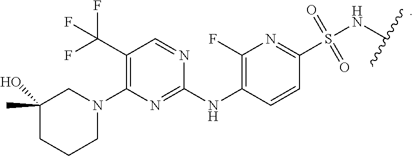 [SMILES: *NS(=O)(=O)c1ccc(Nc2ncc(C(F)(F)F)c(N3CCC[C@](C)(O)C3)n2)c(F)n1]

In some embodiments, CBM is

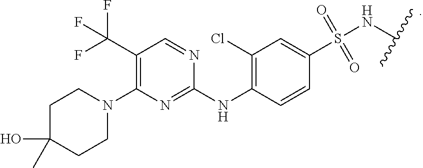 [SMILES: *NS(=O)(=O)c1ccc(Nc2ncc(C(F)(F)F)c(N3CCC(C)(O)CC3)n2)c(Cl)c1]

In some embodiments, CBM is

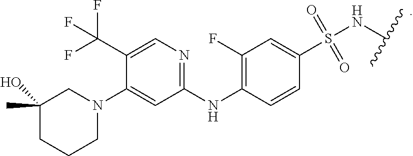 [SMILES: *NS(=O)(=O)c1ccc(Nc2cc(N3CCC[C@](C)(O)C3)c(C(F)(F)F)cn2)c(F)c1]

In some embodiments, CBM is

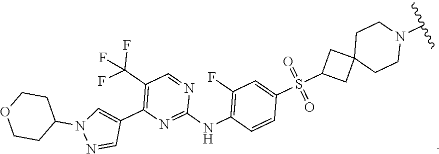 [SMILES: *N1CCC2(CC1)CC(S(=O)(=O)c1ccc(Nc3ncc(C(F)(F)F)c(-c4cnn(C5CCOCC5)c4)n3)c(F)c1)C2]

In some embodiments, CBM is

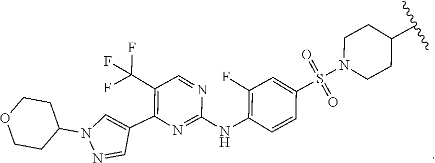 [SMILES: *C1CCN(S(=O)(=O)c2ccc(Nc3ncc(C(F)(F)F)c(-c4cnn(C5CCOCC5)c4)n3)c(F)c2)CC1]

In some embodiments, CBM is

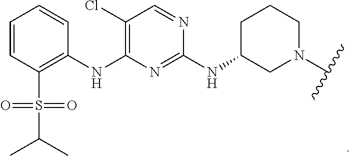 [SMILES: *N1CCC[C@@H](Nc2ncc(Cl)c(Nc3ccccc3S(=O)(=O)C(C)C)n2)C1]

In some embodiments, CBM is

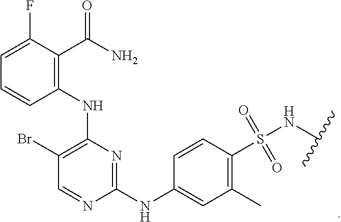 [SMILES: *NS(=O)(=O)c1ccc(Nc2ncc(Br)c(Nc3cccc(F)c3C(N)=O)n2)cc1C]

In some embodiments, CBM is

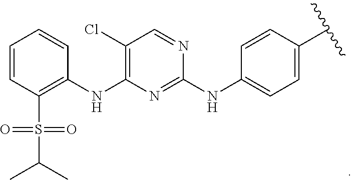 [SMILES: *c1ccc(Nc2ncc(Cl)c(Nc3ccccc3S(=O)(=O)C(C)C)n2)cc1]

In some embodiments, CBM is

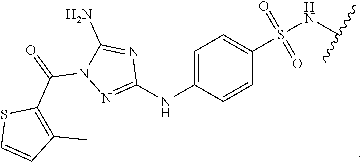 [SMILES: *NS(=O)(=O)c1ccc(Nc2nc(N)n(C(=O)c3sccc3C)n2)cc1]

In some embodiments, CBM is

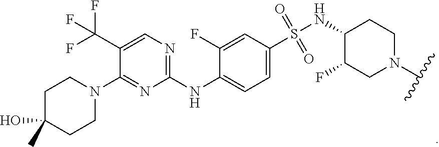

In some embodiments, CBM is

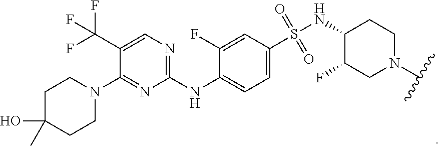 [SMILES: *N1CC[C@@H](NS(=O)(=O)c2ccc(Nc3ncc(C(F)(F)F)c(N4CCC(C)(O)CC4)n3)c(F)c2)[C@@H](F)C1]

In some embodiments, CBM is

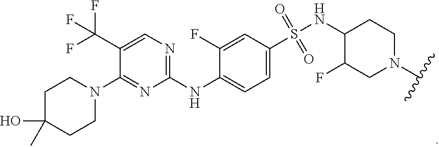 [SMILES: *N1CCC(NS(=O)(=O)c2ccc(Nc3ncc(C(F)(F)F)c(N4CCC(C)(O)CC4)n3)c(F)c2)C(F)C1]

In some embodiments, CBM is

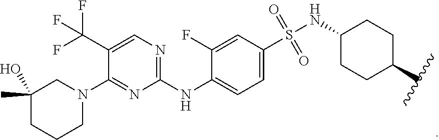 [SMILES: *[C@H]1CC[C@H](NS(=O)(=O)c2ccc(Nc3ncc(C(F)(F)F)c(N4CCC[C@](C)(O)C4)n3)c(F)c2)CC1]

In some embodiments, CBM is

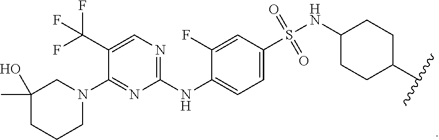 [SMILES: *C1CCC(NS(=O)(=O)c2ccc(Nc3ncc(C(F)(F)F)c(N4CCCC(C)(O)C4)n3)c(F)c2)CC1]

In some embodiments, CBM is

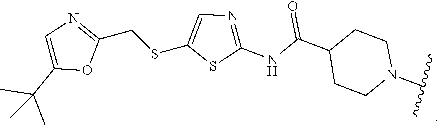 [SMILES: *N1CCC(C(=O)Nc2ncc(SCc3ncc(C(C)(C)C)o3)s2)CC1]

In some embodiments, CBM is

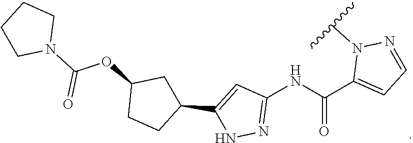 [SMILES: *n1nccc1C(=O)Nc1cc([C@H]2CC[C@@H](OC(=O)N3CCCC3)C2)[nH]n1]

In some embodiments, CBM is

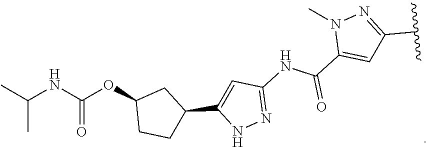 [SMILES: *c1cc(C(=O)Nc2cc([C@H]3CC[C@@H](OC(=O)NC(C)C)C3)[nH]n2)n(C)n1]

In some embodiments, CBM is

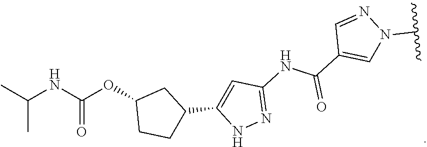

In some embodiments, CBM is

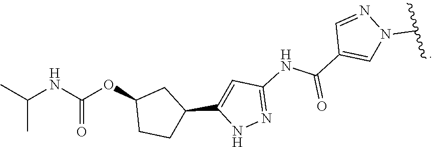 [SMILES: *n1cc(C(=O)Nc2cc([C@H]3CC[C@@H](OC(=O)NC(C)C)C3)[nH]n2)cn1]

In some embodiments, CBM is

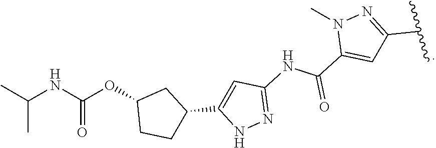 [SMILES: *c1cc(C(=O)Nc2cc([C@@H]3CC[C@H](OC(=O)NC(C)C)C3)[nH]n2)n(C)n1]

In some embodiments, CBM is

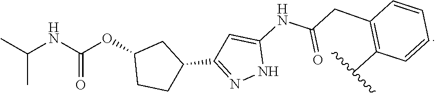 [SMILES: *c1ccccc1CC(=O)Nc1cc([C@@H]2CC[C@H](OC(=O)NC(C)C)C2)n[nH]1]

In some embodiments, CBM is

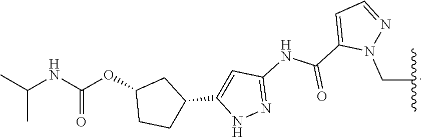 [SMILES: *Cn1nccc1C(=O)Nc1cc([C@@H]2CC[C@H](OC(=O)NC(C)C)C2)[nH]n1]

In some embodiments, CBM is

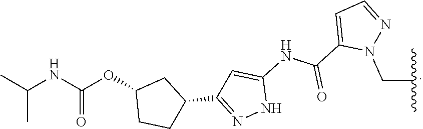 [SMILES: *Cn1nccc1C(=O)Nc1cc([C@@H]2CC[C@H](OC(=O)NC(C)C)C2)n[nH]1]

In some embodiments, CBM is

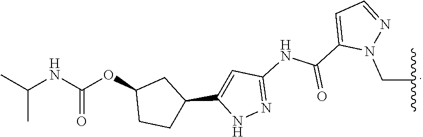 [SMILES: *Cn1nccc1C(=O)Nc1cc([C@H]2CC[C@@H](OC(=O)NC(C)C)C2)[nH]n1]

In some embodiments, CBM is

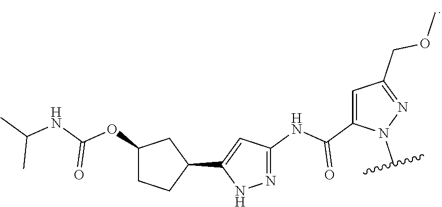 [SMILES: *n1nc(COC)cc1C(=O)Nc1cc([C@H]2CC[C@@H](OC(=O)NC(C)C)C2)[nH]n1]

In some embodiments, CBM is

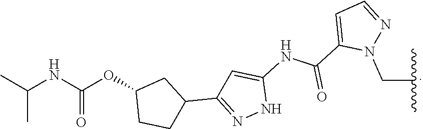 [SMILES: *Cn1nccc1C(=O)Nc1cc(C2CC[C@H](OC(=O)NC(C)C)C2)n[nH]1]

In some embodiments, CBM is

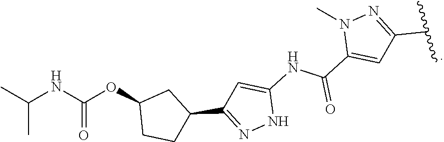 [SMILES: *c1cc(C(=O)Nc2cc([C@H]3CC[C@@H](OC(=O)NC(C)C)C3)n[nH]2)n(C)n1]

In some embodiments, CBM is

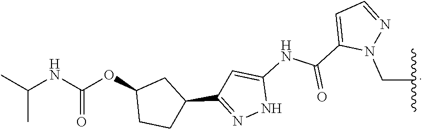 [SMILES: *Cn1nccc1C(=O)Nc1cc([C@H]2CC[C@@H](OC(=O)NC(C)C)C2)n[nH]1]

In some embodiments, CBM is

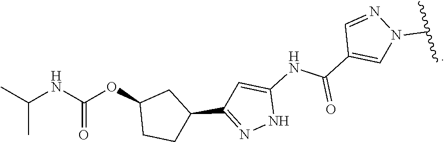 [SMILES: *n1cc(C(=O)Nc2cc([C@H]3CC[C@@H](OC(=O)NC(C)C)C3)n[nH]2)cn1]

In some embodiments, CBM is

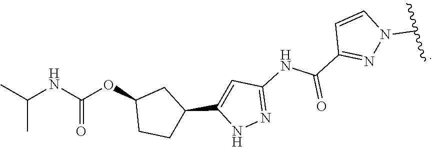 [SMILES: *n1ccc(C(=O)Nc2cc([C@H]3CC[C@@H](OC(=O)NC(C)C)C3)[nH]n2)n1]

In some embodiments, CBM is

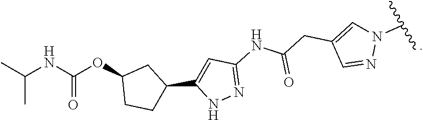 [SMILES: *n1cc(CC(=O)Nc2cc([C@H]3CC[C@@H](OC(=O)NC(C)C)C3)[nH]n2)cn1]

In some embodiments, CBM is

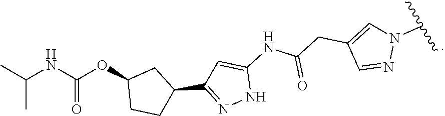 [SMILES: *n1cc(CC(=O)Nc2cc([C@H]3CC[C@@H](OC(=O)NC(C)C)C3)n[nH]2)cn1]

In some embodiments, CBM is

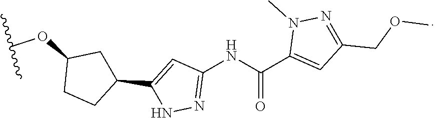 [SMILES: *O[C@@H]1CC[C@H](c2cc(NC(=O)c3cc(COC)nn3C)n[nH]2)C1]

In some embodiments, CBM is

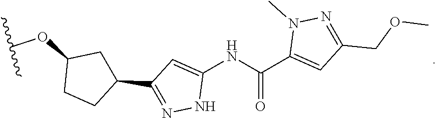 [SMILES: *O[C@@H]1CC[C@H](c2cc(NC(=O)c3cc(COC)nn3C)[nH]n2)C1]

In some embodiments, CBM is

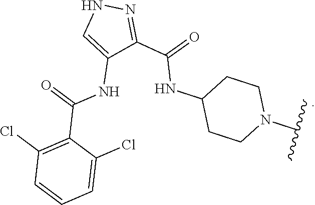 [SMILES: *N1CCC(NC(=O)c2n[nH]cc2NC(=O)c2c(Cl)cccc2Cl)CC1]

In some embodiments, CBM is selected from those depicted in Table 1, below.

In certain embodiments, the present invention provides a compound of formula I, wherein CBM is a CDK2 binding moiety thereby forming a compound of formula I-c-1:

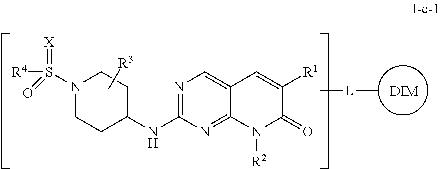

or a pharmaceutically acceptable salt thereof, wherein L and DIM are as defined above and described in embodiments herein, and wherein each of the variables, R¹, R², R³ and R⁴ are as defined and described in WO 2021/254384, the entirety of which is herein incorporated by reference.

In certain embodiments, the present invention provides a compound of formula I, wherein CBM is a CDK2 binding moiety thereby forming a compound of formula I-c-2:

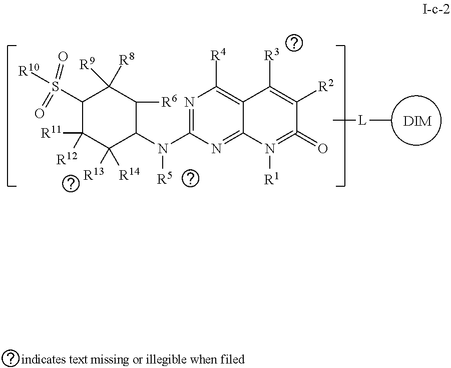

or a pharmaceutically acceptable salt thereof, wherein L and DIM are as defined above and described in embodiments herein, and wherein each of the variables R¹, R², R³, R⁴, R⁵, R⁶, R⁷, R⁸, R⁹, R¹⁰, R¹¹, R¹², R¹³, R¹⁴, and R¹⁵ are as defined and described in WO 2021/249258, the entirety of which is herein incorporated by reference.

In certain embodiments, the present invention provides a compound of formula I, wherein CBM is a CDK2 binding moiety thereby forming a compound of formula I-c-3:

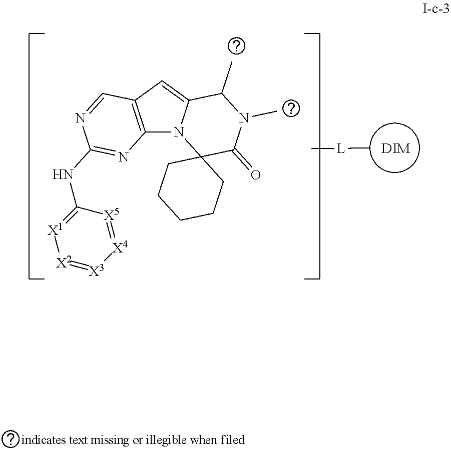

or a pharmaceutically acceptable salt thereof, wherein L and DIM are as defined above and described in embodiments herein, and wherein each of the variables X¹, X², X³, X⁴, X⁵, R³ and R are as defined and described in WO 2021/236650, the entirety of which is herein incorporated by reference.

In certain embodiments, the present invention provides a compound of formula I, wherein CBM is a CDK2 binding moiety thereby forming a compound of formula I-c-4 and I-c-5:

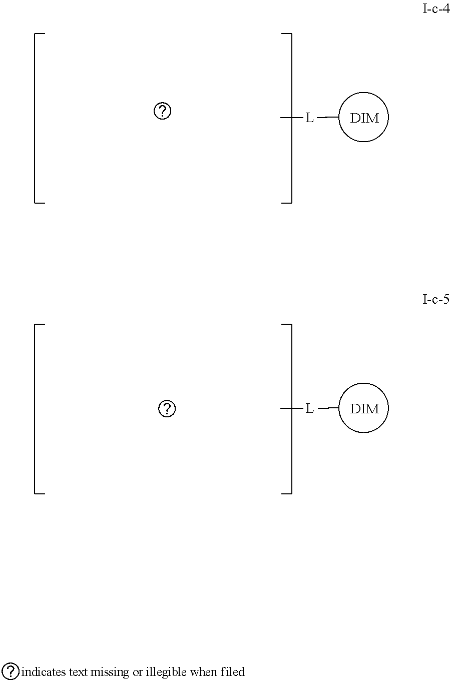

or a pharmaceutically acceptable salt thereof, wherein L and DIM are as defined above and described in embodiments herein, and wherein each of the variables R¹ and R² are as defined and described in CN11289271, the entirety of which is herein incorporated by reference.

In certain embodiments, the present invention provides a compound of formula I, wherein CBM is a CDK2 binding moiety thereby forming a compound of formula I-c-6:

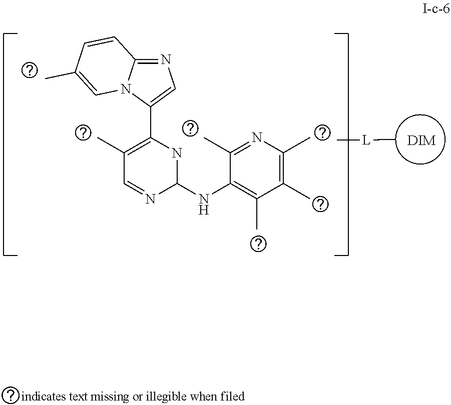

or a pharmaceutically acceptable salt thereof, wherein L and DIM are as defined above and described in embodiments herein, and wherein each of the variables R¹, R², R³, R⁴, R⁵ and R⁶ are as defined and described in WO 2021/072475, the entirety of which is herein incorporated by reference.

In certain embodiments, the present invention provides a compound of formula I, wherein CBM is a CDK2 binding moiety thereby forming a compound of formula I-c-1:

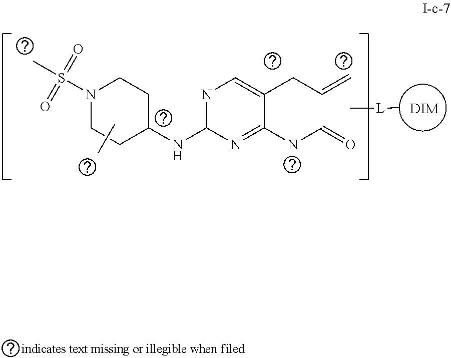

or a pharmaceutically acceptable salt thereof, wherein L and DIM are as defined above and described in embodiments herein, and wherein each of the variables R¹, R², R³ and R⁴ are as defined and described in WO 2021/254384, the entirety of which is herein incorporated by reference.

In certain embodiments, the present invention provides a compound of formula I, wherein CBM is a CDK2 binding moiety thereby forming a compound of formula I-c-8:

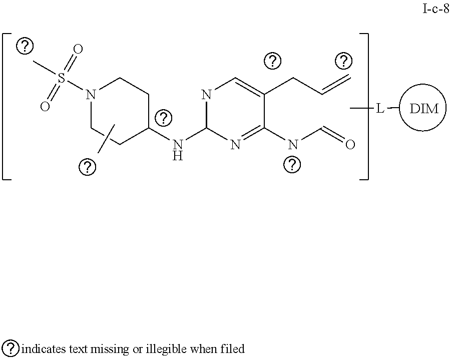

or a pharmaceutically acceptable salt thereof, wherein L and DIM are as defined above and described in embodiments herein, and wherein each of the variables R¹, R², R³, R⁴, R⁵, R⁶, R⁷ and n are as defined and described in WO 2021/073593, the entirety of which is herein incorporated by reference.

In certain embodiments, the present invention provides a compound of formula I, wherein CBM is a CDK2 binding moiety thereby forming a compound of formula I-c-9:

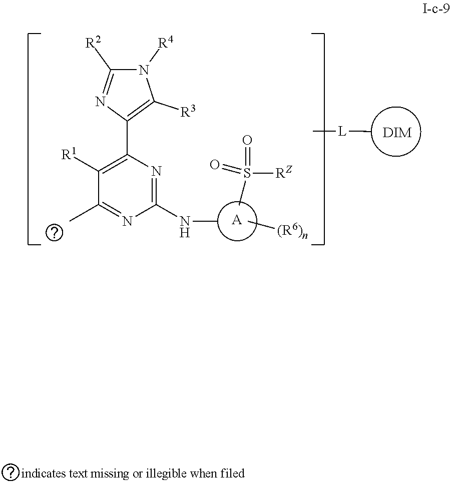

or a pharmaceutically acceptable salt thereof, wherein L and DIM are as defined above and described in embodiments herein, and wherein each of the variables R^{z}, A, R¹, R², R³, R⁴, R⁶, R⁷ and are as defined and described in WO 2021/030537, the entirety of which is herein incorporated by reference.

In certain embodiments, the present invention provides a compound of formula I, wherein CBM is a CDK2 binding moiety thereby forming a compound of formula I-c-10, I-c-11, and I-c-12:

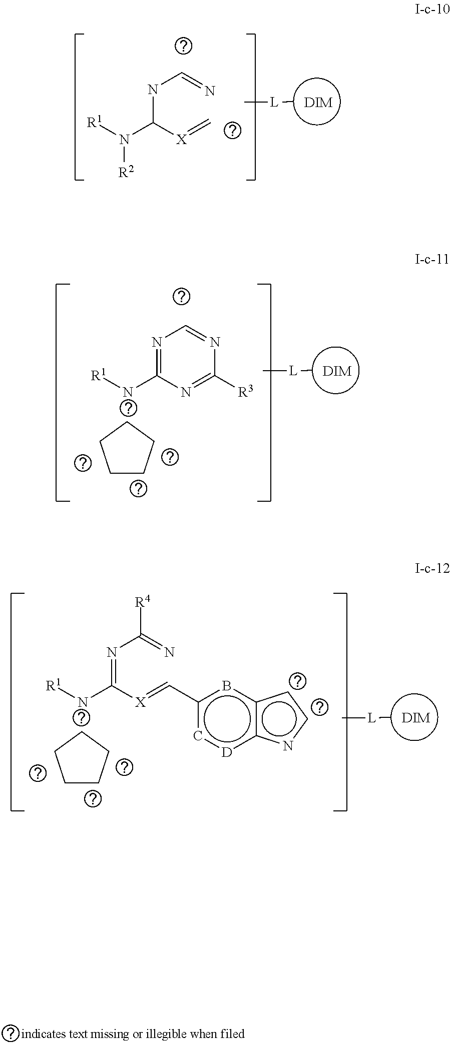

or a pharmaceutically acceptable salt thereof, wherein L and DIM are as defined above and described in embodiments herein, and wherein each of the variables X, Y, B, D, C, D, E, F, s, t, n, R¹, R², R³, R⁴ and R are as defined and described in CN 113698391, the entirety of which is herein incorporated by reference.

In certain embodiments, the present invention provides a compound of formula I, wherein CBM is a CDK2 binding moiety thereby forming a compound of formula I-c-13:

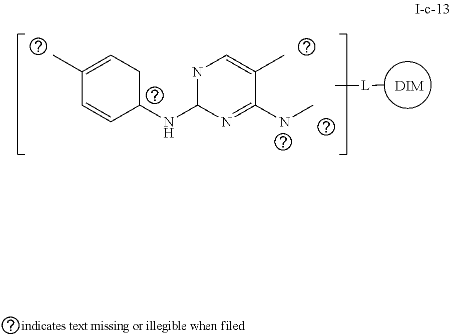

or a pharmaceutically acceptable salt thereof, wherein L and DIM are as defined above and described in embodiments herein, and wherein each of the variables R¹, R², and R are as defined and described in CN113999210, the entirety of which is herein incorporated by reference.

In certain embodiments, the present invention provides a compound of formula I, wherein CBM is a CDK2 binding moiety thereby forming a compound of formula I-c-14:

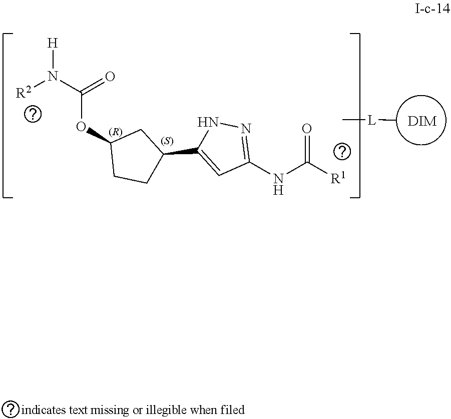

or a pharmaceutically acceptable salt thereof, wherein L and DIM are as defined above and described in embodiments herein, and wherein each of the variables R¹, and R² are as defined and described in WO 2022/018596, the entirety of which is herein incorporated by reference.

In certain embodiments, the present invention provides a compound of formula I, wherein CBM is a CDK2 binding moiety thereby forming a compound of formula I-c-15:

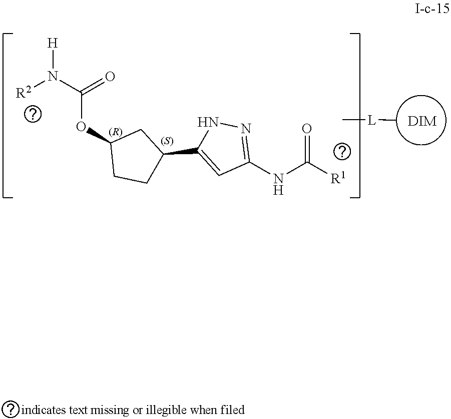

or a pharmaceutically acceptable salt thereof, wherein L and DIM are as defined above and described in embodiments herein, and wherein each of the variables R¹ and R² are as defined and described in WO 2022/018667, the entirety of which is herein incorporated by reference.

In certain embodiments, the present invention provides a compound of formula I, wherein CBM is a CDK2 binding moiety thereby forming a compound of formula I-c-16:

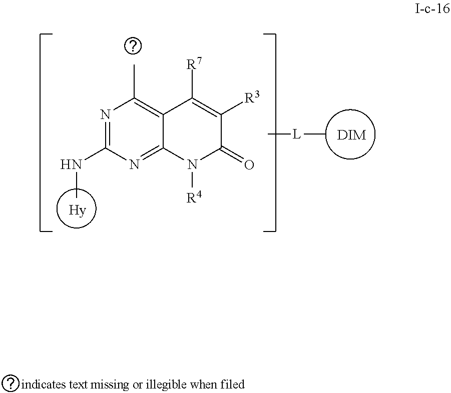

or a pharmaceutically acceptable salt thereof, wherein L and DIM are as defined above and described in embodiments herein, and wherein each of the variables R¹, R², R³, R⁴ and Hy are as defined and described in WO 2022/015670, the entirety of which is herein incorporated by reference.

In certain embodiments, the present invention provides a compound of formula I, wherein CBM is a CDK2 binding moiety thereby forming a compound of formula I-c-17:

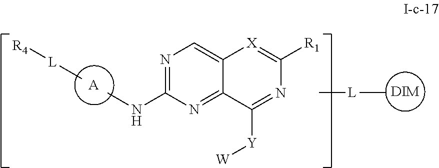

or a pharmaceutically acceptable salt thereof, wherein L and DIM are as defined above and described in embodiments herein, and wherein each of the variables X, Y, W, A, L, R¹ and R⁴ are as defined and described in WO 2022/037592, the entirety of which is herein incorporated by reference.

In certain embodiments, the present invention provides a compound of formula I, wherein CBM is a CDK2 binding moiety thereby forming a compound of formula I-c-18:

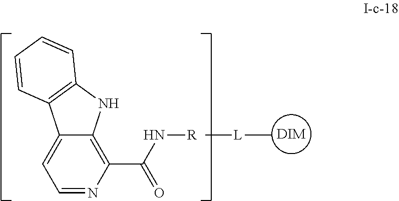

or a pharmaceutically acceptable salt thereof, wherein L and DIM are as defined above and described in embodiments herein, and wherein each of the variables Rare as defined and described in CN114380822, the entirety of which is herein incorporated by reference.

In certain embodiments, the present invention provides a compound of formula I, wherein CBM is a CDK2 binding moiety thereby forming a compound of formula I-c-19:

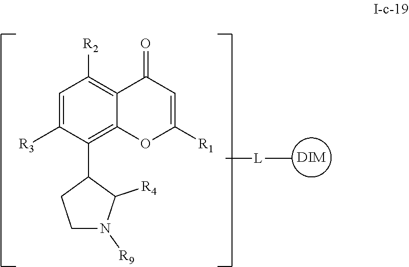

or a pharmaceutically acceptable salt thereof, wherein L and DIM are as defined above and described in embodiments herein, and wherein each of the variables R¹, R², R³, R⁴ and R⁹ are as defined and described in WO 2022/109307, the entirety of which is herein incorporated by reference.

In certain embodiments, the present invention provides a compound of formula I, wherein CBM is a CDK2 binding moiety thereby forming a compound of formula I-c-20:

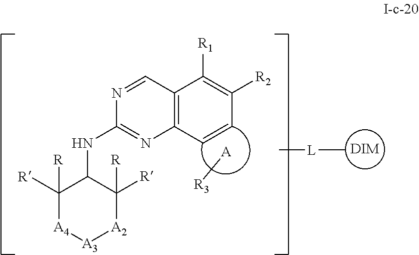

or a pharmaceutically acceptable salt thereof, wherein L and DIM are as defined above and described in embodiments herein, and wherein each of the variables A, A², A³, A⁴, R¹, R², Rand R′ are as defined and described in WO 2022/111634, the entirety of which is herein incorporated by reference.

In certain embodiments, the present invention provides a compound of formula I, wherein CBM is a CDK2 binding moiety thereby forming a compound of formula I-c-21:

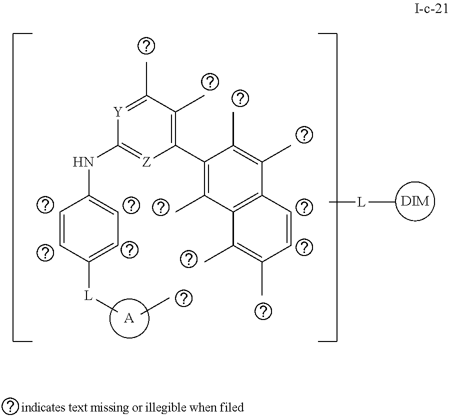

or a pharmaceutically acceptable salt thereof, wherein L and DIM are as defined above and described in embodiments herein, and wherein each of the variables A, L, Y, Z, Y¹, Y², X¹, X², X³, X⁴, R^{c}, R^{d}, R^{e}, R^{f}, R^{g}, R⁵, R⁶, R⁷ and n are as defined and described in WO 2022/113003, the entirety of which is herein incorporated by reference.

In certain embodiments, the present invention provides a compound of formula I, wherein CBM is a CDK2 binding moiety thereby forming a compound of formula I-c-22:

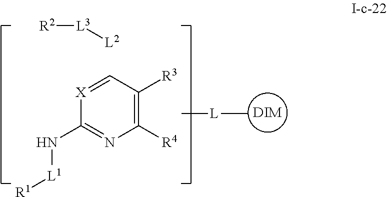

or a pharmaceutically acceptable salt thereof, wherein L and DIM are as defined above and described in embodiments herein, and wherein each of the variables L¹, L², L³, X, R¹, R², R³ and R⁴ are as defined and described in WO 2022/113621, the entirety of which is herein incorporated by reference.

In certain embodiments, the present invention provides a compound of formula I, wherein CBM is a CDK2 binding moiety thereby forming a compound of formula I-c-23:

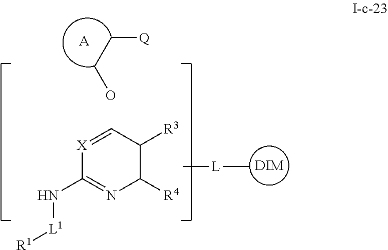

or a pharmaceutically acceptable salt thereof, wherein L and DIM are as defined above and described in embodiments herein, and wherein each of the variables L¹, X, A, Q, R¹, R³ and R⁴ are as defined and described in WO 2022/113621, the entirety of which is herein incorporated by reference.

In certain embodiments, the present invention provides a compound of formula I, wherein CBM is a CDK2 binding moiety thereby forming a compound of formula I-c-24:

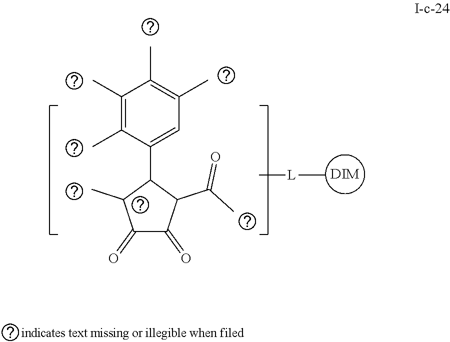

or a pharmaceutically acceptable salt thereof, wherein L and DIM are as defined above and described in embodiments herein, and wherein each of the variables R², R³, R¹¹, R¹², R¹³, and R⁴ are as defined and described in CN114591213, the entirety of which is herein incorporated by reference.

In certain embodiments, the present invention provides a compound of formula I, wherein CBM is a CDK2 binding moiety thereby forming a compound of formula I-c-25:

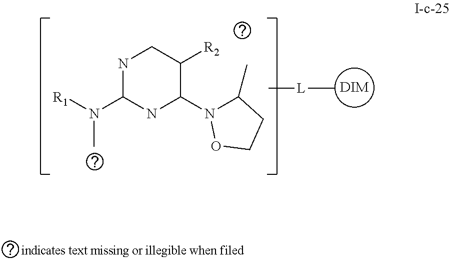

or a pharmaceutically acceptable salt thereof, wherein L and DIM are as defined above and described in embodiments herein, and wherein each of the variables R¹, R², A¹ and A² are as defined and described in WO 2022/131741, the entirety of which is herein incorporated by reference.

In certain embodiments, the present invention provides a compound of formula I, wherein CBM is a CDK2 binding moiety thereby forming a compound of formula I-c-26:

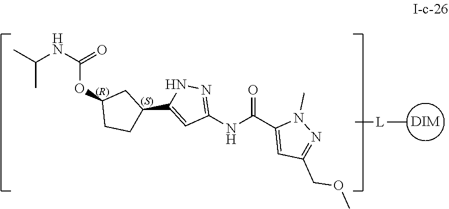

or a pharmaceutically acceptable salt thereof, wherein L and DIM are as defined above and described in embodiments herein, and in WO 2022/137106, the entirety of which is herein incorporated by reference.

In certain embodiments, the present invention provides a compound of formula I, wherein CBM is a CDK2 binding moiety thereby forming a compound of formula I-c-27:

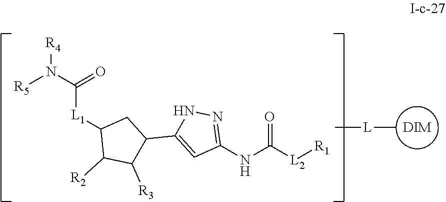

or a pharmaceutically acceptable salt thereof, wherein L and DIM are as defined above and described in embodiments herein, and wherein each of the variables R¹, R², R³, R⁴ and R are as defined and described in WO 2022/135442, the entirety of which is herein incorporated by reference.

In certain embodiments, the present invention provides a compound of formula I, wherein CBM is a CDK2 binding moiety thereby forming a compound of formula I-c-28 and I-c-29:

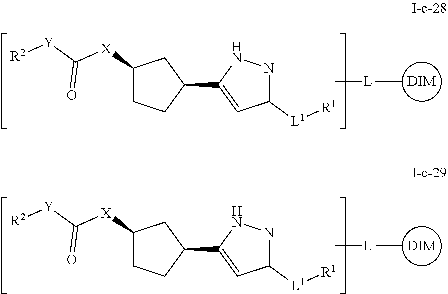

or a pharmaceutically acceptable salt thereof, wherein L and DIM are as defined above and described in embodiments herein, and wherein each of the variables R¹, R², R and R⁴ are as defined and described in WO 2022/135365, the entirety of which is herein incorporated by reference.

In certain embodiments, the present invention provides a compound of formula I, wherein CBM is a CDK2 binding moiety thereby forming a compound of formula I-c-32:

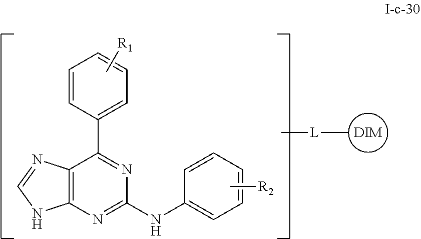

or a pharmaceutically acceptable salt thereof, wherein L and DIM are as defined above and described in embodiments herein, and wherein each of the variables R¹, and R² are as defined and described in CN114685507, the entirety of which is herein incorporated by reference.

In certain embodiments, the present invention provides a compound of formula I, wherein CBM is a CDK2 binding moiety thereby forming a compound of formula I-c-31:

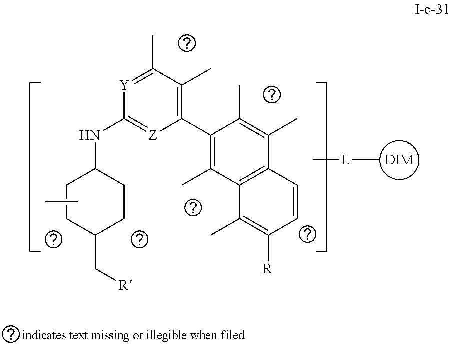

or a pharmaceutically acceptable salt thereof, wherein L and DIM are as defined above and described in embodiments herein, and wherein each of the variables Y, Z, Y¹, Y², R^{c}, R^{d}, R^{e}, R^{f}, R^{g}, R¹, R², R³, m and n are as defined and described in WO 2022/149057, the entirety of which is herein incorporated by reference.

In certain embodiments, the present invention provides a compound of formula I, wherein CBM is a CDK2 binding moiety thereby forming a compound of formula I-c-32:

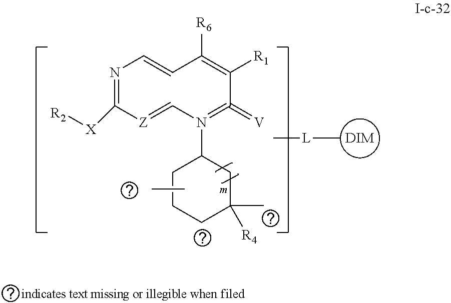

or a pharmaceutically acceptable salt thereof, wherein L and DIM are as defined above and described in embodiments herein, and wherein each of the variables X, Y, Z, R¹, R², R³, R⁴, R⁵, R⁶, m, n and p are as defined and described in WO 2022/152259, the entirety of which is herein incorporated by reference.

In certain embodiments, the present invention provides a compound of formula I, wherein CBM is a CDK2 binding moiety thereby forming a compound of formula I-c-33:

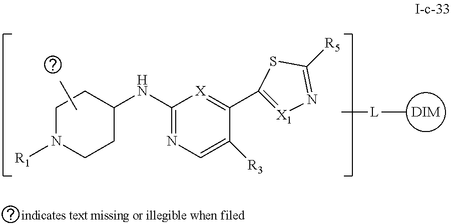

or a pharmaceutically acceptable salt thereof, wherein L and DIM are as defined above and described in embodiments herein, and wherein each of the variables m, X, X₁, R¹, R², R³ and R⁵ are as defined and described in WO 2022/155941, the entirety of which is herein incorporated by reference.

In certain embodiments, the present invention provides a compound of formula I, wherein CBM is a CDK2 binding moiety thereby forming a compound of formula I-c-34:

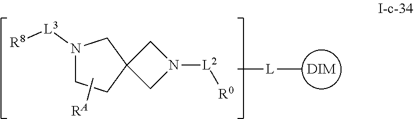

or a pharmaceutically acceptable salt thereof, wherein L and DIM are as defined above and described in embodiments herein, and wherein each of the variables L³, L³, R^{A}, R⁶ and R⁸ are as defined and described in WO 2022/165513, the entirety of which is herein incorporated by reference.

In certain embodiments, the present invention provides a compound of formula I, wherein CBM is a CDK2 binding moiety thereby forming a compound of formula I-c-35:

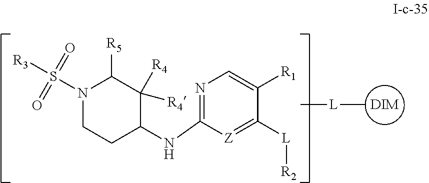

or a pharmaceutically acceptable salt thereof, wherein L and DIM are as defined above and described in embodiments herein, and wherein each of the variables R₁, R₂, R₃, R₄, R₄, and R₅ are as defined and described in WO 2022/166793, the entirety of which is herein incorporated by reference.

In certain embodiments, the present invention provides a compound of formula I, wherein CBM is a CDK2 binding moiety thereby forming a compound of formula I-c-36:

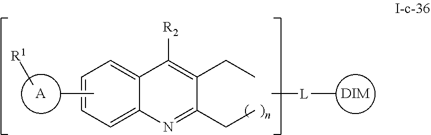

or a pharmaceutically acceptable salt thereof, wherein L and DIM are as defined above and described in embodiments herein, and wherein each of the variables A, R¹, R² and n are as defined and described in CN114853672, the entirety of which is herein incorporated by reference.

In certain embodiments, the present invention provides a compound of formula I, wherein CBM is a CDK2 binding moiety thereby forming a compound of formula I-c-37 and I-c-38:

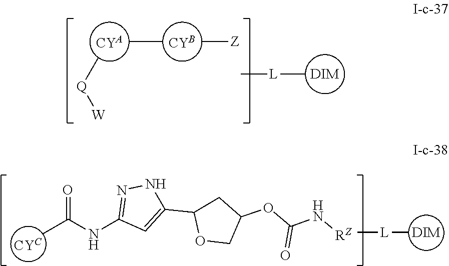

or a pharmaceutically acceptable salt thereof, wherein L and DIM are as defined above and described in embodiments herein, and wherein each of the variables Cy^{A} Cy^{B}, Cy^{C}, Z, and R^{z} are as defined and described in WO 2022/174031, the entirety of which is herein incorporated by reference.

In certain embodiments, the present invention provides a compound of formula I, wherein CBM is a CDK2 binding moiety thereby forming a compound of formula I-c-39:

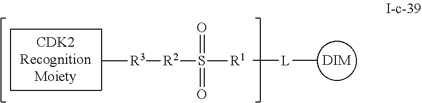

or a pharmaceutically acceptable salt thereof, wherein L and DIM are as defined above and described in embodiments herein, and wherein each of the variables R¹, R², R³ and CDK2 Recognition Moiety are as defined and described in WO 2022/187693, the entirety of which is herein incorporated by reference.

In certain embodiments, the present invention provides a compound of formula I, wherein CBM is a CDK2 binding moiety thereby forming a compound of formula I-c-40:

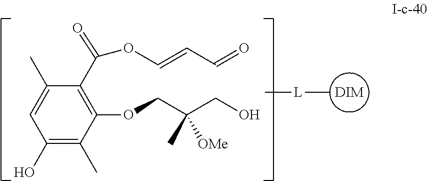

or a pharmaceutically acceptable salt thereof, wherein L and DIM are as defined above and described in embodiments herein, and as described in WO 2022/187611, the entirety of which is herein incorporated by reference.

In certain embodiments, the present invention provides a compound of formula I, wherein CBM is a CDK2 binding moiety thereby forming a compound of formula I-c-41 and I-c-42:

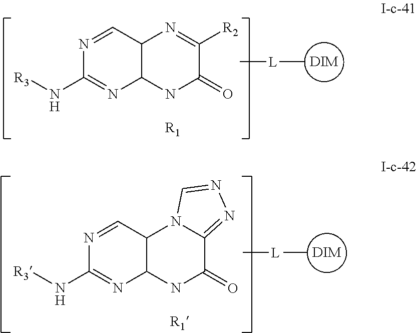

or a pharmaceutically acceptable salt thereof, wherein L and DIM are as defined above and described in embodiments herein, and wherein each of the variables R¹, R², and R are as defined and described in CN115010711, the entirety of which is herein incorporated by reference.

In certain embodiments, the present invention provides a compound of formula I, wherein CBM is a CDK2 binding moiety thereby forming a compound of formula I-c-43:

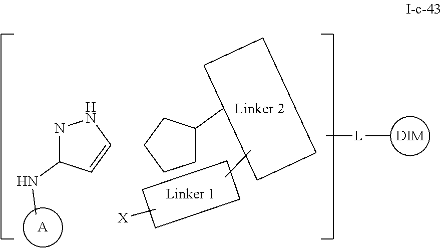

or a pharmaceutically acceptable salt thereof, wherein L and DIM are as defined above and described in embodiments herein, and wherein each of the variables A, Linker 1 and Linker 2 are as defined and described in WO 2022/206888, the entirety of which is herein incorporated by reference.

In certain embodiments, the present invention provides a compound of formula I, wherein CBM is a CDK2 binding moiety thereby forming a compound of formula I-c-44:

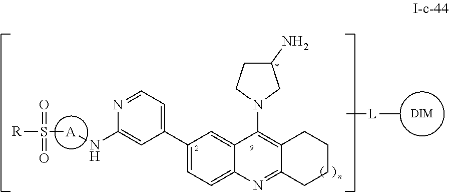

or a pharmaceutically acceptable salt thereof, wherein L and DIM are as defined above and described in embodiments herein, and wherein each of the variables A, R and n are as defined and described in CN 115160298, the entirety of which is herein incorporated by reference.

In certain embodiments, the present invention provides a compound of formula I, wherein CBM is a CDK2 binding moiety thereby forming a compound of formula I-c-45:

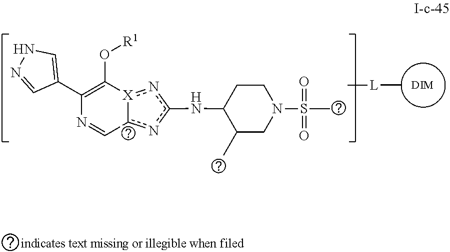

or a pharmaceutically acceptable salt thereof, wherein L and DIM are as defined above and described in embodiments herein, and wherein each of the variables X, Y, and R¹ are as defined and described in US 2022/0340579, the entirety of which is herein incorporated by reference.

In certain embodiments, the present invention provides a compound of formula I, wherein CBM is a CDK2 binding moiety thereby forming a compound of formula I-c-46:

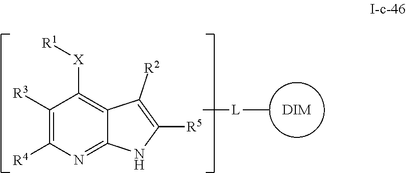

or a pharmaceutically acceptable salt thereof, wherein L and DIM are as defined above and described in embodiments herein, and wherein each of the variables R₁, R₂, R₃, R₄, R₅, and X are as defined and described in WO 2022/245776, the entirety of which is herein incorporated by reference.

In certain embodiments, the present invention provides a compound of formula I, wherein CBM is a CDK2 binding moiety thereby forming a compound of formula I-c-47:

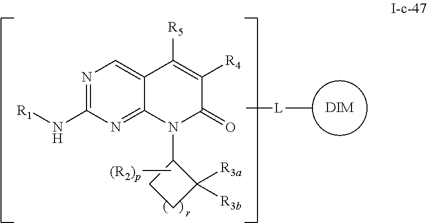

or a pharmaceutically acceptable salt thereof, wherein L and DIM are as defined above and described in embodiments herein, and wherein each of the variables R₁, R₂, R₃ₐ, R_{3b}, R₄, R₅, p, and r are as defined and described in WO 2022/258023, the entirety of which is herein incorporated by reference.

In certain embodiments, the present invention provides a compound of formula I, wherein CBM is a CDK2 binding moiety thereby forming a compound of formula I-c-48:

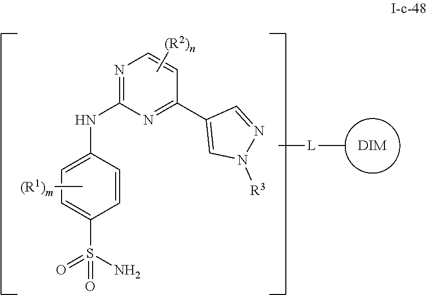

or a pharmaceutically acceptable salt thereof, wherein L and DIM are as defined above and described in embodiments herein, and wherein each of the variables R₁, R₂, R₃, m, and n are as defined and described in WO 2022/266190, the entirety of which is herein incorporated by reference.

In certain embodiments, the present invention provides a compound of formula I, wherein CBM is a CDK2 binding moiety thereby forming a compound of formula I-c-49:

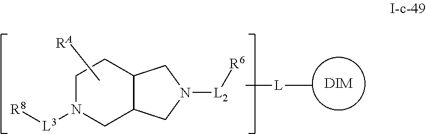

or a pharmaceutically acceptable salt thereof, wherein L and DIM are as defined above and described in embodiments herein, and wherein each of the variables R⁸, R^{A}, L³, L₂, and R are as defined and described in WO 2022/272106, the entirety of which is herein incorporated by reference.

In certain embodiments, the present invention provides a compound of formula I, wherein CBM is a CDK2 binding moiety thereby forming a compound of formula I-c-50:

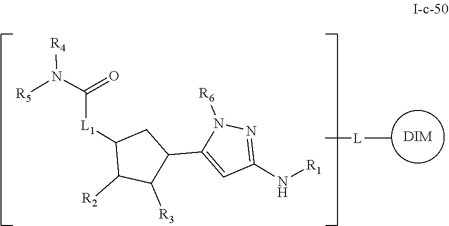

or a pharmaceutically acceptable salt thereof, wherein L and DIM are as defined above and described in embodiments herein, and wherein each of the variables R₁, R₂, R₃, R₄, R₅, R₆, and L₁ are as defined and described in WO 2023/274397, the entirety of which is herein incorporated by reference.

In certain embodiments, the present invention provides a compound of formula I, wherein CBM is a CDK2 binding moiety thereby forming a compound of formula I-c-51:

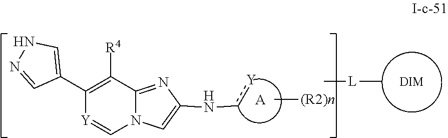

or a pharmaceutically acceptable salt thereof, wherein L and DIM are as defined above and described in embodiments herein, and wherein each of the variables A, Y, R₂, R₄, and n are as defined and described in US 2023/002376, the entirety of which is herein incorporated by reference.

In certain embodiments, the present invention provides a compound of formula I, wherein CBM is a CDK2 binding moiety thereby forming a compound of formula I-c-52:

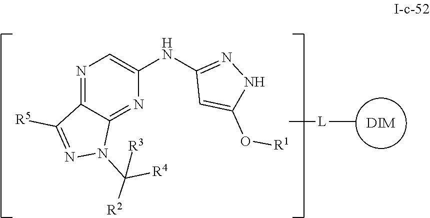

or a pharmaceutically acceptable salt thereof, wherein L and DIM are as defined above and described in embodiments herein, and wherein each of the variables R¹, R², R³, R⁴, and Rare as defined and described in WO 2023/278326, the entirety of which is herein incorporated by reference.

In certain embodiments, the present invention provides a compound of formula I, wherein CBM is a CDK2 binding moiety thereby forming a compound of formula I-c-53:

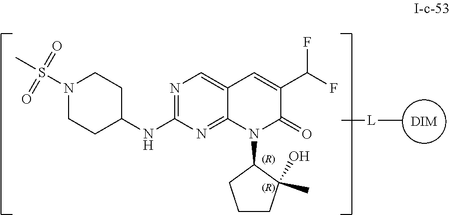

or a pharmaceutically acceptable salt thereof, wherein L and DIM are as defined above and described in embodiments herein, and described in WO 2023/281413, the entirety of which is herein incorporated by reference.

In certain embodiments, the present invention provides a compound of formula I, wherein CBM is a CDK2 binding moiety thereby forming a compound of formula I-c-54:

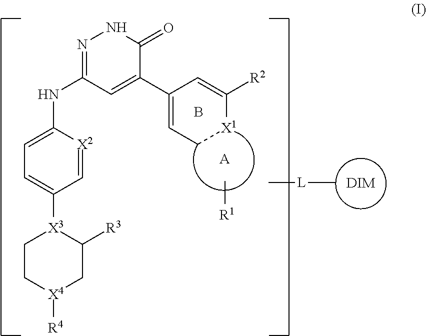

or a pharmaceutically acceptable salt thereof, wherein L and DIM are as defined above and described in embodiments herein, and wherein each of the variables R¹, R², R³, R⁴, X¹, X², X³, A and B are as defined and described in CN 115650968, the entirety of which is herein incorporated by reference.

In certain embodiments, the present invention provides a compound of formula I, wherein CBM is a CDK2 binding moiety thereby forming a compound of formula I-c-55:

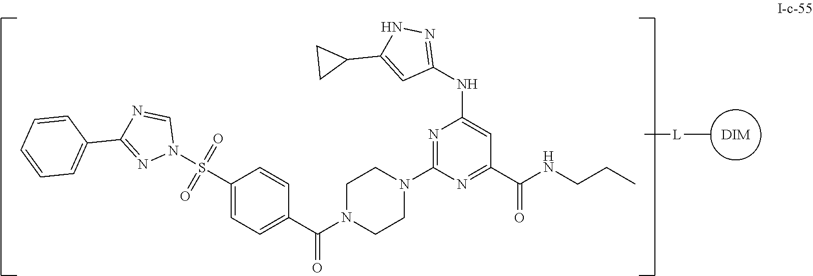

or a pharmaceutically acceptable salt thereof, wherein L and DIM are as defined above and described in embodiments herein, and described in WO 2023/023376 and WO 2023/023664, the entireties of which are herein incorporated by reference.

In certain embodiments, the present invention provides a compound of formula I, wherein CBM is a CDK2 binding moiety thereby forming a compound of formula I-c-56:

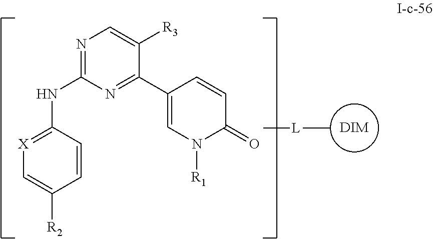

or a pharmaceutically acceptable salt thereof, wherein L and DIM are as defined above and described in embodiments herein, and wherein each of the variables R₁, R₂, R₃, and X are as defined and described in CN 115703760, the entirety of which is herein incorporated by reference.

In certain embodiments, the present invention provides a compound of formula I, wherein CBM is a CDK2 binding moiety thereby forming a compound of formula I-c-57:

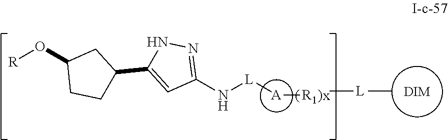

or a pharmaceutically acceptable salt thereof, wherein L and DIM are as defined above and described in embodiments herein, and wherein each of the variables R₁, R, A, L, and x are as defined and described in CN 115806551, the entirety of which is herein incorporated by reference.

In certain embodiments, the present invention provides a compound of formula I, wherein CBM is a CDK2 binding moiety thereby forming a compound of formula I-c-58:

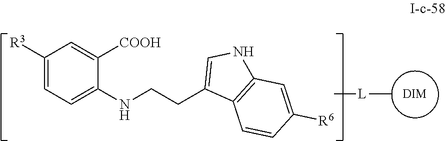

or a pharmaceutically acceptable salt thereof, wherein L and DIM are as defined above and described in embodiments herein, and wherein each of the variables R³ and R⁶, are as defined and described in Faber et al., J. Med. Chem. 2023, 66, 3, 1928-1940, the entirety of which is herein incorporated by reference.

In certain embodiments, the present invention provides a compound of formula I, wherein CBM is a CDK2 binding moiety thereby forming a compound of formula I-c-59:

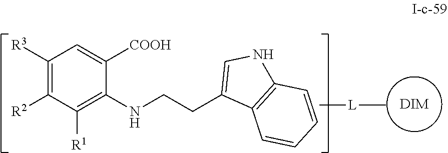

or a pharmaceutically acceptable salt thereof, wherein L and DIM are as defined above and described in embodiments herein, and wherein each of the variables R¹, R², and R, are as defined and described in Faber et al., J. Med. Chem. 2023, 66, 3, 1928-1940, the entirety of which is herein incorporated by reference.

In certain embodiments, the present invention provides a compound of formula I, wherein CBM is a CDK2 binding moiety thereby forming a compound of formula I-c-60:

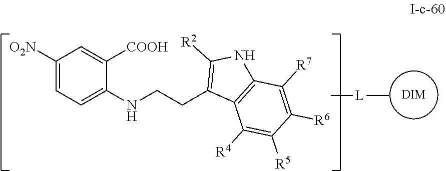

or a pharmaceutically acceptable salt thereof, wherein L and DIM are as defined above and described in embodiments herein, and wherein each of the variables R², R⁴, R⁵, R⁶, and R⁷ are as defined and described in Faber et al., J. Med. Chem. 2023, 66, 3, 1928-1940, the entirety of which is herein incorporated by reference.

In certain embodiments, the present invention provides a compound of formula I, wherein CBM is a CDK2 binding moiety thereby forming a compound of formula I-c-61:

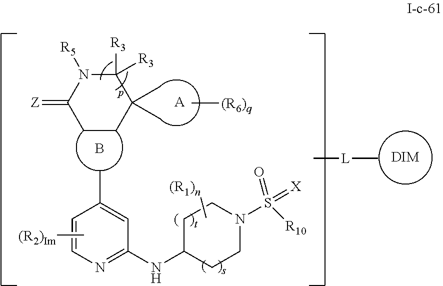

or a pharmaceutically acceptable salt thereof, wherein L and DIM are as defined above and described in embodiments herein, and wherein each of the variables R¹, R², R³, R⁵, R⁶, R¹⁰, Ring A, Ring B, X, Z, p, q, m, n, and s are as defined and described in WO2023/150612, the entirety of which is herein incorporated by reference.

In certain embodiments, the present invention provides a compound of formula I, wherein CBM is a CDK2 binding moiety thereby forming a compound of formula I-c-62:

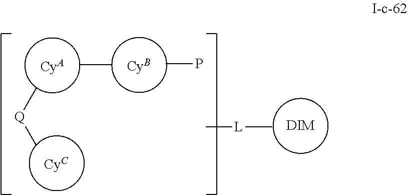

or a pharmaceutically acceptable salt thereof, wherein L and DIM are as defined above and described in embodiments herein, and wherein each of the variables Cy^{A}, Cy^{B}, Cy^{C}, Q and P are as defined and described in WO2023/154426, the entirety of which is herein incorporated by reference.

Ligase Binding Moiety (LBM)

In some embodiments, DIM is LBM. In some embodiments, LBM is an E3 ligase ligand well known to one of ordinary skill in the art including those described in M. Toure, C. M. Crews, Angew. Chem. Int. Ed. 2016, 55, 1966, T. Uehara et al. Nature Chemical Biology 2017, 13, 675, WO 2017/176708, US 2017/0281784, WO 2017/161119, WO 2017/176957, WO 2017/176958, WO 2015/160845, US 2015/0291562, WO 2016/197032, WO 2016/105518, US 2018/0009779, WO 2017/007612, 2018/0134684, WO 2013/106643, US 2014/0356322, WO 2002/020740, US 2002/0068063, WO 2012/078559, US 2014/0302523, WO 2012/003281, US 2013/0190340, US 2016/0022642, WO 2014/063061, US 2015/0274738, WO 2016/118666, US 2016/0214972, WO 2016/149668, US 2016/0272639, WO 2016/169989, US 2018/0118733, WO 2016/197114, US 2018/0147202, WO 2017/011371, US 2017/0008904, WO 2017/011590, US 2017/0037004, WO 2017/079267, US 2017/0121321, WO 2017/117473, WO 2017/117474, WO 2013/106646, WO 2014/108452, WO 2017/197036, US 2019/0076540, WO 2017/197046, US 2019/0076542, WO 2017/197051, US 2019/0076539, WO 2017/197055, US 2019/0076541, and WO 2017/197056, the entirety of each of which is herein incorporated by reference.

As defined herein and described below, wherein a formula is depicted using square brackets, e.g.,

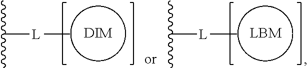

L is attached to a modifiable carbon, oxygen, or nitrogen atom within DIM or LBM including substitution or replacement of a defined group in DIM or LBM.

In certain embodiments, the present invention provides a compound of formula I, wherein DIM (e.g., LBM) is a cereblon E3 ubiquitin ligase binding moiety thereby forming a compound of formula I-aa:

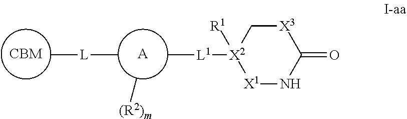

or a pharmaceutically acceptable salt thereof, wherein L and CBM are as defined above and described herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

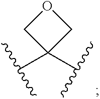 [SMILES: *C1(*)COC1]

X² is a carbon atom or silicon atom;
X³ is a bivalent moiety selected from —CR₂—, —NR—, —O—, —S—, or —Si(R²)—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, deuterium, —R, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a bi- or tricyclic ring selected from

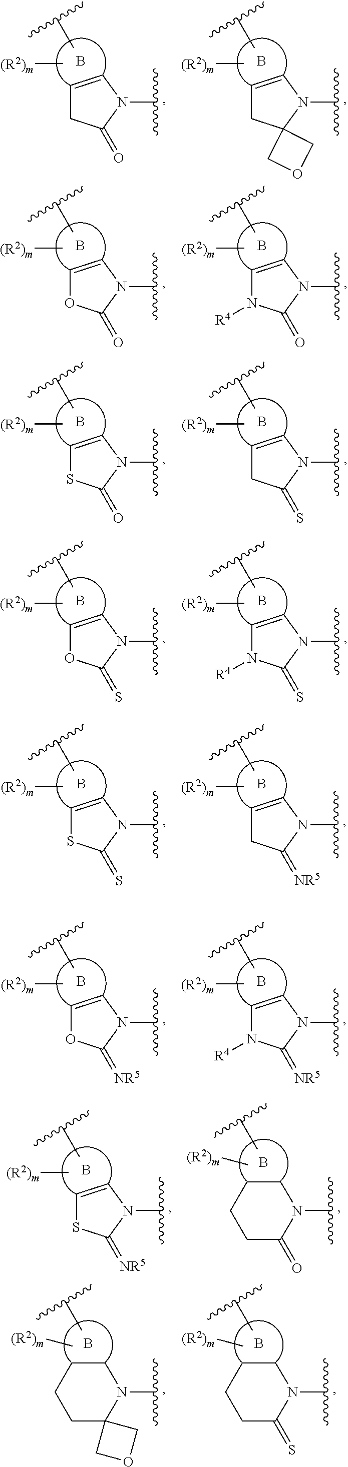

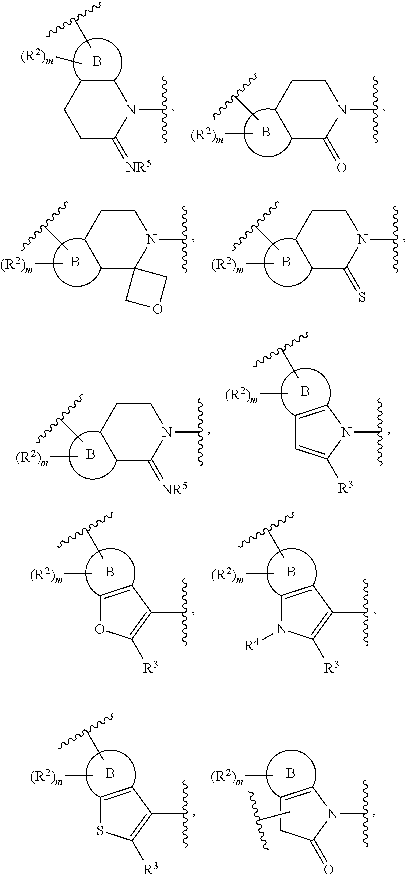

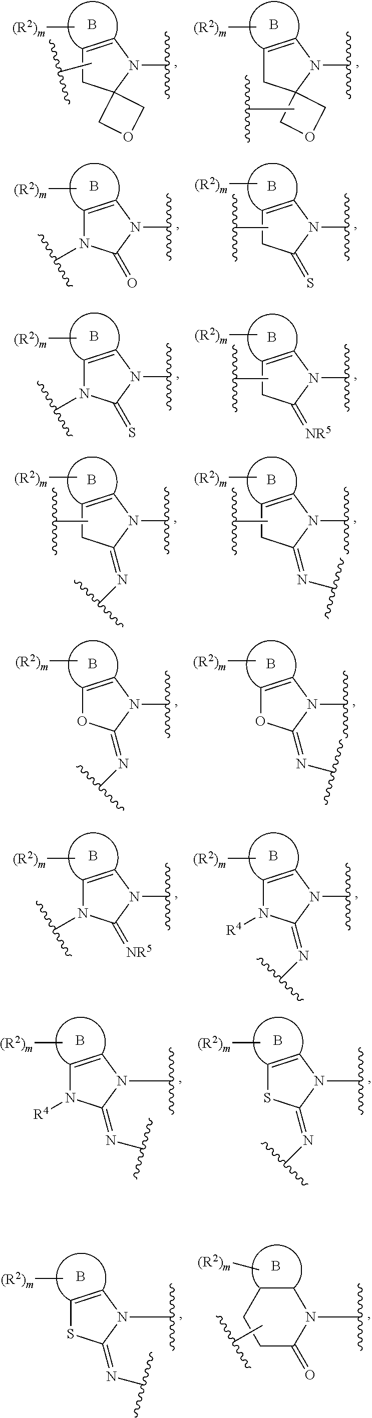

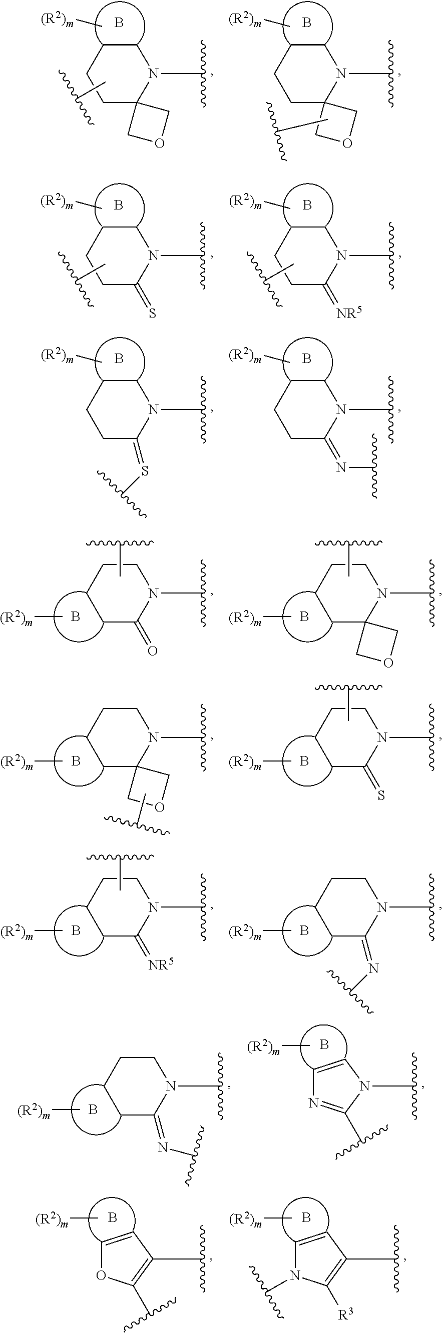

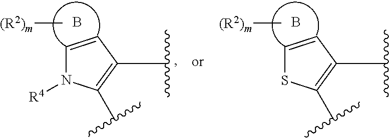

wherein
Ring B is a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
R³ is selected from hydrogen, halogen, —OR, —N(R)₂, or —SR;
each R⁴ is independently hydrogen, —R, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3 or 4;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

Where a point of attachment of —(R²)ₘ is depicted on Ring B, it is intended, and one of ordinary skill in the art would appreciate, that the point of attachment of —(R²)ₘ may be on Ring A and may also be at any available carbon or nitrogen atom on Ring A including the ring to which Ring B is fused. Where —R² is attached to a nitrogen atom bound to R⁴ or R⁵, R⁴ or R⁵ is absent and —R² takes the place of the R⁴ or R⁵ group. Where —R² is attached to a carbon atom bound to R³, R³ is absent and —R² takes the place of the R³ group.

In some embodiments, a compound of formula I-aa above is provided as a compound of formula I-aa′ or formula I-aa″:

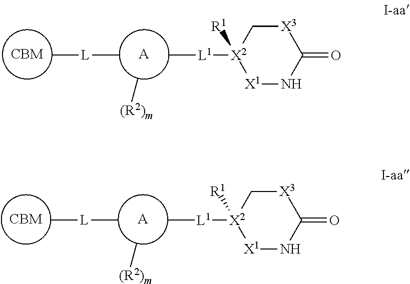

or a pharmaceutically acceptable salt thereof, wherein:
each of CBM, Ring A, L, L¹, R¹, R², X¹, X², X³, and m is as defined above.

In certain embodiments, the present invention provides a compound of Formula I, wherein DIM (e.g., LBM) is an E3 ubiquitin ligase (cereblon) binding moiety thereby forming a compound of formula I-cc:

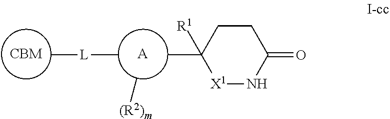

or a pharmaceutically acceptable salt thereof, wherein L and CBM are as defined above and described in embodiments herein, and wherein:
X¹ is a bivalent moiety selected from a covalent bond, —CH₂—, —C(O)—, —C(S)—, or

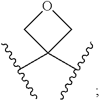 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each R² is independently hydrogen, —R, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R; Ring A is a bi- or tricyclic ring selected from

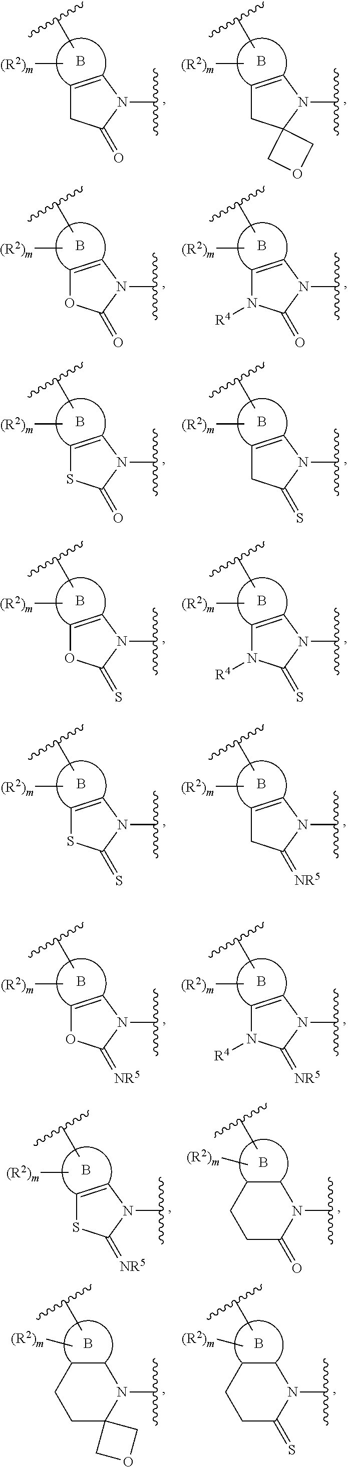

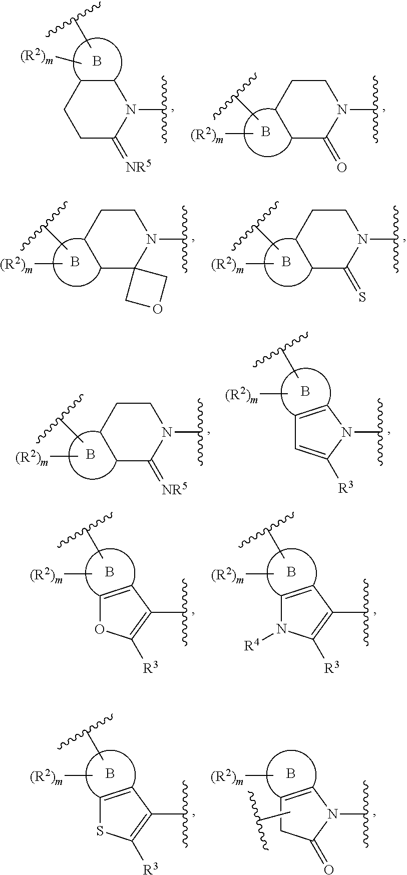

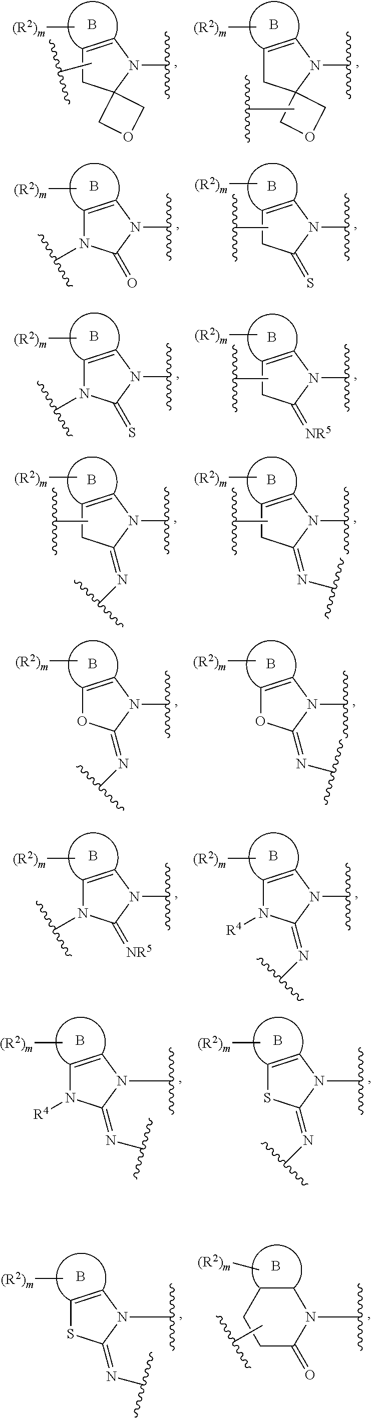

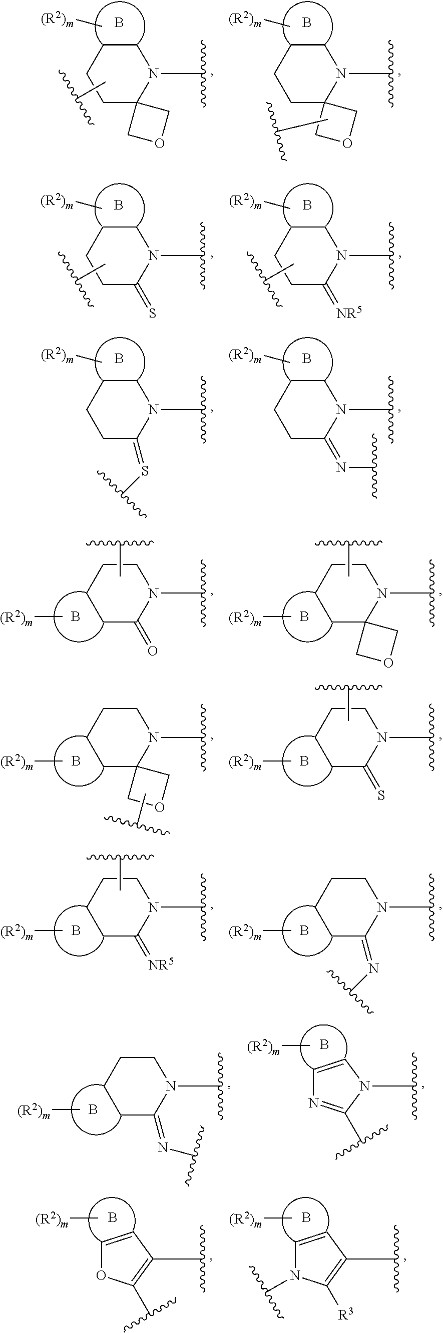

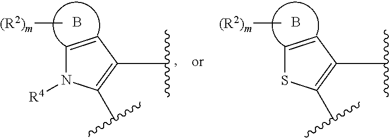

wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
R³ is selected from hydrogen, halogen, —OR, —N(R)₂, or —SR;
each R⁴ is independently hydrogen, —R, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
m is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

Where a point of attachment of —(R²)ₘ is depicted on Ring B, it is intended, and one of ordinary skill in the art would appreciate, that the point of attachment of —(R²)ₘ may be on Ring A and may also be at any available carbon or nitrogen atom on Ring A including the ring to which Ring B is fused. Where —R² is attached to a nitrogen atom bound to R⁴ or R⁵, R⁴ or R⁵ is absent and —R² takes the place of the R⁴ or R⁵ group. Where —R² is attached to a carbon atom bound to R³, R³ is absent and —R² takes the place of the R³ group.

In some embodiments, the compound of formula I-cc above is provided as a compound of formula I-cc′ or formula I-cc″:

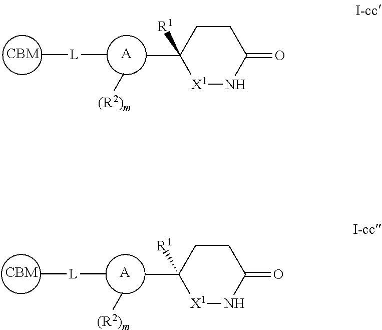

or a pharmaceutically acceptable salt thereof, wherein:
each of CBM, Ring A, L, R¹, R², X¹, and m is as defined above.

In certain embodiments, the present invention provides a compound of formula I, wherein DIM (e.g., LBM) is an E3 ubiquitin ligase (cereblon) binding moiety thereby forming a compound of formula I-dd:

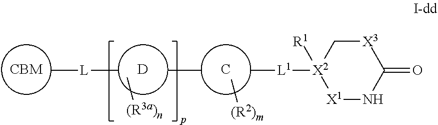

or a pharmaceutically acceptable salt thereof, wherein, L and CBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

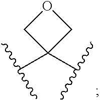 [SMILES: *C1(*)COC1]

X² is a carbon atom or silicon atom;
X³ is a bivalent moiety selected from —CR₂—, —NR—, —O—, —S—, or —Si(R²)—;
R¹ is hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
Ring C is a mono- or bicyclic ring selected from

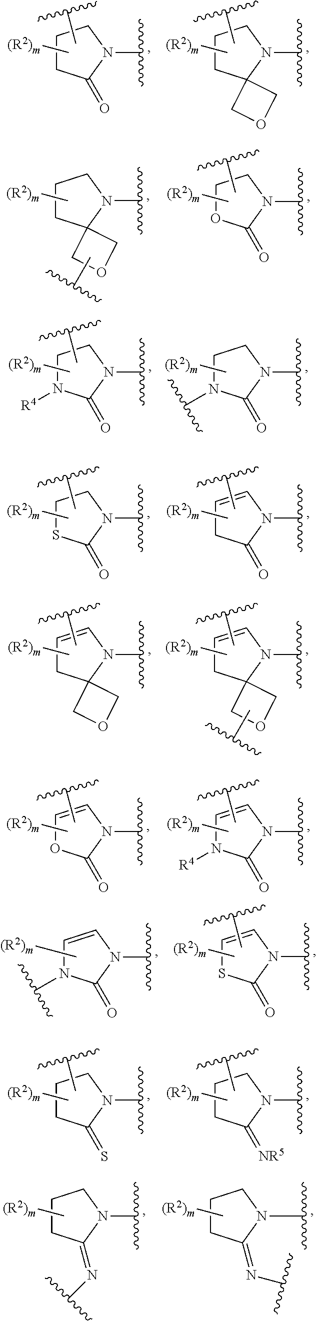

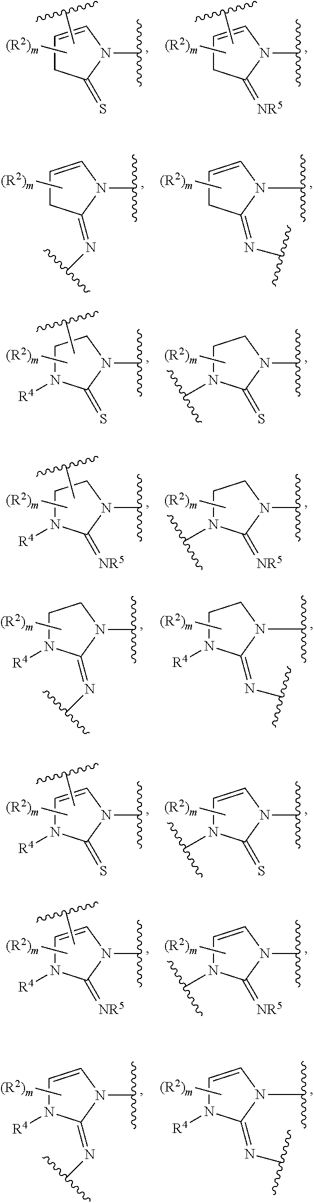

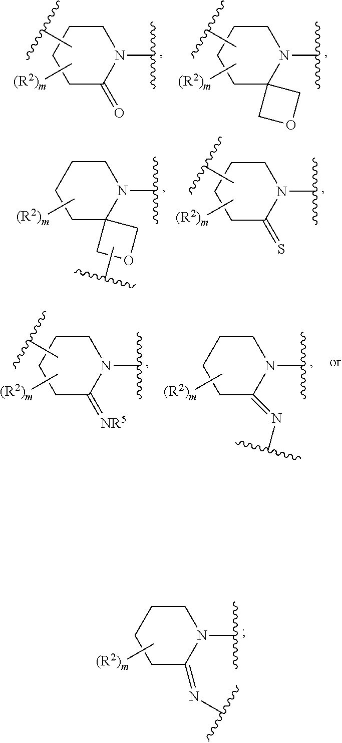

each of R² and R^{3a} is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring D is selected from a 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur; L is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3 or 4;
n is 0, 1, 2, 3 or 4;
p is 0 or 1, wherein when p is 0, the bond connecting Ring C and Ring D is connected to

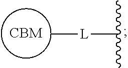

and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-dd above is provided as a compound of formula I-dd′ or formula I-dd″:

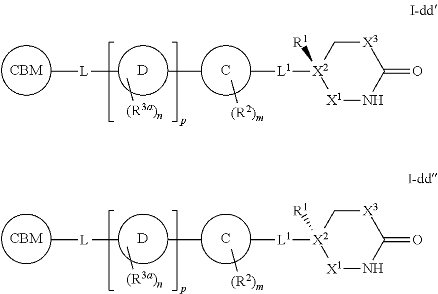

or a pharmaceutically acceptable salt thereof, wherein:
each of CBM, Ring C, Ring D, L, L¹, R¹, R², R^{3a}, X, X², X³, n, m, and p is as defined above.

In certain embodiments, the present invention provides a compound of Formula I, wherein DIM (e.g., LBM) is an E3 ubiquitin ligase (cereblon) binding moiety thereby forming a compound of formula I-ee:

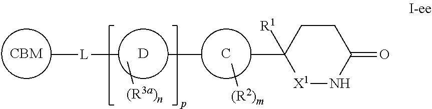

or a pharmaceutically acceptable salt thereof, wherein L and CBM are as defined above and described in embodiments herein, and wherein:
X¹ is a bivalent moiety selected from a covalent bond, —CH₂—, —C(O)—, —C(S)—, or

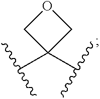 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
Ring C is a mono- or bicyclic ring selected from

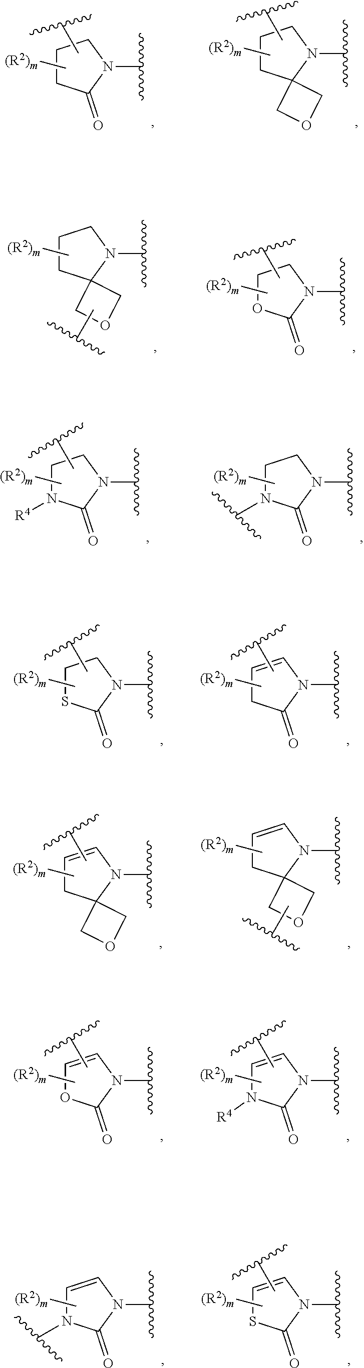

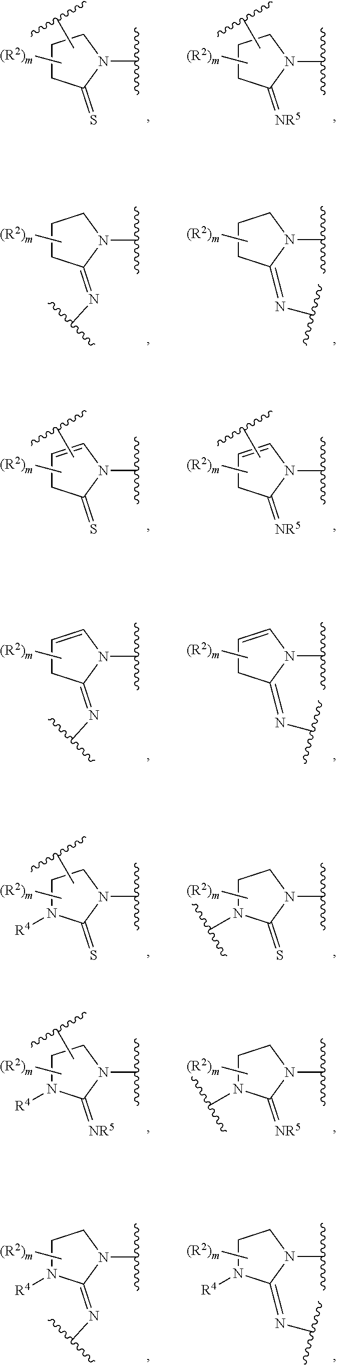

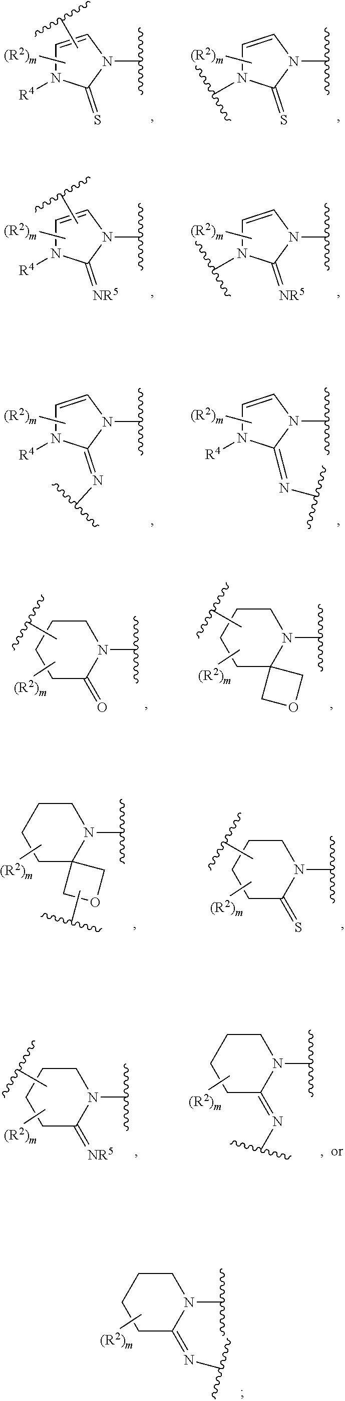

each of R² and R^{3a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
Ring D is selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
m is 0, 1, or 2;
n is 0, 1, 2, 3 or 4;
p is 0 or 1, wherein when p is 0, the bond connecting Ring C and Ring D is connected to

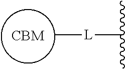

and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-ee above is provided as a compound of formula I-ee′ or formula I-ee″:

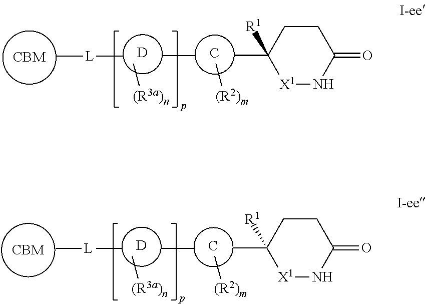

or a pharmaceutically acceptable salt thereof, wherein:
each of CBM, Ring C, Ring D, L, R¹, R², R^{3a} X¹, n, m, and p is as defined above.

In certain embodiments, the present invention provides a compound of formula I, wherein DIM (e.g., LBM) is an E3 ubiquitin ligase (cereblon) binding moiety thereby forming a compound of formula I-ff:

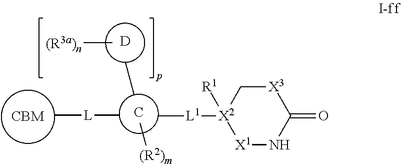

or a pharmaceutically acceptable salt thereof, wherein L and CBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

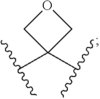 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
Ring C is a mono- or bicyclic ring selected from

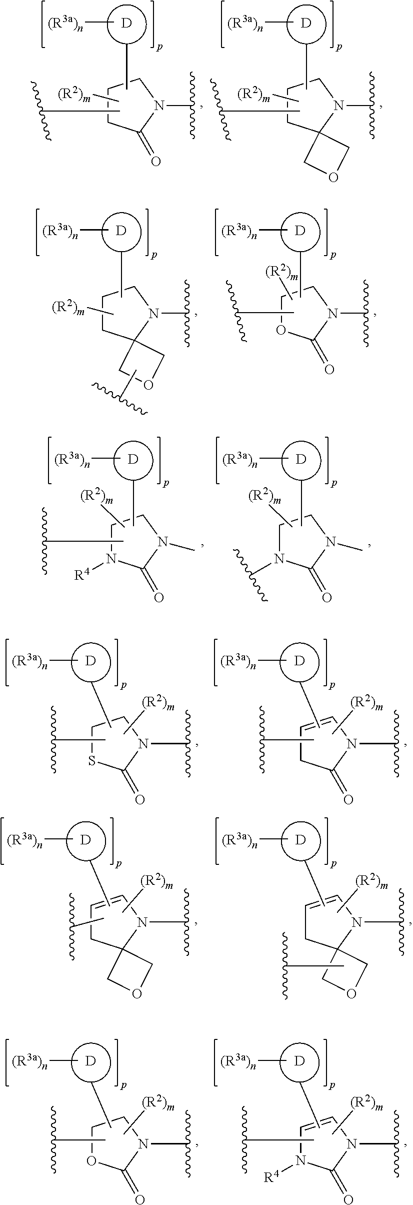

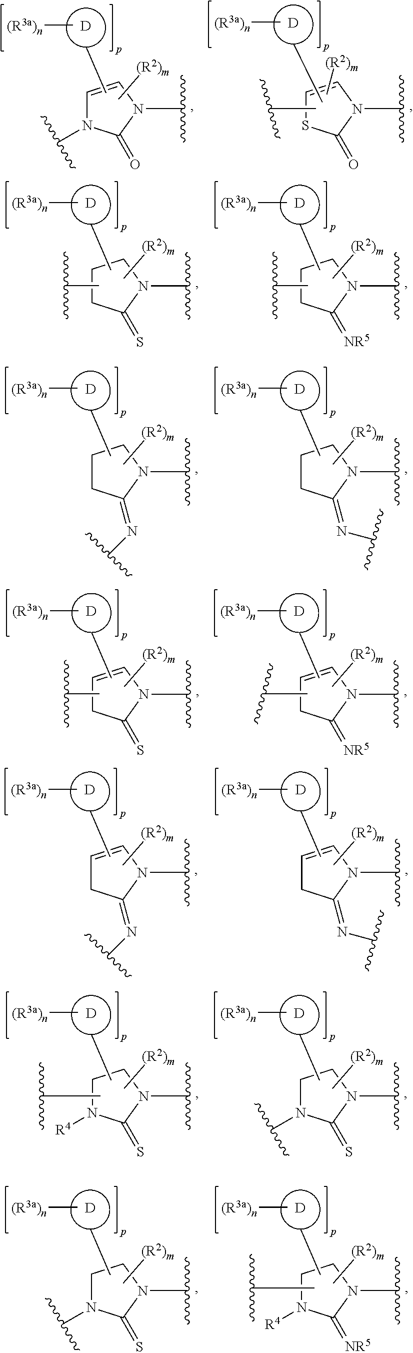

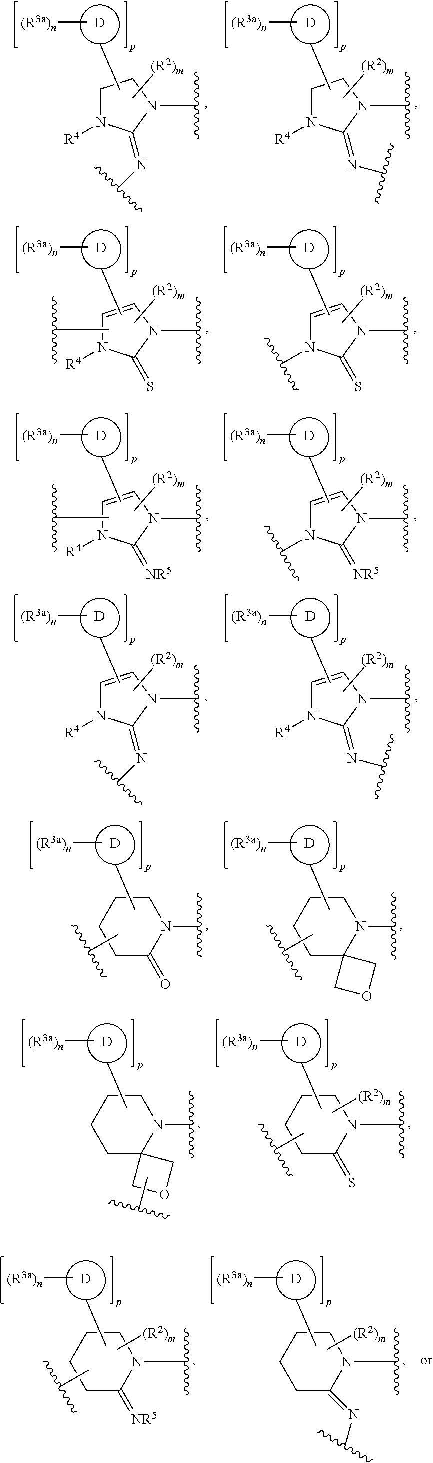

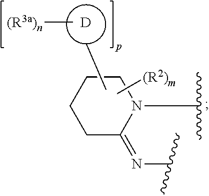

each or R² and R^{3a} is independently hydrogen, deuterium, —R, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring D is selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3 or 4;
n is 0, 1, 2, 3 or 4;
p is 0 or 1; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-ff above is provided as a compound of formula I-ff′ or formula I-ff″:

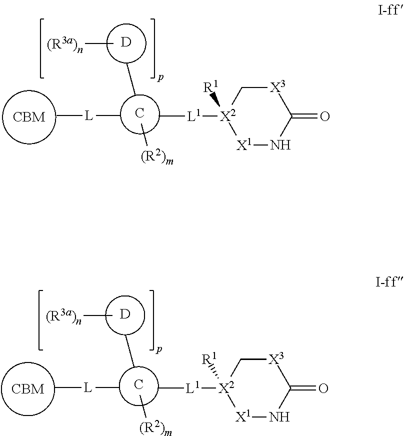

or a pharmaceutically acceptable salt thereof, wherein:
each of CBM, Ring C, Ring D, L, L¹, R¹, R², R^{3a}, X, X², X³, m, n, and p is as defined above.

In certain embodiments, the present invention provides a compound of Formula I, wherein DIM (e.g., LBM) is an E3 ubiquitin ligase (cereblon) binding moiety thereby forming a compound of formula I-gg:

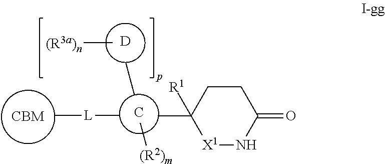

or a pharmaceutically acceptable salt thereof, wherein L and CBM are as defined above and described in embodiments herein, and wherein:
X¹ is a bivalent moiety selected from a covalent bond, —CH₂—, —C(O)—, —C(S)—, or

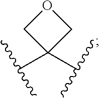 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
Ring C is a mono- or bicyclic ring selected from

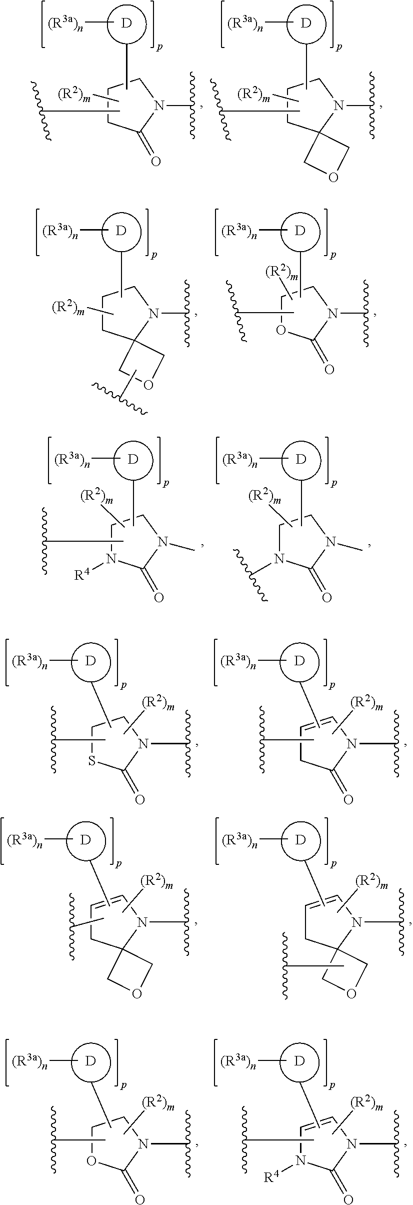

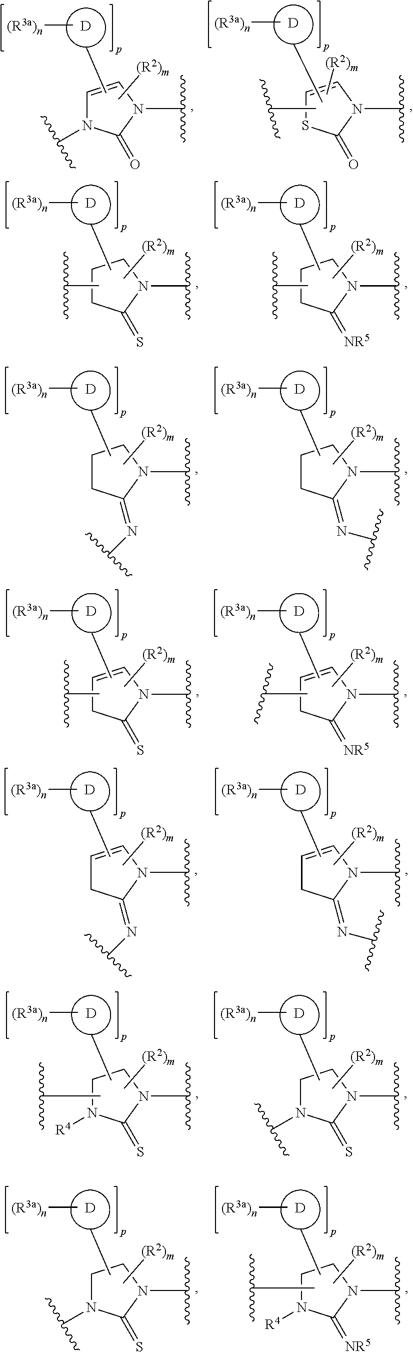

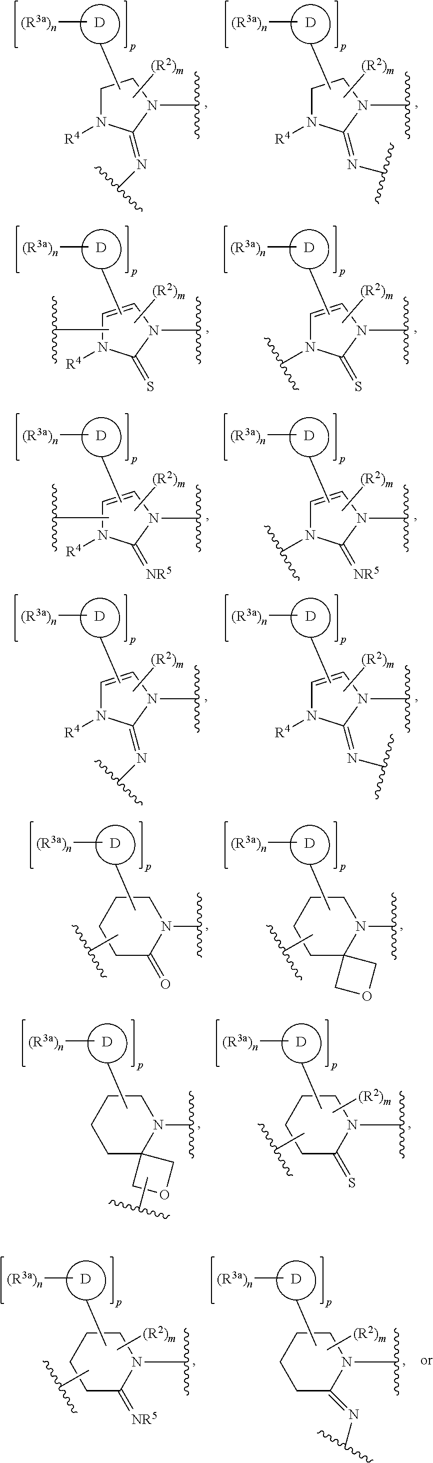

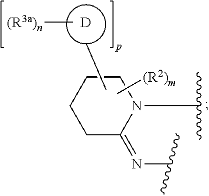

each of R², R^{3a}, and R⁴ is independently hydrogen, —R, halogen, —CN, —NO₂, —OR, —SR, —NR₂, —S(O)_{2R}, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
Ring D is selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
m is 0, 1, or 2;
n is 0, 1, 2, 3, or 4;
p is 0 or 1; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-gg above is provided as a compound of formula I-gg′ or formula I-gg″:

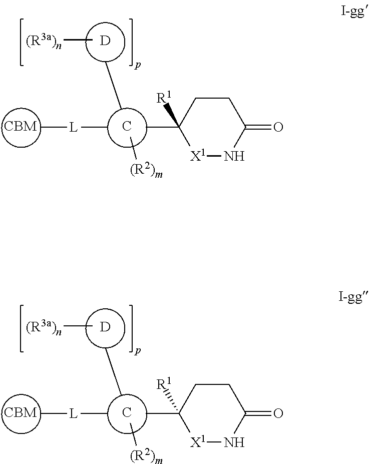

or a pharmaceutically acceptable salt thereof, wherein:
each of CBM, Ring C, Ring D, L, R¹, R², R^{3a}, X¹, m, n, and p is as defined above.

In certain embodiments, the present invention provides a compound of Formula I, wherein DIM (e.g., LBM) is an E3 ubiquitin ligase (cereblon) binding moiety thereby forming a compound of formula I-hh:

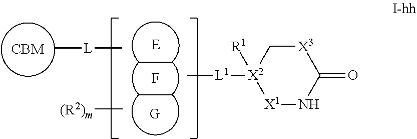

or a pharmaceutically acceptable salt thereof, wherein L and CBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

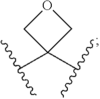 [SMILES: *C1(*)COC1]

X² is a carbon atom, nitrogen atom, or silicon atom;
X³ is a bivalent moiety selected from a covalent bond, —CR₂—, —NR—, —O—, —S—, or —SiR₂—;
R¹ is absent, hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur, wherein Ring E, Ring F, and Ring G is independently and optionally substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—; and
m is 0, 1, 2, 3, 4, 5, 6, 7, 8, 9, 10, 11, 12, 13, 14, 15, or 16.

Where a point of attachment of

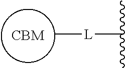

is depicted on Ring E, Ring F, or Ring G, it is intended, and one of ordinary skill in the art would appreciate, that the point of attachment of

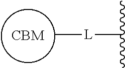

may be on any available carbon or nitrogen atom on Ring E, Ring F, or Ring G, including the ring to which Ring E or Ring G is fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G is fused to Ring F.

Where a point of attachment of

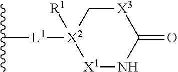

is depicted on Ring E, Ring F, or Ring G, it is intended, and one of ordinary skill in the art would appreciate, that the point of attachment of

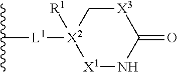

may be on any available carbon or nitrogen atom on Ring E, Ring F, or Ring G, including the carbon atom to which Ring E or Ring G is fused to Ring F.

In some embodiments, a compound of formula I-hh above is provided as a compound of formula I-hh′ or formula I-hh″:

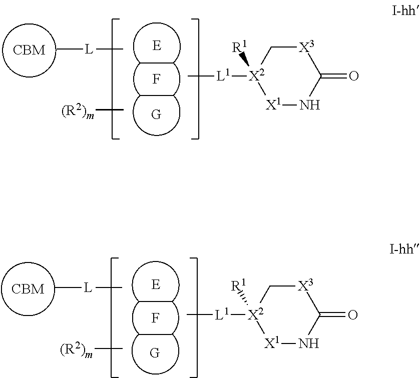

or a pharmaceutically acceptable salt thereof, wherein:
each of CBM, Ring E, Ring F, Ring G, L, L¹, R¹, R², X¹, X², X³, and m is as defined above.

In certain embodiments, the present invention provides a compound of Formula I, wherein DIM (e.g., LBM) is an E3 ubiquitin ligase (cereblon) binding moiety thereby forming a compound of formula I-hh-1 or I-hh-2:

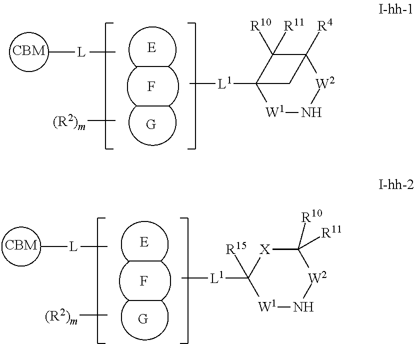

or a pharmaceutically acceptable salt thereof, wherein L and CBM are as defined above and described in embodiments herein, and wherein:
each R² is independently hydrogen, deuterium, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur, wherein Ring E, Ring F, and Ring G is independently and optionally substituted with 1-2 oxo groups;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3, 4, 5, 6, 7, 8, 9, 10, 11, 12, 13, 14, 15, or 16; and
R⁴, R¹⁰, R¹¹, R¹⁵, W¹, W², and X is as defined in WO 2019/099868, the entirety of each of which is herein incorporated by reference.

Where a point of attachment of

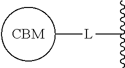

is depicted on Ring E, Ring F, or Ring G, it is intended, and one of ordinary skill in the art would appreciate, that the point of attachment of

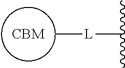

may be on any available carbon or nitrogen atom on Ring E, Ring F, or Ring G, including the ring to which Ring E or Ring G is fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G is fused to Ring F.

Where a point of attachment of

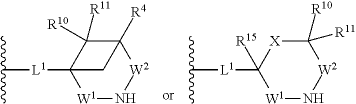

depicted on Ring E, Ring F, or Ring G, it is intended, and one of ordinary skill in the art would appreciate, that the point of attachment of

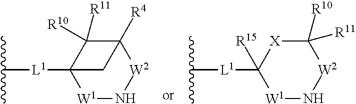

may be on any available carbon or nitrogen atom on Ring E, Ring F, or Ring G, including the carbon atom to which Ring E or Ring G is fused to Ring F.

In certain embodiments, the present invention provides a compound of Formula I, wherein LBM is an E3 ubiquitin ligase (cereblon) binding moiety thereby forming a compound of formula I-ii:

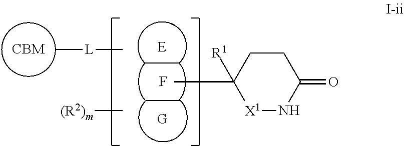

or a pharmaceutically acceptable salt thereof, wherein L and CBM are as defined above and described in embodiments herein, and wherein:
X¹ is a bivalent moiety selected from a covalent bond, —CH₂—, —C(O)—, —C(S)—, or

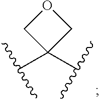 [SMILES: *C1(*)COC1]

R¹ is hydrogen, deuterium, halogen, —CN, —OR, —SR, —S(O)R, —S(O)₂R, —N(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —N(R)C(O)OR, —N(R)C(O)R, —N(R)C(O)N(R)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring E, Ring F, and Ring G is independently a fused ring selected from 6-membered aryl containing 0-3 nitrogens, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-3 heteroatoms independently selected from nitrogen, oxygen or sulfur, wherein Ring E, Ring F, and Ring G is independently and optionally substituted with 1-2 oxo groups; and
m is 0, 1, 2, 3, or 4.

Where a point of attachment of

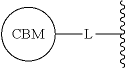

is depicted on Ring E, Ring F, or Ring G, it is intended, and one of ordinary skill in the art would appreciate, that the point of attachment of

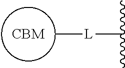

may be on any available carbon or nitrogen atom on Ring E, Ring F, or Ring G, including the ring to which Ring E or Ring G is fused to Ring F.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring E, Ring F, or Ring G including the carbon atom to which Ring E or Ring G is fused to Ring F.

In some embodiments, a compound of formula I-ii above is provided as a compound of formula I-ii′ or formula I-ii″:

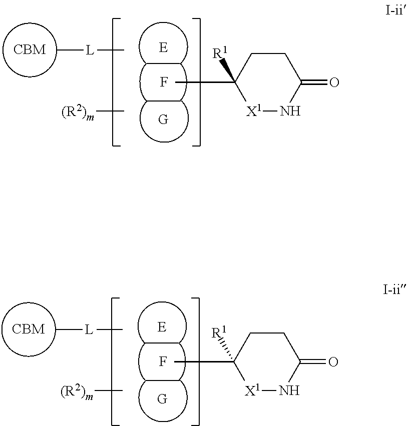

or a pharmaceutically acceptable salt thereof, wherein:
each of CBM, L, Ring E, Ring F, Ring G, L, R¹, R², X¹, and m is as defined above.

In certain embodiments, the present invention provides a compound of Formula I, wherein DIM (e.g., LBM) is an E3 ubiquitin ligase (cereblon) binding moiety thereby forming a compound of formula I-jj:

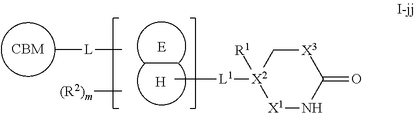

or a pharmaceutically acceptable salt thereof, wherein L and CBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

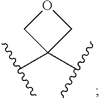 [SMILES: *C1(*)COC1]

X² is a carbon atom, nitrogen atom, or silicon atom;
X³ is a bivalent moiety selected from a covalent bond, —CR₂—, —NR—, —O—, —S—, or —SiR₂—;
R¹ is absent, hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring E is a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
Ring H is a fused ring selected from a 7-9 membered saturated or partially unsaturated carbocyclyl or heterocyclyl ring with 1-3 heteroatoms independently selected from boron, nitrogen, oxygen, silicon, or sulfur, wherein Ring E is optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
m is 0, 1, 2, 3, or 4.

Where a point of attachment of

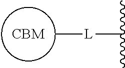

is depicted on Ring E or Ring H, it is intended, and one of ordinary skill in the art would appreciate, that the point of attachment of

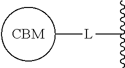

may be on any available carbon or nitrogen atom on Ring E or Ring H including the carbon atom to which Ring E and Ring H are fused.

Where a point of attachment of —(R²)ₘ is depicted on Ring E and Ring H, it is intended, and one of ordinary skill in the art would appreciate, that the point of attachment of —(R²)ₘ may be on any available carbon or nitrogen atom on Ring E or Ring H including the carbon atom to which Ring E and Ring H are fused.

Where a point of attachment of

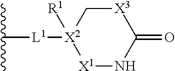

is depicted on Ring E and Ring H, it is intended, and one of ordinary skill in the art would appreciate, that the point of attachment of

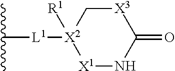

may be on any available carbon or nitrogen atom on Ring E or Ring H including the carbon atom to which Ring E and Ring H are fused.

In some embodiments, a compound of formula I-jj above is provided as a compound of formula I-jj′ or formula I-jj″:

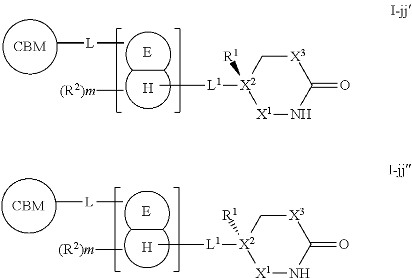

or a pharmaceutically acceptable salt thereof, wherein:
each of CBM, Ring E, Ring H, L, L, R¹, R², X¹, X², X³, and m is as defined above.

In certain embodiments, the present invention provides a compound of Formula I, wherein LBM is an E3 ubiquitin ligase (cereblon) binding moiety thereby forming a compound of formula I-kk:

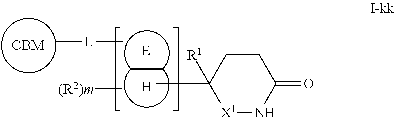

or a pharmaceutically acceptable salt thereof, wherein L and CBM are as defined above and described in embodiments herein, and wherein:
X¹ is a bivalent moiety selected from a covalent bond, —CH₂—, —C(O)—, —C(S)—, or

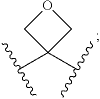 [SMILES: *C1(*)COC1]

R¹ is hydrogen, deuterium, halogen, —CN, —OR, —SR, —S(O)R, —S(O)₂R, —N(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —N(R)C(O)OR, —N(R)C(O)R, —N(R)C(O)N(R)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring E is a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
Ring H is a ring selected from a 7-9 membered saturated or partially unsaturated carbocyclyl or heterocyclyl ring with 1-3 heteroatoms independently selected from boron, nitrogen, oxygen, silicon, or sulfur,
   wherein Ring E is optionally further substituted with 1-2 oxo groups; and
m is 0, 1, 2, 3, or 4.

Where a point of attachment of

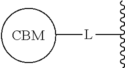

is depicted on Ring E or Ring H, it is intended, and one of ordinary skill in the art would appreciate, that the point of attachment of

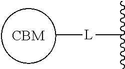

may be on any available carbon or nitrogen atom on Ring E or Ring H including the carbon atom to which Ring E and Ring H are fused.

Where a point of attachment of —(R²)ₘ is depicted on Ring E and Ring H, it is intended, and one of ordinary skill in the art would appreciate, that the point of attachment of —(R²)ₘ may be on any available carbon or nitrogen atom on Ring E or Ring H including the carbon atom to which Ring E and Ring H are fused.

Where a point of attachment of

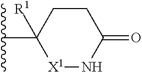

is depicted on Ring E and Ring H, it is intended, and one of ordinary skill in the art would appreciate, that the point of attachment of

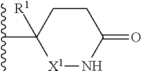

may be on any available carbon or nitrogen atom on Ring E or Ring H including the carbon atom to which Ring E and Ring H are fused.

In some embodiments, a compound of formula I-kk above is provided as a compound of formula I-kk′ or formula I-kk″:

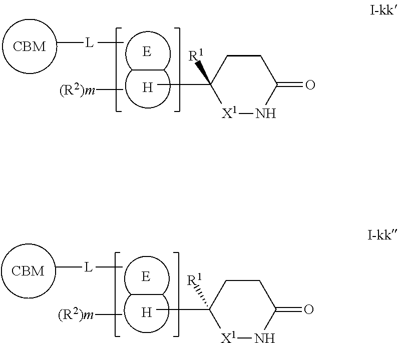

or a pharmaceutically acceptable salt thereof, wherein:
each of CBM, Ring E, Ring H, L, R¹, R², X¹, and m is as defined above.

In certain embodiments, the present invention provides a compound of Formula I, wherein DIM (e.g., LBM) is an E3 ubiquitin ligase (cereblon) binding moiety thereby forming a compound of formula I-ll:

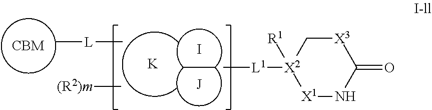

or a pharmaceutically acceptable salt thereof, wherein L and CBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

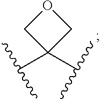 [SMILES: *C1(*)COC1]

X² is a carbon atom, nitrogen atom, or silicon atom;
X³ is a bivalent moiety selected from a covalent bond, —CR₂—, —NR—, —O—, —S—, or —SiR₂—;
R¹ is absent, hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring I and J is independently a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
Ring K is a fused ring selected from a 5-12 membered saturated or partially unsaturated carbocyclyl or heterocyclyl ring with 1-3 heteroatoms independently selected from boron, nitrogen, oxygen, silicon, or sulfur, wherein Ring H is optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—; and
m is 0, 1, 2, 3, or 4.

Where a point of attachment of

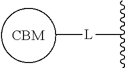

is depicted on Ring I, Ring J, and Ring K, it is intended, and one of ordinary skill in the art would appreciate, that the point of attachment of

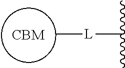

may be on any available carbon or nitrogen atom on Ring I, Ring J, or Ring K, including the carbon atom to which Ring I, Ring J, and Ring K are fused.

Where a point of attachment of —(R²)ₘ is depicted on Ring I, Ring J, and Ring K, it is intended, and one of ordinary skill in the art would appreciate, that the point of attachment of —(R²)ₘ may be on any available carbon or nitrogen atom on Ring I, Ring J, or Ring K, including the carbon atom to which Ring I, Ring J, and Ring K are fused.

Where a point of attachment of

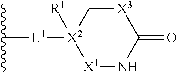

is depicted on Ring I, Ring J, and Ring K, it is intended, and one of ordinary skill in the art would appreciate, that the point of attachment of

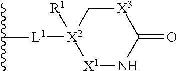

may be on any available carbon or nitrogen atom on Ring I, Ring J, or Ring K, including the carbon atom to which Ring I, Ring J, and Ring K are fused.

In some embodiments, a compound of formula I-ll above is provided as a compound of formula I-ll′ or formula I-ll″:

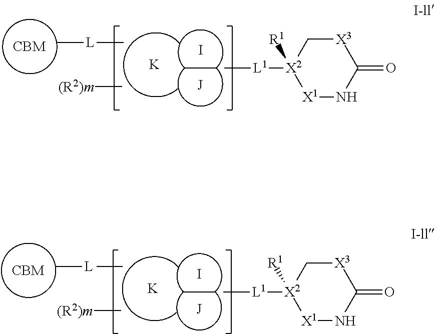

or a pharmaceutically acceptable salt thereof, wherein:
each of CBM, Ring I, Ring J, Ring K, L, L¹, R¹, R², X¹, X², X³, and m is as defined above.

In certain embodiments, the present invention provides a compound of formula I-mm:

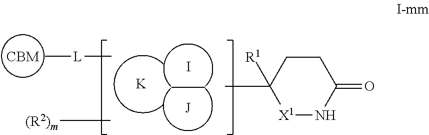

or a pharmaceutically acceptable salt thereof, wherein L and CBM are as defined above and described in embodiments herein, and wherein:
X¹ is a bivalent moiety selected from a covalent bond, —CH₂—, —C(O)—, —C(S)—, or

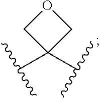 [SMILES: *C1(*)COC1]

R¹ is hydrogen, deuterium, halogen, —CN, —OR, —SR, —S(O)R, —S(O)₂R, —N(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —N(R)C(O)OR, —N(R)C(O)R, —N(R)C(O)N(R)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring I and J is independently a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
Ring K is a fused ring selected from a 5-12 membered saturated or partially unsaturated carbocyclyl or heterocyclyl ring with 1-3 heteroatoms independently selected from boron, nitrogen, oxygen, silicon, or sulfur, wherein Ring H is optionally further substituted with 1-2 oxo groups; and
m is 0, 1, 2, 3, or 4.

Where a point of attachment of

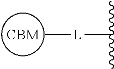

is depicted on Ring I, Ring J, and Ring K, it is intended, and one of ordinary skill in the art would appreciate, that the point of attachment of

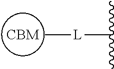

may be on any available carbon or nitrogen atom on Ring I, Ring J, or Ring K, including the carbon atom to which Ring I, Ring J, and Ring K are fused.

Where a point of attachment of —(R²)ₘ is depicted on Ring I, Ring J, and Ring K, it is intended, and one of ordinary skill in the art would appreciate, that the point of attachment of —(R²)ₘ may be on any available carbon or nitrogen atom on Ring I, Ring J, or Ring K, including the carbon atom to which Ring I, Ring J, and Ring K are fused.

Where a point of attachment of

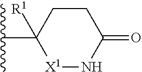

is depicted on Ring I, Ring J, and Ring K, it is intended, and one of ordinary skill in the art would appreciate, that the point of attachment of

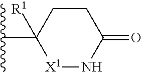

may be on any available carbon or nitrogen atom on Ring I, Ring J, or Ring K, including the carbon atom to which Ring I, Ring J, and Ring K are fused.

In some embodiments, a compound of formula I-mm above is provided as a compound of formula I-mm′ or formula I-mm″:

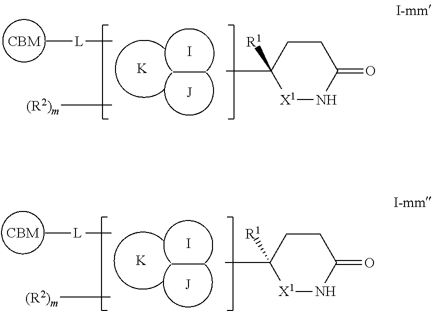

or a pharmaceutically acceptable salt thereof, wherein:
each of CBM, Ring I, Ring J, Ring K, L, R¹, R², X¹, and m is as defined above.

As described above, in another aspect, the present invention provides a compound of Formula I-nn:

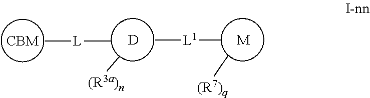

or a pharmaceutically acceptable salt thereof, wherein:
Ring M is selected from

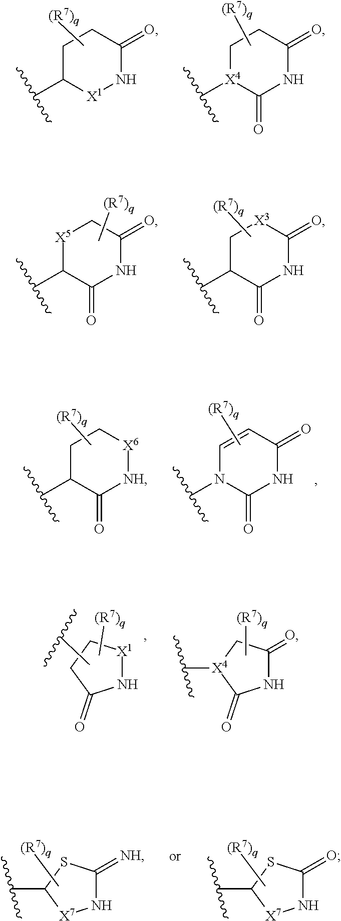

each of X¹, X⁶, and X⁷ is independently a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

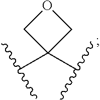 [SMILES: *C1(*)COC1]

each of X³ and X⁵ is independently a bivalent moiety selected from a covalent bond, —CR₂—, —NR—, —O—, —S—, or —SiR₂—;
X⁴ is a trivalent moiety selected from

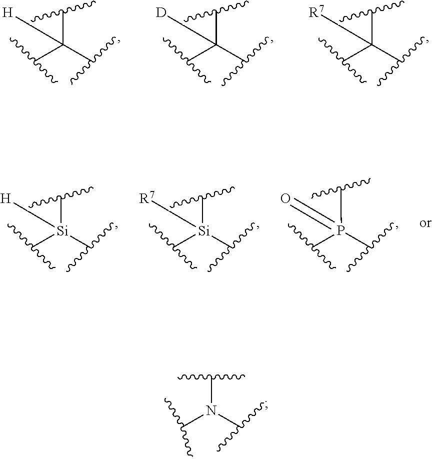

each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R^{3a} is independently hydrogen, deuterium, —R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁷ is independently hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic; or
R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, or sulfur;
   two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, or sulfur;
   two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, or sulfur, or a 7-13 membered saturated, partially unsaturated, bridged heterocyclic ring, or a spiro heterocyclic ring having 1-3 heteroatoms, independently selected from boron, nitrogen, oxygen, silicon, or sulfur;
Ring D is selected from 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
n is 0, 1, 2, 3, or 4; and
q is 0, 1, 2, 3, or 4.

In some embodiments, a compound of formula I-nn, wherein X⁴ is nitrogen, to provide a compound of formula I-nn-1:

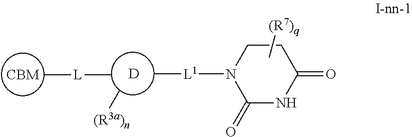

or a pharmaceutically acceptable salt thereof, wherein:

As defined above and described herein, each of X¹, X⁶, and X⁷ is independently a bivalent moiety selected from a covalent bond, —CH₂—, —C(R)₂—, —C(O)—, —C(S)—, —CH(R)—, —CH(CF₃)—, —P(O)(OR)—, —P(O)(R)—, —P(O)(NR₂)—, —S(O)—, —S(O)₂—, or

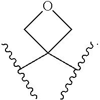 [SMILES: *C1(*)COC1]

In some embodiments, each of X¹, X⁶, and X⁷ is independently a covalent bond. In some embodiments, each of X¹, X⁶, and X⁷ is independently —CH₂—. In some embodiments, each of X¹, X⁶, and X⁷ is independently —CR₂—. In some embodiments, each of X¹, X⁶, and X⁷ is independently —C(O)—. In some embodiments, each of X¹, X⁶, and X⁷ is independently —C(S)—. In some embodiments, each of X¹, X⁶, and X⁷ is independently —CH(R)—. In some embodiments, each of X¹, X⁶, and X⁷ is independently —CH(CF₃)—. In some embodiments, each of X¹, X⁶, and X⁷ is independently —P(O)(OR)—. In some embodiments, each of X¹, X⁶, and X⁷ is independently —P(O)(R)—. In some embodiments, each of X¹, X⁶, and X⁷ is independently —P(O)NR₂—. In some embodiments, each of X¹, X⁶, and X⁷ is independently —S(O)—. In some embodiments, each of X¹, X⁶, and X⁷ is independently —S(O)₂—. In some embodiments, each of X¹, X⁶, and X⁷ is independently

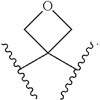 [SMILES: *C1(*)COC1]

In some embodiments, each of X¹, X⁶, and X⁷ is independently selected from those depicted in Table 1 below.

As defined above and described herein, X² is a carbon atom, nitrogen atom, or silicon atom.

In some embodiments, X² is a carbon atom. In some embodiments, X² is a nitrogen atom. In some embodiments, X² is a silicon atom.

In some embodiments, X² is selected from those depicted in Table 1 below.

As defined above and described herein, X³ is a bivalent moiety selected from —CH₂—, —CR₂—, —NR—, —CF₂—, —CHF—, —S—, —CH(R)—, —SiR₂—, or —O—.

In some embodiments, each of X³ and X⁵ is independently —CH₂—. In some embodiments, each of X³ and X⁵ is independently —CR₂—. In some embodiments, each of X³ and X⁵ is independently —NR—. In some embodiments, each of X³ and X⁵ is independently —CF₂—. In some embodiments, each of X³ and X⁵ is independently —CHF—. In some embodiments, each of X³ and X⁵ is independently —S—. In some embodiments, each of X³ and X⁵ is independently —CH(R)—. In some embodiments, each of X³ and X⁵ is independently —SiR₂—. In some embodiments, each of X³ and X⁵ is independently —O—.

In some embodiments, each of X³ and X⁵ is independently selected from those depicted in Table 1 below.

As defined above and described herein, X⁴ is a trivalent moiety selected from

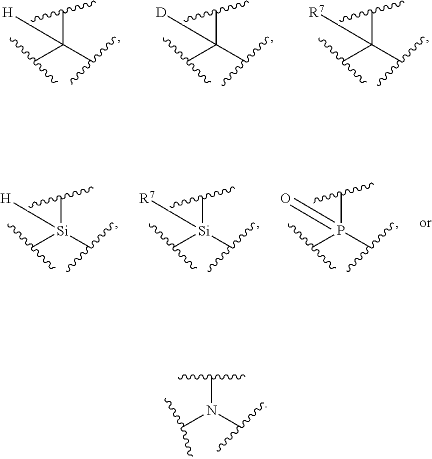

In some embodiments, X⁴ is

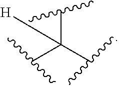 [SMILES: *C(*)*]

In some embodiments, X⁴ is

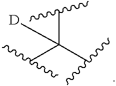 [SMILES: *C(*)(*)[2H]]

In some embodiments, X⁴ is

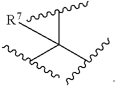

In some embodiments, X⁴ is

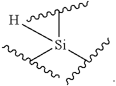 [SMILES: *[SiH](*)*]

In some embodiments, X⁴ is

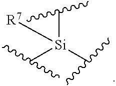

In some embodiments, X⁴ is

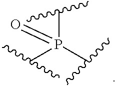 [SMILES: *P(*)(*)=O]

In some embodiments, X⁴ is

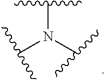 [SMILES: *N(*)*]

In some embodiments, X⁴ is selected from those depicted in Table 1 below.

As defined above and described herein, R¹ is hydrogen, deuterium, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, an optionally substituted C₁₋₄ aliphatic, or R¹ and X¹ or X⁴ are taken together with their intervening atoms to form a 5-7 membered saturated, partially unsaturated, carbocyclic ring or heterocyclic ring having 1-3 heteroatoms, independently selected from nitrogen, oxygen, or sulfur.

In some embodiments, R¹ is hydrogen. In some embodiments, R¹ is deuterium. In some embodiments, R¹ is halogen. In some embodiments, R¹ is —CN. In some embodiments, R¹ is —OR. In some embodiments, R¹ is —SR. In some embodiments, R¹ is —S(O)R. In some embodiments, R¹ is —S(O)₂R. In some embodiments, R¹ is —NR₂. In some embodiments, R¹ is —P(O)(OR)₂. In some embodiments, R¹ is —P(O)(NR₂)OR. In some embodiments, R¹ is —P(O)(NR₂)₂. In some embodiments, R¹ is —Si(OH)₂R. In some embodiments, R¹ is —Si(OH)R₂. In some embodiments, R¹ is —SiR₃. In some embodiments, R¹ is an optionally substituted C₁₋₄ aliphatic. In some embodiments, R¹ and X¹ or X⁴ are taken together with their intervening atoms to form a 5-7 membered saturated, partially unsaturated, carbocyclic ring or heterocyclic ring having 1-3 heteroatoms, independently selected from nitrogen, oxygen, or sulfur.

In some embodiments, R¹ is selected from those depicted in Table 1 below.

As defined above and described herein, each R is independently hydrogen, deuterium, or an optionally substituted group selected from C₁₋₆ aliphatic, phenyl, a 4-7 membered saturated or partially unsaturated heterocyclic having 1-3 heteroatoms independently selected from boron, nitrogen, oxygen, silicon, and sulfur, and a 5-6 membered heteroaryl ring having 1-4 heteroatoms independently selected from boron, nitrogen, oxygen, silicon, and sulfur, or two R groups on the same nitrogen are taken together with their intervening atoms to form a 4-7 membered saturated, partially unsaturated, or heteroaryl ring having 0-3 heteroatoms, in addition to the nitrogen, independently selected from boron, nitrogen, oxygen, silicon, and sulfur.

In some embodiments, R is hydrogen. In some embodiments, R is deuterium. In some embodiments, R is optionally substituted C₁₋₆ aliphatic. In some embodiments, R is optionally substituted phenyl. In some embodiments, R is optionally substituted 4-7 membered saturated or partially unsaturated heterocyclic having 1-3 heteroatoms independently selected from boron, nitrogen, oxygen, silicon, and sulfur. In some embodiments, R is optionally substituted 5-6 membered heteroaryl ring having 1-4 heteroatoms independently selected from boron, nitrogen, oxygen, silicon, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from boron, nitrogen, oxygen, silicon, and sulfur.

In some embodiments, R is selected from those depicted in Table 1 below.

As defined above and described herein, each of R² and R^{3a} is independently hydrogen, deuterium, —R⁶, halogen, —CN, —NO₂, —OR, —Si(OH)₂R, —Si(OH)R₂, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)NR₂, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R.

In some embodiments, R² and/or R^{3a} is hydrogen. In some embodiments, R² and/or R^{3a} is deuterium. In some embodiments, R² and/or R^{3a} is —R⁶. In some embodiments, R² and/or R^{3a} is halogen. In some embodiments, R² and/or R^{3a} is —CN. In some embodiments, R² and/or R^{3a} is —NO₂. In some embodiments, R² and/or R^{3a} is —OR. In some embodiments, R² and/or R^{3a} is —Si(OH)₂R. In some embodiments, R² and/or R^{3a} is —Si(OH)R₂. In some embodiments, R² and/or R^{3a} is —SR. In some embodiments, R² and/or R^{3a} is —NR₂. In some embodiments, R² and/or R^{3a} is —SiR₃. In some embodiments, R² and/or R^{3a} is —S(O)₂R. In some embodiments, R² and/or R^{3a} is —S(O)₂NR₂. In some embodiments, R² and/or R^{3a} is —S(O)R. In some embodiments, R² and/or R^{3a} is —C(O)R. In some embodiments, R² and/or R^{3a} is —C(O)OR. In some embodiments, R² and/or R^{3a} is —C(O)NR₂. In some embodiments, R² and/or R^{3a} is —C(O)N(R)OR. In some embodiments, R² and/or R^{3a} is —C(R)₂N(R)C(O)R. In some embodiments, R² and/or R^{3a} is —C(R)₂N(R)C(O)NR₂. In some embodiments, R² and/or R^{3a} is —OC(O)R. In some embodiments, R² and/or R^{3a} is —OC(O)NR₂. In some embodiments, R² and/or R^{3a} is —OP(O)R₂. In some embodiments, R² and/or R^{3a} is —OP(O)(OR)₂. In some embodiments, R² and/or R^{3a} is —OP(O)(OR)NR₂. In some embodiments, R² and/or R^{3a} is —OP(O)(NR₂)₂—. In some embodiments, R² and/or R^{3a} is —N(R)C(O)OR. In some embodiments, R² and/or R^{3a} is —N(R)C(O)R. In some embodiments, R² and/or R^{3a} is —N(R)C(O)NR₂. In some embodiments, R² and/or R^{3a} is —NP(O)R₂. In some embodiments, R² and/or R^{3a} is —N(R)P(O)(OR)₂. In some embodiments, R² and/or R^{3a} is —N(R)P(O)(OR)NR₂. In some embodiments, R² and R^{3a} is independently —N(R)P(O)(NR₂)₂. In some embodiments, R² and/or R^{3a} is —N(R)S(O)₂R.

In some embodiments, R² and R^{3a} is independently —OH. In some embodiments, R² and/or R^{3a} is —NH₂. In some embodiments, R² and/or R^{3a} is —CH₂NH₂. In some embodiments, R² and/or R^{3a} is —CH₂NHCOMe. In some embodiments, R² and/or R^{3a} is —CH₂NHCONHMe. In some embodiments, R² and/or R^{3a} is —NHCOMe. In some embodiments, R² and/or R^{3a} is —NHCONHEt. In some embodiments, R² and/or R^{3a} is —SiMe₃. In some embodiments, R² and/or R^{3a} is —SiMe₂OH. In some embodiments, R² and/or R^{3a} is —SiMe(OH)₂. In some embodiments R² and/or R^{3a} is

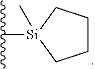 [SMILES: *[Si]1(C)CCCC1]

In some embodiments, R² and/or R^{3a} is Br. In some embodiments, R² and/or R^{3a} is Cl. In some embodiments, R² and/or R^{3a} is F. In some embodiments, R² and/or R^{3a} is Me. In some embodiments, R² and/or R^{3a} is —NHMe. In some embodiments, R² and/or R^{3a} is —NMe₂. In some embodiments, R² and/or R^{3a} is —NHCO₂Et. In some embodiments, R² and/or R^{3a} is —CN. In some embodiments, R² and/or R^{3a} is —CH₂Ph. In some embodiments, R² and/or R^{3a} is —NHCO₂tBu. In some embodiments, R² and/or R^{3a} is —CO₂tBu. In some embodiments, R² and/or R^{3a} is —OMe. In some embodiments, R² and/or R^{3a} is —CF₃.

In some embodiments, R² or R^{3a} is selected from those depicted in Table 1 below.

As defined above and described herein, R³ is hydrogen, deuterium, halogen, —CN, —NO₂, —OR, —NR₂, —SR, —S(O)₂R, —S(O)₂NR₂, —S(O)R, —C(O)R, —C(O)OR, —C(O)NR₂, —C(O)NR(OR), —OC(O)R, —OC(O)NR₂, —OP(O)(OR)₂, —OP(O)(NR₂)₂, —OP(O)(OR)NR₂, —N(R)C(O)R, —N(R)C(O)OR, —N(R)C(O)NR₂, —N(R)S(O)₂R, —N(R)S(O)₂NR₂, —N(R)P(O)(OR)₂, —N(R)P(O)(OR)NR₂, —P(O)(OR)₂, —P(O)(NR₂)OR, —P(O)(NR₂)₂, —Si(OH)₂R, —Si(OH)(R)₂, or —Si(R)₃.

In some embodiments, R³ is hydrogen. In some embodiments, R³ is deuterium. In some embodiments, R³ is halogen. In some embodiments, R³ is —CN. In some embodiments, R³ is —NO₂. In some embodiments, R³ is —OR. In some embodiments, R³ is —NR₂. In some embodiments, R³ is —SR. In some embodiments, R³ is —S(O)₂R. In some embodiments, R³ is —S(O)₂NR₂. In some embodiments, R³ is —S(O)R. In some embodiments, R³ is —C(O)R. In some embodiments, R³ is —C(O)OR. In some embodiments, R³ is —C(O)NR₂. In some embodiments, R³ is —C(O)NR(OR). In some embodiments, R³ is —OC(O)R. In some embodiments, R³ is —OC(O)NR₂. In some embodiments, R³ is —OP(O)(OR)₂. In some embodiments, R³ is —OP(O)(NR₂)₂. In some embodiments, R³ is —OP(O)(OR)NR₂. In some embodiments, R³ is —N(R)C(O)R. In some embodiments, R³ is —N(R)C(O)OR. In some embodiments, R³ is —N(R)C(O)NR₂. In some embodiments, R³ is —N(R)S(O)₂R. In some embodiments, R³ is —N(R)S(O)₂NR₂. In some embodiments, R³ is —N(R)P(O)(OR)₂. In some embodiments, R³ is —N(R)P(O)(OR)NR₂. In some embodiments, R³ is —P(O)(OR)₂. In some embodiments, R³ is —P(O)(NR₂)OR. In some embodiments, R³ is —P(O)(NR₂)₂. In some embodiments, R³ is —Si(OH)₂R. In some embodiments, R³ is —Si(OH)(R)₂. In some embodiments, R³ is —Si(R)₃.

In some embodiments, R³ is methyl. In some embodiments, R³ is —OCH₃. In some embodiments, R³ is chloro.

In some embodiments, R³ is selected from those depicted in Table 1.

As defined above and described herein, each R⁴ is independently hydrogen, deuterium, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —P(O)(OR)₂, —P(O)(NR₂)OR, or —P(O)(NR₂)₂.

In some embodiments, R⁴ is hydrogen. In some embodiments, R⁴ is —R⁶. In some embodiments, R⁴ is halogen. In some embodiments, R⁴ is —CN. In some embodiments, R⁴ is —NO₂. In some embodiments, R⁴ is —OR. In some embodiments, R⁴ is —SR. In some embodiments, R⁴ is —NR₂. In some embodiments, R⁴ is —S(O)₂R. In some embodiments, R⁴ is —S(O)₂NR₂. In some embodiments, R⁴ is —S(O)R. In some embodiments, R⁴ is —C(O)R. In some embodiments, R⁴ is —C(O)OR. In some embodiments, R⁴ is —C(O)NR₂. In some embodiments, R⁴ is —C(O)N(R)OR. In some embodiments, R⁴ is —OC(O)R. In some embodiments, R⁴ is —OC(O)NR₂. In some embodiments, R⁴ is —N(R)C(O)OR. In some embodiments, R⁴ is —N(R)C(O)R. In some embodiments, R⁴ is —N(R)C(O)NR₂. In some embodiments, R⁴ is —N(R)S(O)₂R. In some embodiments, R⁴ is —P(O)(OR)₂. In some embodiments, R⁴ is —P(O)(NR₂)OR. In some embodiments, R⁴ is —P(O)(NR₂)₂.

In some embodiments, R⁴ is methyl. In some embodiments, R⁴ is ethyl. In some embodiments, R⁴ is cyclopropyl.

In some embodiments, R⁴ is selected from those depicted in Table 1.

As defined above and described herein, R⁵ is hydrogen, deuterium, an optionally substitute C₁₋₄ aliphatic, or —CN.

In some embodiments, R⁵ is hydrogen. In some embodiments, R⁵ is deuterium. In some embodiments, R⁵ is an optionally substituted C₁₋₄ aliphatic. In some embodiments, R⁵ is —CN.

In some embodiments, R⁵ is selected from those depicted in Table 1.

As defined above and described herein, each R⁶ is independently an optionally substituted group selected from C₁₋₆ aliphatic, phenyl, a 4-7 membered saturated or partially unsaturated heterocyclic ring having 1-3 heteroatoms independently selected from boron, nitrogen, oxygen, silicon, and sulfur, and a 5-6 membered heteroaryl ring having 1-4 heteroatoms independently selected from boron, nitrogen, oxygen, silicon, and sulfur.

In some embodiments, R⁶ is an optionally substituted C₁₋₆ aliphatic. In some embodiments, R⁶ is an optionally substituted phenyl. In some embodiments, R⁶ is an optionally substituted 4-7 membered saturated or partially unsaturated heterocyclic ring having 1-3 heteroatoms independently selected from boron, nitrogen, oxygen, silicon, and sulfur. In some embodiments, R⁶ is an optionally substituted 5-6 membered heteroaryl ring having 1-4 heteroatoms independently selected from boron, nitrogen, oxygen, silicon, and sulfur.

In some embodiments, R⁶ is selected from those depicted in Table 1.

As defined generally above, each R⁷ is independently hydrogen, deuterium, halogen, —CN, —OR, —SR, —S(O)R, —S(O)₂R, —N(R)₂, —P(O)(R)₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic, or R¹ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, or sulfur, or two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, or sulfur, or two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, or sulfur, or a 7-13 membered saturated, partially unsaturated, bridged heterocyclic ring, or a spiro heterocyclic ring having 1-3 heteroatoms, independently selected from boron, nitrogen, oxygen, silicon, or sulfur.

In some embodiments, R⁷ is hydrogen. In some embodiments, R⁷ is deuterium. In some embodiments, R⁷ is halogen. In some embodiments, R⁷ is —CN. In some embodiments, R⁷ is —OR. In some embodiments, R⁷ is —SR. In some embodiments, R⁷ is —S(O)R. In some embodiments, R⁷ is —S(O)₂R. In some embodiments, R⁷ is —NR₂. In some embodiments, R⁷ is —Si(R)₃. In some embodiments, R⁷ is —P(O)(R)₂. In some embodiments, R⁷ is —P(O)(OR)₂. In some embodiments, R⁷ is —P(O)(NR₂)OR. In some embodiments, R⁷ is —P(O)(NR₂)₂. In some embodiments, R⁷ is —Si(OH)R₂. In some embodiments, R⁷ is —Si(OH)₂R. In some embodiments, R⁷ is an optionally substituted C₁₋₄ aliphatic. In some embodiments, R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, or sulfur. In some embodiments, two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, or sulfur. In some embodiments, two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, or sulfur. In some embodiments, two R⁷ groups on adjacent carbon atoms are optionally taken together with their intervening atoms to form a 7-13 membered saturated, partially unsaturated, bridged heterocyclic ring, or a spiro heterocyclic ring having 1-3 heteroatoms, independently selected from boron, nitrogen, oxygen, silicon, or sulfur.

In some embodiments, R⁷ is selected from hydrogen, halogen, —CN, —OR, —NR₂, or C₁₋₄ alkyl. In some embodiments, R⁷ is selected from hydrogen, halogen, —CN, or C₁₋₄ alkyl. In some embodiments, R⁷ is fluoro. In some embodiments, two R⁷ groups on the same carbon are optionally taken together with their intervening atoms to form a 3- or 4-membered spiro fused ring.

In some embodiments, R⁷ is selected from those depicted in Table 1 below.

As defined above and described herein, Ring A is a bi- or tricyclic ring selected from

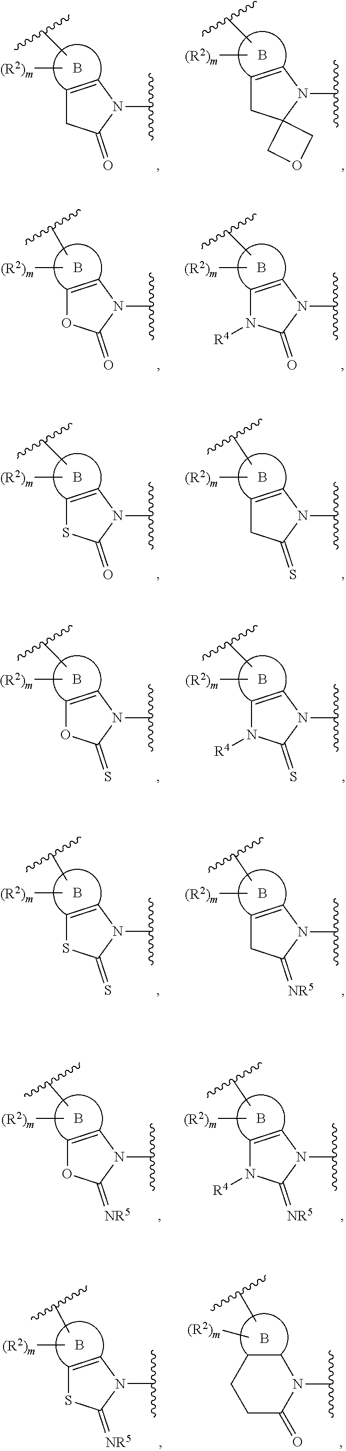

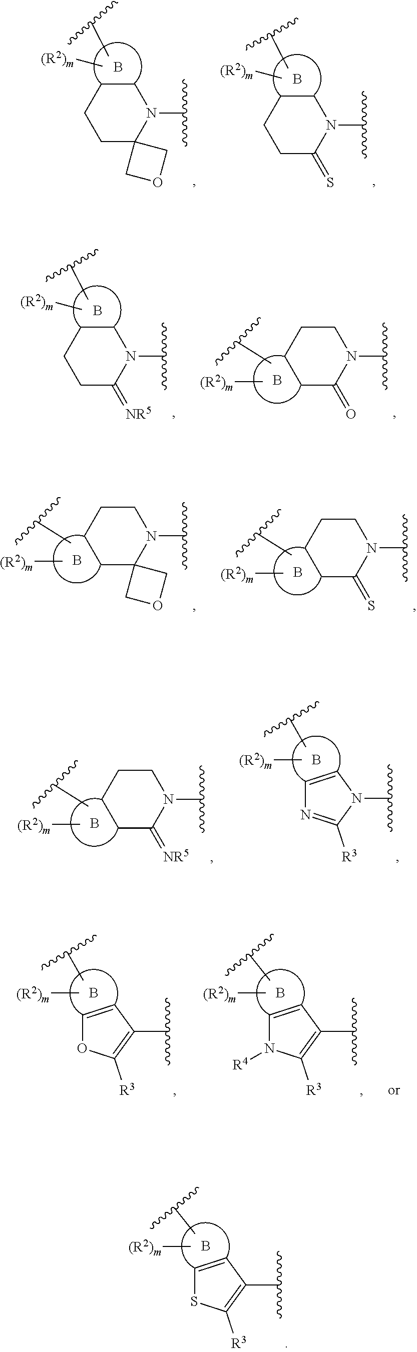

In some embodiments, Ring A is

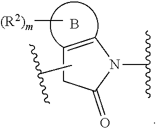

In some embodiments, Ring A is

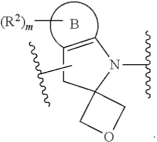

In some embodiments, Ring A is

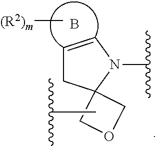

In some embodiments, Ring A is

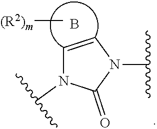

In some embodiments, Ring A is

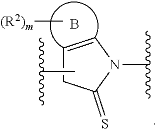

In some embodiments, Ring A is

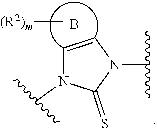

In some embodiments, Ring A is

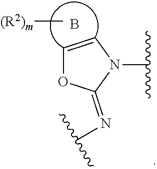

In some embodiments, Ring A is

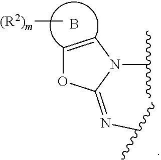

In some embodiments, Ring A is

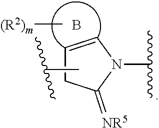

In some embodiments, Ring A is

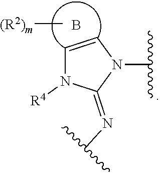

In some embodiments, Ring A is

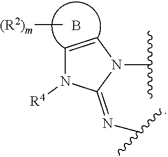

In some embodiments, Ring A is

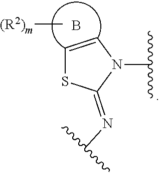

In some embodiments, Ring A is

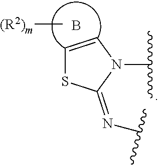

In some embodiments, Ring A is

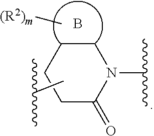

In some embodiments, Ring A is

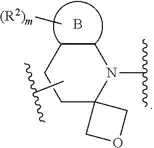

In some embodiments, Ring A is

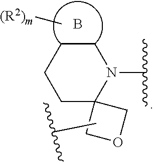

In some embodiments, Ring A is

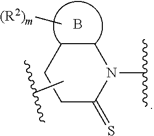

In some embodiments, Ring A is

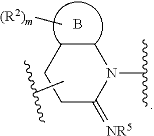

In some embodiments, Ring A is

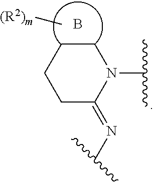

In some embodiments, Ring A is

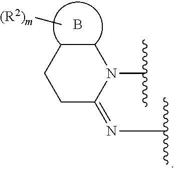

In some embodiments, Ring A is

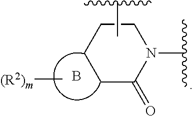

In some embodiments, Ring A is

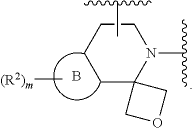

In some embodiments, Ring A is

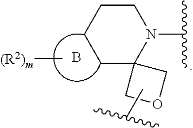

In some embodiments, Ring A is

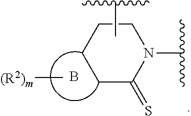

In some embodiments, Ring A is

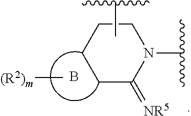

In some embodiments, Ring A is

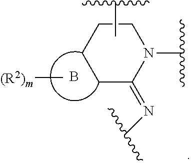

In some embodiments, Ring A is

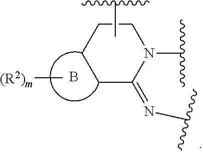

In some embodiments, Ring A is

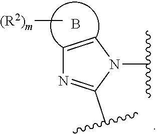

In some embodiments, Ring A is

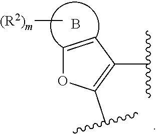

In some embodiments, Ring A is

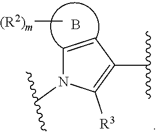

In some embodiments, Ring A is

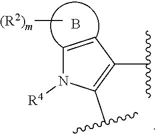

In some embodiments, Ring A is

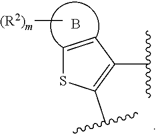

In some embodiments, Ring A is selected from those depicted in Table 1 below.

As defined above and described herein, Ring B is a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;

In some embodiments, Ring B is a fused 6-membered aryl. In some embodiments, Ring B is a fused 6-membered heteroaryl containing 1-4 heteroatoms independently selected from nitrogen, oxygen, or sulfur. In some embodiments, Ring B is a fused 5 to 7-membered saturated or partially unsaturated carbocyclyl. In some embodiments, Ring B is fused 5 to 7-membered saturated or partially saturated heterocyclyl with 1-3 heteroatoms independently selected from boron, nitrogen, oxygen, silicon, or sulfur. In some embodiments, Ring B is fused 5-membered heteroaryl with 1-4 heteroatoms independently selected from boron, nitrogen, oxygen, silicon, or sulfur.

In some embodiments, Ring B is

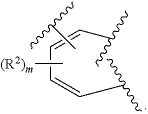

In some embodiments, Ring B is

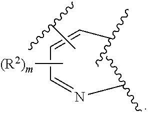

In some embodiments, Ring B is

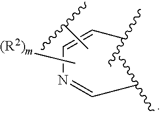

In some embodiments, Ring B is selected from those depicted in Table 1 below.

As defined above and described herein, Ring C is a mono- or bicyclic ring selected from

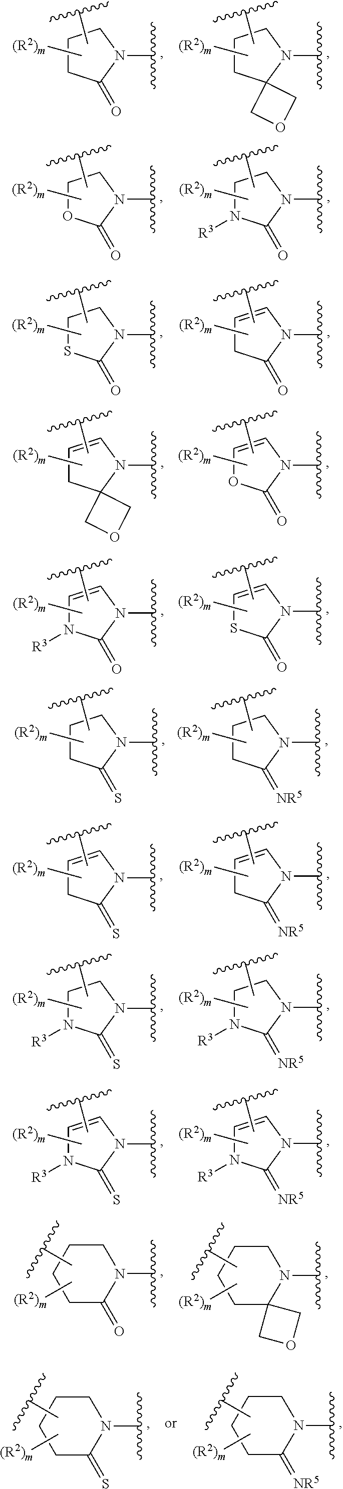

In some embodiments, Ring C is

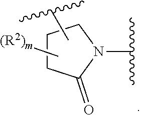

In some embodiments, Ring C is

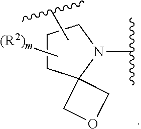

In some embodiments, Ring C is

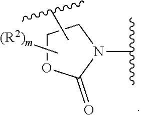

In some embodiments, Ring C is

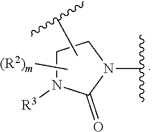

In some embodiments, Ring C is

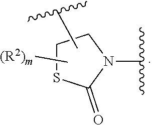

In some embodiments, Ring C is

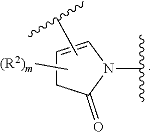

In some embodiments, Ring C is

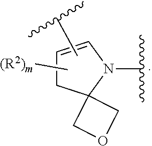

In some embodiments, Ring C is

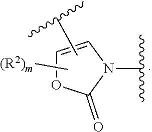

In some embodiments, Ring C is

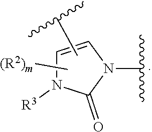

In some embodiments, Ring C is

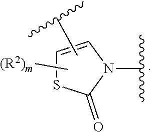

In some embodiments, Ring C is

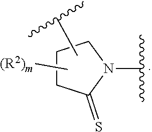

In some embodiments, Ring C is

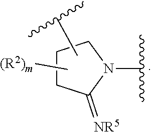

In some embodiments, Ring C is

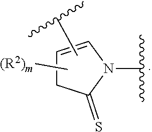

In some embodiments, Ring C is

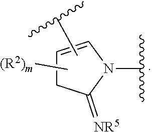

In some embodiments, Ring C is

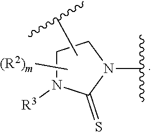

In some embodiments, Ring C is

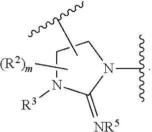

In some embodiments, Ring C is

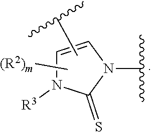

In some embodiments, Ring C is

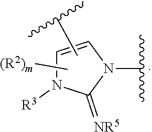

In some embodiments, Ring C is

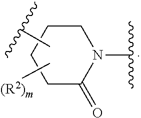

In some embodiments, Ring C is

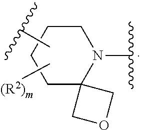

In some embodiments, Ring C is

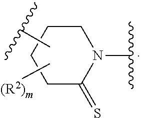

In some embodiments, Ring C is

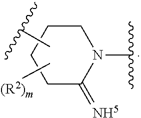

In some embodiments, Ring C is

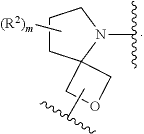

In some embodiments, Ring C is

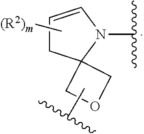

In some embodiments, Ring C is

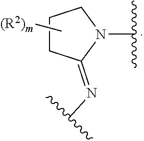

In some embodiments, Ring C is

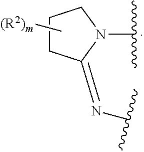

In some embodiments, Ring C is

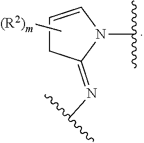

In some embodiments, Ring C is

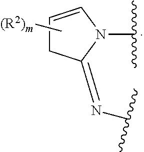

In some embodiments, Ring C is

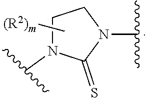

In some embodiments, Ring C is

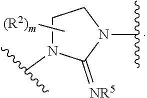

In some embodiments, Ring C is

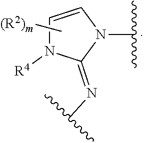

In some embodiments, Ring C is

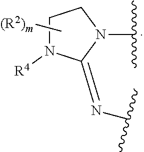

In some embodiments, Ring C is

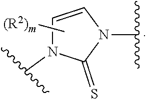

In some embodiments, Ring C is

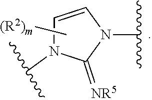

In some embodiments, Ring C is

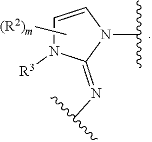

In some embodiments, Ring C is

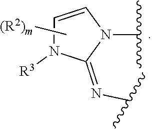

In some embodiments, Ring C is

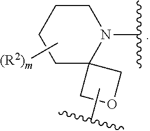

In some embodiments, Ring C is

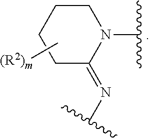

In some embodiments, Ring C is

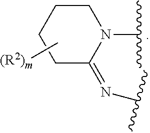

In some embodiments, Ring C is a mono- or bicyclic ring selected from

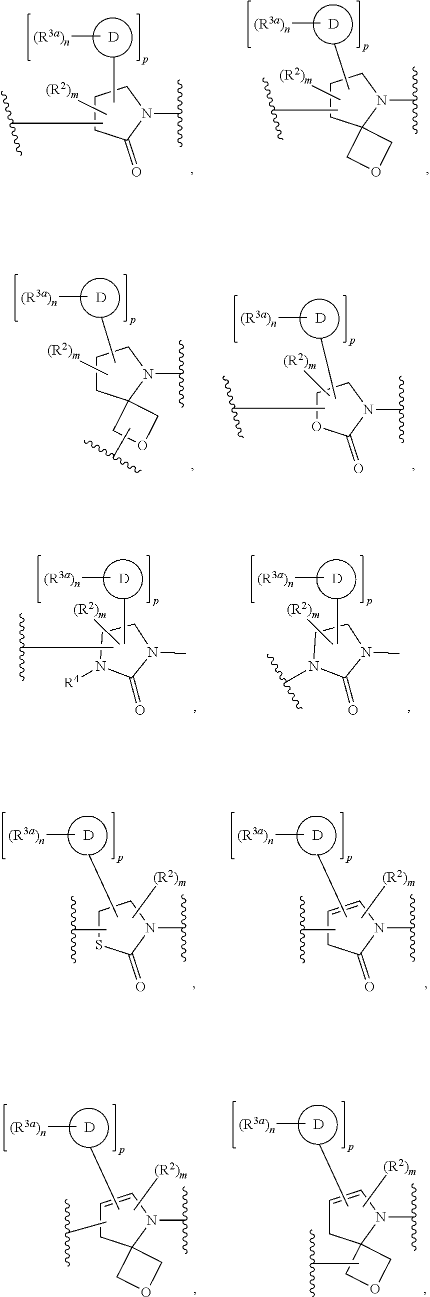

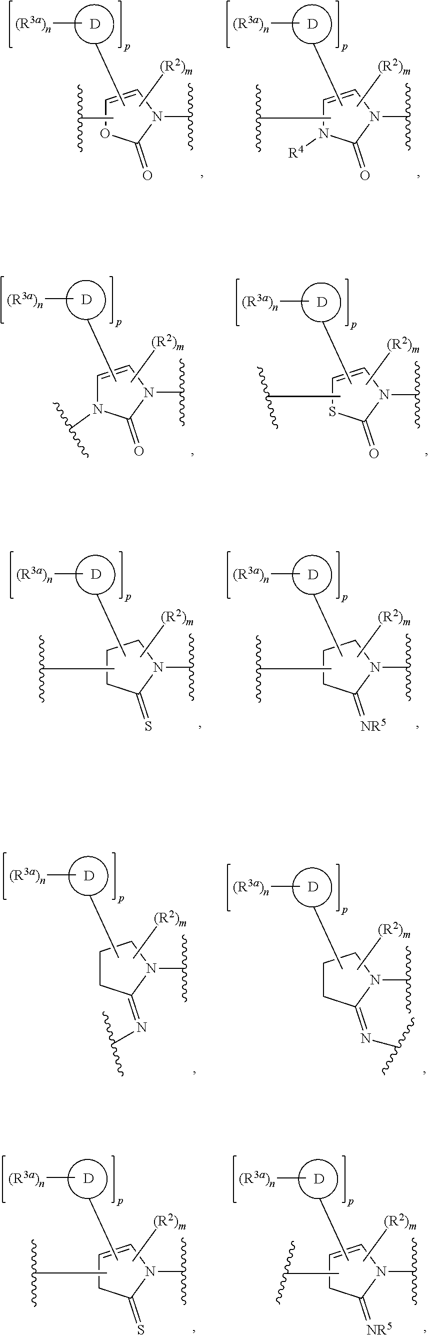

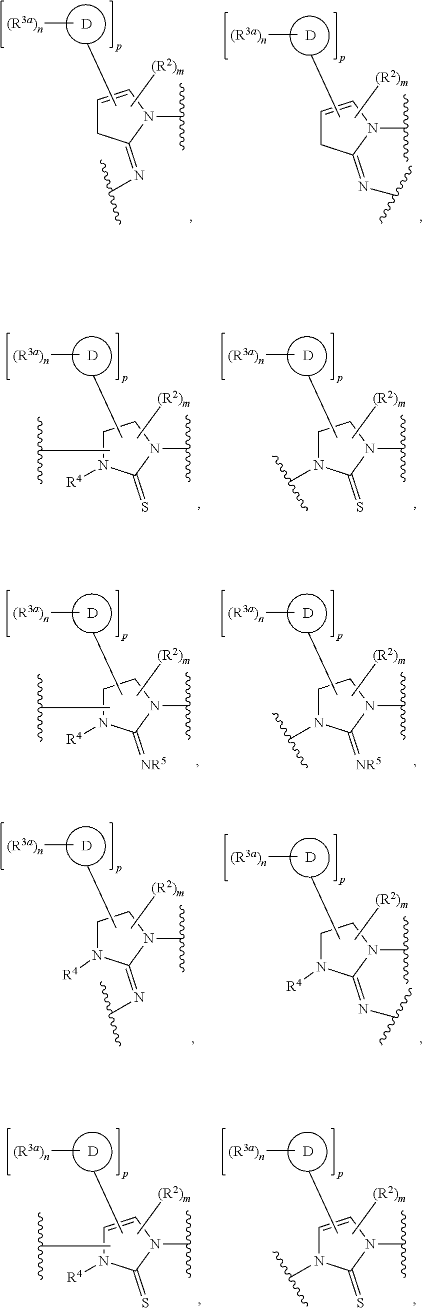

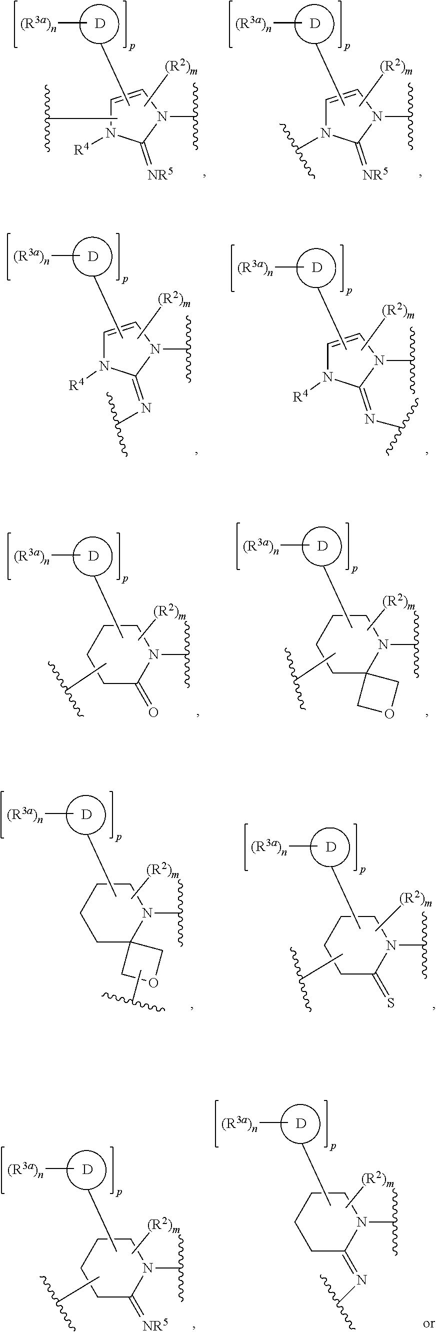

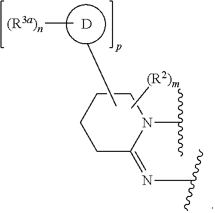

In some embodiments, Ring C is selected from those depicted in Table 1 below.

As defined above and described herein, Ring D is a ring selected from 6 to 10-membered aryl or heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;

In some embodiments, Ring D is a 6 to 10-membered aryl. In some embodiments, Ring D is a 6 to 10-membered heteroaryl containing 1-4 heteroatoms independently selected from nitrogen, oxygen, or sulfur. In some embodiments, Ring D is a 5 to 7-membered saturated or partially unsaturated carbocyclyl. In some embodiments, Ring D is 5 to 7-membered saturated or partially saturated heterocyclyl with 1-3 heteroatoms independently selected from boron, nitrogen, oxygen, silicon, or sulfur. In some embodiments, Ring D is 5-membered heteroaryl with 1-4 heteroatoms independently selected from boron, nitrogen, oxygen, silicon, or sulfur.

In some embodiments, Ring D phenyl. In some embodiments, Ring D is pyridyl. In some embodiments, Ring D naphthyl. In some embodiments, Ring D is isoquinolinyl. In some embodiments, Ring D is imidazopyridyl (e.g., imidazo[1,2-a]pyridyl). In some embodiments, Ring D is indazolyl. In some embodiments, Ring D is benzoisoxazolyl (e.g., benzo[d]isoxazolyl).

In some embodiments, Ring D is Ring A.

In some embodiments, Ring D is selected from those depicted in Table 1 below.

As defined above and described herein, each of Ring E, Ring F, and Ring G is independently a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur, wherein Ring E, Ring F, and Ring G is independently and optionally substituted with 1-2 oxo groups.

In some embodiments, each of Ring E, Ring F, and Ring G is independently a fused ring selected from 6-membered aryl. In some embodiments, each of Ring E, Ring F, and Ring G is independently a fused ring selected from 6-membered heteroaryl containing 1-4 heteroatoms independently selected from nitrogen, oxygen, or sulfur. In some embodiments, each of Ring E, Ring F, and Ring G is independently a fused ring selected from a 5 to 7-membered saturated or partially unsaturated carbocyclyl. In some embodiments, each of Ring E, Ring F, and Ring G is independently a fused ring selected from a 5 to 7-membered saturated or partially unsaturated heterocyclyl with 1-3 heteroatoms independently selected from boron, nitrogen, oxygen, silicon, or sulfur. In some embodiments, each of Ring E, Ring F, and Ring G is independently a fused ring selected from a 5-membered heteroaryl with 1-4 heteroatoms independently selected from nitrogen, oxygen or sulfur. In some embodiments, Ring E, Ring F, and Ring G is independently and optionally substituted with 1-2 oxo groups.

In some embodiments, Ring F is

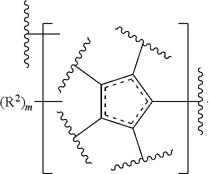

In some embodiments, each of Ring E and Ring G is independently

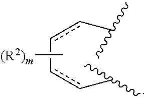

In some embodiments, each of Ring E and Ring G is independently

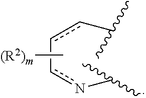

In some embodiments, each of Ring E and Ring G is independently

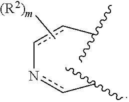

In some embodiments, each of Ring E and Ring G is independently

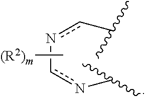

In some embodiments, each of Ring E and Ring G is independently

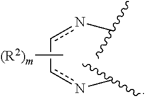

In some embodiments, Ring E, Ring F, and Ring G is

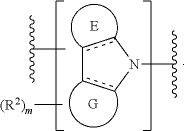

In some embodiments, Ring E, Ring F, and Ring G is

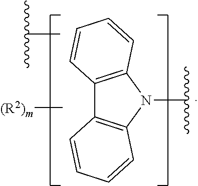

In some embodiments, Ring E, Ring F, and Ring G is

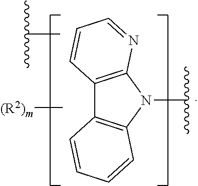

In some embodiments, Ring E, Ring F, and Ring G is selected from those depicted in Table 1, below.

As defined above and described herein, Ring H is a ring selected from a 7-9 membered saturated or partially unsaturated carbocyclyl or heterocyclyl ring with 1-3 heteroatoms independently selected from boron, nitrogen, oxygen, silicon, or sulfur, wherein Ring E is optionally further substituted with 1-2 oxo groups.

In some embodiments, Ring H is a ring selected from a 7-9 membered saturated or partially unsaturated carbocyclyl or heterocyclyl ring with 1-3 heteroatoms independently selected from boron, nitrogen, oxygen, silicon, or sulfur, wherein Ring H is optionally further substituted with 1-2 oxo groups.

As defined above and described herein, each of Ring I and Ring J is independently a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur

In some embodiments, each of Ring I and Ring J is independently a 6-membered aryl. In some embodiments, each of Ring I and Ring J is independently a 6-membered heteroaryl containing 1-4 heteroatoms independently selected from nitrogen, oxygen, or sulfur. In some embodiments, each of Ring I and Ring J is independently a 5 to 7-membered saturated or partially unsaturated carbocyclyl. In some embodiments, each of Ring I and Ring J is independently a 5 to 7-membered saturated or partially unsaturated heterocyclyl with 1-3 heteroatoms independently selected from boron, nitrogen, oxygen, silicon, or sulfur. In some embodiments, each of Ring I and Ring J is independently a 5-membered heteroaryl with 1-3 heteroatoms independently selected from nitrogen, oxygen or sulfur.

In some embodiments, Ring I and Ring J is selected from those depicted in Table 1, below.

As defined above and described herein, Ring K is a fused ring selected from a 5-12 membered saturated or partially unsaturated carbocyclyl or heterocyclyl with 1-3 heteroatoms independently selected from boron, nitrogen, oxygen, silicon, or sulfur, wherein Ring H is optionally further substituted with 1-2 oxo groups.

In some embodiments, Ring K is a fused ring selected from a 5-12 membered saturated or partially unsaturated carbocyclyl. In some embodiments, Ring K is a 5-12 membered saturated or partially unsaturated heterocyclyl ring with 1-3 heteroatoms independently selected from boron, nitrogen, oxygen, silicon, or sulfur. In some embodiments, Ring K is a fused 5-6 membered saturated or partially unsaturated heterocyclyl ring with 1-3 heteroatoms independently selected from boron, nitrogen, oxygen, silicon, or sulfur. In some embodiments, Ring K is optionally further substituted with 1-2 oxo groups.

In some embodiments, Ring I, Ring J, and Ring K is

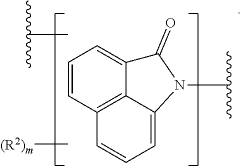

In some embodiments, Ring K is selected from those depicted in Table 1 below.

As defined above and described herein, Ring M is selected from

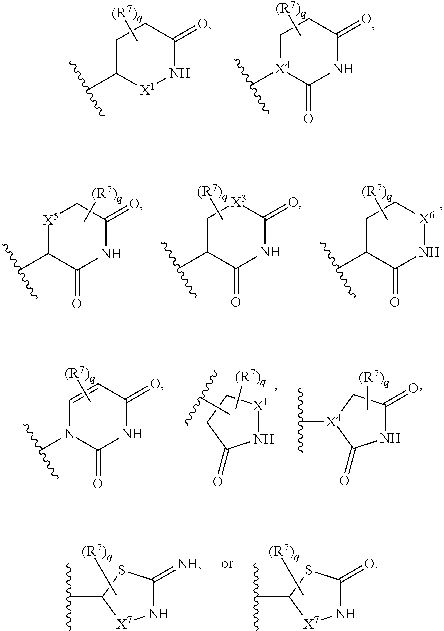

In some embodiments, Ring M is

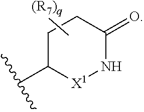

In some embodiments, Ring M is

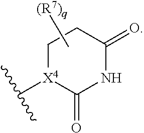

In some embodiments, Ring M is

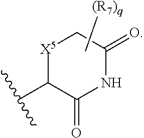

In some embodiments, Ring M is

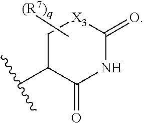

In some embodiments, Ring M is

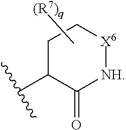

In some embodiments, Ring M is

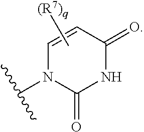

In some embodiments, Ring M is

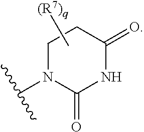

In some embodiments, Ring M is

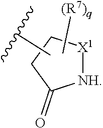

In some embodiments, Ring M is

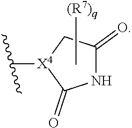

In some embodiments, Ring M is

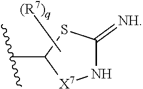

In some embodiments, Ring M is

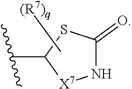

In some embodiments, Ring M is selected from those depicted in Table 1 below.

As defined above and described here, L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;

In some embodiments, L¹ is a covalent bond. In some embodiments, L¹ is a C₁₋₃ aliphatic. In some embodiments, L¹ is —CH₂—. In some embodiments, L¹ is —C(D)(H)—. In some embodiments, L¹ is —C(D)₂-. In some embodiments, L¹ is —CH₂CH₂—. In some embodiments, L¹ is —NR—. In some embodiments, L¹ is —NH—. In some embodiments, L¹ is —NMe-. In some embodiments, L¹ is -NEt-. In some embodiments, L¹ is —CH₂NR—. In some embodiments, L¹ is or —O—. In some embodiments, L¹ is —CH₂O—. In some embodiments, L¹ is —S—. In some embodiments, L¹ is —OC(O)—. In some embodiments, L¹ is —C(O)O—. In some embodiments, L¹ is —C(O)—. In some embodiments, L¹ is —S(O)—. In some embodiments, L¹ is —S(O)₂—. In some embodiments, L¹ is —NRS(O)₂—. In some embodiments, L¹ is —S(O)₂NR—. In some embodiments, L¹ is —NRC(O)—. In some embodiments, L¹ is —C(O)NR—.

In some embodiments, Ring L¹ is selected from those depicted in Table 1 below.

As defined above and described herein [symbol] is a single or double bond.

In some embodiments, [symbol] is a single bond. In some embodiments, [symbol] is a double bond.

In some embodiments, [symbol] is selected from those depicted in Table 1 below.

[symbol]

As defined above and described herein, m is 0, 1, 2, 3, 4, 5, 6, 7, 8, 9, 10, 11, 12, 13, 14, 15, or 16.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16.

In some embodiments, m is selected from those depicted in Table 1 below.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In some embodiments, n is selected from those depicted in Table 1 below.

As defined above and described herein, p is 0 or 1.

In some embodiments, p is 0. In some embodiments, p is 1.

In some embodiments, p is selected from those depicted in Table 1 below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1 below.

In some embodiments, LBM is

In some embodiments, LBM is

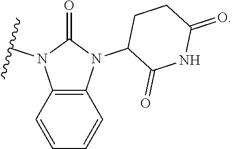 [SMILES: *n1c(=O)n(C2CCC(=O)NC2=O)c2ccccc21]

In some embodiments, LBM is

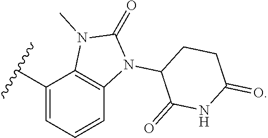 [SMILES: *c1cccc2c1n(C)c(=O)n2C1CCC(=O)NC1=O]

In some embodiments, LBM is

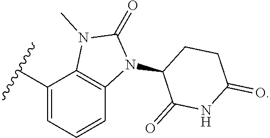 [SMILES: *c1cccc2c1n(C)c(=O)n2[C@H]1CCC(=O)NC1=O]

In some embodiments, LBM is

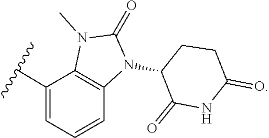 [SMILES: *c1cccc2c1n(C)c(=O)n2[C@@H]1CCC(=O)NC1=O]

In some embodiments, LBM is

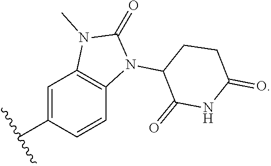 [SMILES: *c1ccc2c(c1)n(C)c(=O)n2C1CCC(=O)NC1=O]

In some embodiments, LBM is

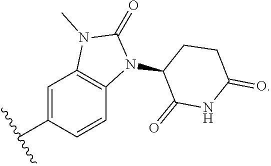 [SMILES: *c1ccc2c(c1)n(C)c(=O)n2[C@H]1CCC(=O)NC1=O]

In some embodiments, LBM is

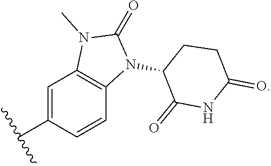 [SMILES: *c1ccc2c(c1)n(C)c(=O)n2[C@@H]1CCC(=O)NC1=O]

In some embodiments, LBM is

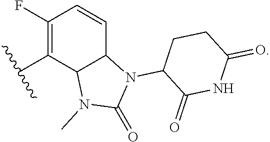 [SMILES: *C1=C(F)C=CC2C1N(C)C(=O)N2C1CCC(=O)NC1=O]

In some embodiments, LBM is

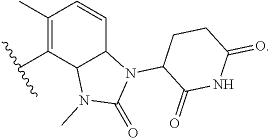 [SMILES: *C1=C(C)C=CC2C1N(C)C(=O)N2C1CCC(=O)NC1=O]

In some embodiments, LBM is

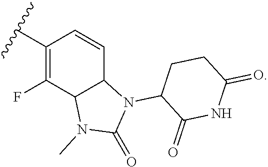 [SMILES: *C1=C(F)C2C(C=C1)N(C1CCC(=O)NC1=O)C(=O)N2C]

In some embodiments, LBM is

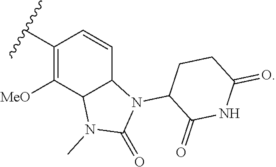 [SMILES: *C1=C(OC)C2C(C=C1)N(C1CCC(=O)NC1=O)C(=O)N2C]

In some embodiments, LBM is

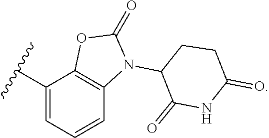 [SMILES: *c1cccc2c1oc(=O)n2C1CCC(=O)NC1=O]

In some embodiments, LBM is

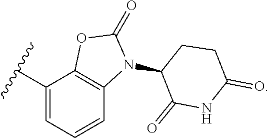 [SMILES: *c1cccc2c1oc(=O)n2[C@H]1CCC(=O)NC1=O]

In some embodiments, LBM is

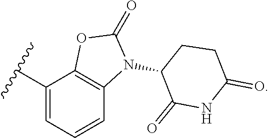 [SMILES: *c1cccc2c1oc(=O)n2[C@@H]1CCC(=O)NC1=O]

In some embodiments, LBM is

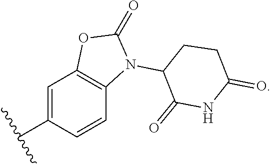 [SMILES: *c1ccc2c(c1)oc(=O)n2C1CCC(=O)NC1=O]

In some embodiments, LBM is

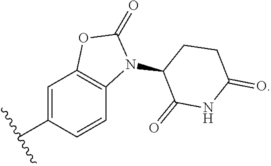 [SMILES: *c1ccc2c(c1)oc(=O)n2[C@H]1CCC(=O)NC1=O]

In some embodiments, LBM is

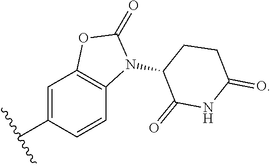 [SMILES: *c1ccc2c(c1)oc(=O)n2[C@@H]1CCC(=O)NC1=O]

In some embodiments, LBM is

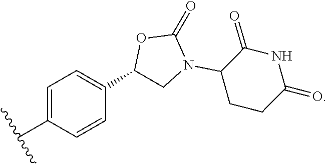 [SMILES: *c1ccc([C@H]2CN(C3CCC(=O)NC3=O)C(=O)O2)cc1]

In some embodiments, LBM is

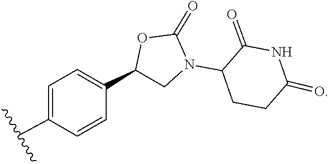 [SMILES: *c1ccc([C@@H]2CN(C3CCC(=O)NC3=O)C(=O)O2)cc1]

In some embodiments, LBM is

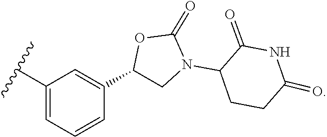 [SMILES: *c1cccc([C@H]2CN(C3CCC(=O)NC3=O)C(=O)O2)c1]

In some embodiments, LBM is

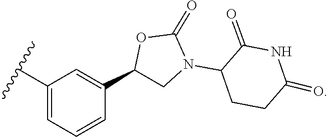 [SMILES: *c1cccc([C@@H]2CN(C3CCC(=O)NC3=O)C(=O)O2)c1]

In some embodiments, LBM is

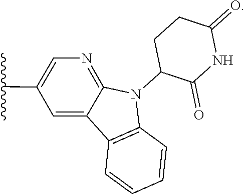 [SMILES: *c1cnc2c(c1)c1ccccc1n2C1CCC(=O)NC1=O]

In some embodiments, LBM is

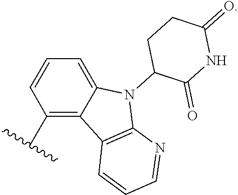 [SMILES: *c1cccc2c1c1cccnc1n2C1CCC(=O)NC1=O]

In some embodiments, LBM is

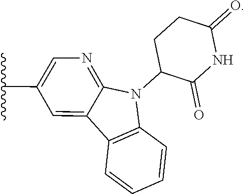 [SMILES: *c1cnc2c(c1)c1ccccc1n2C1CCC(=O)NC1=O]

In some embodiments, LBM is

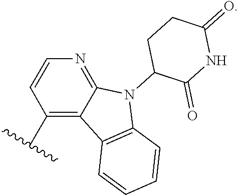 [SMILES: *c1ccnc2c1c1ccccc1n2C1CCC(=O)NC1=O]

In some embodiments, LBM is

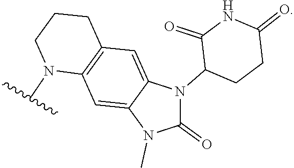 [SMILES: *N1CCCc2cc3c(cc21)n(C)c(=O)n3C1CCC(=O)NC1=O]

In some embodiments, LBM is

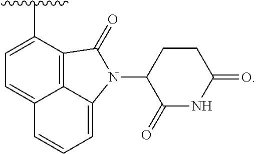 [SMILES: *c1ccc2cccc3c2c1C(=O)N3C1CCC(=O)NC1=O]

In some embodiments, LBM is

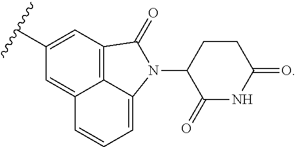 [SMILES: *c1cc2c3c(cccc3c1)N(C1CCC(=O)NC1=O)C2=O]

In some embodiments, LBM is

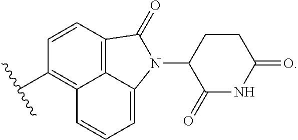 [SMILES: *c1ccc2c3c(cccc13)N(C1CCC(=O)NC1=O)C2=O]

In some embodiments, LBM is
In some embodiments, LBM is

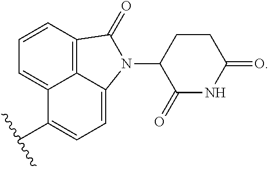 [SMILES: *c1ccc2c3c(cccc13)C(=O)N2C1CCC(=O)NC1=O]

In some embodiments, LBM is

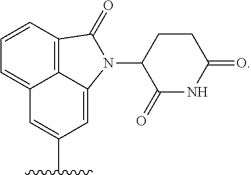 [SMILES: *c1cc2c3c(cccc3c1)C(=O)N2C1CCC(=O)NC1=O]

In some embodiments, LBM is

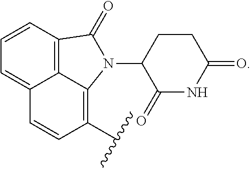 [SMILES: *c1ccc2cccc3c2c1N(C1CCC(=O)NC1=O)C3=O]

In some embodiments, LBM is

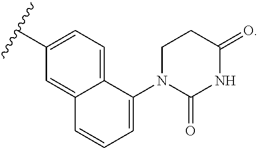 [SMILES: *c1ccc2c(N3CCC(=O)NC3=O)cccc2c1]

In some embodiments, LBM is

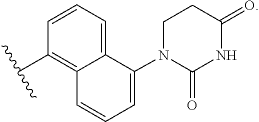 [SMILES: *c1cccc2c(N3CCC(=O)NC3=O)cccc12]

In some embodiments, LBM is

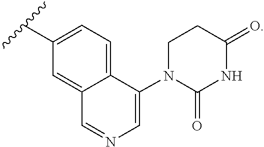 [SMILES: *c1ccc2c(N3CCC(=O)NC3=O)cncc2c1]

In some embodiments, LBM is

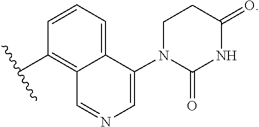 [SMILES: *c1cccc2c(N3CCC(=O)NC3=O)cncc12]

In some embodiments, LBM is

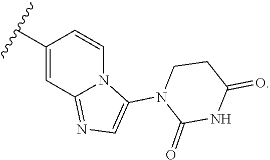 [SMILES: *c1ccn2c(N3CCC(=O)NC3=O)cnc2c1]

In some embodiments, LBM is

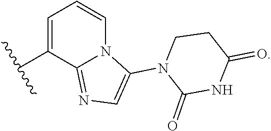 [SMILES: *c1cccn2c(N3CCC(=O)NC3=O)cnc12]

In some embodiments, LBM is

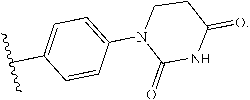 [SMILES: *c1ccc(N2CCC(=O)NC2=O)cc1]

In some embodiments, LBM is

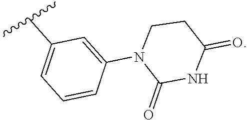 [SMILES: *c1cccc(N2CCC(=O)NC2=O)c1]

In some embodiments, LBM is

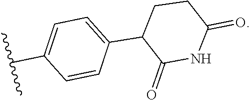 [SMILES: *c1ccc(C2CCC(=O)NC2=O)cc1]

In some embodiments, LBM is

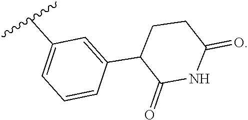 [SMILES: *c1cccc(C2CCC(=O)NC2=O)c1]

In some embodiments, LBM is

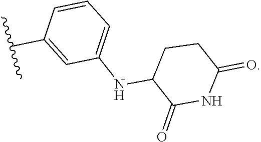 [SMILES: *c1cccc(NC2CCC(=O)NC2=O)c1]

In some embodiments, LBM is

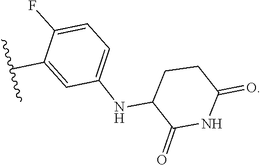 [SMILES: *c1cc(NC2CCC(=O)NC2=O)ccc1F]

In some embodiments, LBM is

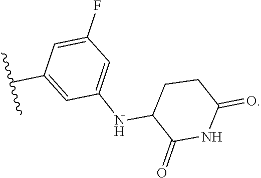 [SMILES: *c1cc(F)cc(NC2CCC(=O)NC2=O)c1]

In some embodiments, LBM is

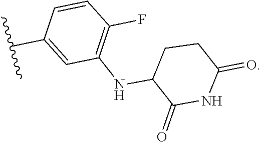 [SMILES: *c1ccc(F)c(NC2CCC(=O)NC2=O)c1]

In some embodiments, LBM is

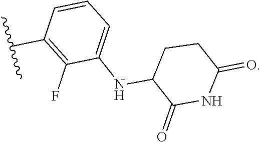 [SMILES: *c1cccc(NC2CCC(=O)NC2=O)c1F]

In some embodiments, LBM is

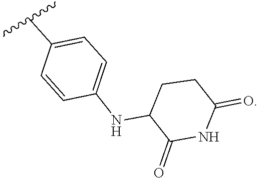 [SMILES: *c1ccc(NC2CCC(=O)NC2=O)cc1]

In some embodiments, LBM is

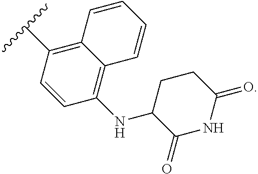 [SMILES: *c1ccc(NC2CCC(=O)NC2=O)c2ccccc12]

In some embodiments, LBM is

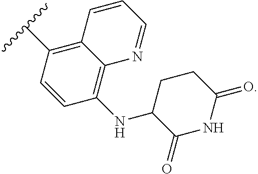 [SMILES: *c1ccc(NC2CCC(=O)NC2=O)c2ncccc12]

In some embodiments, LBM is

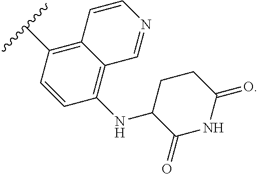 [SMILES: *c1ccc(NC2CCC(=O)NC2=O)c2cnccc12]

In some embodiments, LBM is

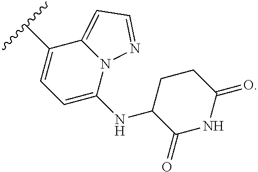 [SMILES: *c1ccc(NC2CCC(=O)NC2=O)n2nccc12]

In some embodiments, LBM is

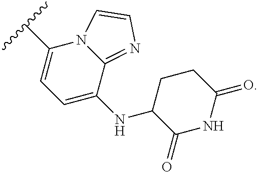 [SMILES: *c1ccc(NC2CCC(=O)NC2=O)c2nccn12]

In some embodiments, LBM is

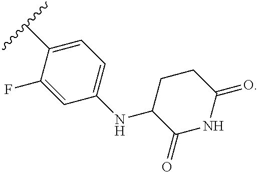 [SMILES: *c1ccc(NC2CCC(=O)NC2=O)cc1F]

In some embodiments, LBM is

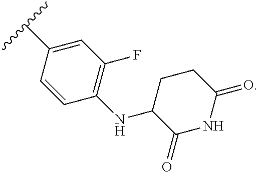 [SMILES: *c1ccc(NC2CCC(=O)NC2=O)c(F)c1]

In some embodiments, LBM is

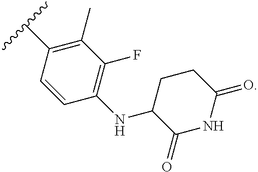 [SMILES: *c1ccc(NC2CCC(=O)NC2=O)c(F)c1C]

In some embodiments, LBM is

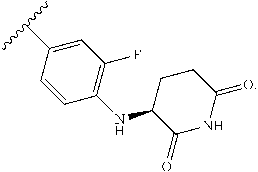 [SMILES: *c1ccc(N[C@H]2CCC(=O)NC2=O)c(F)c1]

In some embodiments, LBM is

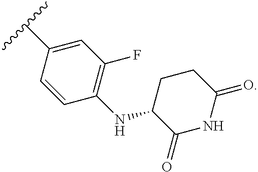 [SMILES: *c1ccc(N[C@@H]2CCC(=O)NC2=O)c(F)c1]

In some embodiments, LBM is

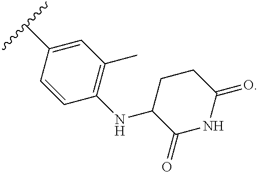 [SMILES: *c1ccc(NC2CCC(=O)NC2=O)c(C)c1]

In some embodiments, LBM is

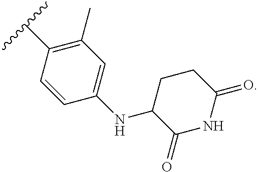 [SMILES: *c1ccc(NC2CCC(=O)NC2=O)cc1C]

In some embodiments, LBM is

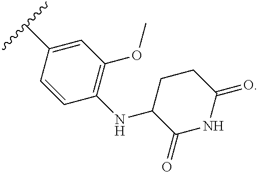 [SMILES: *c1ccc(NC2CCC(=O)NC2=O)c(OC)c1]

In some embodiments, LBM is

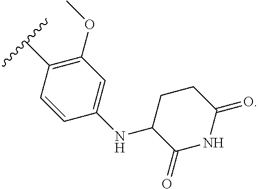 [SMILES: *c1ccc(NC2CCC(=O)NC2=O)cc1OC]

In some embodiments, LBM is

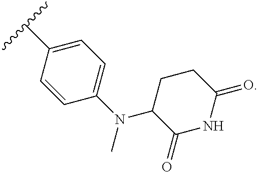 [SMILES: *c1ccc(N(C)C2CCC(=O)NC2=O)cc1]

In some embodiments, LBM is

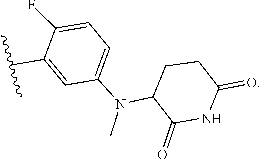 [SMILES: *c1cc(N(C)C2CCC(=O)NC2=O)ccc1F]

In some embodiments, LBM is

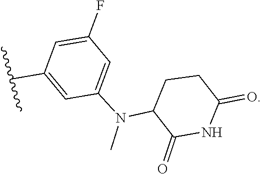 [SMILES: *c1cc(F)cc(N(C)C2CCC(=O)NC2=O)c1]

In some embodiments, LBM is

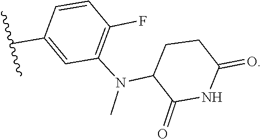 [SMILES: *c1ccc(F)c(N(C)C2CCC(=O)NC2=O)c1]

In some embodiments, LBM is

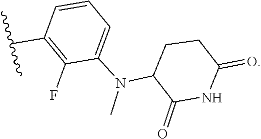 [SMILES: *c1cccc(N(C)C2CCC(=O)NC2=O)c1F]

In some embodiments, LBM is

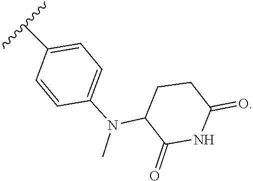 [SMILES: *c1ccc(N(C)C2CCC(=O)NC2=O)cc1]

In some embodiments, LBM is

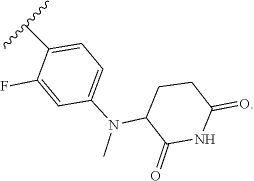 [SMILES: *c1ccc(N(C)C2CCC(=O)NC2=O)cc1F]

In some embodiments, LBM is

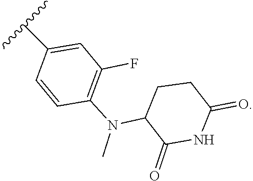 [SMILES: *c1ccc(N(C)C2CCC(=O)NC2=O)c(F)c1]

In some embodiments, LBM is

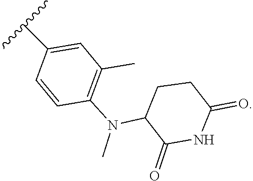 [SMILES: *c1ccc(N(C)C2CCC(=O)NC2=O)c(C)c1]

In some embodiments, LBM is

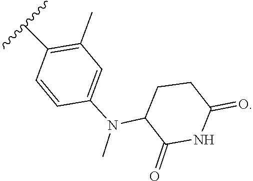 [SMILES: *c1ccc(N(C)C2CCC(=O)NC2=O)cc1C]

In some embodiments, LBM is

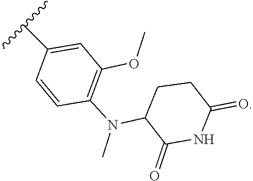 [SMILES: *c1ccc(N(C)C2CCC(=O)NC2=O)c(OC)c1]

In some embodiments, LBM is

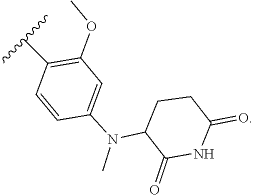 [SMILES: *c1ccc(N(C)C2CCC(=O)NC2=O)cc1OC]

In some embodiments, LBM is

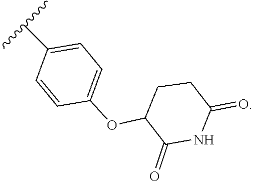 [SMILES: *c1ccc(OC2CCC(=O)NC2=O)cc1]

In some embodiments, LBM is

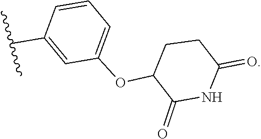 [SMILES: *c1cccc(OC2CCC(=O)NC2=O)c1]

In some embodiments, LBM is

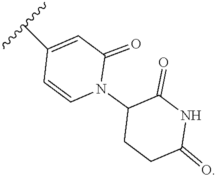 [SMILES: *c1ccn(C2CCC(=O)NC2=O)c(=O)c1]

In some embodiments, LBM is

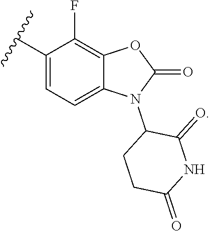 [SMILES: *c1ccc2c(oc(=O)n2C2CCC(=O)NC2=O)c1F]

In some embodiments, LBM is

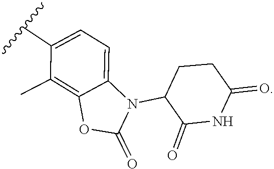 [SMILES: *c1ccc2c(oc(=O)n2C2CCC(=O)NC2=O)c1C]

In some embodiments, LBM is

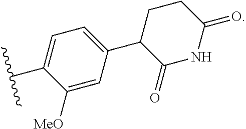 [SMILES: *c1ccc(C2CCC(=O)NC2=O)cc1OC]

In some embodiments, LBM is

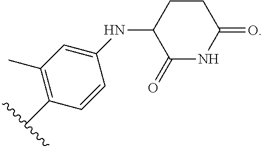 [SMILES: *c1ccc(NC2CCC(=O)NC2=O)cc1C]

In some embodiments, LBM is

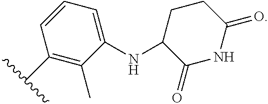 [SMILES: *c1cccc(NC2CCC(=O)NC2=O)c1C]

In some embodiments, LBM is

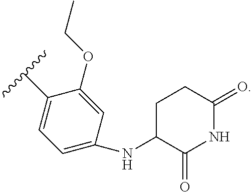 [SMILES: *c1ccc(NC2CCC(=O)NC2=O)cc1OCC]

In some embodiments, LBM is

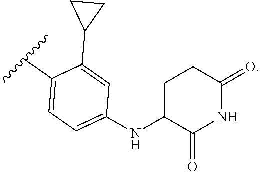 [SMILES: *c1ccc(NC2CCC(=O)NC2=O)cc1C1CC1]

In some embodiments, LBM is

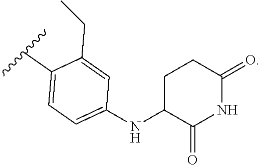 [SMILES: *c1ccc(NC2CCC(=O)NC2=O)cc1CC]

In some embodiments, LBM is

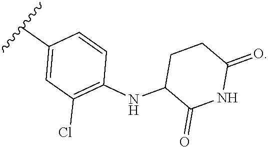 [SMILES: *c1ccc(NC2CCC(=O)NC2=O)c(Cl)c1]

In some embodiments, LBM is

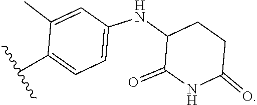 [SMILES: *c1ccc(NC2CCC(=O)NC2=O)cc1C]

In some embodiments, LBM is

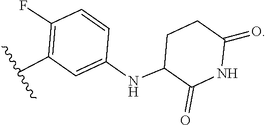 [SMILES: *c1cc(NC2CCC(=O)NC2=O)ccc1F]

In some embodiments, LBM is

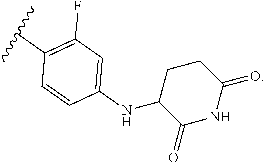 [SMILES: *c1ccc(NC2CCC(=O)NC2=O)cc1F]

In some embodiments, LBM is

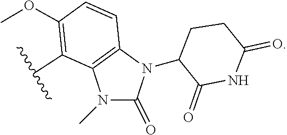 [SMILES: *c1c(OC)ccc2c1n(C)c(=O)n2C1CCC(=O)NC1=O]

In some embodiments, LBM is

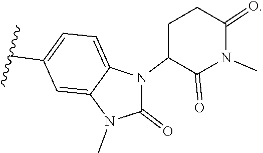 [SMILES: *c1ccc2c(c1)n(C)c(=O)n2C1CCC(=O)N(C)C1=O]

In some embodiments, LBM is

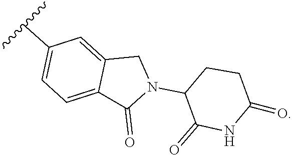 [SMILES: *c1ccc2c(c1)CN(C1CCC(=O)NC1=O)C2=O]

In some embodiments, LBM is

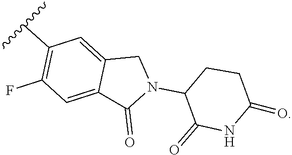 [SMILES: *c1cc2c(cc1F)C(=O)N(C1CCC(=O)NC1=O)C2]

In some embodiments, LBM is

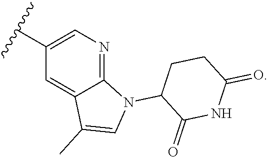 [SMILES: *c1cnc2c(c1)c(C)cn2C1CCC(=O)NC1=O]

In some embodiments, LBM is

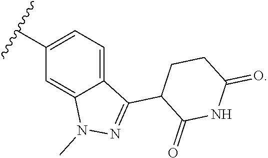 [SMILES: *c1ccc2c(C3CCC(=O)NC3=O)nn(C)c2c1]

In some embodiments, LBM is

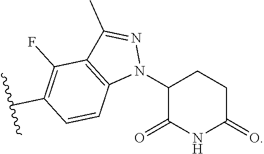 [SMILES: *c1ccc2c(c(C)nn2C2CCC(=O)NC2=O)c1F]

In some embodiments, LBM is

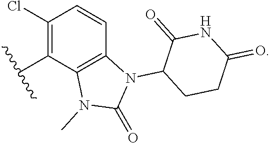 [SMILES: *c1c(Cl)ccc2c1n(C)c(=O)n2C1CCC(=O)NC1=O]

In some embodiments, LBM is

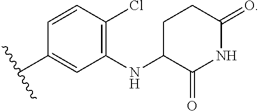 [SMILES: *c1ccc(Cl)c(NC2CCC(=O)NC2=O)c1]

In some embodiments, LBM is

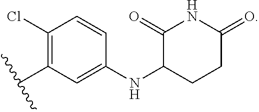 [SMILES: *c1cc(NC2CCC(=O)NC2=O)ccc1Cl]

In some embodiments, LBM is

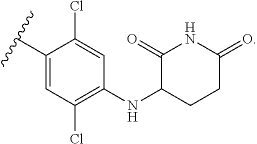 [SMILES: *c1cc(Cl)c(NC2CCC(=O)NC2=O)cc1Cl]

In some embodiments, LBM is

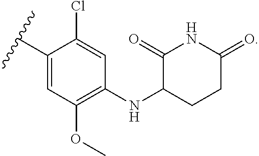 [SMILES: *c1cc(OC)c(NC2CCC(=O)NC2=O)cc1Cl]

In some embodiments, LBM is

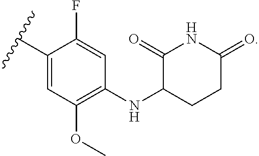 [SMILES: *c1cc(OC)c(NC2CCC(=O)NC2=O)cc1F]

In some embodiments, LBM is

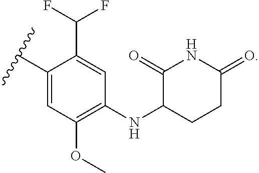 [SMILES: *c1cc(OC)c(NC2CCC(=O)NC2=O)cc1C(F)F]

In some embodiments, LBM is

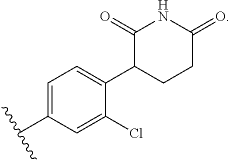 [SMILES: *c1ccc(C2CCC(=O)NC2=O)c(Cl)c1]

In some embodiments, LBM is

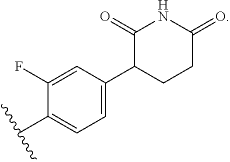 [SMILES: *c1ccc(C2CCC(=O)NC2=O)cc1F]

In some embodiments, LBM is

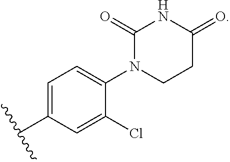 [SMILES: *c1ccc(N2CCC(=O)NC2=O)c(Cl)c1]

In some embodiments, LBM is

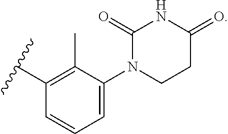 [SMILES: *c1cccc(N2CCC(=O)NC2=O)c1C]

In some embodiments, LBM is

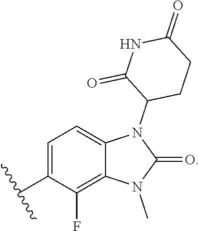 [SMILES: *c1ccc2c(c1F)n(C)c(=O)n2C1CCC(=O)NC1=O]

In some embodiments, LBM is

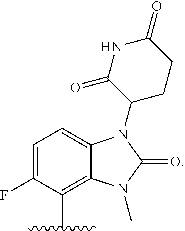 [SMILES: *c1c(F)ccc2c1n(C)c(=O)n2C1CCC(=O)NC1=O]

In some embodiments, LBM is

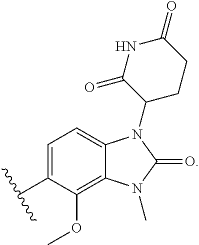 [SMILES: *c1ccc2c(c1OC)n(C)c(=O)n2C1CCC(=O)NC1=O]

In some embodiments, LBM is

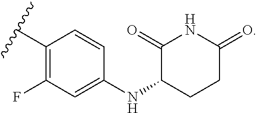 [SMILES: *c1ccc(N[C@H]2CCC(=O)NC2=O)cc1F]

In some embodiments, LBM is

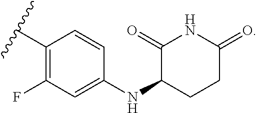 [SMILES: *c1ccc(N[C@@H]2CCC(=O)NC2=O)cc1F]

In some embodiments, LBM is

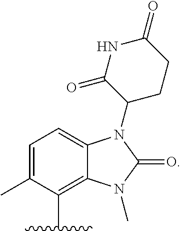 [SMILES: *c1c(C)ccc2c1n(C)c(=O)n2C1CCC(=O)NC1=O]

In some embodiments, LBM is

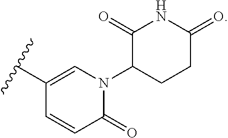 [SMILES: *c1ccc(=O)n(C2CCC(=O)NC2=O)c1]

In some embodiments, LBM is

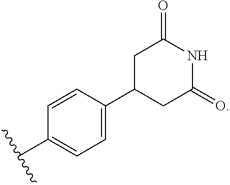 [SMILES: *c1ccc(C2CC(=O)NC(=O)C2)cc1]

In some embodiments, LBM is

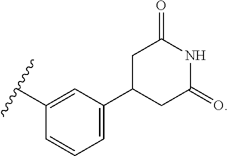 [SMILES: *c1cccc(C2CC(=O)NC(=O)C2)c1]

In some embodiments, LBM is

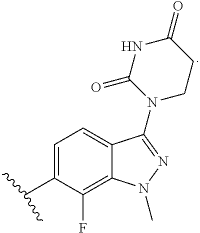 [SMILES: *c1ccc2c(N3CCC(=O)NC3=O)nn(C)c2c1F]

In some embodiments, LBM is

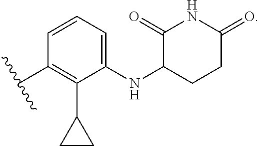 [SMILES: *c1cccc(NC2CCC(=O)NC2=O)c1C1CC1]

In some embodiments, LBM is

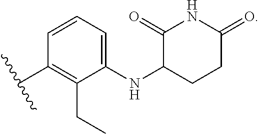 [SMILES: *c1cccc(NC2CCC(=O)NC2=O)c1CC]

In some embodiments, LBM is

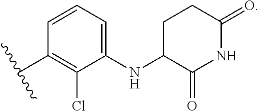 [SMILES: *c1cccc(NC2CCC(=O)NC2=O)c1Cl]

In some embodiments, LBM is

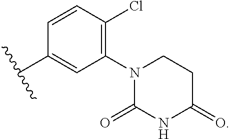 [SMILES: *c1ccc(Cl)c(N2CCC(=O)NC2=O)c1]

In some embodiments, LBM is
In some embodiments, LBM is

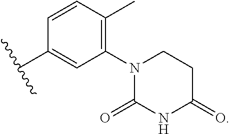 [SMILES: *c1ccc(C)c(N2CCC(=O)NC2=O)c1]

In some embodiments, LBM is

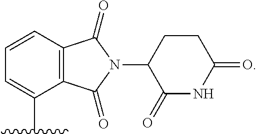 [SMILES: *c1cccc2c1C(=O)N(C1CCC(=O)NC1=O)C2=O]

In some embodiments, LBM is

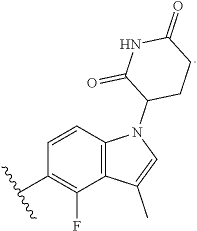 [SMILES: *c1ccc2c(c(C)cn2C2CCC(=O)NC2=O)c1F]

In some embodiments, LBM is

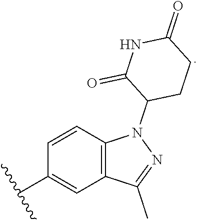 [SMILES: *c1ccc2c(c1)c(C)nn2C1CCC(=O)NC1=O]

In some embodiments, LBM is

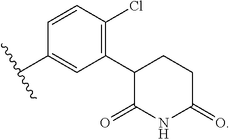 [SMILES: *c1ccc(Cl)c(C2CCC(=O)NC2=O)c1]

In some embodiments, LBM is

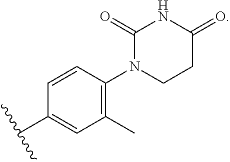 [SMILES: *c1ccc(N2CCC(=O)NC2=O)c(C)c1]

In some embodiments, LBM is

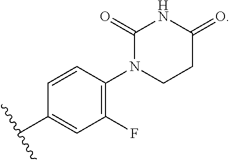 [SMILES: *c1ccc(N2CCC(=O)NC2=O)c(F)c1]

In some embodiments, LBM is

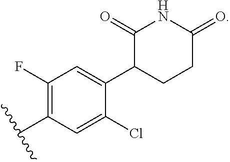 [SMILES: *c1cc(Cl)c(C2CCC(=O)NC2=O)cc1F]

In some embodiments, LBM is

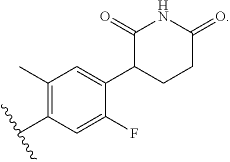 [SMILES: *c1cc(F)c(C2CCC(=O)NC2=O)cc1C]

In some embodiments, LBM is

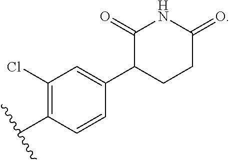 [SMILES: *c1ccc(C2CCC(=O)NC2=O)cc1Cl]

In some embodiments, LBM is

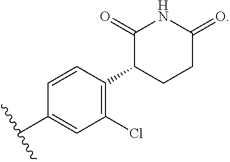 [SMILES: *c1ccc([C@H]2CCC(=O)NC2=O)c(Cl)c1]

In some embodiments,

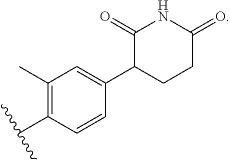 [SMILES: *c1ccc(C2CCC(=O)NC2=O)cc1C]

In some embodiments, LBM is

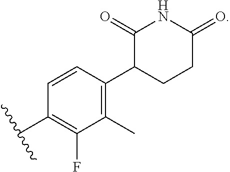 [SMILES: *c1ccc(C2CCC(=O)NC2=O)c(C)c1F]

In some embodiments, LBM is

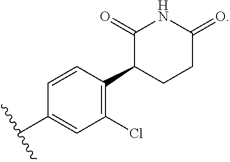 [SMILES: *c1ccc([C@@H]2CCC(=O)NC2=O)c(Cl)c1]

In some embodiments, LBM is

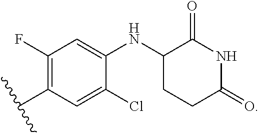 [SMILES: *c1cc(Cl)c(NC2CCC(=O)NC2=O)cc1F]

In some embodiments, LBM is

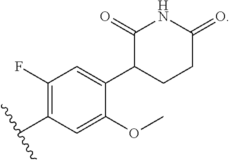 [SMILES: *c1cc(OC)c(C2CCC(=O)NC2=O)cc1F]

In some embodiments, LBM is

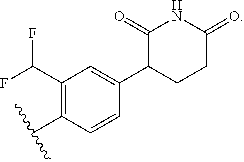 [SMILES: *c1ccc(C2CCC(=O)NC2=O)cc1C(F)F]

In some embodiments, LBM is

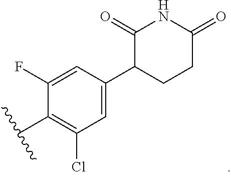 [SMILES: *c1c(F)cc(C2CCC(=O)NC2=O)cc1Cl]

In some embodiments, LBM is
In some embodiments, LBM is

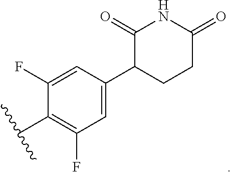 [SMILES: *c1c(F)cc(C2CCC(=O)NC2=O)cc1F]

In some embodiments, LBM is

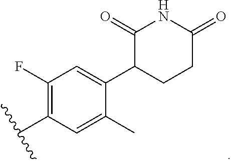 [SMILES: *c1cc(C)c(C2CCC(=O)NC2=O)cc1F]

In some embodiments, LBM is

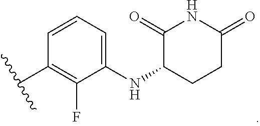 [SMILES: *c1cccc(N[C@H]2CCC(=O)NC2=O)c1F]

In some embodiments, LBM is

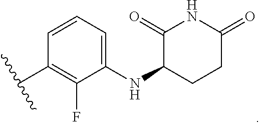 [SMILES: *c1cccc(N[C@@H]2CCC(=O)NC2=O)c1F]

In some embodiments, LBM is

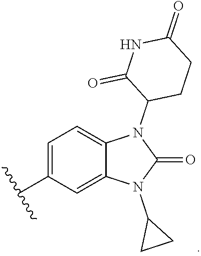 [SMILES: *c1ccc2c(c1)n(C1CC1)c(=O)n2C1CCC(=O)NC1=O]

In some embodiments, LBM is

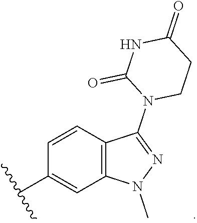 [SMILES: *c1ccc2c(N3CCC(=O)NC3=O)nn(C)c2c1]

In some embodiments, LBM is

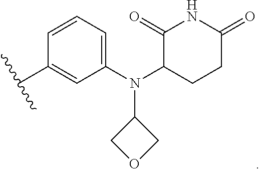 [SMILES: *c1cccc(N(C2COC2)C2CCC(=O)NC2=O)c1]

In some embodiments, LBM is

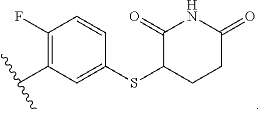 [SMILES: *c1cc(SC2CCC(=O)NC2=O)ccc1F]

In some embodiments, LBM is

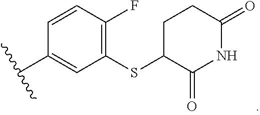 [SMILES: *c1ccc(F)c(SC2CCC(=O)NC2=O)c1]

In some embodiments, LBM is

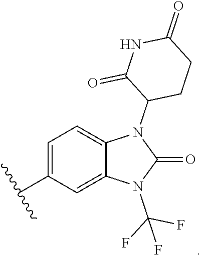 [SMILES: *c1ccc2c(c1)n(C(F)(F)F)c(=O)n2C1CCC(=O)NC1=O]

In some embodiments, LBM is

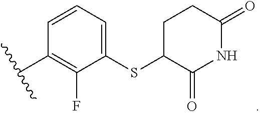 [SMILES: *c1cccc(SC2CCC(=O)NC2=O)c1F]

In some embodiments, LBM is

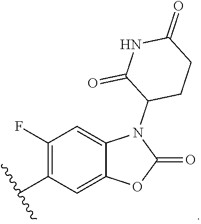 [SMILES: *c1cc2oc(=O)n(C3CCC(=O)NC3=O)c2cc1F]

In some embodiments, LBM is

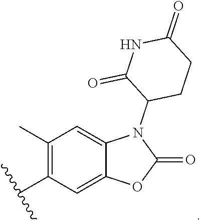 [SMILES: *c1cc2oc(=O)n(C3CCC(=O)NC3=O)c2cc1C]

In some embodiments, LBM is

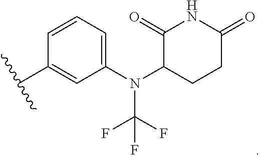 [SMILES: *c1cccc(N(C2CCC(=O)NC2=O)C(F)(F)F)c1]

In some embodiments, LBM is

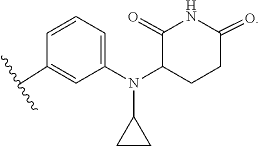 [SMILES: *c1cccc(N(C2CC2)C2CCC(=O)NC2=O)c1]

In some embodiments, LBM is

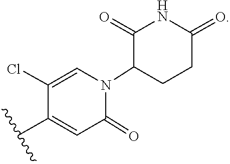 [SMILES: *c1cc(=O)n(C2CCC(=O)NC2=O)cc1Cl]

In some embodiments, LBM is

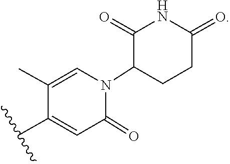 [SMILES: *c1cc(=O)n(C2CCC(=O)NC2=O)cc1C]

In some embodiments, LBM is

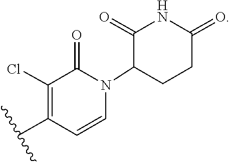 [SMILES: *c1ccn(C2CCC(=O)NC2=O)c(=O)c1Cl]

In some embodiments, LBM is

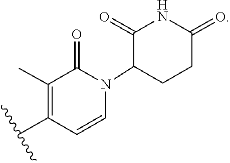 [SMILES: *c1ccn(C2CCC(=O)NC2=O)c(=O)c1C]

In some embodiments, LBM is

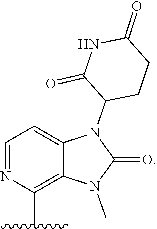 [SMILES: *c1nccc2c1n(C)c(=O)n2C1CCC(=O)NC1=O]

In some embodiments, LBM is

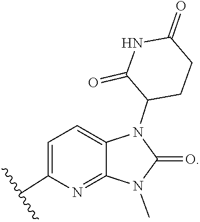 [SMILES: *c1ccc2c(n1)n(C)c(=O)n2C1CCC(=O)NC1=O]

In some embodiments, LBM is

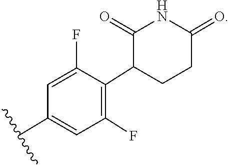 [SMILES: *c1cc(F)c(C2CCC(=O)NC2=O)c(F)c1]

In some embodiments, LBM is

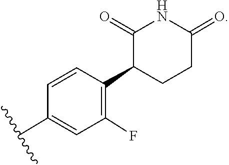 [SMILES: *c1ccc([C@@H]2CCC(=O)NC2=O)c(F)c1]

In some embodiments, LBM is

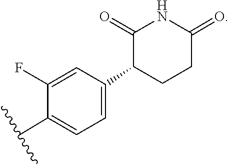 [SMILES: *c1ccc([C@H]2CCC(=O)NC2=O)cc1F]

In some embodiments, LBM is

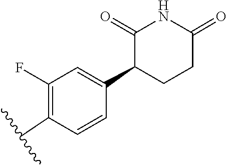 [SMILES: *c1ccc([C@@H]2CCC(=O)NC2=O)cc1F]

In some embodiments, LBM is

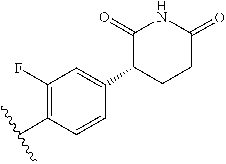 [SMILES: *c1ccc([C@H]2CCC(=O)NC2=O)cc1F]

In some embodiments, LBM is

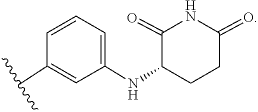 [SMILES: *c1cccc(N[C@H]2CCC(=O)NC2=O)c1]

In some embodiments, LBM is

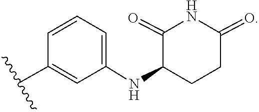 [SMILES: *c1cccc(N[C@@H]2CCC(=O)NC2=O)c1]

In some embodiments, LBM is

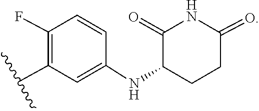 [SMILES: *c1cc(N[C@H]2CCC(=O)NC2=O)ccc1F]

In some embodiments, LBM is

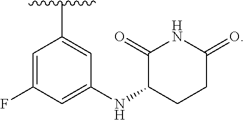 [SMILES: *c1cc(F)cc(N[C@H]2CCC(=O)NC2=O)c1]

In some embodiments, LBM is

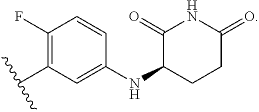 [SMILES: *c1cc(N[C@@H]2CCC(=O)NC2=O)ccc1F]

In embodiments, LBM is

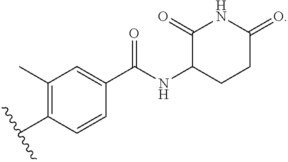 [SMILES: *c1ccc(C(=O)NC2CCC(=O)NC2=O)cc1C]

In some embodiments, LBM is

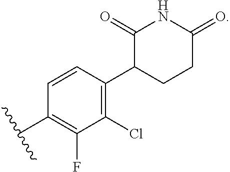 [SMILES: *c1ccc(C2CCC(=O)NC2=O)c(Cl)c1F]

In some embodiments, LBM is

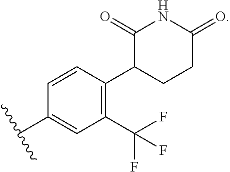 [SMILES: *c1ccc(C2CCC(=O)NC2=O)c(C(F)(F)F)c1]

In some embodiments, LBM is

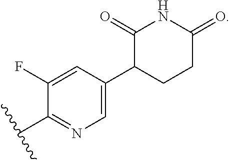 [SMILES: *c1ncc(C2CCC(=O)NC2=O)cc1F]

In some embodiments, LBM is

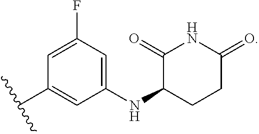 [SMILES: *c1cc(F)cc(N[C@@H]2CCC(=O)NC2=O)c1]

In some embodiments, LBM is

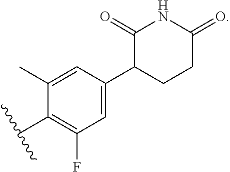 [SMILES: *c1c(C)cc(C2CCC(=O)NC2=O)cc1F]

In some embodiments, LBM is

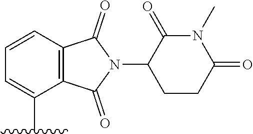 [SMILES: *c1cccc2c1C(=O)N(C1CCC(=O)N(C)C1=O)C2=O]

In some embodiments, LBM is

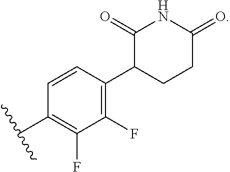 [SMILES: *c1ccc(C2CCC(=O)NC2=O)c(F)c1F]

In some embodiments, LBM is

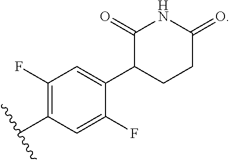 [SMILES: *c1cc(F)c(C2CCC(=O)NC2=O)cc1F]

In some embodiments, LBM is

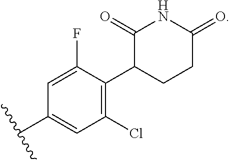 [SMILES: *c1cc(F)c(C2CCC(=O)NC2=O)c(Cl)c1]

In some embodiments, LBM is

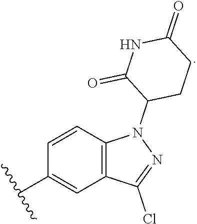 [SMILES: *c1ccc2c(c1)c(Cl)nn2C1CCC(=O)NC1=O]

In some embodiments, LBM is

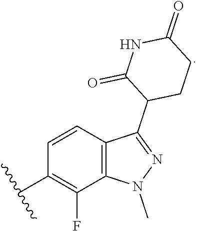 [SMILES: *c1ccc2c(C3CCC(=O)NC3=O)nn(C)c2c1F]

In some embodiments, LBM is

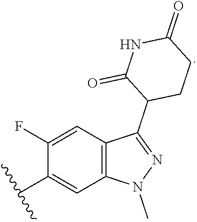 [SMILES: *c1cc2c(cc1F)c(C1CCC(=O)NC1=O)nn2C]

In some embodiments, LBM is

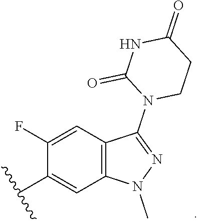 [SMILES: *c1cc2c(cc1F)c(N1CCC(=O)NC1=O)nn2C]

In some embodiments, LBM is

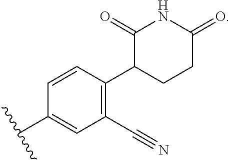 [SMILES: *c1ccc(C2CCC(=O)NC2=O)c(C#N)c1]

In some embodiments, LBM is

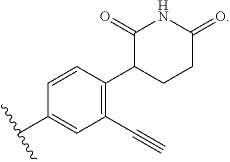 [SMILES: *c1ccc(C2CCC(=O)NC2=O)c(C#C)c1]

In some embodiments, LBM is

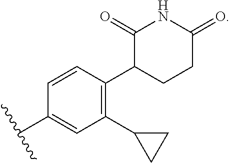 [SMILES: *c1ccc(C2CCC(=O)NC2=O)c(C2CC2)c1]

In some embodiments, LBM is

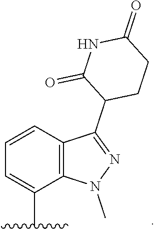 [SMILES: *c1cccc2c(C3CCC(=O)NC3=O)nn(C)c12]

In some embodiments, LBM is

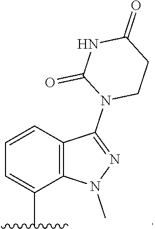 [SMILES: *c1cccc2c(N3CCC(=O)NC3=O)nn(C)c12]

In some embodiments, LBM is

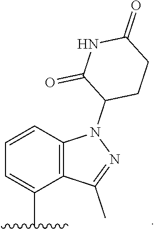 [SMILES: *c1cccc2c1c(C)nn2C1CCC(=O)NC1=O]

In some embodiments, LBM is selected from those in Table 1 below.

In some embodiments, the present invention provides the compound of formula I-a or I-a′, wherein DIM (e.g., LBM) is

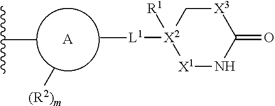

from formula I-aa, to provide a compound of formula I-a or I-a′-1:

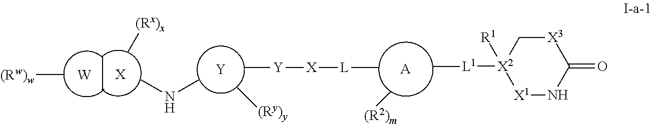

or a pharmaceutically acceptable salt thereof, wherein each of Y, X, X¹, X², X³, R¹, R², R^{w}, R^{x}, R^{y}, L, L¹, Ring A, Ring W, Ring X, Ring Y, m, w, x, and y is as defined above and described in embodiments herein, both singly and in combination.

In some embodiments, the present invention provides the compound of formula I-a-1, wherein Y is —S(0)₁₋₂—, X² is a carbon atom, X³ is —CH₂—, L¹ is a covalent bond, and Ring Y is phenylenyl as shown, to provide a compound of formula I-a-2:

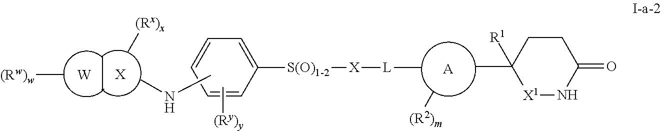

or a pharmaceutically acceptable salt thereof, wherein each of X¹, R¹, R², R^{w}, R^{x}, R^{y}, L, Ring A, Ring W, Ring X, m, w, x, and y is as defined above and described in embodiments herein, both singly and in combination.

In some embodiments, the present invention provides the compound of formula I-a-1, wherein Y is —S(O)₁₋₂—, X is

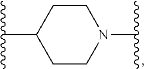 [SMILES: *C1CCN(*)CC1]

X² is a carbon atom, X³ are —CH₂—, L¹ is a covalent bond, and Ring Y is phenylenyl as shown, to provide a compound of formula I-a-3:

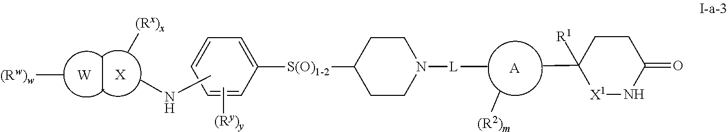

or a pharmaceutically acceptable salt thereof, wherein each of X¹, R¹, R², R^{w}, R^{x}, R^{y}, L, Ring A, Ring W, Ring X, m, w, x, and y is as defined above and described in embodiments herein, both singly and in combination.

In some embodiments, the present invention provides the compound of formula I-a-1, wherein Y is —S(O)₁₋₂—, X is

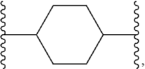 [SMILES: *C1CCC(*)CC1]

X² is a carbon atom, X³ are —CH₂—, L¹ is a covalent bond, and Ring Y is phenylenyl as shown, to provide a compound of formula I-a-4:

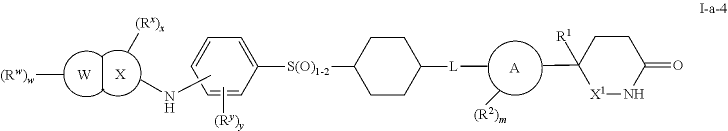

or a pharmaceutically acceptable salt thereof, wherein each of X¹, R¹, R², R^{w}, R^{x}, R^{y}, L, Ring A, Ring W, Ring X, m, w, x, and y is as defined above and described in embodiments herein, both singly and in combination.

In some embodiments the present invention provides the compound of formula I-a-1, wherein Y is —S(O)₁₋₂—, X is

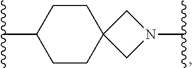 [SMILES: *C1CCC2(CC1)CN(*)C2]

X² is a carbon atom, X³ are —CH₂—, L¹ is a covalent bond, and Ring Y is phenylenyl as shown, to provide a compound of formula I-a-5:

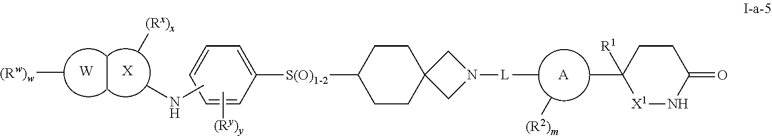

or a pharmaceutically acceptable salt thereof, wherein each of X¹, R¹, R², R^{w}, R^{x}, R^{y}, L, Ring A, Ring W, Ring X, m, w, x, and y is as defined above and described in embodiments herein, both singly and in combination.

In some embodiments, the present invention provides the compound of formula I-a-1, wherein Y is —S(O)₁₋₂—, X is

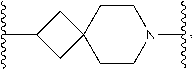 [SMILES: *C1CC2(CCN(*)CC2)C1]

X² is a carbon atom, X³ are —CH₂—, L¹ is a covalent bond, and Ring Y is phenylenyl as shown, to provide a compound of formula I-a-6:

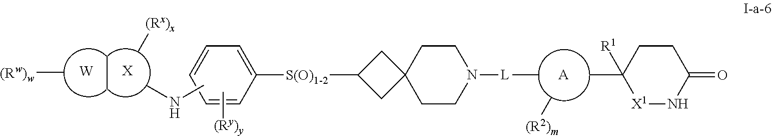

or a pharmaceutically acceptable salt thereof, wherein each of X¹, R¹, R², R^{w}, R^{x}, R^{y}, L, Ring A, Ring W, Ring X, m, w, x, and y is as defined above and described in embodiments herein, both singly and in combination.

In some embodiments, the present invention provides the compound of formula I-a-1, wherein Y is —S(O)(NH)—, X² is a carbon atom, X³ is —CH₂—, L¹ is a covalent bond, and Ring Y is phenylenyl as shown, to provide a compound of formula I-a-7:

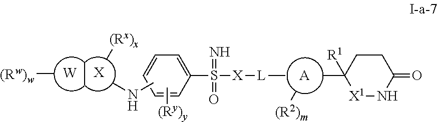

or a pharmaceutically acceptable salt thereof, wherein each of X¹, R¹, R², R^{w}, R^{x}, R^{y}, L, Ring A, Ring W, Ring X, m, w, x, and y is as defined above and described in embodiments herein, both singly and in combination.

In some embodiments, the present invention provides the compound of formula I-a-1, wherein Y is —S(O)₁₋₂—, X² is a carbon atom, X³ is —CH₂—, L¹ is a covalent bond, and Ring A is

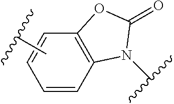

as shown, to provide a compound of formula I-a-8:

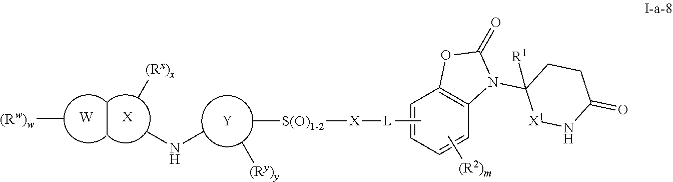

or a pharmaceutically acceptable salt thereof, wherein each of X¹, R¹, R², R^{w}, R^{x}, R^{y}, L, Ring W, Ring X, Ring Y, m, w, x, and y is as defined above and described in embodiments herein, both singly and in combination.

In some embodiments, the present invention provides the compound of formula I-a-1, wherein Y is —S(O)₁₋₂—, X² is a carbon atom, X³ is —CH₂—, L¹ is a covalent bond, Ring W is and

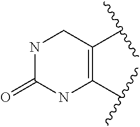 [SMILES: *C1=C(*)NC(=O)NC1]

as shown, to provide a compound of formula I-a-9:

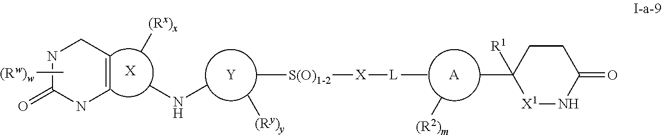

or a pharmaceutically acceptable salt thereof, wherein each of X¹, R¹, R², R^{w}, R^{x}, R^{y}, L, Ring A, Ring X, Ring Y, m, w, x, and y is as defined above and described in embodiments herein, both singly and in combination.

In some embodiments, the present invention provides the compound of formula I-a-1, wherein Y is —S(O)₁₋₂—, X² is a carbon atom, X³ is —CH₂—, L¹ is a covalent bond, and Ring W is

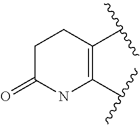 [SMILES: *C1=C(*)NC(=O)CC1]

as shown, to provide a compound of formula I-a-10:

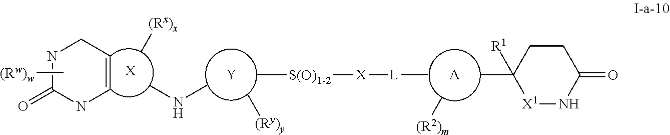

or a pharmaceutically acceptable salt thereof, wherein each of X¹, R¹, R², R^{w}, R^{x}, R^{y}, L, Ring A, Ring X, Ring Y, m, w, x, and y is as defined above and described in embodiments herein, both singly and in combination.

In some embodiments, the present invention provides the compound of formula I-a-1, wherein Y is —S(O)₁₋₂—, X² is a carbon atom, X³ is —CH₂—, L¹ is a covalent bond, and Ring X is pyridylenyl as shown, to provide a compound of formula I-a-11:

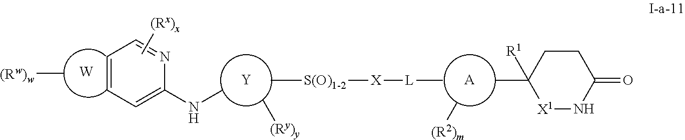

or a pharmaceutically acceptable salt thereof, wherein each of X¹, R¹, R², R^{w}, R^{x}, R^{y}, L, Ring A, Ring W, Ring Y, m, w, x, and y is as defined above and described in embodiments herein, both singly and in combination.

In some embodiments, the present invention provides the compound of formula I-a-1, wherein Y is —S(O)₁₋₂—, X² is a carbon atom, X³ is —CH₂—, L¹ is a covalent bond, and Ring X is pyridylenyl as shown, to provide a compound of formula I-a-12:

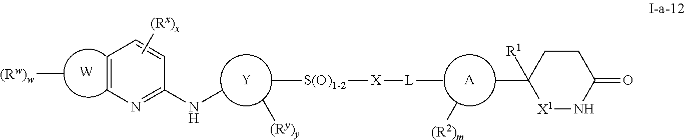

or a pharmaceutically acceptable salt thereof, wherein each of X¹, R¹, R², R^{w}, R^{x}, R^{y}, L, Ring A, Ring W, Ring Y, m, w, x, and y is as defined above and described in embodiments herein, both singly and in combination.

In some embodiments, the present invention provides the compound of formula I-a, wherein DIM (e.g., LBM) is

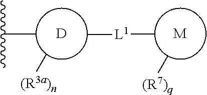

from formula I-nn, to provide a compound of formula I-a-20:

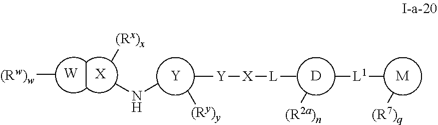

or a pharmaceutically acceptable salt thereof, wherein each of Y, X, R^{w}, R, R^{Y}, R^{3a}, R⁷, L, L¹, Ring D, Ring M, Ring W, Ring X, Ring Y, n, q, w, x, and y is as defined above and described in embodiments herein, both singly and in combination.

In some embodiments, the present invention provides the compound of formula I-a-20, wherein Ring M is

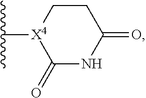

L¹ is a covalent bond, Y is —S(O)₁₋₂—, and Ring Y is phenylenyl as shown, to provide a compound of formula I-a-21:

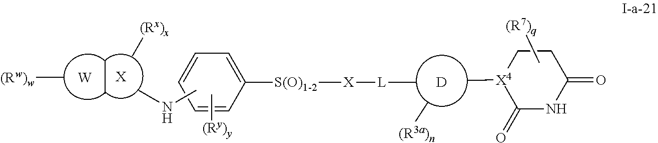

or a pharmaceutically acceptable salt thereof, wherein each of X, X⁴, R^{w}, R^{x}, R^{y}, R^{3a}, R⁷, L, Ring D, Ring W, Ring X, n, q, w, x, and y is as defined above and described in embodiments herein, both singly and in combination.

In some embodiments, the present invention provides the compound of formula I-a-20, wherein Ring M is

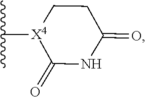

L¹ is a covalent bond, Y is —S(O)₁₋₂—, X is

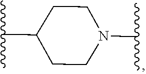 [SMILES: *C1CCN(*)CC1]

and Ring Y is phenylenyl as shown, to provide a compound of formula I-a-22:

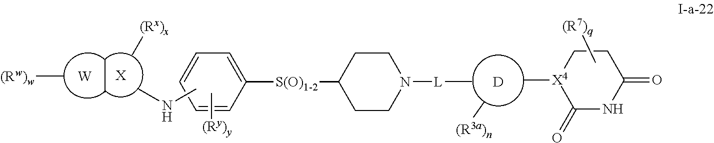

or a pharmaceutically acceptable salt thereof, wherein each of R^{w}, R^{x}, R^{y}, R^{3a}, R⁷, L, Ring D, Ring W, Ring X⁴, n, q, w, x, and y is as defined above and described in embodiments herein, both singly and in combination.

In some embodiments, the present invention provides the compound of formula I-a-20, wherein Ring M is

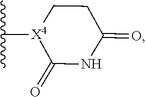

L¹ is a covalent bond, Y is —S(O)₁₋₂—, X is

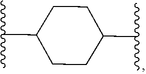 [SMILES: *C1CCC(*)CC1]

and Ring Y is phenylenyl as shown, to provide a compound of formula I-a-23:

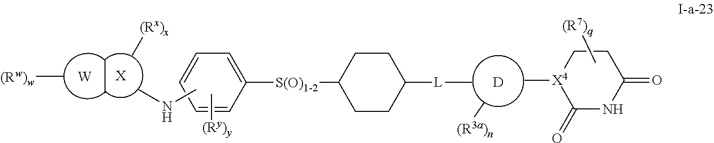

or a pharmaceutically acceptable salt thereof, wherein each of R^{w}, R^{x}, R^{y}, R^{3a}, R⁷, L, Ring D, Ring W, Ring X⁴, n, q, w, x, and y is as defined above and described in embodiments herein, both singly and in combination.

In some embodiments, the present invention provides the compound of formula I-a-20, wherein Ring M is

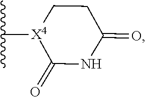

L¹ is a covalent bond, Y is —S(O)₁₋₂—, X is

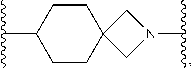 [SMILES: *C1CCC2(CC1)CN(*)C2]

and Ring Y is phenylenyl as shown, to provide a compound of formula I-a-24:

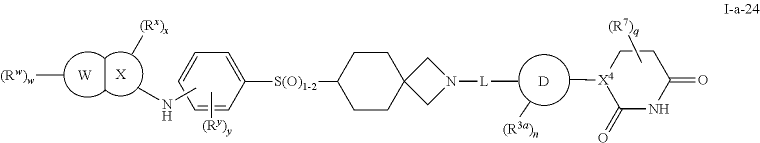

or a pharmaceutically acceptable salt thereof, wherein each of R^{w}, R^{x}, R^{y}, R^{3a}, R⁷, L, Ring D, Ring W, Ring X⁴, n, q, w, x, and y is as defined above and described in embodiments herein, both singly and in combination.

In some embodiments, the present invention provides the compound of formula I-a-20, wherein Ring M is

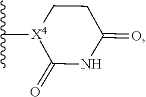

L¹ is a covalent bond, Y is —S(O)₁₋₂—, X is

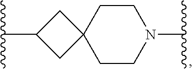 [SMILES: *C1CC2(CCN(*)CC2)C1]

and Ring Y is phenylenyl as shown, to provide a compound of formula I-a-25:

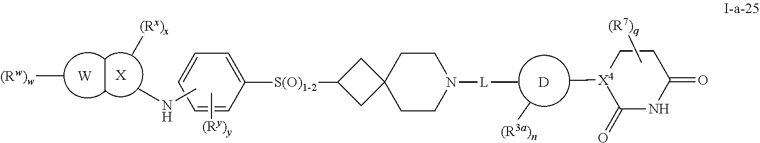

or a pharmaceutically acceptable salt thereof, wherein each of R^{w}, R^{x}, R^{y}, R^{3a}, R⁷, L, Ring D, Ring W, Ring X, n, q, w, x, and y is as defined above and described in embodiments herein, both singly and in combination.

In some embodiments, the present invention provides the compound of formula I-a-20, wherein Ring M is

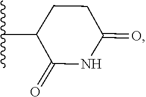 [SMILES: *C1CCC(=O)NC1=O]

L¹ is —NMe-, Y is —S(O)₁₋₂—, and Ring Y is phenylenyl as shown, to provide a compound of formula I-a-26:

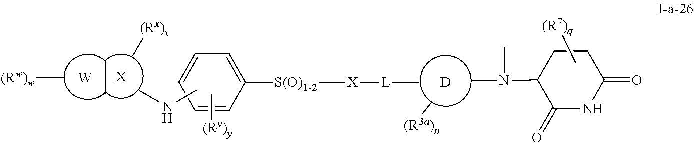

or a pharmaceutically acceptable salt thereof, wherein each of X, X⁴, R^{w}, R^{x}, R^{y}, R^{3a}, R⁷, L, Ring D, Ring W, Ring X, n, q, w, x, and y is as defined above and described in embodiments herein, both singly and in combination.

In some embodiments, the present invention provides the compound of formula I-a-20, wherein Ring M is

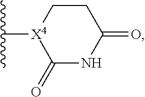

L¹ is a covalent bond, Y is —S(O)(NH)—, and Ring Y is phenylenyl as shown, to provide a compound of formula I-a-27:

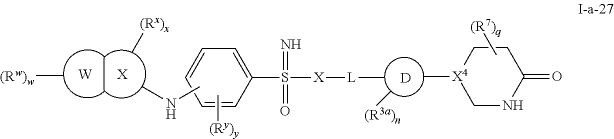

or a pharmaceutically acceptable salt thereof, wherein each of X, X⁴, R^{w}, R^{x}, R^{y}, R^{3a}, R⁷, L, Ring D, Ring W, Ring X, n, q, w, x, and y is as defined above and described in embodiments herein, both singly and in combination.

In some embodiments, the present invention provides the compound of formula I-a-20, wherein Ring M is

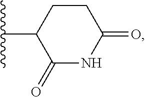 [SMILES: *C1CCC(=O)NC1=O]

L¹ is —NH—, Y is —S(O)(NH)—, and Ring Y is phenylenyl as shown, to provide a compound of formula I-a-28:

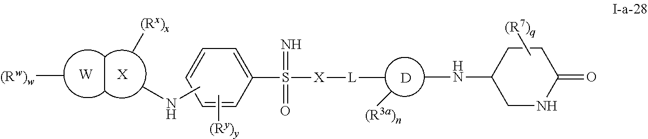

or a pharmaceutically acceptable salt thereof, wherein each of X, R^{w}, R^{x}, R^{y}, R^{3a}, R⁷, L, Ring D, Ring W, Ring X, n, q, w, x, and y is as defined above and described in embodiments herein, both singly and in combination.

In some embodiments, the present invention provides the compound of formula I-a-20, wherein Y is —S(O)₁₋₂—, Ring M is

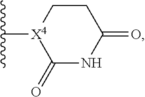

L¹ is a covalent bond, and Ring W is

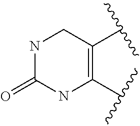 [SMILES: *C1=C(*)NC(=O)NC1]

as shown, to provide a compound of formula I-a-29:

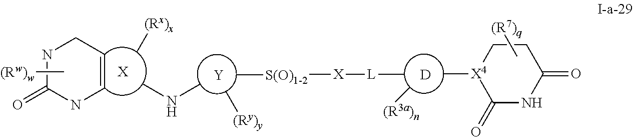

or a pharmaceutically acceptable salt thereof, wherein each of X, X⁴, R^{w}, R^{x}, R^{y}, R^{3a}, R⁷, L, Ring D, Ring X, Ring Y, n, q, w, x, and y is as defined above and described in embodiments herein, both singly and in combination.

In some embodiments, the present invention provides the compound of formula I-a-20, wherein Y is —S(O)₁₋₂—, Ring M is

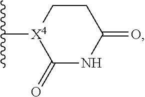

L¹ is a covalent bond, and Ring W is

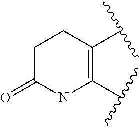 [SMILES: *C1=C(*)NC(=O)CC1]

as shown, to provide a compound of formula I-a-30:

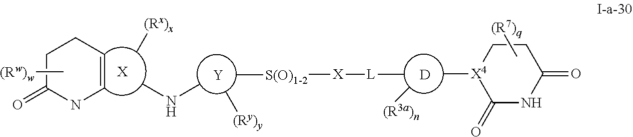

or a pharmaceutically acceptable salt thereof, wherein each of X, X⁴, R^{w}, R^{x}, R^{y}, R^{3a}, R⁷, L, Ring D, Ring X, Ring Y, n, q, w, x, and y is as defined above and described in embodiments herein, both singly and in combination.

In some embodiments, the present invention provides the compound of formula I-a-20, wherein Y is —S(O)₁₋₂—, Ring M is

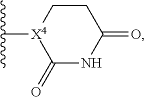

L¹ is a covalent bond, and Ring X is pyridylenyl as shown, to provide a compound of formula I-a-31:

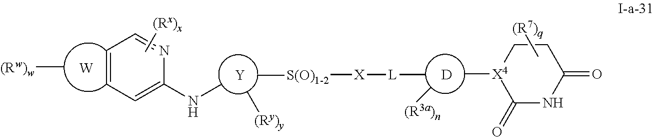

or a pharmaceutically acceptable salt thereof, wherein each of X, X⁴, R^{w}, R^{x}, R^{y}, R^{3a}, R⁷, L, Ring D, Ring W, Ring Y, n, q, w, x, and y is as defined above and described in embodiments herein, both singly and in combination.

In some embodiments, the present invention provides the compound of formula I-a-20, wherein Y is —S(O)₁₋₂—, Ring M is

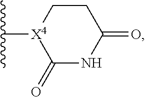

L¹ is a covalent bond, and Ring X is pyridylenyl as shown, to provide a compound of formula I-a-32:

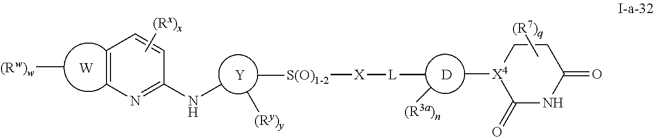

or a pharmaceutically acceptable salt thereof, wherein each of X, X⁴, R^{w}, R^{x}, R^{y}, R^{3a}, R⁷, L, Ring D, Ring W, Ring Y, n, q, w, x, and y is as defined above and described in embodiments herein, both singly and in combination.

In some embodiments, the present invention provides the compound of formula I-a-20, as a compound of formula I-a-33:

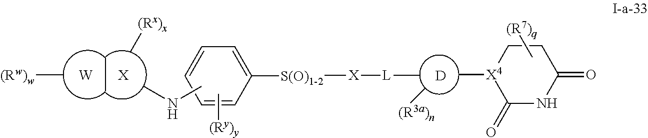

or a pharmaceutically acceptable salt thereof, wherein each of the variables is as defined above and described in embodiments herein, both singly and in combination.

In some embodiments, the present invention provides the compound of formula I-b or I-b′, wherein DIM (e.g., LBM) is

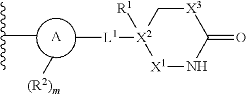

from formula I-aa, to provide a compound of formula I-b-1:

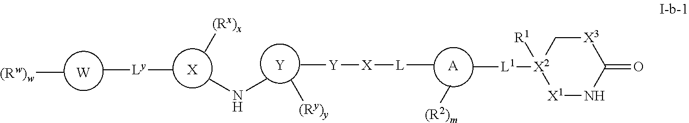

or a pharmaceutically acceptable salt thereof, wherein each of Y, X, X¹, X², X³, R¹, R², R^{w}, R^{x}, R^{y}, L, L¹, L, Ring A, Ring W, Ring X, Ring Y, m, v, w, x, and y is as defined above and described in embodiments herein, both singly and in combination.

In some embodiments, the present invention provides the compound of formula I-b-1, wherein Y is —S(O)₁₋₂—, X² is a carbon atom, X³ is —CH₂—, L is a covalent bond, and Ring Y is phenylenyl as shown, to provide a compound of formula I-b-2:

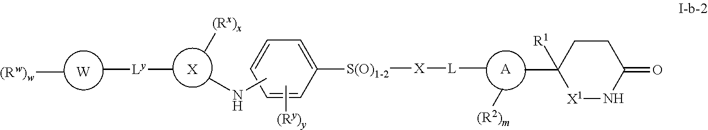

or a pharmaceutically acceptable salt thereof, wherein each of Y, X, X¹, R¹, R², R^{w}, R^{x}, R^{y}, L, L^{y}, Ring A, Ring X, Ring Y, m, w, x, and y is as defined above and described in embodiments herein, both singly and in combination.

In some embodiments, the present invention provides the compound of formula I-b-1, wherein Y is —S(O)₁₋₂—, X is

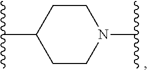 [SMILES: *C1CCN(*)CC1]

X² is a carbon atom, X³ is —CH₂—, L¹ is a covalent bond, and Ring Y is phenylenyl as shown, to provide a compound of formula I-b-3:

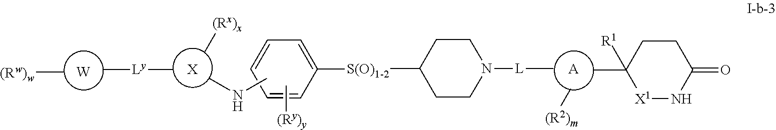

or a pharmaceutically acceptable salt thereof, wherein each of X¹, R¹, R², R^{w}, R^{x}, R^{y}, L, L, Ring A, Ring X, Ring Y, m, w, x, and y is as defined above and described in embodiments herein, both singly and in combination.

In some embodiments, the present invention provides the compound of formula I-b-1, wherein Y is —S(O)₁₂—, X is

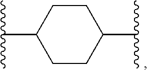 [SMILES: *C1CCC(*)CC1]

X² is a carbon atom, X³ is —CH₂—, L¹ is a covalent bond, and Ring Y is phenylenyl as shown, to provide a compound of formula I-b-4:

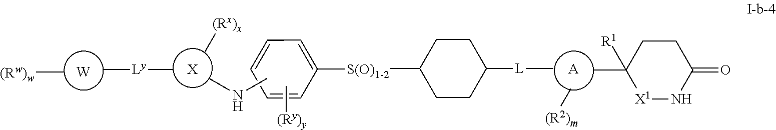

or a pharmaceutically acceptable salt thereof, wherein each of X¹, R¹, R², R^{w}, R^{x}, R^{y}, L, L^{y}, Ring A, Ring X, Ring Y, m, w, x, and y is as defined above and described in embodiments herein, both singly and in combination.

In some embodiments, the present invention provides the compound of formula I-b-1, wherein Y is —S(O)₁₋₂—, X is

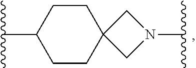 [SMILES: *C1CCC2(CC1)CN(*)C2]

X² is a carbon atom, X³ is —CH₂—, L¹ is a covalent bond, and Ring Y is phenylenyl as shown, to provide a compound of formula I-b-5:

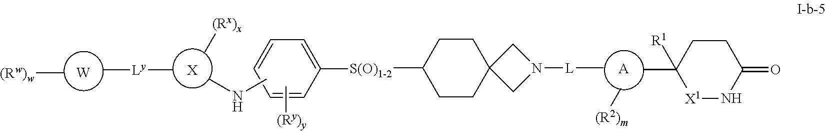

or a pharmaceutically acceptable salt thereof, wherein each of X¹, R¹, R², R^{w}, R^{x}, R^{y}, L, L, Ring A, Ring X, Ring Y, m, w, x, and y is as defined above and described in embodiments herein, both singly and in combination.

In some embodiments, the present invention provides the compound of formula I-b-1, wherein Y is —S(O)₁₋₂—, X is

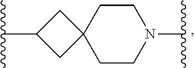 [SMILES: *C1CC2(CCN(*)CC2)C1]

X² is a carbon atom, X³ is —CH₂—, L¹ is a covalent bond, and Ring Y is phenylenyl as shown, to provide a compound of formula I-b-6:

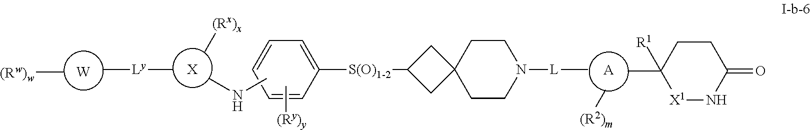

or a pharmaceutically acceptable salt thereof, wherein each of X¹, R¹, R², R^{w}, R^{x}, R^{y}, L, L, Ring A, Ring X, Ring Y, m, w, x, and y is as defined above and described in embodiments herein, both singly and in combination.

In some embodiments, the present invention provides the compound of formula I-b-1, wherein Y is —S(O)(NH)—, X² is a carbon atom, X³ is —CH₂—, L¹ is a covalent bond, and Ring Y is phenylenyl as shown, to provide a compound of formula I-b-7:

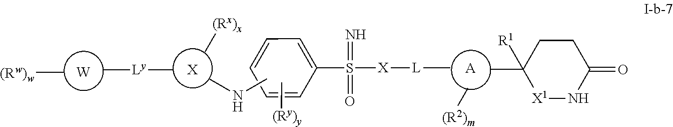

or a pharmaceutically acceptable salt thereof, wherein each of X, X¹, R¹, R², R^{w}, R^{x}, R^{y}, L, L, Ring A, Ring X, Ring Y, m, w, x, and y is as defined above and described in embodiments herein, both singly and in combination.

In some embodiments, the present invention provides the compound of formula I-b-1, wherein Y is —S(O)₁₋₂—, X² is a carbon atom, X³ is —CH₂—, L¹ is a covalent bond, and Ring A is

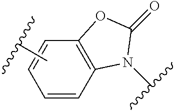

as shown, to provide a compound of formula I-b-8:

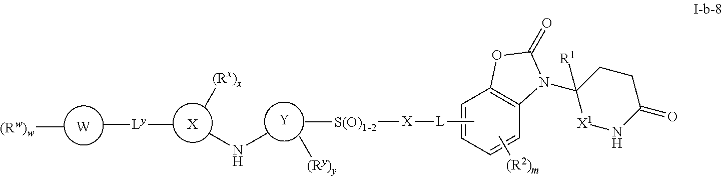

or a pharmaceutically acceptable salt thereof, wherein each of X, X¹, R¹, R², R^{w}, R^{x}, R^{y}, L, L, Ring W, Ring X, Ring Y, m, w, x, and y is as defined above and described in embodiments herein, both singly and in combination.

In some embodiments, the present invention provides the compound of formula I-b or I-b′, wherein DIM (e.g., LBM) is

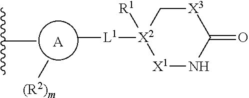

and Y is —S(O)₂₂NH—, from formula I-aa, to provide a compound of formula I-b-9:

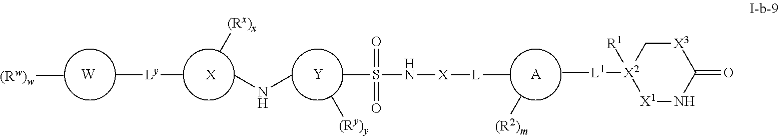

or a pharmaceutically acceptable salt thereof, wherein each of X¹, X², X³, R¹, R², R^{w}, R^{x}, R^{y}, L, L¹, L, Ring A, Ring W, Ring X, Ring Y, m, v, w, x, and y is as defined above and described in embodiments herein, both singly and in combination, and wherein X is an optionally substituted ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur.

In some embodiments, the present invention provides the compound of formula I-b or I-b′, wherein DIM (e.g., LBM) is

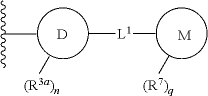

from formula I-nn, to provide a compound of formula I-b-10:

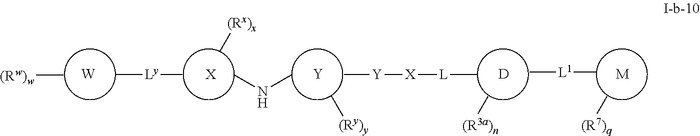

or a pharmaceutically acceptable salt thereof, wherein each of Y, X, R^{w}, R^{x}, R^{y}, R^{3a}, R⁷, L, L¹, L, Ring D, Ring M, Ring W, Ring X, Ring Y, n, q, w, x, and y is as defined above and described in embodiments herein, both singly and in combination.

In some embodiments, the present invention provides the compound of formula I-b-10, wherein Ring M is

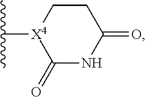

L¹ is a covalent bond, Y is —S(O)₁₋₂—, and Ring Y is phenylenyl as shown, to provide a compound of formula I-b-11:

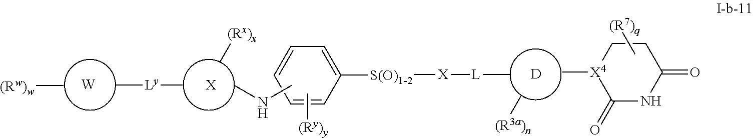

or a pharmaceutically acceptable salt thereof, wherein each of X, X⁴, R^{w}, R^{x}, R^{Y}, R^{3a}, R⁷, L, L, Ring D, Ring W, Ring X, n, q, w, x, and y is as defined above and described in embodiments herein, both singly and in combination.

In some embodiments, the present invention provides the compound of formula I-b-10, wherein Ring M is

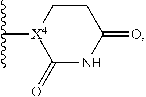

L¹ is a covalent bond, Y is —S(O)₁₋₂—, X is

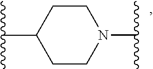 [SMILES: *C1CCN(*)CC1]

and Ring Y is phenylenyl as shown, to provide a compound of formula I-b-12:

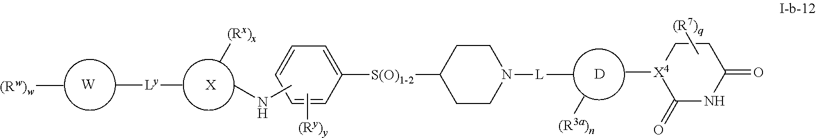

or a pharmaceutically acceptable salt thereof, wherein each of X, R^{w}, R^{x}, R^{Y}, R^{3a}, R⁷, L, L^{y}, Ring D, Ring W, Ring X, n, q, w, x, and y is as defined above and described in embodiments herein, both singly and in combination.

In some embodiments, the present invention provides the compound of formula I-b-10, wherein Ring M is

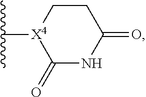

L¹ is a covalent bond, Y is —S(O)₁₂—, X is

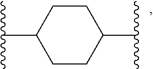 [SMILES: *C1CCC(*)CC1]

and Ring Y is phenylenyl as shown, to provide a compound of formula I-b-13:

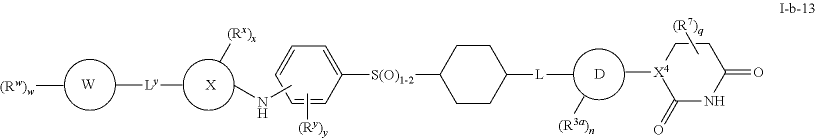

or a pharmaceutically acceptable salt thereof, wherein each of R^{w}, R^{x}, R^{y}, R^{3a}, R⁷, L, L^{y}, Ring D, Ring W, Ring X, n, q, w, x, and y is as defined above and described in embodiments herein, both singly and in combination.

In some embodiments, the present invention provides the compound of formula I-b-10, wherein Ring M is

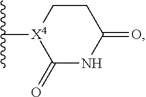

L¹ is a covalent bond, Y is —S(O)₁₋₂—, X is

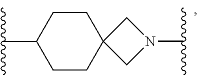 [SMILES: *C1CCC2(CC1)CN(*)C2]

and Ring Y is phenylenyl as shown, to provide a compound of formula I-b-14:

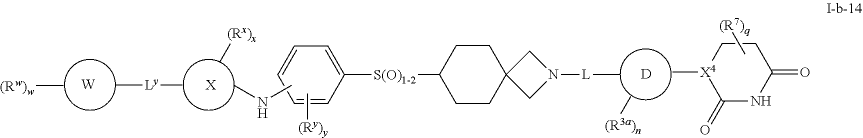

or a pharmaceutically acceptable salt thereof, wherein each of R^{w}, R^{x}, R^{y}, R^{3a}, R⁷, L, L, Ring D, Ring W, Ring X, n, q, w, x, and y is as defined above and described in embodiments herein, both singly and in combination.

In some embodiments, the present invention provides the compound of formula I-b-10, wherein Ring M is

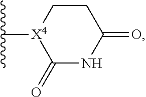

L¹ is a covalent bond, Y is —S(O)₁₋₂—, X is

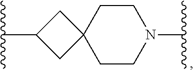 [SMILES: *C1CC2(CCN(*)CC2)C1]

and Ring Y is phenylenyl as shown, to provide a compound of formula I-b-15:

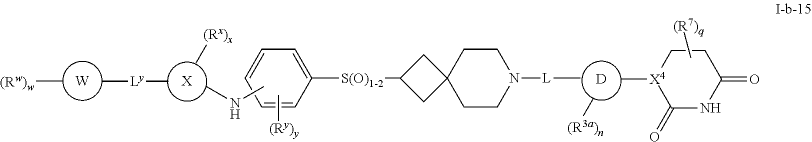

or a pharmaceutically acceptable salt thereof, wherein each of R^{w}, R^{x}, R^{y}, R^{3a}, R⁷, L, L, Ring D, Ring W, Ring X, n, q, w, x, and y is as defined above and described in embodiments herein, both singly and in combination.

In some embodiments, the present invention provides the compound of formula I-b-10, wherein Ring M is

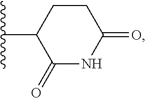 [SMILES: *C1CCC(=O)NC1=O]

L¹ is —NMe-, Y is —S(O)₁₋₂—, and Ring Y is phenylenyl as shown, to provide a compound of formula I-b-16:

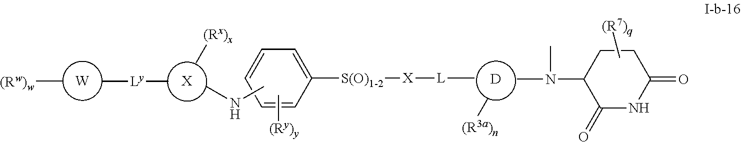

or a pharmaceutically acceptable salt thereof, wherein each of R^{w}, R^{x}, R^{y}, R^{3a}, R⁷, L, L, Ring D, Ring W, Ring X, n, q, w, x, and y is as defined above and described in embodiments herein, both singly and in combination.

In some embodiments, the present invention provides the compound of formula I-b-10, wherein Ring M is

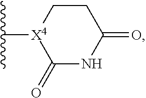

L¹ is a covalent bond, Y is —S(O)(NH)—, and Ring Y is phenylenyl as shown, to provide a compound of formula I-b-17:

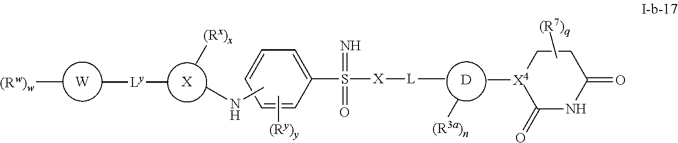

or a pharmaceutically acceptable salt thereof, wherein each of X, R^{w}, R^{x}, R^{Y}, R^{3a}, R⁷, L, L, Ring D, Ring W, Ring X, n, q, w, x, and y is as defined above and described in embodiments herein, both singly and in combination.

In some embodiments, the present invention provides the compound of formula I-b-10, wherein Ring M is

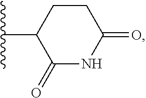 [SMILES: *C1CCC(=O)NC1=O]

L¹ is —NH—, Y is —S(O)(NH)—, and Ring Y is phenylenyl as shown, to provide a compound of formula I-b-18:

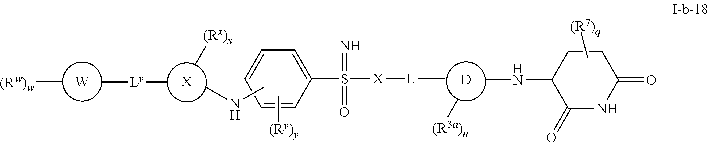

or a pharmaceutically acceptable salt thereof, wherein each of X, R^{w}, R^{x}, R^{y}, R^{3a}, R⁷, L, L^{y}, Ring D, Ring W, Ring X, n, q, w, x, and y is as defined above and described in embodiments herein, both singly and in combination.

In some embodiments, the present invention provides a compound formula I-b-10 of any one of the following formulae:

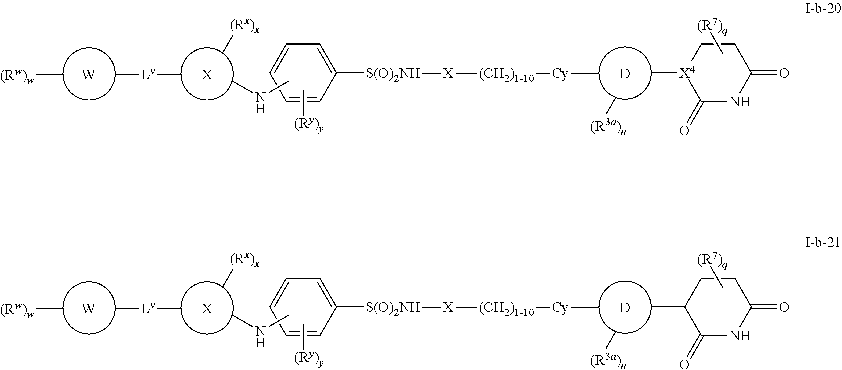

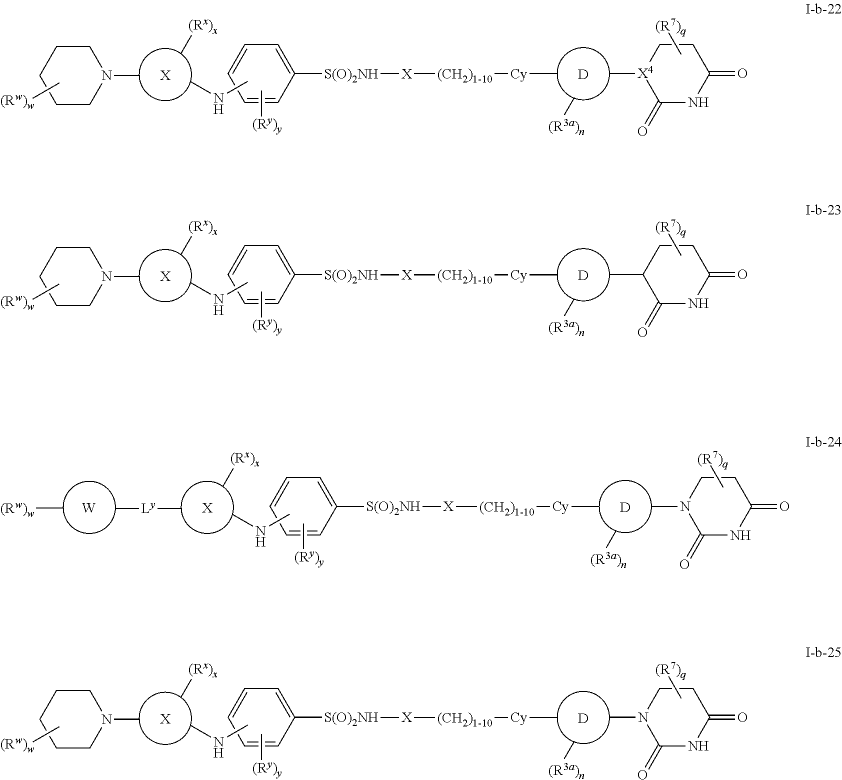

or a pharmaceutically acceptable salt thereof, wherein each of the variables is as defined above and described in embodiments herein, both singly and in combination.

In some embodiments, the present invention provides a compound of formula I-b-10 of any one of the following formulae:

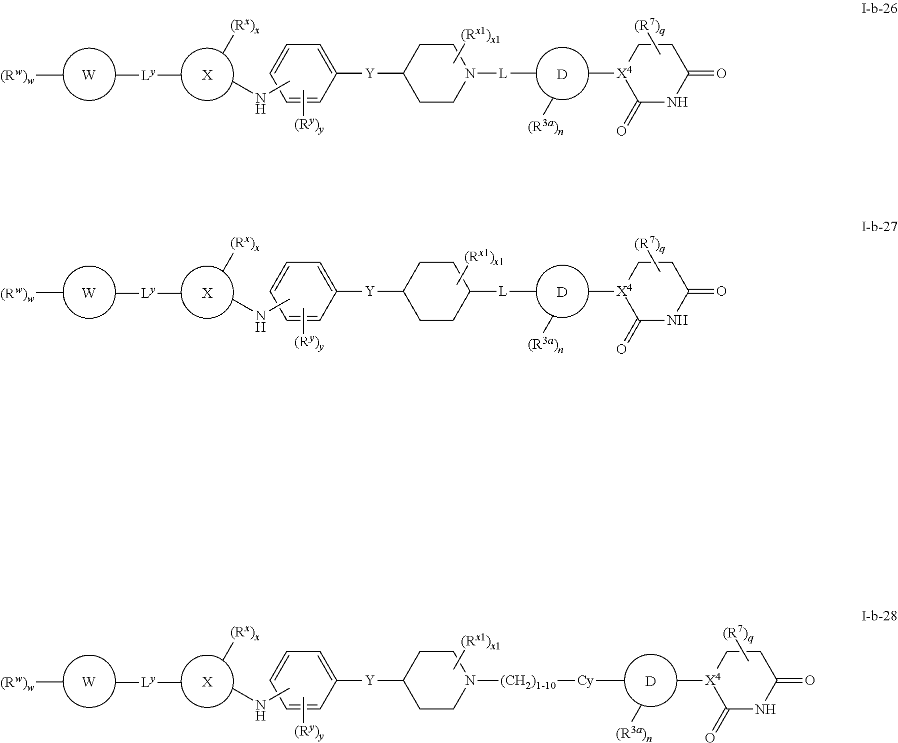

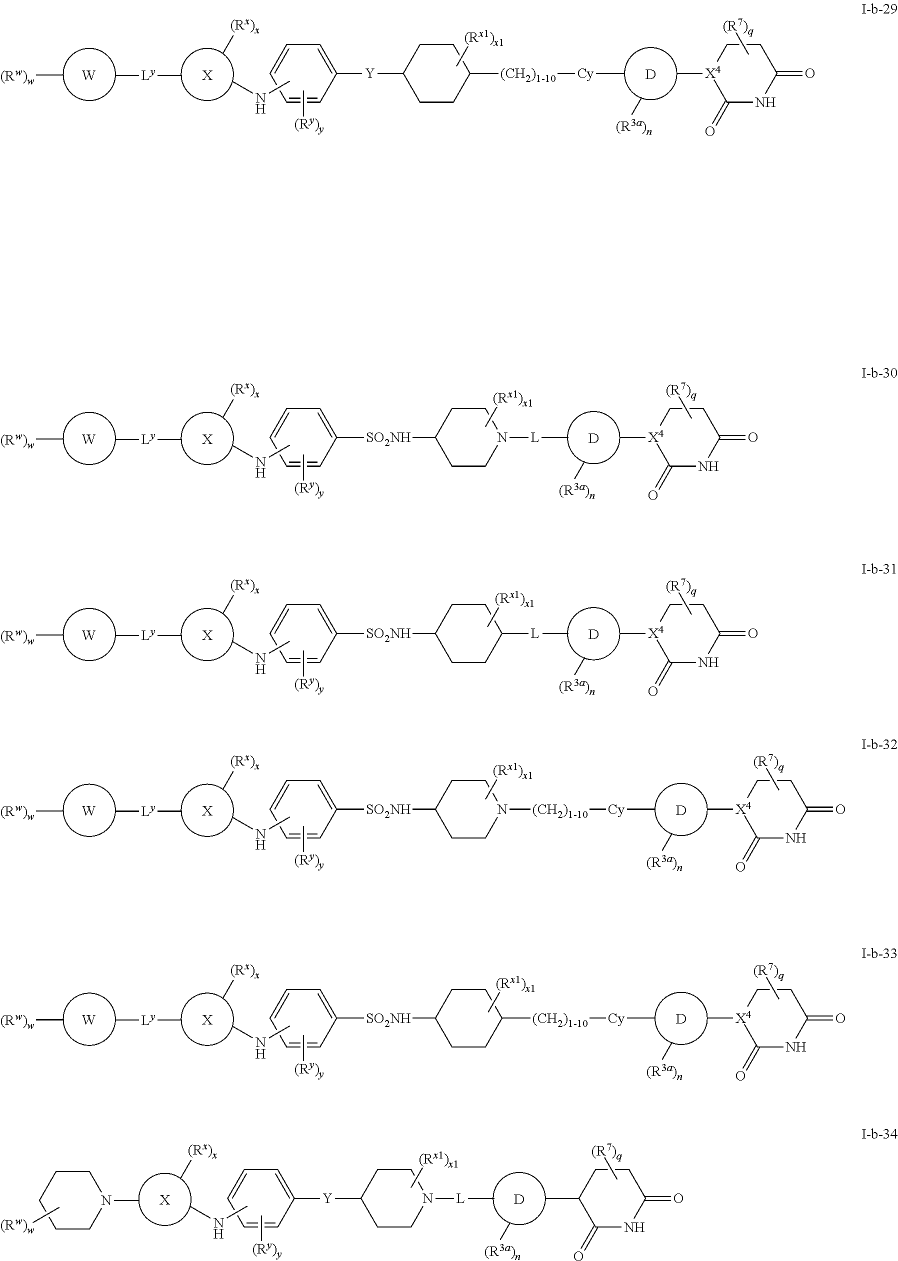

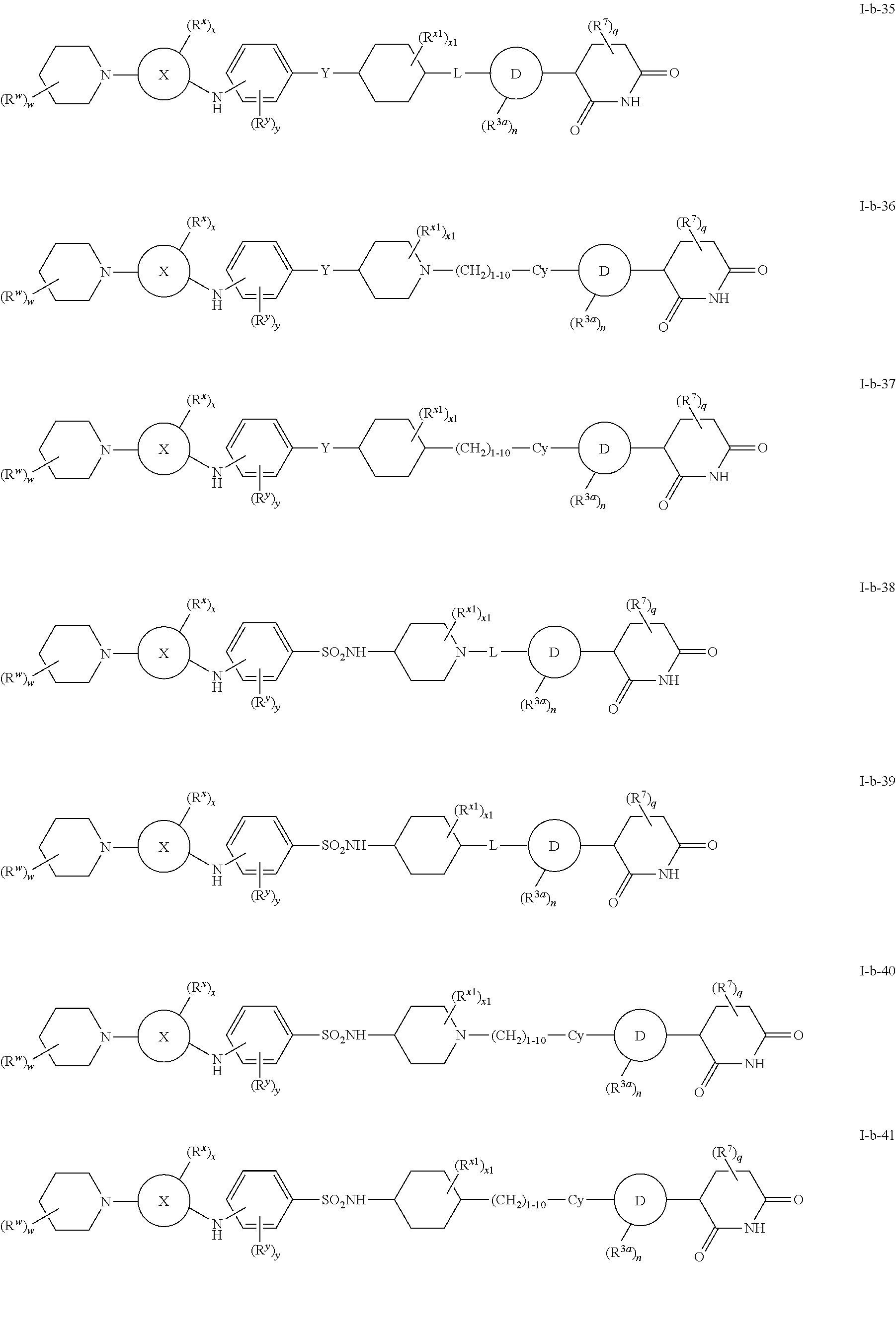

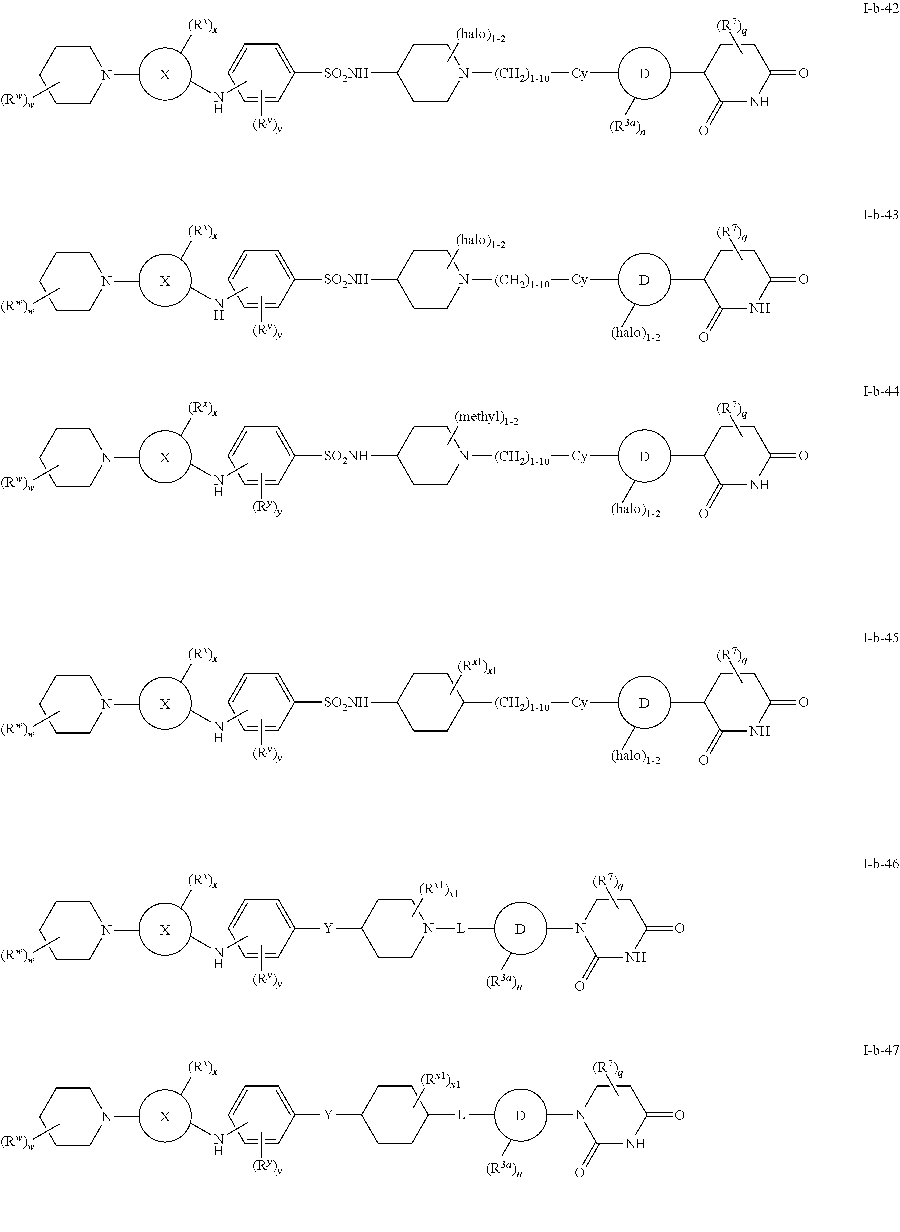

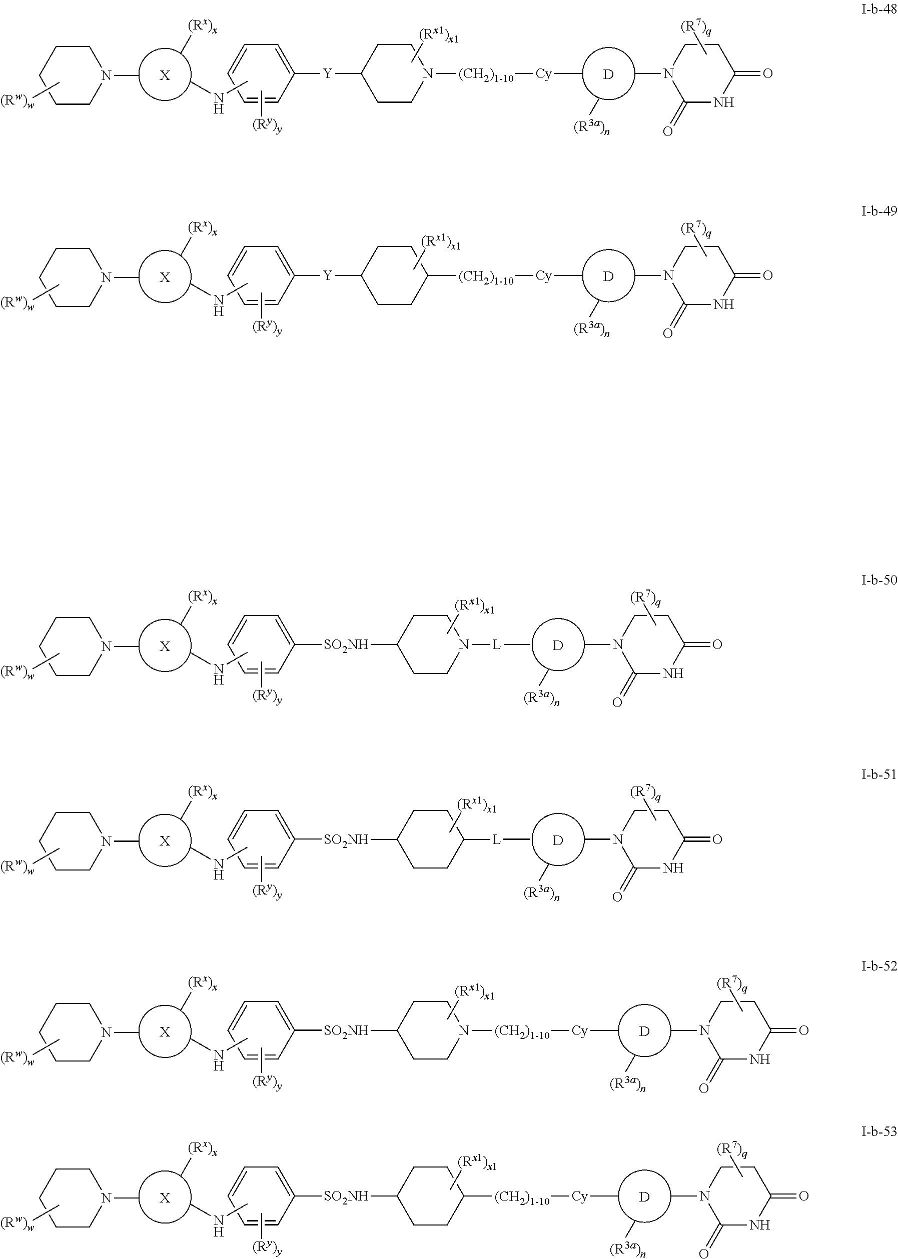

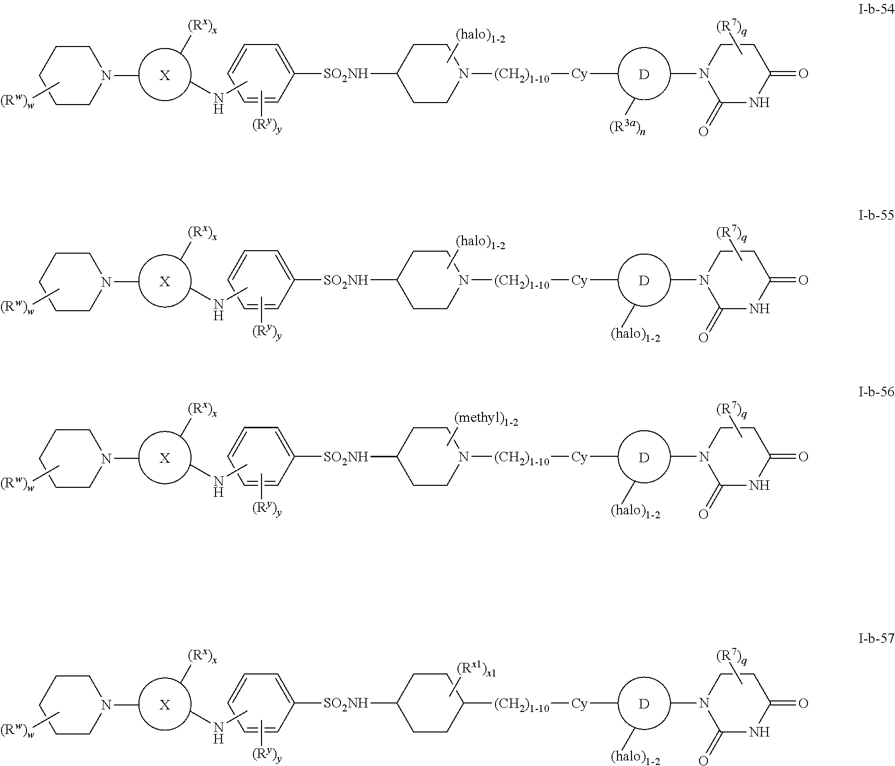

or a pharmaceutically acceptable salt thereof, wherein each of the variables is as defined above and described in embodiments herein, both singly and in combination, and wherein:
R^{x1} is hydrogen, halogen, —CN, C₁₋₆ alkyl, C₁₋₆ haloalkyl, —OH, —OC₁₋₆ alkyl, —OC₁₋₆ haloalkyl; and x1 is 0, 1, or 2.

As defined generally above, R^{x}i is hydrogen, halogen, —CN, C₁₋₆ alkyl, C₁₋₆ haloalkyl, —OH, —OC₁₋₆ alkyl, —OC₁₋₆ haloalkyl.

In some embodiments, R^{x1} is hydrogen. In some embodiments, R^{x1} is halogen. In some embodiments, R^{x1} is —CN. In some embodiments, R^{x1} is C₁₋₆ alkyl. In some embodiments, R^{x1} is C₁₋₆ haloalkyl. In some embodiments, R^{x1} is —OH. In some embodiments, R^{x1} is —OC₁₋₆ alkyl. In some embodiments, R^{x1} is —OC₁₋₆ haloalkyl.

In some embodiments, R^{x1} is fluoro or methyl.

In some embodiments, R^{x1} is selected from those depicted in Table 1, below.

As defined generally above, x1 is 0, 1, or 2.

In some embodiments, x1 is 0. In some embodiments, x1 is 1. In some embodiments, x1 is 2. In some embodiments, x1 is 0 or 1. In some embodiments, x1 is 1 or 2.

In some embodiments, x1 is selected from those depicted in Table 1, below.

In some embodiments, the present invention provides the compound of formula I-b-10 as a compound of formula I-b-58:

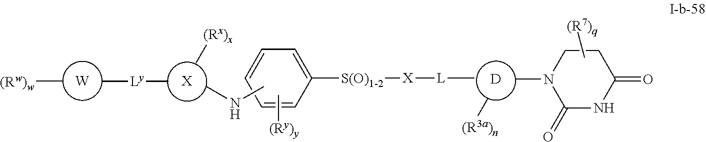

or a pharmaceutically acceptable salt thereof, wherein each of the variables is as defined above and described in embodiments herein, both singly and in combination.

In some embodiments, the present invention provides the compound of formula I-d, wherein DIM (e.g., LBM) is

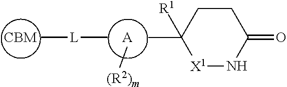

from formula I-cc, to provide a compound of formula I-d-1:

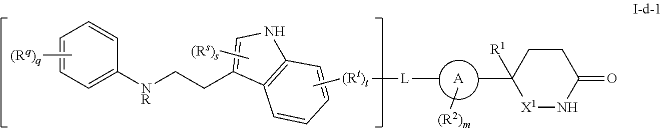

or a pharmaceutically acceptable salt thereof, wherein each of R^{q}, R^{s}, R^{t}, R, q, s, t, L, ring A, R², R¹, X¹, and m is as defined above and described in embodiments herein, both singly and in combination.

In certain embodiments, the present invention provides a compound of Formula I, wherein DIM (e.g., LBM) is an E3 ubiquitin ligase (cereblon) binding moiety thereby forming a compound of formula I-oo-1, I-oo-2, I-oo-3, I-oo-4, I-oo-5, I-oo-6, I-oo-7, I-oo-8, I-oo-9, or I-oo-10 respectively:

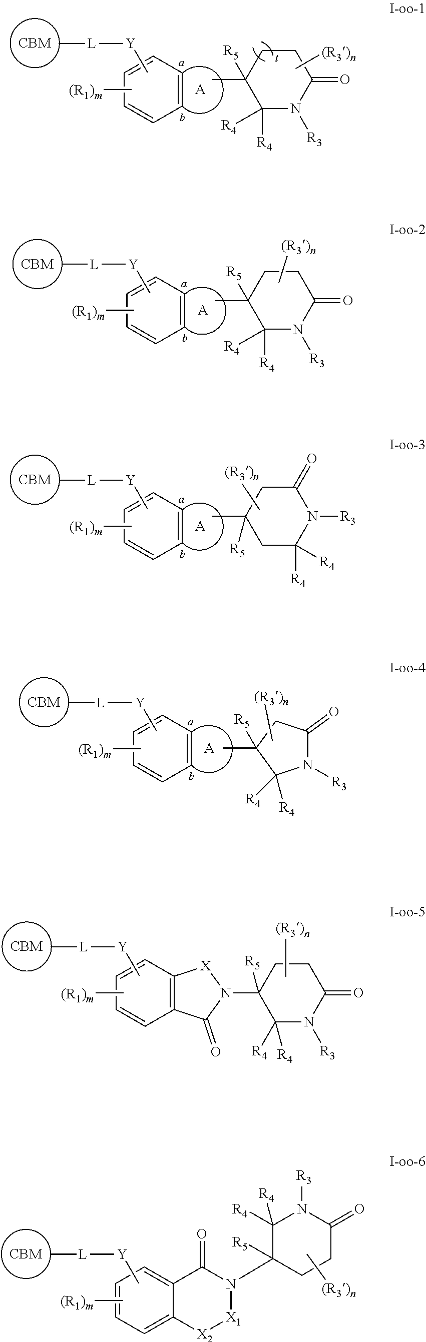

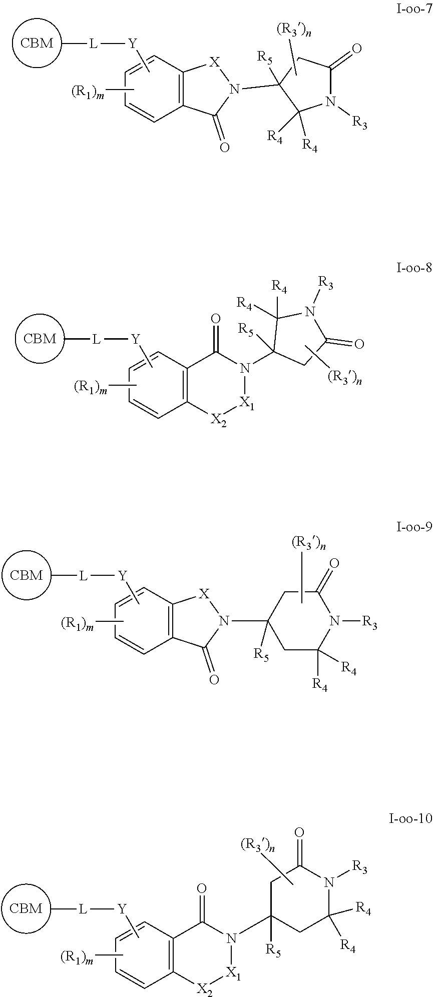

or a compound of formula I-oo′-1, I-oo′-2, I-oo′-3, I-oo′-4, I-oo′-5, I-oo′-6, I-oo′-7, I-oo′-8, I-oo′-9, or I-oo′-10 respectively:

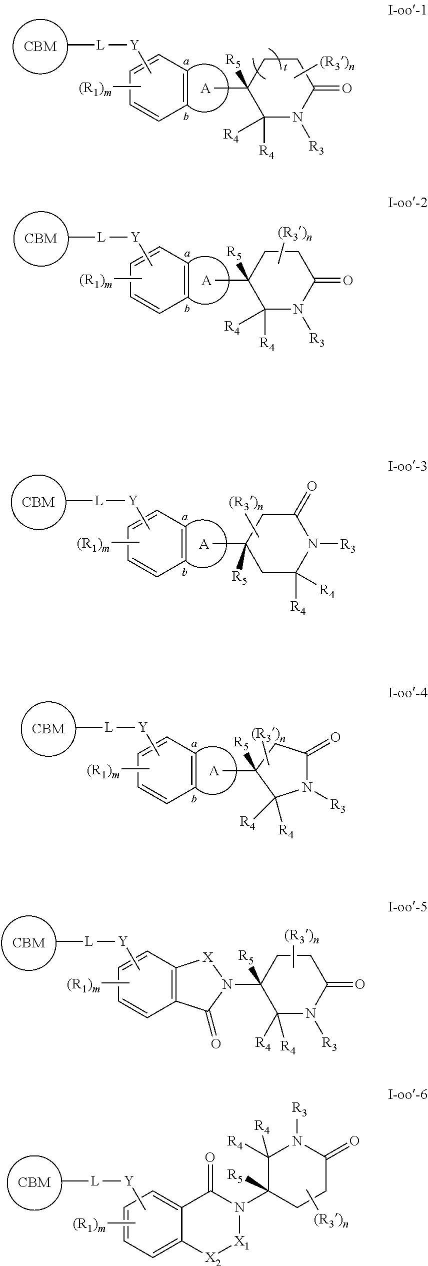

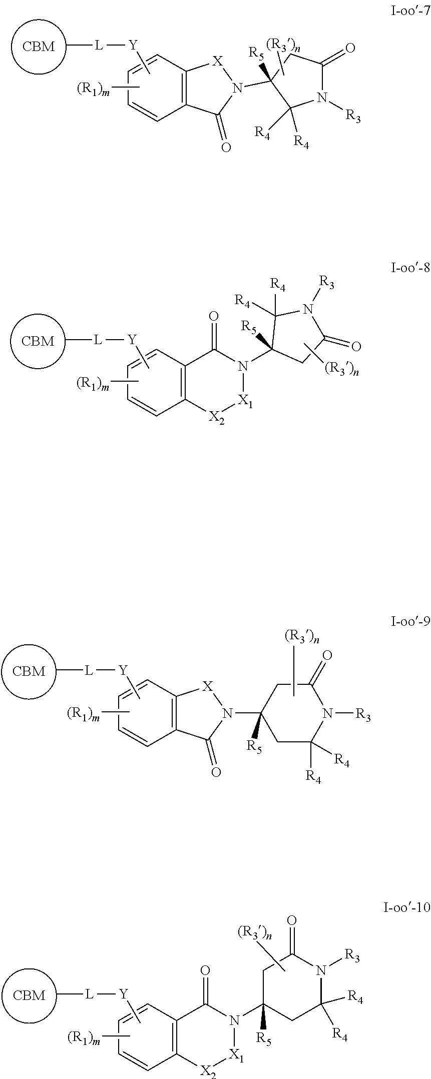

or a compound of formula I-oo″-1, I-oo″-2, I-oo″-3, I-oo″-4, I-oo″-5, I-oo″-6, I-oo″-7, I-oo″-8, I-oo″-9, or I-oo″-10 respectively:

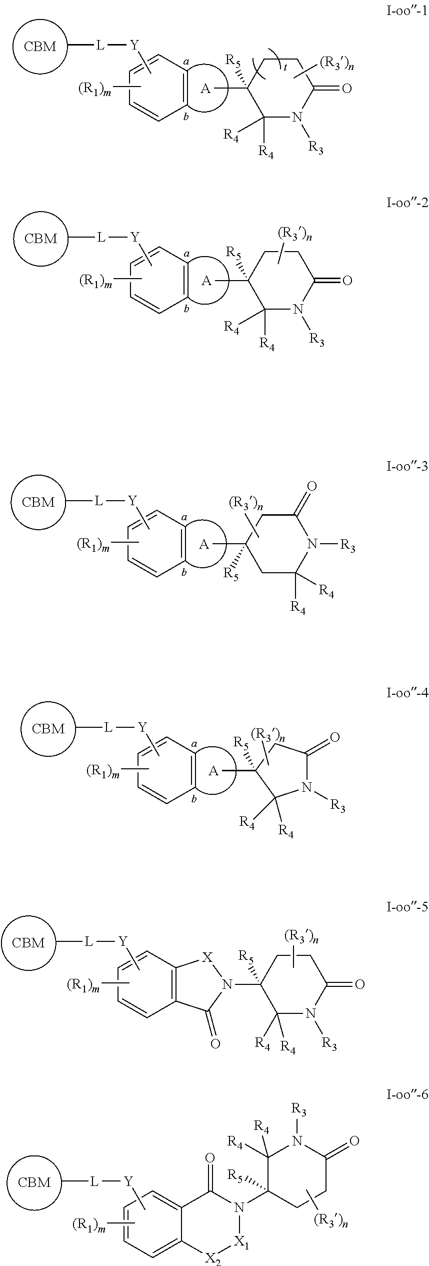

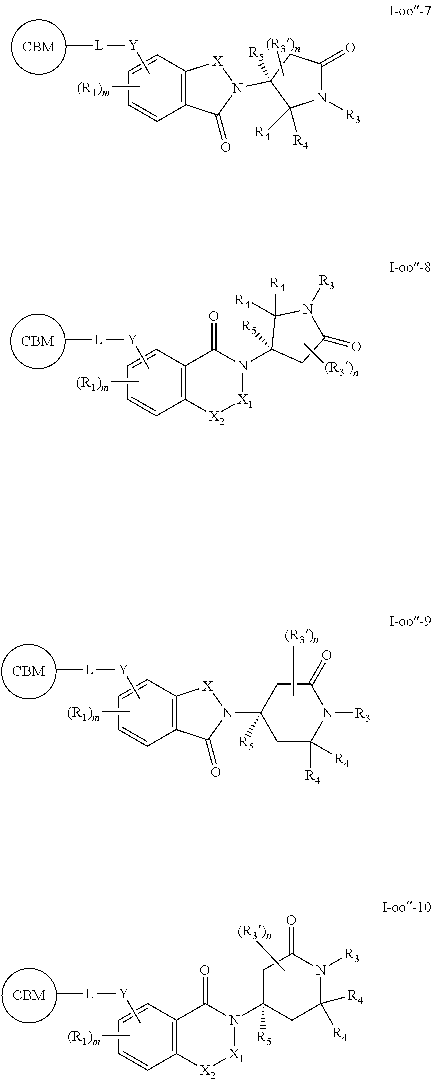

or a pharmaceutically acceptable salt thereof, wherein L and CBM are as defined above and described in embodiments herein, and wherein each of the variables

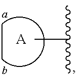

X, X₁, X₂, Y, R₁, R₃, R₃′, R₄, R₅, t, m and n is as defined and described in WO 2017/007612 and US 2018/0134684, the entirety of each of which is herein incorporated by reference.

Accordingly in some embodiments, the present invention provides a compound of formula I-oo-1, I-oo-2, I-oo-3, I-oo-4, I-oo-5, I-oo-6, I-oo-7, I-oo-8, I-oo-9, I-oo-10, I-oo′-1, I-oo′-2, I-oo′-3, I-oo′-4, I-oo′-5, I-oo′-6, I-oo′-7, I-oo′-8, I-oo′-9, I-oo′-10, I-oo″-1, I-oo″-2, I-oo″-3, I-oo″-4, I-oo″-5, I-oo″-6, I-oo″-7, I-oo″-8, I-oo″-9, or I-oo″-10, or a pharmaceutically acceptable salt thereof, wherein:

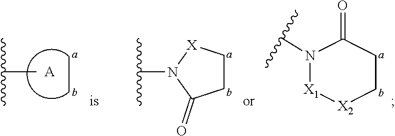

Y is a bond, Y₁, O, NH, NR₂, C(O)O, OC(O), C(O)NR₂′, NR₂′C(O), Y₁—O, Y₁—NH, Y₁—NR₂, Y₁—C(O), Y₁—C(O)O, Y₁—OC(O), Y₁—C(O)NR₂′, or Y₁—NR₂′C(O), wherein Y₁ is C₁-C₆ alkylene, C₂-C₆ alkenylene, or C₂-C₆ alkynylene;
X is C(O) or C(R³)₂;
X₁—X₂ is C(R₃)═N or C(R³)₂—C(R³)₂;
each R₁ is independently halogen, nitro, NH₂, OH, C(O)OH, C₁-C₆ alkyl, or C₁-C₆ alkoxy;
R₂ is C₁-C₆ alkyl, C₂-C₆ alkenyl, C₃-C₈ cycloalkyl, 3- to 8-membered heterocycloalkyl, C(O)—C₁-C₆ alkyl, C(O)—C₂-C₆ alkenyl, C(O)—C₃-C₈ cycloalkyl, or C(O)-3- to 8-membered heterocycloalkyl, and R₂ is optionally substituted with one or more of halogen, N(Rₐ)₂, NHC(O)Rₐ, NHC(O)ORₐ, OR_{b}, C₃-C₈ cycloalkyl, 3- to 8-membered heterocycloalkyl, C₆-C₁₀ aryl, or 5- to 10-membered heteroaryl, wherein each of the C₃-C₈ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
R₂′ is H, C₁-C₆ alkyl, C₂-C₆ alkenyl, C₃-C₅ cycloalkyl, or 3- to 8-membered heterocycloalkyl, and R₂′, when not being H, is optionally substituted with one or more of halogen, N(Rₐ)₂, NHC(O)Rₐ, NHC(O)ORₐ, OR_{b}, C₃-C₅ cycloalkyl, 3- to 8-membered heterocycloalkyl, C₆-C₁₀ aryl, or 5- to 10-membered heteroaryl, wherein each of the C₃-C₅ cycloalkyl, 3- to 8-membered heterocycloalkyl, C₆-C₁₀ aryl or 5- to 10-membered heteroaryl is optionally further substituted with one or more of halogen, NH₂, CN, nitro, OH, C(O)OH, C₁-C₆ alkyl, C₁-C₆ haloalkyl, C₁-C₆ alkoxy, or C₁-C₆ haloalkoxy;
each R₃ is independently H or C₁-C₃ alkyl optionally substituted with C₆-C₁₀ aryl or 5- to 10-membered heteroaryl;
each R₃′ is independently C₁-C₃ alkyl;
each R₄ is independently H or C₁-C₃ alkyl; or two R₄, together with the carbon atom to which they are attached, form C(O), a C₃-C₆ carbocycle, or a 4-, 5-, or 6-membered heterocycle comprising 1 or 2 heteroatoms selected from N and O;
R₅ is H, C₁-C₃ alkyl, F, or Cl;
each Rₐ independently is H or C₁-C₆ alkyl;
R_{b} is H or tosyl;
t is 0 or 1;
m is 0, 1, 2 or 3; and
n is 0, 1 or 2.

In certain embodiments, the present invention provides a compound of Formula I, wherein DIM (e.g., LBM) is an E3 ubiquitin ligase (cereblon) binding moiety thereby forming a compound of formula I-pp-1, I-pp-2, I-pp-3, I-pp-4, I-pp-5, or I-pp-6 respectively:

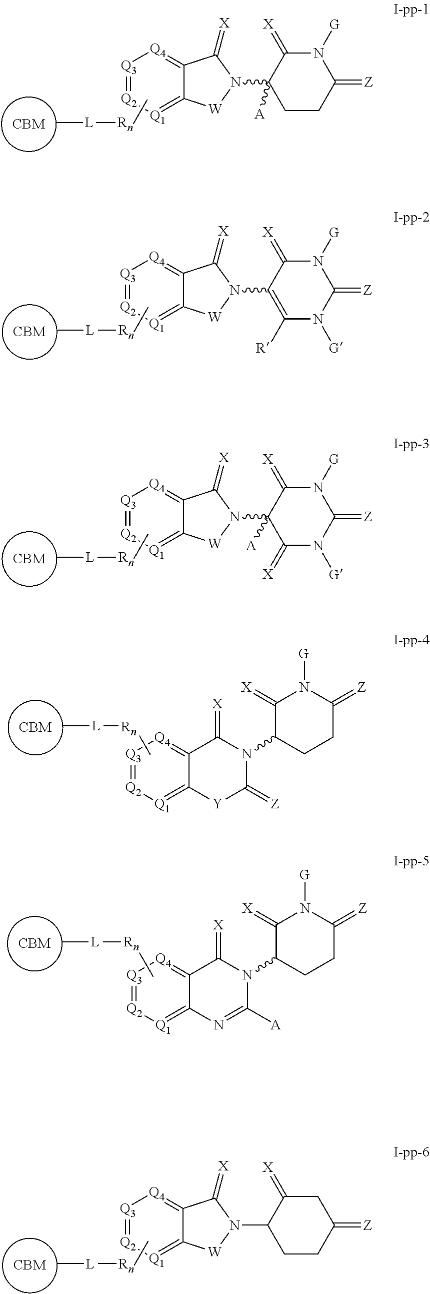

or a pharmaceutically acceptable salt thereof, wherein L and CBM are as defined above and described in [symbol] embodiments herein, and wherein each of the variables A, G, G′, Q₁, Q₂, Q₃, Q₄, R, R′, W, X, Y, Z, [symbol] and n is as defined and described in WO 2016/197114 and US 2018/0147202, the entirety of each of which is herein incorporated by reference.

In some embodiments, LBM is

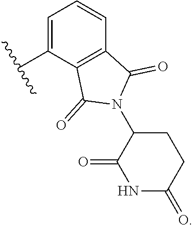 [SMILES: *c1cccc2c1C(=O)N(C1CCC(=O)NC1=O)C2=O]

In some embodiments, LBM is

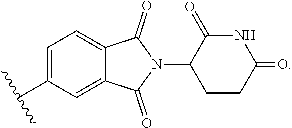 [SMILES: *c1ccc2c(c1)C(=O)N(C1CCC(=O)NC1=O)C2=O]

In some embodiments, LBM is

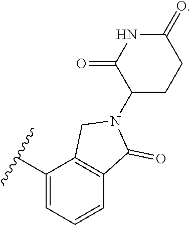 [SMILES: *c1cccc2c1CN(C1CCC(=O)NC1=O)C2=O]

In some embodiments, LBM is

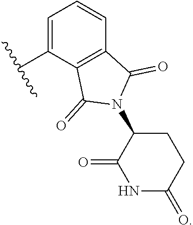 [SMILES: *c1cccc2c1C(=O)N([C@H]1CCC(=O)NC1=O)C2=O]

In some embodiments, LBM is

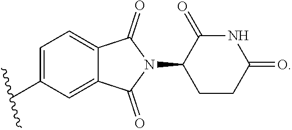 [SMILES: *c1ccc2c(c1)C(=O)N([C@@H]1CCC(=O)NC1=O)C2=O]

In some embodiments, LBM is

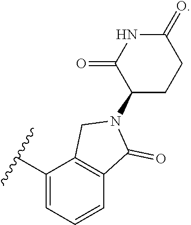 [SMILES: *c1cccc2c1CN([C@@H]1CCC(=O)NC1=O)C2=O]

In some embodiments, LBM is

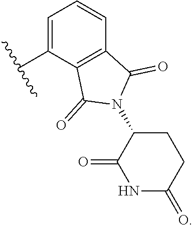 [SMILES: *c1cccc2c1C(=O)N([C@@H]1CCC(=O)NC1=O)C2=O]

In some embodiments, LBM is

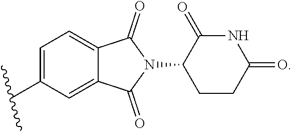 [SMILES: *c1ccc2c(c1)C(=O)N([C@H]1CCC(=O)NC1=O)C2=O]

In some embodiments, LBM is

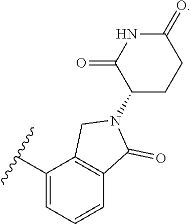 [SMILES: *c1cccc2c1CN([C@H]1CCC(=O)NC1=O)C2=O]

In some embodiments, LBM is selected from those in Table 1 below.

In certain embodiments, the present invention provides a compound of Formula I, wherein DIM (e.g., LBM) is an E3 ubiquitin ligase (cereblon) binding moiety thereby forming a compound of formula I-qq-1, I-qq-2, or I-qq-3 respectively:

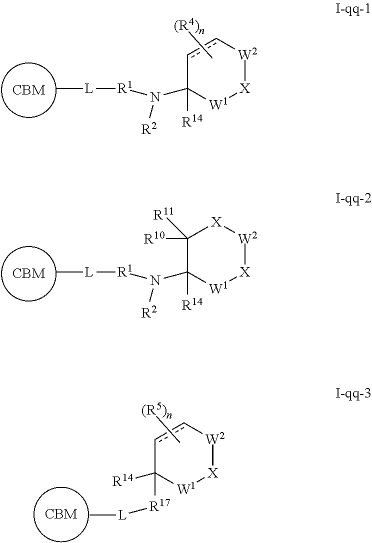

or a pharmaceutically acceptable salt thereof, wherein L and CBM are as defined above and described herein, and wherein each of the variables R¹, R², R⁴, R⁵, R¹⁰, R¹¹, R¹⁴, R¹⁷, W¹, W², X, [symbol] and n is as defined in WO 2017/197051 which is herein incorporated by reference in its entirety and wherein

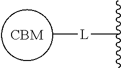

is attached to R¹, the ring formed by combining R¹ and R², or R¹⁷ at the site of attachment of R¹² as defined in WO 2017/197051 such that

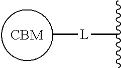

takes the place of the R¹² substituent.

In some embodiments, the present invention provides a compound of formula I, wherein DIM (e.g., LBM) is an E3 ubiquitin ligase (cereblon) binding moiety thereby forming a compound of formula I-rr-1, I-rr-2, I-rr-3, or I-rr-4, respectively:

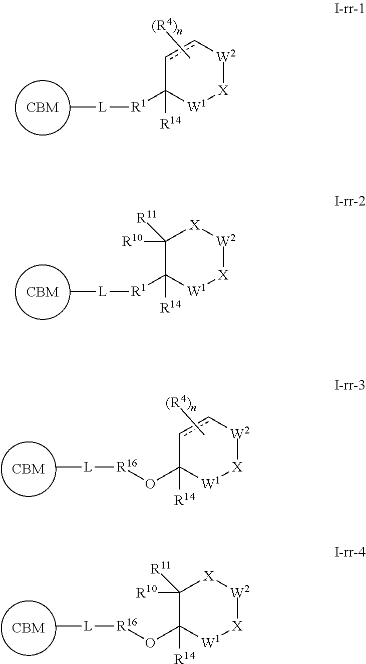

or a pharmaceutically acceptable salt thereof, wherein L and CBM are as defined above and described herein, and wherein each of the variables R¹, R⁴, R¹⁰, R¹¹, R¹⁴, R¹⁶, W¹, W², X, [symbol] and n is as defined in WO 2018/237026, the entirety of each of which is herein incorporated by reference, and wherein

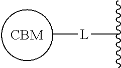

is attached to R¹ or R¹⁶ at the site of attachment of R¹² as defined in WO 2018/237026, such that

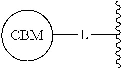

takes the place of the R¹² substituent.

In some embodiments, the present invention provides a compound of formula I, wherein DIM (e.g., LBM) is an E3 ubiquitin ligase (cereblon) binding moiety thereby forming a compound of formula I-ss-1 or I-ss-3, respectively:

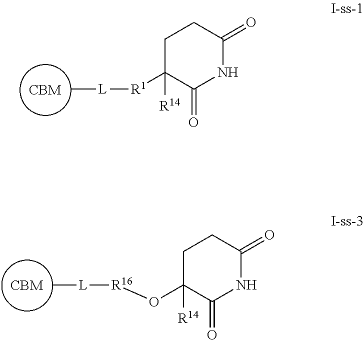

or a pharmaceutically acceptable salt thereof, wherein L and CBM are as defined above and described herein, and wherein each of the variables R¹, R¹⁴, and R¹⁶ is as defined in WO 2018/237026, the entirety of each of which is herein incorporated by reference, and wherein

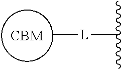

is attached to R¹ or R¹⁶ at the site of attachment of R¹² as defined in WO 2018/237026, such that

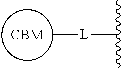

takes the place of the R¹² substituent.

In certain embodiments, the present invention provides a compound of formula I, wherein DIM (e.g., LBM) is an E3 ubiquitin ligase (cereblon) binding moiety thereby forming a compound of formula I-tt-1, I-tt-2, I-tt-3, I-tt-4, I-tt-5, I-tt-6, I-tt-7, or I-tt-8.

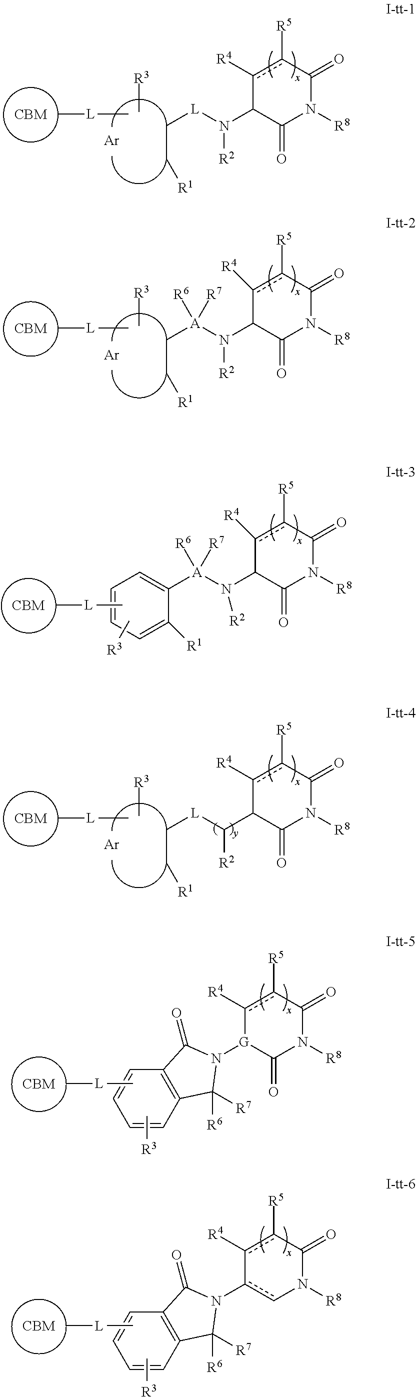

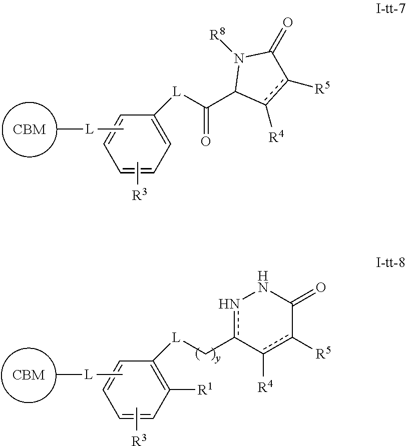

or a pharmaceutically acceptable salt thereof, wherein L and CBM are as defined above and described in embodiments herein, and wherein each of the variables Ar, R¹, R², R³, R⁴, R⁵, R⁶, R⁷, R⁸, A, L, x, y, and [symbol] is as described and defined in WO 2017/161119, the entirety of each of which is herein incorporated by [symbol] reference.

In certain embodiments, the present invention provides a compound of formula I, wherein DIM (e.g., LBM) is an E3 ubiquitin ligase (cereblon) binding moiety thereby forming a compound of formula I-uu:

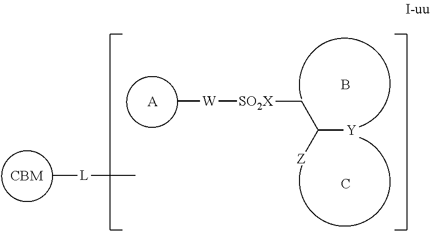

or a pharmaceutically acceptable salt thereof, wherein L and CBM are as defined above and described in embodiments herein, and wherein each of the variables A, B, C, W, X, Y, and Z is as described and defined in U.S. Pat. No. 5,721,246, the entirety of each of which is herein incorporated by reference.

In certain embodiments, the present invention provides a compound of formula I, wherein DIM (e.g., LBM) is an E3 ubiquitin ligase (cereblon) binding moiety thereby forming a compound of formula I-vv:

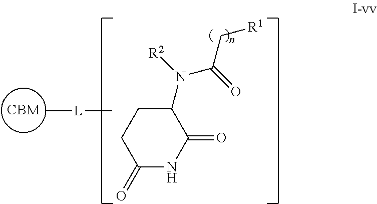

or a pharmaceutically acceptable salt thereof, wherein L and CBM are as defined above and described in embodiments herein, and wherein each of the variables R₁, R₂, and n is as described and defined in WO 2019/043214, the entirety of each of which is herein incorporated by reference.

In some embodiments, LBM is a IAP E3 Ubiquitin ligase binding moiety recited in Varfolomeev, E. et al., IAP Antagonists Induce Autoubiquitination of c-IAPs, NF-κB activation, and TNFα-Dependent Apoptosis, Cell, 2007, 131(4): 669-81, such as, for example:

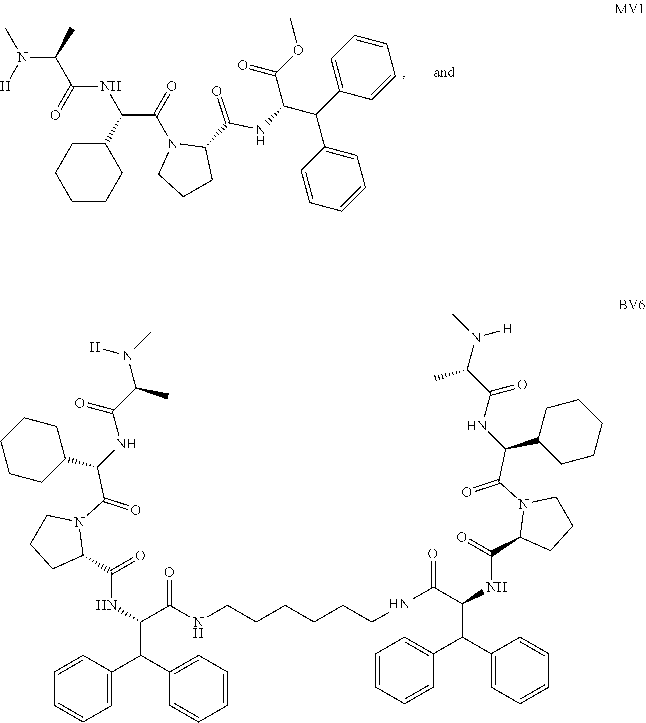 [SMILES: CN[C@@H](C)C(=O)N[C@H](C(=O)N1CCC[C@H]1C(=O)N[C@H](C(=O)OC)C(c1ccccc1)c1ccccc1)C1CCCCC1 ; CN[C@@H](C)C(=O)N[C@H](C(=O)N1CCC[C@H]1C(=O)N[C@H](C(=O)NCCCCCCNC(=O)[C@@H](NC(=O)[C@@H]1CCCN1C(=O)[C@@H](NC(=O)[C@H](C)NC)C1CCCCC1)C(c1ccccc1)c1ccccc1)C(c1ccccc1)c1ccccc1)C1CCCCC1]

wherein

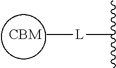

is attached to a modifiable carbon, oxygen, nitrogen or sulfur atom.

In certain embodiments, the present invention provides a compound of Formula I, wherein DIM (e.g., LBM) is a VHL E3 ubiquitin ligase binding moiety thereby forming a compound of formula I-ww-1, I-ww-2, I-ww-3, I-ww-4, or I-ww-5 respectively:

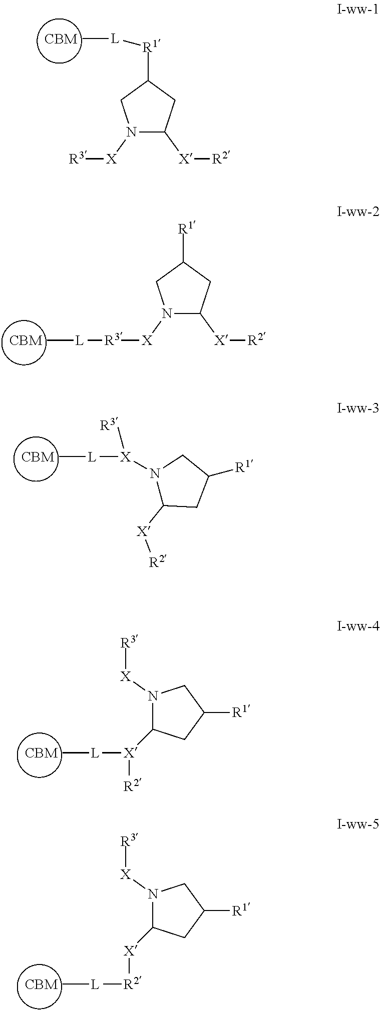

or a pharmaceutically acceptable salt thereof, wherein L and CBM are as defined above and described in embodiments herein, and wherein each of the variables R^{1′}, R^{2′}, R^{3′}, X, and X′ is as defined and described in WO 2013/106643 and US 2014/0356322, the entirety of each of which is herein incorporated by reference.

In certain embodiments, the present invention provides a compound of Formula I, wherein DIM (e.g., LBM) is a VHL E3 ubiquitin ligase binding moiety thereby forming a compound of formula I-xx-1, I-xx-2, I-xx-3, I-xx-4, I-xx-5 or I-xx-6 respectively:

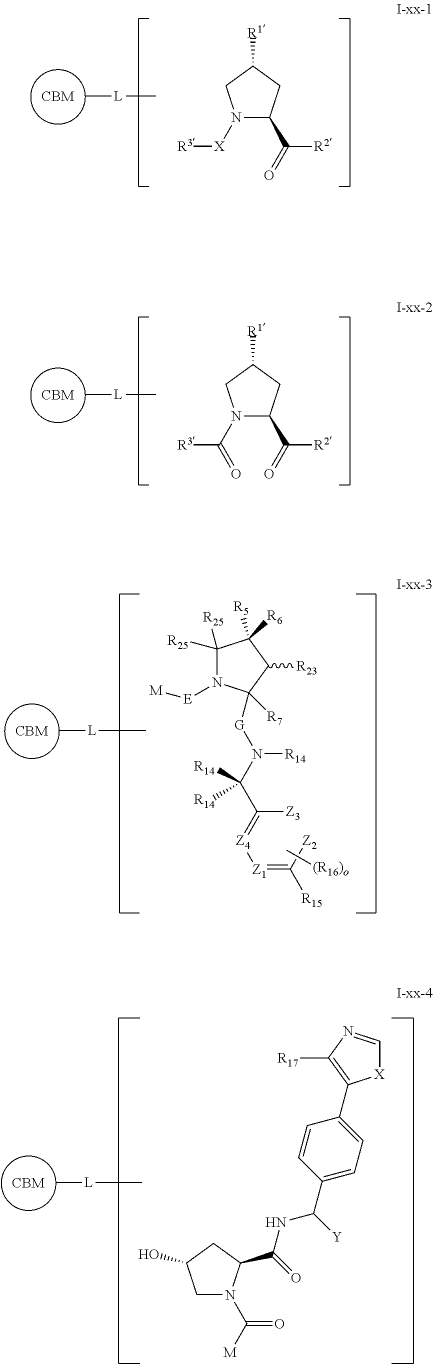

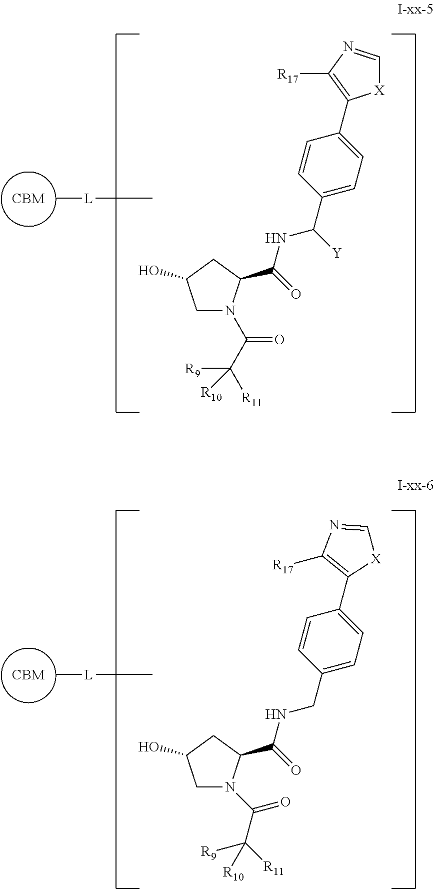

or a pharmaceutically acceptable salt thereof, wherein L and CBM are as defined above and described in embodiments herein, and wherein each of the variables R^{1′}, R^{2′}, R^{3′}, R₅, R₆, R₇, R₉, R₁₀, R₁₁, R₁₄, R₁₅, R₁₆, R₁₇, R₂₃, R₂₅, E, G, M, X, X′, Y, Z₁, Z₂, Z₃, Z₄, and o is as defined and described in WO 2016/149668 and US 2016/0272639, the entirety of each of which is herein incorporated by reference.

As used herein, depiction of brackets around any LBM

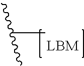

means that the

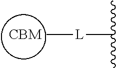

moiety is covalently attached to said LBM at any available modifiable carbon, nitrogen, oxygen, or sulfur atom. For purposes of clarity and by way of example, such available modifiable carbon, nitrogen, oxygen, or sulfur atoms in the following LBM compound structure are depicted below, wherein each wavy bond defines the point of attachment to said

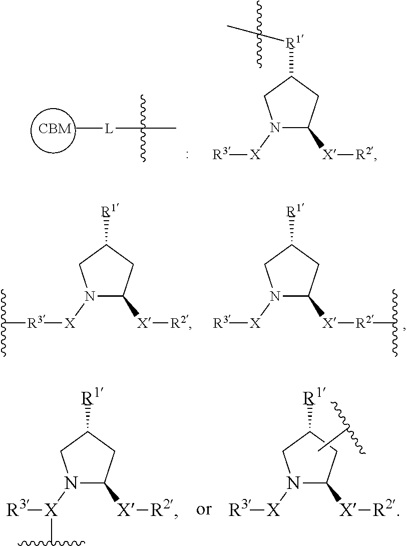

In certain embodiments, the present invention provides a compound of Formula I, wherein DIM (e.g., LBM) is a VHL E3 ubiquitin ligase binding moiety thereby forming a compound of formula I-yy-1, I-yy-2, or I-yy-3 respectively:

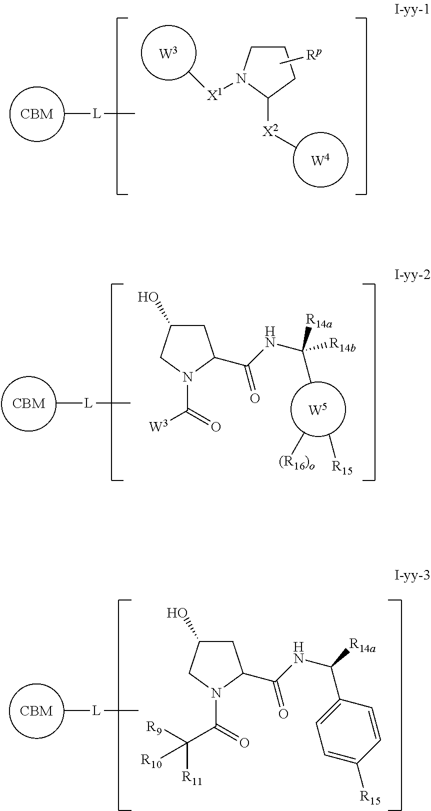

or a pharmaceutically acceptable salt thereof, wherein L and CBM are as defined above and described in embodiments herein, and wherein each of the variables R^{p}, R₉, R₁₀, R₁₁, R₁₄ₐ, R_{14b}, R₁₅, R₁₆, W³, W⁴, W⁵, X¹, X², and o is as defined and described in WO 2016/118666 and US 2016/0214972, the entirety of each of which is herein incorporated by reference.

In certain embodiments, the present invention provides a compound of Formula I, wherein DIM (e.g., LBM) is a CRBN or VHL E3 ubiquitin ligase binding moiety thereby forming a compound of formula I-zz-1, I-zz-2, I-zz-3, I-zz-4, I-zz-5, I-zz-6, or I-zz-7 respectively:

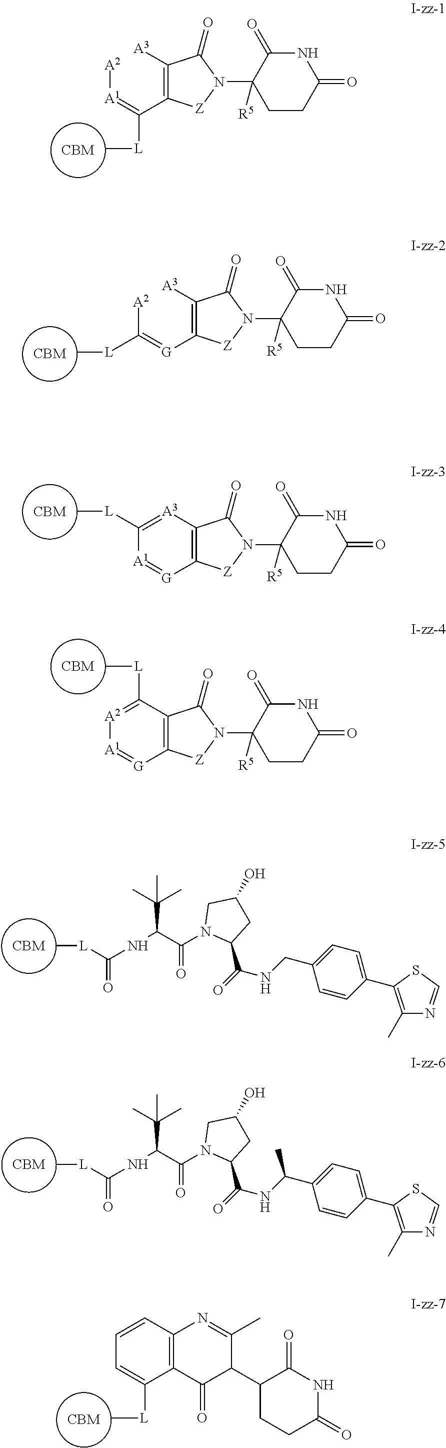

or a pharmaceutically acceptable salt thereof, wherein L and CBM are as defined above and described in embodiments herein, and wherein each of the variables A¹, A², A³, R⁵, G and Z is as defined and described in WO 2017/176958.

In certain embodiments, the present invention provides a compound of Formula I, wherein DIM (e.g., LBM) is a CRBN E3 ubiquitin ligase binding moiety thereby forming a compound of formula I-zz′-1, I-zz″-1, I-zz′-2, I-zz′-2, I-zz′-3, I-zz″-3, I-zz′-4, I-zz″-4, I-zz′-7 or I-zz″-7 respectively:

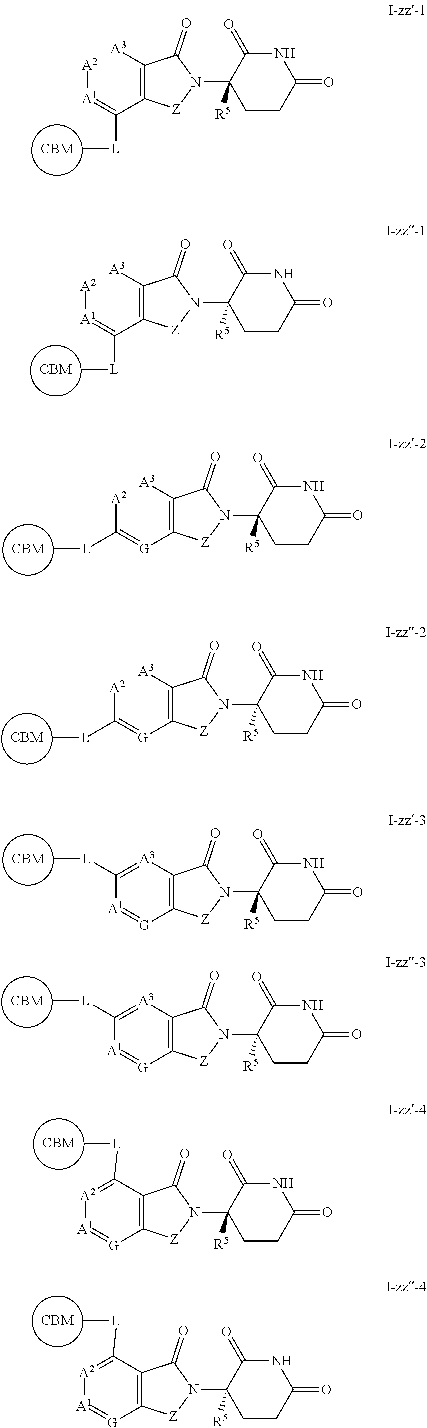

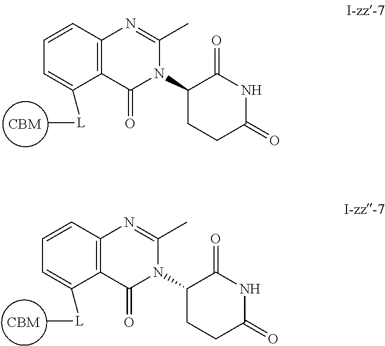

or a pharmaceutically acceptable salt thereof, wherein L and CBM are as defined above and described in embodiments herein, and wherein each of the variables A¹, A², A³, R⁵, G and Z is as defined and described in WO 2017/176958, the entirety of which is herein incorporated by reference.

In certain embodiments, the present invention provides a compound of Formula I, wherein DIM (e.g., LBM) is a MDM2 (i.e. human double minute 2 or HDM2) E3 ligase binding moiety thereby forming a compound of formula I-aaa-1, I-aaa-2, I-aaa-3, I-aaa-4, I-aaa-5, I-aaa-6, I-aaa-7, I-aaa-8, I-aaa-9, I-aaa-10, I-aaa-11, I-aaa-12, I-aaa-13, I-aaa-14, I-aaa-15, I-aaa-16, I-aaa-17, or I-aaa-18 respectively:

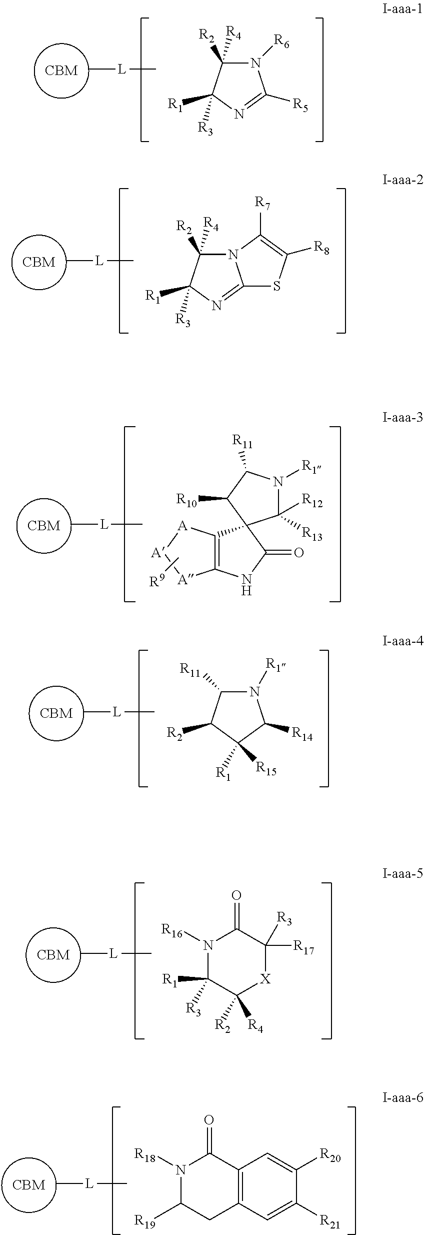

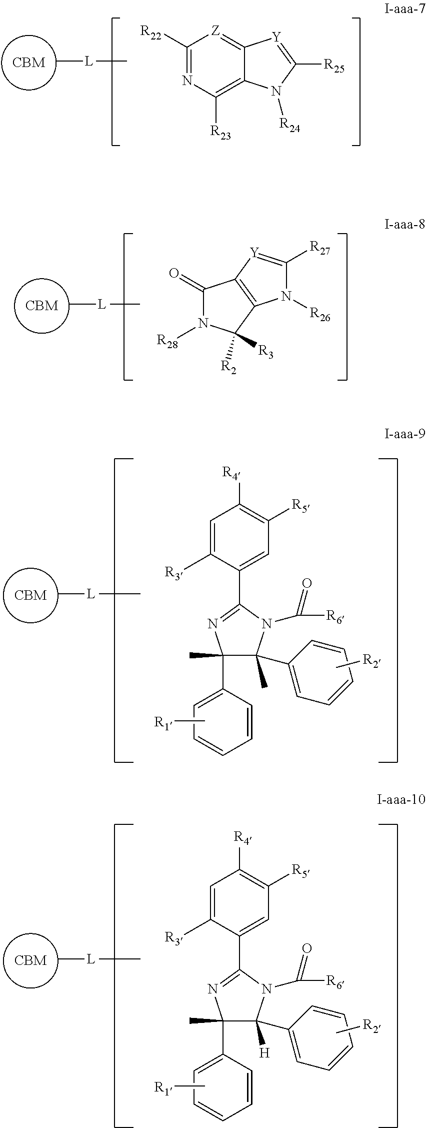

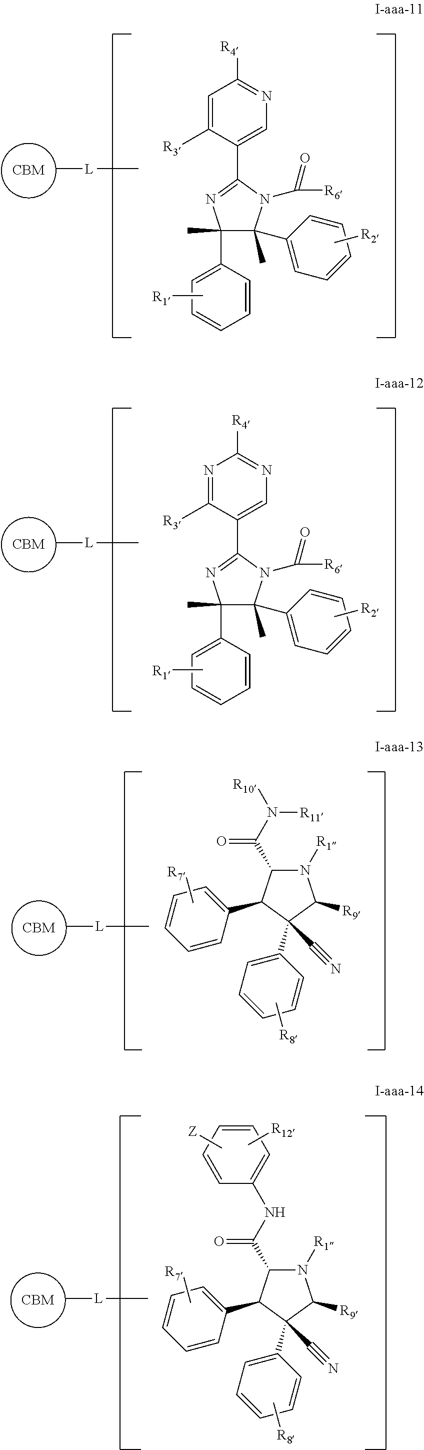

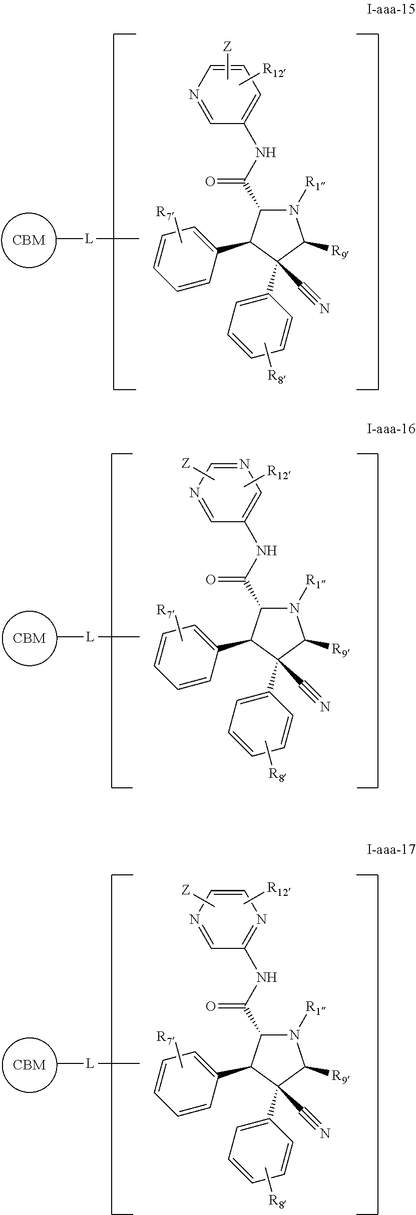

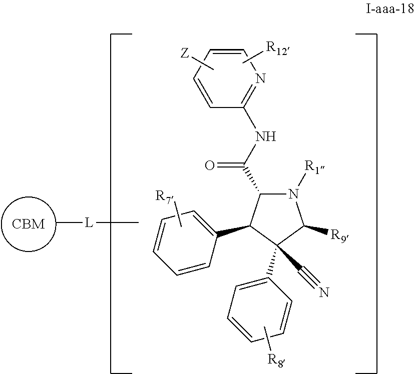

or a pharmaceutically acceptable salt thereof, wherein L and CBM are as defined above and described in embodiments herein, and wherein each of the variables R₁, R₂, R₃, R⁴, R₅, R₆, R₇, R₈, R₉, R₁₀, R₁₁, R¹², R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R_{1′}, R_{2′}, R_{3′}, R_{4′}, R_{5′}, R_{6′}, R_{7′}, R_{8′}, R₉, R_{10′}, R_{11′}, R_{12′}, R_{1″}, A, A′, A″, X, Y, and Z is as defined and described in WO 2017/011371 and US 2017/0008904, the entirety of each of which is herein incorporated by reference.

In certain embodiments, the present invention provides a compound of Formula I, wherein DIM (e.g., LBM) is a MDM2 (i.e. human double minute 2 or HDM2) E3 ligase binding moiety thereby forming a compound of formula I-aaa-19, I-aaa-20, or I-aaa-21 respectively

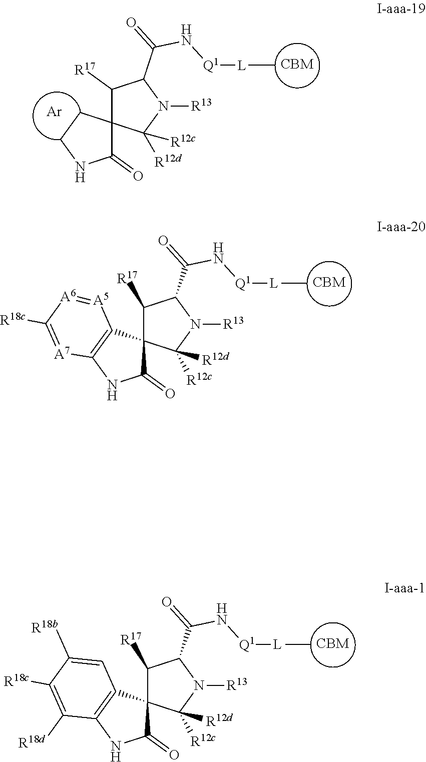

or a pharmaceutically acceptable salt thereof, wherein each of the variables R^{12c}, R^{12d}, R¹³, R¹⁷, R^{18b}, R^{18c}, R^{18a}, A⁵, A⁶, A⁷, Q¹, and Ar is as defined and described in WO 2017/176957 and US2019/127387.

In certain embodiments, the present invention provides a compound of Formula I, wherein DIM (e.g., LBM) is an IAP E3 ubiquitin ligase binding moiety thereby forming a compound of formula I-bbb-1, I-bbb-2, I-bbb-3, or I-bbb-4 respectively:

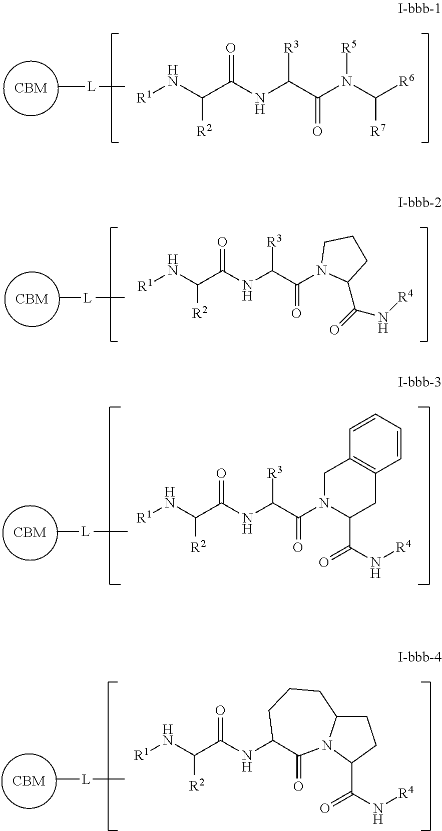

or a pharmaceutically acceptable salt thereof, wherein L and CBM are as defined above and described in embodiments herein, and wherein each of the variables R¹, R², R³, R⁴, R⁵, R⁶, and R⁷, is as defined and described in WO 2017/011590 and US 2017/0037004, the entirety of each of which is herein incorporated by reference.

In certain embodiments, the present invention provides a compound of Formula I, wherein DIM (e.g., LBM) is an E3 ubiquitin ligase (cereblon) binding moiety, a DCAF 15 E3 ubiquitin ligase binding moiety, or a VHL E3 ubiquitin ligase binding moiety; thereby forming a compound of formula I-ccc-1, I-ccc-2, or I-ccc-3:

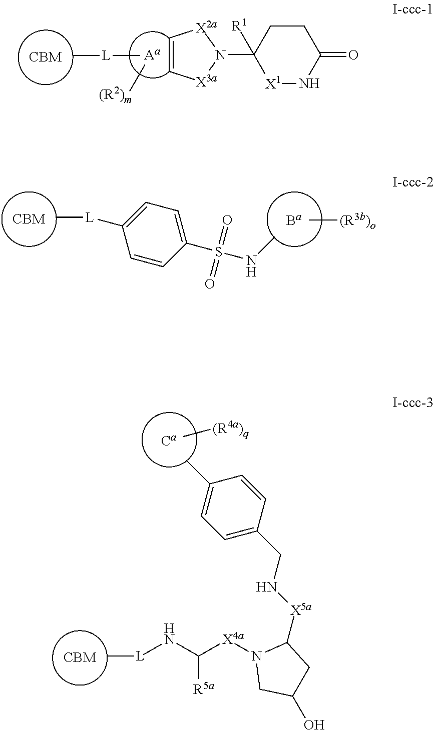

or a pharmaceutically acceptable salt thereof, wherein L and CBM is as defined above and described in embodiments herein, and wherein:
each of X¹, X^{2a}, and X^{3a} is independently a bivalent moiety selected from a covalent bond, —CH₂—, —C(O)—, —C(S)—, or

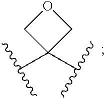 [SMILES: *C1(*)COC1]

each of X^{4a} and X^{5a} is independently a bivalent moiety selected from —CH₂—, —C(O)—, —C(S)—, or

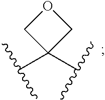 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R^{5a} is hydrogen or C₁₋₆ aliphatic;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A^{a} is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
Ring B^{a} is selected from 6-membered aryl containing 0-2 nitrogen atoms or a 8-10 membered bicyclic heteroaryl having 1-5 heteroatoms independently selected from nitrogen, oxygen, or sulfur;
Ring C^{a} is a selected from 6-membered aryl containing 0-2 nitrogen atoms or a 5-membered heteroaryl with 1-3 heteroatoms independently selected from nitrogen, oxygen or sulfur;
m is 0, 1, 2, 3 or 4;
o is 0, 1, 2, 3 or 4;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I-ccc-1, wherein DIM (e.g., LBM) is an E3 ubiquitin ligase (cereblon) binding moiety thereby forming a compound of formula I-ccc′-1 or I-ccc″-1:

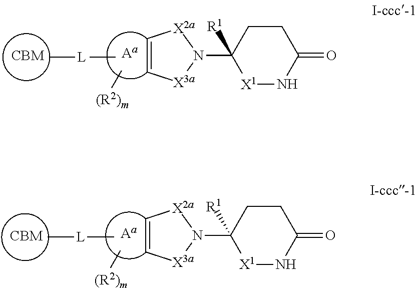

or a pharmaceutically acceptable salt thereof, wherein CBM, L, Ring Aa, X¹, X^{2a}, X^{3a}, R¹, R² and m are as described above.

As defined above and described herein, each of X¹, X^{2a}, and X^{3a} is independently a bivalent moiety selected from a covalent bond, —CH₂—, —C(O)—, —C(S)—, or

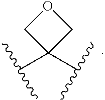 [SMILES: *C1(*)COC1]

In some embodiments, X¹ is a covalent bond, —CH₂—, —C(O)—, —C(S)—, or

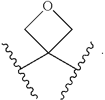 [SMILES: *C1(*)COC1]

In some embodiments, X¹ is selected from those depicted in Table 1, below.

In some embodiments, X^{2a} is a covalent bond, —CH₂—, —C(O)—, —C(S)—, or

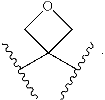 [SMILES: *C1(*)COC1]

In some embodiments, X^{2a} is selected from those depicted in Table 1, below.

In some embodiments, X^{3a} is a covalent bond, —CH₂—, —C(O)—, —C(S)—, or

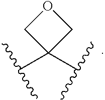 [SMILES: *C1(*)COC1]

In some embodiments, X^{3a} is selected from those depicted in Table 1, below.

As defined above and described herein, each of X⁴ and X⁵ is independently a bivalent moiety selected from —CH₂—, —C(O)—, —C(S)—, or

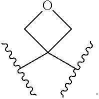 [SMILES: *C1(*)COC1]

In some embodiments, X^{4a} is —CH₂—, —C(O)—, —C(S)—, or

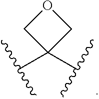 [SMILES: *C1(*)COC1]

In some embodiments, X^{4a} is selected from those depicted in Table 1, below.

In some embodiments, X^{5a} is —CH₂—, —C(O)—, —C(S)—, or

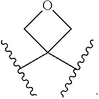 [SMILES: *C1(*)COC1]

In some embodiments, X^{5a} is selected from those depicted in Table 1, below.

As defined above and described herein, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is selected from those depicted in Table 1, below.

As defined above and described herein, each of R², R^{3b}, and R^{4a} is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is hydrogen, —R, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is selected from those depicted in Table 1, below.

In some embodiments, R^{3b} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{3b} is methyl.

In some embodiments, R^{3b} is selected from those depicted in Table 1, below.

In some embodiments, R^{4a} is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R^{4a} is methyl.

In some embodiments, R^{4a} is selected from those depicted in Table 1, below.

As defined above and described herein, R^{a} is hydrogen or C₁₋₆ aliphatic.

In some embodiments, R^{1a} is t-butyl.

In some embodiments, R^{1a} is selected from those depicted in Table 1, below.

As defined above and described herein, each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁶ is an optionally substituted C₁₋₆ aliphatic group. In some embodiments, R⁶ is an optionally substituted phenyl. In some embodiments, R⁶ is an optionally substituted 4-7 membered saturated or partially unsaturated heterocyclic ring having 1-2 heteroatoms independently selected from nitrogen, oxygen, and sulfur. In some embodiments, R⁶ is an optionally substituted 5-6 membered heteroaryl ring having 1-4 heteroatoms independently selected from nitrogen, oxygen, and sulfur.

In some embodiments, R⁶ is selected from those depicted in Table 1, below.

As defined above and described herein, Ring Aa is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur.

In some embodiments Ring Aa is a fused 6-membered aryl containing 0-2 nitrogen atoms. In some embodiments Ring Aa is a fused 5 to 7-membered partially saturated carbocyclyl. In some embodiments Ring Aa is a fused 5 to 7-membered partially saturated heterocyclyl with 1-2 heteroatoms independently selected from nitrogen, oxygen or sulfur. In some embodiments Ring Aa is a fused 5-membered heteroaryl with 1-3 heteroatoms independently selected from nitrogen, oxygen or sulfur.

In some embodiments, Ring Aa is a fused phenyl.

In some embodiments, Ring Aa is selected from those depicted in Table 1, below.

As defined above and described herein, Ring B^{a} is selected from 6-membered aryl containing 0-2 nitrogen atoms or a 8-10 membered bicyclic heteroaryl having 1-5 heteroatoms independently selected from nitrogen, oxygen, or sulfur.

In some embodiments, Ring B^{a} is a 6-membered aryl containing 0-2 nitrogen atoms. In some embodiments, Ring B^{a} is a 8-10 membered bicyclic heteroaryl having 1-5 heteroatoms independently selected from nitrogen, oxygen, or sulfur.

In some embodiments, Ring B^{a} is

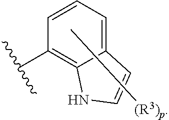

In some embodiments, Ring B^{a} is selected from those depicted in Table 1, below.

As defined above and described herein, Ring C^{a} is selected from 6-membered aryl containing 0-2 nitrogen atoms or a 5-membered heteroaryl with 1-3 heteroatoms independently selected from nitrogen, oxygen or sulfur.

In some embodiments, Ring C^{a} is a 6-membered aryl containing 0-2 nitrogen atoms. In some embodiments, Ring C^{a} is a 5-membered heteroaryl with 1-3 heteroatoms independently selected from nitrogen, oxygen or sulfur.

In some embodiments, Ring C^{a} is

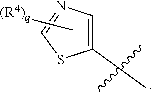

In some embodiments, Ring C^{a} is selected from those depicted in Table 1, below.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In some embodiments, m is selected from those depicted in Table 1, below.

In some embodiments, o is selected from those depicted in Table 1, below.

As defined above and described herein, o is 0, 1, 2, 3 or 4.

In some embodiments, o is 0. In some embodiments, o is 1. In some embodiments, o is 2. In some embodiments, o is 3. In some embodiments, o is 4.

In some embodiments, o is selected from those depicted in Table 1, below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1, below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is phenyl. In some embodiments, R is a 4-7 membered saturated or partially unsaturated heterocyclic having 1-2 heteroatoms independently selected from nitrogen, oxygen, and sulfur. In some embodiments, R is a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is selected from those depicted in Table 1, below.

In certain embodiments, the present invention provides a compound of formula I, wherein DIM (e.g., LBM) is a VHL binding moiety thereby forming a compound of formula I-ddd:

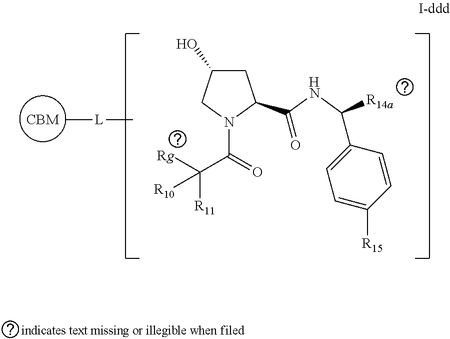

or a pharmaceutically acceptable salt thereof, wherein L and CBM are as defined above and described in embodiments herein, and wherein each of the variables R₉, R₁₀, R₁₁, R₁₄ₐ, and R₁₅ is as described and defined in WO 2017/030814, WO 2016/118666, and US 2017/0327469, the entirety of each of which is herein incorporated by reference.

In certain embodiments, the present invention provides a compound of formula I, wherein DIM (e.g., LBM) is a VHL binding moiety thereby forming a compound of formula I-eee-1 or I-eee-2:

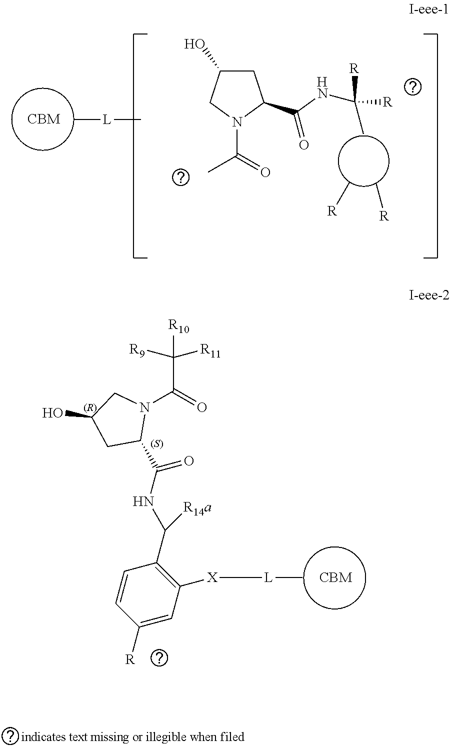

or a pharmaceutically acceptable salt thereof, wherein L and CBM are as defined above and described in embodiments herein, and wherein each of the variables X, W, R₉, R₁₀, R₁₁, R₁₄ₐ, and R_{14b}, R₁₅, R¹⁶, and o is as described and defined in WO 2017/030814, WO 2016/118666, and US 2017/0327469, the entirety of each of which is herein incorporated by reference.

In certain embodiments, the present invention provides a compound of formula I, wherein DIM (e.g., LBM) is an IAP binding moiety thereby forming a compound of formula I-fff:

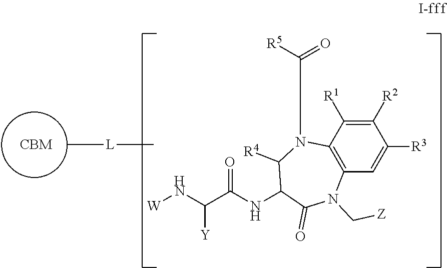

or a pharmaceutically acceptable salt thereof, wherein L and CBM are as defined above and described in embodiments herein, and wherein each of the variables W, Y, Z, R¹, R², R³, R⁴, and R is as described and defined in WO 2014/044622, US 2015/0225449. WO 2015/071393, and US 2016/0272596, the entirety of each of which is herein incorporated by reference.

In certain embodiments, the present invention provides a compound of formula I, wherein DIM (e.g., LBM) is a MDM2 binding moiety thereby forming a compound of formula I-ggg:

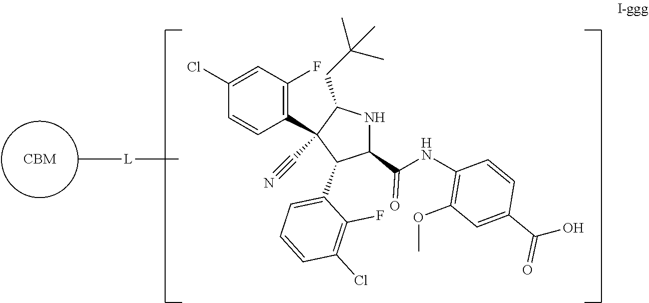

or a pharmaceutically acceptable salt thereof, wherein L and CBM are as defined above and described in embodiments herein, as described and defined in Hines, J. et al., Cancer Res. (DOI: 10.1158/0008-5472.CAN-18-2918), the entirety of each of which is herein incorporated by reference.

In certain embodiments, the present invention provides a compound of formula I, wherein DIM (e.g., LBM) is a DCAF16 binding moiety thereby forming a compound of formula I-hhh:

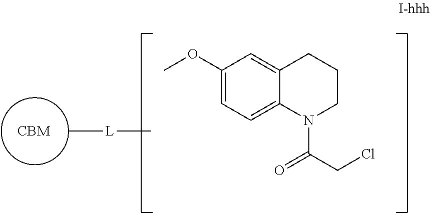

or a pharmaceutically acceptable salt thereof, wherein L and CBM are as defined above and described in embodiments herein, as described and defined in Zhang, X. et al., bioRxiv (doi: https://doi.org/10.1101/443804), the entirety of each of which is herein incorporated by reference.

In certain embodiments, the present invention provides a compound of formula I, wherein DIM (e.g., LBM) is a RNF114 binding moiety thereby forming a compound of formula 1-iii:

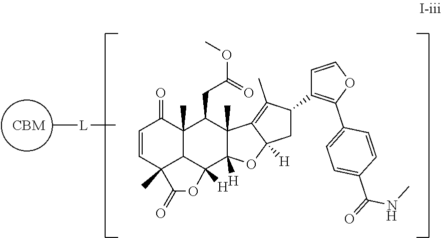

or a pharmaceutically acceptable salt thereof, wherein L and CBM are as defined above and described in embodiments herein, as described and defined in Spradin, J. N. et al., bioRxiv (doi: https://doi.org/10.1101/436998), the entirety of each of which is herein incorporated by reference.

In certain embodiments, the present invention provides a compound of formula I, wherein DIM (e.g., LBM) is a RNF4 binding moiety thereby forming a compound of formula I-jjj:

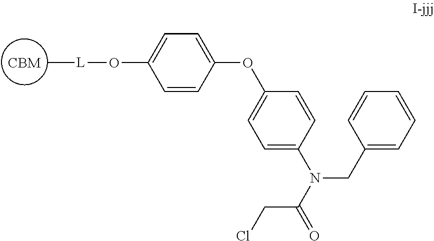

or a pharmaceutically acceptable salt thereof, wherein L and CBM are as defined above and described in embodiments herein, as described and defined in Ward, C. C., et al., bioRxiv (doi: https://doi.org/10.1101/439125), the entirety of each of which is herein incorporated by reference.

In certain embodiments, the present invention provides a compound of formula I, wherein DIM (e.g., LBM) is a VHL binding moiety thereby forming a compound of formula I-nnn-1 or I-nnn-2:

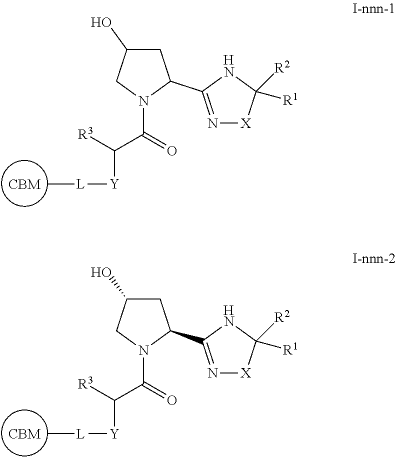

or a pharmaceutically acceptable salt thereof, wherein L and CBM are as defined above and described in embodiments herein, and wherein each of the variables R¹, R², R³, X, and Y is as defined and described in WO 2019/084026, the entirety of each of which is herein incorporated by reference.

In certain embodiments, the present invention provides a compound of formula I, wherein DIM (e.g., LBM) is a VHL binding moiety thereby forming a compound of formula I-ooo-1 or I-ooo-2:

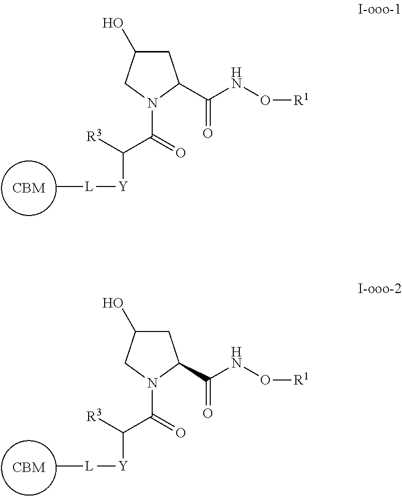

or a pharmaceutically acceptable salt thereof, wherein L and CBM are as defined above and described in embodiments herein, and wherein each of the variables R¹, R³, and Y is as defined and described in WO 2019/084030, the entirety of each of which is herein incorporated by reference.

In certain embodiments, the present invention provides a compound of formula I, wherein DIM (e.g., LBM) is a E3 ubiquitin ligase (cereblon) binding moiety thereby forming a compound of formula I-ppp-1, I-ppp-2, I-ppp-3, or I-ppp-4:

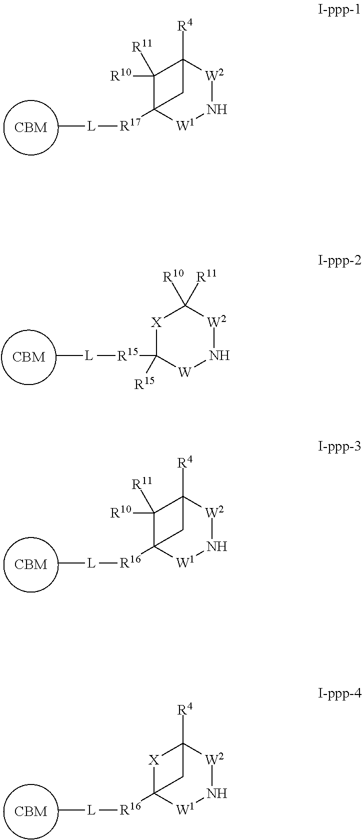

or a pharmaceutically acceptable salt thereof, wherein L and CBM are as defined above and described herein, and wherein each of the variables R⁴, R¹⁰, R¹¹, R¹⁵, R⁶, R¹⁷, W¹, W², and X is as defined in WO 2019/099868 which is herein incorporated by reference in its entirety, and wherein

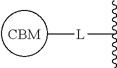

is attached to R¹⁷ or R¹⁶ at the site of attachment of R¹² as defined in WO 2018/237026, such that

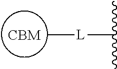

takes the place of the R¹² substituent.

In some embodiments, LBM is

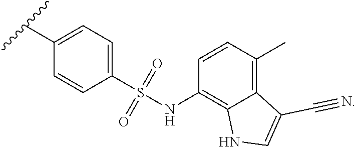 [SMILES: *c1ccc(S(=O)(=O)Nc2ccc(C)c3c(C#N)c[nH]c23)cc1]

In some embodiments, LBM is

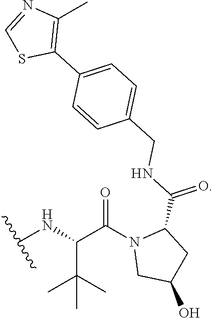 [SMILES: *N[C@H](C(=O)N1C[C@H](O)C[C@H]1C(=O)NCc1ccc(-c2scnc2C)cc1)C(C)(C)C]

In some embodiments, LBM is

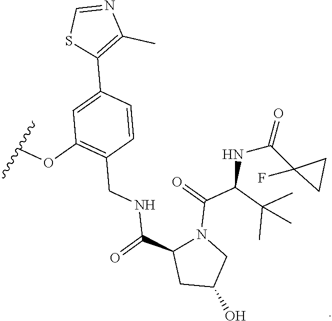 [SMILES: *Oc1cc(-c2scnc2C)ccc1CNC(=O)[C@@H]1C[C@@H](O)CN1C(=O)[C@@H](NC(=O)C1(F)CC1)C(C)(C)C]

In some embodiments, LBM is

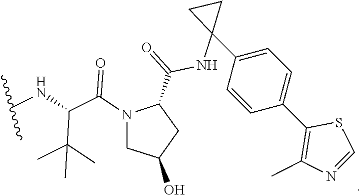 [SMILES: *N[C@H](C(=O)N1C[C@H](O)C[C@H]1C(=O)NC1(c2ccc(-c3scnc3C)cc2)CC1)C(C)(C)C]

In some embodiments, LBM is

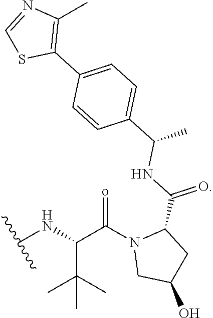

In some embodiments, LBM is

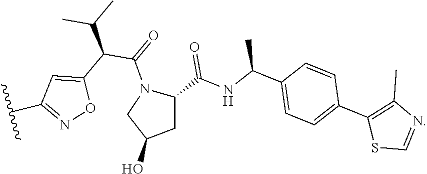 [SMILES: *c1cc([C@H](C(=O)N2C[C@H](O)C[C@H]2C(=O)N[C@@H](C)c2ccc(-c3scnc3C)cc2)C(C)C)on1]

In some embodiments, LBM is

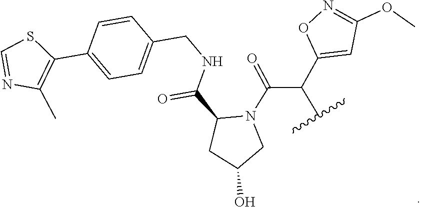 [SMILES: *C(C(=O)N1C[C@H](O)C[C@H]1C(=O)NCc1ccc(-c2scnc2C)cc1)c1cc(OC)no1]

In some embodiments, LBM is

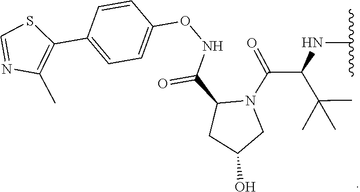 [SMILES: *N[C@H](C(=O)N1C[C@H](O)C[C@H]1C(=O)NOc1ccc(-c2scnc2C)cc1)C(C)(C)C]

In some embodiments, LBM is

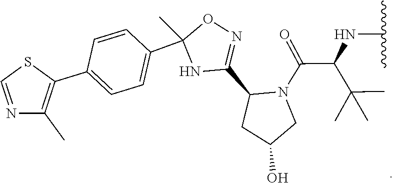 [SMILES: *N[C@H](C(=O)N1C[C@H](O)C[C@H]1C1=NOC(C)(c2ccc(-c3scnc3C)cc2)N1)C(C)(C)C]

In some embodiments, LBM is

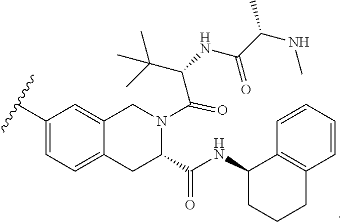 [SMILES: *c1ccc2c(c1)CN(C(=O)[C@@H](NC(=O)[C@H](C)NC)C(C)(C)C)[C@H](C(=O)N[C@@H]1CCCc3ccccc31)C2]

In some embodiments, LBM is

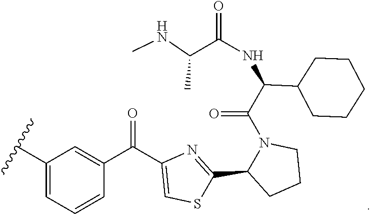 [SMILES: *c1cccc(C(=O)c2csc([C@@H]3CCCN3C(=O)[C@@H](NC(=O)[C@H](C)NC)C3CCCCC3)n2)c1]

In some embodiments, LBM is

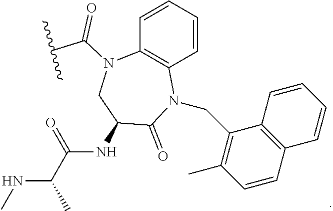 [SMILES: *C(=O)N1C[C@H](NC(=O)[C@H](C)NC)C(=O)N(Cc2c(C)ccc3ccccc23)c2ccccc21]

In some embodiments, LBM is

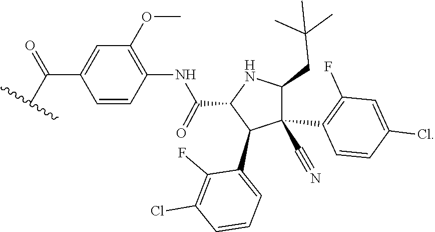 [SMILES: *C(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1]

In some embodiments, LBM is

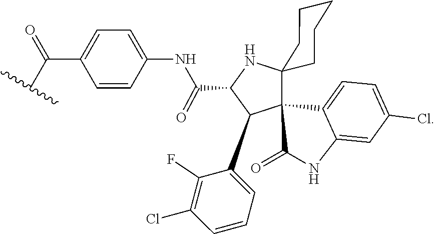 [SMILES: *C(=O)c1ccc(NC(=O)[C@@H]2NC3(CCCCC3)[C@@]3(C(=O)Nc4cc(Cl)ccc43)[C@H]2c2cccc(Cl)c2F)cc1]

In some embodiments, LBM is

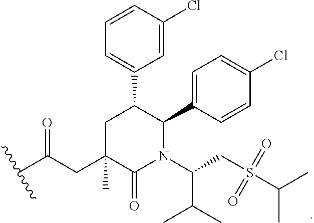 [SMILES: *C(=O)C[C@@]1(C)C[C@H](c2cccc(Cl)c2)[C@@H](c2ccc(Cl)cc2)N([C@H](CS(=O)(=O)C(C)C)C(C)C)C1=O]

In some embodiments, LBM is

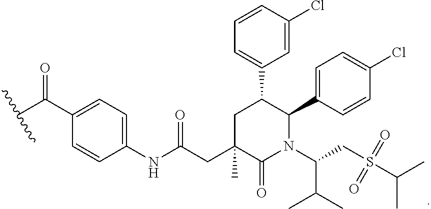 [SMILES: *C(=O)c1ccc(NC(=O)C[C@@]2(C)C[C@H](c3cccc(Cl)c3)[C@@H](c3ccc(Cl)cc3)N([C@H](CS(=O)(=O)C(C)C)C(C)C)C2=O)cc1]

In some embodiments, LBM is

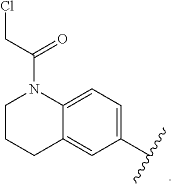 [SMILES: *c1ccc2c(c1)CCCN2C(=O)CCl]

In some embodiments, LBM is

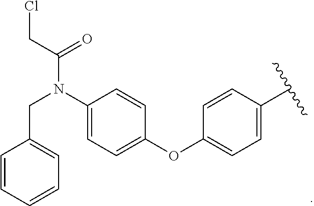 [SMILES: *c1ccc(Oc2ccc(N(Cc3ccccc3)C(=O)CCl)cc2)cc1]

In some embodiments, LBM is

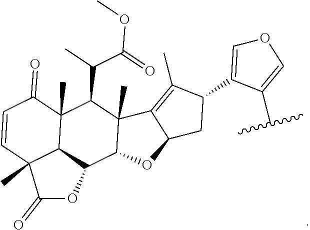 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45]

In some embodiments, LBM is

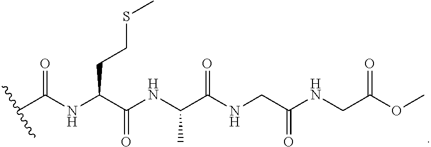 [SMILES: *C(=O)N[C@@H](CCSC)C(=O)N[C@@H](C)C(=O)NCC(=O)NCC(=O)OC]

In some embodiments, LBM is

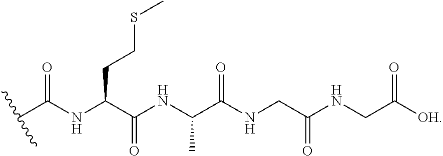 [SMILES: *C(=O)N[C@@H](CCSC)C(=O)N[C@@H](C)C(=O)NCC(=O)NCC(=O)O]

In some embodiments, LBM is

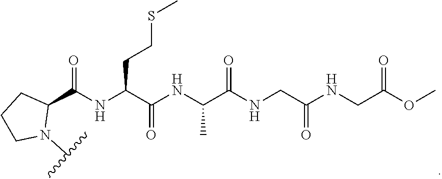 [SMILES: *N1CCC[C@H]1C(=O)N[C@@H](CCSC)C(=O)N[C@@H](C)C(=O)NCC(=O)NCC(=O)OC]

In some embodiments, LBM is

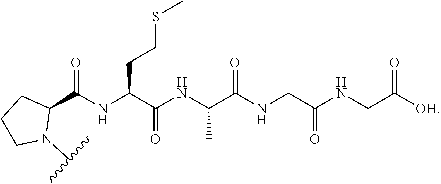 [SMILES: *N1CCC[C@H]1C(=O)N[C@@H](CCSC)C(=O)N[C@@H](C)C(=O)NCC(=O)NCC(=O)O]

In some embodiments, LBM is

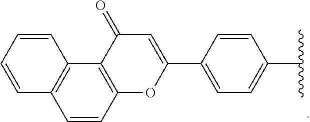 [SMILES: *c1ccc(-c2cc(=O)c3c(ccc4ccccc43)o2)cc1]

In some embodiments, LBM is

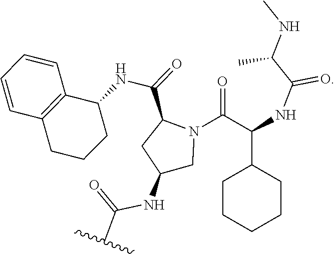 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1]

In some embodiments, LBM is

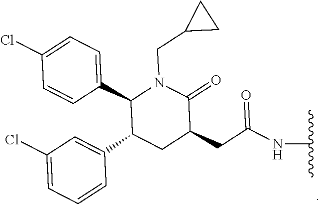 [SMILES: *NC(=O)C[C@H]1C[C@H](c2cccc(Cl)c2)[C@@H](c2ccc(Cl)cc2)N(CC2CC2)C1=O]

In some embodiments, LBM is

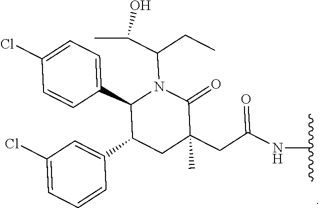 [SMILES: *NC(=O)C[C@@]1(C)C[C@H](c2cccc(Cl)c2)[C@@H](c2ccc(Cl)cc2)N(C(CC)[C@H](C)O)C1=O]

In some embodiments, LBM is

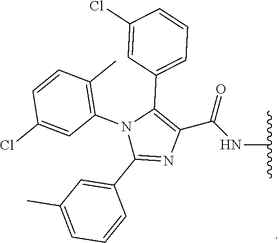 [SMILES: *NC(=O)c1nc(-c2cccc(C)c2)n(-c2cc(Cl)ccc2C)c1-c1cccc(Cl)c1]

In some embodiments, LBM is

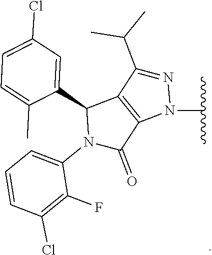 [SMILES: *n1nc(C(C)C)c2c1C(=O)N(c1cccc(Cl)c1F)[C@H]2c1cc(Cl)ccc1C]

In some embodiments, LBM is

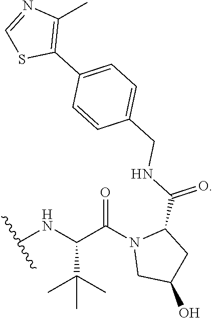 [SMILES: *N[C@H](C(=O)N1C[C@H](O)C[C@H]1C(=O)NCc1ccc(-c2scnc2C)cc1)C(C)(C)C]

In certain embodiments, the present invention provides a compound of formula I, wherein DIM (e.g., LBM) is a CRBN E3 ubiquitin ligase binding moiety thereby forming a compound of formula I-qqq:

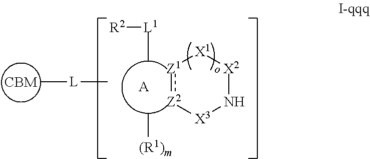

or a pharmaceutically acceptable salt thereof, wherein L and CBM are as defined above and described in embodiments herein, wherein:
each X¹ is independently —CH₂—, —O—, —NR—, —CF₂—,

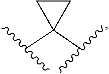 [SMILES: *C1(*)CC1]

### —C(O)—, —C(S)—, or

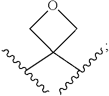 [SMILES: *C1(*)COC1] [0777] X² and X³ are independently —CH₂—, —C(O)—, —C(S)—, or

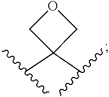 [SMILES: *C1(*)COC1]

Z¹ and Z² are independently a carbon atom or a nitrogen atom;
Ring A is a fused ring selected from benzo, a 4-6 membered saturated or partially unsaturated carbocyclic or heterocyclic ring having 1-3 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—;
each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CR₂F, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃; or
   two R¹ groups are optionally taken together to form an optionally substituted 5-8 membered partially unsaturated or aryl fused ring having 0-2 heteroatoms independently selected from nitrogen, oxygen, or sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R² is selected from

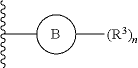

or hydrogen;
Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups;
each R³ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
[symbol] is a single or double bond;
m is 0, 1, 2, 3 or 4;
n is 0, 1, 2, 3 or 4; and
o is 0, 1, or 2.

A defined above and described herein each X¹ is independently a covalent bond, —CH₂—, —O—, —NR—, —CF₂—,

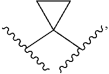 [SMILES: *C1(*)CC1]

### —C(O)—, —C(S)—, or

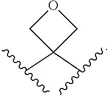 [SMILES: *C1(*)COC1]

In some embodiments, X¹ is a covalent bond. In some embodiments, X¹ is —CH₂—. In some embodiments, X¹ is —O—. In some embodiments, X¹ is —NR—. In some embodiments, X¹ is —CF₂—. In some embodiments, X¹ is

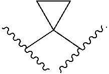 [SMILES: *C1(*)CC1]

In some embodiments, X¹ is —C(O)—. In some embodiments, X¹ is —C(S)—. In some embodiments, X¹ is

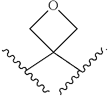 [SMILES: *C1(*)COC1]

In certain embodiments, X¹ is selected from those shown in the compounds of Table 1.

As defined above and described herein, X² and X³ are independently —CH₂—, —C(O)—, —C(S)—, or

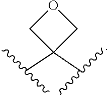 [SMILES: *C1(*)COC1]

In some embodiments, X² and X³ are independently —CH₂—. In some embodiments, X² and X³ are independently —C(O)—. In some embodiments, X² and X³ are independently —C(S)—. In some embodiments, X² and X³ are independently

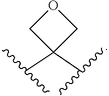 [SMILES: *C1(*)COC1]

In certain embodiments, X² and X³ are independently selected from those shown in the compounds of Table 1.

As defined above and described herein, X⁴ is a covalent bond, —CH₂—, —CR₂—, —O—, —NR—, —CF₂—,

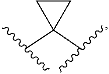 [SMILES: *C1(*)CC1]

### —C(O)—, —C(S)—, or

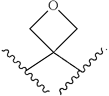 [SMILES: *C1(*)COC1]

As define above and described herein, Z¹ and Z² are independently a carbon atom or a nitrogen atom.

In some embodiments, Z¹ and Z² are independently a carbon atom. In some embodiments, Z¹ and Z² are independently a carbon atom.

In certain embodiments, Z¹ and Z² are independently selected from those shown in the compounds of Table 1.

As defined above and described herein, Ring A is fused ring selected from benzo or a 5-6 membered heteroaryl ring having 1-4 heteroatoms independently selected from nitrogen, oxygen, and sulfur.

In some embodiments, Ring A is benzo. In some embodiments, Ring A is a 5-6 membered heteroaryl ring having 1-4 heteroatoms independently selected from nitrogen, oxygen, and sulfur.

In certain embodiments, Ring A is selected from those shown in the compounds of Table 1.

As defined above and described herein, L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—

In some embodiments, L¹ is a covalent bond. In some embodiments, L¹ is a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is —C(O)—.

In certain embodiments, L¹ is selected from those shown in the compounds of Table 1.

As defined above and described herein, each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃, or two R¹ groups are optionally taken together to form an optionally substituted 5-8 membered partially unsaturated or aryl fused ring having 0-2 heteroatoms independently selected from nitrogen, oxygen, or sulfur.

In some embodiments, R¹ is hydrogen. In some embodiments, R¹ is deuterium. In some embodiments, R¹ is R⁴. In some embodiments, R¹ is halogen. In some embodiments, R¹ is —CN. In some embodiments, R¹ is —NO₂. In some embodiments, R¹ is —OR. In some embodiments, R¹ is —SR. In some embodiments, R¹ is —NR₂. In some embodiments, R¹ is —S(O)₂R. In some embodiments, R¹ is —S(O)₂NR₂. In some embodiments, R¹ is —S(O)R. In some embodiments, R¹ is —CF₂R. In some embodiments, R¹ is —CF₃. In some embodiments, R′ is —CR₂(OR). In some embodiments, R′ is —CR₂(NR₂). In some embodiments, R′ is —C(O)R. In some embodiments, R′ is —C(O)OR. In some embodiments, R′ is —C(O)NR₂. In some embodiments, R′ is —C(O)N(R)OR. In some embodiments, R′ is —OC(O)R. In some embodiments, R′ is —OC(O)NR₂. In some embodiments, R′ is —C(S)NR₂. In some embodiments, R′ is —N(R)C(O)OR. In some embodiments, R′ is —N(R)C(O)R. In some embodiments, R′ is —N(R)C(O)NR₂. In some embodiments, R′ is —N(R)S(O)₂R. In some embodiments, R′ is —OP(O)R₂. In some embodiments, R′ is —OP(O)(OR)₂. In some embodiments, R′ is —OP(O)(OR)NR₂. In some embodiments, R′ is —OP(O)(NR₂)₂. In some embodiments, R′ is —Si(OR)R₂. In some embodiments, R′ is —SiR₃. In some embodiments, two R¹ groups are optionally taken together to form an optionally substituted 5-8 membered partially unsaturated or aryl fused ring having 0-2 heteroatoms independently selected from nitrogen, oxygen, or sulfur.

In certain embodiments, each R¹ is independently selected from those shown in the compounds of Table 1.

As defined above and described here, each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted a 5-6 membered heteroaryl ring having 1-4 heteroatoms independently selected from nitrogen, oxygen, and sulfur. In some embodiments, two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

As defined above and described herein, R² is selected from

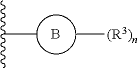

or hydrogen.

In some embodiment R² is

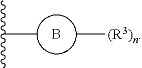

In some embodiments, R² is hydrogen.

In certain embodiments, R² is selected from those shown in the compounds of Table 1.

As defined above and described herein, Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups.

In some embodiments, Ring B is phenyl. In some embodiments, Ring B is a 4-10 membered saturated or partially unsaturated mono- or bicyclic carbocyclic or heterocyclic ring having 1-3 heteroatoms independently selected from nitrogen, oxygen, and sulfur. In some embodiments, Ring B is a 5-6 membered heteroaryl ring having 1-4 heteroatoms independently selected from nitrogen, oxygen, and sulfur. In some embodiments, Ring B is further optionally substituted with 1-2 oxo groups.

In certain embodiments, Ring B is selected from those shown in the compounds of Table 1.

As defined above and described herein, each R³ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃.

In some embodiments, R³ is hydrogen. In some embodiments, R³ is deuterium. In some embodiments, R³ is R⁴. In some embodiments, R³ is halogen. In some embodiments, R³ is —CN. In some embodiments, R³ is —NO₂. In some embodiments, R³ is —OR. In some embodiments, R³ is —SR. In some embodiments, R³ is —NR₂. In some embodiments, R³ is —S(O)₂R. In some embodiments, R³ is —S(O)₂NR₂. In some embodiments, R³ is —S(O)R. In some embodiments, R³ is —CF₂R. In some embodiments, R³ is —CF₃. In some embodiments, R³ is —CR₂(OR). In some embodiments, R³ is —CR₂(NR₂). In some embodiments, R³ is —C(O)R. In some embodiments, R³ is —C(O)OR. In some embodiments, R³ is —C(O)NR₂. In some embodiments, R³ is —C(O)N(R)OR. In some embodiments, R³ is —OC(O)R. In some embodiments, R³ is —OC(O)NR₂. In some embodiments, R³ is —N(R)C(O)OR. In some embodiments, R³ is —N(R)C(O)R. In some embodiments, R³ is —N(R)C(O)NR₂. In some embodiments, R³ is —N(R)S(O)₂R. In some embodiments, R³ is —OP(O)R₂. In some embodiments, R³ is —OP(O)(OR)₂. In some embodiments, R³ is —OP(O)(OR)NR₂. In some embodiments, R³ is —OP(O)(NR₂)₂. In some embodiments, R³ is —SiR₃.

In certain embodiments, R³ is selected from those shown in the compounds of Table 1.

As defined above and described herein, each R⁴ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁴ is an optionally substituted C₁₋₆ aliphatic. In some embodiments, R⁴ is an optionally substituted phenyl. In some embodiments, R⁴ is an optionally substituted 4-7 membered saturated or partially unsaturated heterocyclic ring having 1-2 heteroatoms independently selected from nitrogen, oxygen, and sulfur. In some embodiments, R⁴ is an optionally substituted 5-6 membered heteroaryl ring having 1-4 heteroatoms independently selected from nitrogen, oxygen, and sulfur.

In certain embodiments, R⁴ is selected from those shown in the compounds of Table 1.

As defined above and described herein, [symbol] is a single or double bond.

In some embodiments, [symbol] is a single bond. In some embodiments, [symbol] is a double bond.

In certain embodiments, [symbol] is selected from those shown in the compounds of Table 1.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In certain embodiments, m is selected from those shown in the compounds of Table 1.

As defined above and described herein, n is 0, 1, 2, 3 or 4.

In some embodiments, n is 0. In some embodiments, n is 1. In some embodiments, n is 2. In some embodiments, n is 3. In some embodiments, n is 4.

In certain embodiments, n is selected from those shown in the compounds of Table 1.

As defined above and described herein, o is 0, 1, or 2.

In some embodiments, n is 0. In some embodiments, n is 1. In some embodiments, m is 2.

In certain embodiments, o is selected from those shown in the compounds of Table 1.

In some embodiments, the present invention provides a compound of formula I-qqq, wherein Ring A is benzo, o is 1, X¹ is —CH₂—, X² and X³ are —C(O)—, and Z¹ and Z² are carbon atoms as shown, to provide a compound of formula I-qqq-1:

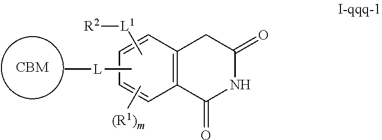

or a pharmaceutically acceptable salt thereof, wherein each of CBM, L, L, R¹, R², and m is as defined above and described in embodiments herein, both singly and in combination.

In some embodiments, the present invention provides a compound of formula I-qqq, wherein Ring A is benzo, o is 1, X¹, X² and X³ are —C(O)—, and Z¹ and Z² are carbon atoms as shown, to provide a compound of formula I-qqq-12:

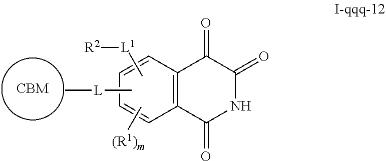

or a pharmaceutically acceptable salt thereof, wherein each of CBM, L, L¹, R¹, R², and m is as defined above and described in embodiments herein, both singly and in combination.

In some embodiments, LBM is

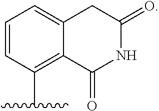 [SMILES: *c1cccc2c1C(=O)NC(=O)C2]

In some embodiments, LBM is

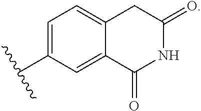 [SMILES: *c1ccc2c(c1)C(=O)NC(=O)C2]

In some embodiments, LBM is

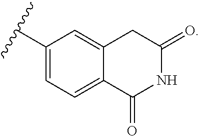 [SMILES: *c1ccc2c(c1)CC(=O)NC2=O]

In some embodiments, LBM is

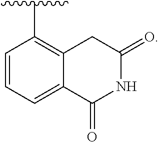 [SMILES: *c1cccc2c1CC(=O)NC2=O]

In some embodiments, LBM is

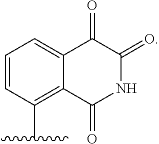 [SMILES: *c1cccc2c1C(=O)NC(=O)C2=O]

In some embodiments, LBM is

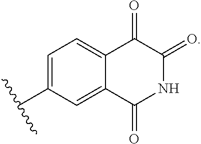 [SMILES: *c1ccc2c(c1)C(=O)NC(=O)C2=O]

In some embodiments, LBM is

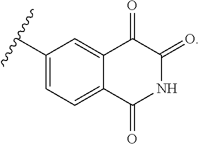 [SMILES: *c1ccc2c(c1)C(=O)C(=O)NC2=O]

In some embodiments, LBM is

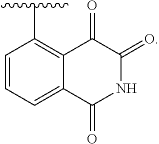 [SMILES: *c1cccc2c1C(=O)C(=O)NC2=O]

In some embodiments, LBM is selected from those in Table 1, below.

In certain embodiments, the present invention provides a compound of formula I, wherein DIM (e.g., LBM) is a RPN13 binding moiety thereby forming a compound of formula I-rrr:

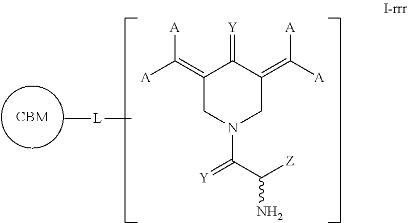

or a pharmaceutically acceptable salt thereof, wherein L and CBM are as defined above and described in embodiments herein, and wherein each of the variables A, Y, and Z is as described and defined in WO 2019/165229, the entirety of each of which is herein incorporated by reference.

In certain embodiments, the present invention provides a compound of formula I, wherein DIM (e.g., LBM) is a Ubr1 binding moiety as described in Shanmugasundaram, K. et al, J. Bio. Chem. 2019, doi: 10.1074/jbc.AC119.010790, the entirety of each of which is herein incorporated by reference, thereby forming a compound of formula I-sss-1 or I-sss-2:

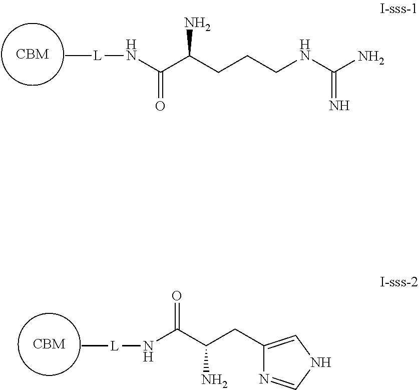

or a pharmaceutically acceptable salt thereof, wherein L and CBM are as defined above and described in embodiments herein.

In certain embodiments, the present invention provides a compound of formula I, wherein DIM (e.g., LBM) is a CRBN E3 ubiquitin ligase binding moiety thereby forming a compound of formula I-uuu-1, I-uuu-2, I-uuu-3 or I-uuu-4:

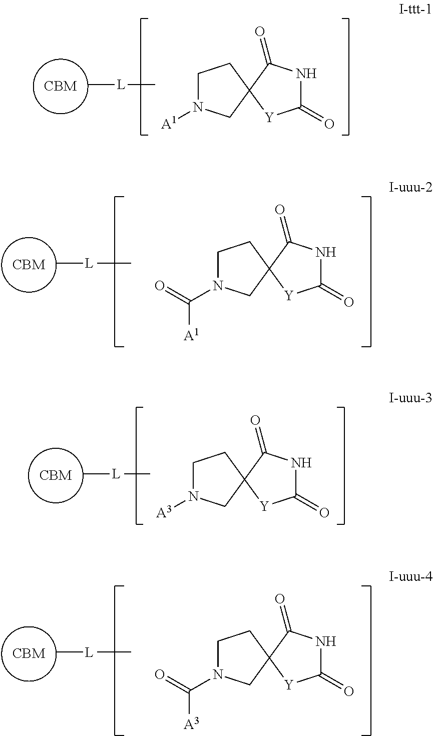

or a pharmaceutically acceptable salt thereof, wherein L and CBM are as defined above and described in embodiments herein, and wherein each of the variables Y, A¹, and A³ is as described and defined in WO 2019/236483, the entirety of each of which is herein incorporated by reference.

In certain embodiments, the present invention provides a compound of formula I, wherein DIM (e.g., LBM) is human kelch-like ECH-associated protein 1 (KEAP1) thereby forming a compound of formula I-vvv:

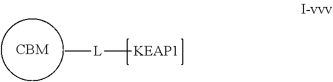

or a pharmaceutically acceptable salt thereof, wherein L and CBM are as defined above and described in embodiments herein, both singly and in combination.

In certain embodiments, the present invention provides a compound of formula I, wherein DIM (e.g., LBM) is KEAP1 binding moiety as recited in Lu et al., Euro. J. Med. Chem., 2018, 146:251-9, thereby forming a compound of formula I-www:

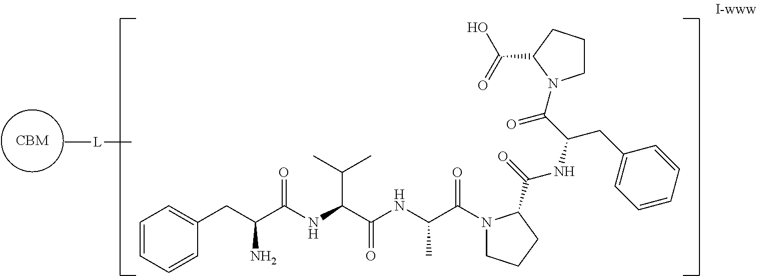

or a pharmaceutically acceptable salt thereof, wherein L and CBM are as defined above and described in embodiments herein, both singly and in combination.

In certain embodiments, the present invention provides a compound of formula I, wherein DIM (e.g., LBM) is KEAP1-NRF2 binding moiety thereby forming a compound of formula I-xxx or I-xxx-2:

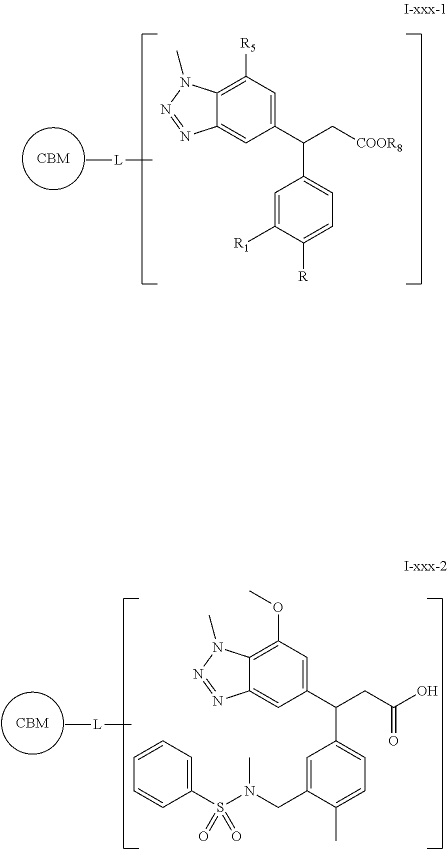

or a pharmaceutically acceptable salt thereof, wherein L and CBM are as defined above and described in embodiments herein, and wherein each of the variables R, R₁, R₅, and R₈ is as described and defined in WO 2020/018788, the entirety of each of which is herein incorporated by reference.

In certain embodiments, the present invention provides a compound of formula I, wherein DIM (e.g., LBM) is KEAP1-NRF2 binding moiety as recited in Tong et al., “Targeted Protein Degradation via a Covalent Reversible Degrader Based on Bardoxolone”, ChemRxiv 2020, thereby forming a compound of formula I-yyy-1 or I-yyy-2:

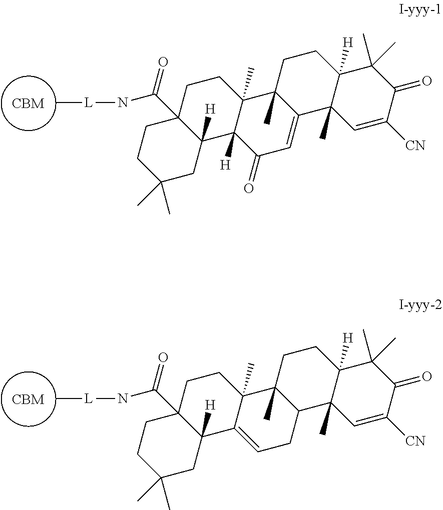

or a pharmaceutically acceptable salt thereof, wherein L and CBM are as defined above and described in embodiments herein, both singly and in combination.

In some embodiments, LBM is

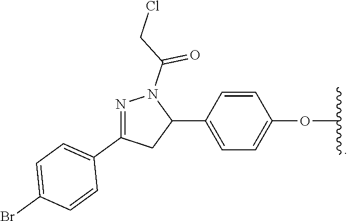 [SMILES: *Oc1ccc(C2CC(c3ccc(Br)cc3)=NN2C(=O)CCl)cc1]

In some embodiments, LBM is

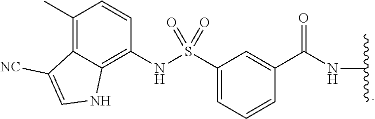 [SMILES: *NC(=O)c1cccc(S(=O)(=O)Nc2ccc(C)c3c(C#N)c[nH]c23)c1]

DCAF1 Binding Moiety

In some embodiments, DIM is DBM.

In some embodiments, DBM is a DCAF1 binding moiety.

In certain embodiments, the present invention provides a compound of formula I, wherein DBM is a DCAF1 binding moiety of formula I-aaaaa:

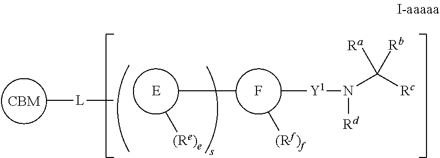

or a pharmaceutically acceptable salt thereof, wherein L and CBM are as defined and described herein, and wherein:
Ring E is phenyl, a 4-7 membered partially unsaturated carbocyclyl or heterocyclyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Ring F is phenylenyl, a 4-10 membered partially unsaturated carbocyclylenyl or heterocyclylenyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Y¹ is a C₁₋₃ hydrocarbon chain wherein each methylene is optionally substituted with —CR₂—, —CR(OR)—, —C(O)—, —C(NR)—, —C(NOR)—, —S(O)—, or —S(O)₂—;
R^{a} is an optionally substituted C₁₋₆ aliphatic or

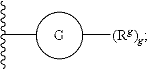

Ring G is phenyl, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur;
R^{b} is hydrogen, an optionally substituted C₁₋₆ aliphatic, phenyl, or a 5-6 membered heteroaryl with 1-4 heteroatoms independently selected from nitrogen, oxygen and sulfur, or:
R^{a} and R^{b} are optionally taken together with their intervening atoms to form an optionally substituted 9-10 membered saturated or partially unsaturated bicyclic ring having 0-3 heteroatoms independently selected from nitrogen, oxygen, and sulfur, or:
when Y¹ is —C(NR)—, R^{b} is optionally taken together with R of —C(NR)— with their intervening atoms to form a 5-7 membered partially unsaturated heterocyclyl with 0-1 heteroatoms, in addition to the 2 nitrogen atoms within the heterocyclyl, independently selected from nitrogen, oxygen, and sulfur;
R^{e} is —CR₂CONR₂, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur;
R^{d} is hydrogen, or:
when R^{c} is —CR₂CONR₂, R^{d} is optionally taken together with a single R of —CR₂CONR₂ with their intervening atoms to form a 5-7 membered saturated or partially unsaturated heterocyclyl with 0-3 heteroatoms, in addition to the nitrogen atom to which R^{a} is attached, independently selected from nitrogen, oxygen, and sulfur;
R^{e}, R^{f}, and R^{g} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-7 membered saturated or partially unsaturated ring having 0-3 heteroatoms, in addition to the atom to which they are attached, independently selected from nitrogen, oxygen, and sulfur;
s is 0 or 1; and
each of e, f, and g are independently 0, 1, 2, 3, or 4;
wherein DBM is further optionally substituted with

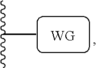

wherein

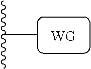

is a warhead group.

In certain embodiments, the present invention provides a compound of formula I, wherein DBM is a DCAF1 binding moiety of formula I-bbbbb:

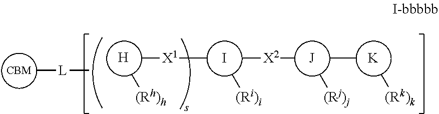

or a pharmaceutically acceptable salt thereof, wherein L and CBM are as defined and described herein, and wherein:
Ring H is a 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur;
Ring I is phenylenyl, a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur;
Ring J is a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur;
Ring K is phenyl, naphthyl, a 9-10 membered saturated or partially unsaturated bicyclic heterocyclylenyl with 1-3 heteroatoms independently selected from nitrogen, oxygen, and sulfur, or a 5-13 membered monocyclic, bicyclic, or tricyclic heteroarylenyl with 1-5 heteroatoms independently selected from nitrogen, oxygen and sulfur;
R^{h}, Rⁱ, R^{j}, and R^{k} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂, or:
   an Rⁱ group on Ring I and an RJ group or Ring J are optionally taken together with their intervening atoms to form a 5-8 membered saturated or partially unsaturated ring having 0-3 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same atom are optionally taken together with their intervening atoms to form an optionally substituted 3-7 membered saturated or partially unsaturated ring having 0-3 heteroatoms, in addition to the atom to which they are attached, independently selected from nitrogen, oxygen, and sulfur;
each of X¹ and X² is independently a covalent bond, spiro-fusion between the two rings that X¹ or X² connect, —CR₂—, —CR(OR)—, —CRF—, —CF₂—, —NR—, —O—, —S—, or —S(O)₂—;
s is 0 or 1; and
each of w, x, y, and z are independently 0, 1, 2, 3, or 4;
wherein DBM is further optionally substituted with

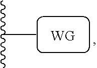

wherein

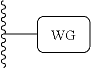

is a warhead group.

As described above and defined herein, Ring E is phenyl, a 4-7 membered partially unsaturated carbocyclyl or heterocyclyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur.

In some embodiments, Ring E is phenyl. In some embodiments, Ring E is a 4-7 membered partially unsaturated carbocyclyl. In some embodiments, Ring E is a 4-7 membered partially unsaturated heterocyclyl with 1-3 heteroatoms independently selected from nitrogen, oxygen, and sulfur. In some embodiments, Ring E is a 5-9 membered monocyclic or bicyclic heteroarylenyl with 1-4 heteroatoms independently selected from nitrogen, oxygen and sulfur.

In some embodiments, Ring E is cyclobutyl, azetinyl, cyclohexyl, cyclohexenyl, tetrahydro-2H-pyranyl, pyrrolidinyl, 4,5-dihydro-1H-pyrazolyl, piperidinyl, phenyl, isoxazolyl, isothiazolyl, pyrazolyl, pyridyl, pyridazinyl, pyrimidinyl, indolyl, benzoimidazolyl, pyrazolo[1,5-a]pyridyl, or [1,2,4]triazolo[1,5-a]pyridyl.

In some embodiments, Ring E is as depicted in the compounds of Table 3, below.

As described above and defined herein, Ring F is phenylenyl, a 4-10 membered partially unsaturated carbocyclylenyl or heterocyclylenyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur.

In some embodiments, Ring F is phenylenyl. In some embodiments, Ring F is a 4-10 membered partially unsaturated carbocyclylenyl. In some embodiments, Ring F is a 4-10 membered partially unsaturated heterocyclylenyl with 1-3 heteroatoms independently selected from nitrogen, oxygen, and sulfur. In some embodiments, Ring F is a 5-9 membered monocyclic or bicyclic heteroarylenyl with 1-4 heteroatoms independently selected from nitrogen, oxygen and sulfur.

In some embodiments, Ring F is cyclobutylenyl, azetinylenyl, cyclopentylenyl cyclohexyl, phenylenyl, pyrrolylenyl, imidazolylenyl, pyrazolylenyl, 1,2,3-triazolylenyl, 1,2,4-triazolylenyl, pyridylenyl, indazolyl, 1,2,3,6-tetrahydropyridinyl, 4,5,6,7-tetrahydro-1H-pyrazolo[4,3-b]pyridyl, benzoimidazolyl, 3,4-dihydroquinolinyl, or 4,5,6,7-tetrahydro-1H-pyrazolo[4,3-c]pyridyl.

In some embodiments, Ring F is as depicted in the compounds of Table 3, below.

As described above and defined herein, Ring G is phenyl, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur.

In some embodiments, Ring G is phenyl. In some embodiments, Ring G is a 5-7 membered saturated or partially unsaturated carbocyclyl. In some embodiments, Ring G is a 5-7 membered saturated or partially unsaturated heterocyclyl with 1-3 heteroatoms independently selected from nitrogen, oxygen, and sulfur. In some embodiments, Ring G is a 5-6 membered heteroaryl with 1-4 heteroatoms independently selected from nitrogen, oxygen and sulfur.

In some embodiments, Ring G is cyclohexyl, cyclohexenyl, isothiazolyl, phenyl, or pyridyl.

In some embodiments, Ring G is as depicted in the compounds of Table 3, below.

As described above and defined herein, Ring H is a 3-11 membered saturated or partially unsaturated monocyclic, bicyclic, bridged bicyclic, or spirocyclic carbocyclyl or heterocyclyl with 1-3 heteroatoms independently selected from nitrogen, oxygen, and sulfur.

In some embodiments, Ring H is a 3-11 membered saturated or partially unsaturated monocyclic, bicyclic, bridged bicyclic, or spirocyclic carbocyclyl. In some embodiments, Ring H is a 3-11 membered saturated or partially unsaturated monocyclic, bicyclic, bridged bicyclic, or spirocyclic heterocyclyl with 1-3 heteroatoms independently selected from nitrogen, oxygen, and sulfur.

In some embodiments, Ring H is cyclopropyl, cyclobutyl, azetinyl, pyrrolidinyl, cyclohexyl, piperidinyl, piperazinyl, 3,6-dihydro-2H-pyranyl, tetrahydro-2H-pyranyl, morpholinyl, piperzinyl, 2,7-diazaspiro[3.5]nonanyl, 3,4-dihydro-2H-pyrido[3,2-b][1,4]oxazinyl, 2-oxa-5-azabicyclo[2.2.1]heptanyl, 6-oxa-3-azabicyclo[3.1.1]heptanyl, or 2-oxa-5-azabicyclo[2.2.2]octanyl.

In some embodiments, Ring H is as depicted in the compounds of Table 3, below.

As described above and defined herein, Ring I is phenylenyl, a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur, or a 5-9 membered monocyclic or bicyclic heteroarylenyl with 1-4 heteroatoms independently selected from nitrogen, oxygen and sulfur.

In some embodiments, Ring I is phenylenyl. In some embodiments, Ring I is a 3-11 membered saturated or partially unsaturated monocyclic, bicyclic, bridged bicyclic, or spirocyclic carbocyclylenyl. In some embodiments, Ring I is a 3-11 membered saturated or partially unsaturated monocyclic, bicyclic, bridged bicyclic, or spirocyclic heterocyclylenyl with 1-3 heteroatoms independently selected from nitrogen, oxygen, and sulfur. In some embodiments, Ring I is a 5-9 membered monocyclic or bicyclic heteroarylenyl with 1-4 heteroatoms independently selected from nitrogen, oxygen and sulfur.

In some embodiments, Ring I is phenylenyl, imidazolylenyl, pyrazolylenyl, oxazolylenyl, thiazolylenyl, 1,2-thiazinanylenyl, pyridylenyl, pyridazinylenyl, pyrimidinylenyl, 2,6-diazaspiro[3.5]nonanylenyl, 2,3-dihydro-1H-pyrrolo[2,3-b]pyridylenyl, 2,3-dihydro-1H-pyrrolo[3,2-c]pyridylenyl, 1H-pyrrolo[2,3-b]pyridylenyl, 3H-imidazo[4,5-b]pyridylenyl, 9H-purinylenyl, 1,2,3,4-tetrahydro-1,8-naphthyridinylenyl, or 1,2,3,4-tetrahydro-1,6-naphthyridinylenyl.

In some embodiments, Ring I is as depicted in the compounds of Table 3, below.

As described above and defined herein, Ring J is a 3-11 membered saturated or partially unsaturated monocyclic, bicyclic, bridged bicyclic, or spirocyclic carbocyclylenyl or heterocyclylenyl with 1-3 heteroatoms independently selected from nitrogen, oxygen, and sulfur.

In some embodiments, Ring J is a 3-11 membered saturated or partially unsaturated monocyclic, bicyclic, bridged bicyclic, or spirocyclic carbocyclyl. In some embodiments, Ring J is a 3-11 membered saturated or partially unsaturated monocyclic, bicyclic, bridged bicyclic, or spirocyclic heterocyclyl with 1-3 heteroatoms independently selected from nitrogen, oxygen, and sulfur.

In some embodiments, Ring J is cyclohexylenyl, azetidinylenyl, pyrrolidinylenyl, imidazolylenyl, piperidinylenyl, piperzinylenyl, azepanylenyl, 8-azabicyclo[3.2.1]octanylenyl, 2-azabicyclo[3.2.1]octanylenyl, 2-azabicyclo[3.2.2]nonanylenyl, octahydro-1H-pyrrolo[3,2-b]pyridylenyl, decahydro-1,5-naphthyridinylenyl, 9-azabicyclo[3.3.1]nonanylenyl, 5-azaspiro[3.5]nonanylenyl, 2-oxa-5-azaspiro[3.5]nonanylenyl, or 2,6-diazaspiro[3.5]nonanylenyl.

In some embodiments, Ring J is as depicted in the compounds of Table 3, below.

As described above and defined herein, Ring K is phenyl, naphthyl, a 9-10 membered saturated or partially unsaturated bicyclic heterocyclylenyl with 1-3 heteroatoms independently selected from nitrogen, oxygen, and sulfur, or a 5-13 membered monocyclic, bicyclic, or tricyclic heteroarylenyl with 1-5 heteroatoms independently selected from nitrogen, oxygen and sulfur.

In some embodiments, Ring K is phenyl. In some embodiments, Ring K is naphthyl. In some embodiments, Ring K is a 9-10 membered saturated or partially unsaturated bicyclic heterocyclylenyl with 1-3 heteroatoms independently selected from nitrogen, oxygen, and sulfur. In some embodiments, Ring K is a 5-13 membered monocyclic, bicyclic, or tricyclic heteroarylenyl with 1-5 heteroatoms independently selected from nitrogen, oxygen and sulfur.

In some embodiments, Ring K is 1,2,3-triazolyl, thiazolyl, pyrazolyl, phenyl, pyridyl, pyridazinyl, pyrimidinyl, indazolyl, benzo[d]isoxazolyl, benzo[d]isothiazolyl, pyrazolo[1,5-a]pyrimidinyl, 2,3-dihydro-1H-pyrrolo[2,3-c]pyridinyl, 6,7-dihydro-5H-cyclopenta[b]pyridinyl, 2,3-dihydro-1H-pyrrolo[3,2-c]pyridinyl, naphthyl, quinolinyl, isoquinolinyl, 1,6-naphthyridinyl, phthalazinyl, quinazolinyl, 2,7-naphthyridinyl, or tetrazolo[1,5-a]quinoxalinyl.

In some embodiments, Ring K is as depicted in the compounds of Table 3, below.

As described above and defined herein, R^{a} is an optionally substituted C₁₋₆ aliphatic or

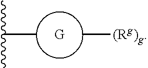

In some embodiments, R^{a} is an optionally substituted C₁₋₆ aliphatic. In some embodiments, R^{a} is

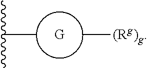

In some embodiments, Ring R^{a} is methyl.

In some embodiments, Ring R^{a} is as depicted in the compounds of Table 3, below.

As described above and defined herein, R^{b} is hydrogen, an optionally substituted C₁₋₆ aliphatic, phenyl, or a 5-6 membered heteroaryl with 1-4 heteroatoms independently selected from nitrogen, oxygen and sulfur, or R^{a} and R^{b} are optionally taken together with their intervening atoms to form an optionally substituted 9-10 membered saturated or partially unsaturated bicyclic ring having 0-3 heteroatoms independently selected from nitrogen, oxygen, and sulfur, or when Y is —C(NR)—, R^{b} is optionally taken together with R of —C(NR)— with their intervening atoms to form a 5-7 membered partially unsaturated heterocyclyl with 0-1 heteroatoms, in addition to the 2 nitrogen atoms within the heterocyclyl, independently selected from nitrogen, oxygen, and sulfur.

In some embodiments, R^{b} is hydrogen. In some embodiments, R^{b} is hydrogen is an optionally substituted C₁₋₆ aliphatic. In some embodiments, R^{b} is hydrogen is phenyl. In some embodiments, R^{b} is hydrogen is a 5-6 membered heteroaryl with 1-4 heteroatoms independently selected from nitrogen, oxygen and sulfur. In some embodiments, R^{a} and R^{b} are optionally taken together with their intervening atoms to form an optionally substituted 9-10 membered saturated or partially unsaturated bicyclic ring having 0-3 heteroatoms independently selected from nitrogen, oxygen, and sulfur. In some embodiments, when Y is —C(NR)—, R^{b} is optionally taken together with R of —C(NR)— with their intervening atoms to form a 5-7 membered partially unsaturated heterocyclyl with 0-1 heteroatoms, in addition to the 2 nitrogen atoms within the heterocyclyl, independently selected from nitrogen, oxygen, and sulfur.

In some embodiment, R^{b} is methyl, cyclopropyl, phenyl, —CO₂H, —CH₂cyclopropyl, —CH₂OH, —CH₂OMe, or —CH₂CO₂H.

In some embodiments, Ring R^{b} is as depicted in the compounds of Table 3, below.

As described above and defined herein, R^{∘} is —CR₂CONR₂, a 5-7 membered saturated or partially unsaturated carbocyclyl or heterocyclyl with 1-3 heteroatoms independently selected from nitrogen, oxygen, and sulfur, or a 5-6 membered heteroaryl with 1-4 heteroatoms independently selected from nitrogen, oxygen and sulfur.

In some embodiments, R is —CR₂CONR₂. In some embodiments, R is a 5-7 membered saturated or partially unsaturated carbocyclyl. In some embodiments, R is a 5-7 membered saturated or partially unsaturated heterocyclyl with 1-3 heteroatoms independently selected from nitrogen, oxygen, and sulfur. In some embodiments, R is a 5-6 membered heteroaryl with 1-4 heteroatoms independently selected from nitrogen, oxygen and sulfur.

In some embodiments, R is —CH₂CONH₂, —CH(Me)CONH₂, —CH₂CONHMe, —CH₂CONHEt, —CH₂CONHCH₂Ph, —CH₂CONHcyclopropyl, pyrrolidin-2-onyl, piperidin-2-only, or isoxazolyl.

In some embodiments, Ring R is as depicted in the compounds of Table 3, below.

As described above and defined herein, R^{a} is hydrogen, or when R^{∘} is —CR₂CONR₂, R^{a} is optionally taken together with a single R of —CR₂CONR₂ with their intervening atoms to form a 5-7 membered saturated or partially unsaturated heterocyclyl with 0-3 heteroatoms, in addition to the nitrogen atom to which R^{d} is attached, independently selected from nitrogen, oxygen, and sulfur.

In some embodiments, R^{d} is hydrogen.

In some embodiments, Ring R^{d} is as depicted in the compounds of Table 3, below.

As described above and defined herein, R^{e}, R^{f}, R^{g}, R^{h}, Rⁱ, R^{j}, and R^{k} are each independently selected from hydrogen, oxo, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, —NRP(O)(NR₂)₂, —P(O)R₂, —P(O)(OR)₂, —P(O)(OR)NR₂, and —P(O)(NR₂)₂, or an Rⁱ group on Ring I and an R^{j} group or Ring J are optionally taken together with their intervening atoms to form a 5-8 membered saturated or partially unsaturated ring having 0-3 heteroatoms independently selected from nitrogen, oxygen, and sulfur.

In some embodiments, one or more of R^{e}, R^{r}, R^{g}, R^{h}, Rⁱ, R^{j}, and R^{k} is hydrogen. In some embodiments, one or more of R^{e}, R^{r}, R^{g}, R^{h}, Rⁱ, R^{j}, and R^{k} is oxo. In some embodiments, one or more of R^{e}, R^{r}, R^{g}, R^{h}, Rⁱ, R^{j}, and R^{k} is R^{A}. In some embodiments, one or more of R^{e}, R^{r}, R^{g}, R^{h}, Rⁱ, R^{j}, and R^{k} is halogen. In some embodiments, one or more of R^{e}, R^{r}, R^{g}, R^{h}, Rⁱ, R^{j}, and R^{k} is —CN. In some embodiments, one or more of R^{e}, R^{f}, R^{g}, R^{h}, Rⁱ, R^{j}, and R^{k} is —NO₂. In some embodiments, one or more of R^{e}, R^{r}, R^{g}, R^{h}, Rⁱ, R^{j}, and R^{k} is —OR. In some embodiments, one or more of R^{e}, R^{r}, R^{g}, R^{h}, Rⁱ, R^{j}, and R^{k} is —SR. In some embodiments, one or more of R^{e}, R^{r}, R^{g}, R^{h}, Rⁱ, R^{j}, and R^{k} is —NR₂. In some embodiments, one or more of R^{e}, R^{r}, R^{g}, R^{h}, Rⁱ, R^{j}, and R^{k} is —SiR₃. In some embodiments, one or more of R^{e}, R^{r}, R^{g}, R^{h}, Rⁱ, R^{j}, and R^{k} is —S(O)₂R. In some embodiments, one or more of R^{e}, R^{r}, R^{g}, R^{h}, Rⁱ, R^{j}, and R^{k} is —S(O)₂NR₂. In some embodiments, one or more of R^{e}, R^{r}, R^{g}, R^{h}, Rⁱ, R^{j}, and R^{k} is —S(O)R. In some embodiments, one or more of R^{e}, R^{r}, R^{g}, R^{h}, Rⁱ, R^{j}, and R^{k} is —C(O)R. In some embodiments, one or more of R^{e}, R^{r}, R^{g}, R^{h}, Rⁱ, R^{j}, and R^{k} is —C(O)OR. In some embodiments, one or more of R^{e}, R^{r}, R^{g}, R^{h}, Rⁱ, R^{j}, and R^{k} is —C(O)NR₂. In some embodiments, one or more of R^{e}, R^{r}, R^{g}, R^{h}, Rⁱ, R^{j}, and R^{k} is —C(O)NROR. In some embodiments, one or more of R^{e}, R^{r}, R^{g}, R^{h}, Rⁱ, R^{j}, and R^{k} is —OC(O)R. In some embodiments, one or more of R^{e}, R^{r}, R^{g}, R^{h}, Rⁱ, R^{j}, and R^{k} is —OC(O)NR₂. In some embodiments, one or more of R^{e}, R^{r}, R^{g}, R^{h}, Rⁱ, R^{j}, and R^{k} is —OP(O)R₂. In some embodiments, one or more of R^{e}, R^{r}, R^{g}, R^{h}, Rⁱ, R^{j}, and R^{k} is —OP(O)(OR)₂. In some embodiments, one or more of R^{e}, R^{r}, R^{g}, R^{h}, Rⁱ, R^{j}, and R^{k} is —OP(O)(OR)NR₂. In some embodiments, one or more of R^{e}, R^{r}, R^{g}, R^{h}, Rⁱ, R^{j}, and R^{k} is —OP(O)(NR₂)₂. In some embodiments, one or more of R^{e}, R^{r}, R^{g}, R^{h}, Rⁱ, R^{j}, and R^{k} is —NRC(O)OR. In some embodiments, one or more of R^{e}, R^{r}, R^{g}, R^{h}, Rⁱ, R^{j}, and R^{k} is —NRC(O)R. In some embodiments, one or more of R^{e}, R^{r}, R^{g}, R^{h}, Rⁱ, R^{j}, and R^{k} is —NRC(O)N(R)₂. In some embodiments, one or more of R^{e}, R^{r}, R^{g}, R^{h}, Rⁱ, R^{j}, and R^{k} is —NRS(O)₂R. In some embodiments, one or more of R^{e}, R^{r}, R^{g}, R^{h}, Rⁱ, R^{j}, and R^{k} is —NP(O)R₂. In some embodiments, one or more of R^{e}, R^{r}, R^{g}, R^{h}, R^{h}, Rⁱ, R^{j}, and R^{k} is —NRP(O)(OR)₂. In some embodiments, one or more of R^{e}, R^{r}, R^{g}, R^{h}, Rⁱ, R^{j}, and R^{k} is —NRP(O)(OR)NR₂. In some embodiments, one or more of R^{e}, R^{r}, R^{g}, R^{h}, Rⁱ, R^{j}, and R^{k} is —NRP(O)(NR₂)₂. In some embodiments, one or more of R^{e}, R^{r}, R^{g}, R^{h}, Rⁱ, R^{j}, and R^{k} is —P(O)R₂. In some embodiments, one or more of R^{e}, R^{r}, R^{g}, R^{h}, Rⁱ, R^{j}, and R^{k} is —P(O)(OR)₂. In some embodiments, one or more of R^{e}, R^{r}, R^{g}, R^{h}, Rⁱ, R^{j}, and R^{k} is —P(O)(OR)NR₂. In some embodiments, one or more of R^{e}, R^{r}, R^{g}, R^{h}, Rⁱ, R^{j}, and R^{k} is —P(O)(NR₂)₂. In some embodiments, an Rⁱ group on Ring I and an R^{j} group or Ring J are taken together with their intervening atoms to form a 5-8 membered saturated or partially unsaturated ring having 0-3 heteroatoms independently selected from nitrogen, oxygen, and sulfur.

In some embodiments, R^{∘} is hydrogen, oxo, fluoro, chloro, —CN, methyl, —CO₂H, —CO₂Me, —CONH₂, —C(O)CHCH₂, —OH, —OMe, —CH₂CHF₂, —CH₂OMe, —CH₂CO₂H, —CH₂SO₂Me, —CH₂CH₂O₂H, —CH₂CH₂SO₂Me, —CH₂CH₂OMe, —NHC(O)CHCH₂, tetrazolyl, or N-methyltetrazolyl.

In some embodiments, R is hydrogen, oxo, methyl, isopropyl, —CH₂cyclopropyl, —CH₂cyclopentyl, —CH₂cyclohexyl, —CH₂morpholinyl, —CH₂Ph, —CH₂thiazolyl, —CH₂pyrimidinyl, —CH₂CH₂OMe, —CH₂CH₂Ph, —C(O)Me, —C(O)CHCH₂, —C(O)Ph, —C(O)pyrimidinyl, —NH₂, —NHC(O)CHCH₂, —CH₂NHC(O)CHCH₂, —CCNHC(O)CHCH₂, —NHcyclohexyl, —NHphenyl, or -NHpyrimidinyl,

In some embodiments, R^{h} is hydrogen, oxo, fluoro, methyl, ethyl, n-propyl, b-butyl, —CH₂CH₂OMe, —C(O)CHCH₂, —NHC(O)CHCH₂, —N(Me)C(O)CHCH₂, —CH₂NHC(O)CHCH₂, or

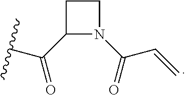 [SMILES: *C(=O)C1CCN1C(=O)C=C]

In some embodiments, R⁹ is hydrogen, oxo, fluoro, chloro, —CN, methyl, —CONH₂, —OH, or —OMe.

In some embodiments, R is hydrogen, oxo, fluoro, chloro, methyl, —CF₃, —CH₂OH, —CN, —OH, —OMe, —NH₂, or —N(Me)CH₂CH₂CH₂N(Me)C(O)CHCH₂.

In some embodiments, RJ is hydrogen, oxo, fluoro, methyl, —CH₂F, —CH₂OH, —CO₂H, —C(O)NH₂, —OH, —OMe, or —S(O)₂NH₂.

In some embodiments, Rⁱ and R^{j}, are taken together by —CH₂CH₂— or —CH₂CH₂CH₂—.

In some embodiments, R^{k} is hydrogen, oxo, fluoro, chloro, —CN, methyl, isobutyl, —CF₃, —CH₂CF₃, —CH₂OH, —CH₂CO₂Me, —CH(OH)Me, —CH(NH₂)cyclopropyl, —CH₂Ph, —OH, —OMe, —OCF₃, —OiPr, OPh, —NHC(O)Me, —NHC(O)CHCH₂, —S(O)₂NH₂, 1,2,3-triazolyl, piperdinyl, N-methylpiperdinyl, phenyl, or pyridyl.

In some embodiments, R^{e}, R^{r}, R^{g}, R^{h}, Rⁱ, R^{j}, and R^{k} are as depicted in the compounds of Table 3, below.

As described above and defined herein, each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{A} is an optionally substituted C₁₋₆ aliphatic. In some embodiments, R^{A} is an optionally substituted phenyl. In some embodiments, R^{A} is an optionally substituted 3-7 membered saturated or partially unsaturated carbocyclic. In some embodiments, R^{A} is an optionally substituted saturated or partially unsaturated heterocyclic ring having 1-2 heteroatoms independently selected from nitrogen, oxygen, and sulfur. In some embodiments, R^{A} is an optionally substituted 5-6 membered heteroaryl ring having 1-4 heteroatoms independently selected from nitrogen, oxygen, and sulfur.

In some embodiments, R^{A} is C₁₋₆ alkyl (e.g., methyl, ethyl, isopropyl). In some embodiments, R^{A} is C₁₋₆ haloalkyl (e.g., —CF₃, —CHF₂).

In some embodiment, R^{A} is as depicted in the compounds of Table 3, below.

As described above and defined herein, each R is independently hydrogen, or an optionally substituted group selected from C₁₋₆ aliphatic, phenyl, a 4-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are optionally taken together with their intervening atoms to form an optionally substituted 3-7 membered saturated or partially unsaturated ring having 0-3 heteroatoms, in addition to the atom to which they are attached, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated carbocyclic. In some embodiments, R is an optionally substituted 4-7 membered saturated or partially unsaturated heterocyclic ring having 1-2 heteroatoms independently selected from nitrogen, oxygen, and sulfur. In some embodiments, R is an optionally substituted 5-6 membered heteroaryl ring having 1-4 heteroatoms independently selected from nitrogen, oxygen, and sulfur. In some embodiments, two R groups on the same atom are optionally taken together with their intervening atoms to form optionally substituted 3-7 membered saturated or partially unsaturated ring having 0-3 heteroatoms, in addition to the atom to which they are attached, independently selected from nitrogen, oxygen, and sulfur.

In some embodiment, R is as depicted in the compounds of Table 3, below.

As described above and defined herein, each of X and X² is independently a is a covalent bond, spiro-fusion between the two rings that X¹ or X² connect, —CR₂—, —CR(OR)—, —CRF—, —CF₂—, —NR—, —O—, —S—, or —S(O)₂—.

In some embodiments, X¹ and/or X² is a covalent bond. In some embodiments, X¹ and/or X² is —CR₂—. In some embodiments, X¹ and/or X² is —CR(OR)—. In some embodiments, X¹ and/or X² is —CRF—. In some embodiments, X¹ and/or X² is —CF₂—. In some embodiments, X¹ and/or X² is —NR—. In some embodiments, X¹ and/or X² is —O—. In some embodiments, X¹ and/or X² is —S—. In some embodiments, X¹ and/or X² is —S(O)₂—. In some embodiments, X and/or X² represents spiro-fusion between the two rings that X¹ or X² connect.

In some embodiments, X¹ is a covalent bond, —NH—, or —NMe-.

In some embodiments, X² is a covalent bond, —CH₂—, —CMe(OMe)-, —CMe(F)—, —CMe(CF₃)—, cyclopropylenyl, difluorocyclopropylenyl, —NH—, —NMe-, —N(COMe)-, —N(CF₃)—, -NEt-, —N(nPr)-, —N(nBu)-, —N(Ph)-, —N(3-pyridyl)-, —N(4-pyridyl)-, —N(SO₂Me)-, —N(CH₂CHF₂)—, —N(CH₂cyclopropyl)-, —N(CH₂Ph)-, —N(CH₂CONH₂)—, —N(CH₂SO₂Me)-, —N(CH₂CH₂CHF₂)—, —N(CH₂CH₂Ph)-, —N(CH₂CH₂CO₂H)—, —N(CH₂CH₂CONH₂)—, —N(CH₂CH₂CN)—, —N(CH₂CH₂OMe)-, —N(CH₂CH₂SO₂Me)-, —O—, —S—, or —S(O)₂—.

In some embodiments, X² represents spiro-fusion between the two rings that X² connects, e.g.,

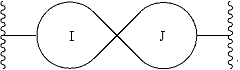

In some embodiment, X¹ and X² are as depicted in the compounds of Table 3, below.

As described above and defined herein, Y¹ is a C₁₋₃ hydrocarbon chain wherein each methylene is optionally substituted with —CR₂—, —CR(OR)—, —C(O)—, —C(NR)—, —C(NOR)—, —S(O)—, or —S(O)₂—.

In some embodiments, Y¹ is a C₁₋₃ hydrocarbon chain wherein each methylene is optionally substituted with —CR₂—, —CR(OR)—, —C(O)—, —C(NR)—, —C(NOR)—, —S(O)—, or —S(O)₂—.

In some embodiments, Y¹ is a C₁₋₃ hydrocarbon chain. In some embodiments, Y¹ is —CR₂—. In some embodiments, Y¹ is —CR(OR)—. In some embodiments, Y¹ is —C(O)—. In some embodiments, Y¹ is —C(NR)—. In some embodiments, Y¹ is —C(NOR)—. In some embodiments, Y¹ is —S(O)—. In some embodiments, Y¹ is —S(O)₂—.

In some embodiments, Y¹ is —CH₂—, —CH₂C(O)—, —NHCH₂C(O)—, —CH₂CH₂C(O)—, —CH₂CH(OH)C(O)—, —C(O)—, —C(NH)—, —C(NOH)—, —S(O)—, or —S(O)₂—.

In some embodiment, Y¹ is as depicted in the compounds of Table 3, below.

As described above and defined herein, s is 0 or 1.

In some embodiments, s is 0. In some embodiments, s is 1.

In some embodiment, s is as depicted in the compounds of Table 3, below.

As described above and defined herein, each of e, f, g, h, i, j, and k are independently 0, 1, 2, 3, or 4.

In some embodiments, e is 0. In some embodiments, e is 1. In some embodiments, e is 2. In some embodiments, e is 3. In some embodiments, e is 4.

In some embodiments, f is 0. In some embodiments, f is 1. In some embodiments, f is 2. In some embodiments, f is 3. In some embodiments, f is 4.

In some embodiments, g is 0. In some embodiments, g is 1. In some embodiments, g is 2. In some embodiments, g is 3. In some embodiments, g is 4.

In some embodiments, h is 0. In some embodiments, h is 1. In some embodiments, h is 2. In some embodiments, h is 3. In some embodiments, h is 4.

In some embodiments, i is 0. In some embodiments, i is 1. In some embodiments, i is 2. In some embodiments, i is 3. In some embodiments, i is 4.

In some embodiments, j is 0. In some embodiments, j is 1. In some embodiments, j is 2. In some embodiments, j is 3. In some embodiments, j is 4.

In some embodiments, k is 0. In some embodiments, k is 1. In some embodiments, k is 2. In some embodiments, k is 3. In some embodiments, k is 4.

In some embodiment, e, f, g, h, i, j, and k are as depicted in the compounds of Table 3, below.

In some embodiments, DBM is

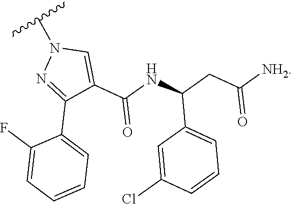 [SMILES: *n1cc(C(=O)N[C@@H](CC(N)=O)c2cccc(Cl)c2)c(-c2ccccc2F)n1]

In some embodiments, DBM is

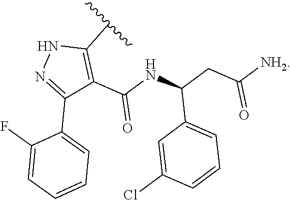 [SMILES: *c1[nH]nc(-c2ccccc2F)c1C(=O)N[C@@H](CC(N)=O)c1cccc(Cl)c1]

In some embodiments, DBM is

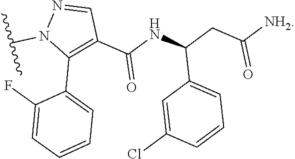 [SMILES: *n1ncc(C(=O)N[C@@H](CC(N)=O)c2cccc(Cl)c2)c1-c1ccccc1F]

In some embodiments, DBM is

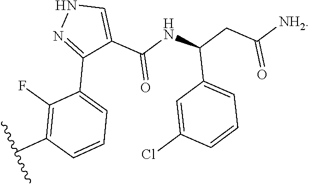 [SMILES: *c1cccc(-c2n[nH]cc2C(=O)N[C@@H](CC(N)=O)c2cccc(Cl)c2)c1F]

In some embodiments, DBM is

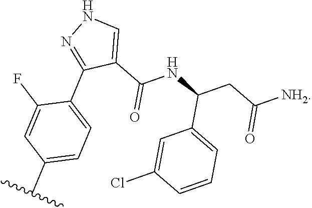 [SMILES: *c1ccc(-c2n[nH]cc2C(=O)N[C@@H](CC(N)=O)c2cccc(Cl)c2)c(F)c1]

In some embodiments, DBM is

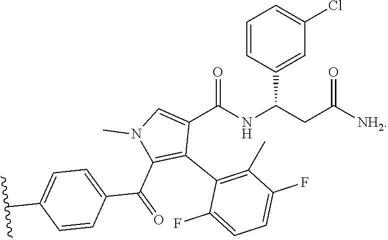 [SMILES: *c1ccc(C(=O)c2c(-c3c(F)ccc(F)c3C)c(C(=O)N[C@@H](CC(N)=O)c3cccc(Cl)c3)cn2C)cc1]

In some embodiments, DBM is

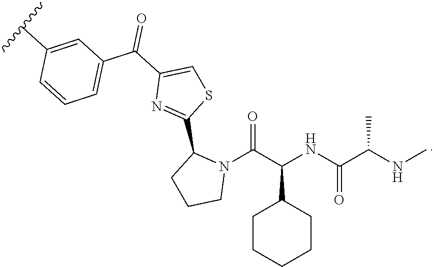 [SMILES: *c1cccc(C(=O)c2csc([C@@H]3CCCN3C(=O)[C@@H](NC(=O)[C@H](C)NC)C3CCCCC3)n2)c1]

In some embodiments, DBM is

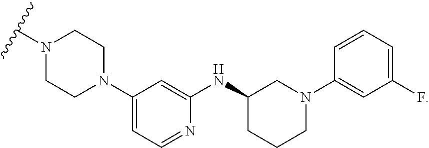 [SMILES: *N1CCN(c2ccnc(N[C@@H]3CCCN(c4cccc(F)c4)C3)c2)CC1]

In some embodiments, DBM is

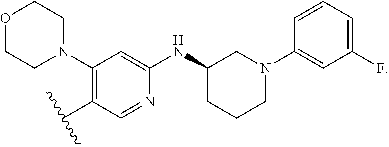 [SMILES: *c1cnc(N[C@@H]2CCCN(c3cccc(F)c3)C2)cc1N1CCOCC1]

In some embodiments, DBM is

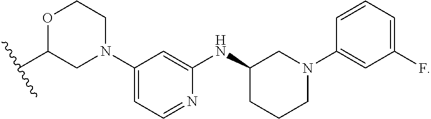 [SMILES: *C1CN(c2ccnc(N[C@@H]3CCCN(c4cccc(F)c4)C3)c2)CCO1]

In some embodiments, DBM is

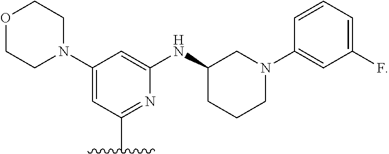 [SMILES: *c1cc(N2CCOCC2)cc(N[C@@H]2CCCN(c3cccc(F)c3)C2)n1]

In some embodiments, DBM is

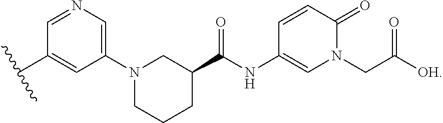 [SMILES: *c1cncc(N2CCC[C@H](C(=O)Nc3ccc(=O)n(CC(=O)O)c3)C2)c1]

In certain embodiments, the present invention provides a compound of formula I-aaaaa represented by any one of the following formulae:

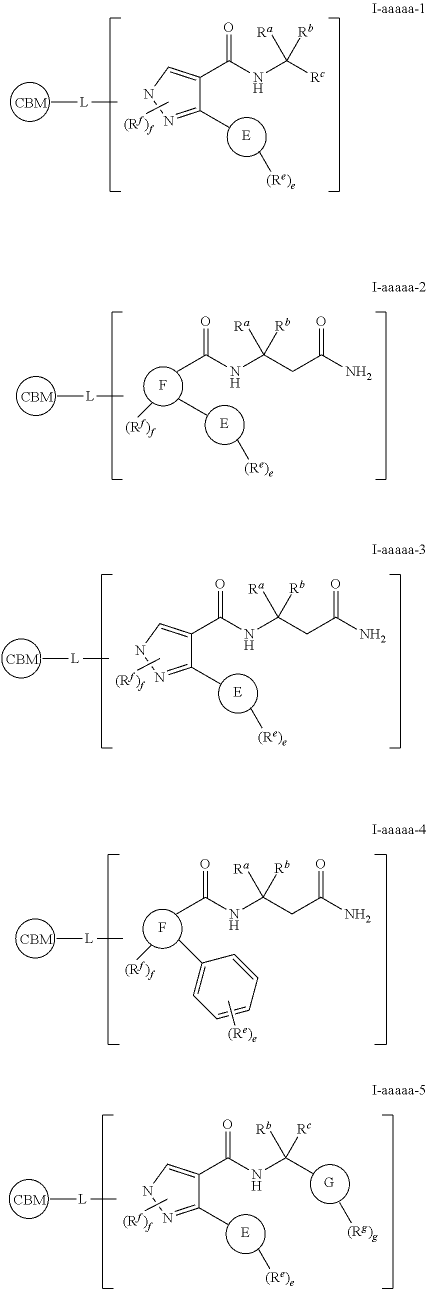

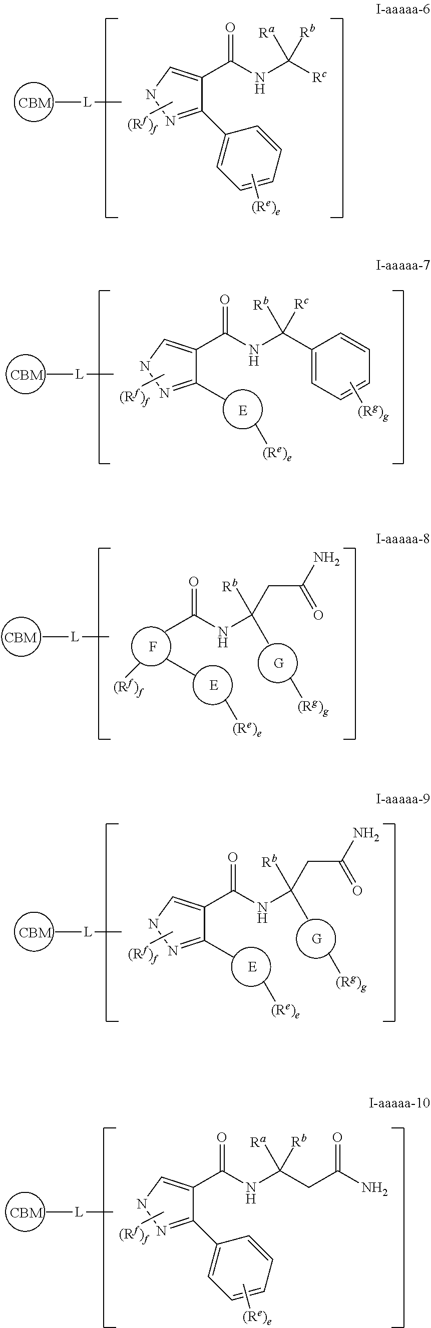

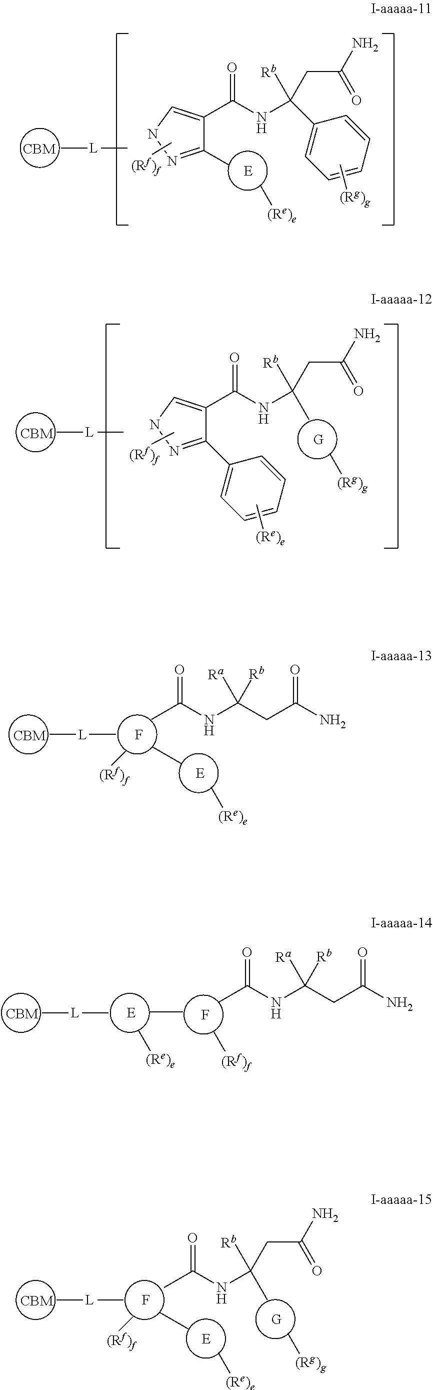

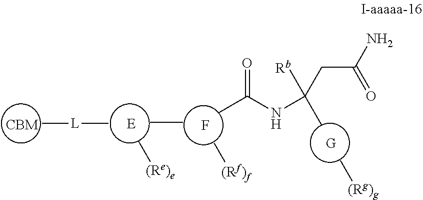

or a pharmaceutically acceptable salt thereof.

In certain embodiments, the present invention provides a compound of formula I-bbbbb represented by any one of the following formulae:

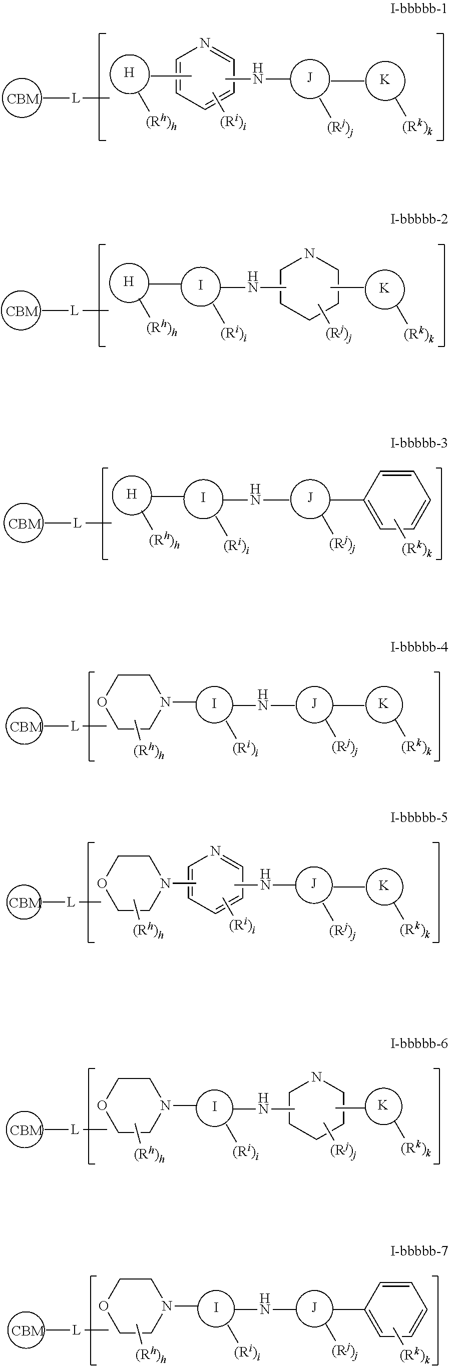

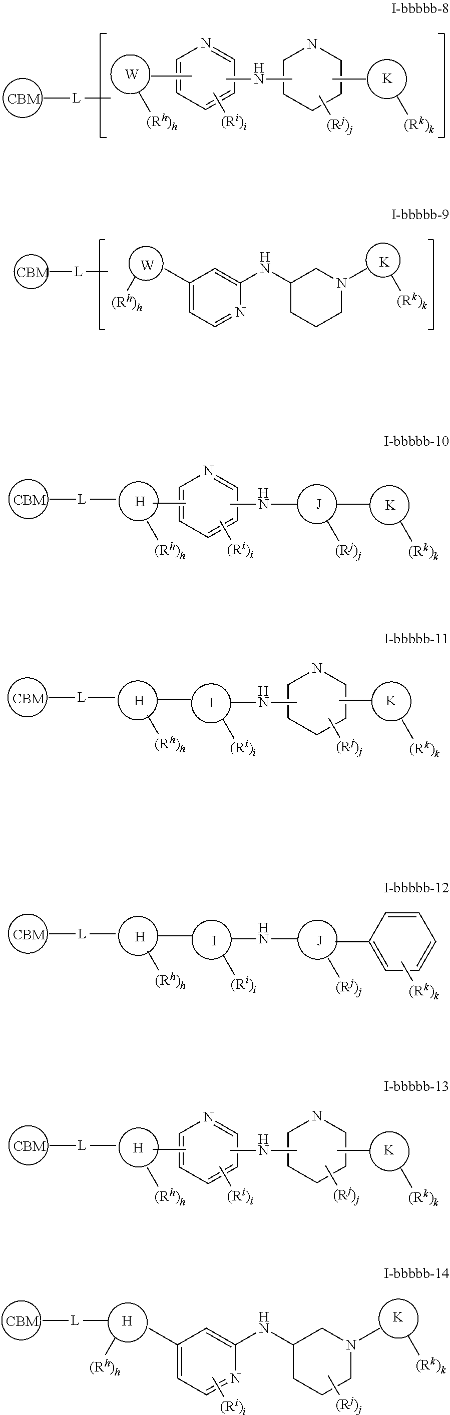

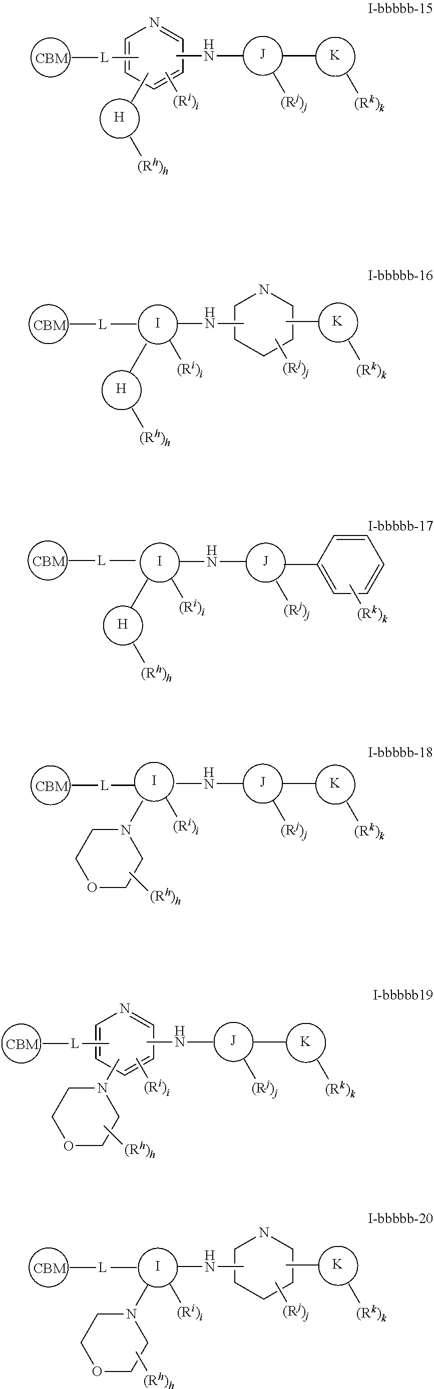

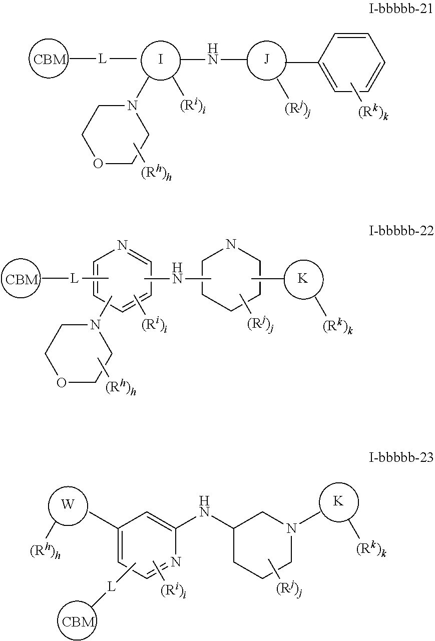

or a pharmaceutically acceptable salt thereof.

As defined above and described herein, DBM is further optionally substituted with

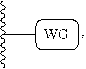

wherein

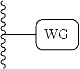

is a warhead group, attached to a modifiable carbon, oxygen, nitrogen or sulfur atom in formula I-aaaaa or I-bbbbb or substitution or replacement of any defined group in formula I-aaaaa or I-bbbbb (e.g., substitution or replacement of R^{e}, R^{r}, R^{g}, R^{h}, Rⁱ, R^{j}, or R^{k}).

In some embodiments, the warhead group is -L²-Y, wherein:
L² is a covalent bond or a bivalent C₁₋₈ saturated or unsaturated, straight or branched, hydrocarbon chain, wherein one, two, or three methylene units of L² are optionally and independently replaced by cyclopropylene, —NR—, —N(R)C(O)—, —C(O)N(R)—, —N(R)SO₂—, —SO₂N(R)—, —O—, —C(O)—, —OC(O)—, —C(O)O—, —S—, —SO—, —SO₂—, —C(═S)—, —C(═NR)—, —N═N—, or —C(═N₂)—;
Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with 1-4 R^{e} groups; and
each R^{e} is independently selected from -Q-Z, oxo, NO₂, halogen, CN, a suitable leaving group, or a C₁₋₆ aliphatic optionally substituted with oxo, halogen, NO₂, or CN, wherein: Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and Z is hydrogen or C₁₋₆ aliphatic optionally substituted with oxo, halogen, NO₂, or CN.

In certain embodiments, L² is a covalent bond.

In certain embodiments, L² is a bivalent C₁₋ₛ saturated or unsaturated, straight or branched, hydrocarbon chain. In certain embodiments, L² is —CH₂—.

In certain embodiments, L² is a covalent bond, —CH₂—, —NH—, —CH₂NH—, —NHCH₂—, —NHC(O)—, —NHC(O)CH₂OC(O)—, —CH₂NHC(O)—, —NHSO₂—, —NHSO₂CH₂—, —NHC(O)CH₂OC(O)—, or —SO₂NH—.

In some embodiments, L² is a bivalent C₂₋ₛ straight or branched, hydrocarbon chain wherein L² has at least one double bond and one or two additional methylene units of L² are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—.

In certain embodiments, L² is a bivalent C₂₋₈ straight or branched, hydrocarbon chain wherein L² has at least one double bond and at least one methylene unit of L² is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L² are optionally and independently replaced by cyclopropylene, —O—, —N(R)—, or —C(O)—.

In some embodiments, L² is a bivalent C₂₋₈ straight or branched, hydrocarbon chain wherein L² has at least one double bond and at least one methylene unit of L² is replaced by —C(O)—, and one or two additional methylene units of L² are optionally and independently replaced by cyclopropylene, —O—, —N(R)—, or —C(O)—.

As described above, in certain embodiments, L² is a bivalent C₂₋₈ straight or branched, hydrocarbon chain wherein L² has at least one double bond. One of ordinary skill in the art will recognize that such a double bond may exist within the hydrocarbon chain backbone or may be “exo” to the backbone chain and thus forming an alkylidene group. By way of example, such an L² group having an alkylidene branched chain includes —CH₂C(═CH₂)CH₂—. Thus, in some embodiments, L² is a bivalent C₂₋₈ straight or branched, hydrocarbon chain wherein L² has at least one alkylidenyl double bond. Exemplary L² groups include —NHC(O)C(═CH₂)CH₂—.

In certain embodiments, L² is a bivalent C₂₋₈ straight or branched, hydrocarbon chain wherein L² has at least one double bond and at least one methylene unit of L² is replaced by —C(O)—. In certain embodiments, L² is —C(O)CH═CH(CH₃)—, —C(O)CH═CHCH₂NH(CH₃)—, —C(O)CH═CH(CH₃)—, —C(O)CH═CH—, —CH₂C(O)CH═CH—, —CH₂C(O)CH═CH(CH₃)—, —CH₂CH₂C(O)CH═CH—, —CH₂CH₂C(O)CH═CHCH₂—, —CH₂CH₂C(O)CH═CHCH₂NH(CH₃)—, or —CH₂CH₂C(O)CH═CH(CH₃)—, or —CH(CH₃)OC(O)CH═CH—.

In certain embodiments, L² is a bivalent C₂₋₈ straight or branched, hydrocarbon chain wherein L² has at least one double bond and at least one methylene unit of L² is replaced by —OC(O)—.

In some embodiments, L² is a bivalent C₂₋₈ straight or branched, hydrocarbon chain wherein L² has at least one double bond and at least one methylene unit of L² is replaced by —NRC(O)—, —C(O)NR—, —N(R)SO₂—, —SO₂N(R)—, —S—, —S(O)—, —SO₂—, —OC(O)—, or —C(O)O—, and one or two additional methylene units of L² are optionally and independently replaced by cyclopropylene, —O—, —N(R)—, or —C(O)—. In some embodiments, L² is —CH₂OC(O)CH═CHCH₂—, —CH₂—OC(O)CH═CH—, or —CH(CH═CH₂)OC(O)CH═CH—.

In certain embodiments, L² is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)C(O)—, —NRC(O)CH═CHCH₂N(CH₃)—, —NRSO₂CH═CH—, —NRSO₂CH═CHCH₂—, —NRC(O)CH═CHCH₂O—, —NRC(O)C(═CH₂)CH₂—, —CH₂NRC(O)—, —CH₂NRC(O)CH═CH—, —CH₂CH₂NRC(O)—, or —CH₂NRC(O)cyclopropylene-, wherein each R is independently hydrogen or optionally substituted C₁₋₆ aliphatic.

In certain embodiments, L² is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)C(O)—, —NHC(O)CH═CHCH₂N(CH₃)—, —NHSO₂CH═CH—, —NHSO₂CH═CHCH₂—, —NHC(O)CH═CHCH₂O—, —NHC(O)C(═CH₂)CH₂—, —CH₂NHC(O)—, —CH₂NHC(O)CH═CH—, —CH₂CH₂NHC(O)—, or —CH₂NHC(O)cyclopropylene-.

In some embodiments, L² is a bivalent C₂₋₈ straight or branched, hydrocarbon chain wherein L² has at least one triple bond. In certain embodiments, L² is a bivalent C₂₋₈ straight or branched, hydrocarbon chain wherein L² has at least one triple bond and one or two additional methylene units of L² are optionally and independently replaced by —NRC(O)—, —C(O)NR—, —S—, —S(O)—, —SO₂—, —C(═S)—, —C(═NR)—, —O—, —N(R)—, or —C(O)—. In some embodiments, L² has at least one triple bond and at least one methylene unit of L² is replaced by —N(R)—, —N(R)C(O)—, —C(O)—, —C(O)O—, or —OC(O)—, or —O—.

Exemplary L² groups include —C≡C—, —C≡CCH₂N(isopropyl)-, —NHC(O)C≡CCH₂CH₂—, —CH₂—C≡C≡CH₂—, —C≡CCH₂O—, —CH₂C(O)C≡C—, —C(O)C≡C—, or —CH₂OC(═O)C≡C—.

In certain embodiments, L² is a bivalent C₂₋₈ straight or branched, hydrocarbon chain wherein one methylene unit of L² is replaced by cyclopropylene and one or two additional methylene units of L² are independently replaced by —C(O)—, —NRC(O)—, —C(O)NR—, —N(R)SO₂—, or —SO₂N(R)—. Exemplary L² groups include —NHC(O)-cyclopropylene-SO₂— and —NHC(O)-cyclopropylene-.

As defined generally above, Y is hydrogen, C₁₋₆ aliphatic optionally substituted with oxo, halogen, NO₂, or CN, or a 3-10 membered monocyclic or bicyclic, saturated, partially unsaturated, or aryl ring having 0-3 heteroatoms independently selected from nitrogen, oxygen, or sulfur, and wherein said ring is substituted with at 1-4 R^{e} groups, each R^{e} is independently selected from -Q-Z, oxo, NO₂, halogen, CN, a suitable leaving group, or C₁₋₆ aliphatic, wherein Q is a covalent bond or a bivalent C₁-6 saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and, Z is hydrogen or C₁₋₆ aliphatic optionally substituted with oxo, halogen, NO₂, or CN.

In certain embodiments, Y is hydrogen.

In certain embodiments, Y is C₁₋₆ aliphatic optionally substituted with oxo, halogen, NO₂, or CN. In some embodiments, Y is C₂₋₆alkenyl optionally substituted with oxo, halogen, NO₂, or CN. In other embodiments, Y is C₂₋₆alkynyl optionally substituted with oxo, halogen, NO₂, or CN. In some embodiments, Y is C₂₋₆alkenyl. In other embodiments, Y is C₂₋₄ alkynyl.

In other embodiments, Y is C₁₋₆ alkyl substituted with oxo, halogen, NO₂, or CN. Such Y groups include —CH₂F, —CH₂Cl, —CH₂CN, and —CH₂NO₂.

In certain embodiments, Y is a saturated 3-6 membered monocyclic ring having 0-3 heteroatoms independently selected from nitrogen, oxygen, or sulfur, wherein Y is substituted with 1-4 R^{e} groups, wherein each R^{e} is as defined above and described herein.

In some embodiments, Y is a saturated 3-4 membered heterocyclic ring having 1 heteroatom selected from oxygen or nitrogen wherein said ring is substituted with 1-2 R^{e} groups, wherein each R^{e} is as defined above and described herein. Exemplary such rings are epoxide and oxetane rings, wherein each ring is substituted with 1-2 R^{e} groups, wherein each R^{e} is as defined above and described herein.

In other embodiments, Y is a saturated 5-6 membered heterocyclic ring having 1-2 heteroatom selected from oxygen or nitrogen wherein said ring is substituted with 1-4 R^{e} groups, wherein each R^{e} is as defined above and described herein. Such rings include piperidine and pyrrolidine, wherein each ring is substituted with 1-4 R^{e} groups, wherein each R^{e} is as defined above and described herein. In certain embodiments, Y is

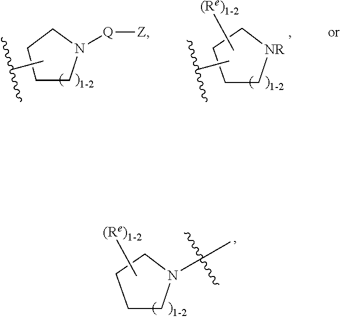

wherein each R, Q, Z, and R^{e} is as defined above and described herein.

In some embodiments, Y is a saturated 3-6 membered carbocyclic ring, wherein said ring is substituted with 1-4 R^{e} groups, wherein each R^{e} is as defined above and described herein. In certain embodiments, Y is cyclopropyl, cyclobutyl, cyclopentyl, or cyclohexyl, wherein each ring is substituted with 1-4 R^{e} groups, wherein each R^{e} is as defined above and described herein. In certain embodiments, Y is

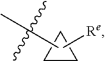

wherein R^{e} is as defined above and described herein.

In certain embodiments, Y is cyclopropyl optionally substituted with halogen, CN or NO₂.

In certain embodiments, Y is a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein.

In some embodiments, Y is a partially unsaturated 3-6 membered carbocyclic ring, wherein said ring is substituted with 1-4 R^{e} groups, wherein each R^{e} is as defined above and described herein. In some embodiments, Y is cyclopropenyl, cyclobutenyl, cyclopentenyl, or cyclohexenyl wherein each ring is substituted with 1-4 R^{e} groups, wherein each R^{e} is as defined 0-3 above and described herein. In certain

embodiments, Y is

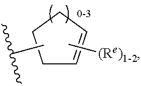

wherein each R^{e} is as defined above and described herein.

In certain embodiments, Y is a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein. In certain embodiments, Y is selected from:

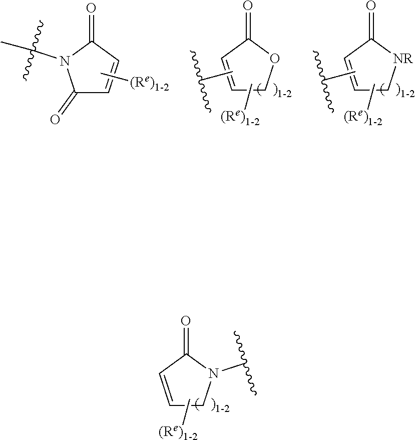

wherein each R and R^{e} is as defined above and described herein.

In certain embodiments, Y is a 6-membered aromatic ring having 0-2 nitrogens wherein said ring is substituted with 1-4 R^{e} groups, wherein each R^{e} group is as defined above and described herein. In certain embodiments, Y is phenyl, pyridyl, or pyrimidinyl, wherein each ring is substituted with 1-4 R^{e} groups, wherein each R^{e} is as defined above and described herein.

In some embodiments, Y is selected from:

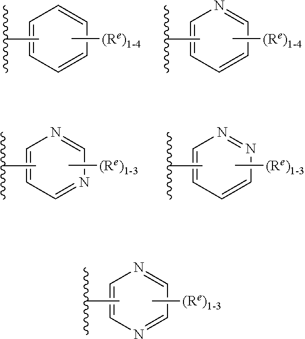

wherein each R^{e} is as defined above and described herein.

In other embodiments, Y is a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein. In some embodiments, Y is a 5 membered partially unsaturated or aryl ring having 1-3 heteroatoms independently selected from nitrogen, oxygen, and sulfur, wherein said ring is substituted with 1-4 R^{e} groups, wherein each R^{e} group is as defined above and described herein. Exemplary such rings are isoxazolyl, oxazolyl, thiazolyl, imidazolyl, pyrazolyl, pyrrolyl, furanyl, thienyl, triazole, thiadiazole, and oxadiazole, wherein each ring is substituted with 1-3 Regroups, wherein each R^{e} group is as defined above and described herein. In certain embodiments, Y is selected from:

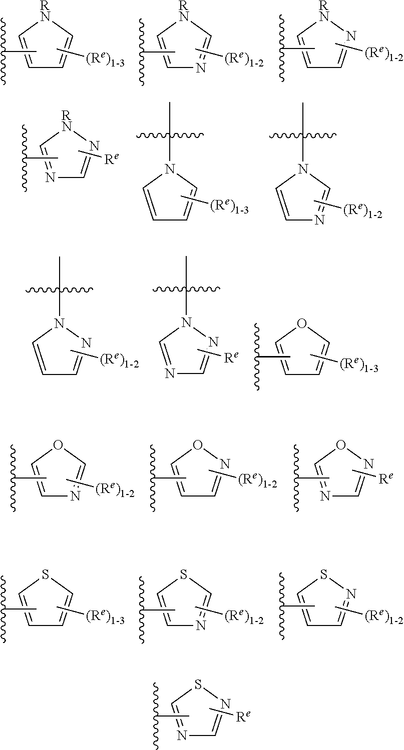

wherein each R and R^{e} is as defined above and described herein.

In certain embodiments, Y is an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein. According to another aspect, Y is a 9-10 membered bicyclic, partially unsaturated, or aryl ring having 1-3 heteroatoms independently selected from nitrogen, oxygen, or sulfur, wherein said ring is substituted with 1-4 R^{e} groups, wherein R^{e} is as defined above and described herein. Exemplary such bicyclic rings include 2,3-dihydrobenzo[d]isothiazole, wherein said ring is substituted with 1-4 R^{e} groups, wherein R^{e} is as defined above and described herein.

As defined generally above, each R^{e} group is independently selected from -Q-Z, oxo, NO₂, halogen, CN, a suitable leaving group, or C₁₋₆ aliphatic optionally substituted with oxo, halogen, NO₂, or CN, wherein Q is a covalent bond or a bivalent C₁₋₆ saturated or unsaturated, straight or branched, hydrocarbon chain, wherein one or two methylene units of Q are optionally and independently replaced by —N(R)—, —S—, —O—, —C(O)—, —OC(O)—, —C(O)O—, —SO—, or —SO₂—, —N(R)C(O)—, —C(O)N(R)—, —N(R)SO₂—, or —SO₂N(R)—; and Z is hydrogen or C₁₋₆ aliphatic optionally substituted with oxo, halogen, NO₂, or CN.

In certain embodiments, R^{e} is C₁₋₆ aliphatic optionally substituted with oxo, halogen, NO₂, or CN. In other embodiments, R^{e} is oxo, NO₂, halogen, or CN.

In some embodiments, R^{e} is -Q-Z, wherein Q is a covalent bond and Z is hydrogen (i.e., Reis hydrogen). In other embodiments, R^{e} is -Q-Z, wherein Q is a bivalent C₁-6 saturated or unsaturated, straight or branched, hydrocarbon chain, wherein one or two methylene units of Q are optionally and independently replaced by —NR—, —NRC(O)—, —C(O)NR—, —S—, —O—, —C(O)—, —SO—, or —SO₂—. In other embodiments, Q is a bivalent C₂₋₆ straight or branched, hydrocarbon chain having at least one double bond, wherein one or two methylene units of Q are optionally and independently replaced by —NR—, —NRC(O)—, —C(O)NR—, —S—, —O—, —C(O)—, —SO—, or —SO₂—. In certain embodiments, the Z moiety of the R^{e} group is hydrogen. In some embodiments, -Q-Z is —NHC(O)CH═CH₂ or —C(O)CH═CH₂.

In certain embodiments, each R^{e} is independently selected from oxo, NO₂, CN, fluoro, chloro, —NHC(O)CH═CH₂, —C(O)CH═CH₂, —CH₂CH═CH₂, —C≡CH, —C(O)OCH₂Cl, —C(O)OCH₂F, —C(O)OCH₂CN, —C(O)CH₂Cl, —C(O)CH₂F, —C(O)CH₂CN, or —CH₂C(O)CH₃.

In certain embodiments, R^{e} is a suitable leaving group, i.e., a group that is subject to nucleophilic displacement. A “suitable leaving” is a chemical group that is readily displaced by a desired incoming chemical moiety such as the thiol moiety of a cysteine of interest. Suitable leaving groups are well known in the art, e.g., see, “Advanced Organic Chemistry,” Jerry March, 5^{th} Ed., pp. 351-357, John Wiley and Sons, N.Y. Such leaving groups include, but are not limited to, halogen, alkoxy, sulphonyloxy, optionally substituted alkylsulphonyloxy, optionally substituted alkenylsulfonyloxy, optionally substituted arylsulfonyloxy, acyl, and diazonium moieties. Examples of suitable leaving groups include chloro, iodo, bromo, fluoro, acetoxy, methanesulfonyloxy (mesyloxy), tosyloxy, triflyloxy, nitro-phenylsulfonyloxy (nosyloxy), and bromo-phenylsulfonyloxy (brosyloxy).

In certain embodiments, the following embodiments and combinations of -L²-Y apply:
(a) L² is a bivalent C₂₋ₛ straight or branched, hydrocarbon chain wherein L² has at least one double bond and one or two additional methylene units of L² are optionally and independently replaced by —NRC(O)—, —C(O)NR—, —N(R)SO₂—, —SO₂N(R)—, —S—, —S(O)—, —SO₂—, —OC(O)—, —C(O)O—, cyclopropylene, —O—, —N(R)—, or —C(O)—; and Y is hydrogen or C₁₋₆ aliphatic optionally substituted with oxo, halogen, NO₂, or CN; or
(b) L² is a bivalent C₂₋₈ straight or branched, hydrocarbon chain wherein L² has at least one double bond and at least one methylene unit of L² is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L² are optionally and independently replaced by cyclopropylene, —O—, —N(R)—, or —C(O)—; and Y is hydrogen or C₁₋₆ aliphatic optionally substituted with oxo, halogen, NO₂, or CN; or
(c) L² is a bivalent C₂₋₈ straight or branched, hydrocarbon chain wherein L² has at least one double bond and at least one methylene unit of L² is replaced by —C(O)—, and one or two additional methylene units of L² are optionally and independently replaced by cyclopropylene, —O—, —N(R)—, or —C(O)—; and Y is hydrogen or C₁₋₆ aliphatic optionally substituted with oxo, halogen, NO₂, or CN; or
(d) L² is a bivalent C₂₋₈ straight or branched, hydrocarbon chain wherein L² has at least one double bond and at least one methylene unit of L² is replaced by —C(O)—; and Y is hydrogen or C₁₋₆ aliphatic optionally substituted with oxo, halogen, NO₂, or CN; or
(e) L² is a bivalent C₂₋₈ straight or branched, hydrocarbon chain wherein L² has at least one double bond and at least one methylene unit of L² is replaced by —OC(O)—; and Y is hydrogen or C₁₋₆ aliphatic optionally substituted with oxo, halogen, NO₂, or CN; or
(f) L² is —NRC(O)CH═CH—, —NRC(O)CH═CHCH₂N(CH₃)—, —NRC(O)CH═CHCH₂O—, —CH₂NRC(O)CH═CH—, —NRSO₂CH═CH—, —NRSO₂CH═CHCH₂—, —NRC(O)(C═N₂)—, —NRC(O)(C═N₂)C(O)—, —NRC(O)CH═CHCH₂N(CH₃)—, —NRSO₂CH═CH—, —NRSO₂CH═CHCH₂—, —NRC(O)CH═CHCH₂O—, —NRC(O)C(═CH₂)CH₂—, —CH₂NRC(O)—, —CH₂NRC(O)CH═CH—, —CH₂CH₂NRC(O)—, or —CH₂NRC(O)cyclopropylene-; wherein R is H or optionally substituted C₁₋₆ aliphatic; and Y is hydrogen or C₁₋₆ aliphatic optionally substituted with oxo, halogen, NO₂, or CN; or
(g) L² is —NHC(O)CH═CH—, —NHC(O)CH═CHCH₂N(CH₃)—, —NHC(O)CH═CHCH₂O—, —CH₂NHC(O)CH═CH—, —NHSO₂CH═CH—, —NHSO₂CH═CHCH₂—, —NHC(O)(C═N₂)—, —NHC(O)(C═N₂)C(O)—, —NHC(O)CH═CHCH₂N(CH₃)—, —NHSO₂CH═CH—, —NHSO₂CH═CHCH₂—, —NHC(O)CH═CHCH₂O—, —NHC(O)C(═CH₂)CH₂—, —CH₂NHC(O)—, —CH₂NHC(O)CH═CH—, —CH₂CH₂NHC(O)—, or —CH₂NHC(O)cyclopropylene-; and Y is hydrogen or C₁₋₆ aliphatic optionally substituted with oxo, halogen, NO₂, or CN; or
(h) L² is a bivalent C₂₋₈ straight or branched, hydrocarbon chain wherein L² has at least one alkylidenyl double bond and at least one methylene unit of L² is replaced by —C(O)—, —NRC(O)—, —C(O)NR—, —N(R)SO₂—, —SO₂N(R)—, —S—, —S(O)—, —SO₂—, —OC(O)—, or —C(O)O—, and one or two additional methylene units of L² are optionally and independently replaced by cyclopropylene, —O—, —N(R)—, or —C(O)—; and Y is hydrogen or C₁₋₆ aliphatic optionally substituted with oxo, halogen, NO₂, or CN; or
(i) L² is a bivalent C₂₋₈ straight or branched, hydrocarbon chain wherein L² has at least one triple bond and one or two additional methylene units of L² are optionally and independently replaced by —NRC(O)—, —C(O)NR—, —N(R)SO₂—, —SO₂N(R)—, —S—, —S(O)—, —SO₂—, —OC(O)—, or —C(O)O—, and Y is hydrogen or C₁₋₆ aliphatic optionally substituted with oxo, halogen, NO₂, or CN; or
(j) L² is —C≡C—, —C≡CCH₂N(isopropyl)-, —NHC(O)C≡CCH₂CH₂—, —CH₂—C≡C≡CH₂—, —C≡CCH₂O—, —CH₂C(O)C≡C—, —C(O)C≡C—, or —CH₂C(═O)C≡C—; and Y is hydrogen or C₁₋₆ aliphatic optionally substituted with oxo, halogen, NO₂, or CN; or
(k) L² is a bivalent C₂₋₈ straight or branched, hydrocarbon chain wherein one methylene unit of L² is replaced by cyclopropylene and one or two additional methylene units of L² are independently replaced by —NRC(O)—, —C(O)NR—, —N(R)SO₂—, —SO₂N(R)—, —S—, —S(O)—, —SO₂—, —OC(O)—, or —C(O)O—; and Y is hydrogen or C₁₋₆ aliphatic optionally substituted with oxo, halogen, NO₂, or CN; or
(l) L² is a covalent bond and Y is selected from:
   (i) C₁₋₆ alkyl substituted with oxo, halogen, NO₂, or CN;
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

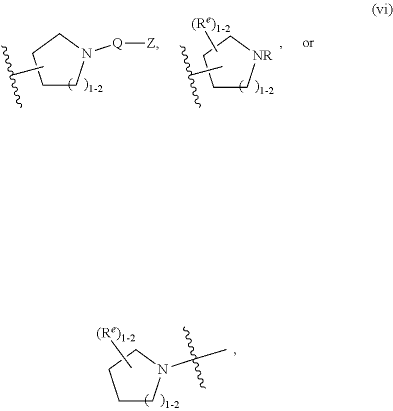

wherein each R, Q, Z, and Reis as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

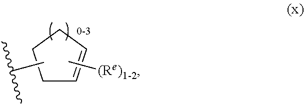

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

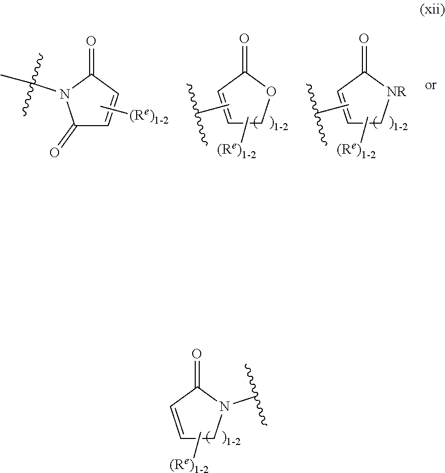

wherein each R and R^{e} is as defined above and described herein; or
(xii) a 6-membered aromatic ring having 0-2 nitrogens wherein said ring is substituted with 1-4 R^{e} groups, wherein each R^{e} group is as defined above and described herein; or

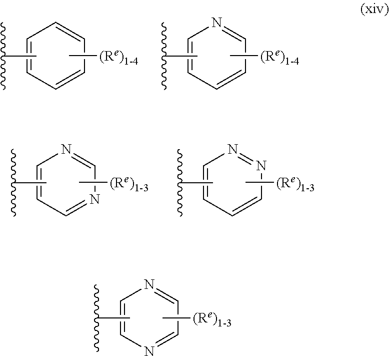

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

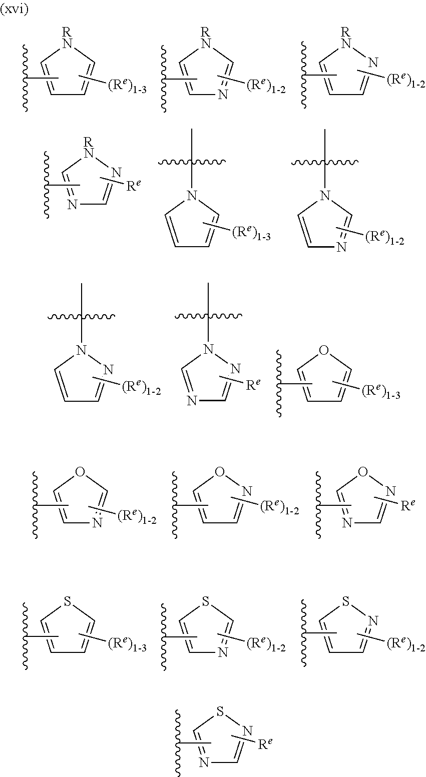

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(m) L² is —C(O)— and Y is selected from:
   (i) C₁₋₆ alkyl substituted with oxo, halogen, NO₂, or CN; or
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R groups, wherein each R^{e} is as defined above and described herein; or
   (v) a saturated 5-6 membered heterocyclic ring having 1-2 heteroatom selected from oxygen or nitrogen wherein said ring is substituted with 1-4 R groups, wherein each R^{e} is as defined above and described herein; or

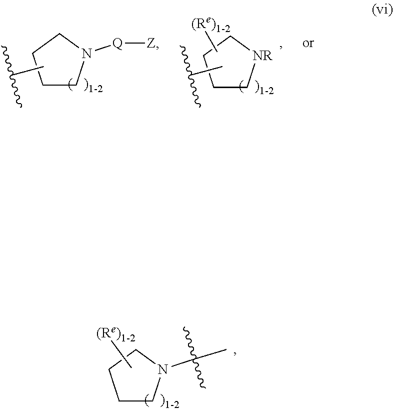

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each Reis as defined above and described herein; or
(ix) a partially unsaturated 3-6 membered carbocyclic ring, wherein said ring is substituted with 1-4 R^{e} groups, wherein each Reis as defined above and described herein; or

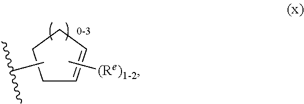

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each Reis as defined above and described herein; or

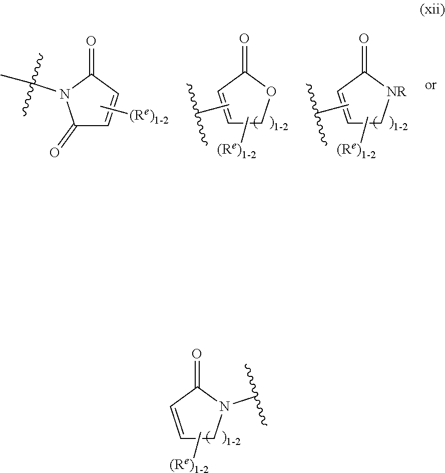

wherein each R and R^{e} is as defined above and described herein; or
(xiii) a 6-membered aromatic ring having 0-2 nitrogens wherein said ring is substituted with 1-4 R^{e} groups, wherein each R group is as defined above and described herein; or

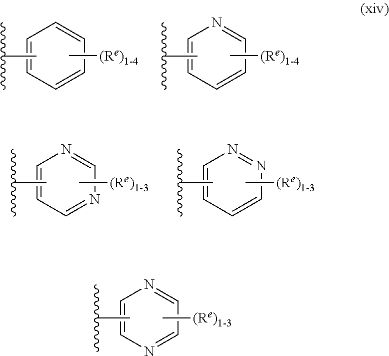

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

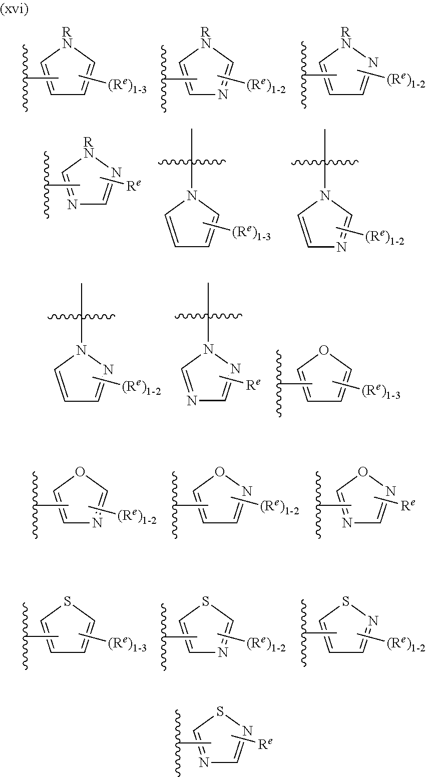

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(n) L² is —N(R)C(O)— and Y is selected from:
   (i) C₁₋₆ alkyl substituted with oxo, halogen, NO₂, or CN; or
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

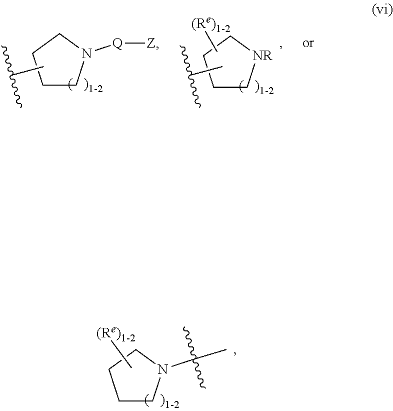

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

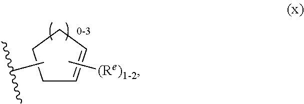

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

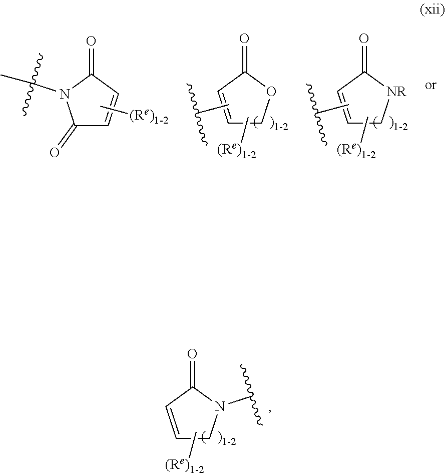

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

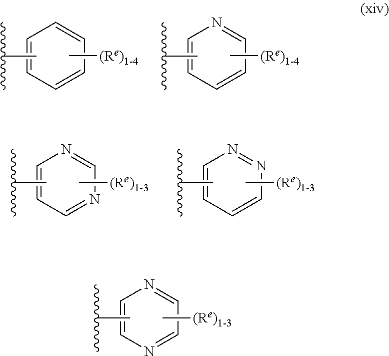

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

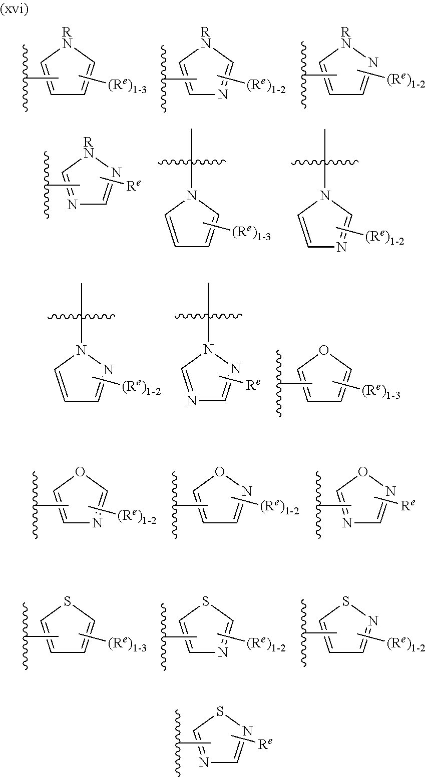

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(o) L² is a bivalent C₁₋₈ saturated or unsaturated, straight or branched, hydrocarbon chain; and Y is selected from:
   (i) C₁₋₆ alkyl substituted with oxo, halogen, NO₂, or CN;
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

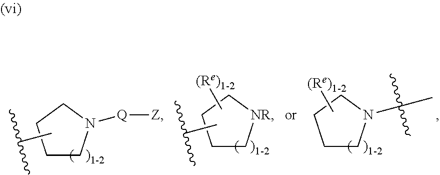

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

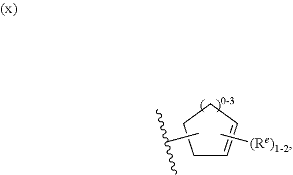

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

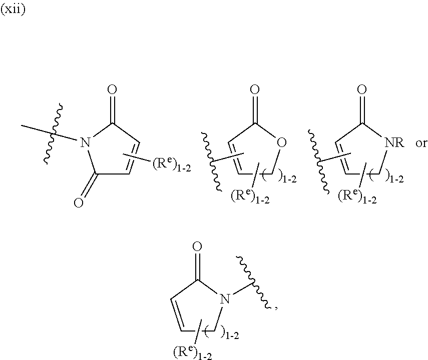

wherein
   each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

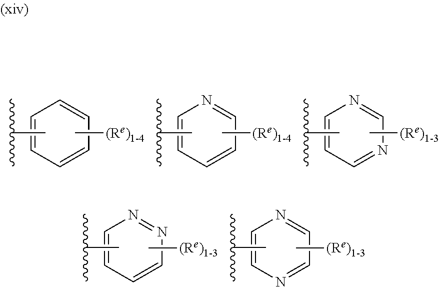

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

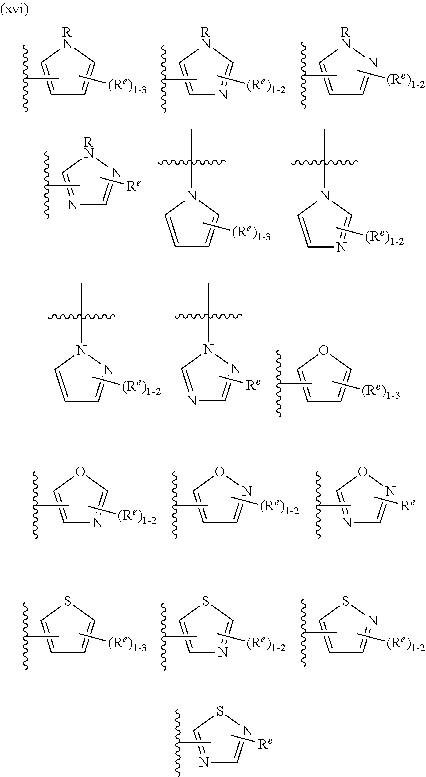

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein;
(p) L² is a covalent bond, —CH₂—, —NH—, —C(O)—, —CH₂NH—, —NHCH₂—, —NHC(O)—, —NHC(O)CH₂OC(O)—, —CH₂NHC(O)—, —NHSO₂—, —NHSO₂CH₂—, —NHC(O)CH₂OC(O)—, or —SO₂NH—; and Y is selected from:
   (i) C₁₋₆ alkyl substituted with oxo, halogen, NO₂, or CN; or
   (ii) C₂₋₆alkenyl optionally substituted with oxo, halogen, NO₂, or CN; or
   (iii) C₂₋₆alkynyl optionally substituted with oxo, halogen, NO₂, or CN; or
   (iv) a saturated 3-4 membered heterocyclic ring having 1 heteroatom selected from oxygen or nitrogen wherein said ring is substituted with 1-2 R^{e} groups, wherein each R^{e} is as defined above and described herein; or
   (v) a saturated 5-6 membered heterocyclic ring having 1-2 heteroatom selected from oxygen or nitrogen wherein said ring is substituted with 1-4 R^{e} groups, wherein each R^{e} is as defined above and described herein; or

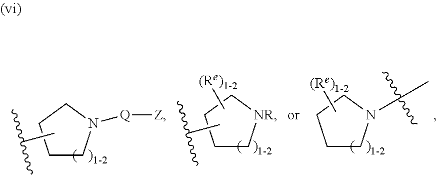

wherein each R, Q, Z, and R^{e} is as defined above and described herein; or
(vii) a saturated 3-6 membered carbocyclic ring, wherein said ring is substituted with 1-4 R^{e} groups, wherein each R^{e} is as defined above and described herein; or
(viii) a partially unsaturated 3-6 membered monocyclic ring having 0-3 heteroatoms independently selected from nitrogen, oxygen, or sulfur, wherein said ring is substituted with 1-4 R^{e} groups, wherein each R^{e} is as defined above and described herein; or
(ix) a partially unsaturated 3-6 membered carbocyclic ring, wherein said ring is substituted with 1-4 R^{e} groups, wherein each R^{e} is as defined above and described herein; or

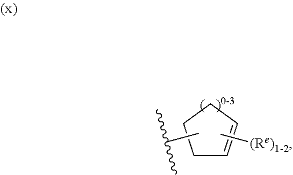

wherein each R^{e} is as defined above and described herein; or
(xi) a partially unsaturated 4-6 membered heterocyclic ring having 1-2 heteroatoms independently selected from nitrogen, oxygen, or sulfur, wherein said ring is substituted with 1-4 R^{e} groups, wherein each R^{e} is as defined above and described herein; or

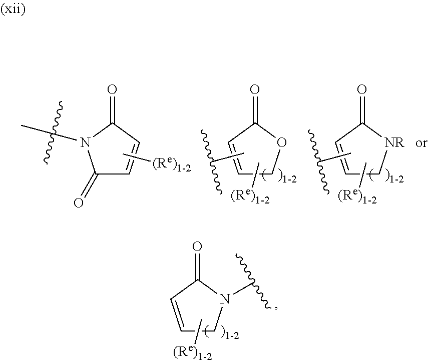

wherein each R and R^{e} is as defined above and described herein; or
(xiii) a 6-membered aromatic ring having 0-2 nitrogens wherein said ring is substituted with 1-4 R^{e} groups, wherein each R^{e} group is as defined above and described herein; or

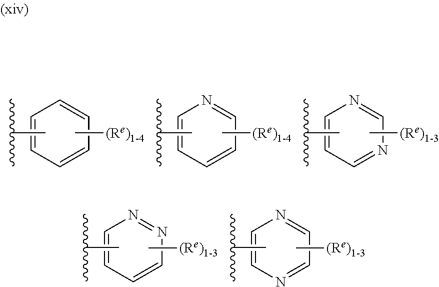

wherein each R^{e} is as defined above and described herein; or
(xv) a 5-membered heteroaryl ring having 1-3 heteroatoms independently selected from nitrogen, oxygen, or sulfur, wherein said ring is substituted with 1-3 R^{e} groups, wherein each R^{e} group is as defined above and described herein; or

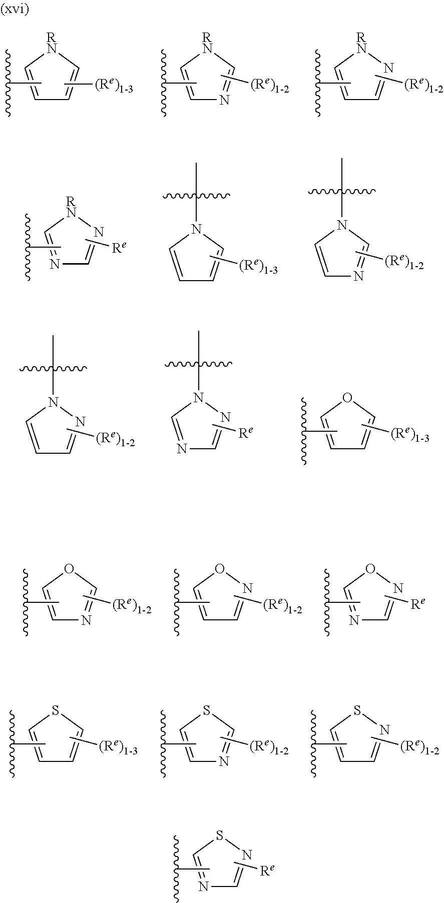

wherein each R and R^{e} is as defined above and described herein; or
(xvii) an 8-10 membered bicyclic, saturated, partially unsaturated, or aryl ring having 0-3 heteroatoms independently selected from nitrogen, oxygen, or sulfur, wherein said ring is substituted with 1-4 R^{e} groups, wherein R^{e} is as defined above and described herein.

In certain embodiments, the Y group is selected from those set forth in Table 3A below, wherein each wavy line indicates the point of attachment to the rest of the molecule.

TABLE 3A
Exemplary Y groups

| | |
|---|---|
| 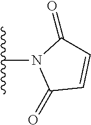 [SMILES: *N1C(=O)C=CC1=O] | a |
| 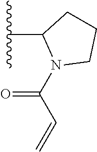 [SMILES: *C1CCCN1C(=O)C=C] | b |
| 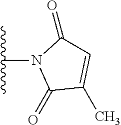 [SMILES: *N1C(=O)C=C(C)C1=O] | c |
| 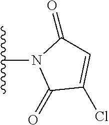 [SMILES: *N1C(=O)C=C(Cl)C1=O] | d |
| 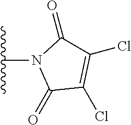 [SMILES: *N1C(=O)C(Cl)=C(Cl)C1=O] | e |
| 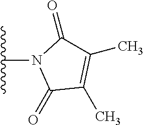 [SMILES: *N1C(=O)C(C)=C(C)C1=O] | f |
| 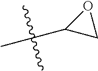 [SMILES: *C1CO1] | g |
| 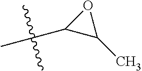 [SMILES: *C1OC1C] | h |
| 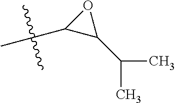 [SMILES: *C1OC1C(C)C] | i |
| 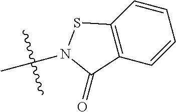 [SMILES: *n1sc2ccccc2c1=O] | j |
| 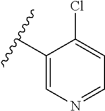 [SMILES: *c1cnccc1Cl] | k |
| 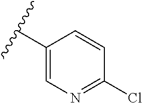 [SMILES: *c1ccc(Cl)nc1] | l |
| 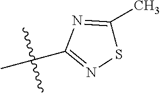 [SMILES: *c1nsc(C)n1] | m |
| 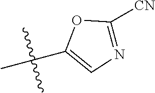 [SMILES: *c1cnc(C#N)o1] | n |
| 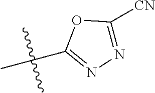 [SMILES: *c1nnc(C#N)o1] | o |
| 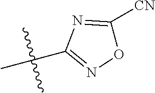 [SMILES: *c1noc(C#N)n1] | p |
| 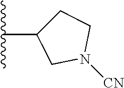 [SMILES: *C1CCN(C#N)C1] | q |
| 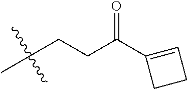 [SMILES: *CCC(=O)C1=CCC1] | r |
| 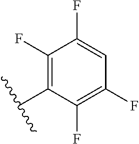 [SMILES: *c1c(F)c(F)cc(F)c1F] | s |
| 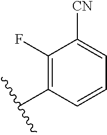 [SMILES: *c1cccc(C#N)c1F] | t |
| 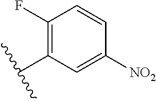 [SMILES: *c1cc([N+](=O)[O-])ccc1F] | u |
| 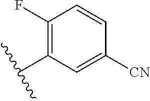 [SMILES: *c1cc(C#N)ccc1F] | v |
| 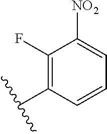 [SMILES: *c1cccc([N+](=O)[O-])c1F] | w |
| 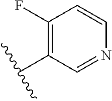 [SMILES: *c1cnccc1F] | x |
| 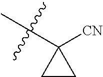 [SMILES: *C1(C#N)CC1] | y |
| 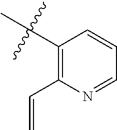 [SMILES: *c1cccnc1C=C] | z |
| 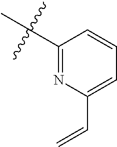 [SMILES: *c1cccc(C=C)n1] | aa |
| 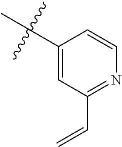 [SMILES: *c1ccnc(C=C)c1] | bb |
| 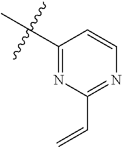 [SMILES: *c1ccnc(C=C)n1] | cc |
| 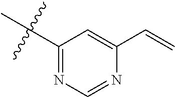 [SMILES: *c1cc(C=C)ncn1] | dd |
| 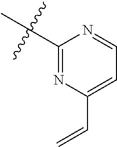 [SMILES: *c1nccc(C=C)n1] | ee |
| 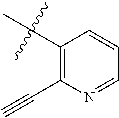 [SMILES: *c1cccnc1C#C] | ff |
| 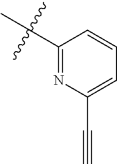 [SMILES: *c1cccc(C#C)n1] | gg |
| 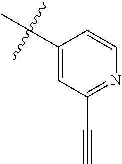 [SMILES: *c1ccnc(C#C)c1] | hh |
| 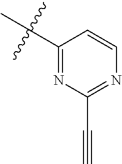 [SMILES: *c1ccnc(C#C)n1] | ii |
| 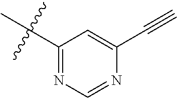 [SMILES: *c1cc(C#C)ncn1] | jj |
| 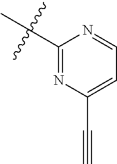 [SMILES: *c1nccc(C#C)n1] | kk |
| 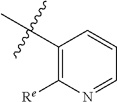 | ll |
| 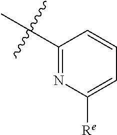 | mm |
| 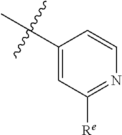 | nn |
| 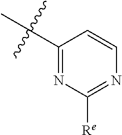 | oo |
| 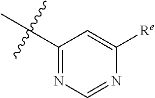 | pp |
| 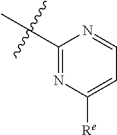 | qq |
| 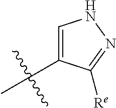 | rr |
| 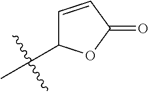 [SMILES: *C1C=CC(=O)O1] | ss |
| 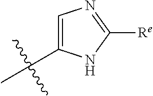 | tt |
| 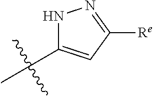 | uu |
| 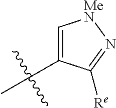 | vv |
| 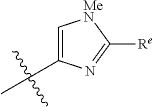 | ww |
| 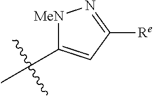 | xx |
| 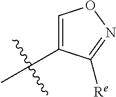 | yy |
| 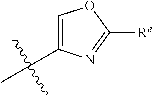 | zz |
| 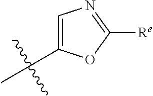 | aaa |
| 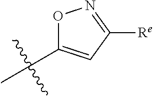 | bbb |
| 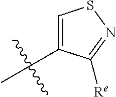 | ccc |
| 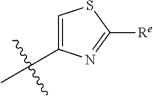 | ddd |
| 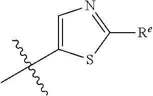 | eee |
| 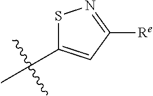 | fff |
| 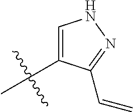 [SMILES: *c1c[nH]nc1C=C] | ggg |
| 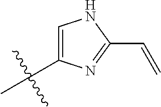 [SMILES: *c1c[nH]c(C=C)n1] | hhh |
| 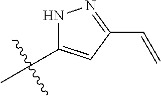 [SMILES: *c1cc(C=C)n[nH]1] | iii |
| 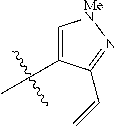 [SMILES: *c1cn(C)nc1C=C] | jjj |
| 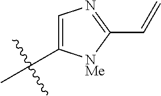 [SMILES: *c1cnc(C=C)n1C] | kkk |
| 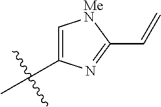 [SMILES: *c1cn(C)c(C=C)n1] | lll |
| 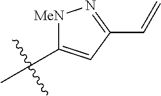 [SMILES: *c1cc(C=C)nn1C] | mmm |
| 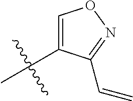 [SMILES: *c1conc1C=C] | nnn |
| 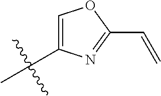 [SMILES: *c1coc(C=C)n1] | ooo |
| 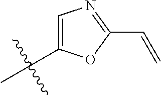 [SMILES: *c1cnc(C=C)o1] | ppp |
| 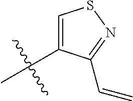 [SMILES: *c1csnc1C=C] | qqq |
| 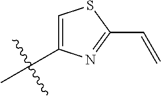 [SMILES: *c1csc(C=C)n1] | rrr |
| 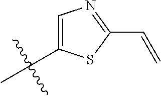 [SMILES: *c1cnc(C=C)s1] | sss |
| 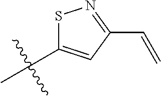 [SMILES: *c1cc(C=C)ns1] | ttt |
| 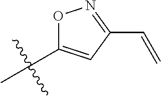 [SMILES: *c1cc(C=C)no1] | uuu |
| 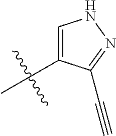 [SMILES: *c1c[nH]nc1C#C] | vvv |
| 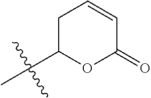 [SMILES: *C1CC=CC(=O)O1] | qqq |
| 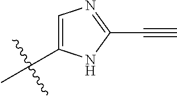 [SMILES: *c1cnc(C#C)[nH]1] | www |
| 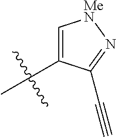 [SMILES: *c1cn(C)nc1C#C] | xxx |
| 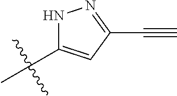 [SMILES: *c1cc(C#C)n[nH]1] | yyy |
| 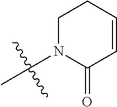 [SMILES: *N1CCC=CC1=O] | zzz |
| 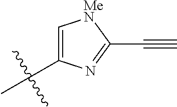 [SMILES: *c1cn(C)c(C#C)n1] | aaaa |
| 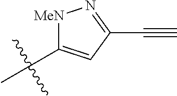 [SMILES: *c1cc(C#C)nn1C] | bbbb |
| 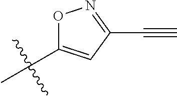 [SMILES: *c1cc(C#C)no1] | cccc |
| 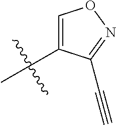 [SMILES: *c1conc1C#C] | dddd |
| 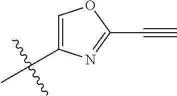 [SMILES: *c1coc(C#C)n1] | eeee |
| 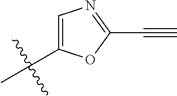 [SMILES: *c1cnc(C#C)o1] | ffff |
| 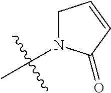 [SMILES: *N1CC=CC1=O] | gggg |
| 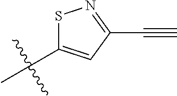 [SMILES: *c1cc(C#C)ns1] | hhhh |
| 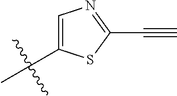 [SMILES: *c1cnc(C#C)s1] | iiii |
| 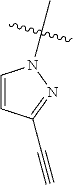 [SMILES: *n1ccc(C#C)n1] | jjjj |
| 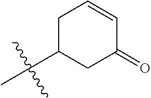 [SMILES: *C1CC=CC(=O)C1] | kkkk |
| 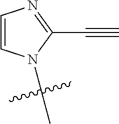 [SMILES: *n1ccnc1C#C] | llll |
| 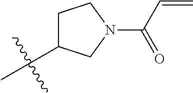 [SMILES: *C1CCN(C(=O)C=C)C1] | mmmm |
| 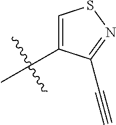 [SMILES: *c1csnc1C#C] | nnnn |
| 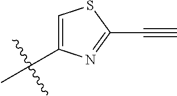 [SMILES: *c1csc(C#C)n1] | oooo |
| 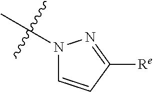 | pppp |
| 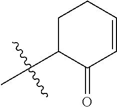 [SMILES: *C1CCC=CC1=O] | qqqq |
| 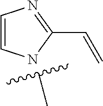 | rrrr |
| 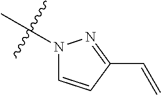 [SMILES: *n1ccc(C=C)n1] | ssss |
| 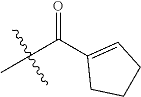 [SMILES: *C(=O)C1=CCCC1] | tttt |
| 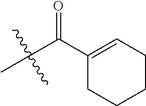 [SMILES: *C(=O)C1=CCCCC1] | uuuu |
| 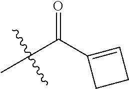 [SMILES: *C(=O)C1=CCC1] | vvvv |
| 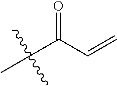 [SMILES: *C(=O)C=C] | wwww |
| 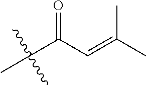 [SMILES: *C(=O)C=C(C)C] | xxxx |
| 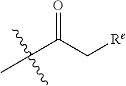 | yyyy |
| 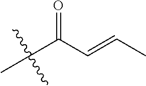 [SMILES: *C(=O)/C=C/C] | zzzz |
| 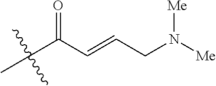 [SMILES: *C(=O)/C=C/CN(C)C] | aaaaa |
| 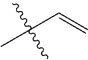 [SMILES: *C=C] | bbbbb |
| 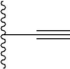 [SMILES: *C#C] | ccccc |

wherein each RN is independently a suitable leaving group, NO₂, CN or oxo.

In certain embodiments, a warhead group is —C≡CH, —C≡CCH₂NH(isopropyl), —NHC(O)C≡CCH₂CH₃, —CH₂≡C≡CH₃, —C≡CCH₂OH, —CH₂C(O)C≡CH, —C(O)C≡CH, or —CH₂C(═O)C≡CH. In some embodiments, R¹ is selected from —NHC(O)CH═CH₂, NHC(O)CH═CHCH₂N(CH₃)₂, or —CH₂NHC(O)CH═CH₂.

In certain embodiments, a warhead group is selected from those set forth in Table 3B, below, wherein each wavy line indicates the point of attachment to the rest of the molecule.

TABLE 3B
Exemplary Warhead Groups

| | |
|---|---|
| 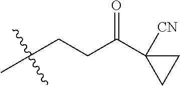 [SMILES: *CCC(=O)C1(C#N)CC1] | a |
| 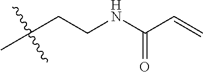 [SMILES: *CCNC(=O)C=C] | b |
| 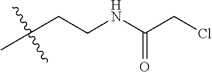 [SMILES: *CCNC(=O)CCl] | c |
| 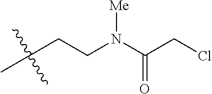 [SMILES: *CCN(C)C(=O)CCl] | d |
| 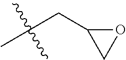 [SMILES: *CC1CO1] | e |
| 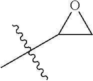 [SMILES: *C1CO1] | f |
| 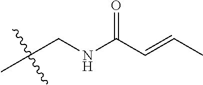 [SMILES: *CNC(=O)/C=C/C] | g |
| 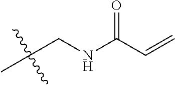 [SMILES: *CNC(=O)C=C] | h |
| 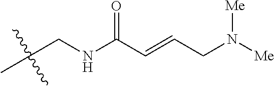 [SMILES: *CNC(=O)/C=C/CN(C)C] | i |
| 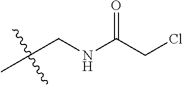 [SMILES: *CNC(=O)CCl] | j |
| 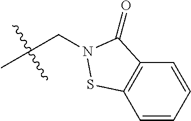 [SMILES: *Cn1sc2ccccc2c1=O] | k |
| 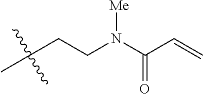 [SMILES: *CCN(C)C(=O)C=C] | l |
| 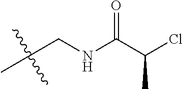 [SMILES: *CNC(=O)[C@H](C)Cl] | m |
| 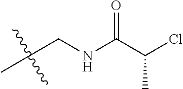 [SMILES: *CNC(=O)[C@@H](C)Cl] | n |
| 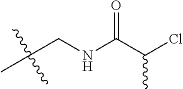 [SMILES: *CNC(=O)C(C)Cl] | o |
| 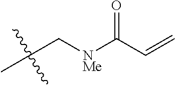 [SMILES: *CN(C)C(=O)C=C] | p |
| 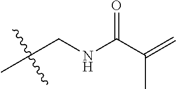 [SMILES: *CNC(=O)C(=C)C] | q |
| 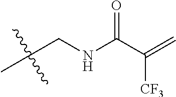 [SMILES: *CNC(=O)C(=C)C(F)(F)F] | r |
| 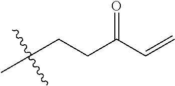 [SMILES: *CCC(=O)C=C] | s |
| 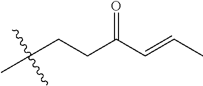 [SMILES: *CCC(=O)/C=C/C] | t |
| 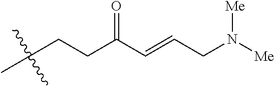 [SMILES: *CCC(=O)/C=C/CN(C)C] | u |
| 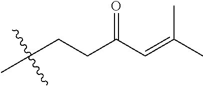 [SMILES: *CCC(=O)C=C(C)C] | v |
| 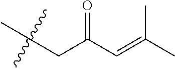 [SMILES: *CC(=O)C=C(C)C] | w |
| 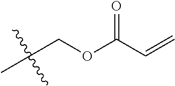 [SMILES: *COC(=O)C=C] | x |
| 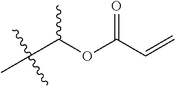 [SMILES: *C(C)OC(=O)C=C] | y |
| 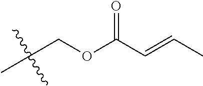 [SMILES: *COC(=O)/C=C/C] | z |
| 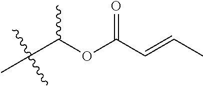 [SMILES: *C(C)OC(=O)/C=C/C] | aa |
| 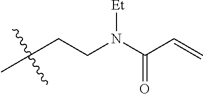 [SMILES: *CCN(CC)C(=O)C=C] | bb |
| 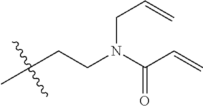 [SMILES: *CCN(CC=C)C(=O)C=C] | cc |
| 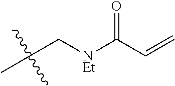 | dd |
| 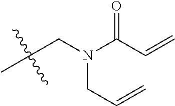 [SMILES: *CN(CC=C)C(=O)C=C] | ee |
| 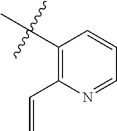 [SMILES: *c1cccnc1C=C] | ff |
| 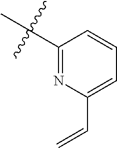 [SMILES: *c1cccc(C=C)n1] | gg |
| 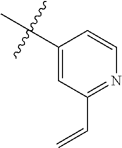 [SMILES: *c1ccnc(C=C)c1] | hh |
| 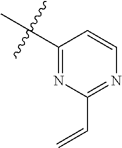 [SMILES: *c1ccnc(C=C)n1] | ii |
| 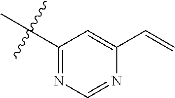 [SMILES: *c1cc(C=C)ncn1] | jj |
| 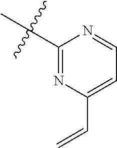 [SMILES: *c1nccc(C=C)n1] | kk |
| 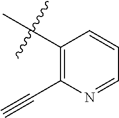 [SMILES: *c1cccnc1C#C] | ll |
| 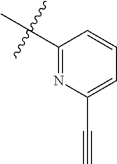 [SMILES: *c1cccc(C#C)n1] | mm |
| 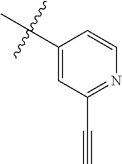 [SMILES: *c1ccnc(C#C)c1] | nn |
| 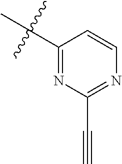 [SMILES: *c1ccnc(C#C)n1] | oo |
| 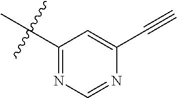 [SMILES: *c1cc(C#C)ncn1] | pp |
| 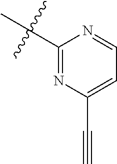 [SMILES: *c1nccc(C#C)n1] | qq |
| 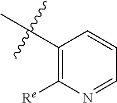 | rr |
| 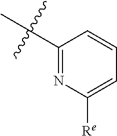 | ss |
| 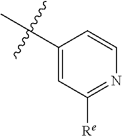 | tt |
| 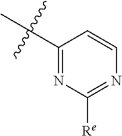 | uu |
| 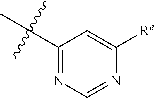 | vv |
| 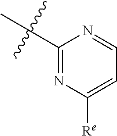 | ww |
| 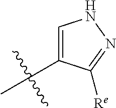 | xx |
| 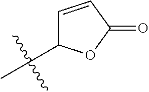 [SMILES: *C1C=CC(=O)O1] | yy |
| 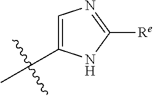 | zz |
| 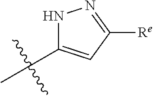 | aaa |
| 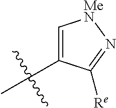 | bbb |
| 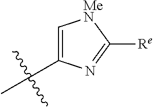 | ccc |
| 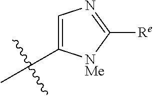 | ddd |
| 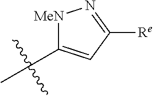 | eee |
| 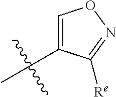 | fff |
| 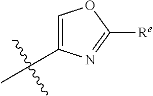 | ggg |
| 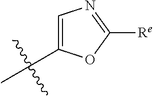 | hhh |
| 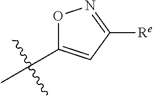 | iii |
| 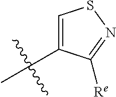 | jjj |
| 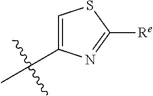 | kkk |
| 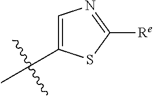 | lll |
| 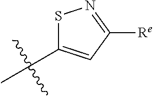 | mmm |
| 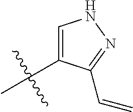 [SMILES: *c1c[nH]nc1C=C] | nnn |
| 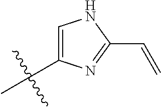 [SMILES: *c1c[nH]c(C=C)n1] | ooo |
| 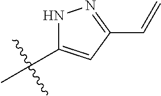 [SMILES: *c1cc(C=C)n[nH]1] | ppp |
| 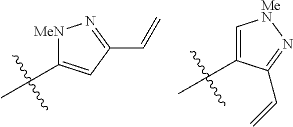 | qqq |
| 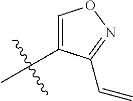 [SMILES: *c1conc1C=C] | rrr |
| 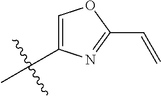 [SMILES: *c1coc(C=C)n1] | sss |
| 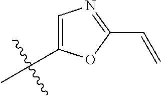 [SMILES: *c1cnc(C=C)o1] | ttt |
| 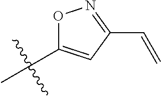 [SMILES: *c1cc(C=C)no1] | uuu |
| 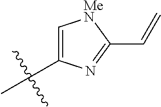 [SMILES: *c1cn(C)c(C=C)n1] | vvv |
| 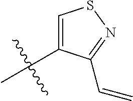 [SMILES: *c1csnc1C=C] | www |
| 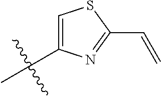 [SMILES: *c1csc(C=C)n1] | xxx |
| 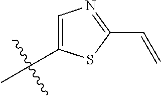 [SMILES: *c1cnc(C=C)s1] | yyy |
| 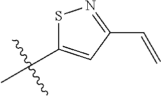 [SMILES: *c1cc(C=C)ns1] | zzz |
| 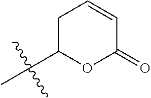 [SMILES: *C1CC=CC(=O)O1] | aaaa |
| 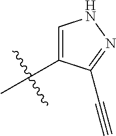 [SMILES: *c1c[nH]nc1C#C] | bbbb |
| 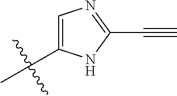 [SMILES: *c1cnc(C#C)[nH]1] | cccc |
| 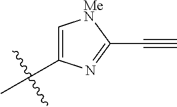 [SMILES: *c1cn(C)c(C#C)n1] | dddd |
| 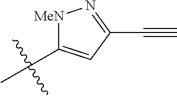 [SMILES: *c1cc(C#C)nn1C] | eeee |
| 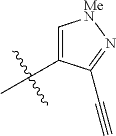 [SMILES: *c1cn(C)nc1C#C] | ffff |
| 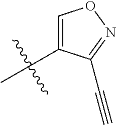 [SMILES: *c1conc1C#C] | gggg |
| 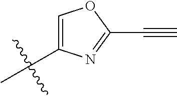 [SMILES: *c1coc(C#C)n1] | hhhh |
| 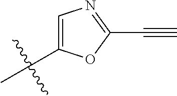 [SMILES: *c1cnc(C#C)o1] | iiii |
| 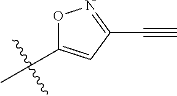 [SMILES: *c1cc(C#C)no1] | jjjj |
| 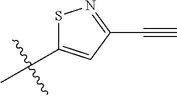 [SMILES: *c1cc(C#C)ns1] | kkkk |
| 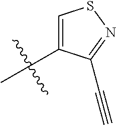 [SMILES: *c1csnc1C#C] | llll |
| 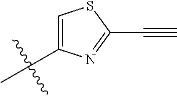 [SMILES: *c1csc(C#C)n1] | mmmm |
| 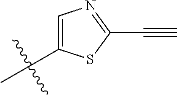 [SMILES: *c1cnc(C#C)s1] | nnnn |
| 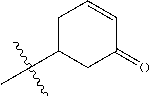 [SMILES: *C1CC=CC(=O)C1] | oooo |
| 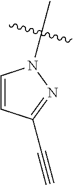 [SMILES: *n1ccc(C#C)n1] | pppp |
| 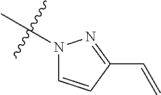 [SMILES: *n1ccc(C=C)n1] | qqqq |
| 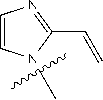 | rrrr |
| 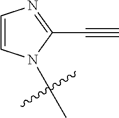 [SMILES: *n1ccnc1C#C] | ssss |
| 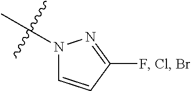 | tttt |
| 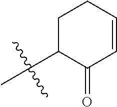 [SMILES: *C1CCC=CC1=O] | uuuu |
| 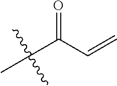 [SMILES: *C(=O)C=C] | vvvv |
| 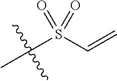 [SMILES: *S(=O)(=O)C=C] | wwww |
| 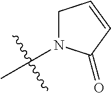 [SMILES: *N1CC=CC1=O] | xxxx |
| 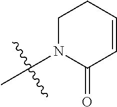 [SMILES: *N1CCC=CC1=O] | yyyy |
| 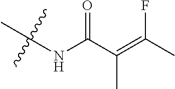 [SMILES: *NC(=O)/C(C)=C(/C)F] | zzzz |
| 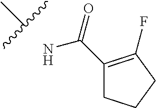 | aaaaa |
| 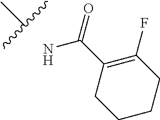 | bbbbb |
| 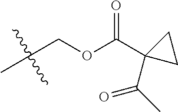 [SMILES: *COC(=O)C1(C(C)=O)CC1] | ccccc |
| 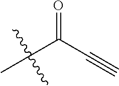 [SMILES: *C(=O)C#C] | ddddd |
| 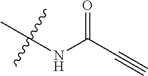 [SMILES: *NC(=O)C#C] | eeeee |
| 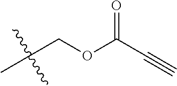 [SMILES: *COC(=O)C#C] | fffff |
| 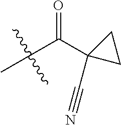 [SMILES: *C(=O)C1(C#N)CC1] | ggggg |
| 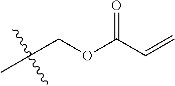 [SMILES: *COC(=O)C=C] | hhhhh |
| 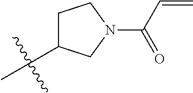 [SMILES: *C1CCN(C(=O)C=C)C1] | iiiii |
| 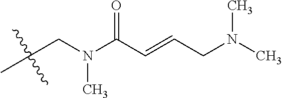 [SMILES: *CN(C)C(=O)/C=C/CN(C)C] | jjjjj |
| 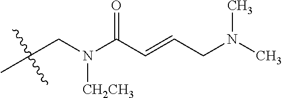 [SMILES: *CN(CC)C(=O)/C=C/CN(C)C] | kkkkk |
| 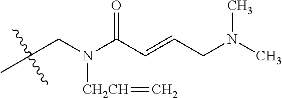 | lllll |
| 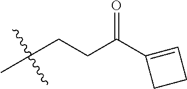 [SMILES: *CCC(=O)C1=CCC1] | mmmmm |
| 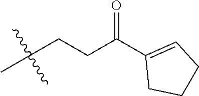 [SMILES: *CCC(=O)C1=CCCC1] | nnnnn |
| 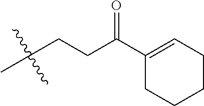 [SMILES: *CCC(=O)C1=CCCCC1] | ooooo |
| 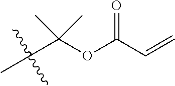 [SMILES: *C(C)(C)OC(=O)C=C] | ppppp |
| 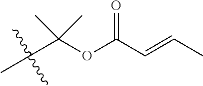 [SMILES: *C(C)(C)OC(=O)/C=C/C] | qqqqq |
| 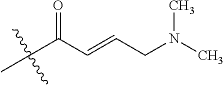 [SMILES: *C(=O)/C=C/CN(C)C] | rrrrr |
| 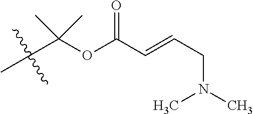 [SMILES: *C(C)(C)OC(=O)/C=C/CN(C)C] | sssss |
| 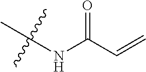 [SMILES: *NC(=O)C=C] | ttttt |
| 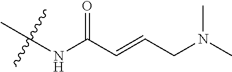 [SMILES: *NC(=O)/C=C/CN(C)C] | uuuuu |
| 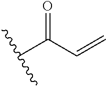 [SMILES: *C(=O)C=C] | vvvvv |
| 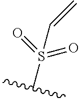 [SMILES: *S(=O)(=O)C=C] | wwwww |
| 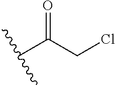 [SMILES: *C(=O)CCl] | xxxxx |
| 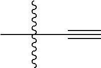 [SMILES: *C#C] | yyyyy |
| 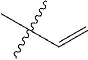 [SMILES: *C=C] | zzzzz |
| 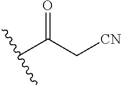 [SMILES: *C(=O)CC#N] | aaaaaa |
| 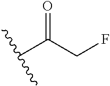 [SMILES: *C(=O)CF] | bbbbbb |
| 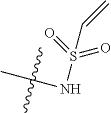 [SMILES: *NS(=O)(=O)C=C] | cccccc |
| 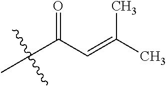 [SMILES: *C(=O)C=C(C)C] | dddddd |
| 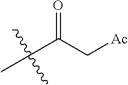 [SMILES: *C(=O)CC(C)=O] | eeeeee |
| 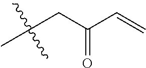 [SMILES: *CC(=O)C=C] | ffffff |
| 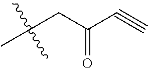 [SMILES: *CC(=O)C#C] | gggggg |
| 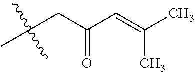 [SMILES: *CC(=O)C=C(C)C] | hhhhhh |
| 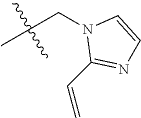 [SMILES: *Cn1ccnc1C=C] | iiiiii |
| 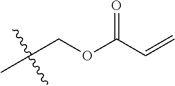 [SMILES: *COC(=O)C=C] | jjjjjj |
| 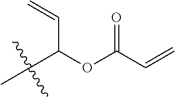 [SMILES: *C(C=C)OC(=O)C=C] | kkkkkk |
| 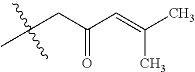 [SMILES: *CC(=O)C=C(C)C] | llllll |
| 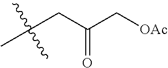 [SMILES: *CC(=O)COC(C)=O] | mmmmmm |
| 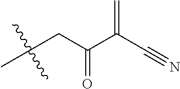 [SMILES: *CC(=O)C(=C)C#N] | nnnnnn |
| 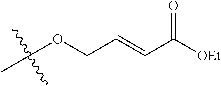 [SMILES: *OC/C=C/C(=O)OCC] | oooooo |
| 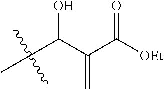 [SMILES: *C(O)C(=C)C(=O)OCC] | pppppp |
| 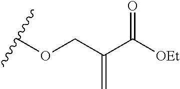 [SMILES: *OCC(=C)C(=O)OCC] | qqqqqq |
| 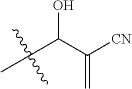 [SMILES: *C(O)C(=C)C#N] | rrrrrr |
| 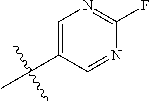 [SMILES: *c1cnc(F)nc1] | sssssss |
| 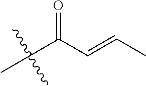 [SMILES: *C(=O)/C=C/C] | tttttt |
| 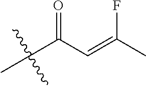 [SMILES: *C(=O)/C=C(/C)F] | uuuuuu |
| 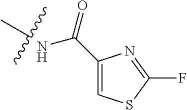 | vvvvvv |
| 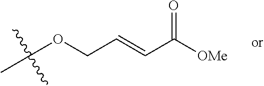 [SMILES: *OC/C=C/C(=O)OC] | wwwwww |
| 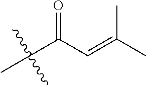 [SMILES: *C(=O)C=C(C)C] | xxxxxx |

wherein each R^{e} is independently a suitable leaving group, NO₂, CN, or oxo.

In some embodiments, Y of a warhead group is an isoxazoline compound or derivative capable of covalently binding to serine. In some embodiments, Y of a warhead group is an isoxazoline compound or derivative described in WO 2010135360, the entire content of which is incorporated herein by reference. As understood by one skilled in the art, an isoxazoline compound or derivative described in WO 2010135360, as Y of a warhead group, can covalently connect to L² of the warhead group at any reasonable position of the isoxazoline compound or derivative. In some embodiments, Y of a warhead group is:

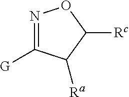

wherein G, R^{a}, and R^{c} are:

| G | R^{a} | R^{c} |
|---|---|---|
| —Br | —H | —H |
| —Cl | —H | —H |
| 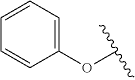 [SMILES: *Oc1ccccc1] | —H | —H |
| 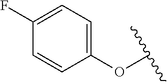 [SMILES: *Oc1ccc(F)cc1] | —H | —H |
| 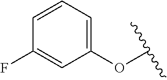 [SMILES: *Oc1cccc(F)c1] | —H | —H |
| 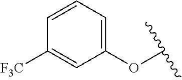 [SMILES: *Oc1cccc(C(F)(F)F)c1] | —H | —H |
| 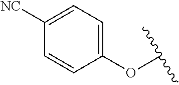 [SMILES: *Oc1ccc(C#N)cc1] | —H | —H |
| 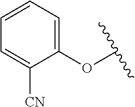 [SMILES: *Oc1ccccc1C#N] | —H | —H |
| 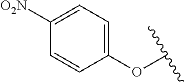 [SMILES: *Oc1ccc([N+](=O)[O-])cc1] | —H | —H |
| 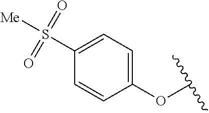 [SMILES: *Oc1ccc(S(C)(=O)=O)cc1] | —H | —H |
| 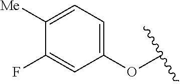 [SMILES: *Oc1ccc(C)c(F)c1] | —H | —H |
| 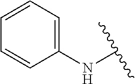 [SMILES: *Nc1ccccc1] | —H | —H |
| 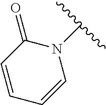 [SMILES: *n1ccccc1=O] | —H | —H |
| 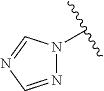 [SMILES: *n1cncn1] | —H | —H |
| 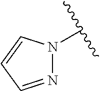 [SMILES: *n1cccn1] | —H | —H |
| 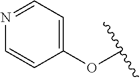 [SMILES: *Oc1ccncc1] | —H | —H |
| 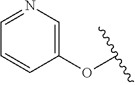 [SMILES: *Oc1cccnc1] | —H | —H |
| 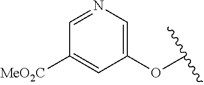 [SMILES: *Oc1cncc(C(=O)OC)c1] | —H | —H |
| 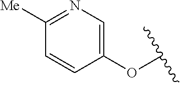 [SMILES: *Oc1ccc(C)nc1] | —H | —H |
| 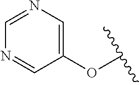 [SMILES: *Oc1cncnc1] | —H | —H |
| 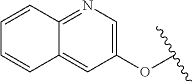 [SMILES: *Oc1cnc2ccccc2c1] | —H | —H |
| 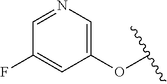 [SMILES: *Oc1cncc(F)c1] | —H | —H |
| 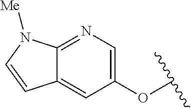 [SMILES: *Oc1cnc2c(ccn2C)c1] | —H | —H |
| —OMe | —H | —H |
| 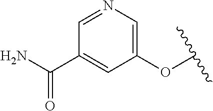 [SMILES: *Oc1cncc(C(N)=O)c1] | —H | —H |
| 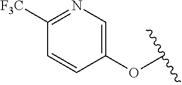 [SMILES: *Oc1ccc(C(F)(F)F)nc1] | —H | —H |
| 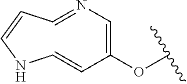 [SMILES: *Oc1cc[nH]cccnc1] | —H | —H |
| 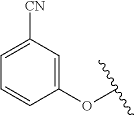 [SMILES: *Oc1cccc(C#N)c1] | —H | —H |
| 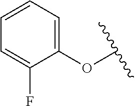 [SMILES: *Oc1ccccc1F] | —H | —H |
| 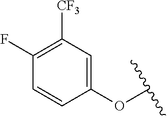 [SMILES: *Oc1ccc(F)c(C(F)(F)F)c1] | —H | —H |
| 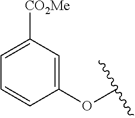 [SMILES: *Oc1cccc(C(=O)OC)c1] | —H | —H |
| 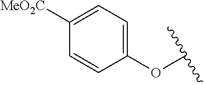 [SMILES: *Oc1ccc(C(=O)OC)cc1] | —H | —H |
| 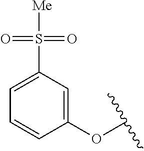 [SMILES: *Oc1cccc(S(C)(=O)=O)c1] | —H | —H |
| 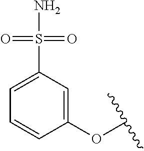 [SMILES: *Oc1cccc(S(N)(=O)=O)c1] | —H | —H |
| 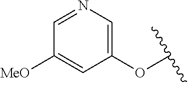 [SMILES: *Oc1cncc(OC)c1] | —H | —H |
| 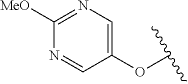 [SMILES: *Oc1cnc(OC)nc1] | —H | —H |
| 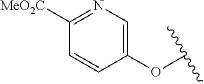 [SMILES: *Oc1ccc(C(=O)OC)nc1] | —H | —H |
| 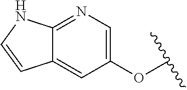 [SMILES: *Oc1cnc2[nH]ccc2c1] | —H | —H |
| 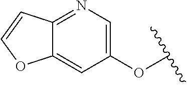 [SMILES: *Oc1cnc2ccoc2c1] | —H | —H |
| 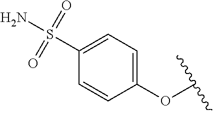 [SMILES: *Oc1ccc(S(N)(=O)=O)cc1] | —H | —H |
| 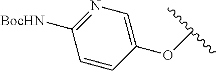 [SMILES: *Oc1ccc(NC(=O)OC(C)(C)C)nc1] | —H | —H |
| 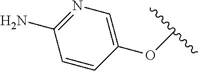 [SMILES: *Oc1ccc(N)nc1] | —H | —H |
| 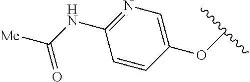 [SMILES: *Oc1ccc(NC(C)=O)nc1] | —H | —H |
| 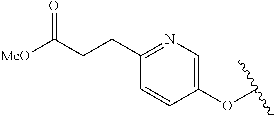 [SMILES: *Oc1ccc(CCC(=O)OC)nc1] | —H | —H |
| 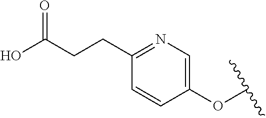 [SMILES: *Oc1ccc(CCC(=O)O)nc1] | —H | —H |
| 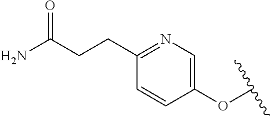 [SMILES: *Oc1ccc(CCC(N)=O)nc1] | —H | —H |
| 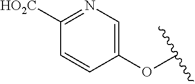 [SMILES: *Oc1ccc(C(=O)O)nc1] | —H | —H |
| 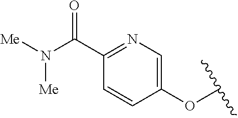 [SMILES: *Oc1ccc(C(=O)N(C)C)nc1] | —H | —H |
| 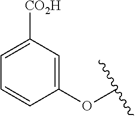 [SMILES: *Oc1cccc(C(=O)O)c1] | —H | —H |
| 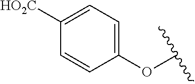 [SMILES: *Oc1ccc(C(=O)O)cc1] | —H | —H |
| 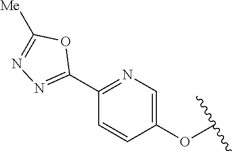 [SMILES: *Oc1ccc(-c2nnc(C)o2)nc1] | —H | —H |
| 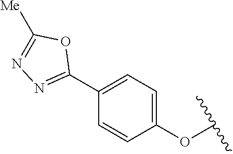 [SMILES: *Oc1ccc(-c2nnc(C)o2)cc1] | —H | —H |
| 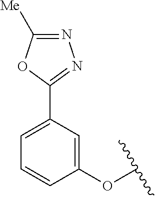 [SMILES: *Oc1cccc(-c2nnc(C)o2)c1] | —H | —H |
| 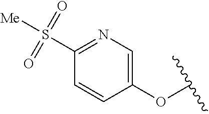 [SMILES: *Oc1ccc(S(C)(=O)=O)nc1] | —H | —H |
| 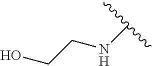 [SMILES: *NCCO] | —H | —H |
| 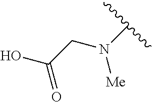 [SMILES: *N(C)CC(=O)O] | —H | —H |
| 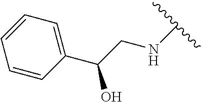 [SMILES: *NC[C@@H](O)c1ccccc1] | —H | —H |
| 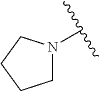 [SMILES: *N1CCCC1] | —H | —H |
| 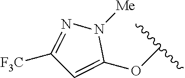 [SMILES: *Oc1cc(C(F)(F)F)nn1C] | —H | —H |
| 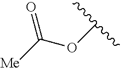 [SMILES: *OC(C)=O] | —H | —H |
| —Br | —CH₃ | —H |
| —Br | —CH₃ | —H |
| 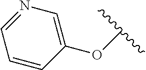 [SMILES: *Oc1cccnc1] | —CH₃ | —H |
| —Br | —H | —CH₃ |
| 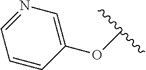 [SMILES: *Oc1cccnc1] | —H | —CH₃ |
| —Br | —H | —CF₃ |
| 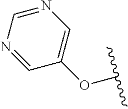 [SMILES: *Oc1cncnc1] | —H | —CF₃ |
| —Br | —H | —CH₂CH₃ |

Degradation Inducing Moiety (DIM)

In certain embodiments, the present invention provides a compound of formula I:

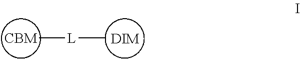

or a pharmaceutically acceptable salt thereof, wherein L and CBM are as described above and herein, and DIM is a degradation inducing moiety selected from LBM, a lysine mimetic, or a hydrogen atom.

In some embodiments, DIM is LBM as described above and herein. In some embodiments, DIM is a lysine mimetic. In some embodiments, the covalent attachment of ubiquitin to CDK2 protein is achieved through the action of a lysine mimetic. In some embodiments, upon the binding of a compound of formula I to CDK2 protein, the moiety that mimics a lysine undergoes ubiquitination thereby marking CDK2 protein for degradation via the Ubiquitin-Proteasome Pathway (UPP).

In some embodiments, DIM is

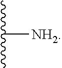 [SMILES: *N]

In some embodiments, DIM is

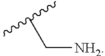 [SMILES: *CN]

In some embodiments, DIM is

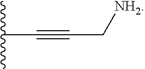 [SMILES: *C#CCN]

In some embodiments, DIM is selected from those depicted in Table 2, below.

In some embodiments, the present invention provides the compound of formula I as a compound of formula I-aaaa:

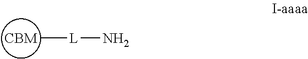

or a pharmaceutically acceptable salt thereof, wherein each of CBM and L is as defined above and described in embodiments herein, both singly and in combination.

In some embodiments, the present invention provides the compound of formula I as a compound of formula I-aaaa-1:

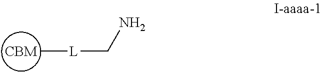

or a pharmaceutically acceptable salt thereof, wherein each of CBM and L is as defined above and described in embodiments herein, both singly and in combination.

In some embodiments, the present invention provides the compound of formula I as a compound of formula I-aaaa-2:

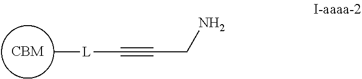

or a pharmaceutically acceptable salt thereof, wherein each of CBM and L is as defined above and described in embodiments herein, both singly and in combination.

In certain embodiments, the present invention provides a compound of Formula I, wherein DIM is a lysine mimetic

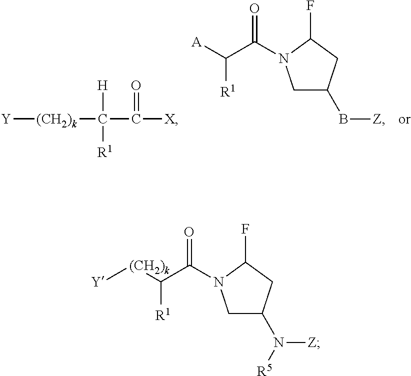

thereby forming a compound of Formulae I-bbbb-1, I-bbbb-2, or I-bbbb-3, respectively:

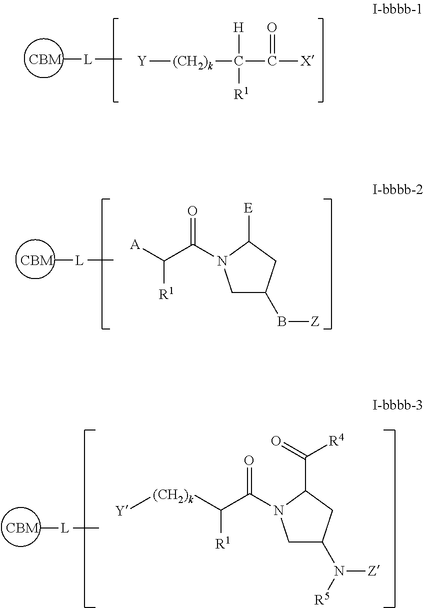

or a pharmaceutically acceptable salt thereof, wherein L and CBM are as defined above and described in embodiments herein, and wherein each of the variables R¹, R⁴, R⁵, A, B, E, Y, Y′, Z, Z′, and k are as defined and described in U.S. Pat. No. 7,622,496, the entirety of each of which is herein incorporated by reference.

Hydrogen Atom

In some embodiments, DIM is a hydrogen atom. In some embodiments, the covalent attachment of ubiquitin to CDK2 protein is achieved through a provided compound wherein DIM is a hydrogen atom. In some embodiments, upon the binding of a compound of formula I to CDK2 protein, the moiety being hydrogen effectuates ubiquitination thereby marking CDK2 protein for degradation via the Ubiquitin-Proteasome Pathway (UPP).

In some embodiments, DIM is selected from those depicted in Table 2, below.

In some embodiments, the present invention provides the compound of formula I wherein DIM is a hydrogen atom, thereby forming a compound of formula I-cccc:

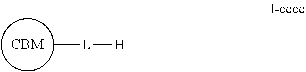

or a pharmaceutically acceptable salt thereof, wherein each of CBM and L is as defined above and described in embodiments herein, both singly and in combination.

Linker (L)

As defined above and described herein, L is a bivalent moiety that connects CBM to LBM or CBM to DIM.

In some embodiments, L is a bivalent moiety that connects CBM to LBM. In some embodiments, L is a bivalent moiety that connects CBM to DIM. In some embodiments, L is a bivalent moiety that connects CBM to a lysine mimetic.

In some embodiments, L is a covalent bond or a bivalent, saturated or partially unsaturated, straight or branched C₁₋₅₀ hydrocarbon chain, wherein 0-6 methylene units of L are independently replaced by -Cy-, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

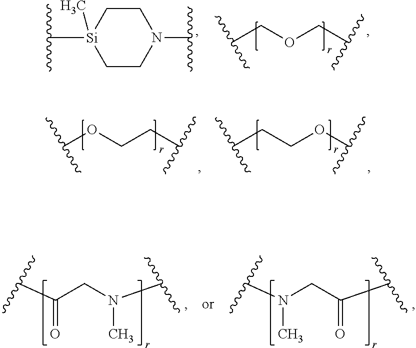

wherein:
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur,
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur, and;
r is 0, 1, 2, 3, 4, 5, 6, 7, 8, 9, or 10.

In some embodiments, L is substituted with deuterium.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 6-11 membered saturated or partially unsaturated spiro carbocyclylenyl. In some embodiments, each -Cy- is independently an optionally substituted 8-10 membered bicyclic saturated or partially unsaturated carbocyclylenyl. In some embodiments, each -Cy- is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 6-11 membered saturated or partially unsaturated spiro heterocyclylenyl having 1-2 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, and sulfur.

In some embodiments, -Cy- is substituted with one or more halogen, optionally substituted C₁₋₆ alkyl, or cyclopropylenyl.

In some embodiments, -Cy- is substituted with C₁₋₆ alkyl (e.g., methyl, ethyl, isopropyl). In some embodiments, -Cy- is substituted with methyl. In some embodiments, -Cy- is substituted with two methyls. In some embodiments, -Cy- is substituted with geminal dimethyl. In some embodiments, -Cy- is substituted with —CHF₂. In some embodiments, -Cy- is substituted with —CH₂OMe. In some embodiments, -Cy- is substituted with oxo. In some embodiments, -Cy- is substituted with halogen. In some embodiments, -Cy- is substituted with fluoro. In some embodiments, -Cy- is substituted with geminal difluoro. In some embodiments, -Cy- is substituted with —OH. In some embodiments, -Cy- is substituted with —NR₂.

In some embodiments, -Cy- is selected from those depicted in Table 2, below.

In some embodiments, r is 0. In some embodiments, r is 1. In some embodiments, r is 2. In some embodiments, r is 3. In some embodiments, r is 4. In some embodiments, r is 5. In some embodiments, r is 6. In some embodiments, r is 7. In some embodiments, r is 8. In some embodiments, r is 9. In some embodiments, r is 10.

In some embodiments, r is selected from those depicted in Table 2, below.

In some embodiments, L is —NR—(C₁₋₁₀ aliphatic)-. In some embodiments, L is —(C₁₋₁₀ aliphatic)-NR—(C₁₋₁₀ aliphatic)-. In some embodiments, L is —(C₁₋₁₀ aliphatic)-NR—(CH₂CH₂O)₁₋₁₀CH₂CH₂—. In some embodiments, L is -Cy-NR—(C₁₋₁₀ aliphatic)-. In some embodiments, L is -Cy-(C₁₋₁₀ aliphatic)-NR—. In some embodiments, L is -Cy-(C₁₋₁₀ aliphatic)-NR—(C₁₋₁₀ aliphatic)-. In some embodiments, L is —(C₁₋₁₀ aliphatic)-Cy-NR—(C₁₋₁₀ aliphatic)-. In some embodiments, L is —(C₁₋₁₀ aliphatic)-Cy-(C₁₋₁₀ aliphatic)-NR—. In some embodiments, L is —(C₁₋₁₀ aliphatic)-Cy-(C₁₋₁₀ aliphatic)-NR—(C₁₋₁₀ aliphatic)-. In some embodiments, L is -Cy-(C₁₋₁₀ aliphatic)-Cy-NR—. In some embodiments, L is -Cy-(C₁₋₁₀ aliphatic)-NR-Cy-. In some embodiments, L is -Cy-(C₁₋₁₀ aliphatic)-Cy-NR—(C₁₋₁₀ aliphatic)-. In some embodiments, L is -Cy-(C₁₋₁₀ aliphatic)-NR-Cy-(C₁₋₁₀ aliphatic)-.

In some embodiments, L is —CONR—(C₁₋₁₀ aliphatic)-. In some embodiments, L is —(C₁₋₁₀ aliphatic)-CONR—(C₁₋₁₀ aliphatic)-. In some embodiments, L is —(C₁₋₁₀ aliphatic)-CONR—(CH₂CH₂O)₁₋₁₀CH₂CH₂—. In some embodiments, L is -Cy-CONR—(C₁₋₁₀ aliphatic)-. In some embodiments, L is -Cy-(C₁₋₁₀ aliphatic)-CONR—. In some embodiments, L is -Cy-(C₁₋₁₀ aliphatic)-CONR—(C₁₋₁₀ aliphatic)-. In some embodiments, L is —(C₁₋₁₀ aliphatic)-Cy-CONR—(C₁₋₁₀ aliphatic)-. In some embodiments, L is —(C₁₋₁₀ aliphatic)-Cy-(C₁₋₁₀ aliphatic)-CONR—. In some embodiments, L is —(C₁₋₁₀ aliphatic)-Cy-(C₁₋₁₀ aliphatic)-CONR—(C₁₋₁₀ aliphatic)-. In some embodiments, L is -Cy-(C₁₋₁₀ aliphatic)-Cy-CONR—. In some embodiments, L is -Cy-(C₁₋₁₀ aliphatic)-CONR-Cy-. In some embodiments, L is -Cy-(C₁₋₁₀ aliphatic)-Cy-CONR—(C₁₋₁₀ aliphatic)-. In some embodiments, L is -Cy-(C₁₋₁₀ aliphatic)-CONR-Cy-(C₁₋₁₀ aliphatic)-.

In some embodiments, L is —NRCO—(C₁₋₁₀ aliphatic)-. In some embodiments, L is —(C₁₋₁₀ aliphatic)-NRCO—(C₁₋₁₀ aliphatic)-. In some embodiments, L is —(C₁₋₁₀ aliphatic)-NRCO—(CH₂CH₂O)₁₋₁₀CH₂CH₂—. In some embodiments, L is -Cy-NRCO—(C₁₋₁₀ aliphatic)-. In some embodiments, L is -Cy-(C₁₋₁₀ aliphatic)-NRCO—. In some embodiments, L is -Cy-(C₁₋₁₀ aliphatic)-NRCO—(C₁₋₁₀ aliphatic)-. In some embodiments, L is —(C₁₋₁₀ aliphatic)-Cy-NRCO—(C₁₋₁₀ aliphatic)-. In some embodiments, L is —(C₁₋₁₀ aliphatic)-Cy-(C₁₋₁₀ aliphatic)-NRCO—. In some embodiments, L is —(C₁₋₁₀ aliphatic)-Cy-(C₁₋₁₀ aliphatic)-NRCO—(C₁₋₁₀ aliphatic)-. In some embodiments, L is -Cy-(C₁₋₁₀ aliphatic)-Cy-NRCO—. In some embodiments, L is -Cy-(C₁₋₁₀ aliphatic)-NRCO-Cy-. In some embodiments, L is -Cy-(C₁₋₁₀ aliphatic)-Cy-NRCO—(C₁₋₁₀ aliphatic)-. In some embodiments, L is -Cy-(C₁₋₁₀ aliphatic)-NRCO-Cy-(C₁₋₁₀ aliphatic)-.

In some embodiments, L is —O—(C₁₋₁₀ aliphatic)-. In some embodiments, L is —(C₁₋₁₀ aliphatic)-O—(C₁₋₁₀ aliphatic)-. In some embodiments, L is —(C₁₋₁₀ aliphatic)-O—(CH₂CH₂O)₁₋₁₀CH₂CH₂—. In some embodiments, L is -Cy-O—(C₁₋₁₀ aliphatic)-. In some embodiments, L is -Cy-(C₁₋₁₀ aliphatic)-O—. In some embodiments, L is -Cy-(C₁₋₁₀ aliphatic)-O—(C₁₋₁₀ aliphatic)-. In some embodiments, L is —(C₁₋₁₀ aliphatic)-Cy-O—(C₁₋₁₀ aliphatic)-. In some embodiments, L is —(C₁₋₁₀ aliphatic)-Cy-(C₁₋₁₀ aliphatic)-O—. In some embodiments, L is —(C₁₋₁₀ aliphatic)-Cy-(C₁₋₁₀ aliphatic)-O—(C₁₋₁₀ aliphatic)-. In some embodiments, L is -Cy-(C₁₋₁₀ aliphatic)-Cy-O—. In some embodiments, L is -Cy-(C₁₋₁₀ aliphatic)-O-Cy-. In some embodiments, L is -Cy-(C₁₋₁₀ aliphatic)-Cy-O—(C₁₋₁₀ aliphatic)-. In some embodiments, L is -Cy-(C₁₋₁₀ aliphatic)-O-Cy-(C₁₋₁₀ aliphatic)-.

In some embodiments, L is -Cy-(C₁₋₁₀ aliphatic)-. In some embodiments, L is —(C₁₋₁₀ aliphatic)-Cy-(C₁₋₁₀ aliphatic)-. In some embodiments, L is —(C₁₋₁₀ aliphatic)-Cy-(CH₂CH₂O)₁₋₁₀CH₂CH₂—. In some embodiments, L is -Cy-(C₁₋₁₀ aliphatic)-Cy-. In some embodiments, L is -Cy-(C₁₋₁₀ aliphatic)-Cy-(C₁₋₁₀ aliphatic)-. In some embodiments, L is -Cy-(C₁₋₁₀ aliphatic)-Cy-(C₁₋₁₀ aliphatic)-Cy-. In some embodiments, L is —(C₁₋₁₀ aliphatic)-Cy-(C₁₋₁₀ aliphatic)-Cy-(C₁₋₁₀ aliphatic)-.

In some embodiments, L is —NR—(CH₂)₁₋₁₀—. In some embodiments, L is —(CH₂)₁₋₁₀—NR—(CH₂)₁₋₁₀—. In some embodiments, L is —(CH₂)₁₋₁₀—NR—(CH₂CH₂O)₁₋₁₀CH₂CH₂—. In some embodiments, L is -Cy-NR—(CH₂)₁₋₁₀—. In some embodiments, L is -Cy-(CH₂)₁₋₁₀—NR—. In some embodiments, L is -Cy-(CH₂)₁₋₁₀—NR—(CH₂)₁₋₁₀—. In some embodiments, L is —(CH₂)₁₋₁₀-Cy-NR—(CH₂)₁₋₁₀—. In some embodiments, L is —(CH₂)₁₋₁₀-Cy-(CH₂)₁₋₁₀—NR—. In some embodiments, L is —(CH₂)₁₋₁₀-Cy-(CH₂)₁₋₁₀—NR—(CH₂)₁₋₁₀—. In some embodiments, L is -Cy-(CH₂)₁₋₁₀-Cy-NR—. In some embodiments, L is -Cy-(CH₂)₁₋₁₀—NR-Cy-. In some embodiments, L is -Cy-(CH₂)₁₋₁₀-Cy-NR—(CH₂)₁₋₁₀—. In some embodiments, L is -Cy-(CH₂)₁₋₁₀—NR-Cy-(CH₂)₁₋₁₀—.

In some embodiments, L is —CONR—(CH₂)₁₋₁₀—. In some embodiments, L is —(CH₂)₁₋₁₀—CONR—(CH₂)₁₋₁₀—. In some embodiments, L is —(CH₂)₁₋₁₀—CONR—(CH₂CH₂O)₁₋₁₀CH₂CH₂—. In some embodiments, L is -Cy-CONR—(CH₂)₁₋₁₀—. In some embodiments, L is -Cy-(CH₂)₁₋₁₀—CONR—. In some embodiments, L is -Cy-(CH₂)₁₋₁₀—CONR—(CH₂)₁₋₁₀—. In some embodiments, L is —(CH₂)₁₋₁₀-Cy-CONR—(CH₂)₁₋₁₀—. In some embodiments, L is —(CH₂)₁₋₁₀-Cy-(CH₂)₁₋₁₀—CONR—. In some embodiments, L is —(CH₂)₁₋₁₀-Cy-(CH₂)₁₋₁₀—CONR—(CH₂)₁₋₁₀—. In some embodiments, L is -Cy-(CH₂)₁₋₁₀-Cy-CONR—. In some embodiments, L is -Cy-(CH₂)₁₋₁₀—CONR-Cy-. In some embodiments, L is -Cy-(CH₂)₁₋₁₀-Cy-CONR—(CH₂)₁₋₁₀—. In some embodiments, L is -Cy-(CH₂)₁₋₁₀—CONR-Cy-(CH₂)₁₋₁₀—.

In some embodiments, L is —NRCO—(CH₂)₁₋₁₀—. In some embodiments, L is —(CH₂)₁₋₁₀—NRCO—(CH₂)₁₋₁₀—. In some embodiments, L is —(CH₂)₁₋₁₀—NRCO—(CH₂CH₂O)₁₋₁₀CH₂CH₂—. In some embodiments, L is -Cy-NRCO—(CH₂)₁₋₁₀—. In some embodiments, L is -Cy-(CH₂)₁₋₁₀—NRCO—. In some embodiments, L is -Cy-(CH₂)₁₋₁₀—NRCO—(CH₂)₁₋₁₀—. In some embodiments, L is —(CH₂)₁₋₁₀-Cy-NRCO—(CH₂)₁₋₁₀—. In some embodiments, L is —(CH₂)₁₋₁₀-Cy-(CH₂)₁₋₁₀—NRCO—. In some embodiments, L is —(CH₂)₁₋₁₀-Cy-(CH₂)₁₋₁₀—NRCO—(CH₂)₁₋₁₀—. In some embodiments, L is -Cy-(CH₂)₁₋₁₀-Cy-NRCO—. In some embodiments, L is -Cy-(CH₂)₁₋₁₀—NRCO-Cy-. In some embodiments, L is -Cy-(CH₂)₁₋₁₀-Cy-NRCO—(CH₂)₁₋₁₀—. In some embodiments, L is -Cy-(CH₂)₁₋₁₀—NRCO-Cy-(CH₂)₁₋₁₀—.

In some embodiments, L is —O—(CH₂)₁₋₁₀—. In some embodiments, L is —(CH₂)₁₋₁₀—O—(CH₂)₁₋₁₀—. In some embodiments, L is —(CH₂)₁₋₁₀—O—(CH₂CH₂O)₁₋₁₀CH₂CH₂—. In some embodiments, L is -Cy-O—(CH₂)₁₋₁₀—. In some embodiments, L is -Cy-(CH₂)₁₋₁₀—O—. In some embodiments, L is -Cy-(CH₂)₁₋₁₀—(CH₂)₁₋₁₀—. In some embodiments, L is —(CH₂)₁₋₁₀-Cy-O—(CH₂)₁₋₁₀—. In some embodiments, L is —(CH₂)₁₋₁₀-Cy-(CH₂)₁₋₁₀—O—. In some embodiments, L is —(CH₂)₁₋₁₀-Cy-(CH₂)₁₋₁₀—O—(CH₂)₁₋₁₀—. In some embodiments, L is -Cy-(CH₂)₁₋₁₀-Cy-O—. In some embodiments, L is -Cy-(CH₂)₁₋₁₀—O-Cy-. In some embodiments, L is -Cy-(CH₂)₁₋₁₀-Cy-O—(CH₂)₁₋₁₀—. In some embodiments, L is -Cy-(CH₂)₁₋₁₀—O-Cy-(CH₂)₁₋₁₀—.

In some embodiments, L is -Cy-(CH₂)₁₋₁₀—. In some embodiments, L is —(CH₂)₁₋₁₀-Cy-(CH₂)₁₋₁₀—. In some embodiments, L is —(CH₂)₁₋₁₀-Cy-(CH₂CH₂O)₁₋₁₀CH₂CH₂—. In some embodiments, L is -Cy-(CH₂)₁₋₁₀-Cy-. In some embodiments, L is -Cy-(CH₂)₁₋₁₀-Cy-(CH₂)₁₋₁₀—. In some embodiments, L is -Cy-(CH₂)₁₋₁₀-Cy-(CH₂)₁₋₁₀-Cy-. In some embodiments, L is —(CH₂)₁₋₁₀-Cy-(CH₂)₁₋₁₀-Cy-(CH₂)₁₋₁₀—.

In some embodiments, L is —O-Cy-(CH₂)₁₋₁₀-Cy-. In some embodiments, L is -Cy-O-Cy-(CH₂)₁₋₁₀-Cy-. In some embodiments, L is —NR-Cy-O-Cy-(CH₂)₁₋₁₀-Cy-.

In some embodiments, L is a covalent bond. In some embodiments, L is

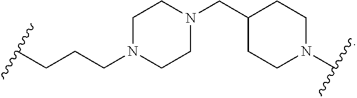 [SMILES: *CCCN1CCN(CC2CCN(*)CC2)CC1]

In some embodiments, L is

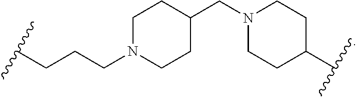 [SMILES: *CCCN1CCC(CN2CCC(*)CC2)CC1]

In some embodiments, L is

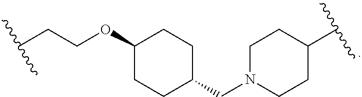 [SMILES: *CCO[C@H]1CC[C@H](CN2CCC(*)CC2)CC1]

In some embodiments, L is

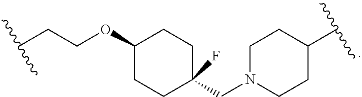 [SMILES: *CCO[C@H]1CC[C@](F)(CN2CCC(*)CC2)CC1]

In some embodiments, L is

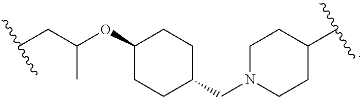 [SMILES: *CC(C)O[C@H]1CC[C@H](CN2CCC(*)CC2)CC1]

In some embodiments, L is

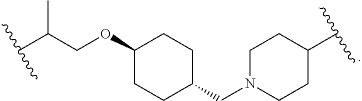 [SMILES: *C(C)CO[C@H]1CC[C@H](CN2CCC(*)CC2)CC1]

In some embodiments, L is

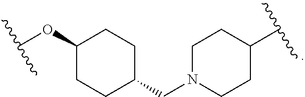 [SMILES: *O[C@H]1CC[C@H](CN2CCC(*)CC2)CC1]

In some embodiments, L is

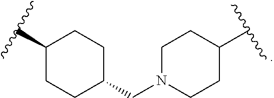 [SMILES: *C1CCN(C[C@H]2CC[C@H](*)CC2)CC1]

In some embodiments, L is

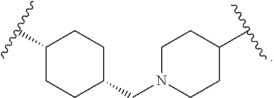 [SMILES: *C1CCN(C[C@H]2CC[C@@H](*)CC2)CC1]

In some embodiments, L is

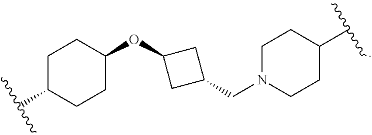 [SMILES: *C1CCN(C[C@H]2C[C@H](O[C@H]3CC[C@H](*)CC3)C2)CC1]

In some embodiments, L is

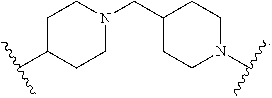 [SMILES: *C1CCN(CC2CCN(*)CC2)CC1]

In some embodiments, L is

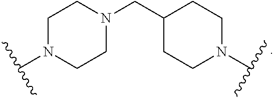 [SMILES: *N1CCC(CN2CCN(*)CC2)CC1]

In some embodiments, L is

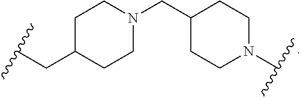 [SMILES: *CC1CCN(CC2CCN(*)CC2)CC1]

In some embodiments, L is

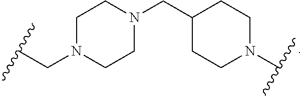 [SMILES: *CN1CCN(CC2CCN(*)CC2)CC1]

In some embodiments, L is

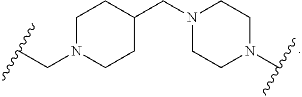 [SMILES: *CN1CCC(CN2CCN(*)CC2)CC1]

In some embodiments, L is

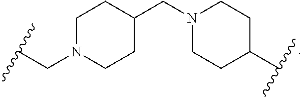 [SMILES: *CN1CCC(CN2CCC(*)CC2)CC1]

In some embodiments, L is

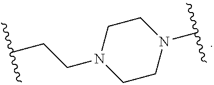 [SMILES: *CCN1CCN(*)CC1]

In some embodiments, L is

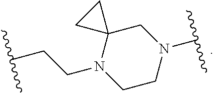 [SMILES: *CCN1CCN(*)CC12CC2]

In some embodiments, L is

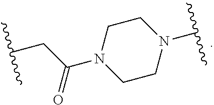 [SMILES: *CC(=O)N1CCN(*)CC1]

In some embodiments, L is

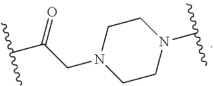 [SMILES: *C(=O)CN1CCN(*)CC1]

In some embodiments, L is

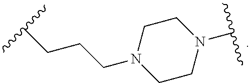 [SMILES: *CCCN1CCN(*)CC1]

In some embodiments, L is

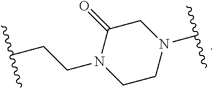 [SMILES: *CCN1CCN(*)CC1=O]

In some embodiments, L is

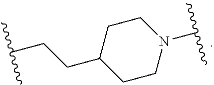 [SMILES: *CCC1CCN(*)CC1]

In some embodiments, L is

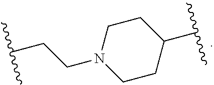 [SMILES: *CCN1CCC(*)CC1]

In some embodiments, L is

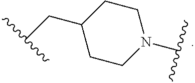 [SMILES: *CC1CCN(*)CC1]

In some embodiments, L is

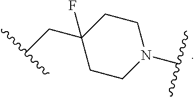 [SMILES: *CC1(F)CCN(*)CC1]

In some embodiments, L is

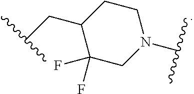 [SMILES: *CC1CCN(*)CC1(F)F]

In some embodiments, L is

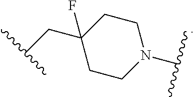 [SMILES: *CC1(F)CCN(*)CC1]

In some embodiments, L is

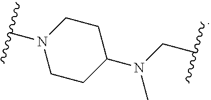 [SMILES: *CN(C)C1CCN(*)CC1]

In some embodiments, L is

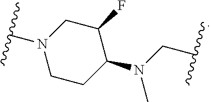 [SMILES: *CN(C)[C@H]1CCN(*)C[C@H]1F]

In some embodiments, L is

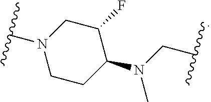 [SMILES: *CN(C)[C@H]1CCN(*)C[C@@H]1F]

In some embodiments, L is

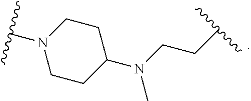 [SMILES: *CCN(C)C1CCN(*)CC1]

In some embodiments, L is

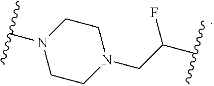 [SMILES: *C(F)CN1CCN(*)CC1]

In some embodiments, L is

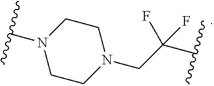 [SMILES: *N1CCN(CC(*)(F)F)CC1]

In some embodiments, L is

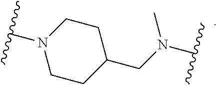 [SMILES: *N(C)CC1CCN(*)CC1]

In some embodiments, L is

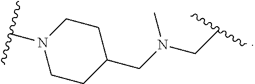 [SMILES: *CN(C)CC1CCN(*)CC1]

In some embodiments, L is

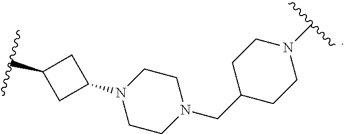 [SMILES: *N1CCC(CN2CCN([C@H]3C[C@H](*)C3)CC2)CC1]

In some embodiments, L is

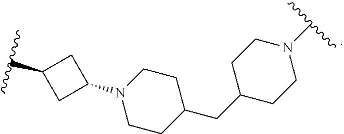 [SMILES: *N1CCC(CC2CCN([C@H]3C[C@H](*)C3)CC2)CC1]

In some embodiments, L is

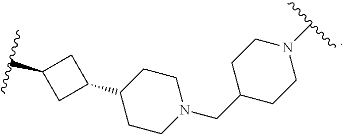 [SMILES: *N1CCC(CN2CCC([C@H]3C[C@H](*)C3)CC2)CC1]

In some embodiments, L is

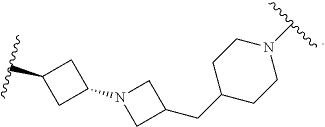 [SMILES: *N1CCC(CC2CN([C@H]3C[C@H](*)C3)C2)CC1]

In some embodiments, L is

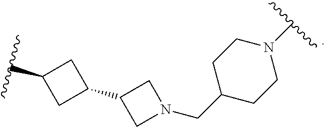 [SMILES: *N1CCC(CN2CC([C@H]3C[C@H](*)C3)C2)CC1]

In some embodiments, L is

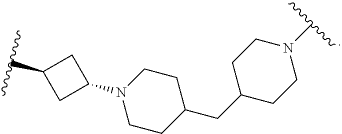 [SMILES: *N1CCC(CC2CCN([C@H]3C[C@H](*)C3)CC2)CC1]

In some embodiments, L is

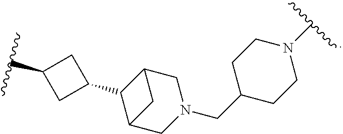 [SMILES: *N1CCC(CN2CC3CC(C2)C3[C@H]2C[C@H](*)C2)CC1]

In some embodiments, L is

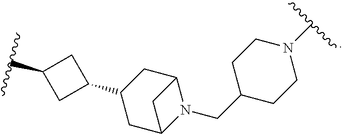 [SMILES: *N1CCC(CN2C3CC2CC([C@H]2C[C@H](*)C2)C3)CC1]

In some embodiments, L is

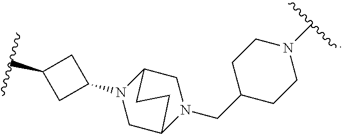 [SMILES: *N1CCC(CN2CC3CCC2CN3[C@H]2C[C@H](*)C2)CC1]

In some embodiments, L is

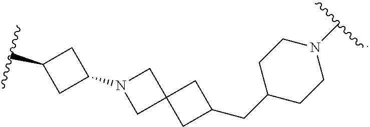 [SMILES: *N1CCC(CC2CC3(C2)CN([C@H]2C[C@H](*)C2)C3)CC1]

In some embodiments, L is

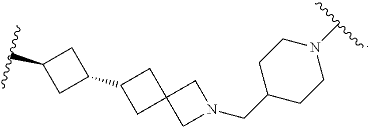 [SMILES: *N1CCC(CN2CC3(CC([C@H]4C[C@H](*)C4)C3)C2)CC1]

In some embodiments, L is

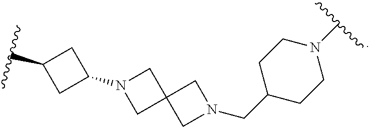 [SMILES: *N1CCC(CN2CC3(C2)CN([C@H]2C[C@H](*)C2)C3)CC1]

In some embodiments, L is

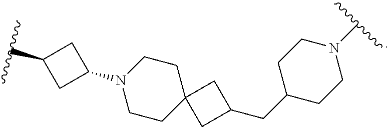 [SMILES: *N1CCC(CC2CC3(CCN([C@H]4C[C@H](*)C4)CC3)C2)CC1]

In some embodiments, L is

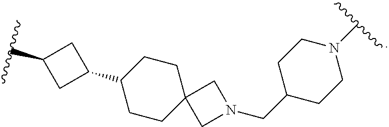 [SMILES: *N1CCC(CN2CC3(CCC([C@H]4C[C@H](*)C4)CC3)C2)CC1]

In some embodiments, L is

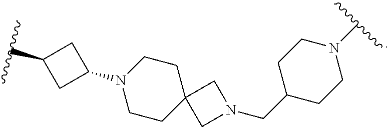 [SMILES: *N1CCC(CN2CC3(CCN([C@H]4C[C@H](*)C4)CC3)C2)CC1]

In some embodiments, L is

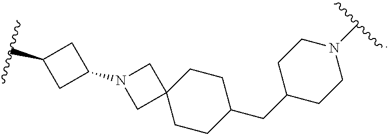 [SMILES: *N1CCC(CC2CCC3(CC2)CN([C@H]2C[C@H](*)C2)C3)CC1]

In some embodiments, L is

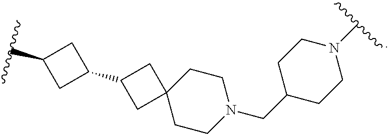 [SMILES: *N1CCC(CN2CCC3(CC2)CC([C@H]2C[C@H](*)C2)C3)CC1]

In some embodiments, L is

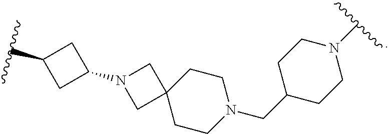 [SMILES: *N1CCC(CN2CCC3(CC2)CN([C@H]2C[C@H](*)C2)C3)CC1]

In some embodiments, L is

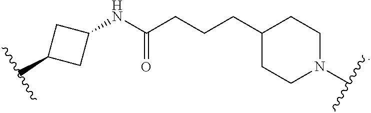 [SMILES: *N1CCC(CCCC(=O)N[C@H]2C[C@H](*)C2)CC1]

In some embodiments, L is

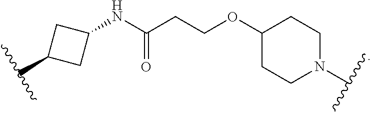 [SMILES: *N1CCC(OCCC(=O)N[C@H]2C[C@H](*)C2)CC1]

In some embodiments, L is

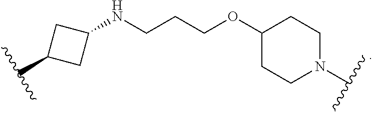 [SMILES: *N1CCC(OCCCN[C@H]2C[C@H](*)C2)CC1]

In some embodiments, L is

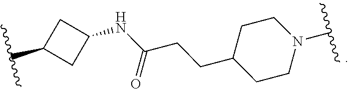 [SMILES: *N1CCC(CCC(=O)N[C@H]2C[C@H](*)C2)CC1]

In some embodiments, L is

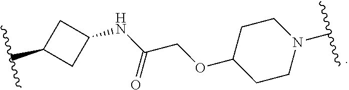 [SMILES: *N1CCC(OCC(=O)N[C@H]2C[C@H](*)C2)CC1]

In some embodiments, L is

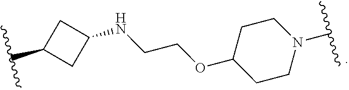 [SMILES: *N1CCC(OCCN[C@H]2C[C@H](*)C2)CC1]

In some embodiments, L is

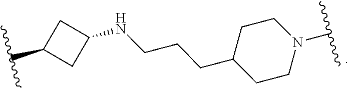 [SMILES: *N1CCC(CCCN[C@H]2C[C@H](*)C2)CC1]

In some embodiments, L is

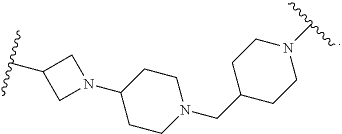 [SMILES: *C1CN(C2CCN(CC3CCN(*)CC3)CC2)C1]

In some embodiments, L is

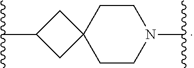 [SMILES: *C1CC2(CCN(*)CC2)C1]

In some embodiments, L is

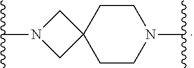 [SMILES: *N1CCC2(CC1)CN(*)C2]

In some embodiments, L is

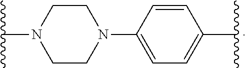 [SMILES: *c1ccc(N2CCN(*)CC2)cc1]

In some embodiments, L is

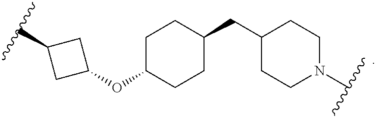 [SMILES: *N1CCC(C[C@H]2CC[C@H](O[C@H]3C[C@H](*)C3)CC2)CC1]

In some embodiments, L is

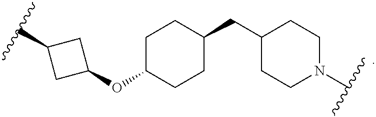 [SMILES: *N1CCC(C[C@H]2CC[C@H](O[C@H]3C[C@@H](*)C3)CC2)CC1]

In some embodiments, L is

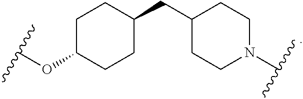 [SMILES: *O[C@H]1CC[C@H](CC2CCN(*)CC2)CC1]

In some embodiments, L is

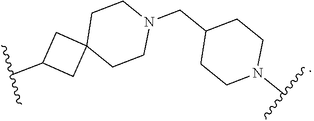 [SMILES: *C1CC2(CCN(CC3CCN(*)CC3)CC2)C1]

In some embodiments, L is

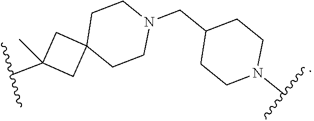 [SMILES: *N1CCC(CN2CCC3(CC2)CC(*)(C)C3)CC1]

In some embodiments, L is

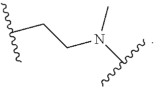 [SMILES: *CCN(*)C]

In some embodiments, L is

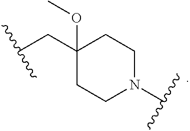 [SMILES: *CC1(OC)CCN(*)CC1]

In some embodiments, L is

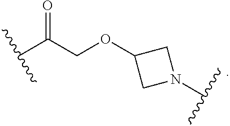 [SMILES: *C(=O)COC1CN(*)C1]

In some embodiments, L is

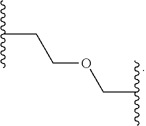 [SMILES: *CCOC*]

In some embodiments, L is

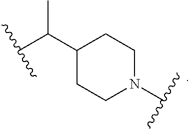 [SMILES: *C(C)C1CCN(*)CC1]

In some embodiments, L is

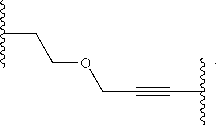 [SMILES: *C#CCOCC*]

In some embodiments, L is

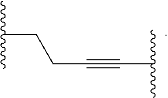 [SMILES: *C#CCC*]

In some embodiments, L is

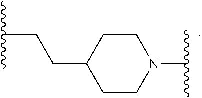 [SMILES: *CCC1CCN(*)CC1]

In some embodiments, L is

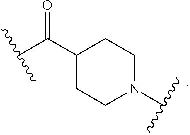 [SMILES: *C(=O)C1CCN(*)CC1]

In some embodiments, L is

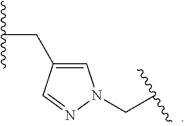 [SMILES: *Cc1cnn(C*)c1]

In some embodiments, L is

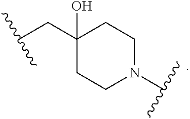 [SMILES: *CC1(O)CCN(*)CC1]

In some embodiments, L is

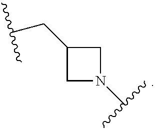 [SMILES: *CC1CN(*)C1]

In some embodiments, L is

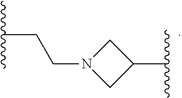 [SMILES: *CCN1CC(*)C1]

In some embodiments, L is

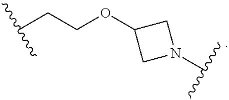 [SMILES: *CCOC1CN(*)C1]

In some embodiments, L is

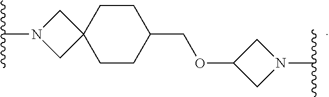 [SMILES: *N1CC(OCC2CCC3(CC2)CN(*)C3)C1]

In some embodiments, L is

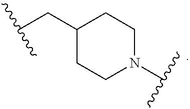 [SMILES: *CC1CCN(*)CC1]

In some embodiments, L is

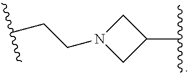 [SMILES: *CCN1CC(*)C1]

In some embodiments, L is

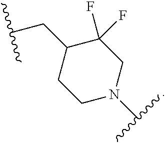 [SMILES: *CC1CCN(*)CC1(F)F]

In some embodiments, L is

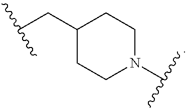 [SMILES: *CC1CCN(*)CC1]

In some embodiments, L is

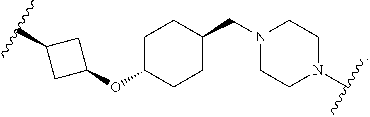 [SMILES: *N1CCN(C[C@H]2CC[C@H](O[C@H]3C[C@@H](*)C3)CC2)CC1]

In some embodiments, L is

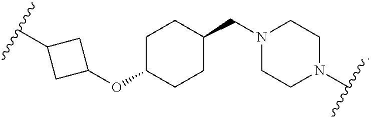 [SMILES: *C1CC(O[C@H]2CC[C@H](CN3CCN(*)CC3)CC2)C1]

In some embodiments, L is

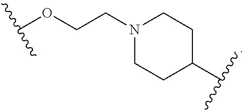 [SMILES: *OCCN1CCC(*)CC1]

In some embodiments, L is

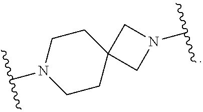 [SMILES: *N1CCC2(CC1)CN(*)C2]

In some embodiments, L is

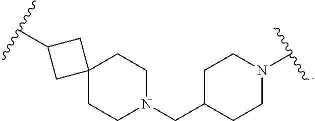 [SMILES: *C1CC2(CCN(CC3CCN(*)CC3)CC2)C1]

In some embodiments, L is

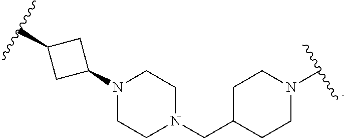 [SMILES: *N1CCC(CN2CCN([C@H]3C[C@@H](*)C3)CC2)CC1]

In some embodiments, L is

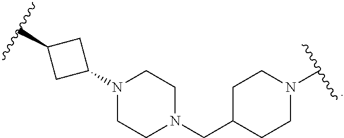 [SMILES: *N1CCC(CN2CCN([C@H]3C[C@H](*)C3)CC2)CC1]

In some embodiments, L is

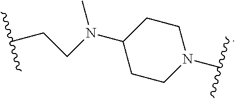 [SMILES: *CCN(C)C1CCN(*)CC1]

In some embodiments, L is

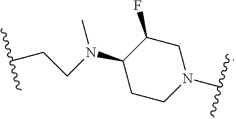 [SMILES: *CCN(C)[C@@H]1CCN(*)C[C@@H]1F]

In some embodiments, L is

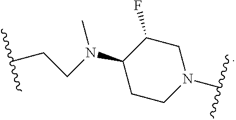 [SMILES: *CCN(C)[C@@H]1CCN(*)C[C@H]1F]

In some embodiments, L is

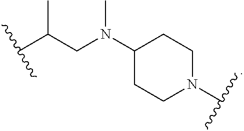 [SMILES: *C(C)CN(C)C1CCN(*)CC1]

In some embodiments, L is

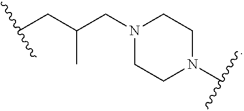 [SMILES: *CC(C)CN1CCN(*)CC1]

In some embodiments, L is

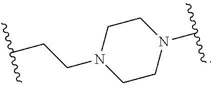 [SMILES: *CCN1CCN(*)CC1]

In some embodiments, L is

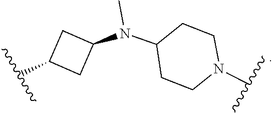 [SMILES: *N1CCC(N(C)[C@H]2C[C@H](*)C2)CC1]

In some embodiments, L is

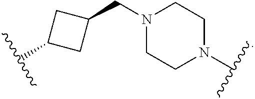 [SMILES: *N1CCN(C[C@H]2C[C@H](*)C2)CC1]

In some embodiments, L is

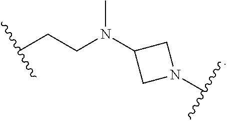 [SMILES: *CCN(C)C1CN(*)C1]

In some embodiments, L is

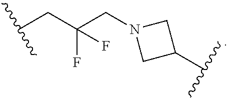 [SMILES: *CC(F)(F)CN1CC(*)C1]

In some embodiments, L is

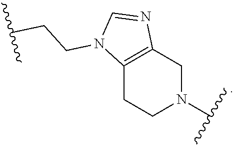 [SMILES: *CCn1cnc2c1CCN(*)C2]

In some embodiments, L is

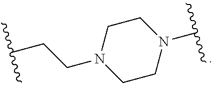 [SMILES: *CCN1CCN(*)CC1]

In some embodiments, L is

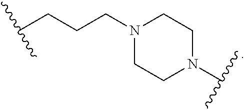 [SMILES: *CCCN1CCN(*)CC1]

In some embodiments, L is

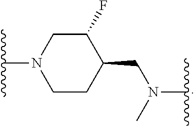 [SMILES: *N(C)C[C@H]1CCN(*)C[C@@H]1F]

In some embodiments, L is

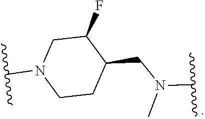 [SMILES: *N(C)C[C@H]1CCN(*)C[C@H]1F]

In some embodiments, L is

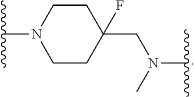 [SMILES: *N(C)CC1(F)CCN(*)CC1]

In some embodiments, L is

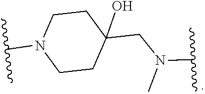 [SMILES: *N(C)CC1(O)CCN(*)CC1]

In some embodiments, L is

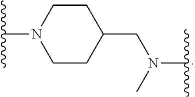 [SMILES: *N(C)CC1CCN(*)CC1]

In some embodiments, L is

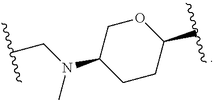 [SMILES: *CN(C)[C@@H]1CC[C@H](*)OC1]

In some embodiments, L is

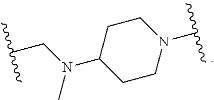 [SMILES: *CN(C)C1CCN(*)CC1]

In some embodiments, L is

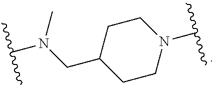 [SMILES: *N(C)CC1CCN(*)CC1]

In some embodiments, L is

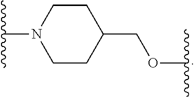 [SMILES: *OCC1CCN(*)CC1]

In some embodiments, L is

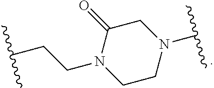 [SMILES: *CCN1CCN(*)CC1=O]

In some embodiments, L is

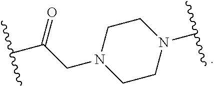 [SMILES: *C(=O)CN1CCN(*)CC1]

In some embodiments, L is

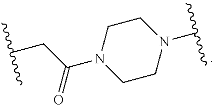 [SMILES: *CC(=O)N1CCN(*)CC1]

In some embodiments, L is

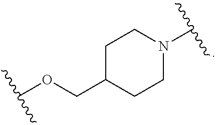 [SMILES: *OCC1CCN(*)CC1]

In some embodiments, L is

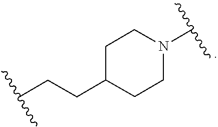 [SMILES: *CCC1CCN(*)CC1]

In some embodiments, L is

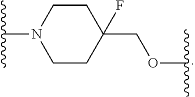 [SMILES: *OCC1(F)CCN(*)CC1]

In some embodiments, L is

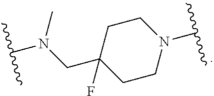 [SMILES: *N(C)CC1(F)CCN(*)CC1]

In some embodiments, L is

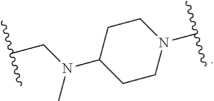 [SMILES: *CN(C)C1CCN(*)CC1]

In some embodiments, L is

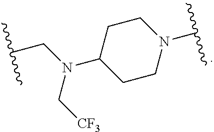 [SMILES: *CN(CC(F)(F)F)C1CCN(*)CC1]

In some embodiments, L is

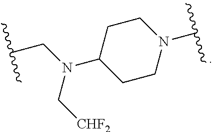 [SMILES: *CN(CC(F)F)C1CCN(*)CC1]

In some embodiments, L is

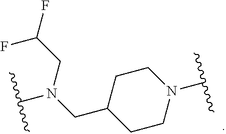 [SMILES: *N1CCC(CN(*)CC(F)F)CC1]

In some embodiments, L is

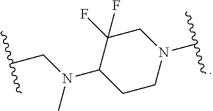 [SMILES: *CN(C)C1CCN(*)CC1(F)F]

In some embodiments, L is

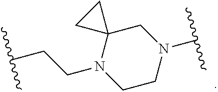 [SMILES: *CCN1CCN(*)CC12CC2]

In some embodiments, L is

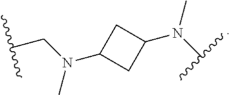 [SMILES: *CN(C)C1CC(N(*)C)C1]

In some embodiments, L is

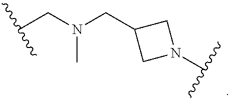 [SMILES: *CN(C)CC1CN(*)C1]

In some embodiments, L is

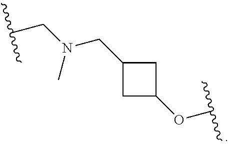 [SMILES: *CN(C)CC1CC(O*)C1]

In some embodiments, L is

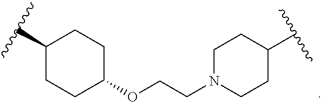 [SMILES: *C1CCN(CCO[C@H]2CC[C@H](*)CC2)CC1]

In some embodiments, L is

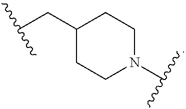 [SMILES: *CC1CCN(*)CC1]

In some embodiments, L is

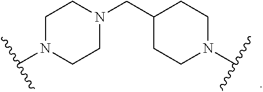 [SMILES: *N1CCC(CN2CCN(*)CC2)CC1]

In some embodiments, L is

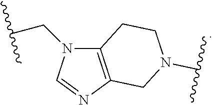 [SMILES: *Cn1cnc2c1CCN(*)C2]

In some embodiments, L is

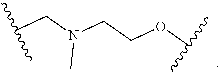 [SMILES: *CN(C)CCO*]

In some embodiments, L is

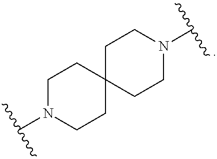 [SMILES: *N1CCC2(CC1)CCN(*)CC2]

In some embodiments, L is

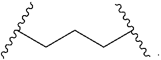 [SMILES: *CCC*]

In some embodiments, L is

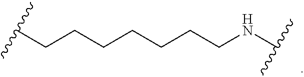 [SMILES: *CCCCCCCN*]

In some embodiments, L is

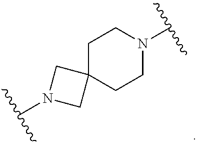 [SMILES: *N1CCC2(CC1)CN(*)C2]

In some embodiments, L is

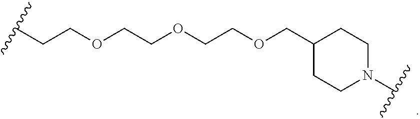 [SMILES: *CCOCCOCCOCC1CCN(*)CC1]

In some embodiments, L is v. In some embodiments, L is

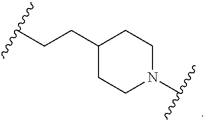 [SMILES: *CCC1CCN(*)CC1]

In some embodiments, L is

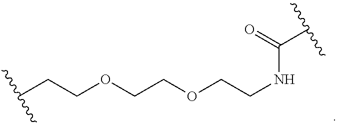 [SMILES: *CCOCCOCCNC(*)=O]

In some embodiments, L is

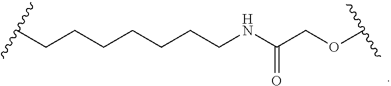 [SMILES: *CCCCCCCNC(=O)CO*]

In some embodiments, L is

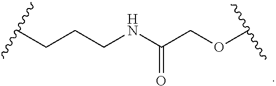 [SMILES: *CCCNC(=O)CO*]

In some embodiments, L is v. In some embodiments, L is

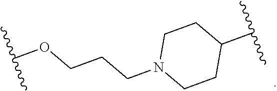 [SMILES: *OCCCN1CCC(*)CC1]

In some embodiments, L is

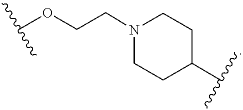 [SMILES: *OCCN1CCC(*)CC1]

In some embodiments, L is

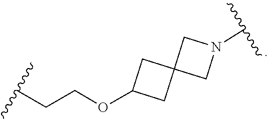 [SMILES: *CCOC1CC2(C1)CN(*)C2]

In some embodiments, L is

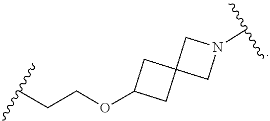 [SMILES: *CCOC1CC2(C1)CN(*)C2]

In some embodiments, L is

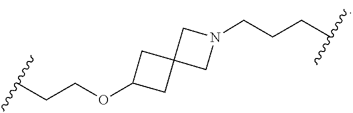 [SMILES: *CCCN1CC2(CC(OCC*)C2)C1]

In some embodiments, L is

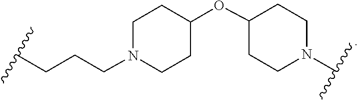 [SMILES: *CCCN1CCC(OC2CCN(*)CC2)CC1]

In some embodiments, L is

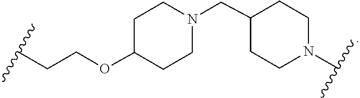 [SMILES: *CCOC1CCN(CC2CCN(*)CC2)CC1]

In some embodiments, L is

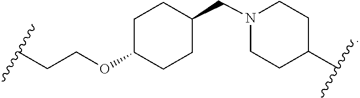 [SMILES: *CCO[C@H]1CC[C@H](CN2CCC(*)CC2)CC1]

In some embodiments, L is

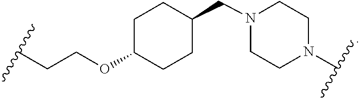 [SMILES: *CCO[C@H]1CC[C@H](CN2CCN(*)CC2)CC1]

In some embodiments, L is

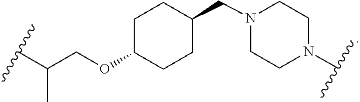 [SMILES: *C(C)CO[C@H]1CC[C@H](CN2CCN(*)CC2)CC1]

In some embodiments, L is

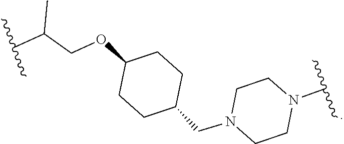 [SMILES: *C(C)CO[C@H]1CC[C@H](CN2CCN(*)CC2)CC1]

In some embodiments, L is

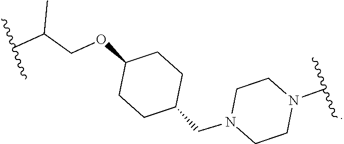 [SMILES: *C(C)CO[C@H]1CC[C@H](CN2CCN(*)CC2)CC1]

In some embodiments, L is

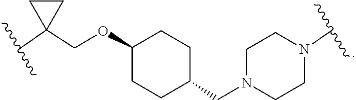 [SMILES: *N1CCN(C[C@H]2CC[C@H](OCC3(*)CC3)CC2)CC1]

In some embodiments, L is

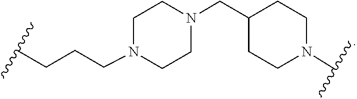 [SMILES: *CCCN1CCN(CC2CCN(*)CC2)CC1]

In some embodiments, L is

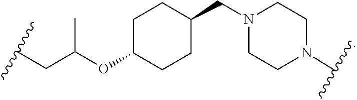 [SMILES: *CC(C)O[C@H]1CC[C@H](CN2CCN(*)CC2)CC1]

In some embodiments, L is

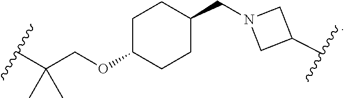 [SMILES: *C1CN(C[C@H]2CC[C@H](OCC(*)(C)C)CC2)C1]

In some embodiments, L is

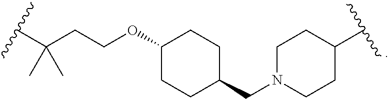 [SMILES: *C1CCN(C[C@H]2CC[C@H](OCCC(*)(C)C)CC2)CC1]

In some embodiments, L is

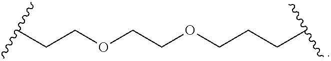 [SMILES: *CCCOCCOCC*]

In some embodiments, L is

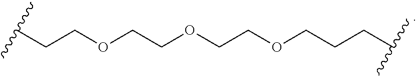 [SMILES: *CCCOCCOCCOCC*]

In some embodiments, L is

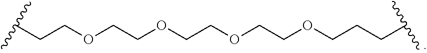 [SMILES: *CCCOCCOCCOCCOCC*]

In some embodiments, L is

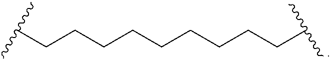 [SMILES: *CCCCCCCCC*]

In some embodiments, L is

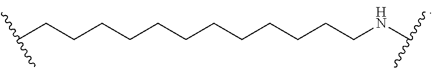 [SMILES: *CCCCCCCCCCCCN*]

In some embodiments, L is

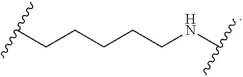 [SMILES: *CCCCCN*]

In some embodiments, L is

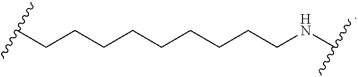 [SMILES: *CCCCCCCCCN*]

In some embodiments, L is

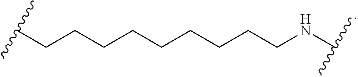 [SMILES: *CCCCCCCCCN*]

In some embodiments, L is

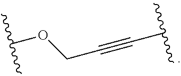 [SMILES: *C#CCO*]

In some embodiments, L is

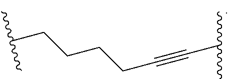 [SMILES: *C#CCCCC*]

In some embodiments, L is

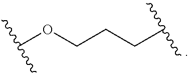 [SMILES: *CCCO*]

In some embodiments, L is

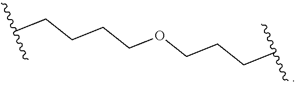 [SMILES: *CCCCOCCC*]

In some embodiments, L is

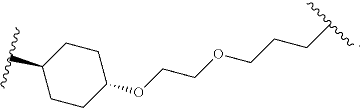 [SMILES: *CCCOCCO[C@H]1CC[C@H](*)CC1]

In some embodiments, L is

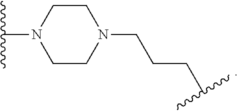 [SMILES: *CCCN1CCN(*)CC1]

In some embodiments, L is

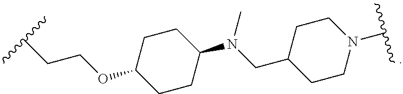 [SMILES: *CCO[C@H]1CC[C@H](N(C)CC2CCN(*)CC2)CC1]

In some embodiments, L is

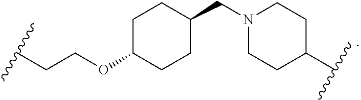 [SMILES: *CCO[C@H]1CC[C@H](CN2CCC(*)CC2)CC1]

In some embodiments, L is

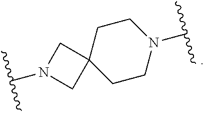 [SMILES: *N1CCC2(CC1)CN(*)C2]

In some embodiments, L is

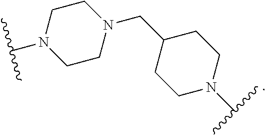 [SMILES: *N1CCC(CN2CCN(*)CC2)CC1]

In some embodiments, L is

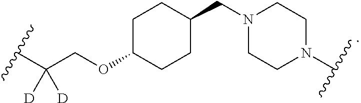 [SMILES: *N1CCN(C[C@H]2CC[C@H](OCC(*)([2H])[2H])CC2)CC1]

In some embodiments, L is D

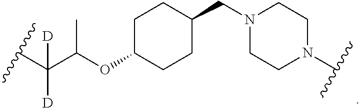 [SMILES: *N1CCN(C[C@H]2CC[C@H](OC(C)C(*)([2H])[2H])CC2)CC1]

In some embodiments, L is

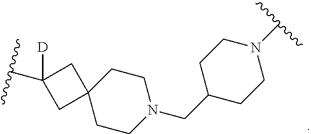 [SMILES: *N1CCC(CN2CCC3(CC2)CC(*)([2H])C3)CC1]

In some embodiments, L is

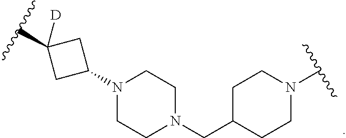 [SMILES: *N1CCC(CN2CCN([C@H]3C[C@@](*)([2H])C3)CC2)CC1]

In some embodiments, L is

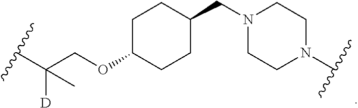 [SMILES: *N1CCN(C[C@H]2CC[C@H](OCC(*)([2H])C)CC2)CC1]

In some embodiments, L is

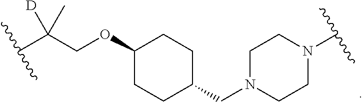 [SMILES: *N1CCN(C[C@H]2CC[C@H](OCC(*)([2H])C)CC2)CC1]

In some embodiments, L is

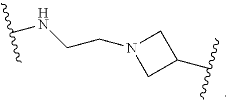 [SMILES: *NCCN1CC(*)C1]

In some embodiments, L is

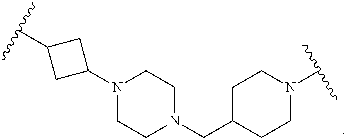 [SMILES: *C1CC(N2CCN(CC3CCN(*)CC3)CC2)C1]

In some embodiments, L is

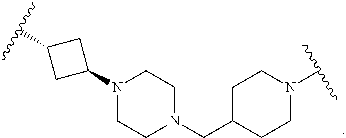 [SMILES: *N1CCC(CN2CCN([C@H]3C[C@H](*)C3)CC2)CC1]

In some embodiments, L is

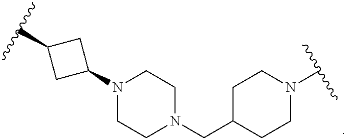 [SMILES: *N1CCC(CN2CCN([C@H]3C[C@@H](*)C3)CC2)CC1]

In some embodiments, L is

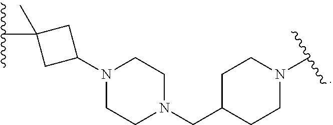 [SMILES: *N1CCC(CN2CCN(C3CC(*)(C)C3)CC2)CC1]

In some embodiments, L is

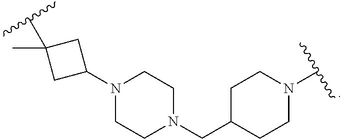 [SMILES: *N1CCC(CN2CCN(C3CC(*)(C)C3)CC2)CC1]

In some embodiments, L is

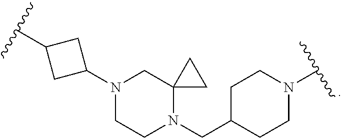 [SMILES: *C1CC(N2CCN(CC3CCN(*)CC3)C3(CC3)C2)C1]

In some embodiments, L is

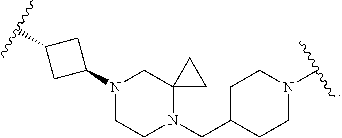 [SMILES: *N1CCC(CN2CCN([C@H]3C[C@H](*)C3)CC23CC3)CC1]

In some embodiments, L is

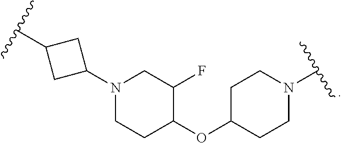 [SMILES: *C1CC(N2CCC(OC3CCN(*)CC3)C(F)C2)C1]

In some embodiments, L is

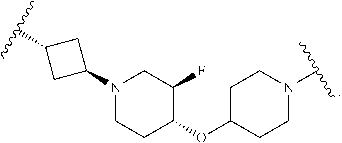 [SMILES: *N1CCC(O[C@@H]2CCN([C@H]3C[C@H](*)C3)C[C@H]2F)CC1]

In some embodiments, L is

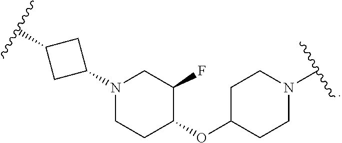 [SMILES: *N1CCC(O[C@@H]2CCN([C@H]3C[C@@H](*)C3)C[C@H]2F)CC1]

In some embodiments, L is

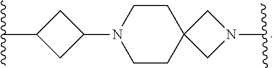 [SMILES: *C1CC(N2CCC3(CC2)CN(*)C3)C1]

In some embodiments, L is

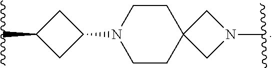 [SMILES: *N1CC2(CCN([C@H]3C[C@H](*)C3)CC2)C1]

In some embodiments, L is

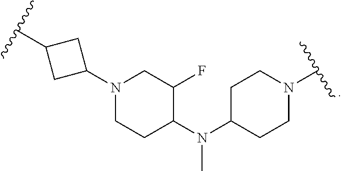 [SMILES: *C1CC(N2CCC(N(C)C3CCN(*)CC3)C(F)C2)C1]

In some embodiments, L is

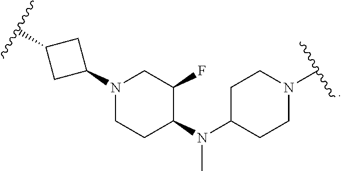 [SMILES: *N1CCC(N(C)[C@H]2CCN([C@H]3C[C@H](*)C3)C[C@H]2F)CC1]

In some embodiments, L is

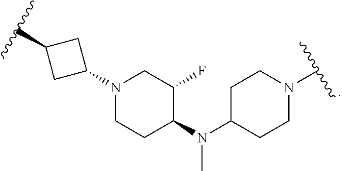 [SMILES: *N1CCC(N(C)[C@H]2CCN([C@H]3C[C@H](*)C3)C[C@@H]2F)CC1]

In some embodiments, L is

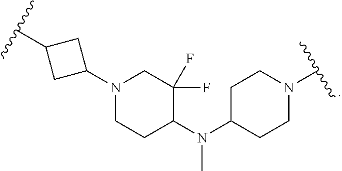 [SMILES: *C1CC(N2CCC(N(C)C3CCN(*)CC3)C(F)(F)C2)C1]

In some embodiments, L is

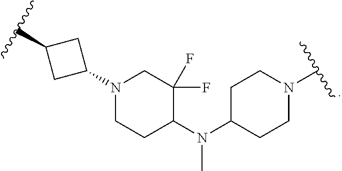 [SMILES: *N1CCC(N(C)C2CCN([C@H]3C[C@H](*)C3)CC2(F)F)CC1]

In some embodiments, L is

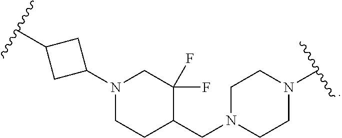 [SMILES: *C1CC(N2CCC(CN3CCN(*)CC3)C(F)(F)C2)C1]

In some embodiments, L is

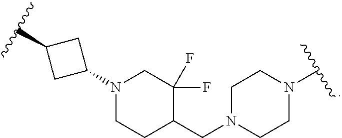 [SMILES: *N1CCN(CC2CCN([C@H]3C[C@H](*)C3)CC2(F)F)CC1]

In some embodiments, L is

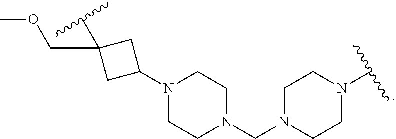 [SMILES: *N1CCN(CN2CCN(C3CC(*)(COC)C3)CC2)CC1]

In some embodiments, L is

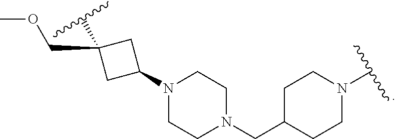 [SMILES: *N1CCC(CN2CCN([C@H]3C[C@@](*)(COC)C3)CC2)CC1]

In some embodiments, L is

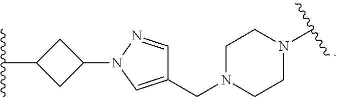 [SMILES: *C1CC(n2cc(CN3CCN(*)CC3)cn2)C1]

In some embodiments, L is

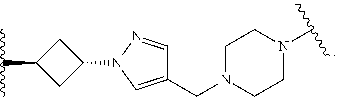 [SMILES: *N1CCN(Cc2cnn([C@H]3C[C@H](*)C3)c2)CC1]

In some embodiments, L is

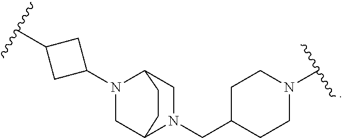 [SMILES: *C1CC(N2CC3CCC2CN3CC2CCN(*)CC2)C1]

In some embodiments, L is

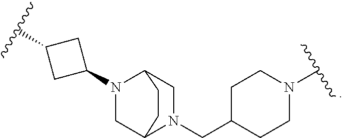 [SMILES: *N1CCC(CN2CC3CCC2CN3[C@H]2C[C@H](*)C2)CC1]

In some embodiments, L is

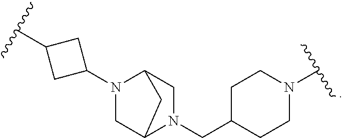 [SMILES: *C1CC(N2CC3CC2CN3CC2CCN(*)CC2)C1]

In some embodiments, L is

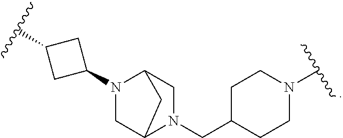 [SMILES: *N1CCC(CN2CC3CC2CN3[C@H]2C[C@H](*)C2)CC1]

In some embodiments, L is

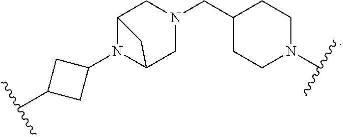 [SMILES: *C1CC(N2C3CC2CN(CC2CCN(*)CC2)C3)C1]

In some embodiments, L is

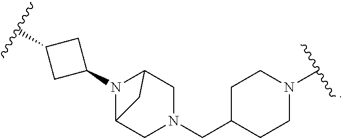 [SMILES: *N1CCC(CN2CC3CC(C2)N3[C@H]2C[C@H](*)C2)CC1]

In some embodiments, L is

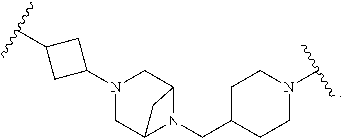 [SMILES: *C1CC(N2CC3CC(C2)N3CC2CCN(*)CC2)C1]

In some embodiments, L is

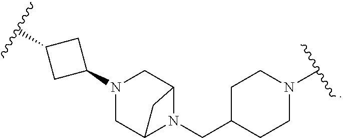 [SMILES: *N1CCC(CN2C3CC2CN([C@H]2C[C@H](*)C2)C3)CC1]

In some embodiments, L is

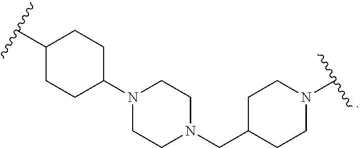 [SMILES: *C1CCC(N2CCN(CC3CCN(*)CC3)CC2)CC1]

In some embodiments, L is

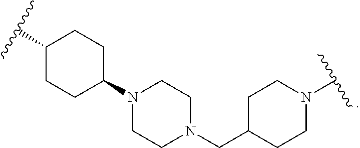 [SMILES: *N1CCC(CN2CCN([C@H]3CC[C@H](*)CC3)CC2)CC1]

In some embodiments, L is

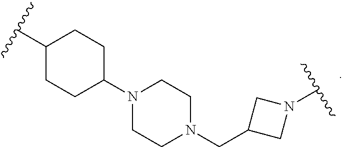 [SMILES: *C1CCC(N2CCN(CC3CN(*)C3)CC2)CC1]

In some embodiments, L is

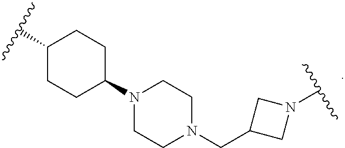 [SMILES: *N1CC(CN2CCN([C@H]3CC[C@H](*)CC3)CC2)C1]

In some embodiments, L is

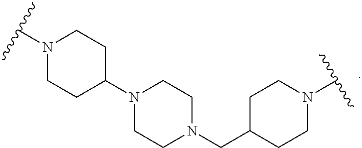 [SMILES: *N1CCC(CN2CCN(C3CCN(*)CC3)CC2)CC1]

In some embodiments, L is

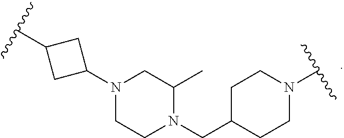 [SMILES: *C1CC(N2CCN(CC3CCN(*)CC3)C(C)C2)C1]

In some embodiments, L is

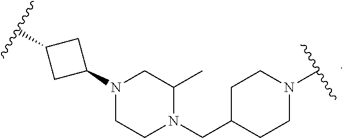 [SMILES: *N1CCC(CN2CCN([C@H]3C[C@H](*)C3)CC2C)CC1]

In some embodiments, L is

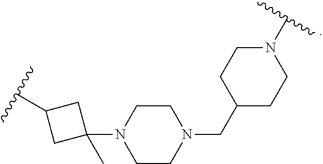 [SMILES: *C1CC(C)(N2CCN(CC3CCN(*)CC3)CC2)C1]

In some embodiments, L is

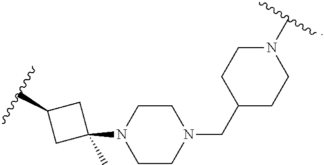 [SMILES: *N1CCC(CN2CCN([C@]3(C)C[C@H](*)C3)CC2)CC1]

In some embodiments, L is

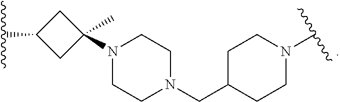 [SMILES: *N1CCC(CN2CCN([C@]3(C)C[C@@H](*)C3)CC2)CC1]

In some embodiments, L is

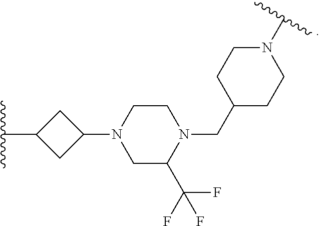 [SMILES: *C1CC(N2CCN(CC3CCN(*)CC3)C(C(F)(F)F)C2)C1]

In some embodiments, L is

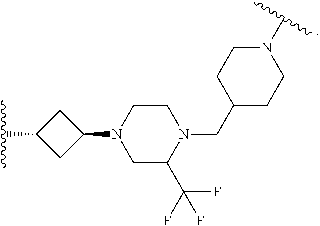 [SMILES: *N1CCC(CN2CCN([C@H]3C[C@H](*)C3)CC2C(F)(F)F)CC1]

In some embodiments, L is

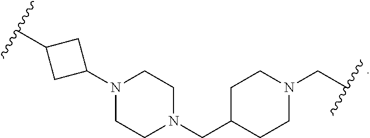 [SMILES: *CN1CCC(CN2CCN(C3CC(*)C3)CC2)CC1]

In some embodiments, L is

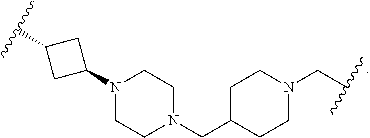 [SMILES: *CN1CCC(CN2CCN([C@H]3C[C@H](*)C3)CC2)CC1]

In some embodiments, L is

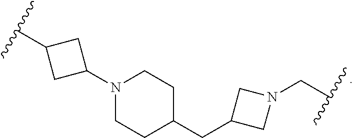 [SMILES: *CN1CC(CC2CCN(C3CC(*)C3)CC2)C1]

In some embodiments, L is

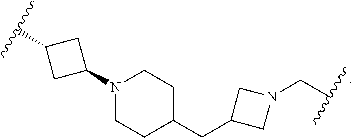 [SMILES: *CN1CC(CC2CCN([C@H]3C[C@H](*)C3)CC2)C1]

In some embodiments, L is

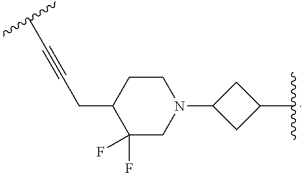 [SMILES: *C#CCC1CCN(C2CC(*)C2)CC1(F)F]

In some embodiments, L is

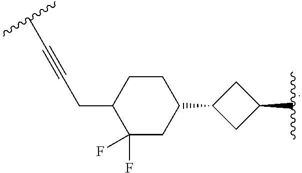 [SMILES: *C#CCC1CCC([C@H]2C[C@H](*)C2)CC1(F)F]

In some embodiments, L is

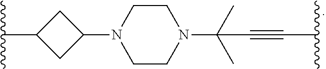 [SMILES: *C#CC(C)(C)N1CCN(C2CC(*)C2)CC1]

In some embodiments, L is

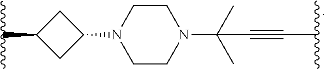 [SMILES: *C#CC(C)(C)N1CCN([C@H]2C[C@H](*)C2)CC1]

In some embodiments, L is

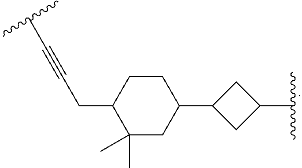 [SMILES: *C#CCC1CCC(C2CC(*)C2)CC1(C)C]

In some embodiments, L is

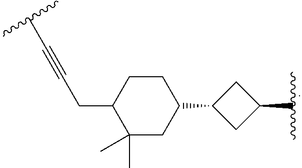 [SMILES: *C#CCC1CCC([C@H]2C[C@H](*)C2)CC1(C)C]

In some embodiments, L is

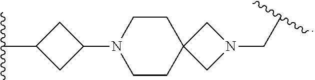 [SMILES: *CN1CC2(CCN(C3CC(*)C3)CC2)C1]

In some embodiments, L is

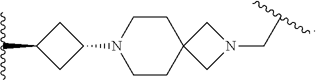 [SMILES: *CN1CC2(CCN([C@H]3C[C@H](*)C3)CC2)C1]

In some embodiments, L is

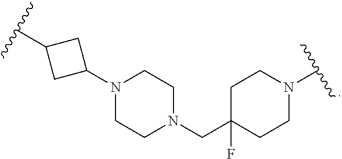 [SMILES: *C1CC(N2CCN(CC3(F)CCN(*)CC3)CC2)C1]

In some embodiments, L is

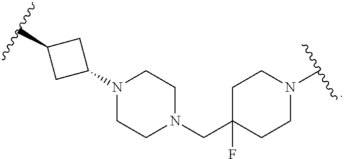 [SMILES: *N1CCC(F)(CN2CCN([C@H]3C[C@H](*)C3)CC2)CC1]

In some embodiments, L is

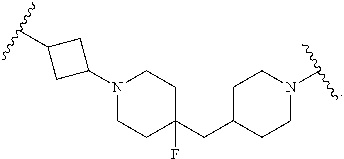 [SMILES: *C1CC(N2CCC(F)(CC3CCN(*)CC3)CC2)C1]

In some embodiments, L is

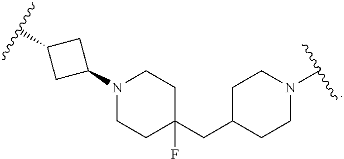 [SMILES: *N1CCC(CC2(F)CCN([C@H]3C[C@H](*)C3)CC2)CC1]

In some embodiments, L is

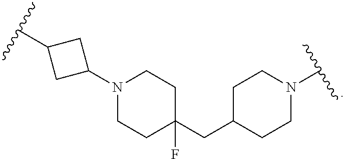 [SMILES: *C1CC(N2CCC(F)(CC3CCN(*)CC3)CC2)C1]

In some embodiments, L is

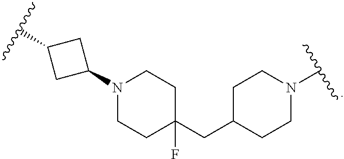 [SMILES: *N1CCC(CC2(F)CCN([C@H]3C[C@H](*)C3)CC2)CC1]

In some embodiments, L is

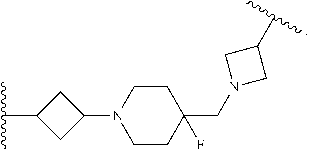 [SMILES: *C1CC(N2CCC(F)(CN3CC(*)C3)CC2)C1]

In some embodiments, L is

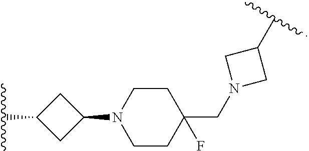 [SMILES: *C1CN(CC2(F)CCN([C@H]3C[C@H](*)C3)CC2)C1]

In some embodiments, L is

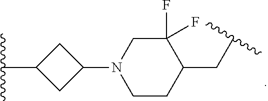 [SMILES: *CC1CCN(C2CC(*)C2)CC1(F)F]

In some embodiments, L is

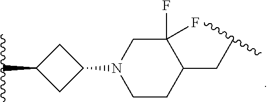 [SMILES: *CC1CCN([C@H]2C[C@H](*)C2)CC1(F)F]

In some embodiments, L is

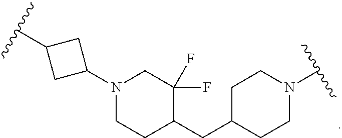 [SMILES: *C1CC(N2CCC(CC3CCN(*)CC3)C(F)(F)C2)C1]

In some embodiments, L is

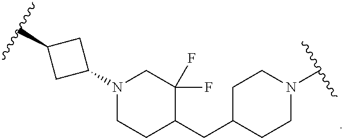 [SMILES: *N1CCC(CC2CCN([C@H]3C[C@H](*)C3)CC2(F)F)CC1]

In some embodiments, L is

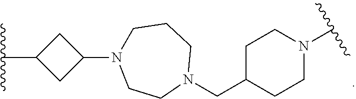 [SMILES: *C1CC(N2CCCN(CC3CCN(*)CC3)CC2)C1]

In some embodiments, L is

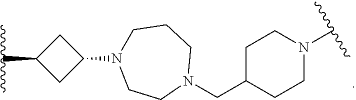 [SMILES: *N1CCC(CN2CCCN([C@H]3C[C@H](*)C3)CC2)CC1]

In some embodiments, L is

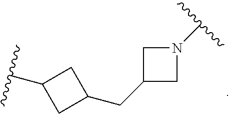 [SMILES: *C1CC(CC2CN(*)C2)C1]

In some embodiments, L is

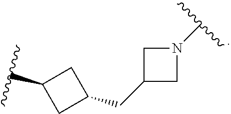 [SMILES: *N1CC(C[C@H]2C[C@H](*)C2)C1]

In some embodiments, L is

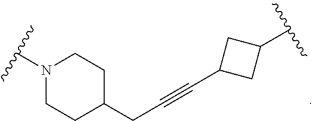 [SMILES: *C1CC(C#CCC2CCN(*)CC2)C1]

In some embodiments, L is

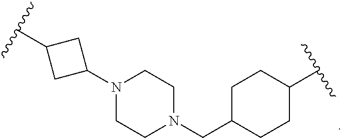 [SMILES: *C1CCC(CN2CCN(C3CC(*)C3)CC2)CC1]

In some embodiments, L is

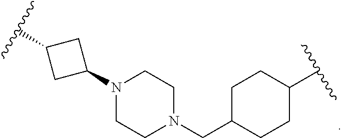 [SMILES: *C1CCC(CN2CCN([C@H]3C[C@H](*)C3)CC2)CC1]

In some embodiments, L is

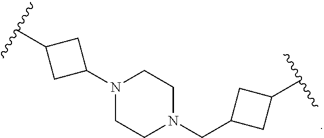 [SMILES: *C1CC(CN2CCN(C3CC(*)C3)CC2)C1]

In some embodiments, L is

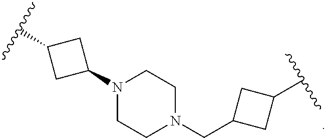 [SMILES: *C1CC(CN2CCN([C@H]3C[C@H](*)C3)CC2)C1]

In some embodiments, L is

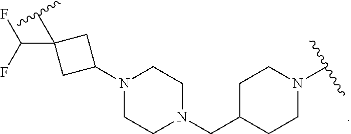 [SMILES: *N1CCC(CN2CCN(C3CC(*)(C(F)F)C3)CC2)CC1]

In some embodiments, L is

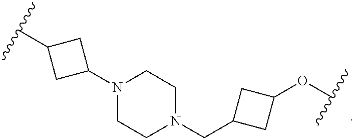 [SMILES: *OC1CC(CN2CCN(C3CC(*)C3)CC2)C1]

In some embodiments, L is

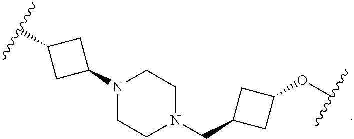 [SMILES: *O[C@H]1C[C@H](CN2CCN([C@H]3C[C@H](*)C3)CC2)C1]

In some embodiments, L is

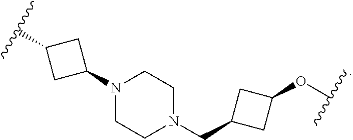 [SMILES: *O[C@H]1C[C@@H](CN2CCN([C@H]3C[C@H](*)C3)CC2)C1]

In some embodiments, L is

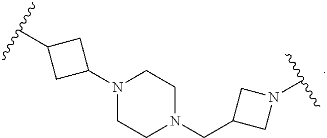 [SMILES: *C1CC(N2CCN(CC3CN(*)C3)CC2)C1]

In some embodiments, L is

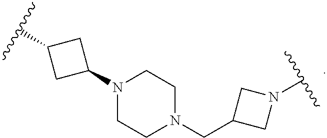 [SMILES: *N1CC(CN2CCN([C@H]3C[C@H](*)C3)CC2)C1]

In some embodiments, L is

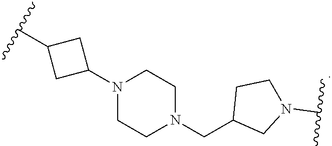 [SMILES: *C1CC(N2CCN(CC3CCN(*)C3)CC2)C1]

In some embodiments, L is

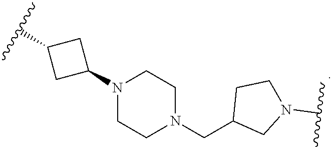 [SMILES: *N1CCC(CN2CCN([C@H]3C[C@H](*)C3)CC2)C1]

In some embodiments, L is

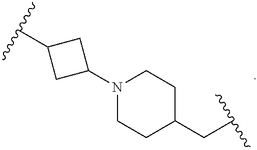 [SMILES: *CC1CCN(C2CC(*)C2)CC1]

In some embodiments, L is

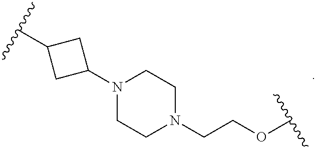 [SMILES: *OCCN1CCN(C2CC(*)C2)CC1]

In some embodiments, L is

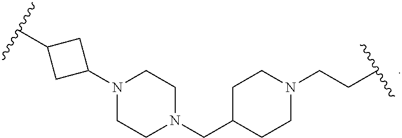 [SMILES: *CCN1CCC(CN2CCN(C3CC(*)C3)CC2)CC1]

In some embodiments, L is

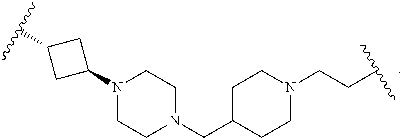 [SMILES: *CCN1CCC(CN2CCN([C@H]3C[C@H](*)C3)CC2)CC1]

In some embodiments, L is

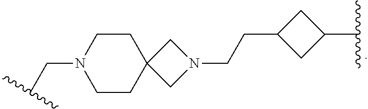 [SMILES: *CN1CCC2(CC1)CN(CCC1CC(*)C1)C2]

In some embodiments, L is

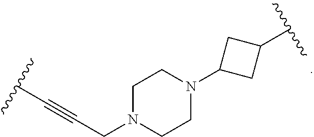 [SMILES: *C#CCN1CCN(C2CC(*)C2)CC1]

In some embodiments, L is

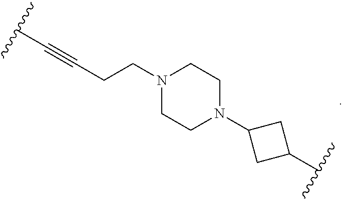 [SMILES: *C#CCCN1CCN(C2CC(*)C2)CC1]

In some embodiments, L is

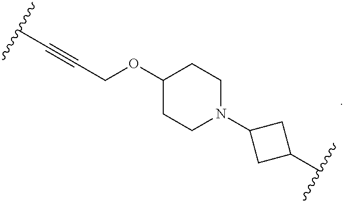 [SMILES: *C#CCOC1CCN(C2CC(*)C2)CC1]

In some embodiments, L is

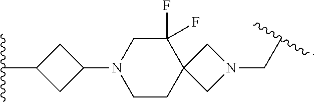 [SMILES: *CN1CC2(CCN(C3CC(*)C3)CC2(F)F)C1]

In some embodiments, L is

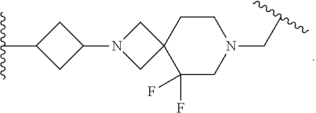 [SMILES: *CN1CCC2(CN(C3CC(*)C3)C2)C(F)(F)C1]

In some embodiments, L is

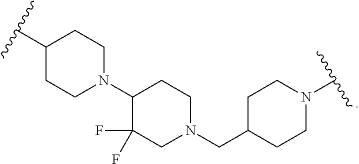 [SMILES: *C1CCN(C2CCN(CC3CCN(*)CC3)CC2(F)F)CC1]

In some embodiments, L is

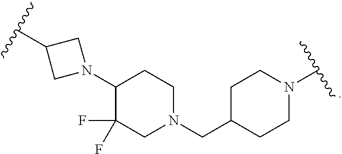 [SMILES: *C1CN(C2CCN(CC3CCN(*)CC3)CC2(F)F)C1]

In some embodiments, L is

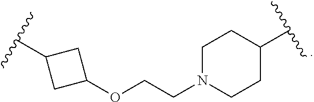 [SMILES: *C1CCN(CCOC2CC(*)C2)CC1]

In some embodiments, L is

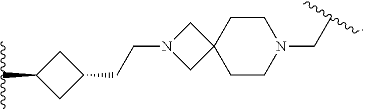 [SMILES: *CN1CCC2(CC1)CN(CC[C@H]1C[C@H](*)C1)C2]

In some embodiments, L is

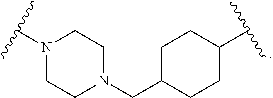 [SMILES: *C1CCC(CN2CCN(*)CC2)CC1]

In some embodiments, L is

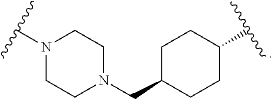 [SMILES: *N1CCN(C[C@H]2CC[C@H](*)CC2)CC1]

In some embodiments, L is

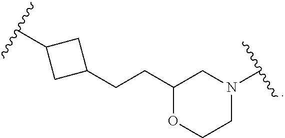 [SMILES: *C1CC(CCC2CN(*)CCO2)C1]

In some embodiments, L is

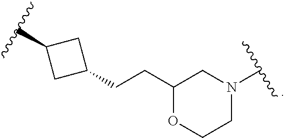 [SMILES: *N1CCOC(CC[C@H]2C[C@H](*)C2)C1]

In some embodiments, L is

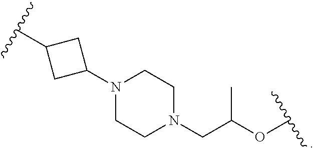 [SMILES: *OC(C)CN1CCN(C2CC(*)C2)CC1]

In some embodiments, L is

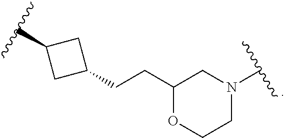 [SMILES: *N1CCOC(CC[C@H]2C[C@H](*)C2)C1]

In some embodiments, L is

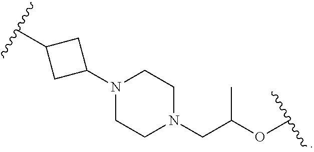 [SMILES: *OC(C)CN1CCN(C2CC(*)C2)CC1]

In some embodiments, L is

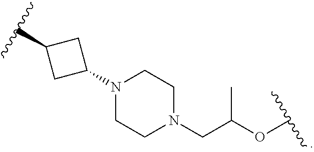 [SMILES: *OC(C)CN1CCN([C@H]2C[C@H](*)C2)CC1]

In some embodiments, L is

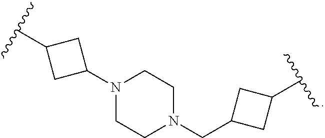 [SMILES: *C1CC(CN2CCN(C3CC(*)C3)CC2)C1]

In some embodiments, L is

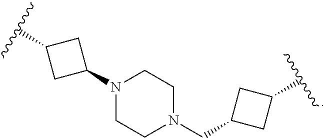 [SMILES: *[C@H]1C[C@@H](CN2CCN([C@H]3C[C@H](*)C3)CC2)C1]

In some embodiments, L is

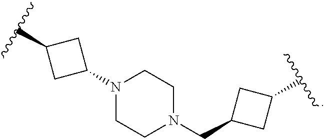 [SMILES: *[C@H]1C[C@H](CN2CCN([C@H]3C[C@H](*)C3)CC2)C1]

In some embodiments, L is

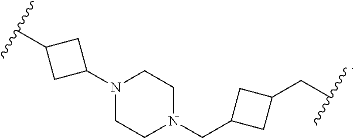 [SMILES: *CC1CC(CN2CCN(C3CC(*)C3)CC2)C1]

In some embodiments, L is

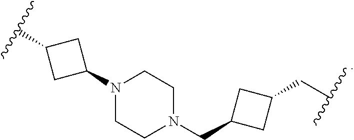 [SMILES: *C[C@H]1C[C@H](CN2CCN([C@H]3C[C@H](*)C3)CC2)C1]

In some embodiments, L is

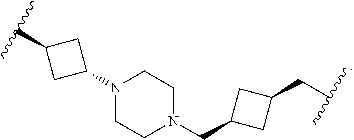 [SMILES: *C[C@H]1C[C@@H](CN2CCN([C@H]3C[C@H](*)C3)CC2)C1]

In some embodiments, L is

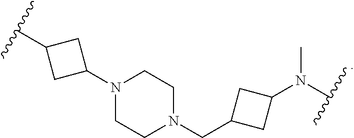 [SMILES: *C1CC(N2CCN(CC3CC(N(*)C)C3)CC2)C1]

In some embodiments, L is

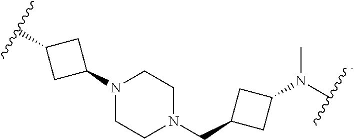 [SMILES: *N(C)[C@H]1C[C@H](CN2CCN([C@H]3C[C@H](*)C3)CC2)C1]

In some embodiments, L is

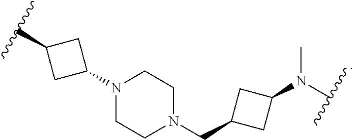 [SMILES: *N(C)[C@H]1C[C@@H](CN2CCN([C@H]3C[C@H](*)C3)CC2)C1]

In some embodiments, L is

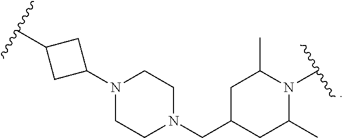 [SMILES: *C1CC(N2CCN(CC3CC(C)N(*)C(C)C3)CC2)C1]

In some embodiments, L is

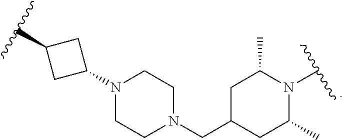 [SMILES: *N1[C@H](C)CC(CN2CCN([C@H]3C[C@H](*)C3)CC2)C[C@@H]1C]

In some embodiments, L is

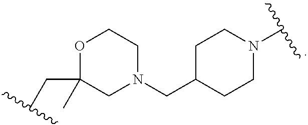 [SMILES: *CC1(C)CN(CC2CCN(*)CC2)CCO1]

In some embodiments, L is

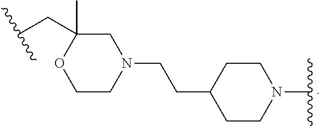 [SMILES: *CC1(C)CN(CCC2CCN(*)CC2)CCO1]

In some embodiments, L is

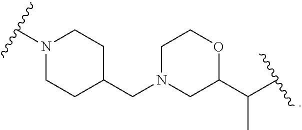 [SMILES: *C(C)C1CN(CC2CCN(*)CC2)CCO1]

In some embodiments, L is

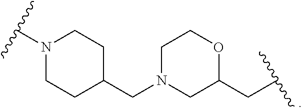 [SMILES: *CC1CN(CC2CCN(*)CC2)CCO1]

In some embodiments, L is

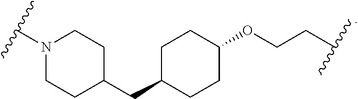 [SMILES: *CCO[C@H]1CC[C@H](CC2CCN(*)CC2)CC1]

In some embodiments, L is

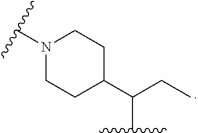 [SMILES: *C(CC)C1CCN(*)CC1]

In some embodiments, L is

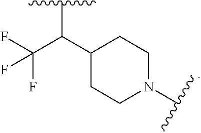 [SMILES: *C(C1CCN(*)CC1)C(F)(F)F]

In some embodiments, L is

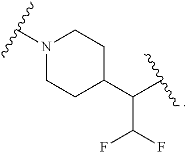 [SMILES: *C(C(F)F)C1CCN(*)CC1]

In some embodiments, L is

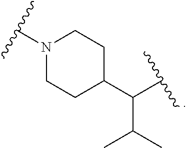 [SMILES: *C(C(C)C)C1CCN(*)CC1]

In some embodiments, L is

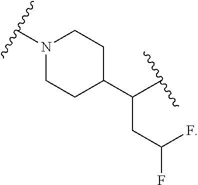 [SMILES: *C(CC(F)F)C1CCN(*)CC1]

In some embodiments, L is

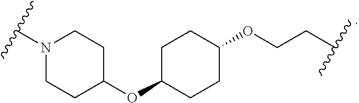 [SMILES: *CCO[C@H]1CC[C@H](OC2CCN(*)CC2)CC1]

In some embodiments, L is

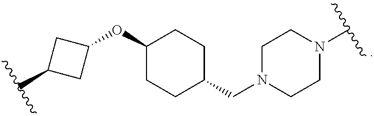 [SMILES: *N1CCN(C[C@H]2CC[C@H](O[C@H]3C[C@H](*)C3)CC2)CC1]

In some embodiments, L is

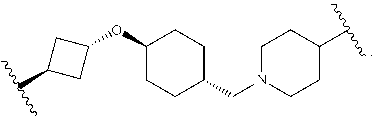 [SMILES: *C1CCN(C[C@H]2CC[C@H](O[C@H]3C[C@H](*)C3)CC2)CC1]

In some embodiments, L is

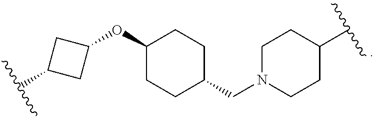 [SMILES: *C1CCN(C[C@H]2CC[C@H](O[C@H]3C[C@@H](*)C3)CC2)CC1]

In some embodiments, L is

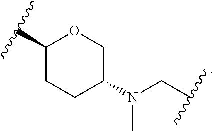 [SMILES: *CN(C)[C@@H]1CC[C@@H](*)OC1]

In some embodiments, L is

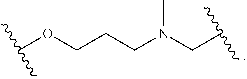 [SMILES: *CN(C)CCCO*]

In some embodiments, L is

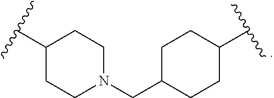 [SMILES: *C1CCC(CN2CCC(*)CC2)CC1]

In some embodiments, L is

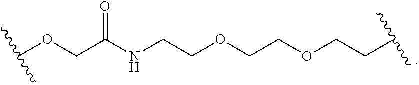 [SMILES: *CCOCCOCCNC(=O)CO*]

In some embodiments, L is

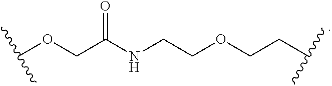 [SMILES: *CCOCCNC(=O)CO*]

In some embodiments, L is

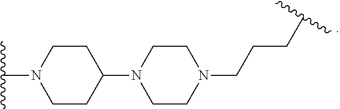 [SMILES: *CCCN1CCN(C2CCN(*)CC2)CC1]

In some embodiments, L is

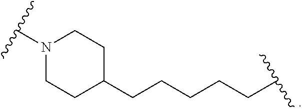 [SMILES: *CCCCCC1CCN(*)CC1]

In some embodiments, L is

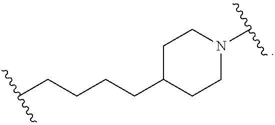 [SMILES: *CCCCC1CCN(*)CC1]

In some embodiments, L is

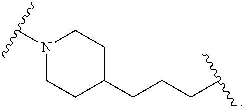 [SMILES: *CCCC1CCN(*)CC1]

In some embodiments, L is

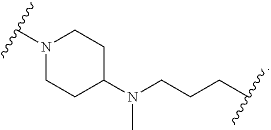 [SMILES: *CCCN(C)C1CCN(*)CC1]

In some embodiments, L is

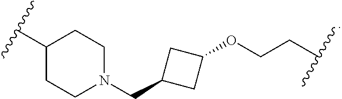 [SMILES: *CCO[C@H]1C[C@H](CN2CCC(*)CC2)C1]

In some embodiments, L is

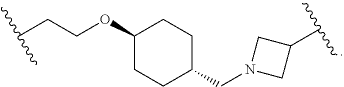 [SMILES: *CCO[C@H]1CC[C@H](CN2CC(*)C2)CC1]

In some embodiments, L is

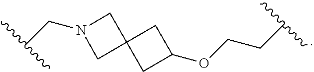 [SMILES: *CCOC1CC2(C1)CN(C*)C2]

In some embodiments, L is

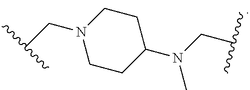 [SMILES: *CN1CCC(N(C)C*)CC1]

In some embodiments, L is

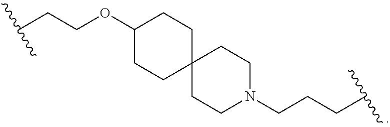 [SMILES: *CCCN1CCC2(CCC(OCC*)CC2)CC1]

In some embodiments, L is

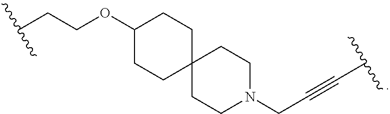 [SMILES: *C#CCN1CCC2(CCC(OCC*)CC2)CC1]

In some embodiments, L is

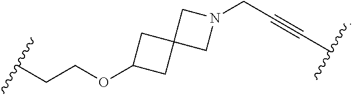 [SMILES: *C#CCN1CC2(CC(OCC*)C2)C1]

In some embodiments, L is

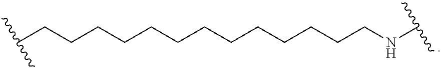 [SMILES: *CCCCCCCCCCCCCN*]

In some embodiments, L is

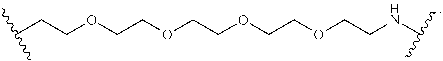 [SMILES: *CCOCCOCCOCCOCCN*]

In some embodiments, L is

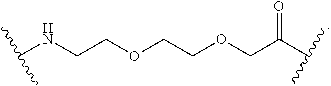 [SMILES: *NCCOCCOCC(*)=O]

In some embodiments, L is

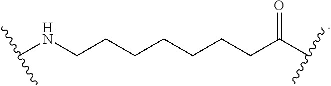 [SMILES: *NCCCCCCCC(*)=O]

In some embodiments, L is

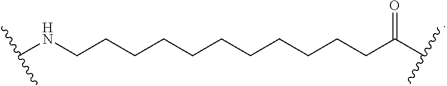 [SMILES: *NCCCCCCCCCCCC(*)=O]

In some embodiments, L is

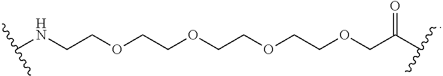 [SMILES: *NCCOCCOCCOCCOCC(*)=O]

In some embodiments, L is

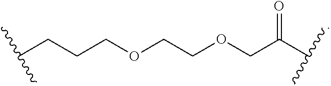 [SMILES: *CCCOCCOCC(*)=O]

In some embodiments, L is

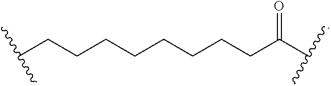 [SMILES: *CCCCCCCCC(*)=O]

In some embodiments, L is

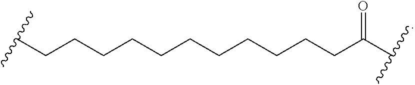 [SMILES: *CCCCCCCCCCCC(*)=O]

In some embodiments, L is

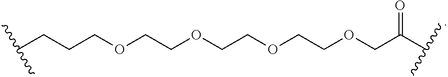 [SMILES: *CCCOCCOCCOCCOCC(*)=O]

In some embodiments, L is

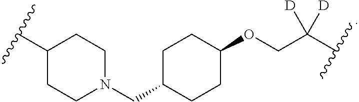 [SMILES: *C1CCN(C[C@H]2CC[C@H](OCC(*)([2H])[2H])CC2)CC1]

In some embodiments, L is

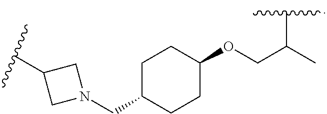 [SMILES: *C(C)CO[C@H]1CC[C@H](CN2CC(*)C2)CC1]

In some embodiments, L is

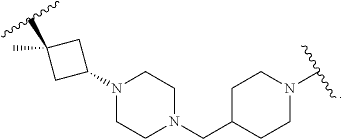 [SMILES: *N1CCC(CN2CCN([C@H]3C[C@@](*)(C)C3)CC2)CC1]

In some embodiments, L is

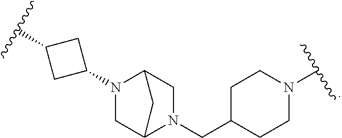 [SMILES: *N1CCC(CN2CC3CC2CN3[C@H]2C[C@@H](*)C2)CC1]

In some embodiments, L is

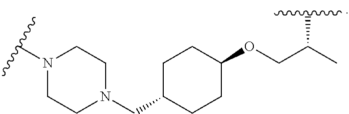 [SMILES: *[C@H](C)CO[C@H]1CC[C@H](CN2CCN(*)CC2)CC1]

In some embodiments, L is

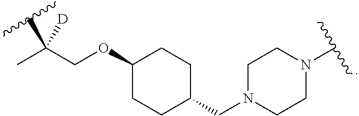 [SMILES: *N1CCN(C[C@H]2CC[C@H](OC[C@@](*)([2H])C)CC2)CC1]

In some embodiments, L is

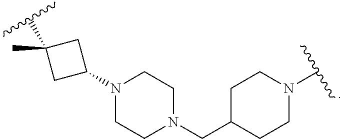 [SMILES: *N1CCC(CN2CCN([C@H]3C[C@](*)(C)C3)CC2)CC1]

In some embodiments, L is

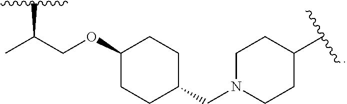 [SMILES: *C1CCN(C[C@H]2CC[C@H](OC[C@H](*)C)CC2)CC1]

In some embodiments, L is

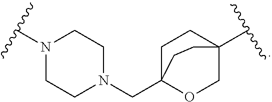 [SMILES: *N1CCN(CC23CCC(*)(CC2)CO3)CC1]

In some embodiments, L is

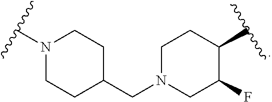 [SMILES: *[C@@H]1CCN(CC2CCN(*)CC2)C[C@@H]1F]

In some embodiments, L is

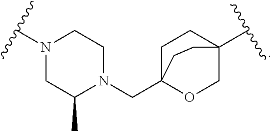 [SMILES: *N1CCN(CC23CCC(*)(CC2)CO3)[C@@H](C)C1]

In some embodiments, L is

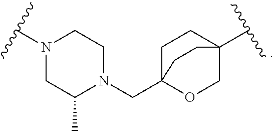 [SMILES: *N1CCN(CC23CCC(*)(CC2)CO3)[C@H](C)C1]

In some embodiments, L is

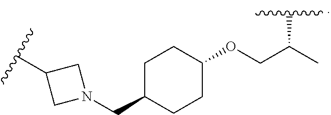 [SMILES: *C1CN(C[C@H]2CC[C@H](OC[C@H](*)C)CC2)C1]

In some embodiments, L is

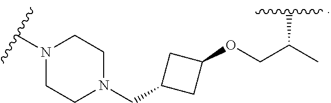 [SMILES: *[C@H](C)CO[C@H]1C[C@H](CN2CCN(*)CC2)C1]

In some embodiments, L is

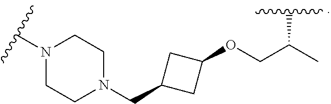 [SMILES: *[C@H](C)CO[C@H]1C[C@@H](CN2CCN(*)CC2)C1]

In some embodiments, L is

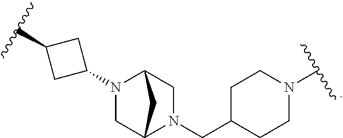 [SMILES: *N1CCC(CN2C[C@@H]3C[C@H]2CN3[C@H]2C[C@H](*)C2)CC1]

In some embodiments, L is

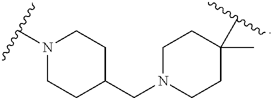 [SMILES: *N1CCC(CN2CCC(*)(C)CC2)CC1]

In some embodiments, L is

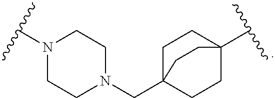 [SMILES: *N1CCN(CC23CCC(*)(CC2)CC3)CC1]

In some embodiments, L is

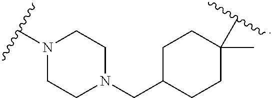 [SMILES: *N1CCN(CC2CCC(*)(C)CC2)CC1]

In some embodiments, L is

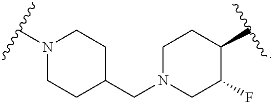 [SMILES: *[C@@H]1CCN(CC2CCN(*)CC2)C[C@H]1F]

In some embodiments, L is

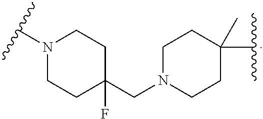 [SMILES: *N1CCC(F)(CN2CCC(*)(C)CC2)CC1]

In some embodiments, L is

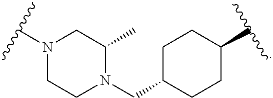 [SMILES: *N1CCN(C[C@H]2CC[C@H](*)CC2)[C@@H](C)C1]

In some embodiments, L is

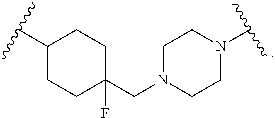 [SMILES: *C1CCC(F)(CN2CCN(*)CC2)CC1]

In some embodiments, L is

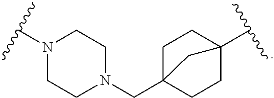 [SMILES: *N1CCN(CC23CCC(*)(CC2)C3)CC1]

In some embodiments, L is

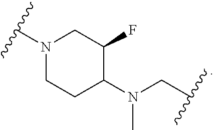 [SMILES: *CN(C)C1CCN(*)C[C@H]1F]

In some embodiments, L is

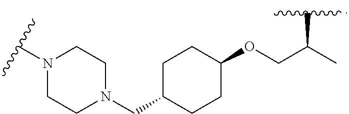 [SMILES: *[C@@H](C)CO[C@H]1CC[C@H](CN2CCN(*)CC2)CC1]

In some embodiments, L is

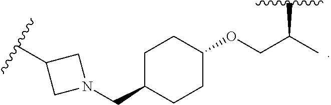 [SMILES: *C1CN(C[C@H]2CC[C@H](OC[C@@H](*)C)CC2)C1]

In some embodiments, L is

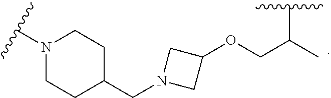 [SMILES: *C(C)COC1CN(CC2CCN(*)CC2)C1]

In some embodiments, L is

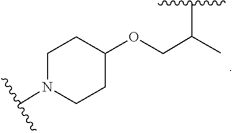 [SMILES: *C(C)COC1CCN(*)CC1]

In some embodiments, L is

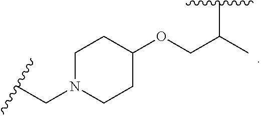 [SMILES: *CN1CCC(OCC(*)C)CC1]

In some embodiments, L is

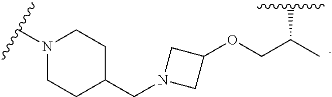 [SMILES: *[C@H](C)COC1CN(CC2CCN(*)CC2)C1]

In some embodiments, L is

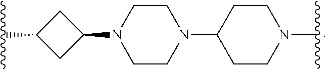 [SMILES: *N1CCC(N2CCN([C@H]3C[C@H](*)C3)CC2)CC1]

In some embodiments, L is

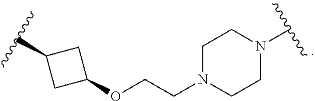 [SMILES: *N1CCN(CCO[C@H]2C[C@@H](*)C2)CC1]

In some embodiments, L is

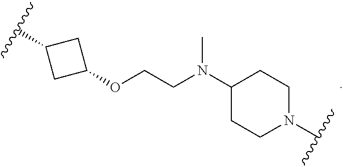 [SMILES: *N1CCC(N(C)CCO[C@H]2C[C@@H](*)C2)CC1]

In some embodiments, L is

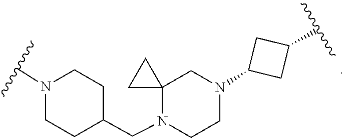 [SMILES: *N1CCC(CN2CCN([C@H]3C[C@@H](*)C3)CC23CC3)CC1]

In some embodiments, L is

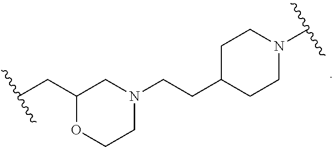 [SMILES: *CC1CN(CCC2CCN(*)CC2)CCO1]

In some embodiments, L is

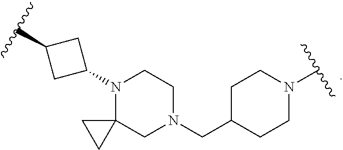 [SMILES: *N1CCC(CN2CCN([C@H]3C[C@H](*)C3)C3(CC3)C2)CC1]

In some embodiments, L is

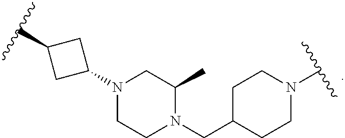 [SMILES: *N1CCC(CN2CCN([C@H]3C[C@H](*)C3)C[C@H]2C)CC1]

In some embodiments, L is

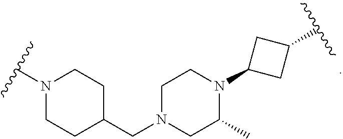 [SMILES: *N1CCC(CN2CCN([C@H]3C[C@H](*)C3)[C@H](C)C2)CC1]

In some embodiments, L is

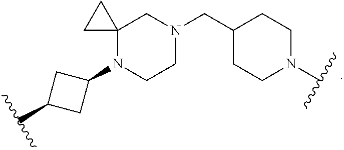 [SMILES: *N1CCC(CN2CCN([C@H]3C[C@@H](*)C3)C3(CC3)C2)CC1]

In some embodiments, L is

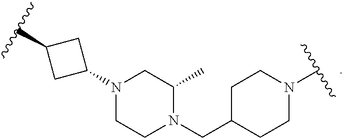 [SMILES: *N1CCC(CN2CCN([C@H]3C[C@H](*)C3)C[C@@H]2C)CC1]

In some embodiments, L is

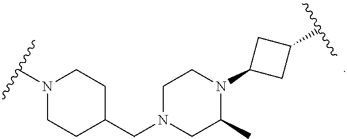 [SMILES: *N1CCC(CN2CCN([C@H]3C[C@H](*)C3)[C@@H](C)C2)CC1]

In some embodiments, L is

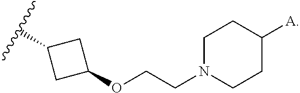

In some embodiments, L is

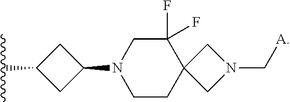

In some embodiments, L is

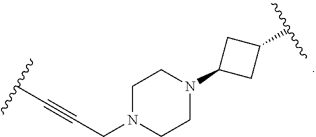 [SMILES: *C#CCN1CCN([C@H]2C[C@H](*)C2)CC1]

In some embodiments, L is

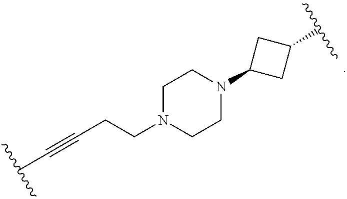 [SMILES: *C#CCCN1CCN([C@H]2C[C@H](*)C2)CC1]

In some embodiments, L is

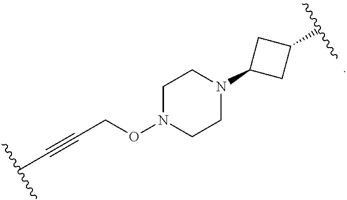 [SMILES: *C#CCON1CCN([C@H]2C[C@H](*)C2)CC1]

In some embodiments, L is

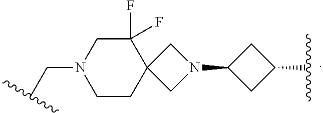 [SMILES: *CN1CCC2(CN([C@H]3C[C@H](*)C3)C2)C(F)(F)C1]

In some embodiments, L is

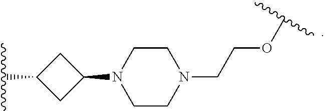 [SMILES: *OCCN1CCN([C@H]2C[C@H](*)C2)CC1]

In some embodiments, L is

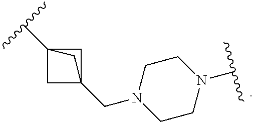 [SMILES: *N1CCN(CC23CC(*)(C2)C3)CC1]

In some embodiments, L is

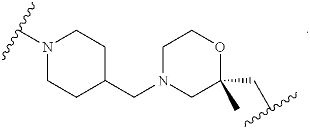 [SMILES: *C[C@]1(C)CN(CC2CCN(*)CC2)CCO1]

In some embodiments, L is

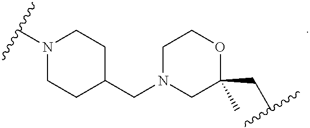 [SMILES: *C[C@@]1(C)CN(CC2CCN(*)CC2)CCO1]

In some embodiments, L is

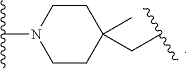 [SMILES: *CC1(C)CCN(*)CC1]

In some embodiments, L is

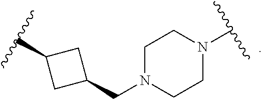 [SMILES: *N1CCN(C[C@H]2C[C@@H](*)C2)CC1]

In some embodiments, L is

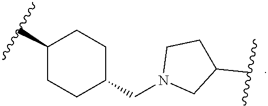 [SMILES: *C1CCN(C[C@H]2CC[C@H](*)CC2)C1]

In some embodiments, L is

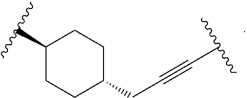 [SMILES: *C#CC[C@H]1CC[C@H](*)CC1]

In some embodiments, L is

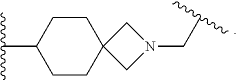 [SMILES: *CN1CC2(CCC(*)CC2)C1]

In some embodiments, L is

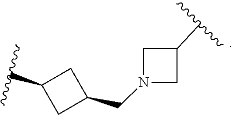 [SMILES: *C1CN(C[C@H]2C[C@@H](*)C2)C1]

In some embodiments, L is

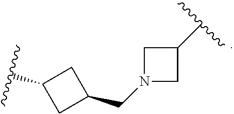 [SMILES: *C1CN(C[C@H]2C[C@H](*)C2)C1]

In some embodiments, L is

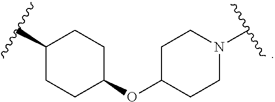 [SMILES: *N1CCC(O[C@H]2CC[C@@H](*)CC2)CC1]

In some embodiments, L is

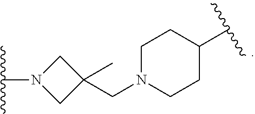 [SMILES: *C1CCN(CC2(C)CN(*)C2)CC1]

In some embodiments, L is

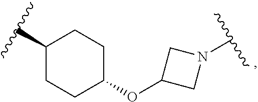 [SMILES: *N1CC(O[C@H]2CC[C@H](*)CC2)C1]

In some embodiments, L is

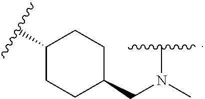 [SMILES: *N(C)C[C@H]1CC[C@H](*)CC1]

In some embodiments, L is

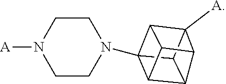

In some embodiments, L is

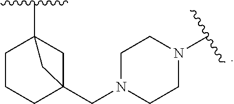 [SMILES: *N1CCN(CC23CCCC(*)(C2)C3)CC1]

In some embodiments, L is

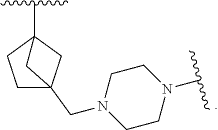 [SMILES: *N1CCN(CC23CCC(*)(C2)C3)CC1]

In some embodiments, L is

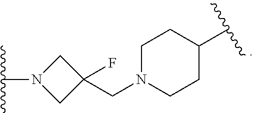 [SMILES: *C1CCN(CC2(F)CN(*)C2)CC1]

In some embodiments, L is

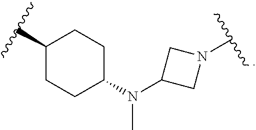 [SMILES: *N1CC(N(C)[C@H]2CC[C@H](*)CC2)C1]

In some embodiments, L is

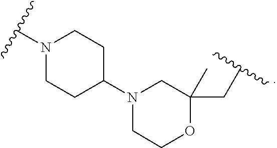 [SMILES: *CC1(C)CN(C2CCN(*)CC2)CCO1]

In some embodiments, L is

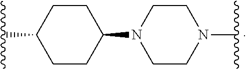 [SMILES: *N1CCN([C@H]2CC[C@H](*)CC2)CC1]

In some embodiments, L is

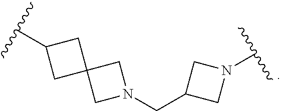 [SMILES: *C1CC2(C1)CN(CC1CN(*)C1)C2]

In some embodiments, L is

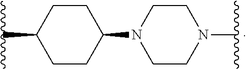 [SMILES: *N1CCN([C@H]2CC[C@@H](*)CC2)CC1]

In some embodiments, L is

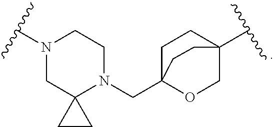 [SMILES: *N1CCN(CC23CCC(*)(CC2)CO3)C2(CC2)C1]

In some embodiments, L is

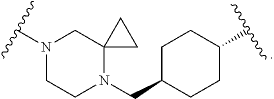 [SMILES: *N1CCN(C[C@H]2CC[C@H](*)CC2)C2(CC2)C1]

In some embodiments, L is

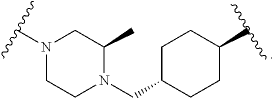 [SMILES: *N1CCN(C[C@H]2CC[C@H](*)CC2)[C@H](C)C1]

In some embodiments, L is

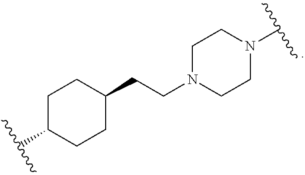 [SMILES: *N1CCN(CC[C@H]2CC[C@H](*)CC2)CC1]

In some embodiments, L is

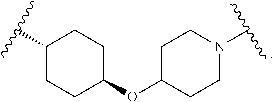 [SMILES: *N1CCC(O[C@H]2CC[C@H](*)CC2)CC1]

In some embodiments, L is

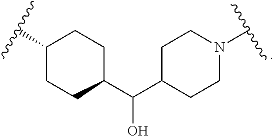 [SMILES: *N1CCC(C(O)[C@H]2CC[C@H](*)CC2)CC1]

In some embodiments, L is

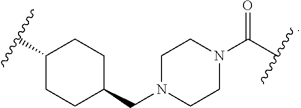 [SMILES: *C(=O)N1CCN(C[C@H]2CC[C@H](*)CC2)CC1]

In some embodiments, L is

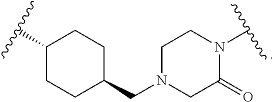 [SMILES: *N1CCN(C[C@H]2CC[C@H](*)CC2)CC1=O]

In some embodiments, L is

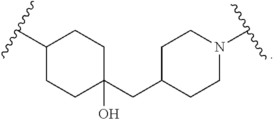 [SMILES: *C1CCC(O)(CC2CCN(*)CC2)CC1]

In some embodiments, L is

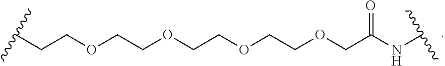 [SMILES: *CCOCCOCCOCCOCC(=O)N*]

In some embodiments, L is

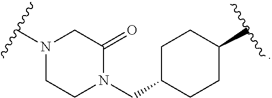 [SMILES: *N1CCN(C[C@H]2CC[C@H](*)CC2)C(=O)C1]

In some embodiments, L is

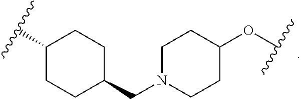 [SMILES: *OC1CCN(C[C@H]2CC[C@H](*)CC2)CC1]

In some embodiments, L is

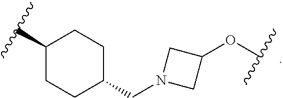 [SMILES: *OC1CN(C[C@H]2CC[C@H](*)CC2)C1]

In some embodiments, L is

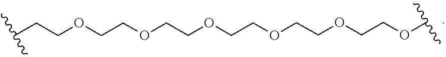 [SMILES: *CCOCCOCCOCCOCCOCCO*]

In some embodiments, L is

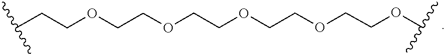 [SMILES: *CCOCCOCCOCCOCCO*]

In some embodiments, L is

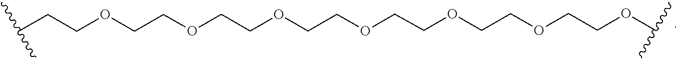 [SMILES: *CCOCCOCCOCCOCCOCCOCCO*]

In some embodiments, L is

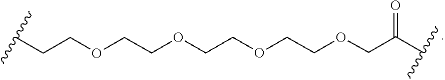 [SMILES: *CCOCCOCCOCCOCC(*)=O]

In some embodiments, L is

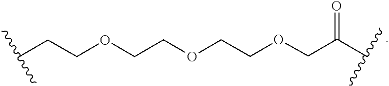 [SMILES: *CCOCCOCCOCC(*)=O]

In some embodiments, L is

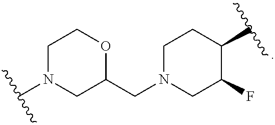 [SMILES: *[C@@H]1CCN(CC2CN(*)CCO2)C[C@@H]1F]

In some embodiments, L is

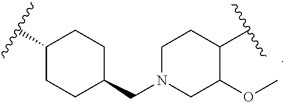 [SMILES: *C1CCN(C[C@H]2CC[C@H](*)CC2)CC1OC]

In some embodiments, L is

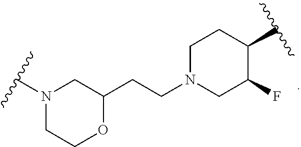 [SMILES: *[C@@H]1CCN(CCC2CN(*)CCO2)C[C@@H]1F]

In some embodiments, L is

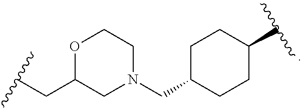 [SMILES: *CC1CN(C[C@H]2CC[C@H](*)CC2)CCO1]

In some embodiments, L is

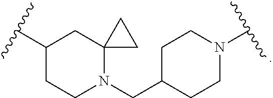 [SMILES: *C1CCN(CC2CCN(*)CC2)C2(CC2)C1]

In some embodiments, L is

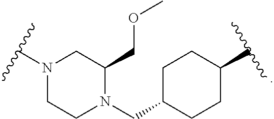 [SMILES: *N1CCN(C[C@H]2CC[C@H](*)CC2)[C@H](COC)C1]

In some embodiments, L is

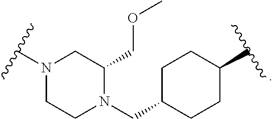 [SMILES: *N1CCN(C[C@H]2CC[C@H](*)CC2)[C@@H](COC)C1]

In some embodiments, L is

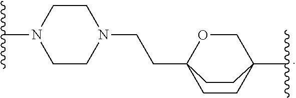 [SMILES: *N1CCN(CCC23CCC(*)(CC2)CO3)CC1]

In some embodiments, L is

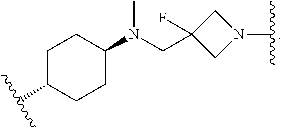 [SMILES: *N1CC(F)(CN(C)[C@H]2CC[C@H](*)CC2)C1]

In some embodiments, L is

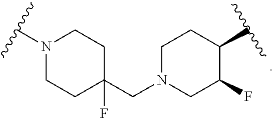 [SMILES: *[C@@H]1CCN(CC2(F)CCN(*)CC2)C[C@@H]1F]

In some embodiments, L is

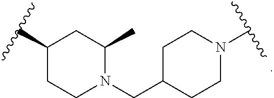 [SMILES: *[C@@H]1CCN(CC2CCN(*)CC2)[C@H](C)C1]

In some embodiments, L is

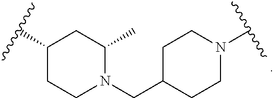 [SMILES: *[C@H]1CCN(CC2CCN(*)CC2)[C@@H](C)C1]

In some embodiments, L is

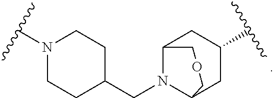

In some embodiments, L is

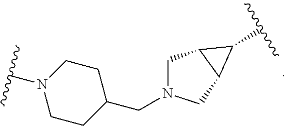 [SMILES: *[C@H]1[C@H]2CN(CC3CCN(*)CC3)C[C@@H]12]

In some embodiments, L is

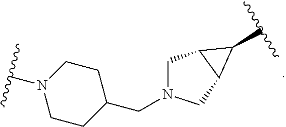 [SMILES: *[C@@H]1[C@H]2CN(CC3CCN(*)CC3)C[C@@H]12]

In some embodiments, L is

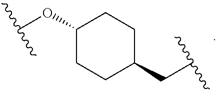 [SMILES: *C[C@H]1CC[C@H](O*)CC1]

In some embodiments, L is

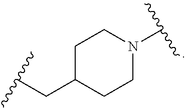 [SMILES: *CC1CCN(*)CC1]

In some embodiments, L is

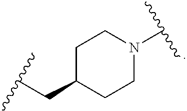

In some embodiments, L is

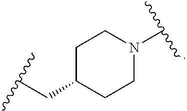

In some embodiments, L is

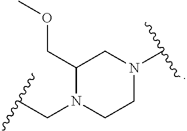 [SMILES: *CN1CCN(*)CC1COC]

In some embodiments, L is

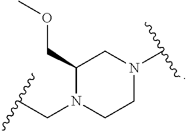 [SMILES: *CN1CCN(*)C[C@@H]1COC]

In some embodiments, L is

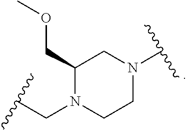 [SMILES: *CN1CCN(*)C[C@@H]1COC]

In some embodiments, L is

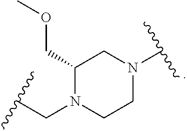 [SMILES: *CN1CCN(*)C[C@H]1COC]

In some embodiments, L is

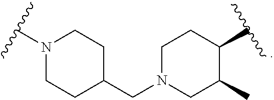 [SMILES: *[C@@H]1CCN(CC2CCN(*)CC2)C[C@@H]1C]

In some embodiments, L is

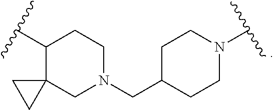 [SMILES: *C1CCN(CC2CCN(*)CC2)CC12CC2]

In some embodiments, L is

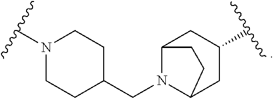

In some embodiments, L is

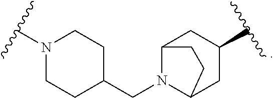

In some embodiments, L is

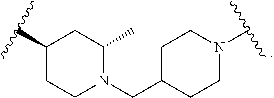 [SMILES: *[C@@H]1CCN(CC2CCN(*)CC2)[C@@H](C)C1]

In some embodiments, L is

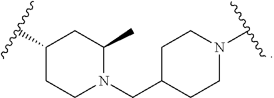 [SMILES: *[C@H]1CCN(CC2CCN(*)CC2)[C@H](C)C1]

In some embodiments, L is

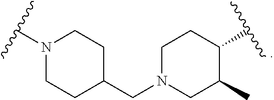 [SMILES: *[C@H]1CCN(CC2CCN(*)CC2)C[C@@H]1C]

In some embodiments, L is

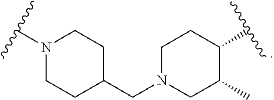 [SMILES: *[C@H]1CCN(CC2CCN(*)CC2)C[C@H]1C]

In some embodiments, L is

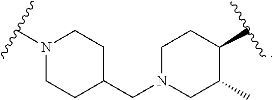 [SMILES: *[C@@H]1CCN(CC2CCN(*)CC2)C[C@H]1C]

In some embodiments, L is

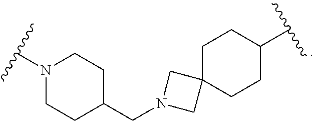 [SMILES: *C1CCC2(CC1)CN(CC1CCN(*)CC1)C2]

In some embodiments, L is

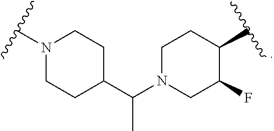 [SMILES: *[C@@H]1CCN(C(C)C2CCN(*)CC2)C[C@@H]1F]

In some embodiments, L is

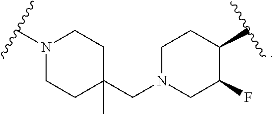 [SMILES: *[C@@H]1CCN(CC2(C)CCN(*)CC2)C[C@@H]1F]

In some embodiments, L is

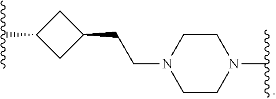 [SMILES: *N1CCN(CC[C@H]2C[C@H](*)C2)CC1]

In some embodiments, L is

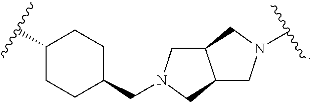 [SMILES: *N1C[C@@H]2CN(C[C@H]3CC[C@H](*)CC3)C[C@@H]2C1]

In some embodiments, L is

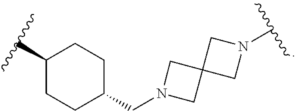 [SMILES: *N1CC2(C1)CN(C[C@H]1CC[C@H](*)CC1)C2]

In some embodiments, L is

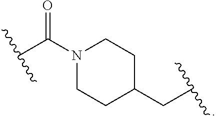 [SMILES: *CC1CCN(C(*)=O)CC1]

In some embodiments, L is

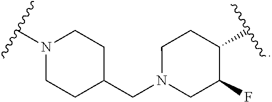 [SMILES: *[C@H]1CCN(CC2CCN(*)CC2)C[C@@H]1F]

In some embodiments, L is

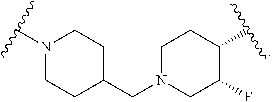 [SMILES: *[C@H]1CCN(CC2CCN(*)CC2)C[C@H]1F]

In some embodiments, L is

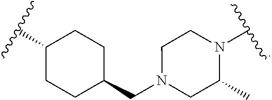 [SMILES: *N1CCN(C[C@H]2CC[C@H](*)CC2)C[C@H]1C]

In some embodiments, L is

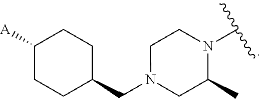

In some embodiments, L is

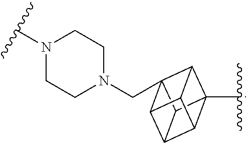 [SMILES: *N1CCN(CC23C4C5C6C4C2C6(*)C53)CC1]

In some embodiments, L is

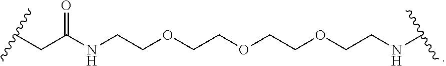 [SMILES: *CC(=O)NCCOCCOCCOCCN*]

In some embodiments, L is

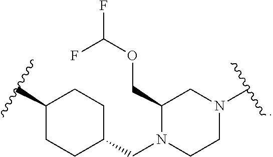 [SMILES: *N1CCN(C[C@H]2CC[C@H](*)CC2)[C@@H](COC(F)F)C1]

In some embodiments, L is

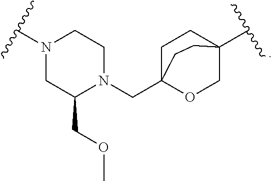 [SMILES: *N1CCN(CC23CCC(*)(CC2)CO3)[C@@H](COC)C1]

In some embodiments, L is

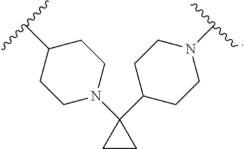 [SMILES: *C1CCN(C2(C3CCN(*)CC3)CC2)CC1]

In some embodiments, L is

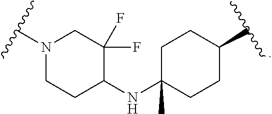 [SMILES: *N1CCC(N[C@]2(C)CC[C@@H](*)CC2)C(F)(F)C1]

In some embodiments, L is

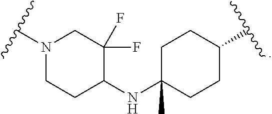 [SMILES: *N1CCC(N[C@]2(C)CC[C@H](*)CC2)C(F)(F)C1]

In some embodiments, L is

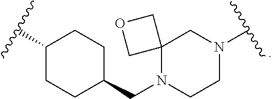 [SMILES: *N1CCN(C[C@H]2CC[C@H](*)CC2)C2(COC2)C1]

In some embodiments, L is

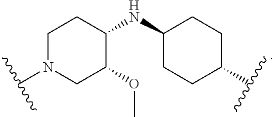 [SMILES: *N1CC[C@H](N[C@H]2CC[C@H](*)CC2)[C@H](OC)C1]

In some embodiments, L is

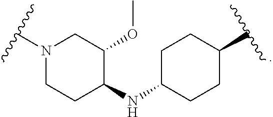 [SMILES: *N1CC[C@H](N[C@H]2CC[C@H](*)CC2)[C@@H](OC)C1]

In some embodiments, L is

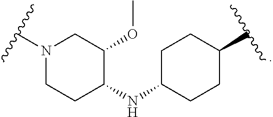 [SMILES: *N1CC[C@@H](N[C@H]2CC[C@H](*)CC2)[C@@H](OC)C1]

In some embodiments, L is

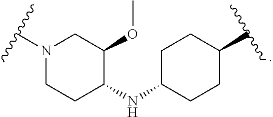 [SMILES: *N1CC[C@@H](N[C@H]2CC[C@H](*)CC2)[C@H](OC)C1]

In some embodiments, L is

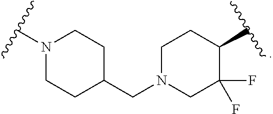 [SMILES: *[C@@H]1CCN(CC2CCN(*)CC2)CC1(F)F]

In some embodiments, L is

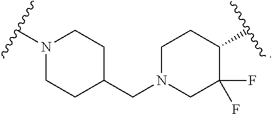 [SMILES: *[C@H]1CCN(CC2CCN(*)CC2)CC1(F)F]

In some embodiments, L is

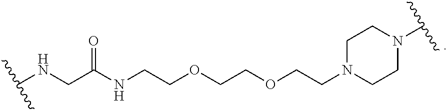 [SMILES: *NCC(=O)NCCOCCOCCN1CCN(*)CC1]

In some embodiments, L is

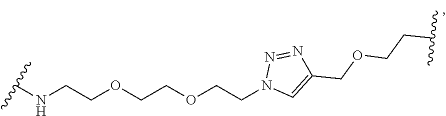 [SMILES: *CCOCc1cn(CCOCCOCCN*)nn1]

In some embodiments, L is

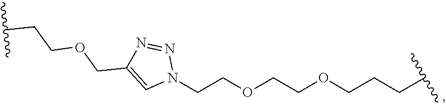 [SMILES: *CCCOCCOCCn1cc(COCC*)nn1]

In some embodiments, L is

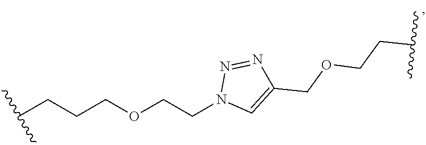 [SMILES: *CCCOCCn1cc(COCC*)nn1]

In some embodiments, L is

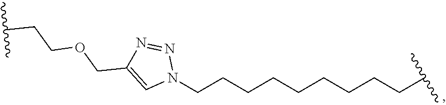 [SMILES: *CCCCCCCCCn1cc(COCC*)nn1]

In some embodiments, L is

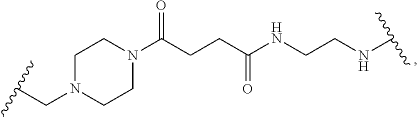 [SMILES: *CN1CCN(C(=O)CCC(=O)NCCN*)CC1]

In some embodiments, L is

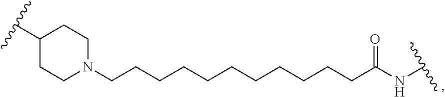 [SMILES: *NC(=O)CCCCCCCCCCCN1CCC(*)CC1]

In some embodiments, L is

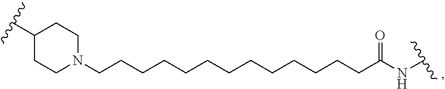 [SMILES: *NC(=O)CCCCCCCCCCCCCN1CCC(*)CC1]

In some embodiments, L is

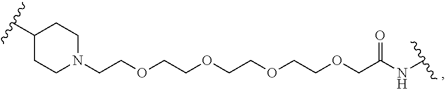 [SMILES: *NC(=O)COCCOCCOCCOCCN1CCC(*)CC1]

In some embodiments, L is

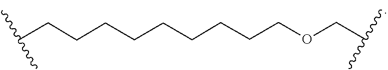 [SMILES: *CCCCCCCCCOC*]

In some embodiments, L is

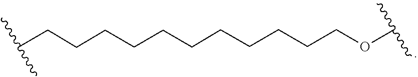 [SMILES: *CCCCCCCCCCCO*]

In some embodiments, L is

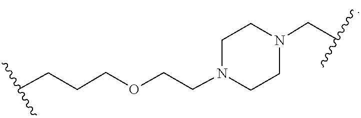 [SMILES: *CCCOCCN1CCN(C*)CC1]

In some embodiments, L is

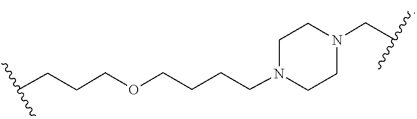 [SMILES: *CCCOCCCCN1CCN(C*)CC1]

In some embodiments, L is

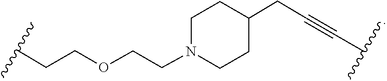 [SMILES: *C#CCC1CCN(CCOCC*)CC1]

In some embodiments, L is

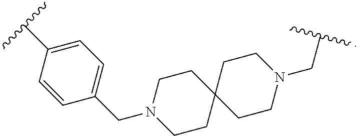 [SMILES: *CN1CCC2(CC1)CCN(Cc1ccc(*)cc1)CC2]

In some embodiments, L is

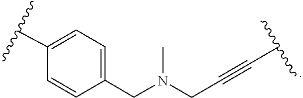 [SMILES: *C#CCN(C)Cc1ccc(*)cc1]

In some embodiments, L is

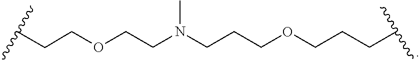 [SMILES: *CCCOCCCN(C)CCOCC*]

In some embodiments, L is

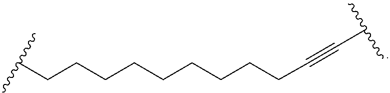 [SMILES: *C#CCCCCCCCCC*]

In some embodiments, L is

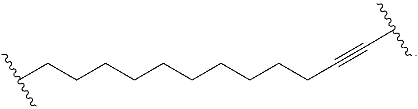 [SMILES: *C#CCCCCCCCCCC*]

In some embodiments, L is

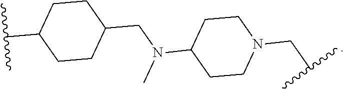 [SMILES: *CN1CCC(N(C)CC2CCC(*)CC2)CC1]

In some embodiments, L is

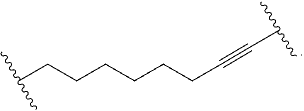 [SMILES: *C#CCCCCCC*]

In some embodiments, L is

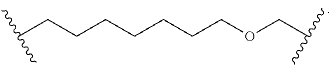 [SMILES: *CCCCCCCOC*]

In some embodiments, L is

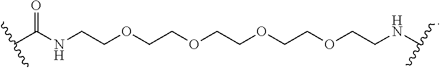 [SMILES: *NCCOCCOCCOCCOCCNC(*)=O]

In some embodiments, L is

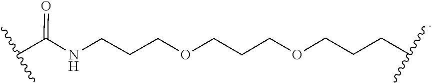 [SMILES: *CCCOCCCOCCCNC(*)=O]

In some embodiments, L is

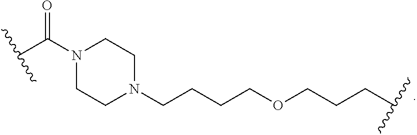 [SMILES: *CCCOCCCCN1CCN(C(*)=O)CC1]

In some embodiments, L is

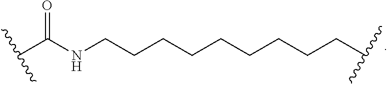 [SMILES: *CCCCCCCCCNC(*)=O]

In some embodiments, L is

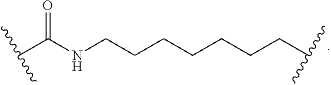 [SMILES: *CCCCCCCNC(*)=O]

In some embodiments, L is

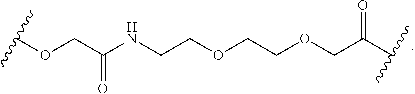 [SMILES: *OCC(=O)NCCOCCOCC(*)=O]

In some embodiments, L is

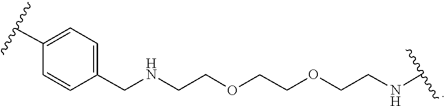 [SMILES: *NCCOCCOCCNCc1ccc(*)cc1]

In some embodiments, L is

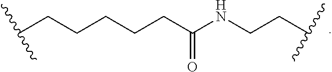 [SMILES: *CCCCCC(=O)NCC*]

In some embodiment, L is also selected from those depicted in Table B, below.

In some embodiments, L is selected from those depicted in Table 1, below.

Without limitation, the point of attachment of L to CBM and DIM can be, for example when L is

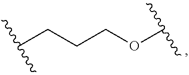 [SMILES: *CCCO*]

either

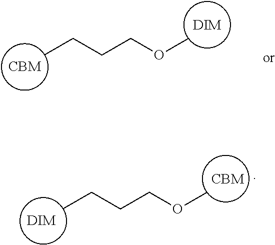

In some embodiments, a provided compound or pharmaceutically acceptable salt thereof, is selected from those wherein CBM is

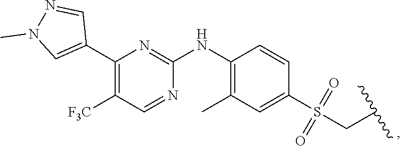 [SMILES: *CS(=O)(=O)c1ccc(Nc2ncc(C(F)(F)F)c(-c3cnn(C)c3)n2)c(C)c1]

LBM is selected from any of those in Table A below, and L is selected from any of those in Table B below.

In some embodiments, a provided compound or pharmaceutically acceptable salt thereof, is selected from those wherein CBM is

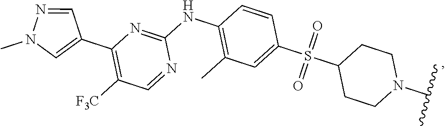 [SMILES: *N1CCC(S(=O)(=O)c2ccc(Nc3ncc(C(F)(F)F)c(-c4cnn(C)c4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

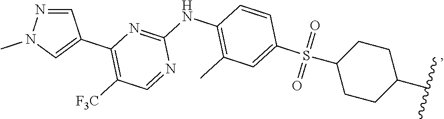 [SMILES: *C1CCC(S(=O)(=O)c2ccc(Nc3ncc(C(F)(F)F)c(-c4cnn(C)c4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

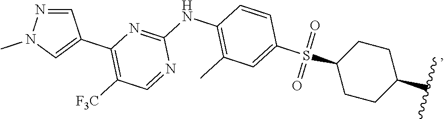 [SMILES: *[C@H]1CC[C@@H](S(=O)(=O)c2ccc(Nc3ncc(C(F)(F)F)c(-c4cnn(C)c4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

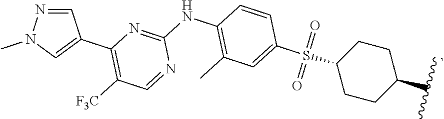 [SMILES: *[C@H]1CC[C@H](S(=O)(=O)c2ccc(Nc3ncc(C(F)(F)F)c(-c4cnn(C)c4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

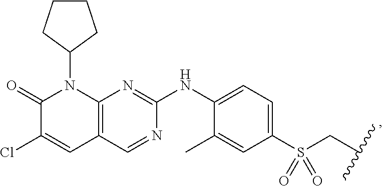 [SMILES: *CS(=O)(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

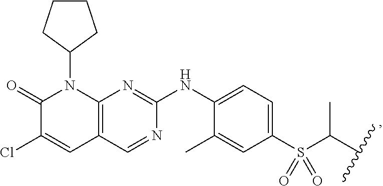 [SMILES: *C(C)S(=O)(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

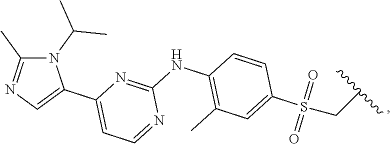 [SMILES: *CS(=O)(=O)c1ccc(Nc2nccc(-c3cnc(C)n3C(C)C)n2)c(C)c1]

LBM is selected from any of those in Table A below, and L is selected from any of those in Table B below.

In some embodiments, a provided compound or pharmaceutically acceptable salt thereof, is selected from those wherein CBM is

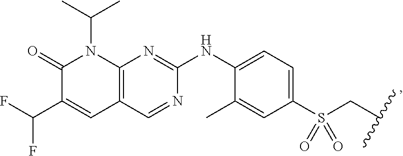 [SMILES: *CS(=O)(=O)c1ccc(Nc2ncc3cc(C(F)F)c(=O)n(C(C)C)c3n2)c(C)c1]

LBM is selected from any of those in Table A below, and L is selected from any of those in Table B below.

In some embodiments, a provided compound or pharmaceutically acceptable salt thereof, is selected from those wherein CBM is

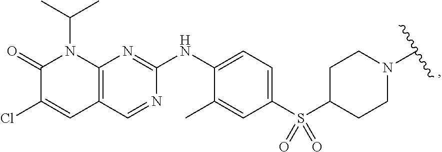 [SMILES: *N1CCC(S(=O)(=O)c2ccc(Nc3ncc4cc(Cl)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

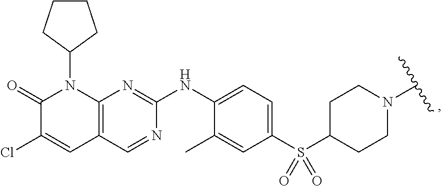 [SMILES: *N1CCC(S(=O)(=O)c2ccc(Nc3ncc4cc(Cl)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

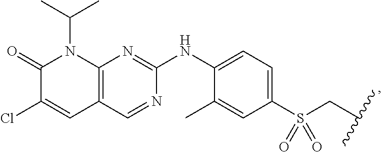 [SMILES: *CS(=O)(=O)c1ccc(Nc2ncc3cc(Cl)c(=O)n(C(C)C)c3n2)c(C)c1]

LBM is selected from any of those in Table A below, and L is selected from any of those in Table B below.

In some embodiments, a provided compound or pharmaceutically acceptable salt thereof, is selected from those wherein CBM is

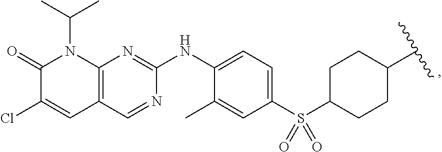 [SMILES: *C1CCC(S(=O)(=O)c2ccc(Nc3ncc4cc(Cl)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

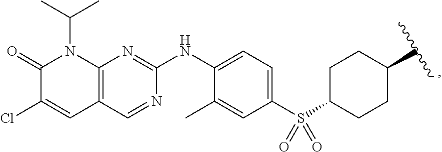 [SMILES: *[C@H]1CC[C@H](S(=O)(=O)c2ccc(Nc3ncc4cc(Cl)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

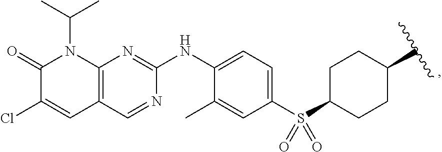 [SMILES: *[C@H]1CC[C@@H](S(=O)(=O)c2ccc(Nc3ncc4cc(Cl)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

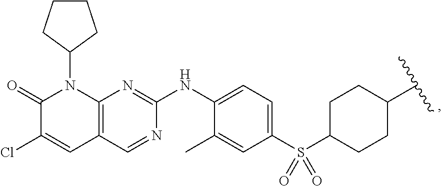 [SMILES: *C1CCC(S(=O)(=O)c2ccc(Nc3ncc4cc(Cl)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

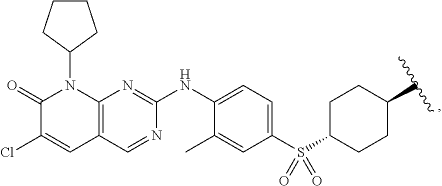 [SMILES: *[C@H]1CC[C@H](S(=O)(=O)c2ccc(Nc3ncc4cc(Cl)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

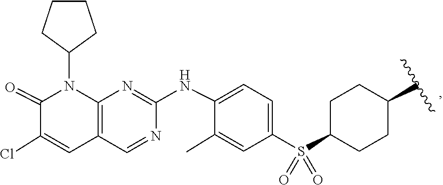 [SMILES: *[C@H]1CC[C@@H](S(=O)(=O)c2ccc(Nc3ncc4cc(Cl)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

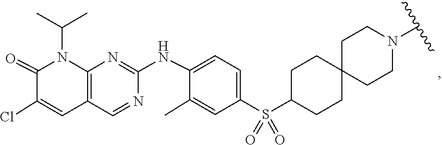 [SMILES: *N1CCC2(CCC(S(=O)(=O)c3ccc(Nc4ncc5cc(Cl)c(=O)n(C(C)C)c5n4)c(C)c3)CC2)CC1]

LBM is selected from any of those in Table A below, and L is selected from any of those in Table B below.

In some embodiments, a provided compound or pharmaceutically acceptable salt thereof, is selected from those wherein CBM is

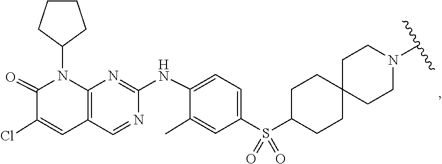 [SMILES: *N1CCC2(CCC(S(=O)(=O)c3ccc(Nc4ncc5cc(Cl)c(=O)n(C6CCCC6)c5n4)c(C)c3)CC2)CC1]

LBM is selected from any of those in Table A below, and L is selected from any of those in Table B below.

In some embodiments, a provided compound or pharmaceutically acceptable salt thereof, is selected from those wherein CBM is

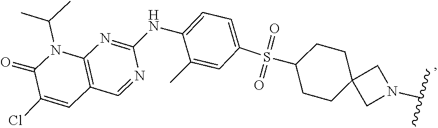 [SMILES: *N1CC2(CCC(S(=O)(=O)c3ccc(Nc4ncc5cc(Cl)c(=O)n(C(C)C)c5n4)c(C)c3)CC2)C1]

LBM is selected from any of those in Table A below, and L is selected from any of those in Table B below.

In some embodiments, a provided compound or pharmaceutically acceptable salt thereof, is selected from those wherein CBM is

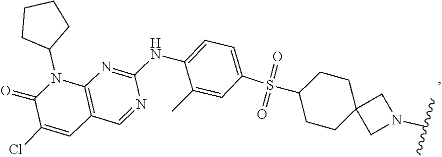 [SMILES: *N1CC2(CCC(S(=O)(=O)c3ccc(Nc4ncc5cc(Cl)c(=O)n(C6CCCC6)c5n4)c(C)c3)CC2)C1]

LBM is selected from any of those in Table A below, and L is selected from any of those in Table B below.

In some embodiments, a provided compound or pharmaceutically acceptable salt thereof, is selected from those wherein CBM is

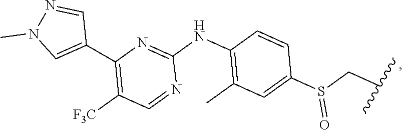 [SMILES: *CS(=O)c1ccc(Nc2ncc(C(F)(F)F)c(-c3cnn(C)c3)n2)c(C)c1]

LBM is selected from any of those in Table A below, and L is selected from any of those in Table B below.

In some embodiments, a provided compound or pharmaceutically acceptable salt thereof, is selected from those wherein CBM is

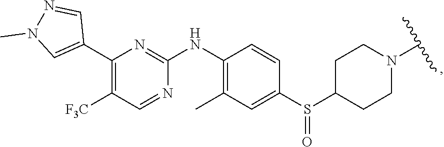 [SMILES: *N1CCC(S(=O)c2ccc(Nc3ncc(C(F)(F)F)c(-c4cnn(C)c4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

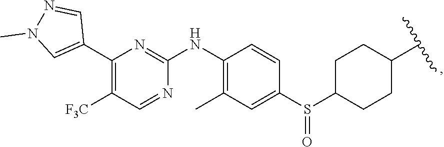 [SMILES: *C1CCC(S(=O)c2ccc(Nc3ncc(C(F)(F)F)c(-c4cnn(C)c4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

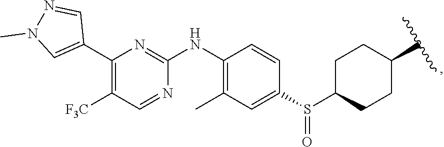 [SMILES: *[C@H]1CC[C@@H]([S@](=O)c2ccc(Nc3ncc(C(F)(F)F)c(-c4cnn(C)c4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

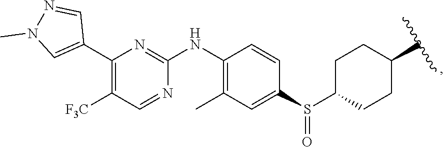 [SMILES: *[C@H]1CC[C@H]([S@@](=O)c2ccc(Nc3ncc(C(F)(F)F)c(-c4cnn(C)c4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

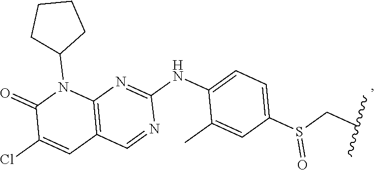 [SMILES: *CS(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

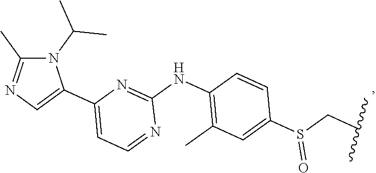 [SMILES: *CS(=O)c1ccc(Nc2nccc(-c3cnc(C)n3C(C)C)n2)c(C)c1]

LBM is selected from any of those in Table A below, and L is selected from any of those in Table B below.

In some embodiments, a provided compound or pharmaceutically acceptable salt thereof, is selected from those wherein CBM is

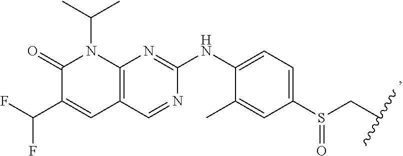 [SMILES: *CS(=O)c1ccc(Nc2ncc3cc(C(F)F)c(=O)n(C(C)C)c3n2)c(C)c1]

LBM is selected from any of those in Table A below, and L is selected from any of those in Table B below.

In some embodiments, a provided compound or pharmaceutically acceptable salt thereof, is selected from those wherein CBM is

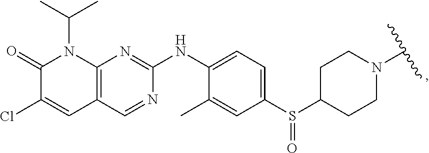 [SMILES: *N1CCC(S(=O)c2ccc(Nc3ncc4cc(Cl)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

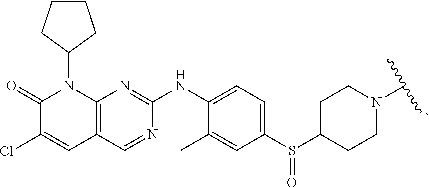 [SMILES: *N1CCC(S(=O)c2ccc(Nc3ncc4cc(Cl)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

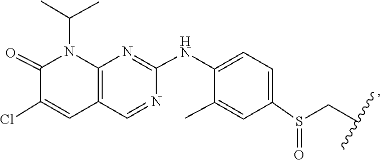 [SMILES: *CS(=O)c1ccc(Nc2ncc3cc(Cl)c(=O)n(C(C)C)c3n2)c(C)c1]

LBM is selected from any of those in Table A below, and L is selected from any of those in Table B below.

In some embodiments, a provided compound or pharmaceutically acceptable salt thereof, is selected from those wherein CBM is

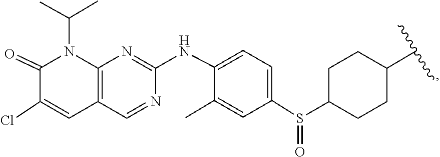 [SMILES: *C1CCC(S(=O)c2ccc(Nc3ncc4cc(Cl)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

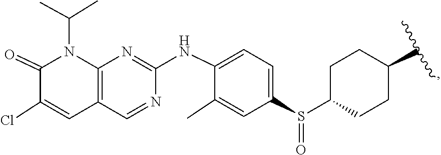 [SMILES: *[C@H]1CC[C@H]([S@@](=O)c2ccc(Nc3ncc4cc(Cl)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

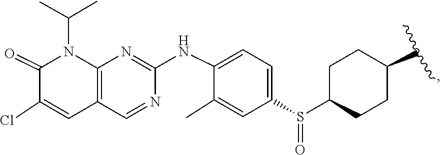 [SMILES: *[C@H]1CC[C@@H]([S@](=O)c2ccc(Nc3ncc4cc(Cl)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

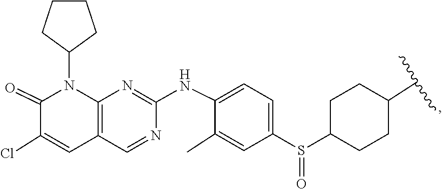 [SMILES: *C1CCC(S(=O)c2ccc(Nc3ncc4cc(Cl)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

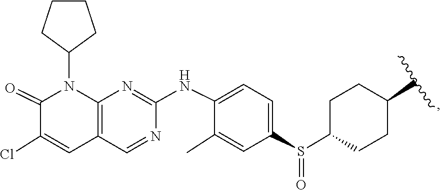 [SMILES: *[C@H]1CC[C@H]([S@@](=O)c2ccc(Nc3ncc4cc(Cl)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

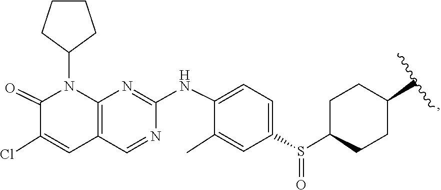 [SMILES: *[C@H]1CC[C@@H]([S@](=O)c2ccc(Nc3ncc4cc(Cl)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

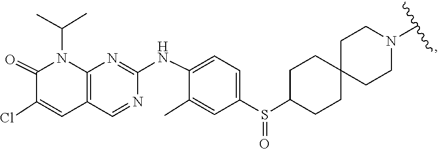 [SMILES: *N1CCC2(CCC(S(=O)c3ccc(Nc4ncc5cc(Cl)c(=O)n(C(C)C)c5n4)c(C)c3)CC2)CC1]

LBM is selected from any of those in Table A below, and L is selected from any of those in Table B below.

In some embodiments, a provided compound or pharmaceutically acceptable salt thereof, is selected from those wherein CBM is

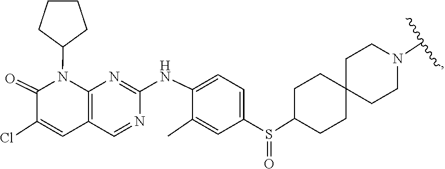 [SMILES: *N1CCC2(CCC(S(=O)c3ccc(Nc4ncc5cc(Cl)c(=O)n(C6CCCC6)c5n4)c(C)c3)CC2)CC1]

LBM is selected from any of those in Table A below, and L is selected from any of those in Table B below.

In some embodiments, a provided compound or pharmaceutically acceptable salt thereof, is selected from those wherein CBM is

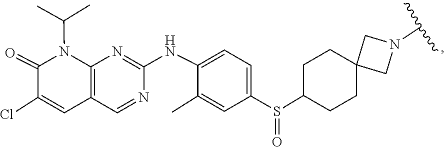 [SMILES: *N1CC2(CCC(S(=O)c3ccc(Nc4ncc5cc(Cl)c(=O)n(C(C)C)c5n4)c(C)c3)CC2)C1]

LBM is selected from any of those in Table A below, and L is selected from any of those in Table B below.

In some embodiments, a provided compound or pharmaceutically acceptable salt thereof, is selected from those wherein CBM is

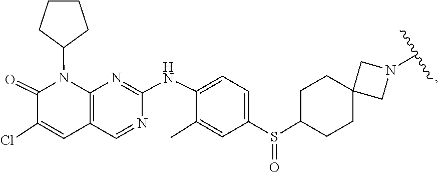 [SMILES: *N1CC2(CCC(S(=O)c3ccc(Nc4ncc5cc(Cl)c(=O)n(C6CCCC6)c5n4)c(C)c3)CC2)C1]

LBM is selected from any of those in Table A below, and L is selected from any of those in Table B below.

In some embodiments, a provided compound or pharmaceutically acceptable salt thereof, is selected from those wherein CBM is

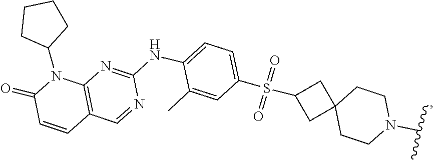 [SMILES: *N1CCC2(CC1)CC(S(=O)(=O)c1ccc(Nc3ncc4ccc(=O)n(C5CCCC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

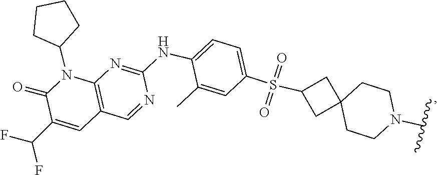 [SMILES: *N1CCC2(CC1)CC(S(=O)(=O)c1ccc(Nc3ncc4cc(C(F)F)c(=O)n(C5CCCC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

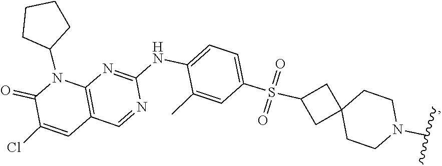 [SMILES: *N1CCC2(CC1)CC(S(=O)(=O)c1ccc(Nc3ncc4cc(Cl)c(=O)n(C5CCCC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

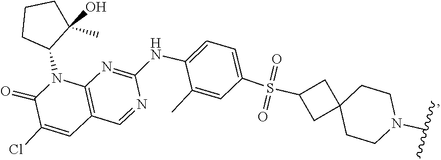 [SMILES: *N1CCC2(CC1)CC(S(=O)(=O)c1ccc(Nc3ncc4cc(Cl)c(=O)n([C@@H]5CCC[C@@]5(C)O)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

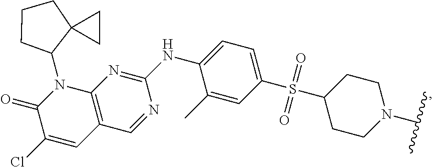 [SMILES: *N1CCC(S(=O)(=O)c2ccc(Nc3ncc4cc(Cl)c(=O)n(C5CCCC56CC6)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

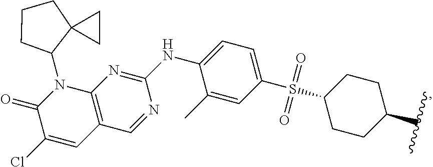 [SMILES: *[C@H]1CC[C@H](S(=O)(=O)c2ccc(Nc3ncc4cc(Cl)c(=O)n(C5CCCC56CC6)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

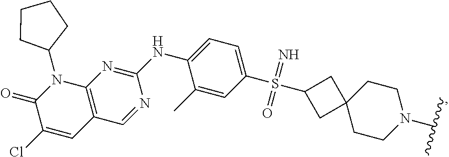 [SMILES: *N1CCC2(CC1)CC(S(=N)(=O)c1ccc(Nc3ncc4cc(Cl)c(=O)n(C5CCCC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

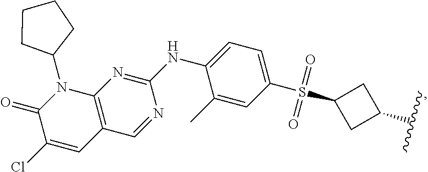 [SMILES: *[C@H]1C[C@H](S(=O)(=O)c2ccc(Nc3ncc4cc(Cl)c(=O)n(C5CCCC5)c4n3)c(C)c2)C1]

LBM is selected from any of those in Table A below, and L is selected from any of those in Table B below.

In some embodiments, a provided compound or pharmaceutically acceptable salt thereof, is selected from those wherein CBM is

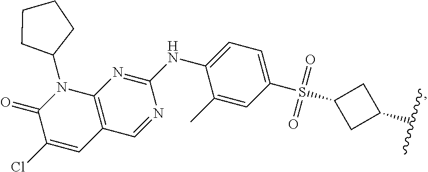 [SMILES: *[C@H]1C[C@@H](S(=O)(=O)c2ccc(Nc3ncc4cc(Cl)c(=O)n(C5CCCC5)c4n3)c(C)c2)C1]

LBM is selected from any of those in Table A below, and L is selected from any of those in Table B below.

In some embodiments, a provided compound or pharmaceutically acceptable salt thereof, is selected from those wherein CBM is

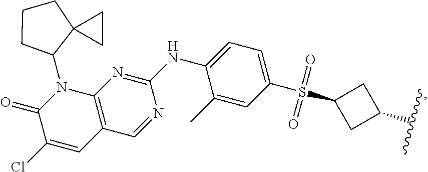 [SMILES: *[C@H]1C[C@H](S(=O)(=O)c2ccc(Nc3ncc4cc(Cl)c(=O)n(C5CCCC56CC6)c4n3)c(C)c2)C1]

LBM is selected from any of those in Table A below, and L is selected from any of those in Table B below.

In some embodiments, a provided compound or pharmaceutically acceptable salt thereof, is selected from those wherein CBM is

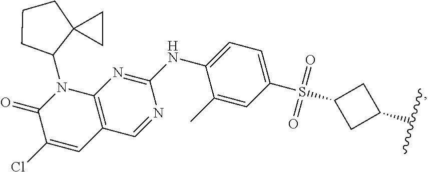 [SMILES: *[C@H]1C[C@@H](S(=O)(=O)c2ccc(Nc3ncc4cc(Cl)c(=O)n(C5CCCC56CC6)c4n3)c(C)c2)C1]

LBM is selected from any of those in Table A below, and L is selected from any of those in Table B below.

In some embodiments, a provided compound or pharmaceutically acceptable salt thereof, is selected from those wherein CBM is

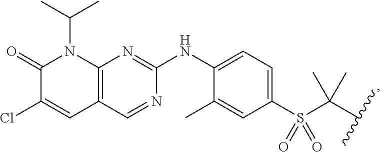 [SMILES: *C(C)(C)S(=O)(=O)c1ccc(Nc2ncc3cc(Cl)c(=O)n(C(C)C)c3n2)c(C)c1]

LBM is selected from any of those in Table A below, and L is selected from any of those in Table B below.

In some embodiments, a provided compound or pharmaceutically acceptable salt thereof, is selected from those wherein CBM is

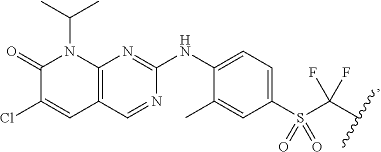 [SMILES: *C(F)(F)S(=O)(=O)c1ccc(Nc2ncc3cc(Cl)c(=O)n(C(C)C)c3n2)c(C)c1]

LBM is selected from any of those in Table A below, and L is selected from any of those in Table B below.

In some embodiments, a provided compound or pharmaceutically acceptable salt thereof, is selected from those wherein CBM is

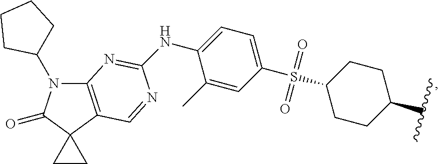 [SMILES: *[C@H]1CC[C@H](S(=O)(=O)c2ccc(Nc3ncc4c(n3)N(C3CCCC3)C(=O)C43CC3)c(C)c2)CC1]

LBM is selected from any of those in Table A below, and L is selected from any of those in Table B below.

In some embodiments, a provided compound or pharmaceutically acceptable salt thereof, is selected from those wherein CBM is

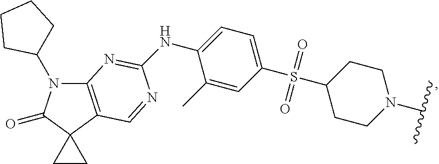 [SMILES: *N1CCC(S(=O)(=O)c2ccc(Nc3ncc4c(n3)N(C3CCCC3)C(=O)C43CC3)c(C)c2)CC1]

LBM is selected from any of those in Table A below, and L is selected from any of those in Table B below.

In some embodiments, a provided compound or pharmaceutically acceptable salt thereof, is selected from those wherein CBM is

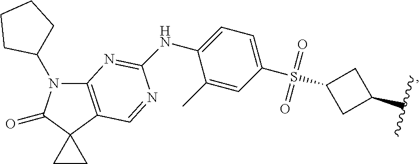 [SMILES: *[C@H]1C[C@H](S(=O)(=O)c2ccc(Nc3ncc4c(n3)N(C3CCCC3)C(=O)C43CC3)c(C)c2)C1]

LBM is selected from any of those in Table A below, and L is selected from any of those in Table B below.

In some embodiments, a provided compound or pharmaceutically acceptable salt thereof, is selected from those wherein CBM is

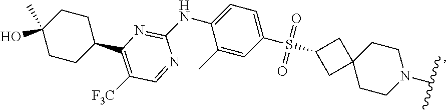

LBM is selected from any of those in Table A below, and L is selected from any of those in Table B below.

In some embodiments, a provided compound or pharmaceutically acceptable salt thereof, is selected from those wherein CBM is

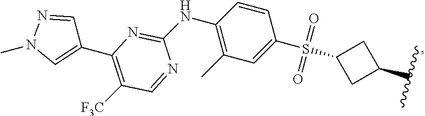 [SMILES: *[C@H]1C[C@H](S(=O)(=O)c2ccc(Nc3ncc(C(F)(F)F)c(-c4cnn(C)c4)n3)c(C)c2)C1]

LBM is selected from any of those in Table A below, and L is selected from any of those in Table B below.

In some embodiments, a provided compound or pharmaceutically acceptable salt thereof, is selected from those wherein CBM is

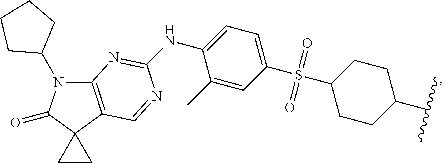 [SMILES: *C1CCC(S(=O)(=O)c2ccc(Nc3ncc4c(n3)N(C3CCCC3)C(=O)C43CC3)c(C)c2)CC1]

LBM is selected from any of those in Table A below, and L is selected from any of those in Table B below.

In some embodiments, a provided compound or pharmaceutically acceptable salt thereof, is selected from those wherein CBM is

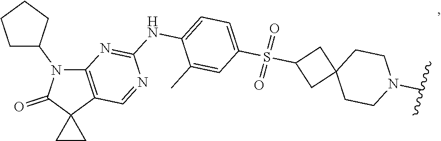 [SMILES: *N1CCC2(CC1)CC(S(=O)(=O)c1ccc(Nc3ncc4c(n3)N(C3CCCC3)C(=O)C43CC3)c(C)c1)C2]

LBM is selected from any of those in Table A below, and L is selected from any of those in Table B below.

In some embodiments, a provided compound or pharmaceutically acceptable salt thereof, is selected from those wherein CBM is

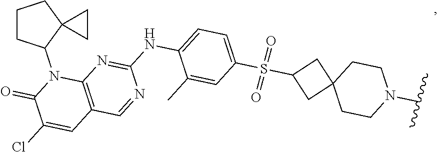 [SMILES: *N1CCC2(CC1)CC(S(=O)(=O)c1ccc(Nc3ncc4cc(Cl)c(=O)n(C5CCCC56CC6)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

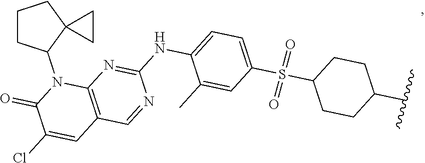 [SMILES: *C1CCC(S(=O)(=O)c2ccc(Nc3ncc4cc(Cl)c(=O)n(C5CCCC56CC6)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

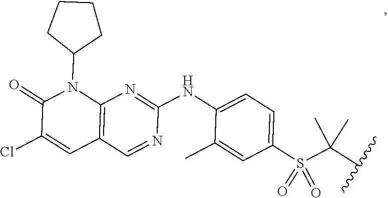 [SMILES: *C(C)(C)S(=O)(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

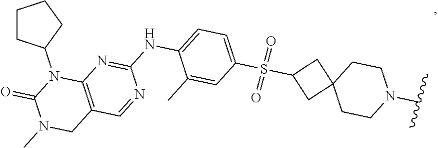 [SMILES: *N1CCC2(CC1)CC(S(=O)(=O)c1ccc(Nc3ncc4c(n3)N(C3CCCC3)C(=O)N(C)C4)c(C)c1)C2]

LBM is selected from any of those in Table A below, and L is selected from any of those in Table B below.

In some embodiments, a provided compound or pharmaceutically acceptable salt thereof, is selected from those wherein CBM is

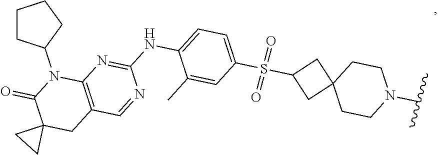 [SMILES: *N1CCC2(CC1)CC(S(=O)(=O)c1ccc(Nc3ncc4c(n3)N(C3CCCC3)C(=O)C3(CC3)C4)c(C)c1)C2]

LBM is selected from any of those in Table A below, and L is selected from any of those in Table B below.

In some embodiments, a provided compound or pharmaceutically acceptable salt thereof, is selected from those wherein CBM is

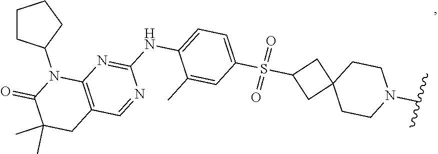 [SMILES: *N1CCC2(CC1)CC(S(=O)(=O)c1ccc(Nc3ncc4c(n3)N(C3CCCC3)C(=O)C(C)(C)C4)c(C)c1)C2]

LBM is selected from any of those in Table A below, and L is selected from any of those in Table B below.

In some embodiments, a provided compound or pharmaceutically acceptable salt thereof, is selected from those wherein CBM is

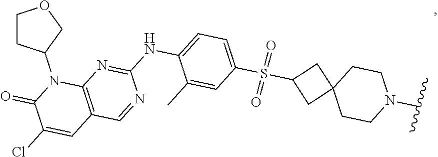 [SMILES: *N1CCC2(CC1)CC(S(=O)(=O)c1ccc(Nc3ncc4cc(Cl)c(=O)n(C5CCOC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

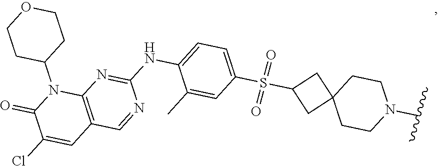 [SMILES: *N1CCC2(CC1)CC(S(=O)(=O)c1ccc(Nc3ncc4cc(Cl)c(=O)n(C5CCOCC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

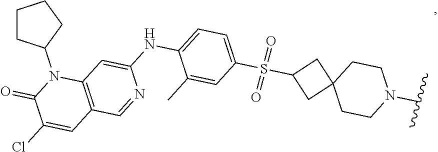 [SMILES: *N1CCC2(CC1)CC(S(=O)(=O)c1ccc(Nc3cc4c(cn3)cc(Cl)c(=O)n4C3CCCC3)c(C)c1)C2]

LBM is selected from any of those in Table A below, and L is selected from any of those in Table B below.

In some embodiments, a provided compound or pharmaceutically acceptable salt thereof, is selected from those wherein CBM is

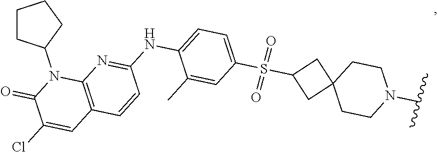 [SMILES: *N1CCC2(CC1)CC(S(=O)(=O)c1ccc(Nc3ccc4cc(Cl)c(=O)n(C5CCCC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

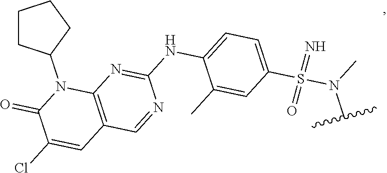 [SMILES: *N(C)S(=N)(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

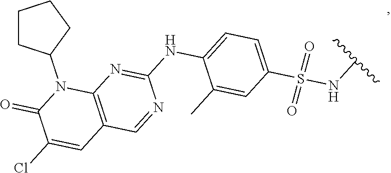 [SMILES: *NS(=O)(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

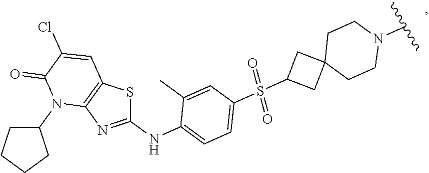 [SMILES: *N1CCC2(CC1)CC(S(=O)(=O)c1ccc(Nc3nc4c(cc(Cl)c(=O)n4C4CCCC4)s3)c(C)c1)C2]

LBM is selected from any of those in Table A below, and L is selected from any of those in Table B below.

In some embodiments, a provided compound or pharmaceutically acceptable salt thereof, is selected from those wherein CBM is

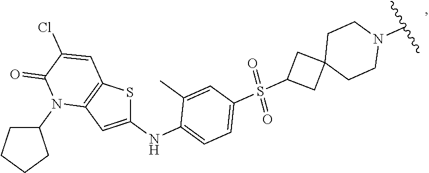 [SMILES: *N1CCC2(CC1)CC(S(=O)(=O)c1ccc(Nc3cc4c(cc(Cl)c(=O)n4C4CCCC4)s3)c(C)c1)C2]

LBM is selected from any of those in Table A below, and L is selected from any of those in Table B below.

In some embodiments, a provided compound or pharmaceutically acceptable salt thereof, is selected from those wherein CBM is

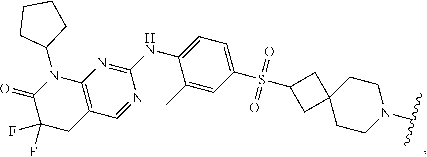 [SMILES: *N1CCC2(CC1)CC(S(=O)(=O)c1ccc(Nc3ncc4c(n3)N(C3CCCC3)C(=O)C(F)(F)C4)c(C)c1)C2]

LBM is selected from any of those in Table A below, and L is selected from any of those in Table B below.

In some embodiments, a provided compound or pharmaceutically acceptable salt thereof, is selected from those wherein CBM is

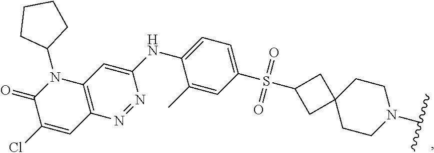 [SMILES: *N1CCC2(CC1)CC(S(=O)(=O)c1ccc(Nc3cc4c(cc(Cl)c(=O)n4C4CCCC4)nn3)c(C)c1)C2]

LBM is selected from any of those in Table A below, and L is selected from any of those in Table B below.

In some embodiments, a provided compound or pharmaceutically acceptable salt thereof, is selected from those wherein CBM is

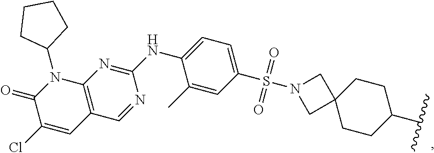 [SMILES: *C1CCC2(CC1)CN(S(=O)(=O)c1ccc(Nc3ncc4cc(Cl)c(=O)n(C5CCCC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

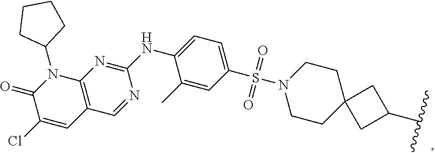 [SMILES: *C1CC2(CCN(S(=O)(=O)c3ccc(Nc4ncc5cc(Cl)c(=O)n(C6CCCC6)c5n4)c(C)c3)CC2)C1]

LBM is selected from any of those in Table A below, and L is selected from any of those in Table B below.

In some embodiments, a provided compound or pharmaceutically acceptable salt thereof, is selected from those wherein CBM is

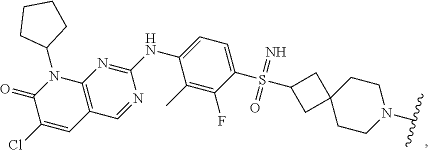 [SMILES: *N1CCC2(CC1)CC(S(=N)(=O)c1ccc(Nc3ncc4cc(Cl)c(=O)n(C5CCCC5)c4n3)c(C)c1F)C2]

LBM is selected from any of those in Table A below, and L is selected from any of those in Table B below.

In some embodiments, a provided compound or pharmaceutically acceptable salt thereof, is selected from those wherein CBM is

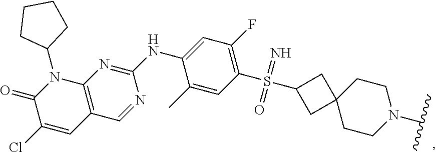 [SMILES: *N1CCC2(CC1)CC(S(=N)(=O)c1cc(C)c(Nc3ncc4cc(Cl)c(=O)n(C5CCCC5)c4n3)cc1F)C2]

LBM is selected from any of those in Table A below, and L is selected from any of those in Table B below.

In some embodiments, a provided compound or pharmaceutically acceptable salt thereof, is selected from those wherein CBM is

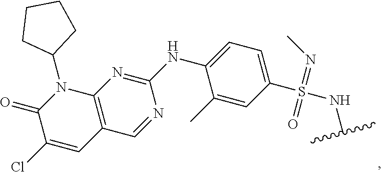 [SMILES: *NS(=O)(=NC)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

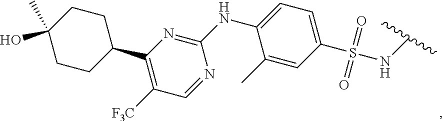 [SMILES: *NS(=O)(=O)c1ccc(Nc2ncc(C(F)(F)F)c([C@H]3CC[C@@](C)(O)CC3)n2)c(C)c1]

LBM is selected from any of those in Table A below, and L is selected from any of those in Table B below.

In some embodiments, a provided compound or pharmaceutically acceptable salt thereof, is selected from those wherein CBM is

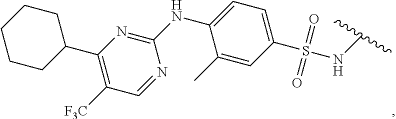 [SMILES: *NS(=O)(=O)c1ccc(Nc2ncc(C(F)(F)F)c(C3CCCCC3)n2)c(C)c1]

LBM is selected from any of those in Table A below, and L is selected from any of those in Table B below.

In some embodiments, a provided compound or pharmaceutically acceptable salt thereof, is selected from those wherein CBM is

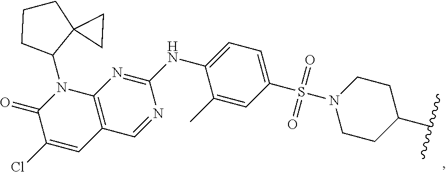 [SMILES: *C1CCN(S(=O)(=O)c2ccc(Nc3ncc4cc(Cl)c(=O)n(C5CCCC56CC6)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

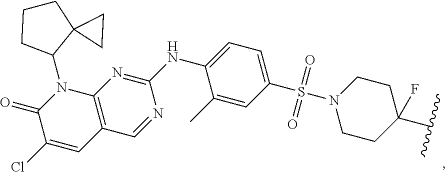 [SMILES: *C1(F)CCN(S(=O)(=O)c2ccc(Nc3ncc4cc(Cl)c(=O)n(C5CCCC56CC6)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

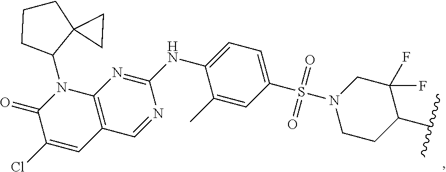 [SMILES: *C1CCN(S(=O)(=O)c2ccc(Nc3ncc4cc(Cl)c(=O)n(C5CCCC56CC6)c4n3)c(C)c2)CC1(F)F]

BM is selected from any of those in Table A below, and L is selected from any of those in Table B below.

In some embodiments, a provided compound or pharmaceutically acceptable salt thereof, is selected from those wherein CBM is

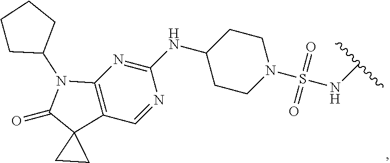 [SMILES: *NS(=O)(=O)N1CCC(Nc2ncc3c(n2)N(C2CCCC2)C(=O)C32CC2)CC1]

LBM is selected from any of those in Table A below, and L is selected from any of those in Table B below.

In some embodiments, a provided compound or pharmaceutically acceptable salt thereof, is selected from those wherein CBM is

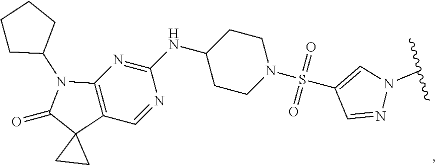 [SMILES: *n1cc(S(=O)(=O)N2CCC(Nc3ncc4c(n3)N(C3CCCC3)C(=O)C43CC3)CC2)cn1]

LBM is selected from any of those in Table A below, and L is selected from any of those in Table B below.

In some embodiments, a provided compound or pharmaceutically acceptable salt thereof, is selected from those wherein CBM is

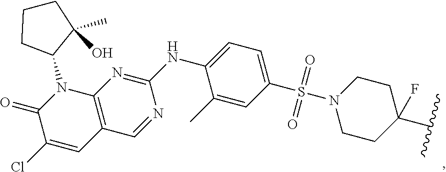 [SMILES: *C1(F)CCN(S(=O)(=O)c2ccc(Nc3ncc4cc(Cl)c(=O)n([C@@H]5CCC[C@@]5(C)O)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

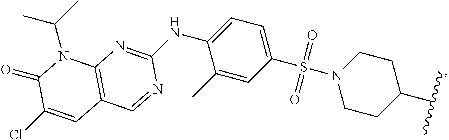 [SMILES: *C1CCN(S(=O)(=O)c2ccc(Nc3ncc4cc(Cl)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

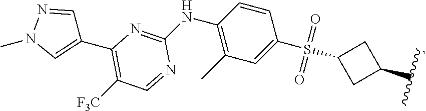 [SMILES: *[C@H]1C[C@H](S(=O)(=O)c2ccc(Nc3ncc(C(F)(F)F)c(-c4cnn(C)c4)n3)c(C)c2)C1]

LBM is selected from any of those in Table A below, and L is selected from any of those in Table B below.

In some embodiments, a provided compound or pharmaceutically acceptable salt thereof, is selected from those wherein CBM is

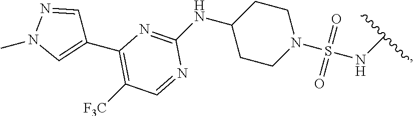 [SMILES: *NS(=O)(=O)N1CCC(Nc2ncc(C(F)(F)F)c(-c3cnn(C)c3)n2)CC1]

LBM is selected from any of those in Table A below, and L is selected from any of those in Table B below.

In some embodiments, a provided compound or pharmaceutically acceptable salt thereof, is selected from those wherein CBM is

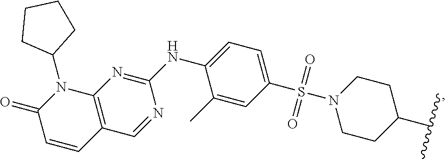 [SMILES: *C1CCN(S(=O)(=O)c2ccc(Nc3ncc4ccc(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

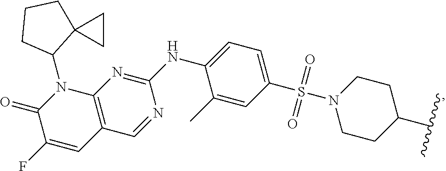 [SMILES: *C1CCN(S(=O)(=O)c2ccc(Nc3ncc4cc(F)c(=O)n(C5CCCC56CC6)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

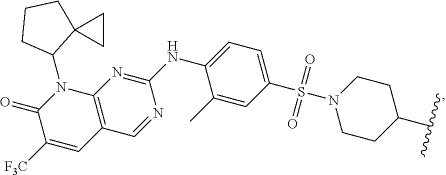 [SMILES: *C1CCN(S(=O)(=O)c2ccc(Nc3ncc4cc(C(F)(F)F)c(=O)n(C5CCCC56CC6)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

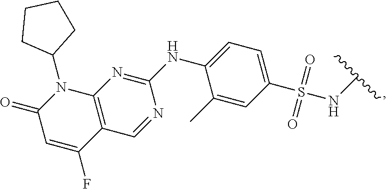 [SMILES: *NS(=O)(=O)c1ccc(Nc2ncc3c(F)cc(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

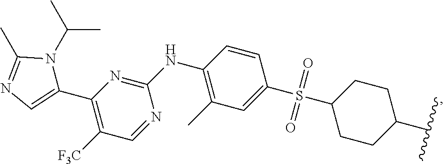 [SMILES: *C1CCC(S(=O)(=O)c2ccc(Nc3ncc(C(F)(F)F)c(-c4cnc(C)n4C(C)C)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

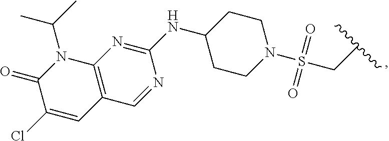 [SMILES: *CS(=O)(=O)N1CCC(Nc2ncc3cc(Cl)c(=O)n(C(C)C)c3n2)CC1]

LBM is selected from any of those in Table A below, and L is selected from any of those in Table B below.

In some embodiments, a provided compound or pharmaceutically acceptable salt thereof, is selected from those wherein CBM is

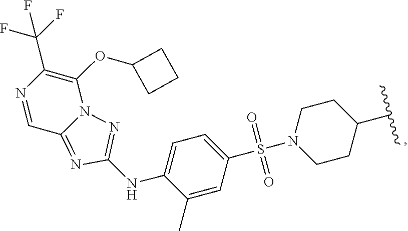 [SMILES: *C1CCN(S(=O)(=O)c2ccc(Nc3nc4cnc(C(F)(F)F)c(OC5CCC5)n4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

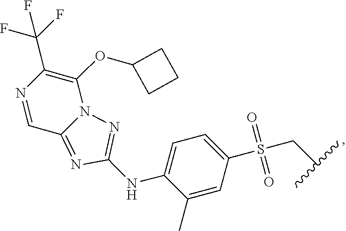 [SMILES: *CS(=O)(=O)c1ccc(Nc2nc3cnc(C(F)(F)F)c(OC4CCC4)n3n2)c(C)c1]

LBM is selected from any of those in Table A below, and L is selected from any of those in Table B below.

In some embodiments, a provided compound or pharmaceutically acceptable salt thereof, is selected from those wherein CBM is

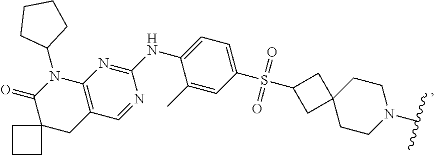 [SMILES: *N1CCC2(CC1)CC(S(=O)(=O)c1ccc(Nc3ncc4c(n3)N(C3CCCC3)C(=O)C3(CCC3)C4)c(C)c1)C2]

LBM is selected from any of those in Table A below, and L is selected from any of those in Table B below.

In some embodiments, a provided compound or pharmaceutically acceptable salt thereof, is selected from those wherein CBM is

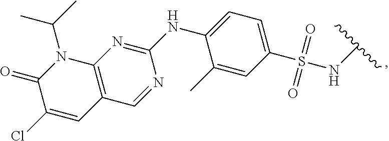 [SMILES: *NS(=O)(=O)c1ccc(Nc2ncc3cc(Cl)c(=O)n(C(C)C)c3n2)c(C)c1]

LBM is selected from any of those in Table A below, and L is selected from any of those in Table B below.

In some embodiments, a provided compound or pharmaceutically acceptable salt thereof, is selected from those wherein CBM is

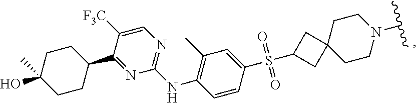 [SMILES: *N1CCC2(CC1)CC(S(=O)(=O)c1ccc(Nc3ncc(C(F)(F)F)c([C@H]4CC[C@@](C)(O)CC4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

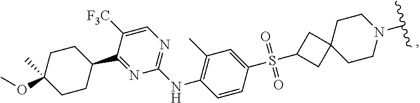 [SMILES: *N1CCC2(CC1)CC(S(=O)(=O)c1ccc(Nc3ncc(C(F)(F)F)c([C@H]4CC[C@@](C)(OC)CC4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

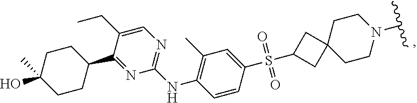 [SMILES: *N1CCC2(CC1)CC(S(=O)(=O)c1ccc(Nc3ncc(CC)c([C@H]4CC[C@@](C)(O)CC4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

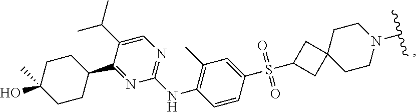 [SMILES: *N1CCC2(CC1)CC(S(=O)(=O)c1ccc(Nc3ncc(C(C)C)c([C@H]4CC[C@@](C)(O)CC4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

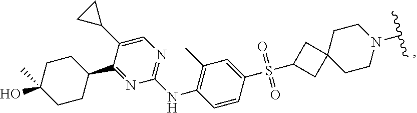 [SMILES: *N1CCC2(CC1)CC(S(=O)(=O)c1ccc(Nc3ncc(C4CC4)c([C@H]4CC[C@@](C)(O)CC4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

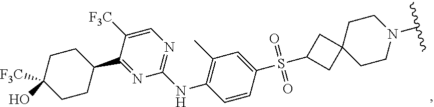

LBM is selected from any of those in Table A below, and L is selected from any of those in Table B below.

In some embodiments, a provided compound or pharmaceutically acceptable salt thereof, is selected from those wherein CBM is

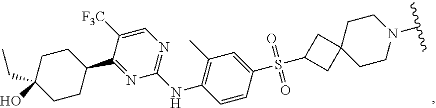

LBM is selected from any of those in Table A below, and L is selected from any of those in Table B below.

In some embodiments, a provided compound or pharmaceutically acceptable salt thereof, is selected from those wherein CBM is

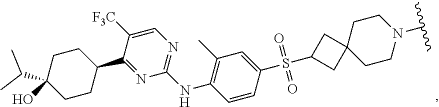

LBM is selected from any of those in Table A below, and L is selected from any of those in Table B below.

In some embodiments, a provided compound or pharmaceutically acceptable salt thereof, is selected from those wherein CBM is

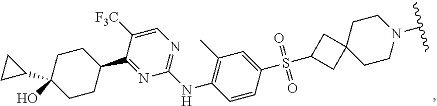

LBM is selected from any of those in Table A below, and L is selected from any of those in Table B below.

In some embodiments, a provided compound or pharmaceutically acceptable salt thereof, is selected from those wherein CBM is

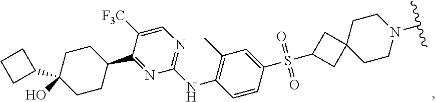

LBM is selected from any of those in Table A below, and L is selected from any of those in Table B below.

In some embodiments, a provided compound or pharmaceutically acceptable salt thereof, is selected from those wherein CBM is

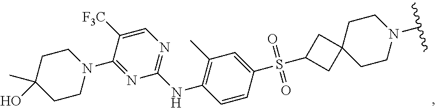

LBM is selected from any of those in Table A below, and L is selected from any of those in Table B below.

In some embodiments, a provided compound or pharmaceutically acceptable salt thereof, is selected from those wherein CBM is

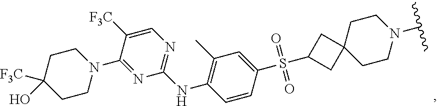

LBM is selected from any of those in Table A below, and L is selected from any of those in Table B below.

In some embodiments, a provided compound or pharmaceutically acceptable salt thereof, is selected from those wherein CBM is

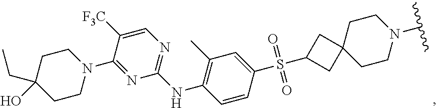

LBM is selected from any of those in Table A below, and L is selected from any of those in Table B below.

In some embodiments, a provided compound or pharmaceutically acceptable salt thereof, is selected from those wherein CBM is

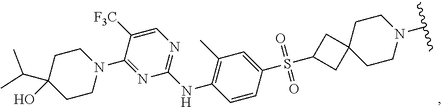

LBM is selected from any of those in Table A below, and L is selected from any of those in Table B below.

In some embodiments, a provided compound or pharmaceutically acceptable salt thereof, is selected from those wherein CBM is

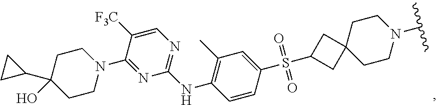

LBM is selected from any of those in Table A below, and L is selected from any of those in Table B below.

In some embodiments, a provided compound or pharmaceutically acceptable salt thereof, is selected from those wherein CBM is

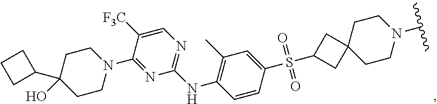

LBM is selected from any of those in Table A below, and L is selected from any of those in Table B below.

In some embodiments, a provided compound or pharmaceutically acceptable salt thereof, is selected from those wherein CBM is

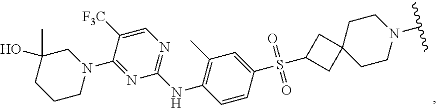

LBM is selected from any of those in Table A below, and L is selected from any of those in Table B below.

In some embodiments, a provided compound or pharmaceutically acceptable salt thereof, is selected from those wherein CBM is

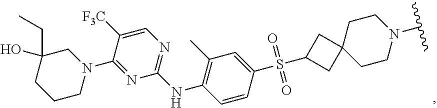

LBM is selected from any of those in Table A below, and L is selected from any of those in Table B below.

In some embodiments, a provided compound or pharmaceutically acceptable salt thereof, is selected from those wherein CBM is

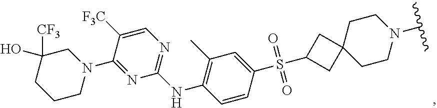

LBM is selected from any of those in Table A below, and L is selected from any of those in Table B below.

In some embodiments, a provided compound or pharmaceutically acceptable salt thereof, is selected from those wherein CBM is

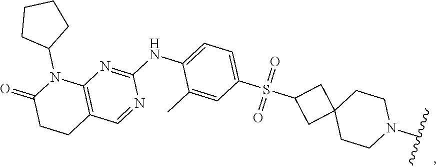

LBM is selected from any of those in Table A below, and L is selected from any of those in Table B below.

In some embodiments, a provided compound or pharmaceutically acceptable salt thereof, is selected from those wherein CBM is

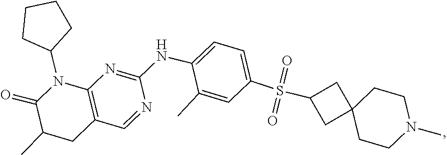 [SMILES: Cc1cc(S(=O)(=O)C2CC3(CCN(C)CC3)C2)ccc1Nc1ncc2c(n1)N(C1CCCC1)C(=O)C(C)C2]

LBM is selected from any of those in Table A below, and L is selected from any of those in Table B below.

In some embodiments, a provided compound or pharmaceutically acceptable salt thereof, is selected from those wherein CBM is

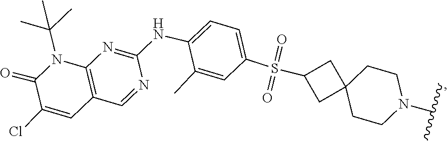 [SMILES: *N1CCC2(CC1)CC(S(=O)(=O)c1ccc(Nc3ncc4cc(Cl)c(=O)n(C(C)(C)C)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

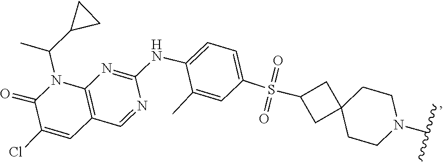 [SMILES: *N1CCC2(CC1)CC(S(=O)(=O)c1ccc(Nc3ncc4cc(Cl)c(=O)n(C(C)C5CC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

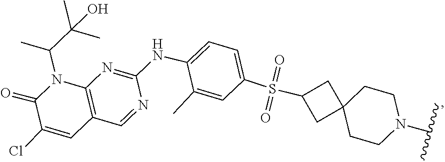 [SMILES: *N1CCC2(CC1)CC(S(=O)(=O)c1ccc(Nc3ncc4cc(Cl)c(=O)n(C(C)C(C)(C)O)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

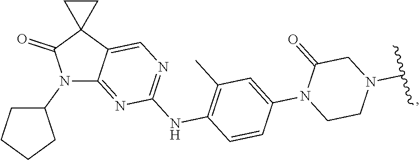 [SMILES: *N1CCN(c2ccc(Nc3ncc4c(n3)N(C3CCCC3)C(=O)C43CC3)c(C)c2)C(=O)C1]

LBM is selected from any of those in Table A below, and L is selected from any of those in Table B below.

In some embodiments, a provided compound or pharmaceutically acceptable salt thereof, is selected from those wherein CBM is

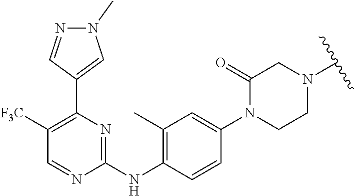 [SMILES: *N1CCN(c2ccc(Nc3ncc(C(F)(F)F)c(-c4cnn(C)c4)n3)c(C)c2)C(=O)C1]

LBM is selected from any of those in Table A below, and L is selected from any of those in Table B below.

In some embodiments, a provided compound or pharmaceutically acceptable salt thereof, is selected from those wherein CBM is

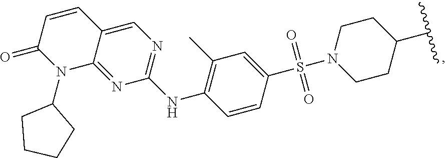 [SMILES: *C1CCN(S(=O)(=O)c2ccc(Nc3ncc4ccc(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

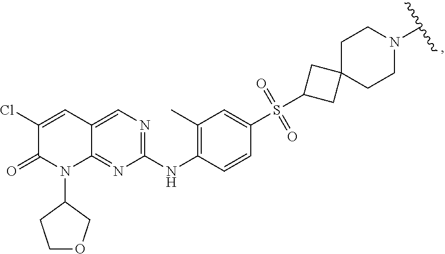 [SMILES: *N1CCC2(CC1)CC(S(=O)(=O)c1ccc(Nc3ncc4cc(Cl)c(=O)n(C5CCOC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

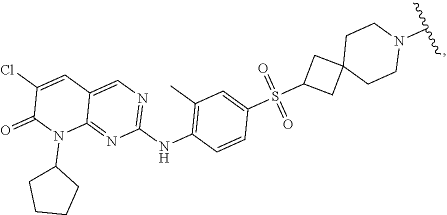 [SMILES: *N1CCC2(CC1)CC(S(=O)(=O)c1ccc(Nc3ncc4cc(Cl)c(=O)n(C5CCCC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

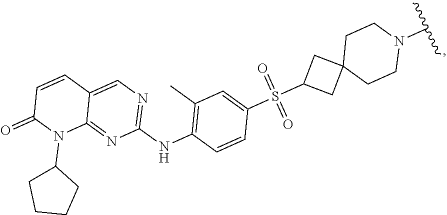 [SMILES: *N1CCC2(CC1)CC(S(=O)(=O)c1ccc(Nc3ncc4ccc(=O)n(C5CCCC5)c4n3)c(C)c1)C2]

LBM selected from any of those in Table A below, and L is selected from any of those in Table B below.

In some embodiments, a provided compound or pharmaceutically acceptable salt thereof, is selected from those wherein CBM is

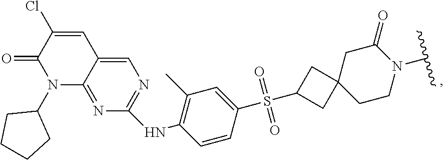 [SMILES: *N1CCC2(CC1=O)CC(S(=O)(=O)c1ccc(Nc3ncc4cc(Cl)c(=O)n(C5CCCC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

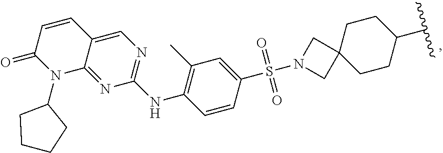 [SMILES: *C1CCC2(CC1)CN(S(=O)(=O)c1ccc(Nc3ncc4ccc(=O)n(C5CCCC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

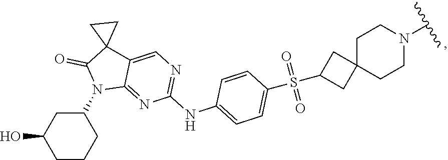

LBM is selected from any of those in Table A below, and L is selected from any of those in Table B below.

In some embodiments, a provided compound or pharmaceutically acceptable salt thereof, is selected from those wherein CBM is

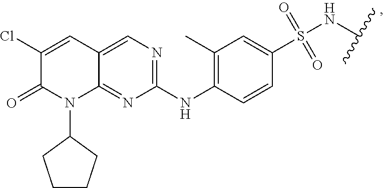 [SMILES: *NS(=O)(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

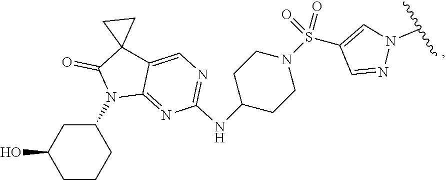 [SMILES: *n1cc(S(=O)(=O)N2CCC(Nc3ncc4c(n3)N([C@@H]3CCC[C@@H](O)C3)C(=O)C43CC3)CC2)cn1]

LBM is selected from any of those in Table A below, and L is selected from any of those in Table B below.

In some embodiments, a provided compound or pharmaceutically acceptable salt thereof, is selected from those wherein CBM is

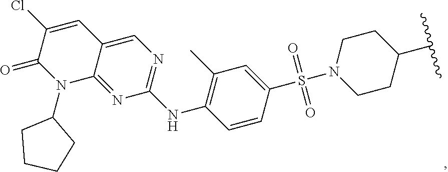 [SMILES: *C1CCN(S(=O)(=O)c2ccc(Nc3ncc4cc(Cl)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

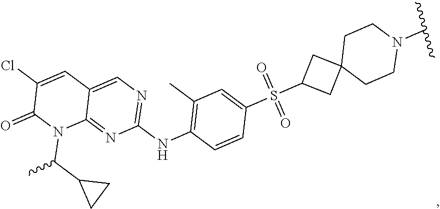 [SMILES: *N1CCC2(CC1)CC(S(=O)(=O)c1ccc(Nc3ncc4cc(Cl)c(=O)n(C(C)C5CC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

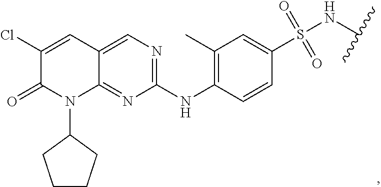 [SMILES: *NS(=O)(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

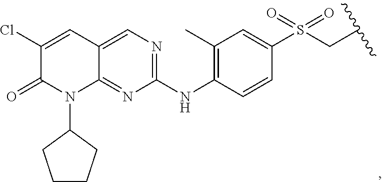 [SMILES: *CS(=O)(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

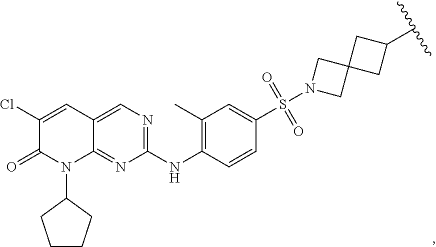 [SMILES: *C1CC2(C1)CN(S(=O)(=O)c1ccc(Nc3ncc4cc(Cl)c(=O)n(C5CCCC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

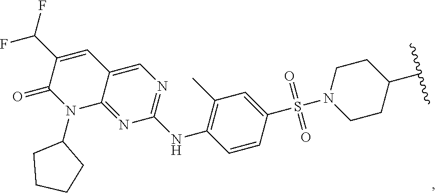 [SMILES: *C1CCN(S(=O)(=O)c2ccc(Nc3ncc4cc(C(F)F)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

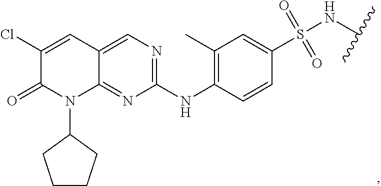 [SMILES: *NS(=O)(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

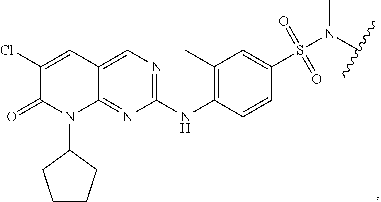 [SMILES: *N(C)S(=O)(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

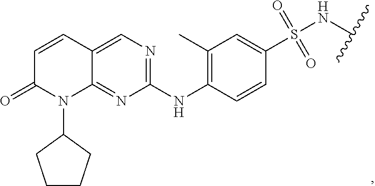 [SMILES: *NS(=O)(=O)c1ccc(Nc2ncc3ccc(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

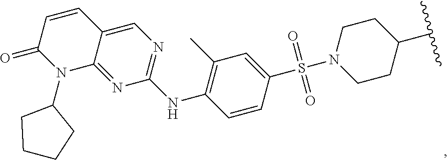 [SMILES: *C1CCN(S(=O)(=O)c2ccc(Nc3ncc4ccc(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

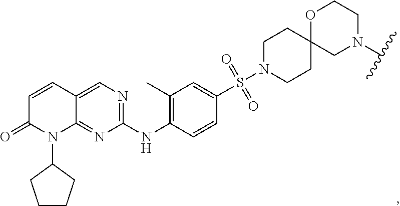 [SMILES: *N1CCOC2(CCN(S(=O)(=O)c3ccc(Nc4ncc5ccc(=O)n(C6CCCC6)c5n4)c(C)c3)CC2)C1]

LBM is selected from any of those in Table A below, and L is selected from any of those in Table B below.

In some embodiments, a provided compound or pharmaceutically acceptable salt thereof, is selected from those wherein CBM is

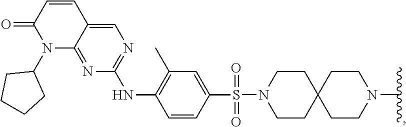 [SMILES: *N1CCC2(CC1)CCN(S(=O)(=O)c1ccc(Nc3ncc4ccc(=O)n(C5CCCC5)c4n3)c(C)c1)CC2]

LBM is selected from any of those in Table A below, and L is selected from any of those in Table B below.

In some embodiments, a provided compound or pharmaceutically acceptable salt thereof, is selected from those wherein CBM is

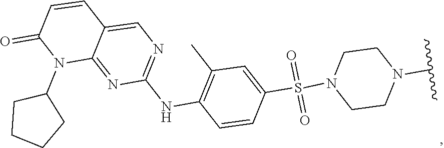 [SMILES: *N1CCN(S(=O)(=O)c2ccc(Nc3ncc4ccc(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

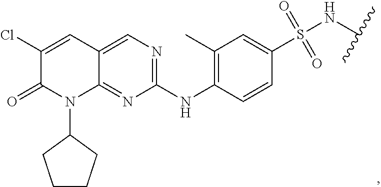 [SMILES: *NS(=O)(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

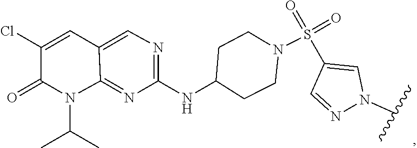 [SMILES: *n1cc(S(=O)(=O)N2CCC(Nc3ncc4cc(Cl)c(=O)n(C(C)C)c4n3)CC2)cn1]

LBM is selected from any of those in Table A below, and L is selected from any of those in Table B below.

In some embodiments, a provided compound or pharmaceutically acceptable salt thereof, is selected from those wherein CBM is

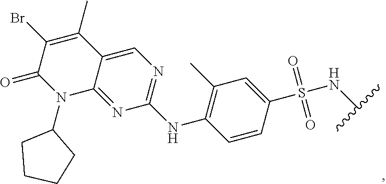 [SMILES: *NS(=O)(=O)c1ccc(Nc2ncc3c(C)c(Br)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

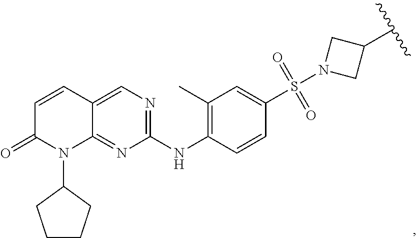 [SMILES: *C1CN(S(=O)(=O)c2ccc(Nc3ncc4ccc(=O)n(C5CCCC5)c4n3)c(C)c2)C1]

LBM is selected from any of those in Table A below, and L is selected from any of those in Table B below.

In some embodiments, a provided compound or pharmaceutically acceptable salt thereof, is selected from those wherein CBM is

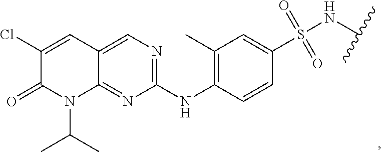 [SMILES: *NS(=O)(=O)c1ccc(Nc2ncc3cc(Cl)c(=O)n(C(C)C)c3n2)c(C)c1]

LBM is selected from any of those in Table A below, and L is selected from any of those in Table B below.

In some embodiments, a provided compound or pharmaceutically acceptable salt thereof, is selected from those wherein CBM is

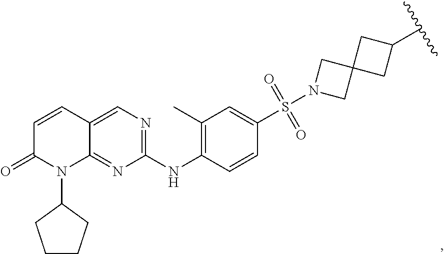 [SMILES: *C1CC2(C1)CN(S(=O)(=O)c1ccc(Nc3ncc4ccc(=O)n(C5CCCC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

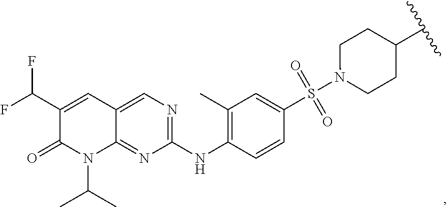 [SMILES: *C1CCN(S(=O)(=O)c2ccc(Nc3ncc4cc(C(F)F)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

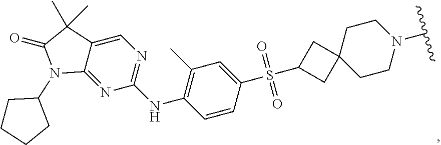 [SMILES: *N1CCC2(CC1)CC(S(=O)(=O)c1ccc(Nc3ncc4c(n3)N(C3CCCC3)C(=O)C4(C)C)c(C)c1)C2]

LBM is selected from any of those in Table A below, and L is selected from any of those in Table B below.

In some embodiments, a provided compound or pharmaceutically acceptable salt thereof, is selected from those wherein CBM is

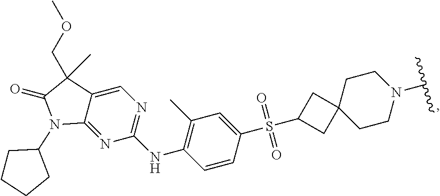 [SMILES: *N1CCC2(CC1)CC(S(=O)(=O)c1ccc(Nc3ncc4c(n3)N(C3CCCC3)C(=O)C4(C)COC)c(C)c1)C2]

LBM is selected from any of those in Table A below, and L is selected from any of those in Table B below.

In some embodiments, a provided compound or pharmaceutically acceptable salt thereof, is selected from those wherein CBM is

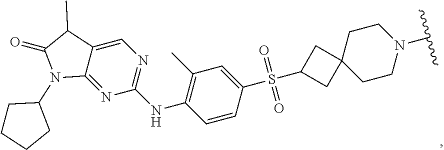 [SMILES: *N1CCC2(CC1)CC(S(=O)(=O)c1ccc(Nc3ncc4c(n3)N(C3CCCC3)C(=O)C4C)c(C)c1)C2]

LBM is selected from any of those in Table A below, and L is selected from any of those in Table B below.

In some embodiments, a provided compound or pharmaceutically acceptable salt thereof, is selected from those wherein CBM is

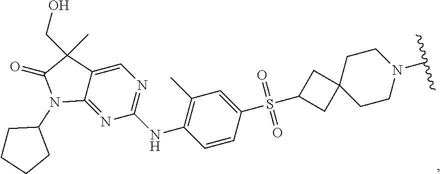 [SMILES: *N1CCC2(CC1)CC(S(=O)(=O)c1ccc(Nc3ncc4c(n3)N(C3CCCC3)C(=O)C4(C)CO)c(C)c1)C2]

LBM is selected from any of those in Table A below, and L is selected from any of those in Table B below.

In some embodiments, a provided compound or pharmaceutically acceptable salt thereof, is selected from those wherein CBM is

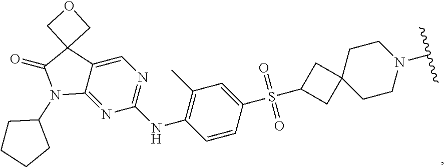 [SMILES: *N1CCC2(CC1)CC(S(=O)(=O)c1ccc(Nc3ncc4c(n3)N(C3CCCC3)C(=O)C43COC3)c(C)c1)C2]

LBM is selected from any of those in Table A below, and L is selected from any of those in Table B below.

In some embodiments, a provided compound or pharmaceutically acceptable salt thereof, is selected from those wherein CBM is

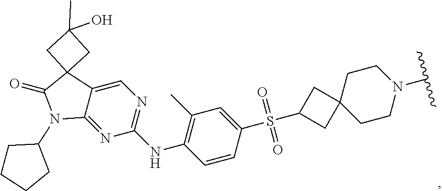 [SMILES: *N1CCC2(CC1)CC(S(=O)(=O)c1ccc(Nc3ncc4c(n3)N(C3CCCC3)C(=O)C43CC(C)(O)C3)c(C)c1)C2]

LBM is selected from any of those in Table A below, and L is selected from any of those in Table B below.

In some embodiments, a provided compound or pharmaceutically acceptable salt thereof, is selected from those wherein CBM is

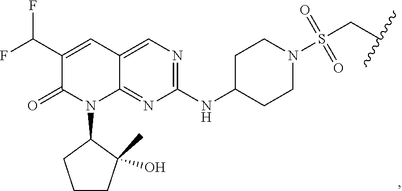 [SMILES: *CS(=O)(=O)N1CCC(Nc2ncc3cc(C(F)F)c(=O)n([C@@H]4CCC[C@@]4(C)O)c3n2)CC1]

LBM is selected from any of those in Table A below, and L is selected from any of those in Table B below.

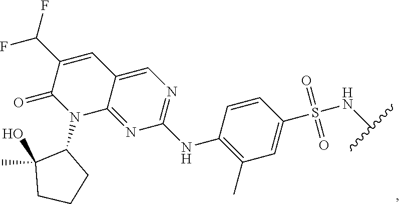 [SMILES: *NS(=O)(=O)c1ccc(Nc2ncc3cc(C(F)F)c(=O)n([C@@H]4CCC[C@@]4(C)O)c3n2)c(C)c1]

In some embodiments, a provided compound or pharmaceutically acceptable salt thereof, is selected from those wherein CBM is

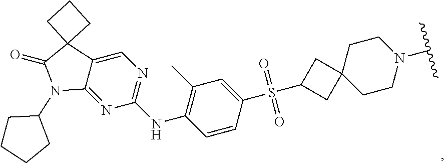 [SMILES: *N1CCC2(CC1)CC(S(=O)(=O)c1ccc(Nc3ncc4c(n3)N(C3CCCC3)C(=O)C43CCC3)c(C)c1)C2]

LBM is selected from any of those in Table A below, and L is selected from any of those in Table B below.

In some embodiments, a provided compound or pharmaceutically acceptable salt thereof, is selected from those wherein CBM is

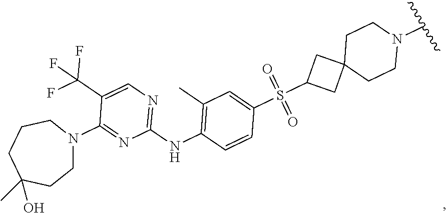 [SMILES: *N1CCC2(CC1)CC(S(=O)(=O)c1ccc(Nc3ncc(C(F)(F)F)c(N4CCCC(C)(O)CC4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

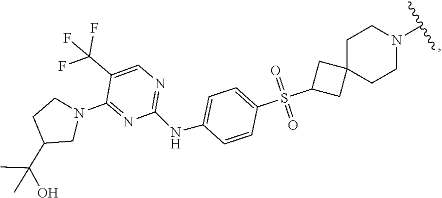 [SMILES: *N1CCC2(CC1)CC(S(=O)(=O)c1ccc(Nc3ncc(C(F)(F)F)c(N4CCC(C(C)(C)O)C4)n3)cc1)C2]

LBM is selected from any of those in Table A below, and L is selected from any of those in Table B below.

In some embodiments, a provided compound or pharmaceutically acceptable salt thereof, is selected from those wherein CBM is

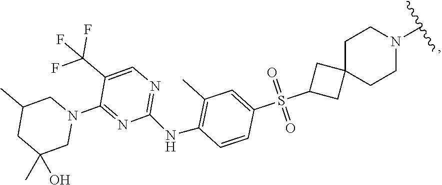 [SMILES: *N1CCC2(CC1)CC(S(=O)(=O)c1ccc(Nc3ncc(C(F)(F)F)c(N4CC(C)CC(C)(O)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

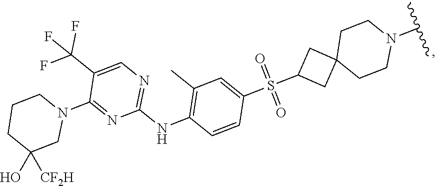 [SMILES: *N1CCC2(CC1)CC(S(=O)(=O)c1ccc(Nc3ncc(C(F)(F)F)c(N4CCCC(O)(C(F)F)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

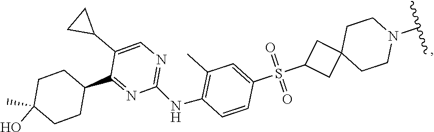 [SMILES: *N1CCC2(CC1)CC(S(=O)(=O)c1ccc(Nc3ncc(C4CC4)c([C@H]4CC[C@@](C)(O)CC4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

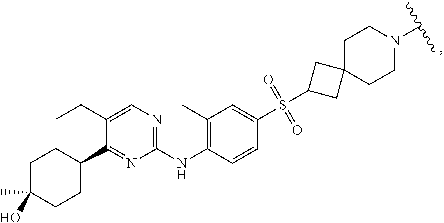 [SMILES: *N1CCC2(CC1)CC(S(=O)(=O)c1ccc(Nc3ncc(CC)c([C@H]4CC[C@@](C)(O)CC4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

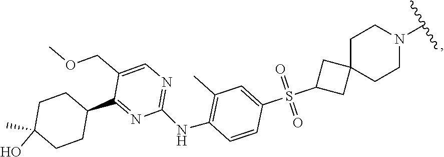 [SMILES: *N1CCC2(CC1)CC(S(=O)(=O)c1ccc(Nc3ncc(COC)c([C@H]4CC[C@@](C)(O)CC4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

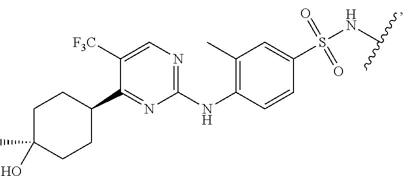 [SMILES: *NS(=O)(=O)c1ccc(Nc2ncc(C(F)(F)F)c([C@H]3CC[C@@](C)(O)CC3)n2)c(C)c1]

LBM is selected from any of those in Table A below, and L is selected from any of those in Table B below.

In some embodiments, a provided compound or pharmaceutically acceptable salt thereof, is selected from those wherein CBM is

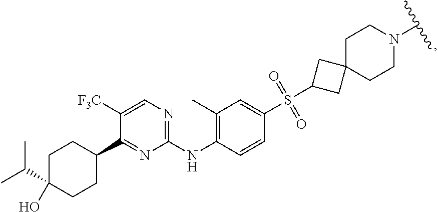 [SMILES: *N1CCC2(CC1)CC(S(=O)(=O)c1ccc(Nc3ncc(C(F)(F)F)c([C@H]4CC[C@](O)(C(C)C)CC4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

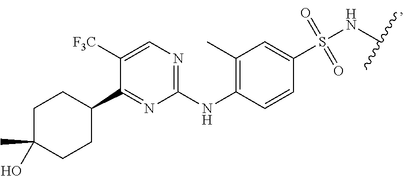 [SMILES: *NS(=O)(=O)c1ccc(Nc2ncc(C(F)(F)F)c([C@H]3CC[C@](C)(O)CC3)n2)c(C)c1]

LBM is selected from any of those in Table A below, and L is selected from any of those in Table B below.

In some embodiments, a provided compound or pharmaceutically acceptable salt thereof, is selected from those wherein CBM is

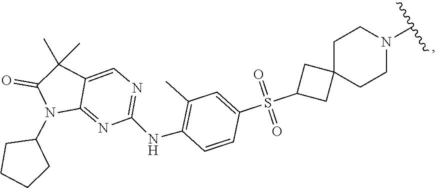 [SMILES: *N1CCC2(CC1)CC(S(=O)(=O)c1ccc(Nc3ncc4c(n3)N(C3CCCC3)C(=O)C4(C)C)c(C)c1)C2]

LBM is selected from any of those in Table A below, and L is selected from any of those in Table B below.

In some embodiments, a provided compound or pharmaceutically acceptable salt thereof, is selected from those wherein CBM is

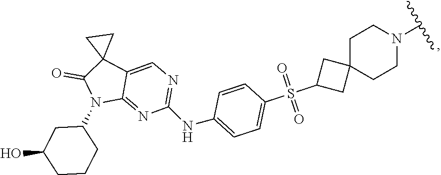

LBM is selected from any of those in Table A below, and L is selected from any of those in Table B below.

In some embodiments, a provided compound or pharmaceutically acceptable salt thereof, is selected from those wherein CBM is

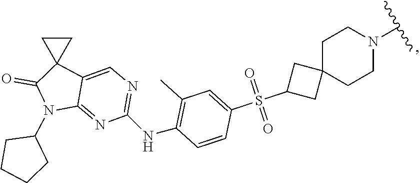 [SMILES: *N1CCC2(CC1)CC(S(=O)(=O)c1ccc(Nc3ncc4c(n3)N(C3CCCC3)C(=O)C43CC3)c(C)c1)C2]

LBM is selected from any of those in Table A below, and L is selected from any of those in Table B below.

In some embodiments, a provided compound or pharmaceutically acceptable salt thereof, is selected from those wherein CBM is

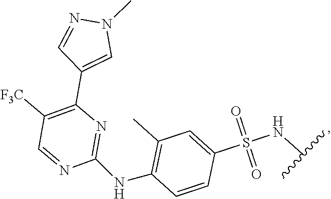 [SMILES: *NS(=O)(=O)c1ccc(Nc2ncc(C(F)(F)F)c(-c3cnn(C)c3)n2)c(C)c1]

LBM is selected from any of those in Table A below, and L is selected from any of those in Table B below.

In some embodiments, a provided compound or pharmaceutically acceptable salt thereof, is selected from those wherein CBM is

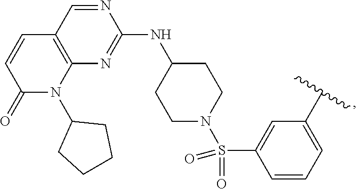 [SMILES: *c1cccc(S(=O)(=O)N2CCC(Nc3ncc4ccc(=O)n(C5CCCC5)c4n3)CC2)c1]

LBM is selected from any of those in Table A below, and L is selected from any of those in Table B below.

In some embodiments, a provided compound or pharmaceutically acceptable salt thereof, is selected from those wherein CBM is

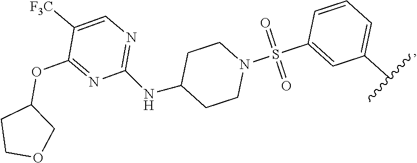 [SMILES: *c1cccc(S(=O)(=O)N2CCC(Nc3ncc(C(F)(F)F)c(OC4CCOC4)n3)CC2)c1]

LBM is selected from any of those in Table A below, and L is selected from any of those in Table B below.

In some embodiments, a provided compound or pharmaceutically acceptable salt thereof, is selected from those wherein CBM is

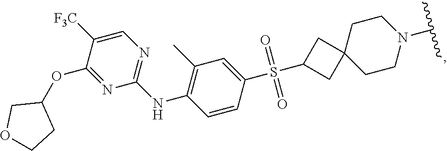 [SMILES: *N1CCC2(CC1)CC(S(=O)(=O)c1ccc(Nc3ncc(C(F)(F)F)c(OC4CCOC4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

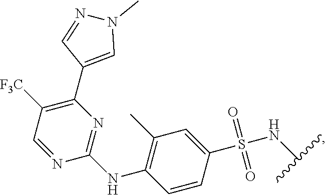 [SMILES: *NS(=O)(=O)c1ccc(Nc2ncc(C(F)(F)F)c(-c3cnn(C)c3)n2)c(C)c1]

LBM is selected from any of those in Table A below, and L is selected from any of those in Table B below.

In some embodiments, a provided compound or pharmaceutically acceptable salt thereof, is selected from those wherein CBM is

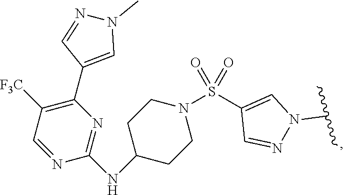 [SMILES: *n1cc(S(=O)(=O)N2CCC(Nc3ncc(C(F)(F)F)c(-c4cnn(C)c4)n3)CC2)cn1]

LBM is selected from any of those in Table A below, and L is selected from any of those in Table B below.

In some embodiments, a provided compound or pharmaceutically acceptable salt thereof, is selected from those wherein CBM is

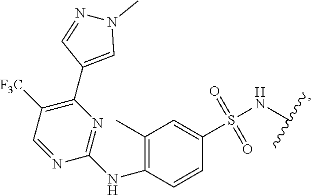 [SMILES: *NS(=O)(=O)c1ccc(Nc2ncc(C(F)(F)F)c(-c3cnn(C)c3)n2)c(C)c1]

LBM is selected from any of those in Table A below, and L is selected from any of those in Table B below.

In some embodiments, a provided compound or pharmaceutically acceptable salt thereof, is selected from those wherein CBM is

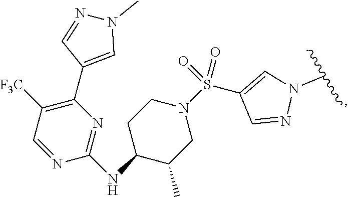 [SMILES: *n1cc(S(=O)(=O)N2CC[C@H](Nc3ncc(C(F)(F)F)c(-c4cnn(C)c4)n3)[C@@H](C)C2)cn1]

LBM is selected from any of those in Table A below, and L is selected from any of those in Table B below.

In some embodiments, a provided compound or pharmaceutically acceptable salt thereof, is selected from those wherein CBM is

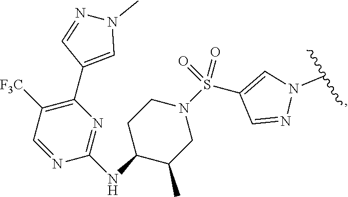 [SMILES: *n1cc(S(=O)(=O)N2CC[C@H](Nc3ncc(C(F)(F)F)c(-c4cnn(C)c4)n3)[C@H](C)C2)cn1]

LBM is selected from any of those in Table A below, and L is selected from any of those in Table B below.

In some embodiments, a provided compound or pharmaceutically acceptable salt thereof, is selected from those wherein CBM is

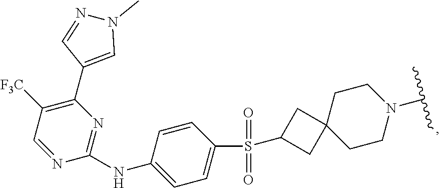 [SMILES: *N1CCC2(CC1)CC(S(=O)(=O)c1ccc(Nc3ncc(C(F)(F)F)c(-c4cnn(C)c4)n3)cc1)C2]

LBM is selected from any of those in Table A below, and L is selected from any of those in Table B below.

In some embodiments, a provided compound or pharmaceutically acceptable salt thereof, is selected from those wherein CBM is

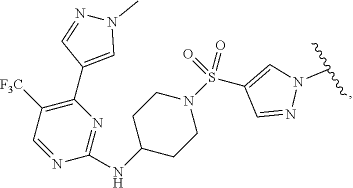 [SMILES: *n1cc(S(=O)(=O)N2CCC(Nc3ncc(C(F)(F)F)c(-c4cnn(C)c4)n3)CC2)cn1]

LBM is selected from any of those in Table A below, and L is selected from any of those in Table B below.

In some embodiments, a provided compound or pharmaceutically acceptable salt thereof, is selected from those wherein CBM is

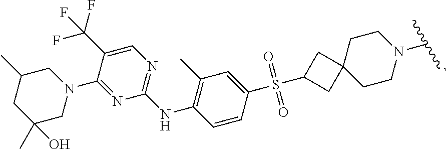 [SMILES: *N1CCC2(CC1)CC(S(=O)(=O)c1ccc(Nc3ncc(C(F)(F)F)c(N4CC(C)CC(C)(O)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

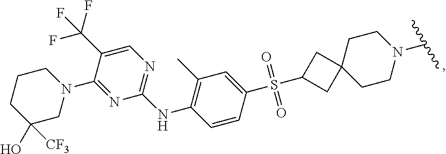 [SMILES: *N1CCC2(CC1)CC(S(=O)(=O)c1ccc(Nc3ncc(C(F)(F)F)c(N4CCCC(O)(C(F)(F)F)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

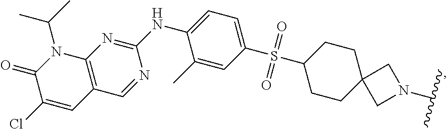 [SMILES: *N1CC2(CCC(S(=O)(=O)c3ccc(Nc4ncc5cc(Cl)c(=O)n(C(C)C)c5n4)c(C)c3)CC2)C1]

LBM is selected from any of those in Table A below, and L is selected from any of those in Table B below.

In some embodiments, a provided compound or pharmaceutically acceptable salt thereof, is selected from those wherein CBM is

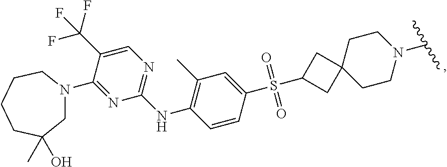 [SMILES: *N1CCC2(CC1)CC(S(=O)(=O)c1ccc(Nc3ncc(C(F)(F)F)c(N4CCCCC(C)(O)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

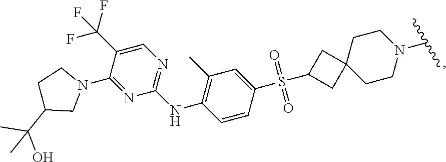 [SMILES: *N1CCC2(CC1)CC(S(=O)(=O)c1ccc(Nc3ncc(C(F)(F)F)c(N4CCC(C(C)(C)O)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

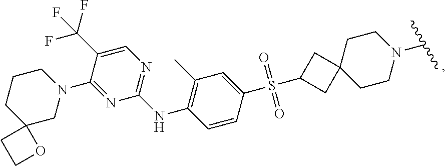 [SMILES: *N1CCC2(CC1)CC(S(=O)(=O)c1ccc(Nc3ncc(C(F)(F)F)c(N4CCCC5(CCO5)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

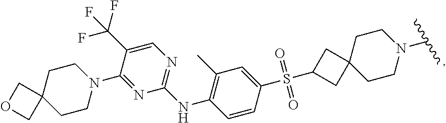 [SMILES: *N1CCC2(CC1)CC(S(=O)(=O)c1ccc(Nc3ncc(C(F)(F)F)c(N4CCC5(CC4)COC5)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

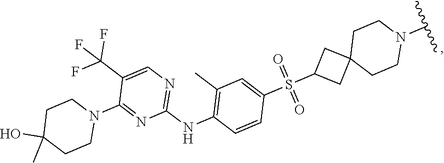 [SMILES: *N1CCC2(CC1)CC(S(=O)(=O)c1ccc(Nc3ncc(C(F)(F)F)c(N4CCC(C)(O)CC4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

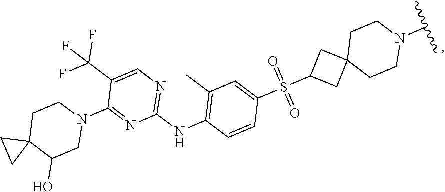 [SMILES: *N1CCC2(CC1)CC(S(=O)(=O)c1ccc(Nc3ncc(C(F)(F)F)c(N4CCC5(CC5)C(O)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

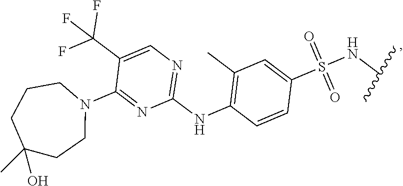 [SMILES: *NS(=O)(=O)c1ccc(Nc2ncc(C(F)(F)F)c(N3CCCC(C)(O)CC3)n2)c(C)c1]

LBM is selected from any of those in Table A below, and L is selected from any of those in Table B below.

In some embodiments, a provided compound or pharmaceutically acceptable salt thereof, is selected from those wherein CBM is

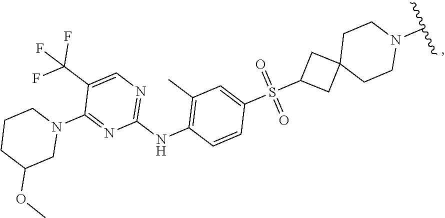 [SMILES: *N1CCC2(CC1)CC(S(=O)(=O)c1ccc(Nc3ncc(C(F)(F)F)c(N4CCCC(OC)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

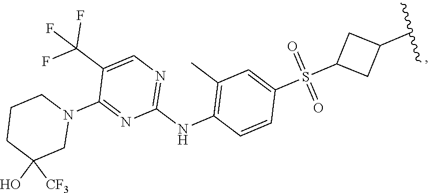 [SMILES: *C1CC(S(=O)(=O)c2ccc(Nc3ncc(C(F)(F)F)c(N4CCCC(O)(C(F)(F)F)C4)n3)c(C)c2)C1]

LBM is selected from any of those in Table A below, and L is selected from any of those in Table B below.

In some embodiments, a provided compound or pharmaceutically acceptable salt thereof, is selected from those wherein CBM is

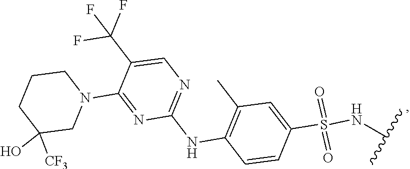 [SMILES: *NS(=O)(=O)c1ccc(Nc2ncc(C(F)(F)F)c(N3CCCC(O)(C(F)(F)F)C3)n2)c(C)c1]

LBM is selected from any of those in Table A below, and L is selected from any of those in Table B below.

In some embodiments, a provided compound or pharmaceutically acceptable salt thereof, is selected from those wherein CBM is

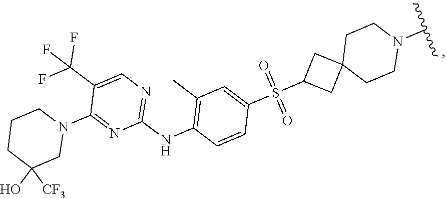 [SMILES: *N1CCC2(CC1)CC(S(=O)(=O)c1ccc(Nc3ncc(C(F)(F)F)c(N4CCCC(O)(C(F)(F)F)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

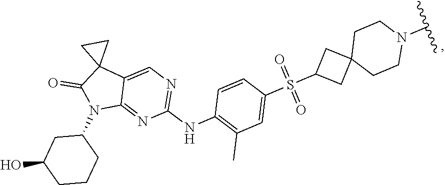 [SMILES: *N1CCC2(CC1)CC(S(=O)(=O)c1ccc(Nc3ncc4c(n3)N([C@@H]3CCC[C@@H](O)C3)C(=O)C43CC3)c(C)c1)C2]

LBM is selected from any of those in Table A below, and L is selected from any of those in Table B below.

In some embodiments, a provided compound or pharmaceutically acceptable salt thereof, is selected from those wherein CBM is

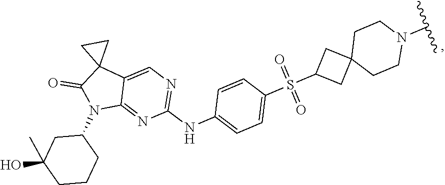 [SMILES: *N1CCC2(CC1)CC(S(=O)(=O)c1ccc(Nc3ncc4c(n3)N([C@@H]3CCC[C@@](C)(O)C3)C(=O)C43CC3)cc1)C2]

LBM is selected from any of those in Table A below, and L is selected from any of those in Table B below.

In some embodiments, a provided compound or pharmaceutically acceptable salt thereof, is selected from those wherein CBM is

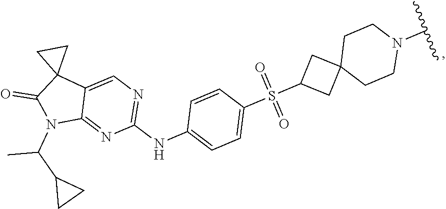 [SMILES: *N1CCC2(CC1)CC(S(=O)(=O)c1ccc(Nc3ncc4c(n3)N(C(C)C3CC3)C(=O)C43CC3)cc1)C2]

LBM is selected from any of those in Table A below, and L is selected from any of those in Table B below.

In some embodiments, a provided compound or pharmaceutically acceptable salt thereof, is selected from those wherein CBM is

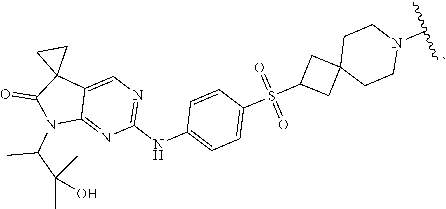 [SMILES: *N1CCC2(CC1)CC(S(=O)(=O)c1ccc(Nc3ncc4c(n3)N(C(C)C(C)(C)O)C(=O)C43CC3)cc1)C2]

LBM is selected from any of those in Table A below, and L is selected from any of those in Table B below.

In some embodiments, a provided compound or pharmaceutically acceptable salt thereof, is selected from those wherein CBM is

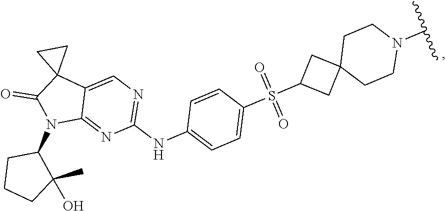 [SMILES: *N1CCC2(CC1)CC(S(=O)(=O)c1ccc(Nc3ncc4c(n3)N([C@@H]3CCC[C@@]3(C)O)C(=O)C43CC3)cc1)C2]

LBM is selected from any of those in Table A below, and L is selected from any of those in Table B below.

In some embodiments, a provided compound or pharmaceutically acceptable salt thereof, is selected from those wherein CBM is

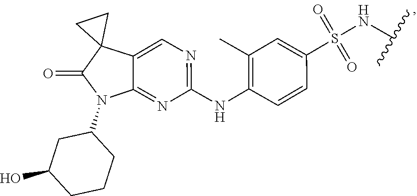

LBM is selected from any of those in Table A below, and L is selected from any of those in Table B below.

In some embodiments, a provided compound or pharmaceutically acceptable salt thereof, is selected from those wherein CBM is

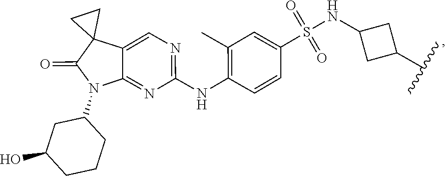

LBM is selected from any of those in Table A below, and L is selected from any of those in Table B below.

In some embodiments, a provided compound or pharmaceutically acceptable salt thereof, is selected from those wherein CBM is

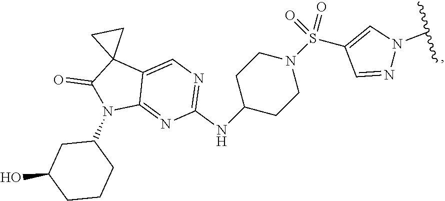

LBM is selected from any of those in Table A below, and L is selected from any of those in Table B below.

In some embodiments, a provided compound or pharmaceutically acceptable salt thereof, is selected from those wherein CBM is

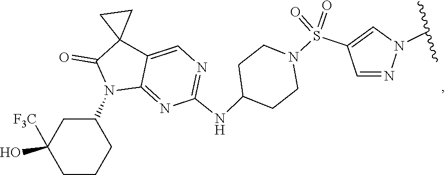

LBM is selected from any of those in Table A below, and L is selected from any of those in Table B below.

In some embodiments, a provided compound or pharmaceutically acceptable salt thereof, is selected from those wherein CBM is

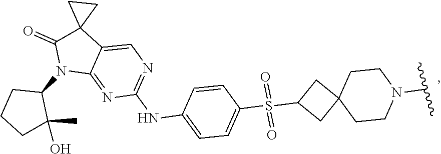 [SMILES: *N1CCC2(CC1)CC(S(=O)(=O)c1ccc(Nc3ncc4c(n3)N([C@@H]3CCC[C@@]3(C)O)C(=O)C43CC3)cc1)C2]

LBM is selected from any of those in Table A below, and L is selected from any of those in Table B below.

In some embodiments, a provided compound or pharmaceutically acceptable salt thereof, is selected from those wherein CBM is

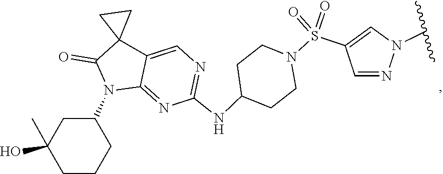

LBM is selected from any of those in Table A below, and L is selected from any of those in Table B below.

In some embodiments, a provided compound or pharmaceutically acceptable salt thereof, is selected from those wherein CBM is

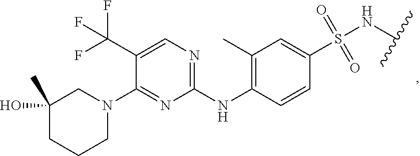 [SMILES: *NS(=O)(=O)c1ccc(Nc2ncc(C(F)(F)F)c(N3CCC[C@](C)(O)C3)n2)c(C)c1]

LBM is selected from any of those in Table A below, and L is selected from any of those in Table B below.

In some embodiments, a provided compound or pharmaceutically acceptable salt thereof, is selected from those wherein CBM is

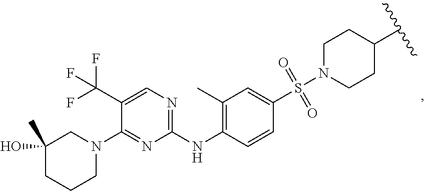 [SMILES: *C1CCN(S(=O)(=O)c2ccc(Nc3ncc(C(F)(F)F)c(N4CCC[C@](C)(O)C4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

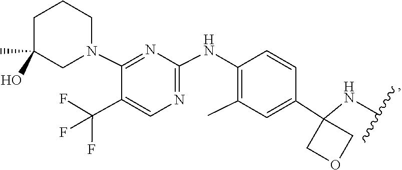 [SMILES: *NC1(c2ccc(Nc3ncc(C(F)(F)F)c(N4CCC[C@](C)(O)C4)n3)c(C)c2)COC1]

LBM is selected from any of those in Table A below, and L is selected from any of those in Table B below.

In some embodiments, a provided compound or pharmaceutically acceptable salt thereof, is selected from those wherein CBM is

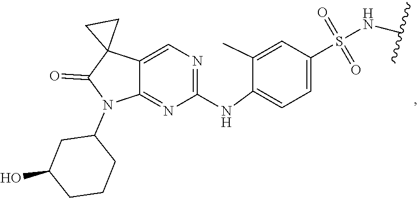 [SMILES: *NS(=O)(=O)c1ccc(Nc2ncc3c(n2)N(C2CCC[C@@H](O)C2)C(=O)C32CC2)c(C)c1]

LBM is selected from any of those in Table A below, and L is selected from any of those in Table B below.

In some embodiments, a provided compound or pharmaceutically acceptable salt thereof, is selected from those wherein CBM is

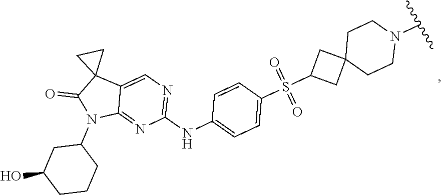 [SMILES: *N1CCC2(CC1)CC(S(=O)(=O)c1ccc(Nc3ncc4c(n3)N(C3CCC[C@@H](O)C3)C(=O)C43CC3)cc1)C2]

LBM is selected from any of those in Table A below, and L is selected from any of those in Table B below.

In some embodiments, a provided compound or pharmaceutically acceptable salt thereof, is selected from those wherein CBM is

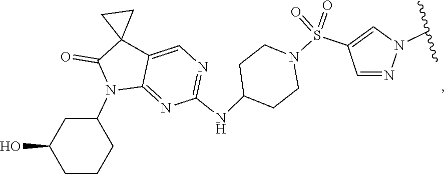 [SMILES: *n1cc(S(=O)(=O)N2CCC(Nc3ncc4c(n3)N(C3CCC[C@@H](O)C3)C(=O)C43CC3)CC2)cn1]

LBM is selected from any of those in Table A below, and L is selected from any of those in Table B below.

In some embodiments, a provided compound or pharmaceutically acceptable salt thereof, is selected from those wherein CBM is

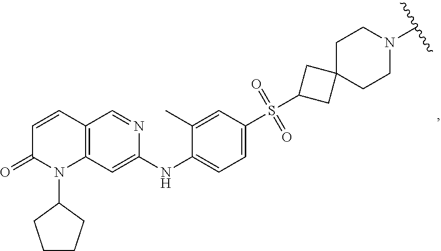 [SMILES: *N1CCC2(CC1)CC(S(=O)(=O)c1ccc(Nc3cc4c(ccc(=O)n4C4CCCC4)cn3)c(C)c1)C2]

LBM is selected from any of those in Table A below, and L is selected from any of those in Table B below.

In some embodiments, a provided compound or pharmaceutically acceptable salt thereof, is selected from those wherein CBM is

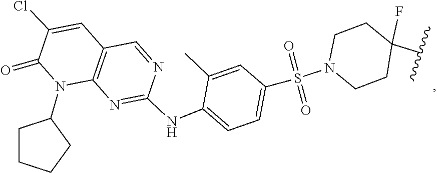 [SMILES: *C1(F)CCN(S(=O)(=O)c2ccc(Nc3ncc4cc(Cl)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

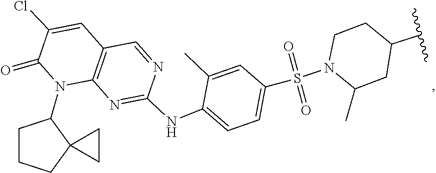 [SMILES: *C1CCN(S(=O)(=O)c2ccc(Nc3ncc4cc(Cl)c(=O)n(C5CCCC56CC6)c4n3)c(C)c2)C(C)C1]

LBM is selected from any of those in Table A below, and L is selected from any of those in Table B below.

In some embodiments, a provided compound or pharmaceutically acceptable salt thereof, is selected from those wherein CBM is

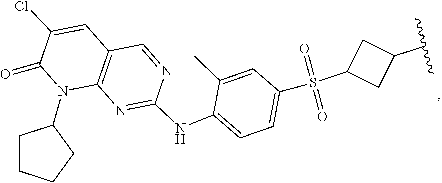 [SMILES: *C1CC(S(=O)(=O)c2ccc(Nc3ncc4cc(Cl)c(=O)n(C5CCCC5)c4n3)c(C)c2)C1]

LBM is selected from any of those in Table A below, and L is selected from any of those in Table B below.

In some embodiments, a provided compound or pharmaceutically acceptable salt thereof, is selected from those wherein CBM is

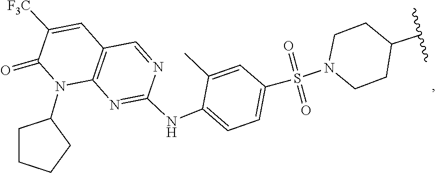 [SMILES: *C1CCN(S(=O)(=O)c2ccc(Nc3ncc4cc(C(F)(F)F)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

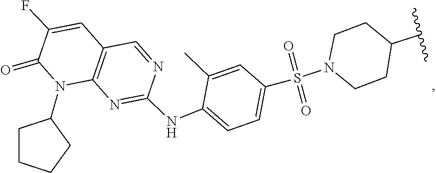 [SMILES: *C1CCN(S(=O)(=O)c2ccc(Nc3ncc4cc(F)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

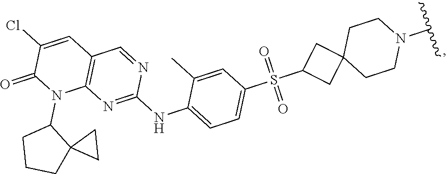 [SMILES: *N1CCC2(CC1)CC(S(=O)(=O)c1ccc(Nc3ncc4cc(Cl)c(=O)n(C5CCCC56CC6)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

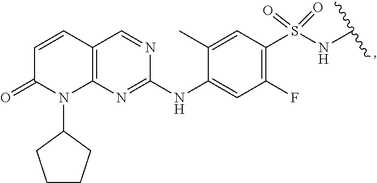 [SMILES: *NS(=O)(=O)c1cc(C)c(Nc2ncc3ccc(=O)n(C4CCCC4)c3n2)cc1F]

LBM is selected from any of those in Table A below, and L is selected from any of those in Table B below.

In some embodiments, a provided compound or pharmaceutically acceptable salt thereof, is selected from those wherein CBM is

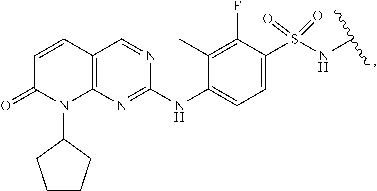 [SMILES: *NS(=O)(=O)c1ccc(Nc2ncc3ccc(=O)n(C4CCCC4)c3n2)c(C)c1F]

LBM is selected from any of those in Table A below, and L is selected from any of those in Table B below.

In some embodiments, a provided compound or pharmaceutically acceptable salt thereof, is selected from those wherein CBM is

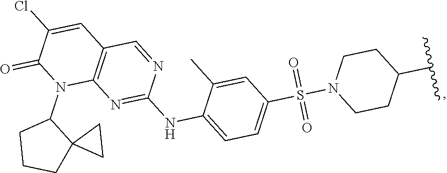 [SMILES: *C1CCN(S(=O)(=O)c2ccc(Nc3ncc4cc(Cl)c(=O)n(C5CCCC56CC6)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

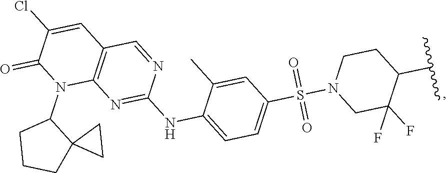 [SMILES: *C1CCN(S(=O)(=O)c2ccc(Nc3ncc4cc(Cl)c(=O)n(C5CCCC56CC6)c4n3)c(C)c2)CC1(F)F]

LBM is selected from any of those in Table A below, and L is selected from any of those in Table B below.

In some embodiments, a provided compound or pharmaceutically acceptable salt thereof, is selected from those wherein CBM is

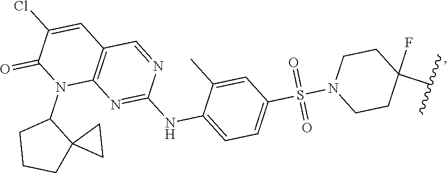 [SMILES: *C1(F)CCN(S(=O)(=O)c2ccc(Nc3ncc4cc(Cl)c(=O)n(C5CCCC56CC6)c4n3)c(C)c2)CC1]

LBM is selected from any of those in Table A below, and L is selected from any of those in Table B below.

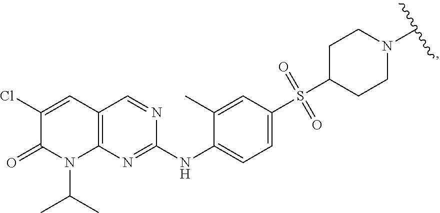 [SMILES: *N1CCC(S(=O)(=O)c2ccc(Nc3ncc4cc(Cl)c(=O)n(C(C)C)c4n3)c(C)c2)CC1]

In some embodiments, a provided compound or pharmaceutically acceptable salt thereof, is selected from those wherein CBM is

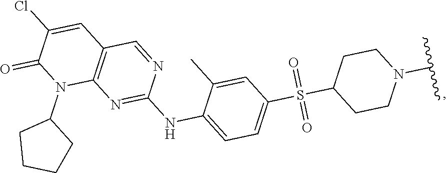 [SMILES: *N1CCC(S(=O)(=O)c2ccc(Nc3ncc4cc(Cl)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

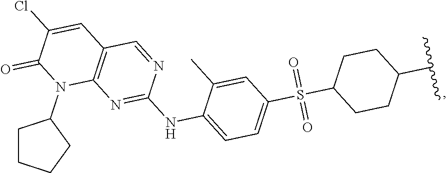 [SMILES: *C1CCC(S(=O)(=O)c2ccc(Nc3ncc4cc(Cl)c(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

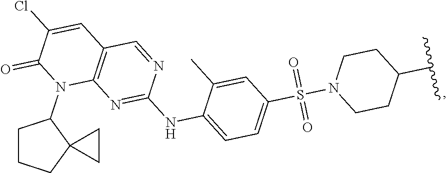 [SMILES: *C1CCN(S(=O)(=O)c2ccc(Nc3ncc4cc(Cl)c(=O)n(C5CCCC56CC6)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

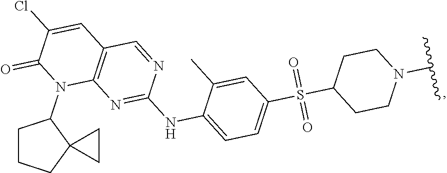 [SMILES: *N1CCC(S(=O)(=O)c2ccc(Nc3ncc4cc(Cl)c(=O)n(C5CCCC56CC6)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

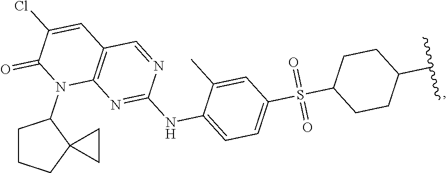 [SMILES: *C1CCC(S(=O)(=O)c2ccc(Nc3ncc4cc(Cl)c(=O)n(C5CCCC56CC6)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

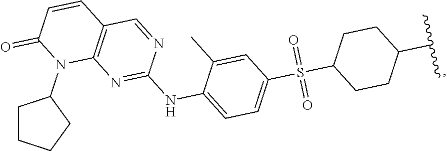 [SMILES: *C1CCC(S(=O)(=O)c2ccc(Nc3ncc4ccc(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

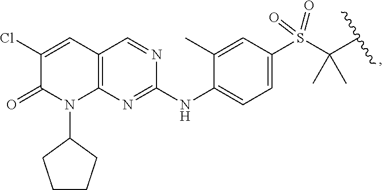 [SMILES: *C(C)(C)S(=O)(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

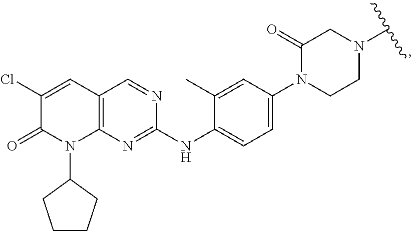 [SMILES: *N1CCN(c2ccc(Nc3ncc4cc(Cl)c(=O)n(C5CCCC5)c4n3)c(C)c2)C(=O)C1]

LBM is selected from any of those in Table A below, and L is selected from any of those in Table B below.

In some embodiments, a provided compound or pharmaceutically acceptable salt thereof, is selected from those wherein CBM is

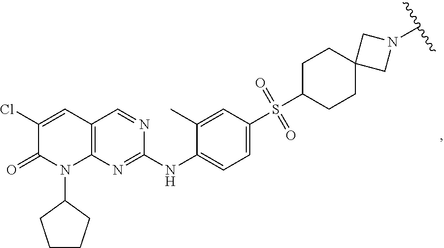 [SMILES: *N1CC2(CCC(S(=O)(=O)c3ccc(Nc4ncc5cc(Cl)c(=O)n(C6CCCC6)c5n4)c(C)c3)CC2)C1]

LBM is selected from any of those in Table A below, and L is selected from any of those in Table B below.

In some embodiments, a provided compound or pharmaceutically acceptable salt thereof, is selected from those wherein CBM is

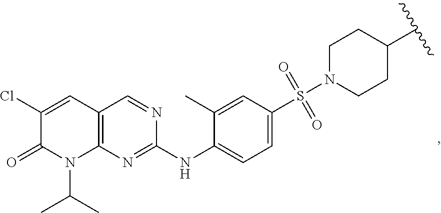 [SMILES: *C1CCN(S(=O)(=O)c2ccc(Nc3ncc4cc(Cl)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

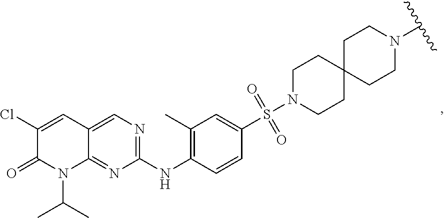 [SMILES: *N1CCC2(CC1)CCN(S(=O)(=O)c1ccc(Nc3ncc4cc(Cl)c(=O)n(C(C)C)c4n3)c(C)c1)CC2]

LBM is selected from any of those in Table A below, and L is selected from any of those in Table B below.

In some embodiments, a provided compound or pharmaceutically acceptable salt thereof, is selected from those wherein CBM is

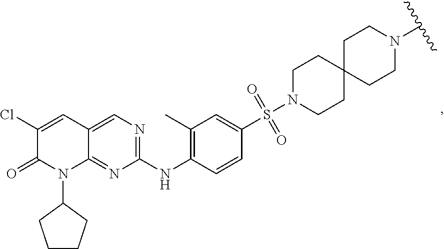 [SMILES: *N1CCC2(CC1)CCN(S(=O)(=O)c1ccc(Nc3ncc4cc(Cl)c(=O)n(C5CCCC5)c4n3)c(C)c1)CC2]

LBM is selected from any of those in Table A below, and L is selected from any of those in Table B below.

In some embodiments, a provided compound or pharmaceutically acceptable salt thereof, is selected from those wherein CBM is

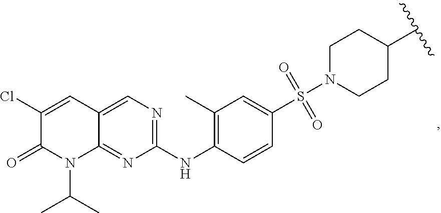 [SMILES: *C1CCN(S(=O)(=O)c2ccc(Nc3ncc4cc(Cl)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

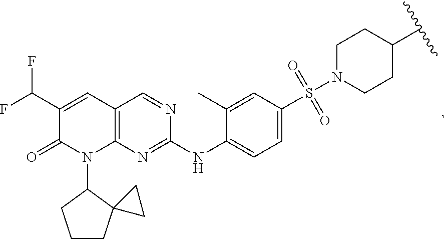 [SMILES: *C1CCN(S(=O)(=O)c2ccc(Nc3ncc4cc(C(F)F)c(=O)n(C5CCCC56CC6)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

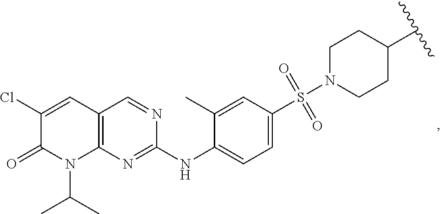 [SMILES: *C1CCN(S(=O)(=O)c2ccc(Nc3ncc4cc(Cl)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

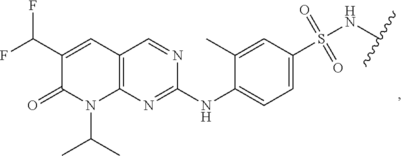 [SMILES: *NS(=O)(=O)c1ccc(Nc2ncc3cc(C(F)F)c(=O)n(C(C)C)c3n2)c(C)c1]

LBM is selected from any of those in Table A below, and L is selected from any of those in Table B below.

In some embodiments, a provided compound or pharmaceutically acceptable salt thereof, is selected from those wherein CBM is

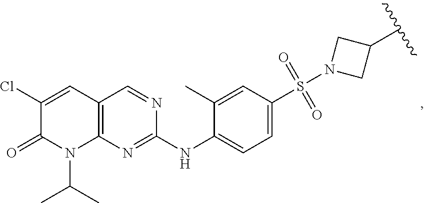 [SMILES: *C1CN(S(=O)(=O)c2ccc(Nc3ncc4cc(Cl)c(=O)n(C(C)C)c4n3)c(C)c2)C1]

LBM is selected from any of those in Table A below, and L is selected from any of those in Table B below.

In some embodiments, a provided compound or pharmaceutically acceptable salt thereof, is selected from those wherein CBM is

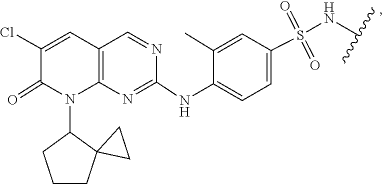 [SMILES: *NS(=O)(=O)c1ccc(Nc2ncc3cc(Cl)c(=O)n(C4CCCC45CC5)c3n2)c(C)c1]

LBM is selected from any of those in Table A below, and L is selected from any of those in Table B below.

In some embodiments, a provided compound or pharmaceutically acceptable salt thereof, is selected from those wherein CBM is

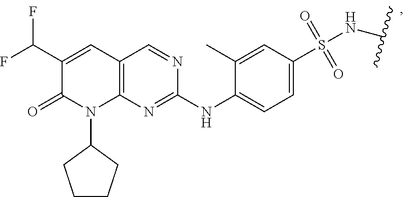 [SMILES: *NS(=O)(=O)c1ccc(Nc2ncc3cc(C(F)F)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

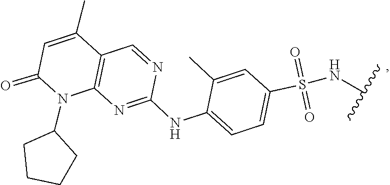 [SMILES: *NS(=O)(=O)c1ccc(Nc2ncc3c(C)cc(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

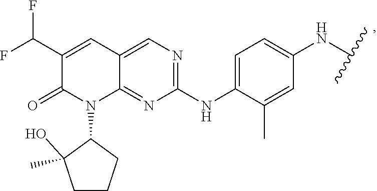 [SMILES: *Nc1ccc(Nc2ncc3cc(C(F)F)c(=O)n([C@@H]4CCC[C@@]4(C)O)c3n2)c(C)c1]

LBM is selected from any of those in Table A below, and L is selected from any of those in Table B below.

In some embodiments, a provided compound or pharmaceutically acceptable salt thereof, is selected from those wherein CBM is

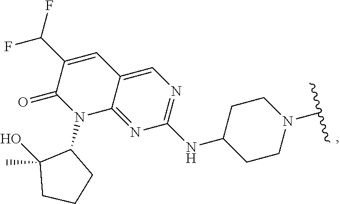 [SMILES: *N1CCC(Nc2ncc3cc(C(F)F)c(=O)n([C@@H]4CCC[C@@]4(C)O)c3n2)CC1]

LBM is selected from any of those in Table A below, and L is selected from any of those in Table B below.

In some embodiments, a provided compound or pharmaceutically acceptable salt thereof, is selected from those wherein CBM is

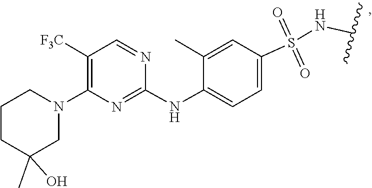 [SMILES: *NS(=O)(=O)c1ccc(Nc2ncc(C(F)(F)F)c(N3CCCC(C)(O)C3)n2)c(C)c1]

LBM is selected from any of those in Table A below, and L is selected from any of those in Table B below.

In some embodiments, a provided compound or pharmaceutically acceptable salt thereof, is selected from those wherein CBM is

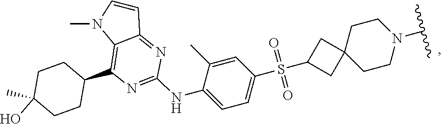 [SMILES: *N1CCC2(CC1)CC(S(=O)(=O)c1ccc(Nc3nc4ccn(C)c4c([C@H]4CC[C@@](C)(O)CC4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

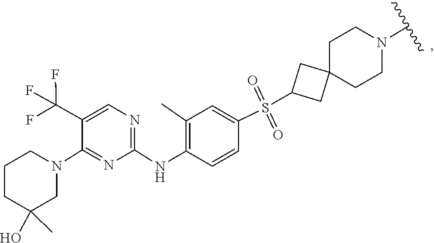 [SMILES: *N1CCC2(CC1)CC(S(=O)(=O)c1ccc(Nc3ncc(C(F)(F)F)c(N4CCCC(C)(O)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

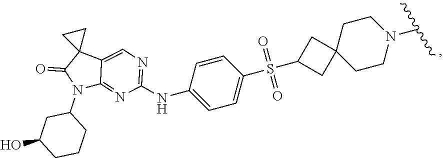 [SMILES: *N1CCC2(CC1)CC(S(=O)(=O)c1ccc(Nc3ncc4c(n3)N(C3CCC[C@@H](O)C3)C(=O)C43CC3)cc1)C2]

LBM is selected from any of those in Table A below, and L is selected from any of those in Table B below.

In some embodiments, a provided compound or pharmaceutically acceptable salt thereof, is selected from those wherein CBM is

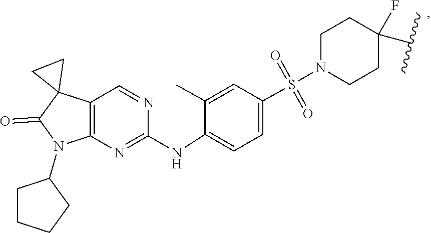 [SMILES: *C1(F)CCN(S(=O)(=O)c2ccc(Nc3ncc4c(n3)N(C3CCCC3)C(=O)C43CC3)c(C)c2)CC1]

LBM is selected from any of those in Table A below, and L is selected from any of those in Table B below.

In some embodiments, a provided compound or pharmaceutically acceptable salt thereof, is selected from those wherein CBM is

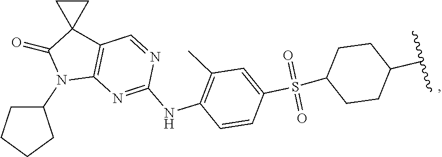 [SMILES: *C1CCC(S(=O)(=O)c2ccc(Nc3ncc4c(n3)N(C3CCCC3)C(=O)C43CC3)c(C)c2)CC1]

LBM is selected from any of those in Table A below, and L is selected from any of those in Table B below.

In some embodiments, a provided compound or pharmaceutically acceptable salt thereof, is selected from those wherein CBM is

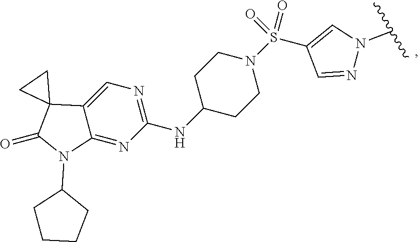 [SMILES: *n1cc(S(=O)(=O)N2CCC(Nc3ncc4c(n3)N(C3CCCC3)C(=O)C43CC3)CC2)cn1]

LBM is selected from any of those in Table A below, and L is selected from any of those in Table B below.

In some embodiments, a provided compound or pharmaceutically acceptable salt thereof, is selected from those wherein CBM is

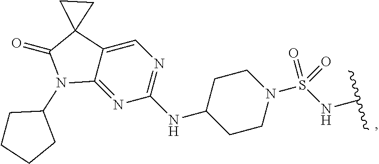 [SMILES: *NS(=O)(=O)N1CCC(Nc2ncc3c(n2)N(C2CCCC2)C(=O)C32CC2)CC1]

LBM is selected from any of those in Table A below, and L is selected from any of those in Table B below.

In some embodiments, a provided compound or pharmaceutically acceptable salt thereof, is selected from those wherein CBM is

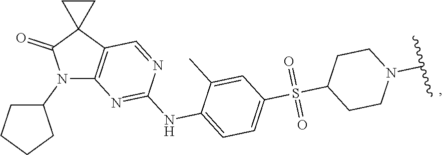 [SMILES: *N1CCC(S(=O)(=O)c2ccc(Nc3ncc4c(n3)N(C3CCCC3)C(=O)C43CC3)c(C)c2)CC1]

LBM is selected from any of those in Table A below, and L is selected from any of those in Table B below.

In some embodiments, a provided compound or pharmaceutically acceptable salt thereof, is selected from those wherein CBM is

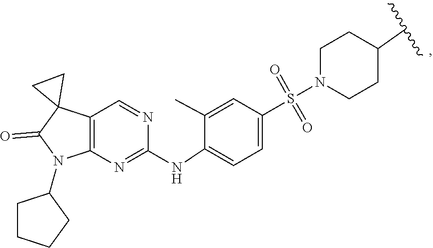 [SMILES: *C1CCN(S(=O)(=O)c2ccc(Nc3ncc4c(n3)N(C3CCCC3)C(=O)C43CC3)c(C)c2)CC1]

LBM is selected from any of those in Table A below, and L is selected from any of those in Table B below.

In some embodiments, a provided compound or pharmaceutically acceptable salt thereof, is selected from those wherein CBM is

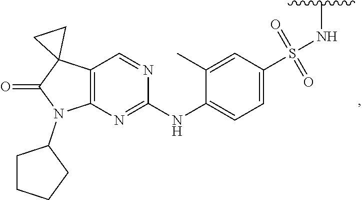 [SMILES: *NS(=O)(=O)c1ccc(Nc2ncc3c(n2)N(C2CCCC2)C(=O)C32CC2)c(C)c1]

LBM is selected from any of those in Table A below, and L is selected from any of those in Table B below.

In some embodiments, a provided compound or pharmaceutically acceptable salt thereof, is selected from those wherein CBM is

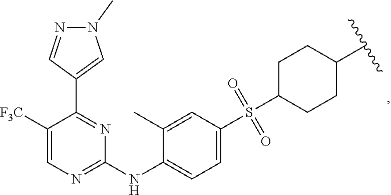 [SMILES: *C1CCC(S(=O)(=O)c2ccc(Nc3ncc(C(F)(F)F)c(-c4cnn(C)c4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

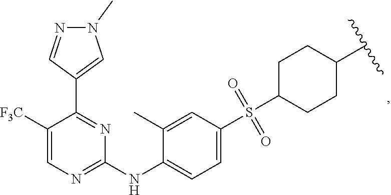 [SMILES: *C1CCC(S(=O)(=O)c2ccc(Nc3ncc(C(F)(F)F)c(-c4cnn(C)c4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

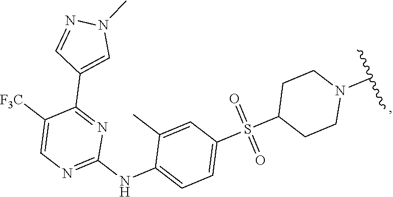 [SMILES: *N1CCC(S(=O)(=O)c2ccc(Nc3ncc(C(F)(F)F)c(-c4cnn(C)c4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

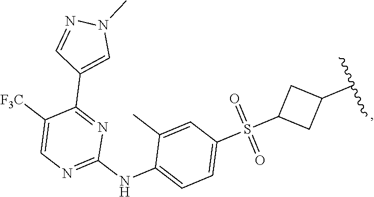 [SMILES: *C1CC(S(=O)(=O)c2ccc(Nc3ncc(C(F)(F)F)c(-c4cnn(C)c4)n3)c(C)c2)C1]

LBM is selected from any of those in Table A below, and L is selected from any of those in Table B below.

In some embodiments, a provided compound or pharmaceutically acceptable salt thereof, is selected from those wherein CBM is

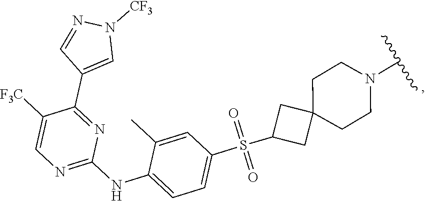 [SMILES: *N1CCC2(CC1)CC(S(=O)(=O)c1ccc(Nc3ncc(C(F)(F)F)c(-c4cnn(C(F)(F)F)c4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

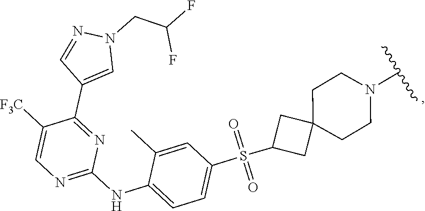 [SMILES: *N1CCC2(CC1)CC(S(=O)(=O)c1ccc(Nc3ncc(C(F)(F)F)c(-c4cnn(CC(F)F)c4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

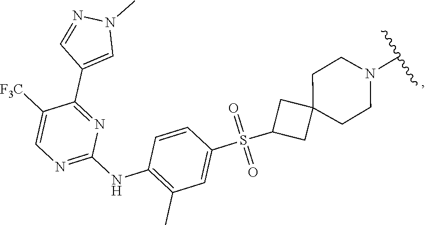 [SMILES: *N1CCC2(CC1)CC(S(=O)(=O)c1ccc(Nc3ncc(C(F)(F)F)c(-c4cnn(C)c4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

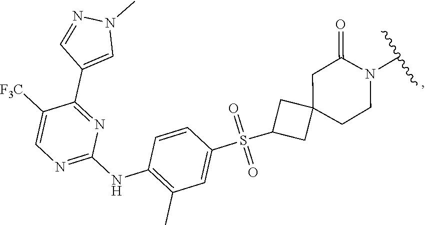 [SMILES: *N1CCC2(CC1=O)CC(S(=O)(=O)c1ccc(Nc3ncc(C(F)(F)F)c(-c4cnn(C)c4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

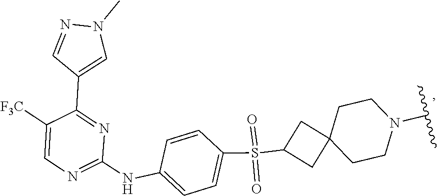 [SMILES: *N1CCC2(CC1)CC(S(=O)(=O)c1ccc(Nc3ncc(C(F)(F)F)c(-c4cnn(C)c4)n3)cc1)C2]

LBM is selected from any of those in Table A below, and L is selected from any of those in Table B below.

In some embodiments, a provided compound or pharmaceutically acceptable salt thereof, is selected from those wherein CBM is

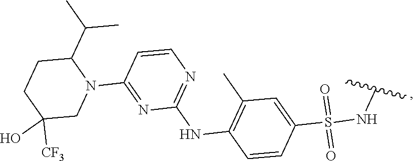 [SMILES: *NS(=O)(=O)c1ccc(Nc2nccc(N3CC(O)(C(F)(F)F)CCC3C(C)C)n2)c(C)c1]

LBM is selected from any of those in Table A below, and L is selected from any of those in Table B below.

In some embodiments, a provided compound or pharmaceutically acceptable salt thereof, is selected from those wherein CBM is

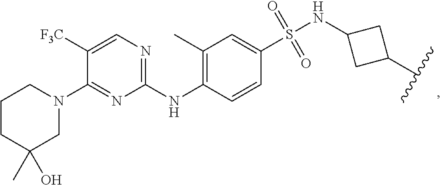 [SMILES: *C1CC(NS(=O)(=O)c2ccc(Nc3ncc(C(F)(F)F)c(N4CCCC(C)(O)C4)n3)c(C)c2)C1]

LBM is selected from any of those in Table A below, and L is selected from any of those in Table B below.

In some embodiments, a provided compound or pharmaceutically acceptable salt thereof, is selected from those wherein CBM is

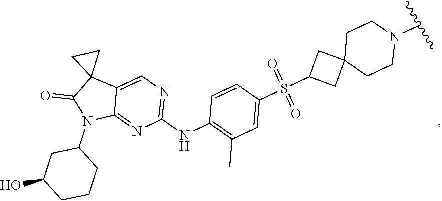 [SMILES: *N1CCC2(CC1)CC(S(=O)(=O)c1ccc(Nc3ncc4c(n3)N(C3CCC[C@@H](O)C3)C(=O)C43CC3)c(C)c1)C2]

LBM is selected from any of those in Table A below, and L is selected from any of those in Table B below.

In some embodiments, a provided compound or pharmaceutically acceptable salt thereof, is selected from those wherein CBM is

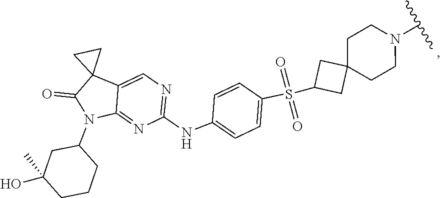 [SMILES: *N1CCC2(CC1)CC(S(=O)(=O)c1ccc(Nc3ncc4c(n3)N(C3CCC[C@@](C)(O)C3)C(=O)C43CC3)cc1)C2]

LBM is selected from any of those in Table A below, and L is selected from any of those in Table B below.

In some embodiments, a provided compound or pharmaceutically acceptable salt thereof, is selected from those wherein CBM is

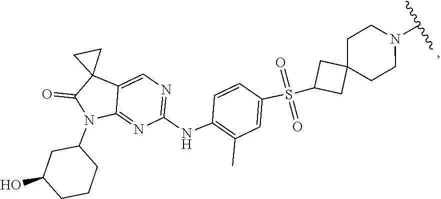 [SMILES: *N1CCC2(CC1)CC(S(=O)(=O)c1ccc(Nc3ncc4c(n3)N(C3CCC[C@@H](O)C3)C(=O)C43CC3)c(C)c1)C2]

LBM is selected from any of those in Table A below, and L is selected from any of those in Table B below.

In some embodiments, a provided compound or pharmaceutically acceptable salt thereof, is selected from those wherein CBM is

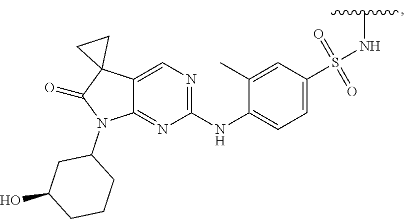 [SMILES: *NS(=O)(=O)c1ccc(Nc2ncc3c(n2)N(C2CCC[C@@H](O)C2)C(=O)C32CC2)c(C)c1]

LBM is selected from any of those in Table A below, and L is selected from any of those in Table B below.

In some embodiments, a provided compound or pharmaceutically acceptable salt thereof, is selected from those wherein CBM is

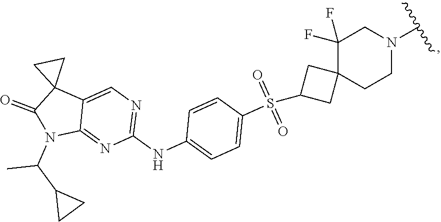 [SMILES: *N1CCC2(CC(S(=O)(=O)c3ccc(Nc4ncc5c(n4)N(C(C)C4CC4)C(=O)C54CC4)cc3)C2)C(F)(F)C1]

LBM is selected from any of those in Table A below, and L is selected from any of those in Table B below.

In some embodiments, a provided compound or pharmaceutically acceptable salt thereof, is selected from those wherein CBM is

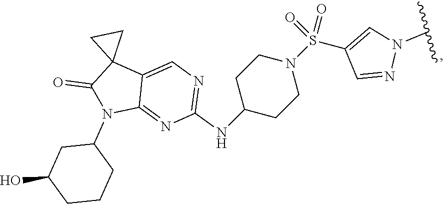 [SMILES: *n1cc(S(=O)(=O)N2CCC(Nc3ncc4c(n3)N(C3CCC[C@@H](O)C3)C(=O)C43CC3)CC2)cn1]

LBM is selected from any of those in Table A below, and L is selected from any of those in Table B below.

In some embodiments, a provided compound or pharmaceutically acceptable salt thereof, is selected from those wherein CBM is

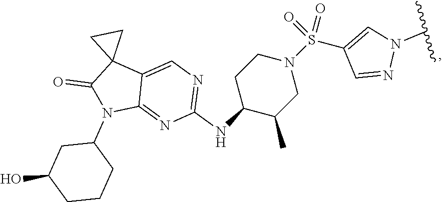 [SMILES: *n1cc(S(=O)(=O)N2CC[C@H](Nc3ncc4c(n3)N(C3CCC[C@@H](O)C3)C(=O)C43CC3)[C@H](C)C2)cn1]

LBM is selected from any of those in Table A below, and L is selected from any of those in Table B below.

In some embodiments, a provided compound or pharmaceutically acceptable salt thereof, is selected from those wherein CBM is

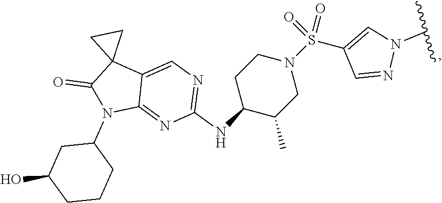 [SMILES: *n1cc(S(=O)(=O)N2CC[C@H](Nc3ncc4c(n3)N(C3CCC[C@@H](O)C3)C(=O)C43CC3)[C@@H](C)C2)cn1]

LBM is selected from any of those in Table A below, and L is selected from any of those in Table B below.

In some embodiments, a provided compound or pharmaceutically acceptable salt thereof, is selected from those wherein CBM is

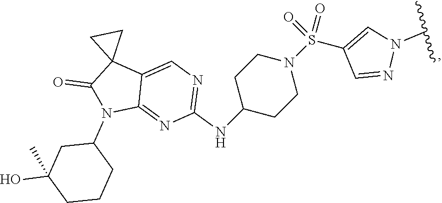 [SMILES: *n1cc(S(=O)(=O)N2CCC(Nc3ncc4c(n3)N(C3CCC[C@@](C)(O)C3)C(=O)C43CC3)CC2)cn1]

LBM is selected from any of those in Table A below, and L is selected from any of those in Table B below.

In some embodiments, a provided compound or pharmaceutically acceptable salt thereof, is selected from those wherein CBM is

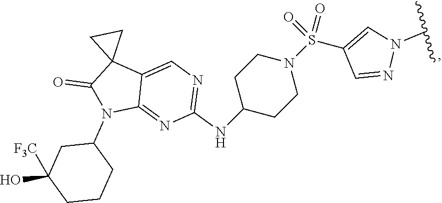 [SMILES: *n1cc(S(=O)(=O)N2CCC(Nc3ncc4c(n3)N(C3CCC[C@](O)(C(F)(F)F)C3)C(=O)C43CC3)CC2)cn1]

LBM is selected from any of those in Table A below, and L is selected from any of those in Table B below.

In some embodiments, a provided compound or pharmaceutically acceptable salt thereof, is selected from those wherein CBM is

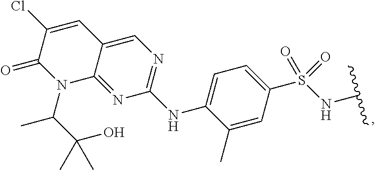 [SMILES: *NS(=O)(=O)c1ccc(Nc2ncc3cc(Cl)c(=O)n(C(C)C(C)(C)O)c3n2)c(C)c1]

LBM is selected from any of those in Table A below, and L is selected from any of those in Table B below.

In some embodiments, a provided compound or pharmaceutically acceptable salt thereof, is selected from those wherein CBM is

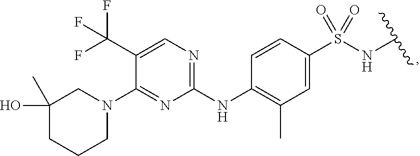 [SMILES: *NS(=O)(=O)c1ccc(Nc2ncc(C(F)(F)F)c(N3CCCC(C)(O)C3)n2)c(C)c1]

LBM is selected from any of those in Table A below, and L is selected from any of those in Table B below.

In some embodiments, a provided compound or pharmaceutically acceptable salt thereof, is selected from those wherein CBM is

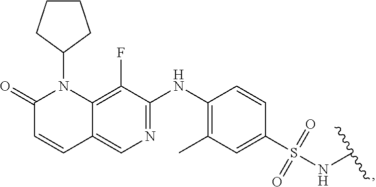 [SMILES: *NS(=O)(=O)c1ccc(Nc2ncc3ccc(=O)n(C4CCCC4)c3c2F)c(C)c1]

LBM is selected from any of those in Table A below, and L is selected from any of those in Table B below.

In some embodiments, a provided compound or pharmaceutically acceptable salt thereof, is selected from those wherein CBM is

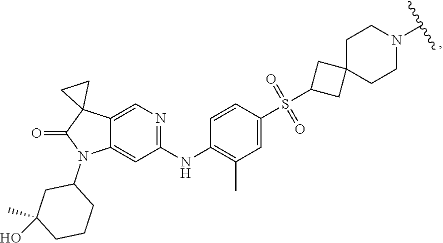 [SMILES: *N1CCC2(CC1)CC(S(=O)(=O)c1ccc(Nc3cc4c(cn3)C3(CC3)C(=O)N4C3CCC[C@@](C)(O)C3)c(C)c1)C2]

LBM is selected from any of those in Table A below, and L is selected from any of those in Table B below.

In some embodiments, a provided compound or pharmaceutically acceptable salt thereof, is selected from those wherein CBM is

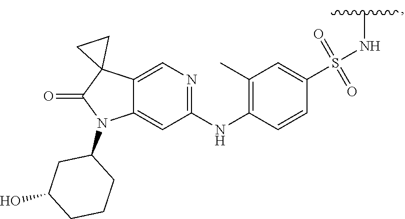 [SMILES: *NS(=O)(=O)c1ccc(Nc2cc3c(cn2)C2(CC2)C(=O)N3[C@H]2CCC[C@H](O)C2)c(C)c1]

LBM is selected from any of those in Table A below, and L is selected from any of those in Table B below.

In some embodiments, a provided compound or pharmaceutically acceptable salt thereof, is selected from those wherein CBM is

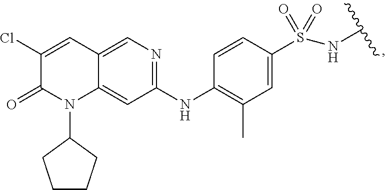 [SMILES: *NS(=O)(=O)c1ccc(Nc2cc3c(cn2)cc(Cl)c(=O)n3C2CCCC2)c(C)c1]

LBM is selected from any of those in Table A below, and L is selected from any of those in Table B below.

In some embodiments, a provided compound or pharmaceutically acceptable salt thereof, is selected from those wherein CBM is

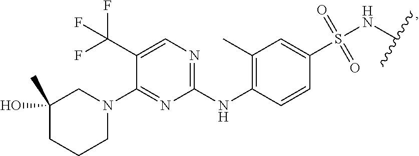 [SMILES: *NS(=O)(=O)c1ccc(Nc2ncc(C(F)(F)F)c(N3CCC[C@](C)(O)C3)n2)c(C)c1]

LBM is selected from any of those in Table A below, and L is selected from any of those in Table B below.

In some embodiments, a provided compound or pharmaceutically acceptable salt thereof, is selected from those wherein CBM is

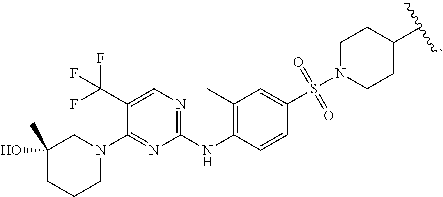 [SMILES: *C1CCN(S(=O)(=O)c2ccc(Nc3ncc(C(F)(F)F)c(N4CCC[C@](C)(O)C4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

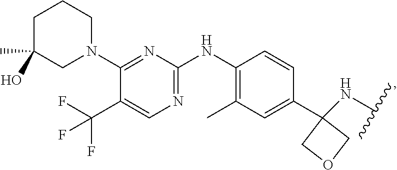 [SMILES: *NC1(c2ccc(Nc3ncc(C(F)(F)F)c(N4CCC[C@](C)(O)C4)n3)c(C)c2)COC1]

LBM is selected from any of those in Table A below, and L is selected from any of those in Table B below.

In some embodiments, a provided compound or pharmaceutically acceptable salt thereof, is selected from those wherein CBM is

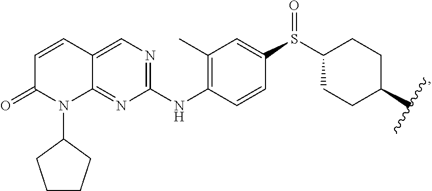 [SMILES: *[C@H]1CC[C@H]([S@](=O)c2ccc(Nc3ncc4ccc(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

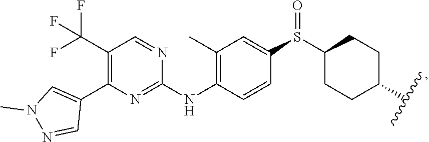 [SMILES: *[C@H]1CC[C@H]([S@](=O)c2ccc(Nc3ncc(C(F)(F)F)c(-c4cnn(C)c4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

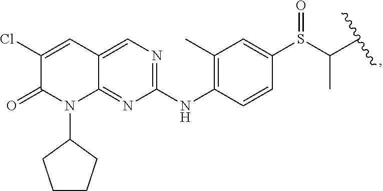 [SMILES: *C(C)S(=O)c1ccc(Nc2ncc3cc(Cl)c(=O)n(C4CCCC4)c3n2)c(C)c1]

LBM is selected from any of those in Table A below, and L is selected from any of those in Table B below.

In some embodiments, a provided compound or pharmaceutically acceptable salt thereof, is selected from those wherein CBM is

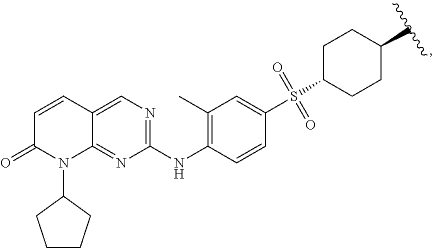 [SMILES: *[C@H]1CC[C@H](S(=O)(=O)c2ccc(Nc3ncc4ccc(=O)n(C5CCCC5)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

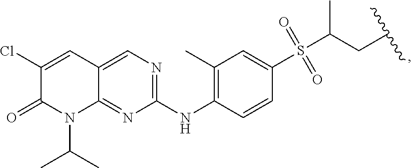 [SMILES: *CC(C)S(=O)(=O)c1ccc(Nc2ncc3cc(Cl)c(=O)n(C(C)C)c3n2)c(C)c1]

LBM is selected from any of those in Table A below, and L is selected from any of those in Table B below.

In some embodiments, a provided compound or pharmaceutically acceptable salt thereof, is selected from those wherein CBM is

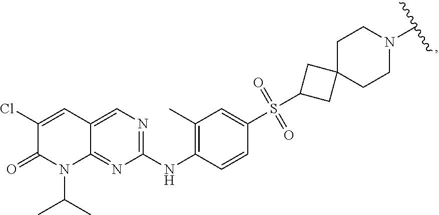 [SMILES: *N1CCC2(CC1)CC(S(=O)(=O)c1ccc(Nc3ncc4cc(Cl)c(=O)n(C(C)C)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

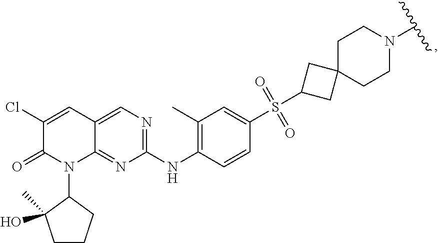 [SMILES: *N1CCC2(CC1)CC(S(=O)(=O)c1ccc(Nc3ncc4cc(Cl)c(=O)n(C5CCC[C@@]5(C)O)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

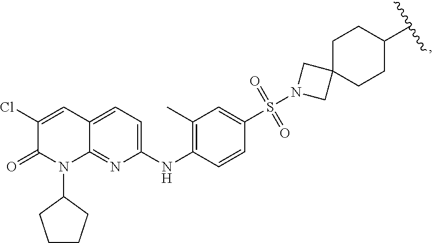 [SMILES: *C1CCC2(CC1)CN(S(=O)(=O)c1ccc(Nc3ccc4cc(Cl)c(=O)n(C5CCCC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

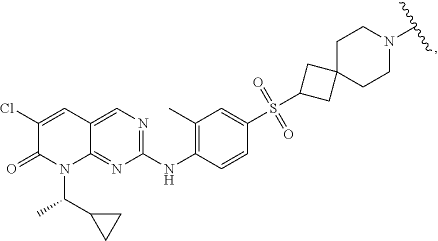 [SMILES: *N1CCC2(CC1)CC(S(=O)(=O)c1ccc(Nc3ncc4cc(Cl)c(=O)n([C@@H](C)C5CC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

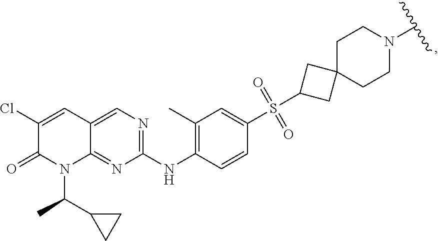 [SMILES: *N1CCC2(CC1)CC(S(=O)(=O)c1ccc(Nc3ncc4cc(Cl)c(=O)n([C@H](C)C5CC5)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

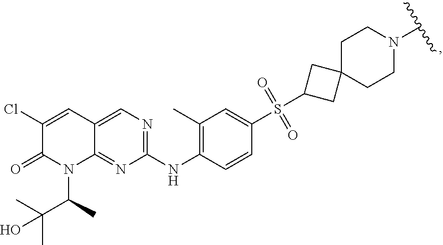 [SMILES: *N1CCC2(CC1)CC(S(=O)(=O)c1ccc(Nc3ncc4cc(Cl)c(=O)n([C@@H](C)C(C)(C)O)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

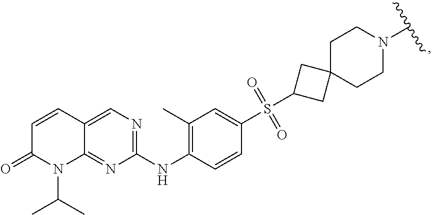 [SMILES: *N1CCC2(CC1)CC(S(=O)(=O)c1ccc(Nc3ncc4ccc(=O)n(C(C)C)c4n3)c(C)c1)C2]

LBM is selected from any of those in Table A below, and L is selected from any of those in Table B below.

In some embodiments, a provided compound or pharmaceutically acceptable salt thereof, is selected from those wherein CBM is

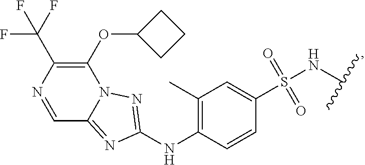 [SMILES: *NS(=O)(=O)c1ccc(Nc2nc3cnc(C(F)(F)F)c(OC4CCC4)n3n2)c(C)c1]

LBM is selected from any of those in Table A below, and L is selected from any of those in Table B below.

In some embodiments, a provided compound or pharmaceutically acceptable salt thereof, is selected from those wherein CBM is

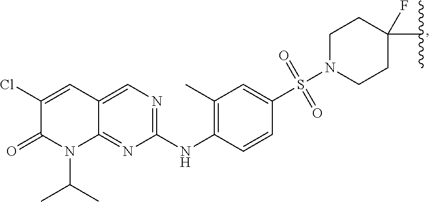 [SMILES: *C1(F)CCN(S(=O)(=O)c2ccc(Nc3ncc4cc(Cl)c(=O)n(C(C)C)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

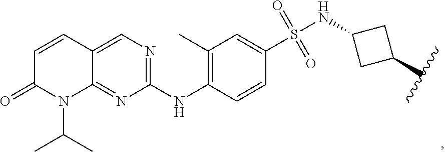 [SMILES: *[C@H]1C[C@H](NS(=O)(=O)c2ccc(Nc3ncc4ccc(=O)n(C(C)C)c4n3)c(C)c2)C1]

LBM is selected from any of those in Table A below, and L is selected from any of those in Table B below.

In some embodiments, a provided compound or pharmaceutically acceptable salt thereof, is selected from those wherein CBM is

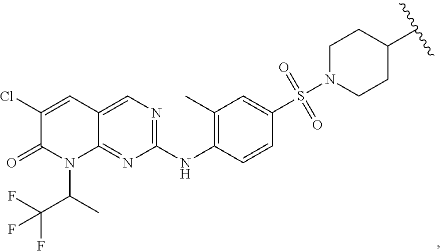 [SMILES: *C1CCN(S(=O)(=O)c2ccc(Nc3ncc4cc(Cl)c(=O)n(C(C)C(F)(F)F)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

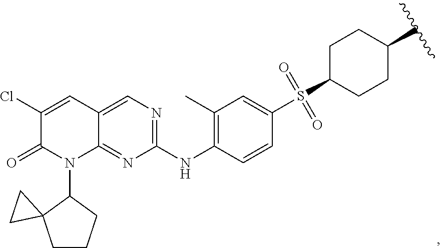 [SMILES: *[C@H]1CC[C@@H](S(=O)(=O)c2ccc(Nc3ncc4cc(Cl)c(=O)n(C5CCCC56CC6)c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

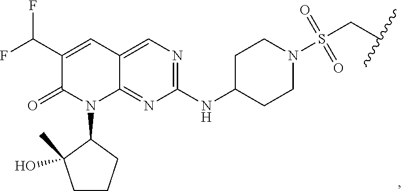 [SMILES: *CS(=O)(=O)N1CCC(Nc2ncc3cc(C(F)F)c(=O)n([C@H]4CCC[C@]4(C)O)c3n2)CC1]

LBM is selected from any of those in Table A below, and L is selected from any of those in Table B below.

In some embodiments, a provided compound or pharmaceutically acceptable salt thereof, is selected from those wherein CBM is

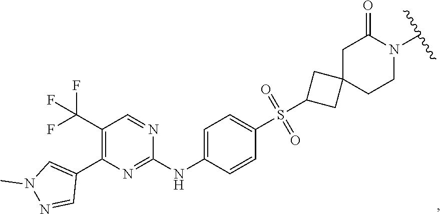 [SMILES: *N1CCC2(CC1=O)CC(S(=O)(=O)c1ccc(Nc3ncc(C(F)(F)F)c(-c4cnn(C)c4)n3)cc1)C2]

LBM is selected from any of those in Table A below, and L is selected from any of those in Table B below.

In some embodiments, a provided compound or pharmaceutically acceptable salt thereof, is selected from those wherein CBM is

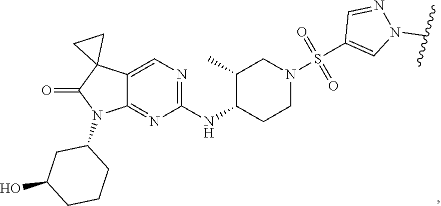 [SMILES: *n1cc(S(=O)(=O)N2CC[C@H](Nc3ncc4c(n3)N([C@@H]3CCC[C@@H](O)C3)C(=O)C43CC3)[C@H](C)C2)cn1]

LBM is selected from any of those in Table A below, and L is selected from any of those in Table B below.

In some embodiments, a provided compound or pharmaceutically acceptable salt thereof, is selected from those wherein CBM is

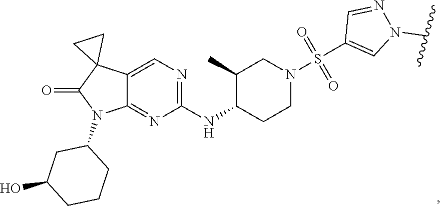 [SMILES: *n1cc(S(=O)(=O)N2CC[C@H](Nc3ncc4c(n3)N([C@@H]3CCC[C@@H](O)C3)C(=O)C43CC3)[C@@H](C)C2)cn1]

LBM is selected from any of those in Table A below, and L is selected from any of those in Table B below.

In some embodiments, a provided compound or pharmaceutically acceptable salt thereof, is selected from those wherein CBM is

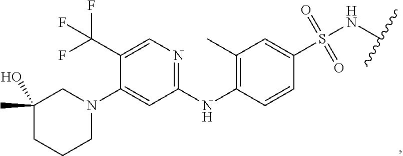 [SMILES: *NS(=O)(=O)c1ccc(Nc2cc(N3CCC[C@](C)(O)C3)c(C(F)(F)F)cn2)c(C)c1]

LBM is selected from any of those in Table A below, and L is selected from any of those in Table B below.

In some embodiments, a provided compound or pharmaceutically acceptable salt thereof, is selected from those wherein CBM is

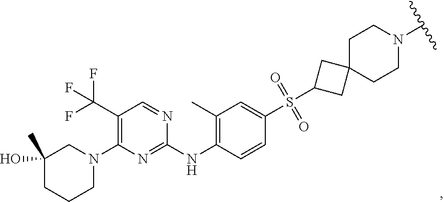 [SMILES: *N1CCC2(CC1)CC(S(=O)(=O)c1ccc(Nc3ncc(C(F)(F)F)c(N4CCC[C@](C)(O)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

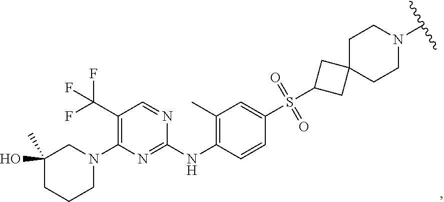 [SMILES: *N1CCC2(CC1)CC(S(=O)(=O)c1ccc(Nc3ncc(C(F)(F)F)c(N4CCC[C@@](C)(O)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

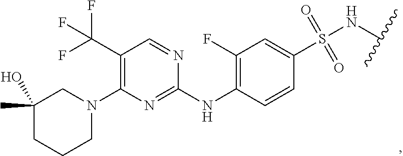 [SMILES: *NS(=O)(=O)c1ccc(Nc2ncc(C(F)(F)F)c(N3CCC[C@](C)(O)C3)n2)c(F)c1]

LBM is selected from any of those in Table A below, and L is selected from any of those in Table B below.

In some embodiments, a provided compound or pharmaceutically acceptable salt thereof, is selected from those wherein CBM is

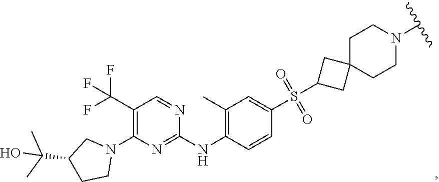 [SMILES: *N1CCC2(CC1)CC(S(=O)(=O)c1ccc(Nc3ncc(C(F)(F)F)c(N4CC[C@H](C(C)(C)O)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

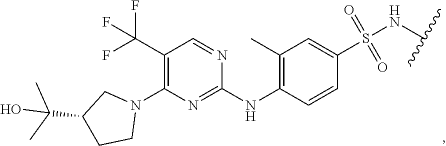 [SMILES: *NS(=O)(=O)c1ccc(Nc2ncc(C(F)(F)F)c(N3CC[C@H](C(C)(C)O)C3)n2)c(C)c1]

LBM is selected from any of those in Table A below, and L is selected from any of those in Table B below.

In some embodiments, a provided compound or pharmaceutically acceptable salt thereof, is selected from those wherein CBM is

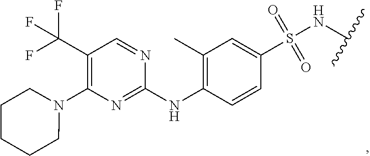 [SMILES: *NS(=O)(=O)c1ccc(Nc2ncc(C(F)(F)F)c(N3CCCCC3)n2)c(C)c1]

LBM is selected from any of those in Table A below, and L is selected from any of those in Table B below.

In some embodiments, a provided compound or pharmaceutically acceptable salt thereof, is selected from those wherein CBM is

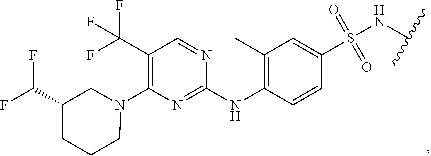 [SMILES: *NS(=O)(=O)c1ccc(Nc2ncc(C(F)(F)F)c(N3CCC[C@H](C(F)F)C3)n2)c(C)c1]

LBM is selected from any of those in Table A below, and L is selected from any of those in Table B below.

In some embodiments, a provided compound or pharmaceutically acceptable salt thereof, is selected from those wherein CBM is

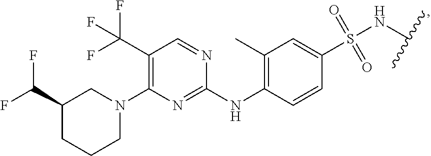 [SMILES: *NS(=O)(=O)c1ccc(Nc2ncc(C(F)(F)F)c(N3CCC[C@@H](C(F)F)C3)n2)c(C)c1]

LBM is selected from any of those in Table A below, and L is selected from any of those in Table B below.

In some embodiments, a provided compound or pharmaceutically acceptable salt thereof, is selected from those wherein CBM is

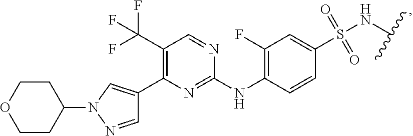 [SMILES: *NS(=O)(=O)c1ccc(Nc2ncc(C(F)(F)F)c(-c3cnn(C4CCOCC4)c3)n2)c(F)c1]

LBM is selected from any of those in Table A below, and L is selected from any of those in Table B below.

In some embodiments, a provided compound or pharmaceutically acceptable salt thereof, is selected from those wherein CBM is

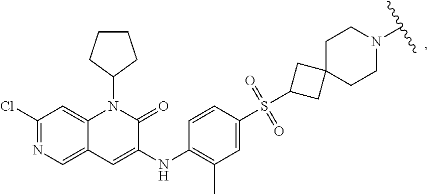 [SMILES: *N1CCC2(CC1)CC(S(=O)(=O)c1ccc(Nc3cc4cnc(Cl)cc4n(C4CCCC4)c3=O)c(C)c1)C2]

LBM is selected from any of those in Table A below, and L is selected from any of those in Table B below.

In some embodiments, a provided compound or pharmaceutically acceptable salt thereof, is selected from those wherein CBM is

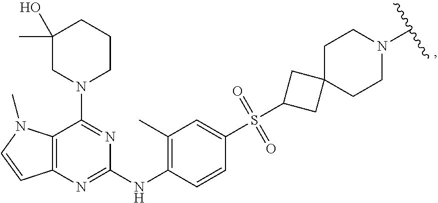 [SMILES: *N1CCC2(CC1)CC(S(=O)(=O)c1ccc(Nc3nc(N4CCCC(C)(O)C4)c4c(ccn4C)n3)c(C)c1)C2]

LBM is selected from any any of those in Table A below, and L is selected from any of those in Table B below.

In some embodiments, a provided compound or pharmaceutically acceptable salt thereof, is selected from those wherein CBM is

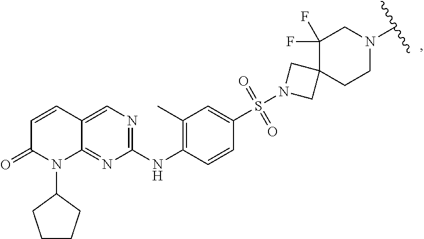 [SMILES: *N1CCC2(CN(S(=O)(=O)c3ccc(Nc4ncc5ccc(=O)n(C6CCCC6)c5n4)c(C)c3)C2)C(F)(F)C1]

LBM is selected from any of those in Table A below, and L is selected from any of those in Table B below.

In some embodiments, a provided compound or pharmaceutically acceptable salt thereof, is selected from those wherein CBM is

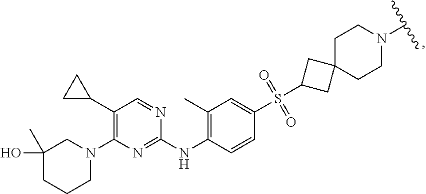 [SMILES: *N1CCC2(CC1)CC(S(=O)(=O)c1ccc(Nc3ncc(C4CC4)c(N4CCCC(C)(O)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

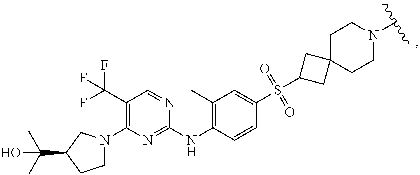 [SMILES: *N1CCC2(CC1)CC(S(=O)(=O)c1ccc(Nc3ncc(C(F)(F)F)c(N4CC[C@@H](C(C)(C)O)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

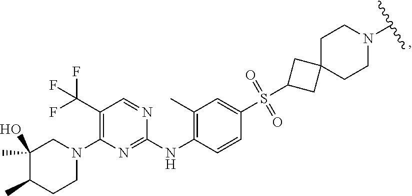 [SMILES: *N1CCC2(CC1)CC(S(=O)(=O)c1ccc(Nc3ncc(C(F)(F)F)c(N4CC[C@@H](C)[C@@](C)(O)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

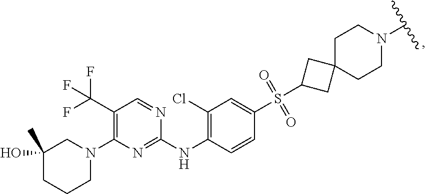 [SMILES: *N1CCC2(CC1)CC(S(=O)(=O)c1ccc(Nc3ncc(C(F)(F)F)c(N4CCC[C@](C)(O)C4)n3)c(Cl)c1)C2]

LBM is selected from any of those in Table A below, and L is selected from any of those in Table B below.

In some embodiments, a provided compound or pharmaceutically acceptable salt thereof, is selected from those wherein CBM is

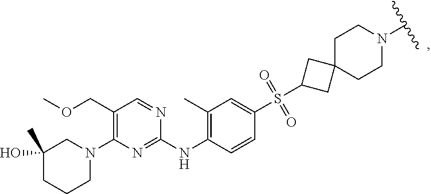 [SMILES: *N1CCC2(CC1)CC(S(=O)(=O)c1ccc(Nc3ncc(COC)c(N4CCC[C@](C)(O)C4)n3)c(C)c1)C2]

LBM is selected from any of those in Table A below, and L is selected from any of those in Table B below.

In some embodiments, a provided compound or pharmaceutically acceptable salt thereof, is selected from those wherein CBM is

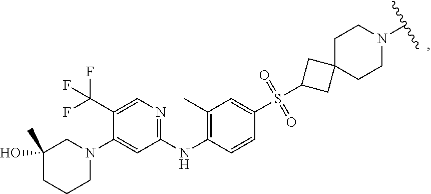 [SMILES: *N1CCC2(CC1)CC(S(=O)(=O)c1ccc(Nc3cc(N4CCC[C@](C)(O)C4)c(C(F)(F)F)cn3)c(C)c1)C2]

LBM is selected from any of those in Table A below, and L is selected from any of those in Table B below.

In some embodiments, a provided compound or pharmaceutically acceptable salt thereof, is selected from those wherein CBM is

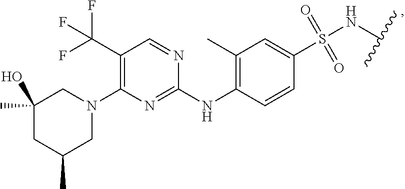 [SMILES: *NS(=O)(=O)c1ccc(Nc2ncc(C(F)(F)F)c(N3C[C@@H](C)C[C@@](C)(O)C3)n2)c(C)c1]

LBM is selected from any of those in Table A below, and L is selected from any of those in Table B below.

In some embodiments, a provided compound or pharmaceutically acceptable salt thereof, is selected from those wherein CBM is

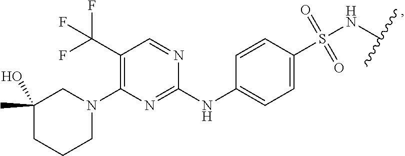 [SMILES: *NS(=O)(=O)c1ccc(Nc2ncc(C(F)(F)F)c(N3CCC[C@](C)(O)C3)n2)cc1]

LBM is selected from any of those in Table A below, and L is selected from any of those in Table B below.

In some embodiments, a provided compound or pharmaceutically acceptable salt thereof, is selected from those wherein CBM is

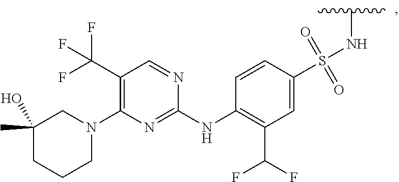 [SMILES: *NS(=O)(=O)c1ccc(Nc2ncc(C(F)(F)F)c(N3CCC[C@](C)(O)C3)n2)c(C(F)F)c1]

LBM is selected from any of those in Table A below, and L is selected from any of those in Table B below.

In some embodiments, a provided compound or pharmaceutically acceptable salt thereof, is selected from those wherein CBM is

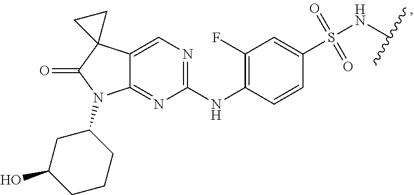 [SMILES: *NS(=O)(=O)c1ccc(Nc2ncc3c(n2)N([C@@H]2CCC[C@@H](O)C2)C(=O)C32CC2)c(F)c1]

LBM is selected from any of those in Table A below, and L is selected from any of those in Table B below.

In some embodiments, a provided compound or pharmaceutically acceptable salt thereof, is selected from those wherein CBM is

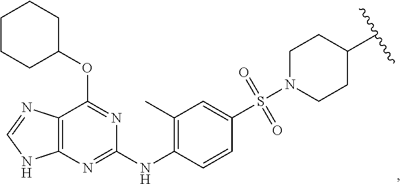 [SMILES: *C1CCN(S(=O)(=O)c2ccc(Nc3nc(OC4CCCCC4)c4nc[nH]c4n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

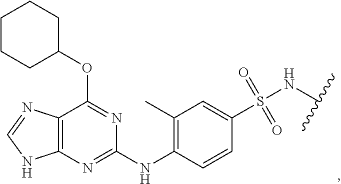 [SMILES: *NS(=O)(=O)c1ccc(Nc2nc(OC3CCCCC3)c3nc[nH]c3n2)c(C)c1]

LBM is selected from any of those in Table A below, and L is selected from any of those in Table B below.

In some embodiments, a provided compound or pharmaceutically acceptable salt thereof, is selected from those wherein CBM is

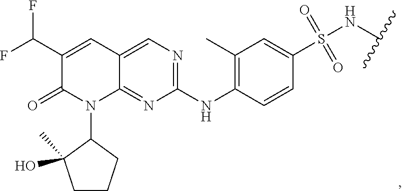 [SMILES: *NS(=O)(=O)c1ccc(Nc2ncc3cc(C(F)F)c(=O)n(C4CCC[C@@]4(C)O)c3n2)c(C)c1]

LBM is selected from any of those in Table A below, and L is selected from any of those in Table B below.

In some embodiments, a provided compound or pharmaceutically acceptable salt thereof, is selected from those wherein CBM is

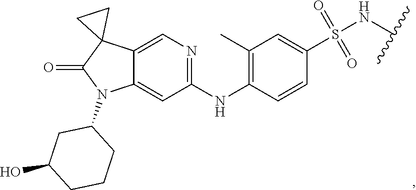 [SMILES: *NS(=O)(=O)c1ccc(Nc2cc3c(cn2)C2(CC2)C(=O)N3[C@@H]2CCC[C@@H](O)C2)c(C)c1]

LBM is selected from any of those in Table A below, and L is selected from any of those in Table B below.

In some embodiments, a provided compound or pharmaceutically acceptable salt thereof, is selected from those wherein CBM is

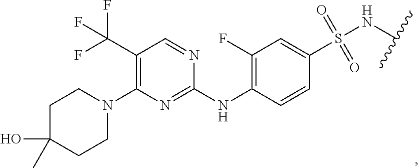 [SMILES: *NS(=O)(=O)c1ccc(Nc2ncc(C(F)(F)F)c(N3CCC(C)(O)CC3)n2)c(F)c1]

LBM is selected from any of those in Table A below, and L is selected from any of those in Table B below.

In some embodiments, a provided compound or pharmaceutically acceptable salt thereof, is selected from those wherein CBM is

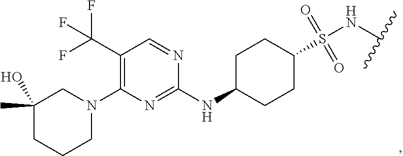 [SMILES: *NS(=O)(=O)[C@H]1CC[C@H](Nc2ncc(C(F)(F)F)c(N3CCC[C@](C)(O)C3)n2)CC1]

LBM is selected from any of those in Table A below, and L is selected from any of those in Table B below.

In some embodiments, a provided compound or pharmaceutically acceptable salt thereof, is selected from those wherein CBM is

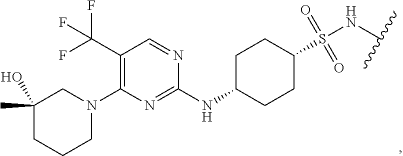 [SMILES: *NS(=O)(=O)[C@H]1CC[C@@H](Nc2ncc(C(F)(F)F)c(N3CCC[C@](C)(O)C3)n2)CC1]

LBM is selected from any of those in Table A below, and L is selected from any of those in Table B below.

In some embodiments, a provided compound or pharmaceutically acceptable salt thereof, is selected from those wherein CBM is

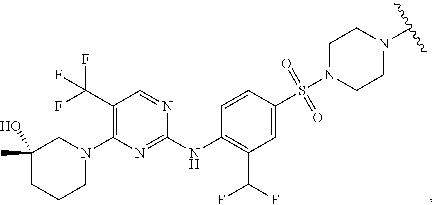 [SMILES: *N1CCN(S(=O)(=O)c2ccc(Nc3ncc(C(F)(F)F)c(N4CCC[C@](C)(O)C4)n3)c(C(F)F)c2)CC1]

LBM is selected from any of those in Table A below, and L is selected from any of those in Table B below.

In some embodiments, a provided compound or pharmaceutically acceptable salt thereof, is selected from those wherein CBM is

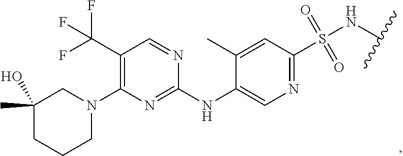 [SMILES: *NS(=O)(=O)c1cc(C)c(Nc2ncc(C(F)(F)F)c(N3CCC[C@](C)(O)C3)n2)cn1]

LBM is selected from any of those in Table A below, and L is selected from any of those in Table B below.

In some embodiments, a provided compound or pharmaceutically acceptable salt thereof, is selected from those wherein CBM is

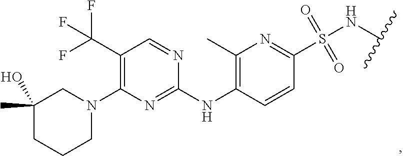 [SMILES: *NS(=O)(=O)c1ccc(Nc2ncc(C(F)(F)F)c(N3CCC[C@](C)(O)C3)n2)c(C)n1]

LBM is selected from any of those in Table A below, and L is selected from any of those in Table B below.

In some embodiments, a provided compound or pharmaceutically acceptable salt thereof, is selected from those wherein CBM is

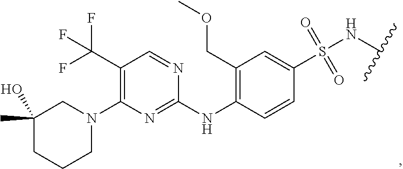 [SMILES: *NS(=O)(=O)c1ccc(Nc2ncc(C(F)(F)F)c(N3CCC[C@](C)(O)C3)n2)c(COC)c1]

LBM is selected from any of those in Table A below, and L is selected from any of those in Table B below.

In some embodiments, a provided compound or pharmaceutically acceptable salt thereof, is selected from those wherein CBM is

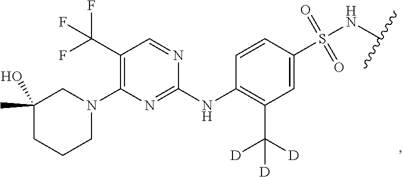 [SMILES: *NS(=O)(=O)c1ccc(Nc2ncc(C(F)(F)F)c(N3CCC[C@](C)(O)C3)n2)c(C([2H])([2H])[2H])c1]

LBM is selected from any of those in Table A below, and L is selected from any of those in Table B below.

In some embodiments, a provided compound or pharmaceutically acceptable salt thereof, is selected from those wherein CBM is

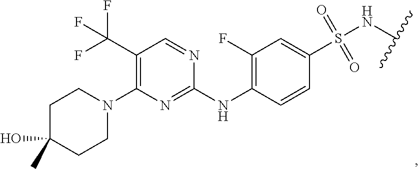

LBM is selected from any of those in Table A below, and L is selected from any of those in Table B below.

In some embodiments, a provided compound or pharmaceutically acceptable salt thereof, is selected from those wherein CBM is

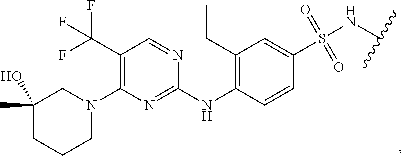 [SMILES: *NS(=O)(=O)c1ccc(Nc2ncc(C(F)(F)F)c(N3CCC[C@](C)(O)C3)n2)c(CC)c1]

LBM is selected from any of those in Table A below, and L is selected from any of those in Table B below.

In some embodiments, a provided compound or pharmaceutically acceptable salt thereof, is selected from those wherein CBM is

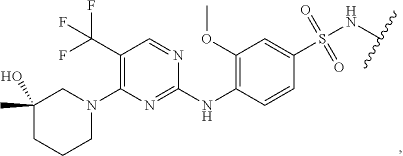 [SMILES: *NS(=O)(=O)c1ccc(Nc2ncc(C(F)(F)F)c(N3CCC[C@](C)(O)C3)n2)c(OC)c1]

LBM is selected from any of those in Table A below, and L is selected from any of those in Table B below.

In some embodiments, a provided compound or pharmaceutically acceptable salt thereof, is selected from those wherein CBM is

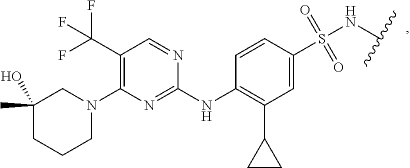 [SMILES: *NS(=O)(=O)c1ccc(Nc2ncc(C(F)(F)F)c(N3CCC[C@](C)(O)C3)n2)c(C2CC2)c1]

LBM is selected from any of those in Table A below, and L is selected from any of those in Table B below.

In some embodiments, a provided compound or pharmaceutically acceptable salt thereof, is selected from those wherein CBM is

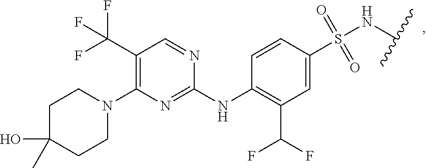 [SMILES: *NS(=O)(=O)c1ccc(Nc2ncc(C(F)(F)F)c(N3CCC(C)(O)CC3)n2)c(C(F)F)c1]

LBM is selected from any of those in Table A below, and L is selected from any of those in Table B below.

In some embodiments, a provided compound or pharmaceutically acceptable salt thereof, is selected from those wherein CBM is

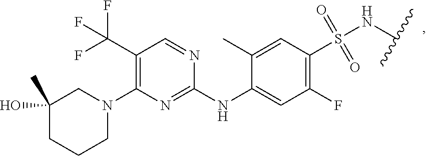 [SMILES: *NS(=O)(=O)c1cc(C)c(Nc2ncc(C(F)(F)F)c(N3CCC[C@](C)(O)C3)n2)cc1F]

LBM is selected from any of those in Table A below, and L is selected from any of those in Table B below.

In some embodiments, a provided compound or pharmaceutically acceptable salt thereof, is selected from those wherein CBM is

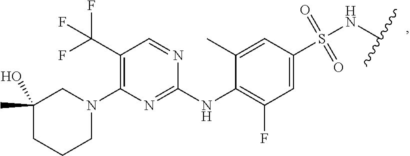 [SMILES: *NS(=O)(=O)c1cc(C)c(Nc2ncc(C(F)(F)F)c(N3CCC[C@](C)(O)C3)n2)c(F)c1]

LBM is selected from any of those in Table A below, and L is selected from any of those in Table B below.

In some embodiments, a provided compound or pharmaceutically acceptable salt thereof, is selected from those wherein CBM is

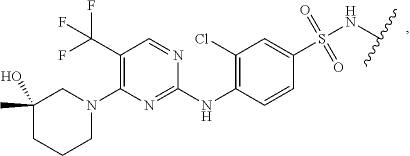 [SMILES: *NS(=O)(=O)c1ccc(Nc2ncc(C(F)(F)F)c(N3CCC[C@](C)(O)C3)n2)c(Cl)c1]

LBM is selected from any of those in Table A below, and L is selected from any of those in Table B below.

In some embodiments, a provided compound or pharmaceutically acceptable salt thereof, is selected from those wherein CBM is

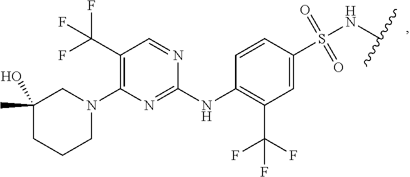 [SMILES: *NS(=O)(=O)c1ccc(Nc2ncc(C(F)(F)F)c(N3CCC[C@](C)(O)C3)n2)c(C(F)(F)F)c1]

LBM is selected from any of those in Table A below, and L is selected from any of those in Table B below.

In some embodiments, a provided compound or pharmaceutically acceptable salt thereof, is selected from those wherein CBM is

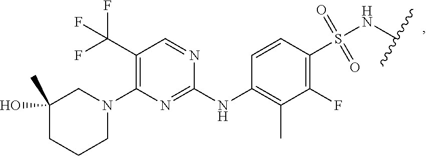 [SMILES: *NS(=O)(=O)c1ccc(Nc2ncc(C(F)(F)F)c(N3CCC[C@](C)(O)C3)n2)c(C)c1F]

LBM is selected from any of those in Table A below, and L is selected from any of those in Table B below.

In some embodiments, a provided compound or pharmaceutically acceptable salt thereof, is selected from those wherein CBM is

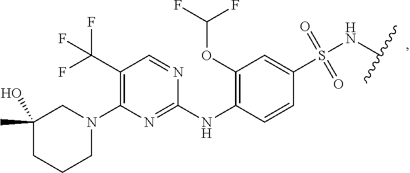 [SMILES: *NS(=O)(=O)c1ccc(Nc2ncc(C(F)(F)F)c(N3CCC[C@](C)(O)C3)n2)c(OC(F)F)c1]

LBM is selected from any of those in Table A below, and L is selected from any of those in Table B below.

In some embodiments, a provided compound or pharmaceutically acceptable salt thereof, is selected from those wherein CBM is

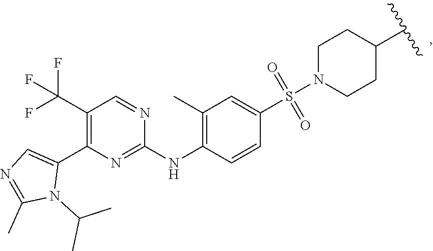 [SMILES: *C1CCN(S(=O)(=O)c2ccc(Nc3ncc(C(F)(F)F)c(-c4cnc(C)n4C(C)C)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

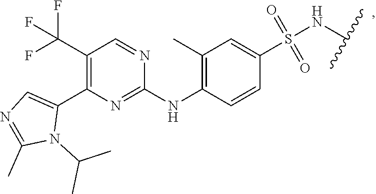 [SMILES: *NS(=O)(=O)c1ccc(Nc2ncc(C(F)(F)F)c(-c3cnc(C)n3C(C)C)n2)c(C)c1]

LBM is selected from any of those in Table A below, and L is selected from any of those in Table B below.

In some embodiments, a provided compound or pharmaceutically acceptable salt thereof, is selected from those wherein CBM is

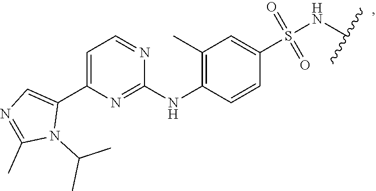 [SMILES: *NS(=O)(=O)c1ccc(Nc2nccc(-c3cnc(C)n3C(C)C)n2)c(C)c1]

LBM is selected from any of those in Table A below, and L is selected from any of those in Table B below.

In some embodiments, a provided compound or pharmaceutically acceptable salt thereof, is selected from those wherein CBM is

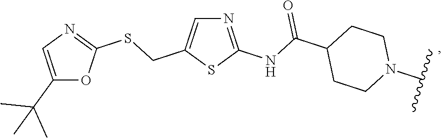 [SMILES: *N1CCC(C(=O)Nc2ncc(CSc3ncc(C(C)(C)C)o3)s2)CC1]

LBM is selected from any of those in Table A below, and L is selected from any of those in Table B below.

In some embodiments, a provided compound or pharmaceutically acceptable salt thereof, is selected from those wherein CBM is

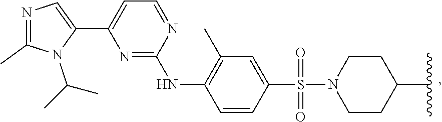 [SMILES: *C1CCN(S(=O)(=O)c2ccc(Nc3nccc(-c4cnc(C)n4C(C)C)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

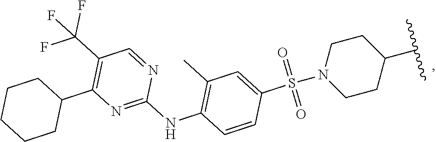 [SMILES: *C1CCN(S(=O)(=O)c2ccc(Nc3ncc(C(F)(F)F)c(C4CCCCC4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

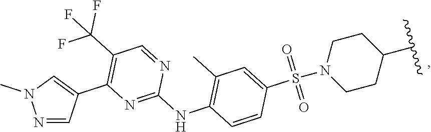 [SMILES: *C1CCN(S(=O)(=O)c2ccc(Nc3ncc(C(F)(F)F)c(-c4cnn(C)c4)n3)c(C)c2)CC1]

LBM is selected from any of those in Table A below, and L is selected from any of those in Table B below.

In some embodiments, a provided compound or pharmaceutically acceptable salt thereof, is selected from those wherein CBM is

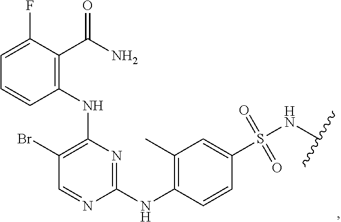 [SMILES: *NS(=O)(=O)c1ccc(Nc2ncc(Br)c(Nc3cccc(F)c3C(N)=O)n2)c(C)c1]

LBM is selected from any of those in Table A below, and L is selected from any of those in Table B below.

In some embodiments, a provided compound or pharmaceutically acceptable salt thereof, is selected from those wherein CBM is

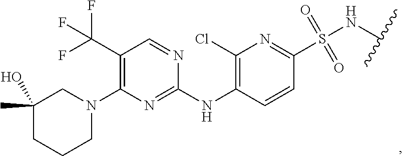 [SMILES: *NS(=O)(=O)c1ccc(Nc2ncc(C(F)(F)F)c(N3CCC[C@](C)(O)C3)n2)c(Cl)n1]

LBM is selected from any of those in Table A below, and L is selected from any of those in Table B below.

In some embodiments, a provided compound or pharmaceutically acceptable salt thereof, is selected from those wherein CBM is

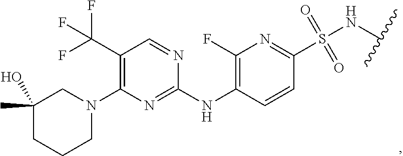 [SMILES: *NS(=O)(=O)c1ccc(Nc2ncc(C(F)(F)F)c(N3CCC[C@](C)(O)C3)n2)c(F)n1]

LBM is selected from any of those in Table A below, and L is selected from any of those in Table B below.

In some embodiments, a provided compound or pharmaceutically acceptable salt thereof, is selected from those wherein CBM is

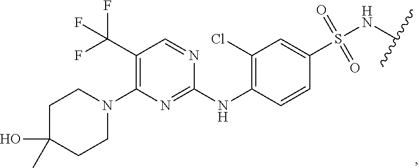 [SMILES: *NS(=O)(=O)c1ccc(Nc2ncc(C(F)(F)F)c(N3CCC(C)(O)CC3)n2)c(Cl)c1]

LBM is selected from any of those in Table A below, and L is selected from any of those in Table B below.

In some embodiments, a provided compound or pharmaceutically acceptable salt thereof, is selected from those wherein CBM is

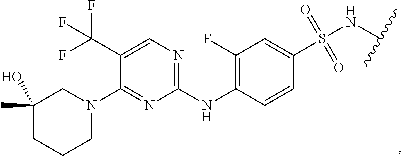 [SMILES: *NS(=O)(=O)c1ccc(Nc2ncc(C(F)(F)F)c(N3CCC[C@](C)(O)C3)n2)c(F)c1]

LBM is selected from any of those in Table A below, and L is selected from any of those in Table B below.

In some embodiments, a provided compound or pharmaceutically acceptable salt thereof, is selected from those wherein CBM is

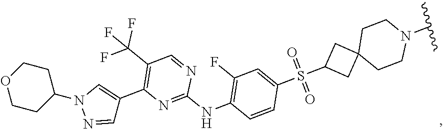 [SMILES: *N1CCC2(CC1)CC(S(=O)(=O)c1ccc(Nc3ncc(C(F)(F)F)c(-c4cnn(C5CCOCC5)c4)n3)c(F)c1)C2]

LBM is selected from any of those in Table A below, and L is selected from any of those in Table B below.

In some embodiments, a provided compound or pharmaceutically acceptable salt thereof, is selected from those wherein CBM is

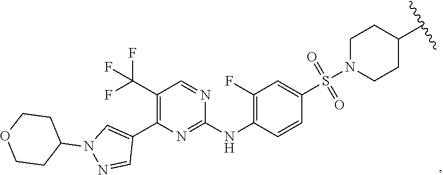 [SMILES: *C1CCN(S(=O)(=O)c2ccc(Nc3ncc(C(F)(F)F)c(-c4cnn(C5CCOCC5)c4)n3)c(F)c2)CC1]

LBM is selected from any of those in Table A below, and L is selected from any of those in Table B below.

In some embodiments, a provided compound or pharmaceutically acceptable salt thereof, is selected from those wherein CBM is

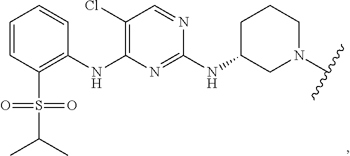 [SMILES: *N1CCC[C@@H](Nc2ncc(Cl)c(Nc3ccccc3S(=O)(=O)C(C)C)n2)C1]

LBM is selected from any of those in Table A below, and L is selected from any of those in Table B below.

In some embodiments, a provided compound or pharmaceutically acceptable salt thereof, is selected from those wherein CBM is

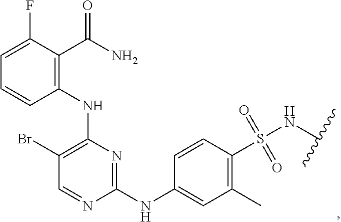 [SMILES: *NS(=O)(=O)c1ccc(Nc2ncc(Br)c(Nc3cccc(F)c3C(N)=O)n2)cc1C]

LBM is selected from any of those in Table A below, and L is selected from any of those in Table B below.

In some embodiments, a provided compound or pharmaceutically acceptable salt thereof, is selected from those wherein CBM is

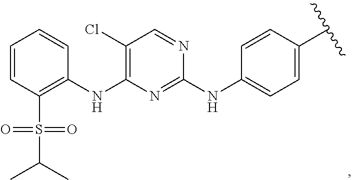 [SMILES: *c1ccc(Nc2ncc(Cl)c(Nc3ccccc3S(=O)(=O)C(C)C)n2)cc1]

LBM is selected from any of those in Table A below, and L is selected from any of those in Table B below.

In some embodiments, a provided compound or pharmaceutically acceptable salt thereof, is selected from those wherein CBM is

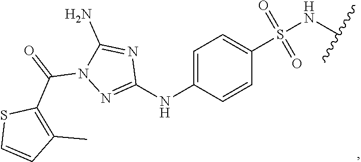 [SMILES: *NS(=O)(=O)c1ccc(Nc2nc(N)n(C(=O)c3sccc3C)n2)cc1]

LBM is selected from any of those in Table A below, and L is selected from any of those in Table B below.

In some embodiments, a provided compound or pharmaceutically acceptable salt thereof, is selected from those wherein CBM is

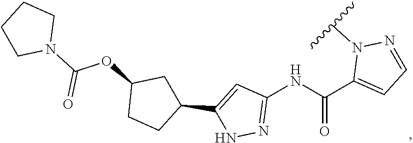 [SMILES: *n1nccc1C(=O)Nc1cc([C@H]2CC[C@@H](OC(=O)N3CCCC3)C2)[nH]n1]

LBM is selected from any of those in Table A below, and L is selected from any of those in Table B below.

In some embodiments, a provided compound or pharmaceutically acceptable salt thereof, is selected from those wherein CBM is

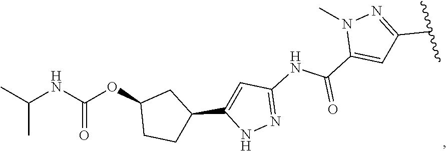 [SMILES: *c1cc(C(=O)Nc2cc([C@H]3CC[C@@H](OC(=O)NC(C)C)C3)[nH]n2)n(C)n1]

LBM is selected from any of those in Table A below, and L is selected from any of those in Table B below.

In some embodiments, a provided compound or pharmaceutically acceptable salt thereof, is selected from those wherein CBM is

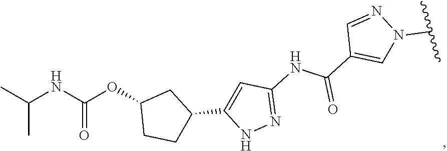 [SMILES: *n1cc(C(=O)Nc2cc([C@@H]3CC[C@H](OC(=O)NC(C)C)C3)[nH]n2)cn1]

LBM is selected from any of those in Table A below, and L is selected from any of those in Table B below.

In some embodiments, a provided compound or pharmaceutically acceptable salt thereof, is selected from those wherein CBM is

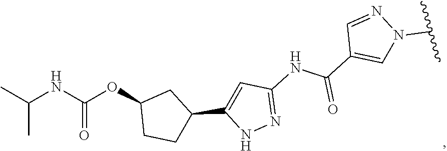 [SMILES: *n1cc(C(=O)Nc2cc([C@H]3CC[C@@H](OC(=O)NC(C)C)C3)[nH]n2)cn1]

LBM is selected from any of those in Table A below, and L is selected from any of those in Table B below.

In some embodiments, a provided compound or pharmaceutically acceptable salt thereof, is selected from those wherein CBM is

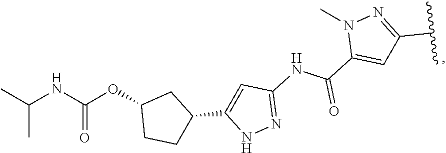 [SMILES: *c1cc(C(=O)Nc2cc([C@@H]3CC[C@H](OC(=O)NC(C)C)C3)[nH]n2)n(C)n1]

LBM is selected from any of those in Table A below, and L is selected from any of those in Table B below.

In some embodiments, a provided compound or pharmaceutically acceptable salt thereof, is selected from those wherein CBM is

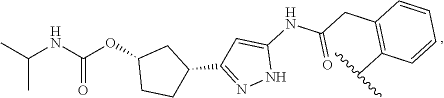 [SMILES: *c1ccccc1CC(=O)Nc1cc([C@@H]2CC[C@H](OC(=O)NC(C)C)C2)n[nH]1]

LBM is selected from any of those in Table A below, and L is selected from any of those in Table B below.

In some embodiments, a provided compound or pharmaceutically acceptable salt thereof, is selected from those wherein CBM is

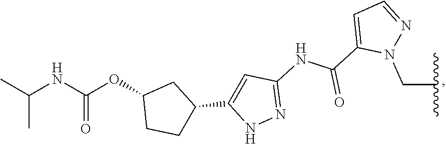 [SMILES: *Cn1nccc1C(=O)Nc1cc([C@@H]2CC[C@H](OC(=O)NC(C)C)C2)[nH]n1]

LBM is selected from any of those in Table A below, and L is selected from any of those in Table B below.

In some embodiments, a provided compound or pharmaceutically acceptable salt thereof, is selected from those wherein CBM is

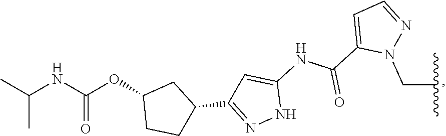 [SMILES: *Cn1nccc1C(=O)Nc1cc([C@@H]2CC[C@H](OC(=O)NC(C)C)C2)n[nH]1]

LBM is selected from any of those in Table A below, and L is selected from any of those in Table B below.

In some embodiments, a provided compound or pharmaceutically acceptable salt thereof, is selected from those wherein CBM is

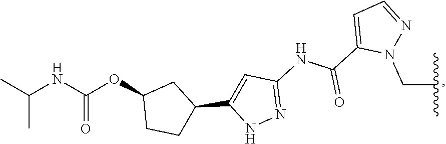 [SMILES: *Cn1nccc1C(=O)Nc1cc([C@H]2CC[C@@H](OC(=O)NC(C)C)C2)[nH]n1]

LBM is selected from any of those in Table A below, and L is selected from any of those in Table B below.

In some embodiments, a provided compound or pharmaceutically acceptable salt thereof, is selected from those wherein CBM is

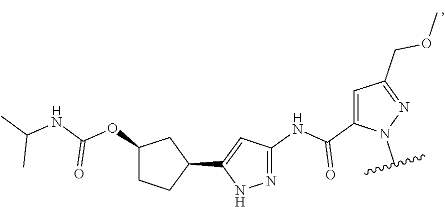 [SMILES: *n1nc(COC)cc1C(=O)Nc1cc([C@H]2CC[C@@H](OC(=O)NC(C)C)C2)[nH]n1]

LBM is selected from any of those in Table A below, and L is selected from any of those in Table B below.

In some embodiments, a provided compound or pharmaceutically acceptable salt thereof, is selected from those wherein CBM is

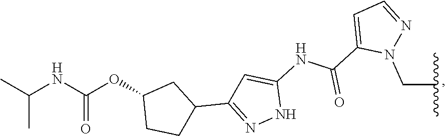 [SMILES: *Cn1nccc1C(=O)Nc1cc(C2CC[C@H](OC(=O)NC(C)C)C2)n[nH]1]

LBM is selected from any of those in Table A below, and L is selected from any of those in Table B below.

In some embodiments, a provided compound or pharmaceutically acceptable salt thereof, is selected from those wherein CBM is

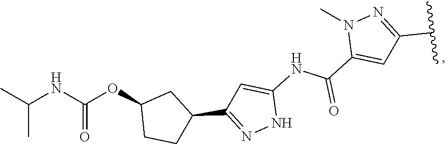 [SMILES: *c1cc(C(=O)Nc2cc([C@H]3CC[C@@H](OC(=O)NC(C)C)C3)n[nH]2)n(C)n1]

LBM is selected from any of those in Table A below, and L is selected from any of those in Table B below.

In some embodiments, a provided compound or pharmaceutically acceptable salt thereof, is selected from those wherein CBM is

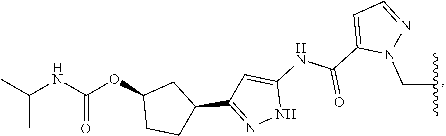 [SMILES: *Cn1nccc1C(=O)Nc1cc([C@H]2CC[C@@H](OC(=O)NC(C)C)C2)n[nH]1]

LBM is selected from any of those in Table A below, and L is selected from any of those in Table B below.

In some embodiments, a provided compound or pharmaceutically acceptable salt thereof, is selected from those wherein CBM is

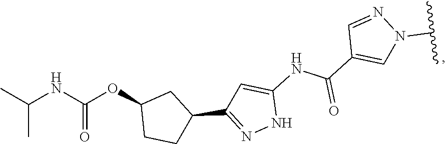 [SMILES: *n1cc(C(=O)Nc2cc([C@H]3CC[C@@H](OC(=O)NC(C)C)C3)n[nH]2)cn1]

LBM is selected from any of those in Table A below, and L is selected from any of those in Table B below.

In some embodiments, a provided compound or pharmaceutically acceptable salt thereof, is selected from those wherein CBM is

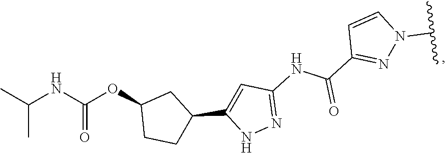 [SMILES: *n1ccc(C(=O)Nc2cc([C@H]3CC[C@@H](OC(=O)NC(C)C)C3)[nH]n2)n1]

LBM is selected from any of those in Table A below, and L is selected from any of those in Table B below.

In some embodiments, a provided compound or pharmaceutically acceptable salt thereof, is selected from those wherein CBM is

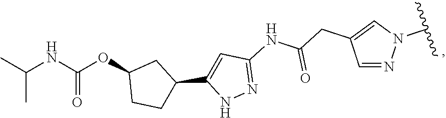 [SMILES: *n1cc(CC(=O)Nc2cc([C@H]3CC[C@@H](OC(=O)NC(C)C)C3)[nH]n2)cn1]

LBM is selected from any of those in Table A below, and L is selected from any of those in Table B below.

In some embodiments, a provided compound or pharmaceutically acceptable salt thereof, is selected from those wherein CBM is

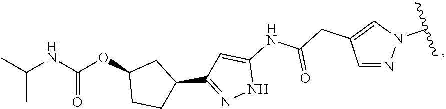 [SMILES: *n1cc(CC(=O)Nc2cc([C@H]3CC[C@@H](OC(=O)NC(C)C)C3)n[nH]2)cn1]

LBM is selected from any of those in Table A below, and L is selected from any of those in Table B below.

In some embodiments, a provided compound or pharmaceutically acceptable salt thereof, is selected from those wherein CBM is

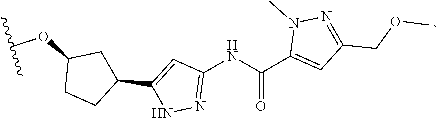 [SMILES: *O[C@@H]1CC[C@H](c2cc(NC(=O)c3cc(COC)nn3C)n[nH]2)C1]

LBM is selected from any of those in Table A below, and L is selected from any of those in Table B below.

In some embodiments, a provided compound or pharmaceutically acceptable salt thereof, is selected from those wherein CBM is

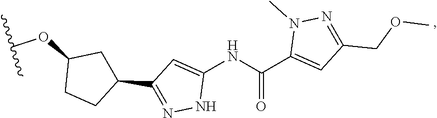 [SMILES: *O[C@@H]1CC[C@H](c2cc(NC(=O)c3cc(COC)nn3C)[nH]n2)C1]

LBM is selected from any of those in Table A below, and L is selected from any of those in Table B below.

TABLE A
Exemplified E3 Ligase Binding Moiety (LBM)

| | |
|---|---|
| 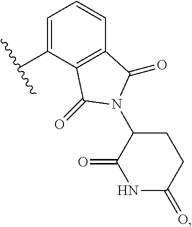 [SMILES: *c1cccc2c1C(=O)N(C1CCC(=O)NC1=O)C2=O] | (a) |
| 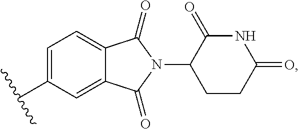 [SMILES: *c1ccc2c(c1)C(=O)N(C1CCC(=O)NC1=O)C2=O] | (b) |
| 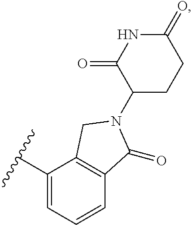 [SMILES: *c1cccc2c1CN(C1CCC(=O)NC1=O)C2=O] | (c) |
| 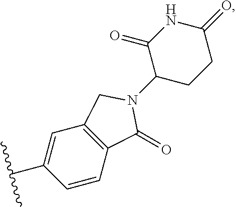 [SMILES: *c1ccc2c(c1)CN(C1CCC(=O)NC1=O)C2=O] | (d) |
| 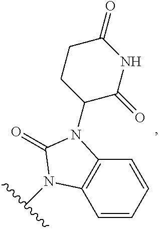 [SMILES: *n1c(=O)n(C2CCC(=O)NC2=O)c2ccccc21] | (e) |
| 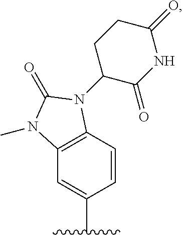 [SMILES: *c1ccc2c(c1)n(C)c(=O)n2C1CCC(=O)NC1=O] | (f) |
| 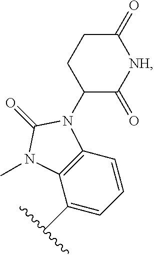 [SMILES: *c1cccc2c1n(C)c(=O)n2C1CCC(=O)NC1=O] | (g) |
| 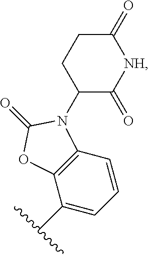 [SMILES: *c1cccc2c1oc(=O)n2C1CCC(=O)NC1=O] | (h) |
| 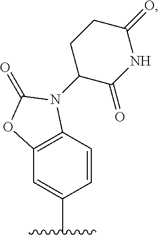 [SMILES: *c1ccc2c(c1)oc(=O)n2C1CCC(=O)NC1=O] | (i) |
| 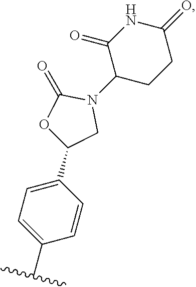 [SMILES: *c1ccc([C@H]2CN(C3CCC(=O)NC3=O)C(=O)O2)cc1] | (j) |
| 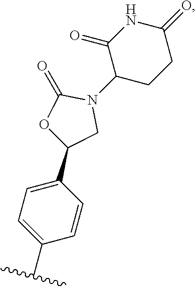 [SMILES: *c1ccc([C@@H]2CN(C3CCC(=O)NC3=O)C(=O)O2)cc1] | (k) |
| 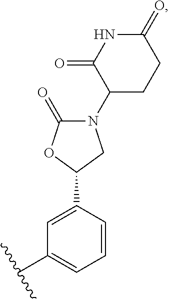 [SMILES: *c1cccc([C@H]2CN(C3CCC(=O)NC3=O)C(=O)O2)c1] | (l) |
| 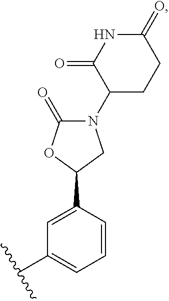 [SMILES: *c1cccc([C@@H]2CN(C3CCC(=O)NC3=O)C(=O)O2)c1] | (m) |
| 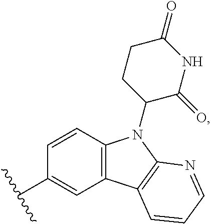 [SMILES: *c1ccc2c(c1)c1cccnc1n2C1CCC(=O)NC1=O] | (n) |
| 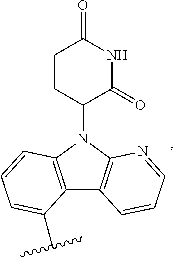 [SMILES: *c1cccc2c1c1cccnc1n2C1CCC(=O)NC1=O] | (o) |
| 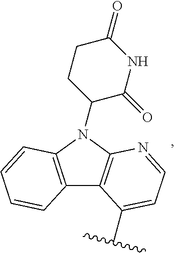 [SMILES: *c1ccnc2c1c1ccccc1n2C1CCC(=O)NC1=O] | (p) |
| 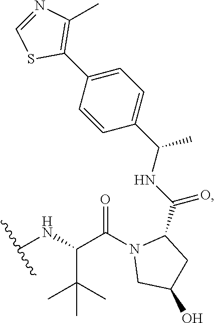 [SMILES: *N[C@H](C(=O)N1C[C@H](O)C[C@H]1C(=O)N[C@@H](C)c1ccc(-c2scnc2C)cc1)C(C)(C)C] | (q) |
| 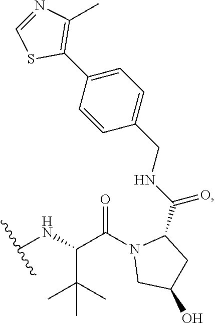 [SMILES: *N[C@H](C(=O)N1C[C@H](O)C[C@H]1C(=O)NCc1ccc(-c2scnc2C)cc1)C(C)(C)C] | (r) |
| 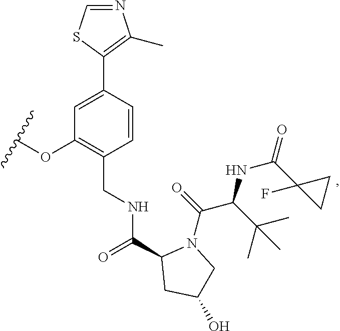 [SMILES: *Oc1cc(-c2scnc2C)ccc1CNC(=O)[C@@H]1C[C@@H](O)CN1C(=O)[C@@H](NC(=O)C1(F)CC1)C(C)(C)C] | (s) |
| 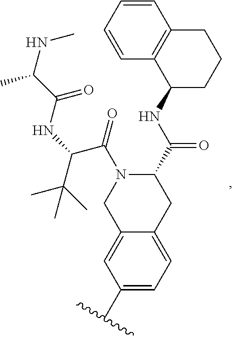 [SMILES: *c1ccc2c(c1)CN(C(=O)[C@@H](NC(=O)[C@H](C)NC)C(C)(C)C)[C@H](C(=O)N[C@@H]1CCCc3ccccc31)C2] | (t) |
| 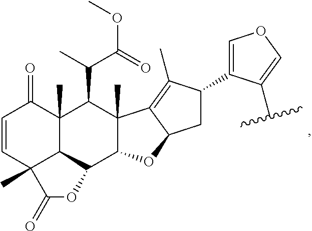 [SMILES: *c1cocc1[C@@H]1C[C@H]2O[C@@H]3[C@@H]4OC(=O)[C@]5(C)C=CC(=O)[C@](C)([C@@H](C(C)C(=O)OC)[C@]3(C)C2=C1C)[C@@H]45] | (u) |
| 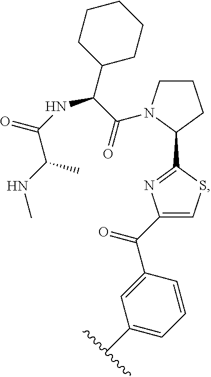 [SMILES: *c1cccc(C(=O)c2csc([C@@H]3CCCN3C(=O)[C@@H](NC(=O)[C@H](C)NC)C3CCCCC3)n2)c1] | (v) |
| 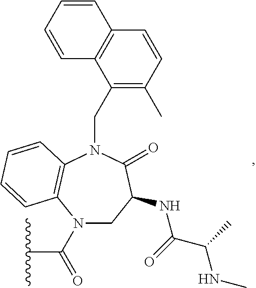 [SMILES: *C(=O)N1C[C@H](NC(=O)[C@H](C)NC)C(=O)N(Cc2c(C)ccc3ccccc23)c2ccccc21] | (w) |
| 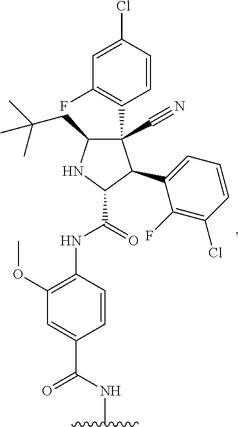 [SMILES: *NC(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1] | (x) |
| 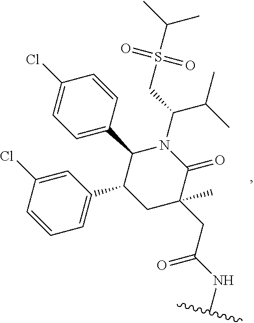 [SMILES: *NC(=O)C[C@@]1(C)C[C@H](c2cccc(Cl)c2)[C@@H](c2ccc(Cl)cc2)N([C@H](CS(=O)(=O)C(C)C)C(C)C)C1=O] | (y) |
| 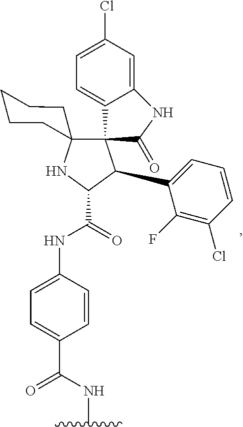 [SMILES: *NC(=O)c1ccc(NC(=O)[C@@H]2NC3(CCCCC3)[C@@]3(C(=O)Nc4cc(Cl)ccc43)[C@H]2c2cccc(Cl)c2F)cc1] | (z) |
| 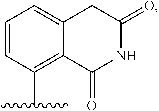 [SMILES: *c1cccc2c1C(=O)NC(=O)C2] | (aa) |
| 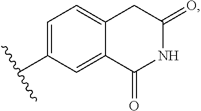 [SMILES: *c1ccc2c(c1)C(=O)NC(=O)C2] | (bb) |
| 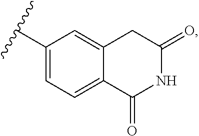 [SMILES: *c1ccc2c(c1)CC(=O)NC2=O] | (cc) |
| 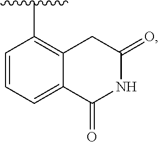 [SMILES: *c1cccc2c1CC(=O)NC2=O] | (dd) |
| 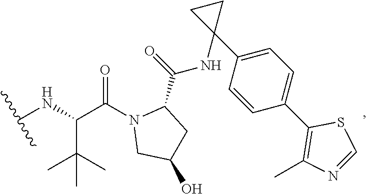 [SMILES: *N[C@H](C(=O)N1C[C@H](O)C[C@H]1C(=O)NC1(c2ccc(-c3scnc3C)cc2)CC1)C(C)(C)C] | (ee) |
| 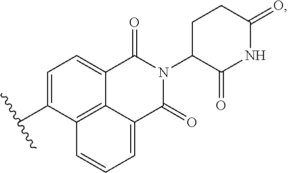 [SMILES: *c1ccc2c3c(cccc13)C(=O)N(C1CCC(=O)NC1=O)C2=O] | (ff) |
| 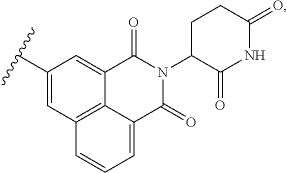 [SMILES: *c1cc2c3c(cccc3c1)C(=O)N(C1CCC(=O)NC1=O)C2=O] | (gg) |
| 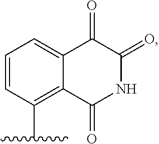 [SMILES: *c1cccc2c1C(=O)NC(=O)C2=O] | (hh) |
| 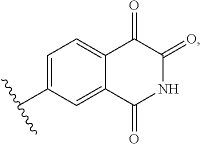 [SMILES: *c1ccc2c(c1)C(=O)NC(=O)C2=O] | (ii) |
| 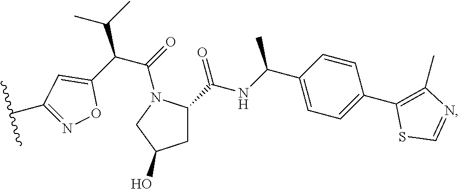 [SMILES: *c1cc([C@H](C(=O)N2C[C@H](O)C[C@H]2C(=O)N[C@@H](C)c2ccc(-c3scnc3C)cc2)C(C)C)on1] | (jj) |
| 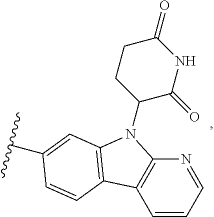 [SMILES: *c1ccc2c3cccnc3n(C3CCC(=O)NC3=O)c2c1] | (kk) |
| 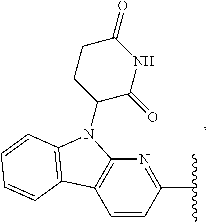 [SMILES: *c1ccc2c3ccccc3n(C3CCC(=O)NC3=O)c2n1] | (ll) |
| 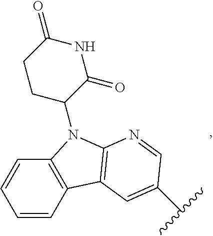 [SMILES: *c1cnc2c(c1)c1ccccc1n2C1CCC(=O)NC1=O] | (mm) |
| 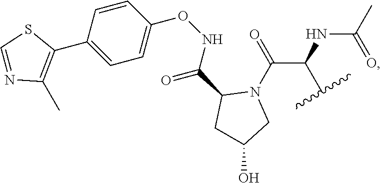 [SMILES: *[C@H](NC(C)=O)C(=O)N1C[C@H](O)C[C@H]1C(=O)NOc1ccc(-c2scnc2C)cc1] | (nn) |
| 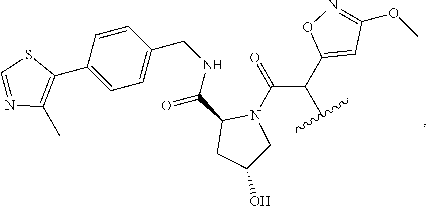 [SMILES: *C(C(=O)N1C[C@H](O)C[C@H]1C(=O)NCc1ccc(-c2scnc2C)cc1)c1cc(OC)no1] | (oo) |
| 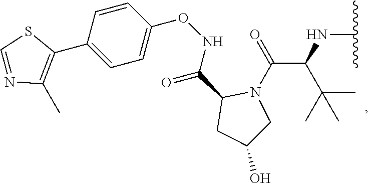 [SMILES: *N[C@H](C(=O)N1C[C@H](O)C[C@H]1C(=O)NOc1ccc(-c2scnc2C)cc1)C(C)(C)C] | (pp) |
| 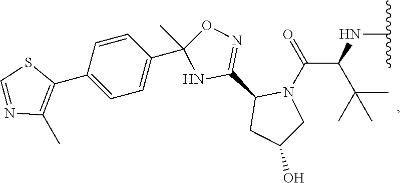 [SMILES: *N[C@H](C(=O)N1C[C@H](O)C[C@H]1C1=NOC(C)(c2ccc(-c3scnc3C)cc2)N1)C(C)(C)C] | (qq) |
| 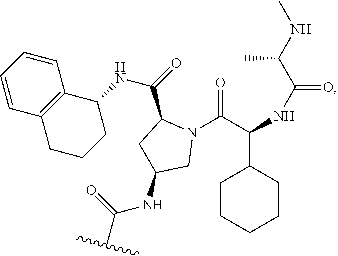 [SMILES: *C(=O)N[C@H]1C[C@@H](C(=O)N[C@@H]2CCCc3ccccc32)N(C(=O)[C@@H](NC(=O)[C@H](C)NC)C2CCCCC2)C1] | (rr) |
| 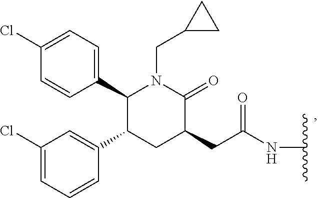 [SMILES: *NC(=O)C[C@H]1C[C@H](c2cccc(Cl)c2)[C@@H](c2ccc(Cl)cc2)N(CC2CC2)C1=O] | (ss) |
| 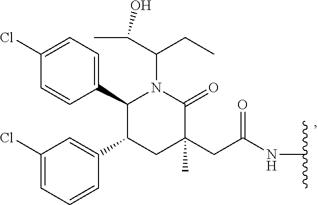 [SMILES: *NC(=O)C[C@@]1(C)C[C@H](c2cccc(Cl)c2)[C@@H](c2ccc(Cl)cc2)N(C(CC)[C@H](C)O)C1=O] | (tt) |
| 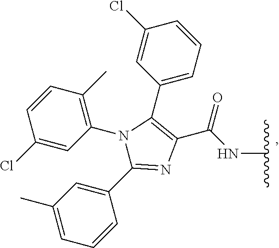 [SMILES: *NC(=O)c1nc(-c2cccc(C)c2)n(-c2cc(Cl)ccc2C)c1-c1cccc(Cl)c1] | (uu) |
| 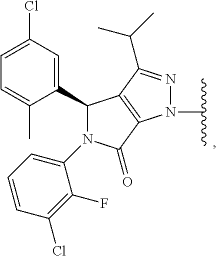 [SMILES: *n1nc(C(C)C)c2c1C(=O)N(c1cccc(Cl)c1F)[C@H]2c1cc(Cl)ccc1C] | (vv) |
| 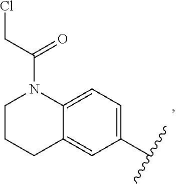 [SMILES: *c1ccc2c(c1)CCCN2C(=O)CCl] | (ww) |
| 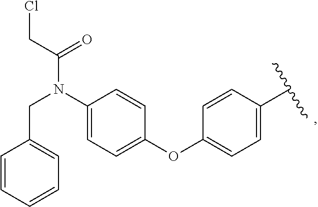 [SMILES: *c1ccc(Oc2ccc(N(Cc3ccccc3)C(=O)CCl)cc2)cc1] | (xx) |
| 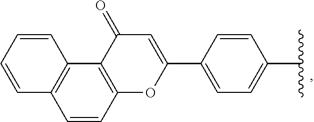 [SMILES: *c1ccc(-c2cc(=O)c3c(ccc4ccccc43)o2)cc1] | (vv) |
| 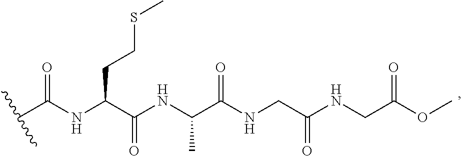 [SMILES: *C(=O)N[C@@H](CCSC)C(=O)N[C@@H](C)C(=O)NCC(=O)NCC(=O)OC] | (zz) |
| 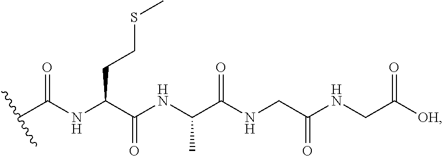 [SMILES: *C(=O)N[C@@H](CCSC)C(=O)N[C@@H](C)C(=O)NCC(=O)NCC(=O)O] | (aaa) |
| 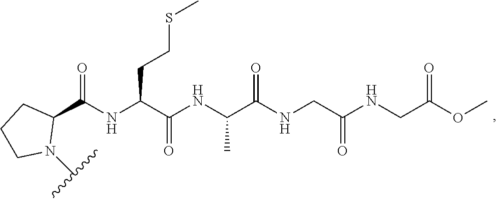 [SMILES: *N1CCC[C@H]1C(=O)N[C@@H](CCSC)C(=O)N[C@@H](C)C(=O)NCC(=O)NCC(=O)OC] | (bbb) |
| 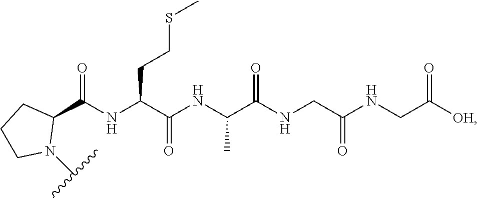 [SMILES: *N1CCC[C@H]1C(=O)N[C@@H](CCSC)C(=O)N[C@@H](C)C(=O)NCC(=O)NCC(=O)O] | (ccc) |
| 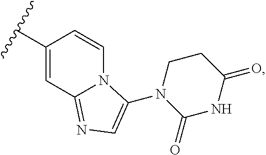 [SMILES: *c1ccn2c(N3CCC(=O)NC3=O)cnc2c1] | (ddd) |
| 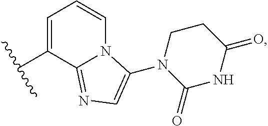 [SMILES: *c1cccn2c(N3CCC(=O)NC3=O)cnc12] | (eee) |
| 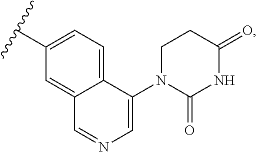 [SMILES: *c1ccc2c(N3CCC(=O)NC3=O)cncc2c1] | (fff) |
| 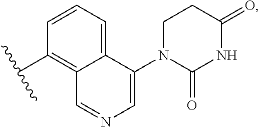 [SMILES: *c1cccc2c(N3CCC(=O)NC3=O)cncc12] | (ggg) |
| 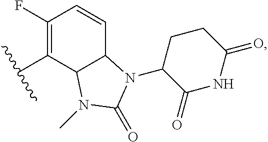 [SMILES: *C1=C(F)C=CC2C1N(C)C(=O)N2C1CCC(=O)NC1=O] | (hhh) |
| 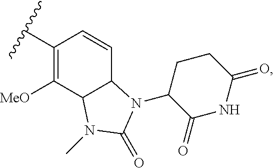 [SMILES: *C1=C(OC)C2C(C=C1)N(C1CCC(=O)NC1=O)C(=O)N2C] | (iii) |
| 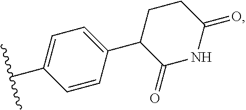 [SMILES: *c1ccc(C2CCC(=O)NC2=O)cc1] | (jjj) |
| 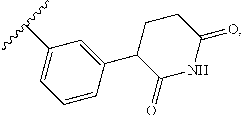 [SMILES: *c1cccc(C2CCC(=O)NC2=O)c1] | (kkk) |
| 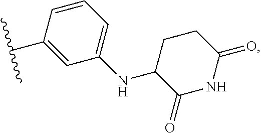 [SMILES: *c1cccc(NC2CCC(=O)NC2=O)c1] | (lll) |
| 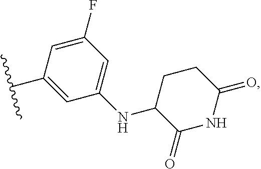 [SMILES: *c1cc(F)cc(NC2CCC(=O)NC2=O)c1] | (mmm) |
| 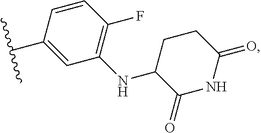 [SMILES: *c1ccc(F)c(NC2CCC(=O)NC2=O)c1] | (nnn) |
| 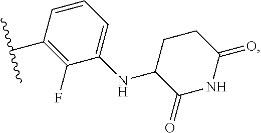 [SMILES: *c1cccc(NC2CCC(=O)NC2=O)c1F] | (ooo) |
| 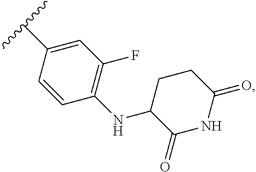 [SMILES: *c1ccc(NC2CCC(=O)NC2=O)c(F)c1] | (ppp) |
| 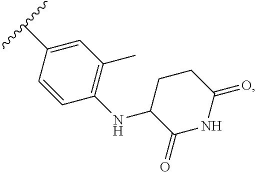 [SMILES: *c1ccc(NC2CCC(=O)NC2=O)c(C)c1] | (qqq) |
| 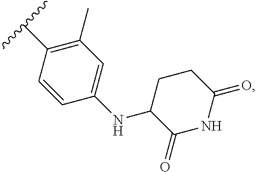 [SMILES: *c1ccc(NC2CCC(=O)NC2=O)cc1C] | (rrr) |
| 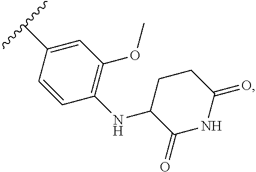 [SMILES: *c1ccc(NC2CCC(=O)NC2=O)c(OC)c1] | (sss) |
| 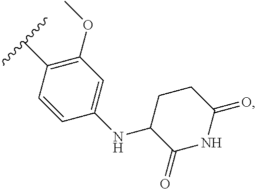 [SMILES: *c1ccc(NC2CCC(=O)NC2=O)cc1OC] | (ttt) |
| 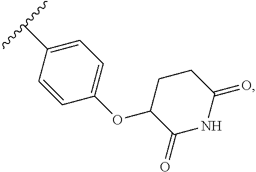 [SMILES: *c1ccc(OC2CCC(=O)NC2=O)cc1] | (uuu) |
| 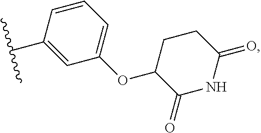 [SMILES: *c1cccc(OC2CCC(=O)NC2=O)c1] | (vvv) |
| 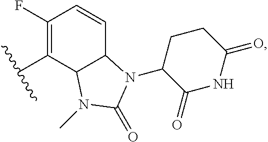 [SMILES: *C1=C(F)C=CC2C1N(C)C(=O)N2C1CCC(=O)NC1=O] | (www) |
| 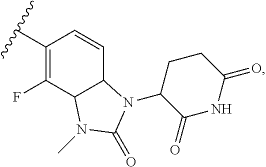 [SMILES: *C1=C(F)C2C(C=C1)N(C1CCC(=O)NC1=O)C(=O)N2C] | (xxx) |
| 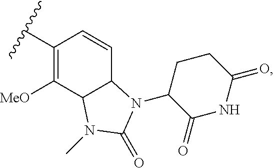 [SMILES: *C1=C(OC)C2C(C=C1)N(C1CCC(=O)NC1=O)C(=O)N2C] | (yyy) |
| 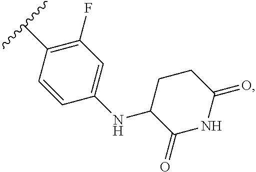 [SMILES: *c1ccc(NC2CCC(=O)NC2=O)cc1F] | (zzz) |
| 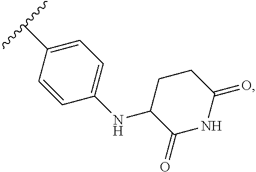 [SMILES: *c1ccc(NC2CCC(=O)NC2=O)cc1] | (aaaa) |
| 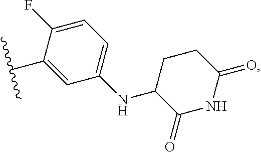 [SMILES: *c1cc(NC2CCC(=O)NC2=O)ccc1F] | (bbbb) |
| 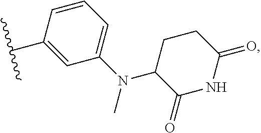 [SMILES: *c1cccc(N(C)C2CCC(=O)NC2=O)c1] | (cccc) |
| 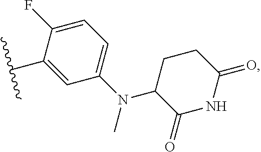 [SMILES: *c1cc(N(C)C2CCC(=O)NC2=O)ccc1F] | (dddd) |
| 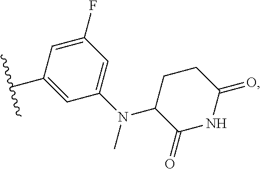 [SMILES: *c1cc(F)cc(N(C)C2CCC(=O)NC2=O)c1] | (eeee) |
| 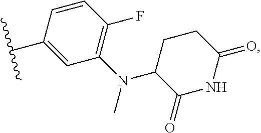 [SMILES: *c1ccc(F)c(N(C)C2CCC(=O)NC2=O)c1] | (ffff) |
| 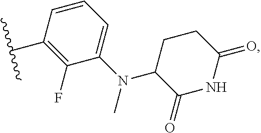 [SMILES: *c1cccc(N(C)C2CCC(=O)NC2=O)c1F] | (gggg) |
| 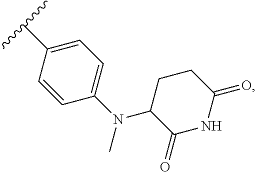 [SMILES: *c1ccc(N(C)C2CCC(=O)NC2=O)cc1] | (hhhh) |
| 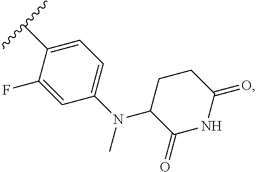 [SMILES: *c1ccc(N(C)C2CCC(=O)NC2=O)cc1F] | (iiii) |
| 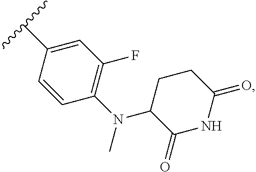 [SMILES: *c1ccc(N(C)C2CCC(=O)NC2=O)c(F)c1] | (jjjj) |
| 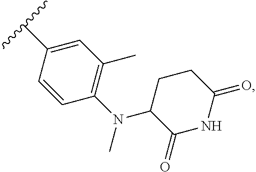 [SMILES: *c1ccc(N(C)C2CCC(=O)NC2=O)c(C)c1] | (kkkk) |
| 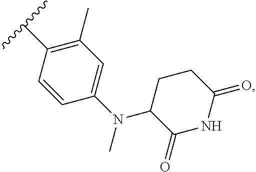 [SMILES: *c1ccc(N(C)C2CCC(=O)NC2=O)cc1C] | (llll) |
| 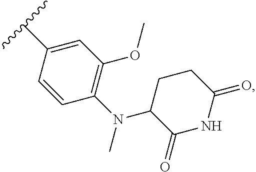 [SMILES: *c1ccc(N(C)C2CCC(=O)NC2=O)c(OC)c1] | (mmmm) |
| 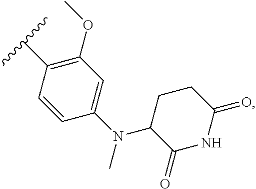 [SMILES: *c1ccc(N(C)C2CCC(=O)NC2=O)cc1OC] | (nnnn) |
| 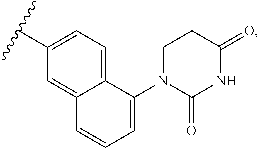 [SMILES: *c1ccc2c(N3CCC(=O)NC3=O)cccc2c1] | (oooo) |
| 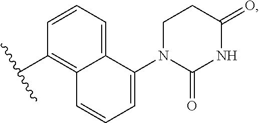 [SMILES: *c1cccc2c(N3CCC(=O)NC3=O)cccc12] | (pppp) |
| 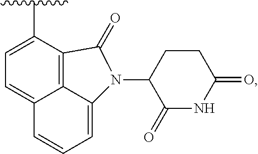 [SMILES: *c1ccc2cccc3c2c1C(=O)N3C1CCC(=O)NC1=O] | (qqqq) |
| 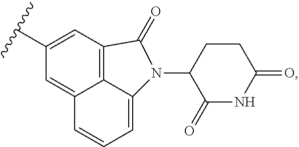 [SMILES: *c1cc2c3c(cccc3c1)N(C1CCC(=O)NC1=O)C2=O] | (rrrr) |
| 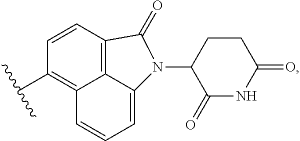 [SMILES: *c1ccc2c3c(cccc13)N(C1CCC(=O)NC1=O)C2=O] | (ssss) |
| 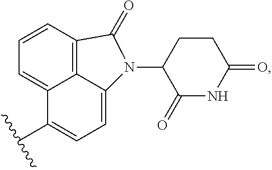 [SMILES: *c1ccc2c3c(cccc13)C(=O)N2C1CCC(=O)NC1=O] | (tttt) |
| 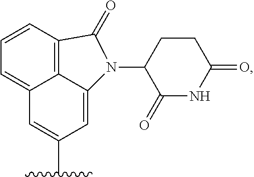 [SMILES: *c1cc2c3c(cccc3c1)C(=O)N2C1CCC(=O)NC1=O] | (uuuu) |
| 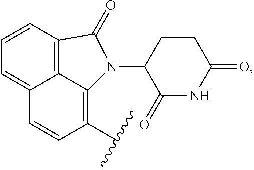 [SMILES: *c1ccc2cccc3c2c1N(C1CCC(=O)NC1=O)C3=O] | (vvvv) |
| 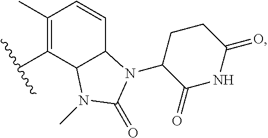 [SMILES: *C1=C(C)C=CC2C1N(C)C(=O)N2C1CCC(=O)NC1=O] | (wwww) |
| 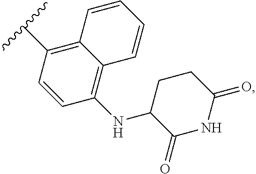 [SMILES: *c1ccc(NC2CCC(=O)NC2=O)c2ccccc12] | (xxxx) |
| 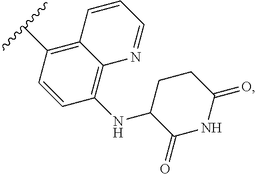 [SMILES: *c1ccc(NC2CCC(=O)NC2=O)c2ncccc12] | (yyyy) |
| 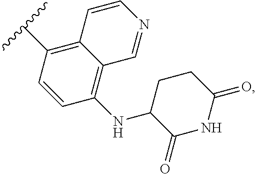 [SMILES: *c1ccc(NC2CCC(=O)NC2=O)c2cnccc12] | (zzzz) |
| 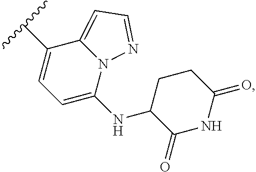 [SMILES: *c1ccc(NC2CCC(=O)NC2=O)n2nccc12] | (aaaaa) |
| 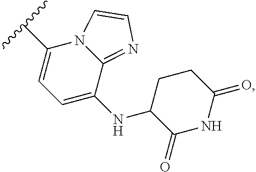 [SMILES: *c1ccc(NC2CCC(=O)NC2=O)c2nccn12] | (bbbbb) |
| 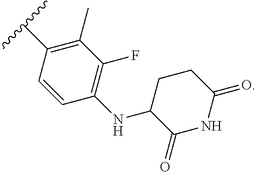 [SMILES: *c1ccc(NC2CCC(=O)NC2=O)c(F)c1C] | (ccccc) |

TABLE B
Exemplified Linkers (L)

| | |
|---|---|
| 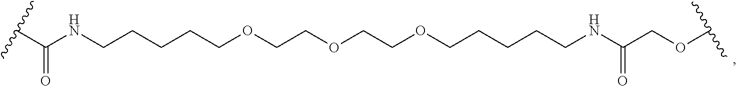 [SMILES: *OCC(=O)NCCCCCOCCOCCOCCCCCNC(*)=O] | (1) |
| 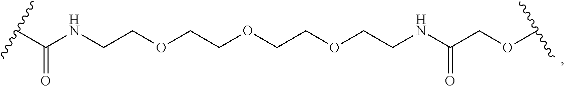 [SMILES: *OCC(=O)NCCOCCOCCOCCNC(*)=O] | (2) |
| 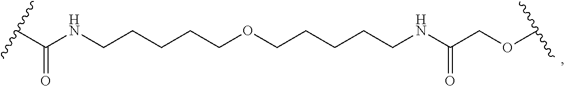 [SMILES: *OCC(=O)NCCCCCOCCCCCNC(*)=O] | (3) |
| 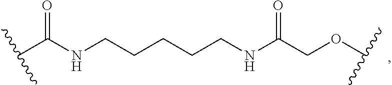 [SMILES: *OCC(=O)NCCCCCNC(*)=O] | (4) |
| 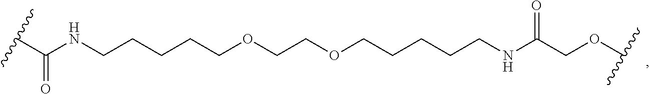 [SMILES: *OCC(=O)NCCCCCOCCOCCCCCNC(*)=O] | (5) |
| 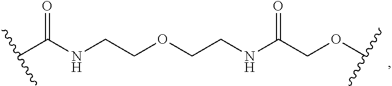 [SMILES: *OCC(=O)NCCOCCNC(*)=O] | (6) |
| 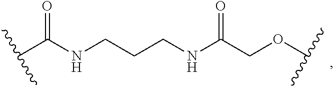 [SMILES: *OCC(=O)NCCCNC(*)=O] | (7) |
| 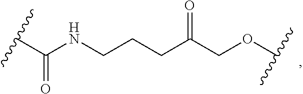 [SMILES: *OCC(=O)CCCNC(*)=O] | (8) |
| 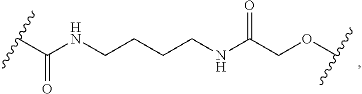 [SMILES: *OCC(=O)NCCCCNC(*)=O] | (9) |
| 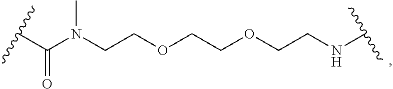 [SMILES: *NCCOCCOCCN(C)C(*)=O] | (10) |
| 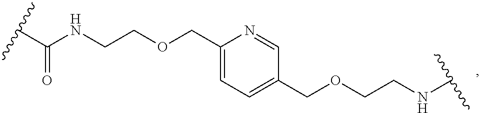 [SMILES: *NCCOCc1ccc(COCCNC(*)=O)nc1] | (11) |
| 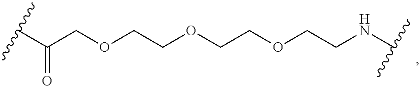 [SMILES: *NCCOCCOCCOCC(*)=O] | (12) |
| 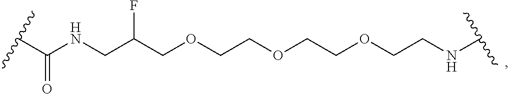 [SMILES: *NCCOCCOCCOCC(F)CNC(*)=O] | (13) |
| 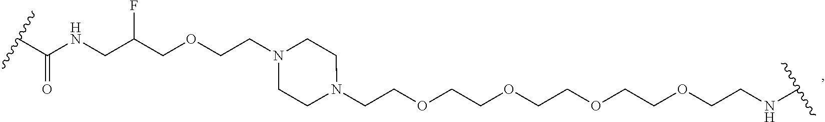 [SMILES: *NCCOCCOCCOCCOCCN1CCN(CCOCC(F)CNC(*)=O)CC1] | (14) |
| 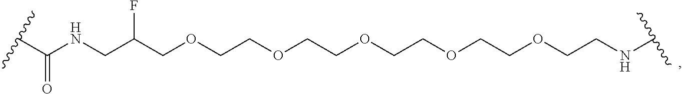 [SMILES: *NCCOCCOCCOCCOCCOCC(F)CNC(*)=O] | (15) |
| 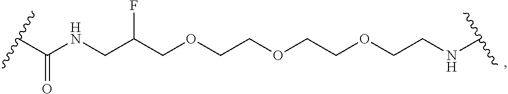 [SMILES: *NCCOCCOCCOCC(F)CNC(*)=O] | (16) |
| 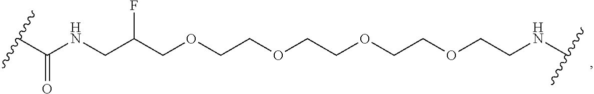 [SMILES: *NCCOCCOCCOCCOCC(F)CNC(*)=O] | (17) |
| 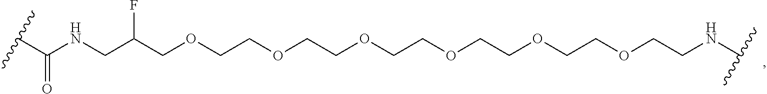 [SMILES: *NCCOCCOCCOCCOCCOCCOCC(F)CNC(*)=O] | (18) |
| 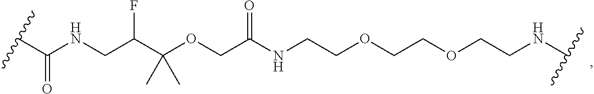 [SMILES: *NCCOCCOCCNC(=O)COC(C)(C)C(F)CNC(*)=O] | (19) |
| 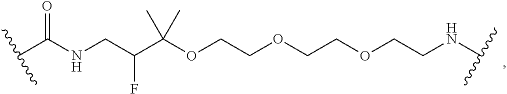 [SMILES: *NCCOCCOCCOC(C)(C)C(F)CNC(*)=O] | (20) |
| 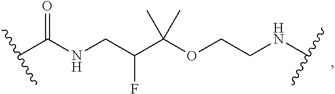 [SMILES: *NCCOC(C)(C)C(F)CNC(*)=O] | (21) |
| 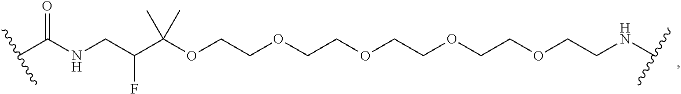 [SMILES: *NCCOCCOCCOCCOCCOC(C)(C)C(F)CNC(*)=O] | (22) |
| 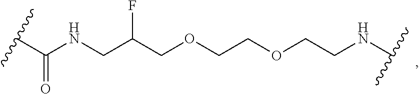 [SMILES: *NCCOCCOCC(F)CNC(*)=O] | (23) |
| 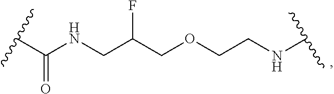 [SMILES: *NCCOCC(F)CNC(*)=O] | (24) |
| 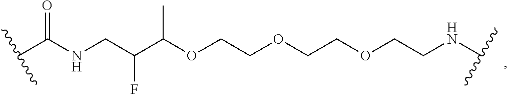 [SMILES: *NCCOCCOCCOC(C)C(F)CNC(*)=O] | (25) |
| 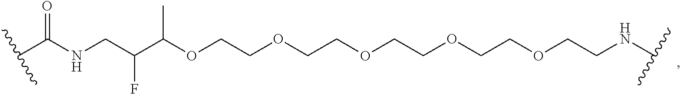 [SMILES: *NCCOCCOCCOCCOCCOC(C)C(F)CNC(*)=O] | (26) |
| 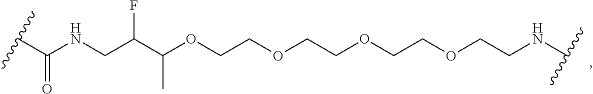 [SMILES: *NCCOCCOCCOCCOC(C)C(F)CNC(*)=O] | (27) |
| 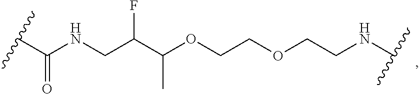 [SMILES: *NCCOCCOC(C)C(F)CNC(*)=O] | (28) |
| 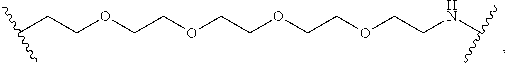 [SMILES: *CCOCCOCCOCCOCCN*] | (29) |
| 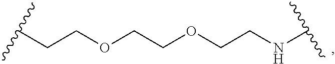 [SMILES: *CCOCCOCCN*] | (30) |
| 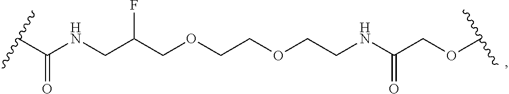 [SMILES: *OCC(=O)NCCOCCOCC(F)CNC(*)=O] | (31) |
| 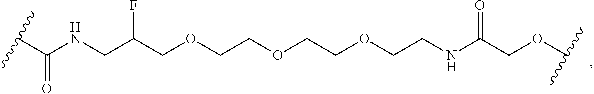 [SMILES: *OCC(=O)NCCOCCOCCOCC(F)CNC(*)=O] | (32) |
| 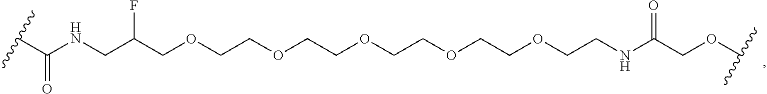 [SMILES: *OCC(=O)NCCOCCOCCOCCOCCOCC(F)CNC(*)=O] | (33) |
| 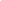 | (34) |
| 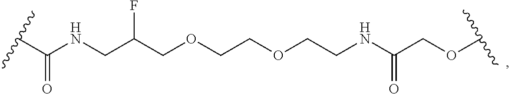 [SMILES: *OCC(=O)NCCOCCOCC(F)CNC(*)=O] | (35) |
| 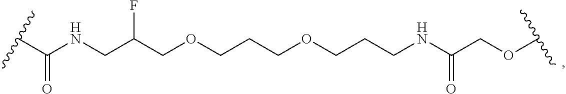 [SMILES: *OCC(=O)NCCCOCCCOCC(F)CNC(*)=O] | (36) |
| 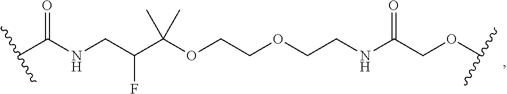 [SMILES: *OCC(=O)NCCOCCOC(C)(C)C(F)CNC(*)=O] | (37) |
| 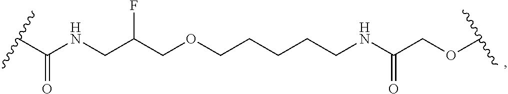 [SMILES: *OCC(=O)NCCCCCOCC(F)CNC(*)=O] | (38) |
| 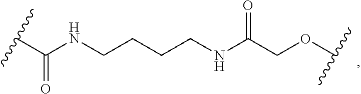 [SMILES: *OCC(=O)NCCCCNC(*)=O] | (39) |
| 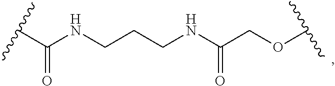 [SMILES: *OCC(=O)NCCCNC(*)=O] | (40) |
| 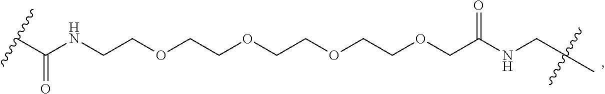 [SMILES: *CNC(=O)COCCOCCOCCOCCNC(*)=O] | (41) |
| 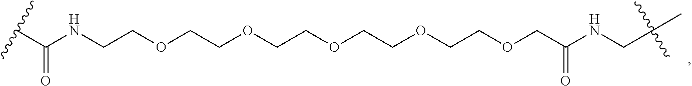 [SMILES: *CNC(=O)COCCOCCOCCOCCOCCNC(*)=O] | (42) |
| 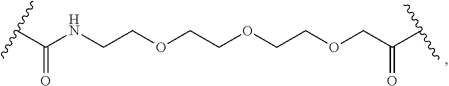 [SMILES: *C(=O)COCCOCCOCCNC(*)=O] | (43) |
| 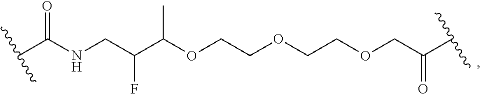 [SMILES: *C(=O)COCCOCCOC(C)C(F)CNC(*)=O] | (44) |
| 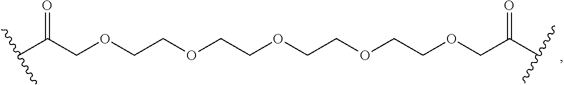 [SMILES: *C(=O)COCCOCCOCCOCCOCC(*)=O] | (45) |
| 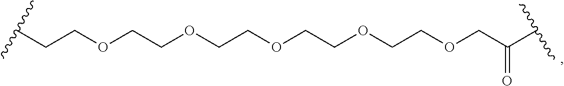 [SMILES: *CCOCCOCCOCCOCCOCC(*)=O] | (46) |
| 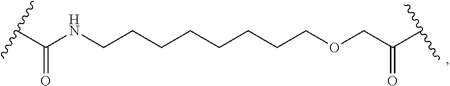 [SMILES: *C(=O)COCCCCCCCCNC(*)=O] | (47) |
| 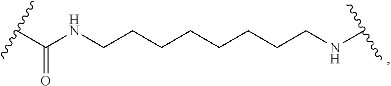 [SMILES: *NCCCCCCCCNC(*)=O] | (49) |
| 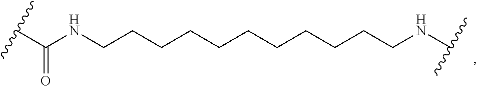 [SMILES: *NCCCCCCCCCCCNC(*)=O] | (50) |
| 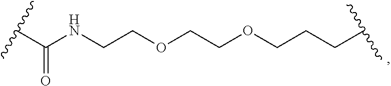 [SMILES: *CCCOCCOCCNC(*)=O] | (51) |
| 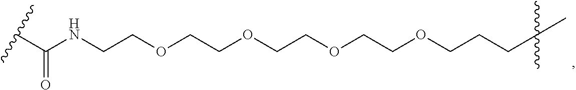 [SMILES: *CCCOCCOCCOCCOCCNC(*)=O] | (52) |
| 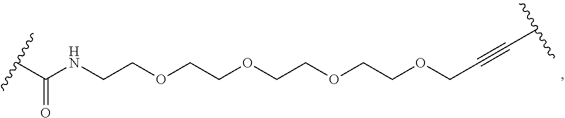 [SMILES: *C#CCOCCOCCOCCOCCNC(*)=O] | (53) |
| 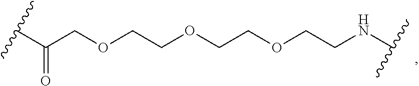 [SMILES: *NCCOCCOCCOCC(*)=O] | (54) |
| 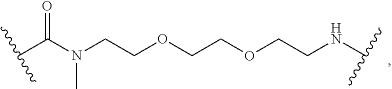 [SMILES: *NCCOCCOCCN(C)C(*)=O] | (55) |
| 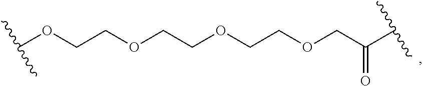 [SMILES: *OCCOCCOCCOCC(*)=O] | (56) |
| 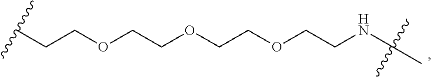 [SMILES: *CCOCCOCCOCCN*] | (57) |
| 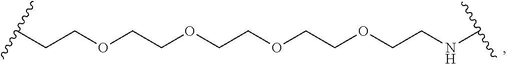 [SMILES: *CCOCCOCCOCCOCCN*] | (58) |
| 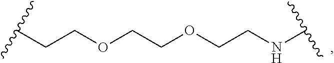 [SMILES: *CCOCCOCCN*] | (59) |
| 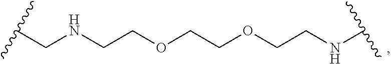 [SMILES: *CNCCOCCOCCN*] | (60) |
| 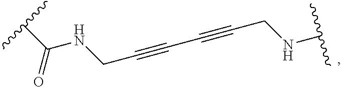 [SMILES: *NCC#CC#CCNC(*)=O] | (61) |
| 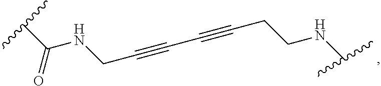 [SMILES: *NCCC#CC#CCNC(*)=O] | (62) |
| 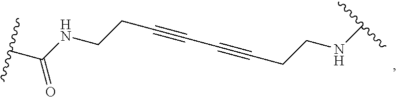 [SMILES: *NCCC#CC#CCCNC(*)=O] | (63) |
| 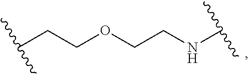 [SMILES: *CCOCCN*] | (64) |
| 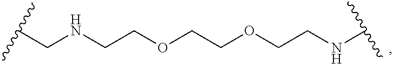 [SMILES: *CNCCOCCOCCN*] | (65) |
| 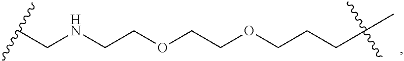 [SMILES: *CCCOCCOCCNC*] | (66) |
| 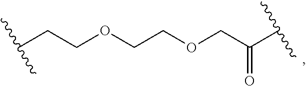 [SMILES: *CCOCCOCC(*)=O] | (67) |
| 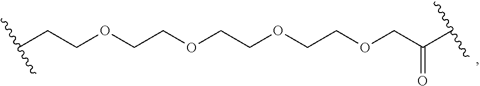 [SMILES: *CCOCCOCCOCCOCC(*)=O] | (68) |
| 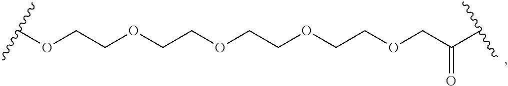 [SMILES: *OCCOCCOCCOCCOCC(*)=O] | (69) |
| 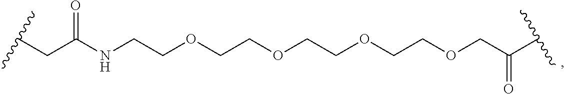 [SMILES: *CC(=O)NCCOCCOCCOCCOCC(*)=O] | (70) |
| 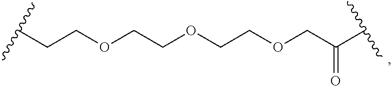 [SMILES: *CCOCCOCCOCC(*)=O] | (71) |
| 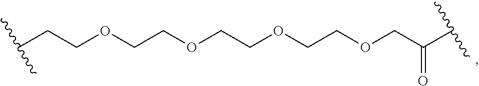 [SMILES: *CCOCCOCCOCCOCC(*)=O] | (72) |
| 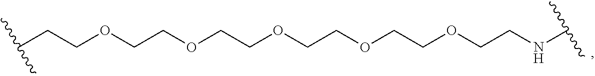 [SMILES: *CCOCCOCCOCCOCCOCCN*] | (73) |
| 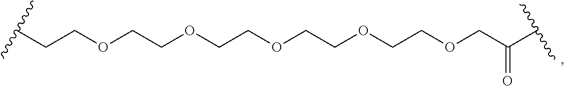 [SMILES: *CCOCCOCCOCCOCCOCC(*)=O] | (74) |
| 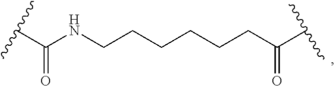 [SMILES: *C(=O)CCCCCCNC(*)=O] | (75) |
| 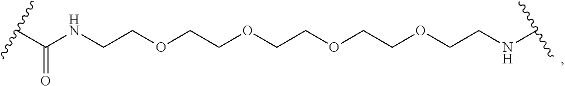 [SMILES: *NCCOCCOCCOCCOCCNC(*)=O] | (76) |
| 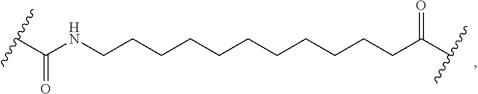 [SMILES: *C(=O)CCCCCCCCCCCNC(*)=O] | (77) |
| 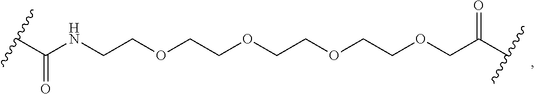 [SMILES: *C(=O)COCCOCCOCCOCCNC(*)=O] | (78) |
| 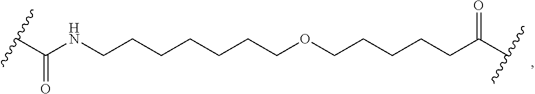 [SMILES: *C(=O)CCCCCOCCCCCCCNC(*)=O] | (79) |
| 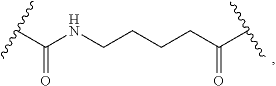 [SMILES: *C(=O)CCCCNC(*)=O] | (80) |
| 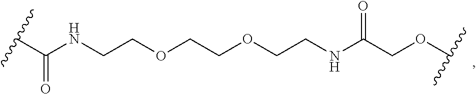 [SMILES: *OCC(=O)NCCOCCOCCNC(*)=O] | (81) |
| 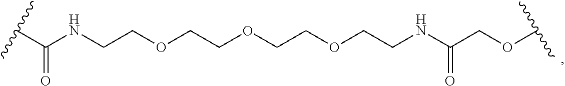 [SMILES: *OCC(=O)NCCOCCOCCOCCNC(*)=O] | (82) |
| 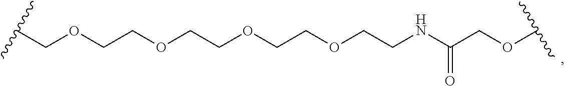 [SMILES: *COCCOCCOCCOCCNC(=O)CO*] | (83) |
| 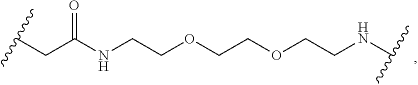 [SMILES: *CC(=O)NCCOCCOCCN*] | (84) |
| 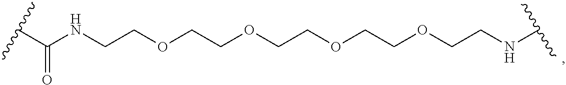 [SMILES: *NCCOCCOCCOCCOCCNC(*)=O] | (85) |
| 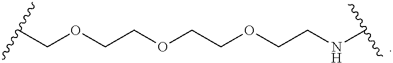 [SMILES: *COCCOCCOCCN*] | (86) |
| 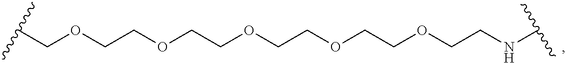 [SMILES: *COCCOCCOCCOCCOCCN*] | (87) |
| 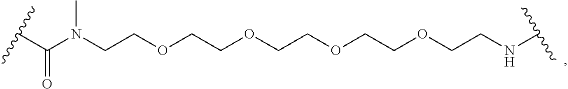 [SMILES: *NCCOCCOCCOCCOCCN(C)C(*)=O] | (88) |
| 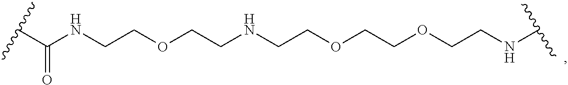 [SMILES: *NCCOCCOCCNCCOCCNC(*)=O] | (89) |
| 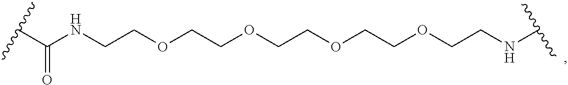 [SMILES: *NCCOCCOCCOCCOCCNC(*)=O] | (90) |
| 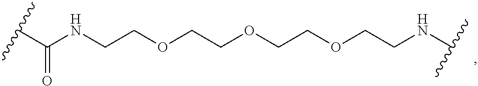 [SMILES: *NCCOCCOCCOCCNC(*)=O] | (91) |
| 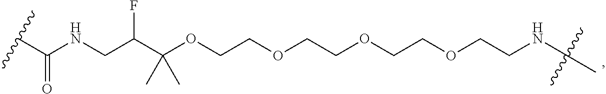 [SMILES: *NCCOCCOCCOCCOC(C)(C)C(F)CNC(*)=O] | (92) |
| 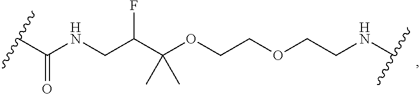 [SMILES: *NCCOCCOC(C)(C)C(F)CNC(*)=O] | (93) |
| 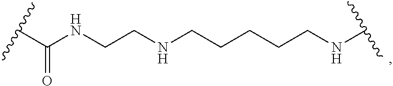 [SMILES: *NCCCCCNCCNC(*)=O] | (94) |
| 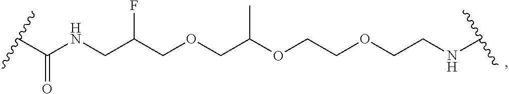 [SMILES: *NCCOCCOC(C)COCC(F)CNC(*)=O] | (95) |
| 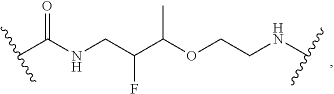 [SMILES: *NCCOC(C)C(F)CNC(*)=O] | (96) |
| 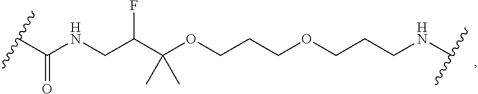 [SMILES: *NCCCOCCCOC(C)(C)C(F)CNC(*)=O] | (97) |
| 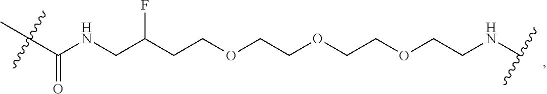 [SMILES: *NCCOCCOCCOCCC(F)CNC(*)=O] | (98) |
| 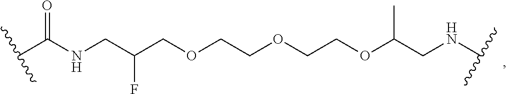 [SMILES: *NCC(C)OCCOCCOCC(F)CNC(*)=O] | (99) |
| 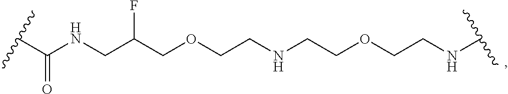 [SMILES: *NCCOCCNCCOCC(F)CNC(*)=O] | (100) |
| 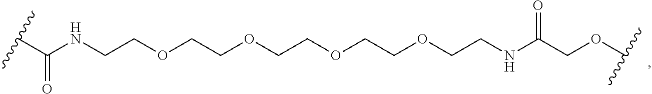 [SMILES: *OCC(=O)NCCOCCOCCOCCOCCNC(*)=O] | (101) |
| 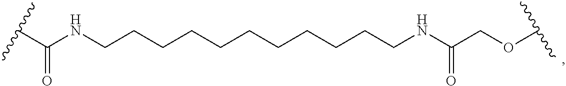 [SMILES: *OCC(=O)NCCCCCCCCCCCNC(*)=O] | (102) |
| 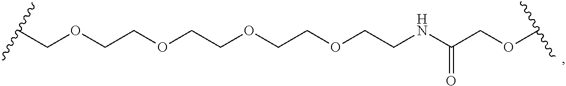 [SMILES: *COCCOCCOCCOCCNC(=O)CO*] | (103) |
| 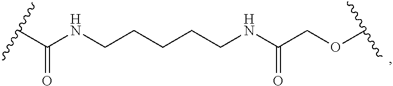 [SMILES: *OCC(=O)NCCCCCNC(*)=O] | (104) |
| 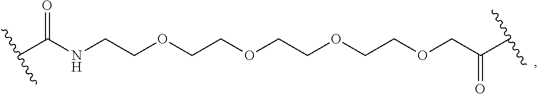 [SMILES: *C(=O)COCCOCCOCCOCCNC(*)=O] | (105) |
| 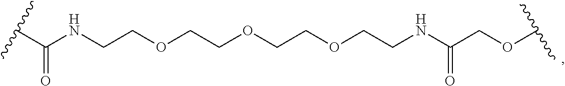 [SMILES: *OCC(=O)NCCOCCOCCOCCNC(*)=O] | (106) |
| 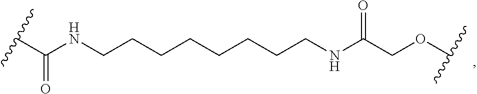 [SMILES: *OCC(=O)NCCCCCCCCNC(*)=O] | (107) |
| 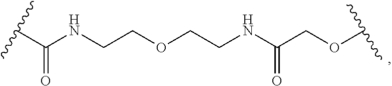 [SMILES: *OCC(=O)NCCOCCNC(*)=O] | (108) |
| 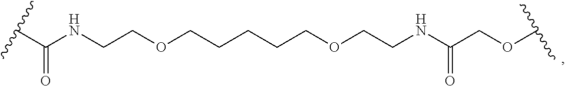 [SMILES: *OCC(=O)NCCOCCCCCOCCNC(*)=O] | (109) |
| 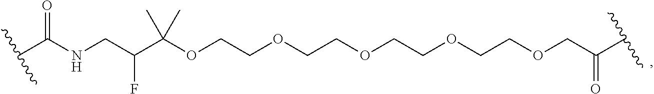 [SMILES: *C(=O)COCCOCCOCCOCCOC(C)(C)C(F)CNC(*)=O] | (110) |
| 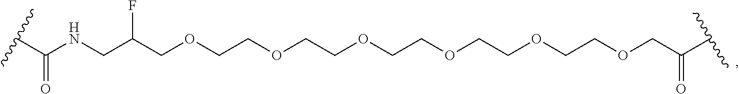 [SMILES: *C(=O)COCCOCCOCCOCCOCCOCC(F)CNC(*)=O] | (111) |
| 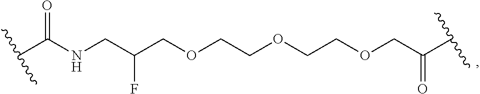 [SMILES: *C(=O)COCCOCCOCC(F)CNC(*)=O] | (112) |
| 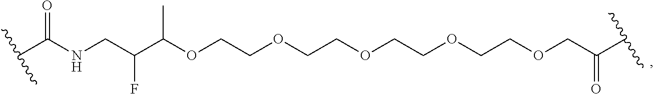 [SMILES: *C(=O)COCCOCCOCCOCCOC(C)C(F)CNC(*)=O] | (113) |
| 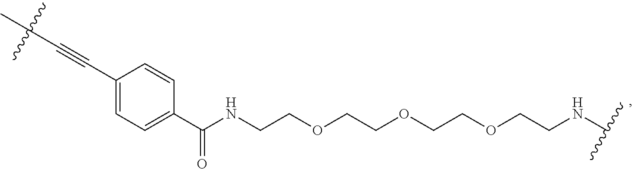 [SMILES: *C#Cc1ccc(C(=O)NCCOCCOCCOCCN*)cc1] | (114) |
| 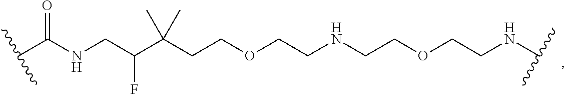 [SMILES: *NCCOCCNCCOCCC(C)(C)C(F)CNC(*)=O] | (115) |
| 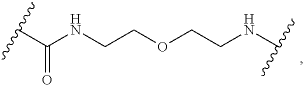 [SMILES: *NCCOCCNC(*)=O] | (116) |
| 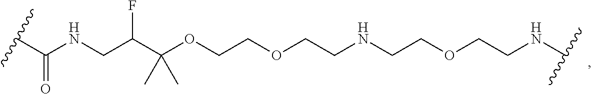 [SMILES: *NCCOCCNCCOCCOC(C)(C)C(F)CNC(*)=O] | (117) |
| 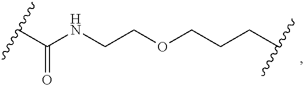 [SMILES: *CCCOCCNC(*)=O] | (118) |
| 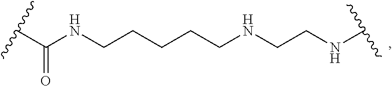 [SMILES: *NCCNCCCCCNC(*)=O] | |
| 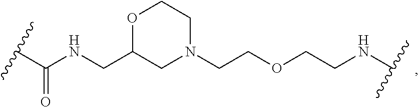 [SMILES: *NCCOCCN1CCOC(CNC(*)=O)C1] | (119) |
| 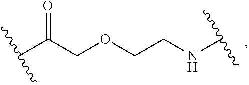 [SMILES: *NCCOCC(*)=O] | |
| 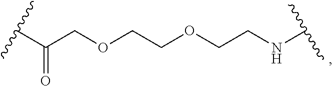 [SMILES: *NCCOCCOCC(*)=O] | (120) |
| 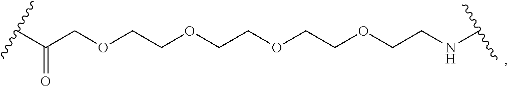 [SMILES: *NCCOCCOCCOCCOCC(*)=O] | (121) |
| 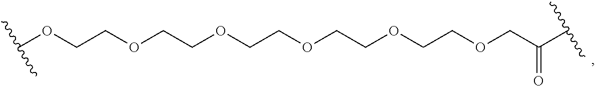 [SMILES: *OCCOCCOCCOCCOCCOCC(*)=O] | (122) |
| 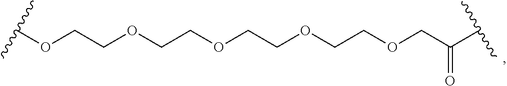 [SMILES: *OCCOCCOCCOCCOCC(*)=O] | (123) |
| 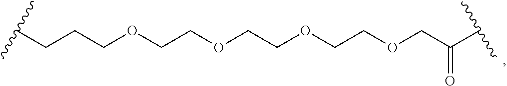 [SMILES: *CCCOCCOCCOCCOCC(*)=O] | (124) |
| 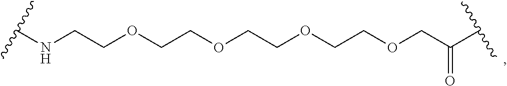 [SMILES: *NCCOCCOCCOCCOCC(*)=O] | (125) |
| 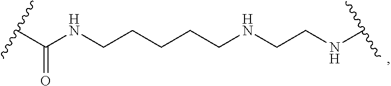 [SMILES: *NCCNCCCCCNC(*)=O] | (126) |
| 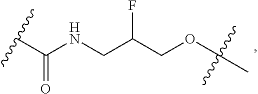 [SMILES: *OCC(F)CNC(*)=O] | |
| 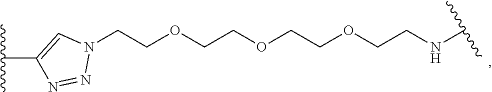 [SMILES: *NCCOCCOCCOCCn1cc(*)nn1] | (127) |
| 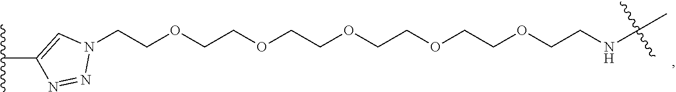 [SMILES: *NCCOCCOCCOCCOCCOCCn1cc(*)nn1] | (128) |
| 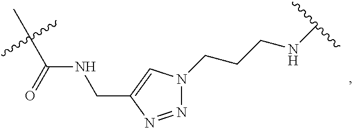 [SMILES: *NCCCn1cc(CNC(*)=O)nn1] | (129) |
| 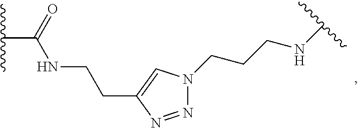 [SMILES: *NCCCn1cc(CCNC(*)=O)nn1] | (130) |
| 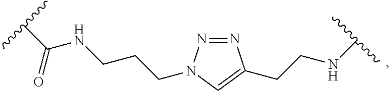 [SMILES: *NCCc1cn(CCCNC(*)=O)nn1] | (131) |
| 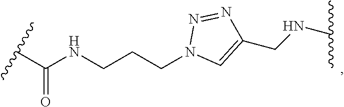 [SMILES: *NCc1cn(CCCNC(*)=O)nn1] | (132) |
| 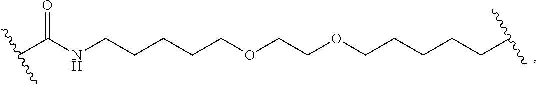 [SMILES: *CCCCCOCCOCCCCCNC(*)=O] | (133) |
| 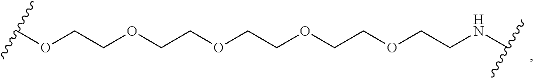 [SMILES: *NCCOCCOCCOCCOCCO*] | (134) |
| 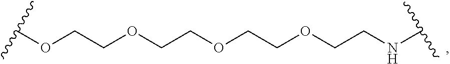 [SMILES: *NCCOCCOCCOCCO*] | (135) |
| 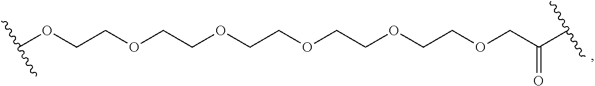 [SMILES: *OCCOCCOCCOCCOCCOCC(*)=O] | (136) |
| 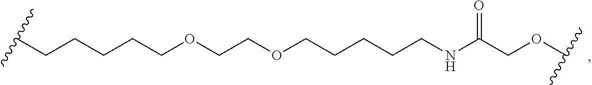 [SMILES: *CCCCCOCCOCCCCCNC(=O)CO*] | (137) |
| 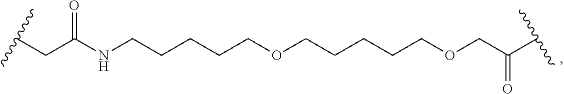 [SMILES: *CC(=O)NCCCCCOCCCCCOCC(*)=O] | (138) |
| 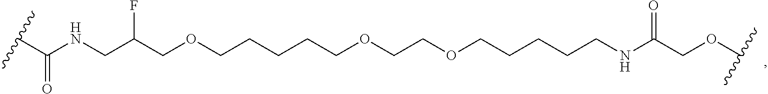 [SMILES: *OCC(=O)NCCCCCOCCOCCCCCOCC(F)CNC(*)=O] | (139) |
| 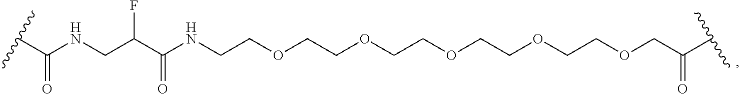 [SMILES: *C(=O)COCCOCCOCCOCCOCCNC(=O)C(F)CNC(*)=O] | (140) |
| 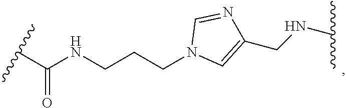 [SMILES: *NCc1cn(CCCNC(*)=O)cn1] | (141) |
| 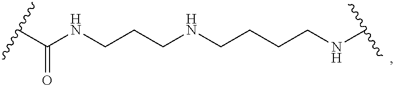 [SMILES: *NCCCCNCCCNC(*)=O] | (142) |
| 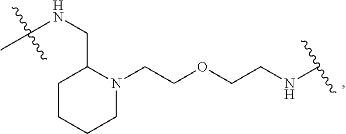 [SMILES: *NCCOCCN1CCCCC1CN*] | (143) |
| 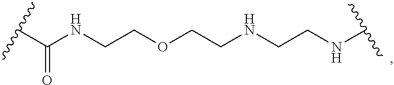 [SMILES: *NCCNCCOCCNC(*)=O] | (144) |
| 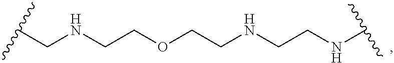 [SMILES: *CNCCOCCNCCN*] | (145) |
| 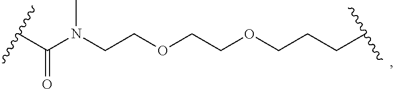 [SMILES: *CCCOCCOCCN(C)C(*)=O] | (146) |
| 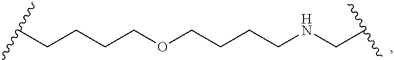 [SMILES: *CCCCOCCCCNC*] | (147) |
| 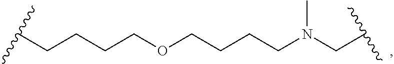 [SMILES: *CCCCOCCCCN(C)C*] | (148) |
| 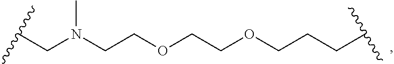 [SMILES: *CCCOCCOCCN(C)C*] | (149) |
| 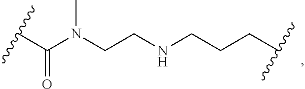 [SMILES: *CCCNCCN(C)C(*)=O] | (150) |
| 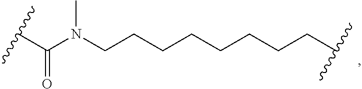 [SMILES: *CCCCCCCCN(C)C(*)=O] | (151) |
| 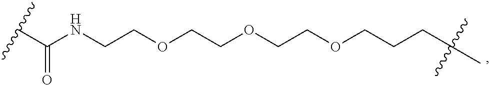 [SMILES: *CCCOCCOCCOCCNC(*)=O] | (152) |
| 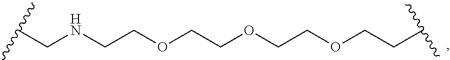 [SMILES: *CCOCCOCCOCCNC*] | (153) |
| 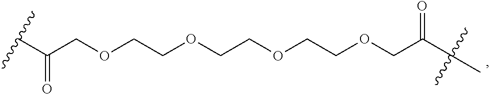 [SMILES: *C(=O)COCCOCCOCCOCC(*)=O] | (154) |
| 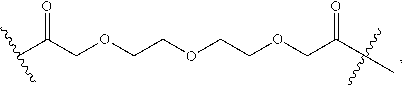 [SMILES: *C(=O)COCCOCCOCC(*)=O] | (155) |
| 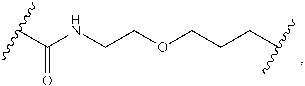 [SMILES: *CCCOCCNC(*)=O] | (156) |
| 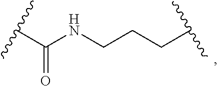 [SMILES: *CCCNC(*)=O] | (157) |
| 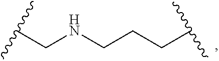 [SMILES: *CCCNC*] | (158) |
| 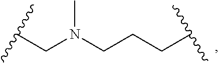 [SMILES: *CCCN(C)C*] | (159) |
| 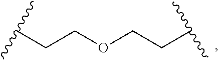 [SMILES: *CCOCC*] | (160) |
| 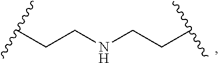 [SMILES: *CCNCC*] | (161) |
| 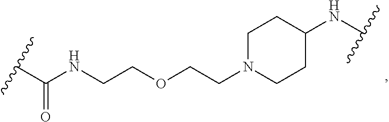 [SMILES: *NC1CCN(CCOCCNC(*)=O)CC1] | (162) |
| 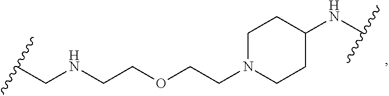 [SMILES: *CNCCOCCN1CCC(N*)CC1] | (163) |
| 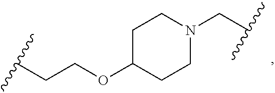 [SMILES: *CCOC1CCN(C*)CC1] | (164) |
| 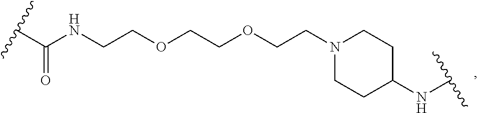 [SMILES: *NC1CCN(CCOCCOCCNC(*)=O)CC1] | (165) |
| 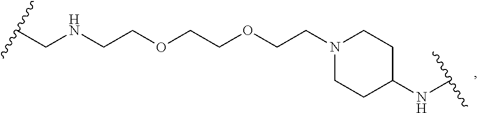 [SMILES: *CNCCOCCOCCN1CCC(N*)CC1] | (166) |
| 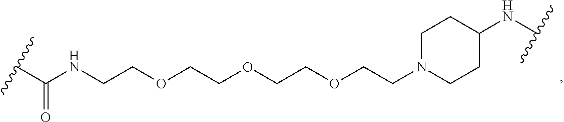 [SMILES: *NC1CCN(CCOCCOCCOCCNC(*)=O)CC1] | (167) |
| 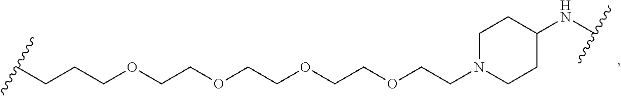 [SMILES: *CCCOCCOCCOCCOCCN1CCC(N*)CC1] | (168) |
| 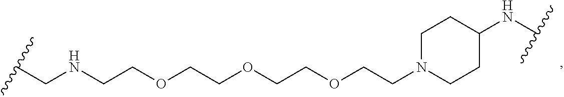 [SMILES: *CNCCOCCOCCOCCN1CCC(N*)CC1] | (169) |
| 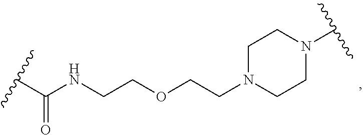 [SMILES: *C(=O)NCCOCCN1CCN(*)CC1] | (170) |
| 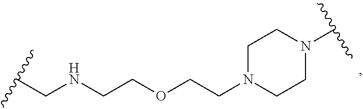 [SMILES: *CNCCOCCN1CCN(*)CC1] | (171) |
| 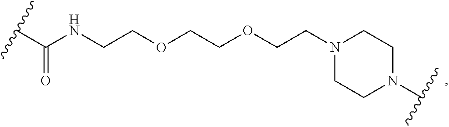 [SMILES: *C(=O)NCCOCCOCCN1CCN(*)CC1] | (172) |
| 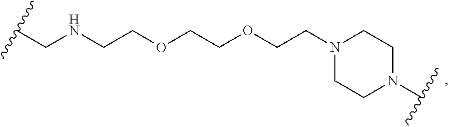 [SMILES: *CNCCOCCOCCN1CCN(*)CC1] | (173) |
| 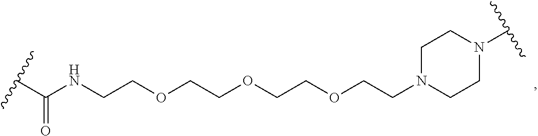 [SMILES: *C(=O)NCCOCCOCCOCCN1CCN(*)CC1] | (174) |
| 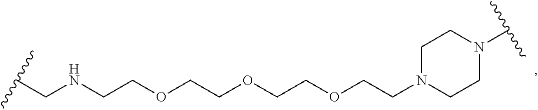 [SMILES: *CNCCOCCOCCOCCN1CCN(*)CC1] | (175) |
| 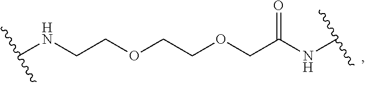 [SMILES: *NCCOCCOCC(=O)N*] | (176) |
| 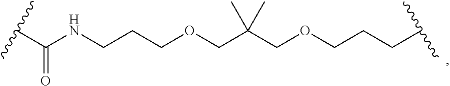 [SMILES: *CCCOCC(C)(C)COCCCNC(*)=O] | (177) |
| 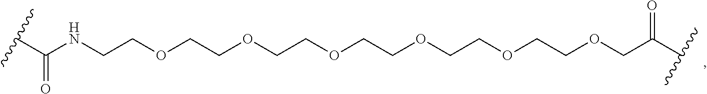 [SMILES: *C(=O)COCCOCCOCCOCCOCCOCCNC(*)=O] | (178) |
| 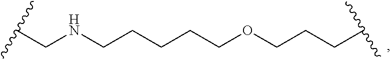 [SMILES: *CCCOCCCCCNC*] | (179) |
| 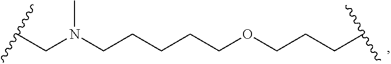 [SMILES: *CCCOCCCCCN(C)C*] | (180) |
| 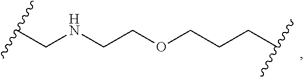 [SMILES: *CCCOCCNC*] | (181) |
| 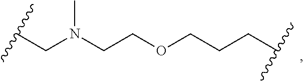 [SMILES: *CCCOCCN(C)C*] | (182) |
| 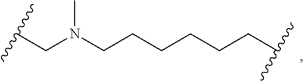 [SMILES: *CCCCCCN(C)C*] | (183) |
| 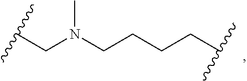 [SMILES: *CCCCN(C)C*] | (184) |
| 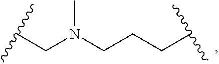 [SMILES: *CCCN(C)C*] | (185) |
| 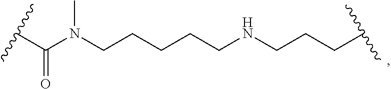 [SMILES: *CCCNCCCCCN(C)C(*)=O] | (186) |
| 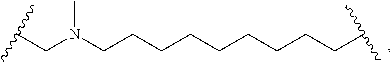 [SMILES: *CCCCCCCCCN(C)C*] | (187) |
| 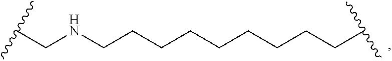 [SMILES: *CCCCCCCCCNC*] | (188) |
| 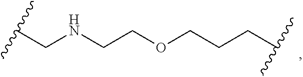 [SMILES: *CCCOCCNC*] | (189) |
| 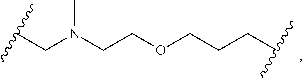 [SMILES: *CCCOCCN(C)C*] | (190) |
| 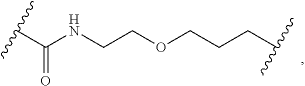 [SMILES: *CCCOCCNC(*)=O] | (191) |
| 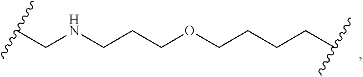 [SMILES: *CCCCOCCCNC*] | (192) |
| 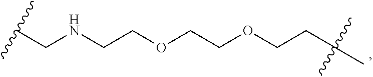 [SMILES: *CCOCCOCCNC*] | (193) |
| 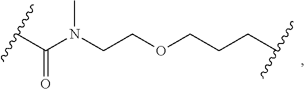 [SMILES: *CCCOCCN(C)C(*)=O] | (194) |
| 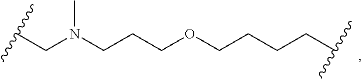 [SMILES: *CCCCOCCCN(C)C*] | (195) |
| 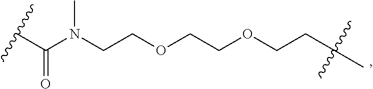 [SMILES: *CCOCCOCCN(C)C(*)=O] | (196) |
| 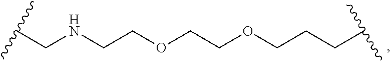 [SMILES: *CCCOCCOCCNC*] | (197) |
| 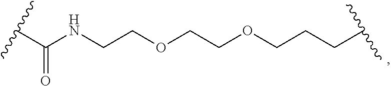 [SMILES: *CCCOCCOCCNC(*)=O] | (198) |
| 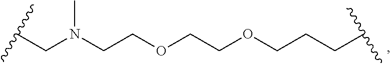 [SMILES: *CCCOCCOCCN(C)C*] | (199) |
| 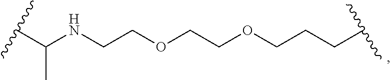 [SMILES: *CCCOCCOCCNC(*)C] | (200) |
| 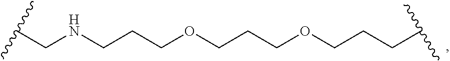 [SMILES: *CCCOCCCOCCCNC*] | (201) |
| 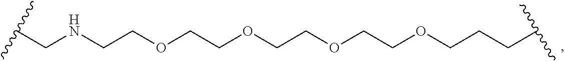 [SMILES: *CCCOCCOCCOCCOCCNC*] | (202) |
| 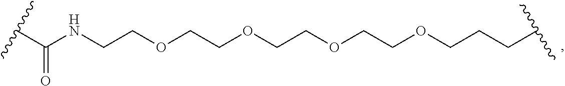 [SMILES: *CCCOCCOCCOCCOCCNC(*)=O] | (203) |
| 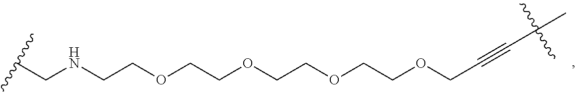 [SMILES: *C#CCOCCOCCOCCOCCNC*] | (204) |
| 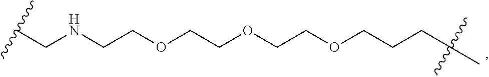 [SMILES: *CCCOCCOCCOCCNC*] | (205) |
| 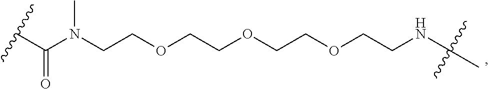 [SMILES: *NCCOCCOCCOCCN(C)C(*)=O] | (206) |
| 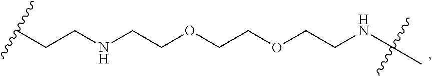 [SMILES: *CCNCCOCCOCCN*] | (207) |
| 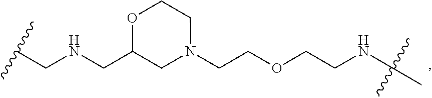 [SMILES: *CNCC1CN(CCOCCN*)CCO1] | (208) |
| 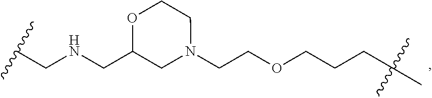 [SMILES: *CCCOCCN1CCOC(CNC*)C1] | (209) |
| 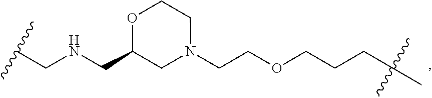 [SMILES: *CCCOCCN1CCO[C@H](CNC*)C1] | (210) |
| 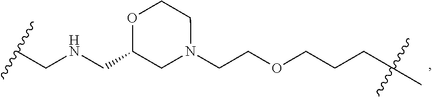 [SMILES: *CCCOCCN1CCO[C@@H](CNC*)C1] | (211) |
| 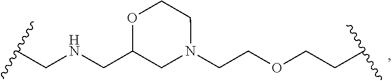 [SMILES: *CCOCCN1CCOC(CNC*)C1] | (212) |
| 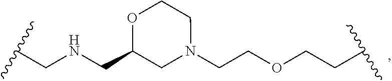 [SMILES: *CCOCCN1CCO[C@H](CNC*)C1] | (213) |
| 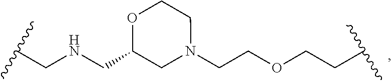 [SMILES: *CCOCCN1CCO[C@@H](CNC*)C1] | (214) |
| 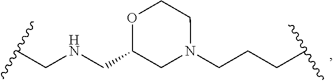 [SMILES: *CCCN1CCO[C@@H](CNC*)C1] | (215) |
| 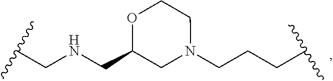 [SMILES: *CCCN1CCO[C@H](CNC*)C1] | (216) |
| 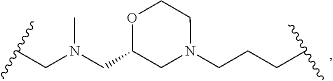 [SMILES: *CCCN1CCO[C@@H](CN(C)C*)C1] | (217) |
| 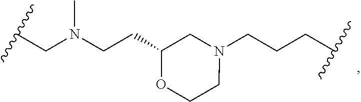 [SMILES: *CCCN1CCO[C@H](CCN(C)C*)C1] | (218) |
| 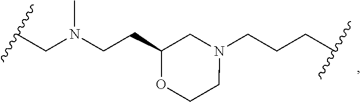 [SMILES: *CCCN1CCO[C@@H](CCN(C)C*)C1] | (219) |
| 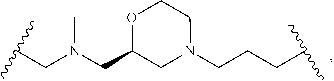 [SMILES: *CCCN1CCO[C@H](CN(C)C*)C1] | (220) |
| 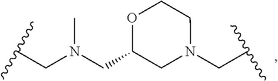 [SMILES: *CN(C)C[C@H]1CN(C*)CCO1] | (221) |
| 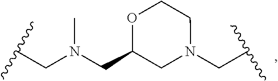 [SMILES: *CN(C)C[C@@H]1CN(C*)CCO1] | (222) |
| 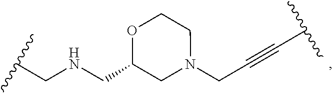 [SMILES: *C#CCN1CCO[C@@H](CNC*)C1] | (223) |
| 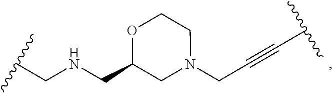 [SMILES: *C#CCN1CCO[C@H](CNC*)C1] | (224) |
| 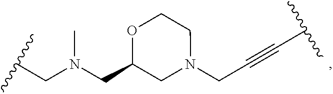 [SMILES: *C#CCN1CCO[C@H](CN(C)C*)C1] | (225) |
| 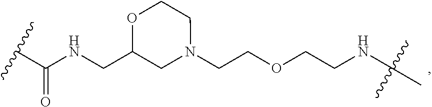 [SMILES: *NCCOCCN1CCOC(CNC(*)=O)C1] | (226) |
| 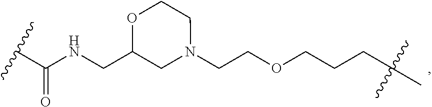 [SMILES: *CCCOCCN1CCOC(CNC(*)=O)C1] | (227) |
| 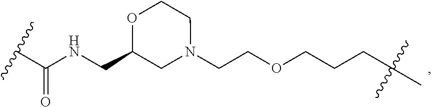 [SMILES: *CCCOCCN1CCO[C@H](CNC(*)=O)C1] | (228) |
| 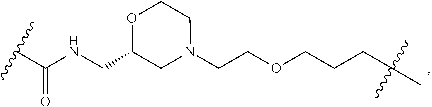 [SMILES: *CCCOCCN1CCO[C@@H](CNC(*)=O)C1] | (229) |
| 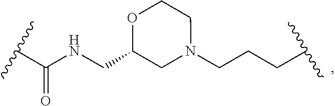 [SMILES: *CCCN1CCO[C@@H](CNC(*)=O)C1] | (230) |
| 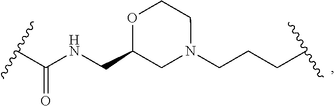 [SMILES: *CCCN1CCO[C@H](CNC(*)=O)C1] | (231) |
| 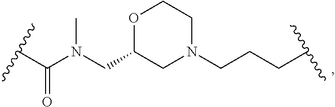 [SMILES: *CCCN1CCO[C@@H](CN(C)C(*)=O)C1] | (232) |
| 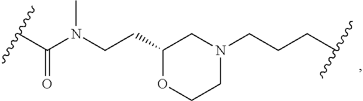 [SMILES: *CCCN1CCO[C@H](CCN(C)C(*)=O)C1] | (233) |
| 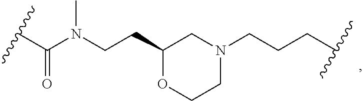 [SMILES: *CCCN1CCO[C@@H](CCN(C)C(*)=O)C1] | (234) |
| 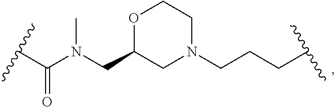 [SMILES: *CCCN1CCO[C@H](CN(C)C(*)=O)C1] | (235) |
| 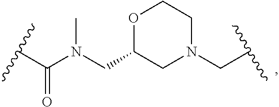 [SMILES: *CN1CCO[C@@H](CN(C)C(*)=O)C1] | (236) |
| 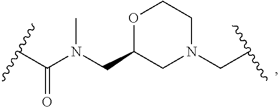 [SMILES: *CN1CCO[C@H](CN(C)C(*)=O)C1] | (237) |
| 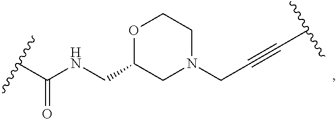 [SMILES: *C#CCN1CCO[C@@H](CNC(*)=O)C1] | (238) |
| 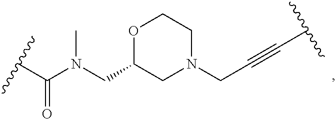 [SMILES: *C#CCN1CCO[C@@H](CN(C)C(*)=O)C1] | (239) |
| 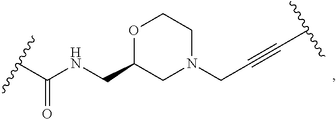 [SMILES: *C#CCN1CCO[C@H](CNC(*)=O)C1] | (240) |
| 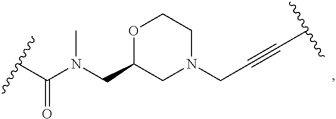 [SMILES: *C#CCN1CCO[C@H](CN(C)C(*)=O)C1] | (241) |
| 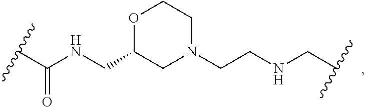 [SMILES: *CNCCN1CCO[C@@H](CNC(*)=O)C1] | (242) |
| 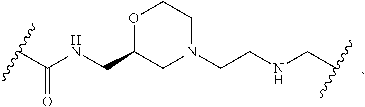 [SMILES: *CNCCN1CCO[C@H](CNC(*)=O)C1] | (243) |
| 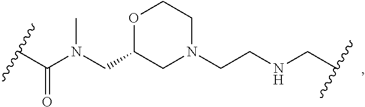 [SMILES: *CNCCN1CCO[C@@H](CN(C)C(*)=O)C1] | (244) |
| 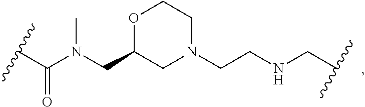 [SMILES: *CNCCN1CCO[C@H](CN(C)C(*)=O)C1] | (245) |
| 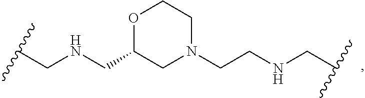 [SMILES: *CNCCN1CCO[C@@H](CNC*)C1] | (246) |
| 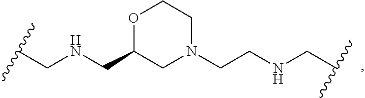 [SMILES: *CNCCN1CCO[C@H](CNC*)C1] | (247) |
| 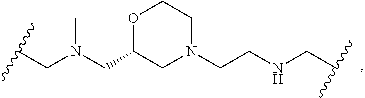 [SMILES: *CNCCN1CCO[C@@H](CN(C)C*)C1] | (248) |
| 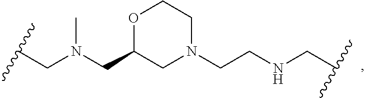 [SMILES: *CNCCN1CCO[C@H](CN(C)C*)C1] | (249) |
| 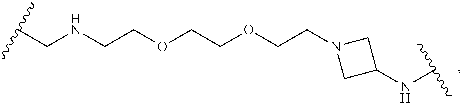 [SMILES: *CNCCOCCOCCN1CC(N*)C1] | (250) |
| 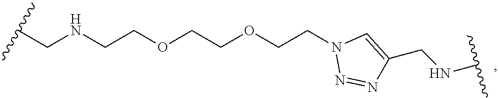 [SMILES: *CNCCOCCOCCn1cc(CN*)nn1] | (251) |
| 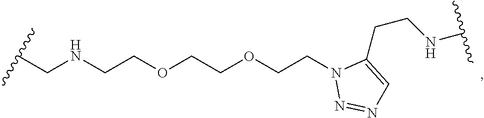 [SMILES: *CNCCOCCOCCn1nncc1CCN*] | (253) |
| 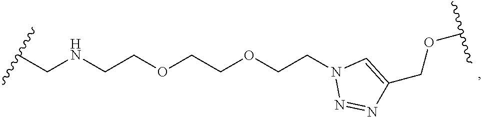 [SMILES: *CNCCOCCOCCn1cc(CO*)nn1] | (254) |
| 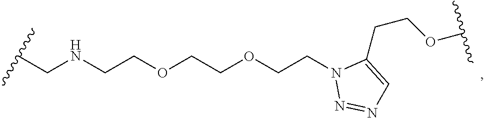 [SMILES: *CNCCOCCOCCn1nncc1CCO*] | (255) |
| 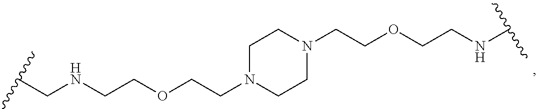 [SMILES: *CNCCOCCN1CCN(CCOCCN*)CC1] | (256) |
| 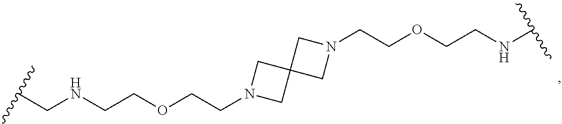 [SMILES: *CNCCOCCN1CC2(CN(CCOCCN*)C2)C1] | (257) |
| 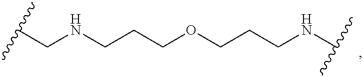 [SMILES: *CNCCCOCCCN*] | (258) |
| 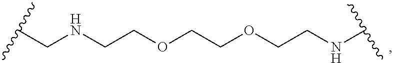 [SMILES: *CNCCOCCOCCN*] | (259) |
| 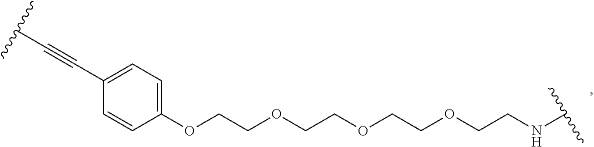 [SMILES: *C#Cc1ccc(OCCOCCOCCOCCN*)cc1] | (260) |
| 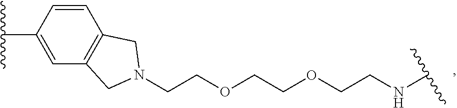 [SMILES: *NCCOCCOCCN1Cc2ccc(*)cc2C1] | (261) |
| 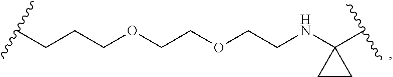 [SMILES: *CCCOCCOCCNC1(*)CC1] | (262) |
| 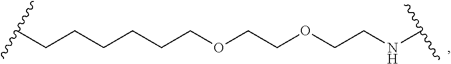 [SMILES: *CCCCCCOCCOCCN*] | (263) |
| 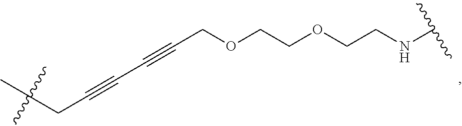 [SMILES: *CC#CC#CCOCCOCCN*] | (264) |
| 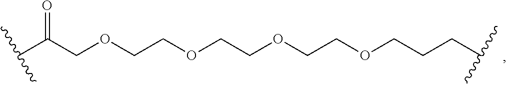 [SMILES: *CCCOCCOCCOCCOCC(*)=O] | (265) |
| 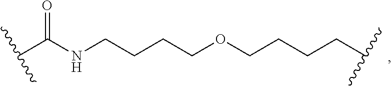 [SMILES: *CCCCOCCCCNC(*)=O] | (266) |
| 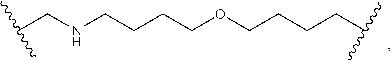 [SMILES: *CCCCOCCCCNC*] | (267) |
| 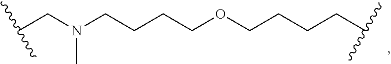 [SMILES: *CCCCOCCCCN(C)C*] | (268) |
| 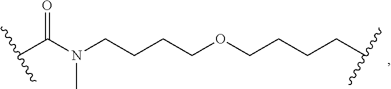 [SMILES: *CCCCOCCCCN(C)C(*)=O] | (269) |
| 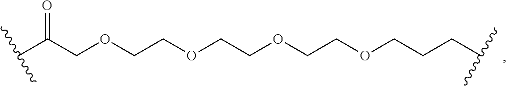 [SMILES: *CCCOCCOCCOCCOCC(*)=O] | (270) |
| 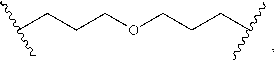 [SMILES: *CCCOCCC*] | (271) |
| 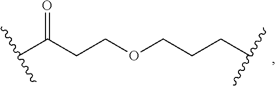 [SMILES: *CCCOCCC(*)=O] | (272) |
| 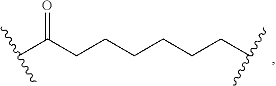 [SMILES: *CCCCCCC(*)=O] | (273) |
| 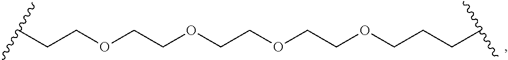 [SMILES: *CCCOCCOCCOCCOCC*] | (274) |
| 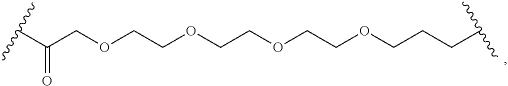 [SMILES: *CCCOCCOCCOCCOCC(*)=O] | (275) |
| 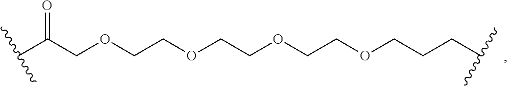 [SMILES: *CCCOCCOCCOCCOCC(*)=O] | (276) |
| 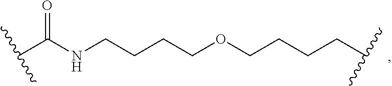 [SMILES: *CCCCOCCCCNC(*)=O] | (277) |
| 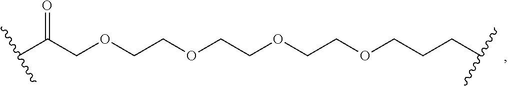 [SMILES: *CCCOCCOCCOCCOCC(*)=O] | (278) |
| 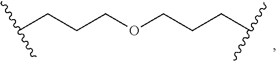 [SMILES: *CCCOCCC*] | (279) |
| 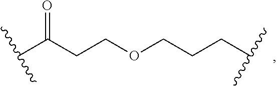 [SMILES: *CCCOCCC(*)=O] | (280) |
| 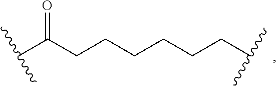 [SMILES: *CCCCCCC(*)=O] | (281) |
| 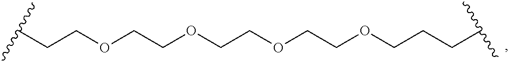 [SMILES: *CCCOCCOCCOCCOCC*] | (282) |
| 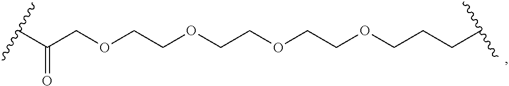 [SMILES: *CCCOCCOCCOCCOCC(*)=O] | (283) |
| 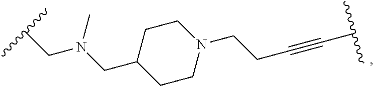 [SMILES: *C#CCCN1CCC(CN(C)C*)CC1] | (284) |
| 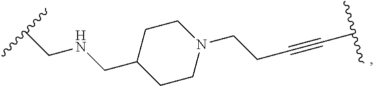 [SMILES: *C#CCCN1CCC(CNC*)CC1] | (285) |
| 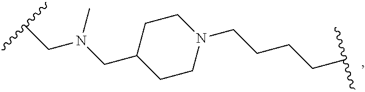 [SMILES: *CCCCN1CCC(CN(C)C*)CC1] | (286) |
| 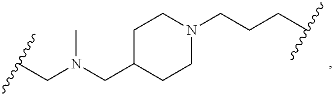 [SMILES: *CCCN1CCC(CN(C)C*)CC1] | (287) |
| 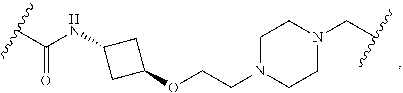 [SMILES: *CN1CCN(CCO[C@H]2C[C@H](NC(*)=O)C2)CC1] | (288) |
| 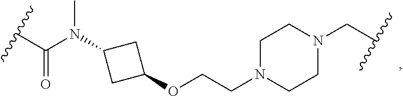 [SMILES: *CN1CCN(CCO[C@H]2C[C@H](N(C)C(*)=O)C2)CC1] | (289) |
| 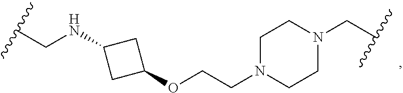 [SMILES: *CN[C@H]1C[C@H](OCCN2CCN(C*)CC2)C1] | (290) |
| 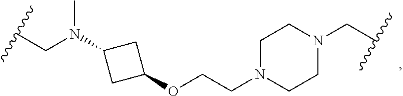 [SMILES: *CN1CCN(CCO[C@H]2C[C@H](N(C)C*)C2)CC1] | (291) |
| 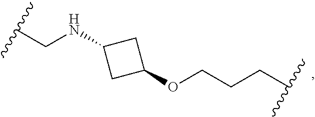 [SMILES: *CCCO[C@H]1C[C@H](NC*)C1] | (292) |
| 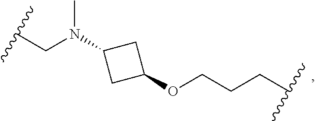 [SMILES: *CCCO[C@H]1C[C@H](N(C)C*)C1] | (293) |
| 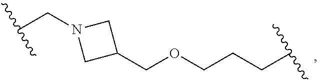 [SMILES: *CCCOCC1CN(C*)C1] | (294) |
| 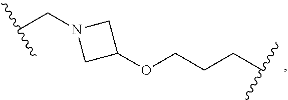 [SMILES: *CCCOC1CN(C*)C1] | (295) |
| 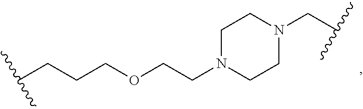 [SMILES: *CCCOCCN1CCN(C*)CC1] | (296) |
| 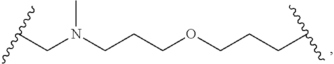 [SMILES: *CCCOCCCN(C)C*] | (297) |
| 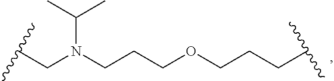 [SMILES: *CCCOCCCN(C*)C(C)C] | (298) |
| 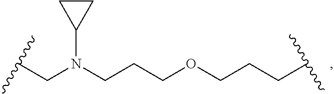 [SMILES: *CCCOCCCN(C*)C1CC1] | (299) |
| 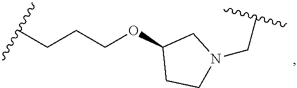 [SMILES: *CCCO[C@@H]1CCN(C*)C1] | (300) |
| 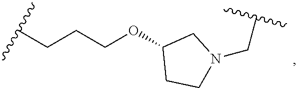 [SMILES: *CCCO[C@H]1CCN(C*)C1] | (301) |
| 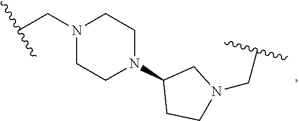 [SMILES: *CN1CCN([C@@H]2CCN(C*)C2)CC1] | (302) |
| 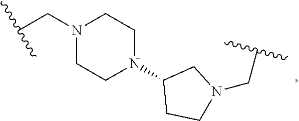 [SMILES: *CN1CCN([C@H]2CCN(C*)C2)CC1] | (303) |
| 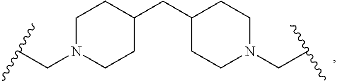 [SMILES: *CN1CCC(CC2CCN(C*)CC2)CC1] | (304) |
| 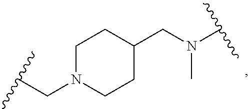 [SMILES: *CN1CCC(CN(*)C)CC1] | (305) |
| 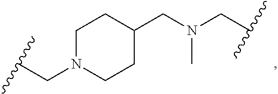 [SMILES: *CN(C)CC1CCN(C*)CC1] | (306) |
| 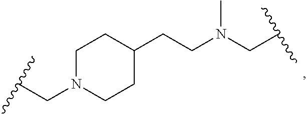 [SMILES: *CN(C)CCC1CCN(C*)CC1] | (307) |
| 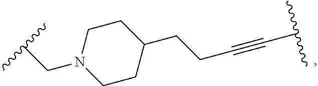 [SMILES: *C#CCCC1CCN(C*)CC1] | (308) |
| 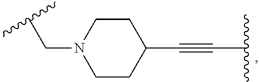 [SMILES: *C#CC1CCN(C*)CC1] | (309) |
| 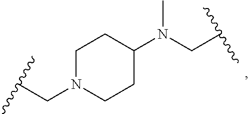 [SMILES: *CN1CCC(N(C)C*)CC1] | (310) |
| 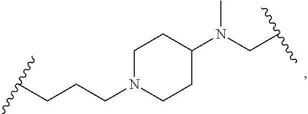 [SMILES: *CCCN1CCC(N(C)C*)CC1] | (311) |
| 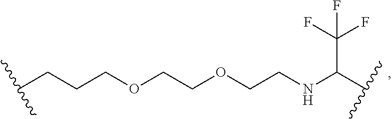 [SMILES: *CCCOCCOCCNC(*)C(F)(F)F] | (312) |
| 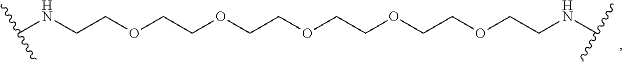 [SMILES: *NCCOCCOCCOCCOCCOCCN*] | (313) |
| 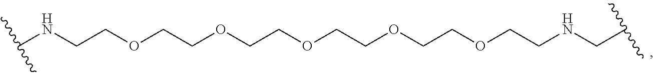 [SMILES: *CNCCOCCOCCOCCOCCOCCN*] | (314) |
| 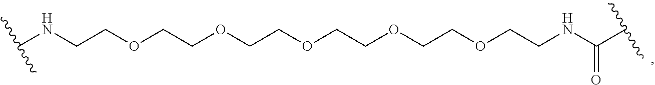 [SMILES: *NCCOCCOCCOCCOCCOCCNC(*)=O] | (315) |
| 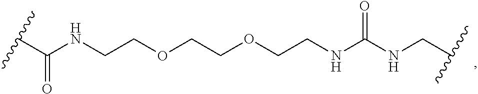 [SMILES: *CNC(=O)NCCOCCOCCNC(*)=O] | (316) |
| 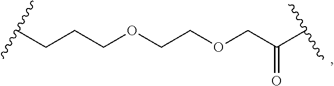 [SMILES: *CCCOCCOCC(*)=O] | (317) |
| 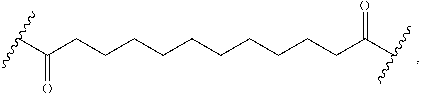 [SMILES: *C(=O)CCCCCCCCCCC(*)=O] | (318) |
| 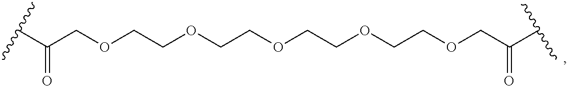 [SMILES: *C(=O)COCCOCCOCCOCCOCC(*)=O] | (319) |
| 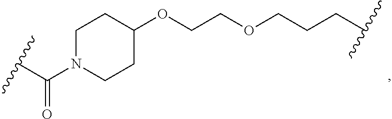 [SMILES: *CCCOCCOC1CCN(C(*)=O)CC1] | (320) |
| 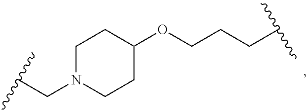 [SMILES: *CCCOC1CCN(C*)CC1] | (321) |
| 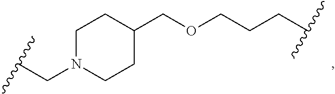 [SMILES: *CCCOCC1CCN(C*)CC1] | (322) |
| 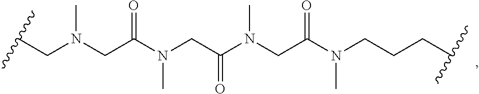 [SMILES: *CCCN(C)C(=O)CN(C)C(=O)CN(C)C(=O)CN(C)C*] | (323) |
| 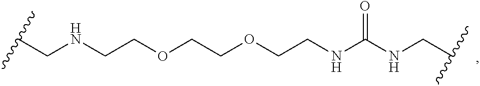 [SMILES: *CNCCOCCOCCNC(=O)NC*] | (324) |
| 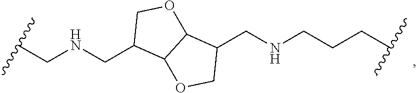 [SMILES: *CCCNCC1COC2C(CNC*)COC12] | (325) |
| 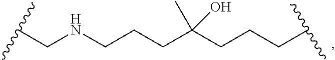 [SMILES: *CCCC(C)(O)CCCNC*] | (326) |
| 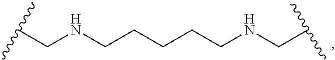 [SMILES: *CNCCCCCNC*] | (327) |
| 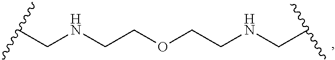 [SMILES: *CNCCOCCNC*] | (328) |
| 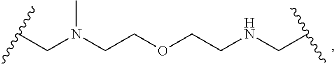 [SMILES: *CNCCOCCN(C)C*] | (329) |
| 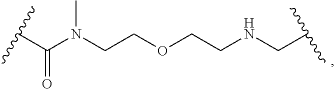 [SMILES: *CNCCOCCN(C)C(*)=O] | (330) |
| 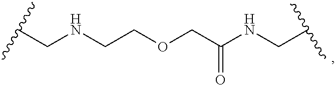 [SMILES: *CNCCOCC(=O)NC*] | (331) |
| 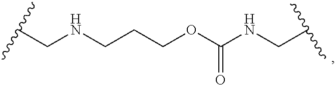 [SMILES: *CNCCCOC(=O)NC*] | (332) |
| 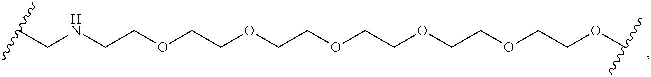 [SMILES: *CNCCOCCOCCOCCOCCOCCO*] | (333) |
| 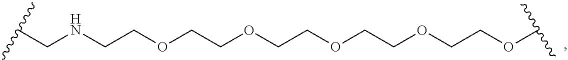 [SMILES: *CNCCOCCOCCOCCOCCO*] | (334) |
| 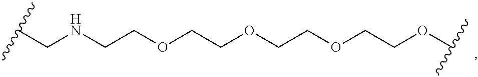 [SMILES: *CNCCOCCOCCOCCO*] | (335) |
| 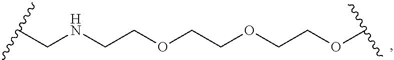 [SMILES: *CNCCOCCOCCO*] | (336) |
| 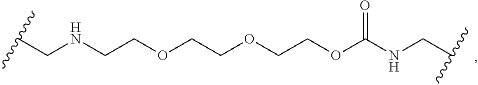 [SMILES: *CNCCOCCOCCOC(=O)NC*] | (337) |
| 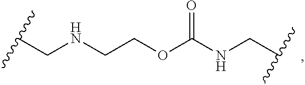 [SMILES: *CNCCOC(=O)NC*] | (338) |
| 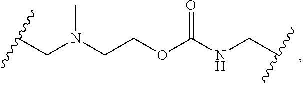 [SMILES: *CNC(=O)OCCN(C)C*] | (339) |
| 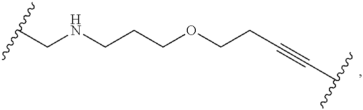 [SMILES: *C#CCCOCCCNC*] | (340) |
| 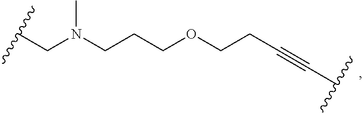 [SMILES: *C#CCCOCCCN(C)C*] | (341) |
| 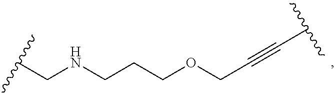 [SMILES: *C#CCOCCCNC*] | (342) |
| 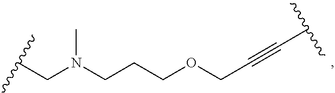 [SMILES: *C#CCOCCCN(C)C*] | (343) |
| 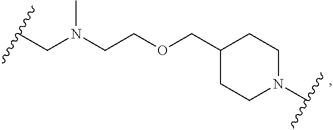 [SMILES: *CN(C)CCOCC1CCN(*)CC1] | (344) |
| 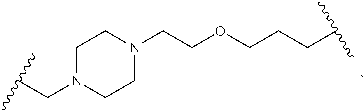 [SMILES: *CCCOCCN1CCN(C*)CC1] | (345) |
| 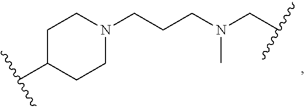 [SMILES: *CN(C)CCCN1CCC(*)CC1] | (346) |
| 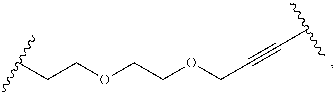 [SMILES: *C#CCOCCOCC*] | (347) |
| 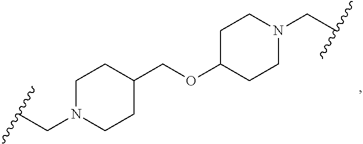 [SMILES: *CN1CCC(COC2CCN(C*)CC2)CC1] | (348) |
| 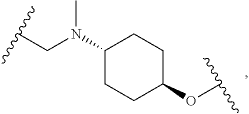 [SMILES: *CN(C)[C@H]1CC[C@H](O*)CC1] | (349) |
| 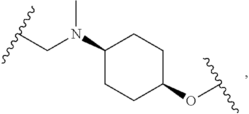 [SMILES: *CN(C)[C@H]1CC[C@@H](O*)CC1] | (350) |
| 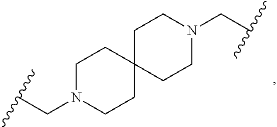 [SMILES: *CN1CCC2(CC1)CCN(C*)CC2] | (351) |
| 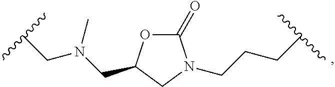 [SMILES: *CCCN1C[C@@H](CN(C)C*)OC1=O] | (352) |
| 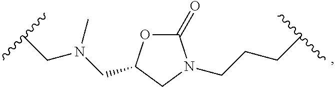 [SMILES: *CCCN1C[C@H](CN(C)C*)OC1=O] | (353) |
| 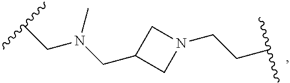 [SMILES: *CCN1CC(CN(C)C*)C1] | (354) |
| 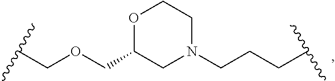 [SMILES: *CCCN1CCO[C@@H](COC*)C1] | (355) |
| 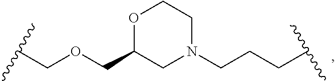 [SMILES: *CCCN1CCO[C@H](COC*)C1] | (356) |
| 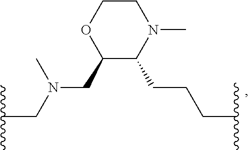 [SMILES: *CCC[C@@H]1[C@@H](CN(C)C*)OCCN1C] | (357) |
| 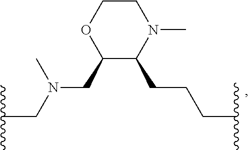 [SMILES: *CCC[C@H]1[C@@H](CN(C)C*)OCCN1C] | (358) |
| 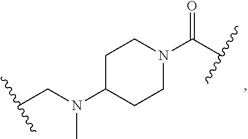 [SMILES: *CN(C)C1CCN(C(*)=O)CC1] | (359) |
| 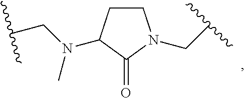 [SMILES: *CN1CCC(N(C)C*)C1=O] | (360) |
| 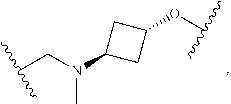 [SMILES: *CN(C)[C@H]1C[C@H](O*)C1] | (361) |
| 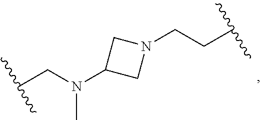 [SMILES: *CCN1CC(N(C)C*)C1] | (362) |
| 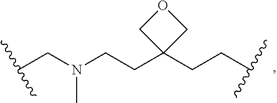 [SMILES: *CCC1(CCN(C)C*)COC1] | (363) |
| 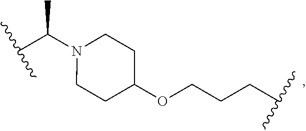 [SMILES: *CCCOC1CCN([C@@H](*)C)CC1] | (364) |
| 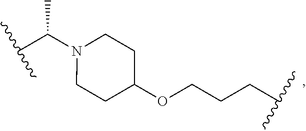 [SMILES: *CCCOC1CCN([C@H](*)C)CC1] | (365) |
| 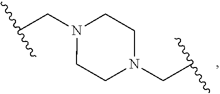 [SMILES: *CN1CCN(C*)CC1] | (366) |
| 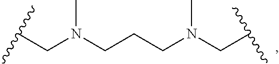 [SMILES: *CN(C)CCCN(C)C*] | (367) |
| 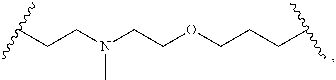 [SMILES: *CCCOCCN(C)CC*] | (368) |
| 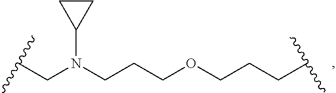 [SMILES: *CCCOCCCN(C*)C1CC1] | (369) |
| 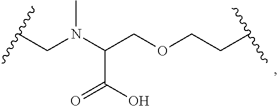 [SMILES: *CCOCC(C(=O)O)N(C)C*] | (370) |
| 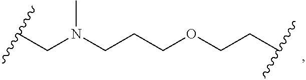 [SMILES: *CCOCCCN(C)C*] | (371) |
| 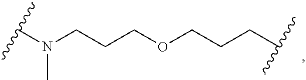 [SMILES: *CCCOCCCN(*)C] | (372) |
| 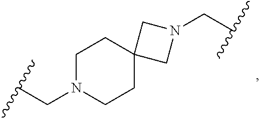 [SMILES: *CN1CCC2(CC1)CN(C*)C2] | (373) |
| 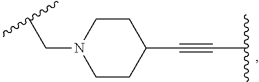 [SMILES: *C#CC1CCN(C*)CC1] | (374) |
| 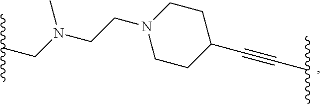 [SMILES: *C#CC1CCN(CCN(C)C*)CC1] | (375) |
| 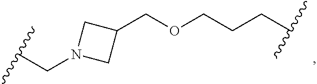 [SMILES: *CCCOCC1CN(C*)C1] | (376) |
| 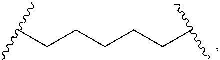 [SMILES: *CCCCC*] | (377) |
| 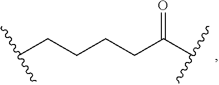 [SMILES: *CCCCC(*)=O] | (378) |
| 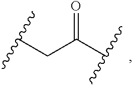 [SMILES: *CC(*)=O] | (379) |
| 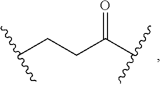 [SMILES: *CCC(*)=O] | (380) |
| 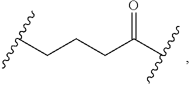 [SMILES: *CCCC(*)=O] | (381) |
| 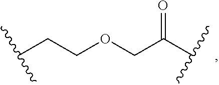 [SMILES: *CCOCC(*)=O] | (382) |
| 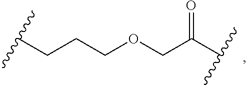 [SMILES: *CCCOCC(*)=O] | (383) |
| 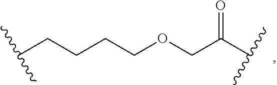 [SMILES: *CCCCOCC(*)=O] | (384) |
| 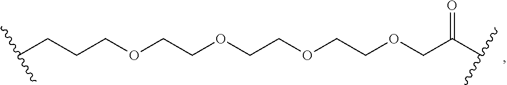 [SMILES: *CCCOCCOCCOCCOCC(*)=O] | (385) |
| 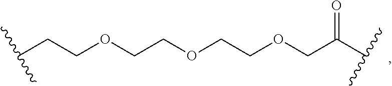 [SMILES: *CCOCCOCCOCC(*)=O] | (386) |
| 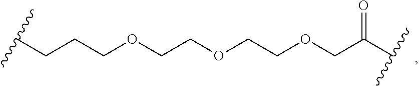 [SMILES: *CCCOCCOCCOCC(*)=O] | (387) |
| 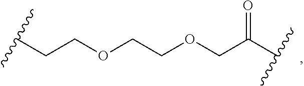 [SMILES: *CCOCCOCC(*)=O] | (388) |
| 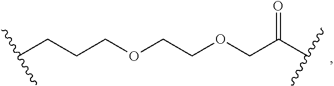 [SMILES: *CCCOCCOCC(*)=O] | (389) |
| 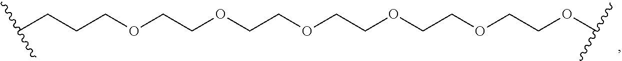 [SMILES: *CCCOCCOCCOCCOCCOCCO*] | (390) |
| 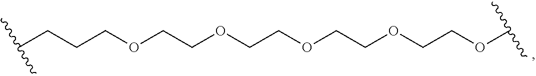 [SMILES: *CCCOCCOCCOCCOCCO*] | (391) |
| 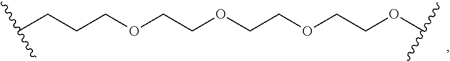 [SMILES: *CCCOCCOCCOCCO*] | (392) |
| 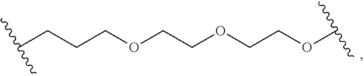 [SMILES: *CCCOCCOCCO*] | (393) |
| 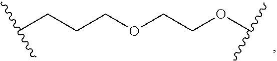 [SMILES: *CCCOCCO*] | (394) |
| 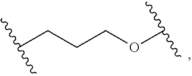 [SMILES: *CCCO*] | (395) |
| 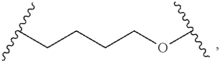 [SMILES: *CCCCO*] | (396) |
| 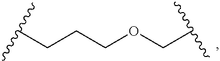 [SMILES: *CCCOC*] | (397) |
| 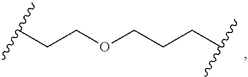 [SMILES: *CCCOCC*] | (398) |
| 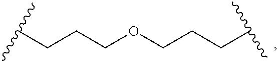 [SMILES: *CCCOCCC*] | (399) |
| 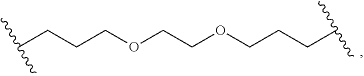 [SMILES: *CCCOCCOCCC*] | (400) |
| 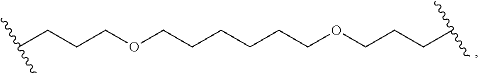 [SMILES: *CCCOCCCCCCOCCC*] | (401) |
| 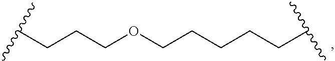 [SMILES: *CCCCCOCCC*] | (402) |
| 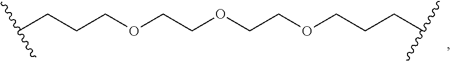 [SMILES: *CCCOCCOCCOCCC*] | (403) |
| 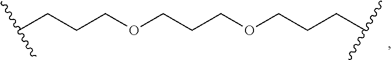 [SMILES: *CCCOCCCOCCC*] | (404) |
| 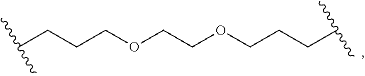 [SMILES: *CCCOCCOCCC*] | (405) |
| 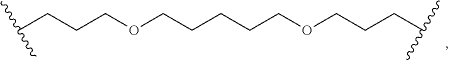 [SMILES: *CCCOCCCCCOCCC*] | (406) |
| 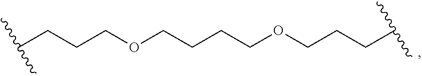 [SMILES: *CCCOCCCCOCCC*] | (407) |
| 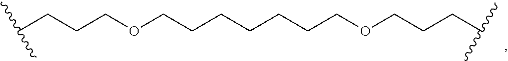 [SMILES: *CCCOCCCCCCCOCCC*] | (408) |
| 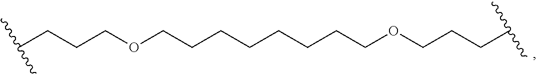 [SMILES: *CCCOCCCCCCCCOCCC*] | (409) |
| 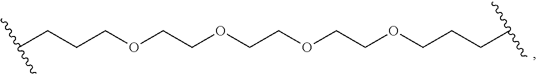 [SMILES: *CCCOCCOCCOCCOCCC*] | (410) |
| 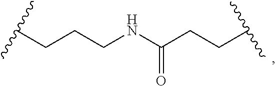 [SMILES: *CCCNC(=O)CC*] | (411) |
| 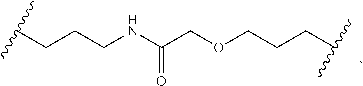 [SMILES: *CCCNC(=O)COCCC*] | (412) |
| 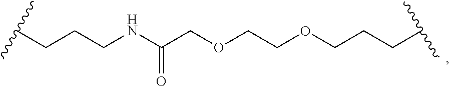 [SMILES: *CCCNC(=O)COCCOCCC*] | (413) |
| 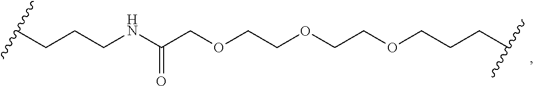 [SMILES: *CCCNC(=O)COCCOCCOCCC*] | (414) |
| 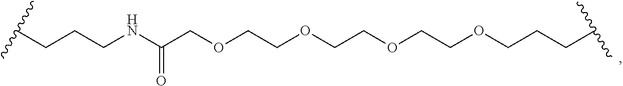 [SMILES: *CCCNC(=O)COCCOCCOCCOCCC*] | (415) |
| 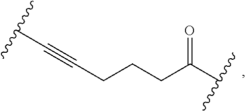 [SMILES: *C#CCCCC(*)=O] | (416) |
| 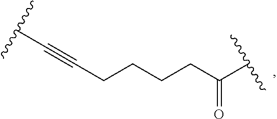 [SMILES: *C#CCCCCC(*)=O] | (417) |
| 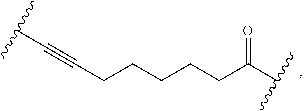 [SMILES: *C#CCCCCCC(*)=O] | (418) |
| 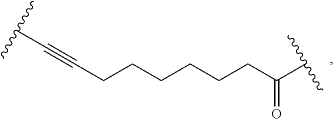 [SMILES: *C#CCCCCCCC(*)=O] | (419) |
| 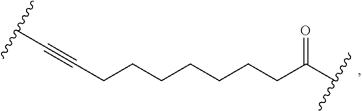 [SMILES: *C#CCCCCCCCC(*)=O] | (420) |
| 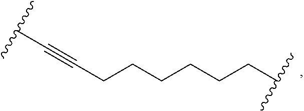 [SMILES: *C#CCCCCCC*] | (421) |
| 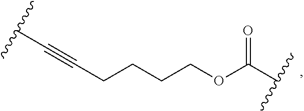 [SMILES: *C#CCCCCOC(*)=O] | (422) |
| 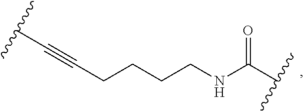 [SMILES: *C#CCCCCNC(*)=O] | (423) |
| 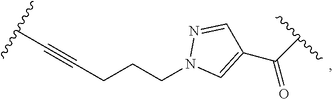 [SMILES: *C#CCCCn1cc(C(*)=O)cn1] | (424) |
| 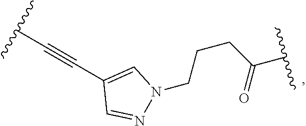 [SMILES: *C#Cc1cnn(CCCC(*)=O)c1] | (425) |
| 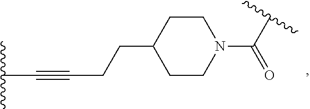 [SMILES: *C#CCCC1CCN(C(*)=O)CC1] | (426) |
| 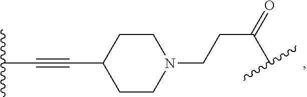 [SMILES: *C#CC1CCN(CCC(*)=O)CC1] | (427) |
| 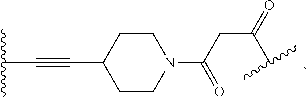 [SMILES: *C#CC1CCN(C(=O)CC(*)=O)CC1] | (428) |
| 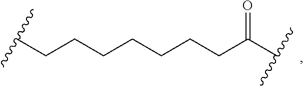 [SMILES: *CCCCCCCC(*)=O] | (429) |
| 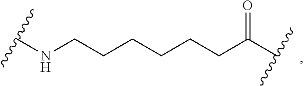 [SMILES: *NCCCCCCC(*)=O] | (430) |
| 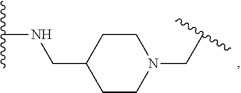 [SMILES: *CN1CCC(CN*)CC1] | (431) |
| 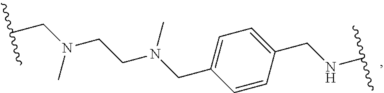 [SMILES: *CN(C)CCN(C)Cc1ccc(CN*)cc1] | (432) |
| 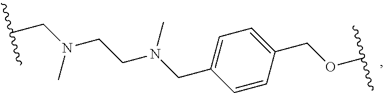 [SMILES: *CN(C)CCN(C)Cc1ccc(CO*)cc1] | (433) |
| 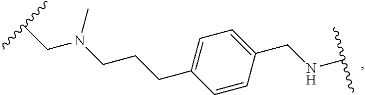 [SMILES: *CN(C)CCCc1ccc(CN*)cc1] | (434) |
| 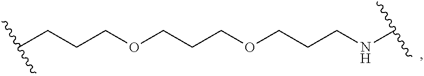 [SMILES: *CCCOCCCOCCCN*] | (435) |
| 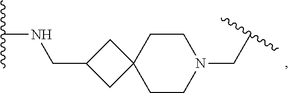 [SMILES: *CN1CCC2(CC1)CC(CN*)C2] | (436) |
| 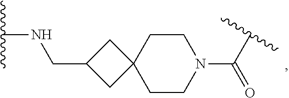 [SMILES: *NCC1CC2(CCN(C(*)=O)CC2)C1] | (437) |
| 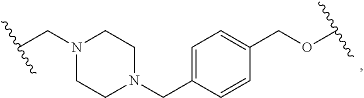 [SMILES: *CN1CCN(Cc2ccc(CO*)cc2)CC1] | (438) |
| 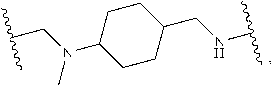 [SMILES: *CN(C)C1CCC(CN*)CC1] | (438) |
| 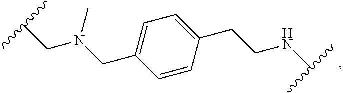 [SMILES: *CN(C)Cc1ccc(CCN*)cc1] | (439) |
| 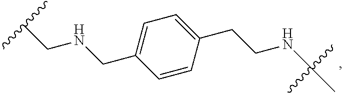 [SMILES: *CNCc1ccc(CCN*)cc1] | (440) |
| 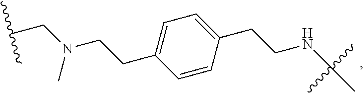 [SMILES: *CN(C)CCc1ccc(CCN*)cc1] | (441) |
| 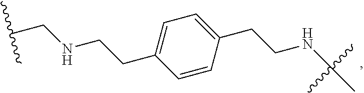 [SMILES: *CNCCc1ccc(CCN*)cc1] | (442) |
| 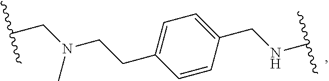 [SMILES: *CN(C)CCc1ccc(CN*)cc1] | (443) |
| 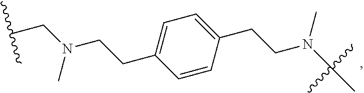 [SMILES: *CN(C)CCc1ccc(CCN(*)C)cc1] | (444) |
| 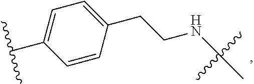 [SMILES: *NCCc1ccc(*)cc1] | (445) |
| 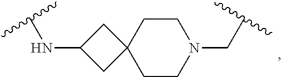 [SMILES: *CN1CCC2(CC1)CC(N*)C2] | (446) |
| 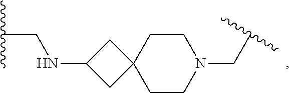 [SMILES: *CNC1CC2(CCN(C*)CC2)C1] | (447) |
| 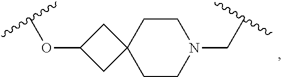 [SMILES: *CN1CCC2(CC1)CC(O*)C2] | (448) |
| 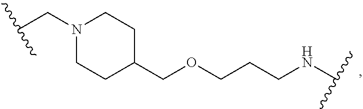 [SMILES: *CN1CCC(COCCCN*)CC1] | (449) |
| 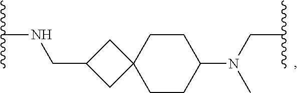 [SMILES: *CN(C)C1CCC2(CC1)CC(CN*)C2] | (450) |
| 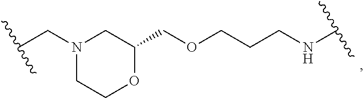 [SMILES: *CN1CCO[C@@H](COCCCN*)C1] | (451) |
| 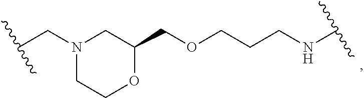 [SMILES: *CN1CCO[C@H](COCCCN*)C1] | (452) |
| 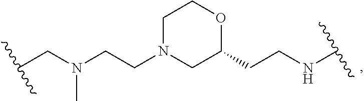 [SMILES: *CN(C)CCN1CCO[C@H](CCN*)C1] | (453) |
| 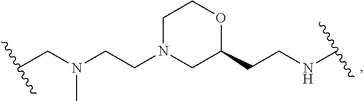 [SMILES: *CN(C)CCN1CCO[C@@H](CCN*)C1] | (454) |
| 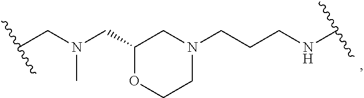 [SMILES: *CN(C)C[C@@H]1CN(CCCN*)CCO1] | (455) |
| 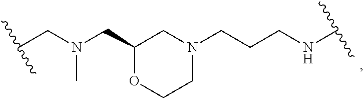 [SMILES: *CN(C)C[C@H]1CN(CCCN*)CCO1] | (456) |
| 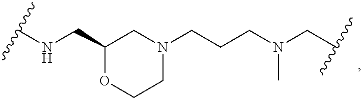 [SMILES: *CN(C)CCCN1CCO[C@@H](CN*)C1] | (457) |
| 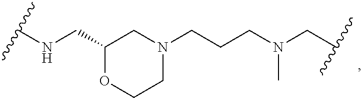 [SMILES: *CN(C)CCCN1CCO[C@H](CN*)C1] | (458) |
| 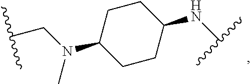 [SMILES: *CN(C)[C@H]1CC[C@@H](N*)CC1] | (459) |
| 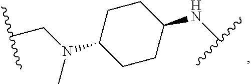 [SMILES: *CN(C)[C@H]1CC[C@H](N*)CC1] | (460) |
| 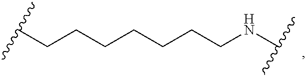 [SMILES: *CCCCCCCN*] | (461) |
| 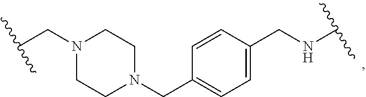 [SMILES: *CN1CCN(Cc2ccc(CN*)cc2)CC1] | (462) |
| 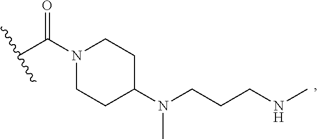 [SMILES: *C(=O)N1CCC(N(C)CCCNC)CC1] | (463) |
| 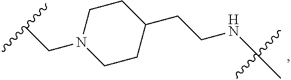 [SMILES: *CN1CCC(CCN*)CC1] | (464) |
| 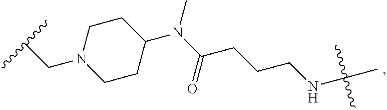 [SMILES: *CN1CCC(N(C)C(=O)CCCN*)CC1] | (465) |
| 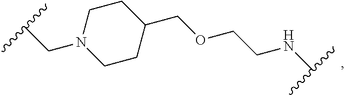 [SMILES: *CN1CCC(COCCN*)CC1] | (466) |
| 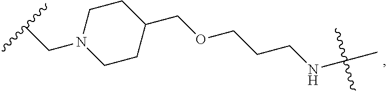 [SMILES: *CN1CCC(COCCCN*)CC1] | (467) |
| 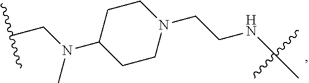 [SMILES: *CN(C)C1CCN(CCN*)CC1] | (468) |
| 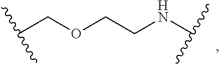 [SMILES: *COCCN*] | (469) |
| 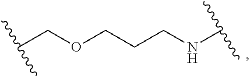 [SMILES: *COCCCN*] | (470) |
| 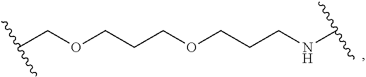 [SMILES: *COCCCOCCCN*] | (471) |
| 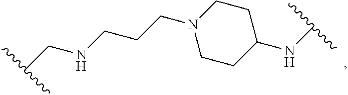 [SMILES: *CNCCCN1CCC(N*)CC1] | (472) |
| 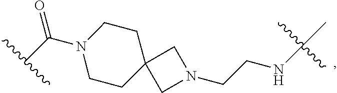 [SMILES: *NCCN1CC2(CCN(C(*)=O)CC2)C1] | (473) |
| 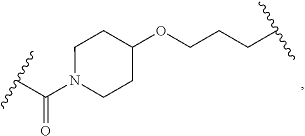 [SMILES: *CCCOC1CCN(C(*)=O)CC1] | (474) |
| 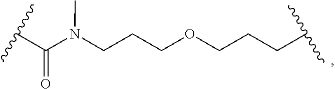 [SMILES: *CCCOCCCN(C)C(*)=O] | (475) |
| 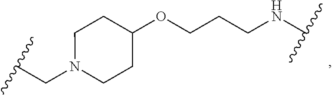 [SMILES: *CN1CCC(OCCCN*)CC1] | (475) |
| 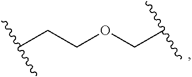 [SMILES: *CCOC*] | (476) |
| 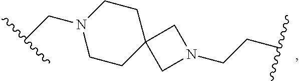 [SMILES: *CCN1CC2(CCN(C*)CC2)C1] | (477) |
| 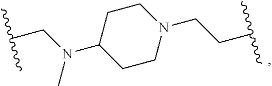 [SMILES: *CCN1CCC(N(C)C*)CC1] | (478) |
| 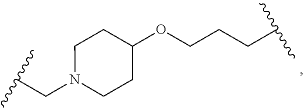 [SMILES: *CCCOC1CCN(C*)CC1] | (479) |
| 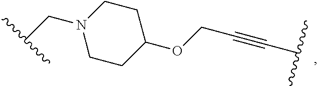 [SMILES: *C#CCOC1CCN(C*)CC1] | (480) |
| 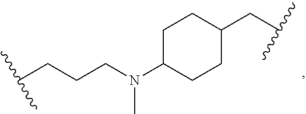 [SMILES: *CCCN(C)C1CCC(C*)CC1] | (481) |
| 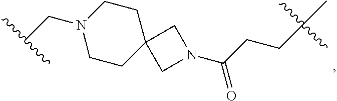 [SMILES: *CCC(=O)N1CC2(CCN(C*)CC2)C1] | (482) |
| 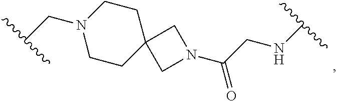 [SMILES: *CN1CCC2(CC1)CN(C(=O)CN*)C2] | (483) |
| 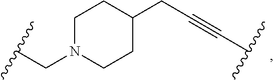 [SMILES: *C#CCC1CCN(C*)CC1] | (484) |
| 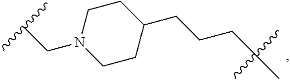 [SMILES: *CCCC1CCN(C*)CC1] | (485) |
| 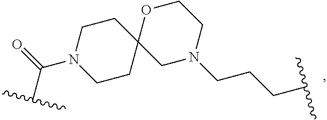 [SMILES: *CCCN1CCOC2(CCN(C(*)=O)CC2)C1] | (486) |
| 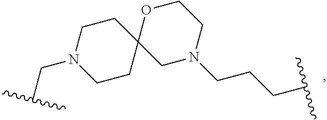 [SMILES: *CCCN1CCOC2(CCN(C*)CC2)C1] | (487) |
| 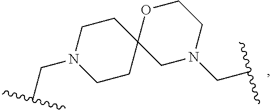 [SMILES: *CN1CCC2(CC1)CN(C*)CCO2] | (488) |
| 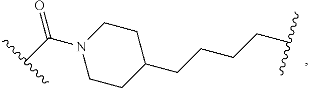 [SMILES: *CCCCC1CCN(C(*)=O)CC1] | (489) |
| 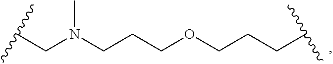 [SMILES: *CCCOCCCN(C)C*] | (490) |
| 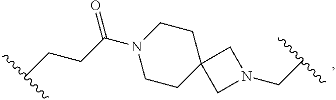 [SMILES: *CCC(=O)N1CCC2(CC1)CN(C*)C2] | (491) |
| 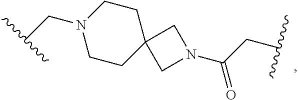 [SMILES: *CC(=O)N1CC2(CCN(C*)CC2)C1] | (492) |
| 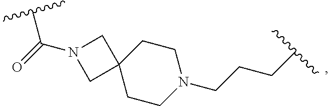 [SMILES: *CCCN1CCC2(CC1)CN(C(*)=O)C2] | (493) |
| 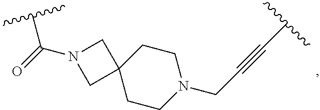 [SMILES: *C#CCN1CCC2(CC1)CN(C(*)=O)C2] | (494) |
| 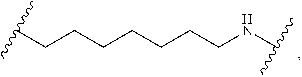 [SMILES: *CCCCCCCN*] | (495) |
| 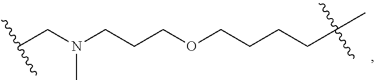 [SMILES: *CCCCOCCCN(C)C*] | (496) |
| 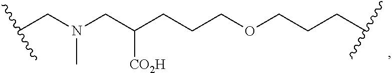 [SMILES: *CCCOCCCC(CN(C)C*)C(=O)O] | (497) |
| 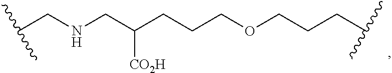 [SMILES: *CCCOCCCC(CNC*)C(=O)O] | (498) |
| 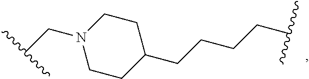 [SMILES: *CCCCC1CCN(C*)CC1] | (499) |
| 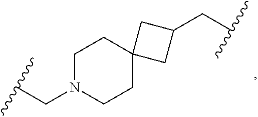 [SMILES: *CC1CC2(CCN(C*)CC2)C1] | (500) |
| 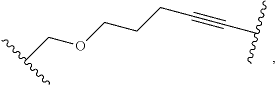 [SMILES: *C#CCCCOC*] | (501) |
| 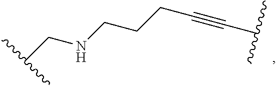 [SMILES: *C#CCCCNC*] | (502) |
| 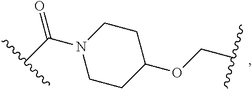 [SMILES: *COC1CCN(C(*)=O)CC1] | (503) |
| 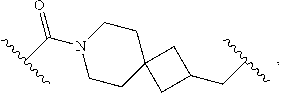 [SMILES: *CC1CC2(CCN(C(*)=O)CC2)C1] | (504) |
| 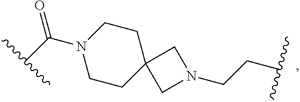 [SMILES: *CCN1CC2(CCN(C(*)=O)CC2)C1] | (505) |
| 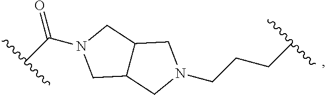 [SMILES: *CCCN1CC2CN(C(*)=O)CC2C1] | (506) |
| 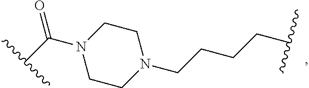 [SMILES: *CCCCN1CCN(C(*)=O)CC1] | (507) |
| 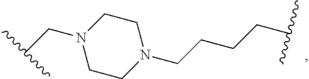 [SMILES: *CCCCN1CCN(C*)CC1] | (508) |
| 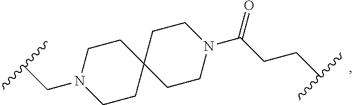 [SMILES: *CCC(=O)N1CCC2(CCN(C*)CC2)CC1] | (509) |
| 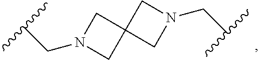 [SMILES: *CN1CC2(C1)CN(C*)C2] | (510) |
| 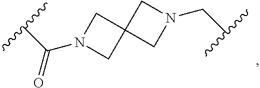 [SMILES: *CN1CC2(C1)CN(C(*)=O)C2] | (511) |
| 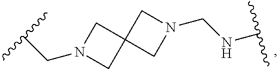 [SMILES: *CN1CC2(C1)CN(CN*)C2] | (512) |
| 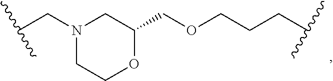 [SMILES: *CCCOC[C@H]1CN(C*)CCO1] | (513) |
| 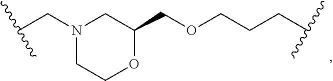 [SMILES: *CCCOC[C@@H]1CN(C*)CCO1] | (514) |
| 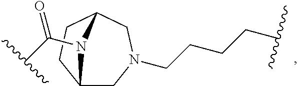 [SMILES: *CCCCN1C[C@H]2CC[C@@H](C1)N2C(*)=O] | (515) |
| 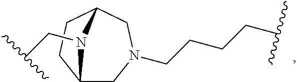 [SMILES: *CCCCN1C[C@H]2CC[C@@H](C1)N2C*] | (516) |
| 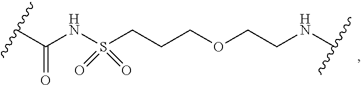 [SMILES: *NCCOCCCS(=O)(=O)NC(*)=O] | (517) |
| 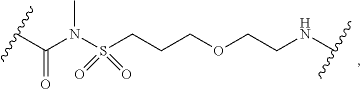 [SMILES: *NCCOCCCS(=O)(=O)N(C)C(*)=O] | (518) |
| 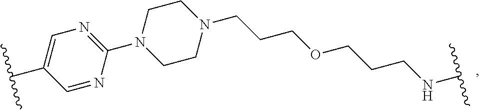 [SMILES: *NCCCOCCCN1CCN(c2ncc(*)cn2)CC1] | (519) |
| 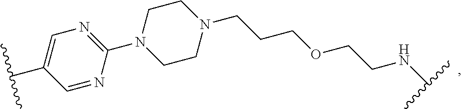 [SMILES: *NCCOCCCN1CCN(c2ncc(*)cn2)CC1] | (520) |
| 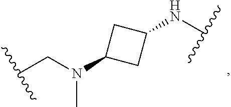 [SMILES: *CN(C)[C@H]1C[C@H](N*)C1] | (521) |
| 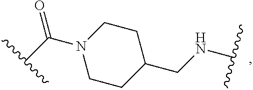 [SMILES: *NCC1CCN(C(*)=O)CC1] | (522) |
| 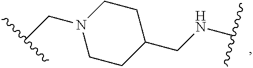 [SMILES: *CN1CCC(CN*)CC1] | (523) |
| 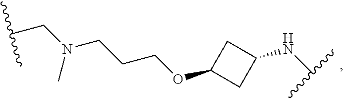 [SMILES: *CN(C)CCCO[C@H]1C[C@H](N*)C1] | (524) |
| 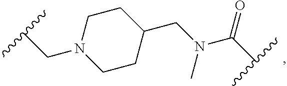 [SMILES: *CN1CCC(CN(C)C(*)=O)CC1] | (525) |
| 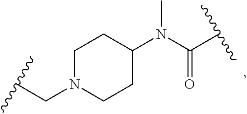 [SMILES: *CN1CCC(N(C)C(*)=O)CC1] | (526) |
| 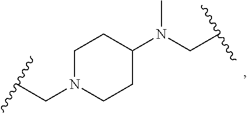 [SMILES: *CN1CCC(N(C)C*)CC1] | (527) |
| 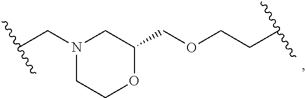 [SMILES: *CCOC[C@H]1CN(C*)CCO1] | (528) |
| 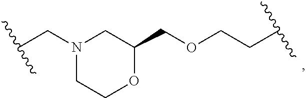 [SMILES: *CCOC[C@@H]1CN(C*)CCO1] | (529) |
| 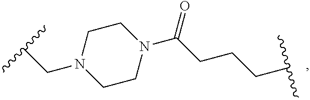 [SMILES: *CCCC(=O)N1CCN(C*)CC1] | (530) |
| 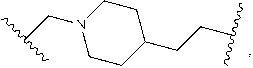 [SMILES: *CCC1CCN(C*)CC1] | (531) |
| 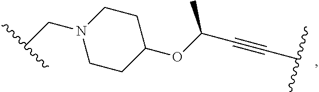 [SMILES: *C#C[C@H](C)OC1CCN(C*)CC1] | (532) |
| 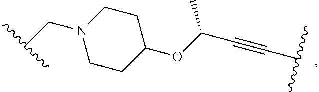 [SMILES: *C#C[C@@H](C)OC1CCN(C*)CC1] | (533) |
| 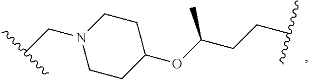 [SMILES: *CC[C@H](C)OC1CCN(C*)CC1] | (534) |
| 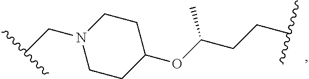 [SMILES: *CC[C@@H](C)OC1CCN(C*)CC1] | (535) |
| 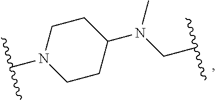 [SMILES: *CN(C)C1CCN(*)CC1] | (536) |
| 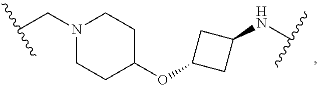 [SMILES: *CN1CCC(O[C@H]2C[C@H](N*)C2)CC1] | (537) |
| 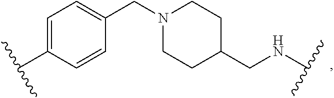 [SMILES: *NCC1CCN(Cc2ccc(*)cc2)CC1] | (538) |
| 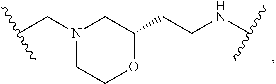 [SMILES: *CN1CCO[C@@H](CCN*)C1] | (539) |
| 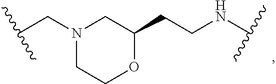 [SMILES: *CN1CCO[C@H](CCN*)C1] | (540) |
| 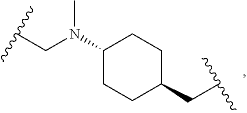 [SMILES: *CN(C)[C@H]1CC[C@H](C*)CC1] | (541) |
| 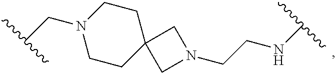 [SMILES: *CN1CCC2(CC1)CN(CCN*)C2] | (542) |
| 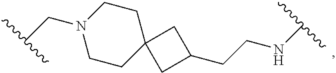 [SMILES: *CN1CCC2(CC1)CC(CCN*)C2] | (543) |
| 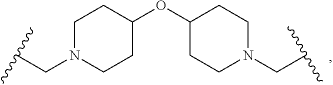 [SMILES: *CN1CCC(OC2CCN(C*)CC2)CC1] | (544) |
| 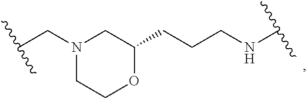 [SMILES: *CN1CCO[C@@H](CCCN*)C1] | (545) |
| 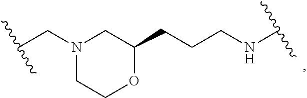 [SMILES: *CN1CCO[C@H](CCCN*)C1] | (546) |
| 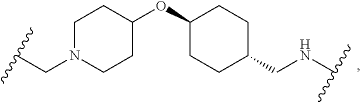 [SMILES: *CN1CCC(O[C@H]2CC[C@H](CN*)CC2)CC1] | (547) |
| 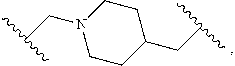 [SMILES: *CC1CCN(C*)CC1] | (548) |
| 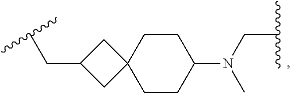 [SMILES: *CC1CC2(CCC(N(C)C*)CC2)C1] | (549) |
| 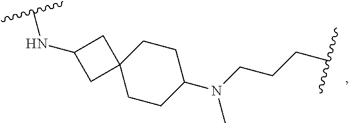 [SMILES: *CCCN(C)C1CCC2(CC1)CC(N*)C2] | (550) |
| 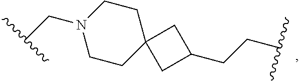 [SMILES: *CCC1CC2(CCN(C*)CC2)C1] | (551) |
| 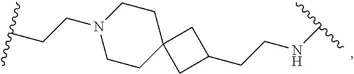 [SMILES: *CCN1CCC2(CC1)CC(CCN*)C2] | (552) |
| 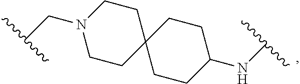 [SMILES: *CN1CCC2(CCC(N*)CC2)CC1] | (553) |
| 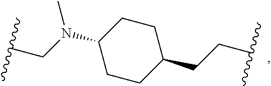 [SMILES: *CC[C@H]1CC[C@H](N(C)C*)CC1] | (554) |
| 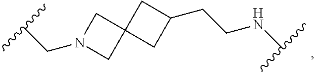 [SMILES: *CN1CC2(CC(CCN*)C2)C1] | (555) |
| 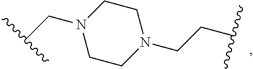 [SMILES: *CCN1CCN(C*)CC1] | (556) |
| 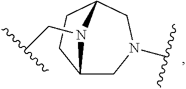 [SMILES: *CN1[C@@H]2CC[C@H]1CN(*)C2] | (557) |
| 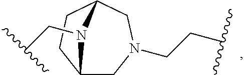 [SMILES: *CCN1C[C@H]2CC[C@@H](C1)N2C*] | (558) |
| 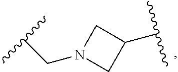 [SMILES: *CN1CC(*)C1] | (559) |
| 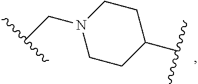 [SMILES: *CN1CCC(*)CC1] | (560) |
| 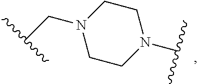 [SMILES: *CN1CCN(*)CC1] | (561) |
| 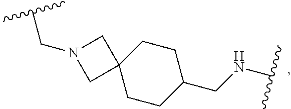 [SMILES: *CN1CC2(CCC(CN*)CC2)C1] | (562) |
| 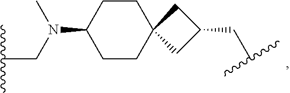 | (563) |
| 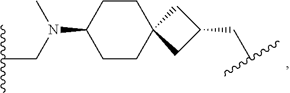 | (564) |
| 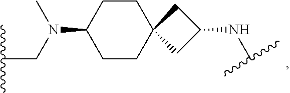 | (565) |
| 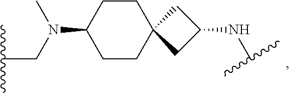 | (566) |
| 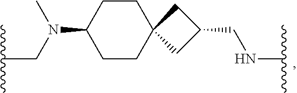 | (567) |
| 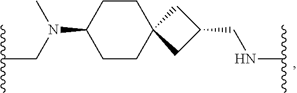 | (568) |
| 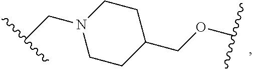 [SMILES: *CN1CCC(CO*)CC1] | (569) |
| 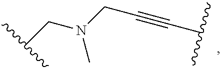 [SMILES: *C#CCN(C)C*] | (570) |
| 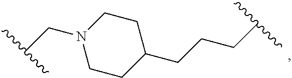 [SMILES: *CCCC1CCN(C*)CC1] | (571) |
| 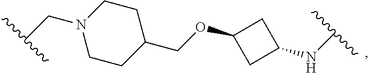 [SMILES: *CN1CCC(CO[C@H]2C[C@H](N*)C2)CC1] | (572) |
| 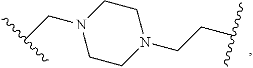 [SMILES: *CCN1CCN(C*)CC1] | (573) |
| 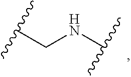 [SMILES: *CN*] | (574) |
| 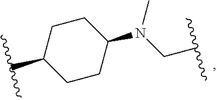 [SMILES: *CN(C)[C@H]1CC[C@@H](*)CC1] | (575) |
| 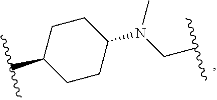 [SMILES: *CN(C)[C@H]1CC[C@H](*)CC1] | (576) |
| 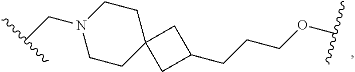 [SMILES: *CN1CCC2(CC1)CC(CCCO*)C2] | (577) |
| 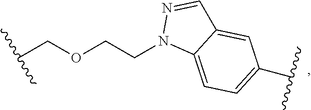 [SMILES: *COCCn1ncc2cc(*)ccc21] | (578) |
| 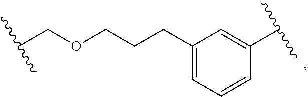 [SMILES: *COCCCc1cccc(*)c1] | (579) |
| 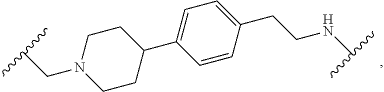 [SMILES: *CN1CCC(c2ccc(CCN*)cc2)CC1] | (580) |
| 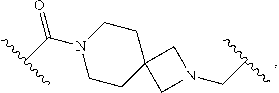 [SMILES: *CN1CC2(CCN(C(*)=O)CC2)C1] | (581) |
| 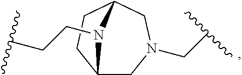 [SMILES: *CCN1[C@@H]2CC[C@H]1CN(C*)C2] | (582) |
| 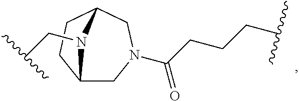 [SMILES: *CCCC(=O)N1C[C@H]2CC[C@@H](C1)N2C*] | (583) |
| 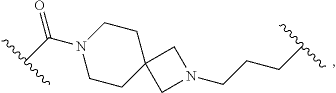 [SMILES: *CCCN1CC2(CCN(C(*)=O)CC2)C1] | (584) |
| 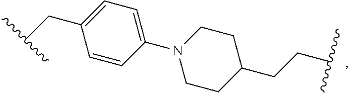 [SMILES: *CCC1CCN(c2ccc(C*)cc2)CC1] | (585) |
| 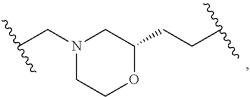 [SMILES: *CC[C@H]1CN(C*)CCO1] | (586) |
| 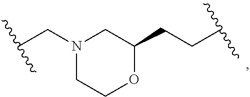 [SMILES: *CC[C@@H]1CN(C*)CCO1] | (587) |
| 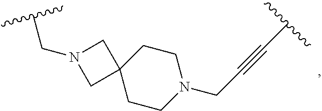 [SMILES: *C#CCN1CCC2(CC1)CN(C*)C2] | (588) |
| 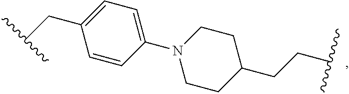 [SMILES: *CCC1CCN(c2ccc(C*)cc2)CC1] | (589) |
| 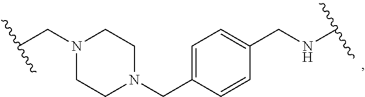 [SMILES: *CN1CCN(Cc2ccc(CN*)cc2)CC1] | (590) |
| 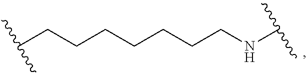 [SMILES: *CCCCCCCN*] | (591) |
| 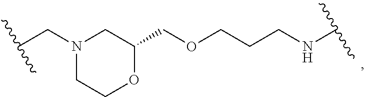 [SMILES: *CN1CCO[C@@H](COCCCN*)C1] | (592) |
| 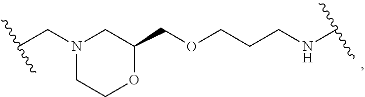 [SMILES: *CN1CCO[C@H](COCCCN*)C1] | (593) |
| 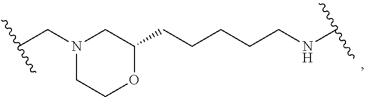 [SMILES: *CN1CCO[C@@H](CCCCCN*)C1] | (594) |
| 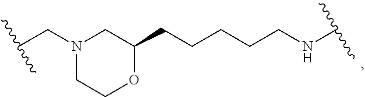 [SMILES: *CN1CCO[C@H](CCCCCN*)C1] | (595) |
| 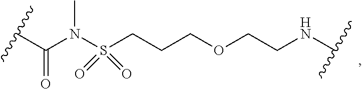 [SMILES: *NCCOCCCS(=O)(=O)N(C)C(*)=O] | (596) |
| 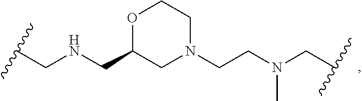 [SMILES: *CNC[C@@H]1CN(CCN(C)C*)CCO1] | (597) |
| 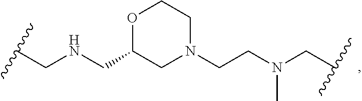 [SMILES: *CNC[C@H]1CN(CCN(C)C*)CCO1] | (598) |
| 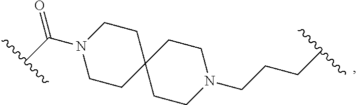 [SMILES: *CCCN1CCC2(CC1)CCN(C(*)=O)CC2] | (599) |
| 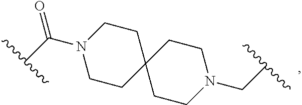 [SMILES: *CN1CCC2(CC1)CCN(C(*)=O)CC2] | (600) |
| 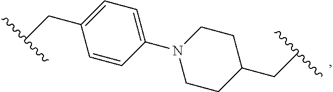 [SMILES: *Cc1ccc(N2CCC(C*)CC2)cc1] | (601) |
| 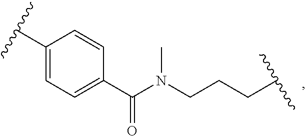 [SMILES: *CCCN(C)C(=O)c1ccc(*)cc1] | (602) |
| 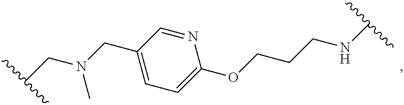 [SMILES: *CN(C)Cc1ccc(OCCCN*)nc1] | (603) |
| 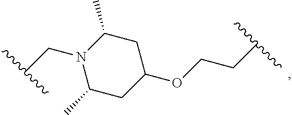 [SMILES: *CCOC1C[C@@H](C)N(C*)[C@@H](C)C1] | (604) |
| 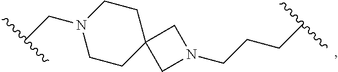 [SMILES: *CCCN1CC2(CCN(C*)CC2)C1] | (605) |
| 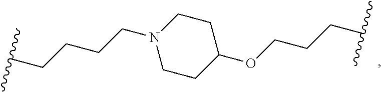 [SMILES: *CCCCN1CCC(OCCC*)CC1] | (606) |
| 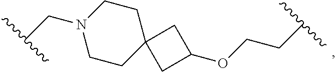 [SMILES: *CCOC1CC2(CCN(C*)CC2)C1] | (607) |
| 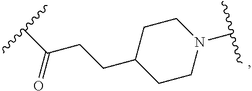 [SMILES: *C(=O)CCC1CCN(*)CC1] | (608) |
| 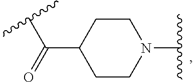 [SMILES: *C(=O)C1CCN(*)CC1] | (609) |
| 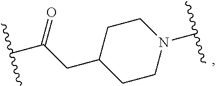 [SMILES: *C(=O)CC1CCN(*)CC1] | (610) |
| 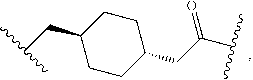 [SMILES: *C[C@H]1CC[C@H](CC(*)=O)CC1] | (611) |
| 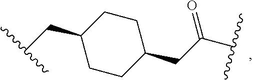 [SMILES: *C[C@H]1CC[C@@H](CC(*)=O)CC1] | (612) |
| 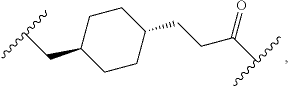 [SMILES: *C[C@H]1CC[C@H](CCC(*)=O)CC1] | (613) |
| 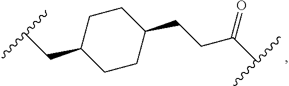 [SMILES: *C[C@H]1CC[C@@H](CCC(*)=O)CC1] | (614) |
| 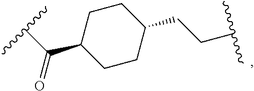 [SMILES: *CC[C@H]1CC[C@H](C(*)=O)CC1] | (615) |
| 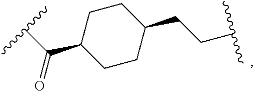 [SMILES: *CC[C@H]1CC[C@@H](C(*)=O)CC1] | (616) |
| 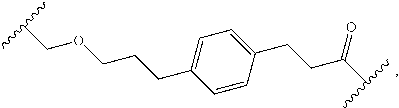 [SMILES: *COCCCc1ccc(CCC(*)=O)cc1] | (617) |
| 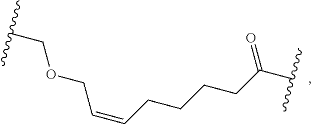 [SMILES: *COC/C=C\CCCCC(*)=O] | (618) |
| 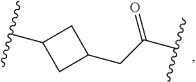 [SMILES: *C(=O)CC1CC(*)C1] | (619) |
| 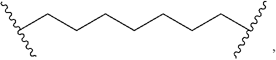 [SMILES: *CCCCCCC*] | (620) |
| 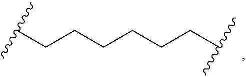 [SMILES: *CCCCCC*] | (621) |
| 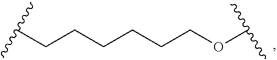 [SMILES: *CCCCCCO*] | (622) |
| 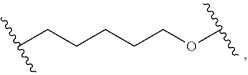 [SMILES: *CCCCCO*] | (623) |
| 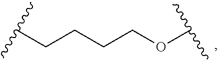 [SMILES: *CCCCO*] | (624) |
| 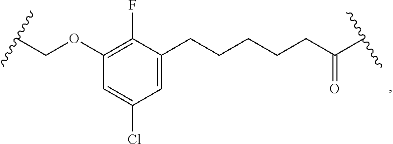 [SMILES: *COc1cc(Cl)cc(CCCCCC(*)=O)c1F] | (625) |
| 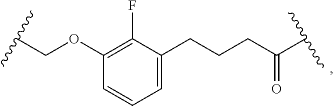 [SMILES: *COc1cccc(CCCC(*)=O)c1F] | (626) |
| 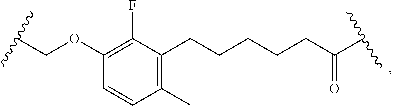 [SMILES: *COc1ccc(C)c(CCCCCC(*)=O)c1F] | (627) |
| 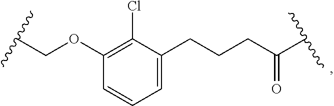 [SMILES: *COc1cccc(CCCC(*)=O)c1Cl] | (628) |
| 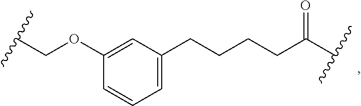 [SMILES: *COc1cccc(CCCCC(*)=O)c1] | (629) |
| 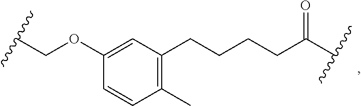 [SMILES: *COc1ccc(C)c(CCCCC(*)=O)c1] | (630) |
| 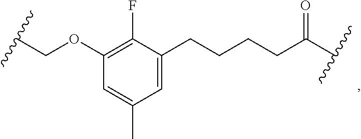 [SMILES: *COc1cc(C)cc(CCCCC(*)=O)c1F] | (631) |
| 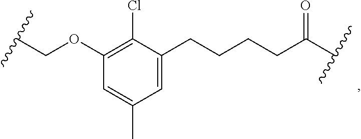 [SMILES: *COc1cc(C)cc(CCCCC(*)=O)c1Cl] | (632) |
| 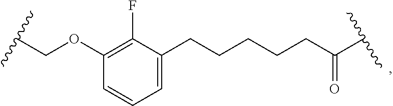 [SMILES: *COc1cccc(CCCCCC(*)=O)c1F] | (633) |
| 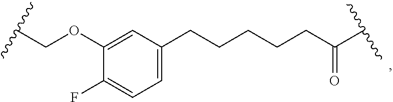 [SMILES: *COc1cc(CCCCCC(*)=O)ccc1F] | (634) |
| 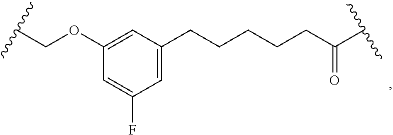 [SMILES: *COc1cc(F)cc(CCCCCC(*)=O)c1] | (635) |
| 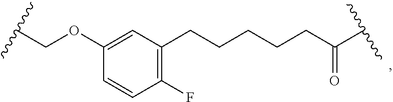 [SMILES: *COc1ccc(F)c(CCCCCC(*)=O)c1] | (636) |
| 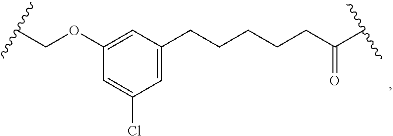 [SMILES: *COc1cc(Cl)cc(CCCCCC(*)=O)c1] | (637) |
| 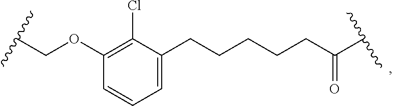 [SMILES: *COc1cccc(CCCCCC(*)=O)c1Cl] | (638) |
| 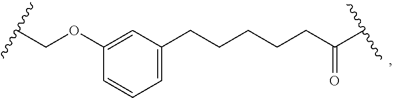 [SMILES: *COc1cccc(CCCCCC(*)=O)c1] | (639) |
| 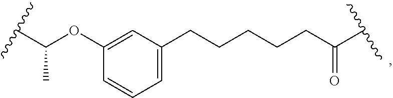 [SMILES: *C(=O)CCCCCc1cccc(O[C@@H](*)C)c1] | (640) |
| 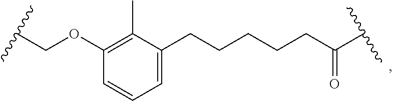 [SMILES: *COc1cccc(CCCCCC(*)=O)c1C] | (641) |
| 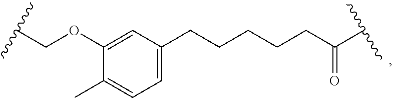 [SMILES: *COc1cc(CCCCCC(*)=O)ccc1C] | (642) |
| 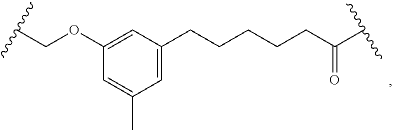 [SMILES: *COc1cc(C)cc(CCCCCC(*)=O)c1] | (643) |
| 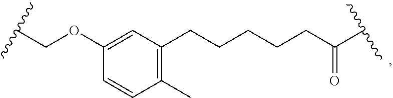 [SMILES: *COc1ccc(C)c(CCCCCC(*)=O)c1] | (644) |
| 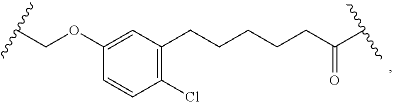 [SMILES: *COc1ccc(Cl)c(CCCCCC(*)=O)c1] | (645) |
| 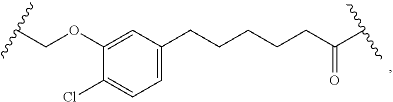 [SMILES: *COc1cc(CCCCCC(*)=O)ccc1Cl] | (646) |
| 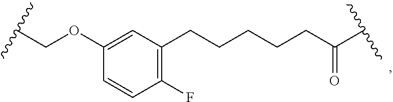 [SMILES: *COc1ccc(F)c(CCCCCC(*)=O)c1] | (647) |
| 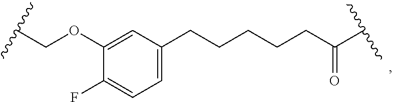 [SMILES: *COc1cc(CCCCCC(*)=O)ccc1F] | (648) |
| 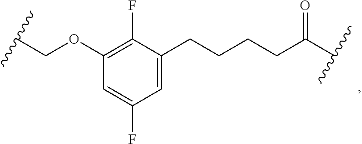 [SMILES: *COc1cc(F)cc(CCCCC(*)=O)c1F] | (649) |
| 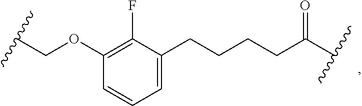 [SMILES: *COc1cccc(CCCCC(*)=O)c1F] | (650) |
| 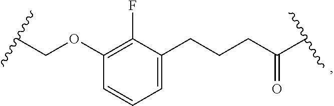 [SMILES: *COc1cccc(CCCC(*)=O)c1F] | (651) |
| 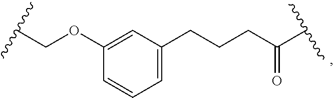 [SMILES: *COc1cccc(CCCC(*)=O)c1] | (652) |
| 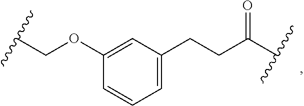 [SMILES: *COc1cccc(CCC(*)=O)c1] | (653) |
| 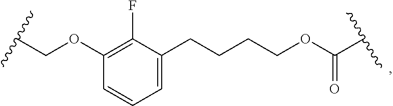 [SMILES: *COc1cccc(CCCCOC(*)=O)c1F] | (654) |
| 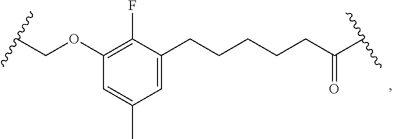 [SMILES: *COc1cc(C)cc(CCCCCC(*)=O)c1F] | (655) |
| 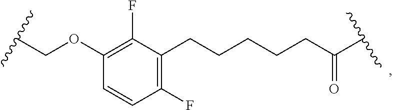 [SMILES: *COc1ccc(F)c(CCCCCC(*)=O)c1F] | (656) |
| 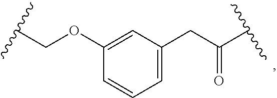 [SMILES: *COc1cccc(CC(*)=O)c1] | (657) |
| 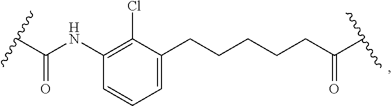 [SMILES: *C(=O)CCCCCc1cccc(NC(*)=O)c1Cl] | (658) |
| 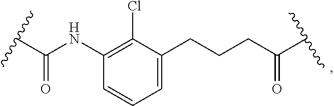 [SMILES: *C(=O)CCCc1cccc(NC(*)=O)c1Cl] | (659) |
| 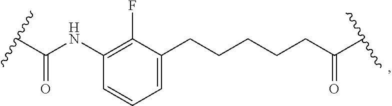 [SMILES: *C(=O)CCCCCc1cccc(NC(*)=O)c1F] | (660) |
| 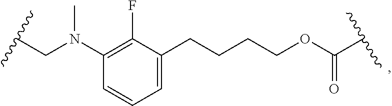 [SMILES: *CN(C)c1cccc(CCCCOC(*)=O)c1F] | (661) |
| 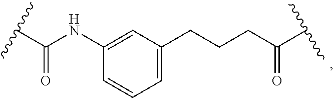 [SMILES: *C(=O)CCCc1cccc(NC(*)=O)c1] | (662) |
| 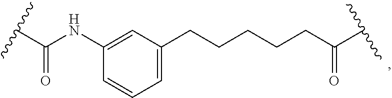 [SMILES: *C(=O)CCCCCc1cccc(NC(*)=O)c1] | (663) |
| 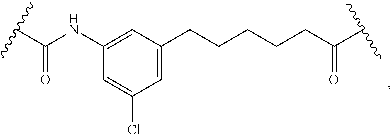 [SMILES: *C(=O)CCCCCc1cc(Cl)cc(NC(*)=O)c1] | (664) |
| 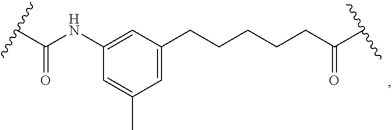 [SMILES: *C(=O)CCCCCc1cc(C)cc(NC(*)=O)c1] | (665) |
| 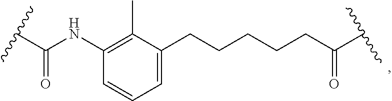 [SMILES: *C(=O)CCCCCc1cccc(NC(*)=O)c1C] | (666) |
| 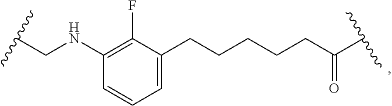 [SMILES: *CNc1cccc(CCCCCC(*)=O)c1F] | (667) |
| 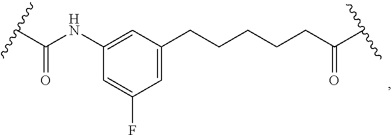 [SMILES: *C(=O)CCCCCc1cc(F)cc(NC(*)=O)c1] | (668) |
| 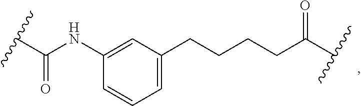 [SMILES: *C(=O)CCCCc1cccc(NC(*)=O)c1] | (669) |
| 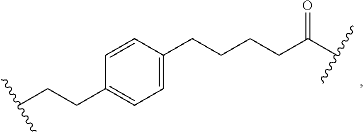 [SMILES: *CCc1ccc(CCCCC(*)=O)cc1] | (670) |
| 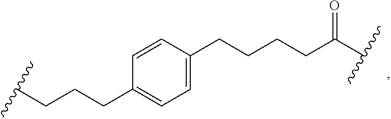 [SMILES: *CCCc1ccc(CCCCC(*)=O)cc1] | (671) |
| 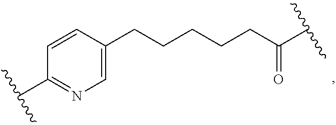 [SMILES: *C(=O)CCCCCc1ccc(*)nc1] | (672) |
| 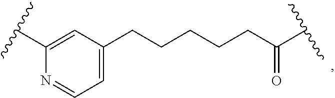 [SMILES: *C(=O)CCCCCc1ccnc(*)c1] | (673) |
| 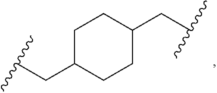 [SMILES: *CC1CCC(C*)CC1] | (674) |
| 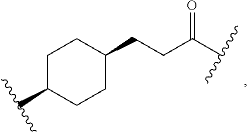 [SMILES: *C(=O)CC[C@H]1CC[C@@H](*)CC1] | (675) |
| 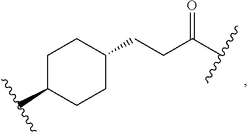 [SMILES: *C(=O)CC[C@H]1CC[C@H](*)CC1] | (676) |
| 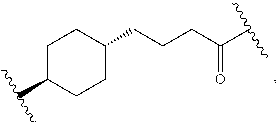 [SMILES: *C(=O)CCC[C@H]1CC[C@H](*)CC1] | (677) |
| 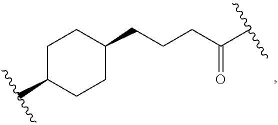 [SMILES: *C(=O)CCC[C@H]1CC[C@@H](*)CC1] | (678) |
| 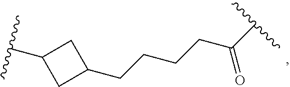 [SMILES: *C(=O)CCCCC1CC(*)C1] | (679) |
| 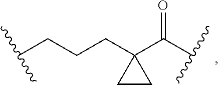 [SMILES: *CCCC1(C(*)=O)CC1] or any one of the linkers described in the linker section above | (680) |

In some embodiments, the present invention provides a compound having CBM described and disclosed herein, LBM set forth in Table A above, and a linker set forth in Table B above, or a pharmaceutically acceptable salt thereof.

Exemplary compounds of the invention are set forth in Table 1, below.

Lengthy table referenced here
US20240173419A1-20240530-T00001
Please refer to the end of the specification for access instructions.

In some embodiments, the present invention provides a compound set forth in Table 1, above, or a pharmaceutically acceptable salt thereof.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of each of which is herein incorporated by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In the schemes below, where a provided compound is formed having a reactive moiety (e.g., amine, alcohol, etc.), it is not shown but it is generally appreciated and well known by those having ordinary skill in the art that the reactivity of said reactive moiety may be masked by employing a suitable protecting group that can thereafter be removed in situ or during a separate synthetic step.

In certain embodiments, compounds of the present invention are generally prepared according to Scheme 1 set forth below:

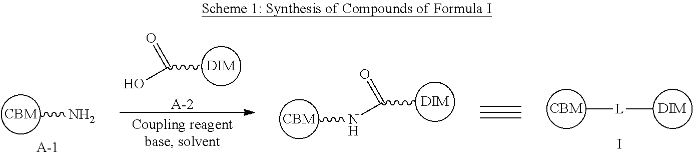

As depicted in Scheme 1, above, amine A-1 is coupled to acid A-2 using the a coupling reagent in the presence of the base (e.g., DIPEA) in a solvent (e.g., DMF) to form a compound of formula I with a linker comprising an amide bond. The squiggly bond, [symbol] , represents the portion of the linker between CBM and the terminal amino group of A-1 or the portion of the linker between DIM and the terminal carboxyl group of A-2, respectively. The amide bond can be formed using coupling reagents known in the art such as, but not limited to DCC, DIC, EDC, HATU, HBTU, HCTU, PyAOP, PyBOP, PyBrOP, BOP, BOP—Cl, DEPBT, T3P, TATU, TBTU, TNTU, TOTU, TPTU, TSTU, or TDBTU.

In certain embodiments, compounds of the present invention are generally prepared according to Scheme 2 set forth below:

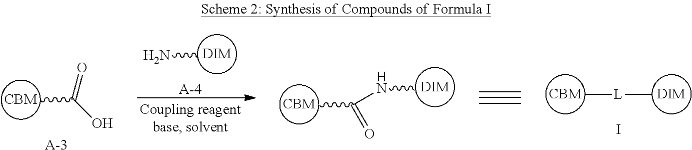

As depicted in Scheme 2, above, acid A-3 is coupled to amine A-4 using a coupling reagent in the presence of the base (e.g., DIPEA) in a solvent (e.g., DMF) to form a compound of formula I with a linker comprising an amide bond. The squiggly bond, [symbol] , represents the portion of the linker between CBM and the terminal carboxyl group of A-3 or the portion of the linker between DIM and the terminal amino group of A-4, respectively. The amide bond can be formed using coupling reagents known in the art such as, but not limited to DCC, DIC, EDC, HATU, HBTU, HCTU, PyAOP, PyBOP, PyBrOP, BOP, BOP—Cl, DEPBT, T3P, TATU, TBTU, TNTU, TOTU, TPTU, TSTU, or TDBTU.

In certain embodiments, compounds of the present invention are generally prepared according to Scheme 3 set forth below:

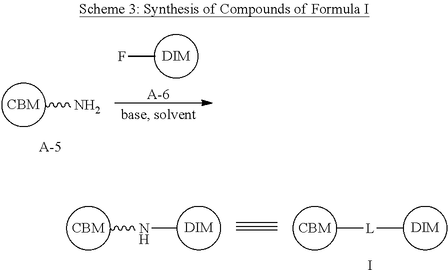

As depicted in Scheme 3, above, an S_{N}Ar displacement of fluoride A-6 by amine A-5 is effected in the presence of the base (e.g., DIPEA) in a solvent (e.g., DMF) to form a compound of formula I with a linker comprising a secondary amine. The squiggly bond, [symbol] , represents the portion of the linker between CBM and the terminal amino group of A-5.

In certain embodiments, compounds of the present invention are generally prepared according to Scheme 4 set forth below:

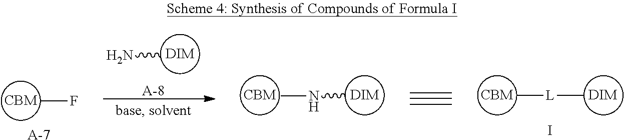

As depicted in Scheme 4, above, an S_{N}Ar displacement of fluoride A-7 by amine A-8 is effected in the presence of the base (e.g., DIPEA) in a solvent (e.g., DMF) to form a compound of formula I with a linker comprising a secondary amine. The squiggly bond, [symbol] , represents the portion of the linker between DIM and the terminal amino group of A-8.

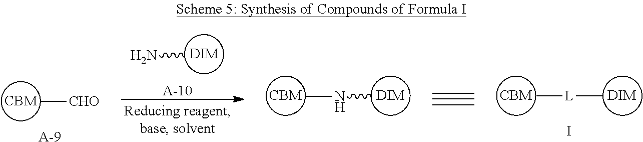

As depicted in Scheme 7, above, reductive amination of the mixture of aldehyde A-9 and amine A-10 is effected in the presence of a reducing agent (e.g., NaHB(OAc)₃) and base (e.g., KOAc) in a solvent (e.g., DMF/THF) to form a compound of formula I with a linker comprising a secondary amine. The squiggly bond, [symbol] , represents the portion of the linker between DIM and the terminal amino group of A-8.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the invention are described below in the Exemplification.

### 5. Uses, Formulation and Administration

Pharmaceutically acceptable compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that it is effective to measurably degrade and/or inhibit an CDK protein, or a mutant thereof, in a biological sample or in a patient. In certain embodiments, the amount of compound in compositions of this invention is such that it is effective to measurably degrade and/or inhibit an CDK protein, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily or degratorily active metabolite or residue thereof.

As used herein, the term “inhibitorily active metabolite or residue thereof” means that a metabolite or residue thereof is also an inhibitor of an CDK protein, or a mutant thereof.

As used herein, the term “degratorily active metabolite or residue thereof” means that a metabolite or residue thereof is also a degrader of an CDK protein, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

Compounds and compositions described herein are generally useful for the degradation and/or inhibition of kinase activity of one or more enzymes.

As used herein, the terms “CDK1-mediated”, “CDK2-mediated”, “CDK4-mediated”, “CDK6-mediated”, “CDK7-mediated”, “CDK8-mediated”, and/or “CDK9-mediated” disorders, diseases, and/or conditions as used herein means any disease or other deleterious condition in which one or more of CDK1, CDK2, CDK4, CDK6, CDK7, CDK8, and/or CDK9 or a mutant thereof, are known to play a role. Accordingly, another embodiment of the present invention relates to treating or lessening the severity of one or more diseases in which one or more of CDK1, CDK2, CDK4, CDK6, CDK7, CDK8, and/or CDK9 or a mutant thereof, are known to play a role.

Compounds of the present disclosure can degrade CDK2 or CDK2 and CCNE1 and therefore are useful for treating diseases wherein the underlying pathology is, wholly or partially, mediated by CDK2. Such diseases include cancer and other diseases with proliferation disorder. In some embodiments, the present disclosure provides treatment of an individual or a patient in vivo using a provided compound or a pharmaceutically acceptable salt thereof such that growth of cancerous tumors is inhibited. A provided compound or a pharmaceutically acceptable salt thereof can be used to inhibit the growth of cancerous tumors with aberrations that activate CDK2 activity. These include, but not limited to, disease (e.g., cancers) that are characterized by amplification or overexpression of CCNE1 such as ovarian cancer, uterine carcinosarcoma and breast cancer and p27 inactivation such as breast cancer and melanomas. Accordingly, in some embodiments of the methods, the patient has been previously determined to have an amplification of the CCNE1 gene and/or an expression level of CCNE1 in a biological sample obtained from the human subject that is higher than a control expression level of CCNE1. Alternatively, a provided compound or a pharmaceutically acceptable salt thereof can be used in conjunction with other agents or standard cancer treatments, as described below. In one embodiment, the present disclosure provides a method for inhibiting growth of tumor cells in vitro. The method includes contacting the tumor cells in vitro with a provided compound or a pharmaceutically acceptable salt thereof. In another embodiment, the present disclosure provides a method for inhibiting growth of tumor cells with CCNE1 amplification and overexpression in an individual or a patient. The method includes administering to the individual or patient in need thereof a therapeutically effective amount of a provided compound or a pharmaceutically acceptable salt thereof.

In some embodiments, provided herein is a method of inhibiting CDK2, comprising contacting the CDK2 with a provided compound or a pharmaceutically acceptable salt thereof. In some embodiments, provided herein is a method of inhibiting CDK2 in a patient, comprising administering to the patient a provided compound or a pharmaceutically acceptable salt thereof.

In some embodiments, provided herein is a method of degrading CDK2, comprising contacting the CDK2 with a provided compound or a pharmaceutically acceptable salt thereof. In some embodiments, provided herein is a method of degrading CDK2 in a patient, comprising administering to the patient a provided compound or a pharmaceutically acceptable salt thereof.

In some embodiments, provided herein is a method of degrading CDK2 and CCNE1, comprising contacting the CDK2 and CCNE1 with a provided compound or a pharmaceutically acceptable salt thereof. In some embodiments, provided herein is a method of degrading CDK2 and CCNE1 in a patient, comprising administering to the patient a provided compound or a pharmaceutically acceptable salt thereof.

In some embodiments, provided herein is a method for treating cancer. The method includes administering to a patient (in need thereof), a therapeutically effective amount of a provided compound or a pharmaceutically acceptable salt thereof. In another embodiment, the cancer is characterized by amplification or overexpression of CCNE1. In some embodiments, the cancer is ovarian cancer or breast cancer, characterized by amplification or overexpression of CCNE1.

In some embodiments, provided herein is a method of treating a disease or disorder associated with CDK2 in a patient, comprising administering to the patient a therapeutically effective amount of a provided compound or a pharmaceutically acceptable salt thereof. In some embodiments, the disease or disorder associated with CDK2 is associated with an amplification of the CCNE1 gene and/or overexpression of CCNE1.

In some embodiments, the disease or disorder associated with CDK2 is N-myc amplified neuroblastoma cells (see Molenaar et al., Proc. Natl. Acad. Sci. USA, 2009, 106(31):12968-12973), K-Ras mutant lung cancers (see Hu, S., et al., Mol. Cancer Ther., 2015, 14(11):2576-85), and cancers with FBW7 mutation and CCNE1 overexpression (see Takada et al., Cancer Res., 2017, 77(18):4881-4893).

In some embodiments, the disease or disorder associated with CDK2 is lung squamous cell carcinoma, lung adenocarcinoma, pancreatic adenocarcinoma, breast invasive carcinoma, uterine carcinosarcoma, ovarian serous cystadenocarcinoma, stomach adenocarcinoma, esophageal carcinoma, bladder urothelial carcinoma, mesothelioma, or sarcoma.

In some embodiments, the disease or disorder associated with CDK2 is lung adenocarcinoma, breast invasive carcinoma, uterine carcinosarcoma, ovarian serous cystadenocarcinoma, or stomach adenocarcinoma.

In some embodiments, the disease or disorder associated with CDK2 is an adenocarcinoma, carcinoma, or cystadenocarcinoma.

In some embodiments, the disease or disorder associated with CDK2 is uterine cancer, ovarian cancer, stomach cancer, esophageal cancer, lung cancer, bladder cancer, pancreatic cancer, or breast cancer.

In some embodiments, the disease or disorder associated with CDK2 is a cancer.

In some embodiments, the cancer is characterized by amplification or overexpression of CCNE1. In some embodiments, the cancer is ovarian cancer or breast cancer, characterized by amplification or overexpression of CCNE1.

In some embodiments, the breast cancer is chemotherapy or radiotherapy resistant breast cancer, endocrine resistant breast cancer, trastuzumab resistant breast cancer, or breast cancer demonstrating primary or acquired resistance to CDK4/6 inhibition. In some embodiments, the breast cancer is advanced or metastatic breast cancer.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include melanoma (e.g., metastatic malignant melanoma, BRAF and HSP90 inhibition-resistant melanoma), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer, urothelial cancer (e.g., bladder) and cancers with high microsatellite instability (MSIhigh). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma, Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, the compounds of the present disclosure can be used to treat sickle cell disease and sickle cell anemia.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma.

Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma, sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma, glioblastoma multiform, oligodendroglioma, schwannoma, retinoblastoma, congenital tumors), and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

It is believed that a provided compound or a pharmaceutically acceptable salt thereof may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

Co-Administration with One or More Other Therapeutic Agent(s)

Depending upon the particular condition, or disease, to be treated, additional therapeutic agents that are normally administered to treat that condition, can also be present in the compositions of this invention. As used herein, additional therapeutic agents that are normally administered to treat a particular disease, or condition, are known as “appropriate for the disease, or condition, being treated.”

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

A compound of the current invention can also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides, or in addition, be administered especially for tumor therapy in combination with chemotherapy, radiotherapy, immunotherapy, phototherapy, surgical intervention, or a combination of these. Long-term therapy is equally possible, as is adjuvant therapy in the context of other treatment strategies, as described above. Other possible treatments are therapy to maintain the patient's status after tumor regression, or even chemopreventive therapy, for example in patients at risk.

One or more other therapeutic agent(s) can be administered separately from a compound or composition of the invention, as part of a multiple dosage regimen. Alternatively, one or more other therapeutic agent(s) may be part of a single dosage form, mixed together with a compound of this invention in a single composition. If administered as a multiple dosage regime, one or more other therapeutic agent(s) and a compound or composition of the invention can be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent(s) and a compound or composition of the invention are administered as a multiple dosage regimen within greater than 24 hours apart.

As used herein, the term “combination,” “combined,” and related terms refers to the simultaneous or sequential administration of therapeutic agents in accordance with this invention. For example, a compound of the present invention can be administered with one or more other therapeutic agent(s) simultaneously or sequentially in separate unit dosage forms or together in a single unit dosage form. Accordingly, the present invention provides a single unit dosage form comprising a compound of the current invention, one or more other therapeutic agent(s), and a pharmaceutically acceptable carrier, adjuvant, or vehicle.

The amount of a compound of the invention and one or more other therapeutic agent(s) (in those compositions which comprise an additional therapeutic agent as described above) that can be combined with the carrier materials to produce a single dosage form varies depending upon the host treated and the particular mode of administration. Preferably, a composition of the invention should be formulated so that a dosage of between 0.01-100 mg/kg body weight/day of a compound of the invention can be administered.

In those compositions which comprise one or more other therapeutic agent(s), the one or more other therapeutic agent(s) and a compound of the invention can act synergistically. Therefore, the amount of the one or more other therapeutic agent(s) in such compositions may be less than that required in a monotherapy utilizing only that therapeutic agent. In such compositions a dosage of between 0.01-1,000 g/kg body weight/day of the one or more other therapeutic agent(s) can be administered.

The amount of one or more other therapeutic agent(s) present in the compositions of this invention may be no more than the amount that would normally be administered in a composition comprising that therapeutic agent as the only active agent. Preferably the amount of one or more other therapeutic agent(s) in the presently disclosed compositions ranges from about 50% to 100% of the amount normally present in a composition comprising that agent as the only therapeutically active agent. In some embodiments, one or more other therapeutic agent(s) is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is provided for dosing per the FDA label insert.

The compounds of this invention, or pharmaceutical compositions thereof, can also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

Exemplary Other Therapeutic Agents

In some embodiments, one or more other therapeutic agent is a Poly ADP ribose polymerase (PARP) inhibitor. In some embodiments, a PARP inhibitor is selected from olaparib (LYNPARZA®, AstraZeneca); rucaparib (RUBRACA®, Clovis Oncology); niraparib (ZEJULA®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (ZOLINZA®, Merck); romidepsin (ISTODAX®, Celgene); panobinostat (FARYDAK®, Novartis); belinostat (BELEODAQ®, Spectrum Pharmaceuticals); entinostat (SNDX-275, Syndax Pharmaceuticals) (NCT00866333); and chidamide (EPIDAZA®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (IBRANCE®, Pfizer); ribociclib (KISQALI®, Novartis); abemaciclib (Ly2835219, Eli Lilly); and trilaciclib (G1T28, G1 Therapeutics).

In some embodiments, one or more other therapeutic agent is a phosphatidylinositol 3 kinase (PI3K) inhibitor. In some embodiments, a PI3K inhibitor is selected from idelalisib (ZYDELIG®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

In some embodiments, one or more other therapeutic agent is a platinum-based therapeutic, also referred to as platins. Platins cause cross-linking of DNA, such that they inhibit DNA repair and/or DNA synthesis, mostly in rapidly reproducing cells, such as cancer cells. In some embodiments, a platinum-based therapeutic is selected from cisplatin (PLATINOL®, Bristol-Myers Squibb); carboplatin (PARAPLATIN®, Bristol-Myers Squibb; also, Teva; Pfizer); oxaliplatin (ELOXITIN® Sanofi-Aventis); nedaplatin (AQUPLA®, Shionogi), picoplatin (Poniard Pharmaceuticals); and satraplatin (JM-216, Agennix).

In some embodiments, one or more other therapeutic agent is a taxane compound, which causes disruption of microtubules, which are essential for cell division. In some embodiments, a taxane compound is selected from paclitaxel (TAXOL®, Bristol-Myers Squibb), docetaxel (TAXOTERE®, Sanofi-Aventis; DOCEFREZ®, Sun Pharmaceutical), albumin-bound paclitaxel (ABRAXANE®; Abraxis/Celgene), cabazitaxel (JEVTANA®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, YONDELIS®, Janssen Oncology), mechlorethamine (alkylating agent, VALCHLOR®, Aktelion Pharmaceuticals); vincristine (ONCOVIN®, Eli Lilly; VINCASAR®, Teva Pharmaceuticals; MARQIBO®, Talon Therapeutics); temozolomide (prodrug to alkylating agent 5-(3-methyltriazen-1-yl)-imidazole-4-carboxamide (MTIC) TEMODAR®, Merck); cytarabine injection (ara-C, antimetabolic cytidine analog, Pfizer); lomustine (alkylating agent, CEENU®, Bristol-Myers Squibb; GLEOSTINE®, NextSource Biotechnology); azacitidine (pyrimidine nucleoside analog of cytidine, VIDAZA®, Celgene); omacetaxine mepesuccinate (cephalotaxine ester) (protein synthesis inhibitor, SYNRIBO®; Teva Pharmaceuticals); asparaginase Erwinia chrysanthemi (enzyme for depletion of asparagine, ELSPAR®, Lundbeck; ERWINAZE®, EUSA Pharma); eribulin mesylate (microtubule inhibitor, tubulin-based antimitotic, HALAVEN®, Eisai); cabazitaxel (microtubule inhibitor, tubulin-based antimitotic, JEVTANA®, Sanofi-Aventis); capacetrine (thymidylate synthase inhibitor, XELODA®, Genentech); bendamustine (bifunctional mechlorethamine derivative, believed to form interstrand DNA cross-links, TREANDA®, Cephalon/Teva); ixabepilone (semi-synthetic analog of epothilone B, microtubule inhibitor, tubulin-based antimitotic, IXEMPRA®, Bristol-Myers Squibb); nelarabine (prodrug of deoxyguanosine analog, nucleoside metabolic inhibitor, ARRANON@, Novartis); clorafabine (prodrug of ribonucleotide reductase inhibitor, competitive inhibitor of deoxycytidine, CLOLAR®, Sanofi-Aventis); and trifluridine and tipiracil (thymidine-based nucleoside analog and thymidine phosphorylase inhibitor, LONSURF®, Taiho Oncology).

In some embodiments, one or more other therapeutic agent is a kinase inhibitor or VEGF-R antagonist. Approved VEGF inhibitors and kinase inhibitors useful in the present invention include: bevacizumab (AVASTIN®, Genentech/Roche) an anti-VEGF monoclonal antibody; ramucirumab (CYRAMZA®, Eli Lilly), an anti-VEGFR-2 antibody and ziv-aflibercept, also known as VEGF Trap (ZALTRAP®; Regeneron/Sanofi). VEGFR inhibitors, such as regorafenib (STIVARGA®, Bayer); vandetanib (CAPRELSA®, AstraZeneca); axitinib (INLYTA®, Pfizer); and lenvatinib (LENVIMA@, Eisai); Raf inhibitors, such as sorafenib (NEXAVAR®, Bayer AG and Onyx); dabrafenib (TAFINLAR@, Novartis); and vemurafenib (ZELBORAF®, Genentech/Roche); MEK inhibitors, such as cobimetanib (COTELLIC®, Exelexis/Genentech/Roche); trametinib (MEKINIST®, Novartis); Bcr-Abl tyrosine kinase inhibitors, such as imatinib (GLEEVEC®, Novartis); nilotinib (TASIGNA®, Novartis); dasatinib (SPRYCEL®, BristolMyersSquibb); bosutinib (BOSULIF®, Pfizer); and ponatinib (INCLUSIG®, Ariad Pharmaceuticals); Her2 and EGFR inhibitors, such as gefitinib (IRESSA®, AstraZeneca); erlotinib (TARCEEVA®, Genentech/Roche/Astellas); lapatinib (TYKERB®, Novartis); afatinib (GILOTRIF®, Boehringer Ingelheim); osimertinib (targeting activated EGFR, TAGRISSO®, AstraZeneca); and brigatinib (ALUNBRIG®, Ariad Pharmaceuticals); c-Met and VEGFR2 inhibitors, such as cabozanitib (COMETRIQ®, Exelexis); and multikinase inhibitors, such as sunitinib (SUTENT®, Pfizer); pazopanib (VOTRIENT®, Novartis); ALK inhibitors, such as crizotinib (XALKORI®, Pfizer); ceritinib (ZYKADIA®, Novartis); and alectinib (ALECENZa®, Genentech/Roche); Bruton's tyrosine kinase inhibitors, such as ibrutinib (IMBRUVICA®, Pharmacyclics/Janssen); and Flt3 receptor inhibitors, such as midostaurin (RYDAPT®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TK1258, Novartis); Chiauanib (Chipscreen Biosciences); CEP-11981 (Cephalon); linifanib (Abbott Laboratories); neratinib (HKI-272, Puma Biotechnology); radotinib (SUPECT®, IY5511, Il-Yang Pharmaceuticals, S. Korea); ruxolitinib (JAKAFI®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In some embodiments, one or more other therapeutic agent is an mTOR inhibitor, which inhibits cell proliferation, angiogenesis and glucose uptake. In some embodiments, an mTOR inhibitor is everolimus (AFINITOR®, Novartis); temsirolimus (TORISEL®, Pfizer); and sirolimus (RAPAMUNE®, Pfizer).

In some embodiments, one or more other therapeutic agent is a proteasome inhibitor. Approved proteasome inhibitors useful in the present invention include bortezomib (VELCADE®, Takeda); carfilzomib (KYPROLIS®, Amgen); and ixazomib (NINLARO®, Takeda).

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (LARTRUVO®; Eli Lilly). Approved EGFR antagonists which may be used in the present invention include cetuximab (ERBITUX®, Eli Lilly); necitumumab (PORTRAZZA®, Eli Lilly), panitumumab (VECTIBIX®, Amgen); and osimertinib (targeting activated EGFR, TAGRISSO®, AstraZeneca).

In some embodiments, one or more other therapeutic agent is an aromatase inhibitor. In some embodiments, an aromatase inhibitor is selected from exemestane (AROMASIN®, Pfizer); anastazole (ARIMIDEX®, AstraZeneca) and letrozole (FEMARA®, Novartis).

In some embodiments, one or more other therapeutic agent is an antagonist of the hedgehog pathway. Approved hedgehog pathway inhibitors which may be used in the present invention include sonidegib (ODOMZO®, Sun Pharmaceuticals); and vismodegib (ERIVEDGE®, Genentech), both for treatment of basal cell carcinoma.

In some embodiments, one or more other therapeutic agent is a folic acid inhibitor. Approved folic acid inhibitors useful in the present invention include pemetrexed (ALIMTA®, Eli Lilly).

In some embodiments, one or more other therapeutic agent is a CC chemokine receptor 4 (CCR4) inhibitor. CCR4 inhibitors being studied that may be useful in the present invention include mogamulizumab (POTELIGEO®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (RITUXAN®, Genentech/BiogenIdec); ofatumumab (anti-CD20, ARZERRA®, GlaxoSmithKline); obinutuzumab (anti-CD20, GAZYVA®, Genentech), ibritumomab (anti-CD20 and Yttrium-90, ZEVALIN®, Spectrum Pharmaceuticals); daratumumab (anti-CD38, DARZALEX®, Janssen Biotech), dinutuximab (anti-glycolipid GD2, UNITUXIN®, United Therapeutics); trastuzumab (anti-HER2, HERCEPTIN®, Genentech); ado-trastuzumab emtansine (anti-HER2, fused to emtansine, KADCYLA®, Genentech); and pertuzumab (anti-HER2, PERJETA®, Genentech); and brentuximab vedotin (anti-CD30-drug conjugate, ADCETRIS®, Seattle Genetics).

In some embodiments, one or more other therapeutic agent is a topoisomerase inhibitor. Approved topoisomerase inhibitors useful in the present invention include irinotecan (ONIVYDE®, Merrimack Pharmaceuticals); topotecan (HYCAMTIN®, GlaxoSmithKline). Topoisomerase inhibitors being studied which may be used in the present invention include pixantrone (PIXUVRI®, CTI Biopharma).

In some embodiments, one or more other therapeutic agent is an inhibitor of anti-apoptotic proteins, such as BCL-2. Approved anti-apoptotics which may be used in the present invention include venetoclax (VENCLEXTA®, AbbVie/Genentech); and blinatumomab (BLINCYTO®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (XTANDI®, Astellas/Medivation); approved inhibitors of androgen synthesis include abiraterone (ZYTIGA®, Centocor/Ortho); approved antagonist of gonadotropin-releasing hormone (GnRH) receptor (degaralix, FIRMAGON@, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (EVISTA®, Eli Lilly).

In some embodiments, one or more other therapeutic agent is an inhibitor of bone resorption. An approved therapeutic which inhibits bone resorption is Denosumab (XGEVA®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (ZOMETA®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFß). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGF-β trap compound (NCT02699515); and (NCT02517398). M7824 is comprised of a fully human IgG1 antibody against PD-L1 fused to the extracellular domain of human TGF-beta receptor II, which functions as a TGF-β“trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agents is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, TEMODAL CNF1010, CNF2024, CNF1010 from Conforma Therapeutics; temozolomide (TEMODAL®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name AROMASIN™. Formestane is marketed under the trade name LENTARON™. Fadrozole is marketed under the trade name AFEMA™. Anastrozole is marketed under the trade name ARIMIDEX™. Letrozole is marketed under the trade names FEMARA™ or FEMAr™. Aminoglutethimide is marketed under the trade name ORIMETEN™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name NOLVADEX™ Raloxifene hydrochloride is marketed under the trade name EVISTA™. Fulvestrant can be administered under the trade name FASLODEX™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (CASODEX™). The term “gonadorelin agonist” as used herein includes, but is not limited to abarelix, goserelin, and goserelin acetate. Goserelin can be administered under the trade name ZOLADEX™

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g., in the form as it is marketed, e.g., under the trademark CAMPTOSAR™. Topotecan is marketed under the trade name HYCAMPTIN™

The term “topoisomerase II inhibitor” as used herein includes, but is not limited to the anthracyclines such as doxorubicin (including liposomal formulation, such as CAELYX™), daunorubicin, epirubicin, idarubicin and nemorubicin, the anthraquinones mitoxantrone and losoxantrone, and the podophillotoxines etoposide and teniposide. Etoposide is marketed under the trade name ETOPOPHOS™ Teniposide is marketed under the trade name VM 26-Bristol Doxorubicin is marketed under the trade name ACRIBLASTIN™ or ADRIAMYCIN™. Epirubicin is marketed under the trade name FARMORUBICIN™. Idarubicin is marketed. under the trade name ZAVEDOS™. Mitoxantrone is marketed under the trade name NOVANTRON™

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name TAXOL™. Docetaxel is marketed under the trade name TAXOTERE™. Vinblastine sulfate is marketed under the trade name VINBLASTIN R.P™. Vincristine sulfate is marketed under the trade name FARMISTIN™

The term “alkylating agent” as used herein includes, but is not limited to, cyclophosphamide, ifosfamide, melphalan or nitrosourea (BCNU or Gliadel). Cyclophosphamide is marketed under the trade name CYCLOSTIN™. Ifosfamide is marketed under the trade name HOLOXAN™

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name XELODA™. Gemcitabine is marketed under the trade name GEMZAR™

The term “platin compound” as used herein includes, but is not limited to, carboplatin, cis-platin, cisplatinum and oxaliplatin. Carboplatin can be administered, e.g., in the form as it is marketed, e.g., under the trademark CARBOPLAT™. Oxaliplatin can be administered, e.g., in the form as it is marketed, e.g. under the trademark ELOXATIN™

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g., BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporne derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (GLEEVEC™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (HERCEPTIN™), cetuximab (ERBITUX™), Iressa, Tarceva, OSI-774, C1-1033, EKB-569, GW-2016, ELI, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, P13Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof, see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

Further examples of BTK inhibitory compounds, and conditions treatable by such compounds in combination with compounds of this invention can be found in WO2008039218 and WO2011090760, the entirety of which are incorporated herein by reference.

Further examples of SYK inhibitory compounds, and conditions treatable by such compounds in combination with compounds of this invention can be found in WO2003063794, WO2005007623, and WO2006078846, the entirety of which are incorporated herein by reference.

Further examples of PI3K inhibitory compounds, and conditions treatable by such compounds in combination with compounds of this invention can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds of this invention can be found in WO2009114512, WO2008109943, WO2007053452, WO2000142246, and WO2007070514, the entirety of which are incorporated herein by reference.

Further anti-angiogenic compounds include compounds having another mechanism for their activity, e.g., unrelated to protein or lipid kinase inhibition e.g., thalidomide (THALOMID™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (CELEBREX™), rofecoxib (VIOXX™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name DIDRONEL™. Clodronic acid is marketed under the trade name BONEFOS™ Tiludronic acid is marketed under the trade name Skelid™. Pamidronic acid is marketed under the trade name AREDIA™. Alendronic acid is marketed under the trade name FOSAMAX™. Ibandronic acid is marketed under the trade name BONDRANAT™. Risedronic acid is marketed under the trade name ACTONEL™. Zoledronic acid is marketed under the trade name ZOMETA™. The term “mTOR inhibitors” relates to compounds which inhibit the mammalian target of rapamycin (mTOR) and which possess antiproliferative activity such as sirolimus (RAPAMUNE®), everolimus (CERTICAN™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R¹¹⁵⁷⁷⁷ (ZARNESTRA™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (VELCADE™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g., hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-O-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (HERCEPTIN™), Trastuzumab-DM1, erbitux, bevacizumab (AVASTIN™), rituximab (RITUXAN®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]- amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; ANGIOSTATIN™; ENDOSTATIN™; anthranilic acid amides; ZD4190; Zd₆474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (AVASTIN™)

Photodynamic therapy as used herein refers to therapy which uses certain chemicals known as photosensitizing compounds to treat or prevent cancers. Examples of photodynamic therapy include treatment with compounds, such as VISUDYNE™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The structure of the active compounds identified by code numbers, generic or trade names may be taken from the actual edition of the standard compendium “The Merck Index” or from databases, e.g., Patents International (e.g., IMS World Publications).

Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer. An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIRI, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonist of activating receptors on NK cells. In some embodiments, an immuno-oncology agent is an antagonist of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete orinhibitTregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MED14736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Ikena Oncology, formerly known as Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (OPDIVO®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (POMALYST®, Celgene); lenalidomide (REVLIMID®, Celgene); ingenol mebutate (PICATO®, LEO Pharma).

In some embodiments, an immuno-oncology agent is a cancer vaccine. In some embodiments, the cancer vaccine is selected from sipuleucel-T (PROVENGE®, Dendreon/Valeant Pharmaceuticals), which has been approved for treatment of asymptomatic, or minimally symptomatic metastatic castrate-resistant (hormone-refractory) prostate cancer; and talimogene laherparepvec (IMLYGIC®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase-(TK-) deficient vaccinia virus engineered to express GM-CSF, for hepatocellular carcinoma (NCT02562755) and melanoma (NCT00429312); pelareorep (REOLYSIN®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-lh68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFa-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June et al.; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor y (RORyt). RORyt is a transcription factor with key roles in the differentiation and maintenance of Type 17 effector subsets of CD4+(Th17) and CD8+(Tc17) T cells, as well as the differentiation of IL-17 expressing innate immune cell subpopulations such as NK cells. In some embodiments, an activator of RORyt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that can be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORyt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams et al. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BITE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BITE®) antibody construct. In some embodiments, a bispecific T cell engager (BITE®) antibody construct is a CD19/CD3 bispecific antibody construct. In some embodiments, a bispecific T cell engager (BITE®) antibody construct is an EGFR/CD3 bispecific antibody construct. In some embodiments, a bispecific T cell engager (BITE®) antibody construct activates T cells. In some embodiments, a bispecific T cell engager (BITE®) antibody construct activates T cells, which release cytokines inducing upregulation of intercellular adhesion molecule 1 (ICAM-1) and FAS on bystander cells. In some embodiments, a bispecific T cell engager (BITE®) antibody construct activates T cells which result in induced bystander cell lysis. In some embodiments, the bystander cells are in solid tumors. In some embodiments, the bystander cells being lysed are in proximity to the BITE®-activated T cells. In some embodiments, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In some embodiments, the checkpoint inhibitor is a biologic therapeutic or a small molecule. In some embodiments, the checkpoint inhibitor is a monoclonal antibody, a humanized antibody, a fully human antibody, a fusion protein or a combination thereof. In some embodiments, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor is an immunostimulatory agent, a T cell growth factor, an interleukin, an antibody, a vaccine or a combination thereof. In some embodiments, the interleukin is IL-7 or IL-15. In some embodiments, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors can include small molecule inhibitors or can include antibodies, or antigen binding fragments thereof, that bind to and block or inhibit immune checkpoint receptors or antibodies that bind to and block or inhibit immune checkpoint receptor ligands. Illustrative checkpoint molecules that can be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include, but are not limited to, Tremelimumab (CTLA-4 blocking antibody), anti-OX40, PD-L1 monoclonal Antibody (Anti-B7-H1; MED14736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (OPDIVO®), ipilimumab (YERVOY@), and pembrolizumab (KEYTRUDA®). In some embodiments, the checkpoint inhibitor is selected from nivolumab (anti-PD-1 antibody, OPDIVO®, Bristol-Myers Squibb); pembrolizumab (anti-PD-1 antibody, KEYTRUDA®, Merck); ipilimumab (anti-CTLA-4 antibody, YERVOY®, Bristol-Myers Squibb); durvalumab (anti-PD-L1 antibody, IMFINZI®, AstraZeneca); and atezolizumab (anti-PD-L1 antibody, TECENTRIQ®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (KEYTRUDA®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (BAVENCIO®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that can be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that can be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981); and CTX-471 (Compass Therapeutics), an agonistic anti-CD137 antibody in metastatic or locally advanced malignancies (NCT03881488).

Checkpoint inhibitors that can be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that can be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that can be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that can be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that can be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that can be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that can be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that can be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that can be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### NUMBERED EMBODIMENTS

The following numbered embodiments, while non-limiting, are exemplary of certain aspects of the present disclosure:

Embodiment 1. A compound of formula I-a:

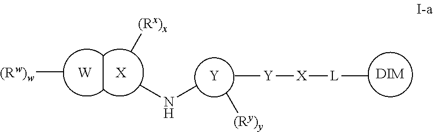

or a pharmaceutically acceptable salt thereof, wherein:
Ring W and Ring X are independently fused rings selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Ring Y is a ring selected from phenylenyl, a 4 to 7-membered saturated or partially unsaturated carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
Y is a covalent bond, —S(O)₂—, —S(O)—, —S(O)(NR)—, —P(O)R—, or —P(O)OR—;
X is —CR₂—, —CFR—, —CF₂—, —NR—, or an optionally substituted ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
each R^{w}, R^{x}, and R^{y} is independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂; or
   two R^{w} groups attached to the same carbon atom are optionally taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur;
w, x, and y are independently 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

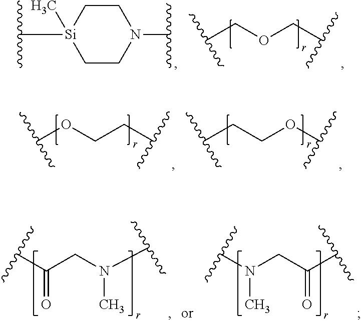

each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10; and
DIM is a degradation inducing moiety, such as a ligase binding moiety (LBM), lysine mimetic, or hydrogen atom.

Embodiment 2. A compound of formula I-b:

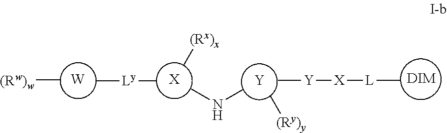

or a pharmaceutically acceptable salt thereof, wherein:
Ring W and Ring X are independently rings selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Ring Y is a ring selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Y is a covalent bond, —S(O)₂—, —S(O)—, —S(O)(NR)—, —P(O)R—, or —P(O)OR—;
X is —CR₂—, —CFR—, —CF₂—, —NR—, or an optionally substituted ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
each R^{w}, R^{x}, and R^{y} is independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂; or
two R^{w} groups attached to the same carbon atom are optionally taken together to form a spiro fused ring selected from a 3-5 membered saturated or partially unsaturated carbocyclyl and a 3-5 membered saturated or partially unsaturated heterocyclyl having 1-2 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
L^{y} is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CF₂—, —CRF—, —NR—, —S—, —S(O)—, —S(O)₂— or —CR═CR—; and
v is 0 or 1;
w, x, and y are independently 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

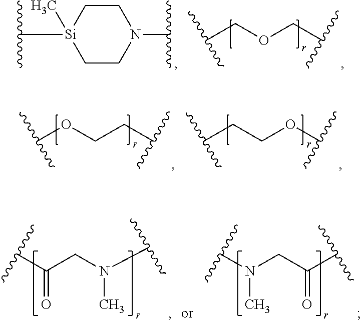

each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10; and
DIM is a degradation inducing moiety, such as a ligase binding moiety (LBM), lysine mimetic, or hydrogen atom.

Embodiment 3. The compound of either embodiment 1 or embodiment 2, wherein LBM is a cereblon E3 ubiquitin ligase binding moiety, a VHL E3 ubiquitin ligase binding moiety, an IAP E3 ubiquitin ligase binding moiety, or an MDM2 E3 ubiquitin ligase binding moiety.

Embodiment 4. The compound of embodiment 3, wherein LBM is an cereblon E3 ubiquitin ligase binding moiety and said compound is of formula I-nn-1:

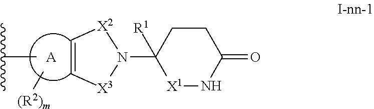

or a pharmaceutically acceptable salt thereof, wherein:
each of X¹, X², and X³ is independently a bivalent moiety selected from a covalent bond, —CH₂—, —C(O)—, —C(S)—, or

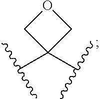 [SMILES: *C1(*)COC1]

R¹ is hydrogen, halogen, —CN, —OR, —SR, —(O)R, —S(O)₂R, —NR₂, or an optionally substituted C₁₋₄ aliphatic;
each of R² is independently hydrogen, R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
Ring A is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
m is 0, 1, 2, 3 or 4;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

Embodiment 5. The compound of embodiment 3, wherein LBM is a cereblon E3 ubiquitin ligase binding moiety and said compound is of formula I-aa:

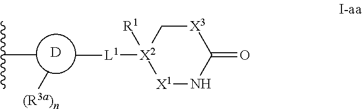

or a pharmaceutically acceptable salt thereof, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

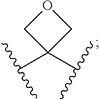 [SMILES: *C1(*)COC1]

X² is a carbon atom or silicon atom;
X³ is a bivalent moiety selected from —CR₂—, —NR—, —O—, —S—, or —SiR₂—;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur;
R¹ is hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
each R² is independently hydrogen, R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a bi- or tricyclic ring selected from

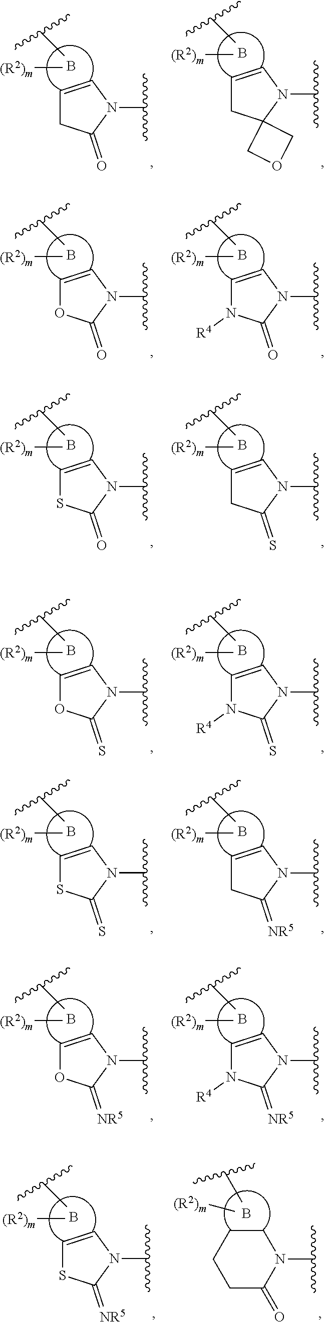

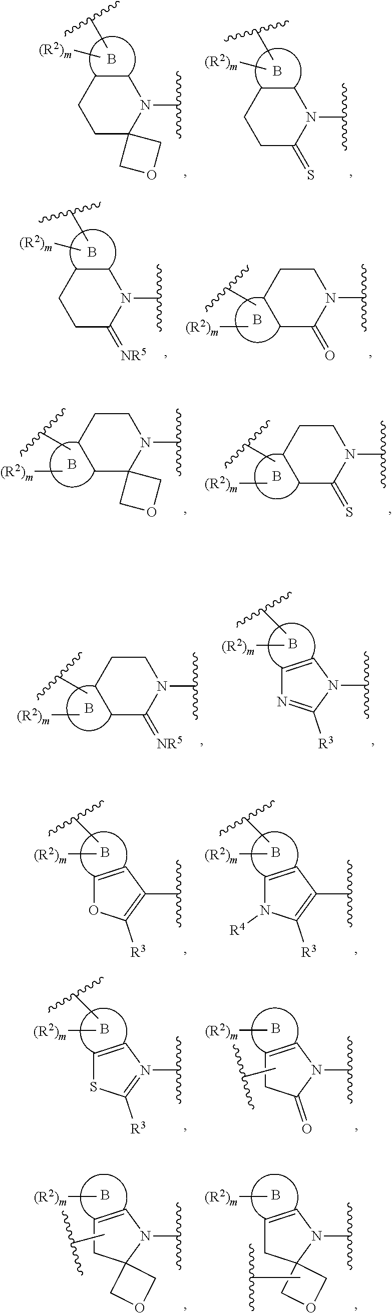

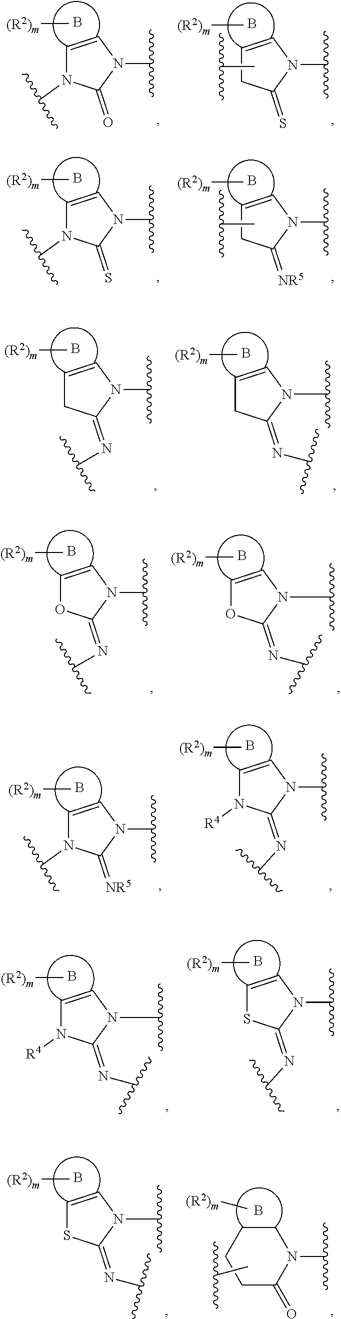

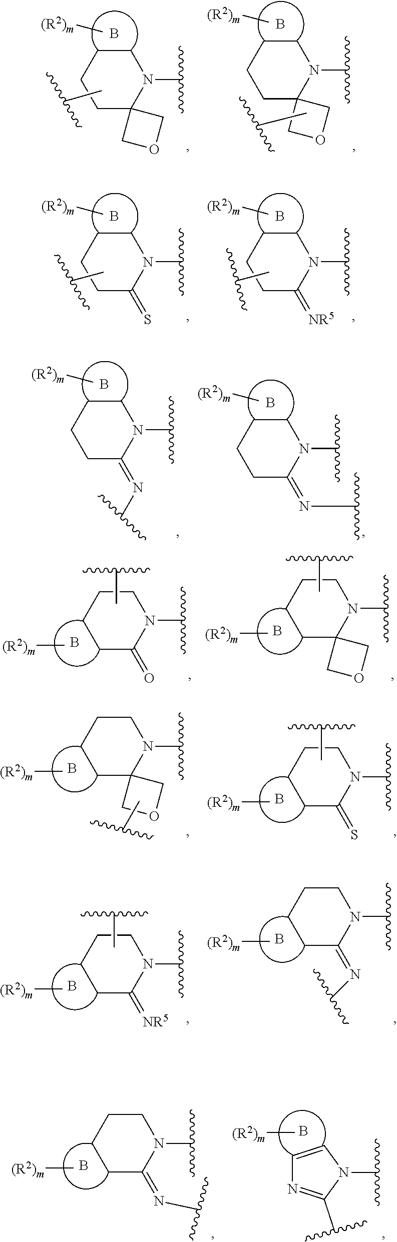

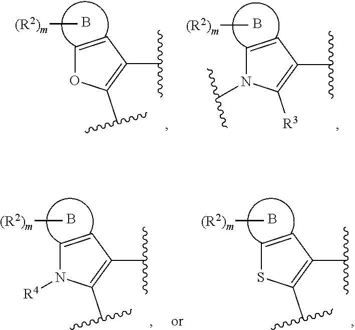

wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen or sulfur;
R³ is selected from hydrogen, halogen, —OR, —N(R)₂, or —SR;
each R⁴ is independently hydrogen, R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—; and
m is 0, 1, 2, 3 or 4.

Embodiment 6. The compound of embodiment 5, wherein said compound is a compound of any of the following formulae:

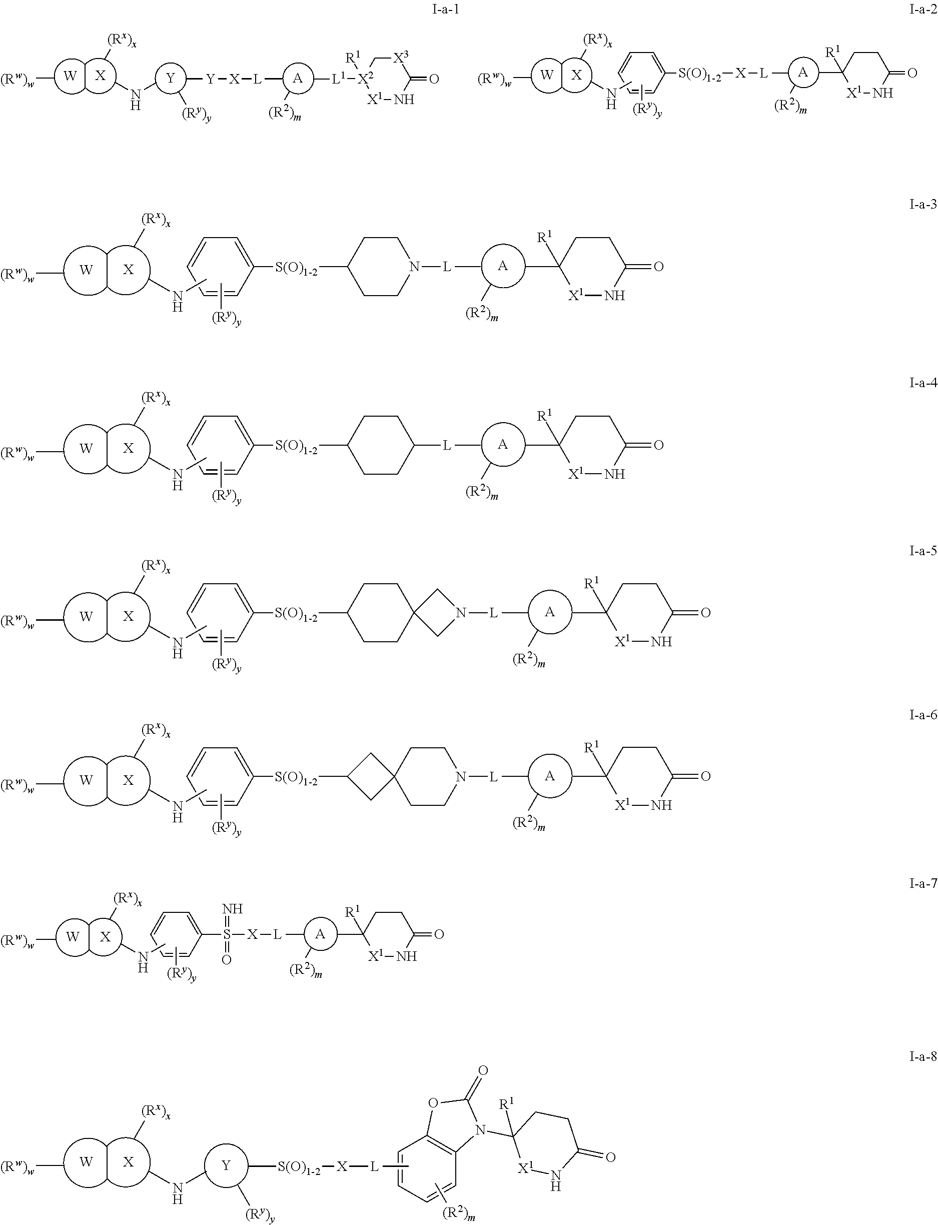

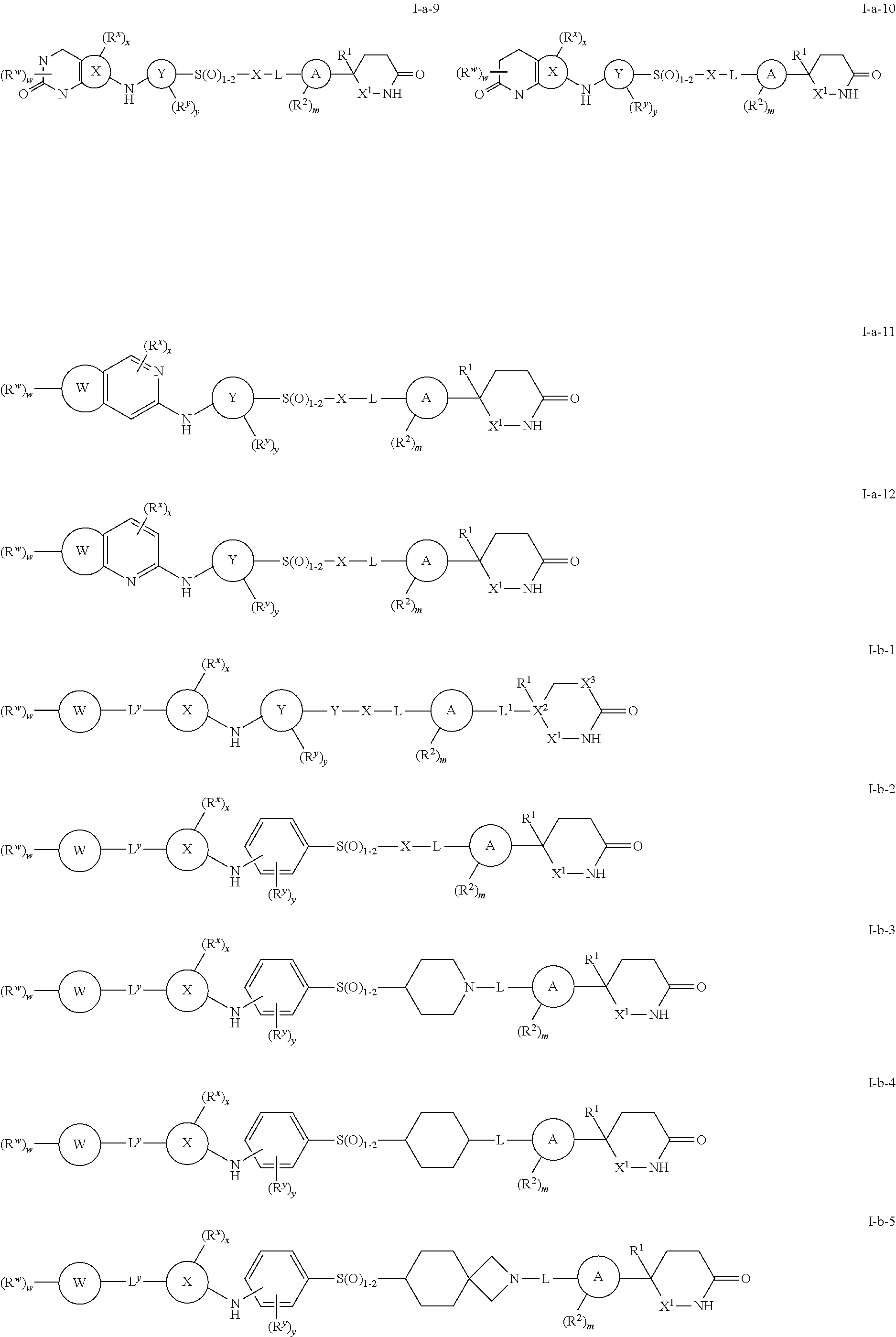

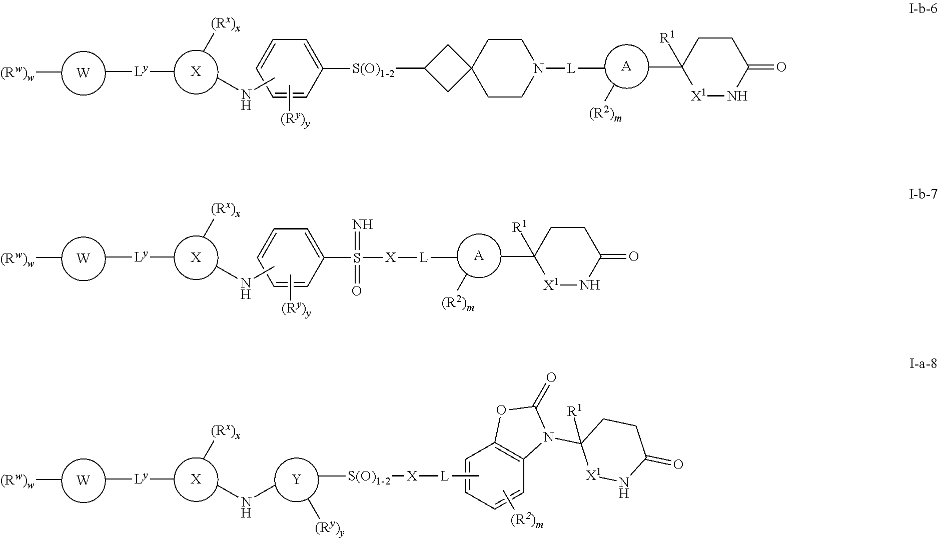

or pharmaceutically acceptable salt thereof.

Embodiment 7. The compound of embodiment 3, wherein LBM is a cereblon E3 ubiquitin ligase binding moiety and said compound is of formula I-nn:

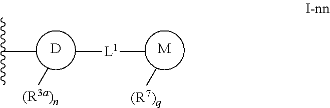

or a pharmaceutically acceptable salt thereof, wherein:
Ring M is selected from

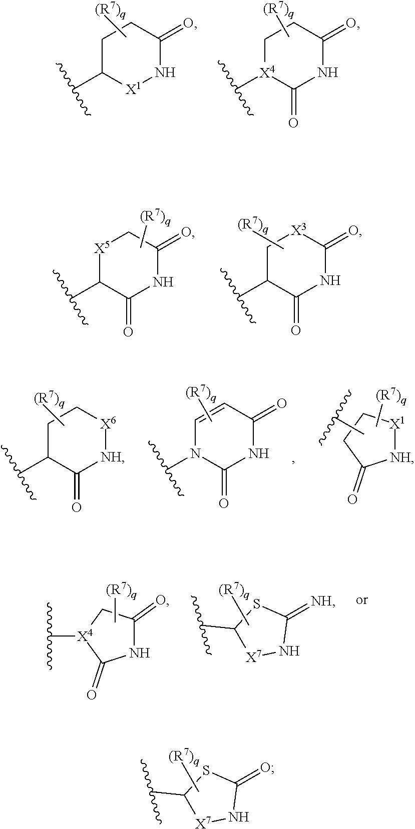

each of X¹, X⁶, and X⁷ is independently a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

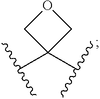 [SMILES: *C1(*)COC1]

each of X³ and X⁵ is independently a bivalent moiety selected from a covalent bond, —CR₂—, —NR—, —O—, —S—, or —SiR₂—;
X⁴ is a trivalent moiety selected from

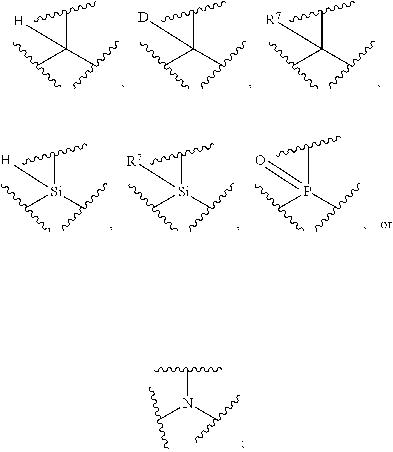

each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R^{3a} is independently hydrogen, R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁷ is independently hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic; or
R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, or sulfur;
   two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, or sulfur;
   two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, or sulfur, or a 7-13 membered saturated, partially unsaturated, bridged heterocyclic ring, or a spiro heterocyclic ring having 1-3 heteroatoms, independently selected from boron, nitrogen, oxygen, silicon, or sulfur;
Ring D is selected from 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
n is 0, 1, 2, 3, or 4; and
q is 0, 1, 2, 3, or 4.

Embodiment 8. The compound of embodiment 7, wherein said compound is a compound of any of the following formulae:

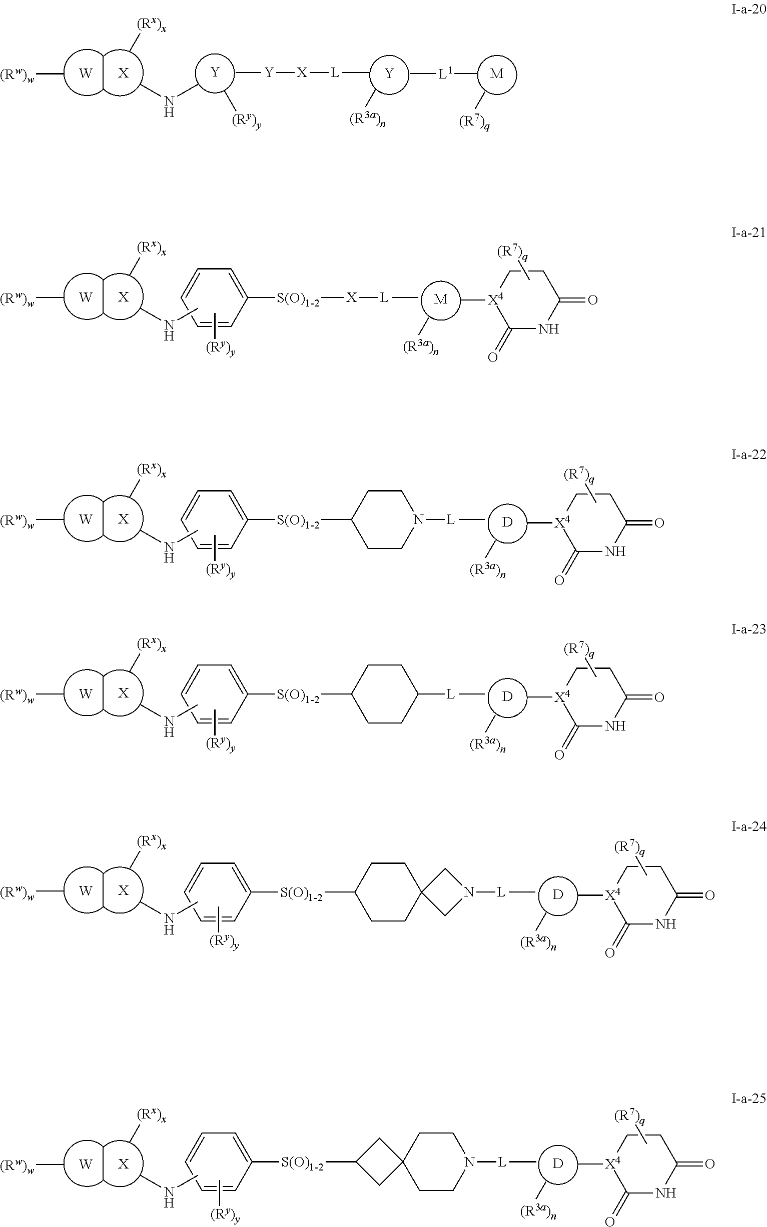

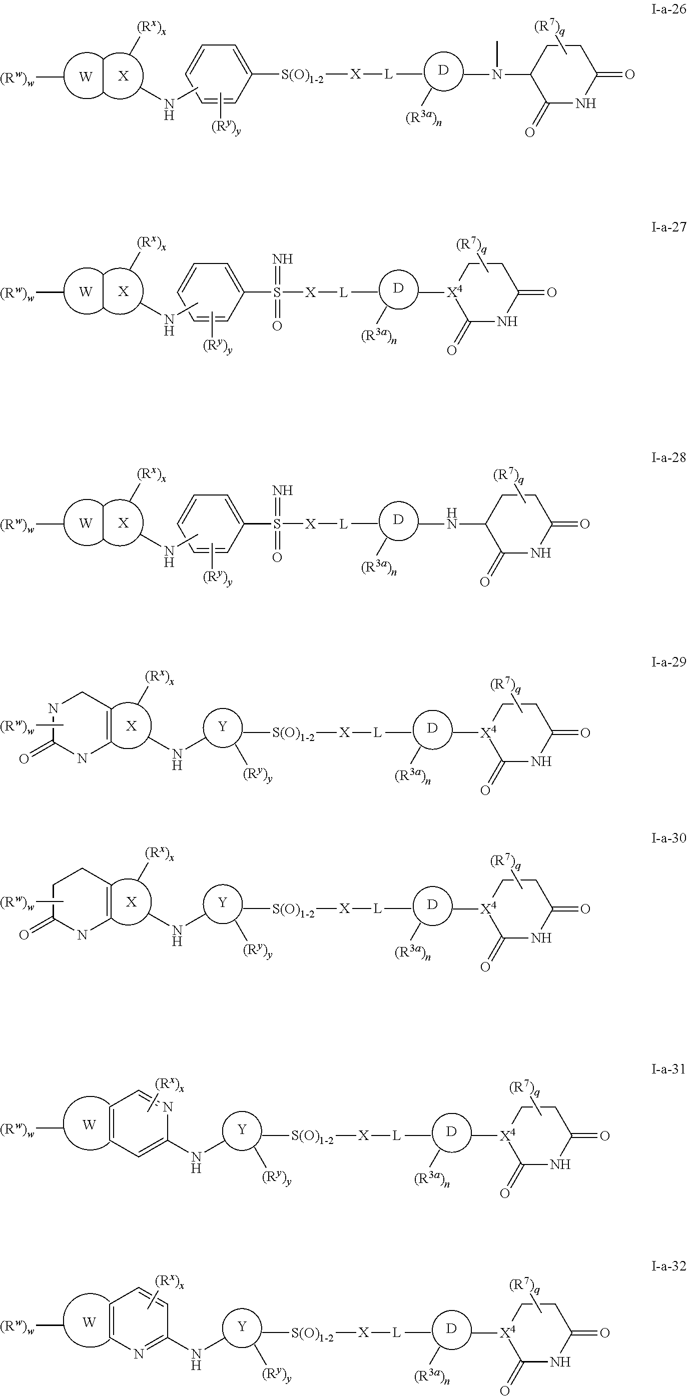

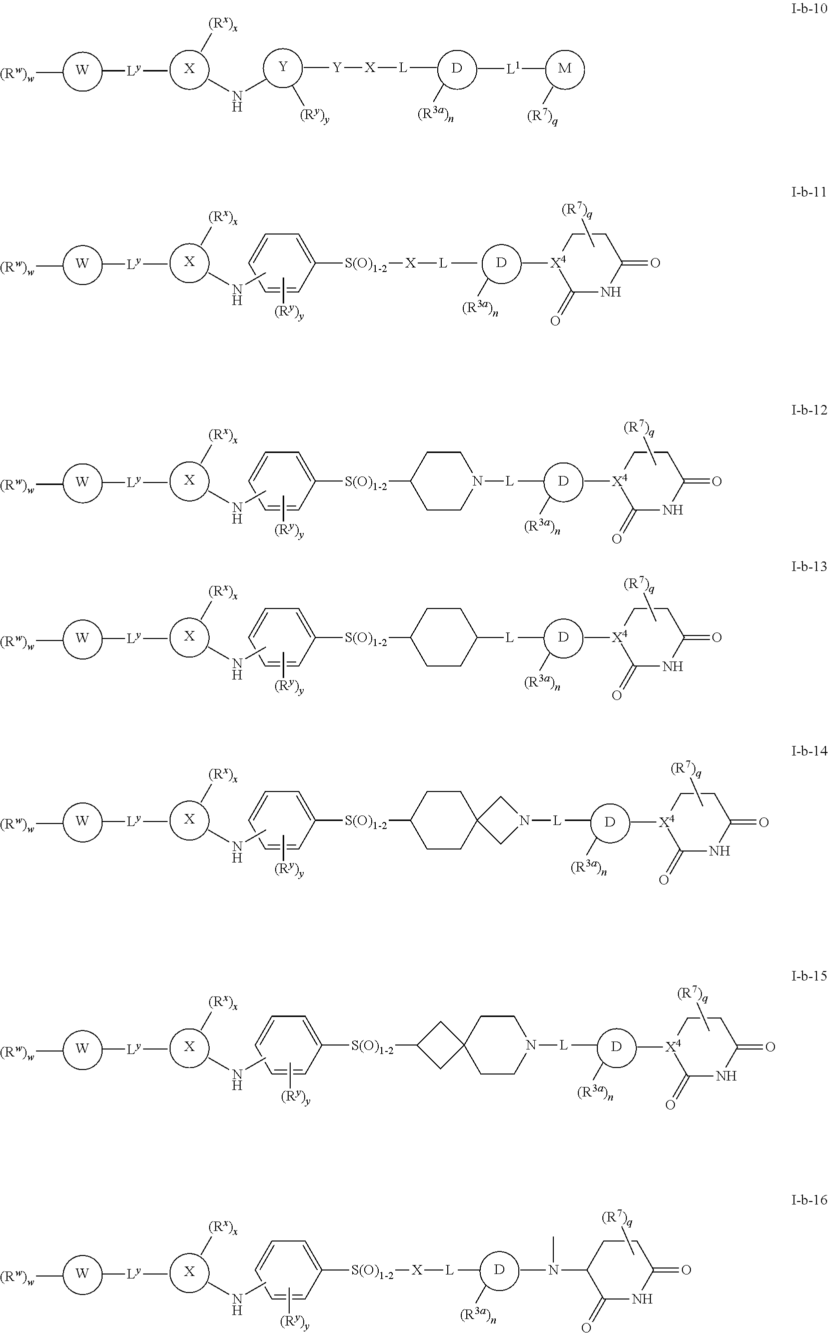

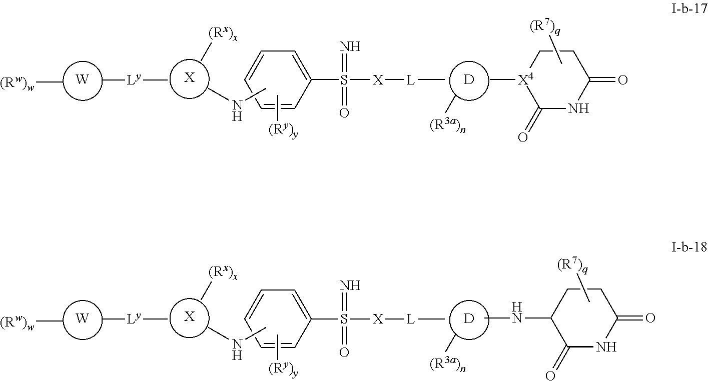

or pharmaceutically acceptable salt thereof.

Embodiment 9. The compound of embodiment 3, wherein LBM is a VHL E3 ubiquitin ligase binding moiety and said compound is selected from any of the following formulae:

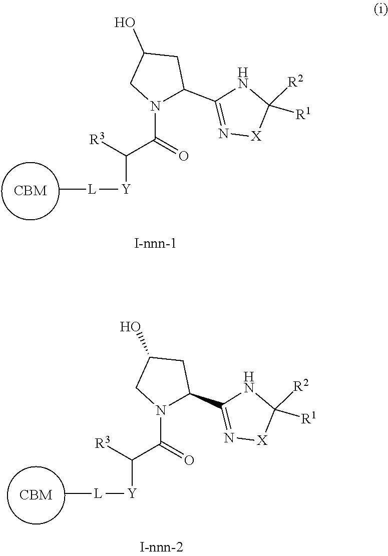

or a pharmaceutically acceptable salt thereof, wherein each of the variables R¹, R², R³, X, and Y is as defined and described in WO 2019/084026;

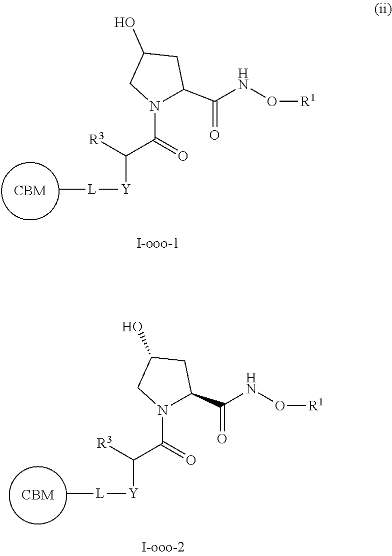

or a pharmaceutically acceptable salt thereof, wherein each of the variables R¹, R³, and Y is as defined and described in WO 2019/084030;

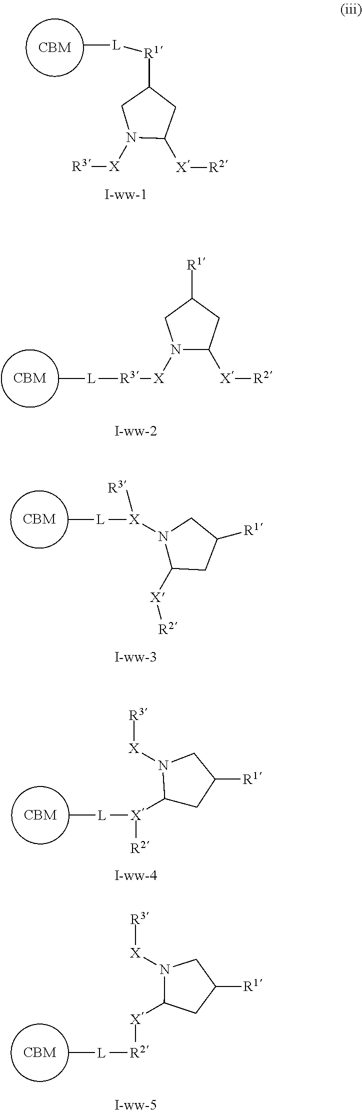

or a pharmaceutically acceptable salt thereof, wherein each of the variables R^{1′}, R^{2′}, R^{3′}, X, and X′ is as defined and described in WO 2013/106643 and US 2014/0356322;

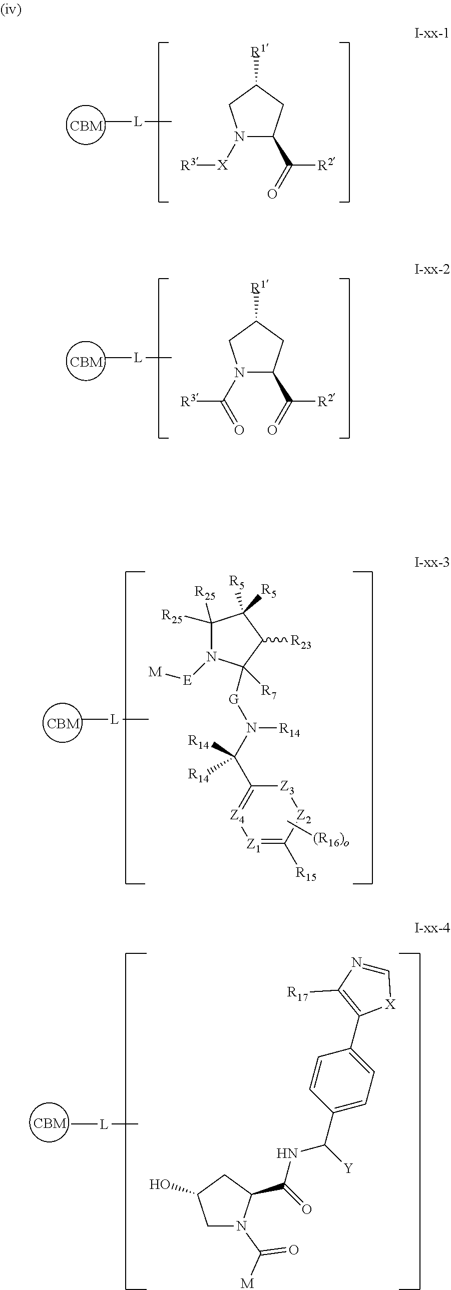

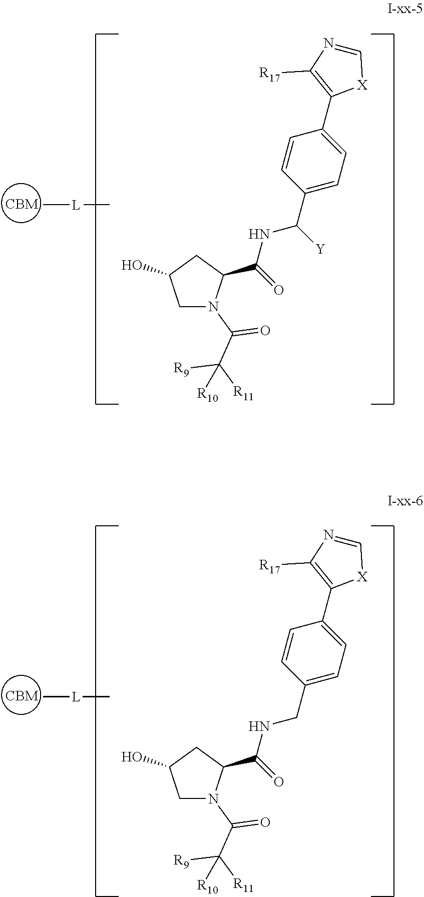

or a pharmaceutically acceptable salt thereof, wherein each of the variables R^{1′}, R^{2′}, R^{3′}, R₅, R₆, R₇, R₉, R₁₀, R₁₁, R₁₄, R₁₅, R₁₆, R₁₇, R₂₃, R₂₅, E, G, M, X, X′, Y, Z₁, Z₂, Z₃, Z₄, and o is as defined and described in WO 2016/149668 and US 2016/0272639; and

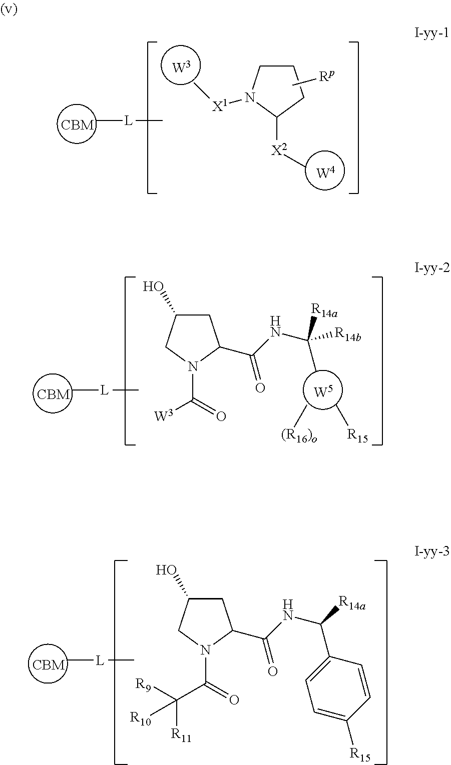

or a pharmaceutically acceptable salt thereof, wherein each of the variables RP, R₉, R₁₀, R₁₁, R₁₄ₐ, R_{14b}, R₁₅, R₁₆, W¹, W⁴, W⁵, X¹, X², and o is as defined and described in WO 2016/118666 and US 2016/0214972.

Embodiment 10. The compound according to either embodiment 3 or embodiment 9, wherein the VHL E3 ubiquitin ligase binding moiety is selected from

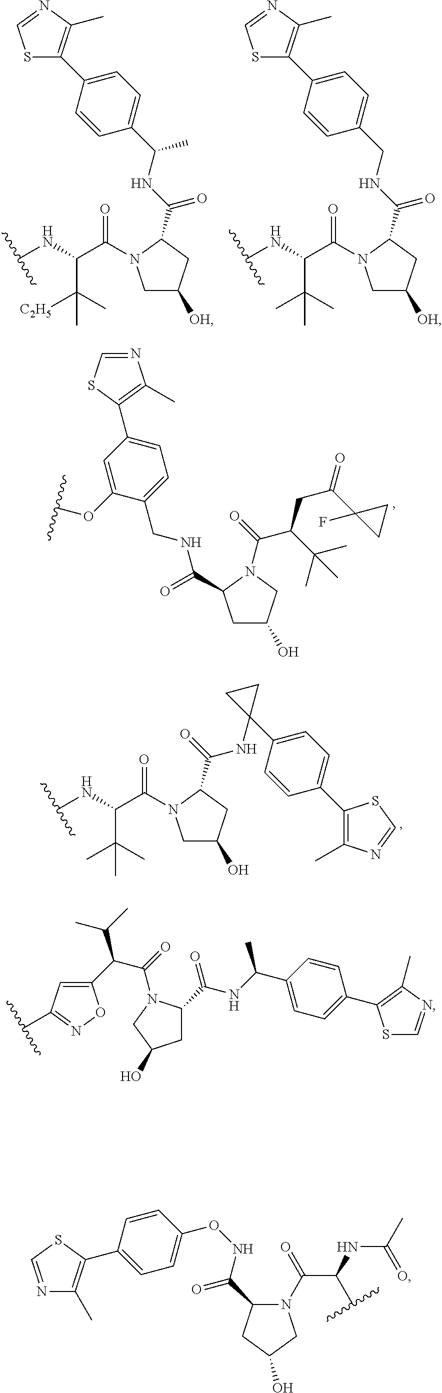

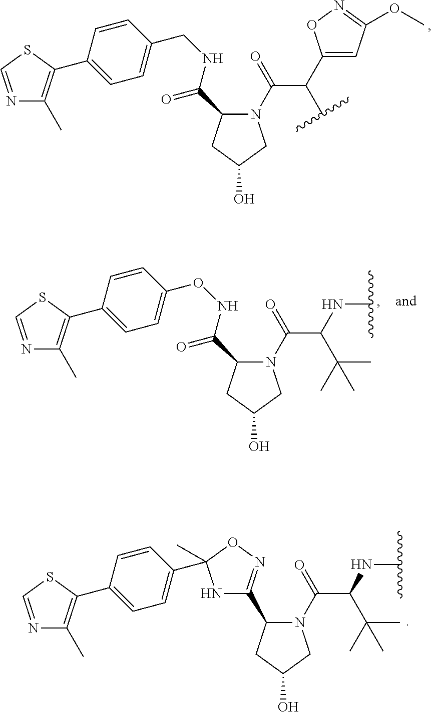 [SMILES: *C(C(=O)N1C[C@H](O)C[C@H]1C(=O)NCc1ccc(-c2scnc2C)cc1)c1cc(OC)no1 ; *NC(C(=O)N1C[C@H](O)C[C@H]1C(=O)NOc1ccc(-c2scnc2C)cc1)C(C)(C)C ; *N[C@H](C(=O)N1C[C@H](O)C[C@H]1C1=NOC(C)(c2ccc(-c3scnc3C)cc2)N1)C(C)(C)C]

Embodiment 11. The compound of embodiment 3, wherein LBM is a IAP E3 ubiquitin ligase binding moiety and said compound is selected from any one of the following formulae:

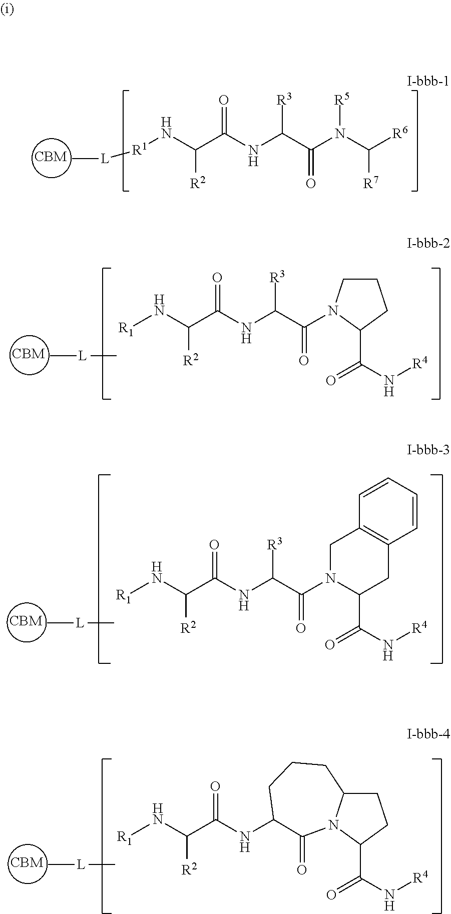

or a pharmaceutically acceptable salt thereof, wherein each of the variables R¹, R², R³, R⁴, R⁵, R⁶, and R⁷, is as defined and described in WO 2017/011590 and US 2007/037004; and

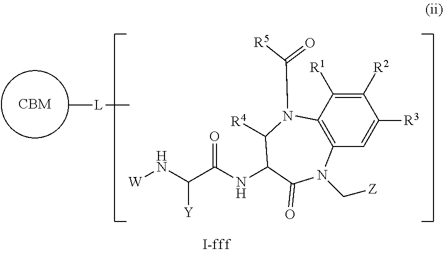

or a pharmaceutically acceptable salt thereof, wherein each of the variables W, Y, Z, R¹, R², R³, R⁴, and R is as described and defined in WO 2014/044622, US 2015/0225449. WO 2015/071393, and US 2016/0272596.

Embodiment 12. The compound according to embodiment 3 or embodiment 11, wherein the IAP E3 ubiquitin ligase binding moiety is selected from

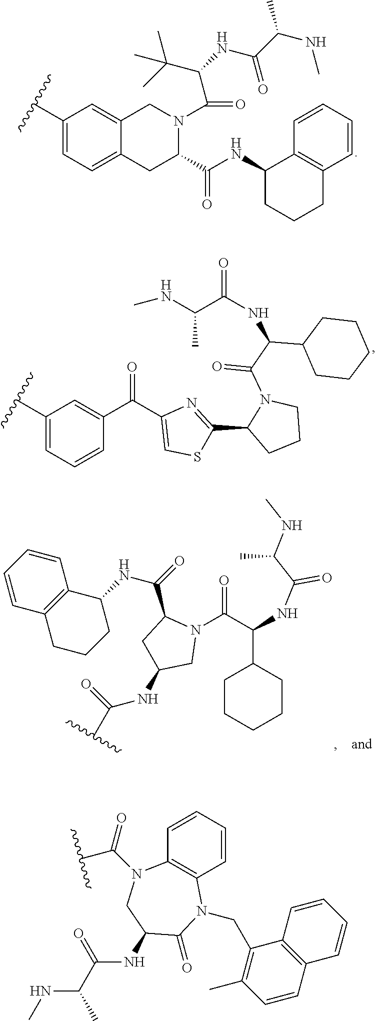

Embodiment 13. The compound of embodiment 3, wherein LBM is an MDM2 E3 ubiquitin ligase binding moiety and said compound is selected from any one of the following formulae:

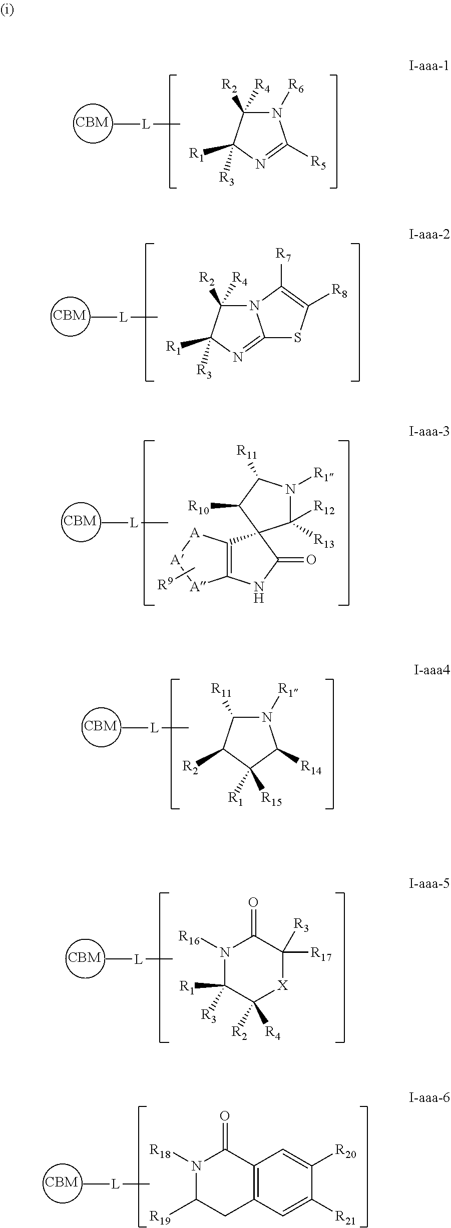

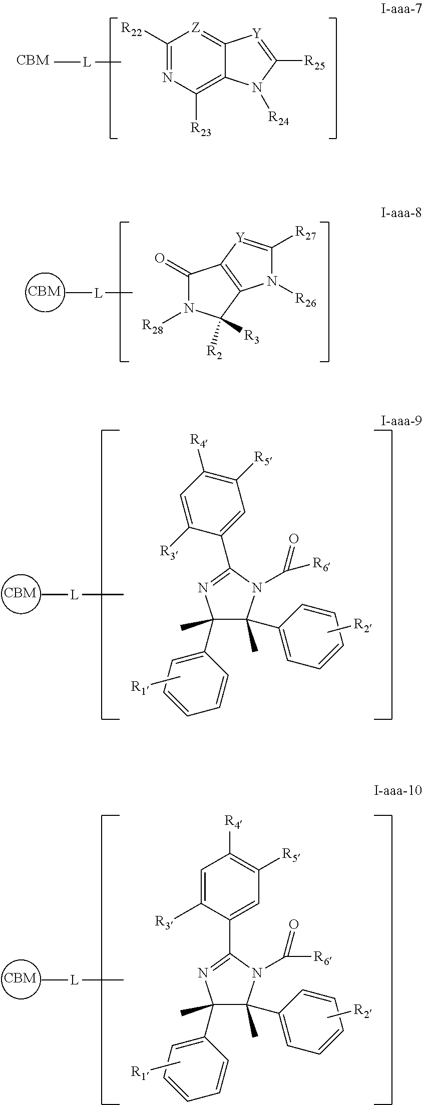

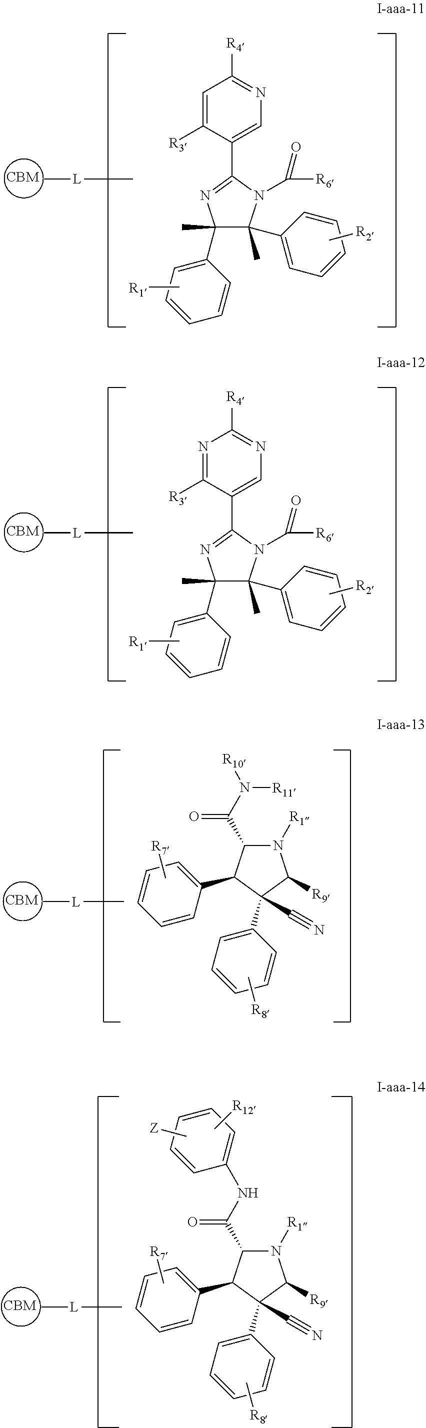

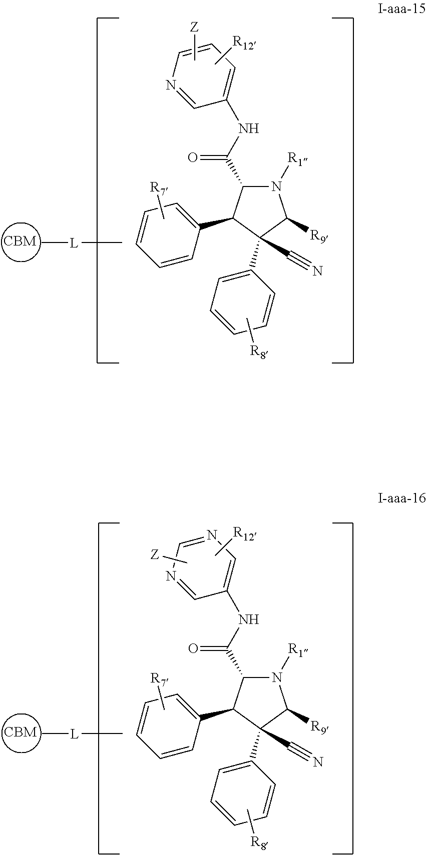

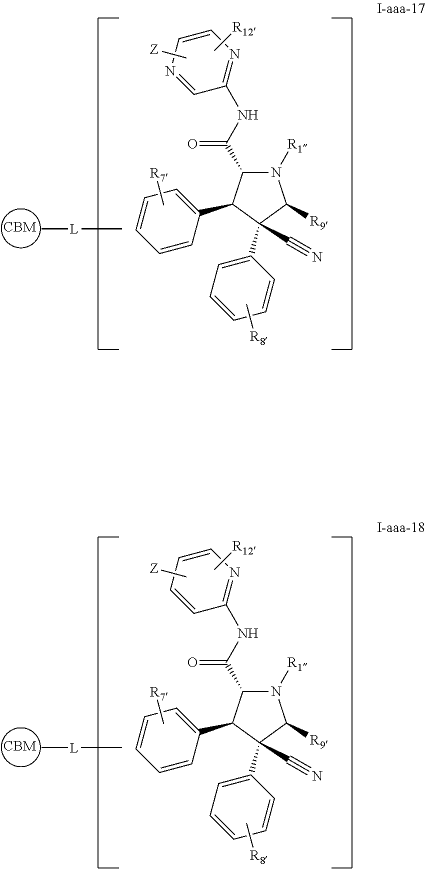

or a pharmaceutically acceptable salt thereof, wherein each of the variables R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂ₒ, R₂₁, R₂₂, R₂₃, R₂₄, R₂₅, R₂₆, R₂₇, R₂₈, R_{1′}, R_{2′}, R_{3′}, R_{4′}, R_{5′}, R_{6′}, R_{7′}, R_{8′}, R_{9′}, R_{10′}, R_{11′}, R_{12′}, R_{1″}, A, A′, A″, X, Y, and Z is as defined and described in WO 2017/011371 and US 2017/008904; and

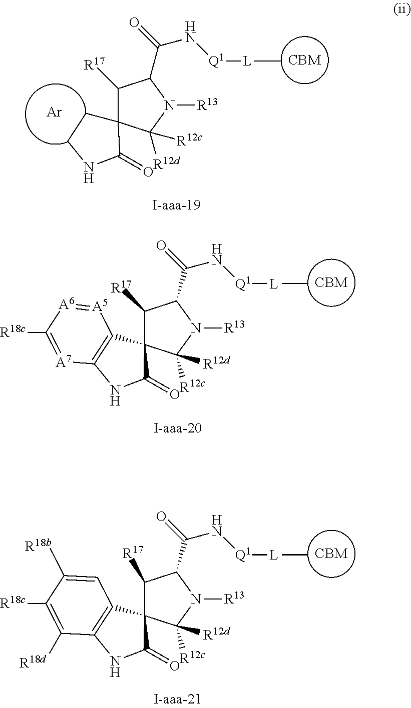

or a pharmaceutically acceptable salt thereof, wherein each of the variables R^{12c}, R^{12d}, R¹³, R¹⁷, R^{18b}, R^{18c}, R^{18d}, A⁵, A⁶, A⁷, Q¹, and Ar is as defined and described in WO 2017/176957 and US2019/127387.

Embodiment 14. The compound according to embodiment 3 or embodiment 13, wherein the MDM2 E3 ubiquitin ligase binding moiety is selected from

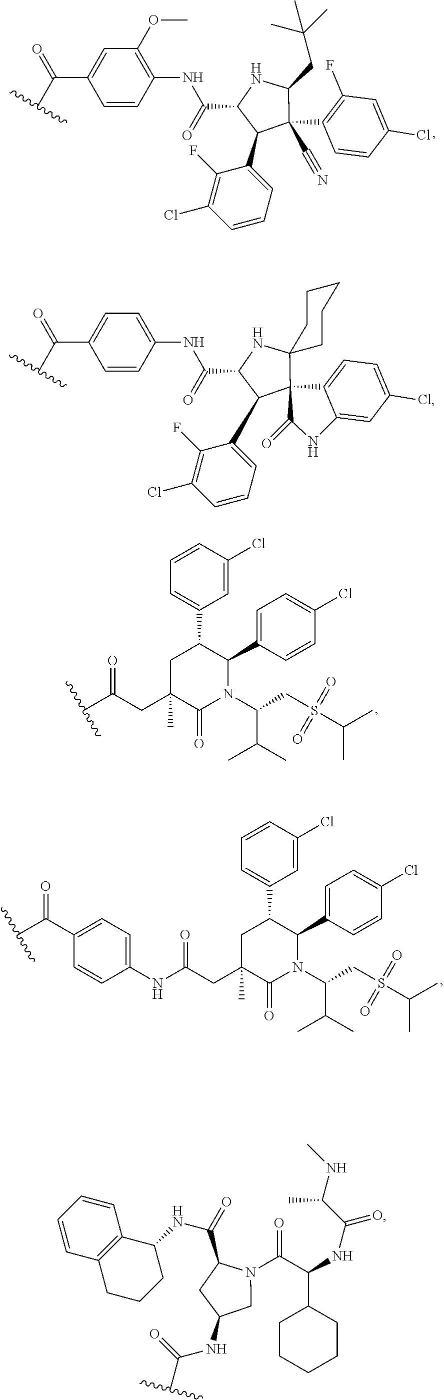

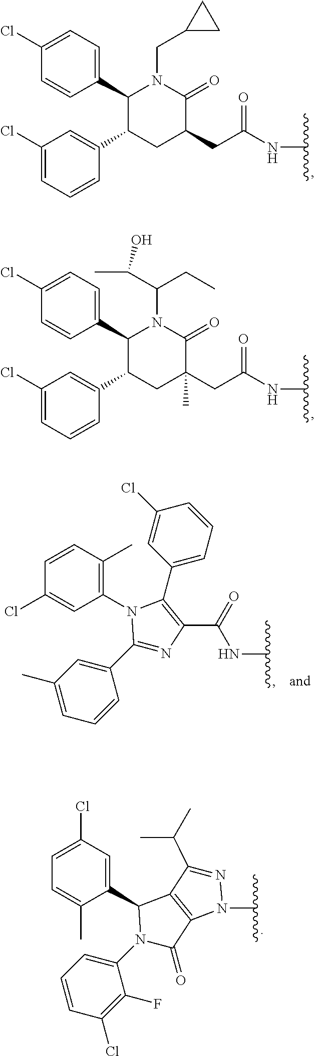 [SMILES: *NC(=O)C[C@H]1C[C@H](c2cccc(Cl)c2)[C@@H](c2ccc(Cl)cc2)N(CC2CC2)C1=O ; *NC(=O)C[C@@]1(C)C[C@H](c2cccc(Cl)c2)[C@@H](c2ccc(Cl)cc2)N(C(CC)[C@H](C)O)C1=O ; *NC(=O)c1nc(-c2cccc(C)c2)n(-c2cc(Cl)ccc2C)c1-c1cccc(Cl)c1 ; *n1nc(C(C)C)c2c1C(=O)N(c1cccc(Cl)c1F)[C@H]2c1cc(Cl)ccc1C]

Embodiment 15. The compound of any one of embodiments 1-14, wherein L is a covalent bond or a bivalent, saturated or partially unsaturated, straight or branched C₁₋₂₀ hydrocarbon chain, wherein 0-6 methylene units of L are independently replaced by -Cy-, —O—, —NR—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—.

Embodiment 16. The compound of any one of embodiments 1-15, wherein said compound is selected from any one of the compounds depicted in Table 1, or a pharmaceutically acceptable salt thereof.

Embodiment 17. A pharmaceutical composition comprising a compound of any one of embodiments 1-16, and a pharmaceutically acceptable carrier, adjuvant, or vehicle.

Embodiment 18. The pharmaceutical composition according to embodiment 17, further comprising an additional therapeutic agent.

Embodiment 19. A method of inhibiting or degrading CDK2 or CDK2 and CCNE1 in a patient or biological sample comprising administering to said patient, or contacting said biological sample with a compound according to any one of embodiments 1-16, or a pharmaceutical composition thereof.

Embodiment 20. A method of treating an CDK2-mediated disorder, disease, or condition in a patient comprising administering to said patient a compound according to any of one embodiments 1-16, or a pharmaceutical composition thereof.

Embodiment 21. The method of embodiment 20, wherein CDK2-mediated disorder, disease, or condition is cancer.

Embodiment 22. The method of embodiment 21, wherein the cancer the cancer is characterized by amplification or overexpression of CCNE1.

### EXEMPLIFICATION

### Abbreviations

Ac: acetyl
AcOH: acetic acid
ACN: acetonitrile
Ad: adamantly
AIBN: 2,2′-azo bisisobutyronitrile
Anhyd: anhydrous
Aq: aqueous
B₂Pin₂: bis (pinacolato)diboron -4,4,4′,4′,5,5,5′,5′-octamethyl-2,2′-bi(1,3,2-dioxaborolane)
BINAP: 2,2′-bis(diphenylphosphino)-1,1′-binaphthyl
BH₃: Borane
Bn: benzyl
Boc: tert-butoxycarbonyl
Boc₂O: di-tert-butyl dicarbonate
BPO: benzoyl peroxide
ⁿBuOH: n-butanol
CDI: carbonyldiimidazole
COD: cyclooctadiene
d: days
DABCO: 1,4-diazobicyclo[2.2.2]octane
DAST: diethylaminosulfur trifluoride
dba: dibenzylideneacetone
DBU: 1,8-diazobicyclo[5.4.0]undec-7-ene
DCE: 1,2-dichloroethane
DCM: dichloromethane
DEA: diethylamine
DHP: dihydropyran
DIBAL-H: diisobutylaluminum hydride
DIPA: diisopropylamine
DIPEA or DIEA: N,N-diisopropylethylamine
DMA: N,N-dimethylacetamide
DME: 1,2-dimethoxyethane
DMAP: 4-dimethylaminopyridine
DMF: N,N-dimethylformamide
DMP: Dess-Martin periodinane
DMSO-dimethyl sulfoxide
DPPA: diphenylphosphoryl azide
dppf: 1,1′-bis(diphenylphosphino)ferrocene
EDC or EDCI: 1-(3-dimethylaminopropyl)-3-ethylcarbodiimide hydrochloride
ee: enantiomeric excess
ESI: electrospray ionization
EA: ethyl acetate
EtOAc: ethyl acetate
EtOH: ethanol
FA: formic acid
h or hrs: hours
HATU: N,N,N′,N′-tetramethyl-O-(7-azabenzotriazol-1-yl)uronium hexafluorophosphate
HCl: hydrochloric acid
HPLC: high performance liquid chromatography
HOAc: acetic acid
IBX: 2-iodoxybenzoic acid
IPA: isopropyl alcohol
KHMDS: potassium hexamethyldisilazide
K₂CO₃: potassium carbonate
LAH: lithium aluminum hydride
LDA: lithium diisopropylamide
m-CPBA: meta-chloroperbenzoic acid
M: molar
MeCN: acetonitrile
MeOH: methanol
Me₂S: dimethyl sulfide
MeONa: sodium methylate
MeI: iodomethane
min: minutes
mL: milliliters
mM: millimolar
mmol: millimoles
MPa: mega pascal
MOMCl: methyl chloromethyl ether
MsCl: methanesulfonyl chloride
MTBE: methyl tert-butyl ether
nBuLi: n-butyllithium
NaNO₂: sodium nitrite
NaOH: sodium hydroxide
Na₂SO₄: sodium sulfate
NBS: N-bromosuccinimide
NCS: N-chlorosuccinimide
NFSI: N-Fluorobenzenesulfonimide
NMO: N-methylmorpholine N-oxide
NMP: N-methylpyrrolidine
NMR: Nuclear Magnetic Resonance
° C.: degrees Celsius
Pd/C: Palladium on Carbon
Pd(OAc)₂: Palladium Acetate
PBS: phosphate buffered saline
PE: petroleum ether
POCl₃: phosphorus oxychloride
PPh₃: triphenylphosphine
PyBOP: (Benzotriazol-1-yloxy)tripyrrolidinophosphonium hexafluorophosphate
Rel: relative
R.T. or rt: room temperature
sat: saturated
SEMCl: chloromethyl-2-trimethylsilylethyl ether
SFC: supercritical fluid chromatography
SOCl₂: sulfur dichloride
tBuOK: potassium tert-butoxide
TBAB: tetrabutylammonium bromide
TBAI: tetrabutylammonium iodide
TEA: triethylamine
Tf: trifluoromethanesulfonate
TfAA, TFMSA or Tf₂O: trifluoromethanesulfonic anhydride
TFA: trifluoracetic acid
TIPS: triisopropylsilyl
THF: tetrahydrofuran
THP: tetrahydropyran
TLC: thin layer chromatography
TMEDA: tetramethylethylenediamine
pTSA: para-toluenesulfonic acid
wt: weight
Xantphos: 4,5-bis(diphenylphosphino)-9,9-dimethylxanthene

General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

Analytical instruments Table:

| | |
|---|---|
| LCMS | Shimadzu UFLC MS: LCMS-2020 |
| | Agilent Technologies 1200 series MS: Agilent |
| | Technologies 6110 |
| | Agilent Technologies 1200 series MS: LC/MSD VL |
| NMR | BRUKER AVANCE III/400; Frequency (MHz) |
| | 400.13; Nucleus: 1H; Number of Transients: 8 |
| Prep-HPLC | Gilson GX-281 systems: instruments GX-A, GX-B, |
| | GX-C, GX-D, GX-E, GX-F, GX-G and GX-H |
| GCMS | SHIMADZU GCMS-QP2010 Ultra |
| Analytical cSFC | Agilent Technologies 1290 Infinity |
| Prep-cSFC | Waters SFC Prep 80 |

For acidic LCMS data:

LCMS was recorded on an Agilent 1200 Series LC/MSD or Shimadzu LCMS2020 equipped with electro-spray ionization and quadruple MS detector [ES+ve to give MH+] and equipped with Chromolith Flash RP-18e 25*2.0 mm, eluting with 0.0375 vol % TFA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A) 2 mM Ammonium Acetate in 0.1% Formic Acid in Water and (B) 0.1% Formic Acid in Acetonitrile.

For basic LCMS data:

LCMS was recorded on an Agilent 1200 Series LC/MSD or Shimadzu LCMS 2020 equipped with electro-spray ionization and quadruple MS detector [ES+ve to give MH+] and equipped with Xbridge C18, 2.1×50 mm columns packed with 5 mm C18-coated silica or Kinetex EVO C18 2.1×30 mm columns packed with 5 mm C18-coated silica, eluting with 0.05 vol % NH₃H₂O in water (solvent A) and acetonitrile (solvent B).

HPLC Analytical Method:

HPLC was carried out on X Bridge C18 150*4.6 mm, 5 micron. Column flow was 1.0 ml/min and mobile phase were used (A) 0.1% Ammonia in water and (B) 0.1% Ammonia in Acetonitrile.

Prep HPLC Analytical Method:

The compound was purified on Shimadzu LC-20AP and UV detector. The column used was X-BRIDGE C18 (250*19)mm, 5μ. Column flow was 16.0 ml/min. Mobile phase were used (A) 0.1% Formic Acid in Water and (B) Acetonitrile Basic method used (A) 5 mM ammonium bicarbonate and 0.1% NH3 in Water and (B) Acetonitrile or (A) 0.1% Ammonium Hydroxide in Water and (B) Acetonitrile. The UV spectra were recorded at 202 nm & 254 nm.

NMR Method:

The ¹H NMR spectra were recorded on a Bruker Ultra Shield Advance 400 MHz/5 mm Probe (BBFO). The chemical shifts are reported in part-per-million.

### Example 1. Synthesis of Intermediates

### Synthesis of 4-[[tert-butyl(diphenyl)silyl]oxymethyl]cyclohexanol (Intermediate CT)

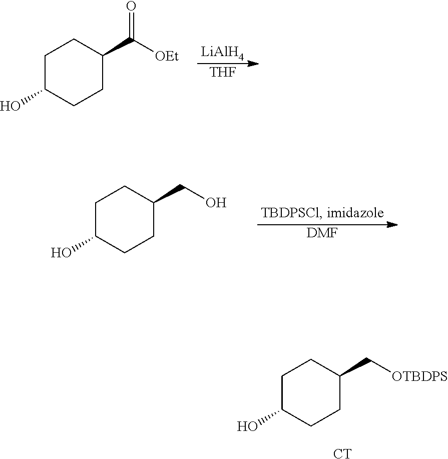

### Step 1—4-(Hydroxymethyl)cyclohexanol

To a solution of LiAlH₄ (3.31 g, 87.1 mmol) in THF (30 mL), was added ethyl 4-hydroxycyclohexanecarboxylate (10.0 g, 58.0 mmol, CAS #3618-04-0) in THF (100 mL) dropwise at 0° C., then the mixture was stirred at 0° C. for 5 hrs. On completion, the mixture was quenched with H₂O (3.3 mL), then a solution of 15% NaOH (3.3 mL) was added dropwise. The mixture was dried with anhydrous Na₂SO₄, filtered and the filtrate was concentrated in vacuo to give the title compound (7.5 g, 99% yield) as yellow solid. ¹H NMR (400 MHz, DMSO-d₆) δ 3.37-3.23 (m, 1H), 3.17 (d, J=6.0 Hz, 2H), 1.85-1.75 (m, 2H), 1.75-1.62 (m, 2H), 1.30-1.16 (m, 1H), 1.14-0.95 (m, 2H), 0.93-0.72 (m, 2H).

### Step 2-4-[[Tert-butyl(diphenyl)silyl]oxymethyl]cyclohexanol

To a solution of 4-(hydroxymethyl)cyclohexanol (6.5 g, 49.9 mmol) and imidazole (4.08 g, 59.9 mmol) in DMF (200 mL) was added TBDPSCl (14.4 g, 52.4 mmol) at 0° C. The mixture was stirred at 25° C. for 12 hrs. On completion, the mixture was diluted with H₂O (100 mL), and extracted with EA (2×30 mL). The organic layers were washed with brine (2×30 mL), dried over anhydrous Na₂SO₄, filtered and concentrated in vacuo. The mixture was purified by silica gel column (PE:EA=5:1) to give the title compound (9.10 g, 49% yield) as yellow oil. ¹H NMR (400 MHz, CDCl₃) δ 7.70-7.60 (m, 4H), 7.48-7.31 (m, 6H), 3.63-3.51 (m, 1H), 3.47 (d, J=6.0 Hz, 2H), 2.05-1.95 (m, 2H), 1.89-1.80 (m, 2H), 1.50-1.45 (m, 1H), 1.31-1.22 (m, 2H), 1.10-1.00 (m, 2H), 1.05 (s, 9H).

### Synthesis of (4-Allyloxycyclohexyl)methoxy-tert-butyl-diphenyl-silane (Intermediate CU)

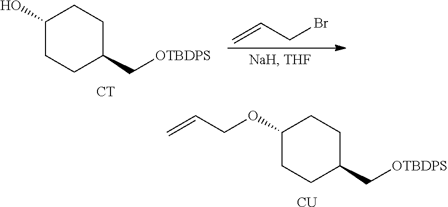

To a solution of 4-[[tert-butyl(diphenyl)silyl]oxymethyl]cyclohexanol (500 mg, 1.36 mmol, Intermediate CT) in THF (5 mL) was added NaH (81.3 mg, 2.03 mmol, 60% dispersion in mineral oil) at 0° C. After addition, the mixture was stirred at this temperature for 30 minutes, then 3-bromoprop-1-ene (656 mg, 5.43 mmol, 0.3 mL, CAS #106-95-6) was added dropwise. The mixture was stirred at 25° C. for 4 hrs. On completion, the mixture was quenched with H₂O (1 mL) at 25° C., diluted with H₂O (10 mL) and extracted with EA (3×10 mL). The combined organic layers were washed with brine (2×5 mL), dried over anhydrous Na₂SO₄, filtered and concentrated in vacuo to give a residue. The residue was purified by prep-TLC (SiO₂, DCM:MeOH=10:1) to give the title compound (210 mg, 37% yield) as a white solid. ¹H NMR (400 MHz, CDCl₃) δ 7.59-7.55 (m, 4H), 7.33-7.26 (m, 6H), 5.91-5.78 (m, 1H), 5.22-5.13 (m, 1H), 5.10-5.02 (m, 1H), 3.98-3.89 (m, 2H), 3.37 (d, J=6.4 Hz, 2H), 3.18-3.08 (m, 1H), 2.01-1.95 (m, 2H), 1.79-1.73 (m, 2H), 1.45-1.41 (m, 1H), 0.96 (s, 9H), 0.93-0.88 (m, 2H), 0.80-0.76 (m, 2H).

### Synthesis of N2-(4-benzylsulfanyl-2-methyl-phenyl)-5-bromo-N4-cyclopentyl-pyrimidine-2,4-diamine (Intermediate CV)

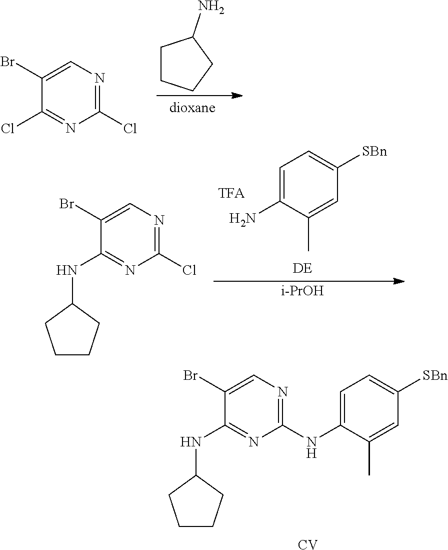

### Step 1—5-bromo-2-chloro-N-cyclopentylpyrimidin-4-amine

To a solution of 5-bromo-2,4-dichloro-pyrimidine (10 g, 43.8 mmol, CAS #36082-50-5) in dioxane (100 mL) was added cyclopentanamine (4.48 g, 52.6 mmol, CAS #1003-03-8) at 0° C. under nitrogen flow. Then the reaction was stirred at 20° C. for 6 h under nitrogen atmosphere. On completion, the reaction was poured into ice water (100 mL) then extracted with ethyl acetate (150 mL×2). The combined organic phase is washed with brine (70 mL×2), dried over sodium sulfate, then filtered and the filtrate was concentrated to give a residue. The residue was purified by column chromatography on silica gel (eluted with petroleum ether: ethyl acetate=100:1 to 100:15) to give the title compound (4.7 g, 38% yield) as a yellow solid. ¹H NMR (400 MHz, CDCl₃) δ=8.10 (s, 1H), 5.45 (br s, 1H), 4.57-4.29 (m, 1H), 2.20-2.08 (m, 2H), 1.82-1.57 (m, 4H), 1.48 (qd, J=6.4, 12.8 Hz, 2H).

### Step 2—N2-(4-(benzylthio)-2-methylphenyl)-5-bromo-N4-cyclopentylpyrimidine-2,4-diamine

To a solution of 5-bromo-2-chloro-N-cyclopentyl-pyrimidin-4-amine (2.65 g, 9.59 mmol) in isopropanol (40 mL) was added 4-benzylsulfanyl-2-methyl-aniline (2 g, 8.72 mmol, Intermediate DE) and TFA (19.8 g, 174 mmol) at 20° C. under nitrogen flow. Then the reaction was stirred at 80° C. for 10 h under nitrogen atmosphere. On completion, the reaction was poured into ice water (40 mL) and extracted with EtOAc (50 mL×2). The combined organic phase is washed with brine (30 mL×2), dried over sodium sulfate, then the mixture was filtered and concentrated to give a residue. The residue was purified by column chromatography on silica gel (eluted with petroleum ether: ethyl acetate=100:1 to 100:15) to give the title compound (3.4 g, 83.0% yield) as a white solid. ¹H NMR (400 MHz, CDCl₃) δ=11.05 (s, 1H), 7.80 (s, 1H), 7.35-7.23 (m, 6H), 7.20 (d, J=1.8 Hz, 1H), 7.13 (dd, J=2.0, 8.3 Hz, 1H), 5.99 (br d, J=6.8 Hz, 1H), 4.18-4.06 (m, 3H), 2.28 (s, 3H), 2.00-1.85 (m, 2H), 1.79-1.56 (m, 4H), 1.52-1.44 (m, 2H).

### Synthesis of 4-[(6-Chloro-8-cyclopentyl-7-oxo-pyrido[2,3-d]pyrimidin-2-yl)amino]-3-methyl-benzenesulfonyl chloride (Intermediate CW)

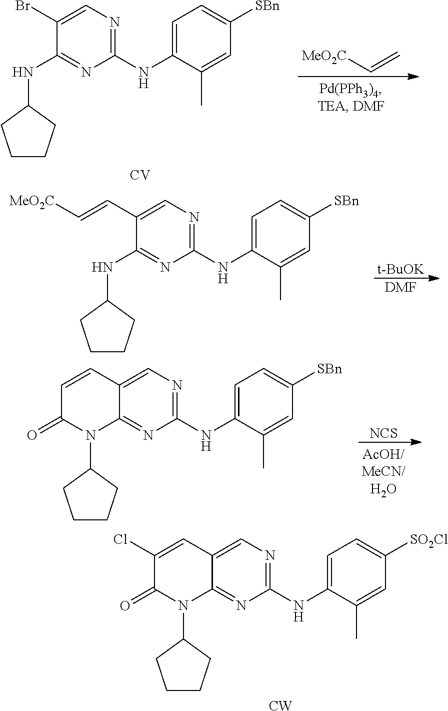

### Step 1—Methyl (E)-3-[2-(4-benzylsulfanyl-2-methyl-anilino)-4-(cyclopentylamino) pyrimidin-5-yl]prop-2-enoate

A mixture of N2-(4-benzylsulfanyl-2-methyl-phenyl)-5-bromo-N4-cyclopentyl-pyrimidine-2,4-diamine (10 g, 21.3 mmol, Intermediate CV), methyl prop-2-enoate (12.6 g, 146 mmol, CAS #96-33-3), TEA (6.47 g, 63.9 mmol), and Pd(PPh₃)₄ (2.46 g, 2.13 mmol) in DMF (200 mL) was degassed and purged with N₂ three times. Then the mixture was stirred at 90° C. for 36 hours under N₂ atmosphere. On completion, the reaction mixture was quenched with H₂O (100 mL) at 25° C., and extracted with EA (3×200 mL). The combined organic layers were washed with brine (2×100 mL), dried over with anhydrous Na₂SO₄, filtered and concentrated in vacuo to give a residue. The residue was purified by prep-HPLC (FA condition) to afford the title compound (15.6 g, 70% yield) ¹H NMR (400 MHz, DMSO-d₆) δ 8.50 (s, 1H), 8.32 (s, 1H), 7.82 (d, J=15.6 Hz, 1H), 7.56 (d, J=8.4 Hz, 1H), 7.34-7.30 (m, 3H), 7.29-7.25 (m, 3H), 7.18 (s, 1H), 7.11 (dd, J=1.6, 8.4 Hz, 1H), 6.31 (d, J=15.6 Hz, 1H), 4.28-4.23 (m, 1H), 4.18 (s, 2H), 3.69 (s, 3H), 2.19 (s, 3H), 1.88-1.82 (m, 2H), 1.70-1.66 (m, 2H), 1.52-1.46 (m, 4H). LC-MS (ESI⁺) m z 475.2 (M+H)⁺.

### Step 2—2-(4-Benzylsulfanyl-2-methyl-anilino)-8-cyclopentyl-pyrido[2,3-d]pyrimidin-7-one

To a solution of methyl (E)-3-[2-(4-benzylsulfanyl-2-methyl-anilino)-4-(cyclopentylamino) pyrimidin-5-yl]prop-2-enoate (7.8 g, 16.4 mmol) in DMF (80 mL) was added t-BuOK (5.53 g, 49.3 mmol). The mixture was stirred at 25° C. for 30 min. Then the mixture was heated to 120° C. and stirred for 1 hr. On completion, the reaction mixture was quenched with H₂O (200 mL) and extracted with EA (2×300 mL). The combined organic layers were washed with brine (2×100 mL), dried over with anhydrous Na₂SO₄, filtered and concentrated in vacuo. The residue was purified by column chromatography (SiO₂, Petroleum ether/Ethyl acetate=40/1 to 10/1, Rf=0.24) to give the title compound (4.60 g, 63% yield) as a yellow solid. LC-MS (ESI⁺) m/z 443.0 (M+H)⁺.

### Step 3—4-[(6-Chloro-8-cyclopentyl-7-oxo-pyrido [2, 3-d]pyrimidin-2-yl) amino]-3-methyl-benzenesulfonyl chloride

To a solution of 2-(4-benzylsulfanyl-2-methyl-anilino)-8-cyclopentyl-pyrido [2,3-d]pyrimidin-7-one (2 g, 4.52 mmol) in ACN (20 mL), AcOH (2 mL), and H₂O (0.5 mL) was added NCS (2.41 g, 18 mmol) in the dark. The mixture was stirred at 25° C. for 0.5 hr in the dark. The reaction mixture was diluted with H₂O (50 mL) and extracted with EA (3×50 mL). The combined organic layers were washed with brine (2×60 mL), dried over with anhydrous Na₂SO₄, filtered and concentrated in vacuo to give a residue. The residue was purified by column chromatography (SiO₂, Petroleum ether/Ethyl acetate=40/1 to 10/1, Rf=0.40) to afford the title compound (1.49 g, 72% yield) as a yellow solid. ¹H NMR (400 MHz, DMSO-d₆) δ 9.61 (s, 1H), 8.71 (s, 1H), 8.15 (s, 1H), 7.50 (s, 1H), 7.45-7.35 (m, 2H), 5.71 (s, 1H), 2.22 (s, 3H), 2.15-2.04 (m, 2H), 1.69 (s, 4H), 1.44 (s, 2H).

### Synthesis of 4-[3-[4-[(6-Chloro-8-cyclopentyl-7-oxo-pyrido[2,3-d]pyrimidin-2-yl)amino]-3-methyl-phenyl]sulfonylpropoxy]cyclohexanecarbaldehyde (Intermediate CX)

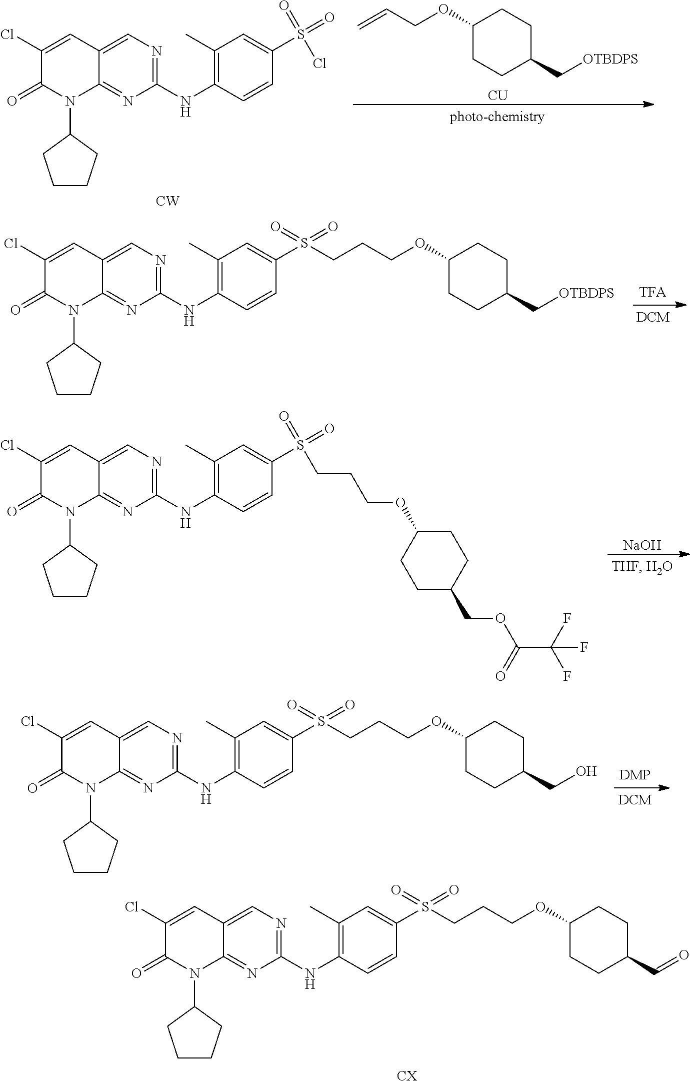

### Step 1—2-[4-[3-[4-[[Tert-butyl(diphenyl)silyl]oxymethyl]cyclohexoxy]propylsulfonyl]-2-methyl-anilino]-6-chloro-8-cyclopentyl-pyrido [2,3-d]pyrimidin-7-one

To a solution of (4-allyloxycyclohexyl)methoxy-tert-butyl-diphenyl-silane (50.0 mg, 122 umol, Intermediate CU) in ACN (3 mL) was added IR(PPY)₃ (400 ug), 4-mercaptophenol (3.09 mg, 24.4 umol) over 30 minutes. Then 4-[(6-chloro-8-cyclopentyl-7-oxo-pyrido[2,3-d]pyrimidin-2-yl) amino]-3-methyl-benzenesulfonyl chloride (138 mg, 305 umol, Intermediate CW) was added dropwise. The mixture was stirred at 25° C. for 12 hrs. On completion, the mixture was filtered and concentrated in vacuo to give a residue. The residue was purified by prep-TLC (SiO₂, PE:EA=0:1) to give the title compound (50 mg, 49% yield) as a white solid. ¹H NMR (400 MHz, CDCl₃) δ 8.48 (s, 1H), 8.35 (d, J=8.4 Hz, 1H), 7.71-7.64 (m, 2H), 7.54 (d, J=6.8 Hz, 4H), 7.27 (s, 5H), 7.19-7.14 (m, 2H), 5.89-5.78 (m, 1H), 3.47-3.39 (m, 2H), 3.34 (d, J=6.0 Hz, 2H), 3.16-3.08 (m, 2H), 3.05-2.94 (m, 1H), 2.36 (s, 3H), 2.19 (d, J=7.2 Hz, 2H), 1.98 (s, 2H), 1.91-1.78 (m, 6H), 1.71 (d, J=12.0 Hz, 2H), 1.59 (d, J=2.8 Hz, 2H), 1.37 (s, 1H), 1.26-1.13 (m, 1H), 1.10-0.99 (m, 2H), 0.94 (s, 9H), 0.86 (d, J=12.4 Hz, 2H). LC-MS (ESI⁺) m/z 827.5 (M+H)⁺.

### Step 2—[4-[3-[4-[(6-Chloro-8-cyclopentyl-7-oxo-pyrido[2,3-d]pyrimidin-2-yl)amino]-3-methyl-phenyl]sulfonylpropoxy]cyclohexyl]methyl 2,2,2-trifluoroacetate

To a solution of 2-[4-[3-[4-[[tert-butyl(diphenyl)silyl]oxymethyl]cyclohexoxy]propylsulfonyl]-2-methyl-anilino]-6-chloro-8-cyclopentyl-pyrido[2,3-d]pyrimidin-7-one (50.0 mg, 60.4 umol) in DCM (0.5 mL) was added TFA (770 mg, 6.75 mmol, 0.5 mL). The mixture was stirred at 25° C. for 2 hrs. On completion, the mixture was filtered and concentrated in vacuo to give a residue. The residue was purified by prep-TLC (SiO₂, PE:EA=0:1) to give the title compound (32.0 mg, 75% yield) as a yellow solid. LC-MS (ESI⁺) m/z 684.9 (M+H)⁺.

### Step 3—6-Chloro-8-cyclopentyl-2-[4-[3-[4-(hydroxymethyl)cyclohexoxy]propylsulfonyl]-2-methyl-anilino]pyrido [2,3-d]pyrimidin-7-one

To a solution of [4-[3-[4-[(6-chloro-8-cyclopentyl-7-oxo-pyrido[2,3-d]pyrimidin-2-yl) amino]-3-methyl-phenyl]sulfonylpropoxy]cyclohexyl]methyl 2,2,2-trifluoroacetate (32.0 mg, 46.7 umol) in THF (1 mL) was added NaOH (0.373 mg, 9.34 umol) in H₂O (1 mL). The mixture was stirred at 25° C. for 20 min. On completion, the mixture was concentrated in vacuo to give the title (27.0 mg, 92% yield) as a white solid. LC-MS (ESI⁺) m/z 589.2 (M+H)⁺.

### Step 4—4-[3-[4-[(6-Chloro-8-cyclopentyl-7-oxo-pyrido[2,3-d]pyrimidin-2-yl)amino]-3-methyl-phenyl]sulfonylpropoxy]cyclohexanecarbaldehyde

To a solution of 6-chloro-8-cyclopentyl-2-[4-[3-[4-(hydroxymethyl)cyclohexoxy]propylsulfonyl]-2-methyl-anilino]pyrido[2,3-d]pyrimidin-7-one (27.0 mg, 45.8 umol) in DCM (1 mL) was added DMP (38.8 mg, 91.6 umol, 28.5 uL). The mixture was stirred at 25° C. for 3 hrs. On completion, the reaction mixture was quenched with Na₂S₂O₃ (0.5 mL) at 25° C., and then diluted with NaHCO₃ (8 mL) and extracted with DCM (3×8 mL). The combined organic layers were washed with brine (2×5 mL), dried over anhydrous Na₂SO₄, filtered and concentrated in vacuo to give the title compound (31.0 mg, 98% yield) as yellow oil. LC-MS (ESI⁺) m/z 587.2 (M+H)⁺.

### Synthesis of [1-[(4-Methoxyphenyl) methyl]-2,6-dioxo-3-piperidyl]trifluoromethanesulfonate (Intermediate CY)

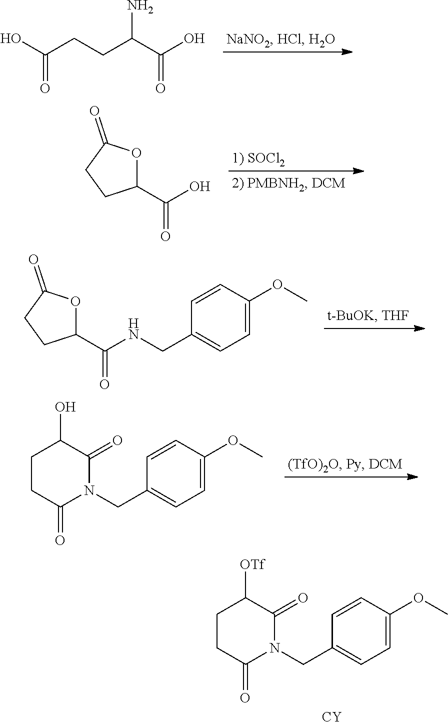 [SMILES: NC(CCC(=O)O)C(=O)O ; O=C1CCC(C(=O)O)O1 ; COc1ccc(CNC(=O)C2CCC(=O)O2)cc1 ; COc1ccc(CN2C(=O)CCC(O)C2=O)cc1 ; COc1ccc(CN2C(=O)CCC(OS(=O)(=O)C(F)(F)F)C2=O)cc1 ; "CY"]

### Step 1—5-Oxotetrahydrofuran-2-carboxylic acid

To a solution of 2-aminopentanedioic acid (210 g, 1.43 mol, CAS #617-65-2) in H₂O (800 mL) and HCl (12 M, 210 mL) was added a solution of NaNO₂ (147 g, 2.13 mol) in H₂O (400 mL) at -5° C. The mixture was stirred at 15° C. for 12 hrs. On completion, the mixture was concentrated and then dissolved in EA (500 mL) and filtered and washed with EA (3×100 mL). The filtrate and washed solution were dried over Na₂SO₄, filtered and concentrated in vacuo to give the title compound (200 g, crude) as yellow oil. ¹H NMR (400 MHz, CDCl₃) δ 6.43 (s, 1H), 5.02-4.95 (m, 1H), 2.67-2.38 (m, 4H)

### Step 2—N-[(4-methoxyphenyl)methyl]-5-oxo-tetrahydrofuran-2-carboxamide

To 5-oxotetrahydrofuran-2-carboxylic acid (120 g, 922 mmol) was added SOCl₂ (246 g, 2.07 mol) at 0° C. slowly. The mixture was stirred at 85° C. for 3 hrs, and then the mixture was stirred at 15° C. for 6 hrs. The mixture was concentrated in vacuo. The residue was dissolved in dry DCM (1 L) at 0° C. under N₂. After that a solution of Et₃N (187 g, 1.84 mol) and 4-methoxybenzylamine (101 g, 738 mmol) in DCM (400 mL) was added, then the mixture was stirred at 15° C. for 3 hrs. On completion, water (600 mL) was added and the mixture was extracted with DCM (3×300 mL). The combined organic phase was washed with 0.5 M HCl (500 mL), brine (500 mL), dried over with anhydrous sodium sulfate and filtered. The filtrate was concentrated in vacuo and the residue was purified by flash silica gel chromatography (PE:EA=1:1) to give the title compound (138 g, 60% yield) as a yellow solid. ¹H NMR (400 MHz, CDCl₃) δ 7.22-7.20 (d, J=8.0, 1H), 6.89-6.87 (d, J=8.0, 1H), 4.90-4.86 (m, 1H), 4.47-4.4.36 (m, 2H) 3.81 (s, 3H), 2.67-2.64 (m, 1H), 2.59-2.54 (m, 2H), 2.40-2.38 (m, 1H); LC-MS (ESI⁺) m/z 272.0 (M+Na)⁺.

### Step 3—3-Hydroxy-1-[(4-methoxyphenyl)methyl]piperidine-2,6-dione

A solution of N-[(4-methoxyphenyl)methyl]-5-oxo-tetrahydrofuran-2-carboxamide (138 g, 553 mmol) in anhydrous THF (1500 mL) was cooled to −78° C. Then, t-BuOK (62.7 g, 559 mmol) in a solution of anhydrous THF (1000 mL) was added dropwise slowly at −78° C. under nitrogen atmosphere. The resulting reaction mixture was stirred at −40° C. for 1 hr. On completion, the reaction mixture was quenched with saturated NH₄Cl solution (100 mL). The mixture was extracted with ethyl acetate (3×1500 mL). The combined organic layer was washed with brine (300 mL), dried over anhydrous sodium sulfate, filtered and the filtrate was concentrated in vacuo. The residue was purified by silica gel chromatography (PE:EA=1:1) to give the title compound (128 g, 92% yield) as a white solid. ¹H NMR (400 MHz, CDCl₃) δ 7.39-7.32 (m, 2H), 6.89-6.81 (m, 2H), 4.91 (s, 2H), 4.17-4.11 (m, 1H), 3.80 (s, 3H), 3.54 (s, 1H), 2.98-2.87 (m, 1H), 2.73-2.60 (m, 1H), 2.26-2.20 (m, 1H), 1.80 (dq, J=4.8, 13.1 Hz, 1H).

### Step 4—[1-[(4-Methoxyphenyl) methyl]-2,6-dioxo-3-piperidyl] trifluoromethanesulfonate

To a solution of 3-hydroxy-1-[(4-methoxyphenyl) methyl] piperidine-2, 6-dione (43.0 g, 173 mmol) and pyridine (27.3 g, 345 mmol) in DCM (500 mL) was added trifluoromethylsulfonyl trifluoromethanesulfonate (73.0 g, 258 mmol) dropwise at 0° C. The mixture was stirred at −10° C. for 1.5 hours under N₂. On completion, the mixture was concentrated in vacuo. The residue was purified by column chromatography on silica gel (PE:EA=20:1/8:1) to give the title compound (45.0 g, 68% yield) as light yellow gum. ¹H NMR (400 MHz, CDCl₃) δ 7.36 (d, J=8.4 Hz, 2H), 6.85-6.82 (m, 2H), 5.32-5.28 (m, 1H), 4.91 (s, 2H), 3.79 (s, 3H), 3.02-2.97 (m, 1H), 2.79-2.74 (m, 1H), 2.41-2.35 (m, 2H).

### Synthesis of 5-Bromo-3-methyl-1H-benzimidazol-2-one (Intermediate CZ)

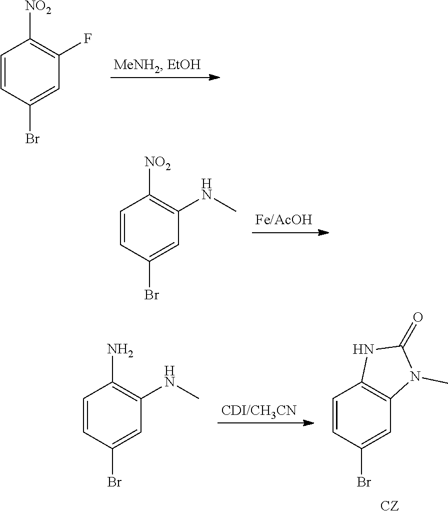

### Step 1—5-Bromo-N-methyl-2-nitro-aniline

4-bromo-2-fluoro-1-nitro-benzene (230 g, 1.05 mol, CAS #321-23-3) was added to a solution of mehylamine in tetrahydrofuran (2 M, 1.51 L). The mixture was stirred at 15° C. for 10 minutes. On completion, the mixture was diluted with H₂O (250 mL) and extracted with EtOAc (3×300 mL). The combined organic layers were washed with brine (300 mL), dried over Na₂SO₄, filtered and concentrated in vacuo to give the title compound (200 g, 83% yield) as a yellow solid. ¹H NMR (400 MHz, DMSO-d₆) δ 8.22 (s, 1H), 7.98 (d, J=9.2 Hz, 1H), 7.16 (d, J=1.6 Hz, 1H), 6.82 (dd, J=8.4, 1.6 Hz, 1H), 2.95 (d, J=4.8 Hz, 3H).

### Step 2—4-Bromo-N2-methyl-benzene-1,2-diamine

To a mixture of 5-bromo-N-methyl-2-nitro-aniline (200 g, 865 mmol) in EtOAc (1 L) and H₂O (500 mL) was added AcOH (1.00 L). The mixture was warmed to 50° C., and then Fe (174 g, 3.11 mol) was added to the reaction mixture. After that, the reaction mixture was stirred at 80° C. for 6 hours. On completion, the mixture was filtered through celite. The filtrate was concentrated in vacuo and the residue was diluted with H₂O (250 mL) and extracted with EtOAc (3×300 mL). The combined organic layers were washed with aq.NaHCO₃ and brine (300 mL), dried over Na₂SO₄, filtered and concentrated in vacuo. The residue was purified by flash silica gel chromatography to give the title compound (130 g, 75% yield) as black oil. ¹H NMR (400 MHz, DMSO-d₆) δ 6.55-6.52 (m, 1H), 6.48-6.45 (m, 1H), 6.43-6.42 (m, 1H), 4.89-4.88 (m, 1H), 4.61 (s, 2H), 2.70 (d, J=4.0 Hz, 3H).

### Step 3—5-Bromo-3-methyl-1H-benzimidazol-2-one

To a solution of 4-bromo-N2-methyl-benzene-1,2-diamine (110 g, 547 mmol) in CH₃CN (1.3 L) was added CDI (177 g, 1.09 mol). The mixture was stirred at 80° C. for 6 hours under N₂. On completion, the mixture was concentrated in vacuo. The mixture was diluted with H₂O (1.0 L) and filtered. The filter cake was washed with water (3×200 mL) and dried in vacuo to give the title compound (106 g, 85% yield) as a white solid. ¹H NMR (400 MHz, DMSO-d₆) δ 11.00 (s, 1H), 7.33 (s, 1H), 7.13 (d, J=8.0 Hz, 1H), 6.92 (d, J=8.0 Hz, 1H), 3.27 (s, 3H).

### Synthesis of 3-(5-bromo-3-methyl-2-oxo-benzimidazol-1-yl)piperidine-2,6-dione (Intermediate DA)

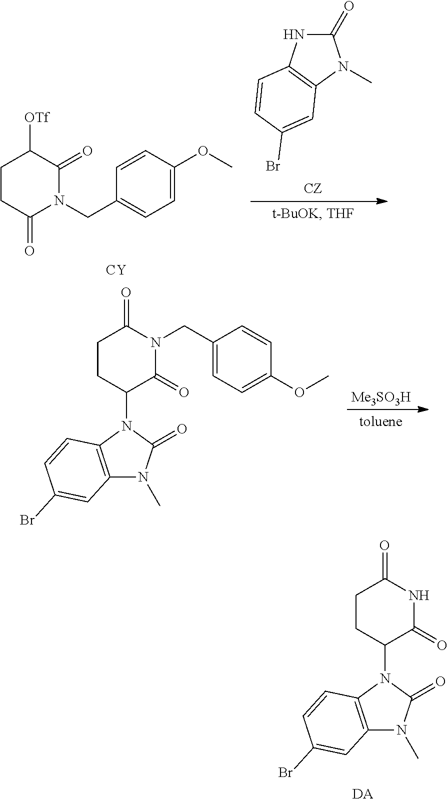

### Step 1—3-(5-Bromo-3-methyl-2-oxo-benzimidazol-1-yl)-1-[(4-methoxyphenyl)methyl]piperidine-2,6-dione

To a solution of 5-bromo-3-methyl-1H-benzimidazol-2-one (4.90 g, 21.6 mmol, Intermediate CZ) in THF (300 mL) was added t-BuOK (3.63 g, 32.3 mmol) at 0° C. The mixture was stirred at 0-10° C. for 1 hour under N₂. Then a solution of [1-[(4-methoxyphenyl) methyl]-2, 6-dioxo-3-piperidyl]trifluoromethanesulfonate (9.87 g, 25.9 mmol, Intermediate CY) in THF (100 mL) was added to the reaction mixture at 0-10° C. during 30 minutes. The mixture was stirred at 0-10° C. for 30 minutes under N₂. An additional solution of [1-[(4-methoxyphenyl) methyl]-2, 6-dioxo-3-piperidyl] trifluoromethanesulfonate (2.47 g, 6.47 mmol) in THF (20 mL) was added to the reaction mixture at 0-10° C. dropwise. The mixture was then stirred at 0-10° C. for another 30 minutes under N₂. On completion, the reaction was quenched water (400 mL) and extracted with EA (3×200 mL). The combined organic layer was concentrated in vacuo. The residue was triturated with EA (80 mL) and filtered. The filter cake was collected and dried in vacuo to give the title compound (6.70 g, 67% yield) as light yellow solid. The filtrate was also concentrated in vacuo and the residue was purified by column chromatography to give another batch title compound (1.80 g, 18% yield) as light yellow solid. ¹H NMR (400 MHz, DMSO-d₆) δ 7.47 (d, J=1.6 Hz, 1H), 7.21-7.16 (m, 3H), 7.01 (d, J=8.0 Hz, 1H), 6.85 (d, J=8.8 Hz, 2H), 5.55-5.51 (m, 1H), 4.84-4.73 (m, 2H), 3.72 (s, 3H), 3.33 (s, 3H), 3.04-3.00 (m, 1H), 2.83-2.67 (m, 2H), 2.07-2.05 (m, 1H).

### Step 2—3-(5-Bromo-3-methyl-2-oxo-benzimidazol-1-yl)piperidine-2,6-dione

To a mixture of 3-(5-bromo-3-methyl-2-oxo-benzimidazol-1-yl)-1-[(4-methoxyphenyl)methyl]piperidine-2,6-dione (8.50 g, 18.6 mmol) in toluene (50 mL) was added methanesulfonic acid (33.8 g, 351 mmol, 25 mL) at room temperature (15° C.). The mixture was stirred at 120° C. for 2 hours. On completion, the reaction mixture was cooled to room temperature and concentrated in vacuo. The residue was poured into ice/water (200 mL), and extracted with EA (3×100 mL). The combined organic layer was washed with brine (50 mL), dried over Na₂SO₄, filtered and concentrated in vacuo. The residue was triturated with EA (80 mL) and filtered. The filtrate cake was collected and dried in vacuo to give the title compound (4.20 g, 67% yield) as off-white solid. ¹H NMR (400 MHz, DMSO-d₆) δ 11.12 (s, 1H), 7.47 (d, J=2.0 Hz, 1H), 7.22 (d, J=8.4 Hz, 1H), 7.10 (d, J=8.4 Hz, 1H), 5.40-5.35 (m, 1H), 2.34 (s, 3H), 2.92-2.88 (m, 1H), 2.71-2.60 (m, 2H), 2.03-1.99 (m, 1H).

### Synthesis of 3-[3-Methyl-2-oxo-5-(4-piperidyl)benzimidazol-1-yl]piperidine-2,6-dione (Intermediate DB)

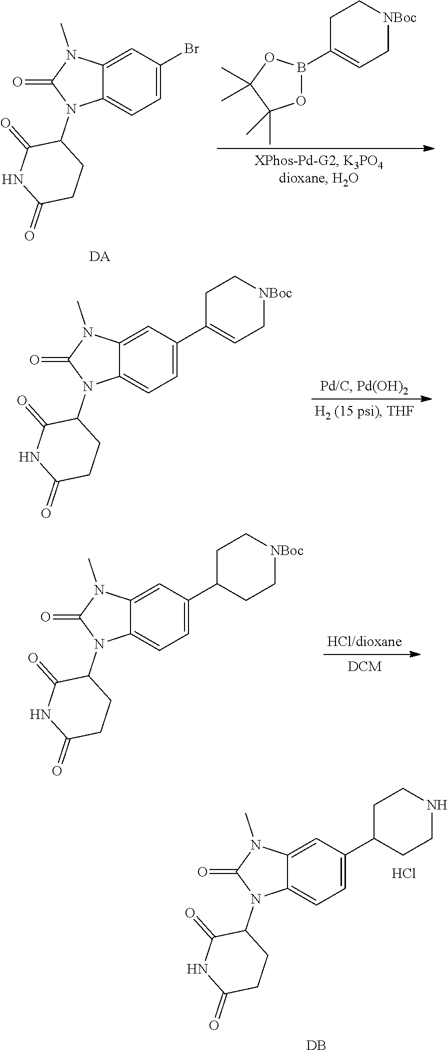

### Step 1—Tert-butyl 4-[1-(2,6-dioxo-3-piperidyl)-3-methyl-2-oxo-benzimidazol-4-yl]-3,6-dihydro-2H-pyridine-1-carboxylate

To a solution of 3-(5-bromo-3-methyl-2-oxo-benzimidazol-1-yl)piperidine-2,6-dione (5.00 g, 14.8 mmol, Intermediate DA), tert-butyl 4-(4,4,5,5-tetramethyl-1,3,2-dioxaborolan-2-yl)-3,6-dihydro-2H-pyridine-1-carboxylate (5.49 g, 17.7 mmol, CAS #286961-14-6), K₃PO₄ (6.28 g, 29.6 mmol) and [2-(2-aminophenyl)phenyl]-chloro-palladium;dicyclohexyl-[3-(2,4,6-triisopropylphenyl)phenyl]phosphane (1.16 g, 1.48 mmol,) in dioxane (100 mL) and H₂O (5.0 mL) was stirred at 80° C. for 4 hrs. On completion, the mixture filtered and the filtrate was concentrated in vacuo. The residue was purified by reversed phase flash (0.1% FA condition) to give the title compound (2.30 g, 53% yield) as white solid. ¹H NMR (400 MHz, DMSO-d₆) δ 11.09 (s, 1H), 7.27 (s, 1H), 7.14-7.04 (m, 2H), 6.11 (s, 1H), 5.36 (dd, J=12.8, 5.2 Hz, 1H), 4.01 (d, J=7.2 Hz, 2H), 3.55 (t, J=5.6 Hz, 2H), 3.35 (s, 3H), 2.95-2.83 (m, 1H), 2.73-2.59 (m, 2H), 2.06-1.95 (m, 2H), 1.46-1.39 (m, 9H), 1.17 (t, J=7.2 Hz, 1H). LC-MS (ESI⁺) m/z 441.2 (M+H)⁺.

### Step 2—Tert-butyl 4-[1-(2,6-dioxo-3-piperidyl)-3-methyl-2-oxo-benzimidazol-5-yl]piperidine-1-carboxylate

To a solution of tert-butyl 4-[1-(2,6-dioxo-3-piperidyl)-3-methyl-2-oxo-benzimidazol-5-yl]-3,6-dihydro-2H-pyridine-1-carboxylate (2.30 g, 5.22 mmol) in THF (150 mL) was added Pd/C (800 mg, 10 wt %) and Pd(OH)₂ (800 mg, 5.70 mmol) at 25° C. The reaction mixture was stirred at 60° C. for 16 hr under H₂ (15 psi). On completion, the reaction mixture was filtered with celite and the filtrate was concentrated in vacuo to give the title compound (2.30 g, 87% yield) as white solid. ¹H NMR (400 MHz, CDCl₃) δ 8.10 (s, 1H), 7.02-6.87 (m, 2H), 6.76 (d, J=8.0 Hz, 1H), 5.23 (dd, J=5.6, 12.6 Hz, 1H), 4.30-4.25 (m, 2H), 3.45 (s, 3H), 2.99-2.68 (m, 6H), 2.30-2.21 (m, 1H), 1.88-1.81 (m, 2H), 1.51 (s, 9H), 1.48-1.44 (m, 2H). LC-MS (ESI⁺) m/z 465.2 (M+23)+. Step 3—3-[3-Methyl-2-oxo-5-(4-piperidyl)benzimidazol-1-yl]piperidine-2,6-dione

To a mixture of tert-butyl 4-[1-(2,6-dioxo-3-piperidyl)-3-methyl-2-oxo-benzimidazol-5-yl]piperidine-1-carboxylate (300 mg, 678 umol) in DCM (3.0 mL) was added HCl/dioxane (4 M, 170 uL) in one portion at 25° C. under N₂. The mixture was stirred at 25° C. for 30 min. On completion, the reaction mixture was concentrated in vacuo to give the title compound (250 mg, 910% yield, HCl salt) as white solid. LC-MS (ESI⁺) m/z 343.1 (M+H)⁺.

### Synthesis of 4-Benzylsulfanyl-2-methyl-aniline (Intermediate DE)

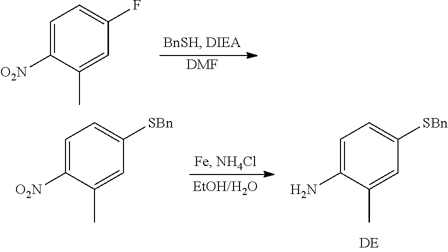

### Step-1—4-Benzylsulfanyl-2-methyl-1-nitro-benzene

A mixture of 4-fluoro-2-methyl-1-nitro-benzene (20.0 g, 128 mmol, CAS #446-33-3), BnSH (18.1 mL, 154 mmol), and DIEA (33.3 g, 257 mmol, 44.9 mL) in DMF (200 mL) was degassed and purged with N₂ for three times. Then the mixture was stirred at 80° C. for 16 hours under N₂ atmosphere. On completion, the reaction mixture was quenched with NaClO (10 mL) at 25° C., and then diluted with H₂O (10 mL) and extracted with EA (10 mL×3). The combined organic layers were washed with brine (10 mL×2), dried over anhydrous Na₂SO₄, filtered and concentrated in vacuo to give a residue. Then the residue was purified by column chromatography (SiO₂, Petroleum ether/Ethyl acetate=100/1 to 10/1) to give the title compound (26.0 g, 76% yield) as a yellow solid. ¹H NMR (400 MHz, CDCl₃) δ 8.05 (d, J=4.0 Hz, 1H) 7.51-7.37 (m, 5H) 7.30-7.25 (m, 2H) 4.34 (s, 2H) 2.69 (s, 3H). LC-MS (ESI⁺) m/z 260.0 (M+H)⁺.

### Step-2—4-Benzylsulfanyl-2-methyl-aniline

A mixture of 4-benzylsulfanyl-2-methyl-1-nitro-benzene (18.0 g, 69.4 mmol), Fe (23.2 g, 416 mmol), NH₄Cl (37.1 g, 694 mmol) in EtOH (180 mL) and H₂O (36 mL) was degassed and purged with N₂ for three times, and then the mixture was stirred at 80° C. for 1.5 hours under N₂ atmosphere. On completion, the reaction mixture was diluted with H₂O 100 mL and extracted with EA (60 mL×3). The combined organic layers were washed with brine (40 mL×2), dried over anhydrous Na₂SO₄, filtered and concentrated in vacuo to give a residue. The residue was purified by column chromatography (SiO₂, Petroleum ether/Ethyl acetate=100/1 to 10/1) to give the title compound (63 g, 98% yield) as a black oil. ¹H NMR (400 MHz, DMSO-d₆) δ 7.29-7.18 (m, 5H) 6.99 (s, 1H) 6.93 (d, J=1.6 Hz, 1H) 6.57 (d, J=8.0 Hz, 1H) 4.99 (s, 2H) 3.96 (s, 2H) 2.03 (s, 3H).

### Synthesis of Tert-butyl 4-but-3-enylpiperazine-1-carboxylate (Intermediate DJ)

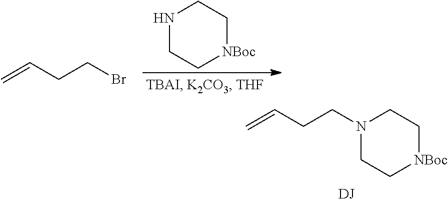

To a solution of 4-bromobut-1-ene (2.83 g, 20.9 mmol, CAS #5162-44-7) and tert-butylpiperazine-1-carboxylate hydrochloride (3.00 g, 13.4 mmol, CAS #57260-71-6) in THF (100 mL) was added K₂CO₃ (6.69 g, 48.4 mmol) and TBAI (300 mg, 812 umol). Then the mixture was stirred at 70° C. for 15 hrs. On completion, the mixture was filtered, diluted with water (100 mL) and extracted with EA (30 mL×3). The combined organic layer was dried over anhydrous Na₂SO₄, filtered and concentrated in vacuo to give the residue. The residue was purified by column chromatography (SiO₂, PE:EA=15:1 to 1:1) to give the title compound (1.50 g, 46% yield) as yellow oil. ¹H NMR (400 MHz, CDCl₃) δ 5.86-5.76 (m, 1H), 5.09-5.00 (m, 2H), 3.48-3.40 (m, 4H), 2.45-2.39 (m, 6H), 2.28-2.23 (m, 2H), 1.47 (s, 9H). LC-MS (ESI⁺) m/z 241.1 (M+H)⁺.

### Synthesis of 6-Chloro-8-cyclopentyl-2-[2-methyl-4-(4-piperazin-1-ylbutylsulfonyl)anilino]pyrido[2,3-d] pyrimidin-7-one (Intermediate DK)

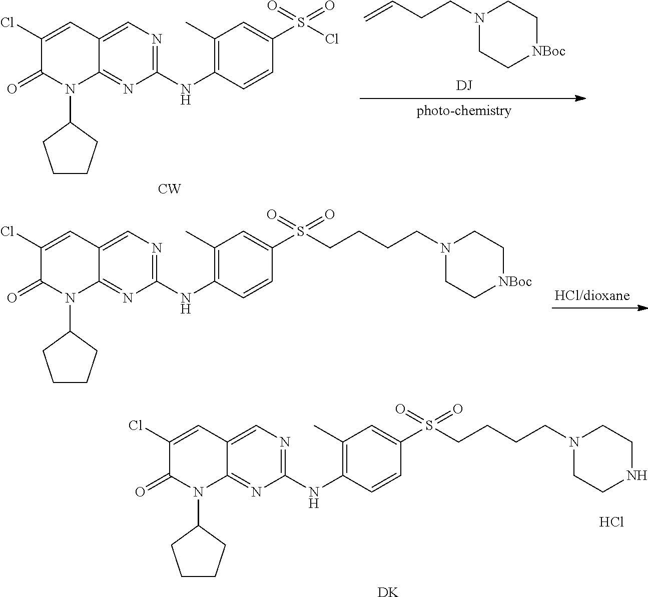

### Step 1—Tert-butyl 4-[4-[4-[(6-chloro-8-cyclopentyl-7-oxo-pyrido[2,3-d]pyrimidin-2-yl)amino]-3-methyl-phenyl]sulfonylbutyl]piperazine-1-carboxylate

An oven-dried 15 mL vial equipped with magnetic stir bar was charged with tert-butyl 4-but-3-enylpiperazine-1-carboxylate (80.0 mg, 332 umol, Intermediate DJ), 4-[(6-chloro-8-cyclopentyl-7-oxo-pyrido[2,3-d]pyrimidin-2-yl)amino]-3-methyl-benzenesulfonyl chloride (377 mg, 832 umol, Intermediate CW), IR(PPY)₃ (1.09 mg, 1.66 umol), 4-mercaptophenol (8.40 mg, 66.5 umol), bis(trimethylsilyl)silyl-trimethyl-silane (165 mg, 665 umol) in ACN (3 mL). The vial was sealed and placed under nitrogen and the reaction was stirred and irradiated with a 10 W blue LED lamp (3 cm away), with cooling water to keep the reaction temperature at 25° C. for 14 hrs. On completion, the mixture was filtered and concentrated in vacuo to give the residue. The residue was purified by prep-HPLC (column: Phenomenex luna C18 150*25 mm*10 um; mobile phase: [water (FA)-ACN]; B %: 27%-57%, 10 min) to give the title compound (20.0 mg, 9% yield) as white solid. LC-MS (ESI⁺) m/z 659.4 (M+H)⁺.

### Step 2—6-Chloro-8-cyclopentyl-2-[2-methyl-4-(4-piperazin-1-ylbutylsulfonyl)anilino]pyrido[2,3-d]pyrimidin-7-one

A solution of tert-butyl 4-[4-[4-[(6-chloro-8-cyclopentyl-7-oxo-pyrido[2,3-d]pyrimidin-2-yl)amino]-3-methyl-phenyl]sulfonylbutyl]piperazine-1-carboxylate (19.0 mg, 28.8 umol) in HCl/dioxane (1 mL) was stirred at 25° C. for 0.5 hr. On completion, the mixture was concentrated in vacuo to give the title compound (17.0 mg, 99% yield, HCl) as black-brown solid. LC-MS (ESI⁺) m/z 559.0 (M+H)⁺.

### Synthesis of 4-(1,3-dioxolan-2-yl)piperidine (Intermediate DL)

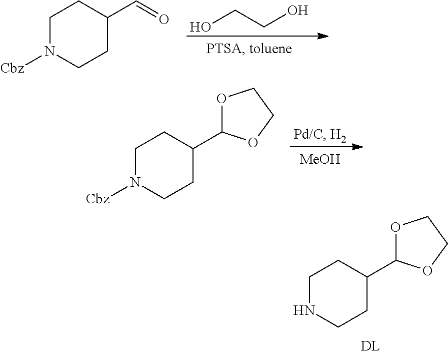

### Step 1—Benzyl 4-(1,3-dioxolan-2-yl)piperidine-1-carboxylate

A solution of benzyl 4-formylpiperidine-1-carboxylate (20.0 g, 80.9 mmol, CAS #138163-08-3), PTSA (1.4 g, 8.09 umol) and ethylene glycol (5.52 g, 88.9 mmol, CAS #107-21-1) in toluene (200 mL) was refluxed at 130° C. for 16 hrs. On completion, the reaction mixture was concentrated in vacuo. The residue was purified by column chromatography (SiO₂, Petroleum ether/Ethyl acetate=5/1 to 0/1) to give the title compound (15.0 g, 63% yield). ¹H NMR (400 MHz, CDCl₃) δ 7.43-7.28 (m, 5H), 5.13 (s, 2H), 4.65 (d, J=4.4 Hz, 1H), 4.24 (s, 2H), 3.99-3.82 (m, 4H), 2.77 (s, 2H), 1.85-1.64 (m, 3H), 1.42-1.26 (m, 2H).

### Step 2—4-(1,3-Dioxolan-2-yl)piperidine

To a solution of benzyl 4-(1,3-dioxolan-2-yl)piperidine-1-carboxylate (5 g, 20 mmol) in MeOH (100 mL) was added Pd/C (1.5 g, 1.4 mmol, 10 wt %) under N₂. The suspension was degassed in vacuo and purged with H₂ several times. The mixture was stirred at 25° C. for 4 hours under H₂ (15 PSI). On completion, the reaction mixture was filtered and concentrated in vacuo to give the title compound (2.3 g, 85% yield) as yellow solid. ¹H NMR (400 MHz, CDCl₃) δ 4.62 (d, J=4.8 Hz, 1H), 3.98-3.81 (m, 4H), 3.11 (d, J=12.0 Hz, 2H), 2.60 (m, 2H), 1.98 (s, 1H), 1.73 (d, J=14.4 Hz, 2H), 1.69-1.62 (m, 1H), 1.38-1.24 (m, 2H).

### Synthesis of 1-[1-(2,6-dioxo-3-piperidyl)-3-methyl-2-oxo-benzimidazol-5-yl]piperidine-4-carbaldehyde (Intermediate DM)

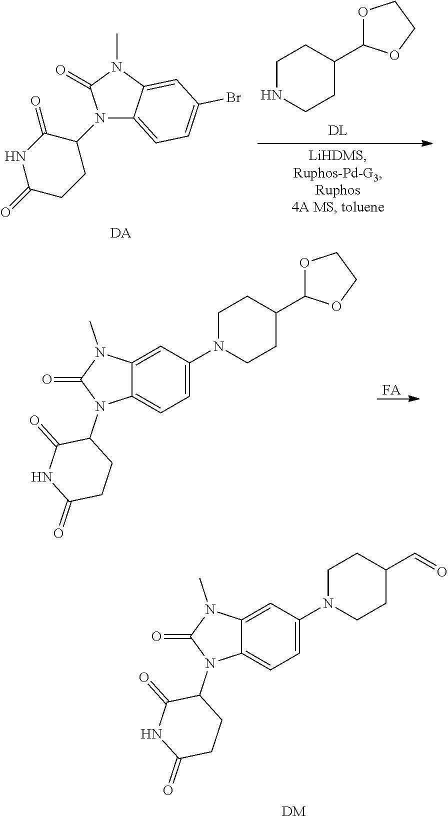

### Step 1—3-[5-[4-(1,3-Dioxolan-2-yl)-1-piperidyl]-3-methyl-2-oxo-benzimidazol-1-yl]piperidine-2,6-dione

To a solution of 4-(1,3-dioxolan-2-yl)piperidine (500 mg, 3.18 mmol, Intermediate DL) and 3-(5-bromo-3-methyl-2-oxo-benzimidazol-1-yl)piperidine-2,6-dione (1.08 g, 3.18 mmol, Intermediate DA) in toluene (15 mL) was added RuPhos (148 mg, 318 umol), RuPhos Pd G₃ (266 mg, 318 umol) and LiHMDS (1 M, 19.0 mL). On completion, the reaction mixture was acidified to pH=7 by HCOOH and concentrated in vacuo. The residue was triturated with PE/EA (3/1), filtered and the filter cake was triturated with water. The solid was filtered and dried in vacuo to give the title compound (1.1 g, 83% yield) as gray solid. ¹H NMR (400 MHz, DMSO-d₆) δ 11.05 (s, 1H), 6.92 (d, J=8.4 Hz, 1H), 6.82 (d, J=2.0 Hz, 1H), 6.63 (dd, J=2.0, 8.4 Hz, 1H), 5.28 (dd, J=5.2, 12.8 Hz, 1H), 4.61 (d, J=5.2 Hz, 1H), 3.93-3.75 (m, 4H), 3.62 (d, J=12.4 Hz, 2H), 3.37-3.30 (m, 3H), 2.95-2.82 (m, 1H), 2.67 (dd, J=4.4, 12.8 Hz, 1H), 2.63-2.55 (m, 3H), 2.02-1.93 (m, 1H), 1.78-1.72 (m, 2H), 1.65-1.56 (m, 1H), 1.50-1.39 (m, 2H).

### Step 2—1-[1-(2,6-Dioxo-3-piperidyl)-3-methyl-2-oxo-benzimidazol-5-yl]piperidine-4-carbaldehyde

A solution of 3-[5-[4-(1,3-dioxolan-2-yl)-1-piperidyl]-3-methyl-2-oxo-benzimidazol-1-yl]piperidine-2,6-dione (100 mg, 241 umol) in HCOOH (3 mL) was stirred at 50° C. for 3 hrs. On completion, the reaction mixture was concentrated in vacuo to give the title compound (89 mg, 99% yield) as a brown oil. LCMS (ESI⁺) m/z 371.0 (M+H)⁺.

### Synthesis of N-(4-Benzylsulfanyl-2-methyl-phenyl)-4-chloro-5-(trifluoromethyl)pyrimidin-2-amine (Intermediate EA)

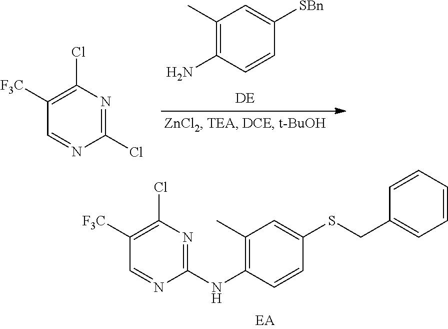

To a solution of 2,4-dichloro-5-(trifluoromethyl)pyrimidine (500 mg, 2.30 mmol, CAS #3932-97-6) in mixture solvent of DCE (6 mL) and t-BuOH (6 mL) was added ZnCl₂ (1 M, 2.77 mL) at 0° C. After 1 hour, a solution of 4-benzylsulfanyl-2-methyl-aniline (528 mg, 2.30 mmol, Intermediate DE) and TEA (256 mg, 2.5 mmol) in mixture solvent of DCE (3 mL) and t-BuOH (3 mL) was added dropwise into the above solution. The mixture was then stirred at 25° C. for 16 hrs. On completion, the mixture was diluted with H₂O (20 mL) and extracted with EA (20 mL×3). The combined organic layers were washed with saturated NaCl (10 mL), dried over anhydrous Na₂SO₄, filtered and concentrated in vacuo to give a residue. The residue was purified by column chromatography (SiO₂, PE:EA=50:1 to 20:1) to give the title compound (600 mg, 63% yield) as a yellow solid. ¹H NMR (400 MHz, DMSO-d₆) δ 10.04 (s, 1H), 8.66-8.60 (m, 1H), 7.40-7.35 (m, 2H), 7.31-7.28 (m, 2H), 7.28-7.21 (m, 3H), 7.20-7.16 (m, 1H), 4.24 (s, 2H), 2.15 (s, 3H). LC-MS (ESI⁺) m/z 410.0 (M+H)⁺.

### Synthesis of 3-Methyl-4-[[4-(1-methylpyrazol-4-yl)-5-(trifluoromethyl)pyrimidin-2-yl]amino]benzenesulfonyl chloride (Intermediate EB)

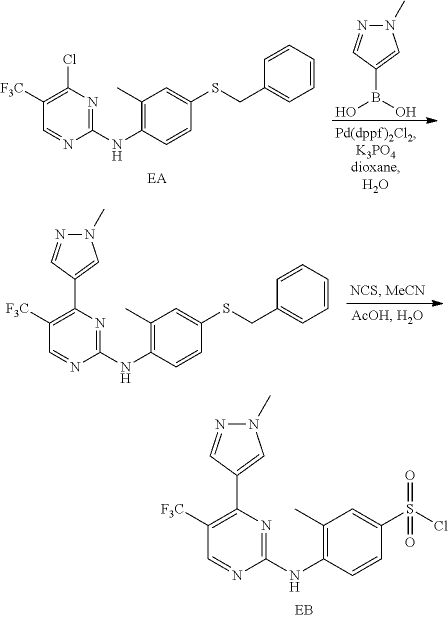

### Step 1—N-(4-benzylsulfanyl-2-methyl-phenyl)-4-(1-methylpyrazol-4-yl)-5-(trifluoromethyl) pyrimidin-2-amine

To a solution of N-(4-benzylsulfanyl-2-methyl-phenyl)-4-chloro-5-(trifluoromethyl)pyrimidin-2-amine (2.00 g, 4.88 mmol, Intermediate EA) and (1-methylpyrazol-4-yl)boronic acid (921 mg, 7.32 mmol, CAS #847818-55-7) in dioxane (25 mL) and H₂O (5 mL) was added K₃PO₄ (3.11 g, 14.6 mmol) and Pd(dppf)Cl₂·CH₂Cl₂ (398 mg, 487 umol). Then the mixture was purged with N₂ three times and stirred at 80° C. for 10 hrs. On completion, the mixture was filtered and concentrated in vacuo to give a residue. The residue was purified by column chromatography (SiO₂, PE:EA=10:1 to 5:1) to give the title compound (1.20 g, 54% yield) as a yellow solid. ¹H NMR (400 MHz, DMSO-d₆) δ 9.44 (s, 1H), 8.62 (s, 1H), 8.15 (s, 1H), 7.85 (s, 1H), 7.40-7.34 (m, 3H), 7.31 (t, J=7.4 Hz, 2H), 7.27-7.17 (m, 3H), 4.23 (s, 2H), 3.93 (s, 3H), 2.19 (s, 3H). LC-MS (ESI⁺) m/z 456.4 (M+H)⁺.

### Step 2—3-Methyl-4-[[4-(1-methylpyrazol-4-yl)-5-(trifluoromethyl)pyrimidin-2-yl]amino] benzenesulfonyl chloride

To a solution of N-(4-benzylsulfanyl-2-methyl-phenyl)-4-(1-methylpyrazol-4-yl)-5-(trifluoromethyl) pyrimidin-2-amine (1.00 g, 2.20 mmol) in ACN (12 mL), AcOH (1.2 mL) and H₂O (0.2 mL) was added NCS (1.03 g, 7.68 mmol), then the mixture was stirred at 25° C. for 0.5 hr. On completion, the mixture was concentrated in vacuo to give the residue. The residue was purified by column chromatography (SiO₂, PE:EA=10:1 to 3:1) to give the title compound (700 mg, 73% yield) as a brown solid. ¹H NMR (400 MHz, DMSO-d₆) δ 9.45 (s, 1H), 8.62 (s, 1H), 8.16 (s, 1H), 7.86 (s, 1H), 7.50-7.47 (m, 1H), 7.46-7.38 (m, 2H), 3.92 (s, 3H), 2.23 (s, 3H). LC-MS (ESI⁺) m/z 432.0 (M+H)⁺.

### Synthesis of N-[2-methyl-4-(4-piperazin-1-ylbutylsulfonyl)phenyl]-4-(1-methylpyrazol-4-yl)-5-(trifluoromethyl)pyrimidin-2-amine (Intermediate EC)

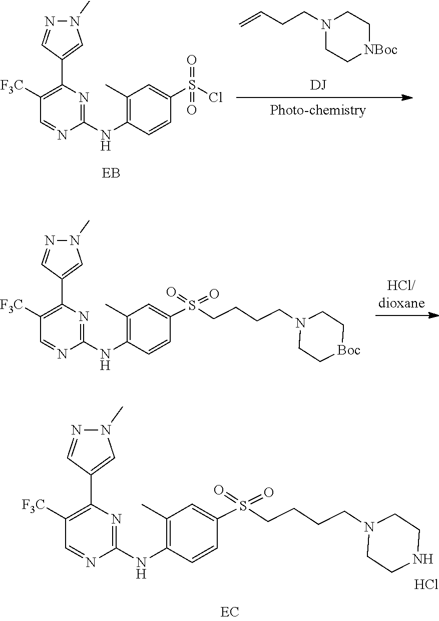

### Step 1—Tert-butyl 4-[4-[3-methyl-4-[[4-(1-methylpyrazol-4-yl)-5-(trifluoromethyl)pyrimidin-2-yl]amino]phenyl]sulfonylbutyl]piperazine-1-carboxylate

To a solution of 3-methyl-4-[[4-(1-methylpyrazol-4-yl)-5-(trifluoromethyl)pyrimidin-2-yl]amino]benzenesulfonyl chloride (300 mg, 694 umol, Intermediate EB) and tert-butyl 4-but-3-enylpiperazine-1-carboxylate (60.0 mg, 249 umol, Intermediate DJ) in ACN (3 mL) was added IR(PPY)₃ (2.27 mg, 3.47 umol), (TMS)₃SiH (1.39 mmol) and 4-mercaptophenol (17.5 mg, 138 umol). Then the mixture was stirred and irradiated with a 10 W blue LED lamp (3 cm away), with cooling water to keep the reaction temperature at 25° C. for 14 hrs. On completion, the mixture was filtered to give the residue. The residue was purified by prep-HPLC (column: Phenomenex luna C18 150*25 mm*10 um; mobile phase: [water (FA)-ACN]; B %: %-%, 15 min) to give the title compound (70.0 mg, 44% yield) as a yellow solid. LC-MS (ESI⁺) m/z 638.2 (M+H)⁺.

### Step 2—N-[2-methyl-4-(4-piperazin-1-ylbutylsulfonyl)phenyl]-4-(1-methylpyrazol-4-yl)-5-(trifluoromethyl)pyrimidin-2-amine

To a solution of tert-butyl 4-[4-[3-methyl-4-[[4-(1-methylpyrazol-4-yl)-5-(trifluoromethyl)pyrimidin -2-yl]amino]phenyl]sulfonylbutyl]piperazine-1-carboxylate (40.0 mg, 62.7 umol) was added HCl/dioxane (2 mL), then the mixture was stirred at 25° C. for 1 hr. On completion, the mixture was concentrated in vacuo to give the title compound (35.0 mg, 97% yield, HCl) as a white solid. LC-MS (ESI⁺) m/z 538.1 (M+H)⁺.

### Synthesis of 5-Bromo-2-chloro-N-isopropyl-pyrimidin-4-amine (Intermediate DF)

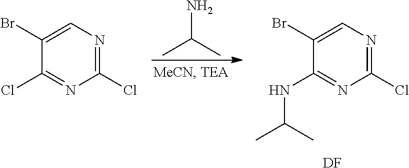

To a solution of 5-bromo-2,4-dichloro-pyrimidine (10.0 g, 43.8 mmol, 5.62 mL, CAS #36082-50-5) in ACN (250 mL) was added TEA (5.77 g, 57.0 mmol, 7.94 mL) and propan-2-amine (3.37 g, 57.0 mmol, 4.90 mL) at 0° C. for 30 min. Then the mixture was stirred for 15.5 hours at 25° C. On completion, the reaction mixture was diluted with H₂O (200 mL) and extracted with EA (3×100 mL). The combined organic layers were washed with brine (2×100 mL), dried over Na₂SO₄, filtered and concentrated under reduced pressure to give the title compound (10 g, 90% yield) as a white solid. LC-MS (ESI⁺) m/z 251.8 (M+H)⁺.

### Synthesis of 8-isopropyl-2-methylsulfanyl-pyrido[2,3-d]pyrimidin-7-one (Intermediate DN)

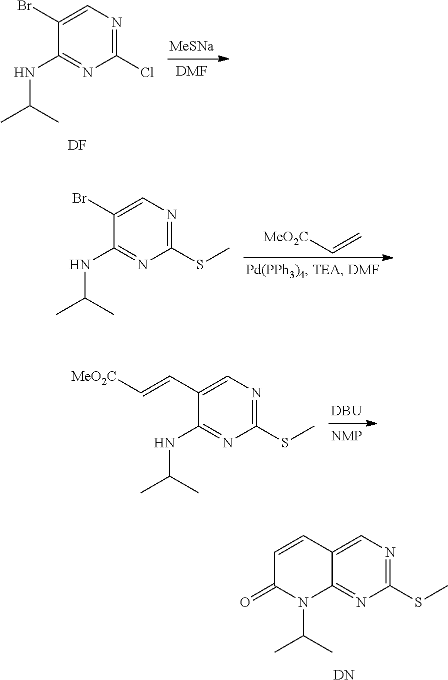

### Step 1—5-Bromo-N-isopropyl-2-methylsulfanyl-pyrimidin-4-amine

To a solution of 5-bromo-2-chloro-N-isopropyl-pyrimidin-4-amine (10.0 g, 39.9 mmol, Intermediate DF) in DMF (110 mL) was added NaSMe (7.12 g, 101 mmol, 6.47 mL). The mixture was stirred at 25° C. for 16 hrs under N₂. On completion, the reaction mixture was quenched with H₂O (100 mL) at 25° C., and then extracted with EA (100 mL×3). The combined organic layers were washed with brine (100 mL×2), dried over anhydrous Na₂SO₄, filtered and concentrated in vacuo to give the title compound (9.50 g, 90% yield) as a yellow solid. ¹H NMR (400 MHz, DMSO-d₆) δ 8.12 (s, 1H), 7.95 (s, 1H), 4.32-4.25 (m, 1H), 2.89 (s, 3H), 2.73 (s, 3H), 2.41 (s, 3H). LC-MS (ESI⁺) m/z 263.8 (M+H)⁺.

### Step 2—Methyl (E)-3-[4-(isopropylamino)-2-methylsulfanyl-pyrimidin-5-yl]prop-2-enoate

A mixture of 5-bromo-N-isopropyl-2-methylsulfanyl-pyrimidin-4-amine (9.50 g, 36.2 mmol), methyl prop-2-enoate (22.3 g, 259 mmol, 23.3 mL, CAS #96-33-3), Pd(PPh₃)₄ (4.19 g, 3.62 mmol), and TEA (11.0 g, 108 mmol, 15.0 mL) in DMF (100 mL) was degassed and purged with N₂ three times. Then the mixture was stirred at 90° C. for 32 hrs under N₂ atmosphere. On completion, the reaction mixture was quenched with H₂O (100 mL) at 25° C., and then extracted with EA (100 mL×3). The combined organic layers were washed with brine (100 mL×2), dried over anhydrous Na₂SO₄, filtered and concentrated in vacuo to give a residue. The residue was purified by column chromatography (SiO₂, Petroleum ether/Ethyl acetate=10/1 to 3/1) (Rf=0.40, PE:EA=1:1) to give the title compound (5.80 g, 59% yield) as a yellow solid. ¹H NMR (400 MHz, DMSO-d₆) δ 8.43-8.30 (m, 1H), 7.79 (d, J=15.6 Hz, 1H), 7.49 (d, J=7.2 Hz, 1H), 6.55-6.43 (m, 1H), 4.35 (d, J=6.8, 13.4 Hz, 1H), 3.71 (s, 3H), 2.44 (s, 3H), 1.19 (d, J=6.4 Hz, 6H). LC-MS (ESI⁺) m/z 268.1 (M+H)⁺.

### Step 3-8-Isopropyl-2-methylsulfanyl-pyrido[2,3-d]pyrimidin-7-one

A mixture of methyl (E)-3-[4-(isopropylamino)-2-methylsulfanyl-pyrimidin-5-yl]prop-2-enoate (5.73 g, 21.4 mmol), DBU (16.3 g, 107 mmol, 16.1 mL) in NMP (50.0 mL) was degassed and purged with N₂ three times. Then the mixture was stirred at 120° C. for 1 hr under N₂ atmosphere. On completion, the mixture was diluted with H₂O (300 mL), and extracted with DCM (3×100 mL). The combined organic layer was washed with brine (3×100 mL), then dried with anhydrous Na₂SO₄, filtered and the filtrate was concentrated in vacuo. The mixture was purified by reversed phase (0.1% FA) to give the title compound (4.20 g, 83% yield) as a white solid. ¹H NMR (400 MHz, DMSO-d₆) δ 8.84 (s, 1H), 7.86 (d, J=9.6 Hz, 1H), 6.56 (d, J=9.6 Hz, 1H), 5.75-5.56 (m, 1H), 2.59 (s, 3H), 1.53 (d, J=6.8 Hz, 6H). LC-MS (ESI⁺) m/z 236.1 (M+H)⁺.

### Synthesis of 4-[[6-(Difluoromethyl)-8-isopropyl-7-oxo-pyrido[2,3-d]pyrimidin-2-yl]amino]-3-methyl-benzenesulfonyl chloride (Intermediate DO)

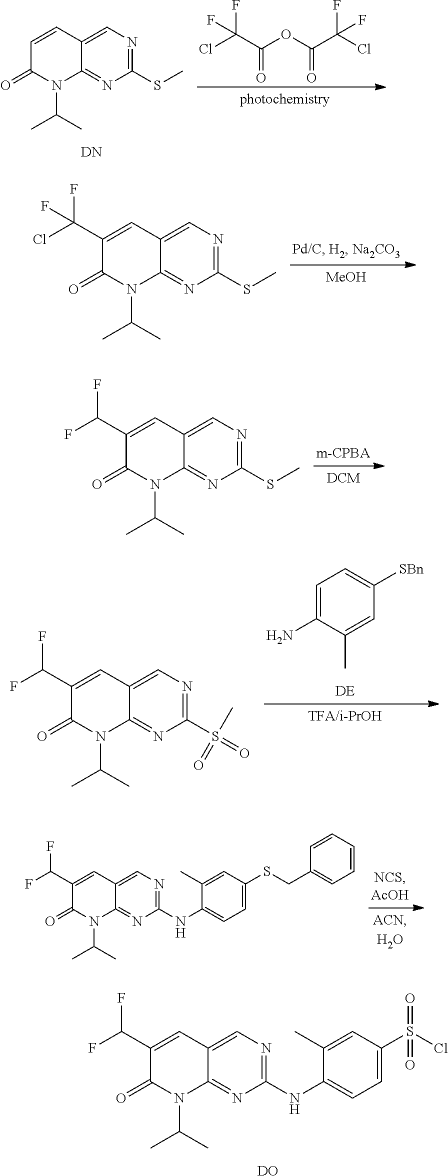

### Step 1—6-[Chloro(difluoro)methyl]-8-isopropyl-2-methylsulfanyl-pyrido[2,3-d]pyrimidin-7-one

To an 15 mL vial equipped with a stir bar was added 4-phenylpyridine N-Oxide (3.64 g, 21.0 mmol), 8-isopropyl-2-methylsulfanyl-pyrido[2,3-d]pyrimidin-7-one (2.00 g, 8.50 mmol, Intermediate DN), and Ru(bpy)₃Cl₂·6H₂O (63.6 mg, 85.0 umol) in dry ACN (20 mL), then (2-chloro-2,2-difluoro-acetyl) 2-chloro -2,2-difluoro-acetate (5.16 g, 21.0 mmol, CAS #2834-28-3) was added. The vial was sealed and placed under nitrogen was added. The reaction was stirred and irradiated with a 34 W blue LED lamp (2 cm away), with cooling water to keep the reaction temperature at 25° C. for 16 hrs. On completion, the mixture was concentrated in vacuo. The residue was purified by column chromatography (SiO₂, Petroleum ether/Ethyl acetate=20/1 to 10/1) (Rf=0.55, PE:EA=1:1) to give the title compound (1.37 g, 50% yield) as a yellow solid. ¹H NMR (400 MHz, DMSO-d₆) δ 9.02 (s, 1H), 8.50 (s, 1H), 5.82-5.64 (m, 1H), 2.63 (s, 3H), 1.57 (d, J=6.8 Hz, 6H). LC-MS (ESI⁺) m/z 319.6 (M+H)⁺.

### Step 2—6-(Difluoromethyl)-8-isopropyl-2-methylsulfanyl-pyrido[2,3-d]pyrimidin-7-one

A mixture of 6-[chloro(difluoro)methyl]-8-isopropyl-2-methylsulfanyl-pyrido[2,3-d]pyrimidin-7-one (200 mg, 625 umol), Pd/C (10.0 mg, 6.25 umol, 10 wt %), Na₂CO₃ (99.0 mg, 938 umol) in THF (2 mL) was degassed and purged with H₂ three times. Then the mixture was stirred at 25° C. for 2 hours under H₂ atmosphere. On completion, the mixture was filtered, and the filtrate was concentrated in vacuo. The residue was purified by column chromatography (SiO₂, Petroleum ether/Ethyl acetate=30/1 to 10/1) (Rf=0.70, PE:EA=3:1) to give the title compound (70.0 mg, 39% yield) as a white solid. ¹H NMR (400 MHz, DMSO-d₆) δ 9.00 (s, 1H), 8.28 (s, 1H), 5.87-5.59 (m, 1H), 3.36-3.26 (m, 1H), 2.62 (s, 3H), 1.56 (d, J=6.8 Hz, 6H). LC-MS (ESI⁺) m/z 286.0 (M+H)⁺.

### Step 3—6-(Difluoromethyl)-8-isopropyl-2-methylsulfonyl-pyrido[2,3-d]pyrimidin-7-one

To a solution of 6-(difluoromethyl)-8-isopropyl-2-methylsulfanyl-pyrido[2,3-d]pyrimidin-7-one (260 mg, 911 umol) in DCM (2 mL) was added m-CPBA (740 mg, 3.65 mmol, 85% solution). The mixture was stirred at 40° C. for 3 hrs. On completion, the mixture was quenched with NaHCO₃ (10 mL), then extracted with EA (3×10 mL). The combined organic layers were washed with brine (3×10 mL), dried with anhydrous Na₂SO₄, filtered and the filtrate was concentrated in vacuo. The residue was purified by column chromatography (SiO₂, Petroleum ether/Ethyl acetate=30/1 to 10/1) to give the title compound (100 mg, 34% yield) as a yellow solid. LC-MS (ESI⁺) m/z 317.9 (M+H)⁺.

### Step 4—2-(4-Benzylsulfanyl-2-methyl-anilino)-6-(difluoromethyl)-8-isopropyl-pyrido[2,3-d]pyrimidin-7-one

A mixture of 6-(difluoromethyl)-8-isopropyl-2-methylsulfonyl-pyrido[2,3-d]pyrimidin-7-one (70.0 mg, 220 umol), 4-benzylsulfanyl-2-methyl-aniline (151 mg, 661 umol, Intermediate DE), TFA (251 mg, 2.21 mmol, 163 uL) in IPA (2 mL), and then the mixture was stirred at 90° C. for 5 hrs. On completion, the mixture was concentrated in vacuo. The mixture was purified by reversed phase (0.1% FA) to give the title compound (27.0 mg, 26% yield) as a brown oily liquid. ¹H NMR (400 MHz, DMSO-d₆) δ 9.56 (s, 1H), 8.81 (s, 1H), 8.10 (s, 1H), 7.37-7.17 (m, 8H), 6.88 (t, J=56.0 Hz, 1H), 5.59-5.37 (m, 1H), 4.23 (s, 2H), 2.17 (s, 3H), 1.34 (s, 6H). LC-MS (ESI⁺) m/z 467.2 (M+H)⁺.

### Step 5—4-[[6-(Difluoromethyl)-8-isopropyl-7-oxo-pyrido[2,3-d]pyrimidin-2-yl]amino]-3-methyl-benzenesulfonyl chloride

To a solution of 2-(4-benzylsulfanyl-2-methyl-anilino) -6-(difluoromethyl)-8-isopropyl-pyrido[2,3-d] pyrimidin-7-one (22.0 mg, 47.1 umol) in ACN (1 mL), AcOH (0.1 mL), and H₂O (0.01 mL) was added NCS (16.0 mg, 126 umol). The mixture was stirred at 25° C. for 1 hr in the dark. On completion, the mixture was diluted with H₂O (10 mL), and extracted with EA (3×10 mL). The combined organic layer was washed with brine (3×10 mL), dried with anhydrous Na₂SO₄, filtered and the filtrate was concentrated in vacuo to give the title compound (20.0 mg, 95% yield) as a brown oily liquid. LC-MS (ESI⁺) m/z 442.9 (M+H)⁺.

### Synthesis of 4-[3-[4-[[6-(Difluoromethyl)-8-isopropyl-7-oxo-pyrido[2,3-d]pyrimidin-2-yl]amino]-3-methyl-phenyl]sulfonylpropoxy]cyclohexanecarbaldehyde (Intermediate KR)

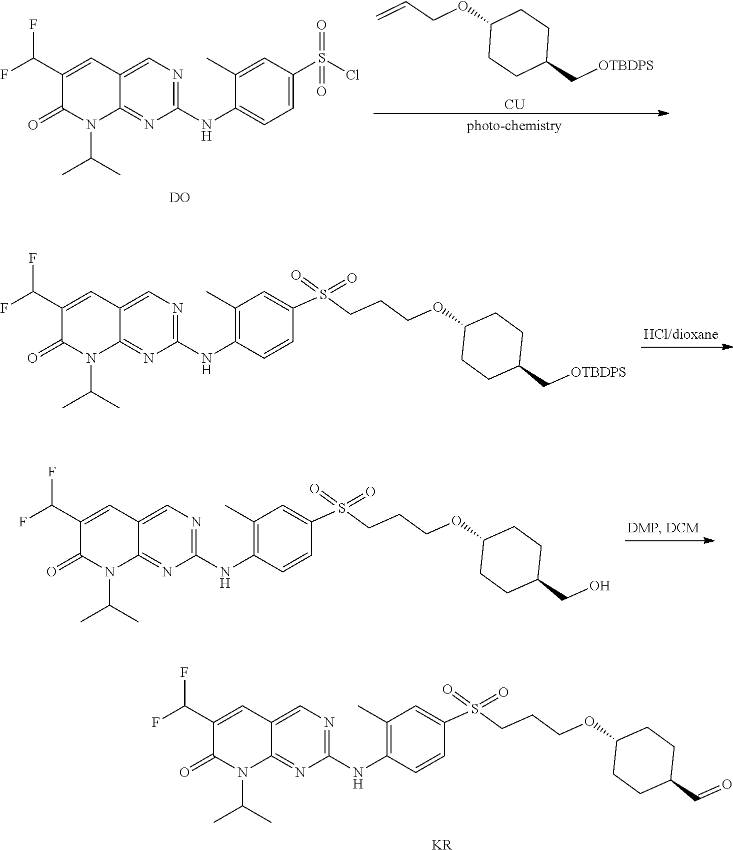

### Step 1—2-[4-[3-[4-[[Tert-butyl(diphenyl)silyl]oxymethyl]cyclohexoxy]propylsulfonyl]-2-methyl-anilino]-6-(difluoromethyl)-8-isopropyl-pyrido[2,3-d]pyrimidin-7-one

An oven-dried 15 mL vial equipped with magnetic stir bar was charged with (4-allyloxycyclohexyl)methoxy-tert-butyl-diphenyl-silane (140 mg, 342 umol, Intermediate CU), 4-[[6-(difluoromethyl)-8-isopropyl-7-oxo-pyrido[2,3-d]pyrimidin-2-yl]amino]-3-methyl-benzenesulfonyl chloride (379 mg, 856 umol, Intermediate DO), Ir(ppy)₃ (1.12 mg, 1.71 umol), (TMS)₃SiH (249 mg, 685 umol), and 4-mercaptophenol (8.65 mg, 68.5 umol) in ACN (3 mL, 0.167 M). The vial was sealed and placed under nitrogen was added. The reaction was stirred and irradiated with a 10 W blue LED lamp (3 cm away), with cooling water to keep the reaction temperature at 25° C. for 14 hrs. On completion, the mixture was concentrated in vacuo. The residue was purified by prep-TLC (SiO₂, PE:EA=3:1) (Rf=0.28, PE:EA=3:1) to give the title compound (220 mg, 78% yield) as a white solid. LC-MS (ESI⁺) m/z 817.5 (M+H)⁺.

### Step 2—6-(Difluoromethyl)-2-[4-[3-[4-(hydroxymethyl)cyclohexoxy]propylsulfonyl]-2-methyl-anilino]-8-isopropyl-pyrido[2,3-d]pyrimidin-7-one

To a solution of 2-[4-[3-[4-[[tert-butyl(diphenyl)silyl]oxymethyl] cyclohexoxy]propylsulfonyl]-2-methyl-anilino]-6-(difluoromethyl)-8-isopropyl-pyrido[2,3-d]pyrimidin-7-one (80.0 mg, 97.9 umol) in HCl/dioxane (1 mL). The mixture was stirred at 25° C. for 16 hrs. On completion, the mixture was concentrated in vacuo. The residue was purified by prep-TLC (SiO₂, PE:EA=3:1) (Rf=0.46, PE:EA=3:1) to give the title compound (58.0 mg, 96% yield, HCl) as a white solid. LC-MS (ESI⁺) m/z 579.3 (M+H)⁺.

### Step 3—4-[3-[4-[[6-(Difluoromethyl)-8-isopropyl-7-oxo-pyrido[2,3-d]pyrimidin-2-yl]amino]-3-methyl-phenyl]sulfonylpropoxy]cyclohexanecarbaldehyde

To a solution of 6-(difluoromethyl)-2-[4-[3-[4-(hydroxymethyl)cyclohexoxy] propylsulfonyl]-2-methyl-anilino]-8-isopropyl-pyrido[2,3-d]pyrimidin-7-one (58.0 mg, 94.2 umol, HCl) in DCM (1.5 mL) was added DMP (59.9 mg, 141 umol, 43.7 uL). The mixture was stirred at 25° C. for 1 hr. On completion, the mixture was quenched with Na₂S₂O₃ (3 mL), then diluted with H₂O (10 mL), and extracted with DCM (3×10 mL). The combined organic layer was washed with brine (3×10 mL), dried with anhydrous Na₂SO₄, filtered and the filtrate was concentrated in vacuo to give the title compound (50.0 mg, 91% yield) as a white solid. LC-MS (ESI⁺) m/z 577.2 (M+H)⁺.

### Synthesis of 4-[(6-Chloro-8-isopropyl-7-oxo-pyrido[2,3-d]pyrimidin-2-yl)amino]-3-methyl-benzenesulfonyl chloride (Intermediate DG)

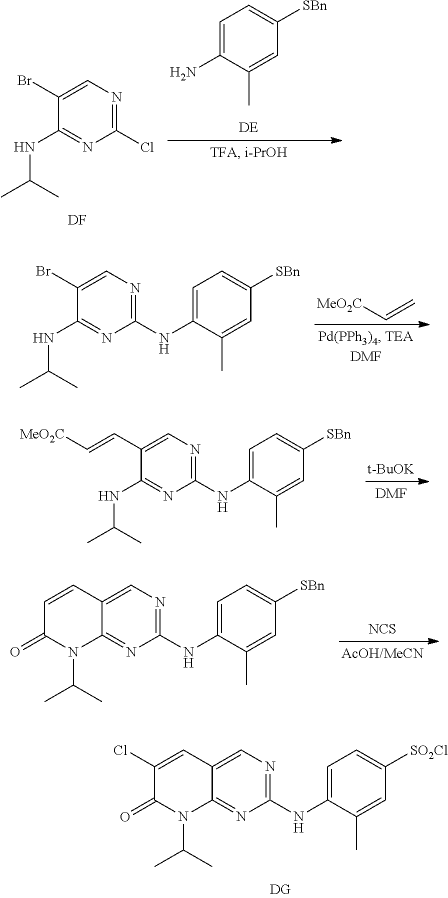

### Step 1—N2-(4-benzylsulfanyl-2-methyl-phenyl)-5-bromo-N4-isopropyl-pyrimidine-2,4-diamine

To a solution of 4-benzylsulfanyl-2-methyl-aniline (1.00 g, 4.36 mmol, Intermediate DE) in IPA (10 mL) was added 5-bromo-2-chloro-N-isopropyl-pyrimidin-4-amine (1.20 g, 4.80 mmol, Intermediate DF) and TFA (9.94 g, 87.2 mmol, 6.46 mL) at 20° C. under nitrogen flow. Then the reaction was stirred at 80° C. for 20 hrs under nitrogen atmosphere. On completion, the mixture was diluted with H₂O (20 mL), and extracted with EA (30 mL×3). The combined organic layer was washed with NaHCO₃ (30 mL), then washed with brine (30 mL×3), dried with anhydrous Na₂SO₄, filtered and the filtrate was concentrated in vacuo. The mixture was purified by pre-HPLC (column: Phenomenex luna C18 250*50 mm*15 um; mobile phase: [water(FA)-ACN]; B %: 23%-53%, 20 min) to give the title compound (600 mg, 31% yield) as a black solid. ¹H NMR (400 MHz, DMSO-d₆) δ 8.25 (s, 1H), 7.91 (s, 1H), 7.47 (d, J=8.4 Hz, 1H), 7.36-7.25 (m, 4H), 7.23 (d, J=6.8 Hz, 1H), 7.16 (s, 1H), 7.09 (d, J=8.4 Hz, 1H), 6.33 (d, J=8.0 Hz, 1H), 4.17 (s, 3H), 2.16 (s, 3H), 1.15 (d, J=6.4 Hz, 6H). LC-MS (ESI⁺) m/z 444.9 (M+H)⁺.

### Step 2—Methyl (E)-3-[2-(4-benzylsulfanyl-2-methyl-anilino)-4-(isopropylamino)pyrimidin-5-yl]prop-2-enoate

A mixture of N2-(4-benzylsulfanyl-2-methyl-phenyl)-5-bromo-N4-isopropyl-pyrimidine-2,4-diamine (2.20 g, 4.96 mmol), TEA (1.51 g, 14.8 mmol, 2.07 mL), and Pd(PPh₃)₄ (1.15 g, 992 umol) in DMF (25 mL) was added methyl prop-2-enoate (3.11 g, 36.1 mmol, 3.25 mL). The mixture was degassed and purged with N₂ three times, and then the mixture was stirred at 90° C. for 16 hours under N₂ atmosphere. On completion, the reaction mixture was quenched with H₂O (20 mL) at 25° C., then and extracted with EA (20 mL×3). The combined organic layers were washed with brine (20 mL×3), dried over anhydrous Na₂SO₄ filtered and the filtrate was concentrated under reduced pressure to give a residue. The residue was purified by column chromatography (SiO₂, Petroleum ether/Ethyl acetate=20/1 to 5/1) (Rf=0.5, PE:EA=2:1) to give the title compound (1.3 g, 58% yield) as a yellow solid. ¹H NMR (400 MHz, DMSO-d₆) δ 8.50 (s, 1H), 8.31 (s, 1H), 7.78 (d, J=15.6 Hz, 1H), 7.50 (d, J=8.4 Hz, 1H), 7.37-7.08 (m, 9H), 6.31 (d, J=15.2 Hz, 1H), 4.18 (s, 2H), 3.68 (s, 3H), 2.18 (s, 3H), 1.16-1.10 (m, 6H). LC-MS (ESI⁺) m/z 449.5 (M+H)⁺.

### Step 3—2-(4-Benzylsulfanyl-2-methyl-anilino)-8-isopropyl-pyrido[2,3-d]pyrimidin-7-one

To a solution of methyl (E)-3-[2-(4-benzylsulfanyl-2-methyl-anilino)-4-(isopropylamino) pyrimidin -5-yl]prop-2-enoate (110 mg, 245 umol) in DMF (2 mL) was added t-BuOK (82.5 mg, 735 umol). The mixture was stirred at 25° C. for 30 min. Then the mixture was heated to 120° C. and stirred for 1 hr. On completion, the mixture was concentrated in vacuo. The residue was purified by prep-TLC (SiO₂, PE:EA=1:1) (Rf=0.5, PE:EA=1:1) to give the title compound (50 mg, 48% yield) as a yellow solid. ¹H NMR (400 MHz, DMSO-d₆) δ 9.27 (s, 1H), 8.64 (s, 1H), 7.70 (d, J=9.2 Hz, 1H), 7.38-7.16 (m, 8H), 6.25 (d, J=9.2 Hz, 1H), 5.61-5.42 (m, 1H), 4.22 (s, 2H), 2.17 (s, 3H), 1.34 (d, J=5.2 Hz, 6H). LC-MS (ESI⁺) m z 867.3 (M+H)⁺.

### Step 4—4-[(6-Chloro-8-isopropyl-7-oxo-pyrido[2,3-d]pyrimidin-2-yl)amino]-3-methyl-benzenesulfonyl chloride

To a solution of 2-(4-benzylsulfanyl-2-methyl-anilino)-8-isopropyl-pyrido[2,3-d]pyrimidin-7-one (100 mg, 240 umol) in ACN (1 mL), AcOH (0.1 mL), H₂O (0.01 mL) was added NCS (128 mg, 960 umol). The mixture was stirred at 25° C. for 16 hrs in the dark. On completion, the mixture was quenched with H₂O (5 mL), and extracted with DCM (10 mL×3). The combined organic layer was washed with brine (10 mL×3), dried with anhydrous Na₂SO₄, filtered and the filtrate was concentrated in vacuo. The residue was purified by prep-TLC (SiO₂, PE:EA=1:1)(Rf=0.56, PE:EA=1:1) to give the title compound (35 mg, 34% yield) as a yellow solid. LC-MS (ESI⁺) m/z 426.8 (M+H)⁺.

### Synthesis of 2-[4-(Azetidin-3-ylmethylsulfonyl)-2-methyl-anilino]-6-chloro-8-isopropyl-pyrido[2,3-d]pyrimidin-7-one (Intermediate KS)

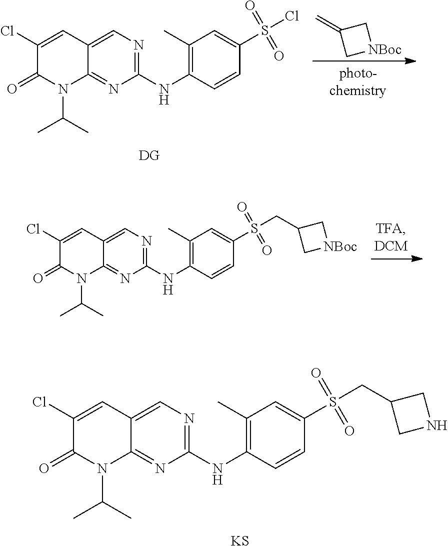

### Step 1—Tert-butyl 3-[[4-[(6-chloro-8-isopropyl-7-oxo-pyrido[2,3-d]pyrimidin-2-yl)amino]-3-methyl-phenyl]sulfonylmethyl]azetidine-1-carboxylate

To a solution of 4-[(6-chloro-8-isopropyl-7-oxo-pyrido[2,3-d]pyrimidin-2-yl)amino]-3-methyl-benzenesulfonyl chloride (700 mg, 1.64 mmol, Intermediate DG) and tert-butyl 3-methyleneazetidine-1-carboxylate (110 mg, 655 umol, CAS #934664-41-2) in ACN (1 mL) was added IR(PPY)₃ and TTMSS (162 mg, 655 umol). The mixture was stirred at 25° C. for 14 hrs. On completion, the mixture was filtered and concentrated in vacuo to give a residue. The residue was purified by prep-HPLC (column: Phenomenex luna C18 150*25 mm*10 um; mobile phase: [water (FA)-ACN]; B %: 53%-83%, 10 min) to give the title compound (350 mg, 46% yield) as a white solid. ¹H NMR (400 MHz, DMSO-d₆) δ 9.70 (s, 1H), 8.76 (s, 1H), 8.18 (s, 1H), 7.88 (d, J=8.4 Hz, 1H), 7.77 (d, J=2.0 Hz, 1H), 7.70 (dd, J=2.0, 8.4 Hz, 1H), 5.76-5.55 (m, 1H), 3.94-3.74 (m, 2H), 3.73-3.45 (m, 4H), 2.87-2.74 (m, 1H), 2.38 (s, 3H), 1.44 (d, J=6.8 Hz, 6H), 1.34 (s, 9H). LC-MS (ESI⁺) m/z 562.0 (M+H)⁺.

### Step 2—2-[4-(Azetidin-3-ylmethylsulfonyl)-2-methyl-anilino]-6-chloro-8-isopropyl-pyrido[2,3-d]pyrimidin-7-one

To a solution of tert-butyl 3-[[4-[(6-chloro-8-isopropyl-7-oxo-pyrido[2,3-d]pyrimidin-2-yl)amino]-M3-methyl-phenyl]sulfonylmethyl]azetidine-1-carboxylate (400 mg, 711 umol) in DCM (2 mL) was added TFA (3.08 g, 27.0 mmol, 2 mL). The mixture was stirred at 25° C. for 0.5 hrs. On completion, the mixture was filtered and concentrated in vacuo to give the title compound (328 mg, 99% yield) as yellow oil. LC-MS (ESI⁺) m/z 462.1 (M+H)⁺.

### Synthesis of 6-Chloro-8-isopropyl-2-[2-methyl-4-[[1-(4-piperidyl)azetidin-3-yl]methylsulfonyl]anilino] pyrido[2,3-d]pyrimidin-7-one (Intermediate KT)

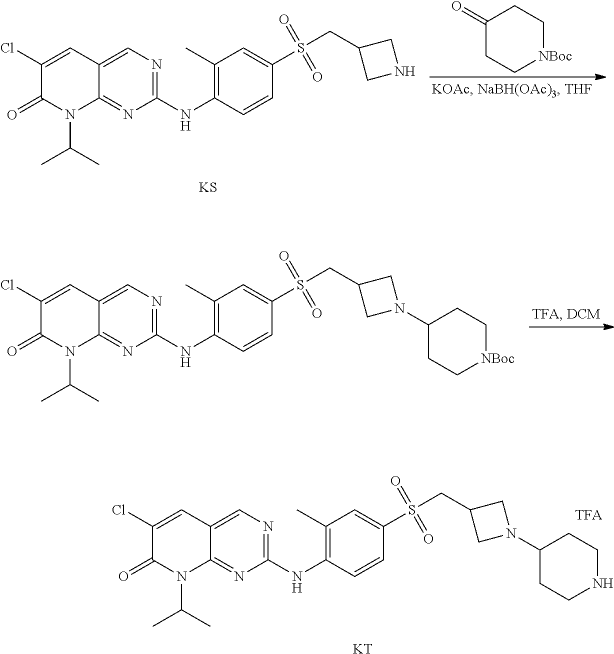 [SMILES: Cc1cc(S(=O)(=O)CC2CNC2)ccc1Nc1ncc2cc(Cl)c(=O)n(C(C)C)c2n1 ; CC(C)(C)OC(=O)N1CCC(=O)CC1 ; "KS" ; Cc1cc(S(=O)(=O)CC2CN(C3CCN(C(=O)OC(C)(C)C)CC3)C2)ccc1Nc1ncc2cc(Cl)c(=O)n(C(C)C)c2n1 ; Cc1cc(S(=O)(=O)CC2CN(C3CCNCC3)C2)ccc1Nc1ncc2cc(Cl)c(=O)n(C(C)C)c2n1 ; "KT"]

### Step 1—Tert-butyl 4-[3-[[4-[(6-chloro-8-isopropyl-7-oxo-pyrido[2,3-d]pyrimidin-2-yl)amino]-3-methyl-phenyl]sulfonylmethyl]azetidin-1-yl]piperidine-1-carboxylate

To a solution of tert-butyl 4-oxopiperidine-1-carboxylate (1.41 g, 7.10 mmol, CAS #79099-07-3) and 2-[4-(azetidin-3-ylmethylsulfonyl)-2-methyl-anilino]-6-chloro-8-isopropyl-pyrido[2,3-d]pyrimidin-7-one (328 mg, 710 umol, Intermediate KS) in THF (10 mL) was added dropwise KOAc (1.39 g, 14.2 mmol) at 0° C. After 30 minutes, the NaBH(OAc)₃ (1.50 g, 7.10 mmol) was added dropwise. The resulting mixture was stirred at 0° C. for 2 hrs. On completion, the mixture was concentrated in vacuo to give a residue. Then the residue was purified by prep-HPLC (column: Welch Xtimate C18 150*25 mm*5 um; mobile phase: [water (FA)-ACN]; B %: 12%-42%, 10 min) to give the title compound (400 mg, 85% yield) as a white solid. ¹H NMR (400 MHz, DMSO-d₆) δ 9.69 (s, 1H), 8.76 (s, 1H), 8.18 (s, 1H), 7.87 (d, J=8.0 Hz, 1H), 7.76 (s, 1H), 7.69 (dd, J=2.0, 8.4 Hz, 1H), 5.70-5.57 (m, 1H), 3.65 (d, J=3.6 Hz, 2H), 3.63 (d, J=4.4 Hz, 4H), 3.56 (d, J=7.6 Hz, 2H), 2.59-2.55 (m, 1H), 2.38 (s, 3H), 2.27-2.18 (m, 1H), 1.68 (d, J=4.0 Hz, 2H), 1.65 (d, J=4.0 Hz, 2H), 1.54-1.50 (m, 2H), 1.44 (d, J=6.8 Hz, 6H), 1.37 (s, 9H). LC-MS (ESI⁺) m/z 645.2 (M+H)⁺.

### Step 2—6-Chloro-8-isopropyl-2-[2-methyl-4-[[1-(4-piperidyl)azetidin-3-yl]methylsulfonyl]anilino]pyrido[2,3-d]pyrimidin-7-one

To a solution of tert-butyl 4-[3-[[4-[(6-chloro-8-isopropyl-7-oxo-pyrido[2,3-d]pyrimidin-2-yl)amino]-3-methyl-phenyl]sulfonylmethyl]azetidin-1-yl]piperidine-1-carboxylate (120 mg, 185 umol) in DCM (0.5 mL) was added TFA (0.5 mL). The mixture was stirred at 25° C. for 1 hr. On completion, the reaction mixture was filtered and concentrated in vacuo to give the title compound (100 mg, 98% yield, TFA salt) as yellow oil. LC-MS (ESI⁺) m/z 545.2 (M+H)⁺.

### Synthesis of N-isopropylacetamidine (Intermediate DW)

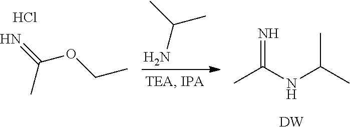

To a solution of ethyl ethanimidate hydrochloride (10.0 g, 80.9 mmol, CAS #2208-07-3) in IPA (60 mL) was added TEA (8.19 g, 80.9 mmol) and propan-2-amine (4.78 g, 80.9 mmol, CAS #4432-77-3). The mixture was stirred at 25° C. for 1 hour. On completion, the reaction mixture was concentrated in vacuo to give the title compound (5.5 g, 67% yield) as a colorless oil.

### Synthesis of 3-Chloro-4,4-diethoxy-butan-2-one (Intermediate DX)

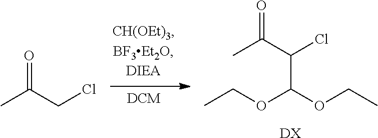

To a stirred solution of diethoxymethoxyethane (16.0 g, 108 mmol, CAS #122-51-0) in DCM (150 mL) was added diethyloxonio(trifluoro)boranuide (32.6 g, 108 mmol, 47% solution) at −30° C. under N₂ atmosphere. The reaction mixture was allowed to stir at 25° C. for 1 hr. Then 1-chloropropan-2-one (5.00 g, 54.0 mmol, CAS #78-95-5) was added rapidly at −78° C. followed by DIPEA (20.9 g, 162 mmol). Then the reaction mixture was allowed to stir at −78° C. for 1 hr. The reaction mass was added saturated NaHCO₃ (100 mL) and stirred for 15 mins and the layer was separated. The aqueous phase was extracted with DCM (2×100 mL). The combined organic layer was washed with H₂SO₄: H₂O (1:10) ratio followed by water (2×100 mL). The organic layer was dried over anhydrous Na₂SO₄ and concentrated under reduced pressure to give the title compound (10.0 g, 47% yield) as a red oil.

### Synthesis of [4-(3-Isopropyl-2-methyl-imidazol-4-yl)pyrimidin-2-yl] trifluoromethanesulfonate (Intermediate DY)

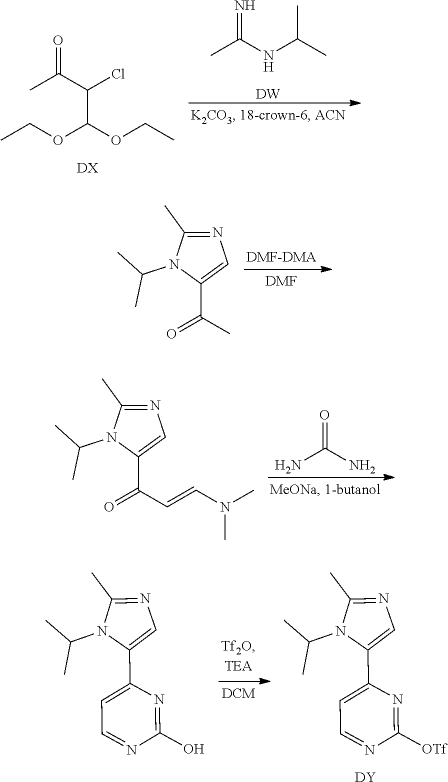

### Step 1—1-(3-Isopropyl-2-methyl-imidazol-4-yl)ethanone

A mixture of 3-chloro-4,4-diethoxy-butan-2-one (10.0 g, 51.3 mmol, Intermediate DX), N-isopropylacetamidine (5.15 g, 51.3 mmol, Intermediate DW), K₂CO₃ (21.3 g, 154 mmol) and 18-CROWN-6 (678 mg, 2.57 mmol) in ACN (100 mL) was degassed and purged with N₂ three times. Then the mixture was stirred at 80° C. for 16 hrs under N₂ atmosphere. The reaction mixture was partitioned between H₂O (100 mL) and EA (2×100 mL). The organic phase was separated, washed with brine (100 mL), dried over anhydrous Na₂SO₄, filtered and concentrated in vacuo to give a residue. The residue was purified by column chromatography (SiO₂, DCM/IPA=100/1 to 10/1) to give the title compound (5.00 g, 58% yield) as a red oil. ¹H NMR (400 MHz, CDCl₃) δ 7.72 (s, 1H), 5.43-5.22 (m, 1H), 2.54 (s, 3H), 2.46 (s, 3H), 1.52 (s, 3H), 1.50 (s, 3H). LCMS (ESI⁺) m/z 167.1 (M+H)⁺.

### Step 2—(E)-3-(Dimethylamino)-1-(3-isopropyl-2-methyl-imidazol-4-yl)prop-2-en-1-one

To a solution of 1-(3-isopropyl-2-methyl-imidazol-4-yl)ethanone (5.00 g, 30.0 mmol) in DMF (30 mL) was added DMF-DMA (3.94 g, 33.0 mmol, CAS #4637-24-5). The mixture was stirred at 130° C. for 16 hrs. The reaction mixture was concentrated in vacuo to remove solvent. The residue was purified by column chromatography (SiO₂, DCM/IPA=100/1 to 10/1) to give the title compound (3.00 g, 45% yield) as a red solid. ¹H NMR (400 MHz, CDCl₃) δ 7.66 (d, J=12.4 Hz, 1H), 7.48 (s, 1H), 5.49 (d, J=12.4 Hz, 1H), 5.47-5.40 (m, 1H), 3.14-2.87 (m, 6H), 2.60 (s, 3H), 1.56 (s, 3H), 1.54 (s, 3H). LCMS (ESI⁺) m/z 222.2 (M+H)⁺.

### Step 3-4-(3-Isopropyl-2-methyl-imidazol-4-yl)pyrimidin-2-ol

To a solution of (E)-3-(dimethylamino)-1-(3-isopropyl-2-methyl-imidazol-4-yl)prop-2-en-1-one (2.00 g, 9.04 mmol), CH₃ONa (1.95 g, 36.1 mmol) and urea (1.36 g, 22.5 mmol, CAS #506-89-8) in 1-butanol (20 mL) was stirred at 140° C. for 16 hrs. On completion, the reaction mixture was concentrated in vacuo to remove the solvent. The residue was purified by prep-HPLC (column: Phenomenex C18 250*50 mm*10 um; mobile phase: [water (ammonia hydroxide v/v)-ACN]; B %: 0%-20%, 8 min) to give the title compound (1.10 g, 55% yield) as a white solid. LCMS (ESI⁺) m/z 219.0 (M+H)⁺.

### Step 4—[4-(3-Isopropyl-2-methyl-imidazol-4-yl)pyrimidin-2-yl] trifluoromethanesulfonate

To a solution of 4-(3-isopropyl-2-methyl-imidazol-4-yl)pyrimidin-2-ol (550 mg, 2.52 mmol) in DCM (5 mL) was added TEA (509 mg, 5.04 mmol) and Tf₂O (746 mg, 2.65 mmol). The mixture was stirred at 0° C. for 1 hr. On completion, the reaction mixture was partitioned between H₂O (50 mL) and DCM (50 mL). The organic phase was separated, washed with brine (50 mL), dried over anhydrous Na₂SO₄, filtered and concentrated in vacuo to give a residue to give the title compound (780 mg, 88% yield) as a red solid. LCMS (ESI⁺) m/z 350.9 (M+H)⁺.

### Synthesis of 4-[[4-(3-Isopropyl-2-methyl-imidazol-4-yl)pyrimidin-2-yl]amino]-3-methyl-benzenesulfonyl chloride (Intermediate DZ)

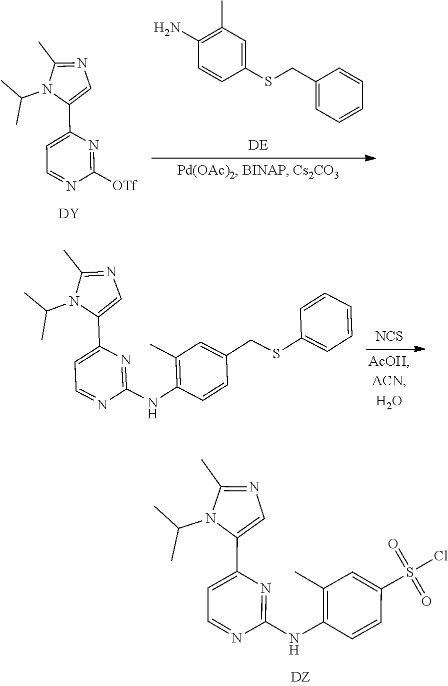

### Step 1—N-(4-Benzylsulfanyl-2-methyl-phenyl)-4-(3-isopropyl-2-methyl-imidazol-4-yl) pyrimidin-2-amine

A mixture of [4-(3-isopropyl-2-methyl-imidazol-4-yl)pyrimidin-2-yl] trifluoromethanesulfonate (780 mg, 2.23 mmol, Intermediate DY), 4-benzylsulfanyl-2-methyl-aniline (459 mg, 2.00 mmol, Intermediate DE), Pd(OAc)₂ (49.9 mg, 222 umol), BINAP (138 mg, 222 umol) and Cs₂CO₃ (2.18 g, 6.68 mmol) in toluene (10 mL) was degassed and purged with N₂ three times. Then the mixture was stirred at 100° C. for 16 hrs under N₂ atmosphere. The reaction mixture was filtered and concentrated in vacuo to give a residue. The residue was purified by prep-HPLC (column: Phenomenex Luna C18 200*40 mm*10 um; mobile phase: [water(FA)-ACN]; B %: 25%-55%, 10 min) to give the title compound (270 mg, 28% yield) as a yellow solid. LCMS (ESI⁺) m/z 430.4 (M+H)⁺.

### Step 2—4-[[4-(3-Isopropyl-2-methyl-imidazol-4-yl)pyrimidin-2-yl]amino]-3-methyl-benzenesulfonyl chloride

To a solution of N-(4-benzylsulfanyl-2-methyl-phenyl)-4-(3-isopropyl-2-methyl-imidazol-4-yl) pyrimidin-2-amine (30.0 mg, 69.8 umol) in a mixture solution of ACN (1 mL), AcOH (0.1 mL) and H₂O (0.02 mL) was added NCS (23.3 mg, 174 umol). The mixture was stirred under dark at 25° C. for 1 hrs. The mixture was diluted with water (15 mL), and extracted with EA (3×10 mL). The combined organic layer was washed with brine (30 mL), dried with anhydrous Na₂SO₄, filtered and concentrated in vacuo to give the title compound (28.0 mg, 98% yield) as a yellow solid. LCMS (ESI⁺) m/z 405.7 (M+H)⁺.

### Synthesis of 4-[3-[4-[[4-(3-Isopropyl-2-methyl-imidazol-4-yl)pyrimidin-2-yl]amino]-3-methyl-phenyl] sulfonylpropoxy]cyclohexanecarbaldehyde (Intermediate KU)

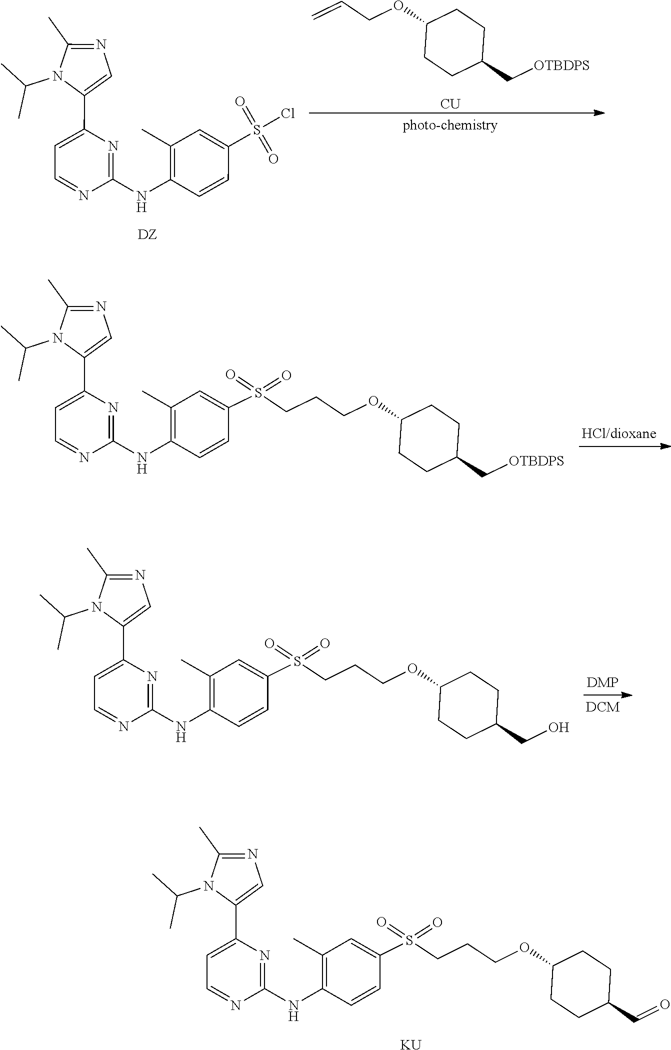

### Step 1—N-[4-[3-[4-[[tert-butyl(diphenyl)silyl]oxymethyl]cyclohexoxy]propylsulfonyl]-2-methyl-phenyl]-4-(3-isopropyl-2-methyl-imidazol-4-yl)pyrimidin-2-amine

An oven-dried 15 mL vial equipped with magnetic stir bar was charged with (4-allyloxycyclohexyl) methoxy-tert-butyl-diphenyl-silane (82.0 mg, 200 umol, Intermediate CU), 4-[[4-(3-isopropyl-2-methyl-imidazol-4-yl)pyrimidin-2-yl]amino]-3-methyl-benzenesulfonyl chloride (203 mg, 501 umol, Intermediate DZ), tris[2-(2-pyridyl)phenyl]iridium (656 ug, 1.00 umol), bis(trimethylsilyl)silyl-trimethyl-silane (99.7 mg, 401 umol) and 4-sulfanylphenol (5.06 mg, 40.1 umol) in ACN (3 mL). The vial was sealed and placed under nitrogen was added. The reaction was stirred and irradiated with a 10 W blue LED lamp (3 cm away), with cooling water to keep the reaction temperature at 25° C. for 14 hrs. The reaction mixture was filtered and concentrated in vacuo to give a residue. The residue was purified by prep-HPLC (column: Phenomenex luna C18 150*25 mm*10 um; mobile phase: [water(FA)-ACN]; B %: 51%-81%, 9 min) to give the title compound (40.0 mg, 26% yield) as a white solid. ¹H NMR (400 MHz, DMSO-d₆) δ 9.06 (s, 1H), 8.39 (d, J=5.2 Hz, 1H), 7.79 (d, J=8.4 Hz, 1H), 7.75 (s, 1H), 7.68 (dd, J=1.6, 8.0 Hz, 1H), 7.58 (dd, J=1.6, 7.2 Hz, 4H), 7.46-7.40 (m, 7H), 7.10 (d, J=5.2 Hz, 1H), 5.53-5.44 (m, 1H), 3.44-3.41 (m, 4H), 3.25 (d, J=7.6 Hz, 2H), 2.43 (s, 3H), 2.34 (s, 3H), 1.94-1.87 (m, 2H), 1.72 (d, J=9.6 Hz, 3H), 1.48-1.38 (m, 2H), 1.25 (d, J=7.2 Hz, 6H), 1.10-1.02 (m, 3H), 0.98 (s, 9H), 0.93 (d, J=13.2 Hz, 2H). LCMS (ESI⁺) m z 780.4 (M+H)⁺.

### Step 2—[4-[3-[4-[[4-(3-Isopropyl-2-methyl-imidazol-4-yl)pyrimidin-2-yl]amino]-3-methyl-phenyl]sulfonylpropoxy]cyclohexyl]methanol

To a solution of N-[4-[3-[4-[[tert-butyl(diphenyl)silyl]oxymethyl]cyclohexoxy]propylsulfonyl]-2-methyl-phenyl]-4-(3-isopropyl-2-methyl-imidazol-4-yl)pyrimidin-2-amine (30.0 mg, 38.4 umol) in DCM (1 mL) was added HCl/dioxane (4 M, 1 mL). The mixture was stirred at 25° C. for 3 hrs. On completion, the reaction mixture was concentrated in vacuo to give a residue. The residue was purified by prep-HPLC (column: Phenomenex luna C18 150*25 mm*10 um; mobile phase: [water(FA)-ACN]; B %: 9%-39%, 9 min) to give the title compound (20.0 mg, 96% yield) as a white solid. LCMS (ESI⁺) m/z 542.2 (M+H)⁺.

### Step 3—4-[3-[4-[[4-(3-Isopropyl-2-methyl-imidazol-4-yl)pyrimidin-2-yl]amino]-3-methyl-phenyl]sulfonylpropoxy]cyclohexanecarbaldehyde

To a solution of [4-[3-[4-[[4-(3-isopropyl-2-methyl-imidazol-4-yl)pyrimidin-2-yl]amino]-3-methyl-phenyl]sulfonylpropoxy]cyclohexyl]methanol (24.0 mg, 44.3 umol) in DCM (2 mL) was added DMP (22.5 mg, 53.1 umol). The mixture was stirred at 25° C. for 1 hr. The reaction mixture was quenched with Na₂S₂O₃·5H₂O (10 mL), and extracted with DCM (3×20 mL). The combined organic layer was washed with brine (20 mL), dried over anhydrous sodium sulfate, filtered and the filtrate was concentrated in vacuo to give the title compound (23.0 mg, 96% yield) as a white solid. LCMS (ESI⁺) m/z 540.3 (M+H)⁺.

### Synthesis of 4-[3-[3-Methyl-4-[[4-(1-methylpyrazol-4-yl)-5-(trifluoromethyl)pyrimidin-2-yl]amino]phenyl]sulfonylpropoxy]cyclohexanecarbaldehyde (Intermediate KV)

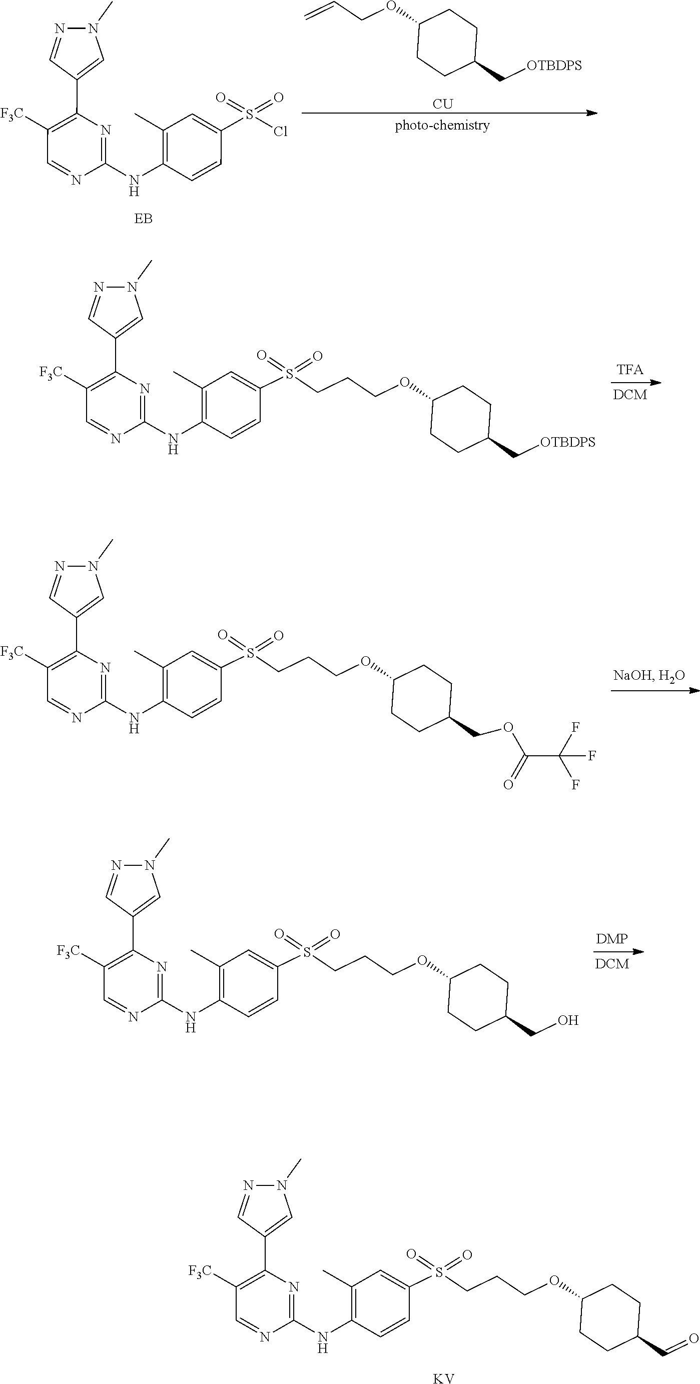

### Step 1—N-[4-[3-[4-[[tert-butyl(diphenyl)silyl]oxymethyl]cyclohexoxy]propylsulfonyl]-2-methyl-phenyl]-4-(1-methylpyrazol-4-yl)-5-(trifluoromethyl)pyrimidin-2-amine

To a solution of (4-allyloxycyclohexyl)methoxy-tert-butyl-diphenyl-silane (150 mg, 367 umol, Intermediate CU) and 3-methyl-4-[[4-(1-methylpyrazol-4-yl)-5-(trifluoromethyl)pyrimidin-2-yl]amino]benzenesulfonyl chloride (396 mg, 917 umol, Intermediate EB) in ACN (3 mL) was added 4-bromopyridine (11.6 mg, 73.4 umol), Ir[dF(CF₃)ppy]2(dtbpy)(PF₆) (4.12 mg, 3.67 umol), TTMSS (182 mg, 734 umol, 226 uL) and IR(PPY)₃ (1.20 mg, 1.84 umol). The mixture was stirred at 25° C. for 14 hrs. On completion, the mixture was filtered and concentrated in vacuo to give a residue. The residue was purified by prep-HPLC (column: Phenomenex Luna C18 200*40 mm*10 um; mobile phase: [water (TFA) -ACN]; B %: 80%-100%, 10 min) to give the title compound (120 mg, 41% yield, TFA salt) as a white solid. ¹H NMR (400 MHz, CDCl₃) δ 8.68 (s, 1H), 8.49 (d, J=8.8 Hz, 1H), 8.16 (s, 1H), 8.04 (s, 1H), 7.82 (d, J=8.8 Hz, 1H), 7.78 (s, 1H), 7.65 (d, J=6.4 Hz, 4H), 7.54 (s, 1H), 7.43-7.36 (m, 5H), 4.01 (s, 3H), 3.54 (t, J=6.0 Hz, 2H), 3.45 (d, J=6.0 Hz, 2H), 3.27-3.19 (m, 2H), 3.16-3.06 (m, 1H), 2.47 (s, 3H), 2.23-2.04 (m, 2H), 2.01-1.96 (m, 3H), 1.82 (d, J=12.4 Hz, 2H), 1.53-1.42 (m, 1H), 1.20-1.11 (m, 2H), 1.05 (s, 9H), 1.02-0.92 (m, 2H). LC-MS (ESI⁺) m/z 806.5 (M+H)⁺.

### Step 2—[4-[3-[3-Methyl-4-[[6-(1-methylpyrazol-4-yl)-5-(trifluoromethyl)-2-pyridyl]amino]phenyl]sulfonylpropoxy]cyclohexyl]methyl 2,2,2-trifluoroacetate

To a solution of N-[4-[3-[4-[[tert-butyl(diphenyl)silyl]oxymethyl]cyclohexoxy]propylsulfonyl]-2-methyl-phenyl]-4-(1-methylpyrazol-4-yl)-5-(trifluoromethyl)pyrimidin-2-amine (120 mg, 148 umol) in DCM (0.5 mL) was added TFA (770 mg, 6.75 mmol, 0.5 mL). The mixture was stirred at 25° C. for 2 hrs. On completion, the mixture was filtered and concentrated in vacuo to give a residue. The residue was purified by prep-HPLC (column: Welch Xtimate C18 150*25 mm*5 um; mobile phase: [water (TFA)-ACN]; B %: 52%-72%, 10 min) to give the title compound (70 mg, 61% yield, TFA salt) as a white solid. ¹H NMR (400 MHz, CDCl3) δ 8.66 (s, 1H), 8.38 (d, J=8.4 Hz, 1H), 8.15 (s, 1H), 8.11 (s, 1H), 8.04 (s, 1H), 7.84-7.78 (m, 2H), 4.16 (d, J=6.4 Hz, 2H), 4.02 (s, 3H), 3.55 (t, J=6.0 Hz, 2H), 3.26-3.20 (m, 2H), 3.19-3.11 (m, 1H), 2.47 (s, 3H), 2.07-1.97 (m, 4H), 1.86-1.78 (m, 2H), 1.78-1.67 (m, 1H), 1.27-1.15 (m, 2H), 1.10-0.99 (m, 2H). LC-MS (ESI⁺) m/z 664.1 (M+H)⁺.

### Step 3—[4-[3-[3-Methyl-4-[[4-(1-methylpyrazol-4-yl)-5-(trifluoromethyl)pyrimidin-2-yl]amino]phenyl]sulfonylpropoxy]cyclohexyl]methanol

To a solution of [4-[3-[3-methyl-4-[[4-(1-methylpyrazol-4-yl)-5-(trifluoromethyl)pyrimidin-2-yl]amino]phenyl]sulfonylpropoxy]cyclohexyl]methyl 2,2,2-trifluoroacetate (70.0 mg, 105 umol) in THF (1 mL) was added NaOH (4.22 mg, 105 umol) and H₂O (1.90 mg, 105 umol, 1.90 uL). The mixture was stirred at 25° C. for 10 mins. On completion, the mixture was concentrated in vacuo to give the title compound (59.0 mg, 98% yield) as a yellow solid. LC-MS (ESI⁺) m/z 568.1 (M+H)⁺.

### Step 4—4-[3-[3-Methyl-4-[[4-(1-methylpyrazol-4-yl)-5-(trifluoromethyl)pyrimidin-2-yl]amino]phenyl]sulfonylpropoxy]cyclohexanecarbaldehyde

To a solution of [4-[3-[3-methyl-4-[[4-(1-methylpyrazol-4-yl)-5-(trifluoromethyl)pyrimidin-2-yl]amino]phenyl]sulfonylpropoxy]cyclohexyl]methanol (59.0 mg, 103 umol) in DCM (1 mL) was added DMP (66.1 mg, 155 umol). The mixture was then stirred at 25° C. for 2 hrs. On completion, the reaction mixture was quenched with Na₂S₂O₃ (0.5 mL) at 25° C., and then diluted with NaHCO₃ (8 mL) and extracted with DCM (3×8 mL). The combined organic layers were washed with brine (2×5 mL), dried over anhydrous Na₂SO₄, filtered and concentrated in vacuo to give the title compound (50.0 mg, 86% yield) as yellow oil. LC-MS (ESI⁺) m/z 566.2 (M+H)⁺.

### Synthesis of 3-(4-Bromo-3-methyl-2-oxo-2,3-dihydro-1H-benzo[d]imidazol-1-yl)piperidine-2,6-dione (Intermediate DC)

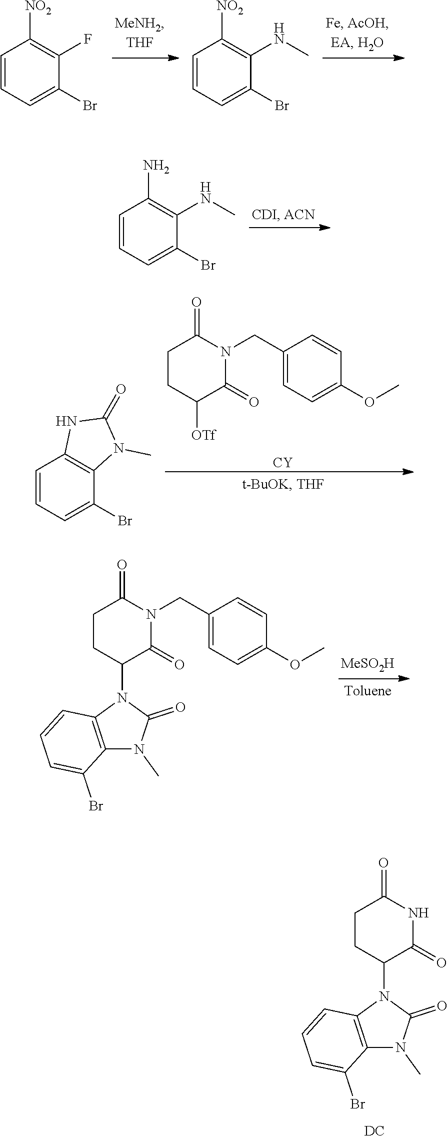

### Step 1—2-Bromo-N-methyl-6-nitro-aniline

To a solution of 1-bromo-2-fluoro-3-nitro-benzene (40.0 g, 181 mmol, CAS #58534-94-4) in THF (40 mL) was added MeNH₂ (2 M, 400 mL). The reaction mixture was stirred at 60° C. for 12 hours. On completion, the reaction mixture was poured into sat.NaHCO₃ (30 mL) and extracted with EA (3×200 mL). The combined organic layers were washed with brine (2×200 mL), dried with anhydrous Na₂SO₄, filtered and concentrated in vacuo to give the title compound (40.0 g, 95% yield) as red oil. LC-MS (ESI⁺) m/z 230.9 (M+H)⁺.

### Step 2—3-Bromo-N2-methyl-benzene-1,2-diamine

To a mixture of 2-bromo-N-methyl-6-nitro-aniline (23.0 g, 99.5 mmol) in EA (300 mL) and H₂O (10 mL) was added AcOH (100 mL). The mixture was warmed to 50° C. Then Fe (22.2 g, 398 mmol) was added to the reaction mixture and the mixture was heated to 80° C. about 4 hours. On completion, the reaction mixture was filtered and concentrated in vacuo. The residue was diluted with water (100 mL) and extracted with EA (3×200 mL). The combined organic layers was dried over Na₂SO₄, filtered and concentrated in vacuo to give the title compound (20.0 g, 99% yield) as red oil. ¹H NMR (400 MHz, DMSO-d₆) δ 6.73-6.70 (m, 1H), 6.68-6.60 (m, 2H), 5.02 (s, 2H), 3.67 (s, 1H), 2.58 (s, 3H).

### Step 3—4-Bromo-3-methyl-1H-benzimidazol-2-one

To a mixture of 3-bromo-N2-methyl-benzene-1,2-diamine (20.0 g, 99.4 mmol) in ACN (300 mL) was added CDI (32.2 g, 198 mmol). The reaction mixture was stirred at 85° C. for 12 hours under N₂ atmosphere. On completion, the reaction mixture was concentrated in vacuo. The reaction mixture was diluted with water (200 mL), where a solid precipitate was formed, which was filtered off. The solid was washed with water (1 L) and dried in vacuo to give the title compound (20.0 g, 88% yield) as white solid. ¹H NMR (400 MHz, DMSO-d₆) δ 11.17 (s, 1H), 7.14 (dd, J=1.2, 8.0 Hz, 1H), 7.00-6.95 (m, 1H), 6.93-6.87 (m, 1H), 3.55 (s, 3H).

### Step 4—3-(4-Bromo-3-methyl-2-oxo-benzimidazol-1-yl)-1-[(4-methoxyphenyl)methyl]piperidine-2,6-dione

To a solution of 4-bromo-3-methyl-1H-benzimidazol-2-one (12.0 g, 52.8 mmol) in THF (300 mL) was added t-BuOK (7.12 g, 63.4 mmol). The reaction mixture was stirred at 0° C. for 0.5 hr. Subsequently, [1-[(4-methoxyphenyl)methyl]-2,6-dioxo-3-piperidyl]trifluoromethanesulfonate (20.1 g, 52.8 mmol, Intermediate CY) in a solution of THF (100 mL) was added dropwise. The resulting reaction mixture was stirred at 20° C. for 0.5 hr under N₂. On completion, the reaction mixture was quenched with saturated NH₄Cl (100 mL), and extracted with ethyl acetate (200 mL). The combined organic layers were washed with brine (2×100 mL), dried over anhydrous sodium sulfate, filtered, the filtrate was concentrated in vacuo. The crude product was purified by reversed-phase HPLC (0.1% FA condition) to give the title compound (13.3 g, 55% yield) as a yellow solid. ¹H NMR (400 MHz, CDCl₃) δ 7.38 (d, J=8.8 Hz, 2H), 7.22 (d, J=8.0 Hz, 1H), 6.84 (d, J=8.8 Hz, 2H), 6.80 (t, J=8.0 Hz, 1H), 6.48-6.40 (d, J=8.0 Hz, 1H), 5.22 (dd, J=5.2, 12.8 Hz, 1H), 5.04-4.93 (m, 2H), 3.81 (s, 3H), 3.80 (s, 3H), 3.12-2.98 (m, 1H), 2.93-2.77 (m, 1H), 2.62 (dq, J=4.4, 13.2 Hz, 1H), 2.20-2.17 (m, 1H).

### Step 5—3-(4-bromo-3-methyl-2-oxo-2,3-dihydro-1H-benzo[d]imidazol-1-yl)piperidine-2,6-dione

A mixture of 3-(4-bromo-3-methyl-2-oxo-benzimidazol-1-yl)-1-[(4-methoxyphenyl)methyl]piperidine-2,6-dione (13.3 g, 29.0 mmol) in a mixed solvent of Tol. (80 mL) and methane sulfonic acid (40 mL) was degassed and purged with N₂ for 3 times, and then the mixture was stirred at 120° C. for 2 hrs under N₂ atmosphere. On completion, the reaction mixture was concentrated in vacuo to remove toluene. The residue was added 200 mL of ice water, and then white solid precipitate formed. The mixture was filtered and the filtered cake was collected and dried over in vacuo to give the title compound (7.30 g, 74% yield) as white solid. ¹H NMR (400 MHz, DMSO-d₆) δ 11.13 (s, 1H), 7.25 (d, J=8.0 Hz, 1H), 7.17 (d, J=8.0 Hz, 1H), 7.05-6.93 (m, 1H), 5.41 (dd, J=5.2, 12.8 Hz, 1H), 3.64 (s, 3H), 2.96-2.83 (m, 1H), 2.78-2.59 (m, 2H), 2.08-2.00 (m, 1H).

### Synthesis of 1-[1-[1-[(4-methoxyphenyl)methyl]-2,6-dioxo-3-piperidyl]-3-methyl-2-oxo-benzimidazol-4-yl]piperidine-4-carbaldehyde (Intermediate KW)

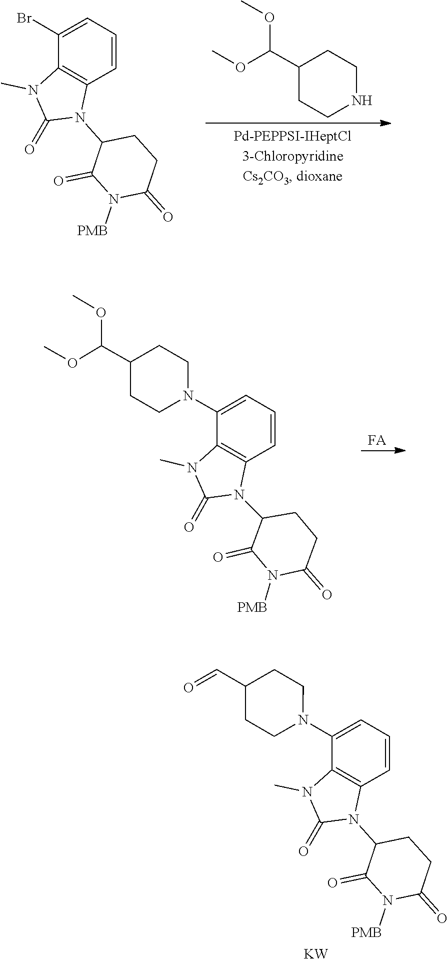

### Step 1—3-[4-[4-(dimethoxymethyl)-1-piperidyl]-3-methyl-2-oxo-benzimidazol-1-yl]-1-[(4-methoxyphenyl)methyl] piperidine-2, 6-dione

A mixture of 4-(dimethoxymethyl)piperidine (100 mg, 628 umol, CAS #188646-83-5), 3-(4-bromo-3-methyl-2-oxo-benzimidazol-1-yl)-1-[(4-methoxyphenyl)methyl]piperidine-2,6-dione (230 mg, 502 umol, synthesized via Steps 1-4 of Intermediate DC), Cs₂CO₃ (818 mg, 2.51 mmol) and Pd-PEPPSI-IHeptCl 3-Chloropyridine (54.0 mg, 62.8 umol) in dioxane (2 mL) was degassed and purged with N₂ three times. Then the mixture was stirred at 100° C. for 16 hrs under N₂ atmosphere. On completion, the mixture was filtered and concentrated in vacuo to give the title compound (50 mg, 15% yield) as white solid. LC-MS (ESI⁺) m/z 537.2 (M+H)⁺.

### Step 2—1-[1-[1-[(4-Methoxyphenyl)methyl]-2,6-dioxo-3-piperidyl]-3-methyl-2-oxo-benzimidazol-4-yl]piperidine-4-carbaldehyde

A mixture of 3-[4-[4-(dimethoxymethyl)-1-piperidyl]-3-methyl-2-oxo-benzimidazol-1-yl]-1-[(4-methoxyphenyl)methyl]piperidine-2,6-dione (50 mg, 93.1 umol) in HCOOH (1 mL) was stirred at 70° C. for 1 hr. On completion, the reaction mixture was concentrated under reduced pressure to give the title compound (45 mg, 98.45% yield) as yellow solid. LC-MS (ESI⁺) m/z 491.2 (M+H)⁺.

### Synthesis of 6-Chloro-8-isopropyl-2-methylsulfonyl-pyrido [2,3-d] pyrimidin-7-one (Intermediate KP)

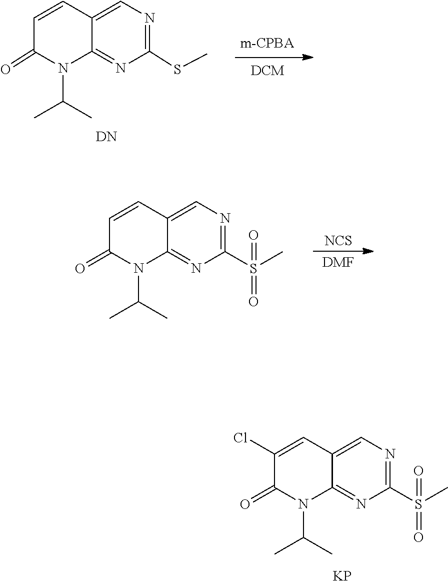

### Step 1—8-Isopropyl-2-methylsulfonyl-pyrido[2,3-d]pyrimidin-7-one

To a solution of 8-isopropyl-2-methylsulfanyl-pyrido[2,3-d]pyrimidin-7-one (2.20 g, 9.35 mmol, Intermediate DN) in DCM (20.0 mL) was added m-CPBA (7.59 g, 37.0 mmol, 85% solution). The mixture was stirred at 40° C. for 3 hrs. On completion, the reaction mixture was quenched with Na₂CO₃ aq. (100 mL) at 25° C., and then extracted with EA (3×100 mL). The combined organic layers were washed with brine (2×100 mL), dried over anhydrous Na₂SO₄, filtered and concentrated in vacuo to give the title compound (2.10 g, 84% yield) as a yellow solid. ¹H NMR (400 MHz, DMSO-d₆) δ 9.27 (s, 1H), 8.07 (d, J=9.6 Hz, 1H), 6.87 (d, J=9.6 Hz, 1H), 5.65 (td, J=6.8, 13.6 Hz, 1H), 3.46 (s, 3H), 1.56 (d, J=7.2 Hz, 6H). LC-MS (ESI⁺) m/z 267.9 (M+H)⁺.

### Step 2—6-Chloro-8-isopropyl-2-methylsulfonyl-pyrido[2,3-d]pyrimidin-7-one

To a solution of 8-isopropyl-2-methylsulfonyl-pyrido[2,3-d]pyrimidin-7-one (100 mg, 374 umol) in DMF (1.50 mL) was added NCS (149 mg, 1.12 mmol). The mixture was stirred at 70° C. for 16 hrs. On completion, the mixture was concentrated in vacuo. The mixture was purified by reversed phase (0.1% FA) to give the title compound (74.0 mg, 65% yield) as a yellow solid. ¹H NMR (400 MHz, DMSO-d₆) δ 9.27-9.25 (m, 1H), 8.68-8.37 (m, 1H), 5.90-5.58 (m, 1H), 3.48 (d, J=2.4 Hz, 3H), 1.58 (s, 6H). LC-MS (ESI⁺) m/z 301.8 (M+H)⁺.

### Synthesis of 6-chloro-8-isopropyl-2-[2-methyl-4-(4-piperidylsulfonyl)anilino]pyrido[2,3-d]pyrimidin-7-one (Intermediate KX)

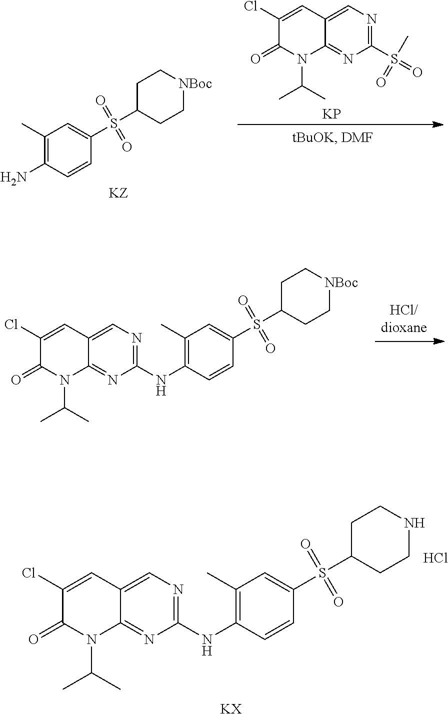

### Step 1—Tert-butyl 4-[4-[(6-chloro-8-isopropyl-7-oxo-pyrido[2,3-d]pyrimidin-2-yl)amino]-3-methyl-phenyl]sulfonylpiperidine-1-carboxylate

To a solution of tert-butyl 4-(4-amino-3-methyl-phenyl)sulfonylpiperidine-1-carboxylate (250 mg, 705 umol, Intermediate KZ) in DMF (2 mL) was added t-BuOK (395 mg, 3.53 mmol) at 0° C., then the 6-chloro-8-isopropyl-2-methylsulfonyl-pyrido[2,3-d]pyrimidin-7-one (170 mg, 564 umol, Intermediate KP) was added. The reaction mixture was stirred at 25° C. for 1 hr. On completion, the residue was diluted with water (10 mL), then the residue was extracted with EA (3×20 mL). The combined organic layer was dried over Na₂SO₄, filtered and filtrate was concentrated in vacuo. The residue was purified by column chromatography to give the title compound (300 mg, 73% yield) as yellow solid. ¹H NMR (400 MHz, DMSO-d₆) δ 9.72 (s, 1H), 8.76 (s, 1H), 8.17 (s, 1H), 7.86-7.82 (m, 1H), 7.75-7.73 (m, 1H), 7.67 (dd, J=1.6, 8.4 Hz, 1H), 5.63-5.53 (m, 1H), 4.04-3.98 (m, 2H), 3.49-3.40 (m, 1H), 2.88 (s, 3H), 2.73 (s, 3H), 2.36 (s, 3H), 1.89-1.84 (m, 2H), 1.36 (s, 15H). LC-MS (ESI⁺) m/z 576.0 (M+H)⁺.

### Step 2—6-Chloro-8-isopropyl-2-[2-methyl-4-(4-piperidylsulfonyl)anilino]pyrido[2,3-d]pyrimidin-7-one

A solution of tert-butyl 4-[4-[(6-chloro-8-isopropyl-7-oxo-pyrido[2,3-d]pyrimidin-2-yl)amino]-3-methyl-phenyl]sulfonylpiperidine-1-carboxylate (100 mg, 173 umol) in HCl/dioxane (3 mL) was stirred at 25° C. for 0.5 hr. On completion, the reaction mixture was concentrated in vacuo to give the title compound (88.0 mg, 98% yield, HCl) as yellow oil. LC-MS (ESI⁺) m/z 476.2 (M+H)⁺.

### Synthesis of 6-chloro-8-isopropyl-2-[2-methyl-4-[[1-(4-piperidyl)-4-piperidyl]sulfonyl]anilino]pyrido[2,3-d]pyrimidin-7-one (Intermediate KY)

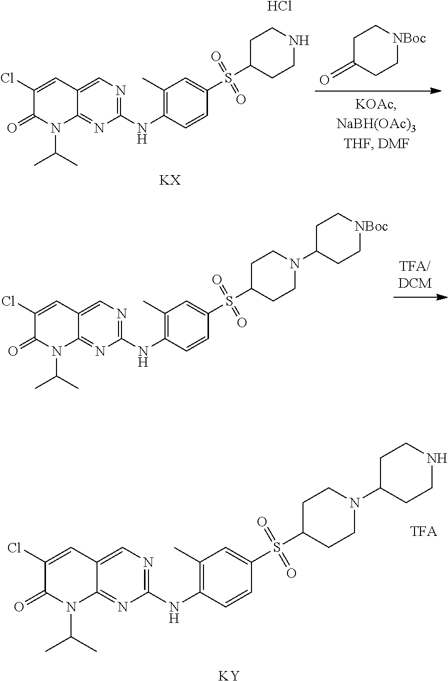

### Step 1—Tert-butyl 4-[4-[4-[(6-chloro-8-isopropyl-7-oxo-pyrido[2,3-d]pyrimidin-2-yl)amino]-3-methyl-phenyl]sulfonyl-1-piperidyl]piperidine-1-carboxylate

To a solution of 6-chloro-8-isopropyl-2-[2-methyl-4-(4-piperidylsulfonyl)anilino]pyrido[2,3-d]pyrimidin-7-one (170 mg, 332 umol, HCl salt, Intermediate KX) in THF (2 mL) was added TEA (67.1 mg, 663 umol). Then tert-butyl 4-oxopiperidine-1-carboxylate (330 mg, 1.66 mmol, CAS #79099-07-3) and HOAc (19.9 mg, 331 umol) were added, and the mixture was stirred at 25° C. for 0.5 hrs. Next, NaBH(OAc)₃ (140 mg, 663 umol) was added and the mixture was stirred at 25° C. for 12 hrs. On completion, the mixture was quenched with water (1 mL), filtered and the filtrate was concentrated in vacuo to give the residue. The residue was purified by prep-HPLC (column: Phenomenex luna C18 150*25 mm*10 um; mobile phase: [water(FA)-ACN]; B %: 20%-50%, 8 min) to give the title compound (120 mg, 55% yield) as white solid. ¹H NMR (400 MHz, DMSO-d₆) δ 9.72 (s, 1H), 8.76 (s, 1H), 8.17 (s, 1H), 7.85-7.78 (m, 1H), 7.72 (d, J=1.6 Hz, 1H), 7.66 (dd, J=2.0, 8.4 Hz, 1H), 5.65-5.51 (m, 1H), 3.95-3.88 (m, 2H), 3.63-3.59 (m, 2H), 2.92-2.87 (m, 2H), 2.35 (s, 3H), 2.12 (brt, J=10.6 Hz, 2H), 1.87-1.81 (m, 2H), 1.69-1.57 (m, 4H), 1.38 (s, 6H), 1.37 (s, 9H), 1.29-1.14 (m, 4H); LC-MS (ESI⁺) m/z 659.2 (M+H)⁺.

### Step 2—6-Chloro-8-isopropyl-2-[2-methyl-4-[[1-(4-piperidyl)-4-piperidyl]sulfonyl]anilino]pyrido[2,3-d]pyrimidin-7-one

To a solution of tert-butyl 4-[4-[4-[(6-chloro-8-isopropyl-7-oxo-pyrido[2,3-d]pyrimidin-2-yl)amino]-3-methyl-phenyl]sulfonyl-1-piperidyl]piperidine-1-carboxylate (60 mg, 91.0 umol) in DCM (1.0 mL) was added TFA (770 mg, 6.75 mmol), then the mixture was stirred at 25° C. for 1 hr. On completion, the mixture was concentrated in vacuo to give the title compound (60 mg, 98% yield, TFA) as yellow oil. LC-MS (ESI⁺) m/z 559.2 (M+H)⁺.

### Synthesis of Tert-butyl 4-(4-amino-3-methyl-phenyl) sulfonylpiperidine-1-carboxylate (Intermediate KZ)

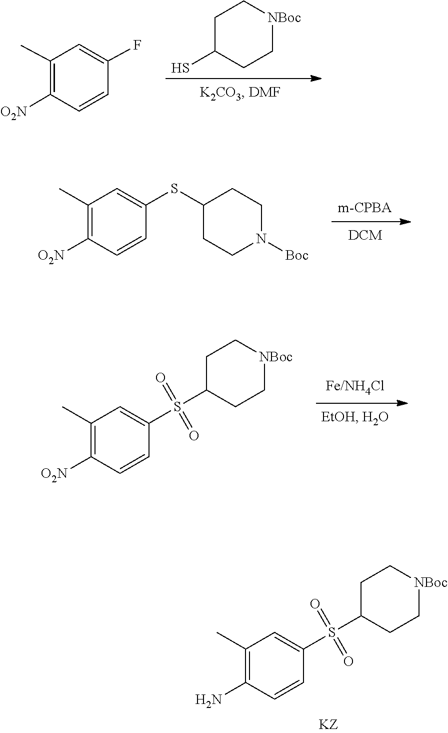 [SMILES: Cc1cc(F)ccc1[N+](=O)[O-] ; CC(C)(C)OC(=O)N1CCC(S)CC1 ; Cc1cc(SC2CCN(C(=O)OC(C)(C)C)CC2)ccc1[N+](=O)[O-] ; Cc1cc(S(=O)(=O)C2CCN(C(=O)OC(C)(C)C)CC2)ccc1[N+](=O)[O-] ; Cc1cc(S(=O)(=O)C2CCN(C(=O)OC(C)(C)C)CC2)ccc1N]

### Step 1—Tert-butyl 4-(3-methyl-4-nitro-phenyl)sulfanylpiperidine-1-carboxylate

To a solution of 4-fluoro-2-methyl-1-nitro-benzene (2.14 g, 13.8 mmol, CAS #446-33-3) and tert-butyl 4-sulfanylpiperidine-1-carboxylate (2.50 g, 11.5 mmol, CAS #134464-79-2) in DMF (30 mL) was added K₂CO₃ (3.18 g, 23.0 mmol), then the mixture was stirred at 25° C. for 8 hrs. On completion, the mixture was diluted with water (30 mL) and extracted with EA (20 mL×3). The combined organic layer was dried over anhydrous Na₂SO₄, filtered and concentrated in vacuo to give the residue. The residue was purified by column chromatography (SiO₂, PE:EA=15:1 to 7:1) to give the title compound (3.60 g, 88% yield) as yellow solid. ¹H NMR (400 MHz, DMSO-d₆) δ 7.96 (d, J=8.8 Hz, 1H), 7.44 (d, J=1.6 Hz, 1H), 7.39-7.38 (m, 1H), 3.83 (d, J=13.6 Hz, 2H), 3.78-3.70 (m, 1H), 3.09-2.92 (m, 2H), 2.52 (s, 3H), 2.02-1.90 (m, 2H), 1.46-1.40 (m, 2H), 1.39 (s, 9H).

### Step 2—Tert-butyl 4-(3-methyl-4-nitro-phenyl)sulfonylpiperidine-1-carboxylate

To a solution of tert-butyl 4-(3-methyl-4-nitro-phenyl)sulfanylpiperidine-1-carboxylate (1.00 g, 2.84 mmol) in DCM (10 mL) was added MCPBA (2.45 g, 14.1 mmol) at 0° C., then the mixture was stirred at 25° C. for 1 hr. On completion, the mixture was quenched with Na₂SO₃ (10 mL) and Na₂CO₃ (8 mL) at 0° C., diluted with water (8 mL) and extracted with DCM (8 mL×3). The combined organic layer was dried over anhydrous Na₂SO₄, filtered and concentrated in vacuo to give the residue. The residue was purified by column chromatography (SiO₂, PE:EA=4:1 to 1:1) to give the title compound (900 mg, 82% yield) as yellow solid. ¹H NMR (400 MHz, DMSO-d₆) δ 8.20 (d, J=8.4 Hz, 1H), 8.01 (d, J=1.2 Hz, 1H), 7.90-7.89 (m, 1H), 4.01 (d, J=11.6 Hz, 2H), 3.73-3.54 (m, 1H), 2.75-2.64 (m, 2H), 2.58 (s, 3H), 1.84 (d, J=11.6 Hz, 2H), 1.45-1.38 (m, 2H), 1.37 (s, 9H).

### Step 3—Tert-butyl 4-(4-amino-3-methyl-phenyl) sulfonylpiperidine-1-carboxylate

To a solution of tert-butyl 4-(3-methyl-4-nitro-phenyl)sulfonylpiperidine-1-carboxylate (0.400 g, 1.04 mmol) in EtOH (10 mL) and H₂O (2 mL) was added Fe (348 mg, 6.24 mmol) and NH₄Cl (556 mg, 10.4 mmol). The reaction mixture was stirred at 80° C. for 2 hrs. On completion, the reaction mixture was filtered and filtrate was concentrated in vacuo. The residue was diluted with water (10 mL), then extracted with EA (3×20 mL). The combined organic layer was dried over Na₂SO₄, filtered and filtrate was concentrated in vacuo. The residue was purified by column chromatography to give the title compound (300 mg, 81% yield) as yellow solid. LC-MS (ESI⁺) m/z 298.9 (M−56)⁺.

### Synthesis of 2-[1-[1-(2,6-dioxo-3-piperidyl)-3-methyl-2-oxo-benzimidazol-4-yl]-4-piperidyl]acetaldehyde (Intermediate LA)

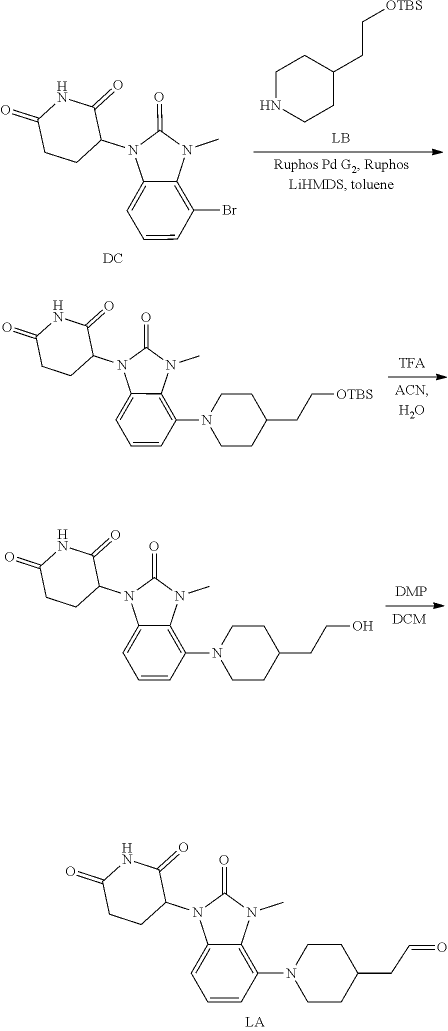 [SMILES: Cn1c(=O)n(C2CCC(=O)NC2=O)c2cccc(Br)c21 ; CC(C)(C)[Si](C)(C)OCCC1CCNCC1 ; "DC" ; Cn1c(=O)n(C2CCC(=O)NC2=O)c2cccc(N3CCC(CCO[Si](C)(C)C(C)(C)C)CC3)c21 ; Cn1c(=O)n(C2CCC(=O)NC2=O)c2cccc(N3CCC(CCO)CC3)c21 ; Cn1c(=O)n(C2CCC(=O)NC2=O)c2cccc(N3CCC(CC=O)CC3)c21]

### Step 1—3-[4-[4-[2-[Tert-butyl(dimethyl)silyl]oxyethyl]-1-piperidyl]-3-methyl-2-oxo-benzimidazol-1-yl]piperidine-2,6-dione

To a solution of tert-butyl-dimethyl-[2-(4-piperidyl)ethoxy]silane (863 mg, 3.55 mmol, Intermediate LB), 3-(4-bromo-3-methyl-2-oxobenzimidazol-1-yl)piperidine-2,6-dione (600 mg, 1.77 mmol, Intermediate DC) in toluene (10.0 mL) was added [2-(2-aminophenyl)phenyl]-chloro-palladium; dicyclohexyl-[2-(2,6-diisopropoxyphenyl)phenyl]phosphane (137 mg, 177 umol), RuPhos (82.8 mg, 177 umol) and LiHMDS (1.00 M, 8.87 mL) under N₂. The mixture was stirred at 80° C. for 1 hr under N₂. On completion, the mixture was concentrated in vacuo. The residue was then diluted with DMF (6.00 mL), filtered and the filtrate was acidified with FA until pH=5. The filtrate was concentrated in vacuo. The mixture was purified by reverse phase: (0.1% FA) to give the title compound (460 mg, 51% yield) as yellow solid. ¹H NMR (400 MHz, DMSO-d₆) δ 11.08 (s, 1H), 7.02-6.78 (m, 3H), 5.40-5.30 (m, 1H), 3.67 (t, J=6.4 Hz, 2H), 3.61 (s, 3H), 3.15-3.05 (m, 2H), 2.97-2.81 (m, 1H), 2.74-2.66 (m, 2H), 2.65-2.56 (m, 2H), 2.04-1.93 (m, 1H), 1.80-1.70 (m, 2H), 1.55-1.45 (m, 3H), 1.44-1.31 (m, 2H), 0.88 (s, 9H), 0.05 (s, 6H), LC-MS (ESI⁺) m/z 501.2 (M+H)⁺.

### Step 2—3-[4-[4-(2-Hydroxyethyl)-1-piperidyl]-3-methyl-2-oxo-benzimidazol-1-yl]piperidine-2,6-dione

To a solution of 3-[4-[4-[2-[tert-butyl(dimethyl)silyl]oxyethyl]-1-piperidyl]-3-methyl-2-oxo-benzimidazol-1-yl]piperidine-2,6-dione (400 mg, 798 umol) in a mixture solvent of ACN (4.00 mL) and H₂O (0.5 mL) was added TFA (1.54 g, 13.5 mmol). The mixture was stirred at 25° C. for 1 hr. On completion, the mixture was concentrated in vacuo. The mixture was then diluted with H₂O (10 mL) and extracted with EA (3×10 mL). The organic layer was washed with brine (2×10 mL) and dried over anhydrous Na₂SO₄, filtered and concentrated in vacuo to give the title compound (280 mg, 90% yield) as yellow solid. ¹H NMR (400 MHz, DMSO-d₆) δ 11.08 (s, 1H), 7.07-6.78 (m, 3H), 5.40-5.30 (m, 1H), 4.38 (t, J=5.2 Hz, 1H), 3.62 (s, 3H), 3.52-3.44 (m, 2H), 3.15-3.05 (m, 2H), 2.95-2.81 (m, 1H), 2.75-2.58 (m, 4H), 2.04-1.94 (m, 1H), 1.84-1.71 (m, 2H), 1.59-1.26 (m, 5H), LC-MS (ESI⁺) m/z 387.1 (M+H)⁺.

### Step 3—2-[1-[1-(2,6-Dioxo-3-piperidyl)-3-methyl-2-oxo-benzimidazol-4-yl]-4-piperidyl]acetaldehyde

To a solution of 3-[4-[4-(2-hydroxyethyl)-1-piperidyl]-3-methyl-2-oxo-benzimidazol-1-yl]piperidine-2,6-dione (100 mg, 258.7 umol) in DCM (3.00 mL) was added DMP (164 mg, 388 umol) and NaHCO₃ (108 mg, 1.29 mmol). The mixture was stirred at 20° C. for 1 hr. On completion, the mixture was diluted with DCM (15 mL), quenched with saturated Na₂S₂O₃ (15 mL) and washed with saturated NaHCO₃ (2×15 mL). The organic layer was dried over anhydrous Na₂SO₄, filtered and concentrated in vacuo to give the title compound (95 mg, 95% yield) as yellow solid. ¹H NMR (400 MHz, DMSO-d₆) δ 11.08 (s, 1H), 9.75-9.65 (m, 1H), 7.02-6.78 (m, 3H), 5.38-5.28 (m, 1H), 3.61 (s, 3H), 3.15-3.05 (m, 2H), 2.94-2.80 (m, 1H), 2.77-2.64 (m, 3H), 2.64-2.58 (m, 1H), 2.46-2.40 (m 2H), 2.06-1.89 (m, 2H), 1.79-1.72 (m, 2H), 1.50-1.35 (m, 2H), LC-MS (ESI⁺) m/z 385.1 (M+H)⁺.

### Synthesis of Tert-butyl-dimethyl-[2-(4-piperidyl)ethoxy]silane (Intermediate LB)

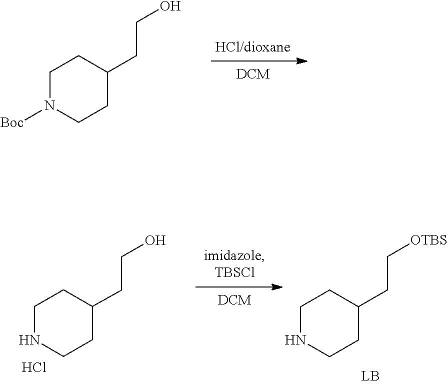 [SMILES: CC(C)(C)OC(=O)N1CCC(CCO)CC1 ; OCCC1CCNCC1 ; CC(C)(C)[Si](C)(C)OCCC1CCNCC1 ; Cl]

### Step 1—2-(4-Piperidyl)ethanol

To a solution of tert-butyl 4-(2-hydroxyethyl)piperidine-1-carboxylate (5.00 g, 21.8 mmol, CAS#198892-80-7) in DCM (50.0 mL) was added HCl/dioxane (4.00 M, 50.0 mL). The mixture was stirred at 20° C. for 0.5 hr. On completion, the mixture was concentrated in vacuo. The mixture was diluted with MeOH (50 mL) and stirred with basic ion exchange resin for 1 hr. Then the mixture was filtered and the filtrate was concentrated in vacuo to give the title compound (2.8 g, 99% yield) as yellow oil. ¹H NMR (400 MHz, DMSO-d₆) δ 4.49-4.33 (m, 1H), 3.52-3.46 (m, 2H), 3.21-3.16 (m, 2H), 2.85-2.70 (m, 2H), 1.80-1.70 (m, 2H), 1.67-1.54 (m, 1H), 1.51-1.30 (m, 2H), 1.30-1.13 (m, 2H).

### Step 2—Tert-butyl-dimethyl-[2-(4-piperidyl)ethoxy]silane

To a solution of 2-(4-piperidyl) ethanol (2.80 g, 21.6 mmol) in DCM (30.0 mL) was added TBSCl (3.92 g, 26.0 mmol) and imidazole (2.95 g, 43.3 mmol). The mixture was stirred at 20° C. for 16 hrs. On completion, the mixture was diluted with DCM (50 mL) and washed with H₂O (3×70 mL). The organic layers were washed with brine (3×50 mL) dried over anhydrous Na₂SO₄, filtered and concentrated in vacuo to give the title compound (3 g, 56% yield) as yellow oil. ¹H NMR (400 MHz, CDCl₃) δ 3.66 (t, J=6.4 Hz, 2H), 3.30-3.20 (m, 2H), 2.79-2.62 (m, 2H), 1.83-1.73 (m, 2H), 1.70-1.55 (m, 1H), 1.52-1.45 (m, 2H), 1.43-1.29 (m, 2H), 0.92 (s, 9H), 0.10 (s, 6H).

### Synthesis of 6-Chloro-8-cyclopentyl-2-[2-methyl-4-(4-piperidylsulfonyl)anilino]pyrido[2,3-d]pyrimidin-7-one (Intermediate LG)

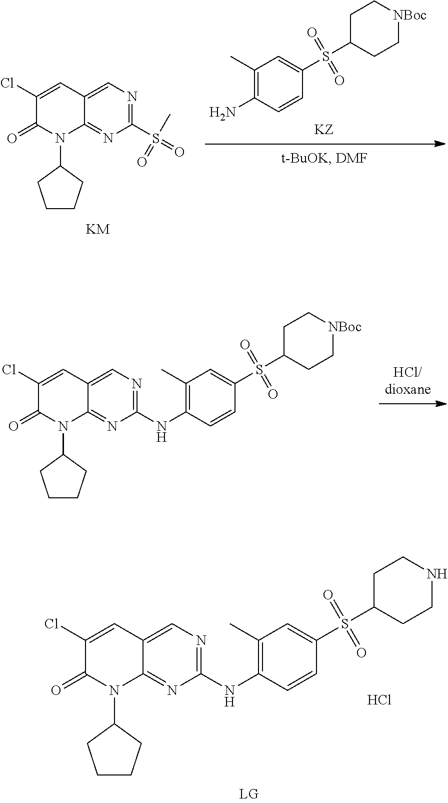

### Step 1—Tert-butyl 4-[4-[(6-chloro-8-cyclopentyl-7-oxo-pyrido[2,3-d]pyrimidin-2-yl)amino]-3-methyl -phenyl]sulfonylpiperidine-1-carboxylate

To a solution of tert-butyl 4-(4-amino-3-methyl-phenyl)sulfonylpiperidine-1-carboxylate (200 mg, 564 umol, Intermediate KZ) in DMF (3 mL) was added t-BuOK (316 mg, 2.82 mmol) at 0° C., then 6-chloro-8-cyclopentyl-2-methylsulfonyl-pyrido[2,3-d]pyrimidin-7-one (184 mg, 564 umol, Intermediate KM) was added and the mixture was stirred at 25° C. for 1 hr. On completion, the mixture was filtered to give a residue. The residue was purified by prep-HPLC (column: Phenomenex luna C18 150*25 mm*10 um; mobile phase: [water (FA)-ACN]; B %: 52%-82%, 9 min) to give the title compound (95.0 mg, 27% yield) as a yellow solid. LC-MS (ESI⁺) m/z 602.3 (M+H)⁺.

### Step 2—6-Chloro-8-cyclopentyl-2-[2-methyl-4-(4-piperidylsulfonyl)anilino]pyrido[2,3-d]pyrimidin-7-one

To a solution of tert-butyl 4-[4-[(6-chloro-8-cyclopentyl-7-oxo-pyrido[2,3-d]pyrimidin-2-yl)amino]-3-methyl-phenyl]sulfonylpiperidine-1-carboxylate (35.0 mg, 58.1 umol) was added HCl/dioxane (1 mL), then the mixture was stirred at 25° C. for 1 hr. On completion, the mixture was concentrated in vacuo to give the title compound (31.0 mg, 99% yield, HCl) as a white solid. LC-MS (ESI⁺) m/z 502.0 (M+H)⁺.

### Synthesis of Tert-butyl 4-[4-[[4-chloro-5-(trifluoromethyl)pyrimidin-2-yl]amino]-3-methyl-phenyl]sulfonylpiperidine-1-carboxylate (Intermediate LH)

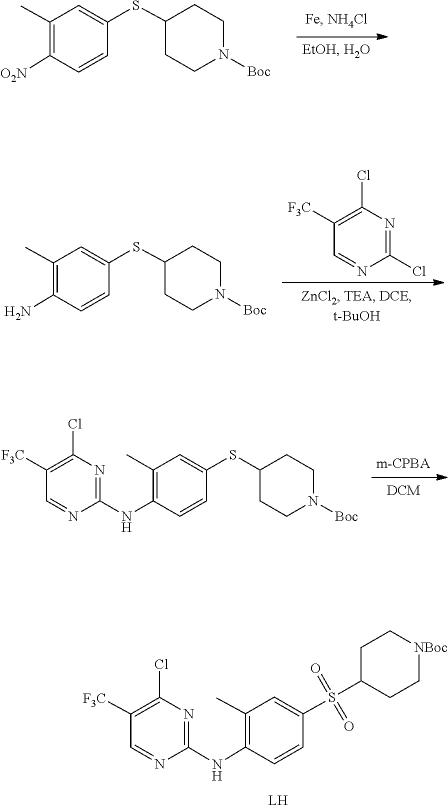 [SMILES: Cc1cc(SC2CCN(C(=O)OC(C)(C)C)CC2)ccc1[N+](=O)[O-] ; Cc1cc(SC2CCN(C(=O)OC(C)(C)C)CC2)ccc1N ; FC(F)(F)c1cnc(Cl)nc1Cl ; Cc1cc(SC2CCN(C(=O)OC(C)(C)C)CC2)ccc1Nc1ncc(C(F)(F)F)c(Cl)n1 ; Cc1cc(S(=O)(=O)C2CCN(C(=O)OC(C)(C)C)CC2)ccc1Nc1ncc(C(F)(F)F)c(Cl)n1]

Step 1—Tert-butyl 4-(4-amino-3-methyl-phenyl)sulfanylpiperidine-1-carboxylate

To a solution of tert-butyl 4-(3-methyl-4-nitro-phenyl)sulfanylpiperidine-1-carboxylate (5.00 g, 14.1 mmol, synthesized via Step 1 of Intermediate KZ) in EtOH (50 mL) and H₂O (10 mL) was added Fe (4.75 g, 85.1 mmol) and NH₄Cl (7.59 g, 141 mmol). Then the mixture was stirred at 80° C. for 2 hrs under N₂ atmosphere. On completion, the mixture was filtered and the filtrate was concentrated in vacuo to give the residue. The residue was purified by column chromatography (SiO₂, PE:EA=10:1 to 3:1) to give the title compound (4.00 g, 87% yield) as a yellow solid. ¹H NMR (400 MHz, DMSO-d₆) δ 7.03 (d, J=1.2 Hz, 1H), 6.99 (dd, J=2.0, 8.0 Hz, 1H), 6.56 (d, J=8.0 Hz, 1H), 5.07 (s, 2H), 3.80 (d, J=13.2 Hz, 2H), 2.96-2.88 (m, 1H), 2.81 (s, 2H), 2.02 (s, 3H), 1.79-1.72 (m, 2H), 1.36 (s, 9H), 1.29-1.20 (m, 2H).

### Step 2—Tert-butyl 4-[4-[[4-chloro-5-(trifluoromethyl)pyrimidin-2-yl]amino]-3-methyl-phenyl]sulfanylpiperidine-1-carboxylate

To a solution of 2,4-dichloro-5-(trifluoromethyl)pyrimidine (200 mg, 921 umol, CAS #3932-97-6) in mixture solvent of DCE (4 mL) and t-BuOH (4 mL) was added ZnCl₂ (150 mg, 1.11 mmol) at 0° C. After 1 hour, a solution of tert-butyl 4-(4-amino-3-methyl-phenyl)sulfanylpiperidine-1-carboxylate (297 mg, 921 umol) and TEA (102 mg, 1.01 mmol) in mixture solvent of DCE (2 mL) and t-BuOH (2 mL) was dropwise into the above solution. The mixture was then stirred at 25° C. for 15 hrs. On completion, the mixture was diluted with H₂O (20 mL), and extracted with EA (3×30 mL). The organic layer was washed with brine (2×30 mL), dried over anhydrous sodium sulfate, filtered and concentrated in vacuo. The residue was purified by column chromatography (SiO₂, PE/EA=50/1 to 10/1) to give the title compound (330 mg, 71% yield) as a yellow solid. ¹H NMR (400 MHz, DMSO-d₆) δ 10.09 (s, 1H), 8.68 (s, 1H), 7.39-7.21 (m, 3H), 3.82 (d, J=13.6 Hz, 2H), 3.39 (d, J=4.0 Hz, 2H), 2.91 (d, J=14.4 Hz, 2H), 2.50 (s, 1H), 2.19 (s, 3H), 1.91-1.84 (m, 2H), 1.38 (s, 9H). LCMS (ESI⁺) m/z 503.1 (M+H)⁺.

### Step 3—tert-butyl 4-[4-[[4-chloro-5-(trifluoromethyl)pyrimidin-2-yl]amino]-3-methyl-phenyl]sulfonylpiperidine-1-carboxylate

To a solution of tert-butyl 4-[4-[[4-chloro-5-(trifluoromethyl)pyrimidin-2-yl]amino]-3-methyl-phenyl] sulfanylpiperidine-1-carboxylate (320 mg, 636 umol) in DCM (5 mL) was added m-CPBA (516 mg, 2.54 mmol, 85% solution). The mixture was then stirred at 25° C. for 2 hrs. On completion, the reaction mixture was partitioned between Na₂SO₃ (20 mL) and DCM (20 mL). The organic phase was separated, washed with brine (20 mL), dried over anhydrous Na₂SO₄, filtered and concentrated in vacuo to give the title compound (330 mg, 96% yield) as a yellow solid. LCMS (ESI⁺) m/z 557.1 (M+23)+.

### Synthesis of N-[2-methyl-4-(4-piperidylsulfonyl)phenyl]-4-(1-methylpyrazol-4-yl)-5-(trifluoromethyl) pyrimidin-2-amine (Intermediate LI)

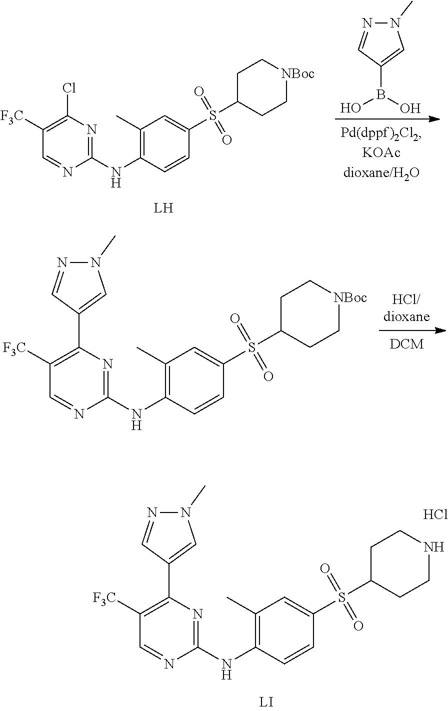

### Step 1—Tert-butyl 4-[3-methyl-4-[[4-(1-methylpyrazol-4-yl)-5-(trifluoromethyl)pyrimidin-2-yl]amino]phenyl]sulfonylpiperidine-1-carboxylate

A mixture of tert-butyl 4-[4-[[4-chloro-5-(trifluoromethyl)pyrimidin-2-yl]amino]-3-methyl-phenyl] sulfonylpiperidine-1-carboxylate (300 mg, 560.77 umol, Intermediate LH), (1-methylpyrazol-4-yl)boronic acid (84.7 mg, 672 umol, CAS #847818-55-7), cyclopentyl(diphenyl)phosphane;dichloropalladium;iron (41.0 mg, 56.0 umol), and KOAc (165 mg, 1.68 mmol) in a mixture solution of dioxane (4 mL) and H₂O (1 mL) was degassed and purged with N₂ three times, and then the mixture was stirred at 90° C. for 16 hrs under N₂ atmosphere. The reaction mixture was concentrated in vacuo to give a residue. The residue was purified by prep-HPLC (column: Phenomenex luna C18 150*25 mm*10 um; mobile phase: [water(FA)-ACN]; B %: 46%-76%, 10 min) to give the title compound (100 mg, 30% yield) as a yellow solid. ¹H NMR (400 MHz, DMSO-d₆) δ 9.70 (s, 1H), 8.74 (s, 1H), 8.23 (s, 1H), 7.97 (d, J=8.4 Hz, 1H), 7.93 (s, 1H), 7.76-7.66 (m, 2H), 4.07-3.97 (m, 2H), 3.93 (s, 3H), 3.49-3.42 (m, 1H), 2.83-2.68 (m, 2H), 2.39 (s, 3H), 1.85 (d, J=11.2 Hz, 2H), 1.37 (s, 11H). LCMS (ESI⁺) m/z 581.1 (M+H)⁺.

### Step 2—N-[2-methyl-4-(4-piperidylsulfonyl)phenyl]-4-(1-methylpyrazol-4-yl)-5-(trifluoromethyl) pyrimidin-2-amine

To a solution of tert-butyl 4-[3-methyl-4-[[4-(1-methylpyrazol-4-yl)-5-(trifluoromethyl) pyrimidin-2-yl]amino]phenyl]sulfonylpiperidine-1-carboxylate (80.0 mg, 137 umol) in DCM (0.5 mL) was added HCl/dioxane (4 M, 0.5 mL). The mixture was stirred at 25° C. for 0.5 hour. On completion, the reaction mixture was concentrated in vacuo to give the title compound (70.0 mg, 98% yield, HCl) as a white solid. LCMS (ESI⁺) m/z 481.0 (M+H)⁺.

### Synthesis of 4-[3-[4-[(6-Chloro-8-cyclopentyl-7-oxo-pyrido[2,3-d]pyrimidin-2-yl)amino]-3-methyl-phenyl]sulfonylbutoxy]cyclohexanecarbaldehyde (Intermediate LJ)

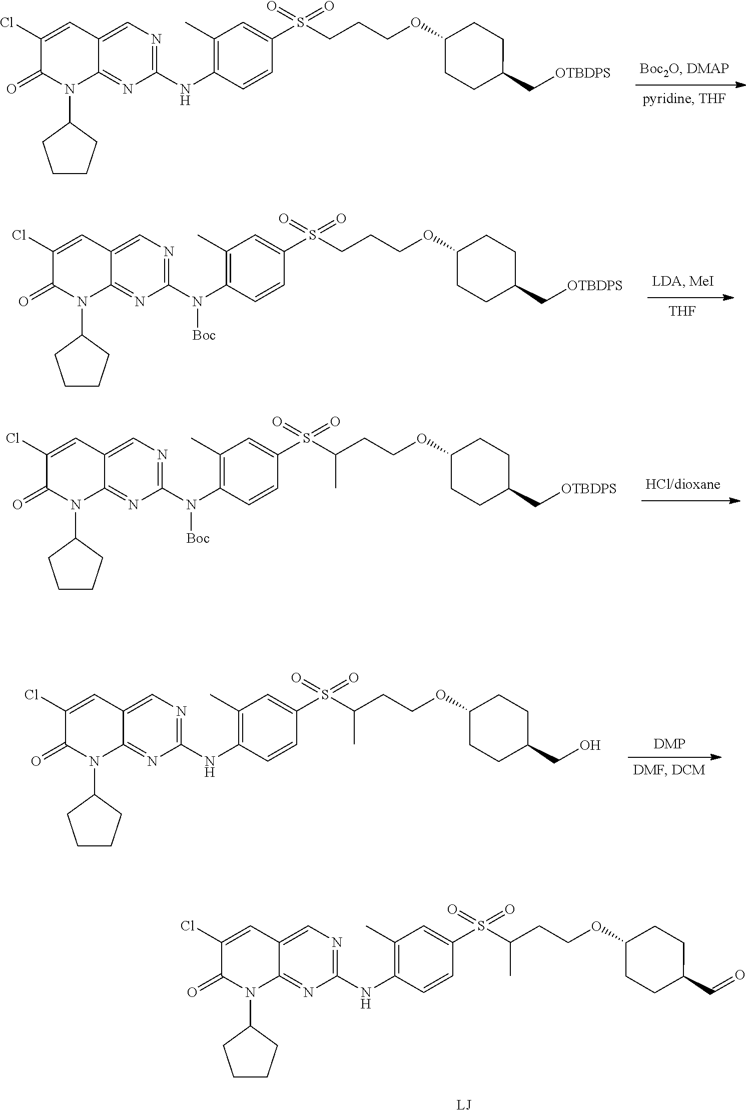

### Step 1—Tert-butyl N-[4-[3-[4-[[tert-butyl(diphenyl)silyl]oxymethyl]cyclohexoxy]propylsulfonyl]-2-methyl-phenyl]-N-(6-chloro-8-cyclopentyl-7-oxo-pyrido[2,3-d]pyrimidin-2-yl)carbamate

To a solution of 2-[4-[3-[4-[[tert-butyl(diphenyl)silyl]oxymethyl]cyclohexoxy]propylsulfonyl]-2-methyl-anilino]-6-chloro-8-cyclopentyl-pyrido[2,3-d]pyrimidin-7-one (18.0 mg, 21.7 umol, synthesized via Step 1 of Intermediate CX) in THF (1 mL) was added pyridine (3.44 mg, 43.5 umol, 3.51 uL), Boc₂O (7.12 mg, 32.6 umol, 7.50 uL) and DMAP (531 ug, 4.35 umol). The mixture was stirred at 60° C. for 2 hrs. On completion, the mixture was diluted with citric acid monohydrate (2 mL) and extracted with DCM (3×3 mL). The combined organic layers were washed with brine (2×3 mL), dried over anhydrous MgSO₄, filtered and concentrated in vacuo to give a residue. The residue was purified by prep-TLC (SiO₂, PE:EA=1:1, Rf=0.6) to give the title compound (20 mg, 94% yield) as yellow solid. ¹H NMR (400 MHz, DMSO-d₆) δ 8.92 (s, 1H), 8.31 (s, 1H), 7.90 (d, J=2.0 Hz, 1H), 7.80-7.73 (m, 1H), 7.60-7.56 (m, 4H), 7.47-7.40 (m, 7H), 5.63-5.53 (m, 1H), 5.27-5.04 (m, 2H), 3.44-3.40 (m, 4H), 3.10-3.01 (m, 1H), 2.20 (s, 3H), 2.08-2.02 (m, 2H), 1.95-1.88 (m, 2H), 1.76-1.65 (m, 7H), 1.48-1.45 (m, 2H), 1.42 (s, 9H), 1.23 (s, 2H), 1.08-1.00 (m, 2H), 0.98 (s, 9H), 0.97-0.88 (m, 2H). LC-MS (ESI⁺) m/z 927.4 (M+H)⁺.

### Step 2—Tert-butyl N-[4-[3-[4-[[tert-butyl(diphenyl)silyl]oxymethyl]cyclohexoxy]-1-methyl-propyl]sulfonyl-2-methyl-phenyl]-N-(6-chloro-8-cyclopentyl-7-oxo-pyrido[2,3-d]pyrimidin-2-yl)carbamate

A mixture of tert-butyl N-[4-[3-[4-[[tert-butyl(diphenyl)silyl]oxymethyl]cyclohexoxy]propylsulfonyl]-2-methyl-phenyl]-N-(6-chloro-8-cyclopentyl-7-oxo-pyrido[2,3-d]pyrimidin-2-yl)carbamate (20.0 mg, 21.5 umol) in THF (1 mL) was degassed and purged with N₂ three times at 25° C. Then LDA (2 M, 53.9 uL) was added dropwise at 25° C. After 10 minutes, MeI (30.6 mg, 215 umol, 13.5 uL) was added dropwise at 25° C. The mixture was stirred at 25° C. for 14 hrs. On completion, the reaction mixture was quenched with H₂O (0.1 mL) at 0° C., and then diluted with H₂O (2 mL) and extracted with EA (3×3 mL). The combined organic layers were washed with brine (2×3 mL), dried over anhydrous Na₂SO₄ filtered and concentrated in vacuo to give the title compound (5 mg, 25% yield) as a yellow solid. LC-MS (ESI⁺) m/z 941.2 (M+H)⁺.

### Step 3—6-Chloro-8-cyclopentyl-2-[4-[3-[4-(hydroxymethyl)cyclohexoxy]-1-methyl-propyl]sulfonyl-2-methyl-anilino]pyrido[2,3-d]pyrimidin-7-one

A solution of tert-butyl N-[4-[3-[4-[[tert-butyl(diphenyl)silyl]oxymethyl]cyclohexoxy]-1-methyl-propyl]sulfonyl-2-methyl-phenyl]-N-(6-chloro-8-cyclopentyl-7-oxo-pyrido[2,3-d]pyrimidin-2-yl)carbamate (270 mg, 192 umol) in HCl/dioxane (1 mL) was stirred at 25° C. for 14 hrs. On completion, the mixture was filtered and concentrated in vacuo to give a residue. The residue was purified by prep-TLC (SiO₂, PE:EA=1:1, Rf=0.5) to give the title compound (44 mg, 35% yield) as a yellow solid. ¹H NMR (400 MHz, DMSO-d₆) δ 9.72 (s, 1H), 8.77 (s, 1H), 8.19 (s, 1H), 7.82 (d, J=8.4 Hz, 1H), 7.74 (s, 1H), 7.71-7.64 (m, 1H), 5.82-5.66 (m, 1H), 4.43-4.27 (m, 1H), 3.44 (dd, J=3.2, 8.8 Hz, 1H), 3.16 (t, J=5.6 Hz, 2H), 3.11-3.03 (m, 1H), 2.37 (s, 3H), 2.10 (d, J=9.6 Hz, 2H), 1.94-1.87 (m, 2H), 1.73-1.67 (m, 6H), 1.50-1.43 (m, 3H), 1.25 (d, J=12.0 Hz, 4H), 1.19 (d, J=6.8 Hz, 3H), 1.08-1.00 (m, 2H), 0.89-0.82 (m, 2H). LC-MS (ESI⁺) m/z 603.1 (M+H)⁺.

### Step 4—4-[3-[4-[(6-Chloro-8-cyclopentyl-7-oxo-pyrido[2,3-d]pyrimidin-2-yl)amino]-3-methyl-phenyl]sulfonylbutoxy]cyclohexanecarbaldehyde

To a solution of 6-chloro-8-cyclopentyl-2-[4-[3-[4-(hydroxymethyl)cyclohexoxy]-1-methyl-propyl]sulfonyl-2-methyl-anilino]pyrido[2,3-d]pyrimidin-7-one (44.0 mg, 72.9 umol) in DCM (1 mL) and DMF (0.2 mL) was added DMP (46.4 mg, 109 umol, 34.0 uL). The mixture was stirred at 25° C. for 1.5 hours. On completion, the reaction mixture was quenched with Na₂S₂O₃ (0.5 mL) at 25° C., and then diluted with NaHCO₃ (5 mL) and extracted with DCM (3×3 mL). The combined organic layers were washed with brine (2×2 mL), dried over anhydrous Na₂SO₄, filtered and concentrated in vacuo to give the title compound (43 mg, 98% yield) as yellow oil. LC-MS (ESI⁺) m/z 601.3 (M+H)⁺.

Intermediates G, H, M, DQ, EG, HB, KM, and HN, below, were prepared according to PCT/US2022/028076, the entirety of which is herein incorporated by reference.

### [1-[(4-Methoxyphenyl) methyl]-2,6-dioxo-3-piperidyl] trifluorometh anesulfonate (Intermediate G)

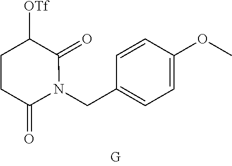 [SMILES: COc1ccc(CN2C(=O)CCC(OS(=O)(=O)C(F)(F)F)C2=O)cc1]

### 3-(4-bromo-3-methyl-2-oxo-2,3-dihydro-1H-benzo[d]imidazol-1-yl)piperidine-2,6-dione (Intermediate H)

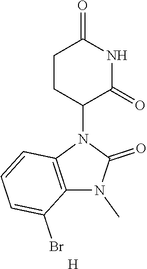

### 4-(Benzylthio)-2-methylaniline (Intermediate M)

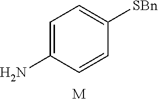

### 3-[5-[1-[[4-(2-Aminoethoxy)cyclohexyl]methyl]-4-piperidyl]-3-methyl-2-oxo-benzimidazol-1-yl]piperidine-2,6-dione (Intermediate DQ)

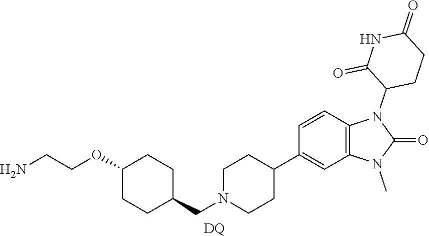

### 3-[3-Methyl-4-[4-(methylamino)-1-piperidyl]-2-oxo-benzimidazol-1-yl]piperidine-2,6-dione (Intermediate EG)

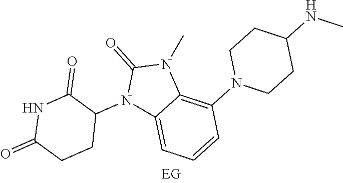 [SMILES: CNC1CCN(c2cccc3c2n(C)c(=O)n3C2CCC(=O)NC2=O)CC1]

### 7′-Cyclopentyl-2′-methylsulfonyl-spiro[cyclopropane-1,5′-pyrrolo[2,3-d]pyrimidine]-6′-one (Intermediate HB)

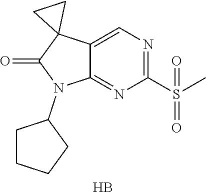

### 6-Chloro-8-cyclopentyl-2-methylsulfonyl-pyrido[2,3-d]pyrimidin-7-one (Intermediate KM)

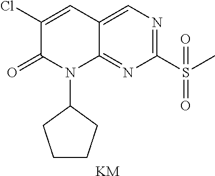

### 8-Cyclopentyl-2-methylsulfanyl-pyrido[2,3-d]pyrimidin-7-one (Intermediate HN)

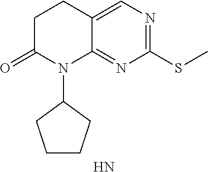

### Synthesis of N-(4-((7-Azaspiro[3.5]nonan-2-yl)sulfonyl)-2-methylphenyl)-4-((tetrahydrofuran-3-yl)oxy) -5-(trifluoromethyl)pyrimidin-2-amine (Intermediate MT)

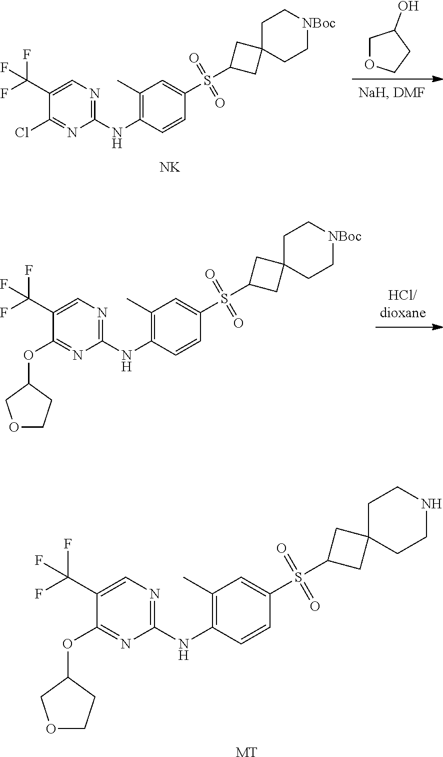

### Step 1—Tert-butyl 2-((3-methyl-4-((4-((tetrahydrofuran-3-yl)oxy)-5-(trifluoromethyl)pyrimidin-2-yl)amino)phenyl)sulfonyl)-7-azaspiro[3.5]nonane-7-carboxylate

A mixture of tert-butyl 2-[4-[[4-chloro-5-(trifluoromethyl)pyrimidin-2-yl]amino]-3-methyl-phenyl]-sulfonyl-7-azaspiro[3.5]nonane-7-carboxylate (234 mg, 406 μmol, Intermediate NK) in DMF was added NaH (32.5 mg, 813 mol, 60% dispersion in mineral oil) at 0° C. The reaction mixture was stirred at 0° C. for 0.5 hr and then tetrahydrofuran-3-ol (53.7 mg, 610 mol, CAS #453-20-3) was added. The reaction mixture was stirred at 100° C. for 1 hr. On completion, the mixture was quenched with water (1 mL) and the filtrate was diluted with water (5 mL), then extracted with EA (10 mL×3). The combined organic layers were concentrated in vacuo to give the residue. The residue was purified by reverse-phase (0.1% FA condition) to give the title compound (150 mg, 58% yield) as a yellow solid. LC-MS (ESI⁺) m/z 627.2 (M+H)⁺.

### Step 2—N-(4-((7-Azaspiro[3.5]nonan-2-yl)sulfonyl)-2-methylphenyl)-4-((tetrahydrofuran-3-yl)oxy) -5-(trifluoromethyl)pyrimidin-2-amine

A solution of tert-butyl 2-[3-methyl-4-[[4-tetrahydrofuran-3-yloxy-5-(trifluoromethyl)pyrimidin-2-yl]amino]phenyl]sulfonyl-7-azaspiro[3.5]nonane-7-carboxylate (150 mg, 239 mol) in HCl/dioxane (4M, 5 mL) was stirred at 25° C. for 0.5 hr. On completion, the mixture was concentrated in vacuo to give the title compound (121 mg, 96% yield, HCl) as a yellow oil. LC-MS (ESI⁺) m/z 527.2 (M+H)⁺.

### Synthesis of 1-(4-fluoro-1-(1-(4-methoxybenzyl)-2,6-dioxopiperidin-3-yl)-3-methyl-2-oxo-2,3-dihydro-1H-benzo[d]imidazol-5-yl)piperidine-4-carbaldehyde (Intermediate MU)

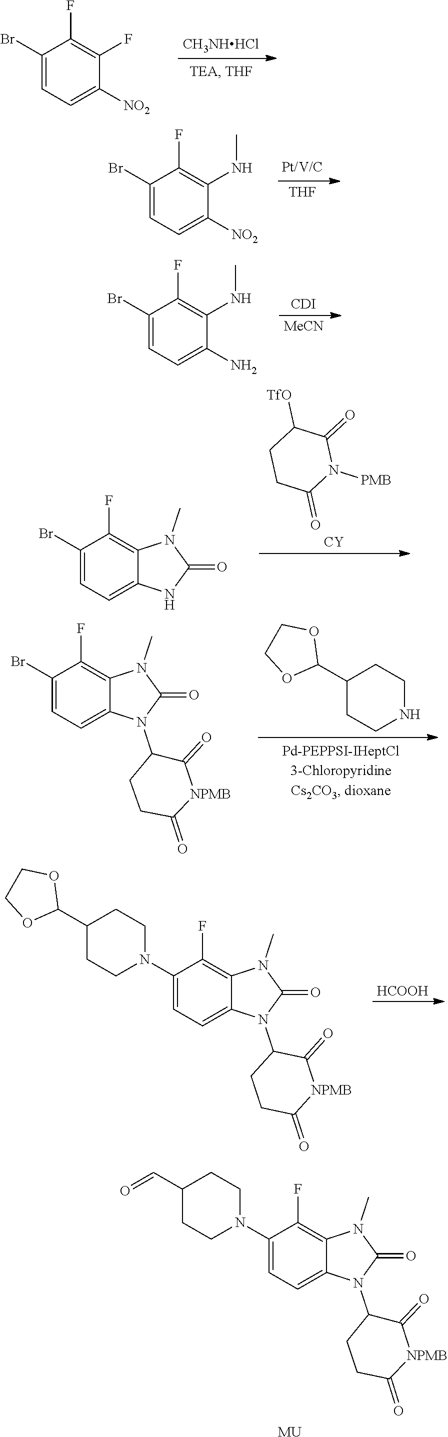

### Step 1—3-Bromo-2-fluoro-N-methyl-6-nitroaniline

To a solution of methanamine hydrochloride (7.09 g, 105 mmol) in THF (150 mL) was added TEA (12.7 g, 126 mmol, 17.5 mL) at 0° C., and the mixture was stirred for 10 minutes. Then 1-bromo-2,3-difluoro-4-nitro-benzene (5 g, 20 mmol, CAS #1003708-24-4) was added into reaction liquid. The reaction was stirred for 4 hrs at 25° C. On completion, the reaction mixture was quenched with H₂O (50 mL) under stirring. The residue was diluted with water (500 mL) and extracted with EA (50 mL×3). The combined organic layer was dried over Na₂SO₄, filtered and concentrated in vacuo to give the title compound (5 g, 95% yield) as yellow solid. LC-MS (ESI⁺) m/z 249.1 (M+H)⁺.

### Step 2—5-Bromo-6-fluoro-N1-methylbenzene-1,2-diamine

To a 3-bromo-2-fluoro-N-methyl-6-nitro-aniline (200 mg, 803 umol) in THF (10 mL) was added Pt/V/C (41.93 mg, 160 umol) under N₂ atmosphere. The suspension was degassed and purged with H₂ for three times. The mixture was stirred under H₂ (15 Psi) at 25° C. for 0.5 hr. On completion, the reaction mixture was filtered and concentrated in vacuo to give the title compound (170 mg, 97% yield) as a black brown oil. LC-MS (ESI⁺) m/z 218.8 (M+H)⁺.

### Step 3—6-Bromo-7-fluoro-1-methyl-1,3-dihydro-2H-benzo[d]imidazol-2-one

To a solution of 4-bromo-3-fluoro-N2-methyl-benzene-1,2-diamine (170 mg, 776 umol) in MeCN (10 mL) was added CDI (188 mg, 1.16 mmol) at 25° C., then the reaction mixture was stirred at 85° C. for 12 hrs. On completion, the reaction mixture was concentrated in vacuo then poured into ice water (3 mL) to give the title compound (160 mg, 39% yield) as brown solid. LC-MS (ESI⁺) m/z 249.1 (M+H)⁺.

### Step 4—3-(5-Bromo-4-fluoro-3-methyl-2-oxo-2,3-dihydro-1H-benzo[d]imidazol-1-yl)-1-(4-methoxybenzyl)piperidine-2,6-dione

To a solution of 5-bromo-4-fluoro-3-methyl-1H-benzimidazol-2-one (160 mg, 652 umol) in THF (15 mL) was added t-BuOK (109 mg, 979 umol). The mixture was stirred at −10° C. for 30 mins. Then a solution of [1-[(4-methoxyphenyl)methyl]-2,6-dioxo-3-piperidyl] trifluoromethanesulfonate (298 mg, 783 umol, Intermediate CY) in THF (15 mL) was added dropwise to the mixture, and the reaction mixture was stirred at −10° C. for 30 mins. On completion, the reaction mixture was quenched with H₂O (0.2 mL) under stirring, then the reaction mixture was diluted with H₂O (10 mL) and extracted with DCM (10×3 mL). The combined organic layer was washed with brine (2×10 mL), dried over Na₂SO₄, filtered and concentrated in vacuo to give the title compound (200 mg, 64% yield) as a yellow solid. ¹H NMR (400 MHz, DMSO-d₆) δ 7.30 (dd, J=6.4, 8.4 Hz, 1H), 7.21 (d, J=8.4 Hz, 2H), 6.93 (br d, J=8.1 Hz, 1H), 6.86 (d, J=8.8 Hz, 2H), 5.57 (dd, J=5.6, 13.2 Hz, 1H), 4.87-4.70 (m, 2H), 3.73 (s, 3H), 3.50 (d, J=1.8 Hz, 3H), 3.08-3.01 (m, 1H), 2.85 (br d, J=2.4 Hz, 1H), 2.81-2.65 (m, 2H); LC-MS (ESI⁺) m/z 476.1 (M+H)⁺.

### Step 5—3-(5-(4-(1,3-Dioxolan-2-yl)piperidin-1-yl)-4-fluoro-3-methyl-2-oxo-2,3-dihydro-1H-benzo[d]imidazol-1-yl)-1-(4-methoxybenzyl)piperidine-2,6-dione

A mixture of 3-(5-bromo-4-fluoro-3-methyl-2-oxo-benzimidazol-1-yl)-1-[(4-methoxyphenyl)methyl] piperidine-2,6-dione (150 mg, 314 umol), 4-(1,3-dioxolan-2-yl) piperidine (49.5 mg, 314 umol), 1,3-bis[2,6-bis(1-propylbutyl)phenyl]-4,5-dichloro-2H-imidazol-1-ium-2-ide;3-chloropyridine dichloropalladium (30.6 mg, 31.4 umol), and dicesium carbonate (205 mg, 629 umol) in dioxane (4 mL) was stirred at 100° C. for 16 hrs under N₂. On completion, the reaction mixture was filtered and concentrated in vacuo to give a residue. The residue was purified by flash silica gel chromatography (SiO₂, DCM/Ethyl acetate=1/1) to give the title compound (150 mg, 86% yield) as light yellow solid. ¹H NMR (400 MHz, CDCl₃) δ 7.29 (d, J=8.4 Hz, 2H), 6.75 (d, J=8.4 Hz, 2H), 6.48 (br dd, J=2.0, 4.4 Hz, 1H), 6.09 (br d, J=8.4 Hz, 1H), 5.11-5.04 (m, 1H), 4.88 (s, 2H), 4.64 (br d, J=0.8 Hz, 1H), 3.92-3.87 (m, 2H), 3.85-3.81 (m, 2H), 3.72 (s, 1H), 3.73-3.71 (m, 1H), 3.51 (br s, 3H), 2.09 (tdd, J=2.8, 5.2, 12.9 Hz, 1H), 1.98 (s, 1H), 1.79 (br s, 2H), 1.59 (br s, 1H); LC-MS (ESI⁺) m/z 553.1 (M+H)⁺.

### Step 6—1-(4-fluoro-1-(1-(4-methoxybenzyl)-2,6-dioxopiperidin-3-yl)-3-methyl-2-oxo-2,3-dihydro-1H-benzo[d]imidazol-5-yl)piperidine-4-carbaldehyde

A solution of 3-[5-[4-(1,3-dioxolan-2-yl)-1-piperidyl]-4-fluoro-3-methyl-2-oxo-benzimidazol-1-yl]-1-[(4-methoxyphenyl)methyl]piperidine-2,6-dione (145 mg, 262 umol) in HCOOH (1.5 mL) was stirred at 70° C. for 1 hr. On completion, the mixture was concentrated in vacuo give the title compound (100 mg, 75% yield) as light yellow solid. LC-MS (ESI⁺) m/z 509.4 (M+H)⁺.

### Synthesis of 1-[1-(2,6-Dioxo-3-piperidyl)-3-methyl-2-oxo-benzimidazol-4-yl]piperidine-4-carbaldehyde (Intermediate MV)

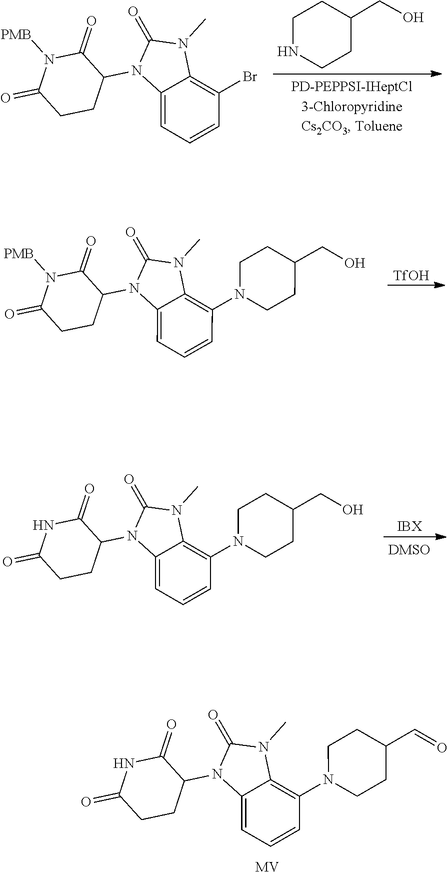

### Step 1—3-[4-[4-(Hydroxymethyl)-1-piperidyl]-3-methyl-2-oxo-benzimidazol-1-yl]-1-[(4-methoxyphenyl)methyl]piperidine-2,6-dione

A mixture of 3-(4-bromo-3-methyl-2-oxo-benzimidazol-1-yl)-1-[(4-methoxyphenyl)methyl]piperidine-2,6-dione (2 g, 4 mmol, synthesized via Steps 1-4 of Intermediate DC), 4-piperidylmethanol (752 mg, 6.56 mmol, CAS #6457-49-4), RuPhos Pd G₃ (728 mg, 872 umol), 4A molecular sieves (20 mg), RuPhos (404 mg, 872 umol) and LiHMDS (1 M, 20 mL) in toluene (20 mL) was stirred at 100° C. for 6 hrs. On completion, the reaction mixture was filtered and concentrated in vacuo to give a residue. The residue was purified by reverse phase (0.1% FA condition) and re-purified by prep-HPLC (column: Phenomenex luna C18 150*25 mm*10 um; mobile phase: [water (FA)-ACN]) to give the title compound (150 mg, 7% yield) as a brown solid. ¹H NMR (400 MHz, DMSO-d₆) δ 7.24-7.16 (m, 3H), 7.02-6.97 (m, 1H), 6.96-6.88 (m, 1H), 6.85 (d, J=8.4 Hz, 2H), 5.57-5.46 (m, 1H), 4.86-4.72 (m, 2H), 4.49 (t, J=5.2 Hz, 1H), 3.72 (s, 4H), 3.62 (s, 2H), 3.34 (s, 3H), 3.16-2.97 (m, 3H), 2.90-2.56 (m, 4H), 2.12-1.95 (m, 1H), 1.77 (d, J=10.4 Hz, 2H), 1.56-1.25 (m, 2H). LC-MS (ESI⁺) m/z 493.3 (M+H)⁺.

### Step 2—3-[4-[4-(Hydroxymethyl)-1-piperidyl]-3-methyl-2-oxo-benzimidazol-1-yl]piperidine-2,6-dione

A solution of 3-[4-[4-(hydroxymethyl)-1-piperidyl]-3-methyl-2-oxo-benzimidazol-1-yl]-1-[(4-methoxyphenyl)methyl]piperidine-2,6-dione (90 mg, 180 umol) in TfOH (0.5 mL) was stirred at 60° C. for 0.5 hr. On completion, the reaction mixture was concentrated in vacuo to give a residue. The residue was purified by prep-HPLC (column: Phenomenex luna C18 150*25 mm*10 um; mobile phase: [water (FA)-ACN]) to give the title compound (40 mg, 59% yield) as a white solid. LC-MS (ESI⁺) m/z 373.3 (M+H)⁺.

### Step 3—1-[1-(2,6-Dioxo-3-piperidyl)-3-methyl-2-oxo-benzimidazol-4-yl]piperidine-4-carbaldehyde

To a solution of 3-[4-[4-(hydroxymethyl)-1-piperidyl]-3-methyl-2-oxo-benzimidazol-1-yl]piperidine-2,6-dione (30 mg, 80 umol) in DMSO (0.5 mL) was added IBX (67.6 mg, 241 umol), then the mixture was stirred at 60° C. for 1 hr. On completion, the reaction mixture was quenched by saturated Na₂S₂O₃ (5 mL) and saturated NaHCO₃ (5 mL) at 25° C., and then stirred for 30 minutes. Then the organic layer was separated and concentrated in vacuo to give the crude product. The mixture was diluted with water (5 mL) and extracted with EA (10 mL×3). The combined organic layer was dried over Na₂SO₄, filtered and concentrated in vacuo to give title compound (26 mg, 87% yield) as a yellow solid. LC-MS (ESI⁺) m/z 371.1 (M+H)⁺.

### Synthesis of Tert-butyl 2-((4-((4-((1s,4s)-4-hydroxy-4-methylcyclohexyl)-5-(trifluoromethyl)pyrimidin-2-yl)amino)-3-methylphenyl)thio)-7-azaspiro[3.5]nonane-7-carboxylate (Intermediate MW) and tert-butyl 2-((4-((4-((1r,4r)-4-hydroxy-4-methylcyclohexyl)-5-(trifluoromethyl)pyrimidin-2-yl)amino)-3-methylphenyl)thio)-7-azaspiro[3.5]nonane-7-carboxylate (Intermediate MX)

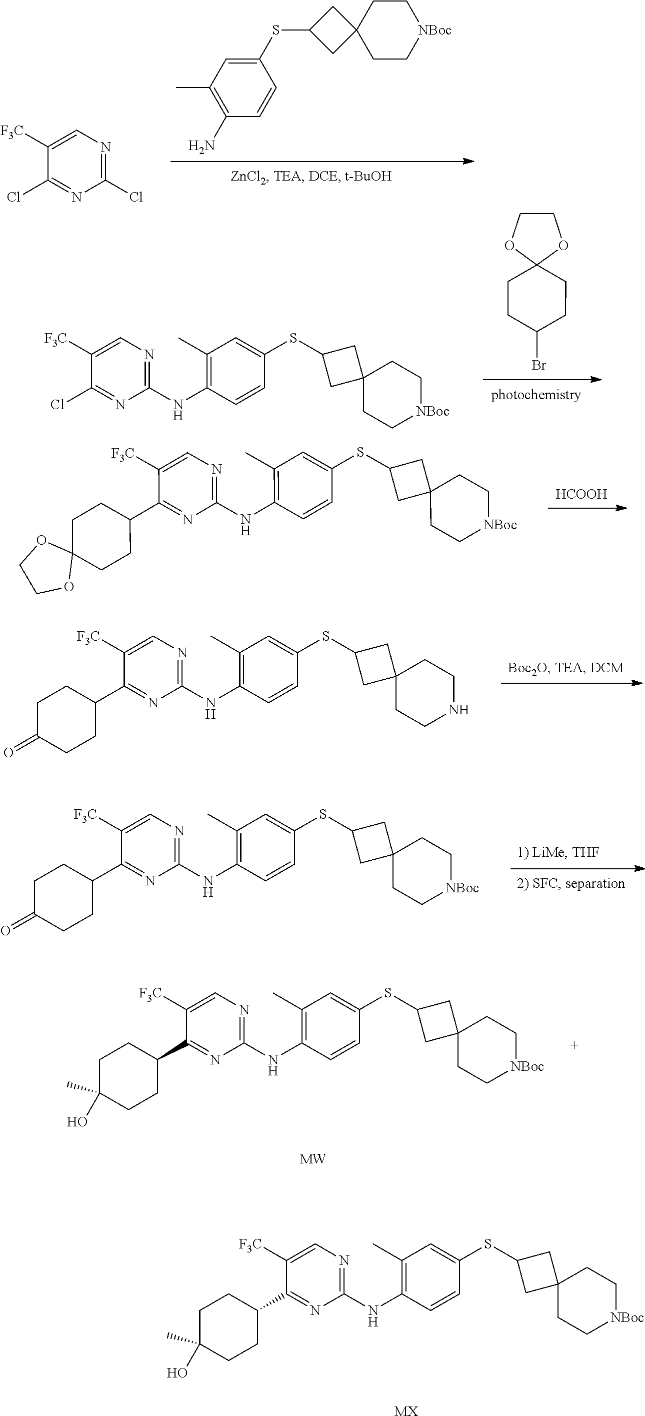

Step 1—Tert-butyl 2-[4-[[4-chloro-5-(trifluoromethyl)pyrimidin-2-yl]amino]-3-methyl-phenyl] sulfanyl-7-azaspiro[3.5]nonane-7-carboxylate

To a solution of 2,4-dichloro-5-(trifluoromethyl)pyrimidine (1.20 g, 5.52 mmol) in t-BuOH (10 mL) and DCE (10 mL) was added dropwise ZnCl₂ (1 M, 6.62 mL) at 0° C. for 30 mins. Then tert-butyl 2-(4-amino-3-methyl-phenyl)sulfanyl-7-azaspiro[3.5]nonane-7-carboxylate (2.00 g, 5.52 mmol, synthesized via Step 1 of Intermediate PF) in t-BuOH (10 mL), DCE (10 mL), and TEA (614 mg, 6.07 mmol, 844 uL) was added dropwise at 0° C. The resulting mixture was stirred at 25° C. for 14 hrs. On completion, the mixture was diluted with H₂O (30 mL), and extracted with EA (2×10 mL). The organic layers were washed with brine (2×10 mL), dried over anhydrous Na₂SO₄, filtered and concentrated in vacuo to give a residue. The residue was purified by column chromatography (SiO₂, Petroleum ether/Ethyl acetate=100/1 to 20/1) to give the title compound (2.60 g, 37% yield) as a white solid. ¹H NMR (400 MHz, DMSO-d₆) δ 10.05 (s, 1H), 8.66 (s, 1H), 7.26 (d, J=8.4 Hz, 1H), 7.13 (d, J=1.6 Hz, 1H), 7.08-7.04 (m, 1H), 4.00-3.91 (m, 1H), 3.25 (d, J=5.2 Hz, 2H), 3.17 (d, J=5.6 Hz, 2H), 2.42-2.36 (m, 2H), 2.16 (s, 3H), 1.79-1.72 (m, 2H), 1.58-1.52 (m, 2H), 1.49-1.44 (m, 2H), 1.39-1.36 (m, 9H). LC-MS (ESI⁺) m/z 565.3 (M+Na)+.

### Step 2—Tert-butyl 2-[4-[[4-(1,4-dioxaspiro[4.5]decan-8-yl)-5-(trifluoromethyl)pyrimidin-2-yl]amino]-3-methyl-phenyl]sulfanyl-7-azaspiro[3.5]nonane-7-carboxylate

To a solution of tert-butyl 2-[4-[[4-chloro-5-(trifluoromethyl)pyrimidin-2-yl]amino]-3-methyl-phenyl]sulfanyl-7-azaspiro[3.5]nonane-7-carboxylate (1.20 g, 2.21 mmol) and 8-bromo-1,4-dioxaspiro[4.5]decane (635 mg, 2.87 mmol, CAS #68278-51-3) in DCE (15 mL) was added Ir[dF(CF₃)ppy]₂(dtbpy)(PF₆) (24.7 mg, 22.1 umol), TTMSS (549 mg, 2.21 mmol, 681 uL), Na₂CO₃ (468. mg, 4.42 mmol) and NiCl₂.dtbbpy (13.1 mg, 33.1 umol). The mixture was stirred at 25° C. for 14 hrs. On completion, the mixture was filtered and concentrated in vacuo to give the residue. The residue was purified by prep-HPLC (column: Phenomenex luna C18 250*50 mm*15 um; mobile phase: [water (FA)-ACN]; B %: 60%-95%, 36 min) to give the title compound (900 mg, 63% yield) as a white solid. ¹H NMR (400 MHz, DMSO-d₆) δ 9.45 (s, 1H), 8.51 (s, 1H), 7.31 (d, J=8.4 Hz, 1H), 7.11 (d, J=2.0 Hz, 1H), 7.07-7.01 (m, 1H), 3.93 (t, J=8.0 Hz, 1H), 3.86 (s, 4H), 3.27-3.23 (m, 2H), 3.20-3.14 (m, 2H), 2.79 (t, J=11.2 Hz, 1H), 2.40-2.33 (m, 2H), 2.17 (s, 3H), 1.98-1.85 (m, 2H), 1.79-1.72 (m, 4H), 1.68 (d, J=12.0 Hz, 2H), 1.59-1.51 (m, 4H), 1.47-1.42 (m, 2H), 1.37 (s, 9H). LC-MS (ESI⁺) m/z 649.3 (M+H)⁺.

### Step 3—4-[2-[4-(7-Azaspiro[3.5]nonan-2-ylsulfanyl)-2-methyl-anilino]-5-(trifluoromethyl)pyrimidin-4-yl]cyclohexanone

To a solution of tert-butyl 2-[4-[[4-(1,4-dioxaspiro[4.5]decan-8-yl)-5-(trifluoromethyl)pyrimidin-2-yl]amino]-3-methyl-phenyl]sulfanyl-7-azaspiro[3.5]nonane-7-carboxylate (900 mg, 1.39 mmol) in HCOOH (2 mL). The mixture was stirred at 70° C. for 1 hr. On completion, the mixture was concentrated in vacuo to give the title compound (700 mg, 100% yield) as yellow oil. LC-MS (ESI⁺) m/z 505.1 (M+H)⁺.

### Step 4—Tert-butyl 2-[3-methyl-4-[[4-(4-oxocyclohexyl)-5-(trifluoromethyl)pyrimidin-2-yl]amino]phenyl]sulfanyl-7-azaspiro[3.5]nonane-7-carboxylate

To a solution of 4-[2-[4-(7-azaspiro[3.5]nonan-2-ylsulfanyl)-2-methyl-anilino]-5-(trifluoromethyl)pyrimidin-4-yl]cyclohexanone (700 mg, 1.39 mmol) in DCM (7 mL) was added TEA (280 mg, 2.77 mmol, 386 uL) and Boc₂O (333 mg, 1.53 mmol, 350 uL). The mixture was stirred at 25° C. for 1 hr. On completion, the mixture was diluted with H₂O (10 mL), extracted with EA (2×5 mL), the organic layers were washed with brine (2×3 mL), dried over anhydrous Na₂SO₄, filtered and concentrated in vacuo to give a residue. The residue was purified by column chromatography (SiO₂, Petroleum ether/Ethyl acetate=100/1 to 50/1) to give the title compound (1.00 g, 77% yield) as a white solid. ¹H NMR (400 MHz, DMSO-d₆) δ 9.48 (s, 1H), 8.58 (s, 1H), 7.31 (d, J=8.4 Hz, 1H), 7.12 (d, J=1.6 Hz, 1H), 7.09-7.01 (m, 1H), 3.93 (t, J=8.2 Hz, 1H), 3.27-3.23 (m, 2H), 3.20-3.14 (m, 2H), 2.64-2.52 (m, 4H), 2.39-2.33 (m, 2H), 2.28 (d, J=14.4 Hz, 2H), 2.17 (s, 3H), 2.02 (s, 2H), 1.94 (d, J=6.8 Hz, 1H), 1.77-1.71 (m, 2H), 1.56-1.52 (m, 2H), 1.46-1.42 (m, 2H), 1.37 (s, 9H). LC-MS (ESI⁺) m/z 605.2 (M+H)⁺.

### Step 5—Tert-butyl 2-((4-((4-((1s,4s)-4-hydroxy-4-methylcyclohexyl)-5-(trifluoromethyl)pyrimidin-2-yl)amino)-3-methylphenyl)thio)-7-azaspiro[3.5]nonane-7-carboxylate and tert-butyl 2-((4-((4-((1r,4r)-4-hydroxy-4-methylcyclohexyl)-5-(trifluoromethyl)pyrimidin-2-yl)amino)-3-methylphenyl)thio)-7-azaspiro[3.5]nonane-7-carboxylate

A mixture of tert-butyl 2-[3-methyl-4-[[4-(4-oxocyclohexyl)-5-(trifluoromethyl)pyrimidin-2-yl]amino]phenyl]sulfanyl-7-azaspiro[3.5]nonane-7-carboxylate (100 mg, 165 umol) in THF (1 mL) was degassed and purged with N₂ for 3 times at −78° C. Then MeLi (1.6 M, 310 uL) was added at −78° C. and the mixture was stirred at −78° C. for 2 hrs under N₂ atmosphere. On completion, the reaction mixture was quenched by addition of NH₄Cl (0.5 mL) at 0° C., and then diluted with H₂O (3 mL) and extracted with EA (5 mL×3). The combined organic layers were washed with brine (3 mL), dried over Na₂SO₄, filtered and concentrated in vacuo to give a residue. The residue was purified by prep-TLC (SiO₂, PE:EA=5:1) to give tert-butyl 2-((4-((4-((1s,4s)-4-hydroxy-4-methylcyclohexyl)-5-(trifluoromethyl)pyrimidin-2-yl)amino)-3-methylphenyl)thio)-7-azaspiro[3.5]nonane-7-carboxylate (100 mg, 75% yield, ¹H NMR (400 MHz, DMSO-d₆) δ 9.42 (s, 1H), 8.50 (s, 1H), 7.34 (d, J=8.0 Hz, 1H), 7.11 (d, J=1.6 Hz, 1H), 7.06-7.01 (m, 1H), 3.98 (s, 1H), 3.96-3.88 (m, 1H), 3.27-3.24 (m, 2H), 3.20-3.16 (m, 2H), 2.67 (t, J=11.2 Hz, 1H), 2.40-2.34 (m, 2H), 2.18 (s, 3H), 2.10-2.01 (m, 2H), 1.78-1.72 (m, 2H), 1.63 (d, J=12.4 Hz, 2H), 1.56-1.52 (m, 2H), 1.47-1.42 (m, 4H), 1.37 (s, 9H), 1.35-1.30 (m, 2H), 1.12 (s, 3H). LC-MS (ESI⁺) m/z 621.2 (M+H)+) and tert-butyl 2-((4-((4-((1r,4r)-4-hydroxy-4-methylcyclohexyl)-5-(trifluoromethyl)pyrimidin-2-yl)amino)-3-methylphenyl)thio)-7-azaspiro[3.5]nonane-7-carboxylate (60 mg, 45% yield, ¹H NMR (400 MHz, DMSO-d₆) δ 9.44 (s, 1H), 8.54 (s, 1H), 7.35 (d, J=8.0 Hz, 1H), 7.12 (s, 1H), 7.07-7.01 (m, 1H), 4.37 (s, 1H), 3.97-3.85 (m, 1H), 3.25 (s, 2H), 3.17 (s, 2H), 2.68 (d, J=4.8 Hz, 1H), 2.39-2.31 (m, 2H), 2.18 (s, 3H), 1.71 (s, 3H), 1.61 (d, J=9.6 Hz, 5H), 1.56-1.51 (m, 2H), 1.43 (d, J=5.6 Hz, 4H), 1.37 (s, 9H), 1.16-1.03 (m, 3H). LC-MS (ESI⁺) m/z 621.2 (M+H)+) as a white solids. Absolute stereochemistry of diastereomers was assigned arbitrarily.

### Synthesis of 4-[2-[4-(7-Azaspiro[3.5]nonan-2-ylsulfonyl)-2-methyl-anilino]-5-(trifluoromethyl)pyrimidin-4-yl]-1-methyl-cyclohexanol (Intermediate MY)

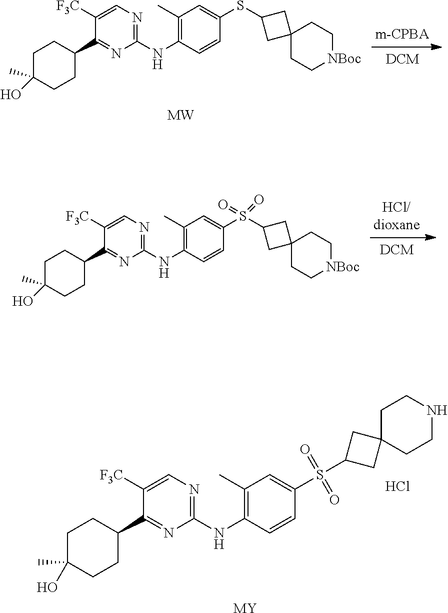

### Step 1—Tert-butyl 2-[4-[[4-(4-hydroxy-4-methyl-cyclohexyl)-5-(trifluoromethyl)pyrimidin-2-yl] amino]-3-methyl-phenyl]sulfonyl-7-azaspiro[3.5]nonane-7-carboxylate

To a solution of tert-butyl 2-[4-[[4-(4-hydroxy-4-methyl-cyclohexyl)-5-(trifluoromethyl) pyrimidin-2-yl]amino]-3-methyl-phenyl]sulfanyl-7-azaspiro[3.5]nonane-7-carboxylate (40.0 mg, 64.4 umol, Intermediate MW) in DCM (1 mL) was added m-CPBA (26.1 mg, 128 umol, 85% solution) at 0° C. The mixture was stirred at 25° C. for 1 hr. On completion, the reaction mixture was quenched by addition of Na₂S₂O₃ (0.5 mL) at 0° C., and then diluted with aq. NaHCO₃ (8 mL) and extracted with DCM (3×5 mL). The combined organic layers were washed with brine (2×3 mL), dried with anhydrous Na₂SO₄, filtered and concentrated in vacuo to give the title compound (40 mg, 65% yield) as a white solid. ¹H NMR (400 MHz, DMSO-d₆) δ 9.69 (s, 1H), 8.63 (s, 1H), 7.89 (d, J=8.4 Hz, 1H), 7.71 (d, J=1.6 Hz, 1H), 7.65-7.60 (m, 1H), 4.17-4.07 (m, 1H), 4.05-4.02 (m, 1H), 3.25-3.19 (m, 6H), 2.36 (s, 3H), 2.09 (d, J=8.4 Hz, 3H), 2.03-1.94 (m, 4H), 1.64 (d, J=12.4 Hz, 2H), 1.48 (d, J=3.6 Hz, 2H), 1.44 (d, J=4.4 Hz, 2H), 1.37 (s, 9H), 1.33 (d, J=3.6 Hz, 1H), 1.24 (s, 1H), 1.13 (s, 3H). LC-MS (ESI⁺) m/z 653.4 (M+H)⁺.

### Step 2—4-[2-[4-(7-Azaspiro[3.5]nonan-2-ylsulfonyl)-2-methyl-anilino]-5-(trifluoromethyl)pyrimidin-4-yl]-1-methyl-cyclohexanol

To a solution of tert-butyl 2-[4-[[4-(4-hydroxy-4-methyl-cyclohexyl)-5-(trifluoromethyl) pyrimidin-2-yl]amino]-3-methyl-phenyl]sulfonyl-7-azaspiro[3.5]nonane-7-carboxylate (50.0 mg, 76.6 umol) in DCM (0.5 mL) was added HCl/dioxane (4 M, 500 uL). The mixture was then stirred at 25° C. for 1 hr. On completion, the mixture was concentrated in vacuo to give the title compound (42.0 mg, 99% yield) as a white solid. LC-MS (ESI⁺) m/z 553.2 (M+H)⁺.

### Synthesis of 1-[1-(2,6-Dioxo-3-piperidyl)-4-fluoro-3-methyl-2-oxo-benzimidazol-5-yl]piperidine-4-carbaldehyde (Intermediate MZ)

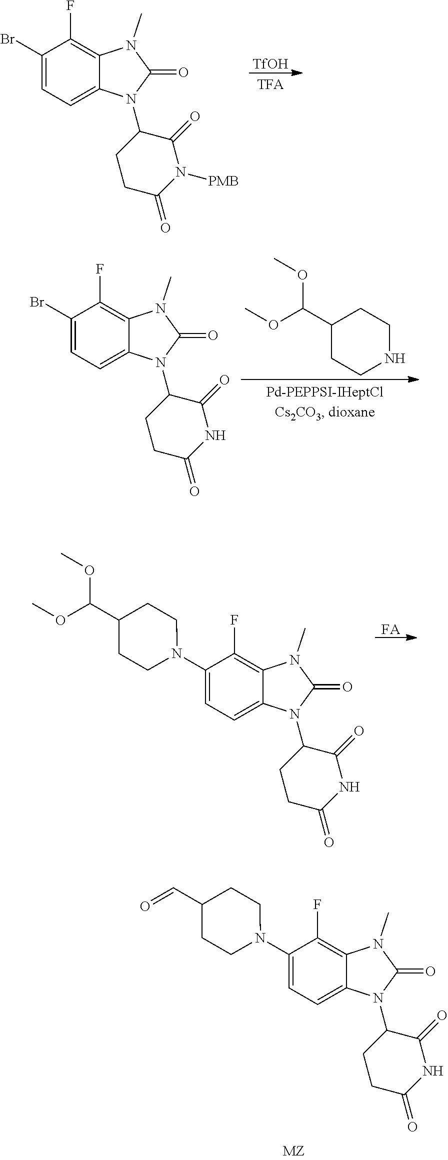

### Step 1—3-(5-Bromo-4-fluoro-3-methyl-2-oxo-benzimidazol-1-yl)piperidine-2,6-dione

To a solution of 3-(5-bromo-4-fluoro-3-methyl-2-oxo-benzimidazol-1-yl)-1-[(4-methoxyphenyl) methyl]piperidine-2,6-dione (5.00 g, 10.5 mmol, synthesized via Steps 1-4 of Intermediate MU) in TFA (40 mL) was added TfOH (13.6 g, 90.6 mmol, 8 mL), then the mixture was stirred at 70° C. for 1 hr. On completion, the mixture was concentrated in vacuo to give the residue. The residue was added TEA to adjust pH=9, triturated with water (100 mL) for 2 hrs and filtered to give the filter cake. Then the filter cake was triturated with PE/EA (1:1, 150 mL) for 3 hrs to give the title compound (1.70 g, 45% yield) as a brown solid. ¹H NMR (400 MHz, DMSO-d₆) δ 11.14 (s, 1H), 7.36-7.26 (m, 1H), 7.05-6.95 (m, 1H), 5.46-5.35 (m, 1H), 3.54-3.44 (m, 3H), 2.94-2.82 (m, 1H), 2.76-2.58 (m, 2H), 2.08-1.98 (m, 1H). LC-MS (ESI⁺) m/z 355.9 (M+H)⁺.

### Step 2—3-[5-[4-(Dimethoxymethyl)-1-piperidyl]-4-fluoro-3-methyl-2-oxo-benzimidazol-1-yl]piperidine-2,6-dione

To a solution of 3-(5-bromo-4-fluoro-3-methyl-2-oxo-benzimidazol-1-yl)piperidine-2,6-dione (3.00 g, 8.42 mmol) and 4-(dimethoxymethyl)piperidine (1.88 g, 11.8 mmol, CAS #188646-83-5) in dioxane (70 mL) was added Pd-PEPPSI-IHeptCl 3-Chloropyridine (819 mg, 842 umol) and Cs₂CO₃ (5.49 g, 16.8 mmol). The mixture was then stirred 100° C. for 4 hrs. On completion, the mixture was filtered and concentrated in vacuo to give the residue. The residue was purified by column chromatography (SiO₂, PE:EA=10:1 to 1:2) to give the title compound (1.30 g, 35% yield). ¹H NMR (400 MHz, DMSO-d₆) δ 11.10 (s, 1H), 6.84 (d, J=8.4 Hz, 1H), 6.77-6.67 (m, 1H), 5.33 (dd, J=5.2, 12.8 Hz, 1H), 4.16-4.08 (m, 1H), 3.46 (d, J=1.6 Hz, 3H), 3.27 (s, 6H), 2.93-2.83 (m, 1H), 2.66-2.56 (m, 4H), 2.03-1.96 (m, 1H), 1.72 (d, J=11.2 Hz, 2H), 1.46-1.37 (m, 2H), 1.21-1.01 (m, 1H), 0.88-0.72 (m, 2H). LC-MS (ESI⁺) m z 435.1 (M+H)⁺.

### Step 3—1-[1-(2,6-Dioxo-3-piperidyl)-4-fluoro-3-methyl-2-oxo-benzimidazol-5-yl]piperidine-4-carbaldehyde

A solution of 3-[5-[4-(dimethoxymethyl)-1-piperidyl]-4-fluoro-3-methyl-2-oxo-benzimidazol -1-yl]piperidine-2,6-dione (100 mg, 230 umol) in FA (1 mL) was stirred at 80° C. for 1 hr. On completion, the mixture was concentrated in vacuo to give the title compound (80 mg, 90% yield) as a black solid.). LC-MS (ESI⁺) m/z 389.0 (M+H)⁺.

### Synthesis of 2-[4-(7-Azaspiro[3.5]nonan-2-ylsulfonyl)-2-methyl-anilino]-8-cyclopentyl-pyrido[2,3-d]pyrimidin-7-one (Intermediate NA)

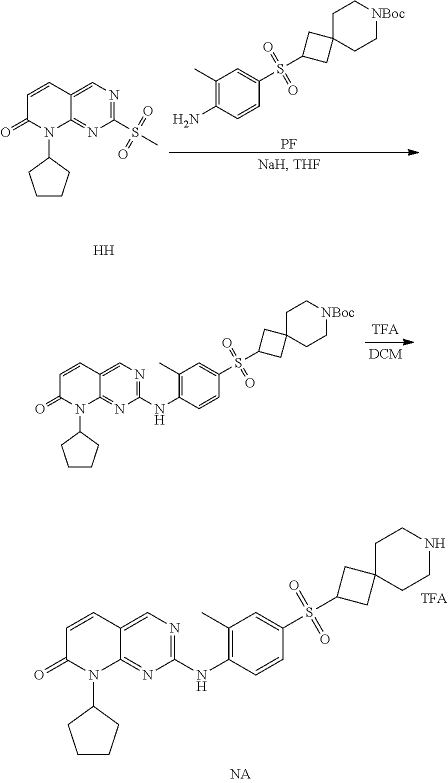

### Step 1—Tert-butyl 2-[4-[(8-cyclopentyl-7-oxo-pyrido[2,3-d]pyrimidin-2-yl)amino]-3-methyl-phenyl]sulfonyl-7-azaspiro[3.5]nonane-7-carboxylate

To a solution of tert-butyl 2-(4-amino-3-methyl-phenyl)sulfonyl-7-azaspiro[3.5]nonane-7-carboxylate (588 mg, 1.49 mmol, Intermediate PF) in DMF (6 mL) was added t-BuOK (668 mg, 5.96 mmol) and 4A molecular sieves (0.1 g). The mixture was stirred at 0° C. for 20 mins. Then to the mixture was added 8-cyclopentyl-2-methylsulfonyl-pyrido[2,3-d]pyrimidin-7-one (480 mg, 1.64 mmol, Intermediate HN) and the mixture was stirred at 0° C. for 40 mins. On completion, the reaction mixture was diluted with H₂O (5 mL) and extracted with EA (3×50 mL). The combined organic layers were washed with brine (2×10 mL), dried over by anhydrous Na₂SO₄, filtered and concentrated under reduced pressure to give a residue. Then the residue was purified by column chromatography (SiO₂, Petroleum ether/Ethyl acetate=100/1 to 2/1) to give the title compound (327 mg, 32% yield) as a white solid. LC-MS (ESI⁺) m z 608.3 (M+H)⁺.

### Step 2—2-[4-(7-Azaspiro[3.5]nonan-2-ylsulfonyl)-2-methyl-anilino]-8-cyclopentyl-pyrido[2,3-d]pyrimidin-7-one

To a solution of tert-butyl 2-[4-[(8-cyclopentyl-7-oxo-pyrido[2,3-d]pyrimidin-2-yl)amino]-3-methyl -phenyl]sulfonyl-7-azaspiro[3.5]nonane-7-carboxylate (130 mg, 213 umol) in DCM (1 mL) was added TFA (1.54 g, 13 mmol). The mixture was then stirred at 25° C. for 1 hr. On completion, the reaction mixture was concentrated in vacuo to give a title compound (100 mg, 92% yield, TFA salt) as a white solid. LC-MS (ESI⁺) m/z 508.2 (M+H)⁺.

### Tert-butyl 2-[4-[[4-chloro-5-(trifluoromethyl)pyrimidin-2-yl]amino]-3-methyl-phenyl] sulfonyl-7-azaspiro[3.5]nonane-7-carboxylate (Intermediate NB)

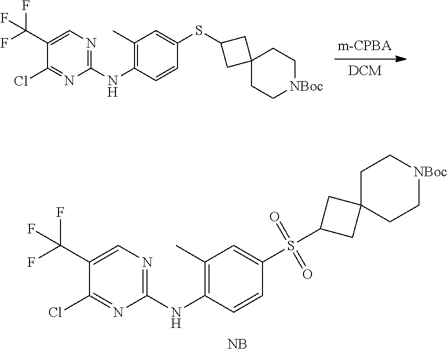

To a solution of tert-butyl 2-[4-[[4-chloro-5-(trifluoromethyl)pyrimidin-2-yl]amino]-3-methyl-phenyl] sulfanyl-7-azaspiro[3.5]nonane-7-carboxylate (356 mg, 655 umol, synthesized via Step 1 of Intermediate MW) in DCM (4 mL) was added m-CPBA (424 mg, 1.97 mmol). The mixture was then stirred at 25° C. for 2 hrs. On completion, the reaction mixture was quenched by addition of Na₂S₂O₃ (3 mL) at 0° C., and then diluted with H₂O (5 mL) and extracted with EA (3×3 mL). The combined organic layers were washed with brine (3×2 mL), dried over by anhydrous Na₂SO₄, filtered and concentrated in vacuo to give a residue. The residue was purified by column chromatography (SiO₂, Petroleum ether/Ethyl acetate=100/1 to 1/1) to give the title compound (210 mg, 45% yield) as a yellow solid. LC-MS (ESI⁺) m z 597.2 (M+23)+. ¹HNMR (400 MHz, DMSO-d₆) δ 10.35 (s, 1H), 8.77 (s, 1H), 7.77-7.72 (m, 2H), 7.71-7.67 (m, 1H), 4.21-4.12 (m, 1H), 3.25-3.18 (m, 4H), 2.34 (s, 3H), 2.15-2.07 (m, 2H), 2.02-1.94 (m, 2H), 1.52-1.48 (m, 2H), 1.46-1.42 (m, 2H), 1.38 (s, 9H).

### Synthesis of 1-[2-[4-(7-Azaspiro[3.5]nonan-2-ylsulfonyl)-2-methyl-anilino]-5-(trifluoromethyl)pyrimidin -4-yl]-3-methyl-piperidin-3-ol (Intermediate NC)

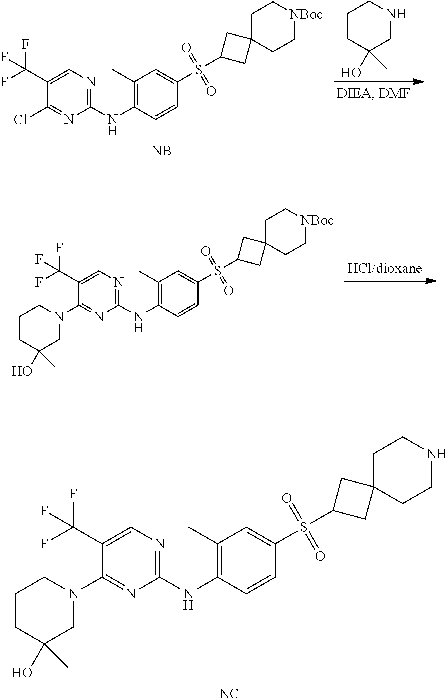

### Step 1—Tert-butyl 2-[4-[[4-(3-hydroxy-3-methyl-1-piperidyl)-5-(trifluoromethyl)pyrimidin-2-yl]amino]-3-methyl-phenyl]sulfonyl-7-azaspiro[3.5]nonane-7-carboxylate

To a solution of tert-butyl 2-[4-[[4-chloro-5-(trifluoromethyl)pyrimidin-2-yl]amino]-3-methyl-phenyl] sulfonyl-7-azaspiro[3.5]nonane-7-carboxylate (190 mg, 330 umol, Intermediate NB) in DMF (2 mL) was added DIEA (85.4 mg, 660 umol, 115 uL) and 3-methylpiperidin-3-ol (45.6 mg, 396 umol, CAS #473730-88-0). The mixture was then stirred at 25° C. for 2 hrs. On completion, the reaction mixture was diluted with H₂O (4 Ml) and extracted with EA (3×15 mL). The combined organic layers were dried over by anhydrous Na₂SO₄, filtered and concentrated in vacuo to give the title compound (200 mg, 89% yield) as a white solid. LC-MS (ESI⁺) m/z 654.3 (M+1). ¹H NMR (400 MHz, DMSO-d₆) δ 9.14 (s, 1H), 8.38-8.34 (m, 1H), 7.98 (s, 1H), 7.72-7.67 (m, 1H), 7.65-7.59 (m, 1H), 4.12-4.06 (m, 1H), 3.28 (d, J=13.2 Hz, 2H), 3.23-3.17 (m, 4H), 3.15-3.11 (m, 2H), 2.35 (s, 3H), 2.12-2.05 (m, 2H), 1.98 (d, J=8.8 Hz, 2H), 1.82-1.73 (m, 1H), 1.59-1.54 (m, 2H), 1.49-1.47 (m, 2H), 1.44-1.39 (m, 4H), 1.37 (s, 9H), 1.04 (s, 3H).

### Step 2—1-[2-[4-(7-Azaspiro[3.5]nonan-2-ylsulfonyl)-2-methyl-anilino]-5-(trifluoromethyl)pyrimidin -4-yl]-3-methyl-piperidin-3-ol

To a solution of tert-butyl 2-[4-[[4-(3-hydroxy-3-methyl-1-piperidyl)-5-(trifluoromethyl) pyrimidin-2-yl]amino]-3-methyl-phenyl]sulfonyl-7-azaspiro[3.5]nonane-7-carboxylate (100 mg, 152 umol) in DCM (0.5 mL) was added HCl/dioxane (0.5 mL). The mixture was then stirred at 25° C. for 1 hr. On completion, the reaction mixture was concentrated in vacuo to give the title compound (80 mg, 94% yield) as a yellow solid. LC-MS (ESI⁺) m/z 554.3 (M+H)⁺.

### Synthesis of 4-[[4-(4-hydroxy-4-methyl-cyclohexyl)-5-(trifluoromethyl)pyrimidin-2-yl] amino]-3-methyl-benzenesulfonyl chloride (Intermediate ND)

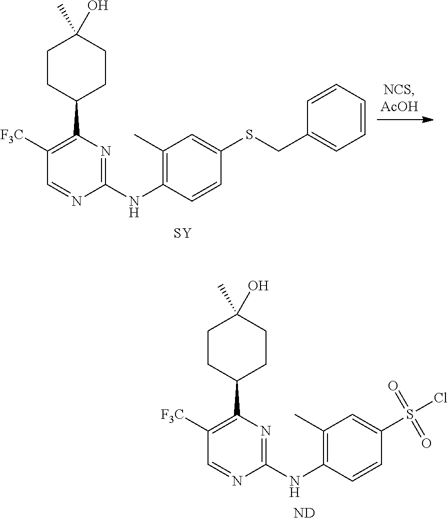

To a solution of (1r,4r)-4-(2-((4-(benzylthio)-2-methylphenyl)amino)-5-(trifluoromethyl)pyrimidin-4-yl)-1-methylcyclohexan-1-ol (55.0 mg, 112 umol, Intermediate SY) in CH₃CN (2 mL), H₂O (0.1 mL) and HOAc (0.1 mL) was added NCS (45.1 mg, 338 umol). The mixture was then stirred at 25° C. for 1 hr. On completion, the reaction mixture diluted with DCM (10 mL), the reaction mixture was dried over Na₂SO₄, filtered and concentrated in vacuo to give the title compound (50.0 mg, 95% yield) as white solid. LCMS (ESI⁺) m/z 463.9 (M+H)⁺.

### Synthesis of 3-[4-Fluoro-3-methyl-2-oxo-5-(4-piperidyl)benzimidazol-1-yl]piperidine-2,6-dione (Intermediate NE)

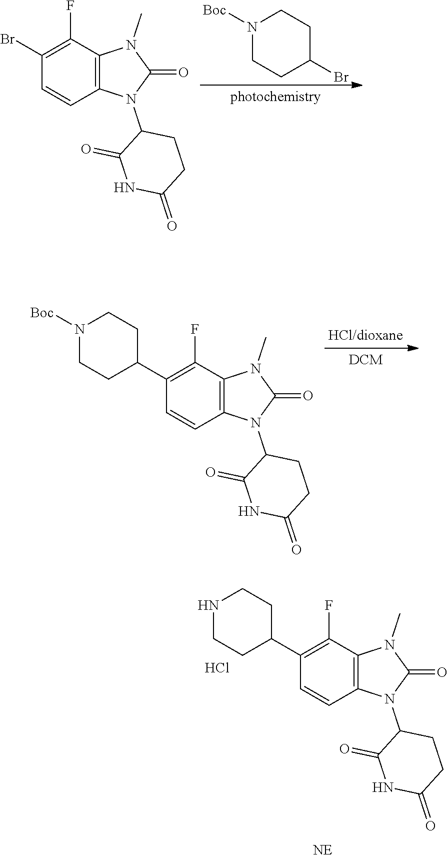 [SMILES: Cn1c(=O)n(C2CCC(=O)NC2=O)c2ccc(Br)c(F)c21 ; CC(C)(C)OC(=O)N1CCC(Br)CC1 ; Cn1c(=O)n(C2CCC(=O)NC2=O)c2ccc(C3CCN(C(=O)OC(C)(C)C)CC3)c(F)c21 ; Cl ; Cn1c(=O)n(C2CCC(=O)NC2=O)c2ccc(C3CCNCC3)c(F)c21]

### Step 1—Tert-butyl 4-[1-(2,6-dioxo-3-piperidyl)-4-fluoro-3-methyl-2-oxo-benzimidazol-5-yl]piperidine-1-carboxylate

To an 15 mL vial equipped with a stir bar was added 3-(5-bromo-4-fluoro-3-methyl-2-oxo-benzimid azol-1-yl)piperidine-2,6-dione (800 mg, 2.25 mmol, synthesized via Step 1 of Intermediate MZ) tert-butyl4-bromopiperidine-1-carboxylate (771 mg, 2.92 mmol, CAS #180695-79-8), Ir[dF(CF₃)ppy]₂(dtbpy)(PF₆) (25.2 mg, 22.4 umol), NiCl₂.dtbbpy (13.4 mg, 33.6 umol), TTMSS (558 mg, 2.25 mmol), and 2,6-lutidine (481 mg, 4.49 mmol) in DME (2 mL). The vial was sealed and placed under nitrogen was added. The reaction was stirred and irradiated with a purple 10 W LED lamp (3 cm away), with cooling water to keep the reaction temperature at 25° C. for 14 hrs. The reaction mixture was partitioned between H₂O (30 mL) and EA (30 mL). The organic phase was separated, washed with brine (15 mL), dried over anhydrous Na₂SO₄, filtered and concentrated in vacuo to give a residue. The residue was purified by prep-HPLC (column: Phenomenex luna C18 150*25 mm*10 um; mobile phase: [water(FA)-ACN];B %:34%-64%, 10 min) to give the title compound (850 mg, 82% yield) as a white solid. ¹H NMR (400 MHz, DMSO-d₆) δ ppm 11.10 (s, 1H), 6.97-6.90 (m, 2H), 5.36 (dd, J=12.0, 5.2 Hz, 1H), 4.08 (d, J=10.0 Hz, 2H), 3.48 (d, J=1.2 Hz, 3H), 3.04-2.97 (m, 1H), 2.92-2.80 (m, 3H), 2.74-2.58 (m, 3H), 2.04-1.97 (m, 1H), 1.72 (d, J=12.0 Hz, 2H), 1.60-1.53 (m, 2H), 1.42 (s, 9H). LC-MS (ESI⁺) m/z 483.2 (M+23)+.

### Step 2—3-[4-Fluoro-3-methyl-2-oxo-5-(4-piperidyl)benzimidazol-1-yl]piperidine-2,6-dione

To a solution of tert-butyl 4-[1-(2,6-dioxo-3-piperidyl)-4-fluoro-3-methyl-2-oxo-benzimidazol-5-yl] piperidine-1-carboxylate (110 mg, 238 umol) in DCM (1 mL) was added HCl/dioxane (4 M, 1 mL). The mixture was then stirred at 25° C. for 1 hr. On completion, the reaction mixture was concentrated in vacuo to give the title compound (90.0 mg, 94% yield, HCl) as a white solid. LC-MS (ESI⁺) m/z 361.1 (M+H)⁺.

### Synthesis of Tert-butyl N-[2-(4-formylcyclohexoxy)ethyl]carbamate (Intermediate NF)

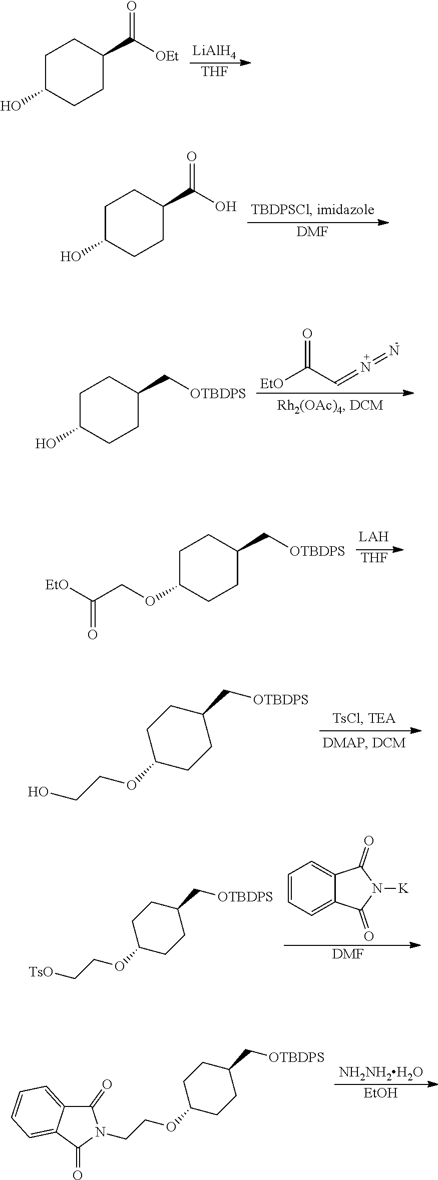

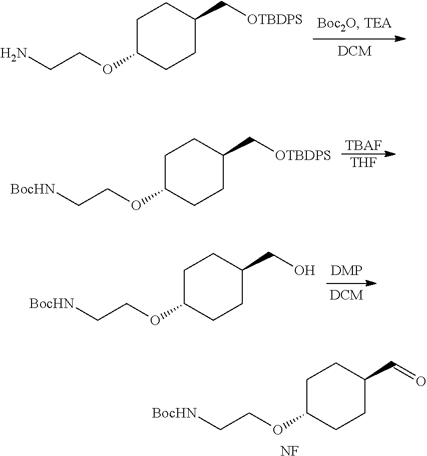

### Step 1—4-(Hydroxymethyl)cyclohexanol

To a solution of LiAlIH₄ (3.31 g, 87.1 mmol) in THF (30 mL), was add ethyl 4-hydroxycyclohexanecarboxylate (10.0 g, 58.0 mmol, CAS #3618-04-0) in THF (100 mL) dropwise at 0° C., then the mixture was stirred at 0° C. for 5 hrs. On completion, the mixture was quenched with H₂O (3.3 mL), then a solution of 15% NaOH (3.3 mL) was added dropwise. The mixture was dried with anhydrous Na₂SO₄, filtered and the filtered liquor was concentrated in vacuo to give the title compound (7.5 g, 99% yield) as yellow solid. ¹H NMR (400 MHz, DMSO-d₆) δ 3.37-3.23 (m, 1H), 3.17 (d, J=6.0 Hz, 2H), 1.85-1.75 (m, 2H), 1.75-1.62 (m, 2H), 1.30-1.16 (m, 1H), 1.14-0.95 (m, 2H), 0.93-0.72 (m, 2H).

### Step 2-4-[[Tert-butyl(diphenyl)silyl]oxymethyl]cyclohexanol

To a solution of 4-(hydroxymethyl)cyclohexanol (6.5 g, 49.9 mmol) and imidazole (4.08 g, 59.9 mmol) in DMF (200 mL) was added TBDPSCl (14.4 g, 52.4 mmol) at 0° C. The mixture was stirred at 25° C. for 12 hrs. On completion, the mixture was diluted with H₂O (100 mL), and extracted with EA (2×30 mL). The organic layers were washed with brine (2×30 mL), dried over anhydrous Na₂SO₄, filtered and concentrated in vacuo. The mixture was purified by silica gel column (PE:EA=5:1) to give the title compound (9.10 g, 49% yield) as yellow oil. ¹H NMR (400 MHz, CDCl₃) δ 7.70-7.60 (m, 4H), 7.48-7.31 (m, 6H), 3.63-3.51 (m, 1H), 3.47 (d, J=6.0 Hz, 2H), 2.05-1.95 (m, 2H), 1.89-1.80 (m, 2H), 1.50-1.45 (m, 1H), 1.31-1.22 (m, 2H), 1.10-1.00 (m, 2H), 1.05 (s, 9H).

### Step 3—Ethyl 2-[4-[[tert-butyl(diphenyl)silyl]oxymethyl]cyclohexoxy]acetate

To a solution of 4-[[tert-butyl (diphenyl)silyl]oxymethyl]cyclohexanol (8.60 g, 23.3 mmol) and Rh₂(OAc)₄ (1.03 g, 2.33 mmol) in DCM (40 mL) was added a solution of ethyl 2-diazoacetate (10.6 g, 93.3 mmol) in DCM (40 mL). The mixture was degassed and purged with N₂ for 3 times and the mixture was stirred at 25° C. for 12 hrs under N₂ atmosphere. On completion, the mixture was diluted with DCM (80 mL), the organic layers was then separated, washed with H₂O (2×80 mL), brine (2×80 mL), dried over anhydrous Na₂SO₄, filtered and concentrated in vacuo. The mixture was purified by silica gel column (PE:EA=10:1) to give the title compound (10.0 g, 94% yield) as yellow oil. ¹H NMR (400 MHz, CDCl₃) δ 7.72-7.61 (m, 4H), 7.48-7.32 (m, 6H), 4.28-4.25 (m, 2H), 4.12 (s, 2H), 3.46 (d, J=6.0 Hz, 2H), 3.32-3.22 (m, 1H), 2.15-2.04 (m, 2H), 1.91-1.81 (m, 2H), 1.54-1.45 (m, 1H), 1.32-1.28 (m, 5H), 1.09-0.99 (m, 2H), 1.05 (s, 9H).

### Step 4—2-[4-[[Tert-butyl(diphenyl)silyl]oxymethyl]cyclohexoxy]ethanol

To a solution of LAH (626 mg, 16.5 mmol) in THF (25 mL) was added a solution of ethyl 2-[4-[[tertbutyl(diphenyl)silyl]oxymethyl]cyclohexoxy]acetate (5.00 g, 11.0 mmol) in THF (25 mL) dropwise at 0° C. The mixture was then stirred at 0° C. for 0.5 hr. On completion, the mixture was quenched with H₂O (0.62 mL), then a solution of 15% NaOH (0.62 mL) was added dropwise. The mixture was dried with anhydrous Na₂SO₄, filtered and the filtered liquor was concentrated in vacuo to give the title compound (3.15 g, 69% yield) as yellow oil. ¹H NMR (400 MHz, CDCl₃) δ 7.76-7.61 (m, 4H), 7.49-7.33 (m, 6H), 3.81-3.66 (m, 3H), 3.63-3.57 (m, 2H), 3.50-3.45 (m, 2H), 3.28-3.18 (m, 1H), 2.15-2.04 (m, 2H), 1.88-1.83 (m, 2H), 1.61-1.46 (m, 1H), 1.28-1.20 (m, 2H), 1.10-0.96 (m, 2H), 1.05 (s, 9H).

### Step 5—2-[4-[[Tert-butyl(diphenyl)silyl]oxymethyl]cyclohexoxy]ethyl 4-methylbenzenesulfonate

To a solution of 2-[4-[[tert-butyl(diphenyl)silyl]oxymethyl]cyclohexoxy]ethanol (3.15 g, 7.63 mmol) in DCM (40 mL) was added TEA (1.13 g, 11.1 mmol), DMAP (170 mg, 1.39 mmol) and TosCl (1.59 g, 8.35 mmol). The mixture was then stirred at 25° C. for 16 hrs. On completion, the mixture was concentrated in vacuo. The mixture was purified by silica gel column (PE:EA=10:1) to give the title compound (2.86 g, 90% yield) as colorless oil. ¹H NMR (400 MHz, CDCl₃) δ 7.88-7.76 (m, 2H), 7.68-7.60 (m, 4H), 7.44-7.31 (m, 8H), 4.19-4.12 (m, 2H), 3.72-3.62 (m, 2H), 3.45 (d, J=6.4 Hz, 2H), 3.19-3.06 (m, 1H), 2.45 (s, 3H), 2.00-1.90 (m, 2H), 1.88-1.75 (m, 2H), 1.52-1.42 (m, 1H), 1.20-1.10 (m, 2H), 1.05 (s, 9H), 1.01-0.92 (m, 2H).

### Step 6—2-[2-[4-[[Tert-butyl(diphenyl)silyl]oxymethyl]cyclohexoxy]ethyl]isoindoline-1,3-dione

To a solution of 2-[4-[[tert-butyl(diphenyl)silyl]oxymethyl]cyclohexoxy]ethyl 4-methylbenzenesulfonate (2.86 g, 5.05 mmol) in DMF (20 mL) was added (1,3-dioxoisoindolin-2-yl) potassium (1.40 g, 7.57 mmol). The mixture was then stirred at 50° C. for 5 hrs. On completion, the mixture was diluted with H₂O (150 mL), extracted with EA (3×50 mL), the organic layers were washed with brine (3×40 mL), dried over anhydrous Na₂SO₄, filtered and concentrated in vacuo to give the title compound (2.7 g, 98% yield) as yellow oil. ¹H NMR (400 MHz, CDCl₃) δ 7.89-7.83 (m, 2H), 7.74-7.69 (m, 2H), 7.68-7.61 (m, 4H), 7.45-7.34 (m, 6H), 3.95-3.82 (m, 2H), 3.77-3.68 (m, 2H), 3.44 (d, J=6.1 Hz, 2H), 3.28-3.15 (m, 1H), 2.03-1.94 (m, 2H), 1.87-1.75 (m, 2H), 1.54-1.40 (m, 1H), 1.22-1.12 (m, 2H), 1.04 (s, 9H), 1.02-0.90 (m, 2H).

### Step 7—2-[4-[[Tert-butyl(diphenyl)silyl]oxymethyl]cyclohexoxy]ethanamine

To a solution of 2-[2-[4-[[tert-butyl(diphenyl)silyl]oxymethyl]cyclohexoxy]ethyl] isoindoline -1,3-dione (2.7 g, 4.98 mmol) in EtOH (20 mL) was added NH₂NH₂·H₂O (3.19 g, 54.1 mmol, 3.10 mL, 85% solution). The mixture was stirred at 50° C. for 2 hrs. On completion, the mixture was filtered and the filtrate was concentrated in vacuo. The residue was diluted with DCM (30 mL), and filtered, the filtrate was concentrated in vacuo to give the title compound (2.02 g, 98% yield) as yellow oil. ¹H NMR (400 MHz, CDCl₃) δ 7.70-7.63 (m, 4H), 7.47-7.35 (m, 6H), 3.54 (t, J=4.8 Hz, 2H), 3.47 (d, J=6.0 Hz, 2H), 3.25-3.15 (m, 1H), 2.90 (t, J=5.2 Hz, 2H), 2.33-2.19 (m, 2H), 2.13-2.00 (m, 2H), 1.88-1.78 (m, 2H), 1.56-1.45 (m, 1H), 1.28-1.20 (m, 2H), 1.06 (s, 9H), 1.04-0.94 (m, 2H).

### Step 8—Tert-butyl N-[2-[4-[[tert-butyl(diphenyl)silyl]oxymethyl]cyclohexoxy]ethyl]carbamate

To a solution of 2-[4-[[tert-butyl(diphenyl)silyl]oxymethyl]cyclohexoxy]ethanamine (2 g, 4.86 mmol) in DCM (20 mL) was added TEA (983 mg, 9.72 mmol, 1.35 mL) and (Boc)₂O (1.27 g, 5.83 mmol, 1.34 mL). The mixture was then stirred at 25° C. for 3 hrs. On completion, the mixture was concentrated in vacuo. The mixture was purified by silica gel column (PE:EA=10:1) to give the title compound (1.88 g, 75% yield) as yellow oil. ¹H NMR (400 MHz, CDCl₃) δ 7.77-7.58 (m, 4H), 7.53-7.32 (m, 6H), 5.00-4.79 (m, 1H), 3.53 (t, J=5.2 Hz, 2H), 3.47 (d, J=6.0 Hz, 2H), 3.33-3.25 (m, 2H), 3.22-3.12 (m, 1H), 2.10-1.99 (m, 2H), 1.89-1.80 (m, 2H), 1.54-1.46 (m, 1H), 1.49 (s, 9H), 1.27-1.15 (m, 2H), 1.06 (s, 9H), 1.05-0.93 (m, 2H).

### Step 9—Tert-butyl N-[2-[4-(hydroxymethyl)cyclohexoxy]ethyl]carbamate

To a solution of tert-butyl N-[2-[4-[[tert-butyl(diphenyl)silyl]oxymethyl]cyclohexoxy]ethyl]carbamate (1.78 g, 3.48 mmol) in THF (15 mL) was added TBAF (1.00 M, 5.22 mL). The mixture was then stirred at 25° C. for 1 hr. On completion, the mixture was concentrated in vacuo. The mixture was purified by silica gel column (PE:EA=1:1) to give the title compound (950 mg, 99% yield) as yellow oil. ¹H NMR (400 MHz, CDCl₃) δ 5.00-4.75 (m, 1H), 3.53 (t, J=5.2 Hz, 2H), 3.47 (d, J=6.4 Hz, 2H), 3.34-3.26 (m, 2H), 3.25-3.13 (m, 1H), 2.13-2.03 (m, 2H), 1.90-1.80 (m, 2H), 1.56-1.50 (m, 1H), 1.46 (s, 9H), 1.30-1.16 (m, 3H), 1.05-0.91 (m, 2H).

### Step 10—Tert-butyl N-[2-(4-formylcyclohexoxy)ethyl]carbamate

To a solution tert-butyl N-[2-[4-(hydroxymethyl)cyclohexoxy]ethyl]carbamate (800 mg, 2.93 mmol) in DCM (20 mL) was added DMP (1.49 g, 3.51 mmol). The mixture was then stirred at 25° C. for 0.5 hr. On completion, the mixture was diluted with DCM (100 mL) and quenched with saturated Na₂S₂O₃ (50 mL) and washed with saturated NaHCO₃ (3×50 mL). The organic layer was dried over anhydrous Na₂SO₄, filtered and concentrated in vacuo to give the title compound (790 mg, 99% yield) as yellow oil. H NMR (400 MHz, CDCl₃) δ 9.65 (d, J=1.2 Hz, 1H), 5.05-4.67 (m, 1H), 3.57-3.49 (m, 2H), 3.37-3.17 (m, 3H), 2.17-1.99 (m, 4H), 1.46 (s, 10H), 1.41-1.23 (m, 4H).

### Synthesis of 3-[5-[1-[[4-(2-Aminoethoxy)cyclohexyl]methyl]-4-piperidyl]-4-fluoro-3-methyl-2-oxo-benzimidazol-1-yl]piperidine-2,6-dione (Intermediate NG)

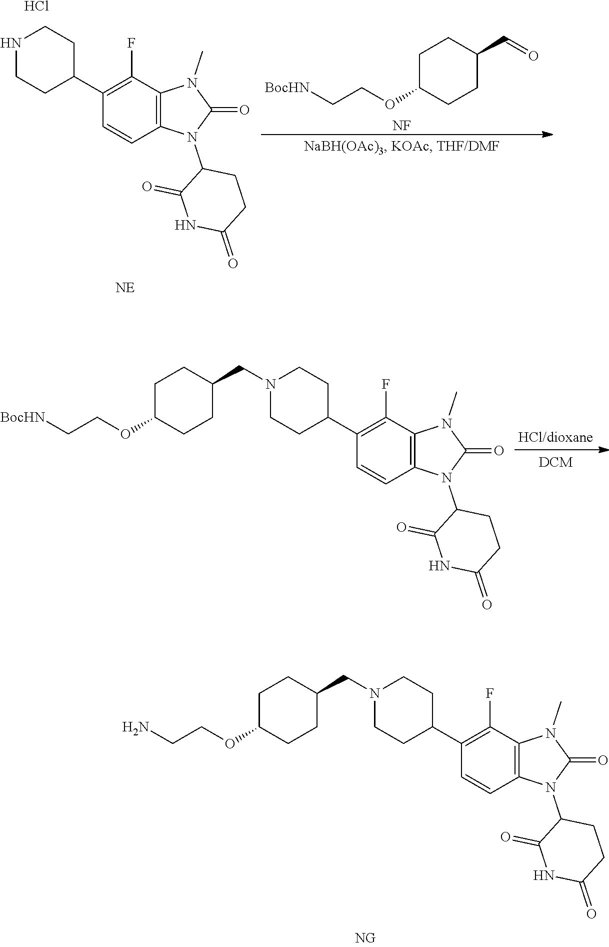

### Step 1—Tert-butyl N-[2-[4-[[4-[1-(2,6-dioxo-3-piperidyl)-4-fluoro-3-methyl-2-oxo-benzimidazol-5-yl]-1-piperidyl]methyl]cyclohexoxy]ethyl]carbamate

To a solution of 3-[4-fluoro-3-methyl-2-oxo-5-(4-piperidyl)benzimidazol-1-yl]piperidine-2,6-dione (90.0 mg, 226 umol, HCl, Intermediate NE) and tert-butyl N-[2-(4-formylcyclohexoxy)ethyl]carbamate (61.5 mg, 226 umol, Intermediate NF) in DMF (1 mL) and THF (1 mL) was added KOAc (222 mg, 2.27 mmol) and NaBH(OAc)₃ (96.1 mg, 453 umol). The mixture was stirred at 25° C. for 1 hr. On completion, the reaction mixture was partitioned between H₂O (20 mL) and DCM (10 mL×3). The organic phase was separated, washed with brine (10 mL×2), dried over anhydrous Na₂SO₄, filtered and concentrated in vacuo to give a residue. The residue was purified by prep-HPLC (column: Phenomenex luna C18 150*25 mm*10 um; mobile phase: [water(FA)-ACN]; B %: 12%-42%, 8 min) to give the title compound (80.0 mg, 57% yield) as a white solid. ¹H NMR (400 MHz, DMSO-d₆) δ 11.10 (s, 1H), 6.98-6.90 (m, 2H), 6.72 (br t, J=5.2 Hz, 1H), 5.35 (dd, J=5.2, 12.8 Hz, 1H), 3.48 (d, J=1.2 Hz, 3H), 3.37 (t, J=6.4 Hz, 2H), 3.18-3.12 (m, 1H), 3.02 (q, J=6.0 Hz, 2H), 2.96 (d, J=11.2 Hz, 2H), 2.91-2.84 (m, 1H), 2.83-2.76 (m, 1H), 2.74-2.66 (m, 1H), 2.65-2.58 (m, 1H), 2.57-2.51 (m, 1H), 2.15 (br d, J=7.2 Hz, 2H), 2.07-1.93 (m, 5H), 1.82-1.74 (m, 3H), 1.72 (s, 2H), 1.45 (dd, J=3.2, 7.6 Hz, 1H), 1.37 (s, 9H), 1.15-1.05 (m, 2H), 0.91-0.80 (m, 2H). LC-MS (ESI⁺) m/z 616.4 (M+H)⁺.

### Step 2—3-[5-[1-[[4-(2-Aminoethoxy)cyclohexyl]methyl]-4-piperidyl]-4-fluoro-3-methyl-2-oxo-benzimidazol-1-yl]piperidine-2,6-dione

To a solution of tert-butyl N-[2-[4-[[4-[1-(2,6-dioxo-3-piperidyl)-4-fluoro-3-methyl-2-oxo-benzimidaz ol-5-yl]-1-piperidyl]methyl]cyclohexoxy]ethyl]carbamate (60.0 mg, 97.4 μmol) in DCM (0.5 mL) was added HCl/dioxane (4 M, 0.5 mL). The mixture was then stirred at 25° C. for 1 hr. On completion, the reaction mixture was concentrated in vacuo to give the title compound (53.0 mg, 98% yield, HCl) as white gum. LCMS (ESI⁺) m/z 516.5 (M+H)⁺.

### Synthesis of 7-Chloro-1-cyclopentyl-1,6-naphthyridin-2(1H)-one (Intermediate NH)

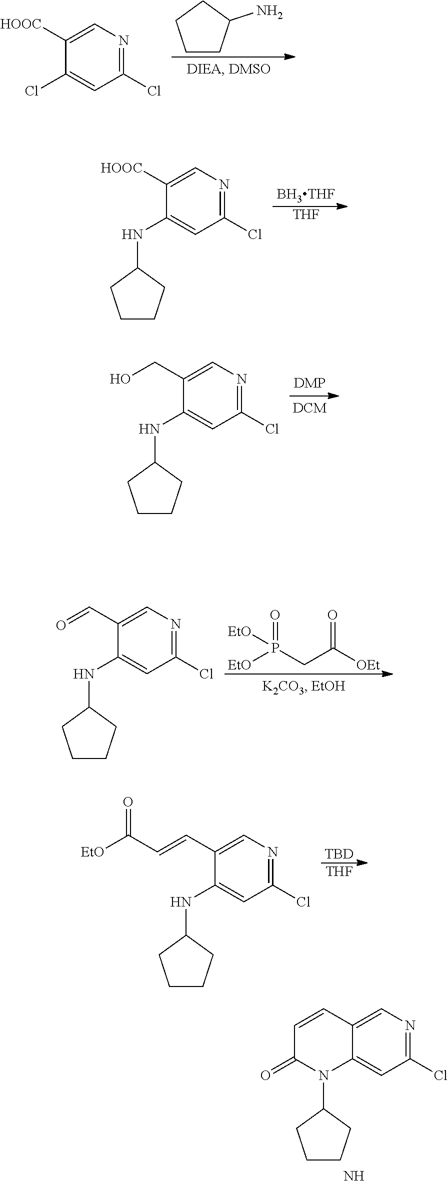

### Step 1—6-Chloro-4-(cyclopentylamino)pyridine-3-carboxylic acid

To a solution of 4,6-dichloropyridine-3-carboxylic acid (2.00 g, 10.4 mmol, CAS #73027-79-9) in DMSO (20 mL) was added DIEA (4.04 g, 31.2 mmol) and cyclopentanamine (1.77 g, 20.8 mmol, CAS#1003-03-8), then the mixture was stirred at 100° C. for 12 hrs. On completion, the reaction mixture was quenched by addition of FA until pH=5 to give the residue. The residue was purified by HPLC (FA condition) to give the title compound (1.65 g, 65% yield) as a yellow solid. ¹H NMR (400 MHz, DMSO-d₆) δ=8.50 (s, 1H), 8.34 (d, J=6.8 Hz, 1H), 6.78 (s, 1H), 4.00-3.91 (m, 1H), 2.08-1.97 (m, 2H), 1.73-1.56 (m, 4H), 1.44 (dd, J=6.0, 12.0 Hz, 2H). LCMS (ESI⁺) m/z 241.2 (M+H)⁺.

### Step 2—(6-Chloro-4-(cyclopentylamino)pyridin-3-yl)methanol

To a solution of 6-chloro-4-(cyclopentylamino)nicotinic acid (1.65 g, 6.86 mmol) in THF (17 mL) was added BH₃·THF (1 M, 20 mL), then the mixture was stirred at 25° C. for 16 hrs. On completion, the reaction mixture was quenched by addition of CH₃OH (10 mL) at 0° C. and FA (0.5 mL) at 25° C., then the mixture was stirred at 25° C. for 16 hrs. Next, NaHCO₃ (800 mg) was added at 25° C. for 10 mins. Then mixture was filtered and the organic phase was concentrated in vacuo to give the crude product was purified by reversed-phase HPLC (FA condition) to give the title compound (1.33 g, 85% yield) as a white solid. ¹H NMR (400 MHz, DMSO-d₆) δ=7.78 (s, 1H), 6.57 (s, 1H), 6.05 (d, J=6.4 Hz, 1H), 4.40 (s, 2H), 3.89-3.83 (m, 1H), 2.00-1.93 (m, 2H), 1.72-1.43 (m, 7H). LCMS (ESI⁺) m/z 227.2 (M+H)⁺.

### Step 3—6-Chloro-4-(cyclopentylamino)nicotinaldehyde

To a solution of [6-chloro-4-(cyclopentylamino)-3-pyridyl]methanol (1.33 g, 5.88 mmol) in DCM (13 mL) was added DMP (3.24 g, 7.64 mmol), then the mixture was stirred at 25° C. for an hr. On completion, the reaction mixture was quenched by Na₂S₂O₃ (aq) (30 mL) and NaHCO₃ (30 mL) at 25° C., and then extracted with DCM (50 mL×3). The combined organic phase was washed with NaCl(aq) (30 mL), dried over Na₂SO₄, filtered and concentrated in vacuo to give the title compound (1.18 g, 89% yield) as a yellow solid. LCMS (ESI⁺) m/z 225.2 (M+H)⁺. Step 4—Ethyl (E)-3-(6-chloro-4-(cyclopentylamino)pyridin-3-yl)acrylate

A mixture of 6-chloro-4-(cyclopentylamino)pyridine-3-carbaldehyde (1.18 g, 5.25 mmol), ethyl 2-diethoxyphosphorylacetate (3.53 g, 15.7 mmol, CAS #867-13-0), K₂CO₃ (1.45 g, 10.5 mmol) in EtOH (13 mL) was degassed and purged with N₂ for 3 times, and then the mixture was stirred at 25° C. for 16 hrs under N₂ atmosphere. The residue was diluted with H₂O (50 mL) and extracted with DCM (40 mL×3). The combined organic layers were washed with NaCl(aq) (50 mL), dried over Na₂SO₄, filtered and concentrated under reduced pressure to give the residue. The residue was purified by column chromatography (SiO₂, Petroleum ether/Ethyl acetate=1/0 to 30/1, Rf=0.54) to give the title compound (0.63 g, 40% yield) as a yellow oil liquid. ¹H NMR (400 MHz, DMSO-d₆) δ=8.22 (s, 1H), 7.88-7.81 (m, 1H), 6.89-6.84 (m, 1H), 6.62 (s, 1H), 6.52-6.46 (m, 1H), 4.19 (q, J=7.2 Hz, 2H), 3.89-3.82 (m, 1H), 1.99-1.91 (m, 2H), 1.71-1.64 (m, 2H), 1.60-1.52 (m, 4H), 1.25 (t, J=7.2 Hz, 3H). LCMS (ESI⁺) m z 295.0 (M+H)⁺.

### Step 5—7-Chloro-1-cyclopentyl-1,6-naphthyridin-2(1H)-one

To a solution of ethyl (E)-3-(6-chloro-4-(cyclopentylamino)pyridin-3-yl)acrylate (630 mg, 2.14 mmol) in THF (20 mL) was added TBD (1.49 g, 10.6 mmol, CAS #5807-14-7). The mixture was stirred at 80° C. for 1 hr. On completion, the reaction mixture was concentrated in vacuo to give the crude product. The residue was purified by reversed-phase HPLC (column: Phenomenex luna C18 150*25 mm*10 um; mobile phase: [water(FA)-ACN]; B %: 30%-60%, 10 min) to give the title compound (300 mg, 56% yield) as a yellow solid. ¹H NMR (400 MHz, DMSO-d₆) δ=8.71 (s, 1H), 7.97 (d, J=9.6 Hz, 1H), 7.81 (s, 1H), 6.64 (d, J=9.2 Hz, 1H), 5.28-5.19 (m, 1H), 2.14-2.06 (m, 2H), 1.98-1.86 (m, 4H), 1.69-1.62 (m, 2H). LCMS (ESI⁺) m/z 249.2 (M+H)⁺.

### Synthesis of 7-((4-((7-Azaspiro[3.5]nonan-2-yl)sulfonyl)-2-methylphenyl)amino)-1-cyclopentyl-1,6-naphthyridin-2(1H)-one (Intermediate NI)

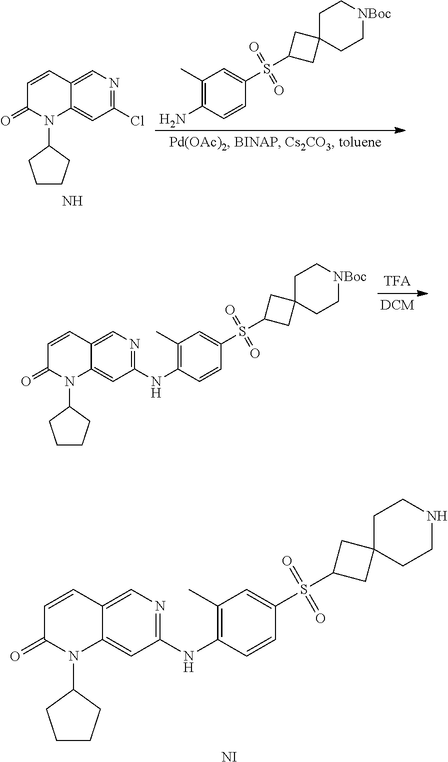

### Step 1—Tert-butyl 2-((4-((1-cyclopentyl-2-oxo-1,2-dihydro-1,6-naphthyridin-7-yl)amino)-3-methylphenyl)sulfonyl)-7-azaspiro[3.5]nonane-7-carboxylate

A mixture of 7-chloro-1-cyclopentyl-1,6-naphthyridin-2(1H)-one (295 mg, 1.19 mmol, Intermedia NH), tert-butyl 2-((4-amino-3-methylphenyl)sulfonyl)-7-azaspiro[3.5]nonane-7-carboxylate (1.03 g, 2.61 mmol, Intermediate PF), Pd(OAc)₂ (31.9 mg, 142 mol), BINAP (88.6 mg, 142 μmol) and Cs₂CO₃ (1.16 g, 3.56 mmol) in toluene (2 mL) was degassed and purged with N₂ three times. Then the mixture was stirred at 100° C. for 16 hrs under N₂ atmosphere. On completion, the reaction mixture was diluted with H₂O (80 mL) and extracted with EA (50 mL×3). The combined organic layers were washed with NaCl(aq) (50 mL), dried over Na₂SO₄, filtered and concentrated in vacuo to give the crude product. The residue was purified by reversed-phase HPLC (column: Phenomenex luna C18 150*25 mm*10 um; mobile phase: [water(FA)-ACN]; B %: 49%-79%, 10 min) to give the title compound (210 mg, 29% yield) as a white solid. ¹H NMR (400 MHz, DMSO-d₆) δ=8.73 (s, 1H), 8.51 (s, 1H), 8.07 (d, J=8.8 Hz, 1H), 7.83-7.78 (m, 1H), 7.68 (d, J=1.6 Hz, 1H), 7.61 (dd, J=2.0, 8.8 Hz, 1H), 7.05-7.00 (m, 1H), 6.37-6.32 (m, 1H), 5.29-5.20 (m, 1H), 4.11-4.02 (m, 1H), 3.25-3.18 (m, 4H), 2.39-2.36 (m, 3H), 2.15-2.05 (m, 4H), 2.00-1.87 (m, 6H), 1.68-1.61 (m, 2H), 1.51-1.47 (m, 2H), 1.45-1.41 (m, 2H), 1.37 (s, 9H). LCMS (ESI⁺) m/z 607.4 (M+H)⁺.

### Step 2-7-((4-((7-Azaspiro[3.5]nonan-2-yl)sulfonyl)-2-methylphenyl)amino)-1-cyclopentyl-1,6-naphthyridin-2(1H)-one

To a solution of tert-butyl 2-((4-((1-cyclopentyl-2-oxo-1,2-dihydro-1,6-naphthyridin-7-yl)amino)-3-methylphenyl)sulfonyl)-7-azaspiro[3.5]nonane-7-carboxylate (110 mg, 181 μmol) in DCM (1 mL) was added TFA (3 mL). The mixture was then stirred at 25° C. for 0.5 hr. On completion, the reaction mixture was concentrated in vacuo to give the title compound (90.0 mg, 97% yield) as a yellow oil liquid. LCMS (ESI⁺) m/z 507.3 (M+H)⁺.

### Synthesis of Tert-butyl 2-(4-amino-3-methyl-phenyl)sulfanyl-7-azaspiro[3.5]nonane-7-carboxylate (Intermediate NJ)

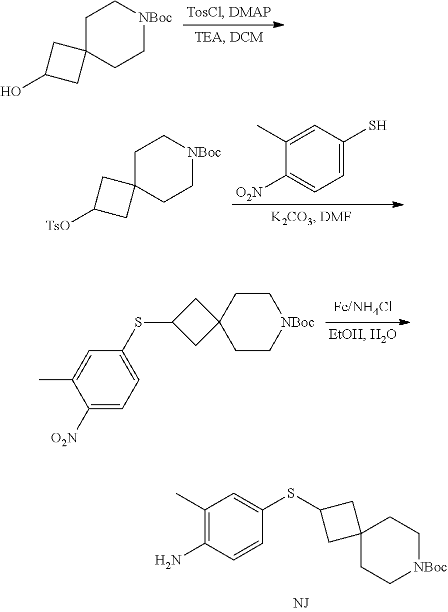 [SMILES: CC(C)(C)OC(=O)N1CCC2(CC1)CC(O)C2 ; Cc1ccc(S(=O)(=O)OC2CC3(CCN(C(=O)OC(C)(C)C)CC3)C2)cc1 ; Cc1cc(S)ccc1[N+](=O)[O-] ; Cc1cc(SC2CC3(CCN(C(=O)OC(C)(C)C)CC3)C2)ccc1[N+](=O)[O-] ; Cc1cc(SC2CC3(CCN(C(=O)OC(C)(C)C)CC3)C2)ccc1N]

### Step 1—Tert-butyl 2-(p-tolylsulfonyloxy)-7-azaspiro[3.5]nonane-7-carboxylate

To a solution of tert-butyl 2-hydroxy-7-azaspiro[3.5]nonane-7-carboxylate (10.0 g, 41.4 mmol, CAS #240401-28-9) in DCM (100 mL) was added TEA (12.5 g, 124 mmol), TosCl (11.8 g, 62.1 mmol) and DMAP (759 mg, 6.22 mmol). Then the mixture was stirred at 25° C. for 16 hrs. On completion, the mixture was concentrated in vacuo to give a residue and the residue was purified by column chromatography (SiO₂, Petroleum ether/Ethyl acetate=50/1 to 10/1) to give the title compound (17 g, 95% yield) as yellow solid. ¹H NMR (400 MHz, CDCl₃) δ 7.78 (d, J=8.4 Hz, 2H), 7.34 (d, J=8.0 Hz, 2H), 4.87-4.79 (m, 1H), 3.32-3.22 (m, 4H), 2.46 (s, 3H), 2.27-2.16 (m, 2H), 1.99-1.88 (m, 2H), 1.53-1.48 (m, 2H), 1.47-1.42 (m, 11H); LC-MS (ESI⁺) m/z 339.7 (M−56)⁺.

### Step 2—Tert-butyl 2-(3-methyl-4-nitro-phenyl)sulfanyl-7-azaspiro[3.5]nonane-7-carboxylate

To a solution of tert-butyl 2-(p-tolylsulfonyloxy)-7-azaspiro[3.5]nonane-7-carboxylate (14.0 g, 35.4 mmol) in DMF (150 mL) was added K₂CO₃ (14.6 g, 106 mmol) and 3-methyl-4-nitro-benzenethiol (11.9 g, 70.7 mmol). Then the mixture was stirred at 80° C. for 16 hrs. On completion, the mixture was filtered and the filtrate was concentrated in vacuo to give a residue. The residue was purified by column chromatography (SiO₂, Petroleum ether/Ethyl acetate=50/1 to 10/1) to give the title compound (13.0 g, 51% yield) as yellow solid. LC-MS (ESI⁺) m/z 337.3 (M−56)+.

### Step 3—Tert-butyl 2-(4-amino-3-methyl-phenyl)sulfanyl-7-azaspiro[3.5]nonane-7-carboxylate

To a solution of tert-butyl 2-(3-methyl-4-nitro-phenyl)sulfanyl-7-azaspiro[3.5]nonane-7-carboxylate (5.00 g, 12.7 mmol) and NH₄Cl (6.81 g, 127 mmol) in EtOH (50 mL) and H₂O (10 mL) was added Fe (3.56 g, 63.6 mmol). Then the mixture was stirred at 80° C. for 2 hrs. On completion, the mixture was filtered and the filter cake was washed with EtOH (3×100 mL). The filtrate was concentrated in vacuo to give the title compound (4.60 g, 62% yield) as yellow oil. ¹H NMR (400 MHz, DMSO-d₆) δ 7.00-6.95 (m, 1H), 6.94-6.89 (m, 1H), 6.56 (d, J=8.0 Hz, 1H), 5.00 (s, 2H), 3.64-3.51 (m, 1H), 3.23-3.11 (m, 4H), 2.18-2.09 (m, 2H), 2.03-1.98 (m, 3H), 1.71-1.61 (m, 2H), 1.47-1.44 (m, 2H), 1.38-1.36 (m, 9H), 1.34-1.24 (m, 2H); LC-MS (ESI⁺) m/z 307.4 (M−56)⁺.

### Synthesis of Tert-butyl 2-[4-[[4-chloro-5-(trifluoromethyl)pyrimidin-2-yl]amino]-3-methyl-phenyl]sulfonyl-7-azaspiro[3.5]nonane-7-carboxylate (Intermediate NK)

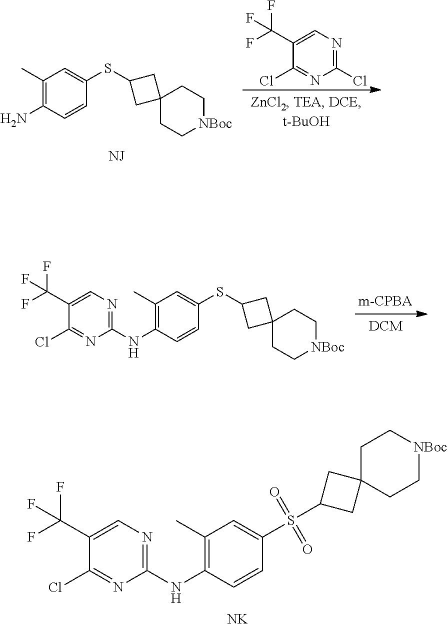 [SMILES: Cc1cc(SC2CC3(CCN(C(=O)OC(C)(C)C)CC3)C2)ccc1N ; FC(F)(F)c1cnc(Cl)nc1Cl ; Cc1cc(SC2CC3(CCN(C(=O)OC(C)(C)C)CC3)C2)ccc1Nc1ncc(C(F)(F)F)c(Cl)n1 ; Cc1cc(S(=O)(=O)C2CC3(CCN(C(=O)OC(C)(C)C)CC3)C2)ccc1Nc1ncc(C(F)(F)F)c(Cl)n1 ; "NK"]

### Step 1—Tert-butyl 2-[4-[[4-chloro-5-(trifluoromethyl)pyrimidin-2-yl]amino]-3-methyl-phenyl]sulfanyl-7-azaspiro[3.5]nonane-7-carboxylate

To a solution of 2,4-dichloro-5-(trifluoromethyl)pyrimidine (2.99 g, 13.7 mmol, CAS #3932-97-6) in t-BuOH (10 mL) and DCE (10 mL) was added dropwise ZnCl₂ (1.00 M, 16.5 mL) at 0° C. for 30 min. Then tert-butyl 2-(4-amino-3-methyl-phenyl)sulfanyl-7-azaspiro[3.5]nonane-7-carboxylate (5.00 g, 13.7 mmol, Intermediate NJ) in t-BuOH (10 mL) and DCE (10 mL) and TEA (15.1 mmol, 2.11 mL) were added dropwise at 0° C. Then the mixture was stirred at 25° C. for 14 hrs. On completion, the mixture was diluted with H₂O (50 mL), extracted with EA (3×20 mL) and washed with brine (2×20 mL). The organic layer was dried over anhydrous sodium sulfate, filtered and concentrated in vacuo to give the residue. The residue was purified by column chromatography (SiO₂, PE:EA=20:1 to 10:1) to give the title compound (6.00 g, 80% yield). ¹H NMR (400 MHz, DMSO-d₆) δ 1.38 (s, 9H), 1.44-1.48 (m, 2H), 1.54 (d, J=5.2 Hz, 2H), 1.71-1.80 (m, 2H), 2.17 (s, 3H), 2.35-2.43 (m, 2H), 3.18 (s, 2H), 3.26 (s, 2H), 3.90-4.03 (m, 1H), 7.07 (d, J=8.0 Hz, 1H), 7.13 (s, 1H), 7.27 (d, J=8.2 Hz, 1H), 8.65 (s, 1H), 10.05 (s, 1H). LC-MS (ESI⁺) m z 487.4 (M+H)⁺.

### Step 2—Tert-butyl 2-[4-[[4-chloro-5-(trifluoromethyl)pyrimidin-2-yl]amino]-3-methyl-phenyl]sulfonyl-7-azaspiro[3.5]nonane-7-carboxylate

To a solution of tert-butyl 2-[4-[[4-chloro-5-(trifluoromethyl)pyrimidin-2-yl]amino]-3-methyl-phenyl] sulfanyl-7-azaspiro[3.5]nonane-7-carboxylate (3.00 g, 5.52 mmol) in DCM (30 mL) was added m-CPBA (3.36 g, 16.6 mmol, 85% solution) at 0° C., then the mixture was stirred at 25° C. for 4 hrs. On completion, the mixture was quenched with saturated Na₂SO₃ (30 mL) and saturated Na₂CO₃ (30 mL) at 0° C., diluted with water (150 mL) and extracted with DCM (3×100 mL). The combined organic layer was anhydrous Na₂SO₄, filtered and concentrated in vacuo to give the residue. The residue was purified by column chromatography (SiO₂, PE:EA=10:1 to 5:1) to give the title compound (2.00 g, 63% yield) as a white solid. ¹H NMR (400 MHz, DMSO-d₆) δ 1.37 (s, 9H), 1.41-1.46 (m, 2H), 1.4-1.53 (m, 2H), 1.92-2.04 (m, 2H), 2.06-2.15 (m, 2H), 2.34 (s, 3H), 3.1-3.28 (m, 4H), 4.15 (m, 1H), 7.66-7.77 (m, 3H), 8.76 (s, 1H), 10.33 (s, 1H). LC-MS (ESI⁺) m/z 554.2 (M+H)⁺.

### Synthesis of Tert-butyl 2-[4-[[4-[(3S)-3-hydroxy-3-methyl-1-piperidyl]-5-(trifluoromethyl)pyrimidin-2-yl]amino]-3-methyl-phenyl]sulfonyl-7-azaspiro[3.5]nonane-7-carboxylate (Intermediate NL) and tert-butyl 2-[4-[[4-[(3R)-3-hydroxy-3-methyl-1-piperidyl]-5-(trifluoromethyl)pyrimidin-2-yl]amino]-3-methyl-phenyl]sulfonyl-7-azaspiro[3.5]nonane-7-carboxylate (Intermediate NM)

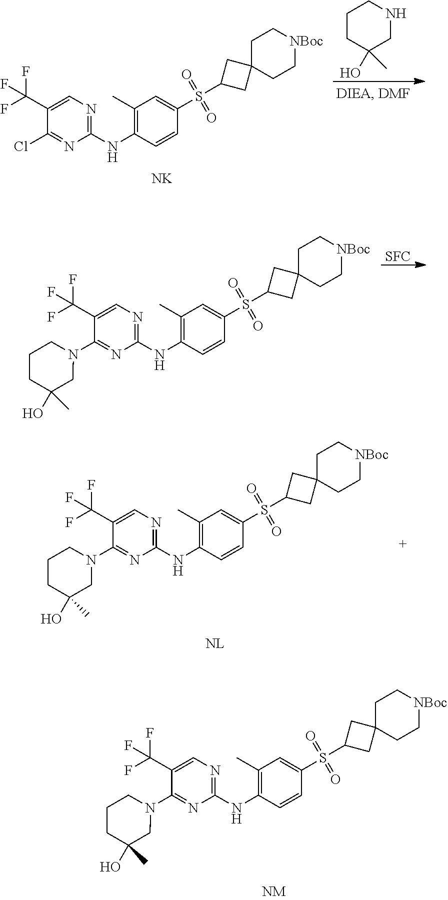

### Step 1—Tert-butyl 2-[4-[[4-[3-3-hydroxy-3-methyl-1-piperidyl]-5-(trifluoromethyl)pyrimidin-2-yl]amino]-3-methyl-phenyl]sulfonyl-7-azaspiro[3.5]nonane-7-carboxylate

To a solution of tert-butyl 2-[4-[[4-chloro-5-(trifluoromethyl)pyrimidin-2-yl]amino]-3-methyl-phenyl]sulfonyl-7-azaspiro[3.5]nonane-7-carboxylate (500 mg, 869 μmol, Intermediate NK) and 3-methylpiperidin-3-ol (120 mg, 1.04 mmol, CAS #473730-88-0) in DMF (5 mL) was added DIEA (1.74 mmol, 303 μL). Then the mixture was stirred at 25° C. for 2 hrs. On completion, the mixture was diluted with EA (20 mL) and water (60 mL), then extracted with EA (3×20 mL). The combined organic layer was dried over anhydrous Na₂SO₄, filtered and concentrated in vacuo to give the residue. The residue was purified by column chromography (SiO₂, PE:EA=1:1 to 1:5) to give the title compound (500 mg, 90% yield) as a white solid. ¹H NMR (400 MHz, DMSO-d₆) δ 1.04 (s, 3H), 1.13-1.26 (m, 1H), 1.37 (s, 9H), 1.40-1.43 (m, 2H), 1.47-1.50 (m, 2H), 1.54-1.58 (m, 2H), 1.78 (m, 1H), 1.93-1.99 (m, 3H), 2.06-2.11 (m, 2H), 2.35 (s, 3H), 3.17-3.23 (m, 4H), 3.26 (s, 1H), 3.30 (s, 1H) 3.39-3.43 (m, 1H), 3.56-3.68 (m, 1H) 4.03-4.12 (m, 1H), 4.46 (s, 1H), 7.62 (m, 1H), 7.67 (s, 1H), 7.97 (d, J=8.8 Hz, 1H), 8.36 (s, 1H), 9.11 (s, 1H). LC-MS (ESI⁺) m/z 654.3 (M+H)⁺.

### Step 2—Tert-butyl 2-[4-[[4-[(3S)-3-hydroxy-3-methyl-1-piperidyl]-5-(trifluoromethyl)pyrimidin-2-yl]amino]-3-methyl-phenyl]sulfonyl-7-azaspiro[3.5]nonane-7-carboxylate and tert-butyl 2-[4-[[4-[(3R)-3-hydroxy-3-methyl-1-piperidyl]-5-(trifluoromethyl)pyrimidin-2-yl]amino]-3-methyl-phenyl]sulfonyl-7-azaspiro[3.5]nonane-7-carboxylate

Tert-butyl 2-[4-[[4-[3-3-hydroxy-3-methyl-1-piperidyl]-5-(trifluoromethyl)pyrimidin-2-yl]amino]-3-methyl-phenyl]sulfonyl-7-azaspiro[3.5]nonane-7-carboxylate was separated by SFC (column: DAICEL CHIRALPAK IC (250 mm*30 mm, 10 um); mobile phase: [CO₂-ACN/MeOH (0.1% NH₃H₂O)]; B %:45%, isocratic elution mode) to give tert-butyl 2-[4-[[4-[(3S)-3-hydroxy-3-methyl-1-piperidyl]-5-(trifluoromethyl)pyrimidin-2-yl]amino]-3-methyl-phenyl]sulfonyl-7-azaspiro[3.5]nonane-7-carboxylate (100 mg, 18% yield, ¹H NMR (400 MHz, DMSO-d₆) δ 1.04 (s, 3H), 1.19-1.27 (m, 1H), 1.37 (s, 9H), 1.41 (d, J=5.4 Hz, 2H), 1.48 (d, J=4.8 Hz, 2H), 1.54-1.58 (m, 2H), 1.79 (m, 1H), 1.90-1.98 (m, 2H), 2.06-2.11 (m, 2H), 2.35 (s, 3H), 3.18-3.23 (m, 4H), 3.26 (s, 1H), 3.30 (s, 1H), 3.39-3.43 (m, 1H), 3.58-3.65 (m, 1H), 4.04-4.11 (m, 1H), 4.46 (s, 1H), 7.62 (m, 1H), 7.67 (s, 1H), 7.97 (d, J=8.8 Hz, 1H), 8.36 (s, 1H), 9.11 (s, 1H)) and tert-butyl 2-[4-[[4-[(3R)-3-hydroxy-3-methyl-1-piperidyl]-5-(trifluoromethyl)pyrimidin-2-yl]amino]-3-methyl-phenyl]sulfonyl-7-azaspiro[3.5]nonane-7-carboxylate (100 mg, 18% yield, ¹H NMR (400 MHz, DMSO-d₆) δ 1.04 (s, 3H), 1.37 (s, 9H), 1.40-1.43 (m, 2H), 1.47-1.50 (m, 2H), 1.54-1.59 (m, 2H), 1.78 (m, 1H), 1.92-2.02 (m, 2H), 2.04-2.13 (m, 2H), 2.35 (s, 3H), 3.16-3.24 (m, 4H), 3.26 (s, 1H), 3.30 (s, 1H), 3.37-3.46 (m, 2H), 3.56-3.68 (m, 1H), 4.05-4.15 (m, 1H), 4.46 (s, 1H), 7.58-7.73 (m, 2H), 7.97 (d, J=8.4 Hz, 1H), 8.36 (s, 1H), 9.11 (s, 1H)) as white solids. LC-MS (ESI⁺) m/z 654.4 (M+H)⁺ for both isomers. Absolute stereochemistry of the enantiomers was assigned arbitrarily.

### Synthesis of (3R)-1-[2-[4-(7-azaspiro[3.5]nonan-2-ylsulfonyl)-2-methyl-anilino]-5-(trifluoromethyl)pyrimidin-4-yl]-3-methyl-piperidin-3-ol (Intermediate NN)

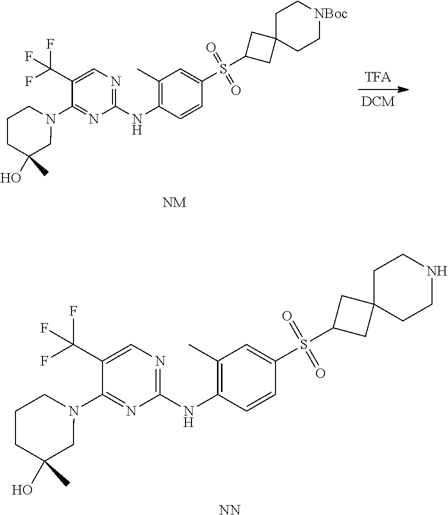 [SMILES: Cc1cc(S(=O)(=O)C2CC3(CCN(C(=O)OC(C)(C)C)CC3)C2)ccc1Nc1ncc(C(F)(F)F)c(N2CCC[C@@](C)(O)C2)n1 ; Cc1cc(S(=O)(=O)C2CC3(CCNCC3)C2)ccc1Nc1ncc(C(F)(F)F)c(N2CCC[C@@](C)(O)C2)n1 ; "NN"]

To a solution of tert-butyl 2-[4-[[4-[(3R)-3-hydroxy-3-methyl-1-piperidyl]-5-(trifluoromethyl)pyrimidin-2-yl]amino]-3-methyl-phenyl]sulfonyl-7-azaspiro[3.5]nonane-7-carboxylate (100 mg, 153 μmol, Intermediate NM) in DCM (5.00 mL) was added TFA (13.5 mmol, 1.00 mL). Then the mixture was stirred at 25° C. for 1 hr. On completion, the mixture was concentrated in vacuo to give the title compound (100 mg, 98% yield, TFA) as a yellow solid. LC-MS (ESI⁺) m/z 554.2 (M+H)⁺.

### (3S)-1-[2-[4-(7-azaspiro[3.5]nonan-2-ylsulfonyl)-2-methyl-anilino]-5-(trifluoromethyl)pyrimidin-4-yl]-3-methyl-piperidin-3-ol (Intermediate NO)

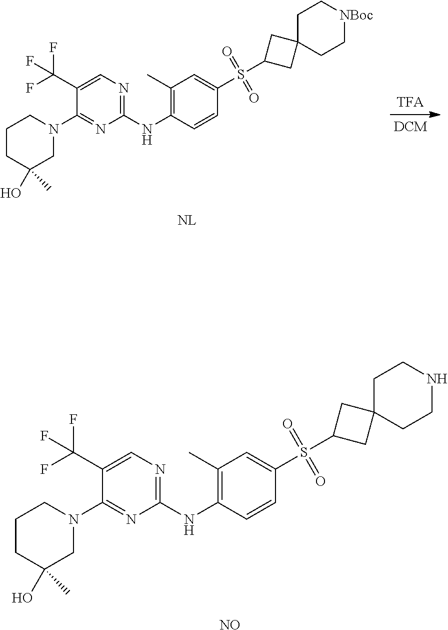 [SMILES: Cc1cc(S(=O)(=O)C2CC3(CCN(C(=O)OC(C)(C)C)CC3)C2)ccc1Nc1ncc(C(F)(F)F)c(N2CCC[C@](C)(O)C2)n1 ; Cc1cc(S(=O)(=O)C2CC3(CCNCC3)C2)ccc1Nc1ncc(C(F)(F)F)c(N2CCC[C@](C)(O)C2)n1 ; "NO"]

To a solution of tert-butyl2-[4-[[4-[(3S)-3-hydroxy-3-methyl-1-piperidyl]-5-(trifluoromethyl)pyrimidin-2-yl]amino]-3-methyl-phenyl]sulfonyl-7-azaspiro[3.5]nonane-7-carboxylate (100 mg, 153 μmol, Intermediate NL) in DCM (1 mL) was added TFA (153 μmol, 11.4 μL). Then the mixture was stirred at 25° C. for 0.5 hr. On completion, the mixture was concentrated in vacuo to give the title compound (101 mg, 98% yield) as a yellow solid. LC-MS (ESI⁺) m/z 554.2 (M+H)-.

Ethyl 1-(4-chloro-2-methylsulfonyl-pyrimidin-5-yl)cyclopropanecarboxylate (Intermediate NP)

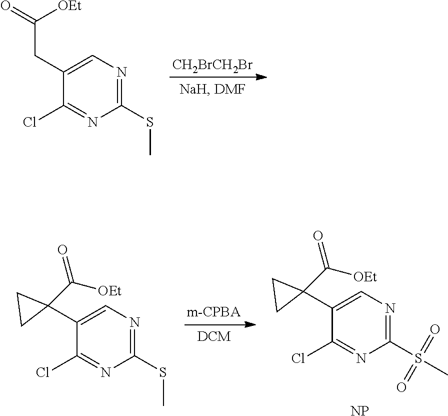

### Step 1—Ethyl 1-(4-chloro-2-methylsulfanyl-pyrimidin-5-yl)cyclopropanecarboxylate

To a solution of NaH (20.2 g, 506 mmol, 60% dispersion in mineral oil) in DMF (800 mL) added a solution of ethyl 2-(4-chloro-2-methylsulfanyl-pyrimidin-5-yl)acetate (50.0 g, 202 mmol, CAS #61727-34-2) and 1,2-dibromoethane (57.1 g, 304 mmol, 22.9 mL) in DMF (800 mL) at 0° C. The reaction mixture was stirred at 25° C. for 5 hr. On completion, the mixture was quenched with water (500 mL), then the mixture was extracted with EA (3×700 mL). The combined organic layer was dried over Na₂SO₄, filtered and filtrate was concentrated in vacuo. The residue was purified by column chromatography to give the title compound (34.0 g, 30% yield) as white solid. ¹H NMR (400 MHz, DMSO-d₆) δ 8.62 (s, 1H), 4.12-3.93 (m, 2H), 2.53 (s, 3H), 1.58 (d, J=3.2 Hz, 2H), 1.39-1.32 (m, 2H), 1.10 (t, J=7.2 Hz, 3H).

### Step 2—Ethyl 1-(4-chloro-2-methylsulfonyl-pyrimidin-5-yl)cyclopropanecarboxylate

To a solution of ethyl 1-(4-chloro-2-methylsulfanyl-pyrimidin-5-yl)cyclopropanecarboxylate (17.0 g, 62.3 mmol) in DCM (300 mL) was added m-CPBA (50.6 g, 249 mmol, 85% solution) at 0° C. The reaction mixture was stirred at 25° C. for 2 hrs. On completion, the reaction mixture was quenched by saturated Na₂S₂O₃ (50 mL) and saturated NaHCO₃ (50 mL) and diluted with water (100 mL), then the residue was extracted with DCM (3×200 mL). The combined organic layer was dried over Na₂SO₄, filtered and filtrate was concentrated in vacuo. The residue was purified by column chromatography to give the title compound (35.0 g, 92% yield) as white solid. ¹HNMR (400 MHz, DMSO-d₆) δ 9.10 (s, 1H), 4.08 (q, J=7.2 Hz, 2H), 3.46 (s, 3H), 1.69-1.64 (m, 2H), 1.53-1.47 (m, 2H), 1.11 (t, J=7.2 Hz, 3H); LC-MS (ESI⁺) m/z 305.0 (M+H)⁺.

### Synthesis of 2′-[4-(7-Azaspiro[3.5]nonan-2-ylsulfonyl)-2-methyl-anilino]-7′-[(1R,3R)-3-hydroxycyclohex yl]spiro[cyclopropane-1,5′-pyrrolo[2,3-d]pyrimidine]-6′-one (Intermediate NQ)

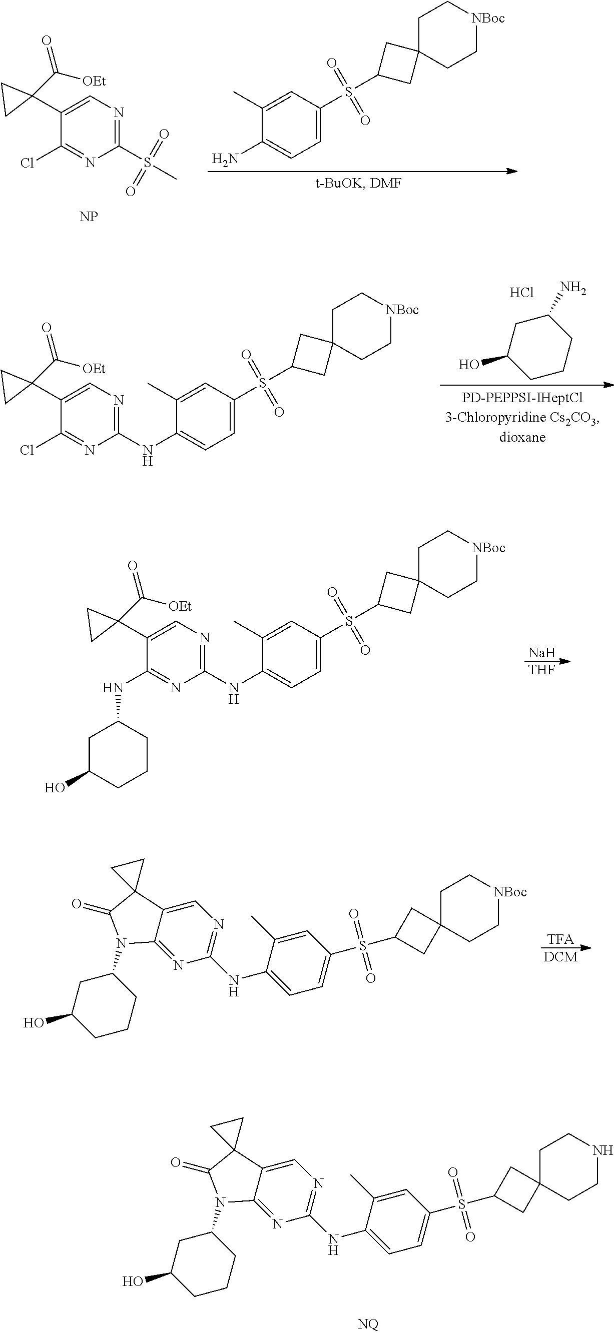

### Step 1—Tert-butyl 2-[4-[[4-chloro-5-(1-ethoxycarbonylcyclopropyl)pyrimidin-2-yl]amino]-3-methyl-phenyl]sulfonyl-7-azaspiro[3.5]nonane-7-carboxylate

To a solution of tert-butyl 2-(4-amino-3-methyl-phenyl)sulfonyl-7-azaspiro[3.5]nonane-7-carboxylate (6.47 g, 16.4 mmol, Intermediate PF) in DMF (50 mL) was added t-BuOK (3.68 g, 32.8 mmol) at 0° C., then ethyl 1-(4-chloro-2-methylsulfonyl-pyrimidin-5-yl)cyclopropanecarboxylate (5.00 g, 16.4 mmol, Intermediate NP) was added. The mixture then was stirred at 0° C. for 2 hrs. On completion, the residue was diluted with water (50 mL), then the residue was extracted with EA (3×60 mL). The combined organic layer was dried over Na₂SO₄, filtered and concentrated in vacuo to give a residue. The residue was purified by column chromatography and the residue was purified by reverse phase (0.1% FA condition) to give the title compound (2.90 g, 28% yield) as white solid. ¹H NMR (400 MHz, DMSO-d₆) δ 9.55 (s, 1H), 8.40 (s, 1H), 7.85 (d, J=8.4 Hz, 1H), 7.72-7.62 (m, 2H), 4.15-4.08 (m, 1H), 4.07-4.02 (m, 2H), 3.25-3.17 (m, 4H), 2.35 (s, 3H), 2.13-2.06 (m, 2H), 2.00-1.94 (m, 2H), 1.56 (d, J=3.2 Hz, 2H), 1.51-1.47 (m, 2H), 1.45-1.41 (m, 2H), 1.37 (s, 9H), 1.30 (d, J=2.8 Hz, 2H), 1.11 (t, J=7.2 Hz, 3H); LC-MS (ESI⁺) m/z 563.1 (M+H)⁺.

### Step 2—Tert-butyl 2-[4-[[5-(1-ethoxycarbonylcyclopropyl)-4-[[(1R,3R)-3-hydroxycyclohexyl]amino]pyrimidin-2-yl]amino]-3-methyl-phenyl]sulfonyl-7-azaspiro[3.5]nonane-7-carboxylate

To a mixture of tert-butyl 2-[4-[[4-chloro-5-(1-ethoxycarbonylcyclopropyl)pyrimidin-2-yl]amino]-3-methyl-phenyl]sulfonyl-7-azaspiro[3.5]nonane-7-carboxylate (1.00 g, 1.62 mmol) and (1R,3R)-3-aminocyclohexanol; hydrochloride (1.22 g, 6.46 mmol, HCl, CAS #1817645-57-0) in dioxane (10 mL) was added Cs₂CO₃ (2.10 g, 6.46 mmol) and 1,3-bis[2,6-bis(1-propylbutyl)phenyl]-4,5-dichloro-2H-imidazol-1-ium-2-ide; 3-chloropyridine; dichloropalladium (314 mg, 323 μmol). Then the mixture was stirred at 100° C. for 12 hrs. On completion, the residue was diluted with water (10 mL), then the mixture was extracted with EA (3×15 mL). The residue was purified by reverse phase (0.1% FA condition) to give the title compound (500 mg, 42% yield) as white solid. ¹H NMR (400 MHz, DMSO-d₆) δ 9.00 (s, 1H), 8.33 (d, J=8.4 Hz, 1H), 7.87 (s, 1H), 7.67-7.58 (m, 2H), 6.97 (s, 1H), 4.54-4.41 (m, 2H), 4.06-4.00 (m, 4H), 3.20 (s, 4H), 2.40 (s, 3H), 2.09-2.04 (m, 2H), 1.97-1.90 (m, 2H), 1.76-1.65 (m, 4H), 1.64-1.56 (m, 2H), 1.53 (s, 2H), 1.48 (s, 4H), 1.42 (s, 4H), 1.37 (s, 9H), 1.11 (t, J=7.2 Hz, 3H); LC-MS (ESI⁺) m/z 698.2 (M+H)⁺.

### Step 3—Tert-butyl 2-[4-[[7′-[(1R,3R)-3-hydroxycyclohexyl]-6′-oxo-spiro[cyclopropane-1,5′-pyrrolo [2,3-d]pyrimidine]-2′-yl]amino]-3-methyl-phenyl]sulfonyl-7-azaspiro[3.5]nonane-7-carboxylate

To a solution of tert-butyl 2-[4-[[5-(1-ethoxycarbonylcyclopropyl)-4-[[(1R,3R)-3-hydroxycyclohexyl]amino]pyrimidin-2-yl]amino]-3-methyl-phenyl]sulfonyl-7-azaspiro[3.5]nonane-7-carboxylate (500 mg, 716 mol) in THF (5 mL) was added NaH (57.3 mg, 1.43 mmol, 60% dispersion in mineral oil) at 0° C., then the mixture was stirred at 70° C. for 0.5 hr. On completion, the reaction mixture was quenched with water (0.5 mL) and the residue was diluted with water (5 mL), then the residue was extracted with EA (3×10 mL). The combined organic layer was dried over Na₂SO₄, filtered and concentrated in vacuo to give a residue. The residue was purified by column chromatography to give the title compound (170 mg, 36% yield) as white solid. ¹H NMR (400 MHz, DMSO-d₆) δ 8.92 (s, 1H), 7.99 (d, J=8.4 Hz, 1H), 7.94 (s, 1H), 7.66 (d, J=1.6 Hz, 1H), 7.60 (dd, J=2.0, 8.4 Hz, 1H), 4.71-4.62 (m, 1H), 4.56 (d, J=2.4 Hz, 1H), 4.12-4.06 (m, 2H), 3.24-3.17 (m, 4H), 2.42 (s, 1H), 2.36 (s, 3H), 2.16 (dd, J=2.8, 12.0 Hz, 1H), 2.12-2.06 (m, 2H), 1.97-1.90 (m, 2H), 1.72 (s, 1H), 1.69-1.66 (m, 2H), 1.63 (s, 2H), 1.53-1.46 (m, 6H), 1.43 (s, 2H), 1.37 (s, 9H), 1.30-1.24 (m, 1H); LC-MS (ESI⁺) m/z 652.3 (M+H)⁺.

### Step 4—2′-[4-(7-Azaspiro[3.5]nonan-2-ylsulfonyl)-2-methyl-anilino]-7′-[(1R,3R)-3-hydroxycyclohex yl]spiro[cyclopropane-1,5′-pyrrolo[2,3-d]pyrimidine]-6′-one

To a solution of tert-butyl 2-[4-[[7′-[(1R,3R)-3-hydroxycyclohexyl]-6′-oxo-spiro[cyclopropane-1,5′-pyrrolo[2,3-d]pyrimidine]-2′-yl]amino]-3-methyl-phenyl]sulfonyl-7-azaspiro[3.5]nonane-7-carboxylate (100 mg, 153 mol) in DCM (1 mL) was added TFA (17.4 mg, 153 μmol, 11.4 μL), then the mixture was stirred at 25° C. for 0.5 hr. On completion, the reaction mixture was concentrated in vacuo to give the title compound (100 mg, 97% yield, TFA) as yellow oil. LC-MS (ESI⁺) m/z 552.3 (M−100+H)⁺.

### Synthesis of 1-[4-[[Tert-butyl(diphenyl)silyl]oxymethyl]cyclohexoxy]propan-2-one (Intermediate NR)

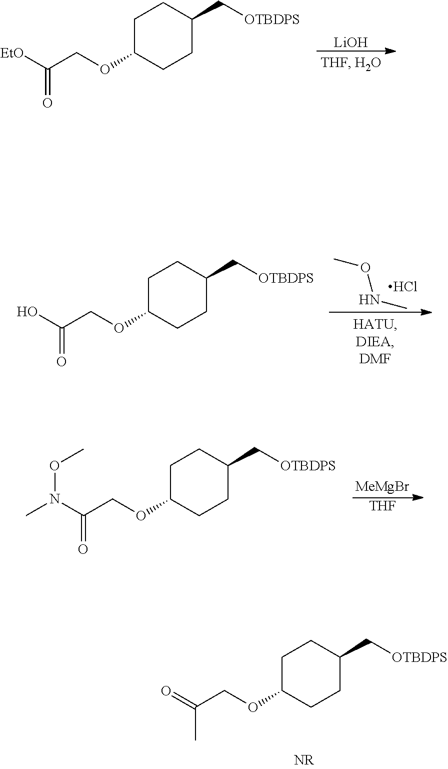

### Step 1—2-[4-[[Tert-butyl(diphenyl)silyl]oxymethyl]cyclohexoxy]acetic acid

To a solution of ethyl 2-[4-[[tertbutyl(diphenyl)silyl]oxymethyl]cyclohexoxy] acetate (5.20 g, 11.4 mmol, synthesized via Steps 1-3 of Intermediate NF) in a mixture of THF (25 mL) and H₂O (25 mL) was added LiOH·H₂O (719 mg, 17.1 mmol). The mixture was then stirred at 25° C. for 2 hrs. On completion, the reaction mixture was partitioned between H₂O (50 mL) and EA (30 mL×3). The organic phase was separated, washed with brine (20 mL×2), dried over anhydrous Na₂SO₄, filtered and concentrated in vacuo to give the title compound (4.80 g, 98% yield) as a yellow solid. LCMS (ESI⁺) m/z 449.2 (M+Na)⁺.

### Step 2—2-[4-[[Tert-butyl(diphenyl)silyl]oxymethyl]cyclohexoxy]-N-methoxy-N-methyl-acetamide

To a solution of 2-[4-[[tert-butyl(diphenyl)silyl]oxymethyl]cyclohexoxy]acetic acid (6.40 g, 15.0 mmol) and N-methoxymethanamine (1.46 g, 15.0 mmol, HCl, CAS #1117-97-1) in DMF (60 mL) was added DIEA (5.82 g, 45.0 mmol, 7.84 mL) and HATU (7.42 g, 19.5 mmol). The mixture was then stirred at 25° C. for 1 hr. On completion, the reaction mixture was partitioned between H₂O (150 mL) and EA (50 mL×3). The organic phase was separated, washed with brine (30 mL×2), dried over anhydrous Na₂SO₄, filtered and concentrated in vacuo to give a residue. The residue was purified by column chromatography (SiO₂, Petroleum ether/Ethyl acetate=10/1 to 5/1) to give the title compound (7.00 g, 99% yield) as a yellow oil. ¹H NMR (400 MHz, DMSO-d₆) δ 7.59 (dd, J=1.6, 7.6 Hz, 4H), 7.46-7.42 (m, 6H), 4.22 (s, 2H), 3.65 (s, 3H), 3.45 (d, J=6.0 Hz, 2H), 3.26-3.20 (m, 1H), 3.07 (s, 3H), 2.02 (d, J=9.6 Hz, 2H), 1.76 (d, J=11.6 Hz, 2H), 1.50-1.43 (m, 1H), 1.13 (d, J=12.8 Hz, 2H), 0.99 (s, 9H), 0.94 (d, J=2.0 Hz, 2H). LCMS (ESI⁺) m/z 470.6 (M+H)⁺.

### Step 3—1-[4-[[Tert-butyl(diphenyl)silyl]oxymethyl]cyclohexoxy]propan-2-one

To a solution of 2-[4-[[tert-butyl(diphenyl)silyl]oxymethyl]cyclohexoxy]-N-methoxy-N-methyl-acetamide (3.00 g, 6.39 mmol) in THF (30 mL) was degassed and purged with N₂ 3 times. Then MeMgBr (3 M, 6.39 mL) was added at 0° C., and the mixture was stirred at 25° C. for 2 hrs under N₂ atmosphere. On completion, the reaction mixture was quenched by addition of NH₄Cl (30 mL) at 0° C., and then extracted with EA (30 mL×3). The combined organic layers were washed with brine (20 mL×2), dried over anhydrous Na₂SO₄, filtered and concentrated in vacuo to give a residue. The residue was purified by column chromatography (SiO₂, Petroleum ether/Ethyl acetate=100/1 to 10/1) to give the title compound (2.40 g, 88% yield) as a white solid. ¹H NMR (400 MHz, DMSO-d₆) δ 7.62-7.57 (m, 4H), 7.47-7.40 (m, 6H), 4.09 (s, 2H), 3.45 (d, J=6.0 Hz, 2H), 3.22-3.13 (m, 1H), 2.03 (s, 3H), 2.02-1.96 (m, 2H), 1.80-1.72 (m, 2H), 1.52-1.42 (m, 1H), 1.19-1.09 (m, 2H), 0.99 (s, 9H), 0.98-0.90 (m, 2H). LCMS (ESI⁺) m/z 447.2 (M+Na)+.

### Synthesis of (2R)-1-[4-[[tert-butyl(diphenyl)silyl]oxymethyl]cyclohexoxy]-N-[(4-methoxyphenyl)methyl] propan-2-amine (Intermediate NS) and (2S)-1-[4-[[tert-butyl(diphenyl)silyl]oxymethyl]cyclohexoxy]-N-[(4-methoxyphenyl)methyl]propan-2-amine (Intermediate NT)

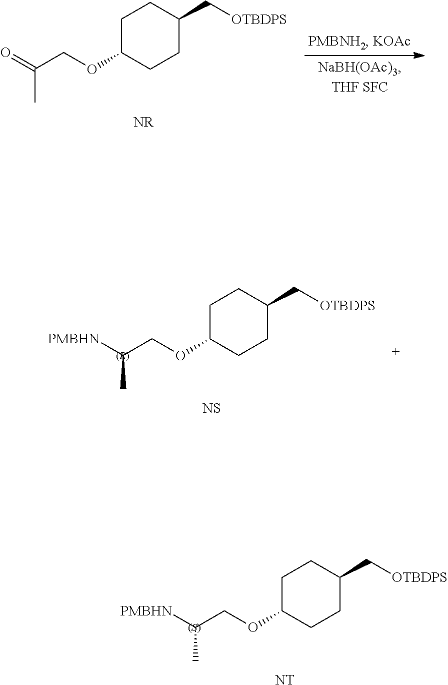

To a solution of 1-[4-[[tert-butyl(diphenyl)silyl]oxymethyl]cyclohexoxy]propan-2-one (2.5 g, 5.89 mmol, Intermediate NR) and PMBNH₂ (969 mg, 7.06 mmol) in THF (30 mL) was added HOAc (707 mg, 11.7 mmol) and NaBH(OAc)₃ (1.62 g, 7.65 mmol). The mixture was then stirred at 25° C. for 2 hrs. On completion, the mixture was quenched with H₂O (5 ml) and concentrated in vacuo. The residue was purified by column chromatography (SiO₂, DCM:MeOH=10:1) to give racemic compound. Then racemate was separated by SFC (column: DAICEL CHIRALPAK AS (250 mm*50 mm, 10 um); mobile phase: [CO₂-iPrOH (0.1% NH₃H₂O)]; B %:40%, isocratic elution mode) to give (2R)-1-[4-[[tert-butyl(diphenyl)silyl]oxymethyl]cyclohexoxy]-N-[(4-methoxyphenyl)methyl] propan-2-amine (1 g, 31% yield, peak 2, HNMR (EC9037-244-PK2): ¹H NMR (400 MHz, CDCl₃) δ 7.58 (d, J=6.4 Hz, 4H), 7.38-7.27 (m, 6H), 7.21-7.18 (m, 2H), 6.79 (d, J=8.4 Hz, 2H), 3.78 (d, J=12.8 Hz, 1H), 3.72 (s, 3H), 3.63 (d, J=12.8 Hz, 1H), 3.41-3.35 (m, 3H), 3.29 (d, J=8.0 Hz, 1H), 3.07 (t, J=4.0 Hz, 1H), 2.91-2.80 (m, 1H), 1.95 (d, J=5.2 Hz, 2H), 1.75 (d, J=11.2 Hz, 2H), 1.46-1.37 (m, 1H), 1.24-1.17 (m, 1H), 1.13-1.05 (m, 2H), 1.01 (d, J=6.4 Hz, 3H), 0.97 (s, 9H), 0.95-0.81 (m, 2H)) as yellow oil and (2S)-1-[4-[[tert-butyl(diphenyl)silyl]oxymethyl]cyclohexoxy]-N-[(4-methoxyphenyl)methyl] propan-2-amine (1 g, 31% yield, peak 1, HNMR (EC9037-244-PK1): ¹H NMR (400 MHz, CDCl₃) δ 7.68-7.62 (m, 4H), 7.45-7.35 (m, 6H), 7.30 (d, J=8.4 Hz, 2H), 6.88 (d, J=8.4 Hz, 2H), 3.96 (d, J=12.8 Hz, 1H), 3.84-3.79 (m, 3H), 3.79-3.74 (m, 1H), 3.50 (d, J=4.0 Hz, 1H), 3.45 (d, J=6.0 Hz, 2H), 3.42 (s, 1H), 3.21-3.12 (m, 1H), 3.01 (d, J=4.8 Hz, 1H), 2.04-1.98 (m, 2H), 1.88-1.78 (m, 2H), 1.51-1.48 (m, 1H), 1.22 (d, J=6.0 Hz, 2H), 1.18 (d, J=3.0 Hz, 1H), 1.15 (d, J=6.4 Hz, 3H), 1.05 (s, 9H), 0.98 (d, J=14.4 Hz, 2H)) as yellow oil. The absolute stereochemistry of the diastereomers was confirmed by reference comparison.

Tert-butyl N-[(1R)-2-(4-formylcyclohexoxy)-1-methyl (Intermediate NU)

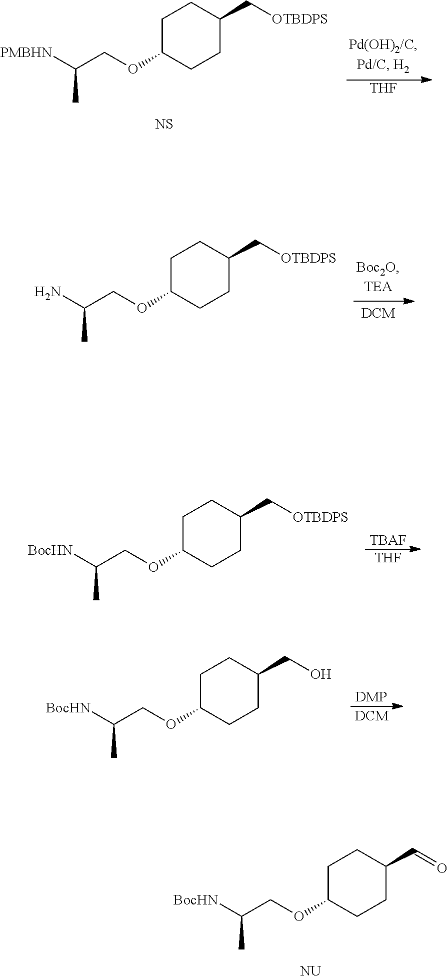

### Step 1—(2R)-1-[4-[[tert-butyl(diphenyl)silyl]oxymethyl]cyclohexoxy]propan-2-amine

To a solution of (2R)-1-[4-[[tert-butyl(diphenyl)silyl]oxymethyl]cyclohexoxy]-N-[(4-methoxyphenyl) methyl]propan-2-amine (1 g, 1.83 mmol, Intermediate NS) in THF (20 mL) was added Pd/C (500 mg, 469 mol, 10 wt %) and Pd(OH)₂/C (500 mg, 1.83 mmol, 10 wt %) under N₂. The suspension was degassed under vacuum and purged with H₂ several times. The mixture was then stirred under H₂ (50 psi) at 50° C. for 48 hrs. On completion, the mixture was concentrated in vacuo to give the title compound (500 mg, 64% yield) as brown oil. LC-MS (ESI⁺) m/z 426.2 (M+H)⁺.

### Step 2—Tert-butyl N-[(1R)-2-[4-[[tert-butyl(diphenyl)silyl]oxymethyl]cyclohexoxy]-1-methyl-ethyl]carbamate

To a solution of (2R)-1-[4-[[tert-butyl(diphenyl)silyl]oxymethyl]cyclohexoxy]propan-2-amine (500 mg, 1.17 mmol) and TEA (237 mg, 2.35 mmol) in DCM (10 mL) was added Boc₂O (384 mg, 1.76 mmol). The reaction mixture was then stirred at 25° C. for 1.5 hrs. On completion, the mixture was diluted with DCM (10 mL) and washed with water (10 mL). The organic layer was dried over Na₂SO₄, filtered and the filtrate was concentrated in vacuo. The residue was purified by column chromatography (SiO₂, Petroleum ether/Ethyl acetate=I/O to 20/1) to give the title compound (290 mg, 46% yield) as colorless oil. H NMR (400 MHz, DMSO-d₆) δ 7.65-7.56 (m, 4H), 7.46-7.37 (m, 6H), 6.63-6.55 (m, 1H), 3.65-3.59 (m, 1H), 3.55-3.50 (m, 1H), 3.45 (d, J=6.0 Hz, 2H), 3.30 (s, 1H), 3.20-3.13 (m, 2H), 1.96 (d, J=13.6 Hz, 2H), 1.78-1.71 (m, 2H), 1.52-1.49 (m, 2H), 1.37 (s, 9H), 1.24-1.20 (m, 2H), 1.00-0.97 (m, 12H).

### Step 3—Tert-butyl N-[(1R)-2-[4-(hydroxymethyl)cyclohexoxy]-1-methyl-ethyl]carbamate

To a solution of tert-butyl N-[(1R)-2-[4-[[tert-butyl(diphenyl)silyl]oxymethyl]cyclohexoxy]-1-methyl-ethyl]carbamate (290 mg, 551 mol) in THF (4 mL) was added TBAF (1 M, 827 μL). The reaction mixture was stirred at 25° C. for 7 hrs. On completion, the mixture was concentrated in vacuo. The residue was purified by column chromatography (SiO₂, Petroleum ether/Ethyl acetate=10/1 to 1/1) to give the title compound (90 mg, 56% yield) as colorless oil. ¹H NMR (400 MHz, DMSO-d₆) δ 7.65-7.56 (m, 4H), 7.46-7.37 (m, 6H), 6.63-6.55 (m, 1H), 3.65-3.59 (m, 1H), 3.55-3.50 (m, 1H), 3.45 (d, J=6.0 Hz, 2H), 3.30 (s, 1H), 3.20-3.13 (m, 2H), 1.96 (d, J=13.6 Hz, 2H), 1.78-1.71 (m, 2H), 1.52-1.49 (m, 2H), 1.37 (s, 9H), 1.24-1.20 (m, 2H), 1.00-0.97 (m, 12H).

### Step 4—Tert-butyl N-[(1R)-2-(4-formylcyclohexoxy)-1-methyl-ethyl]carbamate

To a solution of tert-butyl N-[(1R)-2-[4-(hydroxymethyl)cyclohexoxy]-1-methyl-ethyl]carbamate (90.0 mg, 313 mol) in DCM (2 mL) was added DMP (199 mg, 469 μmol). The reaction mixture was then stirred at 25° C. for 3 hrs. On completion, the mixture was quenched by Na₂S₂O₃ (2 mL) and NaHCO₃ (2 mL). Then, the mixture was diluted with DCM (20 mL), washed with NaHCO₃ aqueous (15 mL) and water (15 mL×2). The organic layer was dried over Na₂SO₄, filtered and the filtrate was concentrated in vacuo to give the title compound (67 mg, 74% yield) as colorless oil. LC-MS (ESI⁺) m/z 286.3 (M+H)⁺.

### Synthesis of 3-(4-fluoro-3-methyl-2-oxo-5-piperazin-1-yl-benzimidazol-1-yl)piperidine-2,6-dione (Intermediate NV)

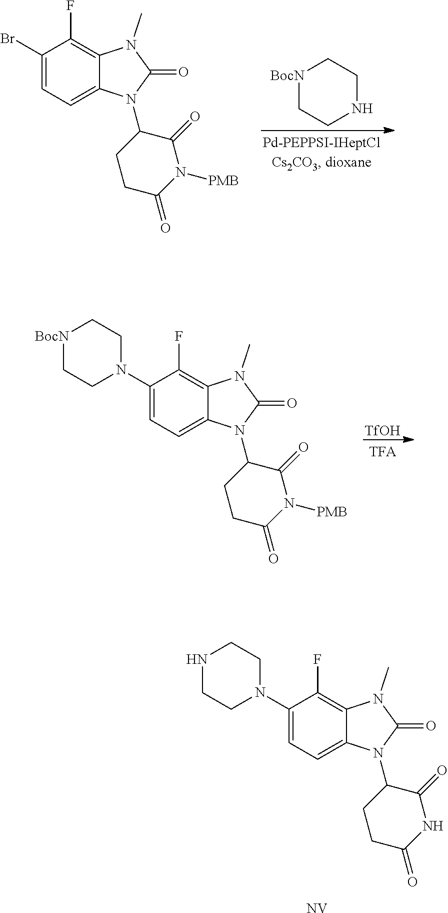

### Step 1—Tert-butyl 4-[4-fluoro-1-[1-[(4-methoxyphenyl)methyl]-2,6-dioxo-3-piperidyl]-3-methyl-2-oxo-benzimidazol-5-yl]piperazine-1-carboxylate

To a solution of 3-(5-bromo-4-fluoro-3-methyl-2-oxo-benzimidazol-1-yl)-1-[(4-methoxyphenyl)methyl]piperidine-2,6-dione (1 g, 2.10 mmol, synthesized via Steps 1-4 of Intermediate MU) and tert-butyl piperazine-1-carboxylate (469 mg, 2.52 mmol, CAS #143238-38-4) in dioxane (15 mL) was added 1,3-bis[2,6-bis(1-propylbutyl) phenyl]-4,5-dichloro -2H-imidazole -1-ium -2-ide; 3-chloropyridine; dichloropalladium (204 mg, 209 umol) and Cs₂CO₃ (1.37 g, 4.20 mmol). The mixture was then stirred at 100° C. under N₂ for 3 hrs. On completion, the mixture was filtered and diluted with H₂O (40 mL) and extracted with EA (40 mL×3). The combined organic layers were dried over Na₂SO₄, filtered and concentrated under reduced pressure. The residue was purified by column chromatography (SiO₂, Petroleum ether/Ethyl acetate=10/1 to 2/1) to give the title compound (1.0 g, 82% yield) as brown solid. LC-MS (ESI⁺) m/z 582.6 (M+H)⁺.

### Step 2—3-(4-fluoro-3-methyl-2-oxo-5-piperazin-1-yl-benzimidazol-1-yl)piperidine-2,6-dione

To a solution of tert-butyl 4-[4-fluoro-1-[1-[(4-methoxyphenyl) methyl]-2,6-dioxo -3-piperidyl]-3-methyl-2-oxo-benzimidazol-5-yl] piperazine-1-carboxylate (0.35 g, 601 umol) in TFA (4 mL) was added TfOH (1 mL). The mixture was then stirred at 70° C. for 1 hr. On completion, the mixture was concentrated in vacuo to give the title compound (200 mg, 92% yield) as yellow oil. LC-MS (ESI⁺) m z 362.1 (M+H)⁺.

### Synthesis of 3-[5-[4-[[4-[(2R)-2-aminopropoxy]cyclohexyl]methyl]piperazin-1-yl]-4-fluoro-3-methyl-2-oxo-benzimidazol-1-yl]piperidine-2,6-dione (Intermediate NW)

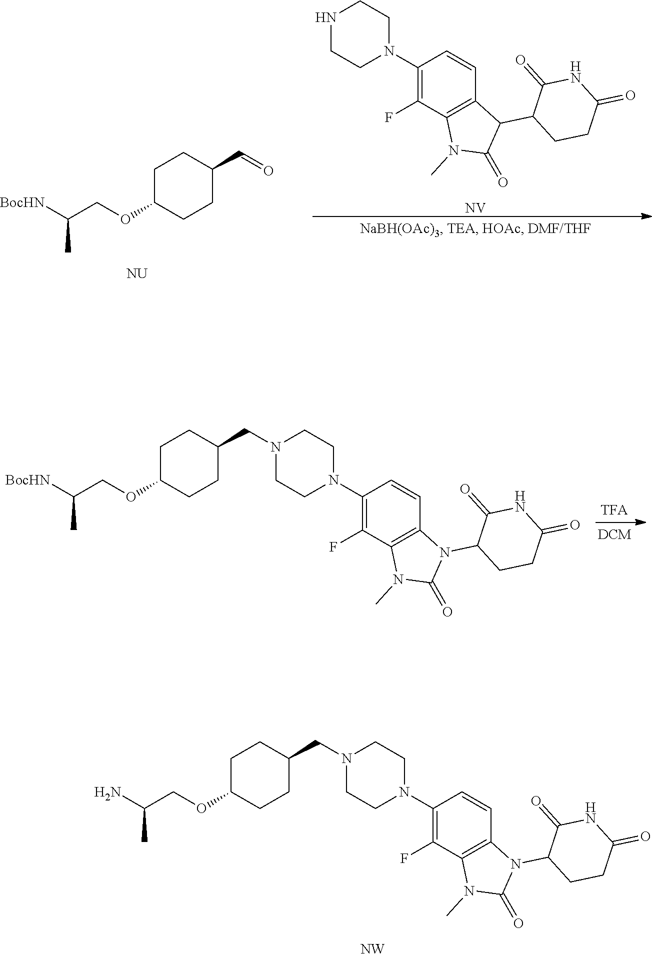

### Step 1—Tert-butyl N-[(1R)-2-[4-[[4-[1-(2,6-dioxo-3-piperidyl)-4-fluoro-3-methyl-2-oxo-benzimidazol-5-yl]piperazin-1-yl]methyl]cyclohexoxy]-1-methyl-ethyl]carbamate

To a solution of 3-(4-fluoro-3-methyl-2-oxo-5-piperazin-1-yl-benzimidazol-1-yl)piperidine-2,6-dione (160 mg, 336 μmol, TFA, Intermediate NV) in THF (1 mL) was added TEA (23.7 mg, 234 μmol) until the pH=8 and the mixture was stirred for 0.1 hr, Then, to the above mixture was added HOAc (28.20 mg, 469.55 μmol) and tert-butyl N-[(1R)-2-(4-formylcyclohexoxy)-1-methyl-ethyl]carbamate (67.00 mg, 234 mol, Intermediate NU) at 0° C. and then mixture was stirred for 0.5 hr. Next, NaBH(OAc)₃ (99.5 mg, 469 mol) was added at 0° C., and the mixture was stirred at 0° C. for 0.5 hr. On completion, the mixture was quenched by H₂O (0.05 mL) and concentrated in vacuo. The residue was purified by prep-HPLC (column: Welch ultimate C18 150*25 mm*7 um; mobile phase: [water (FA)-ACN]; gradient: 11%-41% B over 10 min) to give the title compound (35 mg, 23% yield) as white solid. ¹H NMR (400 MHz, CDCl₃) δ 8.12 (d, J=1.6 Hz, 1H), 6.71 (t, J=8.0 Hz, 1H), 6.51 (d, J=8.4 Hz, 1H), 5.17 (dd, J=5.2, 12.8 Hz, 1H), 4.69 (dd, J=2.0, 3.6 Hz, 1H), 3.78 (dd, J=3.2, 6.4 Hz, 1H), 3.61 (d, J=1.6 Hz, 3H), 3.46-3.37 (m, 2H), 3.24 (s, 4H), 3.00-2.85 (m, 5H), 2.83-2.66 (m, 2H), 2.51 (s, 2H), 2.32-2.19 (m, 2H), 2.07 (d, J=9.6 Hz, 2H), 1.97-1.94 (m, 2H), 1.70-1.63 (m, 1H), 1.45 (s, 9H), 1.23 (d, J=12.0 Hz, 2H), 1.16 (d, J=6.4 Hz, 3H), 1.09-1.01 (m, 2H).

### Step 2—3-[5-[4-[[4-[(2R)-2-aminopropoxy]cyclohexyl]methyl]piperazin-1-yl]-4-fluoro-3-methyl-2-oxo-benzimidazol-1-yl]piperidine-2,6-dione

A mixture of tert-butyl N-[(1R)-2-[4-[[4-[1-(2,6-dioxo-3-piperidyl)-4-fluoro-3-methyl-2-oxo-benzimidazol-5-yl]piperazin-1-yl]methyl]cyclohexoxy]-1-methyl-ethyl]carbamate (35 mg, 55.4 μmol) in TFA (0.2 mL) and DCM (1 mL) was stirred at 25° C. for 0.5 hr. On completion, the mixture was concentrated in vacuo to give the title compound (35 mg, 97% yield, TFA) as brown oil. LC-MS (ESI⁺) m/z 531.3 (M+H)⁺.

### Synthesis of 4-[[4-(3-Hydroxy-3-methyl-1-piperidyl)-5-(trifluoromethyl)pyrimidin-2-yl]amino]-3-methyl-benzenesulfonyl chloride (Intermediate NX)

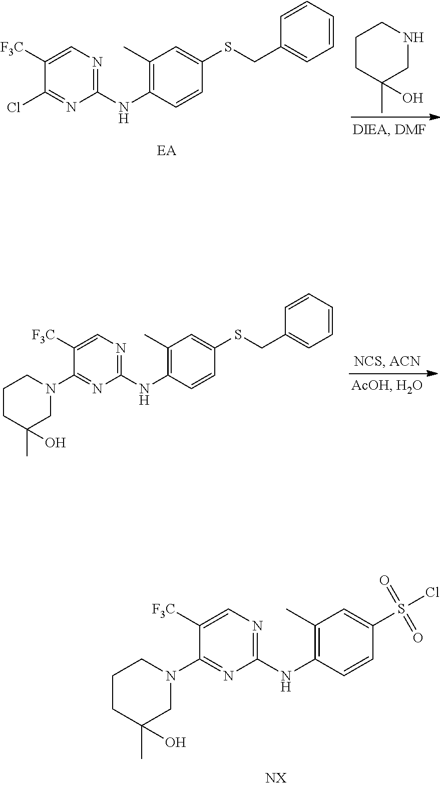

### Step 1—1-[2-(4-benzylsulfanyl-2-methyl-anilino)-5-(trifluoromethyl)pyrimidin-4-yl]-3-methyl -piperidin-3-ol

To a solution of 3-methylpiperidin-3-ol (168 mg, 1.46 mmol, CAS #473730-88-0) and DIEA (473 mg, 3.66 mmol, 637 μL) in DMF (7 mL) was added N-(4-benzylsulfanyl-2-methyl-phenyl)-4-chloro-5-(trifluoromethyl) pyrimidin-2-amine (500 mg, 1.22 mmol, Intermediate EA). The reaction was stirred at 25° C. for 2 hrs. On completion, the reaction was diluted with EA (40 mL). The organic layer was washed with water (40 mL×2), dried over Na₂SO₄ and concentrated in vacuo. The residue was purified by column chromatography (SiO₂, Petroleum ether/Ethyl acetate=5/1 to 3/1) to give the title compound (590 mg, 98% yield) as red solid. ¹H NMR (400 MHz, CDCl₃) δ 8.31 (s, 1H), 7.66 (d, J=8.4 Hz, 1H), 7.31-7.29 (m, 3H), 7.26-7.18 (m, 3H), 7.01-6.85 (m, 1H), 4.10 (s, 1H), 4.02 (d, J=13.6 Hz, 1H), 3.88 (d, J=12.8 Hz, 1H), 3.18-3.08 (m, 1H), 2.97 (d, J=13.6 Hz, 1H), 2.88-2.74 (m, 1H), 2.25 (s, 3H), 1.93-1.84 (m, 1H), 1.77 (d, J=13.6 Hz, 2H), 1.64-1.45 (m, 3H), 1.17 (s, 3H); LC-MS (ESI⁺) m/z 489.5 (M+H)⁺.

### Step 2—4-[[4-(3-hydroxy-3-methyl-1-piperidyl)-5-(trifluoromethyl)pyrimidin-2-yl]amino]-3-methyl -benzenesulfonyl chloride

To a solution of 1-[2-(4-benzylsulfanyl-2-methyl-anilino)-5-(trifluoromethyl)pyrimidin-4-yl]-3-methyl-piperidin-3-ol (50 mg, 102 μmol) in ACN (3 mL) and HOAc (0.3 mL) was added NCS (32.8 mg, 245 μmol) and H₂O (18.4 μg, 1.02 mol) in the dark. The reaction was stirred at 25° C. for 0.5 hr. On completion, the reaction was diluted with EA (50 mL). The organic layer was washed with water (50 mL×2), dried over with Na₂SO₄ and concentrated in vacuo to give the title compound (45 mg, 94% yield) as red oil. LC-MS (ESI⁺) m/z 465.1 (M+H)⁺.

### Synthesis of 3-(3-Methyl-2-oxo-4-piperazin-1-yl-benzimidazol-1-yl)piperidine-2,6-dione (Intermediate NY)

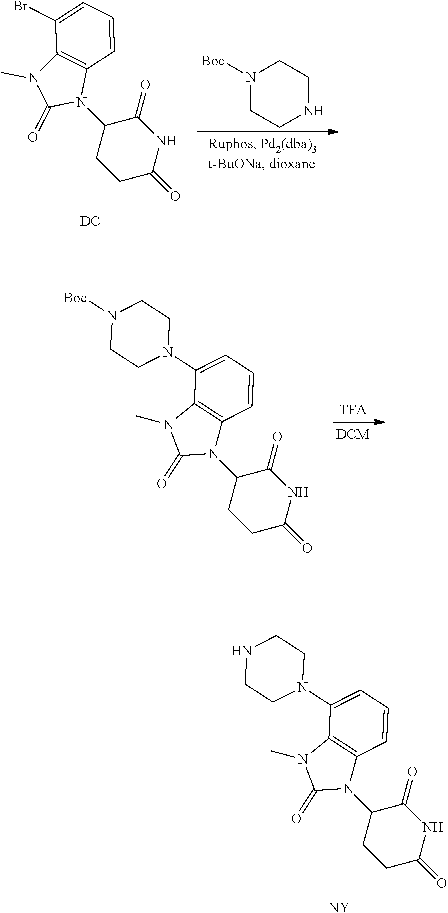 [SMILES: Cn1c(=O)n(C2CCC(=O)NC2=O)c2cccc(Br)c21 ; CC(C)(C)OC(=O)N1CCNCC1 ; "DC" ; Cn1c(=O)n(C2CCC(=O)NC2=O)c2cccc(N3CCN(C(=O)OC(C)(C)C)CC3)c21 ; Cn1c(=O)n(C2CCC(=O)NC2=O)c2cccc(N3CCNCC3)c21 ; "NY"]

### Step 1—Tert-butyl 4-[1-(2,6-dioxo-3-piperidyl)-3-methyl-2-oxo-benzimidazol-4-yl] piperazine-1-carboxylate

A solution of 3-(4-bromo-3-methyl-2-oxo-benzimidazol-1-yl)piperidine-2,6-dione (4.50 g, 13.3 mmol, Intermediate DC), tert-butyl piperazine-1-carboxylate (3.22 g, 17.3 mmol, CAS #143238-38-4), t-BuONa (3.84 g, 39.9 mmol), RuPhos (620 mg, 1.33 mmol) and Pd₂(dba)₃ (243 mg, 266 μmol) in dioxane (45 mL) was stirred at 100° C. for 24 hrs under N₂. On completion, the mixture was concentrated in vacuo to give the residue. The residue was purified by column chromatography (SiO₂, PE:EA=10:1 to 1:4) to give the target product (500 mg, 9% yield) as yellow solid. ¹H NMR (400 MHz, DMSO-d₆) δ 11.09 (s, 1H), 7.01-6.95 (m, 1H), 6.94-6.89 (m, 2H), 5.75 (s, 1H), 5.36 (dd, J=5.2, 12.4 Hz, 1H), 3.95 (d, J=2.4 Hz, 2H), 3.63 (s, 3H), 3.10-3.02 (m, 4H), 2.94-2.82 (m, 2H), 2.69 (dd, J=4.4, 12.8 Hz, 2H), 2.64-2.59 (m, 1H), 2.05-1.91 (m, 1H), 1.43 (s, 9H), 1.22-1.15 (m, 1H). LC-MS (ESI⁺) m/z 444.1 (M+H)⁺.

### Step 2—3-(3-Methyl-2-oxo-4-piperazin-1-yl-benzimidazol-1-yl)piperidine-2,6-dione

To a solution of tert-butyl 4-[1-(2,6-dioxo-3-piperidyl)-3-methyl-2-oxo-benzimidazol-4-yl]piperazine-1-carboxylate (120 mg, 270 μmol) in DCM (6 mL) was added TFA (1.17 g, 10.3 mmol), then the mixture was stirred at 25° C. for 0.5 hr. On completion, the mixture was concentrated in vacuo to give the title compound (110 mg, 88% yield) as purple solid. LC-MS (ESI⁺) m/z 344.0 (M+H)⁺.

### Synthesis of 3-[4-[4-[[4-[(2R)-2-aminopropoxy]cyclohexyl]methyl]piperazin-1-yl]-3-methyl-2-oxo-benzimidazol-1-yl]piperidine-2,6-dione (Intermediate NZ)

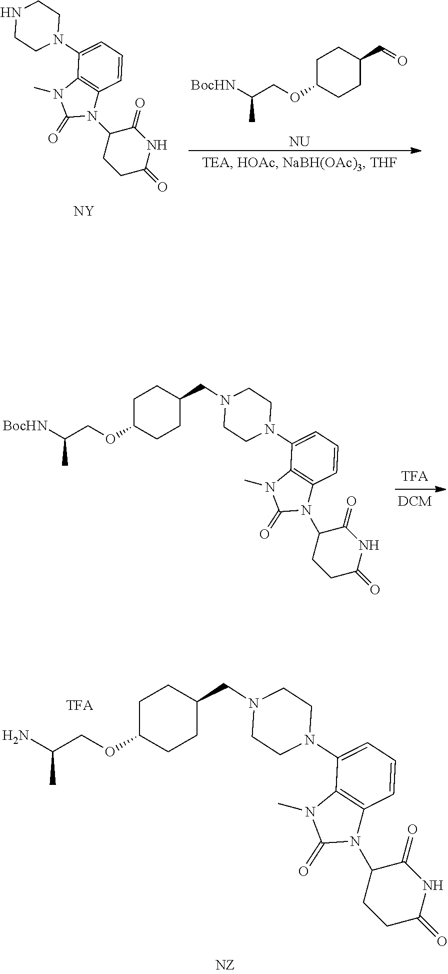

### Step 1—Tert-butyl N-[(1R)-2-[4-[[4-[1-(2,6-dioxo-3-piperidyl)-3-methyl-2-oxo-benzimidazol-4-yl]piperazin-1-yl]methyl]cyclohexoxy]-1-methyl-ethyl]carbamate

To a solution of 3-(3-methyl-2-oxo-4-piperazin-1-yl-benzimidazol-1-yl)piperidine-2,6-dione (110 mg, 240 mol, Intermediate NY) in THF (3 mL) was added TEA (24.3 mg, 240 mol) until pH=8-10. Then tert-butyl N-[(1R)-2-(4-formylcyclohexoxy)-1-methyl-ethyl]carbamate (130 mg, 455 μmol, Intermediate NU) in DMF (0.2 mL) and HOAc (14.4 mg, 240 mol) was added until the pH=4-5, and the mixture was stirred at −10° C. for 0.5 hr. Next, NaBH(OAc)₃ (101 mg, 480 μmol) was added, then the mixture was stirred at −10° C. for 0.5 hr. On completion, the mixture was concentrated in vacuo to give the title compound (100 mg, 67% yield) as white solid. ¹H NMR (400 MHz, DMSO-d₆) δ 11.09 (s, 1H), 8.15 (s, 1H), 7.00-6.95 (m, 1H), 6.94-6.91 (m, 1H), 6.88 (d, J=7.2 Hz, 1H), 6.60 (d, J=8.0 Hz, 1H), 5.35 (dd, J=5.2, 12.8 Hz, 1H), 3.61 (s, 3H), 3.56-3.49 (m, 1H), 3.34 (s, 3H), 3.17 (dd, J=6.8, 9.2 Hz, 2H), 2.94-2.81 (m, 6H), 2.69-2.63 (m, 3H), 2.14 (d, J=7.2 Hz, 2H), 1.99-1.92 (m, 3H), 1.79 (d, J=12.0 Hz, 2H), 1.46 (d, J=4.0 Hz, 1H). LC-MS (ESI⁺) m/z 613.3 (M+H)⁺.

### Step 2—3-[4-[4-[[4-[(2R)-2-aminopropoxy]cyclohexyl]methyl]piperazin-1-yl]-3-methyl-2-oxo-benzimidazol-1-yl]piperidine-2,6-dione

To a solution of tert-butyl N-[(1R)-2-[4-[[4-[1-(2,6-dioxo-3-piperidyl)-3-methyl-2-oxo-benzimidazol-4-yl]piperazin-1-yl]methyl]cyclohexoxy]-1-methyl-ethyl]carbamate (60.0 mg, 97.9 μmol) in DCM (1 mL) was added TFA (614 mg, 5.39 mmol), then the mixture was stirred at 25° C. for 0.5 hr. On completion, the mixture was concentrated in vacuo to give the title compound (55 mg, 79% yield) as brown oil. LC-MS (ESI⁺) m/z 513.3 (M+H)⁺.

### Synthesis of 4-[[4-[(3S)-3-hydroxy-3-methyl-1-piperidyl]-5-(trifluoromethyl)pyrimidin-2-yl]amino]-3-methyl-benzenesulfonyl chloride (Intermediate OA)

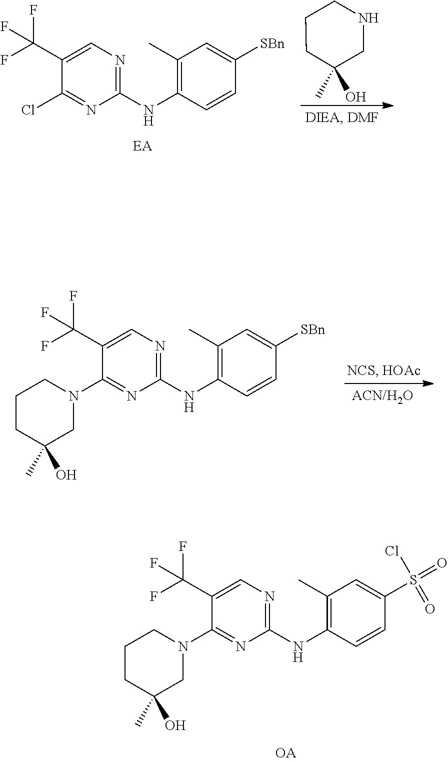

### Step 1—(3S)-1-[2-(4-benzylsulfanyl-2-methyl-anilino)-5-(trifluoromethyl)pyrimidin-4-yl]-3-methyl-piperidin-3-ol

To a mixture of N-(4-benzylsulfanyl-2-methyl-phenyl)-4-chloro-5-(trifluoromethyl)pyrimidin-2-amine (500 mg, 1.22 mmol, Intermediate EA) and (3S)-3-methylpiperidin-3-ol (140 mg, 1.22 mmol, CAS#1200132-32-6) in DMF (5 mL) was added DIEA (236 mg, 1.83 mmol). The reaction mixture was then stirred at 25° C. for 3 hrs. On completion, the residue was diluted with water (10 mL), then the residue was extracted with EA (30 mL×3). The combined organic layers were dried over Na₂SO₄, filtered and filtrate was concentrated in vacuo. The residue was purified by reverse phase (0.1% FA condition) to give the title compound (100 mg, 16% yield) as white solid. ¹H NMR (400 MHz, DMSO-d₆) δ 8.87 (s, 1H), 8.26 (s, 1H), 7.45-7.37 (m, 1H), 7.35-7.21 (m, 6H), 7.13 (d, J=8.8 Hz, 1H), 5.75 (s, 1H), 4.43 (s, 1H), 4.19 (s, 2H), 3.56-3.46 (m, 1H), 3.39-3.30 (m, 2H), 2.17 (s, 3H), 1.78-1.68 (m, 1H), 1.61-1.50 (m, 2H), 1.42 (d, J=2.4 Hz, 1H), 1.02 (s, 3H); LC-MS (ESI⁺) m/z 489.6 (M+H).

### Step 2—4-[[4-[(3S)-3-hydroxy-3-methyl-1-piperidyl]-5-(trifluoromethyl)pyrimidin-2-yl]amino]-3-methyl-benzenesulfonyl chloride

To a solution of (3S)-1-[2-(4-benzylsulfanyl-2-methyl-anilino)-5-(trifluoromethyl) pyrimidin-4-yl]-3-methyl-piperidin-3-ol (90.0 mg, 184 μmol) in ACN (1 mL), HOAc (0.1 mL) and H₂O (0.01 mL) was added NCS (73.7 mg, 552 μmol) in the dark. The reaction mixture was then stirred at 25° C. for 0.5 hr. On completion, the reaction was quenched with water (5 mL), then the mixture was extracted with EA (10 mL×3). The combined organic layers were dried over Na₂SO₄, filtered and filtrate was concentrated in vacuo to give the title compound (60.0 mg, 70% yield) as white oil. LC-MS (ESI⁺) m/z 465.2 (M+H).

### Synthesis of Tert-butyl N-[(1S)-2-[4-[[tert-butyl(diphenyl)silyl]oxymethyl]cyclohexoxy]-1-deuterio-1-methyl-ethyl]carbamate (Intermediate OB) and tert-butyl N-[(1R)-2-[4-[[tert-butyl(diphenyl)silyl]oxymethyl] cyclohexoxy]-1-deuterio-1-methyl-ethyl]carbamate (Intermediate OC)

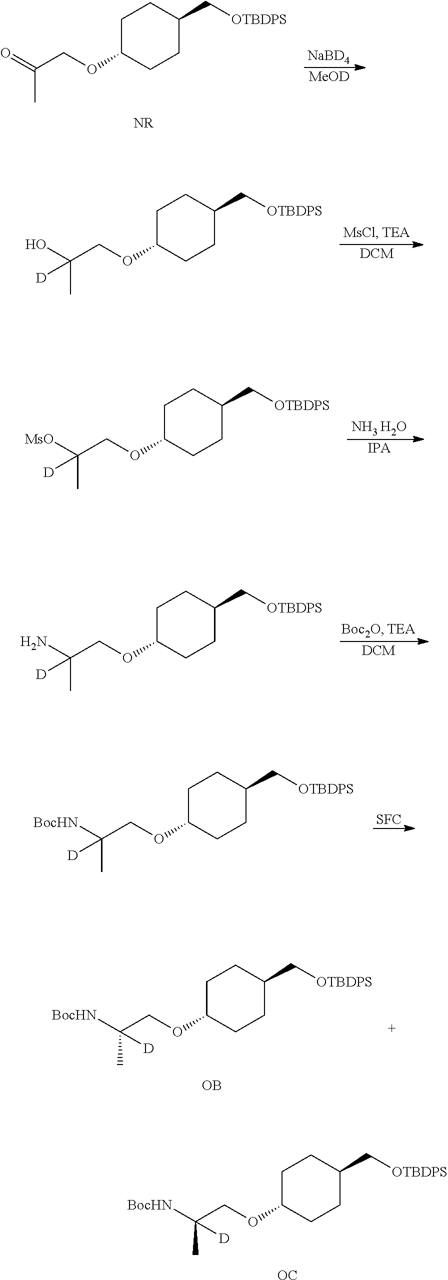

### Step 1—1-[4-[[Tert-butyl(diphenyl)silyl]oxymethyl]cyclohexoxy]-2-deuterio-propan-2-ol

To a solution of 1-[4-[[tert-butyl(diphenyl)silyl]oxymethyl]cyclohexoxy]propan-2-one (3 g, 7.06 mmol, Intermediate NR) in trideuterio(deuteriooxy)methane (20 mL) was added sodium tetradeuterioboranuide (294 mg, 7.77 mmol) at 0° C. Then the reaction was stirred at 25° C. for 1 hr. On completion, the reaction was quenched with NH₄Cl/D₂O (3 mL) and concentrated in vacuo. The residue was dissolved with EA (70 mL). The organic layer was washed with water (50 mL×2), dried over Na₂SO₄ and concentrated in vacuo to give the title compound (2.7 g, 89% yield) as colorless oil. ¹HNMR (400 MHz, CDCl₃) δ 7.69-7.63 (m, 4H), 7.46-7.36 (m, 6H), 3.53-3.45 (m, 3H), 3.25-3.17 (m, 2H), 2.09-2.03 (m, 2H), 1.89-1.82 (m, 2H), 1.56-1.47 (m, 1H), 1.27-1.19 (m, 2H), 1.14 (s, 3H), 1.06 (s, 9H), 1.04-0.94 (m, 2H).

### Step 2—[2-[4-[[tert-butyl(diphenyl)silyl]oxymethyl]cyclohexoxy]-1-deuterio-1-methyl-ethyl]methanesulfonate

To a solution of 1-[4-[[tert-butyl(diphenyl)silyl]oxymethyl]cyclohexoxy]-2-deuterio-propan-2-ol (2.6 g, 6.08 mmol) and TEA (1.85 g, 18.2 mmol, 2.54 mL) in DCM (30 mL) was added MsCl (1.31 g, 11.4 mmol, 885 μL) at 0° C. The reaction was then stirred at 25° C. for 1 hr. On completion, the reaction was quenched with water (10 mL) and diluted with DCM (70 mL). The organic layer was washed with water (50 mL×2), dried over Na₂SO₄ and concentrated in vacuo to give the title compound (2.9 g, 94% yield). ¹HNMR (400 MHz, CDCl₃) 7.58 (dd, J=1.6, 8.0 Hz, 4H), 7.38-7.28 (m, 6H), 3.50-3.43 (m, 2H), 3.39 (d, J=6.0 Hz, 2H), 3.17-3.10 (m, 1H), 2.99 (s, 3H), 2.01-1.93 (m, 2H), 1.80-1.72 (m, 2H), 1.47-1.38 (m, 1H), 1.31 (s, 3H), 1.19-1.09 (m, 2H), 0.97 (s, 9H), 0.96-0.86 (m, 2H), LC-MS (ESI⁺) m/z 506.3 (M+H)⁺.

### Step 3—1-[4-[[tert-butyl(diphenyl)silyl]oxymethyl]cyclohexoxy]-2-deuterio-propan-2-amine

A solution of [2-[4-[[tert-butyl(diphenyl)silyl]oxymethyl]cyclohexoxy]-1-deuterio-1-methyl-ethyl] methanesulfonate (1.3 g, 2.57 mmol) in NH₃H₂O (18.2 g, 124 mmol, 20 mL, 24% solution) and IPA (30 mL) was stirred under 50 psi at 70° C. for 48 hrs in a 100 mL of sealed tube. On completion, the reaction was concentrated in vacuo. Then the residue was dissolved with DCM (80 mL) and washed with water (50 mL×2). The organic layer was dried over Na₂SO₄ and concentrated in vacuo to give the title compound (2 g, 91% yield) as yellow oil. LC-MS (ESI⁺) m/z 427.3 (M+H)⁺.

### Step 4—Tert-butyl N-[2-[4-[[tert-butyl(diphenyl)silyl]oxymethyl]cyclohexoxy]-1-deuterio-1-methyl-ethyl]carbamate

To a solution of 1-[4-[[tert-butyl(diphenyl)silyl]oxymethyl]cyclohexoxy]-2-deuterio-propan-2-amine (1.8 g, 4.22 mmol) in DCM (20 mL) was added TEA (853 mg, 8.44 mmol, 1.17 mL) and Boc₂O (1.01 g, 4.64 mmol, 1.07 mL). The mixture was then stirred at 20° C. for 1 hr. On completion, the reaction was concentrated in vacuo. The crude product was purified by column chromatography (SiO₂, PE:EA=50:1 to 3:1, PE:EA=5:1, Rf=0.4) to give the title compound (2.8 g) yellow oil. LC-MS (ESI⁺) m/z 427.3 (M−100+H)⁺.

### Step 5—Tert-butyl N-[(1S)-2-[4-[[tert-butyl(diphenyl)silyl]oxymethyl]cyclohexoxy]-1-deuterio-1-methyl-ethyl]carbamate and tert-butyl N-[(1R)-2-[4-[[tert-butyl(diphenyl)silyl]oxymethyl] cyclohexoxy]-1-deuterio-1-methyl-ethyl]carbamate

Tert-butyl N-[2-[4-[[tert-butyl(diphenyl)silyl]oxymethyl]cyclohexoxy]-1-deuterio-1-methyl-ethyl]carbamate (2.8 g) was separated by SFC (column: DAICEL CHIRALPAK AD (250 mm*30 mm, 10 um); mobile phase: [CO2-i-PrOH (0.1% NH₃H₂O)]; B %:15%, isocratic elution mode) to give tert-butyl N-[(1S)-2-[4-[[tert-butyl(diphenyl)silyl]oxymethyl]cyclohexoxy]-1-deuterio-1-methyl-ethyl] carbamate (600 mg, 22% yield) as yellow oil (¹H NMR (400 MHz, DMSO-d₆) δ 7.60 (d, J=6.4 Hz, 4H), 7.50-7.34 (m, 6H), 6.59 (s, 1H), 3.45 (d, J=5.6 Hz, 2H), 3.30 (s, 1H), 3.20-3.08 (m, 2H), 1.96 (d, J=9.6 Hz, 2H), 1.76 (d, J=11.6 Hz, 2H), 1.50-1.43 (m, 1H), 1.38 (s, 9H), 1.19-1.02 (m, 4H), 1.01-0.95 (m, 12H), LC-MS (ESI⁺) m/z 427.1 (M−100+H)+) and tert-butyl-N-[(1R)-2-[4-[[tert-butyl(diphenyl)silyl]oxymethyl]cyclohexoxy]-1-deuterio-1-methyl-ethyl]carbamate (570 mg, 21% yield) as yellow oil (¹H NMR (400 MHz, DMSO-d₆) δ 7.59 (s, 4H), 7.45 (s, 6H), 6.81-6.51 (m, 1H), 3.50-3.41 (m, 2H), 3.31-3.26 (m, 1H), 3.21-3.07 (m, 2H), 1.96 (d, J=10.8 Hz, 2H), 1.83-1.69 (m, 2H), 1.52-1.45 (m, 1H), 1.38 (s, 9H), 1.09 (d, J=11.6 Hz, 4H), 1.00 (s, 12H); LC-MS (ESI⁺) m/z 427.1 (M−100+H)+). The absolute stereochemistry was assigned by comparison with a reference.

### Synthesis of Tert-butyl N-[(1R)-1-deuterio-2-(4-formylcyclohexoxy)-1-methyl-ethyl]carbamate (Intermediate OD)

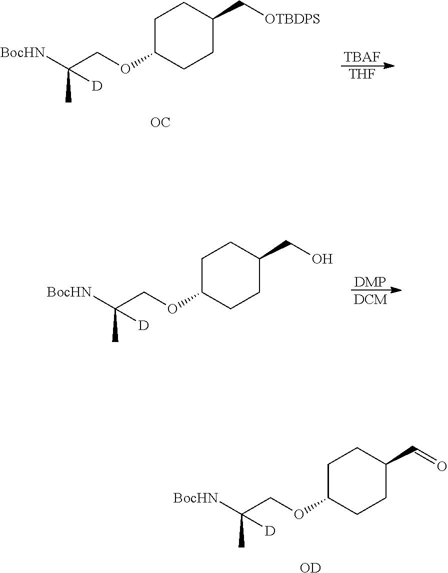

### Step 1—3-[4-[4-[[2-[4-[(6-Chloro-8-cyclopentyl-7-oxo-pyrido[2,3-d]pyrimidin-2-yl)amino]-3-methyl-phenyl]sulfonyl-7-azaspiro[3.5]nonan-7-yl]methyl]-1-piperidyl]-3-ethoxy-anilino]piperidine-2,6-dione

To a solution of tert-butyl N-[(1R)-2-[4-[[tert-butyl(diphenyl)silyl]oxymethyl]cyclohexoxy]-1-deuterio-1-methyl-ethyl]carbamate (700 mg, 1.33 mmol, Intermediate OC) in THF (3 mL) was added TBAF (1 M, 1.99 mL), the mixture was then stirred at 25° C. for 1 hr. On completion, the reaction mixture was diluted with EA (10 mL) and washed with water (10 mL×2), the organic layer was dried over Na₂SO₄, filtered and concentrated in vacuo. The residue was purified by column chromatography (SiO₂, PE:EA=10:1 to PE:EA=1:1) to give the title compound (380 mg, 99% yield) as yellow oil. ¹H NMR (400 MHz, DMSO-d₆) δ 6.59 (s, 1H), 3.30 (s, 2H), 3.21-3.17 (m, 2H), 3.15 (s, 1H), 3.14-3.06 (m, 1H), 1.95 (d, J=10.0 Hz, 2H), 1.72 (d, J=11.2 Hz, 2H), 1.38 (s, 9H), 1.32-1.24 (m, 1H), 1.13-1.01 (m, 2H), 0.98 (s, 3H), 0.94-0.81 (m, 2H).

### Step 2—Tert-butyl N-[(1R)-1-deuterio-2-(4-formylcyclohexoxy)-1-methyl-ethyl]carbamate

To a solution of tert-butyl N-[(1R)-1-deuterio-2-[4-(hydroxymethyl)cyclohexoxy]-1-methyl-ethyl]carbamate (370 mg, 1.28 mmol) in DCM (6 mL) was added DMP (707 mg, 1.67 mmol, 516 μL) at 0° C., then the reaction was stirred at 25° C. for 0.5 hr. On completion, the reaction mixture was quenched with saturated Na₂S₂O₃ solution (10 mL) and saturated NaHCO₃ (10 mL) under stirring for 10 min. The mixture was then extracted with DCM (2×10 mL). The combined organic layers were dried over Na₂SO₄, filtered and concentrated in vacuo to give the title compound (350 mg, 95% yield) as yellow oil. LC-MS (ESI⁺) m z 287.2 (M+H)⁺.

### Synthesis of Tert-butyl N-[(1R)-1-deuterio-2-(4-formylcyclohexoxy)-1-methyl-ethyl]carbamate (Intermediate OE)

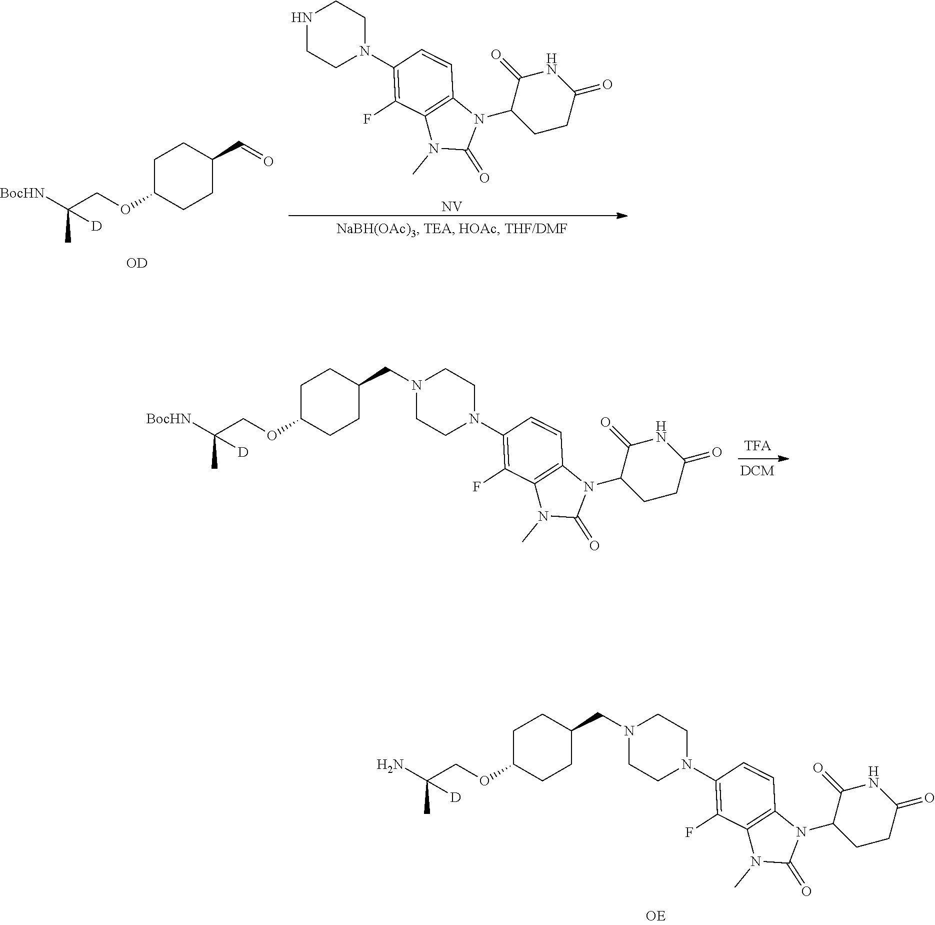

### Step 1—Tert-butyl N-[(1R)-1-deuterio-2-[4-[[4-[1-(2,6-dioxo-3-piperidyl)-4-fluoro-3-methyl-2-oxo-benzimidazol-5-yl]piperazin-1-yl]methyl]cyclohexoxy]-1-methyl-ethyl]carbamate

To a solution of 3-(4-fluoro-3-methyl-2-oxo-5-piperazin-1-yl-benzimidazol-1-yl)piperidine-2,6-dione (580 mg, 1.22 mmol, TFA, Intermediate NV) in THF (2 mL) was added TEA (618 mg, 6.11 mmol, 850 μL), HOAc (220 mg, 3.67 mmol, 209 μL) and tert-butyl N-[(1R)-1-deuterio-2-(4-formylcyclohexoxy)-1-methyl-ethyl]carbamate (350 mg, 1.22 mmol, Intermediate OD) at 0° C. Then the mixture was stirred at 25° C. for 0.3 hr, and NaBH(OAc)₃ (388 mg, 1.83 mmol) was added and the reaction was stirred at 25° C. for 0.5 hr. On completion, the reaction was quenched with water (5 mL) and concentrated in vacuo. The reaction was purified by prep-HPLC (column: Phenomenex luna C18 150*25 mm*10 um; mobile phase: [water (FA)-ACN]; gradient: 15%-45% B over 10 min) to give the title compound (220 mg, 28% yield) as white solid. ¹H NMR (400 MHz, DMSO-d₆) δ 11.12 (s, 1H), 6.96-6.84 (m, 1H), 6.80-6.69 (m, 1H), 6.61 (s, 1H), 5.40-5.29 (m, 1H), 3.48 (s, 3H), 3.32-3.29 (m, 2H), 3.17 (d, J=9.6 Hz, 2H), 3.02-2.84 (m, 4H), 2.81-2.54 (m, 4H), 2.46-2.36 (m, 2H), 2.13 (s, 1H), 2.09-1.87 (m, 4H), 1.84-1.76 (m, 2H), 1.53-1.41 (m, 1H), 1.38 (s, 9H), 1.20-1.06 (m, 2H), 0.99 (s, 3H), 0.96-0.74 (m, 2H).

### Step 2—Tert-butyl N-[(1R)-1-deuterio-2-(4-formylcyclohexoxy)-1-methyl-ethyl]carbamate

A solution of tert-butyl N-[(1R)-1-deuterio-2-[4-[[4-[1-(2,6-dioxo-3-piperidyl)-4-fluoro-3-methyl-2-oxo-benzimidazol-5-yl]piperazin-1-yl]methyl]cyclohexoxy]-1-methyl-ethyl]carbamate (70 mg, 110 mol) in DCM (0.9 mL) and TFA (0.3 mL) was stirred at 25° C. for 0.5 hr. On completion, the mixture was concentrated in vacuo to give the title compound (70 mg, 97% yield, TFA) as yellow oil. LC-MS (ESI⁺) m/z 532.1 (M+H)⁺.

### Synthesis of Benzyl 4-((1s,3s)-3-((tert-butoxycarbonyl)amino)-3-methylcyclobutyl)piperazine-1-carboxylate (Intermediate OF) and benzyl 4-((1r,3r)-3-((tert-butoxycarbonyl)amino)-3-methylcyclobutyl)piperazine-1-carboxylate (Intermediate OG)

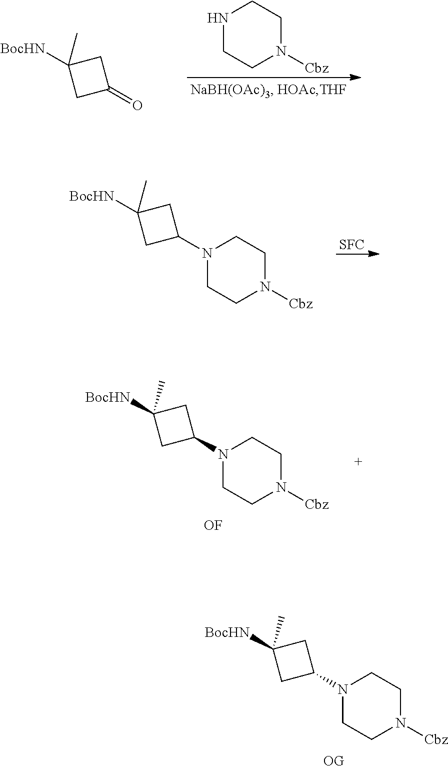

### Step 1—Benzyl 4-[3-(tert-butoxycarbonylamino)-3-methyl-cyclobutyl]piperazine-1-carboxylate

To a solution of benzyl piperazine-1-carboxylate (2.21 g, 10.0 mmol, 1.94 mL, CAS #31166-44-6) and tert-butyl N-(1-methyl-3-oxo-cyclobutyl)carbamate (2.00 g, 10.0 mmol, CAS #1523617-99-3) in THF (20 mL) was added HOAc (602 mg, 10.0 mmol, 574 μL) until the pH=4, then the mixture was stirred at 25° C. for 0.1 hr. Finally NaBH(OAc)₃ (3.19 g, 15.0 mmol) was added and the mixture was stirred at 25° C. for 0.5 hr. On completion, the reaction mixture was filtered and concentrated in vacuo to give a residue. The residue was purified by reversed-phase (0.1% FA condition) to give the title compound (1.10 g, 27% yield) as white solid. H NMR (400 MHz, DMSO-d₆) δ 7.37 (s, 5H), 7.13-6.98 (m, 1H), 5.09 (s, 2H), 2.44 (s, 4H), 2.15 (s, 5H), 1.39-1.37 (m, 12H), 1.32-1.28 (m, 4H). LC-MS (ESI⁺) m/z 404.1 (M+H)⁺.

### Step 2—Benzyl 4-((1s,3s)-3-((tert-butoxycarbonyl)amino)-3-methylcyclobutyl)piperazine-1-carboxylate and benzyl 4-((1r,3r)-3-((tert-butoxycarbonyl)amino)-3-methylcyclobutyl)piperazine-1-carboxylate

Benzyl 4-[3-(tert-butoxycarbonylamino)-3-methyl-cyclobutyl]piperazine-1-carboxylate was separated by SFC (column: DAICEL CHIRALPAK AD (250 mm*50 mm, 10 um); mobile phase: [CO2-ACN/EtOH (0.1% IPAm)]; B %:45%, isocratic elution mode) to give the first fraction cis-benzyl 4-[3-(tert-butoxycarbonylamino)-3-methyl-cyclobutyl]piperazine-1-carboxylate (1.1 g, 61% yield) as a yellow solid (¹H NMR (400 MHz, CDCl₃) δ 7.30-7.23 (m, 5H), 5.05 (s, 2H), 4.63 (br s, 1H), 3.50-3.40 (m, 4H), 2.52-2.43 (m, 1H), 2.24-2.16 (m, 6H), 2.03-1.93 (m, 2H), 1.35 (s, 12H). LC-MS (ESI⁺) m/z 404.1 (M+H)+); and the second fraction trans-benzyl 4-[3-(tert-butoxycarbonylamino)-3-methyl-cyclobutyl]piperazine-1-carboxylate (0.45 g, 25% yield as a yellow solid (¹H NMR (400 MHz, CDCl₃) δ 7.29-7.22 (m, 5H), 5.06 (s, 2H), 3.47-3.42 (m, 4H), 2.79-2.69 (m, 1H), 2.32-2.14 (m, 6H), 1.82-1.74 (m, 2H), 1.37 (s, 9H), 1.34 (s, 3H). LC-MS (ESI⁺) m/z 404.0 (M+H)+). The absolute stereochemistry of the diastereomers was assigned by 2 D NMR.

### Synthesis of Benzyl 4-((1s,3s)-3-((tert-butoxycarbonyl)amino)-3-methylcyclobutyl)piperazine-1-carboxylate (Intermediate OH)

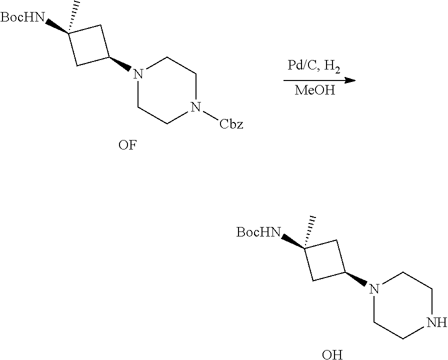 [SMILES: "MeOH" ; CC(C)(C)OC(=O)N[C@]1(C)C[C@H](N2CCN(C(=O)OCc3ccccc3)CC2)C1 ; "OF" ; CC(C)(C)OC(=O)N[C@]1(C)C[C@H](N2CCNCC2)C1 ; O]

To a solution of benzyl 4-((1s,3s)-3-((tert-butoxycarbonyl)amino)-3-methylcyclobutyl)piperazine-1-carboxylate (500 mg, 1.24 mmol, Intermediate OF) in MeOH (10 mL) was added Pd/C (500 mg, 469 mol, 10 wt %), then the mixture was stirred at 25° C. for 3 hrs under H₂ atmosphere (15 Psi). On completion, the reaction mixture was filtered and concentrated in vacuo to give the title compound (300 mg, 89% yield) as gray solid. ¹H NMR (400 MHz, DMSO-d₆) δ 6.94 (s, 1H), 3.23-3.13 (m, 1H), 2.29-2.06 (m, 6H), 2.05-1.76 (m, 6H), 1.36 (s, 10H), 1.28 (s, 3H). LC-MS (ESI⁺) m/z 270.2 (M+H)⁺.

### Synthesis of 3-(5-(4-((4-((1S,3s)-3-amino-3-methylcyclobutyl)piperazin-1-yl)methyl)piperidin-1-yl)-4-fluoro-3-methyl-2-oxo-2,3-dihydro-1H-benzo[d]imidazol-1-yl)piperidine-2,6-dione (Intermediate OI)

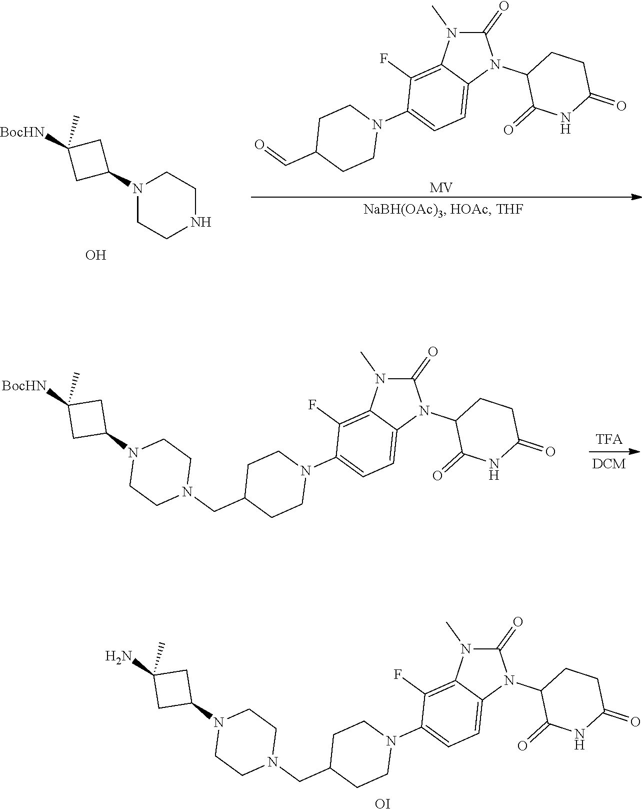

### Step 1—Tert-butyl((1s,3s)-3-(4-((1-(1-(2,6-dioxopiperidin-3-yl)-4-fluoro-3-methyl-2-oxo-2,3-dihydro -1H-benzo[d]imidazol-5-yl)piperidin-4-yl)methyl)piperazin-1-yl)-1-methylcyclobutyl)carbamate

To a solution of tert-butyl ((1s,3s)-1-methyl-3-(piperazin-1-yl)cyclobutyl)carbamate (124 mg, 460 mol, Intermediate OH) and 1-[1-(2,6-dioxo-3-piperidyl)-4-fluoro-3-methyl-2-oxo-benzimidazol-5-yl]piperidine-4-carbaldehyde (100 mg, 230 μmol, Intermediate MZ) in THF (2 mL) was added HOAc (230 mol, 13.1 μL) until the pH=4, then the mixture was stirred at 25° C. for 0.1 hr. Then, NaBH(OAc)₃ (73.1 mg, 345 μmol) was added and the mixture was stirred at 25° C. for 0.5 hr. On completion, the reaction mixture was quenched with H₂O (1 mL) at 0° C., the resulting mixture was filtered and the filtrate was concentrated under reduced pressure to give a residue. Then the residue was purified by reversed-phase (0.1% FA condition) to give the title compound (80.0 mg, 54% yield) as white solid. ¹H NMR (400 MHz, DMSO-d₆) δ 8.23 (s, 1H), 7.00-6.89 (m, 1H), 6.84 (d, J=8.8 Hz, 1H), 6.78-6.68 (m, 1H), 5.32 (dd, J=5.2, 12.8 Hz, 1H), 3.46 (s, 3H), 2.91-2.86 (m, 1H), 2.38-2.22 (m, 6H), 2.22-2.12 (m, 4H), 2.07-1.97 (m, 4H), 1.95-1.88 (m, 6H), 1.83-1.73 (m, 3H), 1.65-1.56 (m, 1H), 1.37 (s, 10H), 1.28 (s, 3H), 1.23 (s, 2H). LC-MS (ESI⁺) m/z 642.3 (M+H)⁺.

### Step 2—3-(5-(4-((4-((1S,3s)-3-amino-3-methylcyclobutyl)piperazin-1-yl)methyl)piperidin-1-yl)-4-fluoro-3-methyl-2-oxo-2,3-dihydro-1H-benzo[d]imidazol-1-yl)piperidine-2,6-dione

To a solution of tert-butyl ((1s,3s)-3-(4-((1-(1-(2,6-dioxopiperidin-3-yl)-4-fluoro-3-methyl-2-oxo-2,3-dihydro-1H-benzo[d]imidazol-5-yl)piperidin-4-yl)methyl)piperazin-1-yl)-1-methylcyclobutyl)carbamate (80.0 mg, 124 μmol) in DCM (1 mL) was added TFA (124 μmol, 9.26 μL), then the mixture was stirred at 25° C. for 0.5 hr. On completion, the reaction mixture was concentrated in vacuo to give the title compound (70.0 mg, 85% yield) as a yellow oil. LC-MS (ESI⁺) m/z 542.3 (M+H)⁺.

### Synthesis of Tert-butyl ((1r,3r)-1-methyl-3-(piperazin-1-yl)cyclobutyl)carbamate (Intermediate OJ)

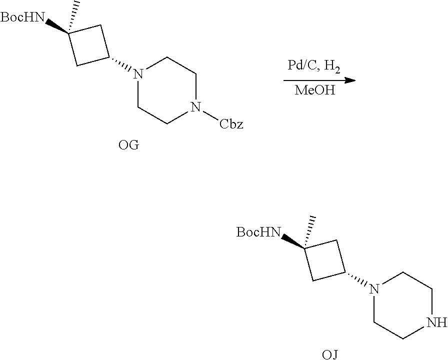

To a solution of benzyl 4-[3-(tert-butoxycarbonylamino)-3-methyl-cyclobutyl]piperazine-1-carboxylate (450 mg, 1.12 mmol, Intermediate OG) in MeOH (20 mL) was added Pd/C (4.50 g, 4.23 mmol, 10 wt %) under Ar atmosphere, and then the mixture was stirred at 15° C. for 1 hr under H₂ atmosphere (50 psi). On completion, the reaction mixture filtered and concentrated in vacuo to give the title compound (220 mg, 73% yield) as a black solid. LC-MS (ESI⁺) m/z 270.4 (M+H)⁺.

### Synthesis of 3-(5-(4-((4-((1r,3r)-3-amino-3-methylcyclobutyl)piperazin-1-yl)methyl)piperidin-1-yl)-4-fluoro-3-methyl-2-oxo-2,3-dihydro-1H-benzo[d]imidazol-1-yl)piperidine-2,6-dione (Intermediate OK)

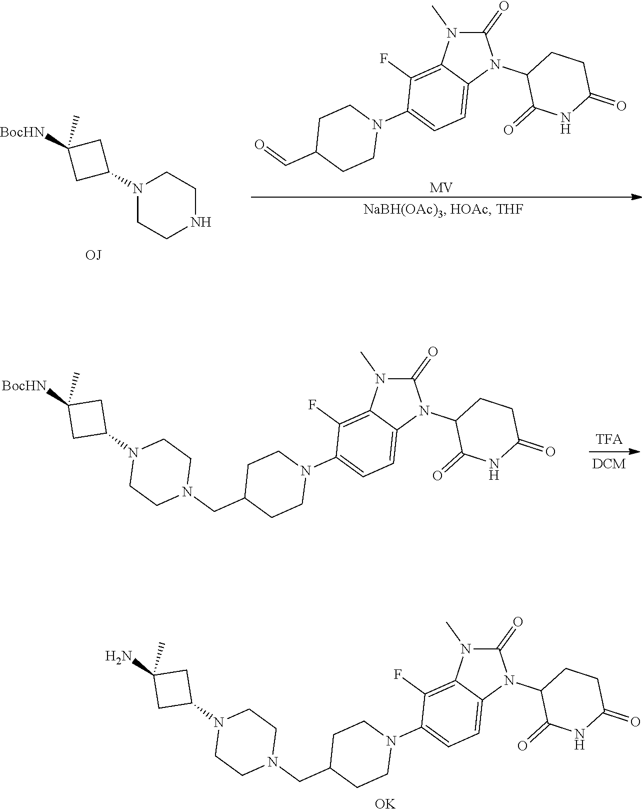

### Step 1—Tert-butyl ((1r,3r)-3-(4-((1-(1-(2,6-dioxopiperidin-3-yl)-4-fluoro-3-methyl-2-oxo-2,3-dihydro-1H-benzo[d]imidazol-5-yl)piperidin-4-yl)methyl)piperazin-1-yl)-1-methylcyclobutyl)carbamate

To a solution of cis-tert-butyl N-(1-methyl-3-piperazin-1-yl-cyclobutyl)carbamate (162 mg, 603 mol, Intermediate OJ) in THF (5 mL) was added and HOAc (20.1 mg, 334 μmol) and 1-[1-(2,6-dioxo-3-piperidyl)-4-fluoro-3-methyl-2-oxo-benzimidazol-5-yl]piperidine-4-carbaldehyde (130 mg, 334 μmol, Intermediate MZ) and the reaction mixture was stirred at 25° C. for 0.5 hr. Then NaBH(OAc)₃ (177 mg, 836 mol) was added, and the resulting mixture was stirred at 25° C. for another 1.5 hr. On completion, the reaction mixture filtered and concentrated in vacuo to give the residue. The residue was purified by reserve column (0.1% FA) to give the title compound (210.0 mg, 98% yield) as a black oil. ¹H NMR (400 MHz, CD₃OD-d₄) δ 8.33 (br s, 1H), 6.77-6.64 (m, 1H), 5.45-5.11 (m, 1H), 3.62-3.34 (m, 3H), 3.31-3.24 (m, 1H), 2.98-2.53 (m, 8H), 2.52-2.19 (m, 4H), 2.15-1.50 (m, 5H), 1.46-1.22 (m, 12H). LC-MS (ESI⁺) m z 642.4 (M+H)⁺.

### Step 2-3-(5-(4-((4-((1r,3r)-3-amino-3-methylcyclobutyl)piperazin-1-yl)methyl)piperidin-1-yl)-4-fluoro-3-methyl-2-oxo-2,3-dihydro-1H-benzo[d]imidazol-1-yl)piperidine-2,6-dione

To a solution of tert-butyl N-[3-[4-[[1-[1-(2,6-dioxo-3-piperidyl)-4-fluoro-3-methyl-2-oxo-benzimidazol-5-yl]-4-piperidyl]methyl]piperazin-1-yl]-1-methyl-cyclobutyl]carbamate (130 mg, 202 mol) in DCM (4 mL) was added TFA (2 mL), and then the mixture was stirred at 15° C. for 1 hr. On completion, the reaction mixture concentrated in vacuo to give the title compound (128 mg, 96% yield) as a black solid. LC-MS (ESI⁺) m/z 542.3 (M+H)⁺.

### Synthesis of 3-(3-Methyl-4-piperazin-1-yl-anilino)piperidine-2,6-dione (Intermediate OL)

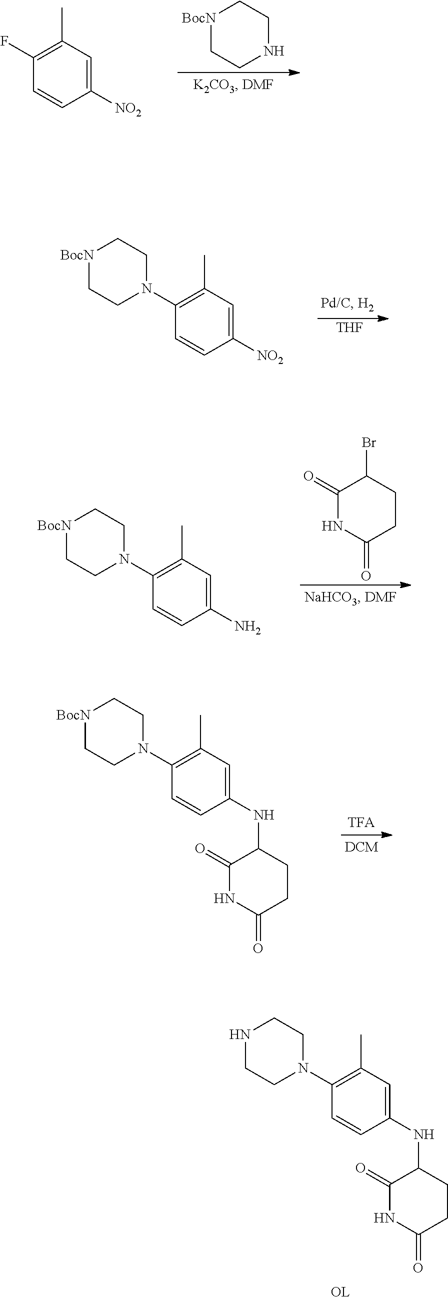

### Step 1—Tert-butyl 4-(2-methyl-4-nitro-phenyl)piperazine-1-carboxylate

To a solution of 1-fluoro-2-methyl-4-nitro-benzene (2.00 g, 12.89 mmol) and tert-butyl piperazine-1-carboxylate (2.88 g, 15.5 mmol) in DMF (40 mL) was added K₂CO₃ (1.78 g, 12.9 mmol), then the mixture was stirred at 80° C. for 24 hrs. On completion, the mixture was filtered, diluted with water (60 mL) and extracted with PE (50 mL). The combined organic layers with dried over anhydrous Na₂SO₄, filtered and concentrated in vacuo to give the residue. The residue was purified by column chromatography (SiO₂, PE:EA=50:1 to 30:1) to give the title product (2.83 g, 68% yield) as yellow solid. ¹H NMR (400 MHz, DMSO-d₆) δppm 7.53-7.61 (m, 2H) 6.68 (d, J=8.80 Hz, 1H) 3.03 (s, 4H) 2.79-2.92 (s, 4H) 1.89 (s, 3H) 0.97 (s, 9H). LC-MS (ESI⁺) m/z 266.0 (M−56)⁺.

### Step 2—Tert-butyl 4-[2-[4-[1-(2,6-dioxo-3-piperidyl)-4-fluoro-3-methyl-2-oxo-benzimidazol-5-yl]piperazin-1-yl]ethyl]piperidine-1-carboxylate

To a solution of tert-butyl 4-(2-methyl-4-nitro-phenyl)piperazine-1-carboxylate (1.40 g, 4.36 mmol) in THF (50 mL) was added Pd/C (1.00 g, 10 wt %) and purged with H₂ for three times, then the mixture was stirred at 25° C. for 2 hrs. On completion, the mixture was concentrated in vacuo to give the title compound (1.10 g, 87% yield) as yellow solid. LC-MS (ESI⁺) m/z 292.2 (M+H)⁺.

### Step 3—Tert-butyl 4-[4-[(2,6-dioxo-3-piperidyl)amino]-2-methyl-phenyl]piperazine-1-carboxylate

To a solution of tert-butyl 4-(4-amino-2-methyl-phenyl)piperazine-1-carboxylate (500 mg, 1.72 mmol) and 3-bromopiperidine-2,6-dione (494 mg, 2.57 mmol) in DMF (10 mL) was added NaHCO₃ (432 mg, 5.15 mmol), then the mixture was stirred at 65° C. for 16 hos. On completion, the reaction mixture was added ice-water (100 mL) and stirred for 5 minutes, the sediment was filtered through the buchner funnel to obtain the solid title product (670 mg, 97% yield) as black solid. ¹H NMR (400 MHz, DMSO-d₆) δ ppm 10.58 (s, 1H) 6.66 (d, J=8.40 Hz, 1H) 6.37 (s, 1H) 6.31 (d, J=7.24 Hz, 1H) 5.32 (d, J=7.12 Hz, 1H) 3.26 (s, 4H) 2.73 (s, 2H) 2.58 (s, 2H) 2.35 (s, 2H) 2.01 (s, 3H) 1.94 (m, J=12.4, 3.13 Hz, 1H) 1.61-1.75 (m, 1H) 1.26 (s, 9H). LC-MS (ESI⁺) m/z 403.1 (M+H)⁺.

### Step 4—3-(3-Methyl-4-piperazin-1-yl-anilino)piperidine-2,6-dione

To a solution of tert-butyl 4-[4-[(2, 6-dioxo-3-piperidyl)amino]-2-methyl-phenyl]piperazine-1-carboxylate (300 mg, 745 umol) in DCM (4 mL) was added TFA (1 mL), then the mixture was stirred at 25° C. for 1 hr. On completion, the mixture was concentrated in vacuo to give the title compound (180 mg, 79% yield) as brown solid. LC-MS (ESI⁺) m/z 303.3 (M+H)⁺.

### Synthesis of 3-[4-[4-[[4-[(2R)-2-aminopropoxy]cyclohexyl]methyl]piperazin-1-yl]-3-methyl-anilino]piperidine-2,6-dione (Intermediate OM)

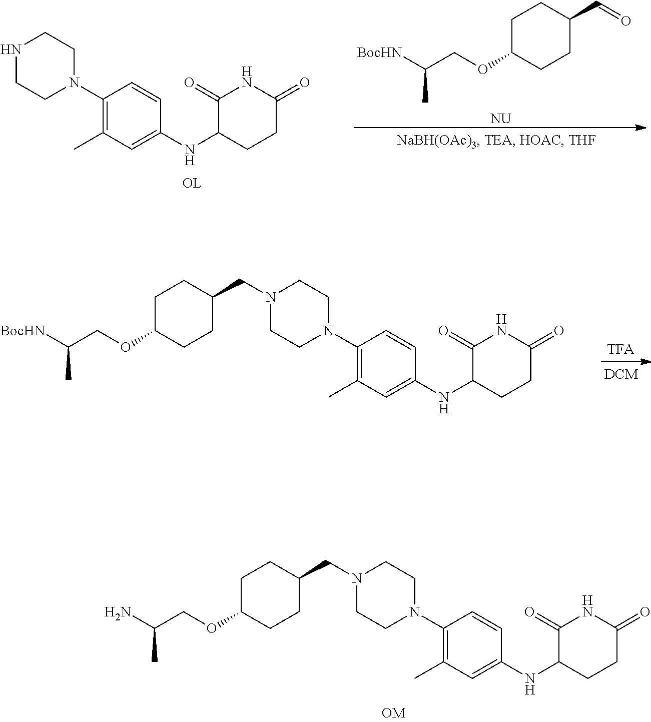 [SMILES: Cc1cc(NC2CCC(=O)NC2=O)ccc1N1CCNCC1 ; C[C@H](CO[C@H]1CC[C@H](C=O)CC1)NC(=O)OC(C)(C)C ; "NU" ; Cc1cc(NC2CCC(=O)NC2=O)ccc1N1CCN(C[C@H]2CC[C@H](OC[C@@H](C)NC(=O)OC(C)(C)C)CC2)CC1 ; Cc1cc(NC2CCC(=O)NC2=O)ccc1N1CCN(C[C@H]2CC[C@H](OC[C@@H](C)N)CC2)CC1]

### Step 1—Tert-butyl N-[(1R)-2-[4-[[4-[4-[(2,6-dioxo-3-piperidyl)amino]-2-methyl-phenyl]piperazin-1-yl]methyl]cyclohexoxy]-1-methyl-ethyl]carbamate

To a solution of 3-(3-methyl-4-piperazin-1-yl-anilino)piperidine-2,6-dione (185 mg, 444 μmol, Intermediate OL) in THF (1 mL) was added TEA (44.9 mg, 444 μmol) until pH=8-10. Then tert-butyl N-[(1R)-2-(4-formylcyclohexoxy)-1-methyl-ethyl]carbamate (200 mg, 700 μmol, Intermediate NU) in DMF (0.1 mL) and HOAc (26.7 mg, 444 μmol) was added until pH=4-5, then the mixture was stirred at −10° C. for 0.5 hr. Next, NaBH(OAc)₃ (188 mg, 888 μmol) was added to the mixture and the mixture was stirred at −10° C. for 0.5 hr. On completion, the mixture was filtered and concentrated in vacuo to give the residue. The residue was purified by prep-HPLC (column: Welch Xtimate C18 150*25 mm*5 um; mobile phase: [water (FA)-ACN]; gradient: 12%-42% B) to give the title compound (130 mg, 51% yield) as red solid. ¹H NMR (400 MHz, DMSO-d₆) δ 10.75 (s, 1H), 6.83 (d, J=8.4 Hz, 1H), 6.59 (d, J=7.2 Hz, 1H), 6.53-6.47 (m, 1H), 5.75 (s, 1H), 5.49 (d, J=7.2 Hz, 1H), 3.55-3.50 (m, 1H), 3.38-3.33 (m, 6H), 3.20-3.10 (m, 3H), 2.78 (s, 3H), 2.71-2.65 (m, 2H), 2.59-2.48 (m, 1H), 2.54 (d, J=4.4 Hz, 1H), 2.14 (s, 3H), 2.11-2.05 (m, 1H), 1.95 (d, J=9.6 Hz, 2H), 1.79 (d, J=12.0 Hz, 2H), 1.53 (s, 1H), 1.37 (s, 9H), 1.26-1.19 (m, 1H), 1.14-1.06 (m, 2H), 0.99 (d, J=6.4 Hz, 3H), 0.93-0.84 (m, 2H). LC-MS (ESI⁺) m/z 572.2 (M+H)⁺.

### Step 2—3-[4-[4-[[4-[(2R)-2-aminopropoxy]cyclohexyl]methyl]piperazin-1-yl]-3-methyl-anilino]piperidine-2,6-dione

To a solution of tert-butyl N-[(1R)-2-[4-[[4-[4-[(2,6-dioxo-3-piperidyl)amino]-2-methyl-phenyl]piperazin-1-yl]methyl]cyclohexoxy]-1-methyl-ethyl]carbamate (100 mg, 174 μmol) in DCM (2.0 mL) was added TFA (590 mg, 5.18 mmol), then the mixture was stirred at 25° C. for 0.5 hr. On completion, the mixture was concentrated in vacuo to give the title compound (100 mg, 97% yield) as black oil. LC-MS (ESI⁺) m/z 472.1 (M+H)⁺.

### Synthesis of 4-[[7′-[(1R,3R)-3-[tert-butyl(diphenyl)silyl]oxycyclohexyl]-6′-oxo-spiro[cyclopropane-1,5′-pyrrolo[2,3-d]pyrimidine]-2′-yl]amino]-3-methyl-benzenesulfonyl chloride (Intermediate ON)

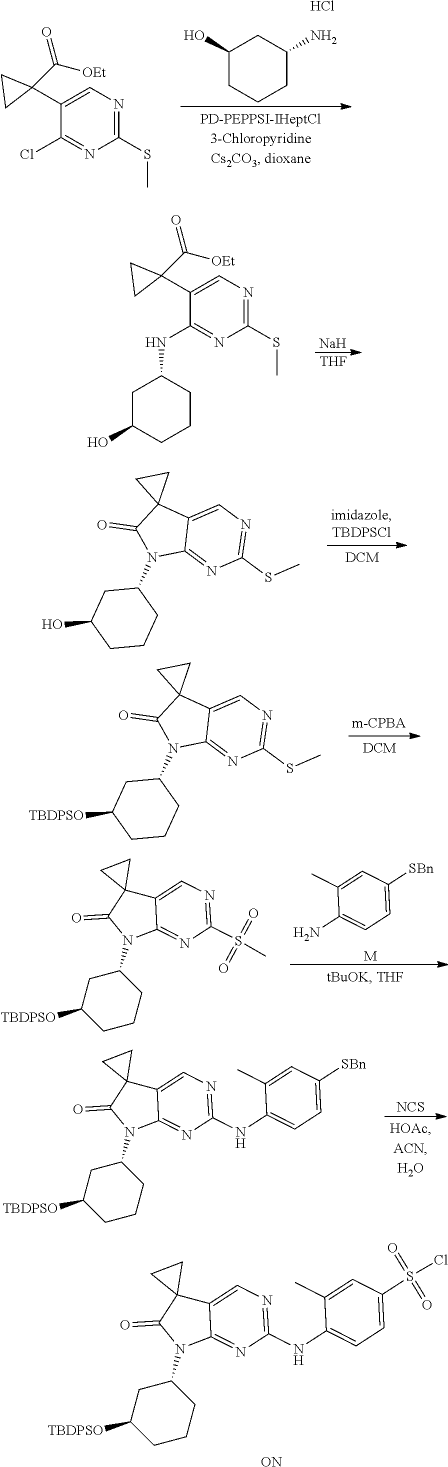

### Step 1—Ethyl 1-[4-[[(1R,3R)-3-hydroxycyclohexyl]amino]-2-methylsulfanyl-pyrimidin-5-yl]cyclopropanecarboxylate

To a mixture of ethyl 1-(4-chloro-2-methylsulfanyl-pyrimidin-5-yl)cyclopropanecarboxylate (2.50 g, 9.17 mmol, synthesized via Step 1 of Intermediate NP) and (1R,3R)-3-aminocyclohexanol hydrochloride (2.59 g, 13.7 mmol, HCl, CAS #1817645-57-0) in dioxane (30 mL) was added Cs₂CO₃ (8.96 g, 27.5 mmol) and 1,3-bis[2,6-bis(1-propylbutyl)phenyl]-4,5-dichloro-2H-imidazol-1-ium-2-ide 3-chloropyridine; dichloropalladium (891 mg, 916 μmol) under N₂. The reaction mixture was stirred at 100° C. for 12 hrs. On completion, the residue was diluted with water (20 mL), then the residue was extracted with EA (3×50 mL). The combined organic layers were dried over Na₂SO₄, filtered and filtrate was concentrated in vacuo. The residue was purified by column chromatography (SiO₂, PE:EA=50:1 to PE:EA=5:1) to give the title compound (3.40 g, 52% yield) as red solid. ¹H NMR (400 MHz, DMSO-d₆) δ 8.08 (s, 1H), 5.75 (s, 1H), 4.67 (ddd, J=3.6, 8.8, 12.4 Hz, 1H), 4.58 (d, J=2.8 Hz, 1H), 4.48-4.37 (m, 1H), 4.10 (d, J=2.0 Hz, 1H), 4.06-3.92 (m, 1H), 2.48-2.38 (m, 2H), 2.21 (dq, J=3.6, 12.8 Hz, 1H), 1.86-1.73 (m, 4H), 1.72-1.62 (m, 4H), 1.62-1.49 (m, 5H), 1.42-1.27 (m, 2H).

### Step 2—7′-[(1R,3R)-3-hydroxycyclohexyl]-2′-methylsulfanyl-spiro[cyclopropane-1,5′-pyrrolo[2,3-d]pyrimidine]-6′-one

To a solution of ethyl 1-[4-[[(1R,3R)-3-hydroxycyclohexyl]amino]-2-methylsulfanyl-pyrimidin-5-yl] cyclopropanecarboxylate (4.30 g, 12.2 mmol) in THF (50 mL) was added NaH (978 mg, 24.4 mmol, 60% dispersion in mineral oil) at 0° C. The reaction mixture was stirred at 25° C. for 1 hr. On completion, the residue was quenched with NH₄Cl (5 mL), diluted with water (20 mL), then the residue was extracted with EA (3×50 mL). The combined organic layers were dried over Na₂SO₄, filtered and filtrate was concentrated in vacuo to give the title compound (3.70 g, 99% yield) as red solid. ¹H NMR (400 MHz, DMSO-d₆) δ 8.10-8.04 (m, 1H), 5.75 (s, 1H), 4.73-4.63 (m, 1H), 4.58 (d, J=2.4 Hz, 1H), 4.11 (s, 1H), 2.47-2.40 (m, 1H), 2.26-2.16 (m, 1H), 1.99 (s, 1H), 1.80-1.73 (m, 3H), 1.71-1.51 (m, 7H), 1.43-1.31 (m, 1H). LC-MS (ESI⁺) m/z 306.2 (M+H).

### Step 3—7′-[(1R,3R)-3-[tert-butyl(diphenyl)silyl]oxycyclohexyl]-2′-methylsulfanyl-spiro[cyclopropane-1,5′-pyrrolo[2,3-d]pyrimidine]-6′-one

To a mixture of 7′-[(1R,3R)-3-hydroxycyclohexyl]-2′-methylsulfanyl-spiro[cyclopropane-1,5′-pyrrolo [2,3-d]pyrimidine]-6′-one (3.70 g, 12.1 mmol) in DCM (5 mL) was added TBDPSCl (5.00 g, 18.1 mmol) and imidazole (2.47 g, 36.3 mmol). The reaction mixture was then stirred at 25° C. for 1 hr. On completion, the residue was diluted with water (20 mL), then the residue was extracted with EA (3×50 mL). The combined organic layer was dried over Na₂SO₄, filtered and filtrate was concentrated in vacuo. The residue was purified by column chromatography (SiO₂, PE:EA=50:1 to PE:EA=5:1) to give the title compound (4 g, 60% yield) as red solid. ¹H NMR (400 MHz, DMSO-d₆) δ 8.11-8.06 (m, 1H), 7.62 (d, J=6.4 Hz, 4H), 7.48-7.31 (m, 6H), 5.00-4.86 (m, 1H), 4.25 (s, 1H), 2.47 (s, 3H), 2.37-2.19 (m, 2H), 2.00-1.84 (m, 1H), 1.81-1.72 (m, 3H), 1.67-1.54 (m, 5H), 1.31 (t, J=12.4 Hz, 1H), 1.06 (s, 9H); LC-MS (ESI⁺) m/z 544.9 (M+H).

### Step 4—7′-[(1R,3R)-3-[tert-butyl(diphenyl)silyl]oxycyclohexyl]-2′-methylsulfonyl-spiro[cyclopropane-1,5′-pyrrolo[2,3-d]pyrimidine]-6′-one

To a mixture of 7′-[(1R,3R)-3-[tert-butyl(diphenyl)silyl]oxycyclohexyl]-2′-methylsulfanyl-spiro [cyclopropane-1,5′-pyrrolo[2,3-d]pyrimidine]-6′-one (3.50 g, 6.44 mmol) in DCM (5 mL) was added m-CPBA (5.23 g, 25.7 mmol, 85% solution). The reaction mixture was then stirred at 25° C. for 1 hr. On completion, the reaction mixture was quenched by saturated Na₂SO₃ (50 mL) at 25° C., and then stirred for 30 minutes. The mixture was extracted with DCM (2×100 mL) then the combined organic layers were dried over Na₂SO₄, filtered and concentrated in vacuo. The residue was purified by column chromatography (SiO₂, PE:EA=10:1 to PE:EA=1:1) to give the title compound (3.30 g, 89% yield) as white solid. ¹H NMR (400 MHz, DMSO-d₆) δ 8.44 (s, 1H), 7.64-7.60 (m, 4H), 7.48-7.33 (m, 6H), 5.01-4.90 (m, 1H), 4.28 (s, 1H), 3.34 (s, 3H), 2.38-2.31 (m, 1H), 2.28-2.20 (m, 1H), 2.00 (d, J=3.6 Hz, 2H), 1.92 (dd, J=3.2, 16.4 Hz, 1H), 1.82-1.74 (m, 3H), 1.70-1.57 (m, 3H), 1.40-1.30 (m, 1H), 1.06 (s, 9H). LC-MS (ESI⁺) m/z 576.2 (M+H).

### Step 5—2′-(4-Benzylsulfanyl-2-methyl-anilino)-7′-[(1R,3R)-3-[tert-butyl(diphenyl)silyl]oxycyclohexyl]spiro[cyclopropane-1,5′-pyrrolo[2,3-d]pyrimidine]-6′-one

To a mixture of 7′-[(1R,3R)-3-[tert-butyl(diphenyl)silyl]oxycyclohexyl]-2′-methylsulfonyl-spiro [cyclopropane-1,5′-pyrrolo[2,3-d]pyrimidine]-6′-one (500 mg, 868 μmol) and 4-benzylsulfanyl -2-methyl-aniline (199 mg, 868 μmol, Intermediate M) in DMF (1 mL) was added 4A molecular sieves (10.0 mg, 868 mol) and t-BuOK (292 mg, 2.61 mmol). The reaction mixture was stirred at 25° C. for 1 hr. On completion, the residue was diluted with water (10 mL), then the residue was extracted with EA (3×30 mL). The combined organic layers were dried over Na₂SO₄, filtered and filtrate was concentrated in vacuo. The residue was purified by column chromatography (SiO₂, PE:EA=50:1 to PE:EA=1:1) and prep-HPLC (column: Phenomenex Luna C18 150*30 mm*5 um; mobile phase: [water (HCl)-ACN]; gradient:70%-100% B over 10 min) to give the title compound (200 mg, 15% yield) as yellow solid. ¹H NMR (400 MHz, DMSO-d₆) δ 10.03-9.72 (m, 1H), 7.93 (s, 1H), 7.63-7.54 (m, 4H), 7.50-7.34 (m, 7H), 7.33-7.28 (m, 2H), 7.27-7.20 (m, 3H), 7.16 (t, J=7.2 Hz, 2H), 4.82 (t, J=12.4 Hz, 1H), 4.17 (s, 3H), 2.18 (s, 4H), 2.11-2.00 (m, 1H), 1.88-1.75 (m, 3H), 1.71-1.61 (m, 3H), 1.59-1.50 (m, 3H), 1.02 (s, 9H); LC-MS (ESI⁺) m/z 725.8 (M+H).

### Step 6—4-[[7′-[(1R,3R)-3-[tert-butyl(diphenyl)silyl]oxycyclohexyl]-6′-oxo-spiro[cyclopropane-1,5′-pyrrolo[2,3-d]pyrimidine]-2′-yl]amino]-3-methyl-benzenesulfonyl chloride

To a solution of 2′-(4-benzylsulfanyl-2-methyl-anilino)-7′-[(1R,3R)-3-[tert-butyl(diphenyl)silyl]oxycyclohexyl]spiro[cyclopropane-1,5′-pyrrolo[2,3-d]pyrimidine]-6′-one (60.0 mg, 82.7 μmol) in ACN (2 mL) and AcOH (0.2 mL) was added H₂O (1.49 mg, 82.7 mol) and NCS (33.1 mg, 248 mol). The reaction mixture was stirred at 20° C. for 0.5 hr in the dark. On completion, the mixture was diluted with EA (20 mL) and washed with water (10 mL×3). The organic layer was dried over Na₂SO₄, filtered and the filtrate was concentrated in vacuo to give the title compound (58 mg, 99% yield) as colorless oil. LC-MS (ESI⁺) m/z 701.1 (M+H)⁺.

### Synthesis of 3-[4-[4-[(4-Aminocyclohexyl)methyl]piperazin-1-yl]-3-methoxy-phenyl]piperidine-2,6-dione (Intermediate 00)

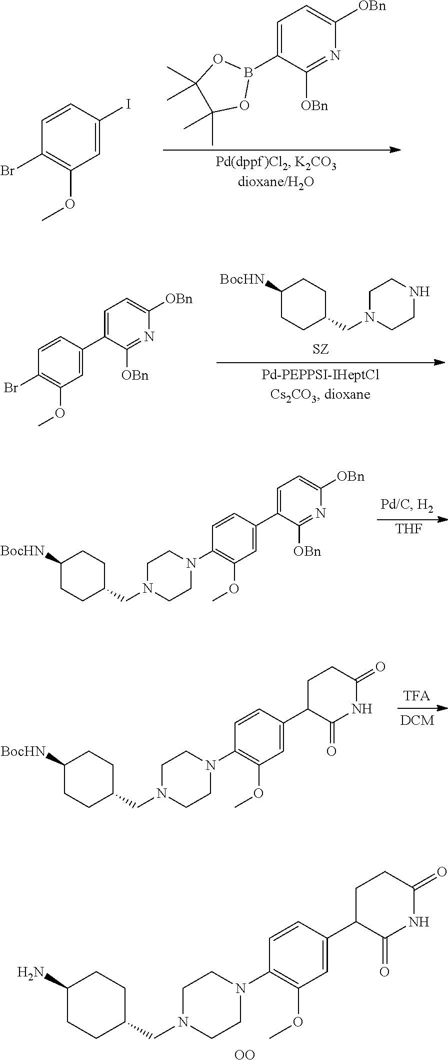

### Step 1—2,6-Dibenzyloxy-3-(4-bromo-3-methoxy-phenyl)pyridine

A mixture of 1-bromo-4-iodo-2-methoxy-benzene (4.9 g, 15.6 mmol, CAS #755027-18-0), 2,6-dibenzyloxy-3-(4,4,5,5-tetramethyl-1,3,2-dioxaborolan-2-yl)pyridine (5.23 g, 12.5 mmol, CAS #2152673-80-6), Pd(dppf)Cl₂·CH₂Cl₂ (1.28 g, 1.57 mmol), and K₂CO₃ (6.49 g, 46.9 mmol) in dioxane (80 mL) and H₂O (16 mL) was stirred at 80° C. for 2 hrs. On completion, the reaction was diluted with EA (100 mL). The organic layer was washed with water (100 mL), dried over Na₂SO₄ and concentrated in vacuo. The residue was purified by column chromatography (SiO₂, Petroleum ether/Ethyl acetate=I/O to 5/1) to give the title compound (5.2 g, 69% yield) as yellow solid. ¹H NMR (400 MHz, CDCl₃) δ 7.55 (d, J=8.0 Hz, 1H), 7.45 (d, J=8.0 Hz, 1H), 7.40-7.36 (m, 2H), 7.33-7.22 (m, 8H), 7.10 (d, J=2.0 Hz, 1H), 6.91 (dd, J=2.0, 8.0 Hz, 1H), 6.42 (d, J=8.0 Hz, 1H), 5.33 (s, 4H), 3.67 (s, 3H).

### Step 2—Tert-butyl N-[4-[[4-[4-(2,6-dibenzyloxy-3-pyridyl)-2-methoxy-phenyl]piperazin-1-yl]methyl]cyclohexyl]carbamate

A mixture of 2,6-dibenzyloxy-3-(4-bromo-3-methoxy-phenyl)pyridine (500 mg, 1.05 mmol), tert-butyl N-[4-(piperazin-1-ylmethyl)cyclohexyl]carbamate (468 mg, 1.57 mmol, Intermediate SZ), Cs₂CO₃ (1.03 g, 3.15 mmol), and 1,3-bis[2,6-bis(1-propylbutyl)phenyl]-4,5-dichloro-2H-imidazol-1-ium-2-ide 3-chloropyridine dichloropalladium (102 mg, 104 μmol) in dioxane (10 mL) was stirred at 110° C. for 16 hrs under N₂. On completion the reaction was diluted with EA (50 mL). The organic layer was washed with water (50 mL), dried over Na₂SO₄ and concentrated in vacuo. The residue was purified by column chromatography (SiO₂, DCM/Ethyl acetate=10/1 to 1/1) to give the title compound (460 mg, 63% yield) as yellow oil. ¹H NMR (400 MHz, CDCl₃) δ 7.69-7.61 (m, 1H), 7.50-7.30 (m, 11H), 7.18 (s, 1H), 7.14-7.04 (m, 1H), 6.96 (d, J=8.4 Hz, 1H), 6.49 (d, J=8.0 Hz, 1H), 5.42 (d, J=8.0 Hz, 4H), 4.49-4.31 (m, 1H), 3.73 (s, 3H), 3.49-3.33 (m, 1H), 3.23-3.04 (m, 4H), 2.71-2.53 (m, 4H), 2.29-2.18 (m, 2H), 2.09-2.03 (m, 2H), 1.95-1.86 (m, 2H), 1.47 (s, 10H), 1.17-0.99 (m, 4H).

### Step 3—Tert-butyl N-[4-[[4-[4-(2,6-dioxo-3-piperidyl)-2-methoxy-phenyl]piperazin-1-yl]methyl]cyclohexyl]carbamate

To a solution of tert-butyl N-[4-[[4-[4-(2,6-dibenzyloxy-3-pyridyl)-2-methoxy-phenyl]piperazin-1-yl] methyl]cyclohexyl]carbamate (460 mg, 663 μmol) in THF (10 mL) was added Pd/C (300 mg, 281 mol, 10 wt %) under N₂. The suspension was degassed under vacuum and purged with H₂ several times. The mixture was stirred under H₂ (15 psi) at 25° C. for 16 hrs. On completion, the reaction was filtered and filtrate was concentrated in vacuo to give the title compound (330 mg, 96% yield) as yellow solid. ¹H NMR (400 MHz, DMSO-d₆) δ 10.79 (s, 1H), 6.89-6.78 (m, 2H), 6.74-6.63 (m, 2H), 3.79-3.74 (m, 3H), 3.31 (s, 1H), 3.22-3.07 (m, 1H), 2.94 (s, 3H), 2.70-2.60 (m, 1H), 2.45 (d, J=4.0 Hz, 4H), 2.25-2.17 (m, 1H), 2.16-2.08 (m, 2H), 2.07-1.97 (m, 1H), 1.77 (d, J=10.8 Hz, 4H), 1.38 (s, 9H), 1.36 (s, 2H), 1.18-1.06 (m, 2H), 0.93-0.81 (m, 2H).

### Step 4—3-[4-[4-[(4-Aminocyclohexyl)methyl]piperazin-1-yl]-3-methoxy-phenyl]piperidine-2,6-dione

A mixture of tert-butyl N-[4-[[4-[4-(2,6-dioxo-3-piperidyl)-2-methoxy-phenyl]piperazin-1-yl]methyl] cyclohexyl]carbamate (100 mg, 194 μmol) in TFA (460 mg, 4.04 mmol, 0.3 mL) and DCM (1 mL) was stirred at 25° C. for 1 hr. On completion the reaction was concentrated in vacuo to give the title compound (102 mg, 99% yield, TFA) as yellow oil. LC-MS (ESI⁺) m/z 415.2 (M+H)⁺.

### Synthesis of 3-(3-Fluoro-4-piperazin-1-yl-phenyl)piperidine-2,6-dione (Intermediate OP)

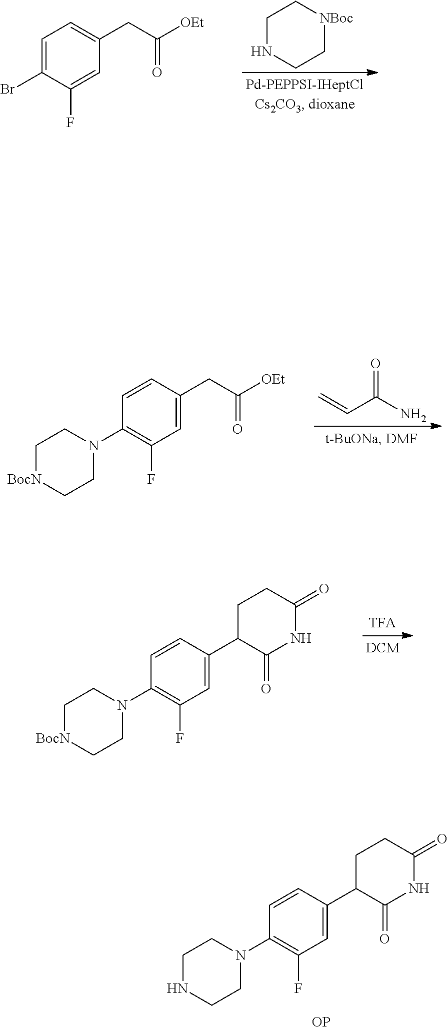

### Step 1—Tert-butyl 4-[4-(2-ethoxy-2-oxo-ethyl)-2-fluoro-phenyl]piperazine-1-carboxylate

To a solution of tert-butyl piperazine-1-carboxylate (2.85 g, 15.3 mmol, CAS #143238-38-4) and ethyl 2-(4-bromo-3-fluoro-phenyl)acetate (2.00 g, 7.66 mmol, CAS #1296223-82-9) in dioxane (20 mL) were added Cs₂CO₃ (7.49 g, 22.9 mmol) and Pd-PEPPSI-IHeptCl (745 mg, 766 μmol), then the mixture was stirred at 100° C. for 10 hrs. On completion, the mixture was filtered and the filtrate was concentrated in vacuo to give the residue. The residue was purified by column chromatography (SiO₂, PE:EA=20:1 to 10:1) to give the title compound (2.50 g, 89% yield) as yellow oil. ¹H NMR (400 MHz, DMSO-d₆) δ 7.10-7.04 (m, 1H), 7.02-6.95 (m, 2H), 4.07 (q, J=7.2 Hz, 2H), 3.60 (s, 2H), 3.46 (s, 4H), 2.96-2.88 (m, 4H), 1.42 (s, 9H), 1.18 (t, J=7.2 Hz, 3H). LC-MS (ESI⁺) m/z 367.0 (M+H)⁺.

### Step 2—Tert-butyl 4-[4-(2,6-dioxo-3-piperidyl)-2-fluoro-phenyl]piperazine-1-carboxylate

To a solution of tert-butyl 4-[4-(2-ethoxy-2-oxo-ethyl)-2-fluoro-phenyl]piperazine-1-carboxylate (2.00 g, 5.46 mmol) and prop-2-enamide (1.94 g, 27.3 mmol, CAS #9003-05-8) in DMF (20 mL) was added tBuONa (524 mg, 5.46 mmol) slowly at −10° C., then the mixture was stirred at −10° C. for 1 hr under N₂. On completion, the mixture was quenched with sat. NH₄Cl (80 mL) and extracted with EA (40 mL×3). The combined organic layers were concentrated in vacuo to give the residue. The residue was diluted with EA and PE (60 mL, EA:PE=1:2) and stirred for 1 hr, then filtered to give the title compound (2.00 g, 93% yield) as light yellow solid. ¹H NMR (400 MHz, DMSO-d₆) δ 10.82 (s, 1H), 7.07-7.02 (m, 1H), 7.01-6.94 (m, 2H), 3.83-3.79 (m, 1H), 3.46 (s, 4H), 2.96-2.91 (m, 4H), 2.68-2.59 (m, 1H), 2.47-2.46 (m, 1H), 2.25-2.14 (m, 1H), 2.02-1.94 (m, 1H), 1.41 (s, 9H). LC-MS (ESI⁺) m/z 392.0 (M+H)⁺.

### Step 3—3-(3-Fluoro-4-piperazin-1-yl-phenyl)piperidine-2,6-dione

A solution of tert-butyl 4-[4-(2,6-dioxo-3-piperidyl)-2-fluoro-phenyl]piperazine-1-carboxylate (300 mg, 766 μmol) in DCM (3 mL) and TFA (1 mL) was stirred at 25° C. for 0.5 hr. On completion, the mixture was concentrated in vacuo to give the title compound (285 mg, 92% yield, TFA) as yellow liquid. LC-MS (ESI⁺) m/z 292.0 (M+H)⁺.

### Synthesis of 3-[4-[4-[(4-Aminocyclohexyl)methyl]piperazin-1-yl]-3-fluoro-phenyl]piperidine-2,6-dione (Intermediate OQ)

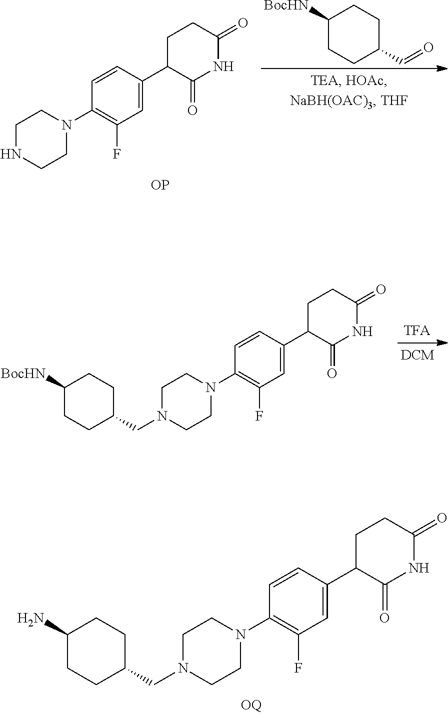

### Step 1—Tert-butyl N-[4-[[4-[4-(2,6-dioxo-3-piperidyl)-2-fluoro-phenyl]piperazin-1-yl]methyl]cyclohexyl]carbamate

To a solution of 3-(3-fluoro-4-piperazin-1-yl-phenyl)piperidine-2,6-dione (285 mg, 703 μmol, TFA, Intermediate OP) in THF (3 mL) was added TEA (196 μL, 1.41 mmol) and HOAc (40.2 μL, 703 mol) to adjust pH=6-7. Then tert-butyl N-(4-formylcyclohexyl)carbamate (160 mg, 703 μmol, CAS#181308-57-6) was added, and the mixture was stirred at −10° C. for 0.5 hr. Next, NaBH(OAc)₃ (223 mg, 1.05 mmol) was added, and the mixture was stirred at −10° C. for 1.5 hrs. On completion, the mixture was quenched with water (50 mL) and extracted with EA (30 mL×3). The combined organic layers were dried over anhydrous Na₂SO₄, filtered and concentrated in vacuo to give the title compound (250 mg, 70% yield) as yellow solid. ¹H NMR (400 MHz, DMSO-d₆) δ 10.84 (s, 1H), 9.52-9.08 (m, 1H), 7.09-6.97 (m, 2H), 6.84-6.63 (m, 1H), 3.84-3.80 (m, 1H), 3.66-3.44 (m, 2H), 3.20-3.04 (m, 6H), 2.70-2.60 (m, 1H), 2.25-2.13 (m, 1H), 2.02-1.97 (m, 1H), 1.79-1.76 (m, 5H), 1.37 (s, 13H), 1.16 (s, 2H), 1.06-0.89 (m, 2H). LC-MS (ESI⁺) m/z 503.1 (M+H)⁺.

### Step 2—3-[4-[4-[(4-Aminocyclohexyl)methyl]piperazin-1-yl]-3-fluoro-phenyl]piperidine-2,6-dione

A solution of tert-butyl N-[4-[[4-[4-(2,6-dioxo-3-piperidyl)-2-fluoro-phenyl]piperazin-1-yl]methyl]cyclohexyl]carbamate (80.0 mg, 159 μmol) in DCM (1 mL) and TFA (0.3 mL) was stirred at 25° C. for 0.5 hr. On completion, the mixture was concentrated in vacuo to give the title compound (78.0 mg, 95% yield, TFA) as yellow gum. LC-MS (ESI⁺) m/z 403.1 (M+H)⁺.

### Synthesis of 3-[4-[4-[(4-Amino-2-oxabicyclo[2.2.2]octan-1-yl)methyl]piperazin-1-yl]-2-chloro-phenyl] piperidine-2,6-dione (Intermediate OR)

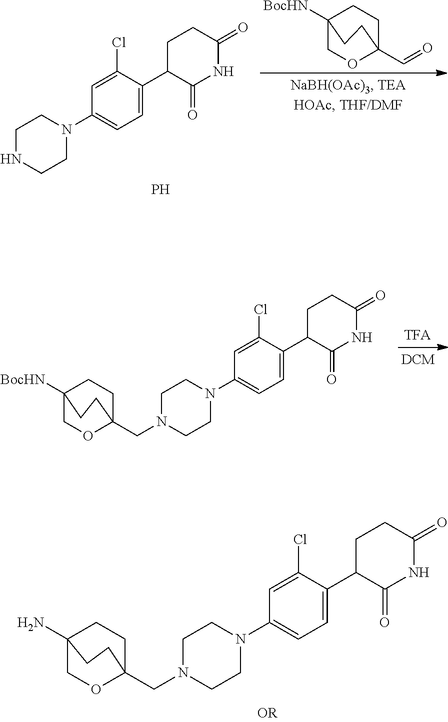

### Step 1—Tert-butyl N-[1-[[4-[3-chloro-4-(2,6-dioxo-3-piperidyl)phenyl]piperazin-1-yl]methyl]-2-oxabicyclo[2.2.2]octan-4-yl]carbamate

To a solution of 3-(2-chloro-4-piperazin-1-yl-phenyl)piperidine-2,6-dione (200 mg, 474 μmol, TFA, Intermediate PH) in THF (2 mL) and DMF (2 mL) was added TEA (718 μmol, 0.1 mL). Then tert-butyl N-(1-formyl-2-oxabicyclo[2.2.2]octan-4-yl)carbamate (121 mg, 474 μmol, CAS #1417551-42-8) and AcOH (1.75 mmol, 0.1 mL) was added and the mixture was stirred at 25° C. for 0.5 hr. Next, NaBH(OAc)₃ (150 mg, 711 μmol) was added to the former mixture, and the mixture was stirred 25° C. for 1 hr. On completion, the mixture was quenched with H₂O (0.5 mL), filtered and concentrated in vacuo to give the residue. The residue was purified by prep-HPLC (column: Waters xbridge 150*25 mm 10 um; mobile phase: [water (NH₄HCO₃)-ACN]; gradient: 37%-57% B over 14 min) to give the title compound (190 mg, 73% yield) as a white solid. ¹H NMR (400 MHz, DMSO-d₆) δ 10.86 (s, 1H), 7.19 (d, J=8.4 Hz, 1H), 7.04 (d, J=2.4 Hz, 1H), 6.94 (dd, J=2.4, 8.8 Hz, 1H), 6.73 (s, 1H), 4.08 (d, J=7.2 Hz, 1H), 3.84 (s, 2H), 3.84-3.75 (m, 2H), 3.66-3.58 (m, 2H), 3.26-3.12 (m, 6H), 2.81-2.69 (m, 1H), 2.56-2.48 (m, 1H), 2.28-2.20 (m, 1H), 2.02-1.89 (m, 5H), 1.85-1.78 (m, 2H), 1.73-1.64 (m, 2H), 1.36 (s, 9H). LC-MS (ESI⁺) m/z 547.1 (M+H)⁺.

### Step 2—3-[4-[4-[(4-Amino-2-oxabicyclo[2.2.2]octan-1-yl)methyl]piperazin-1-yl]-2-chloro-phenyl]piperidine-2,6-dione

To a solution of tert-butyl N-[1-[[4-[3-chloro-4-(2,6-dioxo-3-piperidyl)phenyl]piperazin-1-yl]methyl]-2-oxabicyclo[2.2.2]octan-4-yl]carbamate (100 mg, 182 μmol) in DCM (2 mL) was added TFA (0.5 mL), then the mixture was stirred at 25° C. for 1 hr. On completion, the mixture was concentrated in vacuo to give the title compound (100 mg, 97% yield, TFA) as a brown oil. LC-MS (ESI⁺) m/z 447.1 (M+H)⁺.

### Synthesis of 7′-cyclopentyl-2′-[2-methyl-4-(4-piperidylsulfonyl)anilino]spiro[cyclopropane -1,5′-pyrrolo[2,3-d]pyrimidine]-6′-one (Intermediate OS)

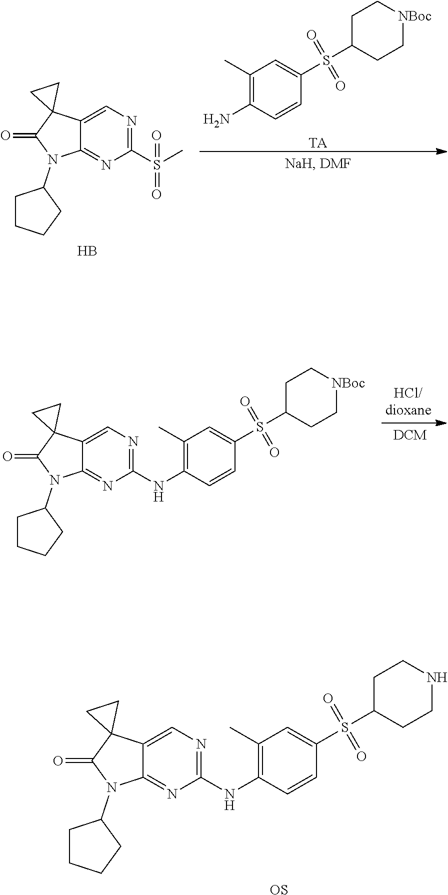

### Step 1—Tert-butyl 4-[4-[(7′-cyclopentyl-6′-oxo-spiro[cyclopropane-1,5′-pyrrolo[2,3-d]pyrimidine]-2′-yl)amino]-3-methyl-phenyl]sulfonylpiperidine-1-carboxylate

A solution of 7′-cyclopentyl-2′-methylsulfonyl-spiro[cyclopropane-1,5′-pyrrolo[2,3-d]pyrimidine]-6′-one (50 mg, 163 umol, Intermediate HB), tert-butyl 4-(4-amino-3-methyl-phenyl)sulfonylpiperidine-1-carboxylate (57.66 mg, 163 umol, Intermediate TA), and NaH (26.0 mg, 651 umol, 60% dispersion in mineral oil) in DMF (1 mL) was stirred at 25° C. for 1 hr. On completion, the reaction mixture was quenched by addition of sat. NH₄Cl at 25° C., and then diluted with H₂O and extracted with EA. The combined organic layers were washed with brine, dried over Na₂SO₄, filtered and concentrated in vacuo to give a residue. Then the residue was purified by prep-HPLC (column: Phenomenex luna C18 150*25 mm*10 um; mobile phase: [water(FA)-ACN]; B %: 47%-77%, 8 min) to give the title compound (30 mg, 15.9% yield) as a white solid. ¹H NMR (400 MHz, DMSO-d₆) δ 8.94 (s, 1H), 8.06-7.91 (m, 2H), 7.65 (d, J=1.6 Hz, 1H), 7.62-7.56 (m, 1H), 4.81-4.64 (m, 1H), 4.10-3.93 (m, 2H), 3.44-3.39 (m, 1H), 2.80-2.64 (m, 2H), 2.37 (s, 3H), 2.13-2.01 (m, 2H), 1.90-1.79 (m, 4H), 1.78-1.68 (m, 4H), 1.57-1.48 (m, 4H), 1.36 (s, 9H), 1.34-1.26 (m, 2H). LC-MS (ESI⁺) m/z 582.3 (M+1)+.

### Step 2—7′-Cyclopentyl-2′-[2-methyl-4-(4-piperidylsulfonyl)anilino]spiro[cyclopropane-1,5′-pyrrolo[2,3-d]pyrimidine]-6′-one

To a solution of tert-butyl 4-[4-[(7′-cyclopentyl-6′-oxo-spiro[cyclopropane-1,5′-pyrrolo [2,3-d]pyrimidine]-2′-yl)amino]-3-methyl-phenyl]sulfonylpiperidine-1-carboxylate (25 mg, 42.9 umol) in DCM (1 mL) was added HCl/dioxane (4 M, 10.7 uL). The mixture was then stirred at 25° C. for 1 hr. On completion, the reaction mixture was filtered and concentrated in vacuo to give the title compound (20 mg, 97% yield). LC-MS (ESI⁺) m/z 482.0 (M+H)⁺.

### Synthesis of 2-[4-[4-[(7′-Cyclopentyl-6′-oxo-spiro[cyclopropane-1,5′-pyrrolo[2,3-d]pyrimidine]-2′-yl) amino]-3-methyl-phenyl]sulfonyl-1-piperidyl]ethyl methanesulfonate (Intermediate OT)

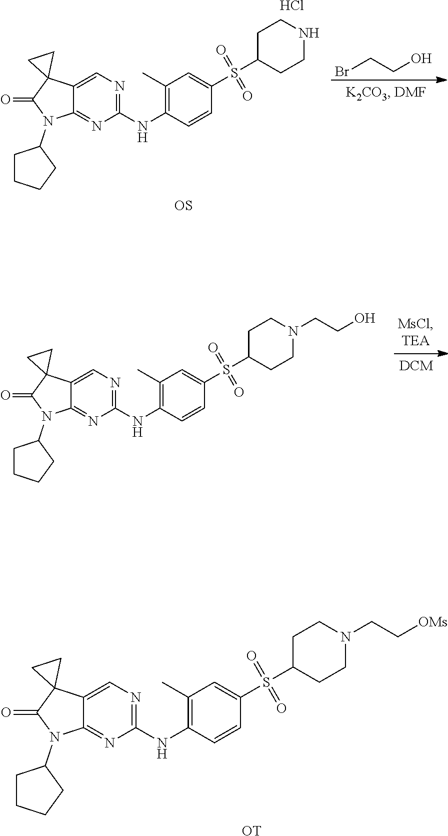

### Step 1—7′-Cyclopentyl-2′-[4-[[1-(2-hydroxyethyl)-4-piperidyl]sulfonyl]-2-methyl-anilino]spiro [cyclopropane-1,5′-pyrrolo[2,3-d]pyrimidine]-6′-one

To a solution of 7′-cyclopentyl-2′-[2-methyl-4-(4-piperidylsulfonyl)anilino]spiro[cyclopropane-1,5′-pyrrolo[2,3-d]pyrimidine]-6′-one (240 mg, 463 umol, HCl, Intermediate OS) in DMF (2 mL) was added K₂CO₃ (128 mg, 926 umol). Then 2-bromoethanol (32.9 uL, 463 umol) was added and the mixture was stirred at 25° C. for 10 hrs. On completion, the mixture was filtered and the filtrate was extracted with EA (30 mL×3). The combined organic layers were concentrated in vacuo to give the residue. The residue was purified by prep-HPLC (column: Waters xbridge 150*25 mm 10 um; mobile phase: [water (NH₄HCO₃)-ACN]; B %: 30%-60%, 11 min) to give the title compound (120 mg, 49% yield) as white solid. ¹H NMR (400 MHz, DMSO-d₆) δ 8.92 (s, 1H), 7.99-7.94 (m, 2H), 7.65 (d, J=1.6 Hz, 1H), 7.59-7.57 (m, 1H), 4.74-4.69 (m, 1H), 4.35 (t, J=5.2 Hz, 1H), 3.44-3.40 (m, 2H), 3.14-3.08 (m, 1H), 2.91 (d, J=11.2 Hz, 2H), 2.36 (s, 3H), 2.33 (t, J=6.4 Hz, 2H), 2.09-2.04 (m, 2H), 1.95-1.90 (m, 2H), 1.81-1.78 (m, 4H), 1.75-1.66 (m, 4H), 1.56-1.44 (m, 6H). LC-MS (ESI⁺) m/z 526.1 (M+H)⁺.

### Step 2—2-[4-[4-[(7′-Cyclopentyl-6′-oxo-spiro[cyclopropane-1,5′-pyrrolo[2,3-d]pyrimidine]-2′-yl)amino]-3-methyl-phenyl]sulfonyl-1-piperidyl]ethyl methanesulfonate

To a solution of 7′-cyclopentyl-2′-[4-[[1-(2-hydroxyethyl)-4-piperidyl]sulfonyl]-2-methyl-anilino]spiro[cyclopropane-1,5′-pyrrolo[2,3-d]pyrimidine]-6′-one (120 mg, 228 umol) in DCM (1 mL) was added TEA (127 uL, 913 umol). Then methane sulfonyl chloride (243 uL, 3.14 mmol) was added at 0° C., and the mixture was stirred at 25° C. for 1 hr. On completion, the mixture was poured into ice water (30 mL) slowly, then extracted with DCM (20 mL×3). The combined organic layers were dried over anhydrous Na₂SO₄ and concentrated in vacuo to give the title compound (110 mg, 79% yield) as yellow oil. LC-MS (ESI⁺) m z 604.1 (M+H)⁺.

### Synthesis of 1-[(4-methoxyphenyl)methyl]-3-(3-methyl-2-oxo-4-piperazin-1-yl-benzimidazol-1-yl) piperidine-2,6-dione (Intermediate OU)

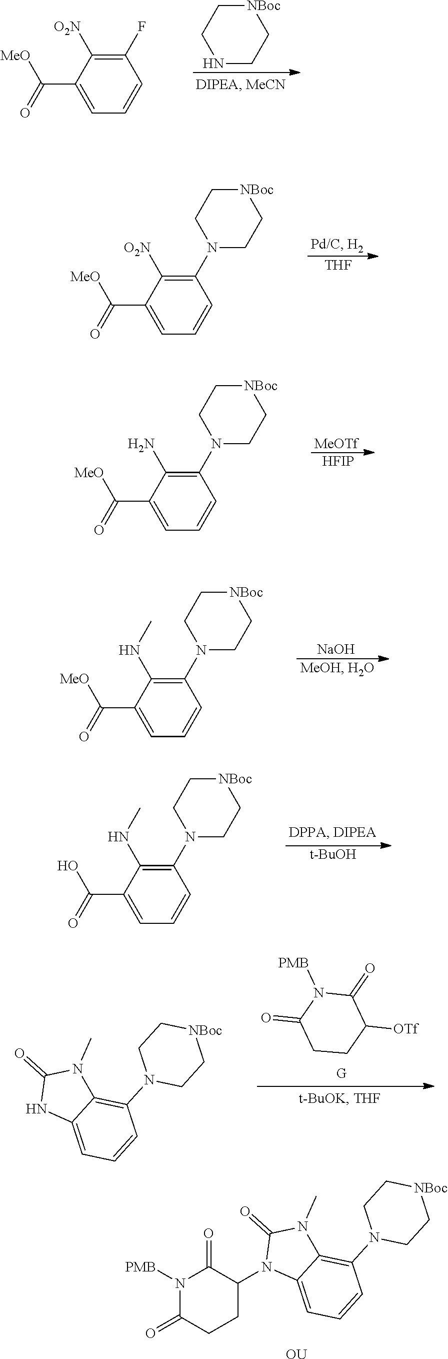

### Step 1—Tert-butyl 4-(3-methoxycarbonyl-2-nitro-phenyl)piperazine-1-carboxylate

To a solution of methyl 3-fluoro-2-nitro-benzoate (10.0 g, 50.2 mmol, CAS #1214353-57-7) and tert-butyl piperazine-1-carboxylate (11.2 g, 60.3 mmol, CAS #143238-38-4) in ACN (100 mL) was added DIPEA (19.5 g, 151 mmol). The reaction mixture was stirred at 50° C. for 12 hrs. On completion, the mixture was concentrated in vacuo. The residue was dissolved in water (200 mL), then extracted with EA (2×200 mL). The organic layer was washed with brine (2×100 mL), dried with Na₂SO₄, filtered and the filtrate was concentrated in vacuo to give the title compound (18.3 g, 100% yield) as yellow oil. ¹H NMR (400 MHz, DMSO-d₆) δ 7.86 (dd, J=1.2, 8.0 Hz, 1H), 7.82-7.79 (m, 1H), 7.74-7.68 (m, 1H), 3.83 (s, 3H), 3.40-3.35 (m, 4H), 2.88-2.84 (m, 4H), 1.41 (s, 9H).

### Step 2—Tert-butyl 4-(2-amino-3-methoxycarbonyl-phenyl)piperazine-1-carboxylate

To a solution of tert-butyl 4-(3-methoxycarbonyl-2-nitro-phenyl)piperazine-1-carboxylate (17.0 g, 46.5 mmol) in THF (15 mL) was added Pd/C (2.00 g, 10 wt %). The reaction mixture was stirred at 20° C. for 12 hrs under H₂ (15 Psi) atmosphere. On completion, the mixture was filtered and the filtrate was concentrated in vacuo to give the title compound (15.2 g, 97% yield) as a yellow solid. ¹H NMR (400 MHz, CDCl₃) δ 7.67 (dd, J=1.2, 8.0 Hz, 1H), 7.10 (dd, J=1.2, 7.6 Hz, 1H), 6.61 (t, J=7.6 Hz, 1H), 6.24 (br s, 2H), 4.28-3.95 (m, 2H), 3.87 (s, 3H), 3.16-2.84 (m, 4H), 2.80-2.55 (m, 2H), 1.49 (s, 9H).

### Step 3—Tert-butyl 4-[3-methoxycarbonyl-2-(methylamino)phenyl]piperazine-1-carboxylate

To a solution of tert-butyl 4-(2-amino-3-methoxycarbonyl-phenyl)piperazine-1-carboxylate (15.0 g, 44.7 mmol) in 1,1,1,3,3,3-hexafluoropropan-2-ol (40 mL) was added methyl trifluoromethanesulfonate (9.54 g, 58.1 mmol) at 0° C. The reaction mixture was stirred at 0° C. for 0.5 hr. On completion, the mixture was diluted with water (200 mL), then extracted with EA (2×200 mL). The organic layer was washed with brine (2×200 mL), dried with Na₂SO₄, filtered and the filtrate was concentrated in vacuo to give the title compound (15.0 g, 96% yield) as a yellow solid. ¹H NMR (400 MHz, DMSO-d₆) δ 8.16-7.90 (m, 1H), 7.46 (dd, J=1.2, 8.0 Hz, 1H), 7.23 (d, J=7.6 Hz, 1H), 6.77 (t, J=7.6 Hz, 1H), 3.80 (s, 3H), 3.55-3.45 (m, 4H), 2.87 (s, 3H), 2.80-2.74 (m, 4H), 1.42 (s, 9H).

### Step 4-3-(4-Tert-butoxycarbonylpiperazin-1-yl)-2-(methylamino)benzoic acid

To a solution of tert-butyl 4-[3-methoxycarbonyl-2-(methylamino)phenyl]piperazine-1-carboxylate (14.0 g, 40.1 mmol) in a mixed solvent of H₂O (20 mL) and MeOH (140 mL) was added NaOH (4.81 g, 120 mmol). The reaction mixture was stirred at 70° C. for 12 hrs. On completion, the mixture was concentrated in vacuo. The residue was diluted with water (200 mL), and extracted with EA (100 mL). The organic layer was discarded. The aqueous phase was acidified with HCl (1N) to pH=3-5, and extracted with EA (2×100 mL). The organic layer was washed with brine (200 mL), dried with Na₂SO₄, filtered and the filtrate was concentrated in vacuo. The residue was triturated with MeOH/H₂O (1:10, 100 mL) and filtered. The filter cake was dried in vacuo to give the title compound (9.60 g, 71% yield) as a white solid. LC-MS (ESI⁺) m/z 336.1 (M+H)⁺.

### Step 5—Tert-butyl 4-(3-methyl-2-oxo-1H-benzimidazol-4-yl)piperazine-1-carboxylate

To a solution of 3-(4-tert-butoxycarbonylpiperazin-1-yl)-2-(methylamino)benzoic acid (9.60 g, 28.6 mmol) and DIPEA (11.1 g, 85.9 mmol) in t-BuOH (200 mL) was added DPPA (7.88 g, 28.6 mmol). The reaction mixture was stirred at 85° C. for 12 hrs. On completion, the mixture was concentrated in vacuo. The residue was diluted with water (200 mL), and extracted with EA (2×200 mL). The organic layer was washed with brine (200 mL) and concentrated in vacuo. The residue was purified by reverse phase (0.1% FA condition) to give the title compound (3.35 g, 35% yield) as a white solid. ¹H NMR (400 MHz, DMSO-d₆) δ 10.84 (s, 1H), 6.94-6.87 (m, 1H), 6.85-6.79 (m, 1H), 6.75 (dd, J=1.2, 7.6 Hz, 1H), 4.06-3.80 (m, 2H), 3.55 (s, 3H), 3.20-2.87 (m, 4H), 2.76-2.56 (m, 2H), 1.42 (s, 9H).

### Step 6—Tert-butyl 4-[1-[1-[(4-methoxyphenyl)methyl]-2,6-dioxo-3-piperidyl]-3-methyl-2-oxo-benzimidazol-4-yl]piperazine-1-carboxylate

To a solution of tert-butyl 4-(3-methyl-2-oxo-1H-benzimidazol-4-yl)piperazine-1-carboxylate (3.30 g, 9.93 mmol) in THF (50 mL) was added t-BuOK (1.67 g, 14.9 mmol) at 0° C. 1 hr later, and a solution of [1-[(4-methoxyphenyl)methyl]-2,6-dioxo-3-piperidyl] trifluoromethanesulfonate (4.54 g, 11.9 mmol, Intermediate G) in THF (20 mL) was added. The reaction mixture was stirred at 0° C. for 3 hrs. On completion, the mixture was acidified with FA to pH=3-5, diluted with water (300 mL), then extracted with EA (2×300 mL). The organic layer was washed with brine (200 mL) and concentrated in vacuo. The residue was purified by reverse phase flash (0.1% FA condition) to give the title compound (3.90 g, 70% yield) as a white solid. LC-MS (ESI⁺) m/z 564.3 (M+H)⁺.

### Synthesis of 2-Methylsulfonyl-8-spiro[2.4]heptan-7-yl-pyrido[2,3-d]pyrimidin-7-one (Intermediate OV)

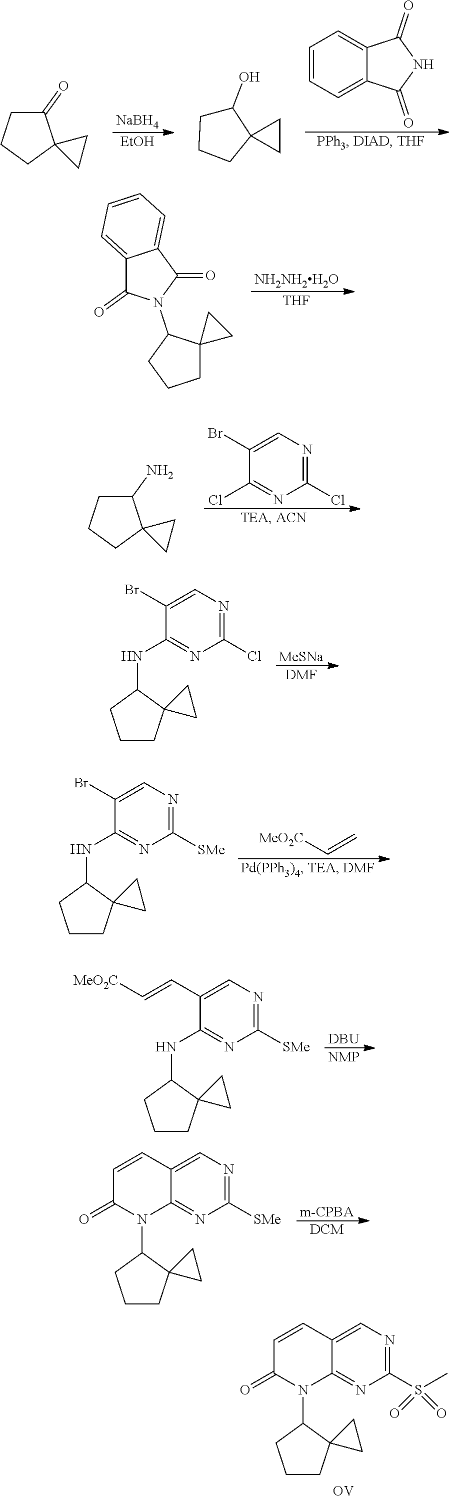

### Step 1—Spiro[2.4]heptan-7-ol

A mixture of spiro[2.4]heptan-7-one (12 g, 108.94 mmol, CAS #5771-32-4) in EtOH (100 mL) was degassed and purged with N₂ for 3 times, then NaBH₄ (6.34 g, 167 mmol) was added to the mixture at 0° C. Then the mixture was stirred at 25° C. for 2 hrs under N₂ atmosphere. On completion, the reaction mixture was quenched by addition of aq. NH₄Cl (20 mL) at 0° C., and then diluted with H₂O (300 mL) and extracted with EA (3×100 mL). The combined organic layers were washed with brine (2×50 mL), dried over anhydrous Na₂SO₄, filtered and concentrated in vacuo to give the title compound (9.40 g, 77% yield) as colorless oil. ¹H NMR (400 MHz, DMSO-d₆) δ 4.23 (d, J=2.8 Hz, 1H), 3.53-3.44 (m, 1H), 1.91-1.82 (m, 1H), 1.80-1.72 (m, 2H), 1.64-1.50 (m, 2H), 1.41-1.33 (m, 1H), 0.76-0.70 (m, 1H), 0.42-0.37 (m, 1H), 0.37-0.26 (m, 2H).

### Step 2-2-Spiro[2.4]heptan-7-ylisoindoline-1,3-dione

To a solution of spiro[2.4]heptan-7-ol (9.40 g, 83.8 mmol), isoindoline-1,3-dione (18.4 g, 125 mmol, CAS #85-41-6) and PPh₃ (32.9 g, 125 mmol) in THF (90 mL) was added DIAD (25.4 g, 125 mmol, 24.4 mL) at 25° C. under N₂. The reaction was then stirred at 60° C. for 16 hrs. On completion, the reaction mixture was diluted with H₂O (200 mL) and extracted with EA (3×100 mL). The combined organic layers were washed with brine (2×50 mL), dried over anhydrous Na₂SO₄, filtered and concentrated in vacuo to give a residue. The residue was purified by column chromatography (SiO₂, Petroleum ether/Ethyl acetate=100/1 to 10/1) to give the title compound (1.80 g, 8% yield) as a white solid. ¹H NMR (400 MHz, DMSO-d₆) δ 7.83 (s, 4H), 4.35-4.29 (m, 1H), 2.25-2.11 (m, 3H), 2.02-1.93 (m, 1H), 1.79-1.66 (m, 1H), 1.44-1.39 (m, 1H), 0.61-0.53 (m, 1H), 0.50-0.40 (m, 2H), 0.31-0.23 (m, 1H). LC-MS (ESI⁺) m/z 242.0 (M+H)⁺.

### Step 3—Spiro[2.4]heptan-7-amine

To a solution of 2-spiro[2.4]heptan-7-ylisoindoline-1,3-dione (6.00 g, 24.8 mmol) in THF (50 mL) was added NH₂NH₂·H₂O (7.47 g, 149 mmol, 7.25 mL, CAS #7803-57-8). The reaction was then stirred at 60° C. for 3 hrs. On completion, the reaction mixture was diluted with THF (30 mL) and filtered. The filtrate was concentrated in vacuo to give the title compound (2.7 g, 98% yield) as colorless oil.

### Step 4—5-Bromo-2-chloro-N-spiro[2.4]heptan-7-yl-pyrimidin-4-amine

To a solution of 5-bromo-2,4-dichloro-pyrimidine (4.61 g, 20.2 mmol, 2.60 mL, CAS #36082-50-5) in ACN (40 mL) was added TEA (2.66 g, 26.3 mmol, 3.80 mL) and spiro[2.4]heptan-7-amine (2.70 g, 24.2 mmol, 2.60 mL). The mixture was then stirred at 25° C. for 6 hrs. On completion, the reaction mixture was diluted with H₂O (200 mL) and extracted with EA (3×100 mL). The combined organic layers were washed with brine (2×50 mL), dried over anhydrous Na₂SO₄, filtered and concentrated in vacuo to give a residue. The residue was purified by column chromatography (SiO₂, Petroleum ether/Ethyl acetate=100/1 to 1/1) to give the title compound (5.2 g, 85% yield) as a white solid. ¹H NMR (400 MHz, CDCl₃) δ 8.08 (s, 1H), 5.47 (d, J=5.6 Hz, 1H), 4.22-4.08 (m, 1H), 2.32-2.23 (m, 1H), 1.92-1.79 (m, 3H), 1.78-1.69 (m, 1H), 1.61-1.54 (m, 1H), 0.73-0.61 (m, 2H), 0.60-0.55 (m, 1H), 0.54-0.48 (m, 1H). LC-MS (ESI⁺) m/z 303.8 (M+H)⁺.

### Step 5—5-Bromo-2-methylsulfanyl-N-spiro[2.4]heptan-7-yl-pyrimidin-4-amine

To a solution of 5-bromo-2-chloro-N-spiro[2.4]heptan-7-yl-pyrimidin-4-amine (3.40 g, 11.2 mmol) in DMF (34 mL) was added sodium methanethiolate (1.97 g, 28.0 mmol, 1.80 mL, CAS #5188-07-8). The mixture was then stirred at 25° C. for 2 hrs. On completion, the reaction mixture was diluted with H₂O (30 mL) and extracted with EA (3×30 mL). The combined organic layers were washed with brine (2×30 mL), dried over anhydrous Na₂SO₄, filtered and concentrated in vacuo to give a residue. The residue was purified by column chromatography (SiO₂, Petroleum ether/Ethyl acetate=100/1 to 10/1) to give the title compound (3.20 g, 91% yield) as a white solid. ¹H NMR (400 MHz, CDCl₃) δ 8.04 (s, 1H), 5.29 (d, J=6.0 Hz, 1H), 4.21-4.12 (m, 1H), 2.47 (s, 3H), 2.31-2.21 (m, 1H), 1.91-1.71 (m, 4H), 1.59-1.51 (m, 1H), 0.71-0.64 (m, 2H), 0.58-0.47 (m, 2H).

### Step 6—Methyl (E)-3-[2-methylsulfanyl-4-(spiro[2.4]heptan-7-ylamino)pyrimidin-5-yl]prop-2-enoate

A mixture of 5-bromo-2-methylsulfanyl-N-spiro[2.4]heptan-7-yl-pyrimidin-4-amine (4.90 g, 15.5 mmol), TEA (4.73 g, 46.7 mmol, 6.50 mL), and Pd(PPh₃)₄ (1.80 g, 1.56 mmol) in DMF (50 mL) was degassed and purged with N₂ three times. Then methyl prop-2-enoate (8.42 g, 97.8 mmol, 8.80 mL) was added the mixture, and then the mixture was stirred at 90° C. for 16 hrs under N₂ atmosphere. On completion, the reaction mixture was diluted with H₂O (50 mL) and extracted with EA (3×30 mL). The combined organic layers were washed with brine (2×20 mL), dried over anhydrous Na₂SO₄, filtered and concentrated in vacuo to give a residue. The residue was purified by column chromatography (SiO₂, Petroleum ether/Ethyl acetate=100/1 to 1/1) to give the title compound (4.2 g, 84% yield) as a yellow solid. ¹H NMR (400 MHz, CDCl₃) δ 8.12 (s, 1H), 7.51 (d, J=16.0 Hz, 1H), 6.27 (d, J=16.0 Hz, 1H), 5.06 (d, J=6.8 Hz, 1H), 4.32-4.16 (m, 1H), 3.81 (s, 3H), 2.55-2.44 (m, 3H), 2.33-2.22 (m, 1H), 1.95-1.87 (m, 1H), 1.85-1.70 (m, 3H), 1.56-1.49 (m, 1H), 0.72-0.61 (m, 2H), 0.59-0.47 (m, 2H).

### Step 7—2-Methylsulfanyl-8-spiro[2.4]heptan-7-yl-pyrido[2,3-d]pyrimidin-7-one

To a solution of methyl (E)-3-[2-methylsulfanyl-4-(spiro[2.4]heptan-7-ylamino)pyrimidin-5-yl]prop-2-enoate (2.50 g, 7.83 mmol) in NMP (2 mL) was added DBU (5.96 g, 39.1 mmol, 5.90 mL) at 25° C. Then the reaction was stirred at 120° C. for 1 hr. On completion, the reaction mixture was diluted with H₂O (30 mL) and extracted with EA (3×20 mL). The combined organic layers were washed with brine (2×10 mL), dried over anhydrous Na₂SO₄, filtered and concentrated in vacuo to give the residue. The residue was purified by reversed-phase HPLC (0.1% FA condition) to give the title compound (2 g, 89% yield) as a yellow solid. ¹H NMR (400 MHz, DMSO-d₆) δ 8.86 (s, 1H), 7.88 (d, J=9.6 Hz, 1H), 6.56 (d, J=9.6 Hz, 1H), 6.02-5.46 (m, 1H), 2.56 (s, 3H), 2.48-2.31 (m, 2H), 2.12-1.95 (m, 2H), 1.85-1.74 (m, 1H), 1.36 (s, 1H), 0.64-0.56 (m, 1H), 0.53-0.33 (m, 2H), 0.03 (d, J=3.2 Hz, 1H). LC-MS (ESI⁺) m/z 288.0 (M+H)⁺.

### Step 8—2-Methylsulfonyl-8-spiro[2.4]heptan-7-yl-pyrido[2,3-d]pyrimidin-7-one

To a solution of 2-methylsulfanyl-8-spiro[2.4]heptan-7-yl-pyrido[2,3-d]pyrimidin-7-one (2.20 g, 7.66 mmol) in DCM (20 mL) was added m-CPBA (4.66 g, 22.9 mmol, 85% solution). The mixture was then stirred at 40° C. for 16 hrs. On completion, the reaction mixture was quenched by addition of Na₂S₂O₃ (5 mL) at 0° C., and then diluted with aq. NaHCO₃ (30 mL) and extracted with DCM (3×15 mL). The combined organic layers were washed with brine (2×10 mL), dried with anhydrous Na₂SO₄, filtered and concentrated in vacuo to give a residue. The residue was purified by column chromatography (SiO₂, Petroleum ether/Ethyl acetate=5/1 to 0/1) to give the title compound (2 g, 6.26 mmol, 82% yield) as a white solid. ¹H NMR (400 MHz, CDCl₃) δ 8.92 (s, 1H), 7.70 (d, J=9.6 Hz, 1H), 6.86 (d, J=9.6 Hz, 1H), 6.04-5.57 (m, 1H), 3.37 (s, 3H), 2.70-2.38 (m, 2H), 2.24-2.11 (m, 2H), 1.96-1.84 (m, 1H), 1.48-1.38 (m, 1H), 0.71-0.60 (m, 2H), 0.55 (d, J=1.6 Hz, 1H), 0.11--0.21 (m, 1H).

### Synthesis of 6-Chloro-2-[2-methyl-4-(4-piperidylsulfonyl)anilino]-8-spiro[2.4]heptan-7-yl-pyrido[2,3-d]pyrimidin-7-one (Intermediate OW)

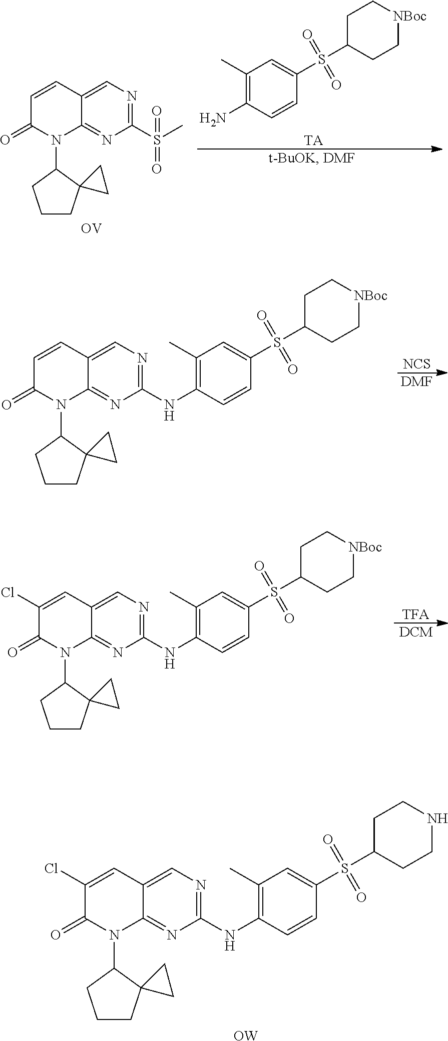

### Step 1—Tert-butyl 4-[3-methyl-4-[(7-oxo-8-spiro[2.4]heptan-7-yl-pyrido[2,3-d]pyrimidin-2-yl)amino]phenyl]sulfonylpiperidine-1-carboxylate

To a solution of tert-butyl 4-(4-amino-3-methyl-phenyl)sulfonylpiperidine-1-carboxylate (332 mg, 939 umol, Intermediate TA) in DMF (3 mL) was added t-BuOK (421 mg, 3.76 mmol) and 2-methylsulfonyl-8-spiro[2.4]heptan-7-yl-pyrido[2,3-d]pyrimidin-7-one (300 mg, 939 umol, Intermediate OV). The mixture was then stirred at 0° C. for 2 hos. On completion, the reaction mixture was quenched by addition H₂O (10 mL) at 25° C., and then extracted with EA (3×10 mL). The combined organic layers were washed with brine (2×10 mL), dried over by anhydrous Na₂SO₄, filtered and concentrated in vacuo to give a residue. Then the residue was purified by prep-HPLC (column: Phenomenex luna C18 150*25 mm*10 um; mobile phase: [water(FA)-ACN]; B %: 50%-80%, 10 min) to give the title compound (317 mg, 56% yield) as a white solid. LC-MS (ESI⁺) m/z 594.4 (M+H)⁺.

### Step 2—Tert-butyl4-[4-[(6-chloro-7-oxo-8-spiro[2.4]heptan-7-yl-pyrido[2,3-d]pyrimidin-2-yl)amino]-3-methyl-phenyl]sulfonylpiperidine-1-carboxylate

To a solution of tert-butyl 4-[3-methyl-4-[(7-oxo-8-spiro[2.4]heptan-7-yl-pyrido[2,3-d]pyrimidin-2-yl)amino]phenyl]sulfonylpiperidine-1-carboxylate (280 mg, 471 umol) in DMF (3 mL) was added NCS (188 mg, 1.41 mmol, CAS #128-09-6). The mixture was then stirred at 70° C. for 1 hr. On completion, the reaction mixture was diluted with water (100 mL) before extracting with EA (3×50 mL). The combined organic phase was washed with brine (100 mL), dried over anhydrous Na₂SO₄, and concentrated in vacuo to give a residue. The residue was purified by prep-TLC (Petroleum ether: Ethyl acetate=5:1) to give the title compound (100 mg, 33% yield) as a white solid. ¹H NMR (400 MHz, DMSO-d₆) δ 9.68 (s, 1H), 8.76 (s, 1H), 8.18 (s, 1H), 7.90-7.78 (m, 1H), 7.73 (s, 1H), 7.66 (d, J=7.6 Hz, 1H), 5.74 (s, 1H), 4.04-3.99 (m, 2H), 3.53-3.39 (m, 2H), 2.83-2.68 (m, 3H), 2.56 (s, 3H), 2.35 (s, 3H), 1.86 (d, J=12.0 Hz, 2H), 1.74-1.59 (m, 1H), 1.37 (s, 9H), 1.34-1.25 (m, 2H), 0.53-0.28 (m, 3H), 0.04 (s, 1H). LC-MS (ESI⁺) m/z 628.2 (M+H)⁺.

### Step 3—6-Chloro-2-[2-methyl-4-(4-piperidylsulfonyl)anilino]-8-spiro[2.4]heptan-7-yl-pyrido[2,3-d]pyrimidin-7-one

To a solution of tert-butyl4-[4-[(6-chloro-7-oxo-8-spiro[2.4]heptan-7-yl-pyrido[2,3-d]pyrimidin-2-yl) amino]-3-methylphenyl]sulfonylpiperidine-1-carboxylate (55.0 mg, 87.5 umol) in DCM (0.5 mL) was added TFA (9.98 mg, 87.5 umol). The mixture was then stirred at 25° C. for 1 hr. On completion, the reaction mixture was diluted with water (100 mL), and extracted with DCM (3×50 mL). The combined organic phase was washed with brine (2×50 mL), dried over anhydrous Na₂SO₄, and concentrated in vacuo to give the title compound (40 mg, 86% yield) as a white solid. LC-MS (ESI⁺) m/z 528.1 (M+H)⁺.

### Synthesis of 2-[1-[1-(2,6-dioxo-3-piperidyl)-3-methyl-2-oxo-benzimidazol-4-yl]-4-piperidyl]acetaldehyde (Intermediate OX)

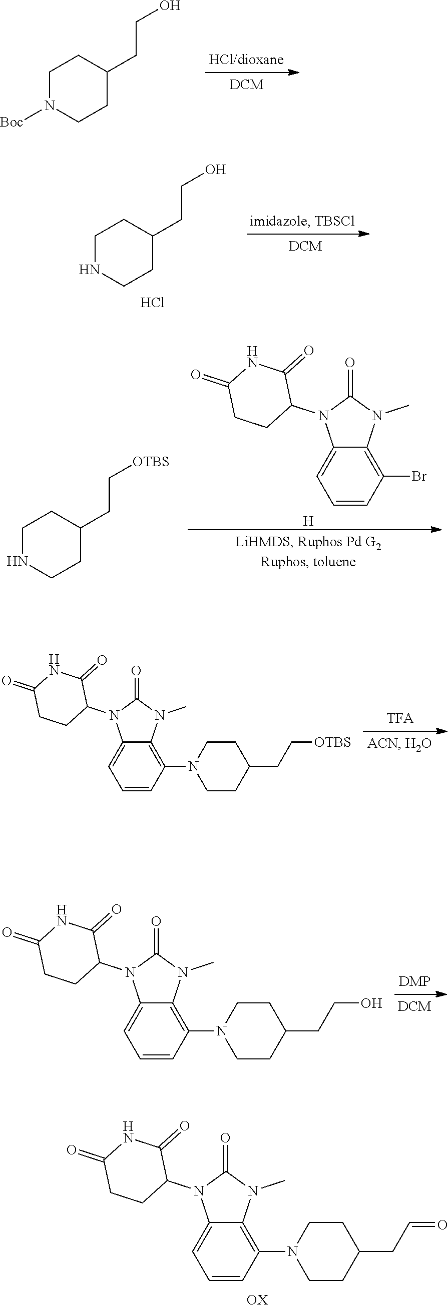

### Step 1—2-(4-Piperidyl)ethanol

To a solution of tert-butyl 4-(2-hydroxyethyl)piperidine-1-carboxylate (5.00 g, 21.8 mmol, CAS#198892-80-7) in DCM (50.0 mL) was added HCl/dioxane (4.00 M, 50.0 mL). The mixture was then stirred at rt for 0.5 hr. On completion, the mixture was concentrated in vacuo. The mixture was diluted with MeOH (50 mL) and stirred with basic ion exchange resin for 1 hr. The mixture was then filtered and the filtrate was concentrated in vacuo to give the title compound (2.8 g, 99% yield) as yellow oil. ¹H NMR (400 MHz, DMSO-d₆) δ 4.49-4.33 (m, 1H), 3.52-3.46 (m, 2H), 3.21-3.16 (m, 2H), 2.85-2.70 (m, 2H), 1.80-1.70 (m, 2H), 1.67-1.54 (m, 1H), 1.51-1.30 (m, 2H), 1.30-1.13 (m, 2H)

### Step 2—Tert-butyl-dimethyl-[2-(4-piperidyl)ethoxy]silane

To a solution of 2-(4-piperidyl) ethanol (2.80 g, 21.6 mmol) in DCM (30.0 mL) was added TBSCl (3.92 g, 26.0 mmol) and imidazole (2.95 g, 43.3 mmol). The mixture was then stirred at rt for 16 hrs. On completion, the mixture was diluted with DCM (50 mL) and washed with H₂O (3×70 mL). The organic layers were washed with brine (3×50 mL) dried over anhydrous Na₂SO₄, filtered and concentrated in vacuo to give the title compound (3 g, 56% yield) as yellow oil. ¹H NMR (400 MHz, CDCl₃) δ 3.66 (t, J=6.4 Hz, 2H), 3.30-3.20 (m, 2H), 2.79-2.62 (m, 2H), 1.83-1.73 (m, 2H), 1.70-1.55 (m, 1H), 1.52-1.45 (m, 2H), 1.43-1.29 (m, 2H), 0.92 (s, 9H), 0.10 (s, 6H)

### Step 3—3-[4-[4-[2-[Tert-butyl(dimethyl)silyl]oxyethyl]-1-piperidyl]-3-methyl-2-oxo-benzimidazol-1-yl]piperidine-2,6-dione

To a solution of tert-butyl-dimethyl-[2-(4-piperidyl)ethoxy]silane (863 mg, 3.55 mmol), 3-(4-bromo-3-methyl-2-oxobenzimidazol-1-yl)piperidine-2,6-dione (600 mg, 1.77 mmol, Intermediate H) in toluene (10.0 mL) was added [2-(2-aminophenyl)phenyl]-chloro-palladium dicyclohexyl-[2-(2,6-diisopropoxyphenyl)phenyl]phosphane (137 mg, 177 umol), RuPhos (82.8 mg, 177 umol) and LiHMDS (1.00 M, 8.87 mL) under N₂. The mixture was then stirred at 80° C. for 1 hr under N₂. On completion, the mixture was concentrated in vacuo. Then diluted with DMF (6.00 mL), filtered and the filtrate was acidified with FA until the pH=5. The filtrate was concentrated in vacuo. The mixture was then purified by reverse phase: (0.1% FA) to give the title compound (460 mg, 51% yield) as yellow solid. ¹H NMR (400 MHz, DMSO-d₆) δ 11.08 (s, 1H), 7.02-6.78 (m, 3H), 5.40-5.30 (m, 1H), 3.67 (t, J=6.4 Hz, 2H), 3.61 (s, 3H), 3.15-3.05 (m, 2H), 2.97-2.81 (m, 1H), 2.74-2.66 (m, 2H), 2.65-2.56 (m, 2H), 2.04-1.93 (m, 1H), 1.80-1.70 (m, 2H), 1.55-1.45 (m, 3H), 1.44-1.31 (m, 2H), 0.88 (s, 9H), 0.05 (s, 6H), LC-MS (ESI⁺) m/z 501.2 (M+H)⁺.

### Step 4—3-[4-[4-(2-Hydroxyethyl)-1-piperidyl]-3-methyl-2-oxo-benzimidazol-1-yl]piperidine-2,6-dione

To a solution of 3-[4-[4-[2-[tert-butyl(dimethyl)silyl]oxyethyl]-1-piperidyl]-3-methyl-2-oxo-benzimidazol-1-yl]piperidine-2,6-dione (400 mg, 798 umol) in a mixture solvent of ACN (4.00 mL) and H₂O (0.5 mL) was added TFA (1.54 g, 13.5 mmol). The mixture was then stirred at 25° C. for 1 hr. On completion, the mixture was concentrated in vacuo. The mixture was diluted with H₂O (10 mL) and extracted with EA (3×10 mL). The organic layer were washed with brine (2×10 mL), dried over anhydrous Na₂SO₄, filtered and concentrated in vacuo to give the title compound (280 mg, 90% yield) as yellow solid. ¹H NMR (400 MHz, DMSO-d₆) δ 11.08 (s, 1H), 7.07-6.78 (m, 3H), 5.40-5.30 (m, 1H), 4.38 (t, J=5.2 Hz, 1H), 3.62 (s, 3H), 3.52-3.44 (m, 2H), 3.15-3.05 (m, 2H), 2.95-2.81 (m, 1H), 2.75-2.58 (m, 4H), 2.04-1.94 (m, 1H), 1.84-1.71 (m, 2H), 1.59-1.26 (m, 5H), LC-MS (ESI⁺) m/z 387.1 (M+H)⁺.

### Step 5—2-[1-[1-(2,6-Dioxo-3-piperidyl)-3-methyl-2-oxo-benzimidazol-4-yl]-4-piperidyl]acetaldehyde

To a solution of 3-[4-[4-(2-hydroxyethyl)-1-piperidyl]-3-methyl-2-oxo-benzimidazol-1-yl]piperidine-2,6-dione (100 mg, 258.7 umol) in DCM (3.00 mL) was added DMP (164 mg, 388 umol) and NaHCO₃ (108 mg, 1.29 mmol). The mixture was then stirred at rt for 1 hr. On completion, the mixture was diluted with DCM (15 mL), quenched with saturated Na₂S₂O₃ (15 mL) and washed with saturated NaHCO₃ (2×15 mL). The organic layer was dried over anhydrous Na₂SO₄, filtered and concentrated in vacuo to give the title compound (95 mg, 95% yield) as yellow solid. ¹H NMR (400 MHz, DMSO-d₆) δ 11.08 (s, 1H), 9.75-9.65 (m, 1H), 7.02-6.78 (m, 3H), 5.38-5.28 (m, 1H), 3.61 (s, 3H), 3.15-3.05 (m, 2H), 2.94-2.80 (m, 1H), 2.77-2.64 (m, 3H), 2.64-2.58 (m, 1H), 2.46-2.40 (m 2H), 2.06-1.89 (m, 2H), 1.79-1.72 (m, 2H), 1.50-1.35 (m, 2H), LC-MS (ESI⁺) m/z 385.1 (M+H)⁺

### Synthesis of 2′-[4-(7-Azaspiro[3.5]nonan-2-ylsulfonyl)-2-methyl-anilino]-7′-cyclopentyl-spiro[cyclopropane-1,5′-pyrrolo[2,3-d]pyrimidine]-6′-one (Intermediate OY)

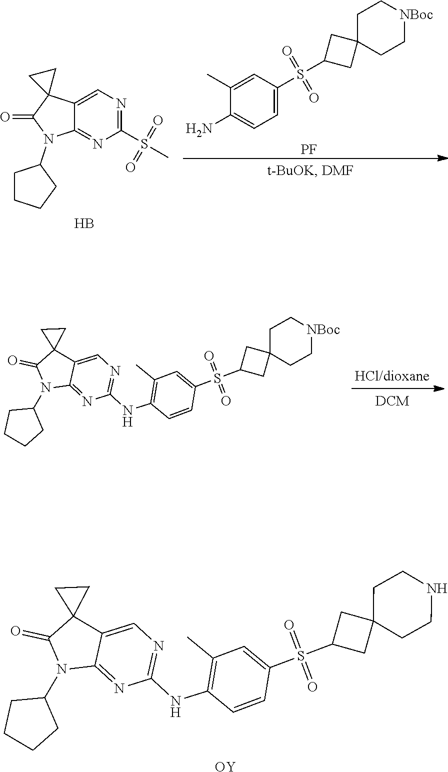

### Step 1—Tert-butyl 2-[4-[(7′-cyclopentyl-6′-oxo-spiro[cyclopropane-1,5′-pyrrolo[2,3-d]pyrimidine]-2′-yl)amino]-3-methyl-phenyl]sulfonyl-7-azaspiro[3.5]nonane-7-carboxylate

To a mixture of tert-butyl 2-(4-amino-3-methyl-phenyl)sulfonyl-7-azaspiro[3.5]nonane-7-carboxylate (400 mg, 1.01 mmol, Intermediate PF) in DMF (8 mL) was added 4A molecular sieves (100 mg), then the t-BuOK (455 mg, 4.06 mmol) was added at 0° C. and the mixture was stirred for 0.1 h. Next, 7′-cyclopentyl-2′-methylsulfonyl-spiro[cyclopropane-1,5′-pyrrolo[2,3-d]pyrimidine]-6′-one (374 mg, 1.22 mmol, Intermediate HB) was added at 0° C., the reaction mixture was stirred at 0° C. for 1 hr. On completion, the reaction mixture was filtered and the filtrate was concentrated in vacuo. The residue was purified by prep-HPLC to give the title compound (150 mg, 24% yield) as black brown solid. ¹H NMR (400 MHz, DMSO-d₆) δ 8.89 (s, 1H), 8.01-7.91 (m, 2H), 7.70-7.56 (m, 2H), 4.72 (quin, J=8.4 Hz, 1H), 4.08 (quin, J=8.4 Hz, 1H), 3.40 (s, 1H), 2.35 (s, 3H), 2.14-2.03 (m, 6H), 1.99-1.92 (m, 2H), 1.86-1.72 (m, 5H), 1.71-1.66 (m, 2H), 1.55-1.51 (m, 3H), 1.50-1.40 (m, 5H), 1.37 (s, 9H). LC-MS (ESI⁺) m/z 622.6 (M+H)⁺.

### Step 2—2′-[4-(7-Azaspiro[3.5]nonan-2-ylsulfonyl)-2-methyl-anilino]-7′-cyclopentyl-spiro[cyclopropane-1,5′-pyrrolo[2,3-d]pyrimidine]-6′-one

To a mixture of tert-butyl 2-[4-[(7′-cyclopentyl-6′-oxo-spiro[cyclopropane-1,5′-pyrrolo[2,3-d]pyrimidine]-2′-yl)amino]-3-methyl-phenyl]sulfonyl-7-azaspiro[3.5]nonane-7-carboxylate (100 mg, 161 umol) in DCM (1 mL) was added HCl/dioxane (1 M, 161 uL), then the mixture was stirred at 25° C. for 0.5 hr. On completion, the reaction mixture was concentrated in vacuo to give the title compound (85.0 mg, 95% yield, HCl) as black brown solid. LC-MS (ESI⁺) m/z 522.1 (M+H)⁺.

### Synthesis of 4-[4-[[Tert-butyl(diphenyl)silyl]oxymethyl]cyclohexyl]sulfonyl-2-methyl-aniline (Intermediate OZ)

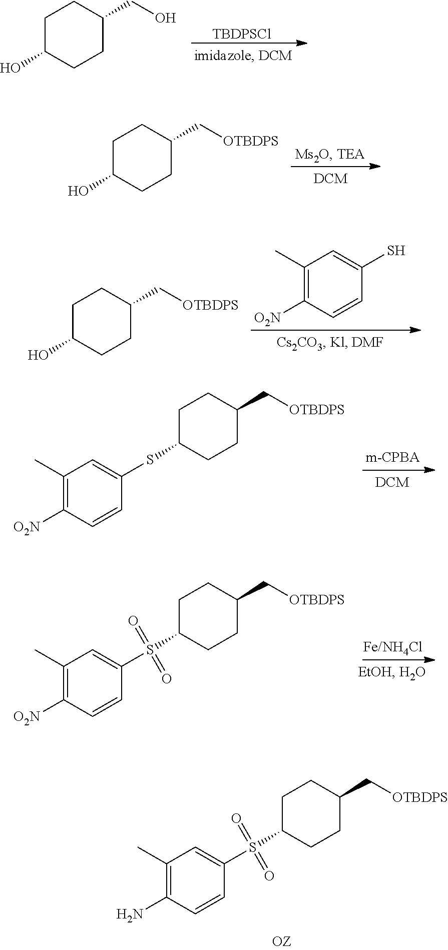

### Step 1—4-[[Tert-butyl(diphenyl)silyl]oxymethyl]cyclohexanol

To a solution of 4-(hydroxymethyl)cyclohexanol (8.00 g, 61.4 mmol, CAS #3685-24-3) in DMF (80 mL) was added TBDPS-Cl (17.7 g, 64.5 mmol, 16.5 mL) and imidazole (5.02 g, 73.7 mmol). The mixture was then stirred at 25° C. for 14 hrs. On completion, the mixture was diluted with H₂O (100 mL), and extracted with EA (2×30 mL). The organic layers were washed with brine (2×30 mL), dried over anhydrous Na₂SO₄, filtered and concentrated in vacuo to give a residue. The residue was purified by prep-HPLC (column: Phenomenex luna C18 250*50 mm*10 um; mobile phase: [water(FA)-ACN]; B %: 65%-95%, 20 min) to give the title compound (3.5 g, 16% yield) as colorless oil. ¹H NMR (400 MHz, CDCl₃) δ 7.71-7.65 (m, 4H), 7.45-7.37 (m, 6H), 4.00 (d, J=2.4 Hz, 1H), 3.53 (d, J=6.4 Hz, 2H), 1.95 (s, 1H), 1.76-1.68 (m, 2H), 1.67-1.56 (m, 5H), 1.48-1.38 (m, 2H), 1.07 (s, 9H).

### Step 2 [4-[[Tert-butyl(diphenyl)silyl]oxymethyl]cyclohexyl]methanesulfonate

To a solution of 4-[[tert-butyl(diphenyl)silyl]oxymethyl]cyclohexanol (3.20 g, 8.68 mmol) in DCM (30 mL) was added TEA (1.76 g, 17.3 mmol, 2.5 mL) and methylsulfonyl methanesulfonate (2.27 g, 13.0 mmol). The mixture was then stirred at 25° C. for 2 hrs. On completion, the mixture was diluted with H₂O (100 mL), and extracted with EA (2×30 mL). The organic layers were washed with brine (2×30 mL), dried over anhydrous Na₂SO₄, filtered and concentrated in vacuo to give the title compound (3.80 g, 98% yield) as colorless oil. ¹H NMR (400 MHz, CDCl₃) δ 7.68-7.64 (m, 4H), 7.46-7.37 (m, 6H), 5.00 (s, 1H), 3.51 (d, J=6.0 Hz, 2H), 3.01 (s, 3H), 2.10-2.05 (m, 2H), 1.66-1.61 (m, 4H), 1.48-1.39 (m, 3H), 1.06 (s, 9H).

### Step 3—Tert-butyl-[[4-(3-methyl-4-nitro-phenyl)sulfanylcyclohexyl]methoxy]-diphenyl-silane

To a solution of [4-[[tert-butyl(diphenyl)silyl]oxymethyl]cyclohexyl] methanesulfonate (4.10 g, 9.18 mmol) and 3-methyl-4-nitro-benzenethiol (1.63 g, 9.64 mmol, CAS #53827-87-5) in DMF (40 mL) was added Cs₂CO₃ (7.48 g, 22.9 mmol) and KI (304 mg, 1.84 mmol). The mixture was then stirred at 70° C. for 14 hrs. On completion, the mixture was diluted with H₂O (100 mL), and extracted with EA (2×30 mL). The organic layers were washed with brine (2×30 mL), dried over by anhydrous Na₂SO₄, filtered and concentrated in vacuo to give a residue. The residue was purified by column chromatography (SiO₂, PE/EA=100/1 to 98/1) to give the title compound (1.80 g, 38% yield) as yellow oil. ¹H NMR (400 MHz, CDCl₃) δ 8.01-7.91 (m, 1H), 7.68-7.64 (m, 4H), 7.45-7.37 (m, 6H), 7.24-7.18 (m, 2H), 3.50 (d, J=6.0 Hz, 2H), 3.25-3.14 (m, 1H), 2.62-2.59 (m, 3H), 2.18-2.10 (m, 2H), 1.96-1.88 (m, 2H), 1.65-1.57 (m, 1H), 1.43-1.37 (m, 2H), 1.22-1.10 (m, 2H), 1.07-1.05 (m, 9H).

### Step 4—Tert-butyl-[[4-(3-methyl-4-nitro-phenyl)sulfonylcyclohexyl]methoxy]-diphenyl-silane

To a solution of tert-butyl-[[4-(3-methyl-4-nitro-phenyl)sulfanylcyclohexyl]methoxy]-diphenyl-silane (1.80 g, 3.40 mmol) in DCM (20 mL) was added m-CPBA (3.52 g, 17.3 mmol, 85% solution) at 0° C. The mixture was then stirred at 40° C. for 2 hrs. On completion, the reaction mixture was quenched by addition of Na₂S₂O₃ (3 mL) at 0° C., and then diluted with aq. NaHCO₃ (30 mL) and extracted with DCM (3×15 mL). The combined organic layers were washed with brine (2×10 mL), dried with anhydrous Na₂SO₄, filtered and concentrated in vacuo to give a residue. The residue was purified by column chromatography (SiO₂, PE/EA=100/1 to 20/1) to give the title compound (1.91 g, 100% yield) as yellow oil.

### Step 5—4-[4-[[Tert-butyl(diphenyl)silyl]oxymethyl]cyclohexyl]sulfonyl-2-methyl-aniline

To a solution of tert-butyl-[[4-(3-methyl-4-nitro-phenyl)sulfonylcyclohexyl]methoxy]-diphenyl-silane (1.91 g, 3.46 mmol) in EtOH (20 mL) and H₂O (2 mL) was added Fe (966 mg, 17.3 mmol) and NH₄Cl (925 mg, 17.3 mmol). The mixture was then stirred at 80° C. for 1 hr. On completion, the reaction mixture was concentrated in vacuo to remove EtOH. The residue was purified by column chromatography (SiO₂, Petroleum ether/Ethyl acetate=100/1 to 98/1) to give the title compound (1.4 g, 74% yield) as a yellow oil. ¹H NMR (400 MHz, DMSO-d₆) δ 7.57 (dd, J=1.6, 7.6 Hz, 4H), 7.46-7.38 (m, 6H), 7.33-7.27 (m, 2H), 6.71-6.65 (m, 1H), 5.90-5.84 (m, 2H), 3.40 (d, J=6.0 Hz, 2H), 2.92-2.83 (m, 1H), 2.11-2.05 (m, 3H), 1.91 (d, J=11.2 Hz, 2H), 1.79 (d, J=11.2 Hz, 2H), 1.72-1.54 (m, 3H), 1.43-1.36 (m, 2H), 0.97 (s, 9H). LC-MS (ESI⁺) m/z 522.0 (M+H)⁺.

### Synthesis of 4-[4-[(7′-Cyclopentyl-6′-oxo-spiro[cyclopropane-1,5′-pyrrolo[2,3-d]pyrimidine]-2′-yl)amino]-3-methyl-phenyl]sulfonylcyclohexanecarbaldehyde (Intermediate PA)

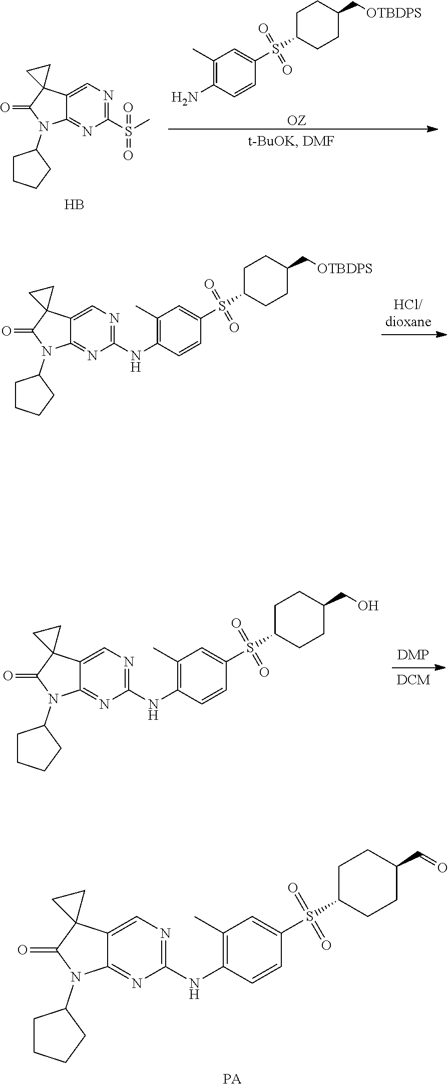

### Step 1—2′-[4-[4-[[Tert-butyl(diphenyl)silyl]oxymethyl]cyclohexyl]sulfonyl-2-methyl-anilino]-7′-cyclopentyl-spiro[cyclopropane-1,5′-pyrrolo [2,3-d]pyrimidine]-6′-one

To a solution of 4-[4-[[tert-butyl(diphenyl)silyl]oxymethyl]cyclohexyl]sulfonyl-2-methyl-aniline (200 mg, 383 umol, Intermediate OZ) in DMF (2 mL) was added t-BuOK (172 mg, 1.53 mmol) and 7′-cyclopentyl-2′-methylsulfonyl-spiro[cyclopropane-1,5′-pyrrolo[2,3-d]pyrimidine]-6′-one (129 mg, 421 umol, Intermediate HB). The mixture was then stirred at 25° C. for 1 hr. On completion, the reaction mixture was diluted with H₂O (10 mL) and extracted with EA (10 mL×3). The combined organic layers were washed with brine (5 mL×2), dried over anhydrous Na₂SO₄, filtered and concentrated in vacuo to give a residue. The residue was purified by prep-TLC (SiO₂, PE:EA=4:1) to give the title compound (140 mg, 49% yield) as a white solid. ¹H NMR (400 MHz, CDCl₃) δ 8.48 (d, J=8.4 Hz, 1H), 7.74-7.68 (m, 3H), 7.66-7.60 (m, 4H), 7.44-7.35 (m, 6H), 4.89-4.80 (m, 1H), 3.45 (d, J=6.0 Hz, 2H), 2.92-2.77 (m, 2H), 2.48-2.42 (m, 3H), 2.24 (dd, J=9.2, 12.0 Hz, 2H), 2.15 (d, J=11.2 Hz, 2H), 1.98-1.89 (m, 6H), 1.82-1.79 (m, 2H), 1.73-1.64 (m, 3H), 1.61-1.57 (m, 2H), 1.54-1.43 (m, 3H), 1.05-1.02 (m, 9H), 1.01-0.92 (m, 1H). LC-MS (ESI⁺) m/z 749.8 (M+H)⁺.

### Step 2 7′-Cyclopentyl-2′-[4-[4-(hydroxymethyl)cyclohexyl]sulfonyl-2-methyl-anilino]spiro[cyclopropane-1,5′-pyrrolo[2,3-d]pyrimidine]-6′-one

A solution of 2′-[4-[4-[[tert-butyl(diphenyl)silyl]oxymethyl]cyclohexyl]sulfonyl-2-methyl-anilino]-7′-cyclopentyl-spiro[cyclopropane-1,5′-pyrrolo[2,3-d]pyrimidine]-6′-one (130 mg, 173 umol) in HCl/dioxane (1 mL, 4M) was stirred at 25° C. for 14 hrs. On completion, the reaction mixture was concentrated under reduced pressure to give a residue. The residue was purified by prep-TLC (SiO₂, PE:EA=0:1) to give the title compound (86.0 mg, 92% yield) as a white solid. LC-MS (ESI⁺) m/z 511.4 (M+H)⁺.

### Step 3—4-[4-[(7′-Cyclopentyl-6′-oxo-spiro[cyclopropane-1,5′-pyrrolo[2,3-d]pyrimidine]-2′-yl)amino]-3-methyl-phenyl]sulfonylcyclohexanecarbaldehyde

To a solution of 7′-cyclopentyl-2′-[4-[4-(hydroxymethyl)cyclohexyl]sulfonyl-2-methyl-anilino]spiro [cyclopropane-1,5′-pyrrolo[2,3-d]pyrimidine]-6′-one (86.0 mg, 168 umol) in DCM (1 mL) was added DMP (107 mg, 252 umol). The mixture was then stirred at 25° C. for 2 hos. On completion, the reaction mixture was quenched by addition of Na₂S₂O₃ (0.5 mL) at 25° C., and then diluted with aq. NaHCO₃ (5 mL) and extracted with DCM (5 mL×3). The combined organic layers were washed with brine (3 mL×2), dried over Na₂SO₄, filtered and concentrated in vacuo to give the title compound (85 mg, 99% yield) as yellow oil. LC-MS (ESI⁺) m/z 509.3 (M+H)⁺.

### Synthesis of 2-[4-(7-Azaspiro[3.5]nonan-2-ylsulfonyl)-2-methyl-anilino]-6-chloro-8-spiro[2.4]heptan-7-yl-pyrido[2,3-d]pyrimidin-7-one (Intermediate PB)

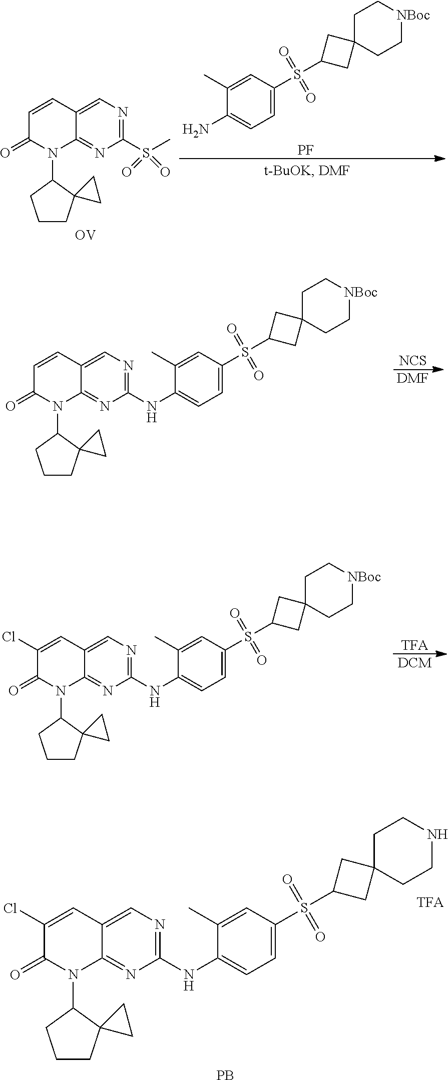

### Step 1—Tert-butyl 2-[3-methyl-4-[(7-oxo-8-spiro[2.4]heptan-7-yl-pyrido[2,3-d]pyrimidin-2-yl)amino]phenyl]sulfonyl-7-azaspiro[3.5]nonane-7-carboxylate

To a solution of tert-butyl 2-(4-amino-3-methyl-phenyl)sulfonyl-7-azaspiro[3.5]nonane-7-carboxylate (30.0 mg, 76.0 umol, Intermediate PF) in DMF (2 mL) was added t-BuOK (34.1 mg, 304 umol) and 2-methylsulfonyl-8-spiro[2.4]heptan-7-yl-pyrido[2,3-d]pyrimidin-7-one (24.2 mg, 76.0 umol, Intermediate OV). The mixture was then stirred at 0° C. for 1 hr. On completion, the reaction mixture was partitioned between H₂O (10 mL) and EA (10 mL). The organic phase was separated, washed with EA (10 mL×2), dried over anhydrous sodium sulfate, filtered and concentrated in vacuo to give a residue. The residue was purified by prep-TLC (SiO2, PE:EA=1:1) to give the title compound (30.0 mg, 62% yield) as a white solid. LCMS (ESI⁺) m/z 634.1 (M+H)⁺.

### Step 2—Tert-butyl 2-[4-[(6-chloro-7-oxo-8-spiro[2.4]heptan-7-yl-pyrido[2,3-d]pyrimidin-2-yl) amino]-3-methyl-phenyl]sulfonyl-7-azaspiro[3.5]nonane-7-carboxylate

To a solution of tert-butyl 2-[3-methyl-4-[(7-oxo-8-spiro[2.4]heptan-7-yl-pyrido[2,3-d]pyrimidin-2-yl) amino]phenyl]sulfonyl-7-azaspiro[3.5]nonane-7-carboxylate (30.0 mg, 47.3 umol) in DMF (1 mL) was added NCS (18.9 mg, 142 umol). The mixture was then stirred at 70° C. for 1 hr. On completion, the reaction mixture was concentrated in vacuo to give a residue. The residue was purified by prep-HPLC (column: Phenomenex luna C18 150*25 mm*10 um; mobile phase: [water(FA)-ACN]; B %: 62%-92%, 9 min) to give the title compound (18.0 mg, 56% yield) as a white solid. ¹H NMR (400 MHz, DMSO-d₆) δ 9.67 (s, 1H), 8.75 (s, 1H), 8.23-8.10 (m, 1H), 7.84-7.64 (m, 3H), 5.79-5.52 (m, 1H), 4.25-4.03 (m, 1H), 3.25-3.17 (m, 4H), 2.34 (s, 3H), 2.14-2.07 (m, 2H), 2.03-1.91 (m, 4H), 1.75-1.61 (m, 1H), 1.58-1.47 (m, 3H), 1.43 (s, 2H), 1.37 (s, 9H), 1.32-1.09 (m, 2H), 0.58-0.30 (m, 3H), 0.15-0.01 (m, 1H). LCMS (ESI⁺) m/z 668.2 (M+H)⁺.

### Step 3—2-[4-(7-Azaspiro[3.5]nonan-2-ylsulfonyl)-2-methyl-anilino]-6-chloro-8-spiro[2.4]heptan-7-yl-pyrido[2,3-d]pyrimidin-7-one

To a solution of tert-butyl 2-[4-[(6-chloro-7-oxo-8-spiro[2.4]heptan-7-yl-pyrido[2,3-d]pyrimidin-2-yl) amino]-3-methyl-phenyl]sulfonyl-7-azaspiro[3.5]nonane-7-carboxylate (18.0 mg, 26.9 umol) in DCM (0.6 mL) was added TFA (0.2 mL). The mixture was then stirred at 25° C. for 0.5 hr. On completion, the reaction mixture was concentrated in vacuo to give the title compound (18.0 mg, 97% yield, TFA) as a white solid. LCMS (ESI⁺) m/z 568.2 (M+H)⁺.

### Synthesis of 1-[3-(2,6-Dioxo-3-piperidyl)-2-oxo-1,3-benzoxazol-7-yl]piperidine-4-carbaldehyde (Intermediate PC)

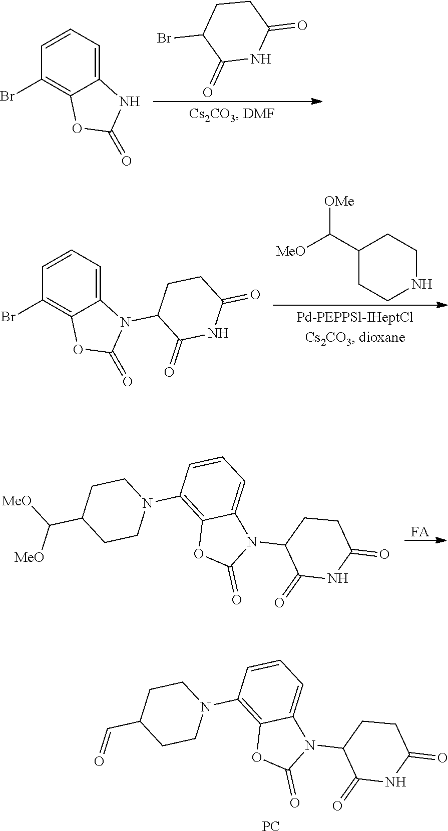

### Step 1—3-(7-Bromo-2-oxo-1,3-benzoxazol-3-yl)piperidine-2,6-dione

To a solution of 7-bromo-3H-1,3-benzoxazol-2-one (2.00 g, 9.35 mmol, CAS #871367-14-5) and 3-bromopiperidine-2,6-dione (3.59 g, 18.6 mmol) in DMF (20 mL) was added Cs₂CO₃ (6.09 g, 18.6 mmol) at 0° C., then the mixture was stirred at 50° C. for 16 hrs. On completion, the mixture was filtered, diluted with water (150 mL) and extracted with EA (3×100 mL). The combined organic layer was dried over anhydrous Na₂SO₄, filtered and concentrated in vacuo to give the residue. The residue was purified by column chromatography (SiO₂, PE:EA=5:1 to 1:1) to give the compound (1.00 g, 33% yield) as yellow solid. ¹H NMR (400 MHz, DMSO-d₆) δ ppm 2.59-2.76 (m, 3H) 2.82-2.93 (m, 1H) 5.39 (m, 1H) 7.15-7.21 (m, 1H) 7.28-7.32 (m, 1H) 7.38 (d, J=8.2 Hz, 1H) 11.24 (s, 1H). LC-MS (ESI⁺) m/z 326.8 (M+H)⁺.

### Step 2—3-[7-[4-(Dimethoxymethyl)-1-piperidyl]-2-oxo-1,3-benzoxazol-3-yl]piperidine-2,6-dione

To a solution of 3-(7-bromo-2-oxo-1,3-benzoxazol-3-yl)piperidine-2,6-dione (500 mg, 1.54 mmol) and 4-(dimethoxymethyl)piperidine (269 mg, 1.69 mmol, CAS #188646-83-5) in dioxane (5 mL) was added Cs₂CO₃ (1.00 g, 3.08 mmol) and 1,3-bis[2,6-bis(1-propylbutyl)phenyl]-4,5-dichloro-2H-imidazol-1-ium-2-ide 3-chloropyridine dichloropalladium (150 mg, 154 umol). Then the mixture was stirred at 100° C. for 16 hrs. On completion, the mixture was filtered, diluted with EA (3×100 mL) and extracted with water (250 mL). The combined organic layer was dried over anhydrous Na₂SO₄, filtered and concentrated in vacuo to give the residue. The residue was purified by column chromatography (SiO₂, PE:EA=10:1 to 1:5) to give the compound (90.0 mg, 15% yield) as brown solid. LC-MS (ESI⁺) m/z 404.2 (M+H)⁺.

### Step 3—1-[3-(2,6-Dioxo-3-piperidyl)-2-oxo-1,3-benzoxazol-7-yl]piperidine-4-carbaldehyde

To a solution of 3-[7-[4-(dimethoxymethyl)-1-piperidyl]-2-oxo-1, 3-benzoxazol-3-yl] piperidine-2,6-dione (30.0 mg, 74.3 umol) was added HCOOH (0.5 mL), then the mixture was stirred at 80° C. for 2 hours. On completion, the mixture was concentrated in vacuo to give the title compound (26.0 mg, 97% yield) as a brown solid. LC-MS (ESI⁺) m/z 358.1 (M+H)⁺.

### Synthesis of 1-Cyclopentyl-3-methyl-7-methylsulfonyl-4H-pyrimido[4,5-d]pyrimidin-2-one (Intermediate PD)

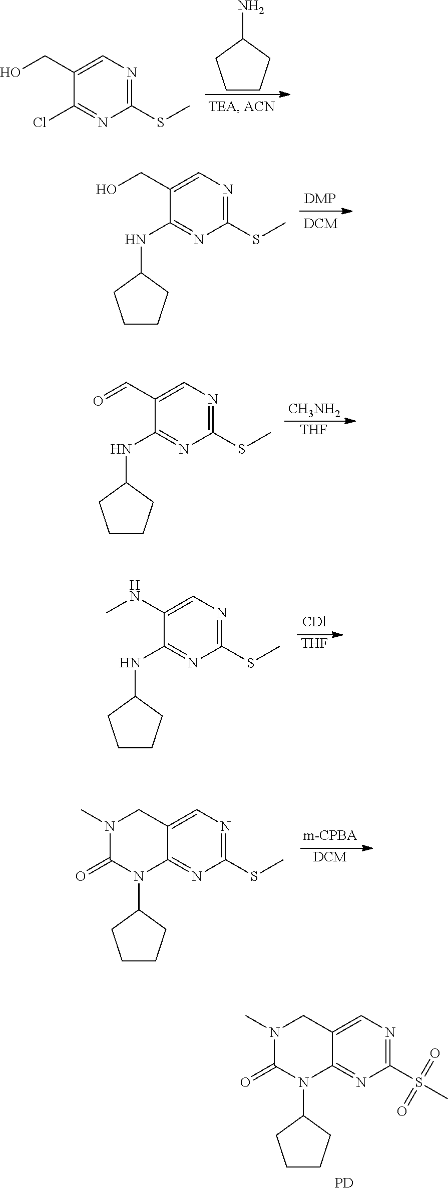

### Step 1—[4-(Cyclopentylamino)-2-methylsulfanyl-pyrimidin-5-yl]methanol

To a solution of (4-chloro-2-methylsulfanyl-pyrimidin-5-yl)methanol (2.00 g, 10.4 mmol, CAS#1044145-59-6) in ACN (20 mL) was added TEA (2.65 g, 26.2 mmol) and cyclopentanamine (937 mg, 11.0 mmol, CAS #1003-03-8) dropwise at 25° C. Then the reaction mixture was stirred at 80° C. for 2 hrs. On completion, the reaction mixture was quenched with H₂O (5 mL) under stirring. The residue was diluted with water (50 mL) and extracted with EA (50 mL×3). The combined organic layer was dried over Na₂SO₄, filtered and concentrated in vacuo to give the title compound (2.00 g, 79% yield) as a light yellow solid. LC-MS (ESI⁺) m/z 240.1 (M+H)⁺.

### Step 2—4-(Cyclopentylamino)-2-methylsulfanyl-pyrimidine-5-carbaldehyde

To a solution of [4-(cyclopentylamino)-2-methylsulfanyl-pyrimidin-5-yl]methanol (1.50 g, 6.27 mmol) in DCM (60 mL) was added DMP (3.19 g, 7.52 mmol) at 0° C., then the reaction mixture was stirred at 25° C. for 1 hr. On completion, the reaction mixture was quenched with sat. Na₂S₂O₃ solution (30 mL) and NaHCO₃ solution (30 mL) under stirring. The residue was diluted with water (50 mL) and extracted with EA (50 mL×3). The combined organic layer was dried over Na₂SO₄, filtered and concentrated in vacuo to give a residue. The residue was purified by flash silica gel chromatography (SiO₂, Petroleum ether/Ethyl acetate=1/1, P1: Rf=0.7) to give the title compound (0.90 g, 60% yield) as a light yellow oil. ¹H NMR (400 MHz, DMSO-d₆) δ=9.74 (s, 1H), 8.57 (d, J=7.2 Hz, 1H), 4.44 (t, J=6.8 Hz, 1H), 2.50-2.49 (m, 4H), 2.07-1.98 (m, 2H), 1.74-1.65 (m, 2H), 1.65-1.56 (m, 2H), 1.56-1.46 (m, 2H); LC-MS (ESI⁺) m/z 238. (M+H)⁺.

### Step 3—N4-cyclopentyl-N5-methyl-2-methylsulfanyl-pyrimidine-4,5-diamine

To a solution of methanamine hydrochloride (1.19 g, 17.7 mmol) in THF (20 mL) was added TEA (2.09 g, 20.6 mmol) at 25° C., then the mixture was stirred at 25° C. for 10 mins. Next, 4-(cyclopentylamino)-2-methylsulfanyl-pyrimidine-5-carbaldehyde (700 mg, 2.95 mmol) was added to the mixture. Next, AcOH (177 mg, 2.95 mmol) was added and the mixture was stirred at 25° C. for 0.5 hour. Keeping the reaction liquid at 25° C., NaBH(OAc)₃ (812 mg, 3.83 mmol) was added slowly. Then the reaction was stirred for 32 hours at 25° C. On completion, the reaction mixture was quenched with water (5 mL) and concentrated in vacuo to give a residue. The residue was diluted with water (5 mL) and extracted with EA (15 mL×3). The combined organic layer was dried over Na₂SO₄, filtered and concentrated in vacuo to give the title compound (160 mg, 39% yield) as a brown solid. LC-MS (ESI⁺) m/z 249.1 (M+H)⁺.

### Step 4—1-Cyclopentyl-3-methyl-7-methylsulfanyl-4H-pyrimido[4,5-d]pyrimidin-2-one

To a solution of N4-cyclopentyl-N5-methyl-2-methylsulfanyl-pyrimidine-4,5-diamine (270 mg, 1.13 mmol) in ACN (10 mL) was added CDI (551 mg, 3.40 mmol) and TEA (229 mg, 2.27 mmol) at 25° C. Then the reaction mixture was stirred at 85° C. for 30 hrs. On completion, the reaction mixture was quenched with water (4 mL), diluted with water (10 mL) and extracted with EA (10 mL×3). The combined organic layer was dried over Na₂SO₄, filtered and concentrated in vacuo to give a residue. The residue was purified by flash silica gel chromatography (SiO₂, Petroleum ether/Ethyl acetate=2/1, P1: Rf=0.5) to give the title compound (150 mg, 47% yield) as a light yellow oil. 1H NMR (400 MHz, DMSO-d₆) δ=8.22 (s, 1H), 5.16 (q, J=8.8 Hz, 1H), 4.33 (s, 2H), 2.91 (s, 3H), 2.48 (s, 3H), 2.13-2.05 (m, 2H), 1.92-1.84 (m, 2H), 1.79-1.71 (m, 2H), 1.59-1.51 (m, 2H), LC-MS (ESI⁺) m/z 279.1 (M+H)⁺.

### Step 5—1-Cyclopentyl-3-methyl-7-methylsulfonyl-4H-pyrimido[4,5-d]pyrimidin-2-one

To a solution of 1-cyclopentyl-3-methyl-7-methylsulfanyl-4H-pyrimido[4,5-d]pyrimidin-2-one (140 mg, 502 umol) in DCM (1 mL) was added m-CPBA (433 mg, 1.51 mmol) dropwise at 0° C., then the reaction mixture was stirred at 25° C. for 2 hrs. On completion, the reaction mixture was quenched with sat. Na₂S₂O₃ solution (3 mL) and NaHCO₃ solution (3 mL) under stirring. The mixture was then filtered, and diluted with water (5 mL). The filtrate was extracted with EA (3×5 mL), the combined organic layer was dried with anhydrous Na₂SO₄, filtered and concentrated in vacuo to give the title compound (140 mg, 89% yield) as a light yellow solid. LC-MS (ESI⁺) m/z 311.1 (M+H)⁺.

### Synthesis of 7-[4-(7-Azaspiro[3.5]nonan-2-ylsulfonyl)-2-methyl-anilino]-1-cyclopentyl-3-methyl-4H-pyrimido[4,5-d]pyrimidin-2-one (Intermediate PE)

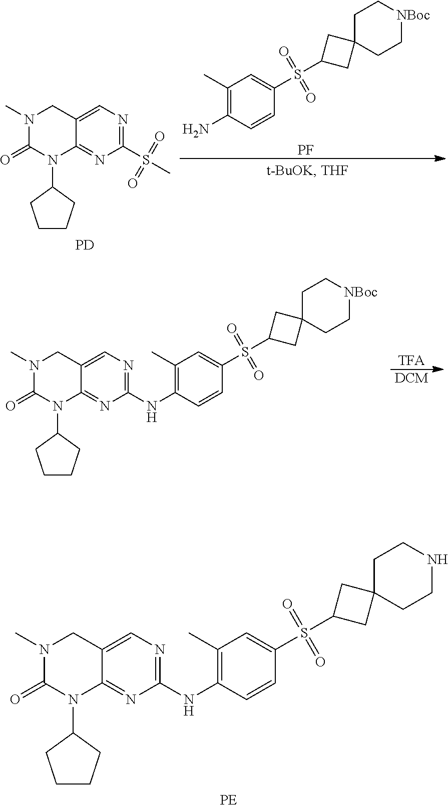

### Step 1—Tert-butyl2-[4-[(1-cyclopentyl-3-methyl-2-oxo-4H-pyrimido[4,5-d]pyrimidin-7-yl)amino]-3-methyl-phenyl]sulfonyl-7-azaspiro[3.5]nonane-7-carboxylate

To a solution of tert-butyl 2-(4-amino-3-methyl-phenyl)sulfonyl-7-azaspiro[3.5]nonane-7-carboxylate (90.8 mg, 230 umol, Intermediate PF) in DMF (2 mL) was added t-BuOK (70.5 mg, 628 umol) dropwise at 0° C. Then, 1-cyclopentyl-3-methyl-7-methylsulfonyl-4H-pyrimido[4,5-d]pyrimidin-2-one (65.0 mg, 209 umol, Intermediate PD) was added and the reaction mixture was stirred at 0° C. for 1 hr. On completion, the reaction mixture was quenched with H₂O (1 mL) under stirring. The residue was diluted with water (2 mL) and extracted with EA (3 mL×3). The combined organic layer was dried over Na₂SO₄, filtered and concentrated in vacuo to give a residue. The residue was purified by reverse phase (0.1% FA condition) to give the title compound (64.0 mg, 48% yield) as a light yellow solid. LC-MS (ESI⁺) m/z 625.2 (M+H)⁺.

### Step 2—7-[4-(7-Azaspiro[3.5]nonan-2-ylsulfonyl)-2-methyl-anilino]-1-cyclopentyl-3-methyl-4H-pyrimido[4,5-d]pyrimidin-2-one

To a solution of tert-butyl 2-[4-[(1-cyclopentyl-3-methyl-2-oxo-4H-pyrimido[4,5-d]pyrimidin-7-yl)amino]-3-methyl-phenyl]sulfonyl-7-azaspiro[3.5]nonane-7-carboxylate (60.0 mg, 96.0 umol) in DCM (1.5 mL) was added TFA (462 mg, 4.05 mmol) dropwise at 25° C., then the reaction mixture was stirred at 25° C. for 1 hr. On completion, the reaction mixture was concentrated in vacuo to give the title compound (50.0 mg, 99% yield) as a light yellow solid. LC-MS (ESI⁺) m/z 525.1 (M+H)⁺.

### Synthesis of Tert-butyl 2-(4-amino-3-methyl-phenyl)sulfonyl-7-azaspiro[3.5]nonane-7-carboxylate (Intermediate PF)

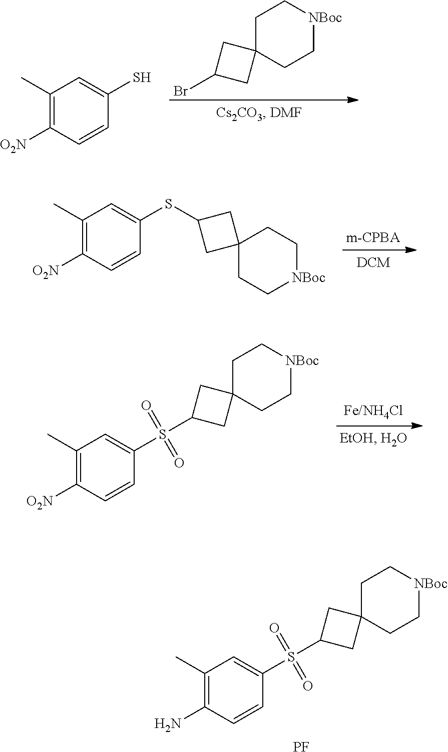

### Step 1—Tert-butyl 2-(3-methyl-4-nitro-phenyl)sulfanyl-7-azaspiro[3.5]nonane-7-carboxylate

A mixture of 3-methyl-4-nitro-benzenethiol (0.600 g, 3.55 mmol, CAS #53827-87-5), tert-butyl 2-bromo-7-azaspiro[3.5]nonane-7-carboxylate (1.08 g, 3.55 mmol, CAS #1225276-07-2) and Cs₂CO₃ (1.27 g, 3.90 mmol) in DMF (30 mL) was stirred at 70° C. for 19 hrs under N₂. The mixture was added to water (60 mL) and extracted with ethyl acetate (3×30 mL). The combined organic phase was dried over anhydrous sodium sulfate, filtered and concentrated to give a residue. The residue was purified by prep-TLC (SiO₂, Petroleum ether/Ethyl acetate=5/1) to give the title compound (650 mg, 47% yield) as a yellow solid. ¹H NMR (400 MHz, CDCl₃) δ 7.96 (d, J=9.2 Hz, 1H), 7.07-6.99 (m, 2H), 4.03-3.87 (m, 1H), 3.42-3.27 (m, 4H), 2.61 (s, 3H), 2.54-2.44 (m, 2H), 1.97-1.88 (m, 2H), 1.67-1.61 (m, 4H), 1.46 (s, 9H).

### Step 2—Tert-butyl 2-(3-methyl-4-nitro-phenyl)sulfonyl-7-azaspiro[3.5]nonane-7-carboxylate

To a mixture of tert-butyl 2-(3-methyl-4-nitro-phenyl)sulfanyl-7-azaspiro[3.5]nonane-7-carboxylate (650 mg, 1.66 mmol) in DCM (20 mL) was added m-CPBA (1.01 g, 4.97 mmol, 85% solution), then the mixture was stirred at 25° C. for 1 hr. On completion, the reaction mixture was quenched by addition of Na₂SO₃ (70 mL), and extracted with DCM (3×30 mL). The separated organic layer was washed with an aqueous Na₂CO₃ (3×30 mL) and dried over anhydrous Na₂SO₄, filtered and the filtrate was concentrated under reduced pressure to give a residue. The residue was purified by prep-TLC (SiO₂, Petroleum ether/Ethyl acetate=1/1) to give the title compound (510 mg, 73% yield) as a white solid. ¹H NMR (400 MHz, CDCl₃) δ 8.06 (d, J=8.4 Hz, 1H), 7.91-7.81 (m, 2H), 3.87-3.71 (m, 1H), 3.38-3.27 (m, 4H), 2.67 (s, 3H), 2.41-2.32 (m, 2H), 2.11-2.03 (m, 2H), 1.66-1.61 (m, 2H), 1.59-1.57 (m, 1H), 1.56-1.53 (m, 1H), 1.45 (s, 9H).

### Step 3—Tert-butyl 2-(4-amino-3-methyl-phenyl)sulfonyl-7-azaspiro[3.5]nonane-7-carboxylate

To a mixture of tert-butyl 2-(3-methyl-4-nitro-phenyl)sulfonyl-7-azaspiro[3.5]nonane-7-carboxylate (510 mg, 1.20 mmol) in EtOH (18 mL) and H₂O (18 mL) was added Fe (335 mg, 6.01 mmol) and NH₄Cl (643 mg, 12.0 mmol), then the mixture was stirred at 80° C. for 2 hrs. On completion, the mixture was filtered to give a clear liquid. Then water (20 mL) was added and the mixture was extracted with ethyl acetate (3×25 mL), The combined organic phase was dried over anhydrous sodium sulfate, filtered and concentrated to give the title compound (510 mg) as a yellow solid. ¹H NMR (400 MHz, DMSO-d₆) δ 7.37-7.27 (m, 2H), 6.68 (d, J=8.4 Hz, 1H), 5.86 (s, 2H), 3.95-3.78 (m, 1H), 3.25-3.11 (m, 4H), 2.08 (s, 3H), 2.05-1.99 (m, 2H), 1.95-1.87 (m, 2H), 1.50-1.42 (m, 2H), 1.41-1.38 (m, 2H), 1.37 (s, 9H).

### Synthesis of 2-[4-(7-Azaspiro[3.5]nonan-2-ylsulfonyl)-2-methyl-anilino]-6-chloro-8-cyclopentyl-pyrido[2,3-d]pyrimidin-7-one (Intermediate PG)

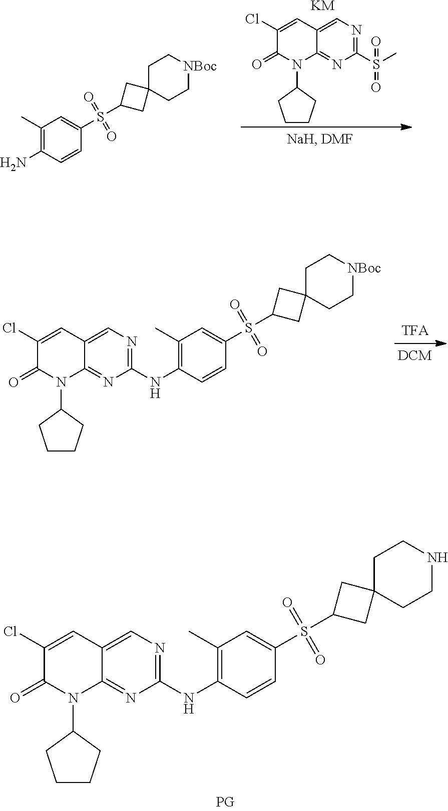

### Step 1—Tert-butyl 2-[4-[(6-chloro-8-cyclopentyl-7-oxo-pyrido[2,3-d]pyrimidin-2-yl)amino]-3-methyl-phenyl]sulfonyl-7-azaspiro[3.5]nonane-7-carboxylate

To a mixture of tert-butyl 2-(4-amino-3-methyl-phenyl)sulfonyl-7-azaspiro[3.5]nonane-7-carboxylate (170 mg, 431 umol, Intermediate PF), 6-chloro-8-cyclopentyl-2-methylsulfonyl-pyrido[2,3-d]pyrimidin-7-one (212 mg, 646 umol, Intermediate KM) and NaH (68.9 mg, 1.72 mmol, 60% dispersion in mineral oil) in DMF (8 mL), then the mixture was stirred at 25° C. for 1 hr under N₂. On completion, the reaction mixture was quenched by addition of H₂O (30 mL) at 25° C., and then extracted with EA (3×10 mL). The combined organic layers were washed with brine (3×10 mL), dried with anhydrous Na₂SO₄, filtered and the filtrate was concentrated in vacuo to give a residue. The residue was purified by prep-HPLC (column: Phenomenex luna C18 150*25 mm*10 um; mobile phase: [water(FA)-ACN]; B %: 61%-91%, 10 min) to give the title compound (80.0 mg, 14% yield) as a yellow solid. ¹H NMR (400 MHz, DMSO-d₆) δ 9.71 (s, 1H), 8.77 (s, 1H), 8.19 (s, 1H), 7.80 (d, J=8.4 Hz, 1H), 7.76-7.64 (m, 2H), 5.83-5.65 (m, 1H), 4.21-4.03 (m, 1H), 3.25-3.16 (m, 4H), 2.35 (s, 3H), 2.17-2.05 (m, 4H), 2.01-1.92 (m, 2H), 1.80-1.62 (m, 4H), 1.55-1.41 (m, 6H), 1.37 (s, 9H). LC-MS (ESI⁺) m/z 642.1 (M+H)⁺.

### Step 2—2-[4-(7-Azaspiro[3.5]nonan-2-ylsulfonyl)-2-methyl-anilino]-6-chloro-8-cyclopentyl-pyrido[2,3-d]pyrimidin-7-one

A mixture of tert-butyl 2-[4-[(6-chloro-8-cyclopentyl-7-oxo-pyrido[2,3-d]pyrimidin-2-yl)amino]-3-methyl-phenyl]sulfonyl-7-azaspiro[3.5]nonane-7-carboxylate (40.0 mg, 62.3 umol) in TFA (0.3 mL) and DCM (1.5 mL) was stirred at 25° C. for 0.5 hr. On completion, the mixture was concentrated in vacuo to give a residue to give the title compound (40.0 mg, 98% yield, TFA) as a yellow oil. LC-MS (ESI⁺) m z 541.9 (M+H)⁺.

### Synthesis of 3-(2-Chloro-4-piperazin-1-yl-phenyl)piperidine-2,6-dione (Intermediate PH)

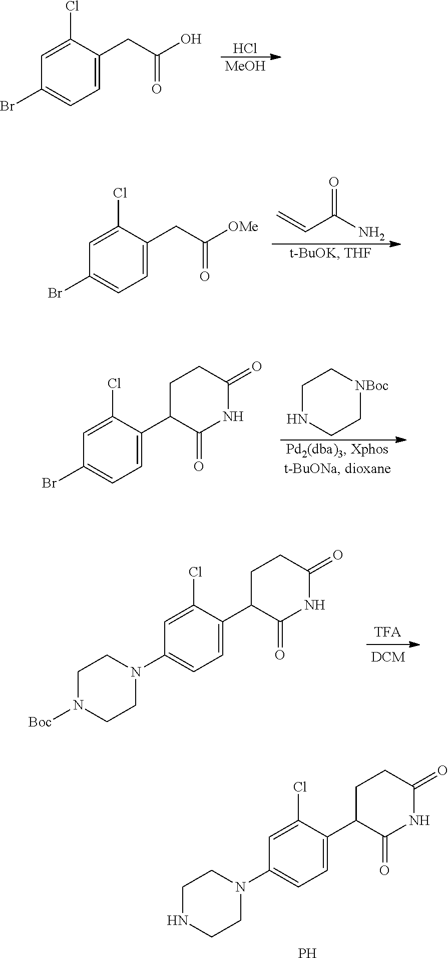

### Step 1—Methyl 2-(4-bromo-2-chloro-phenyl)acetate

To a solution of 2-(4-bromo-2-chloro-phenyl)acetic acid (40.0 g, 160 mmol, CAS #916516-89-7) in MeOH (150 mL) was added HCl (12 M, 13.36 mL) at 0° C. Then the mixture was stirred at 90° C. for 4 hrs under N₂ atmosphere. On completion, the mixture was concentrated in vacuo to remove the MeOH, then diluted with water (40 mL) and saturated NaHCO₃ (100 mL), and extracted with EA (150 mL×3). The combined organic layer was dried over anhydrous Na₂SO₄, filtered and concentrated in vacuo to give the title compound (41.0 g, 97% yield) as yellow oil. ¹H NMR (400 MHz, DMSO-d₆) δ 7.72 (d, J=1.6 Hz, 1H), 7.53 (dd, J=1.6, 8.4 Hz, 1H), 7.38 (d, J=8.0 Hz, 1H), 3.81 (s, 2H), 3.63 (s, 3H).

### Step 2—3-(4-Bromo-2-chloro-phenyl)piperidine-2,6-dione

To a solution of methyl 2-(4-bromo-2-chloro-phenyl)acetate (40.0 g, 151 mmol) and prop-2-enamide (10.7 g, 151 mmol, CAS #9003-05-8) in THF (400 mL) was added t-BuOK (18.7 g, 166 mmol) at 0° C., then the mixture was stirred at 50° C. for 3 hrs under N₂ atmosphere. On completion, the mixture was diluted with saturated NH₄Cl (1 L) and extracted with EA (500 mL×3). The combined organic layer was dried over anhydrous Na₂SO₄, filtered and concentrated in vacuo to give the residue. The residue was purified by triturated with EA:MTBE=1:2 (500 mL) at 25° C. for 1 hr, filtered and the filter cake was dried in vacuo to give the title compound (42.0 g, 91% yield) as white solid. ¹H NMR (400 MHz, DMSO-d₆) δ 10.93 (s, 1H), 7.74 (d, J=2.0 Hz, 1H), 7.55 (dd, J=2.0, 8.4 Hz, 1H), 7.32 (d, J=8.4 Hz, 1H), 4.22 (dd, J=5.2, 12.4 Hz, 1H), 2.81-273 (m, 1H), 2.59-2.52 (m, 1H), 2.37-2.22 (m, 1H), 2.00-1.94 (m, 1H). LC-MS (ESI⁺) m/z 303.9 (M+H)⁺.

### Step 3—Tert-butyl 4-[3-chloro-4-(2,6-dioxo-3-piperidyl)phenyl]piperazine-1-carboxylate

In two parallel reactions, a solution of 3-(4-bromo-2-chloro-phenyl)piperidine-2,6-dione (15.0 g, 49.5 mmol) and tert-butyl piperazine-1-carboxylate (13.8 g, 74.3 mmol, CAS #143238-38-4) in dioxane (250 mL) was added XPhos (2.36 g, 4.96 mmol), Pd₂(dba)₃ (4.54 g, 4.96 mmol) and t-BuONa (14.2 g, 148 mmol). Then the mixture was stirred at 100° C. for 2 hours under N₂ atmosphere. On completion, two parallel reactions of mixture were filtered, the filtrate was acidified with FA (5 mL) until pH=5, diluted with water (600 mL) and extracted with EA (500 mL×3). The combined organic layer was dried over anhydrous Na₂SO₄, filtered and concentrated in vacuo to give the residue. The residue was purified by column chromatography (SiO₂, PE:EA=1:1 to 1:3) to give the title compound (16.0 g, 39% yield) as red solid. ¹H NMR (400 MHz, DMSO-d₆) δ 10.84 (s, 1H), 7.14 (d, J=8.8 Hz, 1H), 6.97 (d, J=2.4 Hz, 1H), 6.91 (dd, J=2.4, 8.8 Hz, 1H), 4.06 (dd, J=5.2, 12.0 Hz, 1H), 3.47-3.40 (m, 4H), 3.15-3.10 (m, 4H), 2.78-2.66 (m, 1H), 2.49-2.47 (m, 1H), 2.28-2.17 (m, 1H), 1.98-1.88 (m, 1H), 1.42 (s, 9H). LC-MS (ESI⁺) m/z 408.1 (M+H)⁺.

### Step 4—3-(2-Chloro-4-piperazin-1-yl-phenyl)piperidine-2,6-dione

To a solution of tert-butyl 4-[3-chloro-4-(2,6-dioxo-3-piperidyl)phenyl]piperazine-1-carboxylate (6.00 g, 14.7 mmol) in DCM (60 mL) was added TFA (23.0 g, 201 mmol), then the mixture was stirred at 25° C. for 1.5 hrs. On completion, the mixture was concentrated in vacuo to give the title compound (6.00 g, 96% yield, TFA) as black brown oil. LC-MS (ESI⁺) m/z 307.9 (M+H)⁺.

### Synthesis of 3-[4-[4-[(4-Aminocyclohexyl)methyl]piperazin-1-yl]-2-chloro-phenyl]piperidine-2,6-dione (Intermediate PI)

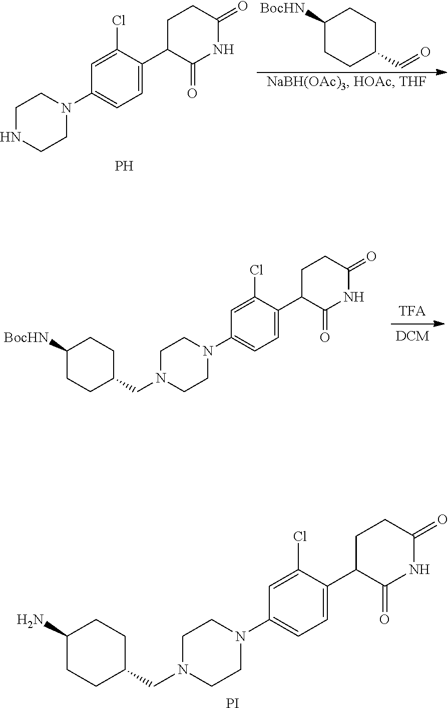

### Step 1—Tert-butyl N-[4-[[4-[3-chloro-4-(2,6-dioxo-3-piperidyl)phenyl]piperazin-1-yl]methyl]cyclohexyl]carbamate

To a solution of 3-(2-chloro-4-piperazin-1-yl-phenyl)piperidine-2,6-dione (6.00 g, 14.2 mmol, TFA, Intermediate PH) in THF (60 mL) was added TEA (4.32 g, 42.6 mmol). Then tert-butyl N-(4-formylcyclohexyl)carbamate (3.56 g, 15.6 mmol, CAS #181308-57-6) was added followed by AcOH (854 mg, 14.2 mmol) was added until the pH=4 at −10° C. The mixture was stirred at −10° C. for 0.5 hr. Then NaBH(OAc)₃ (3.62 g, 17.0 mmol) was added and the mixture was stirred at −10° C. for 0.5 hr. On completion, the mixture was quenched with H₂O (5 mL) at −10° C., diluted with water (80 mL) and extracted with DCM (80 mL×3). The combined organic layer was dried over anhydrous Na₂SO₄, filtered and concentrated in vacuo to give the residue. The residue was purified by reverse-phase (0.1% FA condition) to give the title compound (4.20 g, 56% yield) as white solid. ¹H NMR (400 MHz, DMSO-d₆) δ 10.86 (s, 1H), 7.19 (d, J=8.8 Hz, 1H), 7.06 (s, 1H), 6.99-6.93 (m, 1H), 6.80 (d, J=8.0 Hz, 1H), 4.08 (dd, J=4.8, 12.4 Hz, 1H), 3.86 (d, J=9.6 Hz, 2H), 3.57-3.55 (m, 2H), 3.18-3.00 (m, 6H), 2.80-2.69 (m, 1H), 2.53 (s, 2H), 2.30-2.15 (m, 1H), 1.97-1.87 (m, 1H), 1.84-1.65 (m, 5H), 1.37 (s, 9H), 1.25-1.09 (m, 2H), 1.08-0.94 (m, 2H). LC-MS (ESI⁺) m/z 519.5 (M+H)⁺.

### Step 2—3-[4-[4-[(4-Aminocyclohexyl)methyl]piperazin-1-yl]-2-chloro-phenyl]piperidine-2,6-dione

To a solution of tert-butyl N-[4-[[4-[3-chloro-4-(2,6-dioxo-3-piperidyl)phenyl]piperazin-1-yl]methyl] cyclohexyl]carbamate (4.20 g, 8.09 mmol) in DCM (30 mL) was added TFA (21.4 g, 188 mmol), then the mixture was stirred at 25° C. for 1 hr. On completion, the mixture was concentrated in vacuo to give the title compound (4.10 g, 95% yield, TFA) as brown oil. LC-MS (ESI⁺) m/z 418.9 (M+H)⁺.

### Synthesis of 3-[2-Fluoro-4-(4-piperidyl)anilino]piperidine-2,6-dione (Intermediate PJ)

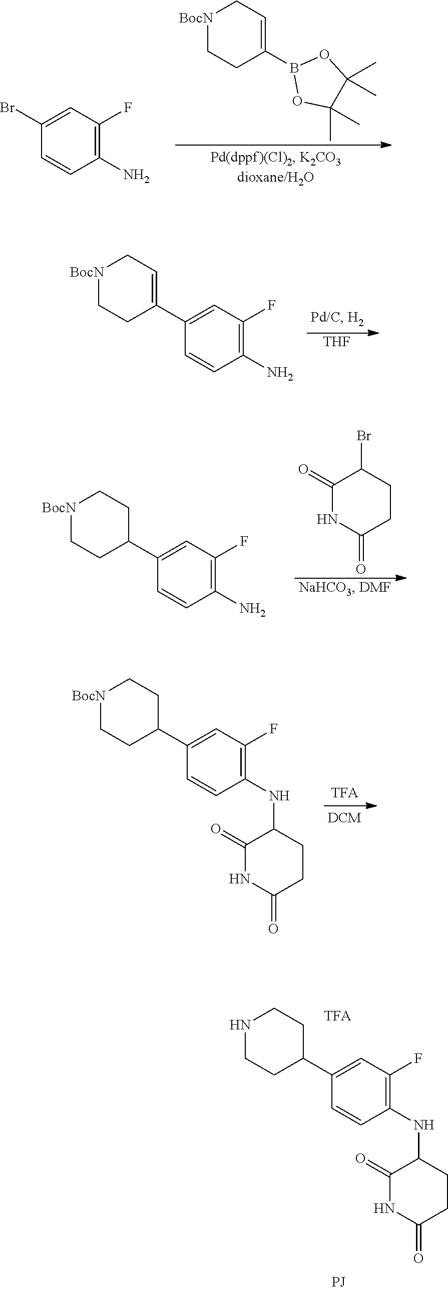

### Step 1—Tert-butyl 4-(4-amino-3-fluoro-phenyl)-3,6-dihydro-2H-pyridine-1-carboxylate

The mixture of 4-bromo-2-fluoro-aniline (10 g, 52.6 mmol, CAS #367-24-8), Pd(dppf)Cl₂ (3.85 g, 5.26 mmol) and K₂CO₃ (14.5 g, 105.0 mmol) in dioxane (100 mL) and H₂O (50 mL) was added tert-butyl 4-(4,4,5,5-tetramethyl-1,3,2-dioxaborolan-2-yl)-3,6-dihydro-2H-pyridine-1-carboxylate (16.2 g, 52.6 mmol, CAS #286961-14-6). The mixture was degassed with N₂ stream 3 times and then stirred at 90° C., in N₂ atmosphere for 12 hrs. On completion, the residue was diluted with water (200 mL), then the residue was extracted with EA (3×500 mL). The combined organic layer was dried over Na₂SO₄, filtered and concentrated in vacuo to give a residue. The residue was purified by column chromatography to give the title compound (14.7 g, 95% yield) as yellow oil. ¹H NMR (400 MHz, DMSO-d₆) δ 7.07 (dd, J=1.6, 13.2 Hz, 1H), 6.97 (dd, J=1.6, 8.4 Hz, 1H), 6.72 (t, J=8.8 Hz, 1H), 5.96 (s, 1H), 5.16 (s, 2H), 3.93 (d, J=8.4 Hz, 2H), 3.49 (t, J=5.6 Hz, 2H), 2.36 (d, J=1.2 Hz, 2H), 1.42 (s, 9H); LC-MS (ESI⁺) m/z 293.1 (M+H).

### Step 2—Tert-butyl 4-(4-amino-3-fluoro-phenyl)piperidine-1-carboxylate

To a solution of tert-butyl 4-(4-amino-3-fluoro-phenyl)-3,6-dihydro-2H-pyridine-1-carboxylate (13.0 g, 44.4 mmol) in THF (10 mL) was added Pd/C (12 g, 44.4 mmol, 10 wt %). The reaction mixture was stirred at 25° C. for 12 hrs under H₂ (15 Psi). On completion, the reaction mixture was filtered and filtrate was concentrated in vacuo to give the title compound (12.0 g, 91% yield) as yellow oil. ¹H NMR (400 MHz, DMSO-d₆) δ 6.84 (dd, J=1.6, 12.8 Hz, 1H), 6.73-6.67 (m, 2H), 4.89 (s, 2H), 4.03 (d, J=11.6 Hz, 2H), 2.74 (d, J=1.2 Hz, 2H), 1.68 (d, J=12.4 Hz, 2H), 1.40 (s, 9H).

### Step 3—Tert-butyl 4-[4-[(2,6-dioxo-3-piperidyl)amino]-3-fluoro-phenyl]piperidine-1-carboxylate

To a solution of tert-butyl 4-(4-amino-3-fluoro-phenyl)piperidine-1-carboxylate (2.00 g, 6.79 mmol) in DMF (30 mL) was added 3-bromopiperidine-2,6-dione (5.22 g, 27.1 mmol, CAS #62595-74-8) and NaHCO₃ (1.71 g, 20.3 mmol). The mixture was then stirred at 70° C. for 12 hrs. On completion, the residue was diluted with water (30 mL), then the residue was extracted with EA (2×50 mL). The combined organic layer was dried over Na₂SO₄, filtered and concentrated in vacuo to give a residue. The residue was purified by column chromatography to give the title compound (2.70 g, 98% yield) as yellow solid. LC-MS (ESI⁺) m/z 350.1 (M+H)⁺.

### Step 4—3-[2-Fluoro-4-(4-piperidyl)anilino]piperidine-2,6-dione

To a mixture of tert-butyl 4-[4-[(2,6-dioxo-3-piperidyl)amino]-3-fluoro-phenyl]piperidine-1-carboxylate (200 mg, 493 umol) in DCM (2 mL) was added TFA (56.2 mg, 493 umol, 36.5 uL). The mixture was stirred at 25° C. for 1 hr. On completion, the reaction mixture was concentrated in vacuo to give the title compound (200 mg, 96% yield, TFA) as brown oil. LC-MS (ESI⁺) m/z 306.1 (M+H)⁺.

### Synthesis of 3-[4-[1-[[4-[(2R)-2-aminopropoxy]cyclohexyl]methyl]-4-piperidyl]-2-fluoro-anilino]piperidine-2,6-dione (Intermediate PK)

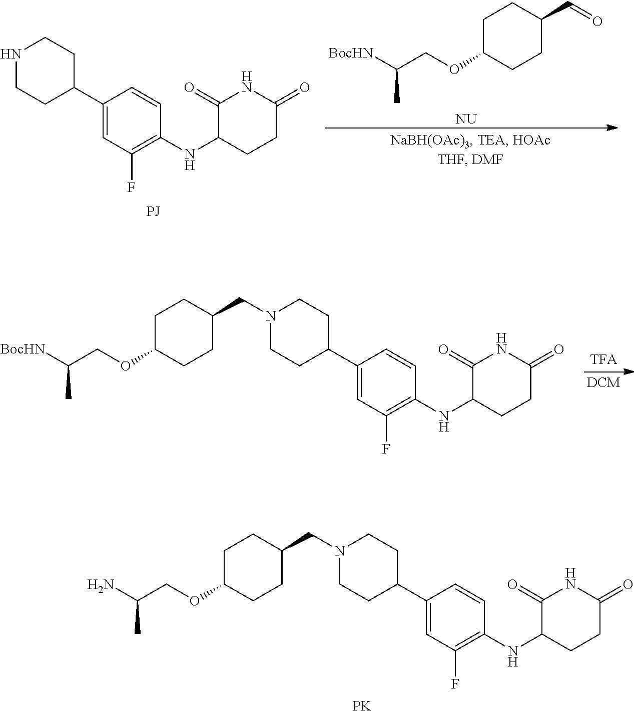 [SMILES: O=C1CCC(Nc2ccc(C3CCNCC3)cc2F)C(=O)N1 ; C[C@H](CO[C@H]1CC[C@H](C=O)CC1)NC(=O)OC(C)(C)C ; "NU" ; C[C@H](CO[C@H]1CC[C@H](CN2CCC(c3ccc(NC4CCC(=O)NC4=O)c(F)c3)CC2)CC1)NC(=O)OC(C)(C)C ; C[C@@H](N)CO[C@H]1CC[C@H](CN2CCC(c3ccc(NC4CCC(=O)NC4=O)c(F)c3)CC2)CC1 ; "PK"]

### Step 1—Tert-butyl N-[(1R)-2-[4-[[4-[4-[(2,6-dioxo-3-piperidyl)amino]-3-fluoro-phenyl]-1-piperidyl]methyl]cyclohexoxy]-1-methyl-ethyl]carbamate

To a mixture of 3-[2-fluoro-4-(4-piperidyl)anilino]piperidine-2,6-dione (50.0 mg, 119 μmol, TFA, Intermediate PJ) and TEA (12.0 mg, 119 μmol) in THF (1 mL) and DMF (0.5 mL) was added HOAc (7.16 mg, 119 μmol) and tert-butyl N-[(1R)-2-(4-formylcyclohexoxy)-1-methyl-ethyl]carbamate (34.0 mg, 119 mol, Intermediate NU) at −10° C. The reaction mixture was stirred at −10° C. for 0.5 hr, then NaBH(OAc)₃ (50.5 mg, 238 μmol) was added. The reaction mixture was stirred at −10° C. for 1 hr. On completion, the reaction mixture was quenched with water (0.5 mL) and concentrated in vacuo. The residue was purified by prep-HPLC (column: Welch Ultimate C18 150*25 mm*5 um; mobile phase: [water (FA)-ACN]; gradient: 15O %-45O % B over 10 min) to give the title compound (15.0 mg, 20% yield, FA) as white solid. ¹H NMR (400 MHz, DMSO-d₆) δ 10.82 (s, 1H), 6.93 (d, J=12.4 Hz, 1H), 6.87-6.80 (m, 1H), 6.79-6.72 (m, 1H), 6.61 (d, J=7.6 Hz, 1H), 5.45-5.37 (m, 1H), 4.40-4.31 (m, 1H), 3.58-3.47 (m, 1H), 3.20-3.11 (m, 2H), 3.05 (dd, J=2.8, 4.0 Hz, 2H), 2.82-2.71 (m, 1H), 2.58 (d, J=2.8 Hz, 2H), 2.47-2.38 (m, 2H), 2.33 (s, 2H), 2.25-2.15 (m, 1H), 2.11-2.00 (m, 2H), 2.00-1.90 (m, 2H), 1.76 (t, J=12.4 Hz, 4H), 1.69-1.58 (m, 2H), 1.57-1.46 (m, 1H), 1.37 (s, 9H), 1.17-1.04 (m, 2H), 0.99 (d, J=6.8 Hz, 3H), 0.94-0.82 (m, 2H); LC-MS (ESI⁺) m/z 575.4 (M+H)⁺.

### Step 2—3-[4-[1-[[4-[(2R)-2-aminopropoxy]cyclohexyl]methyl]-4-piperidyl]-2-fluoro-anilino]piperidine-2,6-dione

To a solution of tert-butyl N-[(1R)-2-[4-[[4-[4-[(2,6-dioxo-3-piperidyl)amino]-3-fluoro-phenyl]-1-piperidyl]methyl]cyclohexoxy]-1-methyl-ethyl]carbamate (140 mg, 243 μmol) in DCM (1 mL) was added TFA (1.54 g, 13.4 mmol) stirred at 25° C. for 0.5 hr. On completion, the reaction mixture was concentrated in vacuo to give the title compound (141 mg, 98% yield, TFA) as white solid. LC-MS (ESI⁺) m/z 475.1 (M+H).

### Synthesis of 3-[3-[4-[(4-Aminocyclohexyl)methyl]piperazin-1-yl]anilino]piperidine-2,6-dione (Intermediate PL)

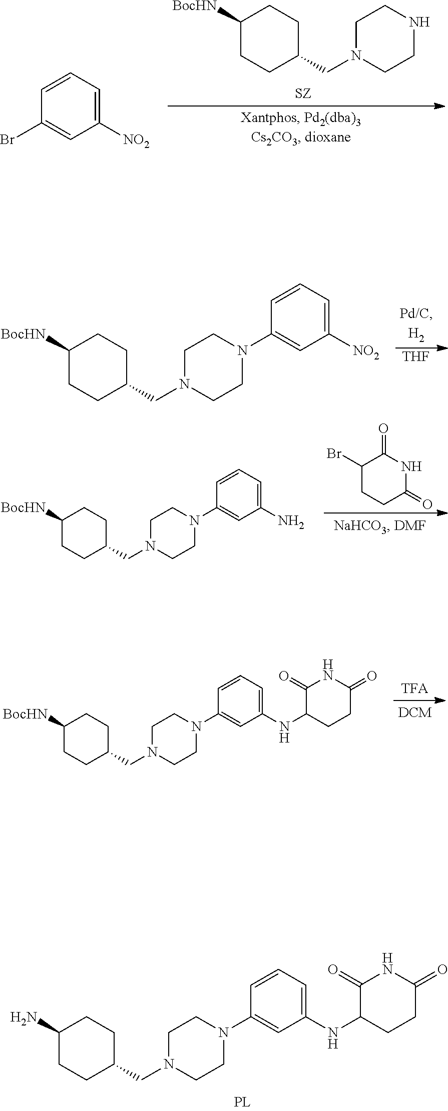

### Step 1—Tert-butyl N-[4-[[4-(3-nitrophenyl)piperazin-1-yl]methyl]cyclohexyl]carbamate

To a solution of 1-bromo-3-nitro-benzene (1 g, 4.95 mmol, CAS #585-79-5) and tert-butyl N-[4-(piperazin-1-ylmethyl)cyclohexyl]carbamate (1.62 g, 5.45 mmol, Intermediate SZ) in toluene (15 mL) was added (5-diphenylphosphanyl-9,9-dimethyl-xanthen-4-yl)-diphenyl-phosphane (143 mg, 247 μmol) and (1E,4E)-1,5-diphenylpenta-1,4-dien-3-one palladium (226 mg, 247 μmol) and Cs₂CO₃ (3.23 g, 9.90 mmol). The mixture was then stirred at 110° C. under N₂ for 12 hrs. On completion, the mixture was added H₂O (50 ml) and extracted with EA mL (30 mL×3). The organic layers were dried over Na₂SO₄, filtered and concentrated under reduced pressure to give a residue. The residue was purified by column chromatography (SiO₂, PE:EA=10:1 to 1:1) to give the title compound (800 mg, 38% yield) as yellow oil. ¹H NMR (400 MHz, DMSO-d₆) δ 7.66-7.62 (m, 1H), 7.58 (d, J=7.6 Hz, 1H), 7.47 (t, J=8.0 Hz, 1H), 7.41-7.37 (m, 1H), 6.71 (d, J=8.0 Hz, 1H), 3.20-3.11 (m, 1H), 2.48-2.45 (m, 4H), 2.12 (d, J=7.2 Hz, 2H), 1.77 (d, J=10.8 Hz, 4H), 1.38 (s, 14H), 1.15-1.07 (m, 2H), 0.88 (d, J=10.8 Hz, 2H).

### Step 2—Tert-butylN-[4-[[4-(3-aminophenyl)piperazin-1-yl]methyl]cyclohexyl]carbamate

To a solution of tert-butyl N-[4-[[4-(3-nitrophenyl)piperazin-1-yl]methyl]cyclohexyl]carbamate (800 mg, 1.91 mmol) in THF (10 mL) was added Pd/C (571 mg, 536 μmol) under N₂. The suspension was degassed under vacuum and purged with H₂ several times. The mixture was stirred under H₂ (15 psi) at 25° C. for 6 hrs. On completion, the mixture was filtered and concentrated in vacuo to give the title compound (700 mg, 94% yield) as yellow solid. ¹H NMR (400 MHz, DMSO-d₆) δ 6.90-6.77 (m, 1H), 6.71 (d, J=8.0 Hz, 1H), 6.19-6.08 (m, 2H), 6.02 (dd, J=1.2, 7.6 Hz, 1H), 4.84 (s, 2H), 3.60-3.58 (m, 2H), 3.32 (s, 1H), 3.16 (d, J=8.4 Hz, 1H), 3.07-2.92 (m, 4H), 2.45-2.34 (m, 4H), 2.09 (d, J=7.2 Hz, 2H), 1.43-1.33 (m, 11H), 1.12 (d, J=12.0 Hz, 2H), 0.86 (d, J=12.0 Hz, 2H); LC-MS (ESI⁺) m/z 389.2 (M+H)⁺.

### Step 3—Tert-butyl N-[4-[[4-[3-[(2,6-dioxo-3-piperidyl)amino]phenyl]piperazin-1-yl]methyl]cyclohexyl]carbamate

To a solution of tert-butyl N-[4-[[4-(3-aminophenyl)piperazin-1-yl]methyl]cyclohexyl]carbamate (500 mg, 1.29 mmol) and 3-bromopiperidine-2,6-dione (345 mg, 1.80 mmol, CAS #62595-74-8) in DMF (6 mL) was added NaHCO₃ (216 mg, 2.57 mmol). The mixture was then stirred at 80° C. for 16 hrs. On completion, the reaction mixture was diluted with H₂O (50 mL) and extracted with EA (30 mL×3). The combined organic layers were dried over Na₂SO₄, filtered and concentrated under reduced pressure. The residue was purified by prep-HPLC (column: Phenomenex luna C18 150*25 mm*10 um; mobile phase: [water (FA)-ACN]; gradient: 7%-37% B over 10 min) to give the title compound (180 mg, 28% yield) as yellow solid. LC-MS (ESI⁺) m/z 500.2 (M+H)⁺.

### Step 4—3-[3-[4-[(4-Aminocyclohexyl)methyl]piperazin-1-yl]anilino]piperidine-2,6-dione

To a solution of tert-butyl N-[4-[[4-[3-[(2,6-dioxo-3-piperidyl)amino]phenyl]piperazin-1-yl]methyl] cyclohexyl]carbamate (80 mg, 160 μmol) in DCM (1 mL) was added TFA (736 mg, 6.46 mmol). The mixture was then stirred at 25° C. for 1 hr. On completion, the mixture was concentrated in vacuo to give the title compound (80 mg, 97% yield) as yellow oil. LC-MS (ESI⁺) m/z 400.1 (M+H)⁺.

### Synthesis of Tert-butyl N-[(3S,4R)-3-fluoro-4-piperidyl]carbamate (Intermediate PM)

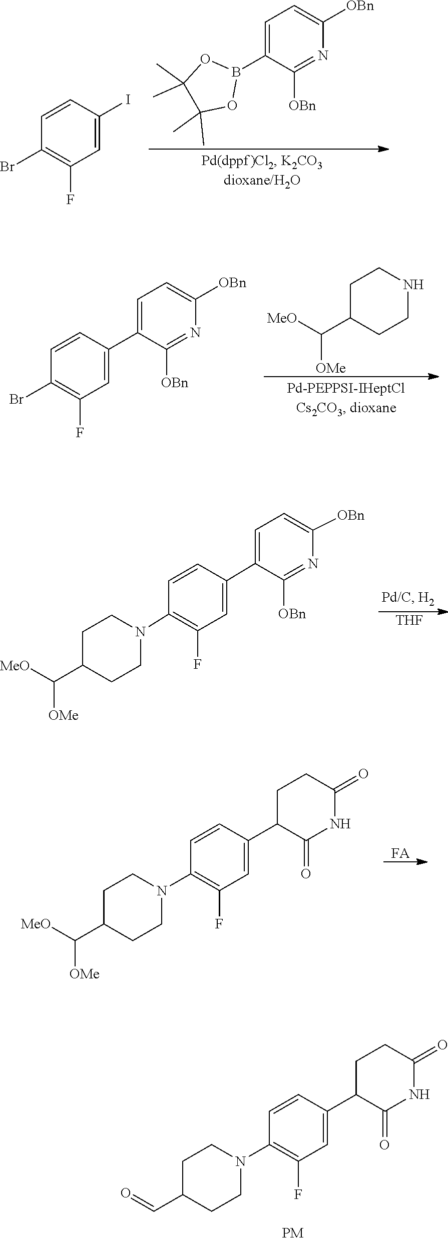

### Step 1—2,6-Dibenzyloxy-3-(4-bromo-3-fluoro-phenyl)pyridine

A mixture of 1-bromo-2-fluoro-4-iodo-benzene (5 g, 16.6 mmol, CAS #136434-77-0), 2,6-dibenzyloxy-3-(4,4,5,5-tetramethyl-1,3,2-dioxaborolan-2-yl)pyridine (5.55 g, 13.29 mmol, CAS#2152673-80-6), Pd(dppf)Cl₂·CH₂Cl₂ (1.36 g, 1.66 mmol), and K₂CO₃ (6.89 g, 49.8 mmol) in dioxane (45 mL) and H₂O (15 mL) was degassed and purged with N₂ for 3 time. Then the mixture was stirred at 80° C. for 2 hrs under N₂ atmosphere. On completion, the mixture was diluted with EA (200 mL). The organic layer was washed with water (80 mL×2), dried over Na₂SO₄ and concentrated in vacuo. The residue was purified by column chromatography (SiO₂, Petroleum ether/Ethyl acetate=15:1 to 10:1) to give the title compound (5.3 g, 68% yield) as white oil. LC-MS (ESI⁺) m/z 465.9 (M+H)⁺.

### Step 2—2,6-Dibenzyloxy-3-[4-[4-(dimethoxymethyl)-1-piperidyl]-3-fluoro-phenyl]pyridine

A mixture of 2,6-dibenzyloxy-3-(4-bromo-3-fluoro-phenyl)pyridine (300 mg, 646 μmol), 4-(dimethoxymethyl)piperidine (154 mg, 969 μmol), Cs₂CO₃ (631 mg, 1.94 mmol) and Pd-PEPPSI-IHeptCl (62.8 mg, 64.6 μmol) in dioxane (5 mL) was stirred at 100° C. for 16 hrs under N₂. On completion, the reaction mixture was diluted with EA (100 mL) and filtered. The filtrate was concentrated in vacuo. The residue was purified by column chromatography (SiO₂, EA in PE, 0% to 10%) to give the title compound (214 mg, 61% yield) as yellow oil. ¹H NMR (400 MHz, CDCl₃) δ=7.59 (d, J=8.0 Hz, 1H), 7.46-7.27 (m, 11H), 7.25 (d, J=2.0 Hz, 1H), 6.96 (t, J=8.8 Hz, 1H), 6.46 (d, J=8.0 Hz, 1H), 5.46-5.35 (m, 4H), 4.12 (d, J=7.2 Hz, 1H), 3.56-3.48 (m, 2H), 3.39 (s, 6H), 2.73-2.60 (m, 2H), 1.86 (d, J=12.8 Hz, 2H), 1.83-1.71 (m, 1H), 1.62-1.57 (m, 1H), 1.56-1.50 (m, 1H); LC-MS (ESI⁺) m/z 543.5 (M+H)⁺.

### Step 3—3-[4-[4-(Dimethoxymethyl)-1-piperidyl]-3-fluoro-phenyl]piperidine-2,6-dione

To a solution of 2,6-dibenzyloxy-3-[4-[4-(dimethoxymethyl)-1-piperidyl]-3-fluoro-phenyl]pyridine (214 mg, 394 μmol) in THF (10 mL) was added Pd/C (200 mg, 187 μmol, 10 wt %) under Ar. The suspension was degassed under vacuum and purged with H₂ several times. The mixture was stirred under H₂ (15 Psi) at rt for 16 hrs. On completion, the reaction mixture was filtered and the filtrate was concentrated in vacuo to give the title compound (130 mg, 90% yield) as off-white solid. LC-MS (ESI⁺) m/z 365.0 (M+H)⁺.

### Step 4—1-[4-(2,6-Dioxo-3-piperidyl)-2-fluoro-phenyl]piperidine-4-carbaldehyde

A mixture of 3-[4-[4-(dimethoxymethyl)-1-piperidyl]-3-fluoro-phenyl]piperidine-2,6-dione (42 mg, 115 mol) in HCOOH (1 mL) was stirred at 80° C. for 0.5 hr. On completion, the reaction mixture was concentrated in vacuo to give the title compound (36.6 mg, 99% yield) as brown solid. LC-MS (ESI⁺) m z 319.1 (M+H)⁺.

### Synthesis of 3-[4-[4-[[(3S,4R)-4-amino-3-fluoro-1-piperidyl]methyl]-1-piperidyl]-3-fluoro-phenyl]piperidine-2,6-dione (Intermediate PN)

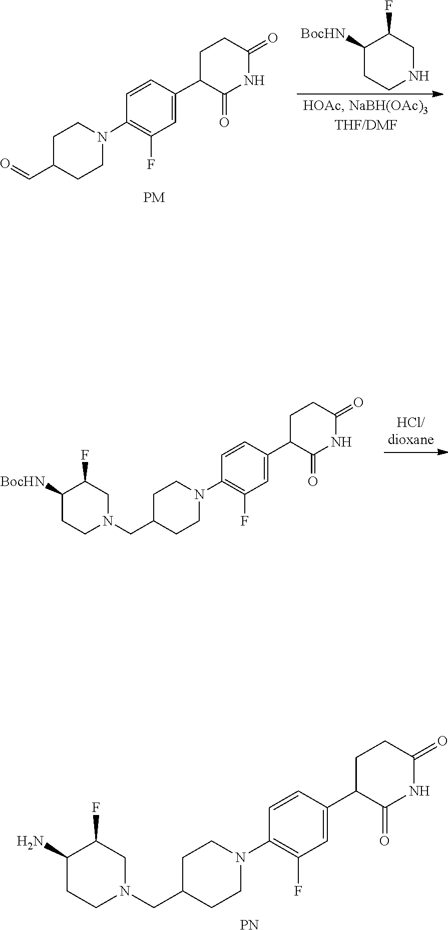

### Step 1—Tert-butyl N-[(3S,4R)-1-[[1-[4-(2,6-dioxo-3-piperidyl)-2-fluoro-phenyl]-4-piperidyl]methyl]-3-fluoro-4-piperidyl]carbamate

To a solution of tert-butyl N-[(3S,4R)-3-fluoro-4-piperidyl]carbamate (85.0 mg, 389 μmol, Intermediate PM) in THF (3 mL) and DMF (0.5 mL) was added TEA (389 μmol, 54.2 μL), then 1-[4-(2,6-dioxo-3-piperidyl)-2-fluoro-phenyl]piperidine-4-carbaldehyde (124 mg, 389 μmol, CAS #1434126-99-4) and AcOH (389 mol, 22.3 μL) were added. After 0.2 hour of stirring, NaBH(OAc)₃ (165 mg, 778 μmol) was added. The mixture was then stirred at 25° C. for 1 hr. On completion, the mixture was concentrated in vacuo to give the residue. The residue was purified by prep-HPLC (column: Phenomenex luna C18 150*25 mm*10 um; mobile phase: [water (FA)-ACN]; gradient:2%-32% B over 11 min) to give the title compound (90.0 mg, 44% yield) as a white solid. ¹H NMR (400 MHz, DMSO-d₆) δ 10.81 (s, 1H), 7.04-6.96 (m, 2H), 6.96-6.90 (m, 2H), 4.72-4.51 (m, 1H), 3.84-3.74 (m, 1H), 3.08-2.98 (m, 1H), 2.79 (d, J=10.4 Hz, 1H), 2.69-2.57 (m, 5H), 2.35-2.30 (m, 1H), 2.24-2.12 (m, 4H), 2.09-1.95 (m, 3H), 1.75 (t, J=9.2 Hz, 3H), 1.65-1.56 (m, 1H), 1.54-1.46 (m, 1H), 1.39 (s, 9H), 1.29-1.17 (m, 2H). LC-MS (ESI⁺) m/z 521.1 (M+H)⁺.

### Step 2—3-[4-[4-[[(3S,4R)-4-amino-3-fluoro-1-piperidyl]methyl]-1-piperidyl]-3-fluoro-phenyl]piperidine-2,6-dione

A solution of tert-butyl N-[(3S,4R)-1-[[1-[4-(2,6-dioxo-3-piperidyl)-2-fluoro-phenyl]-4-piperidyl]methyl]-3-fluoro-4-piperidyl]carbamate (70.0 mg, 134 μmol,) in HCl/dioxane (4 mL) was stirred 25° C. for 0.5 hr. On completion, the mixture concentrated in vacuo to give the title compound (55 mg, 89% yield, HCl) as a white solid. LC-MS (ESI⁺) m/z 421.0 (M+H)⁺.

### Synthesis of 3-(2-Fluoro-4-(piperazin-1-yl)phenyl)piperidine-2,6-dione (Intermediate PO)

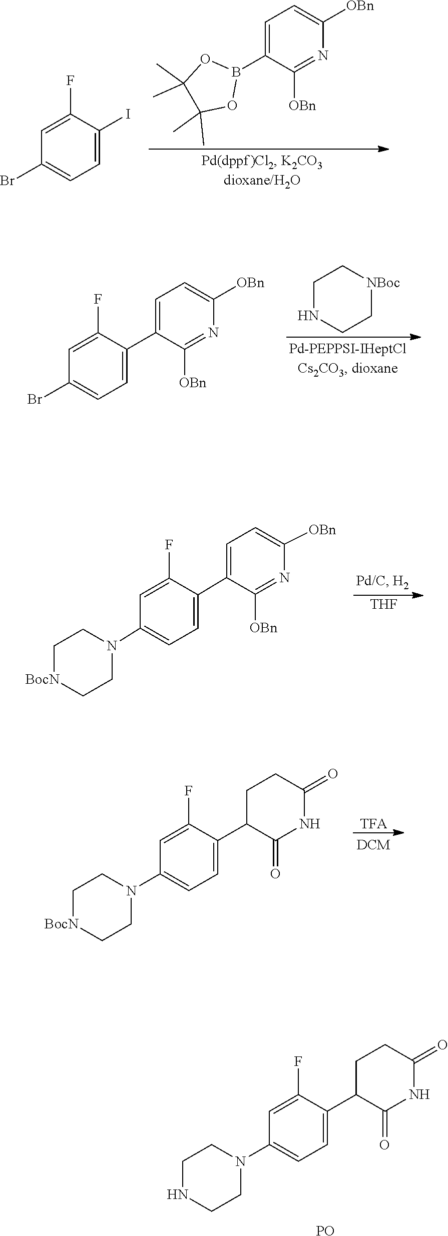

### Step 1—2,6-Bis(benzyloxy)-3-(4-bromo-2-fluorophenyl)pyridine

To a mixture of 4-bromo-2-fluoro-1-iodo-benzene (2 g, 6.65 mmol, CAS #105931-73-5) and 2,6-dibenzyloxy-3-(4,4,5,5-tetramethyl-1,3,2-dioxaborolan-2-yl)pyridine (3.05 g, 7.31 mmol, CAS #2152673-80-6) in dioxane (20 mL) and H₂O (4 mL) were added K₂CO₃ (2.76 g, 19.9 mmol) and Pd(dppf)Cl₂·CH₂Cl₂ (542 mg, 664 μmol). The mixture was then stirred for 2 hrs at 80° C. under N₂. On completion, the reaction was diluted with H₂O (60 mL) and extracted with EtOAc (100 mL). The organic layer was washed with brine (60 mL), dried over Na₂SO₄, filtered and concentrated in vacuo. The residue was purified by flash silica gel chromatography (ISCO®; 40 g SepaFlash® Silica Flash Column, Eluent of 0˜8% Ethyl acetate/Petroleum ethergradient @ 100 mL/min) to give the title compound (2 g, 59% yield) as colorless oil. H NMR (400 MHz, DMSO-d₆) δ 7.70-7.55 (m, 2H), 7.46-7.27 (m, 12H), 6.56 (d, J=8.0 Hz, 1H), 5.37 (s, 4H).

### Step 2—Tert-butyl 4-(4-(2,6-bis(benzyloxy)pyridin-3-yl)-3-fluorophenyl)piperazine-1-carboxylate

To a mixture of 2,6-dibenzyloxy-3-(4-bromo-2-fluoro-phenyl)pyridine (1.95 g, 4.20 mmol) and tert-butyl piperazine-1-carboxylate (860 mg, 4.62 mmol, CAS #57260-71-6) in dioxane (25 mL) was added Cs₂CO₃ (4.10 g, 12.6 mmol) and 1,3-bis[2,6-bis(1-propylbutyl) phenyl]-4,5-dichloro-2H-imidazol-1-ium-2-ide 3-chloropyridine dichloropalladium (408 mg, 419 μmol) under N₂. The mixture was stirred at 100° C. for 4 hrs. On completion, the reaction was diluted with H₂O (40 mL) and extracted with EtOAc (80 mL). The organic layer was washed with brine (40 mL), dried over with Na₂SO₄, filtered and concentrated in vacuo. The residue was purified by flash silica gel chromatography (ISCO®; 40 g SepaFlash® Silica Flash Column, Eluent of 0˜4% Ethyl acetate/Petroleum ethergradient @ 100 mL/min) to give the title compound (1.08 g, 44% yield) as yellow oil. ¹H NMR (400 MHz, CDCl₃) δ 7.53 (m, 1H), 7.46-7.25 (m, 11H), 6.77-6.63 (m, 2H), 6.46 (d, J=8.0 Hz, 1H), 5.41 (s, 2H), 5.35 (s, 2H), 3.68-3.49 (m, 4H), 3.26-3.13 (m, 4H), 1.50 (s, 9H).

### Step 3—Tert-butyl 4-(4-(2,6-dioxopiperidin-3-yl)-3-fluorophenyl)piperazine-1-carboxylate

To a solution of tert-butyl 4-[4-(2,6-dibenzyloxy-3-pyridyl)-3-fluoro-phenyl]piperazine-1-carboxylate (1.08 g, 1.90 mmol) in THF (20 mL) was added Pd/C (1 g, 939 μmol, 10 wt %) under Ar₂. The suspension was degassed under vacuum and purged with H₂ several times. The mixture was stirred under H₂ (15 Psi) at 20° C. for 12 hrs. On completion, the mixture was filtered and concentrated in vacuo to give the title compound (670 mg, 90% yield) as white solid. ¹H NMR (400 MHz, DMSO-d₆) δ 10.81 (s, 1H), 7.11 (t, J=8.8 Hz, 1H), 6.81-6.70 (m, 2H), 3.89 (dd, J=4.8, 12.4 Hz, 1H), 3.51-3.39 (m, 4H), 3.20-3.06 (m, 4H), 2.78-2.63 (m, 1H), 2.52 (s, 1H), 2.14 (m, 1H), 2.02-1.87 (m, 1H), 1.42 (s, 9H).

### Step 4—3-(2-Fluoro-4-(piperazin-1-yl)phenyl)piperidine-2,6-dione

A solution of tert-butyl 4-[4-(2,6-dioxo-3-piperidyl)-3-fluoro-phenyl]piperazine-1-carboxylate (200 mg, 510 μmol) in CH₂Cl₂ (2 mL) and TFA (0.5 mL) was stirred at 20° C. for 0.5 hr. On completion, the reaction was concentrated in vacuo to give the title compound (207 mg, 99% yield) as colorless oil. LC-MS (ESI⁺) m/z 292.0 (M+H)⁺.

### Synthesis of 3-(4-(4-(((1r,4r)-4-aminocyclohexyl)methyl)piperazin-1-yl)-2-fluorophenyl) piperidine-2,6-dione (Intermediate PP)

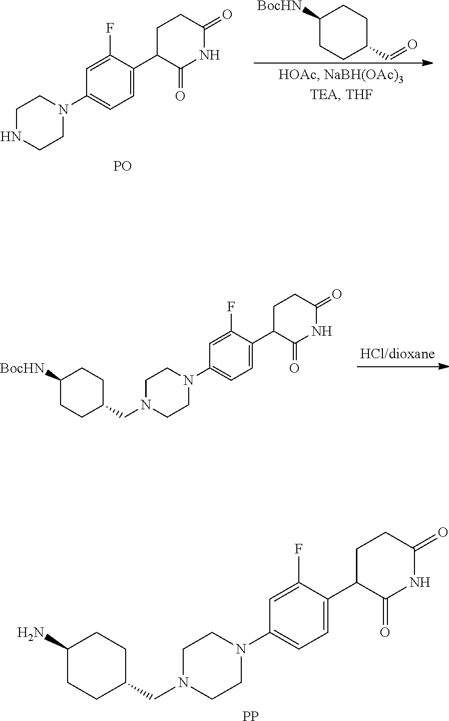

### Step 1—Tert-butyl ((1r,4r)-4-((4-(4-(2,6-dioxopiperidin-3-yl)-3-fluorophenyl)piperazin-1-yl) methyl)cyclohexyl)carbamate

To a solution of 3-(2-fluoro-4-piperazin-1-yl-phenyl)piperidine-2,6-dione (200 mg, 686 μmol, Intermediate PO) in THF (5 mL) was added TEA (208 mg, 2.06 mmol, 286 μL), HOAc (61.8 mg, 1.03 mmol, 58.9 μL) and tert-butyl N-(4-formylcyclohexyl)carbamate (156 mg, 686 μmol, CAS #181308-57-6). The mixture was stirred at 20° C. for 0.5 hr. Then, NaBH(OAc)₃ (291 mg, 1.37 mmol) was added at −10° C., and the mixture was stirred at −10° C. for 1 hr. On completion, the reaction was diluted with H₂O (20 mL) and extracted with EtOAc (60 mL×2). The combined organic layers were washed with brine (20 mL), dried over with Na₂SO₄, filtered and concentrated in vacuo. The residue was purified by prep-HPLC (column: Phenomenex luna C18 150*25 mm*10 um; mobile phase: [water (FA)-ACN]; gradient: 10%-40% B over 10 min) to give the title compound (130 mg, 37% yield) as white solid. ¹H NMR (400 MHz, DMSO-d₆) δ 10.82 (s, 1H), 7.23-7.00 (m, 1H), 6.97-6.60 (m, 3H), 3.97-3.76 (m, 2H), 3.57 (d, J=5.2 Hz, 1H), 3.23-2.98 (m, 6H), 2.72-2.60 (m, 3H), 2.24-2.04 (m, 2H), 1.98-1.90 (m, 1H), 1.78 (d, J=10.4 Hz, 5H), 1.37 (s, 10H), 1.25-1.09 (m, 2H), 1.07-0.78 (m, 2H); LC-MS (ESI⁺) m/z 503.0 (M+H)⁺.

### Step 2-3-(4-(4-(((1r,4r)-4-aminocyclohexyl)methyl)piperazin-1-yl)-2-fluorophenyl) piperidine-2,6-dione

A solution of tert-butyl N-[4-[[4-[4-(2,6-dioxo-3-piperidyl)-3-fluoro-phenyl]piperazin-1-yl]methyl]cyclohexyl]carbamate (60 mg, 119 μmol) in HCl/dioxane (1 mL, 4M) was stirred at 20° C. for 1 hr. On completion, the mixture was concentrated in vacuo to give the title compound (61 mg, 99% yield) as white solid. LC-MS (ESI⁺) m/z 403.1 (M+H)⁺.

### Synthesis of 3-[2-Chloro-4-[(3S)-3-methylpiperazin-1-yl]phenyl]piperidine-2,6-dione (Intermediate PQ)

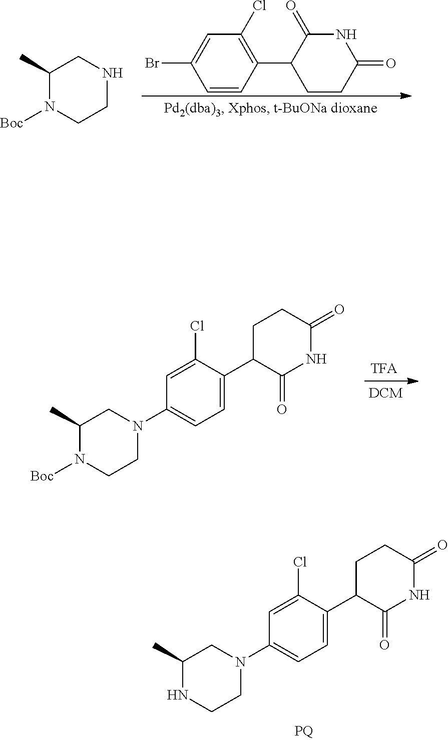 [SMILES: C[C@H]1CNCCN1C(=O)OC(C)(C)C ; O=C1CCC(c2ccc(Br)cc2Cl)C(=O)N1 ; C[C@H]1CN(c2ccc(C3CCC(=O)NC3=O)c(Cl)c2)CCN1C(=O)OC(C)(C)C ; C[C@H]1CN(c2ccc(C3CCC(=O)NC3=O)c(Cl)c2)CCN1]

### Step 1—Tert-butyl (2S)-4-[3-chloro-4-(2,6-dioxo-3-piperidyl)phenyl]-2-methyl-piperazine-1-carboxylate

To a solution of tert-butyl (2S)-2-methylpiperazine-1-carboxylate (695 mg, 3.47 mmol, CAS #469447-70-5), 3-(4-bromo-2-chloro-phenyl)piperidine-2,6-dione (700 mg, 2.31 mmol, synthesized via Steps 1-2 of Intermediate PH), t-BuONa (667 mg, 6.94 mmol) and 4A molecular sieves (100 mg) in dioxane (20 mL) was added XPhos (110 mg, 231 mol) and Pd₂(dba)₃ (211 mg, 231 mol). The reaction was stirred at 100° C. for 3 hrs under N₂. On completion, the reaction was diluted with EA (60 mL). The organic layer was washed with water (60 mL×2), dried over Na₂SO₄ and concentrated in vacuo. The residue was purified by column chromatography (SiO₂, PE/EA=10/1 to 1/1) to give the title compound (500 mg, 51% yield) as yellow solid. ¹H NMR (400 MHz, DMSO-d₆) δ 10.82 (s, 1H), 7.13 (d, J=8.4 Hz, 1H), 6.94 (d, J=2.4 Hz, 1H), 6.89-6.85 (m, J=2.4, 8.4 Hz, 1H), 4.23-4.14 (m, 1H), 4.07-4.03 (m, 1H), 3.80-3.74 (m, 1H), 3.60 (d, J=12.0 Hz, 1H), 3.51 (d, J=12.4 Hz, 1H), 3.20-3.08 (m, 1H), 2.91-2.84 (m, 1H), 2.77-2.62 (m, 2H), 2.29-2.17 (m, 1H), 1.99 (s, 1H), 1.96-1.89 (m, 1H), 1.42 (s, 9H), 1.17 (d, J=6.4 Hz, 3H); LC-MS (ESI⁺) m/z 422.2 (M+H)⁺.

### Step 2—3-[2-Chloro-4-[(3S)-3-methylpiperazin-1-yl]phenyl]piperidine-2,6-dione

To a solution of tert-butyl (2S)-4-[3-chloro-4-(2,6-dioxo-3-piperidyl)phenyl]-2-methyl-piperazine-1-carboxylate (350 mg, 829 μmol) in DCM (3 mL) was added TFA (1.54 g, 13.4 mmol, 1 mL). The reaction was stirred at 25° C. for 0.5 hr. On completion the reaction was concentrated in vacuo to give the title compound (361 mg, 99% yield, TFA) as brown oil. LC-MS (ESI⁺) m/z 322.0 (M+H)⁺.

### Synthesis of 3-[4-[(3S)-4-[(4-amino-2-oxabicyclo[2.2.2]octan-1-yl)methyl]-3-methyl-piperazin-1-yl]-2-chloro-phenyl]piperidine-2,6-dione (Intermediate PR)

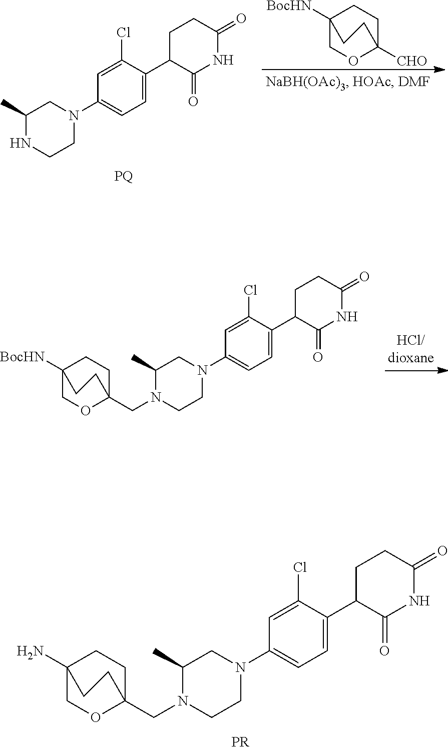 [SMILES: C[C@H]1CN(c2ccc(C3CCC(=O)NC3=O)c(Cl)c2)CCN1 ; CC(C)(C)OC(=O)NC12CCC(C=O)(CC1)OC2 ; C[C@H]1CN(c2ccc(C3CCC(=O)NC3=O)c(Cl)c2)CCN1CC12CCC(NC(=O)OC(C)(C)C)(CC1)CO2 ; C[C@H]1CN(c2ccc(C3CCC(=O)NC3=O)c(Cl)c2)CCN1CC12CCC(N)(CC1)CO2]

### Step 1—Tert-butyl N-[1-[[(2S)-4-[3-chloro-4-(2,6-dioxo-3-piperidyl)phenyl]-2-methyl-piperazin -1-yl]methyl]-2-oxabicyclo[2.2.2]octan-4-yl]carbamate

To a solution of 3-[2-chloro-4-[(3S)-3-methylpiperazin-1-yl]phenyl]piperidine-2,6-dione (361 mg, 828 μmol, TFA, Intermediate PQ) in DMF (5 mL) was added TEA (83.8 mg, 828 μmol, 115 μL) until the pH=8. The mixture was then stirred at 25° C. for 10 mins, then HOAc (49.7 mg, 828 mol, 47.4 μL) was added until the pH=6. Subsequently, tert-butyl N-(1-formyl-2-oxabicyclo[2.2.2]octan-4-yl)carbamate (232 mg, 911 mol, CAS #1417551-42-8) was added. The mixture was stirred at 50° C. for 20 mins. After that, NaBH(OAc)₃ (263 mg, 1.24 mmol) was added one portion. The resulting mixture was stirred at 25° C. for 30 mins. On completion, the reaction was concentrated in vacuo. The residue was purified by prep-HPLC (column: Welch Ultimate C18 150*25 mm*5 um; mobile phase: [water (FA)-ACN]; gradient: 15% -45% B over 10 min) to give the title compound (350 mg, 75% yield) as white solid. ¹H NMR (400 MHz, DMSO-d₆) δ 10.84 (s, 1H), 7.25-6.46 (m, 5H), 4.11-4.00 (m, 1H), 3.93-3.79 (m, 2H), 3.74 (s, 2H), 3.39 (s, 2H), 3.13 (d, J=3.2 Hz, 1H), 2.78-2.65 (m, 2H), 2.29-2.18 (m, 1H), 1.99-1.88 (m, 4H), 1.83-1.65 (m, 6H), 1.62-1.50 (m, 2H), 1.36 (s, 12H); LC-MS (ESI⁺) m/z 561.2 (M+H)⁺.

### Step 2—3-[4-[(3S)-4-[(4-amino-2-oxabicyclo[2.2.2]octan-1-yl)methyl]-3-methyl-piperazin-1-yl]-2-chloro-phenyl]piperidine-2,6-dione

To a solution of tert-butyl N-[1-[[(2S)-4-[3-chloro-4-(2,6-dioxo-3-piperidyl)phenyl]-2-methyl-piperazin-1-yl]methyl]-2-oxabicyclo[2.2.2]octan-4-yl]carbamate (80 mg, 142 μmol) in DCM (0.5 mL) was added HCl/dioxane (4 M, 200 μL). The reaction was stirred at 40° C. for 0.5 hr. On completion, the reaction was concentrated in vacuo to give the title compound (70.9 mg, 99% yield, HCl) as yellow solid. LC-MS (ESI⁺) m/z 461.2 (M+H)⁺.

### Synthesis of 3-[2-chloro-4-[(3R)-3-methylpiperazin-1-yl]phenyl]piperidine-2,6-dione (Intermediate PS)

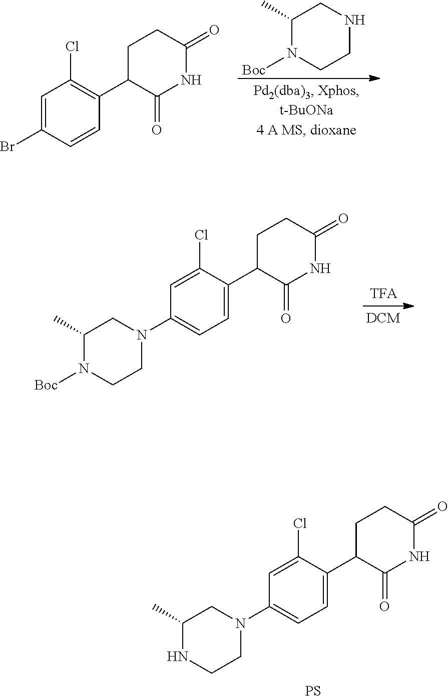 [SMILES: O=C1CCC(c2ccc(Br)cc2Cl)C(=O)N1 ; C[C@@H]1CNCCN1C(=O)OC(C)(C)C ; C[C@@H]1CN(c2ccc(C3CCC(=O)NC3=O)c(Cl)c2)CCN1C(=O)OC(C)(C)C ; C[C@@H]1CN(c2ccc(C3CCC(=O)NC3=O)c(Cl)c2)CCN1 ; "PS"]

### Step 1—Tert-butyl(2R)-4-[3-chloro-4-(2,6-dioxo-3-piperidyl)phenyl]-2-methyl-piperazine-1-carboxylate

To a solution of tert-butyl (2R)-2-methylpiperazine-1-carboxylate (1.19 g, 5.95 mmol, CAS #170033-47-3), 3-(4-bromo-2-chloro-phenyl)piperidine-2,6-dione (1.2 g, 3.97 mmol, synthesized via Steps 1-2 of Intermediate PH) in dioxane (20 mL) was added Pd₂(dba)₃ (363 mg, 396 mol), XPhos (189 mg, 396 μmol), t-BuONa (1.14 g, 11.9 mmol), and 4A molecular sieves (200 mg). Then the mixture was stirred at 100° C. for 3 hrs under N₂ atmosphere. On completion, the residue was diluted with water (30 mL), then extracted with EA (3×50 mL). The combined organic layers were dried over Na₂SO₄, filtered and concentrated in vacuo. The residue was purified by column chromatography (SiO₂, PE:EA=50:1 to PE:EA=2:1, PE:EA=1:1, PL:Rf=0.34) to give the title compound (790 mg, 47% yield) as yellow solid. ¹H NMR (400 MHz, DMSO-d₆) δ 10.82 (s, 1H), 7.13 (d, J=8.4 Hz, 1H), 6.93 (d, J=2.4 Hz, 1H), 6.87 (dd, J=2.4, 8.4 Hz, 1H), 4.23-4.14 (m, 1H), 4.10-4.00 (m, 1H), 3.78 (d, J=13.2 Hz, 1H), 3.60 (d, J=12.0 Hz, 1H), 3.51 (d, J=12.0 Hz, 1H), 3.20-3.08 (m, 1H), 2.87 (dd, J=3.6, 12.0 Hz, 1H), 2.80-2.62 (m, 2H), 2.27-2.18 (m, 1H), 2.00-1.88 (m, 2H), 1.42 (s, 9H), 1.20-1.14 (m, 3H); LC-MS (ESI⁺) m/z 421.9 (M+H)⁺.

### Step 2—3-[2-Chloro-4-[(3R)-3-methylpiperazin-1-yl]phenyl]piperidine-2,6-dione

To a solution of tert-butyl (2R)-4-[3-chloro-4-(2,6-dioxo-3-piperidyl)phenyl]-2-methyl-piperazine-1-carboxylate (30 mg, 71.1 μmol) in DCM (1 mL) was added TFA (383 mg, 3.37 mmol, 0.25 mL), then the mixture was stirred at 25° C. for 0.5 hr. On completion, the reaction mixture was concentrated in vacuo to give the title compound (30 mg, 96% yield, TFA) as yellow oil. LC-MS (ESI⁺) m/z 322.0 (M+H)⁺.

### Synthesis of 3-[4-[(3R)-4-[(4-amino-2-oxabicyclo[2.2.2]octan-1-yl)methyl]-3-methyl-piperazin-1-yl]-2-chloro-phenyl]piperidine-2,6-dione (Intermediate PT)

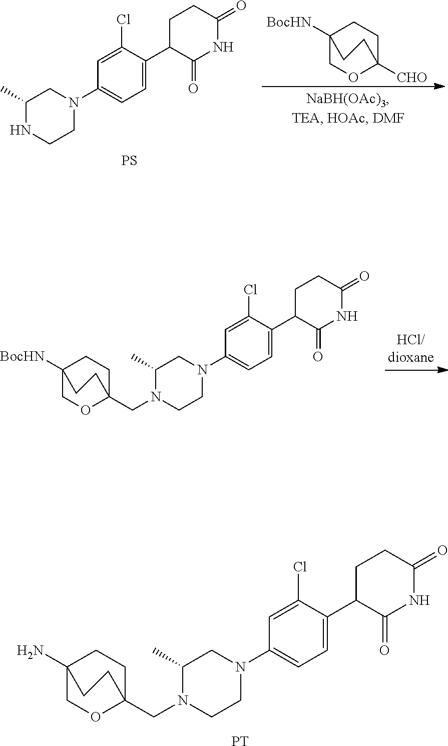

### Step 1—Tert-butyl N-[1-[[(2R)-4-[3-chloro-4-(2,6-dioxo-3-piperidyl)phenyl]-2-methyl-piperazin-1-yl]methyl]-2-oxabicyclo[2.2.2]octan-4-yl]carbamate

To a solution of 3-[2-chloro-4-[(3R)-3-methylpiperazin-1-yl]phenyl]piperidine-2,6-dione (360 mg, 826 mol, TFA, Intermediate PS) in DMF (3 mL) was added TEA (83.5 mg, 826 μmol, 114 μL) until the pH=8-10. Then tert-butyl N-(1-formyl-2-oxabicyclo[2.2.2]octan-4-yl)carbamate (231 mg, 908 μmol, CAS #1417551-42-8) in DMF (3 mL) and AcOH (49.6 mg, 826 μmol, 47.2 μL) was added to the mixture and the mixture was stirred at 25° C. for 0.5 hr. Then, NaBH(OAc)₃ (350 mg, 1.65 mmol) was added and the mixture was stirred at 25° C. for 1 hr. On completion, the reaction mixture was concentrated in vacuo, the residue was purified by prep-HPLC (column: Welch Ultimate C18 150*25 mm*5 um; mobile phase: [water (FA)-ACN]; gradient: 15%-45% B over 10 min) to give the title compound (238 mg, 51% yield) as yellow solid. ¹H NMR (400 MHz, CDCl₃) δ 8.09-8.03 (m, 1H), 7.05 (d, J=8.4 Hz, 1H), 6.91 (d, J=2.4 Hz, 1H), 6.78 (dd, J=2.4, 8.4 Hz, 1H), 4.33 (s, 2H), 4.08 (dd, J=5.2, 10.8 Hz, 1H), 3.95 (d, J=14.4 Hz, 4H), 3.40 (s, 3H), 2.75 (t, J=4.4 Hz, 1H), 2.70 (dd, J=5.4, 11.2 Hz, 1H), 2.10 (d, J=10.0 Hz, 3H), 2.05-2.03 (m, 1H), 2.01 (d, J=4.0 Hz, 1H), 1.83 (t, J=11.2 Hz, 4H), 1.75 (d, J=12.0 Hz, 1H), 1.62-1.55 (m, 2H), 1.43 (s, 9H), 1.26 (s, 3H); LC-MS (ESI⁺) m/z 561.1 (M+H)⁺.

### Step 2—3-[4-[(3R)-4-[(4-amino-2-oxabicyclo[2.2.2]octan-1-yl)methyl]-3-methyl-piperazin-1-yl]-2-chloro-phenyl]piperidine-2,6-dione

To a solution of tert-butyl N-[1-[[(2R)-4-[3-chloro-4-(2,6-dioxo-3-piperidyl)phenyl]-2-methyl-piperazin-1-yl]methyl]-2-oxabicyclo[2.2.2]octan-4-yl]carbamate (80 mg, 142 μmol) in DCM (0.5 mL) was added HCl/dioxane (4 M, 2 mL), then the mixture was stirred at 25° C. for 0.5 hr. On completion, the reaction mixture was concentrated in vacuo to give the title compound (70.9 mg, 99% yield, HCl) as yellow oil. LC-MS (ESI⁺) m/z 461.0 (M+H)⁺.

### Synthesis of 4-Benzylsulfanyl-2-fluoro-aniline (Intermediate PU) and 2-benzylsulfanyl-4-fluoro-aniline (Intermediate PV)

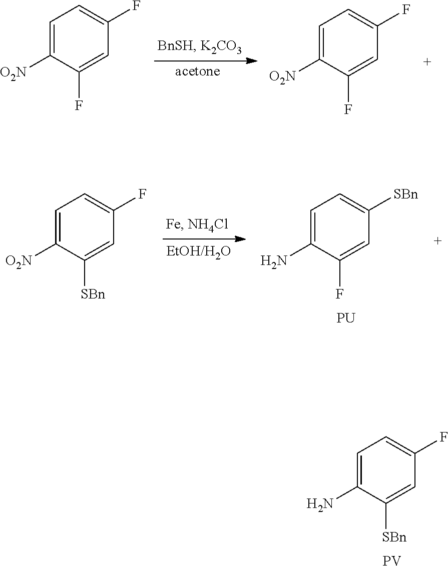

### Step 1—4-Benzylsulfanyl-2-fluoro-1-nitro-benzene and 2-Benzylsulfanyl-4-fluoro-1-nitro-benzene

To a solution of 2,4-difluoro-1-nitro-benzene (10.0 g, 62.8 mmol, CAS #446-35-5) in acetone (100 mL) was added K₂CO₃ (17.3 g, 125 mmol), then BnSH (6.21 g, 50.0 mmol, CAS #100-53-8) was added and the mixture was stirred at 56° C. for 12 hrs. On completion, the mixture was poured into ice water (100 mL), and extracted with DCM (100 mL×3). The combined organic layer was dried over anhydrous Na₂SO₄, filtered and concentrated in vacuo to give the residue. The water phase was quenched with NaClO (80 mL), then discarded. The residue was purified by reverse-phase (0.1% FA condition) to give the title compounds (9.2 g, 55% yield, mixture of regioisomers) as yellow solid. ¹H NMR (400 MHz, DMSO-d₆) δ 8.32 (dd, J=5.6, 9.2 Hz, 1H), 7.63-7.50 (m, 2H), 7.44 (s, 1H), 7.40-7.35 (m, 2H), 7.31-7.21 (m, 2H), 4.40 (s, 2H).

### Step 2—4-Benzylsulfanyl-2-fluoro-aniline & 2-benzylsulfanyl-4-fluoro-aniline

To a solution of 4-benzylsulfanyl-2-fluoro-1-nitro-benzene (9.20 g, 34.9 mmol, mixture of regioisomers) in EtOH (100 mL) and H₂O (20 mL) was added Fe (11.7 g, 209 mmol) and NH₄Cl (18.6 g, 349 mmol), then the mixture was stirred at 80° C. for 2 hrs. On completion, the mixture was filtered and concentrated in vacuo to give the residue. SFC indicated two peaks, the first peak was 2-benzylsulfanyl-4-fluoro-aniline and the second peak was 4-benzylsulfanyl-2-fluoro-aniline, which were confirmed by 2D NMR. The residue was purified by column chromatography (SiO₂, PE:EA=1:1), which was further separated by SFC (column: DAICEL CHIRALCEL OJ (250 mm*30 mm, 10 um); mobile phase: [CO₂-MeOH]; B %: 35%, isocratic elution mode) to give 4-benzylsulfanyl-2-fluoro-aniline (2.40 g, 29% yield) as gray oil (¹H NMR (400 MHz, DMSO-d₆) δ 7.29-7.24 (m, 2H), 7.23-7.17 (m, 3H), 6.97 (dd, J=2.0, 11.6 Hz, 1H), 6.86 (dd, J=1.6, 8.4 Hz, 1H), 6.66 (dd, J=8.4, 9.6 Hz, 1H), 5.27 (s, 2H), 4.01 (s, 2H). LC-MS (ESI⁺) m/z 234.1 (M+H)+) and 2-benzylsulfanyl-4-fluoro-aniline (4.2 g, 51.52% yield) as gray oil (¹H NMR (400 MHz, DMSO-d₆) δ 7.30-7.18 (m, 5H), 6.91 (dd, J=2.8, 9.2 Hz, 1H), 6.87-6.82 (m, 1H), 6.70 (dd, J=5.2, 8.8 Hz, 1H), 5.11 (s, 2H), 4.04 (s, 2H). LC-MS (ESI⁺) m/z 233.9 (M+H)+).

### Synthesis of 3-Fluoro-4-[[4-[(3S)-3-hydroxy-3-methyl-1-piperidyl]-5-(trifluoromethyl)pyrimidin-2-yl] amino]benzenesulfonyl chloride (Intermediate PW)

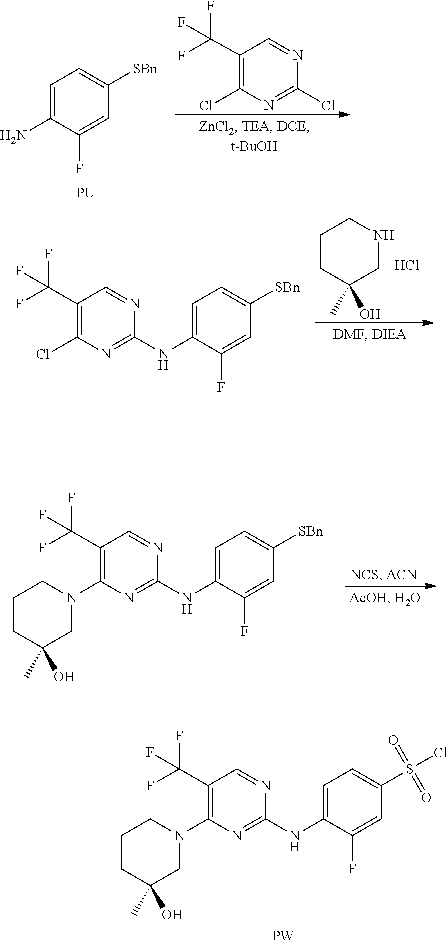

### Step 1—N-(4-benzylsulfanyl-2-fluoro-phenyl)-4-chloro-5-(trifluoromethyl)pyrimidin-2-amine

To a solution of 2,4-dichloro-5-(trifluoromethyl)pyrimidine (372 mg, 1.71 mmol, CAS #3932-97-6) in t-BuOH (4 mL) and DCE (4 mL) was added ZnCl₂ (1 M, 2.06 mL) at 0° C. and the mixture was stirred at 0° C. for 1 hr. Then 4-benzylsulfanyl-2-fluoro-aniline (400 mg, 1.71 mmol, Intermediate PU) and TEA (190 mg, 1.89 mmol) in DCE (4 mL) and t-BuOH (4 mL) was added at 0° C., then the mixture was stirred at 25° C. for 14 hrs. On completion, the mixture was diluted with water (5 mL) and extracted with EA (5 mL×3). The combined organic layer was dried over anhydrous Na₂SO₄, filtered and concentrated in vacuo to give the residue. The residue was purified by prep-HPLC (column: Phenomenex luna C18 150*25 mm*10 um; mobile phase: [water (FA)-ACN]; gradient: 69%-99% B over 10 min) to give the title compound (300 mg, 42% yield) as yellow solid. ¹H NMR (400 MHz, DMSO-d₆) δ 10.32 (s, 1H), 8.73 (s, 1H), 7.45 (t, J=8.4 Hz, 1H), 7.41-7.37 (m, 2H), 7.34-7.29 (m, 3H), 7.27-7.22 (m, 1H), 7.17 (dd, J=1.6, 8.4 Hz, 1H), 4.31 (s, 2H). LC-MS (ESI⁺) m/z 413.9 (M+H)⁺.

### Step 2—(3S)-1-[2-(4-benzylsulfanyl-2-fluoro-anilino)-5-(trifluoromethyl)pyrimidin-4-yl]-3-methyl-piperidin-3-ol

To a solution of N-(4-benzylsulfanyl-2-fluoro-phenyl)-4-chloro-5-(trifluoromethyl)pyrimidin-2-amine (100 mg, 241 μmol) in DMF (1 mL) was added DIEA (31.2 mg, 241 μmol), then (3S)-3-methylpiperidin-3-ol (54.9 mg, 362 μmol, CAS #2305080-37-7) was added and the mixture was stirred at 25° C. for 2 hrs. On completion, the mixture was filtered to give the residue. The residue was purified by prep-HPLC (column: Phenomenex luna C18 150*25 mm*10 um; mobile phase: [water (FA)-ACN]; gradient: 61%-91% B over 10 min) to give the title compound (90.0 mg, 75% yield) as white solid. ¹H NMR (400 MHz, CDCl₃) δ 8.35 (s, 1H), 8.14 (t, J=8.4 Hz, 1H), 7.32-7.27 (m, 2H), 7.27-7.20 (m, 4H), 7.12-7.03 (m, 2H), 4.07 (s, 2H), 4.00 (d, J=13.6 Hz, 1H), 3.89-3.86 (m, 1H), 3.18-3.09 (m, 1H), 3.06 (d, J=13.6 Hz, 1H), 1.98-1.84 (m, 1H), 1.80-1.77 (m, 1H), 1.68-1.57 (m, 2H), 1.56-1.50 (m, 1H), 1.24 (s, 3H). LC-MS (ESI⁺) m/z 493.3 (M+H)⁺.

### Step 3—3-fluoro-4-[[4-[(3S)-3-hydroxy-3-methyl-1-piperidyl]-5-(trifluoromethyl)pyrimidin-2-yl]amino]benzenesulfonyl chloride

To a solution of (3S)-1-[2-(4-benzylsulfanyl-2-fluoro-anilino)-5-(trifluoromethyl)pyrimidin-4-yl]-3-methyl-piperidin-3-ol (69.0 mg, 140 μmol) in ACN (1 mL), AcOH (0.1 mL) and H₂O (0.01 mL) was added NCS (74.8 mg, 560 μmol). Then the mixture was stirred at 25° C. for 40 mins under dark atmosphere. On completion, the mixture was diluted with water (3 mL) and extracted with EA (3 mL×3). The combined organic layer was dried over anhydrous Na₂SO₄, filtered and concentrated in vacuo to give the title compound (50.0 mg, 76% yield) as colorless oil. LC-MS (ESI⁺) m/z 468.9 (M+H)⁺.

### Synthesis of 6-Chloro-8-(1-cyclopropylethyl)-2-methylsulfonyl-pyrido[2,3-d]pyrimidin-7-one (Intermediate PX)

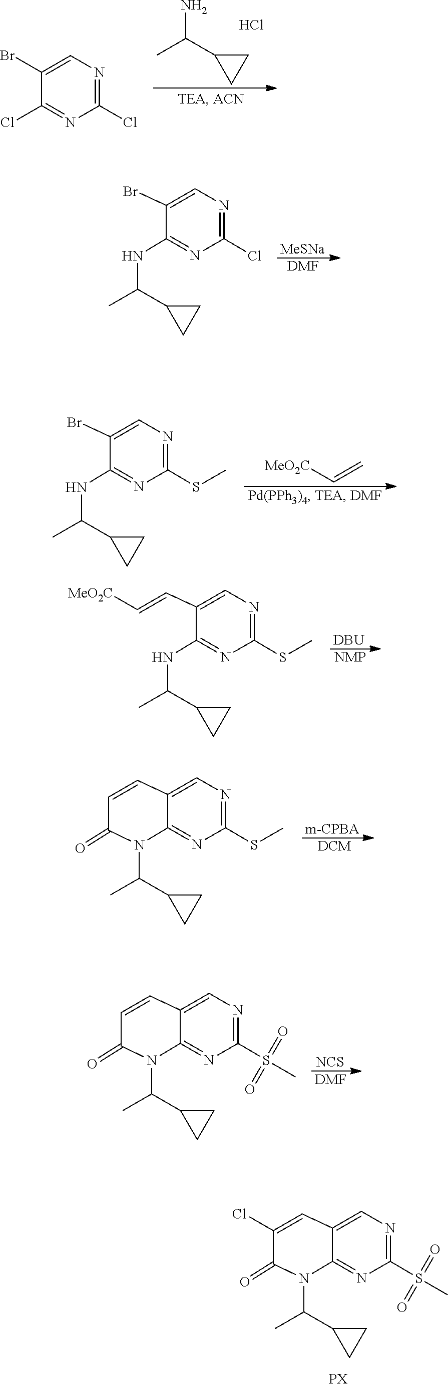

### Step 1—5-Bromo-2-chloro-N-(1-cyclopropylethyl)pyrimidin-4-amine

To a solution of 5-bromo-2,4-dichloro-pyrimidine (20.0 g, 87.7 mmol, 11.2 mL, CAS #36082-50-5) in MeCN (200 mL) was added TEA (20.4 g, 201 mmol, 28.1 mL) and 1-cyclopropylethanamine hydrochloride (13.9 g, 114 mmol, CAS #42390-64-7) at 0° C. Then the mixture was stirred at 25° C. for 16 hrs. On completion, the mixture was poured into H₂O (200 mL), and extracted with EA (200 mL×3). The organic layer was washed with brine (100 mL) and dried with anhydrous Na₂SO₄, filtered and concentrated in vacuo to give the title compound (23.4 g, 91% yield) as white solid. LC-MS (ESI⁺) m/z 277.8 (M+3)+.

### Step 2—5-Bromo-N-(1-cyclopropylethyl)-2-methylsulfanyl-pyrimidin-4-amine

To a solution of 5-bromo-2-chloro-N-(1-cyclopropylethyl)pyrimidin-4-amine (23.4 g, 84.6 mmol) in DMF (230 mL) was added NaSMe (14.8 g, 211 mmol, 13.5 mL) at 0° C., then the mixture was stirred at 25° C. for 12 hours. On completion, the mixture was poured into H₂O (300 mL) and extracted with EA (300 mL×3). Then the combined organic layer was dried over anhydrous Na₂SO₄, filtered and concentrated in vacuo to give the title compound (24.0 g, 98% yield) as yellow solid. LC-MS (ESI⁺) m/z 290.0 (M+3)+. The water layer was quenched with NaClO (100 mL) at 0° C., then discarded.

### Step 3—Methyl (E)-3-[4-(1-cyclopropylethylamino)-2-methylsulfanyl-pyrimidin-5-yl]prop-2-enoate

To a solution of 5-bromo-N-(1-cyclopropylethyl)-2-methylsulfanyl-pyrimidin-4-amine (12 g, 41.6 mmol) in DMF (150 mL) was added Pd(PPh₃)₄ (4.81 g, 4.16 mmol) and TEA (12.6 g, 125 mmol, 17.4 mL), then methyl prop-2-enoate (31.8 g, 370 mmol, 33.3 mL) was added at 25 C. The mixture was then stirred at 90° C. for 48 hrs under N₂ atmosphere. On completion, the mixture was quenched with NaClO (50 mL) at 0° C., diluted with H₂O (300 mL), and extracted with EA (300 mL×3). The combined organic layer was washed with saturated brine (100 mL), dried over anhydrous Na₂SO₄, filtered and concentrated in vacuo to give the residue. The residue was purified by column chromatography (SiO₂, Petroleum ether/Ethyl acetate=20:1 to 4:1) to give the title compound (4.20 g, 34% yield) as a yellow oil. ¹H NMR (400 MHz, DMSO-d₆) δ 8.36 (s, 1H), 7.81 (d, J=16.0 Hz, 1H), 7.65 (d, J=8.0 Hz, 1H), 6.51 (d, J=15.6 Hz, 1H), 3.72 (s, 3H), 3.70-3.64 (m, 1H), 2.41 (s, 3H), 1.24-1.22 (m, 3H), 1.11-1.03 (m, 1H), 0.49-0.36 (m, 2H), 0.31-0.18 (m, 2H). LC-MS (ESI⁺) m/z 294.4 (M+H)⁺.

### Step 4—8-(1-Cyclopropylethyl)-2-methylsulfanyl-pyrido[2,3-d]pyrimidin-7-one

To a solution of methyl (E)-3-[4-(1-cyclopropylethylamino)-2-methylsulfanyl-pyrimidin-5-yl]prop-2-enoate (3.00 g, 10.2 mmol) in NMP (30 mL) was added DBU (7.78 g, 51.1 mmol, 7.71 mL), then the mixture was stirred at 120° C. for 2 hrs. On completion, the reaction mixture was diluted with H₂O (100 mL) and extracted with DCM (50 mL×3). The combined organic layer was dried over anhydrous Na₂SO₄, filtered and concentrated in vacuo to give the title compound (3.20 g, 59% yield) as a brown oil. ¹H NMR (400 MHz, DMSO-d₆) δ 8.87 (s, 1H), 7.91 (d, J=9.6 Hz, 1H), 6.60 (s, 1H), 4.86-4.46 (m, 1H), 2.52 (s, 3H), 2.05-2.02 (m, 1H), 1.68-1.56 (m, 3H), 0.66-0.58 (m, 1H), 0.38 (s, 2H), 0.08 (s, 1H). LC-MS (ESI⁺) m/z 262.0 (M+H)⁺.

### Step 5—8-(1-Cyclopropylethyl)-2-methylsulfonyl-pyrido[2,3-d]pyrimidin-7-one

To a solution of 8-(1-cyclopropylethyl)-2-methylsulfanyl-pyrido[2,3-d]pyrimidin-7-one (2.00 g, 7.65 mmol) in DCM (20 mL) was added m-CPBA (3.42 g, 16.8 mmol, 85% solution) at 0° C., then the mixture was stirred at 25° C. for 1 hr. On completion, the reaction mixture was quenched with saturated Na₂SO₃ (10 mL) and saturated Na₂CO₃ (10 mL) at 0° C., diluted with H₂O (30 mL) and extracted with EA (20 mL×3). The combined organic layer was dried over anhydrous Na₂SO₄, filtered and concentrated in vacuo to give a residue. The residue was purified by reversed-phase (0.1% FA condition) to give the title compound (1.00 g, 44% yield) as a white solid. ¹H NMR (400 MHz, DMSO-d₆) δ 9.29 (s, 1H), 8.10 (d, J=9.6 Hz, 1H), 6.90 (d, J=9.2 Hz, 1H), 4.80-4.47 (m, 1H), 3.45 (s, 3H), 2.09-1.86 (m, 1H), 1.65 (s, 3H), 0.64-0.63 (m, 1H), 0.46-0.27 (m, 2H), 0.11 (s, 1H). LC-MS (ESI⁺) m/z 294.1 (M+H)⁺.

### Step 6—6-Chloro-8-(1-cyclopropylethyl)-2-methylsulfonyl-pyrido[2,3-d]pyrimidin-7-one

To a solution of 8-(1-cyclopropylethyl)-2-methylsulfonyl-pyrido[2,3-d]pyrimidin-7-one (300 mg, 1.02 mmol) in DMF (5 mL) was added NCS (409 mg, 3.07 mmol), then the mixture was stirred at 70° C. for 48 hours. On completion, the reaction mixture was diluted with H₂O (20 mL) and extracted with EA (10 mL×3). The combined organic layer was dried over anhydrous Na₂SO₄, filtered and concentrated in vacuo to give a residue. Then the residue was purified by column chromatography (SiO₂, Petroleum ether/Ethyl acetate=10:1 to 2:1) to give the title compound (1.20 g, 89% yield) as a yellow oil. ¹H NMR (400 MHz, DMSO-d₆) δ 9.27 (s, 1H), 8.53 (s, 1H), 4.88-4.51 (m, 1H), 3.46 (s, 3H), 2.49-2.37 (m, 1H), 1.67 (s, 3H), 0.65-0.64 (m, 1H), 0.48-0.43 (m, 1H), 0.35-0.30 (m, 1H), 0.15 (s, 1H). LC-MS (ESI⁺) m/z 328.1 (M+H)⁺.

### Synthesis of 2-[4-(7-azaspiro[3.5]nonan-2-ylsulfonyl)-2-methyl-anilino]-6-chloro-8-(1-cyclopropylethyl)pyrido[2,3-d]pyrimidin-7-one (Intermediate PY)

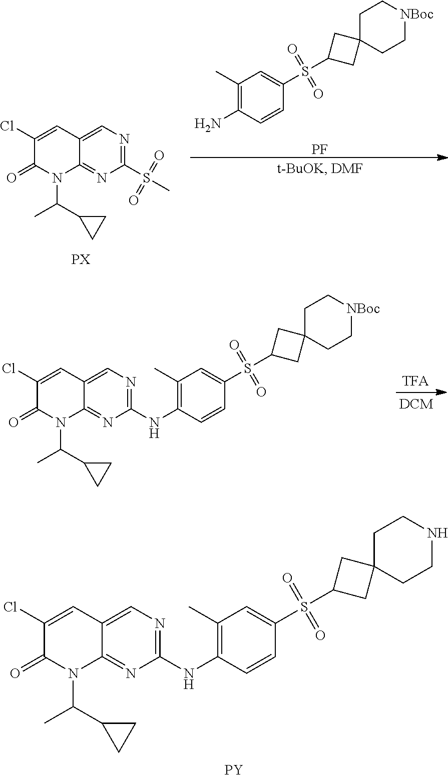 [SMILES: Cc1cc(S(=O)(=O)C2CC3(CCN(C(=O)OC(C)(C)C)CC3)C2)ccc1N ; CC(C1CC1)n1c(=O)c(Cl)cc2cnc(S(C)(=O)=O)nc21 ; "PF" ; Cc1cc(S(=O)(=O)C2CC3(CCN(C(=O)OC(C)(C)C)CC3)C2)ccc1Nc1ncc2cc(Cl)c(=O)n(C(C)C3CC3)c2n1 ; Cc1cc(S(=O)(=O)C2CC3(CCNCC3)C2)ccc1Nc1ncc2cc(Cl)c(=O)n(C(C)C3CC3)c2n1 ; "PY"]

### Step 1—Tert-butyl 2-[4-[[6-chloro-8-(1-cyclopropylethyl)-7-oxo-pyrido[2,3-d]pyrimidin-2-yl]amino]-3-methyl-phenyl]sulfonyl-7-azaspiro[3.5]nonane-7-carboxylate

To a solution of tert-butyl 2-(4-amino-3-methyl-phenyl)sulfonyl-7-azaspiro[3.5]nonane-7-carboxylate (320 mg, 811 mol, Intermediate PF) in DMF (10 mL), was added t-BuOK (273 mg, 2.43 mmol) was at 0° C., then 6-chloro-8-(1-cyclopropylethyl)-2-methylsulfonyl-pyrido[2,3-d]pyrimidin-7-one (797.60 mg, 2.43 mmol, Intermediate PX). Finally, the mixture was stirred at 25° C. for 2 hrs under N₂ atmosphere. On completion, the reaction mixture was diluted with H₂O (20 mL) and extracted with EA (10 mL×3). The combined organic layer was dried over anhydrous Na₂SO₄, filtered and concentrated in vacuo to give a residue. Then the residue was purified by column chromatography (SiO₂, Petroleum ether/Ethyl acetate=8:1 to 2:1) to give the title compound (100 mg, 19% yield) as a yellow solid. ¹H NMR (400 MHz, DMSO-d₆) δ 9.76-9.66 (m, 1H), 8.77 (s, 1H), 8.24-8.17 (m, 1H), 7.80-7.65 (m, 3H), 4.13 (t, J=8.4 Hz, 1H), 2.89 (s, 3H), 2.73 (s, 3H), 2.36-2.32 (m, 4H), 2.13-2.05 (m, 4H), 2.01-1.92 (m, 4H), 1.52-1.41 (m, 9H), 1.23 (s, 1H), 0.89-0.78 (m, 1H), 0.37-0.19 (m, 2H), 0.17--0.01 (m, 2H). LC-MS (ESI⁺) m/z 642.2 (M+H)⁺.

### Step 2—2-[4-(7-azaspiro[3.5]nonan-2-ylsulfonyl)-2-methyl-anilino]-6-chloro-8-(1-cyclopropylethyl)pyrido[2,3-d]pyrimidin-7-one

To a solution of tert-butyl 2-[4-[[6-chloro-8-(1-cyclopropylethyl)-7-oxo-pyrido[2,3-d]pyrimidin-2-yl]amino]-3-methyl-phenyl]sulfonyl-7-azaspiro[3.5]nonane-7-carboxylate (50.0 mg, 77.8 μmol) in DCM (2 mL) was added TFA (8.88 mg, 77.8 μmol, 5.78 μL), then the mixture was stirred at 25° C. for 0.5 hr. On completion, the reaction mixture was concentrated in vacuo to give the title compound (40.0 mg, 78% yield, TFA) as a brown oil. LC-MS (ESI⁺) m/z 542.2 (M+H)⁺.

### Synthesis of 8-Cyclopentyl-2-(methylsulfonyl)-5,8-dihydropyrido[2,3-d]pyrimidin-7(6H)-one (Intermediate PZ)

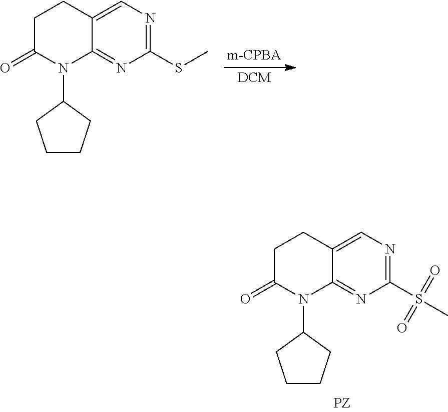 [SMILES: CSc1ncc2c(n1)N(C1CCCC1)C(=O)CC2 ; CS(=O)(=O)c1ncc2c(n1)N(C1CCCC1)C(=O)CC2]

To a solution of 8-cyclopentyl-2-(methylthio)-5,8-dihydropyrido[2,3-d]pyrimidin-7(6H)-one (260 mg, 987 mol, Intermediate HN) in DCM (8 mL) was added m-CPBA (801 mg, 3.95 mmol, 85% solution). The mixture was stirred at 25° C. for 1 hr. The reaction mixture was diluted with Na₂SO₄ (20 mL) and extracted with DCM (20 mL×3). The combined organic layers were washed with NaHCO₃ (20 mL) and NaCl (20 mL), dried over Na₂SO₄, filtered and concentrated in vacuo to give the title compound (253 mg, 87% yield) as white oil. LC-MS (ESI⁺) m/z 296.0 (M+H)⁺.

### Synthesis of 2-((4-((7-Azaspiro[3.5]nonan-2-yl)sulfonyl)-2-methylphenyl)amino)-8-cyclopentyl-5,8-dihydropyrido[2,3-d]pyrimidin-7(6H)-one (Intermediate QA)

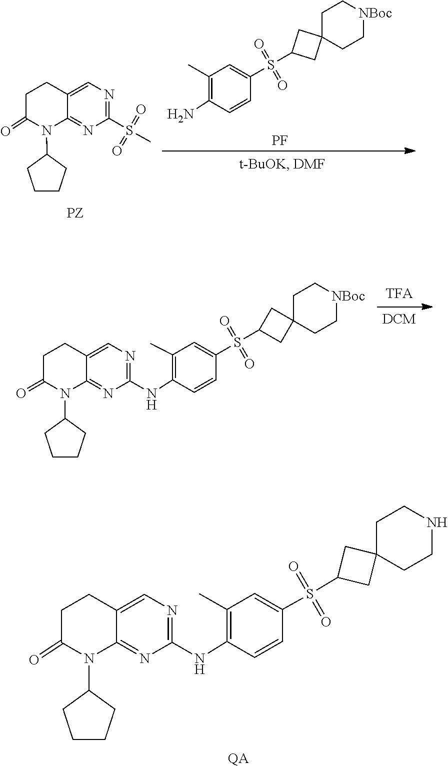

### Step 1—Tert-butyl 2-((4-((8-cyclopentyl-7-oxo-5,6,7,8-tetrahydropyrido[2,3-d]pyrimidin-2-yl)amino)-3-methylphenyl)sulfonyl)-7-azaspiro[3.5]nonane-7-carboxylate

A mixture of 8-cyclopentyl-2-(methylsulfonyl)-5,8-dihydropyrido[2,3-d]pyrimidin-7(6H)-one (329 mg, 1.11 mmol, Intermediate PZ), tert-butyl 2-((4-amino-3-methylphenyl)sulfonyl)-7-azaspiro[3.5]nonane-7-carboxylate (439 mg, 1.11 mmol, Intermediate PF), and t-BuOK (499 mg, 4.46 mmol) at 0° C. in DMF (2 mL) was degassed and purged with N₂ three times, and then the mixture was stirred at 0° C. for 1 hr under N₂ atmosphere. On completion, the reaction mixture was concentrated in vacuo to give a residue. The residue was firstly purified by prep-HPLC (column: Phenomenex luna C18 150*25 mm*10 um; mobile phase: [water(FA)-ACN]; B %: 53%-83%, 10 min), then was purified by prep-HPLC (column: Phenomenex luna C18 150*25 mm*10 um; mobile phase: [water(FA)-ACN]; B %: 58%-88%, 10 min) to give the title compound (30.0 mg, 4% yield) as a yellow solid. LC-MS (ESI⁺) m z 610.4 (M+H)⁺.

### Step 2—1-(1-(2,6-Dioxopiperidin-3-yl)-4-fluoro-3-methyl-2-oxo-2,3-dihydro-1H-benzo[d]imidazol-5-yl)piperidine-4-carbaldehyde

To a solution of 3-[5-[4-(dimethoxymethyl)-1-piperidyl]-4-fluoro-3-methyl-2-oxo-benzimidazol-1-yl]piperidine-2,6-dione (30.0 mg, 69.0 μmol) was added HCOOH (3.32 mg, 69.05 μmol, 1 mL). The mixture was then stirred at 80° C. for 1 hr. On completion, the reaction mixture was concentrated in vacuo to give the title compound (24.0 mg, 89% yield) as a brown solid. LC-MS (ESI⁺) m/z 389.0 (M+H)⁺.

### Step 3-2-((4-((7-Azaspiro[3.5]nonan-2-yl)sulfonyl)-2-methylphenyl)amino)-8-cyclopentyl-5,8-dihydropyrido[2,3-d]pyrimidin-7(6H)-one

To a solution of tert-butyl 2-((4-((8-cyclopentyl-7-oxo-5,6,7,8-tetrahydropyrido[2,3-d]pyrimidin-2-yl)amino)-3-methylphenyl)sulfonyl)-7-azaspiro[3.5]nonane-7-carboxylate (30.0 mg, 49.2 μmol,) in DCM (1.5 mL) was added TFA (767 mg, 6.73 mmol, 0.5 mL). The mixture was then stirred at 25° C. for 0.5 hr. On completion, the reaction mixture was concentrated in vacuo to give the title compound (25.0 mg, 99% yield) as a yellow solid. LC-MS (ESI⁺) m/z 510.2 (M+H)⁺.

### Synthesis of 1-[2-[4-(7-Azaspiro[3.5]nonan-2-ylsulfonyl)-2-methyl-anilino]-5-(trifluoromethyl)pyrimidin-4-yl]-4-methyl-piperidin-4-ol (Intermediate QB)

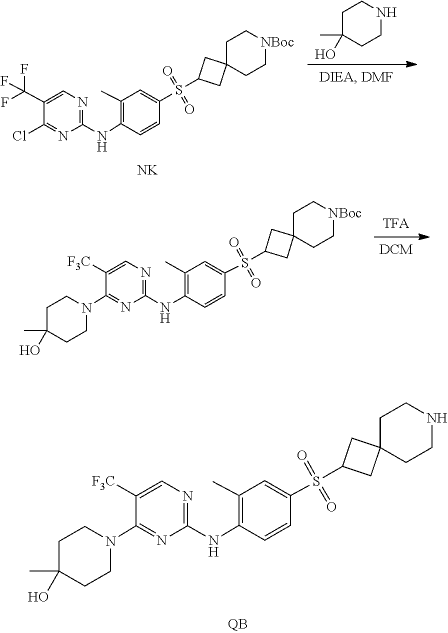

### Step 1—Tert-butyl 2-[4-[[4-(4-hydroxy-4-methyl-1-piperidyl)-5-(trifluoromethyl) pyrimidin-2-yl] amino]-3-methyl-phenyl]sulfonyl-7-azaspiro[3.5]nonane-7-carboxylate

To a solution of 4-methylpiperidin-4-ol (48.0 mg, 417 μmol, CAS #3970-68-1) in ACN (4 mL) was added TEA (363 mg, 0.5 mL), then tert-butyl 2-[4-[[4-chloro-5-(trifluoromethyl) pyrimidin-2-yl] amino]-3-methyl-phenyl] sulfonyl-7-azaspiro[3.5]nonane-7-carboxylate (200 mg, 347 μmol, Intermediate NK) was added to the former mixture. Then the mixture was stirred at 25° C. for 2 hrs. On completion, the mixture was diluted with EA (10 mL) and water (20 mL), then extracted with EA (3×10 mL). The combined organic layer was dried over anhydrous Na₂SO₄, filtered and concentrated in vacuo to give the title compound (200 mg, 88% yield) as a white solid. ¹H NMR (400 MHz, DMSO-d₆) δ 1.14 (s, 3H), 1.17 (m, 2H), 1.37 (s, 9H), 1.39-1.43 (m, 2H), 1.48-1.52 (m, 4H), 1.92-2.00 (m, 3H), 2.04-2.12 (m, 2H), 2.34 (s, 3H), 3.1-3.25 (m, 4H), 3.36-3.40 (m, 1H), 3.72 (d, J=13.2 Hz, 2H), 4.02-4.11 (m, 1H), 4.42 (s, 1H), 7.5-7.72 (m, 2H), 7.92 (d, J=8.4 Hz, 1H), 8.37 (s, 1H), 9.14 (s, 1H). LC-MS (ESI⁺) m/z 654.4 (M+H)⁺.

### Step 2—1-[2-[4-(7-Azaspiro[3.5]nonan-2-ylsulfonyl)-2-methyl-anilino]-5-(trifluoromethyl)pyrimidin-4-yl]-4-methyl-piperidin-4-ol

To a solution of tert-butyl 2-[4-[[4-(4-hydroxy-4-methyl-1-piperidyl)-5-(trifluoromethyl)pyrimidin-2-yl]amino]-3-methyl-phenyl]sulfonyl-7-azaspiro[3.5]nonane-7-carboxylate (100 mg, 152 mol) in DCM (4 mL) was added TFA (16.8 mmol, 1.25 mL), then the mixture was stirred at 25° C. for 1 hr. On completion, the mixture was concentrated in vacuo to give the compound (100 mg, 97% yield, TFA) as yellow solid. LC-MS (ESI⁺) m/z 554.2 (M+H)⁺.

### Synthesis of 1-[1-(2,6-Dioxo-3-piperidyl)-2-oxo-4-pyridyl]piperidine-4-carbaldehyde (Intermediate QC)

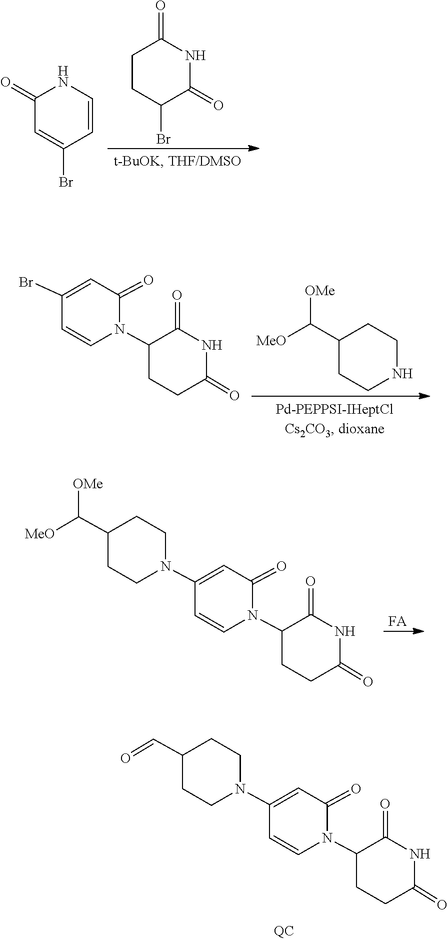

### Step 1—3-(4-Bromo-2-oxopyridin-1(2H)-yl)piperidine-2,6-dione

To a mixture of 4-bromopyridin-2(1H)-one (10.0 g, 57.5 mmol, CAS #36953-37-4) in THF (50.0 mL) and DMSO (50.0 mL) was added t-BuOK (12.9 g, 115 mmol) at 0° C. and the mixture was stirred for 0.5 h. Then 3-bromopiperidine-2,6-dione (16.6 g, 86.2 mmol, CAS #62595-74-8) in THF (50.0 mL) was added at 0° C. and the resulting mixture was allowed to warm up to rt and the mixture was stirred for 16 h. On completion, the reaction mixture was poured into saturated aqueous NH₄Cl (1.0 L), extracted with ethyl acetate (200 mL×3) and MeCN (200 mL×3). The combined organic layers were dried over anhydrous Na₂SO4, filtered and concentrated under reduced pressure to give a residue (16.0 g). The residue (16.0 g) was triturated with ethyl acetate (300 mL) at rt for 1 h, filtered to give a filter cake as the crude product (3.8 g). The crude was further triturated with MeOH (30.0 mL) at rt for 1 h, filtered and the filter cake was dried to give the title compound (2.06 g, 12% yield) as a purple solid. LC-MS (ESI⁺) m/z 287.2 & 285.2 (⁸¹Br+H & ⁷⁹Br+H)⁺; ¹H NMR (400 MHz, DMSO-d₆) δ=11.06 (s, 1H), 7.65 (d, J=7.2 Hz, 1H), 6.78 (d, J=2.0 Hz, 1H), 6.54 (dd, J=2.0, 7.2 Hz, 1H), 5.64-5.13 (m, 1H), 2.93-2.70 (m, 1H), 2.66-2.52 (m, 2H), 2.07-1.91 (m, 1H).

### Step 2—3-[4-[4-(Dimethoxymethyl)-1-piperidyl]-2-oxo-1-pyridyl]piperidine-2,6-dione

To a solution of 3-(4-bromo-2-oxo-1-pyridyl)piperidine-2,6-dione (110 mg, 386 μmol) and 4-(dimethoxy methyl)piperidine (92.2 mg, 579 μmol, CAS #188646-83-5) in dioxane (1 mL) was added Cs₂CO₃ (377 mg, 1.16 mmol), and Pd-PEPPSI-IHeptCl (37.5 mg, 38.6 μmol). The mixture was then stirred at 100° C. for 16 hrs. On completion, the mixture was filtered, and the filtrate was diluted with H₂O (5 mL) and DCM (5 mL), then extracted with DCM (10 mL×3). The combined organic layer was dried over Na₂SO₄, then filtered and concentrated in vacuo to give the residue. The residue was purified by prep-HPLC (column: Phenomenex luna C18 150*25 mm*10 um; mobile phase: [water (FA)-ACN]; gradient: 10%-40% B over 8 min) to give the title compound (10.0 mg, 7% yield) as a white solid. ¹H NMR (400 MHz, CHLOROFORM-d) δ 7.96 (s, 1H), 6.99-6.91 (m, 1H), 6.03-6.00 (m, 1H), 5.77-5.68 (m, 1H), 3.84 (d, J=12.4 Hz, 2H), 2.86 (s, 3H), 2.58-2.45 (m, 1H), 2.26-2.21 (m, 1H), 1.82 (d, J=14.4 Hz, 2H), 1.40-1.24 (m, 5H). LC-MS (ESI⁺) m/z 364.1 (M+H)⁺.

### Step 3—1-[1-(2,6-Dioxo-3-piperidyl)-2-oxo-4-pyridyl]piperidine-4-carbaldehyde

To a solution of 3-[4-[4-(dimethoxymethyl)-1-piperidyl]-2-oxo-1-pyridyl]piperidine-2,6-dione (10.0 mg, 27.5 μmol) in formic acid (1 mL) was stirred at 60° C. for 0.5 hr. On completion, the mixture was concentrated in vacuo to give the title compound (8.20 mg, 94% yield) as a white solid. LC-MS (ESI⁺) m z 363.2 (M+H)⁺.

### Synthesis of 2-[1-[2-[4-(7-Azaspiro[3.5]nonan-2-ylsulfonyl)-2-methyl-anilino]-5-(trifluoromethyl) pyrimidin-4-yl]pyrrolidin-3-yl]propan-2-ol (Intermediate QD)

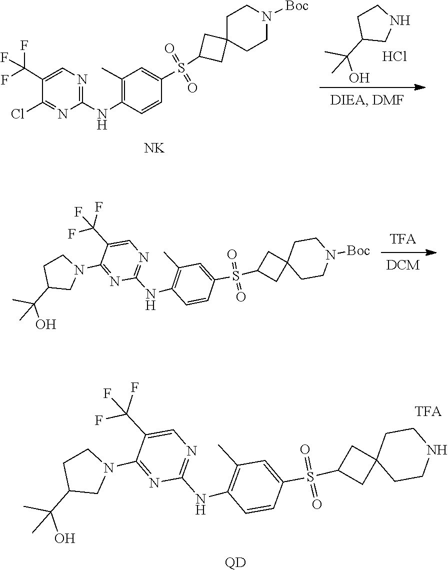

### Step 1—Tert-butyl 2-[4-[[4-[3-(1-hydroxy-1-methyl-ethyl)pyrrolidin-1-yl]-5-(trifluoromethyl)pyrimidin-2-yl]amino]-3-methyl-phenyl]sulfonyl-7-azaspiro[3.5]nonane-7-carboxylate

To a solution of 2-pyrrolidin-3-ylpropan-2-ol; hydrochloride (34.6 mg, 208 μmol, CAS #1357923-37-5) in ACN (2 mL) was added TEA (48.4 μL, 348 μmol). Then tert-butyl 2-[4-[[4-chloro-5-(trifluoromethyl)pyrimidin-2-yl]amino]-3-methyl-phenyl]sulfonyl-7-azaspiro[3.5]nonane-7-carboxylate (100 mg, 174 mol, Intermediate NK) was added and the mixture was stirred at 25° C. for 2 hrs. On completion, the mixture was diluted with water (50 mL) and extracted with EA (20 mL×3). The combined organic layers were dried over anhydrous Na₂SO₄, filtered and concentrated in vacuo to give the title compound (100 mg, 86% yield) as yellow solid. ¹H NMR (400 MHz, DMSO-d₆) δ 8.97 (s, 1H), 8.33 (s, 1H), 8.06 (d, J=8.4 Hz, 1H), 7.66 (s, 1H), 7.61-7.59 (m, 1H), 4.42 (s, 1H), 4.10-4.03 (m, 1H), 3.72-3.63 (m, 1H), 3.56-3.54 (m, 1H), 3.51-3.42 (m, 2H), 3.24-3.15 (m, 4H), 2.35 (s, 3H), 2.19-2.17 (m, 1H), 2.07-2.05 (m, 2H), 1.98-1.92 (m, 2H), 1.90-1.89 (m, 1H), 1.81-1.76 (m, 1H), 1.51-1.46 (m, 2H), 1.44-1.39 (m, 2H), 1.37 (s, 9H), 1.11 (d, J=4.4 Hz, 6H). LC-MS (ESI⁺) m/z 668.3 (M+H)⁺.

### Step 2—2-[1-[2-[4-(7-Azaspiro[3.5]nonan-2-ylsulfonyl)-2-methyl-anilino]-5-(trifluoromethyl) pyrimidin-4-yl]pyrrolidin-3-yl]propan-2-ol

A solution of tert-butyl 2-[4-[[4-[3-(1-hydroxy-1-methyl-ethyl)pyrrolidin-1-yl]-5-(trifluoromethyl) pyrimidin-2-yl]amino]-3-methyl-phenyl]sulfonyl-7-azaspiro[3.5]nonane-7-carboxylate (75.0 mg, 112 mol) in DCM (1 mL) and TFA (0.3 mL) was stirred at 25° C. for 1 hr. On completion, the mixture was concentrated in vacuo to give the title compound (70.0 mg, 91% yield, TFA) as yellow gum. LC-MS (ESI⁺) m/z 568.3 (M+H)⁺.

### Synthesis of Tert-butyl 2-[4-[[4-[(3S)-3-(1-hydroxy-1-methyl-ethyl)pyrrolidin-1-yl]-5-(trifluoromethyl) pyrimidin-2-yl]amino]-3-methyl-phenyl]sulfonyl-7-azaspiro[3.5]nonane-7-carboxylate (Intermediate QE) and tert-butyl 2-[4-[[4-[(3R)-3-(1-hydroxy-1-methyl-ethyl)pyrrolidin-1-yl]-5-(trifluoromethyl)pyrimidin-2-yl]amino]-3-methyl-phenyl]sulfonyl-7-azaspiro[3.5]nonane-7-carboxylate (Intermediate QF)

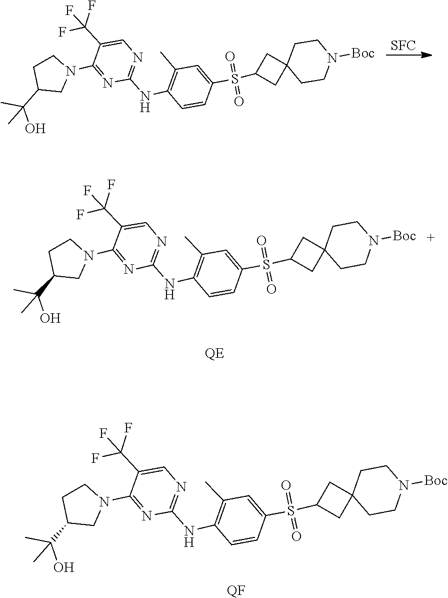

Tert-butyl 2-[4-[[4-[3-(1-hydroxy-1-methyl-ethyl)pyrrolidin-1-yl]-5-(trifluoromethyl) pyrimidin-2-yl]amino]-3-methyl-phenyl]sulfonyl-7-azaspiro[3.5]nonane-7-carboxylate (1.00 g, 1.50 mmol, synthesized via Step 1 of Intermediate QD) was separated by SFC (column: DAICEL CHIRALPAK AD (250 mm*30 mm, 10 um); mobile phase: [CO2-i-PrOH]; B %:35%, isocratic elution mode) to give tert-butyl 2-[4-[[4-[(3S)-3-(1-hydroxy-1-methyl-ethyl)pyrrolidin-1-yl]-5-(trifluoromethyl) pyrimidin-2-yl]amino]-3-methyl-phenyl]sulfonyl-7-azaspiro[3.5]nonane-7-carboxylate (440 mg, 44% yield, ¹H NMR (400 MHz, DMSO-d₆) δ 8.98 (s, 1H), 8.33 (s, 1H), 8.06 (d, J=8.8 Hz, 1H), 7.66 (s, 1H), 7.61-7.59 (m, 1H), 4.42 (s, 1H), 4.09-4.04 (m, 1H), 3.73-3.63 (m, 1H), 3.60-3.54 (m, 1H), 3.51-3.41 (m, 2H), 3.25-3.15 (m, 4H), 2.35 (s, 3H), 2.23-2.15 (m, 1H), 2.10-2.07 (m, 2H), 1.99-1.91 (m, 2H), 1.91-1.85 (m, 1H), 1.83-1.76 (m, 1H), 1.51-1.45 (m, 2H), 1.44-1.40 (m, 2H), 1.37 (s, 9H), 1.11-1.10 (m, 6H). LC-MS (ESI⁺) m/z 668.3 (M+H)+) as yellow solid and tert-butyl 2-[4-[[4-[(3R)-3-(1-hydroxy-1-methyl-ethyl)pyrrolidin-1-yl]-5-(trifluoromethyl)pyrimidin-2-yl]amino]-3-methyl-phenyl]sulfonyl-7-azaspiro[3.5]nonane-7-carboxylate (390 mg, 39% yield, ¹H NMR (400 MHz, DMSO-d₆) δ 8.98 (s, 1H), 8.33 (s, 1H), 8.06 (d, J=8.4 Hz, 1H), 7.66 (d, J=2.0 Hz, 1H), 7.61-7.58 (m, 1H), 4.43 (s, 1H), 4.09-4.05 (m, 1H), 3.67-3.65 (m, 1H), 3.60-3.53 (m, 1H), 3.51-3.42 (m, 2H), 3.25-3.16 (m, 4H), 2.35 (s, 3H), 2.23-2.16 (m, 1H), 2.10-2.07 (m, 2H), 1.99-1.94 (m, 2H), 1.89-1.87 (m, 1H), 1.81-1.76 (m, 1H), 1.51-1.46 (m, 2H), 1.42-1.41 (m, 2H), 1.37 (s, 9H), 1.11-1.10 (m, 6H). LC-MS (ESI⁺) m/z 668.3 (M+H)+) as yellow solid. The absolute stereochemistry of the enantiomers was assigned arbitrarily.

### Synthesis of 2-[(3S)-1-[2-[4-(7-azaspiro[3.5]nonan-2-ylsulfonyl)-2-methyl-anilino]-5-(trifluoromethyl) pyrimidin-4-yl]pyrrolidin-3-yl]propan-2-ol (Intermediate QG)

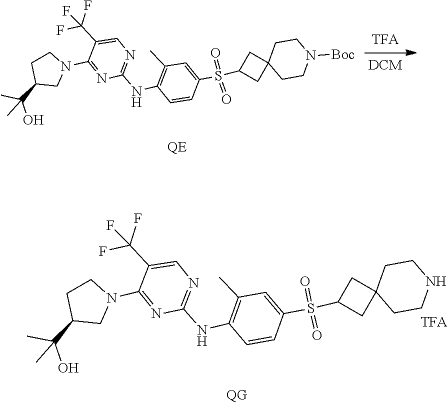 [SMILES: Cc1cc(S(=O)(=O)C2CC3(CCN(C(=O)OC(C)(C)C)CC3)C2)ccc1Nc1ncc(C(F)(F)F)c(N2CC[C@H](C(C)(C)O)C2)n1 ; Cc1cc(S(=O)(=O)C2CC3(CCNCC3)C2)ccc1Nc1ncc(C(F)(F)F)c(N2CC[C@H](C(C)(C)O)C2)n1]

A solution of tert-butyl 2-[4-[[4-[(3S)-3-(1-hydroxy-1-methyl-ethyl)pyrrolidin-1-yl]-5-(trifluoromethyl) pyrimidin-2-yl]amino]-3-methyl-phenyl]sulfonyl-7-azaspiro[3.5]nonane-7-carboxylate (80.0 mg, 119 μmol, Intermediate QE) in DCM (1 mL) and TFA (0.3 mL) was stirred at 25° C. for 0.5 hr. On completion, the mixture was concentrated in vacuo to give the title compound (75.0 mg, 92% yield, TFA) as yellow gum. LC-MS (ESI⁺) m/z 568.3 (M+H)⁺. [1710] 3-[5-(Azetidin-3-yl)-4-fluoro-3-methyl-2-oxo-benzimidazol-1-yl]piperidine-2,6-dione (Intermediate QH)

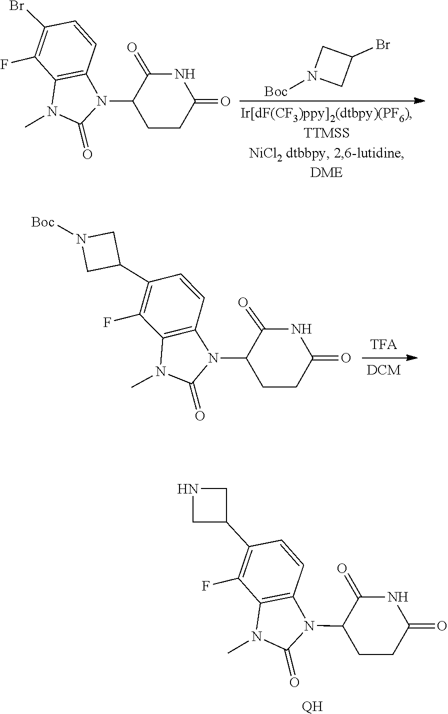

### Step 1—Tert-butyl 3-[1-(2,6-dioxo-3-piperidyl)-4-fluoro-3-methyl-2-oxo-benzimidazol-5-yl]azetidine-1-carboxylate

Ten reactions were run in parallel. To an 15 mL vial equipped with a stir bar was added 3-(5-bromo-4-fluoro-3-methyl-2-oxo-benzimidazol-1-yl)piperidine-2,6-dione (500 mg, 1.40 mmol, synthesized via Step 1 of Intermediate MZ), tert-butyl 3-bromoazetidine-1-carboxylate (430 mg, 1.83 mmol, CAS #1064194-10-0), Ir[dF(CF₃) ppy]₂(dtbpy) (PF₆) (31.5 mg, 28.0 μmol), NiCl₂.dtbbpy (16.7 mg, 42.1 μmol), TTMSS (349 mg, 1.40 mmol), and 2,6-lutidine (300 mg, 2.81 mmol) in DME (10 mL). The vial was sealed and placed under nitrogen. The reaction was stirred and irradiated with a 10 W blue LED lamp (3 cm away), with cooling water to keep the reaction temperature at 25° C. for 14 hrs. On completion, the 10 batches were combined for work-up. The mixture was filtered, diluted with water (30 mL) and extracted with EA (20 mL×3). The combined organic layer was dried over anhydrous Na₂SO₄, filtered and concentrated in vacuo to give the residue. The residue was purified by reverse-phase (0.1% FA condition) to give the title compound (2.10 g, 34% yield) as yellow solid. ¹H NMR (400 MHz, DMSO-d₆) δ 11.12 (s, 1H), 7.12-7.04 (m, 1H), 7.02-6.95 (m, 1H), 5.38 (dd, J=5.6, 12.8 Hz, 1H), 4.25 (t, J=6.8 Hz, 2H), 4.07-3.98 (m, 1H), 3.97-3.90 (m, 2H), 3.48 (d, J=1.6 Hz, 3H), 3.30 (s, 3H), 2.96-2.84 (m, 1H), 2.68-2.60 (m, 2H), 2.06-1.99 (m, 1H), 1.40 (s, 9H). LC-MS (ESI⁺) m/z 377.1 (M−56)⁺.

### Step 2—3-[5-(Azetidin-3-yl)-4-fluoro-3-methyl-2-oxo-benzimidazol-1-yl]piperidine-2,6-dione

To a solution of tert-butyl 3-[1-(2,6-dioxo-3-piperidyl)-4-fluoro-3-methyl-2-oxo-benzimidazol-5-yl] azetidine-1-carboxylate (1.90 g, 4.39 mmol) in DCM (20 mL) was added TFA (500 mg, 4.39 mmol), then the mixture was stirred at 25° C. for 2 hrs. On completion, the mixture was concentrated in vacuo to give the title compound (1.80 g, 91% yield, TFA) as yellow oil. LC-MS (ESI⁺) m/z 333.0 (M+H)+

### Synthesis of 3-[5-[1-[[4-[(2R)-2-aminopropoxy]cyclohexyl]methyl]azetidin-3-yl]-4-fluoro-3-methyl-2-oxo-benzimidazol-1-yl]piperidine-2,6-dione (Intermediate QI)

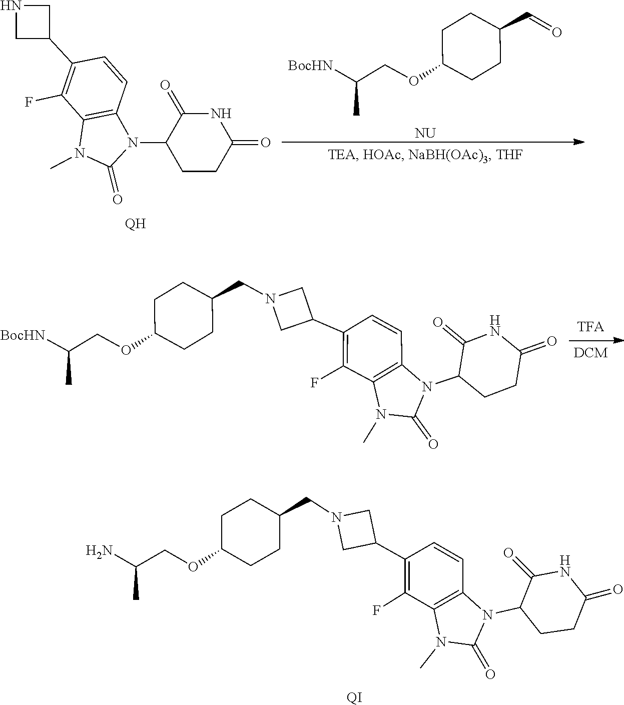

### Step 1—Tert-butyl N-[(1R)-2-[4-[[3-[1-(2,6-dioxo-3-piperidyl)-4-fluoro-3-methyl-2-oxo-benzimidazol-5-yl]azetidin-1-yl]methyl]cyclohexoxy]-1-methyl-ethyl]carbamate

To a solution of 3-[5-(azetidin-3-yl)-4-fluoro-3-methyl-2-oxo-benzimidazol-1-yl]piperidine-2,6-dione (200 mg, 448 μmol, TFA, Intermediate QH) in THF (4 mL) was added TEA (45.3 mg, 448 μmol) until the pH=9. Then tert-butyl N-[(1R)-2-(4-formylcyclohexoxy)-1-methyl-ethyl]carbamate (127 mg, 448 mol, Intermediate NU) was added and AcOH (26.9 mg, 448 mol) was added until pH=6, and the mixture was stirred at −10° C. for 1 hr. Then NaBH(OAc)₃ (237 mg, 1.12 mmol) was added and the mixture was stirred at −10° C. for 0.5 hr. On completion, the mixture was quenched with water (3 mL) at −10° C., and concentrated in vacuo to give the residue. The residue was purified by prep-HPLC (column: Welch Xtimate C18 150*25 mm*5 um; mobile phase: [water (NH₄HCO₃)-MeOH]; gradient: 32%-62% B over 14 min) to give the title compound (110 mg, 40% yield) as a white solid. ¹H NMR (400 MHz, DMSO-d₆) δ 11.11 (s, 1H), 7.11-7.03 (m, 1H), 6.98 (d, J=8.4 Hz, 1H), 6.58 (d, J=7.6 Hz, 1H), 5.37 (dd, J=5.2, 12.8 Hz, 1H), 3.95-3.84 (m, 3H), 3.59-3.49 (m, 2H), 3.47 (d, J=1.2 Hz, 3H), 3.34-3.29 (m, 2H), 3.19-3.10 (m, 2H), 2.95-2.84 (m, 1H), 2.76-2.66 (m, 1H), 2.66-2.58 (m, 1H), 2.47-2.45 (m, 2H), 2.02-1.98 (m, 1H), 1.94-1.92 (m, 2H), 1.76-1.73 (m, 2H), 1.37 (s, 9H), 1.33-1.27 (m, 1H), 1.13-1.03 (m, 2H), 0.98 (d, J=6.6 Hz, 3H), 0.96-0.84 (m, 2H). LC-MS (ESI⁺) m/z 602.5 (M+H)⁺.

### Step 2—3-[5-[1-[[4-[(2R)-2-aminopropoxy]cyclohexyl]methyl]azetidin-3-yl]-4-fluoro-3-methyl-2-oxo-benzimidazol-1-yl]piperidine-2,6-dione

To a solution of tert-butyl N-[(1R)-2-[4-[[3-[1-(2,6-dioxo-3-piperidyl)-4-fluoro-3-methyl-2-oxo-benzimidazol-5-yl]azetidin-1-yl]methyl]cyclohexoxy]-1-methyl-ethyl]carbamate (95.0 mg, 157 mol) in DCM (1.5 mL) wad added TFA (18.0 mg, 157 mol), then the mixture was stirred at 25° C. for 1 hr. On completion, the mixture was concentrated in vacuo to give the title compound (90.0 mg, 92% yield, TFA) as yellow oil. LC-MS (ESI⁺) m/z 502.1 (M+H)⁺.

### Synthesis of 3-[4-(Azetidin-3-yl)-3-methyl-2-oxo-benzimidazol-1-yl]piperidine-2,6-dione (Intermediate QJ)

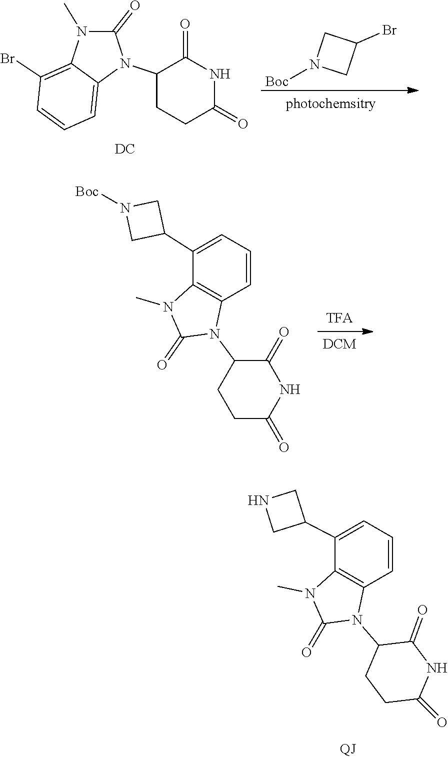

### Step 1—Tert-butyl 3-[1-(2,6-dioxo-3-piperidyl)-3-methyl-2-oxo-benzimidazol-4-yl]azetidine-1-carboxylate

To a solution of 3-(4-bromo-3-methyl-2-oxo-benzimidazol-1-yl)piperidine-2,6-dione (4.05 g, 11.9 mmol, Intermediate DC) and tert-butyl 3-bromoazetidine-1-carboxylate (2.83 g, 11.9 mmol, CAS#1064194-10-0) in DME (210 mL) was added bis[3,5-difluoro-2-[5-(trifluoromethyl)-2-pyridyl]phenyl]iridium (1+); 4-tert-butyl-2-(4-tert-butyl-2-pyridyl)pyridine; hexafluorophosphate (268 mg, 239 mol), 4-tert-butyl-2-(4-tert-butyl-2-pyridyl)pyridine dichloronickel (143 mg, 359 μmol), TTMSS (2.98 g, 11.9 mmol, 3.69 mL) and 2,6-dimethylpyridine (11.5 g, 107 mmol, 12.5 mL). Then the mixture was stirred at 25° C. for 14 hrs under N₂. On completion, the reaction mixture was concentrated in vacuo. The residue was diluted with water (100 mL) and extracted with EA (150 mL×3). The combined organic layers were washed with water (50 mL×3), dried over Na₂SO₄, filtered and concentrated in vacuo. The residue was purified by column chromatography (SiO₂, PE:EA=3:1 to 0:1) to give the title compound (3.61 g, 8.71 mmol, 72% yield) as yellow solid. ¹H NMR (400 MHz, DMSO-d₆) δ 11.32-10.87 (m, 1H), 7.24 (d, J=7.6 Hz, 1H), 7.14-7.03 (m, 2H), 5.38 (dd, J=5.2, 12.4 Hz, 1H), 4.55-4.42 (m, 1H), 4.28 (t, J=8.0 Hz, 2H), 3.97 (d, J=6.0 Hz, 2H), 3.50 (s, 3H), 2.96-2.81 (m, 1H), 2.79-2.56 (m, 3H), 1.41 (s, 9H).

### Step 2—3-[4-(Azetidin-3-yl)-3-methyl-2-oxo-benzimidazol-1-yl]piperidine-2,6-dione

To a solution of tert-butyl 3-[1-(2,6-dioxo-3-piperidyl)-3-methyl-2-oxo-benzimidazol-4-yl]azetidine-1-carboxylate (300 mg, 724 μmol) in DCM (8 mL) was added TFA (50.5 mmol, 3.75 mL), then the mixture was stirred at 25° C. for 1 hr. On completion, the mixture was concentrated in vacuo to give the title compound (300 mg, 97% yield, TFA) as a yellow solid. LC-MS (ESI⁺) m/z 315.0 (M+H)⁺.

### Synthesis of 3-[4-[1-[[4-[(2R)-2-aminopropoxy]cyclohexyl]methyl]azetidin-3-yl]-3-methyl-2-oxo-benzimidazol-1-yl]piperidine-2,6-dione (Intermediate QK)

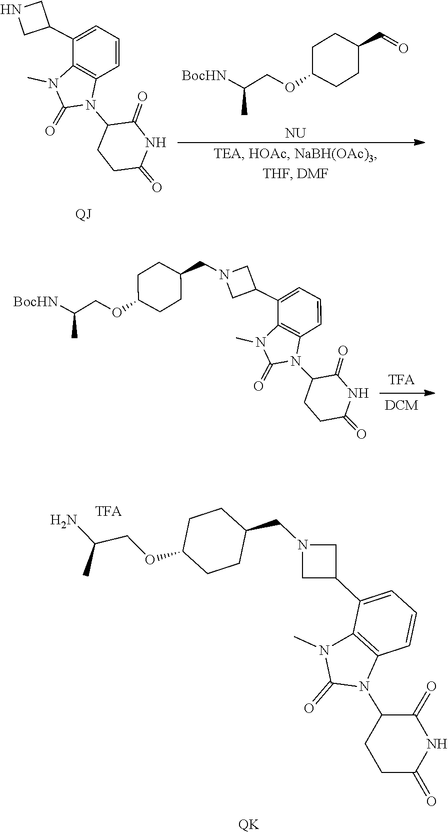

### Step 1—Tert-butyl N-[(1R)-2-[4-[[3-[1-(2,6-dioxo-3-piperidyl)-3-methyl-2-oxo-benzimidazol-4-yl]azetidin-1-yl]methyl]cyclohexoxy]-1-methyl-ethyl]carbamate

To a solution of 3-[4-(azetidin-3-yl)-3-methyl-2-oxo-benzimidazol-1-yl]piperidine-2,6-dione (300 mg, 700 mol, TFA, Intermediate QJ) in THF (2 mL) and DMF (0.5 mL) was added TEA (700 μmol, 97.5 μL) at −10° C. Then tert-butyl N-[(1R)-2-(4-formylcyclohexoxy)-1-methyl-ethyl]carbamate (260 mg, 910 μmol, Intermediate NU) and AcOH (700 mol, 40.1 μL) were added and the mixture was stirred at −10° C. for 0.2 hr. Next, NaBH(OAc)₃ (445 mg, 2.10 mmol) was added and the mixture was stirred at −10° C. for 2 hr. On completion, the mixture was concentrated in vacuo to give the residue. The residue was purified by prep-HPLC (column: Phenomenex luna C18 150*25 mm*10 um; mobile phase: [water (FA)-ACN]; gradient: 14%-44% B over 10 min) to give the title compound (220 mg, 97% yield) as a white solid. ¹H NMR (400 MHz, DMSO-d₆) δ 11.09 (s, 1H), 7.24 (d, J=7.4 Hz, 1H), 7.03-7.12 (m, 2H), 6.53-6.66 (m, 1H), 5.37 (m, 1H), 4.28-4.50 (m, 1H), 3.83-4.07 (m, 2H), 3.60-3.71 (m, 1H), 3.53-3.56 (m, 1H), 3.51 (s, 3H), 3.10-3.21 (m, 3H), 2.79-2.99 (m, 2H), 2.58-2.76 (m, 4H), 2.41-2.48 (m, 1H), 1.91-2.03 (m, 3H), 1.76 (d, J=11.2 Hz, 2H), 1.37 (s, 9H) 1.03-1.14 (m, 2H), 0.98 (d, J=6.8 Hz, 3H), 0.87-0.96 (m, 2H). LC-MS (ESI⁺) m/z 584.3 (M+H)⁺.

### Step 2—3-[4-[1-[[4-[(2R)-2-aminopropoxy]cyclohexyl]methyl]azetidin-3-yl]-3-methyl-2-oxo-benzimidazol-1-yl]piperidine-2,6-dione

To a solution tert-butyl N-[(1R)-2-[4-[[3-[1-(2,6-dioxo-3-piperidyl)-3-methyl-2-oxo-benzimidazol-4-yl] azetidin-1-yl]methyl]cyclohexoxy]-1-methyl-ethyl]carbamate (90.0 mg, 154 μmol) in DCM (1 mL) was added TFA (15.1 mmol, 1.12 mL), then the mixture was stirred at 25° C. for 1 hr. On completion, the mixture was concentrated in vacuo to give the title compound (90.0 mg, 97% yield, TFA) as a yellow solid. LC-MS (ESI⁺) m/z 484.2 (M+H)⁺.

### Synthesis of 4-[[4-[(3S)-3-(1-hydroxy-1-methyl-ethyl)pyrrolidin-1-yl]-5-(trifluoromethyl)pyrimidin-2-yl]amino]-3-methyl-benzenesulfonyl chloride (Intermediate QL)

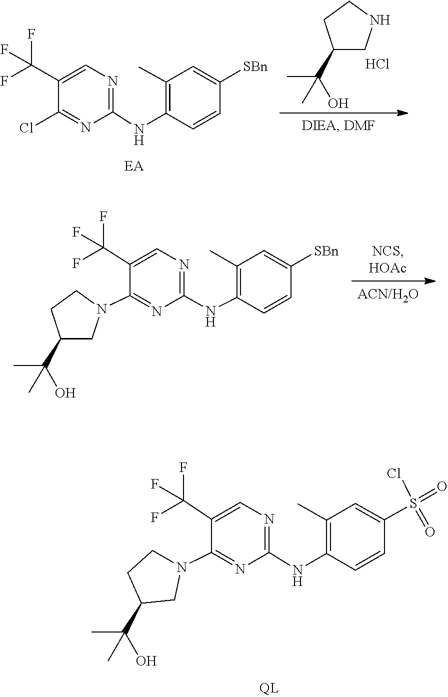

### Step 1—2-[(3S)-1-[2-(4-benzylsulfanyl-2-methyl-anilino)-5-(trifluoromethyl)pyrimidin-4-yl] pyrrolidin-3-yl]propan-2-ol

To a solution of 2-[(3S)-pyrrolidin-3-yl]propan-2-ol (189 mg, 1.46 mmol, CAS #1245645-24-2) in ACN (6 mL) was added TEA (3.66 mmol, 509 μL), then N-(4-benzylsulfanyl-2-methyl-phenyl) -4-chloro-5-(trifluoromethyl)pyrimidin-2-amine (500 mg, 1.22 mmol, Intermediate EA) was added. The mixture was then stirred at 25° C. for 1 hour. On completion, the mixture was diluted with EA (5 mL) and H₂O (20 mL), then extracted with EA (4×5 mL). The combined organic layers were dried anhydrous Na₂SO₄, filtered and concentrated in vacuo to give the residue. The residue was purified by column chromatography (SiO₂, PE:EA=5:1 to 1:1) to give the title compound (500 mg, 82% yield) as a white solid. ¹H NMR (400 MHz, DMSO-d₆) δ 8.73 (s, 1H), 8.24 (s, 1H), 7.48 (d, J=8.4 Hz, 1H), 7.26-7.34 (m, 4H), 7.20-7.25 (m, 1H), 7.18 (d, J=2.0 Hz, 1H), 7.11 (m, 1H), 4.41 (s, 1H), 4.18 (s, 2H), 3.61 (t, J=9.6 Hz, 1H), 3.49-3.55 (m, 1H), 3.37-3.45 (m, 2H), 2.18 (s, 3H), 2.06-2.17 (m, 1H), 1.85 (m, 1H), 1.76 (m, 1H), 1.10 (d, J=5.6 Hz, 6H). LC-MS (ESI⁺) m/z 503.1 (M+H)⁺.

### Step 2—4-[[4-[(3S)-3-(1-hydroxy-1-methyl-ethyl)pyrrolidin-1-yl]-5-(trifluoromethyl)pyrimidin-2-yl]amino]-3-methyl-benzenesulfonyl chloride

To a solution of 2-[(3S)-1-[2-(4-benzylsulfanyl-2-methyl-anilino)-5-(trifluoromethyl)pyrimidin-4-yl] pyrrolidin-3-yl]propan-2-ol (70.0 mg, 139 μmol) in ACN (1.5 mL) was added AcOH (2.45 mmol, 140 L), H₂O (3.89 mmol, 70.0 μL) and NCS (55.8 mg, 418 μmol) in the dark. The mixture was then stirred at 25° C. for 0.5 hr in the dark. On completion, the mixture was concentrated in vacuo to give the title compound (60.0 mg, 90% yield) as a yellow solid. LC-MS (ESI⁺) m/z 478.9 (M+H)⁺.

### Synthesis of 5-Methylpiperidin-3-ol (Intermediate QM)

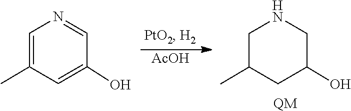

To a solution of 5-methylpyridin-3-ol (3.00 g, 27.5 mmol, CAS #42732-49-0) in AcOH (50 mL) was added PtO₂ (3.12 g, 13.8 mmol) under Ar atmosphere, and then the reaction mixture was degassed and purged with H₂ for 3 times. After that the reaction mixture was stirred at 80° C. for 12 hours under H₂ atmosphere (3.5 Mpa). On completion, the reaction mixture was filtered and concentrated in vacuo to give the title compound (1.50 g, 53% yield) as a black oil.

### Synthesis of 1-(2-((4-((7-Azaspiro[3.5]nonan-2-yl)sulfonyl)-2-methylphenyl)amino)-5-(trifluoromethyl) pyrimidin-4-yl)-3,5-dimethylpiperidin-3-ol (Intermediate QN)

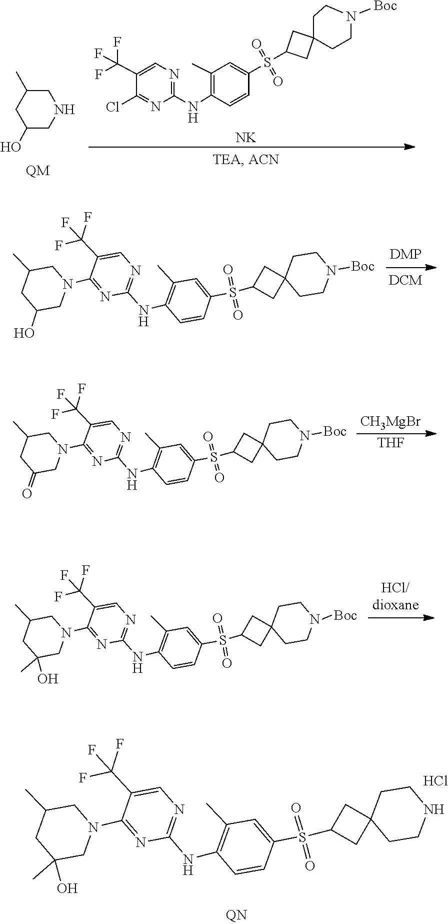

### Step 1—Tert-butyl 2-((4-((4-(3-hydroxy-5-methylpiperidin-1-yl)-5-(trifluoromethyl) pyrimidin-2-yl) 30465862.1 Page 9′74 of 1145 397731-065US (203369) amino)-3-methylphenyl)sulfonyl)-7-azaspiro [3.5]nonane-7-carboxylate

To a solution of tert-butyl 2-[4-[[4-chloro-5-(trifluoromethyl)pyrimidin-2-yl]amino]-3-methyl-phenyl] sulfonyl-7-azaspiro[3.5]nonane-7-carboxylate (1.10 g, 1.91 mmol, Intermediate NK), and 5-methylpiperidin-3-ol (330 mg, 2.87 mmol, Intermediate QM) in ACN (5 mL) was added DIPEA (742 mg, 5.74 mmol), and then the mixture was stirred at 70° C. for 1 hr. On completion, the reaction mixture was partitioned between H₂O (30 mL) and EA (20 mL×3). The organic phase was separated, washed with brine (20 mL×2), dried over anhydrous Na₂SO₄, filtered and concentrated in vacuo to give the title compound (850 mg, 68% yield) as a white solid. LC-MS (ESI⁺) m/z 654.3 (M+H)⁺.

### Step 2—Tert-butyl 2-((3-methyl-4-((4-(3-methyl-5-oxopiperidin-1-yl)-5-(trifluoromethyl)pyrimidin-2-yl)amino)phenyl)sulfonyl)-7-azaspiro[3.5]nonane-7-carboxylate

To a solution of tert-butyl 2-[4-[[4-(3-hydroxy-5-methyl-1-piperidyl)-5-(trifluoromethyl) pyrimidin-2-yl]amino]-3-methyl-phenyl]sulfonyl-7-azaspiro[3.5]nonane-7-carboxylate (850 mg, 1.30 mmol) in DCM (5 mL) was added DMP (1.38 g, 3.25 mmol), and then the mixture was stirred at 15° C. for 5 hrs. On completion, the reaction mixture was partitioned between H₂O (30 mL) and DCM (20 mL×3). The organic phase was separated, washed with brine (20 mL×2), dried over anhydrous Na₂SO₄, filtered and concentrated in vacuo to give the residue. The residue was purified by column chromatography (SiO₂, PE/EA=3/1 to 1/1) to give the title compound (300 mg, 60% yield) as a white solid. LC-MS (ESI⁺) m z 652.4 (M+H)⁺.

### Step 3—Tert-butyl 2-((4-((4-(3-hydroxy-3,5-dimethylpiperidin-1-yl)-5-(trifluoromethyl)pyrimidin-2-yl)amino)-3-methylphenyl)sulfonyl)-7-azaspiro[3.5]nonane-7-carboxylate

To a solution of tert-butyl 2-[3-methyl-4-[[4-(3-methyl-5-oxo-1-piperidyl)-5-(trifluoromethyl) pyrimidin-2-yl]amino]phenyl]sulfonyl-7-azaspiro[3.5]nonane-7-carboxylate (100 mg, 153 μmol) in THF (2 mL) was added methylmagnesium bromide (3 M, 102 μL) at 0° C. dropwise, and then the mixture was stirred at 15° C. for 1 hr. On completion, the reaction mixture was partitioned between H₂O (30 mL) and EA (20 mL×3). The organic phase was separated, washed with brine (20 mL×2), dried over anhydrous Na₂SO₄, filtered and concentrated in vacuo to give the residue. The residue was purified by reserve column (0.1% FA) to give the title compound (24.0 mg, 23% yield) as a white solid. ¹H NMR (400 MHz, CD₃Cl) 6 8.37 (d, J=8.4 Hz, 1H), 8.29 (s, 1H), 7.66-7.60 (m, 2H), 7.16 (s, 1H), 4.34-4.28 (m, 1H), 4.09-3.97 (m, 2H), 3.76-3.64 (m, 2H), 3.29-3.18 (m, 4H), 2.66 (t, J=11.6 Hz, 1H), 2.48 (t, J=12.4 Hz, 1H), 2.34 (s, 3H), 2.29-2.22 (m, 2H), 2.16-2.07 (m, 1H), 2.02-1.90 (m, 3H), 1.58-1.51 (m, 2H), 1.50-1.42 (m, 2H), 1.37 (s, 9H), 1.18 (s, 3H), 0.93 (d, J=6.4 Hz, 3H); LCMS (ESI⁺) m/z 668.4 (M+H)⁺.

### Step 4-1-(2-((4-((7-Azaspiro[3.5]nonan-2-yl)sulfonyl)-2-methylphenyl)amino)-5-(trifluoromethyl) pyrimidin-4-yl)-3,5-dimethylpiperidin-3-ol

To a solution of tert-butyl 2-[4-[[4-(3-hydroxy-3,5-dimethyl-1-piperidyl)-5-(trifluoromethyl) pyrimidin-2-yl]amino]-3-methyl-phenyl]sulfonyl-7-azaspiro[3.5]nonane-7-carboxylate (24.0 mg, 35.9 μmol) in dioxane (1 mL) was added HCl/dioxane (4M, 5 mL), and then the mixture was stirred at 25° C. for 1 hr. On completion, the reaction mixture concentrated in vacuo to give the title compound (20.0 mg, 82% yield) as a black oil. LC-MS (ESI⁺) m/z 568.3 (M+H)⁺.

### Synthesis of 3-(3-Methyl-2-oxo-5-(piperazin-1-yl)-2,3-dihydro-1H-benzo[d]imidazol-1-yl)piperidine-2,6-dione (Intermediate QO)

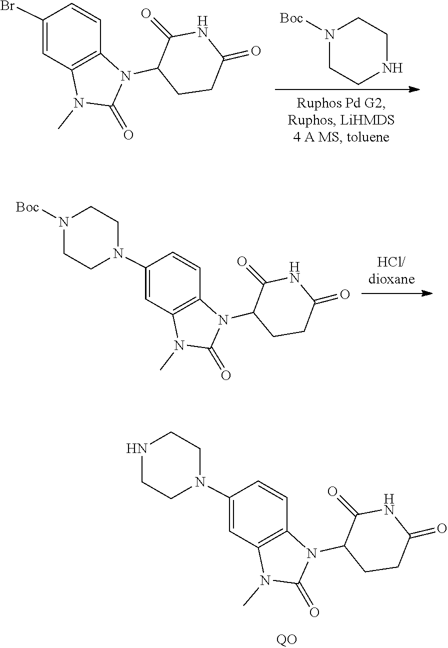 [SMILES: Cn1c(=O)n(C2CCC(=O)NC2=O)c2ccc(Br)cc21 ; CC(C)(C)OC(=O)N1CCNCC1 ; Cn1c(=O)n(C2CCC(=O)NC2=O)c2ccc(N3CCN(C(=O)OC(C)(C)C)CC3)cc21 ; Cn1c(=O)n(C2CCC(=O)NC2=O)c2ccc(N3CCNCC3)cc21]

### Step 1—Tert-butyl 4-(1-(2,6-dioxopiperidin-3-yl)-3-methyl-2-oxo-2,3-dihydro-1H-ben zo[d]imidazol-5-yl)piperazine-1-carboxylate

To a solution of 3-(5-bromo-3-methyl-2-oxo-benzimidazol-1-yl)piperidine-2,6-dione (10 g, 29.6 mmol, Intermediate DA), tert-butyl piperazine-1-carboxylate (8.26 g, 44.4 mmol, CAS #57260-71-6), [2-(2-aminophenyl)phenyl]-chloro-palladium; dicyclohexyl-[2-(2,6-diisopropoxyphenyl)phenyl]phosphane (1.15 g, 1.48 mmol), RuPhos (690 mg, 1.48 mmol) and 4A molecular sieves (2.00 g) in toluene (150 mL) was added LiHMDS (1 M, 88.7 mL) at 0° C. dropwise under N₂ atmosphere. Then the mixture was stirred at 80° C. for 2 hrs under N₂ atmosphere. On completion, the reaction mixture was partitioned between H₂O (400 mL) and EA (200 mL×3). The organic phase was separated, washed with brine (200 mL×2), dried over anhydrous Na₂SO₄, filtered and concentrated in vacuo to give a residue. The residue was purified by reversed-phase (0.1% FA condition) to give the title compound (1.20 g, 9% yield) as a brown solid. LC-MS (ESI⁺) m/z 344.1 (M−99)+.

### Step 2—3-(3-methyl-2-oxo-5-(piperazin-1-yl)-2,3-dihydro-1H-benzo[d]imidazol-1-yl)piperidine-2,6-dione

To a solution of tert-butyl 4-[1-(2,6-dioxo-3-piperidyl)-3-methyl-2-oxo-benzimidazol-5-yl]piperazine-1-carboxylate (500 mg, 1.13 mmol) in dioxane (1 mL) was added HCl/dioxane (4M, 5 mL), and then the mixture was stirred at 25° C. for 1 hr. On completion, the reaction mixture was concentrated in vacuo to give the title compound (420 mg, 98% yield) as a black solid. LC-MS (ESI⁺) m/z 344.3 (M+H)⁺.

### Synthesis of 3-(5-(4-(((1R,4r)-4-((R)-2-aminopropoxy)cyclohexyl)methyl)piperazin-1-yl)-3-methyl-2-oxo-2,3-dihydro-1H-benzo[d]imidazol-1-yl)piperidine-2,6-dione (Intermediate QP)

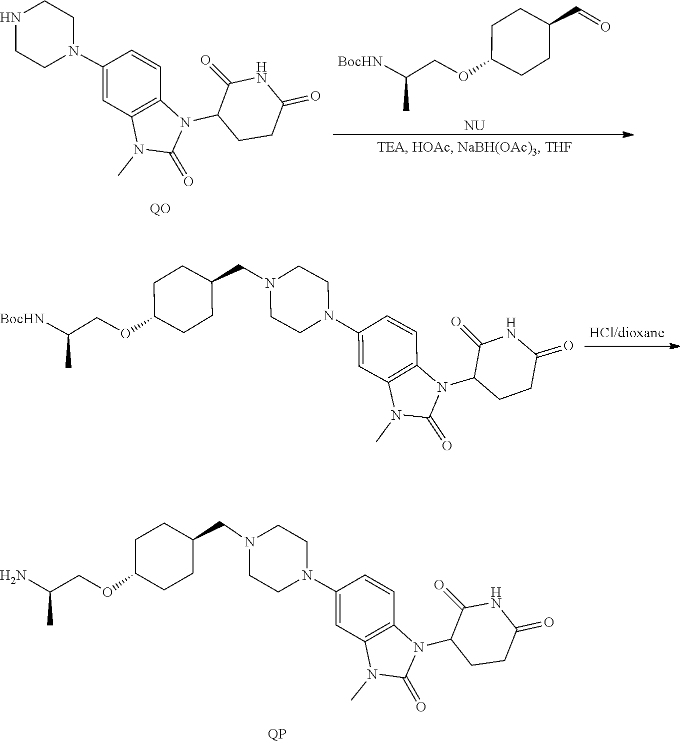 [SMILES: Cn1c(=O)n(C2CCC(=O)NC2=O)c2ccc(N3CCNCC3)cc21 ; C[C@H](CO[C@H]1CC[C@H](C=O)CC1)NC(=O)OC(C)(C)C ; "NU" ; C[C@H](CO[C@H]1CC[C@H](CN2CCN(c3ccc4c(c3)n(C)c(=O)n4C3CCC(=O)NC3=O)CC2)CC1)NC(=O)OC(C)(C)C ; C[C@@H](N)CO[C@H]1CC[C@H](CN2CCN(c3ccc4c(c3)n(C)c(=O)n4C3CCC(=O)NC3=O)CC2)CC1]

### Step 1—Tert-butyl ((2R)-1-(((1r,4R)-4-((4-(1-(2,6-dioxopiperidin-3-yl)-3-methyl-2-oxo-2,3-dihydro-1H-benzo[d]imidazol-5-yl)piperazin-1-yl)methyl)cyclohexyl)oxy)propan-2-yl)carbamate

To a solution of 3-(3-methyl-2-oxo-5-piperazin-1-yl-benzimidazol-1-yl)piperidine-2,6-dione (270 mg, 711 μmol, Intermediate QO) in THF (2 mL) was added TEA (91.2 mg, 902 μmol), and the mixture was stirred at rt for 10 min. Then tert-butyl ((R)-1-(((1r,4R)-4-formylcyclohexyl)oxy)propan-2-yl)carbamate (202 mg, 711 mol, Intermediate NU) and HOAc (28.5 mg, 474 μmol) was added and the reaction mixture was stirred at rt for 1 hr. Next, NaBH(OAc)₃ (402 mg, 1.90 mmol) was added, and the mixture was stirred at rt for 3 hrs. On completion, the reaction mixture was concentrated in vacuo to give a residue. Then the residue was dissolved in ACN and TEA was added to adjust to pH=6 then concentrated in vacuo to give a residue. The residue was purified by reversed-phase (FA condition) to give the title compound (100 mg, 34% yield) as a white solid. LCMS (ESI⁺) m/z 613.4 (M+H)⁺.

### Step 2—3-(5-(4-(((1R,4r)-4-((R)-2-aminopropoxy)cyclohexyl)methyl)piperazin-1-yl)-3-methyl -2-oxo-2,3-dihydro-1H-benzo[d]imidazol-1-yl)piperidine-2,6-dione

To a solution of tert-butyl N-[(1R)-2-[4-[[4-[1-(2,6-dioxo-3-piperidyl)-3-methyl-2-oxo-benzimidazol-5-yl]piperazin-1-yl]methyl]cyclohexoxy]-1-methyl-ethyl]carbamate (100 mg, 163 μmol) in dioxane (1 mL) was added HCl/dioxane (4M, 5 mL), and then the mixture was stirred at 15° C. for 1 hr. On completion, the reaction mixture was concentrated in vacuo to give the title compound (85.0 mg, 94% yield) as a black oil. LC-MS (ESI⁺) m/z 513.3 (M+H)⁺.

### Synthesis of Tert-butyl N-[(1R)-2-(3-formylcyclobutoxy)-1-methyl-ethyl]carbamate (Intermediate QQ)

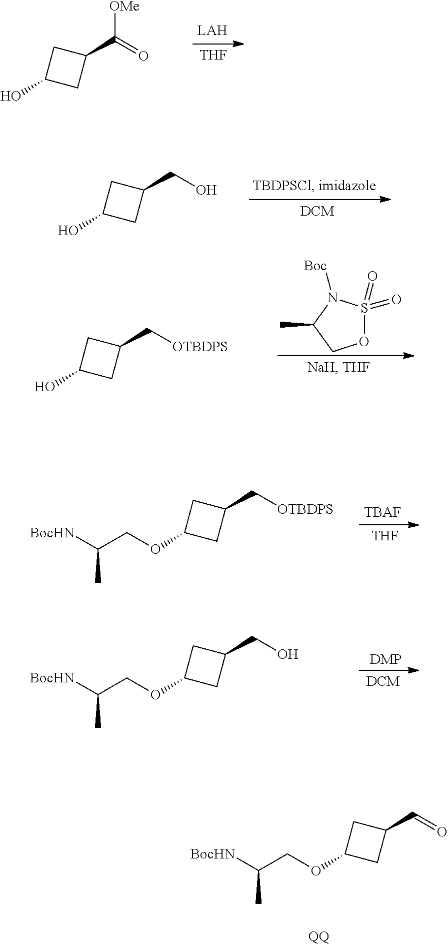

### Step 1—3-(Hydroxymethyl)cyclobutanol

To a solution of methyl 3-hydroxycyclobutanecarboxylate (4.50 g, 34.6 mmol, CAS #1064194-10-0) in THF (90 mL) was added LAH (2.50 M, 20.8 mL) at 0° C., then the mixture was stirred at 25° C. for 2 hrs. On completion, the mixture was quenched with H₂O (1.97 mL), 15% aqueous NaOH (1.97 mL), and H₂O (5.94 mL). Then the mixture was filtered and the filtrate was concentrated in vacuo to give the title compound (3.40 g, 96% yield) as colorless oil. ¹H NMR (400 MHz, DMSO-d₆) δ 4.88 (d, J=6.4 Hz, 1H), 4.47 (t, J=5.2 Hz, 1H), 4.17-4.06 (m, 1H), 3.34 (dd, J=5.2, 7.2 Hz, 2H), 2.11-2.03 (m, 1H), 1.96-1.85 (m, 2H), 1.88-1.79 (m, 2H).

### Step 2-3-[[Tert-butyl(diphenyl)silyl]oxymethyl]cyclobutanol

To a solution of 3-(hydroxymethyl)cyclobutanol (3.30 g, 32.3 mmol) in DCM (40 mL) was added imidazole (2.64 g, 38.8 mmol) and TBDPSCl (9.77 g, 35.5 mmol) at 0° C., then the mixture was stirred at 25° C. for 12 hrs. On completion, the mixture was diluted with water (50 mL) and extracted with DCM (40 mL×3). The combined organic layer was anhydrous Na₂SO₄, filtered and concentrated in vacuo to give the residue. The residue was purified by column chromatography (SiO₂, PE:EA=50:1 to 1:1) to give the title compound (5.50 g, 49% yield) as yellow oil. ¹H NMR (400 MHz, DMSO-d₆) δ 7.62-7.59 (m, 4H), 7.47-7.42 (m, 6H), 4.91 (d, J=6.4 Hz, 1H), 4.18-4.11 (m, 1H), 4.18-4.10 (m, 1H), 3.61 (d, J=6.4 Hz, 2H), 3.32 (s, 1H), 2.27 (dd, J=3.2, 6.4 Hz, 1H), 2.02 (dd, J=3.2, 6.4 Hz, 2H), 1.00 (s, 9H).

### Step 3—Tert-butyl N-[(1R)-2-[3-[[tert-butyl(diphenyl)silyl]oxymethyl]cyclobutoxy]-1-methyl-ethyl]carbamate

To a solution of 3-[[tert-butyl(diphenyl)silyl]oxymethyl]cyclobutanol (1.00 g, 2.94 mmol) in THF (30 mL) was added NaH (234 mg, 5.87 mmol, 60% dispersion in mineral oil) at 0° C., then the mixture was stirred at 0° C. for 0.5 hour. Next, tert-butyl (4R)-4-methyl-2,2-dioxo-oxathiazolidine-3-carboxylate (766 mg, 3.23 mmol, CAS #454248-53-4) was added, and the mixture was stirred at 25° C. for 1 hr. On completion, the mixture was quenched with sat. NH₄Cl (50 mL), diluted with water (80 mL), and extracted with EA (3×60 mL). The combined organic layer was dried over anhydrous Na₂SO₄, filtered and concentrated in vacuo to give the residue. The residue was purified by column chromatography (SiO₂, PE:EA=100:1 to 10:1) to give the title compound (1.80 g, 41% yield) as colorless oil. ¹H NMR (400 MHz, DMSO-d₆) δ 7.80 (d, J=6.8 Hz, 1H), 7.61 (dd, J=2.0, 7.2 Hz, 4H), 7.46-7.39 (m, 6H), 6.62 (d, J=7.2 Hz, 1H), 3.99-3.91 (m, 1H), 3.53 (d, J=6.4 Hz, 1H), 3.65-3.49 (m, 1H), 3.20-3.00 (m, 1H), 3.10-3.00 (m, 1H), 2.32-2.20 (m, 1H), 2.04-1.92 (m, 4H), 1.38-1.34 (m, 9H), 1.02-0.98 (m, 9H), 0.97 (d, J=4.4 Hz, 3H). LC-MS (ESI⁺) m/z 398.2 (M−100)⁺.

### Step 4—Tert-butyl N-[(1R)-2-[3-(hydroxymethyl)cyclobutoxy]-1-methyl-ethyl]carbamate

To a solution of tert-butyl N-[(1R)-2-[3-[[tert-butyl(diphenyl)silyl]oxymethyl]cyclobutoxy]-1-methyl-ethyl]carbamate (1.00 g, 2.01 mmol) in THF (10 mL) was added TBAF (1 M, 3.01 mL) at 0° C. and the mixture was stirred at 25° C. for 2 hrs. On completion, the mixture was diluted with water (10 mL) and extracted with EA (8 mL×3). The combined organic layer was dried over anhydrous Na₂SO₄, filtered and concentrated in vacuo to give the residue. The residue was purified by column chromatography (SiO₂, PE:EA=50:1 to 3:2) to give the title compound (500 mg, 95% yield) as colorless oil. ¹H NMR (400 MHz, DMSO-d₆) δ 6.54 (s, 1H), 4.60-4.51 (m, 1H), 3.95-3.86 (m, J=6.8 Hz, 1H), 3.37 (dd, J=6.0, 6.8 Hz, 2H), 3.20-3.11 (m, 1H), 3.21-2.97 (m, 1H), 2.25-2.10 (m, 1H), 1.99 (s, 1H), 1.96 (dd, J=2.8, 6.4 Hz, 1H), 1.92-1.84 (m, 1H), 1.98-1.81 (m, 1H), 1.37 (s, 9H), 0.99 (dd, J=2.8, 6.8 Hz, 3H).

### Step 5—Tert-butyl N-[(1R)-2-(3-formylcyclobutoxy)-1-methyl-ethyl]carbamate

To a solution of tert-butyl N-[(1R)-2-[3-(hydroxymethyl)cyclobutoxy]-1-methyl-ethyl]carbamate (400 mg, 1.54 mmol) in DCM (10 mL) was added DMP (1.96 g, 4.63 mmol) at 0° C., then the mixture was stirred at 0° C. for 1 hr. On completion, the mixture was quenched with saturated Na₂S₂O₃ (10 mL) and saturated NaHCO₃ (10 mL) at 0° C., diluted with water (20 mL) and extracted with DCM (15 mL×3). The combined organic layers were dried over anhydrous Na₂SO₄, filtered and concentrated in vacuo to give the title compound (300 mg, 75% yield) as yellow oil. LC-MS (ESI⁺) m/z 257.3 (M+H)⁺.

### Synthesis of 3-[4-[4-[[3-[(2R)-2-Aminopropoxy]cyclobutyl]methyl]piperazin-1-yl]-3-methyl-2-oxo-benzimidazol-1-yl]piperidine-2,6-dione (Intermediate QR)

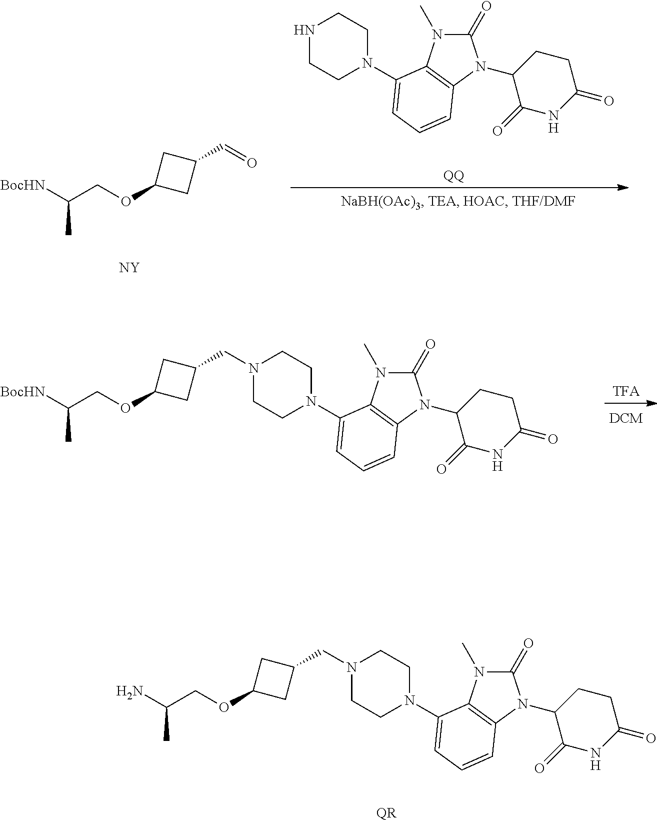

### Step 1—Tert-butyl N-[(1R)-2-[3-[[4-[1-(2,6-dioxo-3-piperidyl)-3-methyl-2-oxo-benzimidazol-4-yl]piperazin-1-yl]methyl]cyclobutoxy]-1-methyl-ethyl]carbamate

To a solution of 3-(3-methyl-2-oxo-4-piperazin-1-yl-benzimidazol-1-yl)piperidine-2,6-dione (180 mg, 393 mol, TFA, Intermediate NY) in THF (1 mL) was added TEA (16.3 μL, 117 μmol) until the pH=8-10. Then tert-butyl N-[(1R)-2-(3-formylcyclobutoxy)-1-methyl-ethyl]carbamate (200 mg, 777 μmol, Intermediate QQ) in DMF (1 mL) and HOAc (22.5 μL, 393 μmol) was added to the mixture and the mixture was stirred at −10° C. for 0.5 hour. After 0.5 hrs, NaBH(OAc)₃ (250 mg, 1.18 mmol) was added and the mixture was stirred at −10° C. for 1 hr. On completion, the mixture was quenched with water (0.5 mL) and filtered to give the filtrate. The filtrate was purified by prep-HPLC (column: Welch Xtimate C18 150*25 mm*5 um; mobile phase: [water (FA)-ACN]; gradient: 3%-33% B over 9 min) to give the title compound (110 mg, 47% yield) as a white solid. ¹HNMR (400 MHz, DMSO-d₆) δ 11.08 (s, 1H), 7.02-6.85 (m, 3H), 6.64 (d, J=8.0 Hz, 1H), 5.35 (dd, J=5.2, 12.4 Hz, 1H), 4.08-3.95 (m, 1H), 3.61 (s, 3H), 3.58-3.53 (m, 1H), 3.47-3.39 (m, 1H), 3.23-3.12 (m, 2H), 3.10-3.01 (m, 2H), 2.99-2.78 (m, 8H), 2.75-2.57 (m, 4H), 2.04-1.92 (m, 4H), 1.38 (s, 9H), 1.03-0.91 (m, 3H). LC-MS (ESI⁺) m/z 585.2 (M+H)⁺.

### Step 2—3-[4-[4-[[3-[(2R)-2-Aminopropoxy]cyclobutyl]methyl]piperazin-1-yl]-3-methyl-2-oxo-benzimidazol-1-yl]piperidine-2,6-dione

To a solution of tert-butyl N-[(1R)-2-[3-[[4-[1-(2,6-dioxo-3-piperidyl)-3-methyl-2-oxo-benzimidazol-4-yl]piperazin-1-yl]methyl]cyclobutoxy]-1-methyl-ethyl]carbamate (100 mg, 171 μmol) in DCM (2 mL) was added TFA (12.7 μL, 171 μmol), then the mixture was stirred at 25° C. for 0.5 hr. On completion, the mixture was concentrated in vacuo to give title compound (100 mg, 97% yield, TFA) as a yellow oil. LC-MS (ESI⁺) m/z 485.1 (M+H)⁺.

### Synthesis of Tert-butyl ((R)-1-((1s,3S)-3-formylcyclobutoxy)propan-2-yl)carbamate (Intermediate QS)

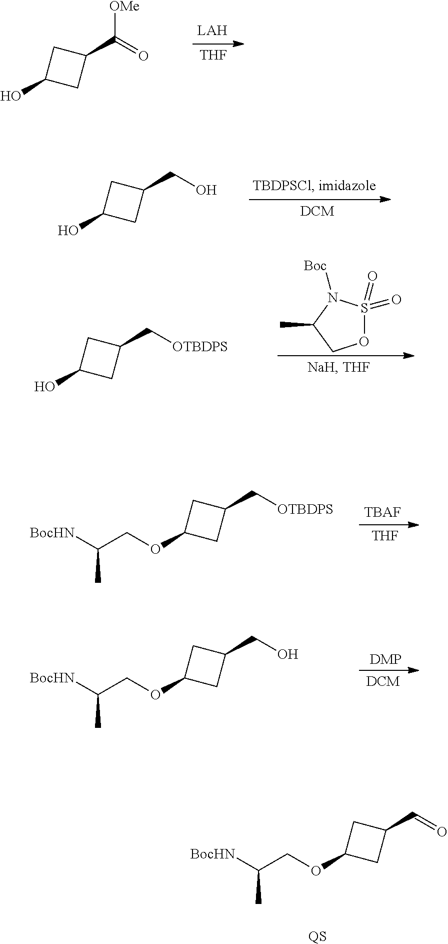

### Step 1—3-(Hydroxymethyl)cyclobutanol

To a solution of methyl 3-hydroxycyclobutanecarboxylate (9.5 g, 73 mmol, CAS #63485-50-7) in THF (5 mL) was added LAH (2.5 M, 43.8 mL) at 0° C., then the mixture was stirred at 20° C. for 2 hrs. On completion, the mixture was quenched with water (4.16 ml), 15% NaOH (4.16 mL) and additional water (12.48 mL) at 0° C. The mixture was dried over Na₂SO₄, filtered and concentrated under reduced pressure to give the title compound (6.7 g, 89% yield) as colorless oil. ¹H NMR (400 MHz, DMSO-d₆) δ 4.83 (d, J=6.4 Hz, 1H), 4.38 (t, J=5.6 Hz, 1H), 3.89 (t, J=7.6 Hz, 1H), 3.32 (s, 1H), 3.30 (s, 1H), 2.22-2.07 (m, 2H), 1.82-1.68 (m, 1H), 1.57-1.41 (m, 2H).

### Step 2-3-[[Tert-butyl(diphenyl)silyl]oxymethyl]cyclobutanol

To a solution of 3-(hydroxymethyl)cyclobutanol (6.7 g, 65.6 mmol) in DMF (60 mL) was added TBDPSCl (16.2 g, 59.0 mmol, 15.1 mL) and imidazole (5.36 g, 78.7 mmol), then the mixture was stirred at 25° C. for 1 hr. On completion, the reaction mixture diluted with water (100 mL) and extracted with EA (200 mL×3). The combined organic layers were washed with water (100 mL×2), dried over Na₂SO₄, filtered and concentrated under reduced pressure. The residue was purified by column chromatography (Silica gel, EA in PE, 15%, v/v) to give the title compound (10.2 g, 45% yield) as colorless oil. ¹H NMR (400 MHz, DMSO-d₆) δ 7.62-7.59 (m, 4H), 7.46-7.42 (m, 6H), 4.91 (d, J=6.0 Hz, 1H), 3.97-3.83 (m, 1H), 3.57 (d, J=5.6 Hz, 2H), 2.19-2.08 (m, 2H), 1.95-1.82 (m, 1H), 1.61-1.49 (m, 2H), 1.00-0.97 (m, 9H).

### Step 3—Tert-butyl N-[(1R)-2-[3-[[tert-butyl(diphenyl)silyl]oxymethyl]cyclobutoxy]-1-methyl-ethyl]carbamate

To a solution of 3-[[tert-butyl(diphenyl)silyl]oxymethyl]cyclobutanol (3 g, 8.81 mmol) and tert-butyl (4R)-4-methyl-2,2-dioxo-oxathiazolidine-3-carboxylate (2.72 g, 11.4 mmol, CAS #454248-53-4) in DMF (25 mL) was added NaH (704 mg, 17.6 mmol, 60% dispersion in mineral oil) at 0° C., then the mixture was stirred at 25° C. for 2 hrs. On completion, the reaction mixture acidified with 2M HCl until the pH=6. The mixture was then diluted with water (50 mL) and extracted with EA (150 mL×3). The combined organic layers were washed with water (100 mL×2), dried over Na₂SO₄, filtered and concentrated under reduced pressure. The residue was purified by column chromatography (Silica gel, EA in PE, 8%, v/v) to give the title compound (2.9 g, 66% yield) as colorless oil. ¹H NMR (400 MHz, DMSO-d₆) δ 7.62-7.57 (m, 4H), 7.48-7.40 (m, 6H), 6.59 (d, J=7.2 Hz, 1H), 3.78 (q, J=7.2 Hz, 1H), 3.58 (d, J=5.2 Hz, 2H), 3.56-3.48 (m, 1H), 3.18 (dd, J=6.0, 9.6 Hz, 1H), 3.04 (dd, J=6.8, 9.2 Hz, 1H), 2.23-2.11 (m, 2H), 2.04-1.94 (m, 1H), 1.76-1.60 (m, 2H), 1.36 (s, 9H), 1.02-0.99 (m, 8H), 0.98-0.94 (m, 4H).

### Step 4—Tert-butyl N-[(1R)-2-[3-(hydroxymethyl)cyclobutoxy]-1-methyl-ethyl]carbamate

To a solution of tert-butyl N-[(1R)-2-[3-[[tert-butyl(diphenyl)silyl]oxymethyl]cyclobutoxy]-1-methyl-ethyl]carbamate (2.9 g, 5.83 mmol) in THF (20 mL) was added TBAF (1 M, 8.74 mL), then the mixture was stirred at 25° C. for 1 hr. On completion, the reaction was concentrated in vacuo. The residue was purified by column chromatography (Silica gel, EA in PE, 50%, v/v) to give the title compound (1.45 g, 96% yield) as colorless oil. ¹H NMR (400 MHz, DMSO-d₆) δ 6.62 (d, J=7.6 Hz, 1H), 4.46 (s, 1H), 3.75 (quin, J=7.2 Hz, 1H), 3.61-3.47 (m, 1H), 3.18 (dd, J=5.6, 9.2 Hz, 1H), 3.03 (dd, J=6.8, 9.2 Hz, 1H), 2.22-2.09 (m, 2H), 1.89-1.77 (m, 1H), 1.59-1.47 (m, 2H), 1.37 (s, 9H), 0.98 (d, J=6.4 Hz, 3H).

### Step 5—Tert-butyl N-[(1R)-2-(3-formylcyclobutoxy)-1-methyl-ethyl]carbamate

To a solution of tert-butyl N-[(1R)-2-[3-(hydroxymethyl)cyclobutoxy]-1-methyl-ethyl]carbamate (400 mg, 1.54 mmol) in DCM (5 mL) was added DMP (785 mg, 1.85 mmol) at 0° C., then the mixture was stirred at 25° C. for 1 hr. On completion, the reaction mixture was quenched by Na₂S₂O₃ (5 mL) and NaHCO₃ (5 mL), and then extracted with DCM (15 mL×3). The combined organic layers were washed with water (10 mL×2), dried over Na₂SO₄, filtered and concentrated in vacuo to give the title compound (390 mg, 98% yield) as colorless oil. ¹H NMR (400 MHz, DMSO-d₆) δ 9.59 (d, J=2.0 Hz, 1H), 8.14-7.95 (m, 1H), 7.90-7.72 (m, 1H), 6.64 (d, J=7.2 Hz, 1H), 3.99-3.85 (m, 1H), 3.59-3.45 (m, 1H), 3.25-3.14 (m, 1H), 3.12-2.99 (m, 1H), 2.82-2.69 (m, 1H), 2.39-2.29 (m, 1H), 2.01-1.92 (m, 1H), 1.37 (s, 9H), 1.02-0.94 (m, 3H).

### Synthesis of 3-[4-[4-[[3-[(2R)-2-aminopropoxy]cyclobutyl]methyl]piperazin-1-yl]-3-methyl-2-oxo-benzimidazol-1-yl]piperidine-2,6-dione (Intermediate QT)

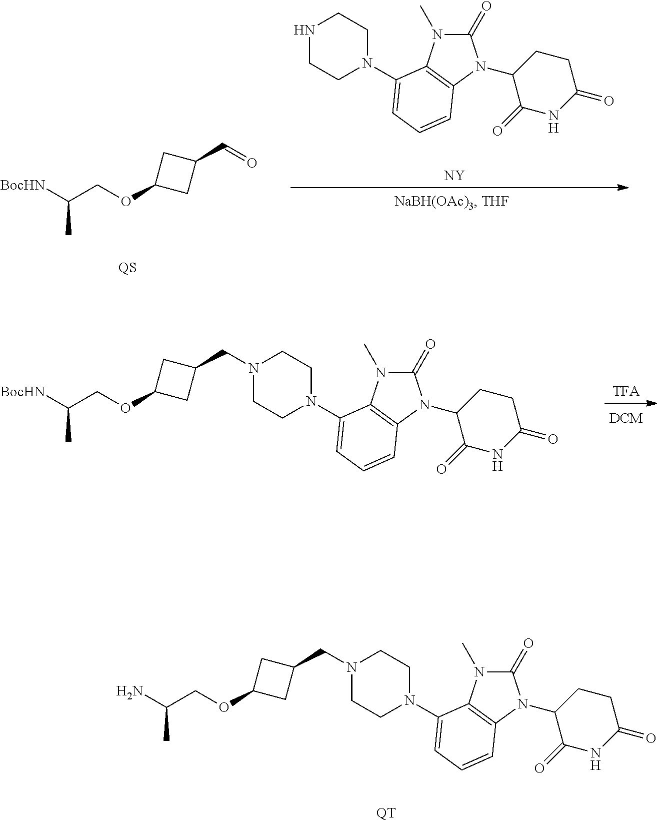

### Step 1—Tert-butyl N-[(1R)-2-[3-[[4-[1-(2,6-dioxo-3-piperidyl)-3-methyl-2-oxo-benzimidazol-4-yl]piperazin-1-yl]methyl]cyclobutoxy]-1-methyl-ethyl]carbamate

To a mixture of 3-(3-methyl-2-oxo-4-piperazin-1-yl-benzimidazol-1-yl)piperidine-2,6-dione (565 mg, 1.24 mmol, TFA, Intermediate NY) in DMF (5 mL) was added TEA (375 mg, 3.71 mmol) at −10° C. until the pH=8. The mixture was stirred at −10° C. for 10 min, then HOAc (742 mg, 12.3 mmol) was added at −10° C. until the pH=6. Subsequently, tert-butyl N-[(1R)-2-(3-formylcyclobutoxy)-1-methyl-ethyl]carbamate (350 mg, 1.36 mmol, Intermediate QS) was added and the mixture was stirred at −10° C. for 20 min. Finally, NaBH(OAc)₃ (786 mg, 3.71 mmol) was added one portion and the mixture was stirred at -10° C. for 1 hr. On completion, the reaction mixture diluted with water (10 mL) and extracted with EA (20 mL×3). The combined organic layers were washed with water (10 mL×2), dried over Na₂SO₄, filtered and concentrated under reduced pressure. The residue was purified by prep-HPLC (column: Welch Xtimate C18 150*25 mm*5 um; mobile phase: [water (FA)-ACN]; gradient: 5%-35% B over 9 min) to give the title compound (100 mg, 13% yield) as white solid. ¹H NMR (400 MHz, DMSO-d₆) δ 11.08 (s, 1H), 8.15 (s, 1H), 7.05-6.79 (m, 3H), 6.70-6.55 (m, 1H), 5.34 (dd, J=5.2, 12.8 Hz, 1H), 3.77 (q, J=7.2 Hz, 1H), 3.61 (s, 3H), 3.57-3.49 (m, 1H), 3.19 (dd, J=6.0, 9.6 Hz, 1H), 3.05 (dd, J=6.8, 9.6 Hz, 1H), 2.95-2.80 (m, 5H), 2.71-2.62 (m, 2H), 2.41 (d, J=6.8 Hz, 3H), 2.37-2.29 (m, 3H), 2.05-1.90 (m, 2H), 1.55-1.43 (m, 2H), 1.38 (s, 9H), 0.99 (d, J=6.8 Hz, 3H).

### Step 2—3-[4-[4-[[3-[(2R)-2-aminopropoxy]cyclobutyl]methyl]piperazin-1-yl]-3-methyl-2-oxo-benzimidazol-1-yl]piperidine-2,6-dione

To a solution of tert-butyl N-[(1R)-2-[3-[[4-[1-(2,6-dioxo-3-piperidyl)-3-methyl-2-oxo-benzimidazol-4-yl]piperazin-1-yl]methyl]cyclobutoxy]-1-methyl-ethyl]carbamate (57 mg, 97.4 μmol) in DCM (1.5 mL) was added TFA (0.3 mL), then the mixture was stirred at 25° C. for 0.5 hr. On completion, the reaction was concentrated under reduced pressure to give the title compound (58 mg, 99% yield, TFA) as a yellow solid. LC-MS (ESI⁺) m/z 485.2 (M+H)⁺.

### Synthesis of Tert-butyl ((1s,3s)-3-(piperazin-1-yl)cyclobutyl)carbamate (Intermediate QU) and tert-butyl ((1r,3r)-3-(piperazin-1-yl)cyclobutyl)carbamate (Intermediate QV)

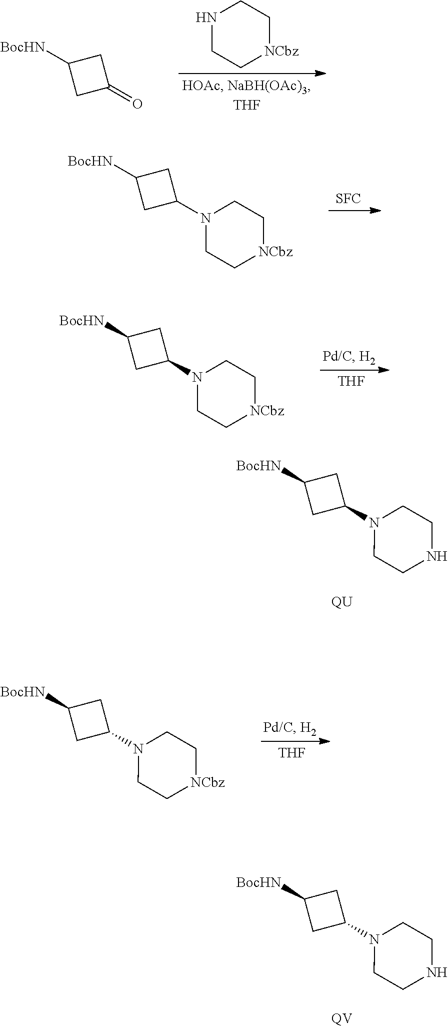

### Step 1—Benzyl 4-[3-(tert-butoxycarbonylamino)cyclobutyl]piperazine-1-carboxylate

A solution of tert-butyl N-(3-oxocyclobutyl)carbamate (10 g, 53.9 mmol, CAS #154748-49-9), benzyl piperazine-1-carboxylate (11.8 g, 53.9 mmol, 10.4 mL, CAS #31166-44-6) and HOAc (1.62 g, 26.9 mmol, 1.55 mL) in THF (200 mL) was stirred at 20° C. for 0.5 hr. Then, NaBH(OAc)₃ (22.8 g, 107 mmol) was added and the mixture was stirred for 16 hrs. On completion, the reaction mixture was quenched by addition of water (100 mL) slowly and extracted with EA (200 mL×2). The combined organic layers were washed with water (150 mL×3), dried over Na₂SO₄, filtered and concentrated in vacuo. The residue was purified by column chromatography (SiO₂, EA in PE, 50% to 100%, v/v) to give the title compound (20 g, 95% yield) as white solid. ¹H NMR (400 MHz, DMSO-d₆) δ 7.50-7.23 (m, 5H), 7.21-6.97 (m, 1H), 5.07 (s, 2H), 3.96-3.56 (m, 1H), 3.37 (s, 4H), 2.78-2.65 (m, 1H), 2.30-2.25 (m, 1H), 2.23-2.14 (m, 4H), 2.14-2.03 (m, 1H), 1.95-1.89 (m, 1H), 1.73-1.59 (m, 1H), 1.36 (d, J=2.4 Hz, 9H); LC-MS (ESI⁺) m z 390.5 (M+H)⁺.

### Step 2—Benzyl 4-[3-(tert-butoxycarbonylamino)cyclobutyl]piperazine-1-carboxylate

Benzyl 4-[3-(tert-butoxycarbonylamino)cyclobutyl]piperazine-1-carboxylate (20.6 g, 52.8 mmol) was separated by SFC (column: DAICEL CHIRALCEL OJ (250 mm*30 mm, 10 um); mobile phase: [CO₂-MeOH (0.1% NH₃H₂O)]; 10% B isocratic elution mode) to give benzyl 4-[3-(tert-butoxycarbonylamino)cyclobutyl]piperazine-1-carboxylate (6.7 g, trans, peak 2, 32% yield) as yellow solid (¹H NMR (400 MHz, DMSO-d₆) δ 7.45-7.24 (m, 5H), 7.16 (d, J=6.8 Hz, 1H), 5.07 (s, 2H), 3.87 (d, J=6.0 Hz, 1H), 3.38 (s, 4H), 2.79-2.66 (m, 1H), 2.21 (t, J=4.4 Hz, 4H), 2.15-2.03 (m, 2H), 1.95-1.88 (m, 2H), 1.36 (s, 9H); LC-MS (ESI⁺) m/z 390.4 (M+H)+) and benzyl 4-[3-(tert-butoxycarbonylamino)cyclobutyl]piperazine-1-carboxylate (11.1 g, cis, peak 1, 53% yield) was obtained as white solid (¹H NMR (400 MHz, DMSO-d₆) δ 7.47-7.23 (m, 5H), 7.04 (d, J=8.0 Hz, 1H), 5.07 (s, 2H), 3.70-3.54 (m, 1H), 3.36 (s, 4H), 2.35-2.23 (m, 3H), 2.18 (t, J=4.8 Hz, 4H), 1.72-1.58 (m, 2H), 1.36 (s, 9H); LC-MS (ESI⁺) m/z 390.4 (M+H)+). 2D-NMR was used to determine the absolute stereochemistry of the diastereomers.

### Step 3—Tert-butyl ((1s,3s)-3-(piperazin-1-yl)cyclobutyl)carbamate

To a solution of benzyl 4-[3-(tert-butoxycarbonylamino)cyclobutyl]piperazine-1-carboxylate (2 g, 5.13 mmol) in THF (40 mL) was added Pd/C (1 g, 5.13 mmol, 10 wt %) under Ar. The suspension was degassed under vacuum and purged with H₂ several times. The mixture was stirred under H₂ (103 mg, 51.3 mmol) (15 psi) at 20° C. for 4 hrs. On completion, the mixture was diluted with THF (50 mL) and filtered. The filtrate was concentrated in vacuo to give the title compound (1.3 g, 99% yield) as white solid. ¹H NMR (400 MHz, DMSO-d₆) δ 7.01 (d, J=8.0 Hz, 1H), 3.68-3.55 (m, 1H), 2.63 (t, J=4.4 Hz, 4H), 2.28-2.20 (m, 3H), 2.11 (s, 4H), 1.62 (d, J=7.6 Hz, 2H), 1.36 (s, 10H).

### Step 4—Tert-butyl ((1r,3r)-3-(piperazin-1-yl)cyclobutyl)carbamate

To a solution of benzyl 4-[3-(tert-butoxycarbonylamino)cyclobutyl]piperazine-1-carboxylate (2 g, 5.13 mmol) in THF (40 mL) was added Pd/C (1 g, 5.13 mmol, 10 wt %) under Ar. The suspension was degassed under vacuum and purged with H₂ several times. The mixture was stirred under H₂ (103 mg, 51.3 mmol) (15 psi) at 20° C. for 4 hrs. On completion, the mixture was diluted with THF (50 mL) and filtered. The filtrate was concentrated in vacuo to give the title compound (1.3 g, 99% yield) as white solid. ¹H NMR (400 MHz, DMSO-d₆) δ 7.15 (d, J=6.8 Hz, 1H), 3.84 (d, J=6.8 Hz, 1H), 2.67 (t, J=4.4 Hz, 5H), 2.26-2.01 (m, 6H), 1.91-1.84 (m, 2H), 1.43-1.32 (m, 10H).

### Synthesis of 3-(4-(4-((4-((1r,3r)-3-aminocyclobutyl)piperazin-1-yl)methyl)piperidin-1-yl)-3-methyl-2-oxo-2,3-dihydro-1H-benzo[d]imidazol-1-yl)piperidine-2,6-dione (Intermediate QW)

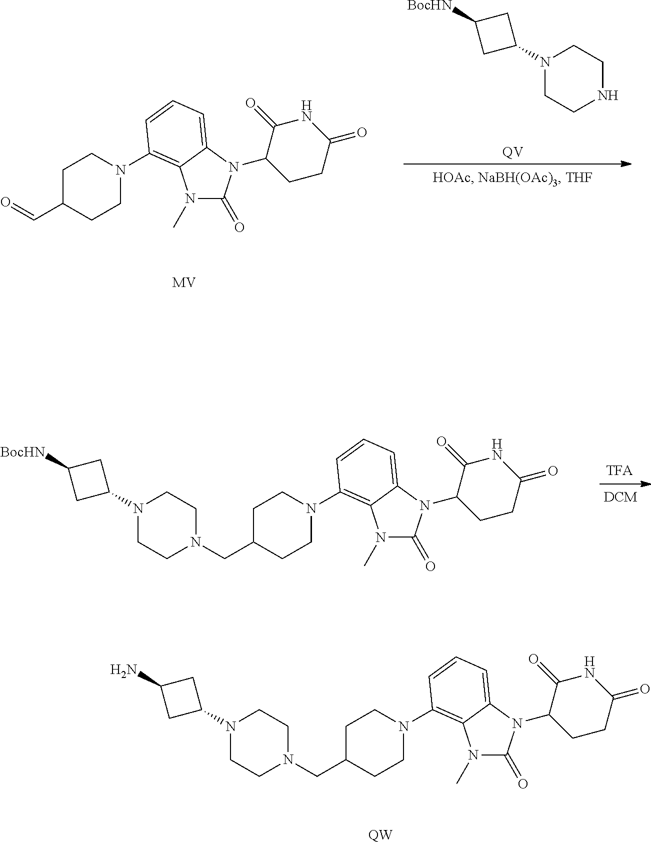

### Step 1—Tert-butyl ((1r,3r)-3-(4-((1-(1-(2,6-dioxopiperidin-3-yl)-3-methyl-2-oxo-2,3-dihydro-1H-benzo[d]imidazol-4-yl)piperidin-4-yl)methyl)piperazin-1-yl)cyclobutyl)carbamate

To a solution of tert-butyl N-(3-piperazin-1-ylcyclobutyl)carbamate (303 mg, 1.19 mmol, Intermediate QV) in THF (3 mL) was added HOAc (32.4 mg, 539 mol, 30.9 μL) at 0° C. Then 1-[1-(2,6-dioxo-3-piperidyl)-3-methyl-2-oxo-benzimidazol-4-yl]piperidine-4-carbaldehyde (400 mg, 1.08 mmol, Intermediate MV) in THF (4 mL) was added to the mixture at 0° C. and the mixture was stirred for 0.5 hr. Next, NaBH(OAc)₃ (457 mg, 2.16 mmol) was added at 0° C. and the mixture was stirred at 0° C. for 0.5 hr. On completion, the reaction mixture was concentrated in vacuo. The residue was purified by prep-HPLC (column: Phenomenex luna C18 150*25 mm*10 um; mobile phase: [water (FA)-ACN]; gradient: 4%-34% B over 10 min) to give the title compound (280 mg, 38% yield) as white solid. ¹H NMR (400 MHz, DMSO-d₆) δ 11.08 (s, 1H), 7.16 (d, J=6.8 Hz, 1H), 7.00-6.93 (m, 1H), 6.91-6.82 (m, 2H), 5.36-5.31 (m, 1H), 3.93-3.80 (m, 1H), 3.61 (s, 3H), 3.10 (d, J=9.6 Hz, 2H), 2.94-2.83 (m, 1H), 2.80-2.56 (m, 6H), 2.47-2.14 (m, 9H), 2.13-2.04 (m, 2H), 2.03-1.96 (m, 1H), 1.94-1.85 (m, 2H), 1.79 (d, J=11.6 Hz, 2H), 1.70-1.55 (m, J=3.6 Hz, 1H), 1.46-1.19 (m, 11H); LC-MS (ESI⁺) m/z 610.3 (M+H)⁺.

### Step 2-3-(4-(4-((4-((1r,3r)-3-aminocyclobutyl)piperazin-1-yl)methyl)piperidin-1-yl)-3-methyl-2-oxo-2,3-dihydro-1H-benzo[d]imidazol-1-yl)piperidine-2,6-dione

To a solution of tert-butyl N-[3-[4-[[1-[1-(2,6-dioxo-3-piperidyl)-3-methyl-2-oxo-benzimidazol-4-yl]-4-piperidyl]methyl]piperazin-1-yl]cyclobutyl]carbamate (140 mg, 229 μmol) in CH₂Cl₂ (2 mL) was added TFA (0.4 mL). The mixture was then stirred at 20° C. for 1 hr. On completion, the mixture was concentrated in vacuo to give the title compound (117 mg, 99% yield) as brown solid. LC-MS (ESI⁺) m z 510.2 (M+H)⁺.

### Synthesis of 3-Methyl-4-((4-(piperidin-1-yl)-5-(trifluoromethyl)pyrimidin-2-yl)amino)benzenesulfonyl chloride (Intermediate QX)

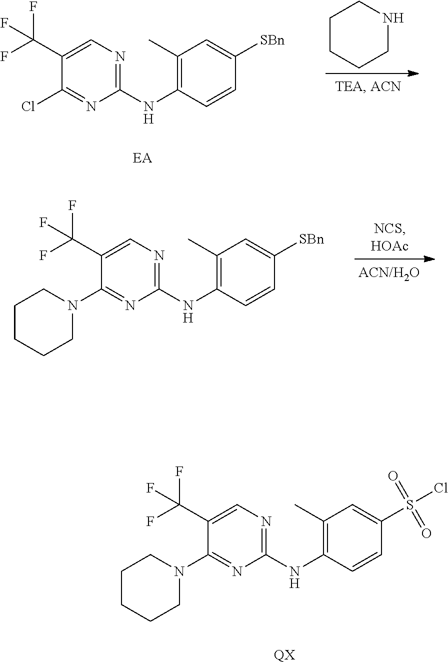

### Step 1—N-(4-(benzylthio)-2-methylphenyl)-4-(piperidin-1-yl)-5-(trifluoromethyl)pyrimidin-2-amine

To a mixture of N-(4-benzylsulfanyl-2-methyl-phenyl)-4-chloro-5-(trifluoromethyl) pyrimidin-2-amine (500 mg, 1.22 mmol, Intermediate EA) and piperidine (135 mg, 1.59 mmol, 156 μL, CAS #110-89-4) in ACN (5 mL) was added TEA (370 mg, 3.66 mmol, 509 μL). The mixture was then stirred at 20° C. for 2 hrs. On completion, the reaction was diluted with H₂O (30 mL) and extracted with EtOAc (60 mL). The combined organic layers were washed with brine (30 mL), dried over with Na₂SO₄, filtered and concentrated under reduced pressure. The residue was purified by flash silica gel chromatography (Silica Flash Column, Eluent of 0˜10% Ethyl acetate/Dichloromethane gradient @ 60 mL/min) to give the title compound (460 mg, 80% yield) as white solid. ¹H NMR (400 MHz, DMSO-d₆) δ 8.92 (s, 1H), 8.28 (s, 1H), 7.42-7.16 (m, 7H), 7.12 (dd, J=2.0, 8.4 Hz, 1H), 4.19 (s, 2H), 3.47-3.37 (m, 4H), 2.17 (s, 3H), 1.65-1.45 (m, 6H); LC-MS (ESI⁺) m/z 459.1 (M+H)⁺.

### Step 2—3-methyl-4-((4-(piperidin-1-yl)-5-(trifluoromethyl)pyrimidin-2-yl)amino)benzenesulfonyl chloride

To a solution of N-(4-benzylsulfanyl-2-methyl-phenyl)-4-(1-piperidyl)-5-(trifluoromethyl) pyrimidin-2-amine (200 mg, 436 μmol) in ACN (2 mL), HOAc (0.2 mL) and H₂O (7.86 mg, 436 μmol, 7.86 μL) was added NCS (174 mg, 1.31 mmol). The mixture was then stirred at 20° C. for 0.5 hr in the dark. On completion, the reaction was diluted with H₂O (20 mL) and extracted with EtOAc (30 mL). The combined organic layers were washed with brine (20 mL), dried over with Na₂SO₄, filtered and concentrated under reduced pressure. The residue was purified by flash silica gel chromatography (ISCO®; 4 g SepaFlash® Silica Flash Column, Eluent of 0˜6% Ethyl acetate/Petroleum ether gradient @ 18 mL/min) to give the title compound (168 mg, 85% yield) as colorless oil. ¹H NMR (400 MHz, DMSO-d₆) δ 9.95 (s, 1H), 8.48 (s, 1H), 7.53 (s, 1H), 7.49-7.40 (m, 2H), 3.63 (s, 4H), 2.26 (s, 3H), 1.62 (s, 6H); LC-MS (ESI⁺) m/z 434.9 (M+H)⁺.

### Synthesis of 3-(Difluoromethyl)piperidine (Intermediate QY)

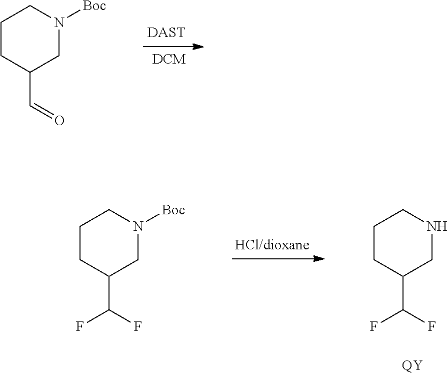

### Step 1—Tert-butyl 3-(difluoromethyl)piperidine-1-carboxylate

To a solution of tert-butyl 3-formylpiperidine-1-carboxylate (5 g, 23.4 mmol, CAS #118156-93-7) in DCM (50 mL) was added DAST (5.67 g, 35.1 mmol, 4.65 mL) at 0° C., then the mixture was stirred at 20° C. for 2 hrs. On completion, the mixture was added into saturated NaHCO₃ solution (120 mL), and extracted with DCM (100 mL×2). The combined organic layers were dried over Na₂SO₄, filtered and the filtrate was concentrated in vacuo. The residue was purified by column chromatography (SiO₂, PE:EA=20:1 to PE:EA=5:1) to give the title compound (2.3 g, 41% yield) as yellow oil. ¹H NMR (400 MHz, DMSO-d₆) δ 6.12-5.79 (m, 1H), 4.01-3.83 (m, 1H), 3.78 (d, J=12.8 Hz, 1H), 2.89-2.65 (m, 2H), 1.97-1.84 (m, 1H), 1.79 (dd, J=4.0, 8.4 Hz, 1H), 1.65 (dd, J=4.4, 7.6 Hz, 1H), 1.43-1.31 (m, 11H).

### Step 2—3-(Difluoromethyl)piperidine

A solution of tert-butyl 3-(difluoromethyl) piperidine-1-carboxylate (2.3 g, 9.78 mmol) in HCl/dioxane (15 mL) was stirred at 20° C. for 0.2 hr. On completion, the reaction was concentrated in vacuo to give the title compound (1.6 g, 95% yield, HCl) as white solid. ¹H NMR (400 MHz, DMSO-d₆) δ 6.27-5.83 (m, 1H), 3.23 (t, J=13.6 Hz, 2H), 2.75 (d, J=10.8 Hz, 2H), 2.45-2.34 (m, 1H), 1.81 (d, J=12.0 Hz, 2H), 1.76-1.65 (m, 1H), 1.42-1.32 (m, 1H).

### Synthesis of N-(4-benzylsulfanyl-2-methyl-phenyl)-4-[(3S)-3-(difluoromethyl)-1-piperidyl]-5-(trifluoromethyl)pyrimidin-2-amine (Intermediate QZ) and N-(4-benzylsulfanyl-2-methyl-phenyl)-4-[(3R)-3-(difluoromethyl)-1-piperidyl]-5-(trifluoromethyl)pyrimidin-2-amine (Intermediate RA)

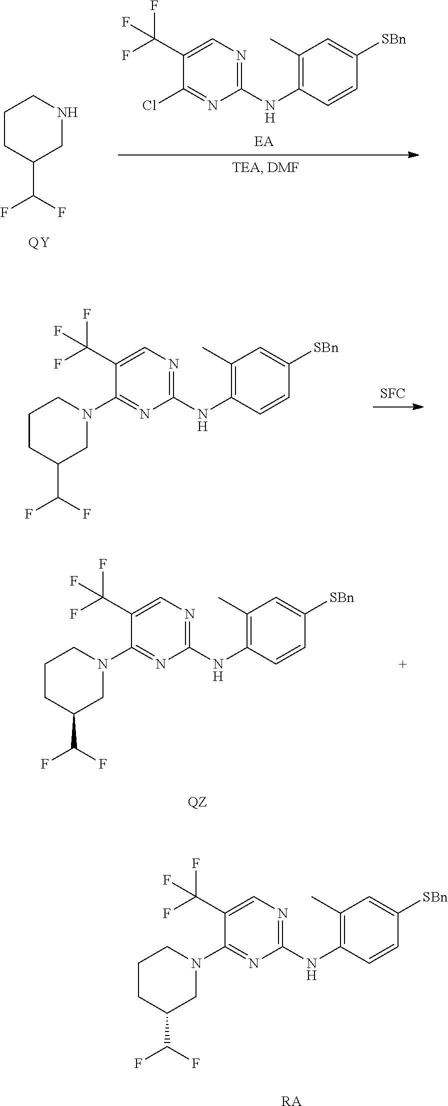

### Step 1—N-(4-benzylsulfanyl-2-methyl-phenyl)-4-[3-(difluoromethyl)-1-piperidyl]-5-(trifluoromethyl) pyrimidin-2-amine

To a solution of 3-(difluoromethyl)piperidine (1.26 g, 7.32 mmol, HCl, Intermediate QY) and N-(4-benzylsulfanyl-2-methyl-phenyl)-4-chloro-5-(trifluoromethyl)pyrimidin-2-amine (2 g, 4.88 mmol, Intermediate EA) in DMF (20 mL) was added TEA (1.48 g, 14.6 mmol, 2.04 mL), then the mixture was stirred at 20° C. for 2 hrs. On completion, the reaction was diluted with EA (100 mL) and washed with water (100 mL×4). The organic layer was dried over Na₂SO₄, filtered and the filtrate was concentrated in vacuo. The residue was purified by column chromatography (SiO₂, PE:EA=20:1 to PE:EA=3:1) to give the title compound (2.1 g, 84% yield) as yellow solid. ¹H NMR (400 MHz, DMSO-d₆) δ 9.02 (s, 1H), 8.33 (s, 1H), 7.39-7.33 (m, 3H), 7.32-7.28 (m, 2H), 7.25-7.20 (m, 2H), 7.13 (dd, J=2.0, 8.4 Hz, 1H), 6.05-5.76 (m, 1H), 4.20 (s, 2H), 4.05 (d, J=12.8 Hz, 1H), 3.88 (d, J=13.4 Hz, 1H), 2.95-2.86 (m, 2H), 2.17 (s, 3H), 2.08-1.99 (m, 1H), 1.86-1.80 (m, 1H), 1.72 (d, J=12.0 Hz, 1H), 1.49-1.39 (m, 2H).

### Step 2—N-(4-benzylsulfanyl-2-methyl-phenyl)-4-[(3S)-3-(difluoromethyl)-1-piperidyl]-5-(trifluoromethyl)pyrimidin-2-amine and N-(4-benzylsulfanyl-2-methyl-phenyl)-4-[(3R)-3-(difluoromethyl)-1-piperidyl]-5-(trifluoromethyl)pyrimidin-2-amine

N-(4-benzylsulfanyl-2-methyl-phenyl)-4-[3-(difluoromethyl)-1-piperidyl]-5-(trifluoromethyl) pyrimidin-2-amine (2.1 g) was separated by SFC (column: DAICEL CHIRALPAK AD (250 mm*30 mm, 10 um); mobile phase: [CO₂-EtOH (0.1% NH₃H₂O)]; B %: 20%, isocratic elution mode) to give N-(4-benzylsulfanyl-2-methyl-phenyl)-4-[(3S)-3-(difluoromethyl)-1-piperidyl]-5-(trifluoromethyl)pyrimidin-2-amine (600 mg, 28% yield, peak 1) as white solid (¹H NMR (400 MHz, DMSO-d₆) δ 9.01 (s, 1H), 8.33 (s, 1H), 7.42-7.32 (m, 3H), 7.30 (t, J=7.6 Hz, 2H), 7.26-7.19 (m, 2H), 7.15-7.10 (m, 1H), 5.90 (d, J=4.4 Hz, 1H), 4.20 (s, 2H), 4.05 (d, J=12.8 Hz, 1H), 3.88 (d, J=13.2 Hz, 1H), 2.97-2.84 (m, 2H), 2.17 (s, 3H), 2.11-1.96 (m, 1H), 1.88-1.79 (m, 1H), 1.70 (s, 1H), 1.44 (d, J=10.8 Hz, 2H); LC-MS (ESI⁺) m z 509.1 (M+H)+) and N-(4-benzylsulfanyl-2-methyl-phenyl)-4-[(3R)-3-(difluoromethyl)-1-piperidyl]-5-(trifluoromethyl) pyrimidin-2-amine (380 mg, 18% yield, peak 2) as yellow solid (¹H NMR (400 MHz, DMSO-d₆) δ 9.08-8.98 (m, 1H), 8.33 (s, 1H), 7.40-7.32 (m, 3H), 7.32-7.27 (m, 2H), 7.26-7.19 (m, 2H), 7.13 (dd, J=2.0, 8.4 Hz, 1H), 6.06-5.75 (m, 1H), 4.20 (s, 2H), 4.05 (d, J=12.4 Hz, 1H), 3.88 (d, J=13.2 Hz, 1H), 2.96-2.84 (m, 2H), 2.17 (s, 3H), 2.10-1.98 (m, 1H), 1.87-1.79 (m, 1H), 1.72 (d, J=11.6 Hz, 1H), 1.51-1.37 (m, 2H); LC-MS (ESI⁺) m/z 509.2 (M+H)+). Absolute stereochemistry of the enantiomers was assigned arbitrarily.

### Synthesis of 4-[[4-[3-(difluoromethyl)-1-piperidyl]-5-(trifluoromethyl)pyrimidin-2-yl]amino]-3-methyl-benzenesulfonyl chloride (Intermediate RB)

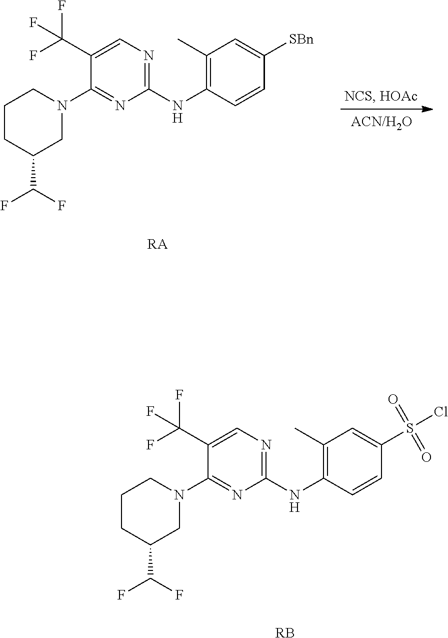

To a solution of N-(4-benzylsulfanyl-2-methyl-phenyl)-4-[(3R)-3-(difluoromethyl)-1-piperidyl]-5-(trifluoromethyl)pyrimidin-2-amine (100 mg, 196 μmol, Intermediate RA) in ACN (2 mL) and AcOH (0.2 mL) was added H₂O (3.54 mg, 196 μmol, 3.54 μL) and NCS (78.7 mg, 589 mol) in the dark. Then the mixture was stirred at 20° C. for 0.5 hr. On completion, the reaction mixture was diluted with EA (10 mL) and washed with water (10 mL). The organic layer was dried over Na₂SO₄, filtered and concentrated in vacuo. The residue was purified by column chromatography (SiO₂, PE:EA=10:1 to PE:EA=1:1) to give the title compound (90 mg, 94% yield) as yellow oil. LC-MS (ESI⁺) m/z 485.0 (M+H)⁺.

### Synthesis of 4-[[4-[(3S)-3-(difluoromethyl)-1-piperidyl]-5-(trifluoromethyl)pyrimidin-2-yl]amino]-3-methyl-benzenesulfonyl chloride (Intermediate RC

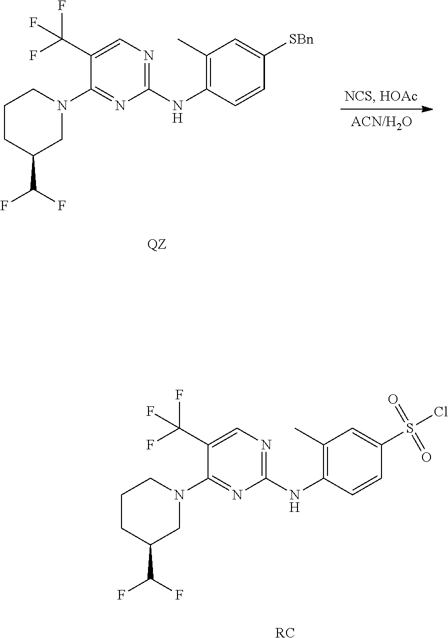

To a solution of N-(4-benzylsulfanyl -2-methyl-phenyl)-4-[(3S) -3-(difluoromethyl) -1-piperidyl]-5-(trifluoromethyl)pyrimidin-2-amine (120 mg, 235 μmol, Intermediate QZ) in ACN (2 mL), HOAc (0.2 mL) and H₂O (0.01 mL) was added NCS (94.5 mg, 707 mol). The mixture was then stirred at 25° C. for 0.5 hr in the dark. On completion, the mixture was concentrated in vacuo. The residue was purified by silica gel column chromatography (SiO₂, PE:EA=10:1 to 1:1) to give the title compound (80 mg, 70% yield) as yellow oil. LC-MS (ESI⁺) m/z 484.9 (M+H)⁺.

### Synthesis of Azetidin-3-ylmethanol (Intermediate RD)

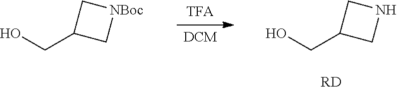

A mixture of tert-butyl 3-(hydroxymethyl)azetidine-1-carboxylate (3 g, 16.02 mmol, CAS#142253-56-3) and TFA (7.68 g, 67.3 mmol, 5 mL) in DCM (20 mL) was stirred at 25° C. for 1 hr. On completion, the reaction was concentrated in vacuo to give the title compound (3.22 g, 100% yield, TFA) as colorless oil.

### Synthesis of [1-[1-[1-[(4-Methoxyphenyl)methyl]-2,6-dioxo-3-piperidyl]-3-methyl-2-oxo-benzimidazo 1-4-yl]azetidin-3-yl]methyl methanesulfonate (Intermediate RE)

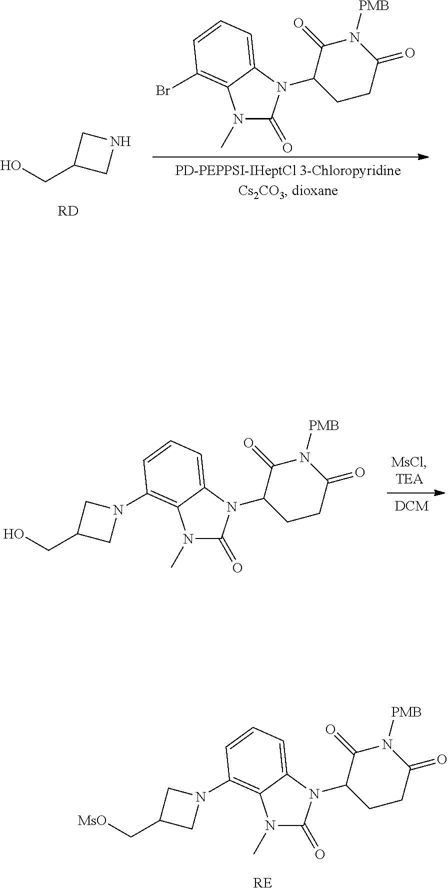

### Step 1—3-[4-[3-(Hydroxymethyl)azetidin-1-yl]-3-methyl-2-oxo-benzimidazol-1-yl]-1-[(4-methoxy phenyl)methyl]piperidine-2,6-dione

To a solution of 3-(4-bromo-3-methyl-2-oxo-benzimidazol-1-yl)-1-[(4-methoxyphenyl) methyl]piperidine-2,6-dione (450 mg, 981 μmol, synthesized via Steps 1-4 of Intermediate DC), azetidin-3-ylmethanol (296 mg, 1.47 mmol, TFA, Intermediate RD) and Cs₂CO₃ (1.28 g, 3.93 mmol) in dioxane (5 mL) was added 1,3-bis[2,6-bis(1-propylbutyl)phenyl]-4,5-dichloro-2H-imidazol-1-ium-2-ide; 3-chloropyridine; dichloropalladium (95.5 mg, 98.1 μmol) under N₂. The reaction was stirred at 100° C. for 16 hrs under N₂. On completion, the reaction was diluted with EA (20 mL). The organic washed with water (20 mL×2), dried over with Na₂SO₄ and concentrated in vacuo. The residue was purified by column chromatography (SiO₂, DCM/EA=1/0 to 1/4) to give the title compound (442 mg, 96% yield) as yellow solid. ¹H NMR (400 MHz, DMSO-d₆) δ 7.20 (d, J=8.4 Hz, 2H), 6.92-6.87 (m, 1H), 6.85 (d, J=8.8 Hz, 2H), 6.63 (d, J=8.0 Hz, 2H), 5.47 (dd, J=5.2, 12.8 Hz, 1H), 4.85-4.79 (m, 1H), 4.77-4.72 (m, 2H), 3.85 (m, J=3.6, 7.2 Hz, 2H), 3.72 (s, 3H), 3.63-3.60 (m, 2H), 3.60-3.57 (m, 2H), 3.56 (s, 3H), 3.51-3.43 (m, 1H), 3.09-2.99 (m, 1H), 2.83-2.77 (m, 1H), 2.72-2.65 (m, 2H), 2.05-2.00 (m, 1H); LC-MS (ESI⁺) m z 465.2 (M+H)⁺.

### Step 2—[1-[1-[1-(4-Methoxyphenyl)methyl]-2,6-dioxo-3-piperidyl]-3-methyl-2-oxo-benzimidazo 1-4-yl]azetidin-3-yl]methyl methanesulfonate

To a solution of 3-[4-[3-(hydroxymethyl)azetidin-1-yl]-3-methyl-2-oxo-benzimidazol-1-yl]-1-[(4-methoxyphenyl)methyl]piperidine-2,6-dione (640 mg, 1.38 mmol) and TEA (418 mg, 4.13 mmol, 575 μL) in DCM (7 mL) was added MsCl (510 mg, 4.45 mmol, 344 μL) at 0° C. The reaction was stirred at 0° C. for 1 hr. On completion, the reaction was quenched with water at 0° C. The reaction was diluted with DCM (20 mL). The organic layer was washed with water (20 mL×2), dried over with Na₂SO₄ and concentrated in vacuo to give the title compound (747 mg, 99% yield) as yellow oil. ¹H NMR (400 MHz, DMSO-d₆) δ 7.21 (d, J=8.8 Hz, 2H), 6.94-6.89 (m, 1H), 6.85 (d, J=8.8 Hz, 2H), 6.69 (d, J=8.0 Hz, 1H), 5.42-5.51 (m, J=5.6, 12.8 Hz, 1H), 4.85-4.70 (m, 2H), 4.47 (d, J=6.8 Hz, 2H), 3.97-3.90 (m, 2H), 3.72 (s, 3H), 3.65-3.60 (m, 2H), 3.58 (s, 3H), 3.23 (s, 3H), 3.09-3.03 (m, 1H), 3.02-2.91 (m, 2H), 2.84-2.77 (m, 1H), 2.74-2.66 (m, 1H), 2.06-1.99 (m, 1H); LC-MS (ESI⁺) m/z 543.2 (M+H)⁺.

### Synthesis of 3-[4-[3-[[4-(4-Aminocyclohexyl)piperazin-1-yl]methyl]azetidin-1-yl]-3-methyl-2-oxo-benzimidazol-1-yl]piperidine-2,6-dione (Intermediate RF)

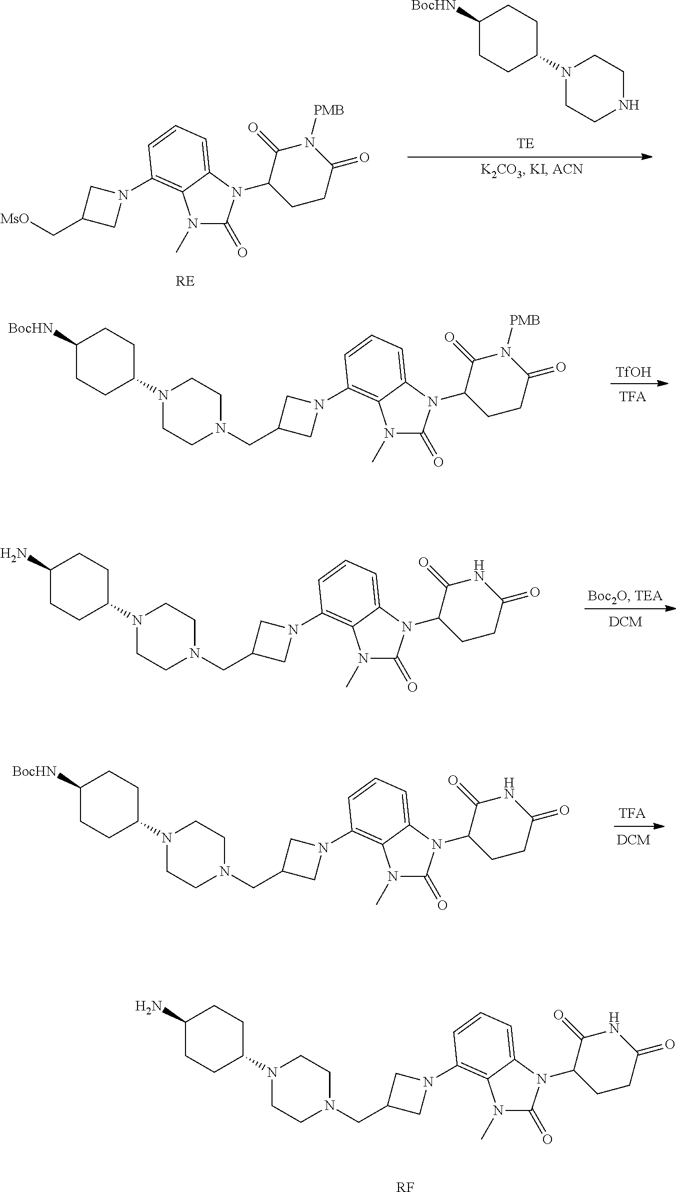

### Step 1—Tert-butyl N-[4-[4-[[1-[1-[1-[(4-methoxyphenyl)methyl]-2,6-dioxo-3-piperidyl]-3-methyl-2-oxo-benzimidazol-4-yl]azetidin-3-yl]methyl]piperazin-1-yl]cyclohexyl]carbamate

To a solution of [1-[1-[1-[(4-methoxyphenyl)methyl]-2,6-dioxo-3-piperidyl]-3-methyl-2-oxo-benzimidazol-4-yl]azetidin-3-yl]methyl methanesulfonate (795 mg, 1.47 mmol, Intermediate RE), tert-butyl N-(4-piperazin-1-ylcyclohexyl)carbamate (539 mg, 1.90 mmol, Intermediate TE) and K₂CO₃ (607 mg, 4.40 mmol) in ACN (15 mL) was added KI (243 mg, 1.47 mmol). The reaction was then stirred at 60° C. for 48 hrs. On completion, the reaction was diluted with EA (50 mL). The organic layer was washed with water (50 mL×2), dried over Na₂SO₄ and concentrated in vacuo. The residue was purified by prep-HPLC (column: Phenomenex luna C18 150*25 mm*10 um; mobile phase: [water (FA)-CAN]; gradient: 15%-45% B over 10 min) to give the title compound (500 mg, 46% yield) as a white solid. ¹H NMR (400 MHz, DMSO-d₆) δ 8.15 (s, 1H), 7.20 (d, J=8.4 Hz, 2H), 6.88-6.82 (m, 2H), 6.71 (d, J=7.6 Hz, 1H), 6.63 (d, J=8.0 Hz, 2H), 5.55-5.40 (m, 1H), 4.87-4.70 (m, 2H), 3.97-3.88 (m, 2H), 3.72 (s, 3H), 3.48 (t, J=4.8 Hz, 2H), 3.22-2.96 (m, 3H), 2.91-2.64 (m, 6H), 2.59 (d, J=6.8 Hz, 6H), 2.43 (s, 3H), 2.26 (t, J=10.4 Hz, 1H), 2.07-1.94 (m, 1H), 1.80 (d, J=9.6 Hz, 4H), 1.37 (s, 9H), 1.24-1.08 (m, 4H); LC-MS (ESI⁺) m/z 730.4 (M+H)⁺.

### Step 2—3-[4-[3-[[4-(4-Aminocyclohexyl)piperazin-1-yl]methyl]azetidin-1-yl]-3-methyl-2-oxo-benzimidazol-1-yl]piperidine-2,6-dione

To a solution of tert-butyl N-[4-[4-[[1-[1-[1-[(4-methoxyphenyl)methyl]-2,6-dioxo-3-piperidyl]-3-methyl-2-oxo-benzimidazol-4-yl]azetidin-3-yl]methyl]piperazin-1-yl]cyclohexyl]carbamate (200 mg, 274 μmol) in TFA (1.5 mL) was added TfOH (848 mg, 5.65 mmol, 0.5 mL). The reaction was then stirred at 70° C. for 1 hr. On completion, the reaction was concentrated in vacuo to give the title compound (170 mg, 99% yield, TFA) as brown oil. LC-MS (ESI⁺) m/z 510.2 (M+H)⁺.

### Step 3—Tert-butyl N-[4-[4-[[1-[1-(2,6-dioxo-3-piperidyl)-3-methyl-2-oxo-benzimidazol-4-yl]azetidin-3-yl]methyl]piperazin-1-yl]cyclohexyl]carbamate

To a solution of 3-[4-[3-[[4-(4-aminocyclohexyl)piperazin-1-yl]methyl]azetidin-1-yl]-3-methyl 2-oxo-benzimidazol-1-yl]piperidine-2,6-dione (170 mg, 272. pmol, TFA) in DCM (1.5 mL) was added TEA (82.7 mg, 817 μmol, 113 μL) and Boc₂O (89.2 mg, 408 μmol, 93.9 μL). The reaction was stirred at 25° C. for 1 hr. On completion, the reaction was diluted with EA (10 mL). The organic layer was washed with water (10 mL×2), dried over Na₂SO₄ and concentrated in vacuo. The crude product was purified by reversed-phase HPLC (0.1% FA condition) to give the title compound (166 mg, 99% yield) as yellow oil. LC-MS (ESI⁺) m/z 610.5 (M+H)⁺.

### Step 4—3-[4-[3-[[4-(4-Aminocyclohexyl)piperazin-1-yl]methyl]azetidin-1-yl]-3-methyl-2-oxo-benzimidazol-1-yl]piperidine-2,6-dione

To a solution of tert-butyl N-[4-[4-[[1-[1-(2,6-dioxo-3-piperidyl)-3-methyl-2-oxo-benzimidazol -4-yl]azetidin-3-yl]methyl]piperazin-1-yl]cyclohexyl]carbamate (166 mg, 272 mol) in DCM (1.5 mL) was added TFA (767 mg, 6.73 mmol, 0.5 mL). The reaction was then stirred at 25° C. for 1 hr. On completion, the reaction was concentrated in vacuo to give the title compound (169 mg, 99% yield, TFA) as yellow oil. LC-MS (ESI⁺) m/z 510.3 (M+H)⁺.

### Synthesis of 1-[5-Fluoro-1-[1-[(4-methoxyphenyl)methyl]-2,6-dioxo-3-piperidyl]-3-methyl-2-oxo-benzimidazol-4-yl]piperidine-4-carbaldehyde (Intermediate RG)

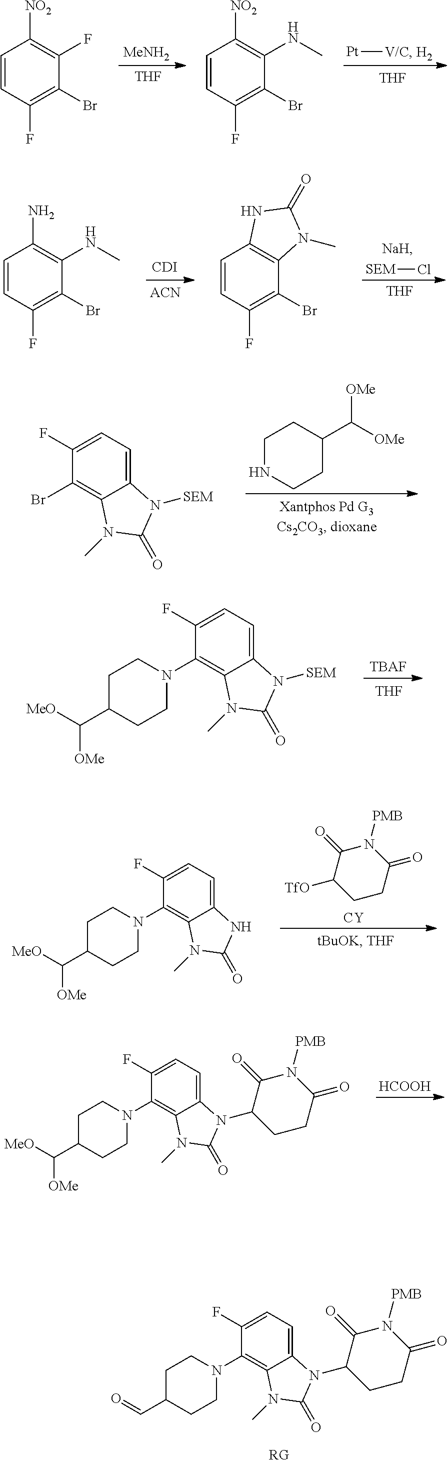

### Step 1—2-Bromo-3-fluoro-N-methyl-6-nitro-aniline

A solution of 2-bromo-1,3-difluoro-4-nitro-benzene (10.0 g, 42.0 mmol, CAS #103977-78-2) in THF (100 mL) saturated with MeNH₂ (2.00 M, 31.5 mL) was stirred at 60° C. for 5 hrs in a sealed tube. Then additional MeNH₂ (2.00 M, 10.5 mL) was added, and the mixture was stirred at 60° C. for 2 hrs in a sealed tube. On completion, the mixture was concentrated in vacuo. The mixture was purified by silica gel column (PE) to give the title compound (10.3 g, 98% yield) as yellow solid. ¹H NMR (400 MHz, DMSO-d₆) δ 7.90 (dd, J=6.4, 9.6 Hz, 1H), 6.78 (dd, J=7.6, 9.6 Hz, 2H), 2.76 (d, J=5.2 Hz, 3H), LC-MS (ESI⁺) m/z 248.9 (M+H)⁺.

### Step 2—3-Bromo-4-fluoro-N2-methyl-benzene-1,2-diamine

To a solution of 2-bromo-3-fluoro-N-methyl-6-nitro-aniline (10.0 g, 40.1 mmol) in THF (100 mL) was added Pt-V/C (524 mg, 2.01 mmol). The mixture was then stirred at 25° C. for 16 hrs under H₂ (15 psi). On completion, the mixture was filtered and concentrated in vacuo to give the title compound (8.7 g, 98% yield) as yellow solid. ¹H NMR (400 MHz, DMSO-d₆) δ 6.75-6.65 (m, 1H), 6.60 (dd, J=6.0, 8.8 Hz, 1H), 4.82 (s, 2H), 3.91 (s, 1H), 2.62 (d, J=4.0 Hz, 3H), LC-MS (ESI⁺) m/z 221.1 (M+H)⁺

### Step 3—4-Bromo-5-fluoro-3-methyl-1H-benzimidazol-2-one

To a solution of 3-bromo-4-fluoro-N2-methyl-benzene-1,2-diamine (8.70 g, 39.7 mmol) in ACN (120 mL) was added CDI (19.3 g, 119 mmol). The mixture was then stirred at 85° C. for 16 hrs. On completion, the mixture was concentrated in vacuo. The mixture was diluted with H₂O (300 mL), filtered and the filtrate was dried in vacuo to give the title compound (8.9 g, 91% yield) as gray solid, ¹H NMR (400 MHz, DMSO-d₆) δ 11.18 (s, 1H), 7.05-6.87 (m, 2H), 3.57 (s, 3H), LC-MS (ESI⁺) m/z 245.0 (M+H)⁺.

### Step 4—4-Bromo-5-fluoro-3-methyl-1-(2-trimethylsilylethoxymethyl)benzimidazol-2-one

A mixture of 4-bromo-5-fluoro-3-methyl-1H-benzimidazol-2-one (8.00 g, 32.6 mmol) in THF (150 mL) was degassed and purged with N₂ 3 times, and then the mixture was stirred at 0° C. for 30 min under N₂ atmosphere. Next, NaH (1.96 g, 48.9 mmol, 60% dispersion in mineral oil) was added in the mixture, which was degassed and purged with N₂ 3 times, and then the mixture was stirred at 0° C. for 1 h under N₂ atmosphere. Then SEM-Cl (8.16 g, 48.9 mmol) was added in the mixture, which was degassed and purged with N₂ for 3 times. Then the mixture was stirred at 65° C. for 11 hrs under N₂ atmosphere. On completion, the reaction mixture was quenched with water (100 mL), then the reaction mixture was concentrated under reduced pressure to remove THF, then the residue was diluted with water (100 mL) and extracted with ethyl acetate (200 mL×3). The combined organic layer was dried over Na₂SO₄, filtered and concentrated in vacuo to give a residue. The residue was purified by column chromatography (SiO₂, petroleum ether/ethyl acetate=10/0 to 2/1) to give the title compound (9.10 g, 72 yield) as a black brown oil. ¹H NMR (400 MHz, CDCl₃) δ 7.04 (dd, J=4.4, 8.8 Hz, 1H), 6.95-6.86 (m, 1H), 5.30 (s, 2H), 3.78 (s, 3H), 3.62-3.56 (m, 2H), 0.94-0.88 (m, 2H), 0.01-0.05 (m, 9H). LC-MS (ESI⁺) m/z 375.1 (M+H)⁺.

### Step 5—4-[4-(Dimethoxymethyl)-1-piperidyl]-5-fluoro-3-methyl-1-(2-trimethylsilylethoxymethyl) benzimidazol-2-one

A mixture of 4-bromo-5-fluoro-3-methyl-1-(2-trimethylsilylethoxymethyl)benzimidazol-2-one (200 mg, 532 μmol), 4-(dimethoxymethyl)piperidine (169 mg, 1.07 mmol, CAS #188646-83-5), XantPhos Pd G3 (50.5 mg, 53.2 mol), and Cs₂CO₃ (520 mg, 1.60 mmol) in dioxane (4 mL) was stirred at 110° C. for 16 hrs under N₂. On completion, the reaction was diluted with EA (20 mL). The organic layer was washed with water (20 ml), dried over Na₂SO₄ and concentrated in vacuo. The residue was purified by column chromatography (SiO₂, Petroleum ether/Ethyl acetate=10/1 to 2/1) to give the title compound (70 mg, 14% yield) as yellow oil. ¹H NMR (400 MHz, DMSO-d₆) δ 7.11-7.05 (m, 1H), 6.98-6.90 (m, 1H), 5.28 (s, 2H), 4.18 (d, J=6.4 Hz, 1H), 3.67 (s, 3H), 3.62-3.55 (m, 2H), 3.35 (s, 6H), 3.14-3.07 (m, 4H), 1.82-1.68 (m, 3H), 1.54-1.40 (m, 2H), 0.94-0.86 (m, 2H), 0.00 (s, 8H).

### Step 6—4-[4-(Dimethoxymethyl)-1-piperidyl]-5-fluoro-3-methyl-1H-benzimidazol-2-one

A mixture of 4-[4-(dimethoxymethyl)-1-piperidyl]-5-fluoro-3-methyl-1-(2-trimethylsilylethoxymethyl) benzimidazol-2-one (450 mg, 992 μmol) in TBAF (1 M, 15.0 mL) was stirred at 80° C. for 16 hrs. On completion, the reaction was diluted with EA (20 mL). The organic layer was washed with water (20 mL), dried over Na₂SO₄, filtered and concentrated in vacuo. The residue was purified by column chromatography (SiO₂, Petroleum ether/Ethyl acetate=3/1 to 1/1) to give the title compound (250 mg, 77% yield) as brown solid. LC-MS (ESI⁺) m/z 324.1 (M+H)⁺.

### Step 7—3-[4-[4-(dimethoxymethyl)-1-piperidyl]-5-fluoro-3-methyl-2-oxo-benzimidazol-1-yl]-1-[(4-methoxyphenyl)methyl]piperidine-2,6-dione

A solution of 4-[4-(dimethoxymethyl)-1-piperidyl]-5-fluoro-3-methyl-1H-benzimidazol-2-one (250 mg, 773 mol) and t-BuOK (130 mg, 1.16 mmol) in THF (6 mL) was stirred at 0° C. for 0.5 hr. Then, [1-[(4-methoxyphenyl)methyl]-2,6-dioxo-3-piperidyl] trifluoromethanesulfonate (442 mg, 1.16 mmol, Intermediate CY) was added at 0° C. and the mixture was stirred for 1 hr. On completion, the reaction was diluted with EA (20 mL). The organic layer was washed with water (20 mL), dried over Na₂SO₄ and concentrated in vacuo. The residue was purified by column chromatography (SiO₂, DCM/Ethyl acetate=5/1 to 1/1) to give the title compound (320 mg, 74% yield) as yellow solid. ¹H NMR (400 MHz, DMSO-d₆) δ 7.20 (d, J=8.4 Hz, 2H), 6.93-6.76 (m, 4H), 5.50 (dd, J=5.2, 13.2 Hz, 1H), 4.88-4.67 (m, 2H), 4.11 (d, J=6.4 Hz, 1H), 3.72 (s, 3H), 3.61 (s, 3H), 3.28 (s, 6H), 3.11-2.97 (m, 5H), 2.80 (d, J=17.6 Hz, 1H), 2.73-2.64 (m, 1H), 2.08-2.00 (m, 1H), 1.68 (d, J=10.0 Hz, 3H), 1.49-1.31 (m, 2H).

### Step 8—1-[5-Fluoro-1-[1-[(4-methoxyphenyl)methyl]-2,6-dioxo-3-piperidyl]-3-methyl-2-oxo-benzimidazol-4-yl]piperidine-4-carbaldehyde

A mixture of 3-[4-[4-(dimethoxymethyl)-1-piperidyl]-5-fluoro-3-methyl-2-oxo-benzimidazol-1-yl]-1-[(4-methoxyphenyl)methyl]piperidine-2,6-dione (250 mg, 450 μmol) in HCOOH (21.6 mg, 450 mol, 3 mL) was stirred at 80° C. for 1 hr. On completion, the reaction was concentrated in vacuo to give the title compound (249 mg, 99% yield, FA) as brown oil. LC-MS (ESI⁺) m/z 509.1 (M+H)⁺.

### Synthesis of 3-[4-[4-[[4-(3-Aminocyclobutyl)piperazin-1-yl]methyl]-1-piperidyl]-5-fluoro-3-methyl-2-oxo-benzimidazol-1-yl]piperidine-2,6-dione (Intermediate RH)

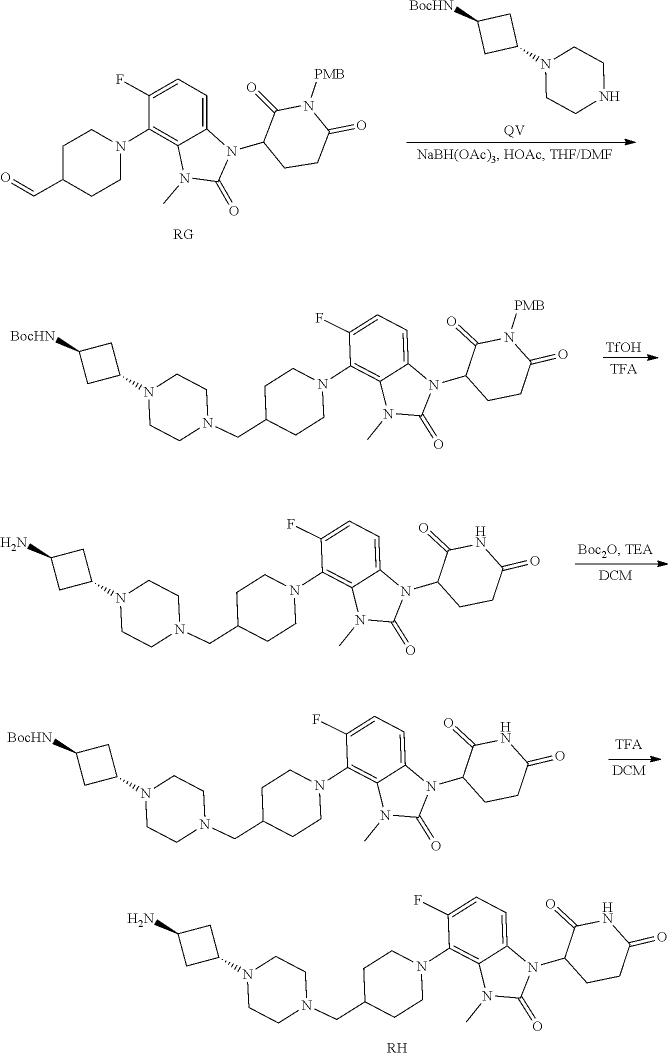

### Step 1—Tert-butyl N-[3-[4-[[1-[5-fluoro-1-[1-[(4-methoxyphenyl)methyl]-2,6-dioxo-3-piperidyl]-3-methyl-2-oxo-benzimidazol-4-yl]-4-piperidyl]methyl]piperazin-1-yl]cyclobutyl]carbamate

A solution of 1-[5-fluoro-1-[1-[(4-methoxyphenyl)methyl]-2,6-dioxo-3-piperidyl]-3-methyl-2-oxo-benzimidazol-4-yl]piperidine-4-carbaldehyde (249 mg, 449 μmol, Intermediate RG), HOAc (26.9 mg, 449 μmol, 25.7 μL) and tert-butyl N-(3-piperazin-1-ylcyclobutyl)carbamate (126 mg, 493 μmol, Intermediate QV) in DMF (1 mL) and THF (3 mL) was stirred at 25° C. for 0.5 hr. Then, NaBH(OAc)₃ (142 mg, 673 mol) was added and the mixture was stirred at 25° C. for 1 hr. On completion, the reaction was quenched with water (0.1 mL) and concentrated in vacuo. The residue was purified by prep-HPLC (column: Welch Ultimate C18 150*25 mm*5 um; mobile phase: [water (FA)-ACN]; gradient: 20%-50% B over 11 min) to give the title compound (220 mg, 65% yield) as white solid. ¹H NMR (400 MHz, DMSO-d₆) δ 7.28-7.11 (m, 3H), 6.92-6.73 (m, 4H), 5.51 (dd, J=5.2, 12.8 Hz, 1H), 4.88-4.68 (m, 2H), 3.93-3.82 (m, 1H), 3.73 (s, 3H), 3.61 (s, 3H), 3.12-3.00 (m, 5H), 2.86-2.66 (m, 4H), 2.43-2.23 (m, 6H), 2.18 (d, J=7.2 Hz, 2H), 2.14-1.99 (m, 4H), 1.94-1.86 (m, 2H), 1.79-1.68 (m, 2H), 1.68-1.56 (m, 1H), 1.37 (s, 9H), 1.31-1.18 (m, 2H).

### Step 2—3-[4-[4-[[4-(3-Aminocyclobutyl)piperazin-1-yl]methyl]-1-piperidyl]-5-fluoro-3-methyl-2-oxo-benzimidazol-1-yl]piperidine-2,6-dione

A mixture of tert-butyl N-[3-[4-[[1-[5-fluoro-1-[1-[(4-methoxyphenyl)methyl]-2,6-dioxo-3-piperidyl]-3-methyl-2-oxo-benzimidazol-4-yl]-4-piperidyl]methyl]piperazin-1-yl]cyclobutyl]carbamate (130 mg, 173 mol) in TfOH (734 mg, 4.90 mmol, 433 μL) and TFA (2.00 g, 17.5 mmol, 1.30 mL) was stirred at 70° C. for 1 hr. On completion, the reaction was concentrated in vacuo to give the title compound (111 mg, 99% yield, TFA) as brown oil. LC-MS (ESI⁺) m/z 528.2 (M+H)⁺.

### Step 3—Tert-butyl N-[3-[4-[[1-[1-(2,6-dioxo-3-piperidyl)-5-fluoro-3-methyl-2-oxo-benzimidazol-4-yl]-4-piperidyl]methyl]piperazin-1-yl]cyclobutyl]carbamate

To a solution of 3-[4-[4-[[4-(3-aminocyclobutyl)piperazin-1-yl]methyl]-1-piperidyl]-5-fluoro-3-ethyl-2-oxo-benzimidazol-1-yl]piperidine-2,6-dione (111 mg, 172 μmol, TFA) and TEA (52.5 mg, 518 μmol, 72.2 μL) in DCM (2 mL) was added Boc₂O (41.5 mg, 190 μmol). The reaction was then stirred at 25° C. for 1 hr. On completion, the reaction was diluted with DCM (10 mL). The organic layer was washed with water (10 mL×3), dried over Na₂SO₄ and concentrated in vacuo. The crude product was triturated with PE:EA=5:1 (5 mL) at 25 C for 30 min. The mixture was filtered and the filtered cake was dried in vacuo to give the title compound (108 mg, 99% yield) as brown solid. LC-MS (ESI⁺) m/z 628.3 (M+H)⁺.

### Step 4—3-[4-[4-[[4-(3-Aminocyclobutyl)piperazin-1-yl]methyl]-1-piperidyl]-5-fluoro-3-methyl-2-oxo -benzimidazol-1-yl]piperidine-2,6-dione

A mixture of tert-butyl N-[3-[4-[[1-[1-(2,6-dioxo-3-piperidyl)-5-fluoro-3-methyl-2-oxo -benzimidazol-4-yl]-4-piperidyl]methyl]piperazin-1-yl]cyclobutyl]carbamate (108 mg, 172 μmol) and TFA (460 mg, 4.04 mmol, 0.3 mL) in DCM (1 mL) was stirred at 25° C. for 1 hr. On completion, the reaction mixture was concentrated in vacuo to give the title compound (110 mg, 99% yield, TFA) as yellow oil. LC-MS (ESI⁺) m/z 528.2 (M+H)⁺.

### Synthesis of 1-[5-Chloro-1-(2,6-dioxo-3-piperidyl)-3-methyl-2-oxo-benzimidazol-4-yl]piperidine-4-carbaldehyde (Intermediate RI)

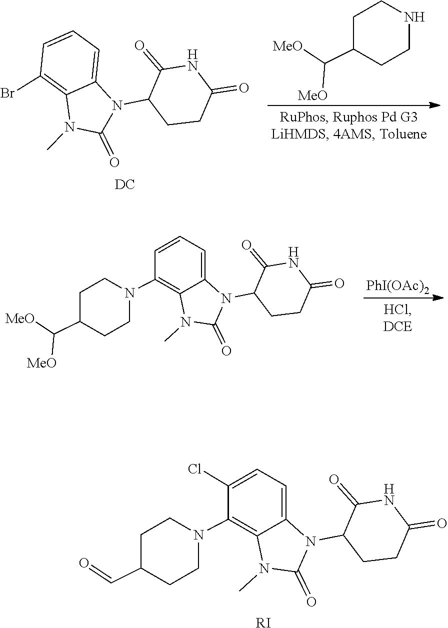

### Step 1—3-[4-[4-(Dimethoxymethyl)-1-piperidyl]-3-methyl-2-oxo-benzimidazol-1-yl]piperidine-2,6-dione

To a solution of 3-(4-bromo-3-methyl-2-oxo-benzimidazol-1-yl)piperidine-2,6-dione (10.0 g, 29.5 mmol, Intermediate DC) and 4-(dimethoxymethyl)piperidine (5.65 g, 35.4 mmol, CAS #188646-83-5) in toluene (200 mL) was added RuPhos (2.07 g, 4.44 mmol), RuPhos Pd G3 (3.71 g, 4.44 mmol), 4A molecular sieves (200 mg) and LiHMDS (1 M, 103 mL). Then the mixture was purged with N₂ for three times and stirred at 110° C. for 4 hrs. On completion, the mixture was added FAto pH=6, then filtered and concentrated in vacuo to give a residue. The residue was purified by column chromatography (SiO₂, PE:EA=10:1 to 0:1) to give the title compound (7.50 g, 60% yield) as a brown solid. ¹H NMR (400 MHz, DMSO-d₆) δ 11.09 (s, 1H), 6.99-6.93 (m, 1H), 6.91-6.83 (m, 2H), 5.40-5.31 (m, 1H), 4.13 (d, J=5.6 Hz, 1H), 3.61 (s, 3H), 3.29 (s, 6H), 3.11 (d, J=11.2 Hz, 2H), 2.91-2.84 (m, 1H), 2.72-2.58 (m, 4H), 2.02-1.97 (m, 1H), 1.77-1.65 (m, 3H), 1.51-1.39 (m, 2H). LC-MS (ESI⁺) m/z 417.2 (M+H)⁺.

### Step 2—1-[5-Chloro-1-(2,6-dioxo-3-piperidyl)-3-methyl-2-oxo-benzimidazol-4-yl]piperidine-4-carbaldehyde

To a solution of 3-[4-[4-(dimethoxymethyl)-1-piperidyl]-3-methyl-2-oxo-benzimidazol-1-yl]piperidine-2,6-dione (500 mg, 1.20 mmol) in DCE (20 mL) was added PhI(OAc)₂ (386 mg, 1.20 mmol) and HCl (1 M, 6.00 mL), then the mixture was stirred at 50° C. for 3 hrs. On completion, the mixture was separated to give the organic layer, then concentrated in vacuo to give a residue. The residue was purified by prep-HPLC (column: Phenomenex Luna C18 200*40 mm*10 um; mobile phase: [water (TFA)-ACN]; gradient: 28%-58% B over 10 min) to give the title compound (85 mg, 17% yield) as a white solid. ¹H NMR (400 MHz, CDCl₃) δ 9.75 (s, 1H), 8.11 (s, 1H), 7.01 (d, J=8.4 Hz, 1H), 6.58 (d, J=8.4 Hz, 1H), 5.21 (dd, J=5.4, 12.8 Hz, 1H), 3.72 (s, 3H), 3.71-3.63 (m, 2H), 3.14-3.05 (m, 2H), 3.00-2.92 (m, 1H), 2.89-2.79 (m, 1H), 2.74-2.68 (m, 1H), 2.48-2.40 (m, 1H), 2.28-2.20 (m, 1H), 2.06-1.99 (m, 2H), 1.76-1.64 (m, 2H). LC-MS (ESI⁺) m/z 405.1 (M+H)⁺.

### Synthesis of Benzyl N-[3-[(1S,4S)-2,5-diazabicyclo[2.2.1]heptan-2-yl]cyclobutyl]carbamate (Intermediate RJ)

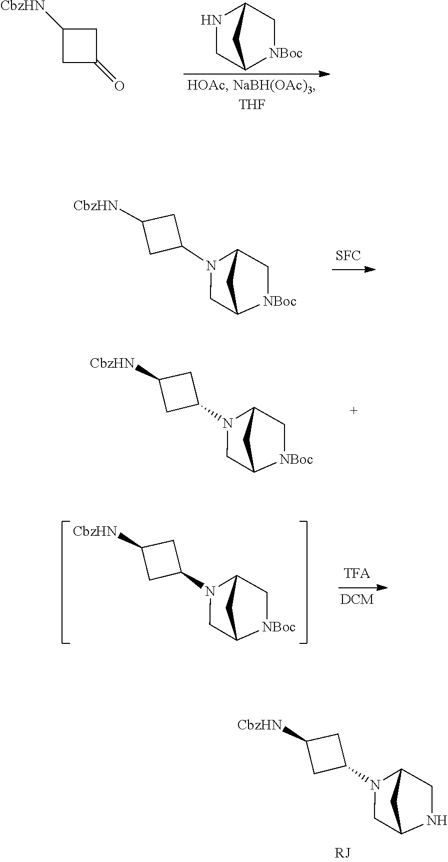

### Step 1—Tert-butyl (1S,4S)-5-[3-(benzyloxycarbonylamino)cyclobutyl]-2,5-diazabicyclo[2.2.1]heptane-2-carboxylate

To a mixture of benzyl N-(3-oxocyclobutyl)carbamate (5.00 g, 22.8 mmol, CAS #130369-36-7) in THF (50 mL) was added HOAc (1.37 g, 22.8 mmol) and tert-butyl (1S,4S) -2,5-diazabicyclo[2.2.1]heptane-2-carboxylate (4.52 g, 22.8 mmol, CAS #113451-59-5). The reaction mixture was stirred at 25° C. for 0.5 hr, then the NaBH(OAc)₃ (9.67 g, 45.6 mmol) was added. The reaction mixture was then stirred at 25° C. for 1 hr. On completion, the reaction mixture was quenched with water (0.5 mL) and concentrated in vacuo. The residue was purified by reverse phase (0.1% FA condition) to give the title compound (3.00 g, 32% yield) as white solid. LC-MS (ESI⁺) m/z 402.1 (M+H)⁺.

### Step 2—Tert-butyl (1S,4S)-5-((1r,3S)-3-(((benzyloxy)carbonyl)amino)cyclobutyl)-2,5-diazabicyclo[2.2.1]heptane-2-carboxylate and tert-butyl (1S,4S)-5-((1s,3R)-3-(((benzyloxy)carbonyl)amino)cyclobutyl)-2,5-diazabicyclo[2.2.1]heptane-2-carboxylate

Tert-butyl (1S,4S)-5-[3-(benzyloxycarbonylamino)cyclobutyl]-2,5-diazabicyclo[2.2.1]heptane-2-carboxylate was separated by SFC (column: DAICEL CHIRALPAK AS (250 mm*50 mm, 10 um); mobile phase: [CO₂-i-PrOH (0.1% NH₃H₂O)]; B %:30%, isocratic elution mode) to give tert-butyl (1S,4S)-5-((1r,3S)-3-(((benzyloxy)carbonyl)amino)cyclobutyl)-2,5-diazabicyclo[2.2.1]heptane-2-carboxylate (980 mg, 32% yield, peak 1, trans) as white solid (¹H NMR (400 MHz, DMSO-d₆) δ 7.53 (s, 1H), 7.39-7.29 (m, 5H), 4.98 (s, 2H), 4.16 (d, J=11.2 Hz, 1H), 3.75-3.62 (m, 1H), 3.33 (d, J=0.8 Hz, 2H), 3.23 (d, J=1.6 Hz, 1H), 3.06 (s, 1H), 2.95-2.79 (m, 1H), 2.76-2.64 (m, 1H), 2.40-2.24 (m, 2H), 1.87-1.54 (m, 4H), 1.39 (s, 9H)) and tert-butyl (1S,4S)-5-((1s,3R)-3-(((benzyloxy)carbonyl)amino)cyclobutyl)-2,5-diazabicyclo[2.2.1]heptane-2-carboxylate (1.20 g, 40% yield, peak 2, cis) as white solid (¹H NMR (400 MHz, DMSO-d₆) δ 7.61 (d, J=7.6 Hz, 1H), 7.38-7.28 (m, 5H), 4.99 (s, 2H), 4.18 (d, J=15.2 Hz, 1H), 4.14-4.07 (m, 1H), 3.61 (s, 1H), 3.35-3.21 (m, 2H), 3.14-3.05 (m, 1H), 2.77-2.70 (m, 1H), 2.68-2.58 (m, 1H), 2.21-2.11 (m, 2H), 2.05-1.95 (m, 2H), 1.81-1.72 (m, 1H), 1.71-1.63 (m, 1H), 1.39 (s, 9H)). The absolute stereochemistry of the diastereomers was assigned by 2D NMR.

### Step 3—Benzyl N-[3-[(1S,4S)-2,5-diazabicyclo[2.2.1]heptan-2-yl]cyclobutyl]carbamate

A solution of tert-butyl(1S,4S)-5-[3-(benzyloxycarbonylamino)cyclobutyl]-2,5-diazabicyclo[2.2.1] heptane-2-carboxylate (300 mg, 747 μmol) and TFA (1.54 g, 13.4 mmol) in DCM (2 mL) was stirred at 25° C. for 0.5 hr. On completion, the reaction mixture was concentrated in vacuo to give the title compound (310 mg, 99% yield, TFA) as white oil. LC-MS (ESI⁺) m/z 302.1 (M+H)⁺.

### Synthesis of 3-[4-[4-[[(1S,4S)-5-(3-aminocyclobutyl)-2,5-diazabicyclo[2.2.1]heptan-2-yl]methyl]-1-piperidyl]-5-chloro-3-methyl-2-oxo-benzimidazol-1-yl]piperidine-2,6-dione (Intermediate RJ)

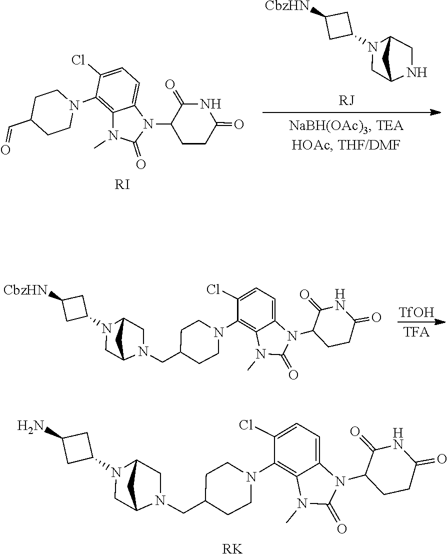

### Step 1—Benzyl N-[3-[(1S,4S)-5-[[1-[5-chloro-1-(2,6-dioxo-3-piperidyl)-3-methyl-2-oxo-benzimidazol-4-yl]-4-piperidyl]methyl]-2,5-diazabicyclo[2.2.1]heptan-2-yl]cyclobutyl]carbamate

To a solution of benzyl N-[3-[(1S,4S)-2,5-diazabicyclo[2.2.1]heptan-2-yl]cyclobutyl]carbamate (210 mg, 505 mol, TFA, Intermediate RJ) in THF (2 mL) and DMF (2 mL) was added TEA (1.44 mmol, 0.2 mL), then 1-[5-chloro-1-(2,6-dioxo-3-piperidyl)-3-methyl-2-oxo-benzimidazol-4-yl]piperidine-4-carbaldehyde (204 mg, 505 mol, Intermediate RI) and AcOH (1.75 mmol, 0.1 mL) was added and the mixture was stirred at 25° C. for 0.2 hr. Next, NaBH(OAc)₃ (160 mg, 758 μmol) was added and the mixture was stirred 25° C. for 1 hr. On completion, the mixture was filtered to give the residue. The residue was purified by prep-HPLC (column: Phenomenex Luna C18 150*25 mm*10 um; mobile phase: [water (FA)-ACN]; gradient: 11%-41% B over 10 min) to give the title compound (210 mg, 60% yield) as a colorless liquid. LC-MS (ESI⁺) m/z 690.2 (M+H)⁺.

### Step 2—3-[4-[4-[[(1S,4S)-5-(3-aminocyclobutyl)-2,5-diazabicyclo[2.2.1]heptan-2-yl]methyl]-1-piperidyl]-5-chloro-3-methyl-2-oxo-benzimidazol-1-yl]piperidine-2,6-dione

To a solution of benzyl N-[3-[(1S,4S)-5-[[1-[5-chloro-1-(2,6-dioxo-3-piperidyl)-3-methyl-2-oxo-benzimidazol-4-yl]-4-piperidyl]methyl]-2,5-diazabicyclo[2.2.1]heptan-2-yl]cyclobutyl]carbamate (110 mg, 159 μmol) in TFA (2 mL) was added TfOH (7.91 mmol, 0.7 mL), then the mixture was stirred at 70° C. for 1 hr. On completion, the mixture was concentrated in vacuo to give the title compound (100 mg, 93% yield, TFA) as a brown oil. LC-MS (ESI⁺) m/z 556.3 (M+H)⁺.

### Synthesis of 3-[4-[4-[(4-Aminocyclohexyl)methyl]piperazin-1-yl]phenyl]piperidine-2,6-dione (Intermediate RL)

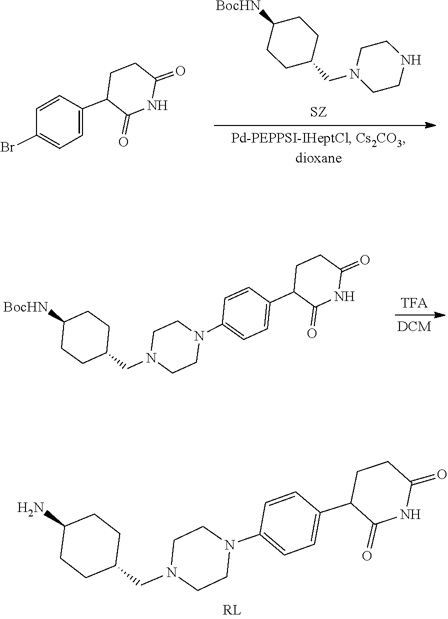 [SMILES: O=C1CCC(c2ccc(Br)cc2)C(=O)N1 ; CC(C)(C)OC(=O)N[C@H]1CC[C@H](CN2CCNCC2)CC1 ; CC(C)(C)OC(=O)N[C@H]1CC[C@H](CN2CCN(c3ccc(C4CCC(=O)NC4=O)cc3)CC2)CC1 ; N[C@H]1CC[C@H](CN2CCN(c3ccc(C4CCC(=O)NC4=O)cc3)CC2)CC1]

### Step 1—Tert-butyl N-[4-[[4-[4-(2,6-dioxo-3-piperidyl)phenyl]piperazin-1-yl]methyl]cyclohexyl]carbamate

To a solution of 3-(4-bromophenyl)piperidine-2,6-dione (500 mg, 1.86 mmol, CAS #1267337-47-2) and tert-butyl N-[4-(piperazin-1-ylmethyl)cyclohexyl]carbamate (554 mg, 1.86 mmol, Intermediate SZ) in dioxane (10 mL) was added Cs₂CO₃ (1.22 g, 3.73 mmol) and Pd-PEPPSI-IHeptCl (182 mg, 186 mol). Then the mixture was stirred at 100° C. for 2 hrs under N₂. On completion, the reaction mixture diluted with water (5 mL) and extracted with EA (20 mL×2). The combined organic layers were washed with water (5 mL×2), dried over Na₂SO₄, filtered and concentrated under reduced pressure. The residue was purified by prep-HPLC (column: Phenomenex Luna C18 150*25 mm*10 um; mobile phase: [water (FA)-ACN]; gradient: 7%-37% B over 10 min) to give the title compound (105 mg, 11% yield) as white solid. ¹H NMR (400 MHz, DMSO-d₆) δ 10.78 (s, 1H), 7.04 (d, J=8.4 Hz, 2H), 6.87 (d, J=8.4 Hz, 2H), 6.71 (d, J=7.8 Hz, 1H), 3.72 (dd, J=4.8, 11.0 Hz, 2H), 3.09 (s, 6H), 2.46 (d, J=3.6 Hz, 4H), 2.12 (d, J=7.6 Hz, 2H), 2.07 (s, 4H), 1.76 (d, J=10.0 Hz, 3H), 1.37 (s, 9H), 1.12 (q, J=11.2 Hz, 2H), 0.95-0.77 (m, 2H).

### Step 2—3-[4-[4-[(4-Aminocyclohexyl)methyl]piperazin-1-yl]phenyl]piperidine-2,6-dione

To a solution of tert-butyl N-[4-[[4-[4-(2,6-dioxo-3-piperidyl)phenyl]piperazin-1-yl]methyl]cyclohexyl]carbamate (100 mg, 206 μmol) in DCM (3 mL) was added TFA (1 mL), then the mixture was stirred at 25° C. for 1 hr. On completion, the reaction was concentrated in vacuo to give the title compound (100 mg, 97% yield, TFA) as yellow oil. LC-MS (ESI⁺) m/z 385.1 (M+H)⁺.

### Synthesis of 3-(4-bromo-2-chloro-5-fluoro-phenyl)piperidine-2,6-dione (Intermediate RM)

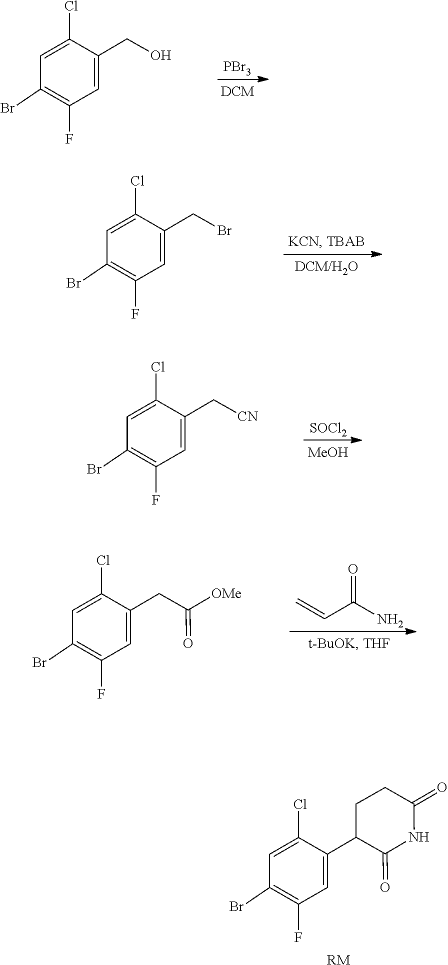

### Step 1—1-Bromo-4-(bromomethyl)-5-chloro-2-fluoro-benzene

To a solution of (4-bromo-2-chloro-5-fluoro-phenyl)methanol (5 g, 20.8 mmol, CAS #1338254-21-9) in DCM (50 mL) was added PBr₃ (8.48 g, 31.3 mmol) at 0° C. and the mixture was stirred at 0° C. for 1 hr. Then, the mixture was warmed to rt and stirred for 16 hrs. On completion, the mixture was added into NaHCO₃ (80 mL) aqueous solution slowly to quench the reaction. Then the mixture was diluted with DCM (50 mL) and washed with water (20 mL×3). The organic layer was dried over Na₂SO₄, filtered and concentrated in vacuo to give the title compound (3.5 g, 55% yield) as colorless oil. ¹H NMR (400 MHz, DMSO-d₆) δ 7.97 (d, J=6.4 Hz, 1H), 7.74 (d, J=9.2 Hz, 1H), 4.69 (s, 2H).

### Step 2—2-(4-Bromo-2-chloro-5-fluoro-phenyl)acetonitrile

To a solution of 1-bromo-4-(bromomethyl)-5-chloro-2-fluoro-benzene (3.5 g, 11.5 mmol) and TBAB (447 mg, 1.39 mmol) in DCM (20 mL) and H₂O (20 mL) was added KCN (2.25 g, 34.5 mmol) slowly. The reaction mixture was stirred at rt for 16 hrs. On completion, the reaction mixture was added into ice water (100 mL). Then, the mixture was extracted with DCM (100 mL×2), the combined organic layers were dried over Na₂SO₄, filtered and concentrated in vacuo. The residue was purified by column chromatography (SiO₂, PE:EA=20:1 to 10:1) to give the title compound (2.3 g, 79% yield) as colorless oil. ¹H NMR (400 MHz, DMSO-d₆) δ 8.02 (d, J=6.4 Hz, 1H), 7.60 (d, J=9.2 Hz, 1H), 4.09 (s, 2H).

### Step 3—Methyl 2-(4-bromo-2-chloro-5-fluoro-phenyl)acetate

To a solution of 2-(4-bromo-2-chloro-5-fluoro-phenyl)acetonitrile (2.3 g, 9.26 mmol) in MeOH (17 mL) was added dropwise SOCl₂ (12 mL) at 0° C. The reaction mixture was stirred at rt for 16 hrs under N₂. On completion, the mixture was concentrated in vacuo. Then, the crude product was diluted with EA (20 mL) and washed with water (15 mL). The organic layer was dried over Na₂SO₄, filtered and concentrated in vacuo to give the title compound (2.7 g, 89% yield) as yellow solid. ¹H NMR (400 MHz, DMSO-d₆) δ 7.91 (d, J=6.4 Hz, 1H), 7.54 (d, J=9.2 Hz, 1H), 3.83 (s, 2H), 3.63 (s, 3H).

### Step 4—3-(4-Bromo-2-chloro-5-fluoro-phenyl)piperidine-2,6-dione

To a solution of methyl 2-(4-bromo-2-chloro-5-fluoro-phenyl)acetate (1 g, 3.55 mmol) and prop-2-enamide (504 mg, 7.10 mmol) in THF (10 mL) was added t-BuOK (597 mg, 5.33 mmol). The reaction mixture was stirred at rt for 0.5 hr. On completion, the reaction mixture was added NH₄Cl aqueous solution (5 mL) and diluted with water (30 mL). Then, the mixture was extracted with EA (50 mL×2). The combined organic layers were dried over Na₂SO₄, filtered and concentrated in vacuo. The crude product was triturated with DCM (20 mL) at rt for 5 min, then filtered to give the title compound (1.2 g, 52% yield) as white solid. ¹H NMR (400 MHz, DMSO-d₆) δ 10.96 (s, 1H), 7.98-7.85 (m, 1H), 7.53-7.46 (m, 1H), 4.24 (dd, J=4.8, 12.8 Hz, 1H), 2.86-2.73 (m, 1H), 2.57 (dd, J=2.4, 4.0 Hz, 1H), 2.39-2.28 (m, 1H), 2.01-1.94 (m, 1H).

### Synthesis of 3-[4-[4-[(4-Aminocyclohexyl)methyl]piperazin-1-yl]-2-chloro-5-fluoro-phenyl]piperidine-2,6-dione (Intermediate RN)

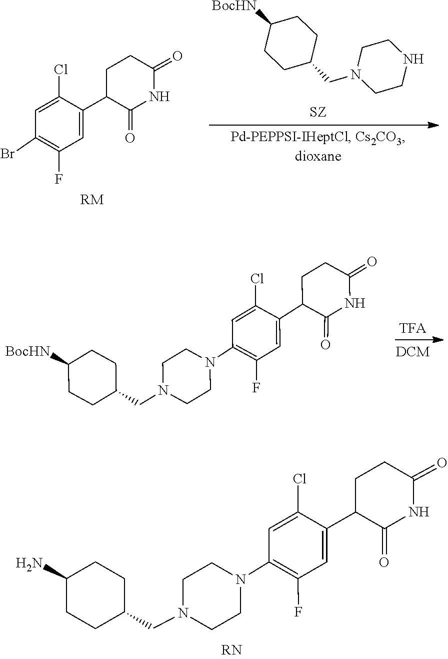

### Step 1—Tert-butyl N-[4-[[4-[5-chloro-4-(2,6-dioxo-3-piperidyl)-2-fluoro-phenyl]piperazin-1-yl]methyl]cyclohexyl]carbamate

To a solution of 3-(4-bromo-2-chloro-5-fluoro-phenyl)piperidine-2,6-dione (600 mg, 1.87 mmol, Intermediate RM) and tert-butyl N-[4-(piperazin-1-ylmethyl)cyclohexyl]carbamate (835 mg, 2.81 mmol, Intermediate SZ) in dioxane (12 mL) was added Cs₂CO₃ (1.83 g, 5.62 mmol) and 1,3-bis[2,6-bis(1-propylbutyl)phenyl]-4,5-dichloro-2H-imidazol-1-ium-2-ide; 3-chloropyridine dichloropalladium (182 mg, 187 mol). The reaction mixture was then stirred at 100° C. for 4 hrs under N₂. On completion, the reaction mixture was diluted with EA (50 mL) and washed with water (30 mL×2). The organic layer was dried over Na₂SO₄, filtered and concentrated in vacuo. The residue was purified by column chromatography (SiO₂, PE:EA=10:1 to 0:1) to give the title compound (85 mg, 8% yield) as brown solid. ¹H NMR (400 MHz, DMSO-d₆) δ 10.86 (s, 1H), 7.17 (d, J=13.6 Hz, 1H), 7.01 (d, J=8.4 Hz, 1H), 6.69 (d, J=7.6 Hz, 1H), 4.10 (dd, J=4.8, 12.8 Hz, 1H), 3.19-3.09 (m, 1H), 3.02 (s, 4H), 2.81-2.70 (m, 1H), 2.46 (s, 3H), 2.33-2.25 (m, 1H), 2.11 (d, J=7.2 Hz, 2H), 1.96-1.89 (m, 1H), 1.76 (d, J=11.2 Hz, 4H), 1.37 (s, 9H), 1.29-1.05 (m, 4H), 0.92-0.79 (m, 3H).

### Step 2—3-[4-[4-[(4-Aminocyclohexyl)methyl]piperazin-1-yl]-2-chloro-5-fluoro-phenyl]piperidine-2,6-dione

A mixture of tert-butyl N-[4-[[4-[5-chloro-4-(2,6-dioxo-3-piperidyl)-2-fluoro-phenyl]piperazin-1-yl] methyl]cyclohexyl]carbamate (75 mg, 139 μmol) in TFA (0.5 mL) and DCM (1.5 mL) was stirred at rt for 0.5 hr. On completion, the mixture was concentrated in vacuo to give the title compound (76 mg, 98% yield, TFA) as brown oil. LC-MS (ESI⁺) m/z 437.1 (M+H)⁺.

### Synthesis of 1-[3-chloro-4-(2,6-dioxo-3-piperidyl)phenyl]piperidine-4-carbaldehyde (Intermediate RO)

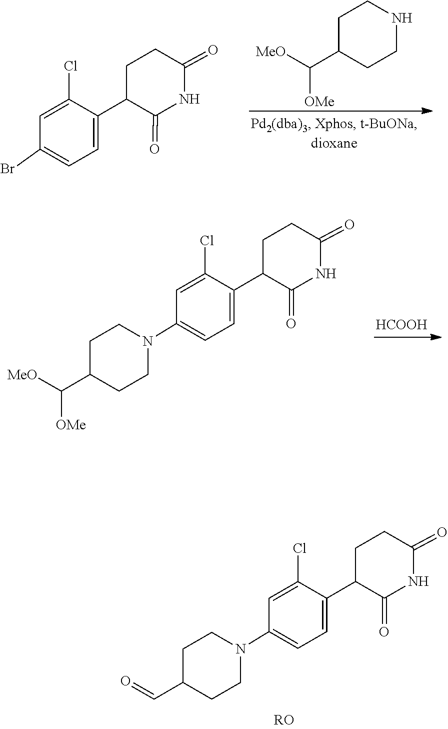 [SMILES: O=C1CCC(c2ccc(Br)cc2Cl)C(=O)N1 ; COC(OC)C1CCNCC1 ; COC(OC)C1CCN(c2ccc(C3CCC(=O)NC3=O)c(Cl)c2)CC1 ; "HCOOH" ; O=CC1CCN(c2ccc(C3CCC(=O)NC3=O)c(Cl)c2)CC1]

### Step 1—3-[2-Chloro-4-[4-(dimethoxymethyl)-1-piperidyl]phenyl]piperidine-2,6-dione

To a solution of 3-(4-bromo-2-chloro-phenyl)piperidine-2,6-dione (1.00 g, 3.31 mmol, synthesized via Steps 1-2 of Intermediate PH), 4-(dimethoxymethyl)piperidine (789 mg, 4.96 mmol, CAS #188646-83-5) in dioxane (15 mL) was added tBuONa (953 mg, 9.92 mmol), Pd₂(dba)₃ (303 mg, 331 μmol) and XPhos (158 mg, 331 μmol). Then the mixture was stirred at 100° C. for 2 hrs. On completion, the mixture was concentrated in vacuo to give the residue. The residue was purified by column chromatography (SiO₂, PE:EA=30:1 to 20:1) to give the title compound (400 mg, 32% yield) as a yellow oil. ¹H NMR (400 MHz, DMSO-d₆) δ 10.84 (s, 1H), 7.09 (d, J=8.4 Hz, 1H), 6.92 (d, J=2.4 Hz, 1H), 6.89-6.84 (m, 1H), 4.09-4.00 (m, 2H), 3.73-3.71 (m, 2H), 3.26 (s, 6H), 2.82-2.58 (m, 4H), 2.27-2.16 (m, 1H), 1.97-1.88 (m, 1H), 1.70-1.67 (m, 2H), 1.34-1.22 (m, 3H). LC-MS (ESI⁺) m/z 381.0 (M+H)⁺.

### Step 2—1-[3-Chloro-4-(2,6-dioxo-3-piperidyl)phenyl]piperidine-4-carbaldehyde

A solution of 3-[2-chloro-4-[4-(dimethoxymethyl)-1-piperidyl]phenyl]piperidine-2,6-dione (200 mg, 525 μmol) in HCOOH (2 mL) was stirred at 80° C. for 0.5 hr. On completion, the mixture was concentrated in vacuo to give the title compound (170 mg, 97% yield) as a brown oil. LC-MS (ESI⁺) m z 352.8 (M+18)+.

### Synthesis of 4-[[4-[(3S)-3-hydroxy-3-methyl-1-piperidyl]-5-(trifluoromethyl)pyrimidin-2-yl]amino]-3-methyl-N-(4-methyl-4-piperidyl)benzenesulfonamide (Intermediate RP)

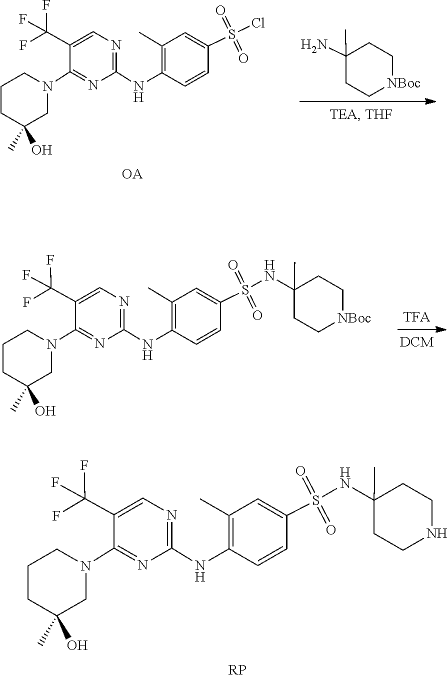 [SMILES: Cc1cc(S(=O)(=O)Cl)ccc1Nc1ncc(C(F)(F)F)c(N2CCC[C@](C)(O)C2)n1 ; CC1(N)CCN(C(=O)OC(C)(C)C)CC1 ; Cc1cc(S(=O)(=O)NC2(C)CCN(C(=O)OC(C)(C)C)CC2)ccc1Nc1ncc(C(F)(F)F)c(N2CCC[C@](C)(O)C2)n1 ; Cc1cc(S(=O)(=O)NC2(C)CCNCC2)ccc1Nc1ncc(C(F)(F)F)c(N2CCC[C@](C)(O)C2)n1]

### Step 1—Tert-butyl 4-[[4-[[4-[(3S)-3-hydroxy-3-methyl-1-piperidyl]-5-(trifluoromethyl)pyrimidin-2-yl]amino]-3-methyl-phenyl] sulfonylamino]-4-methyl-piperidine-1-carboxylate

To a solution of tert-butyl 4-amino-4-methyl-piperidine-1l-carboxylate (184 mg, 860 μmol, CAS#343788-69-2) and TEA (130 mg, 1.29 mmol) in THF (4 mL) was added 4-[[4-[(3S)-3-hydroxy-3-methyl-1-piperidyl]-5-(trifluoromethyl)pyrimidin-2-yl]amino]-3-methyl-benzenesulfonyl chloride (200 mg, 430 mol, Intermediate OA) at 50° C., then the mixture was stirred at 50° C. for 18 hrs. On completion, the reaction was concentrated in vacuo. The residue was purified by column chromatography (SiO₂, EA in PE, 32% to 45%) to give the title compound (250 mg, 90% yield) as white solid. ¹H NMR (400 MHz, DMSO-d₆) δ 9.09 (s, 1H), 8.34 (s, 1H), 7.86-7.78 (m, 1H), 7.70-7.58 (m, 2H), 7.35 (s, 1H), 4.44 (s, 1H), 3.63-3.54 (m, 1H), 3.44-3.33 (m, 3H), 3.28-3.18 (m, 2H), 3.17-3.03 (m, 2H), 2.32 (s, 3H), 1.82-1.70 (m, 3H), 1.61-1.49 (m, 2H), 1.36 (s, 10H), 1.33-1.25 (m, 2H), 1.03 (d, J=3.2 Hz, 6H); LC-MS (ESI⁺) m/z 643.1 (M+H)⁺.

### Step 2—4-[[4-[(3S)-3-hydroxy-3-methyl-1-piperidyl]-5-(trifluoromethyl)pyrimidin-2-yl]amino]-3-methyl-N-(4-methyl-4-piperidyl)benzenesulfonamide

A mixture of tert-butyl 4-[[4-[[4-[(3S)-3-hydroxy-3-methyl-1-piperidyl]-5-(trifluoromethyl)pyrimidin-2-yl]amino]-3-methyl-phenyl]sulfonylamino]-4-methyl-piperidine-1-carboxylate (85 mg, 132 mol) in DCM (0.9 mL) and TFA (460 mg, 4.04 mmol, 0.3 mL) was stirred at rt for 0.5 hr. On completion, the reaction mixture was concentrated in vacuo to give the title compound (86 mg, 99% yield, TFA) as colorless oil. LC-MS (ESI⁺) m/z 543.1 (M+H)⁺.

### Synthesis of 3-(4-Piperazin-1-ylphenyl)piperidine-2,6-dione (Intermediate RQ)

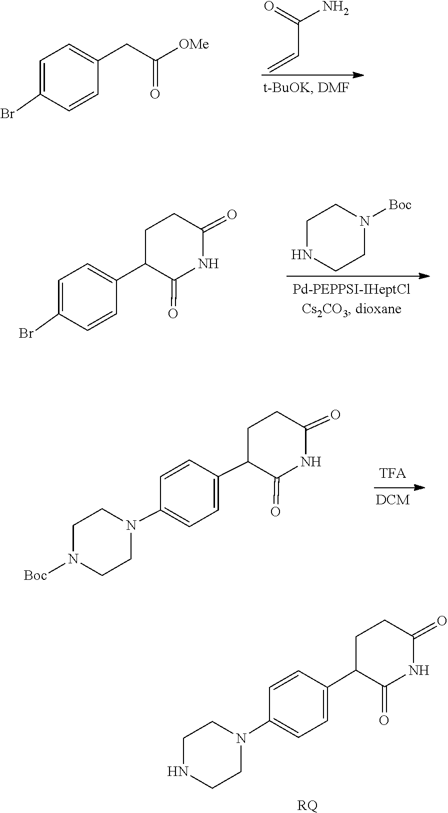

### Step 1—3-(4-Bromophenyl)piperidine-2,6-dione

To a solution of methyl 2-(4-bromophenyl) acetate (5 g, 21.8 mmol, CAS #41841-16-1) and prop-2-enamide (1.55 g, 21.8 mmol, CAS #79-06-1) in DMF (30 mL) was added t-BuOK (7.35 g, 65.4 mmol), the mixture was then stirred at 25° C. for 3 hrs under N₂. On completion, the reaction mixture was diluted with water (50 mL) and extracted with EA (100 mL×2). The combined organic layers were washed with water (50 mL×2), dried over Na₂SO₄, filtered and concentrated under reduced pressure to give the residue. The residue was purified by column chromatography (Silica gel, EA in PE, 30%, v/v) to give the title compound (2 g, 34% yield) as white solid. LC-MS (ESI⁺) m/z 267.8 (M+H)⁺.

### Step 2—Tert-butyl 4-[4-(2,6-dioxo-3-piperidyl)phenyl]piperazine-1-carboxylate

To a solution of 3-(4-bromophenyl)piperidine-2,6-dione (900 mg, 3.36 mmol) and tert-butyl piperazine-1-carboxylate (813 mg, 4.36 mmol, CAS #57260-71-6) in dioxane (10 mL) was added 1,3-bis[2,6-bis(1-propylbutyl)phenyl]-4,5-dichloro-2H-imidazol-1-ium-2-ide; 3-chloropyridine; dichloropalladium (326 mg, 335 μmol) and Cs₂CO₃ (3.28 g, 10.0 mmol). Then the mixture was stirred at 90° C. for 2 hrs under N₂. On completion, the reaction mixture was diluted with EA (30 mL), filtered, and the filtrate was concentrated under reduced pressure to give a residue. The residue was purified by column chromatography (Silica gel, EA in PE, 30%, v/v) to give the title compound (240 mg, 19% yield) as white solid. ¹H NMR (400 MHz, DMSO-d₆) δ 10.78 (s, 1H), 7.10-7.04 (m, 2H), 6.91 (d, J=8.8 Hz, 2H), 3.80-3.68 (m, 1H), 3.50-3.38 (m, 4H), 3.10-3.04 (m, 4H), 2.69-2.58 (m, 1H), 2.48 (s, 1H), 2.19-2.07 (m, 1H), 2.00 (m, J=4.8, 13.3 Hz, 1H), 1.42 (s, 9H).

### Step 3-3-(4-Piperazin-1-ylphenyl)piperidine-2,6-dione

To a solution of tert-butyl 4-[4-(2,6-dioxo-3-piperidyl)phenyl]piperazine-1-carboxylate (240 mg, 642.66 μmol) in DCM (3 mL) was added TFA (1 mL), then the mixture was stirred at 25° C. for 1 hr. On completion, the reaction was concentrated in vacuo to give the title compound (248 mg, 99% yield, TFA) as brown solid. LC-MS (ESI⁺) m/z 274.1 (M+H)⁺.

### Synthesis of Tert-butyl N-(4-formyl-1-bicyclo[2.2.2]octanyl)carbamate (Intermediate RR)

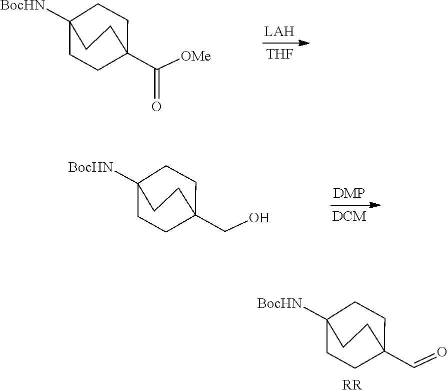

### Step 1—Tert-butyl N-[4-(hydroxymethyl)-1-bicyclo[2.2.2]octanyl]carbamate

To a solution of methyl 4-(tert-butoxycarbonylamino)bicyclo[2.2.2]octane-1-carboxylate (0.5 g, 1.76 mmol, CAS #943845-74-7) in THF (10 mL) was added LAH (2.5 M, 1.41 mL) slowly at −70° C., the mixture was stirred at −70° C. for 3 hrs under N₂. On completion, the mixture was quenched with water (0.2 ml) and 15% NaOH (0.2 mL) and more water (0.6 mL) at 0° C. The reaction mixture was then diluted with additional water (5 mL) and extracted with EA (20 mL×2). The combined organic layers were washed with water (10 mL×2), dried over Na₂SO₄, filtered and concentrated under reduced pressure to give the title compound (360 mg, 79% yield) as white solid. ¹H NMR (400 MHz, DMSO-d₆) δ 6.27 (s, 1H), 4.32-4.26 (m, 1H), 2.99 (d, J=5.6 Hz, 2H), 1.78-1.59 (m, 7H), 1.37-1.33 (m, 14H).

### Step 2—Tert-butyl N-(4-formyl-1-bicyclo[2.2.2]octanyl)carbamate

To a solution of tert-butyl N-[4-(hydroxymethyl)-1-bicyclo[2.2.2]octanyl]carbamate (360 mg, 1.41 mmol) in DCM (5 mL) was added DMP (657 mg, 1.55 mmol), the mixture was then stirred at 25° C. for 1 hr. On completion, the reaction mixture was quenched with Na₂S₂O₃ (3 mL) and NaHCO₃ (3 mL). Then, the mixture was extracted with DCM (10 mL×2). The combined organic layers were washed with water (8 mL×2), dried over Na₂SO₄, filtered and concentrated in vacuo. The crude product was triturated with PE at 25° C. for 10 mins to give the title compound (300 mg, 84% yield) as white solid. ¹H NMR (400 MHz, DMSO-d₆) δ 9.39 (s, 1H), 6.55-6.33 (m, 1H), 1.79-1.69 (m, 6H), 1.64-1.55 (m, 5H), 1.36 (s, 9H), 0.89-0.77 (m, 1H).

### Synthesis of 3-[4-[4-[(4-amino-1-bicyclo[2.2.2]octanyl)methyl]piperazin-1-yl]phenyl]piperidine-2,6-dione (Intermediate RS)

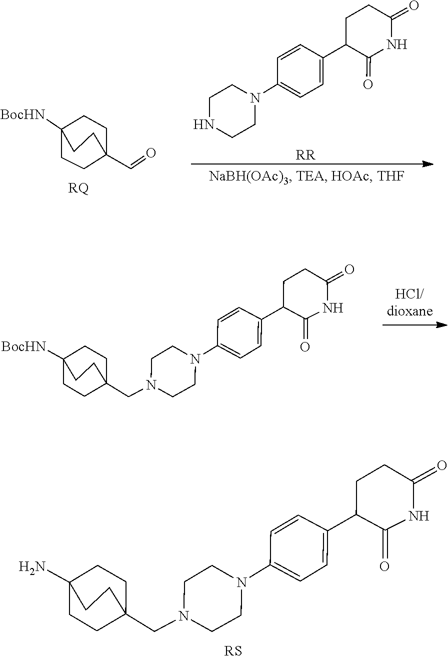

### Step 1—Tert-butyl N-[4-[[4-[4-(2,6-dioxo-3-piperidyl)phenyl]piperazin-1-yl]methyl]-1-bicyclo[2.2.2]octanyl]carbamate

To a mixture of 3-(4-piperazin-1-ylphenyl)piperidine-2,6-dione (248 mg, 640 μmol, TFA, Intermediate RQ) in THF (5 mL) was added TEA (194 mg, 1.92 mmol) until pH=8. The mixture was stirred at 25° C. for 10 min, then HOAc (76.8 mg, 1.28 mmol) was added until the pH=6 at 25° C. Subsequently, tert-butyl N-(4-formyl-1-bicyclo[2.2.2]octanyl)carbamate (162 mg, 640 μmol, Intermediate RR) was added and the mixture was stirred at 25° C. for 20 min. Next, NaBH(OAc)₃ (271 mg, 1.28 mmol) was added one portion and the resulting reaction mixture was stirred at 25° C. for 1 hr. On completion, the reaction mixture was quenched by water (0.5 mL) and diluted with EA (20 mL), then filtered and the filter cake was collected. The residue was purified by prep-HPLC (The residue was purified by prep-HPLC (column: Phenomenex luna C18 150*25 mm*10 um; mobile phase: [water (FA)-ACN]; gradient:88%-388% B over 10 min) to give the title compound (100 mg, 30% yield) as white solid. ¹HNMR (400 MHz, DMSO-d₆) δ 10.78 (s, 1H), 8.98-8.69 (m, 1H), 7.21-6.77 (m, 3H), 6.53-6.20 (m, 1H), 3.79-3.45 (m, 4H), 3.23-2.96 (m, 5H), 2.69-2.54 (m, 3H), 2.21-1.94 (m, 3H), 1.83-1.45 (m, 12H), 1.36 (s, 9H).

### Step 2—3-[4-[4-[(4-Amino-1-bicyclo[2.2.2]octanyl)methyl]piperazin-1-yl]phenyl]piperidine-2,6-dione

To a solution of tert-butyl N-[4-[[4-[4-(2,6-dioxo-3-piperidyl)phenyl]piperazin-1-yl]methyl]-1-bicyclo[2.2.2]octanyl]carbamate (70 mg, 137 μmol) in dioxane (1 mL) was added HCl/dioxane (0.5 mL), then the mixture was stirred at 25° C. for 1 hr. On completion, the reaction was concentrated under reduced pressure to give the title compound (60 mg, 97% yield, HCl) as a yellow solid. LC-MS (ESI⁺) m/z 411.1 (M+H)⁺.

### Synthesis of Tert-butyl N-[4-[[4-[4-(2,6-dibenzyloxy-3-pyridyl)-2-fluoro-phenyl]piperazin-1-yl]methyl]-1-methyl-cyclohexyl]carbamate (Intermediate RT)

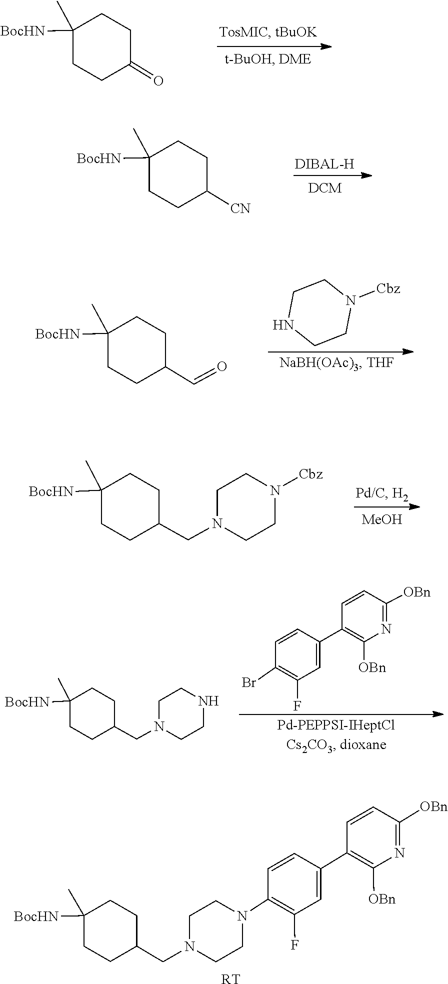

### Step 1—Tert-butyl N-(4-cyano-1-methyl-cyclohexyl)carbamate

To a solution of tert-butyl N-(1-methyl-4-oxo-cyclohexyl)carbamate (4 g, 17.6 mmol, CAS#412293-43-7) and 1-(isocyanomethylsulfonyl)-4-methyl-benzene (3.78 g, 19.3 mmol) in DME (100 mL) was added the solution oft-BuOK (3.95 g, 35.2 mmol) in t-BuOH (6.52 g, 87.9 mmol, 8.42 mL) and DME (10 mL) at 0° C. The reaction was stirred at 25° C. for 2 hrs. On completion the reaction was diluted with MTBE (40 mL). The organic layer was washed with water (40 mL), dried over Na₂SO₄ and concentrated in vacuo. The residue was purified by column chromatography (SiO₂, Petroleum ether/Ethyl acetate=10/1 to 2/1) to give the title compound (7 g, 83% yield) as yellow solid. ¹H NMR (400 MHz, CDCl₃) δ 4.22 (s, 1H), 2.75-2.35 (m, 1H), 2.06 (d, J=14.0 Hz, 1H), 1.94 (s, 1H), 1.88-1.54 (m, 6H), 1.37 (d, J=6.0 Hz, 9H), 1.26 (d, J=8.4 Hz, 3H).

### Step 2—Tert-butyl N-(4-formyl-1-methyl-cyclohexyl)carbamate

To a solution of tert-butyl N-(4-cyano-1-methyl-cyclohexyl)carbamate (6 g, 25.1 mmol) in DCM (70 mL) was added DIBAL-H (1 M, 50.3 mL) at −78° C. The reaction was stirred at −78° C. for 2 hrs. On completion, the reaction was quenched with HCl (2N, 10 mL). The mixture was extracted with DCM (200 mL×2). The organic layer was washed with water (100 ml), dried over Na₂SO₄ and concentrated in vacuo to give the title compound (6 g, 98% yield) as yellow oil. ¹H NMR (400 MHz, CDCl₃) δ 9.63-9.53 (m, 1H), 2.28-2.13 (m, 1H), 1.85-1.70 (m, 4H), 1.68-1.45 (m, 4H), 1.36 (d, J=4.0 Hz, 9H), 1.27-1.20 (m, 3H).

### Step 3—Benzyl 4-[[4-(tert-butoxycarbonylamino)-4-methyl-cyclohexyl]methyl]piperazine-1-carboxylate

A solution of tert-butyl N-(4-formyl-1-methyl-cyclohexyl)carbamate (6 g, 24.8 mmol), benzyl piperazine-1-carboxylate (4.38 g, 19.8 mmol, 3.84 mL, CAS #31166-44-6) and HOAc (1.49 g, 24.8 mmol, 1.42 mL) in THF (100 mL) was stirred at 25° C. for 0.5 hr. Then, NaBH(OAc)₃ (6.32 g, 29.8 mmol) was added and the mixture was stirred at 25° C. for 1 hr. On completion, the reaction was quenched with water (2 mL) and concentrated in vacuo. The residue was purified by prep-HPLC (column: Welch Ultimate XB-SiOH 250*50*10 um; mobile phase: [Hexane-EtOH]; gradient: 2%-19% B over 25 min) to give the title compound (11 g, 99% yield) as yellow oil. ¹H NMR (400 MHz, CDCl₃) δ 7.32-7.22 (m, 5H), 5.06 (s, 2H), 3.45 (s, 4H), 2.33 (s, 4H), 2.18-2.10 (m, 2H), 2.00 (s, 3H), 1.73 (d, J=12.4 Hz, 1H), 1.66-1.52 (m, 3H), 1.36 (s, 9H), 1.25-1.21 (m, 3H), 1.07-0.96 (m, 2H).

### Step 4—Tert-butyl N-[1-methyl-4-(piperazin-1-ylmethyl)cyclohexyl]carbamate

To a solution of benzyl 4-[[4-(tert-butoxycarbonylamino)-4-methyl-cyclohexyl]methyl]piperazine-1-carboxylate (3.5 g, 7.85 mmol,) in MeOH (40 mL) was added Pd/C (3.50 g, 3.29 mmol, 10 wt %) under N₂. The suspension was degassed under vacuum and purged with H₂ several times. The mixture was stirred under H₂ (15 psi) at 25° C. for 4 hrs. On completion, the reaction was filtered and the filtrate was concentrated in vacuo to give the title compound (2.4 g, 98% yield) as white solid. ¹H NMR (400 MHz, CDCl₃) δ 4.52-4.24 (m, 1H), 2.95 (t, J=4.8 Hz, 3H), 2.44 (s, 4H), 2.21-2.15 (m, 2H), 2.01 (d, J=10.8 Hz, 2H), 1.82 (dd, J=4.0, 11.6 Hz, 1H), 1.73-1.59 (m, 3H), 1.57-1.49 (m, 1H), 1.46-1.43 (m, 9H), 1.36-1.30 (m, 3H), 1.22 (dt, J=3.2, 13.6 Hz, 1H), 1.16-1.01 (m, 2H).

### Step 5—Tert-butyl N-[4-[[4-[4-(2,6-dibenzyloxy-3-pyridyl)-2-fluoro-phenyl]piperazin-1-yl]methyl]-1-methyl-cyclohexyl]carbamate

A mixture of 2,6-dibenzyloxy-3-(4-bromo-3-fluoro-phenyl)pyridine (2 g, 4.31 mmol, synthesized via Step 1 of Intermediate PM), tert-butyl N-[1-methyl-4-(piperazin-1-ylmethyl)cyclohexyl]carbamate (1.61 g, 5.17 mmol), Cs₂CO₃ (4.21 g, 12.9 mmol), and 1,3-bis[2,6-bis(1-propylbutyl)phenyl]-4,5-dichloro-2H-imidazol-1-ium-2-ide 3-chloropyridine dichloropalladium (419 mg, 430 μmol) in dioxane (50 mL) was stirred at 110° C. for 16 hrs under N₂. On completion, the reaction was diluted with EA (200 mL). The organic layer was washed with water (150 mL), dried over Na₂SO₄ and concentrated in vacuo. The residue was purified by column chromatography (SiO₂, DCM/Ethyl acetate=I/O to 5/1) to give the title compound (1.7 g, 56% yield) as brown solid. ¹H NMR (400 MHz, DMSO-d₆) δ 7.76 (d, J=8.0 Hz, 1H), 7.45-7.31 (m, 12H), 7.02 (t, J=9.2 Hz, 1H), 6.54 (d, J=8.0 Hz, 1H), 6.42-6.09 (m, 1H), 5.39 (d, J=19.6 Hz, 4H), 3.02 (s, 4H), 2.48-2.47 (m, 2H), 2.15 (dd, J=6.8, 14.8 Hz, 3H), 1.74-1.45 (m, 6H), 1.38 (d, J=2.4 Hz, 9H), 1.22-1.18 (m, 3H), 1.15-0.97 (m, 4H).

### Synthesis of Tert-butyl N-[4-[[4-[4-(2,6-dibenzyloxy-3-pyridyl)-2-fluoro-phenyl]piperazin-1-yl]methyl]-1-methyl-cyclohexyl]carbamate (Intermediate RU) and tert-butyl N-[4-[[4-[4-(2,6-dibenzyloxy-3-pyridyl)-2-fluoro-phenyl]piperazin-1-yl]methyl]-1-methyl-cyclohexyl]carbamate (Intermediate RV)

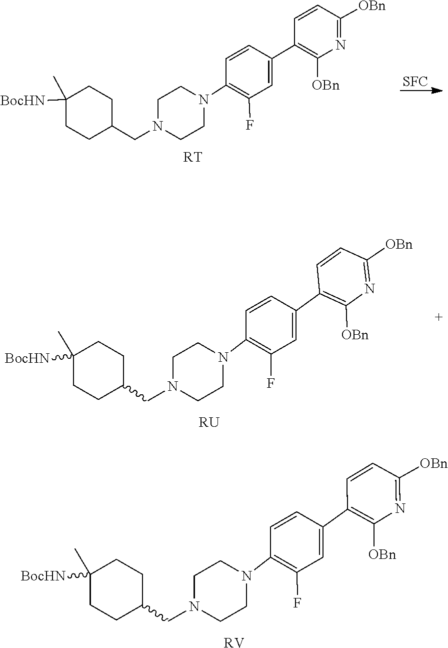

Tert-butyl N-[4-[[4-[4-(2,6-dibenzyloxy-3-pyridyl)-2-fluoro-phenyl]piperazin-1-yl]methyl]-1-methyl-cyclohexyl]carbamate (Intermediate RT) was separated into diastereomers by SFC (column: DAICEL CHIRALPAK AD (250 mm*30 mm, 10 um); mobile phase: [CO₂-ACN/MeOH (0.1% NH₃H₂O)]; B %:45%, isocratic elution mode). The first fraction tert-butyl N-[4-[[4-[4-(2,6-dibenzyloxy-3-pyridyl)-2-fluoro-phenyl]piperazin-1-yl]methyl]-1-methyl-cyclohexyl]carbamate (750 mg, 44% yield, peak 1) was isolated as a pink solid (¹H NMR (400 MHz, DMSO-d₆) δ 7.75 (d, J=8.0 Hz, 1H), 7.50-7.25 (m, 12H), 7.06-6.97 (m, 1H), 6.53 (d, J=8.0 Hz, 1H), 6.24-6.07 (m, 1H), 5.39 (d, J=19.6 Hz, 4H), 3.10-2.94 (m, 4H), 2.48-2.44 (m, 4H), 2.12 (d, J=6.8 Hz, 4H), 1.54-1.43 (m, 3H), 1.37 (s, 9H), 1.17 (s, 3H), 1.14-1.00 (m, 4H). The second fraction tert-butyl N-[4-[[4-[4-(2,6-dibenzyloxy-3-pyridyl)-2-fluoro-phenyl]piperazin-1-yl]methyl]-1-methyl-cyclohexyl]carbamate (600 mg, 35% yield, peak 2) was isolated as yellow solid (¹H NMR (400 MHz, DMSO-d₆) δ 7.75 (d, J=8.0 Hz, 1H), 7.46-7.26 (m, 12H), 7.02 (t, J=8.8 Hz, 1H), 6.53 (d, J=8.0 Hz, 1H), 6.34 (s, 1H), 5.39 (d, J=19.6 Hz, 4H), 3.02 (s, 4H), 2.47-2.40 (m, 4H), 2.16 (d, J=7.2 Hz, 2H), 1.73-1.48 (m, 7H), 1.37 (s, 9H), 1.20 (s, 3H), 1.08-0.97 (m, 2H)). The absolute stereochemistry of the diastereomers was unknown.

### Synthesis of 3-[4-[4-[(4-Amino-4-methyl-cyclohexyl)methyl]piperazin-1-yl]-3-fluoro-phenyl]piperidine-2,6-dione (Intermediate RW)

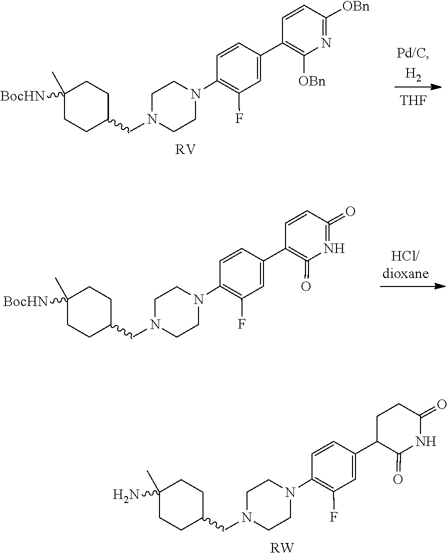

### Step 1—Tert-butyl N-[4-[[4-[4-(2,6-dioxo-3-piperidyl)-2-fluoro-phenyl]piperazin-1-yl] methyl]-1-methyl-cyclohexyl]carbamate

To a solution of tert-butyl N-[4-[[4-[4-(2,6-dibenzyloxy-3-pyridyl)-2-fluoro-phenyl]piperazin-1-yl]methyl]-1-methyl-cyclohexyl]carbamate (300 mg, 431 μmol, Intermediate RV) in THF (5 mL) was added Pd/C (300 mg, 281 mol, 10 wt %) under N₂. The suspension was degassed under vacuum and purged with H₂ several times. The mixture was stirred under H₂ (15 psi) at 25° C. for 16 hrs. On completion, the reaction was filtered and the filtrate was concentrated in vacuo to give the title compound (200 mg, 89% yield) as yellow solid. ¹H NMR (400 MHz, DMSO-d₆) δ 10.81 (s, 1H), 7.05-6.90 (m, 3H), 3.79 (dd, J=4.8, 11.6 Hz, 1H), 3.29 (s, 1H), 2.99 (s, 4H), 2.71-2.58 (m, 2H), 2.47-2.44 (m, 3H), 2.25-2.14 (m, 3H), 2.04-1.98 (m, 1H), 1.75-1.42 (m, 8H), 1.37 (s, 9H), 1.20 (s, 3H), 1.10-0.95 (m, 2H).

### Step 2—3-[4-[4-[(4-Amino-4-methyl-cyclohexyl)methyl]piperazin-1-yl]-3-fluoro-phenyl]piperidine-2,6-dione

A mixture of tert-butyl N-[4-[[4-[4-(2,6-dioxo-3-piperidyl)-2-fluoro-phenyl]piperazin-1-yl]methyl]-1-methyl-cyclohexyl]carbamate (100 mg, 193 μmol) in HCl/dioxane (4 M, 3 mL) and DCM (0.5 mL) was stirred at 40° C. for 0.5 hr. On completion, the reaction was concentrated in vacuo to give the title compound (87 mg, 99% yield, HCl) as white solid. LC-MS (ESI⁺) m/z 417.1 (M+H)⁺.

### Synthesis of 3-[4-[4-[[(3R,4R)-4-amino-3-fluoro-1-piperidyl]methyl]-1-piperidyl]-3-fluoro-phenyl]piperidine-2,6-dione (Intermediate RY)

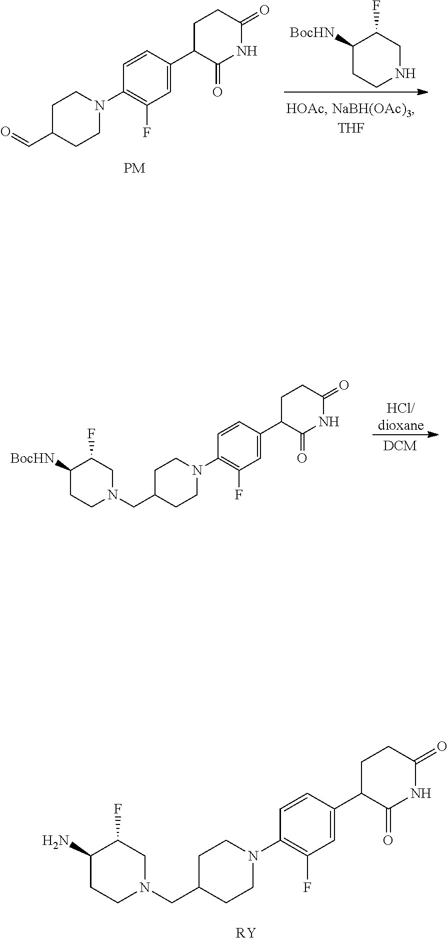

### Step 1—Tert-butyl N-[(3R,4R)-1-[[1-[4-(2,6-dioxo-3-piperidyl)-2-fluoro-phenyl]-4-piperidyl]methyl]-3-fluoro-4-piperidyl]carbamate

To a solution of tert-butyl N-[(3R,4R)-3-fluoro-4-piperidyl]carbamate (137 mg, 628 μmol, CAS#1268520-95-1) and 1-[4-(2,6-dioxo-3-piperidyl)-2-fluoro-phenyl] piperidine-4-carbaldehyde (200 mg, 628 mol, Intermediate PM) in THF (2 mL) was added HOAc (36.0 μL, 628 mol), and the mixture was stirred at 25° C. for 0.5 hr. Then, NaBH(OAc)₃ (266 mg, 1.26 mmol) was added and the mixture was stirred at 25° C. for 1 hr. On completion, the mixture was quenched with water (0.5 mL) and filtered to give the filtrate. The filtrate was purified by prep-HPLC (column: Phenomenex luna C18 150*25 mm*10 um; mobile phase: [water(FA)-ACN]; gradient:6%-36% B over 10 min) to give the title compound (200 mg, 61% yield) as a white solid. ¹H NMR (400 MHz, DMSO-d₆) δ 10.80 (s, 1H), 7.04-6.90 (m, 4H), 4.42-4.19 (m, 1H), 3.81-3.77 (m, 1H), 3.14-3.06 (m, 1H), 2.79-2.57 (m, 6H), 2.48-2.44 (m, 1H), 2.37-2.05 (m, 4H), 2.03-1.88 (m, 3H), 1.76-1.73 (m, 3H), 1.65-1.56 (m, 1H), 1.45-1.41 (m, 1H), 1.38 (s, 9H), 1.30-1.17 (m, 2H) LC-MS (ESI⁺) m/z 521.0 (M+H)⁺.

### Step 2—3-[4-[4-[[(3R,4R)-4-amino-3-fluoro-1-piperidyl]methyl]-1-piperidyl]-3-fluoro-phenyl]piperidine-2,6-dione

To a solution of tert-butyl N-[(3R,4R)-1-[[1-[4-(2,6-dioxo-3-piperidyl)-2-fluoro-phenyl]-4-piperidyl] methyl]-3-fluoro-4-piperidyl]carbamate (115 mg, 221 μmol) in DCM (2 mL) was added HCl/dioxane (2 mL), then the mixture was stirred at 40° C. for 1 hr. On completion, the mixture concentrated in vacuo to give the title compound (96.0 mg, 95% yield, HCl) as a white solid. LC-MS (ESI⁺) m/z 421.1 (M+H)⁺.

### Synthesis of 3-[4-[4-[(4-Amino-2-oxabicyclo[2.2.2]octan-1-yl)methyl]piperazin-1-yl]-3-fluoro-phenyl] piperidine-2,6-dione (Intermediate RZ)

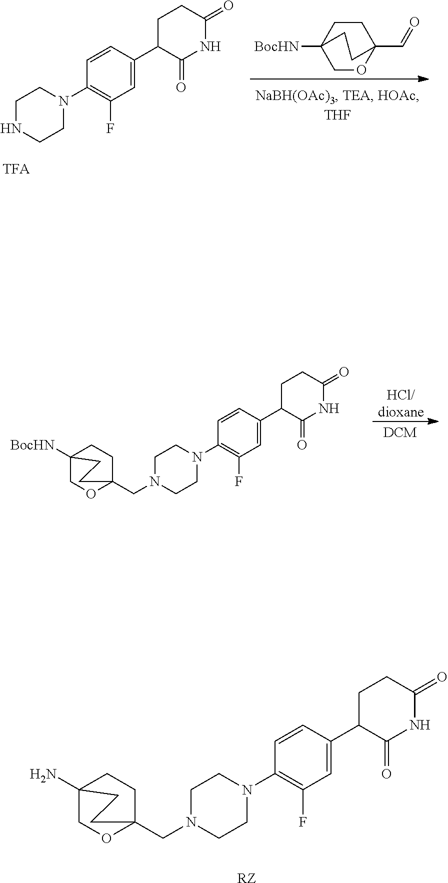 [SMILES: O=C1CCC(c2ccc(N3CCNCC3)c(F)c2)C(=O)N1 ; CC(C)(C)OC(=O)NC12CCC(C=O)(CC1)OC2 ; CC(C)(C)OC(=O)NC12CCC(CN3CCN(c4ccc(C5CCC(=O)NC5=O)cc4F)CC3)(CC1)OC2 ; NC12CCC(CN3CCN(c4ccc(C5CCC(=O)NC5=O)cc4F)CC3)(CC1)OC2 ; "RZ"]

### Step 1—Tert-butyl N-[1-[[4-[4-(2,6-dioxo-3-piperidyl)-2-fluoro-phenyl]piperazin-1-yl]methyl]-2-oxabicyclo[2.2.2]octan-4-yl]carbamate

To a solution of 3-(3-fluoro-4-piperazin-1-yl-phenyl)piperidine-2,6-dione (200 mg, 493 μmol, TFA, Intermediate OP) in THF (2 mL) was added TEA (206 μL, 1.48 mmol) and HOAc (56.5 μL, 987 mol) to adjust pH=6-7. Then tert-butyl N-(1-formyl-2-oxabicyclo[2.2.2]octan-4-yl)carbamate (126 mg, 493 μmol, CAS #1417551-42-8) was added and the mixture was stirred at 25° C. for 0.5 hr. Next, NaBH(OAc)₃ (157 mg, 740 μmol) was added and the mixture was stirred at 25° C. for 1.5 hrs. On completion, the mixture was quenched with water (1 mL) and concentrated in vacuo to give the residue. The residue was purified by prep-HPLC (column: Phenomenex luna C18 150*25 mm*10 um; mobile phase: [water (FA)-ACN]; gradient: 10%-40% B over 10 min) to give the title compound (130 mg, 49% yield) as purple solid. ¹H NMR (400 MHz, DMSO-d₆) δ 10.82 (s, 1H), 7.10-6.91 (m, 3H), 6.71-6.46 (m, 1H), 3.88-3.70 (m, 3H), 3.30-2.87 (m, 6H), 2.74-2.52 (m, 4H), 2.47 (s, 1H), 2.30-2.10 (m, 2H), 2.06-1.85 (m, 5H), 1.81-1.78 (m, 2H), 1.63 (s, 2H), 1.36 (s, 9H). LC-MS (ESI⁺) m/z 531.2 (M+H)⁺.

### Step 2—3-[4-[4-[(4-Amino-2-oxabicyclo[2.2.2]octan-1-yl)methyl]piperazin-1-yl]-3-fluoro-phenyl]piperidine-2,6-dione

A solution of tert-butyl N-[1-[[4-[4-(2,6-dioxo-3-piperidyl)-2-fluoro-phenyl]piperazin-1-yl]methyl]-2-oxabicyclo[2.2.2]octan-4-yl]carbamate (100 mg, 188 μmol) in DCM (1 mL) and dioxane (2 mL) was stirred at 40° C. for 1.5 hr. On completion, the mixture was concentrated in vacuo to give the title compound (85.0 mg, 96% yield, HCl) as yellow solid. LC-MS (ESI⁺) m/z 431.1 (M+H)⁺.

### Synthesis of 3-[4-[4-[(4-Aminocyclohexyl)methyl]piperazin-1-yl]-2-fluoro-5-methyl-phenyl]piperidine-2,6-dione (Intermediate SA)

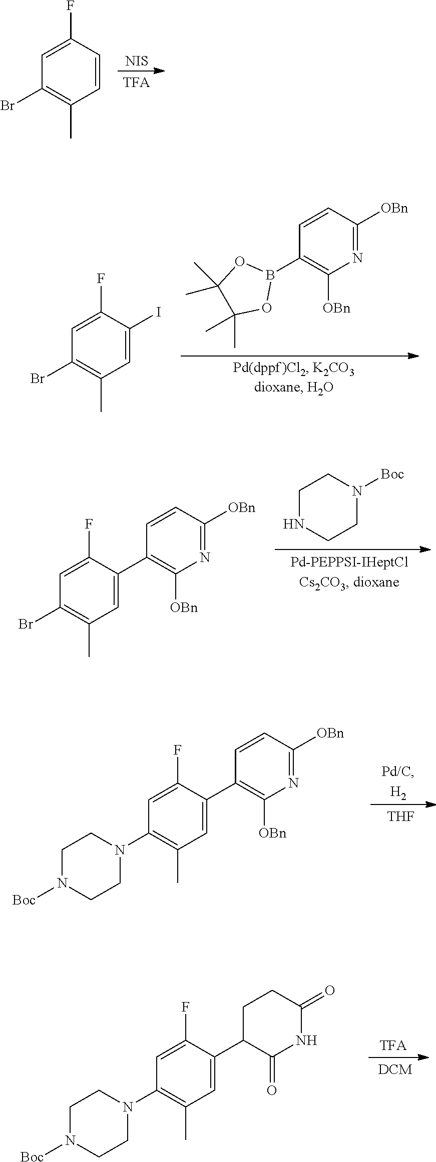

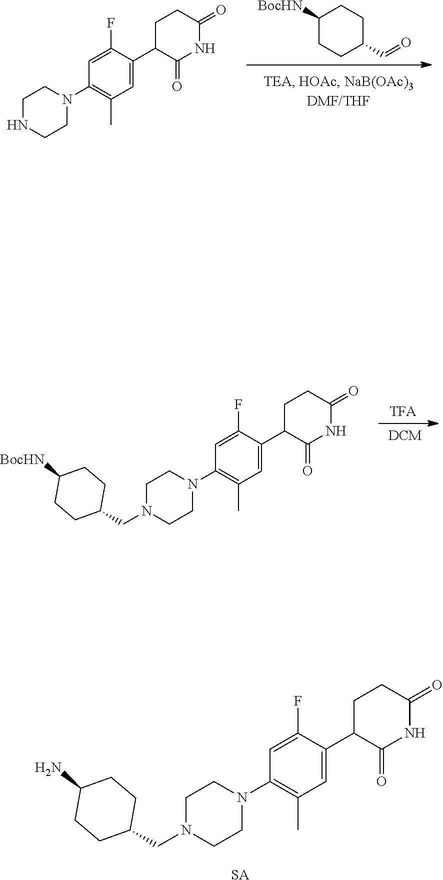

### Step 1—1-Bromo-5-fluoro-4-iodo-2-methyl-benzene

To a solution of 2-bromo-4-fluoro-1-methyl-benzene (3.8 g, 20.1 mmol, CAS #1422-53-3) in TFA (25 mL) was added NIS (4.98 g, 22.1 mmol). The mixture was then stirred at 20° C. for 19 hrs. On completion, the reaction mixture was concentrated in vacuo. The crude product was triturated with solution (PE (15 mL), EA (10 mL)) and filtered. The filtrate was diluted with EA (40 mL), washed with saturated Na₂S₂O₃ solution (30 mL×2) and water (30 mL×5), dried over Na₂SO₄, filtered and concentrated in vacuo to give the title compound (6.33 g, 100% yield) as yellow oil. ¹H NMR (400 MHz, DMSO-d₆) δ 7.86 (d, J=6.8 Hz, 1H), 7.58 (d, J=7.6 Hz, 1H), 2.29 (s, 3H).

### Step 2—2,6-Dibenzyloxy-3-(4-bromo-2-fluoro-5-methyl-phenyl)pyridine

A mixture of 1-bromo-5-fluoro-4-iodo-2-methyl-benzene (1 g, 3.18 mmol), 2,6-dibenzyloxy-3-(4,4,5,5-tetramethyl-1,3,2-dioxaborolan-2-yl)pyridine (1.06 g, 2.54 mmol, CAS #2152673-80-6), K₂CO₃ (1.32 g, 9.53 mmol) and Pd(dppf)Cl₂·CH₂Cl₂ (259 mg, 317 μmol) in dioxane (18 mL) and H₂O (3.6 mL) was stirred at 80° C. for 2 hrs under N₂. On completion, the reaction mixture was diluted with EA (50 mL) and filtered. The organic layer was concentrated in vacuo. The residue was purified by column chromatography (SiO₂, EA in PE, 5%) to give the title compound (735 mg, 48% yield) as colorless oil. ¹H NMR (400 MHz, DMSO-d₆) δ 7.66 (d, J=8.0 Hz, 1H), 7.58 (d, J=9.6 Hz, 1H), 7.45-7.31 (m, 10H), 7.30-28 (m, 1H), 6.56 (d, J=8.0 Hz, 1H), 5.37 (d, J=3.6 Hz, 4H), 2.32 (s, 3H). LC-MS (ESI⁺) m/z 479.8 (M+H)⁺.

### Step 3—Tert-butyl 4-[4-(2,6-dibenzyloxy-3-pyridyl)-5-fluoro-2-methyl-phenyl]piperazine-1-carboxylate

A mixture of 2,6-dibenzyloxy-3-(4-bromo-2-fluoro-5-methyl-phenyl)pyridine (660 mg, 1.38 mmol), tert-butyl piperazine-1-carboxylate (385 mg, 2.07 mmol, CAS #57260-71-6), Cs₂CO₃ (1.35 g, 4.14 mmol) and Pd-PEPPSI-IHeptCl (134 mg, 137 μmol) in dioxane (10 mL) was stirred at 100° C. under N₂ for 16 hrs. On completion, the reaction was diluted with EA (80 mL) and washed with water (50 mL×3). The organic layer was dried over Na₂SO₄, filtered and concentrated in vacuo. The residue was purified by column chromatography (SiO₂, PE:EA=20:1 to 20:1.5) to give the title compound (538 mg, 66% yield) as brown solid. ¹H NMR (400 MHz, DMSO-d₆) δ 7.60 (d, J=8.0 Hz, 1H), 7.48-7.23 (m, 10H), 7.18 (d, J=8.8 Hz, 1H), 6.88 (d, J=12.0 Hz, 1H), 6.52 (d, J=8.0 Hz, 1H), 5.35 (d, J=4.0 Hz, 4H), 3.55-3.40 (m, 4H), 2.88-2.75 (m, 4H), 2.22 (s, 3H), 1.42 (s, 9H); LC-MS (ESI⁺) m/z 584.1 (M+H)⁺.

### Step 4—Tert-butyl 4-[4-(2,6-dioxo-3-piperidyl)-5-fluoro-2-methyl-phenyl]piperazine-1-carboxylate

To a solution of tert-butyl 4-[4-(2,6-dibenzyloxy-3-pyridyl)-5-fluoro-2-methyl-phenyl]piperazine-1-carboxylate (538 mg, 921 μmol) in THF (20 mL) was added Pd/C (300 mg, 10 wt %) under Ar. The suspension was degassed under vacuum and purged with H₂ several times. The mixture was stirred under H₂ (15 psi) at 25° C. for 4 hrs. On completion, the reaction was diluted with THF (200 mL) and filtered. The filtrate was concentrated in vacuo to give the title compound (361 mg, 96% yield) as brown solid. ¹H NMR (400 MHz, DMSO-d₆) δ 10.83 (s, 1H), 7.07 (d, J=8.8 Hz, 1H), 6.83 (d, J=12.4 Hz, 1H), 3.92 (dd, J=5.2, 12.4 Hz, 1H), 3.52-3.39 (m, 4H), 2.82-2.75 (m, 4H), 2.75-2.65 (m, 1H), 2.55-2.52 (m, 1H), 2.20 (s, 3H), 2.18-2.10 (m, 1H), 1.99-1.92 (m, 1H), 1.42 (s, 9H); LC-MS (ESI⁺) m/z 406.0 (M+H)⁺.

### Step 5—3-(2-Fluoro-5-methyl-4-piperazin-1-yl-phenyl)piperidine-2,6-dione

A mixture of tert-butyl 4-[4-(2,6-dioxo-3-piperidyl)-5-fluoro-2-methyl-phenyl]piperazine-1-carboxylate (200 mg, 493 μmol) in DCM (2.1 mL) and TFA (1.07 g, 9.42 mmol, 0.7 mL) was stirred at 25° C. for 0.5 hr. On completion, the reaction mixture was concentrated in vacuo to give the title compound (206 mg, 99% yield, TFA) as brown oil. LC-MS (ESI⁺) m/z 306.0 (M+H)⁺.

### Step 6—Tert-butyl N-[4-[[4-[4-(2,6-dioxo-3-piperidyl)-5-fluoro-2-methyl-phenyl]piperazin-1-yl]methyl]cyclohexyl]carbamate

To a solution of 3-(2-fluoro-5-methyl-4-piperazin-1-yl-phenyl)piperidine-2,6-dione (206 mg, 491 mol, TFA) in THF (3 mL) and DMF (1 mL) was added TEA (99.4 mg, 982 mol), HOAc (88.5 mg, 1.47 mmol) and tert-butyl N-(4-formylcyclohexyl)carbamate (111 mg, 491 mol, CAS #181308-57-6) at −10° C. After stirring 0.25 hr, NaBH(OAc)₃ (156 mg, 736 mol) was added and the mixture was stirred at −10° C. for another 0.25 hr. On completion, the reaction mixture was quenched with water (0.2 mL) and filtered. The filter cake was washed with ACN (10 mL) and water (5 mL) and concentrated in vacuo to give the title compound (115 mg, 45% yield) as brown solid. ¹H NMR (400 MHz, DMSO-d₆) δ 10.83 (s, 1H), 7.05 (d, J=8.8 Hz, 1H), 6.80 (d, J=12.4 Hz, 1H), 6.70 (d, J=8.0 Hz, 1H), 3.91 (dd, J=4.8, 12.4 Hz, 1H), 3.22-3.08 (m, 1H), 2.82 (s, 4H), 2.77-2.68 (m, 1H), 2.49-2.39 (m, 4H), 2.17 (s, 4H), 2.13 (d, J=7.2 Hz, 2H), 2.00-1.91 (m, 1H), 1.77 (d, J=11.2 Hz, 4H), 1.37 (s, 9H), 1.19-1.04 (m, 3H), 0.95-0.78 (m, 3H); LC-MS (ESI⁺) m/z 517.3 (M+H)⁺.

### Step 7—3-[4-[4-[(4-aminocyclohexyl)methyl]piperazin-1-yl]-2-fluoro-5-methyl-phenyl]piperidine-2,6-dione

A mixture of tert-butyl N-[4-[[4-[4-(2,6-dioxo-3-piperidyl)-5-fluoro-2-methyl-phenyl]piperazin-1-yl] methyl]cyclohexyl]carbamate (55 mg, 106 mol) in DCM (0.9 mL) and TFA (460 mg, 4.04 mmol, 0.3 mL) was stirred at 25° C. for 0.5 hr. On completion, the reaction mixture was concentrated in vacuo to give the title compound (56.4 mg, 99% yield, TFA) as brown oil. LC-MS (ESI⁺) m/z 417.2 (M+H)⁺.

### Synthesis of 3-(3-Chloro-4-piperazin-1-yl-phenyl)piperidine-2,6-dione (Intermediate SB)

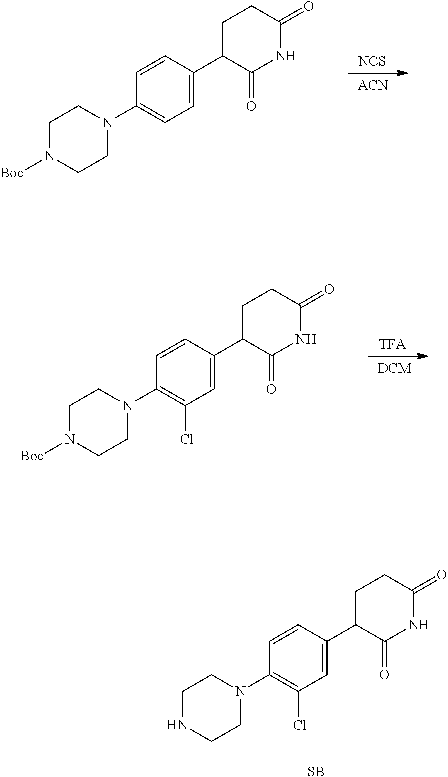

### Step 1—Tert-butyl 4-[2-chloro-4-(2,6-dioxo-3-piperidyl)phenyl]piperazine-1-carboxylate

To the solution of tert-butyl 4-[4-(2,6-dioxo-3-piperidyl)phenyl]piperazine-1-carboxylate (800 mg, 2.14 mmol, synthesized via Steps 1-2 of Intermediate RQ) in ACN (16 mL) was added NCS (257 mg, 1.93 mmol), and the mixture was stirred at 90° C. for 1 hr. On completion, the mixture was quenched with water (0.5 mL), diluted with water (30 mL), and extracted with EA (3×20 mL). The combined organic layer was dried over anhydrous Na₂SO₄, concentrated in vacuo to give the residue. The residue was purified by silica gel column chromatography (SiO₂, PE:EA=1:0 to 1:0) to give the title compound (650 mg, 74% yield) as a purple oil. ¹HNMR (400 MHz, DMSO-d₆) δ 10.83 (s, 1H), 7.31 (d, J=1.6 Hz, 1H), 7.18-7.07 (m, 2H), 3.83 (dd, J=4.8, 12.0 Hz, 1H), 3.48-3.45 (m, 4H), 2.94-2.86 (m, 4H), 2.70-2.61 (m, 1H), 2.52-2.51 (m, 1H), 2.23-2.18 (m, 1H), 2.04-1.99 (m, 1H), 1.42 (s, 9H). LC-MS (ESI⁺) m/z 408.0 (M+H)⁺.

### Step 3—3-(3-Chloro-4-piperazin-1-yl-phenyl)piperidine-2,6-dione

To a solution of tert-butyl 4-[2-chloro-4-(2,6-dioxo-3-piperidyl)phenyl]piperazine-1-carboxylate (570 mg, 1.40 mmol) in DCM (5 mL) was added TFA (2.8 mL), then the mixture was stirred at 25° C. for 0.5 hr. On completion, the mixture was concentrated in vacuo to give the title compound (580 mg, 98% yield, TFA) as a light yellow oil. LC-MS (ESI⁺) m/z 307.9 (M+H)⁺.

### Synthesis of 3-[4-[4-[(4-Aminocyclohexyl)methyl]piperazin-1-yl]-3-chloro-phenyl]piperidine-2,6-dione (Intermediate SC)

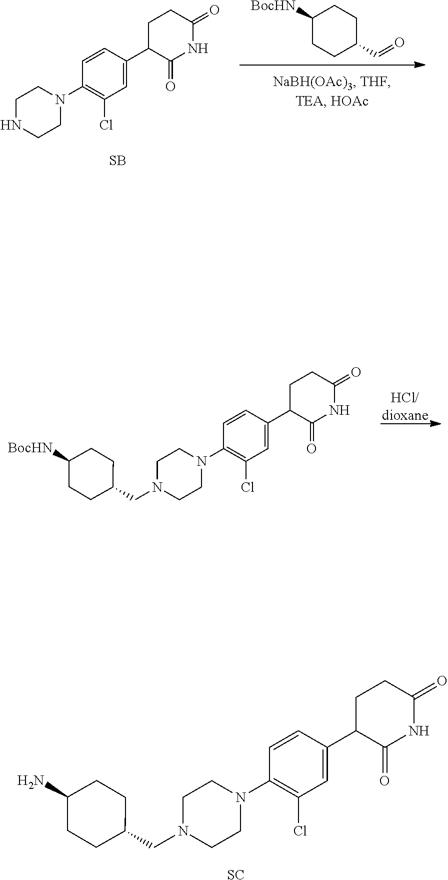

### Step 1—Tert-butyl N-[4-[[4-[2-chloro-4-(2,6-dioxo-3-piperidyl)phenyl]piperazin-1-yl]methyl]cyclohexyl]carbamate

To a solution of 3-(3-chloro-4-piperazin-1-yl-phenyl)piperidine-2,6-dione (580 mg, 1.38 mmol, TFA, Intermediate SB) in THF (5 mL) was added TEA (191 μL, 1.38 mmol) until pH=9-10. Then tert-butyl N-(4-formylcyclohexyl)carbamate (375 mg, 1.65 mmol, CAS #181308-57-6) and HOAc (78 μL, 1.38 mmol,) was added until the pH=4-5, and the mixture was stirred at 0° C. for 0.5 hr. Next, NaBH(OAc)₃ (437 mg, 2.06 mmol) was added and the mixture was stirred at 0° C. for 1 hr. On completion, the mixture was quenched with water (0.5 mL), diluted with water (10 mL), and extracted with EA (3×10 mL). Then the combined organic layers were dried over anhydrous Na₂SO₄, filtered and concentrated in vacuo to give the title compound (712 mg, 99% yield) as a light yellow oil. ¹HNMR (400 MHz, DMSO-d₆) δ 11.95 (s, 1H), 10.82 (s, 1H), 7.34 (d, J=1.6 Hz, 1H), 7.24-7.15 (m, 2H), 3.86 (dd, J=4.8, 12.0 Hz, 1H), 3.18 (d, J=6.4 Hz, 2H), 3.14-3.06 (m, 3H), 2.99-2.98 (m, 1H), 2.71-2.59 (m, 1H), 2.56 (s, 2H), 2.52-2.50 (m, 1H), 2.28-2.17 (m, 1H), 2.01-2.00 (m, 1H), 1.98 (s, 3H), 1.82-1.68 (m, 6H), 1.38-1.36 (m, 9H), 1.10-0.99 (m, 2H), 0.94-0.83 (m, 1H). LC-MS (ESI⁺) m/z 519.2 (M+H)⁺.

### Step 2—3-[4-[4-[(4-Aminocyclohexyl)methyl]piperazin-1-yl]-3-chloro-phenyl]piperidine-2,6-dione

To a solution of tert-butyl N-[4-[[4-[2-chloro-4-(2,6-dioxo-3-piperidyl)phenyl]piperazin-1-yl]methyl] cyclohexyl]carbamate (100 mg, 192 μmol) in DCM (2 mL) was added HCl/dioxane (4 M, 1 mL), then the mixture was stirred at 25° C. for 1 hr. On completion, the mixture was concentrated in vacuo to give the title compound (80.0 mg, 91% yield, HCl) as an off-white solid. LC-MS (ESI⁺) m/z 419.1 (M+H)⁺.

### Synthesis of 2,6-Dibenzyloxy-3-(4-bromophenyl)pyridine (Intermediate SD)

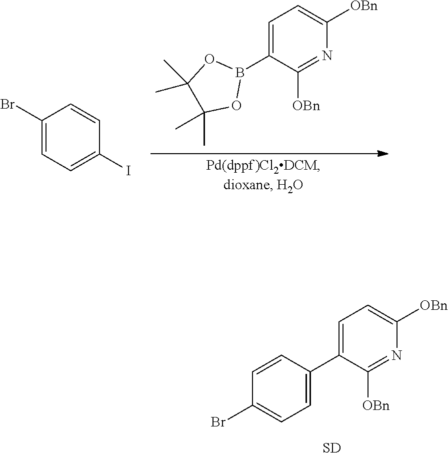

To a solution of 1-bromo-4-iodo-benzene (3 g, 10.60 mmol, CAS #589-87-7) and 2,6-dibenzyloxy-3-(4,4,5,5-tetramethyl-1,3,2-dioxaborolan-2-yl)pyridine (4.65 g, 11.1 mmol, CAS #2152673-80-6) in dioxane (25 mL) and H₂O (5 mL) was added K₂CO₃ (4.40 g, 31.8 mmol) and Pd(dppf)Cl₂·CH₂Cl₂ (865 mg, 1.06 mmol). Then the mixture was stirred at 80° C. for 2 hrs under N₂. On completion, the reaction mixture diluted with EA (200 mL), then filtered and collected the filtrate liquor. The filtrate liquor was washed with water (100 mL×2), dried over Na₂SO₄, filtered and concentrated in vacuo. The residue was purified by column chromatography (Silica gel, EA in PE, 3%, v/v) to give the title compound (2.4 g, 51% yield) as a white solid. ¹H NMR (400 MHz, DMSO-d₆) δ 7.74 (d, J=8.0 Hz, 1H), 7.60-7.54 (m, 2H), 7.53-7.48 (m, 2H), 7.46-7.41 (m, 2H), 7.40-7.25 (m, 8H), 6.56 (d, J=8.0 Hz, 1H), 5.39 (d, J=12.4 Hz, 4H)

### Synthesis of Tert-butyl N-[1-[(4-fluoro-4-piperidyl)methyl]-4-methyl-4-piperidyl]carbamate (Intermediate SE)

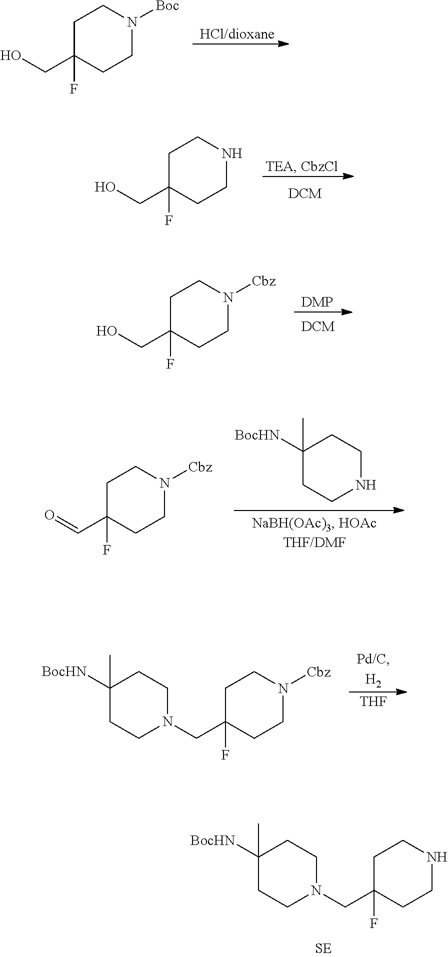

### Step 1—(4-Fluoro-4-piperidyl)methanol

A solution of tert-butyl 4-fluoro-4-(hydroxymethyl)piperidine-1-carboxylate (1 g, 4.29 mmol, CAS #614730-97-1) in HCl/dioxane (10 mL) was stirred at 25° C. for 1 hr. On completion, the reaction mixture was concentrated in vacuo to give the title compound (700 mg, 96% yield) as a white solid. ¹H NMR (400 MHz, DMSO-d₆) δ 5.57-4.35 (m, 1H), 3.51-3.38 (m, 2H), 3.20 (d, J=12.4 Hz, 2H), 2.94 (d, J=9.6 Hz, 2H), 2.02-1.79 (m, 4H).

### Step 2—Benzyl 4-fluoro-4-(hydroxymethyl)piperidine-1-carboxylate

To a solution of (4-fluoro-4-piperidyl)methanol (500 mg, 3.75 mmol) in DCM (6 mL) was added TEA (1.14 g, 11.2 mmol) and benzyl carbonochloridate (1.28 g, 7.51 mmol) at 0° C., then the reaction was stirred at 25° C. for 3 hrs. On completion, the reaction was diluted with water (30 mL) and extracted with DCM (2×40 mL). The combined organic layers were dried over Na₂SO₄, filtered and concentrated in vacuo to give a residue. The residue was purified by column chromatography (SiO₂, PE:EA=50:1 to PE:EA=5:1, PE:EA=3:1, P1:Rf=0.3) to give the title compound (700 mg, 69% yield) as a white oil. ¹H NMR (400 MHz, DMSO-d₆) δ 7.42-7.28 (m, 5H), 5.08 (s, 2H), 4.99 (s, 1H), 3.91-3.80 (m, 2H), 3.48-3.35 (m, 2H), 3.07 (s, 2H), 1.79-1.68 (m, 2H), 1.67-1.48 (m, 2H).

### Step 3—Benzyl 4-fluoro-4-formyl-piperidine-1-carboxylate

To a solution of benzyl 4-fluoro-4-(hydroxymethyl)piperidine-1-carboxylate (640 mg, 2.39 mmol) in DCM (10 mL) was added DMP (1.12 g, 2.63 mmol), then the reaction was stirred at 25° C. for 1 hr. On completion, the reaction mixture was quenched by Na₂S₂O₃ (5 mL) and NaHCO₃ (5 mL). Then the mixture was extracted with DCM (30 mL×2). The combined organic layers were washed with water (30 mL×2), dried over Na₂SO₄, filtered and concentrated in vacuo to give the title compound (600 mg, 94% yield) as a yellow oil. ¹H NMR (400 MHz, DMSO-d₆) δ 9.65 (d, J=6.4 Hz, 1H), 7.42-7.33 (m, 5H), 5.10-5.04 (m, 2H), 4.02-3.90 (m, 2H), 3.16-2.93 (m, 2H), 1.88-1.71 (m, 3H), 1.64-1.50 (m, 1H).

### Step 4—Benzyl 4-[[4-(tert-butoxycarbonylamino)-4-methyl-1-piperidyl]methyl]-4-fluoro-piperidine-1-carboxylate

To a mixture of tert-butyl N-(4-methyl-4-piperidyl)carbamate (484 mg, 2.26 mmol, CAS #163271-08-7) in DMF (4 mL) was added HOAc (271 mg, 4.52 mmol) until the pH=6 and the mixture was stirred at 25° C. for 10 min. Subsequently, benzyl 4-fluoro-4-formyl-piperidine-1-carboxylate (600 mg, 2.26 mmol) in a THF (5 mL) was added. The mixture was stirred at 25° C. for 20 min. Next, NaBH(OAc)₃ (623 mg, 2.94 mmol) was added in one portion. The resulting reaction mixture was stirred at 25° C. for 30 min. On completion, the reaction mixture was quenched with water (1 mL) and concentrated in vacuo. The residue was purified by column chromatography (SiO₂, PE:EA=5:1 to PE:EA=2:1, PE:EA=1:1, PL:Rf=0.6), then the residue was purified by prep-HPLC (column: Welch Ultimate XB-SiOH 250*50*10 um; mobile phase: [Hexane-EtOH]; B %:5%, isocratic elution mode) to give the title compound (400 mg, 38% yield) as a yellow solid. ¹H NMR (400 MHz, DMSO-d₆) δ 7.39-7.32 (m, 5H), 6.29 (s, 1H), 5.07 (s, 2H), 3.92-3.70 (m, 3H), 3.47-3.36 (m, 1H), 3.08 (s, 3H), 2.48-2.41 (m, 3H), 2.40-2.26 (m, 2H), 1.87-1.68 (m, 3H), 1.67-1.48 (m, 3H), 1.44-1.32 (m, 9H), 1.21-1.15 (m, 3H).

### Step 5—Tert-butyl N-[1-[(4-fluoro-4-piperidyl)methyl]-4-methyl-4-piperidyl]carbamate

To a solution of benzyl 4-[[4-(tert-butoxycarbonylamino)-4-methyl-1-piperidyl]methyl]-4-fluoro -piperidine-1-carboxylate (400 mg, 862 μmol) in THF (10 mL) was added Pd/C (459 mg, 431 μmol, 10 wt %) under H₂, then the reaction was stirred at 25° C. for 2 hrs under H₂. On completion, the reaction mixture was filtered and concentrated in vacuo to give the title compound (284 mg, 99% yield) as a brown solid. ¹H NMR (400 MHz, DMSO-d₆) δ 6.27 (s, 1H), 2.74-2.60 (m, 4H), 2.47-2.41 (m, 2H), 2.39-2.26 (m, 3H), 2.18 (s, 1H), 2.03-1.87 (m, 2H), 1.69-1.47 (m, 4H), 1.46 (s, 1H), 1.39-1.34 (m, 13H).

### Synthesis of 3-[4-[4-[(4-Amino-4-methyl-1-piperidyl)methyl]-4-fluoro-1-piperidyl]phenyl]piperidine-2,6-dione (Intermediate SF)

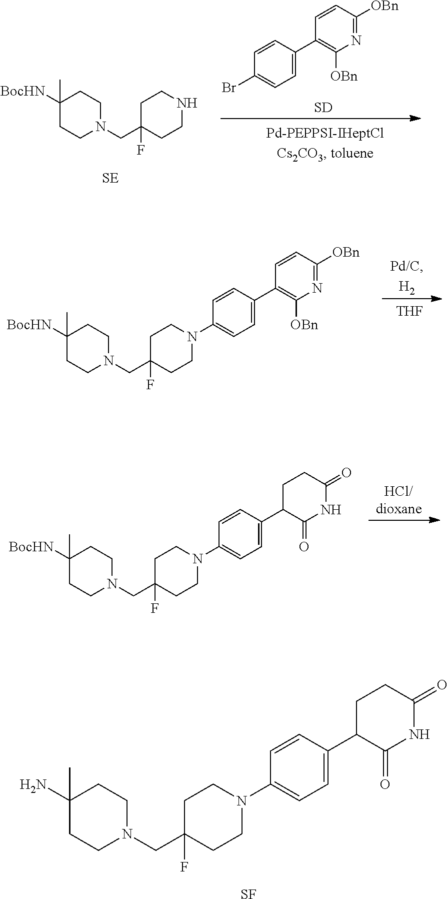

### Step 1—Tert-butyl N-[1-[[1-[4-(2,6-dibenzyloxy-3-pyridyl)phenyl]-4-fluoro-4-piperidyl]methyl]-4-methyl-4-piperidyl]carbamate

To a solution of tert-butyl N-[1-[(4-fluoro-4-piperidyl)methyl]-4-methyl-4-piperidyl]carbamate (150 mg, 455 mol, Intermediate SE) and 2,6-dibenzyloxy-3-(4-bromophenyl)pyridine (203 mg, 455 μmol, Intermediate SD) in toluene (1.5 mL) was added Cs₂CO₃ (296 mg, 910 μmol) and 1,3-bis[2,6-bis(1-propylbutyl)phenyl]-4,5-dichloro-2H-imidazol1-ium-2-ide; 3-chloropyridine; dichloropalladium (44.2 mg, 45.5 μmol). Then the reaction was stirred at 100° C. for 16 hrs under N₂. On completion, the reaction mixture was concentrated in vacuo. The residue was diluted with water (30 mL) and extracted with EA (2×30 mL). The combined organic layer was dried over Na₂SO₄, filtered and concentrated in vacuo to give a residue. The residue was purified by column chromatography (SiO₂, PE:EA=3:1 to PE:EA=1:1, PE:EA=1:1, P1:Rf=0.2) to give the title compound (182 mg, 57% yield) as a yellow solid. ¹H NMR (400 MHz, DMSO-d₆) δ 7.68 (d, J=8.0 Hz, 1H), 7.48-7.24 (m, 12H), 6.97 (d, J=8.8 Hz, 2H), 6.51 (d, J=8.0 Hz, 1H), 6.34-6.21 (m, 1H), 5.37 (d, J=17.6 Hz, 4H), 3.56-3.45 (m, 2H), 3.00 (t, J=10.8 Hz, 2H), 2.48-2.28 (m, 5H), 2.04-1.92 (m, 2H), 1.91-1.82 (m, 2H), 1.81-1.64 (m, 2H), 1.46-1.34 (m, 12H), 1.18 (s, 3H).

### Step 2—Tert-butyl N-[1-[[1-[4-(2,6-dioxo-3-piperidyl)phenyl]-4-fluoro-4-piperidyl]methyl]-4-methyl-4-piperidyl]carbamate

To a solution of tert-butyl N-[1-[[1-[4-(2,6-dibenzyloxy-3-pyridyl)phenyl]-4-fluoro-4-piperidyl]methyl]-4-methyl-4-piperidyl]carbamate (180 mg, 259 μmol) in THF (5 mL) was added Pd/C (275 mg, 259 mol, 10 wt %), then the reaction was stirred at 25° C. for 16 hrs under H₂. On completion, the reaction mixture was filtered and concentrated in vacuo to give the title compound (130 mg, 97% yield) as a yellow solid. ¹H NMR (400 MHz, DMSO-d₆) δ 10.83 (s, 1H), 7.11 (d, J=8.8 Hz, 2H), 6.98 (d, J=8.8 Hz, 2H), 6.93 (s, 1H), 3.78 (dd, J=4.8, 10.8 Hz, 1H), 3.51-3.45 (m, 2H), 3.11-2.95 (m, 2H), 2.69 (s, 2H), 2.54-2.48 (m, 2H), 2.46-2.35 (m, 2H), 2.25 (s, 2H), 2.22-1.99 (m, 4H), 1.98-1.90 (m, 2H), 1.88-1.70 (m, 2H), 1.49 (s, 2H), 1.42 (s, 9H), 1.30-1.22 (m, 3H).

### Step 3—3-[4-[4-[(4-Amino-4-methyl-1-piperidyl)methyl]-4-fluoro-1-piperidyl]phenyl]piperidine-2,6-dione

A solution of tert-butyl N-[1-[[1-[4-(2,6-dioxo-3-piperidyl)phenyl]-4-fluoro-4-piperidyl]methyl]-4-methyl -4-piperidyl]carbamate (110 mg, 212 μmol) in HCl/dioxane (3 mL) was stirred at 25° C. for 1 hr. On completion, the reaction mixture was concentrated in vacuo to give the title compound (96 mg, 99% yield) as a white solid. LC-MS (ESI⁺) m/z 417.1 (M+H)⁺.

### Synthesis of 3-[3-Fluoro-4-[(3S)-3-methylpiperazin-1-yl]phenyl]piperidine-2,6-dione (Intermediate SG)

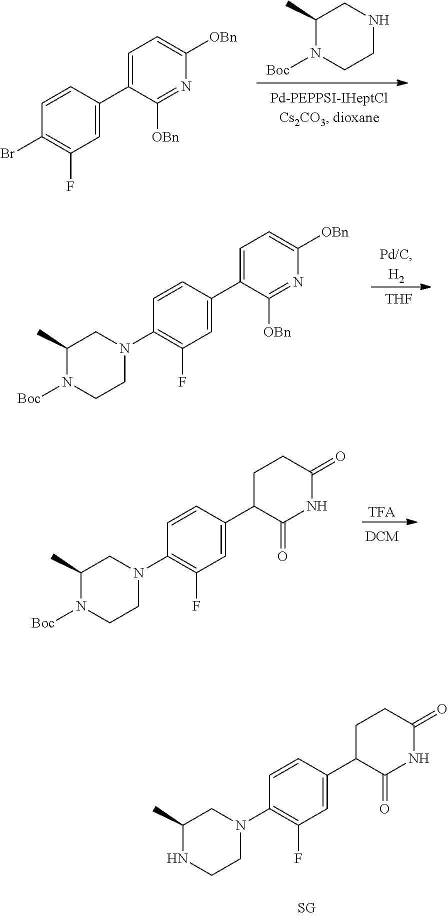 [SMILES: Fc1cc(-c2ccc(OCc3ccccc3)nc2OCc2ccccc2)ccc1Br ; C[C@H]1CNCCN1C(=O)OC(C)(C)C ; "THF" ; C[C@H]1CN(c2ccc(-c3ccc(OCc4ccccc4)nc3OCc3ccccc3)cc2F)CCN1C(=O)OC(C)(C)C ; C[C@H]1CN(c2ccc(C3CCC(=O)NC3=O)cc2F)CCN1C(=O)OC(C)(C)C ; C[C@H]1CN(c2ccc(C3CCC(=O)NC3=O)cc2F)CCN1]

### Step 1—Tert-butyl (2S)-4-[4-(2,6-dibenzyloxy-3-pyridyl)-2-fluoro-phenyl]-2-methyl-piperazine-1-carboxylate

To a solution of 2,6-dibenzyloxy-3-(4-bromo-3-fluoro-phenyl)pyridine (700 mg, 1.51 mmol, synthesized via Step 1 of Intermediate PM), tert-butyl (2S)-2-methylpiperazine-1-carboxylate (452 mg, 2.26 mmol, CAS #169447-70-5) in dioxane (10 mL) was added 1,3-bis[2,6-bis(1-propylbutyl)phenyl]-4,5-dichloro-2H-imidazol-1-ium-2-ide 3-chloropyridine; dichloropalladium (146 mg, 150 μmol), Cs₂CO₃ (1.47 g, 4.52 mmol), and 4A molecular sieves (200 mg) and the mixture was purged with N₂ for 3 times. Then the mixture was stirred at 110° C. for 12 hrs under N₂ atmosphere. On completion, the residue was diluted with water (30 mL), then extracted with EA (3×30 mL). The combined organic layers were dried over Na₂SO₄, filtered and concentrated in vacuo. The residue was purified by column chromatography (SiO₂, PE:EA=50:1 to PE:EA=15:1, PE:EA=5:1, PL:Rf=0.6) to give the title compound (420 mg, 47% yield) as red solid. ¹H NMR (400 MHz, DMSO-d₆) δ 7.75 (d, J=8.0 Hz, 1H), 7.47-7.27 (m, 12H), 7.02 (t, J=8.8 Hz, 1H), 6.54 (d, J=8.0 Hz, 1H), 5.39 (d, J=16.4 Hz, 4H), 4.20 (s, 1H), 3.81 (d, J=13.2 Hz, 1H), 3.30-3.25 (m, 1H), 3.24-3.10 (m, 2H), 2.78-2.75 (m, 1H), 2.71-2.63 (m, 1H), 1.42 (s, 9H), 1.25 (d, J=6.4 Hz, 3H); LC-MS (ESI⁺) m/z 584.3 (M+H)⁺.

### Step 2—Tert-butyl (2S)-4-[4-(2,6-dioxo-3-piperidyl)-2-fluoro-phenyl]-2-methyl-piperazine-1-carboxylate

To a solution of tert-butyl (2S)-4-[4-(2,6-dibenzyloxy-3-pyridyl)-2-fluoro-phenyl]-2-methyl-piperazine-1-carboxylate (970 mg, 1.66 mmol) in THF (10 mL) was added Pd/C (500 mg, 469 μmol, 10 wt %) under Ar. The suspension was degassed under vacuum and purged with H₂ several times. The mixture was stirred under H₂ (15 psi) at 25° C. for 12 hrs. On completion, the reaction mixture was filtered and concentrated in vacuo to give the title compound (660 mg, 97% yield) as gray solid. ¹H NMR (400 MHz, DMSO-d₆) δ 10.81 (s, 1H), 7.04 (d, J=14.0 Hz, 1H), 6.96 (d, J=4.8 Hz, 2H), 4.20 (s, 1H), 3.82-3.78 (m, 2H), 3.24 (d, J=11.2 Hz, 1H), 3.21-3.09 (m, 2H), 2.75 (d, J=10.0 Hz, 1H), 2.70-2.59 (m, 2H), 2.53-2.51 (m, 1H), 2.24-2.14 (m, 1H), 2.02-1.96 (m, 1H), 1.42 (s, 9H), 1.25 (d, J=6.8 Hz, 3H); LC-MS (ESI⁺) m/z 406.0 (M+H)⁺.

### Step 3—3-[3-Fluoro-4-[(3S)-3-methylpiperazin-1-yl]phenyl]piperidine-2,6-dione

To a solution of tert-butyl (2S)-4-[4-(2,6-dioxo-3-piperidyl)-2-fluoro-phenyl]-2-methyl-piperazine-1-carboxylate (300 mg, 739 μmol) in DCM (3 mL) was added TFA (1.54 g, 13.4 mmol, 1 mL), and the mixture was stirred at 25° C. for 0.5 hr. On completion, the reaction mixture was concentrated in vacuo to give the title compound (310 mg, 99% yield, TFA) as yellow oil. LC-MS (ESI⁺) m/z 306.0 (M+H)⁺.

### Synthesis of 3-[4-[(3S)-4-[(4-aminocyclohexyl)methyl]-3-methyl-piperazin-1-yl]-3-fluoro-phenyl]piperidine-2,6-dione (Intermediate SH)

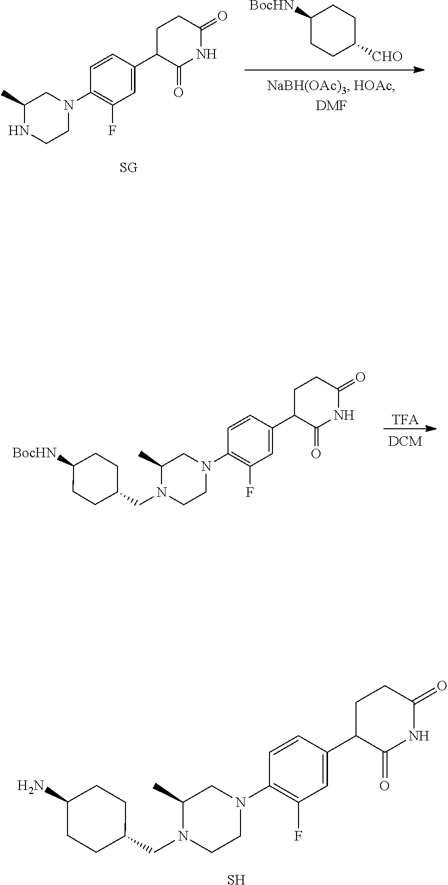

### Step 1—Tert-butyl N-[4-[[(2S)-4-[4-(2,6-dioxo-3-piperidyl)-2-fluoro-phenyl]-2-methyl-piperazin-1-yl]methyl]cyclohexyl]carbamate

To a solution of 3-[3-fluoro-4-[(3S)-3-methylpiperazin-1-yl]phenyl]piperidine-2,6-dione (310 mg, 739 μmol, TFA, Intermediate SG) in DMF (3 mL) was added TEA (74.8 mg, 739 mol, 102 μL) until the pH=8-10. Then tert-butyl N-(4-formylcyclohexyl)carbamate (184 mg, 813 μmol, CAS #181308-57-6) and HOAc (44.3 mg, 739 μmol, 42.3 μL) was added to the mixture and the mixture was stirred at −10° C. for 0.5 hr. Next, NaBH(OAc)₃ (313 mg, 1.48 mmol) was added and the mixture was stirred at −10° C. for 1 hr. On completion, the reaction mixture was quenched with water (0.5 mL) and concentrated in vacuo. The residue was purified by prep-HPLC (column: Phenomenex luna C18 150*25 mm*10 um; mobile phase: [water (FA)-ACN]; gradient: 11%-41% B over 10 min) to give the title compound (300 mg, 78% yield) as white solid. ¹H NMR (400 MHz, DMSO-d₆) δ 10.83 (s, 1H), 7.14-6.99 (m, 3H), 6.78 (d, J=7.2 Hz, 1H), 3.84-3.80 (m, 1H), 3.68 (d, J=8.8 Hz, 1H), 3.47 (d, J=9.6 Hz, 2H), 3.25-3.08 (m, 4H), 2.94 (d, J=8.4 Hz, 2H), 2.71-2.61 (m, 1H), 2.24-2.16 (m, 1H), 2.01-1.96 (m, 1H), 1.93-1.58 (m, 6H), 1.37 (s, 12H), 1.24-1.10 (m, 3H), 1.04 (s, 2H); LC-MS (ESI⁺) m/z 517.3 (M+H)⁺.

### Step 2—3-[4-[(3S)-4-[(4-aminocyclohexyl)methyl]-3-methyl-piperazin-1-yl]-3-fluoro-phenyl] piperidine-2,6-dione

To a solution of tert-butyl N-[4-[[(2S)-4-[4-(2,6-dioxo-3-piperidyl)-2-fluoro-phenyl]-2-methyl-piperazin-1-yl]methyl]cyclohexyl]carbamate (100 mg, 193 μmol) in DCM (1 mL) was added TFA (575 mg, 5.05 mmol, 375 μL), then the mixture was stirred at 25° C. for 0.5 hr. On completion, the reaction mixture was concentrated in vacuo to give the title compound (102 mg, 99% yield, TFA) as red oil. LC-MS (ESI⁺) m/z 417.2 (M+H)⁺.

### Synthesis of 3-(3-Methyl-4-piperazin-1-yl-phenyl)piperidine-2,6-dione (Intermediate SI)

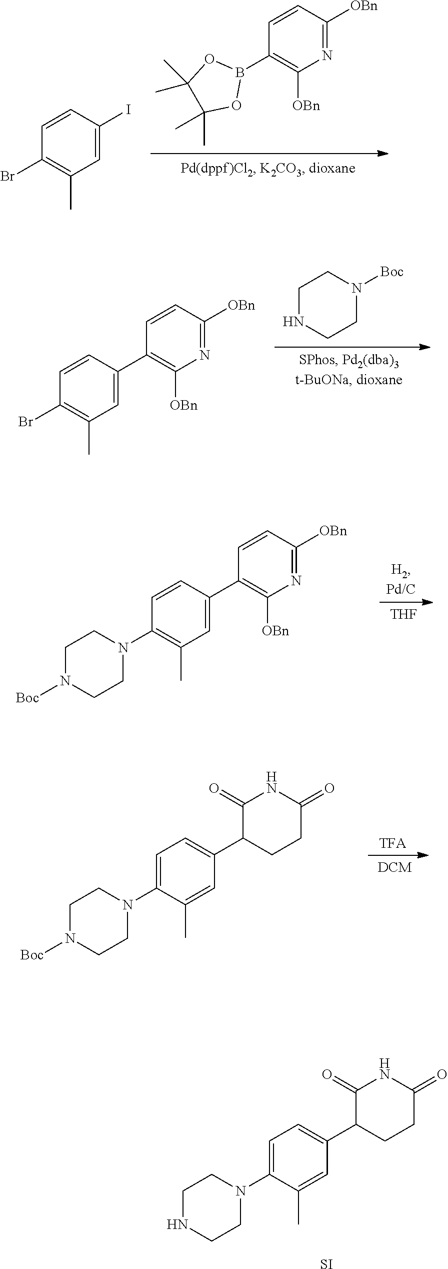

### Step 1—2,6-Dibenzyloxy-3-(4-bromo-3-methyl-phenyl)pyridine

To a solution of 1-bromo-4-iodo-2-methyl-benzene (1.71 g, 5.75 mmol, CAS #202865-85-8), 2,6-dibenzyloxy -3-(4, 4, 5, 5-tetramethyl-1,3,2-dioxaborolan-2-yl)pyridine (2.00 g, 4.79 mmol, CAS#2152673-80-6) in dioxane (12 mL) and H₂O (1 mL) was added Pd(dppf)Cl₂ (351 mg, 479 mol) and K₂CO₃ (1.32 g, 9.59 mmol). The mixture was then stirred at 80° C. for 12 hrs. On completion, the reaction mixture was concentrated in vacuo. The crude product was purified by reversed-phase HPLC (0.1% TFA condition) to give the title compound (2.00 g, 82% yield) as a white gum. ¹H NMR (400 MHz, DMSO-d₆) δ 7.74 (s, 1H), 7.64 (s, 1H), 7.60-7.52 (m, 2H), 7.44 (s, 3H), 7.42-7.29 (m, 9H), 6.56 (d, J=8.0 Hz, 1H), 5.41 (s, 2H), 2.35 (s, 3H).

### Step 2—Tert-butyl-4-[4-(2,6-dibenzyloxy-3-pyridyl)-2-methyl-phenyl]piperazine-1-carboxylate

To a solution of 2,6-dibenzyloxy-3-(4-bromo-3-methyl-phenyl)pyridine (2 g, 4.34 mmol) and tert-butyl piperazine-1-carboxylate (1.21 g, 6.52 mmol, CAS #143238-38-4) in dioxane (20 mL) was added SPhos (178 mg, 434 μmol), Pd₂(dba)₃ (398 mg, 434 μmol) and t-BuONa (1.25 g, 13.0 mmol). The mixture was degassed and purged with N₂ for 3 times. The mixture was then stirred at 100° C. for 2 hrs under N₂ atmosphere. On completion, the reaction mixture was concentrated in vacuo. The residue was purified by flash silica gel chromatography (ISCO®; 12 g SepaFlash® Silica Flash Column, Eluent of 0˜10% Ethylacetate/Petroleum ether gradient @ 30 mL/min) and purified by reversed-phase HPLC again (0.1% FA condition) to give the title compound (1.50 g, 55% yield) as a brown gum. ¹H NMR (400 MHz, DMSO-d₆) δ 7.69 (s, 1H), 7.49-7.29 (m, 12H), 7.08-6.98 (m, 1H), 6.53 (d, J=8.0 Hz, 1H), 5.38 (d, J=11.6 Hz, 4H), 3.47 (s, 4H), 2.80 (s, 4H), 2.27 (s, 2H), 1.99 (s, 1H), 1.43 (s, 9H).

### Step 3—Tert-butyl-4-[4-(2,6-dioxo-3-piperidyl)-2-methyl-phenyl]piperazine-1-carboxylate

To a solution of tert-butyl 4-[4-(2,6-dibenzyloxy-3-pyridyl)-2-methyl-phenyl]piperazine-1-carboxylate (1.50 g, 2.65 mmol) in THF (20 mL) was added Pd/C (398 mg, 374 μmol, 10 wt %) under N₂ atmosphere. The suspension was degassed and purged with H₂ three times. The mixture was stirred under H₂ (15 psi) at 25° C. for 12 hrs. On completion, the reaction mixture was filtered and the filtrated was concentrated in vacuo to give a residue. The residue was purified by flash silica gel chromatography (ISCO®; 12 g SepaFlash® Silica Flash Column, Eluent of 0˜60% Ethyl acetate/Petroleum ethergradient @ 30 mL/min) to give the title compound (1.00 g, 88% yield) as a white solid. ¹H NMR (400 MHz, DMSO-d₆) δ 10.80 (s, 1H), 7.02 (s, 1H), 6.98 (s, 2H), 3.75 (dd, J=4.8, 11.2 Hz, 1H), 3.46 (s, 4H), 2.77 (s, 4H), 2.70-2.59 (m, 1H), 2.45 (s, 1H), 2.25 (s, 3H), 2.20-2.09 (m, 1H), 2.00 (s, 1H), 1.43 (s, 9H).

### Step 4—3-(3-Methyl-4-piperazin-1-yl-phenyl)piperidine-2,6-dione

To a solution of tert-butyl 4-[4-(2,6-dioxo-3-piperidyl)-2-methyl-phenyl]piperazine-1-carboxylate (200 mg, 516 μmol) in DCM (4 mL) was added TFA (3.07 g, 2 mL). The mixture was then stirred at 25° C. for 0.5 hr. On completion, the reaction mixture was concentrated in vacuo to give the title compound (145 mg, 70% yield, TFA salt) as a yellow oil. LC-MS (ESI⁺) m/z 288.1 (M+H).

### Synthesis of 3-[4-[4-[(4-Aminocyclohexyl)methyl]piperazin-1-yl]-3-methyl-phenyl]piperidine-2,6-dione (Intermediate SJ)

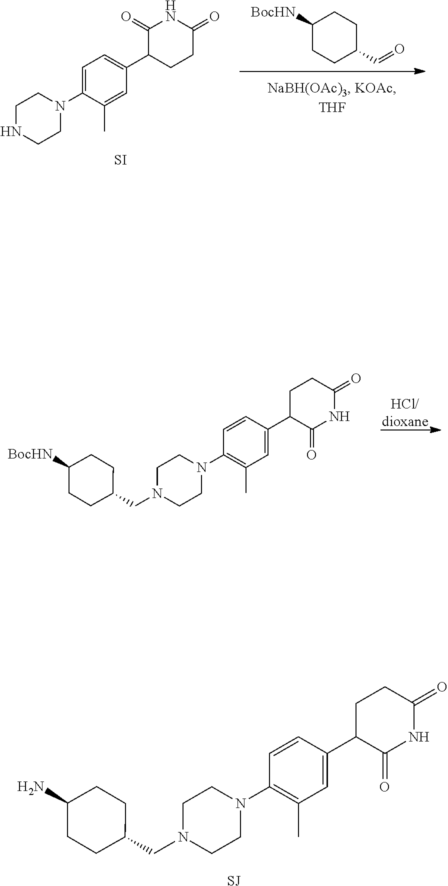

### Step 1—Tert-butyl-N-[4-[[4-[4-(2,6-dioxo-3-piperidyl)-2-methyl-phenyl]piperazin-1-yl]methyl]cyclohexyl]carbamate

To a solution of 3-(3-methyl-4-piperazin-1-yl-phenyl)piperidine-2,6-dione (145 mg, 505 μmol, Intermediate SI), tert-butyl N-(4-formylcyclohexyl)carbamate (138 mg, 606 μmol, CAS #181308-57-6) in THF (2 mL) and DMF (1 mL) was added KOAc (149 mg, 1.51 mmol) and NaBH(OAc)₃ (214 mg, 1.01 mmol). The mixture was then stirred at −10° C. for 1 hr. On completion, the reaction mixture was concentrated in vacuo. The crude product was purified by reversed-phase HPLC (0.1% FA condition) to give the title compound (200 mg, 72% yield) as a white solid. 1H NMR (400 MHz, DMSO-d₆) δ 10.80 (s, 1H), 7.13-6.90 (m, 3H), 6.74 (d, J=7.6 Hz, 1H), 3.75 (dd, J=4.8, 11.2 Hz, 1H), 3.34 (s, 3H), 3.22-3.12 (m, 1H), 2.93 (s, 4H), 2.86-2.72 (m, 2H), 2.72-2.58 (m, 2H), 2.46 (d, J=4.4 Hz, 2H), 2.33 (s, 1H), 2.22-2.07 (m, 2H), 2.02 (d, J=4.8 Hz, 1H), 1.79 (d, J=11.2 Hz, 4H), 1.53 (s, 1H), 1.38 (s, 9H), 1.15 (d, J=12.0 Hz, 2H), 0.94 (d, J=11.2 Hz, 2H).

### Step 2—3-[4-[4-[(4-Aminocyclohexyl)methyl]piperazin-1-yl]-3-methyl-phenyl]piperidine-2,6-dione

To a solution of tert-butyl N-[4-[[4-[4-(2, 6-dioxo-3-piperidyl)-2-methyl-phenyl] piperazin -1-yl]methyl]cyclohexyl]carbamate (150 mg, 301 μmol) in DCM (1 mL) was added HCl/dioxane (1 mL). Then the mixture was stirred at 25° C. for 1 hr. On completion, the reaction mixture was concentrated in vacuo to give the title compound (120 mg, 92% yield, HCl salt) as a white solid. LC-MS (ESI⁺) m/z 399.2 (M+H).

### Synthesis of 3-(3-Fluoro-2-methyl-4-piperazin-1-yl-phenyl)piperidine-2,6-dione (Intermediate SK)

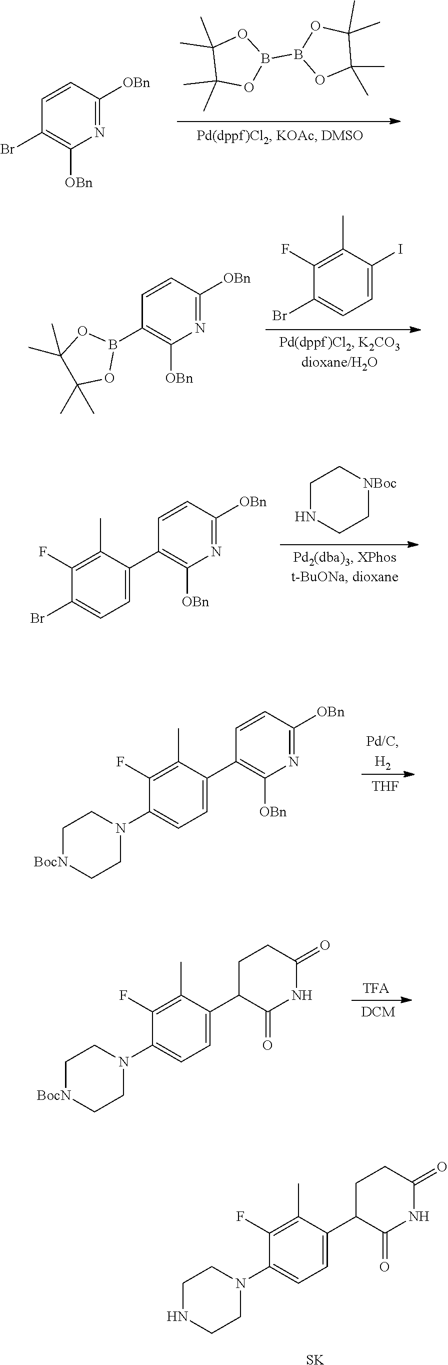

### Step 1—2,6-Dibenzyloxy-3-(4,4,5,5-tetramethyl-1,3,2-dioxaborolan-2-yl)pyridine

To a solution of 2,6-dibenzyloxy-3-bromo-pyridine (5 g, 13.5 mmol, CAS #16727-47-2) and 4,4,5,5-tetramethyl-2-(4,4,5,5-tetramethyl-1,3,2-dioxaborolan-2-yl)-1,3,2-dioxaborolane (10.2 g, 40.5 mmol, CAS #73183-34-3) in DMSO (50 mL) was added KOAc (3.98 g, 40.5 mmol) and Pd(dppf)Cl₂·CH₂Cl₂ (1.10 g, 1.35 mmol). The reaction mixture was then stirred at 100° C. for 16 hrs under N₂. On completion, the mixture was diluted with EA (200 mL) and washed with water (100 mL×3). The organic layer was dried over Na₂SO₄, filtered and concentrated in vacuo. The residue was purified by column chromatography (SiO₂, PE:EA=1:0 to 10:1) to give the title compound (4 g, 70% yield) as green oil. LC-MS (ESI⁺) m/z 418.1 (M+H)⁺.

### Step 2—2,6-Dibenzyloxy-3-(4-bromo-3-fluoro-2-methyl-phenyl)pyridine

To a solution of 1-bromo-2-fluoro-4-iodo-3-methyl-benzene (1.9 g, 6.03 mmol, CAS #1000576-29-3) and 2,6-dibenzyloxy-3-(4,4,5,5-tetramethyl-1,3,2-dioxaborolan-2-yl)pyridine (2.01 g, 4.83 mmol) in dioxane (20 mL) and H₂O (4 mL) was added Pd(dppf)Cl₂·CH₂Cl₂ (492 mg, 603 μmol) and K₂CO₃ (2.50 g, 18.1 mmol). The reaction mixture was then stirred at 80° C. for 2 hrs under N₂. On completion, the mixture was diluted with EA (80 mL) and washed with water (40 mL×2). The organic layer was dried over Na₂SO₄, filtered and concentrated in vacuo. The residue was purified by column chromatography (SiO₂, PE:EA=1:0 to 20:1) to give the title compound (1.85 g, 64% yield) as yellow oil. ¹H NMR (400 MHz, DMSO-d₆) δ 7.60-7.50 (m, 2H), 7.47-7.28 (m, 10H), 6.98 (d, J=8.0 Hz, 1H), 6.55 (d, J=8.0 Hz, 1H), 5.37 (d, J=6.0 Hz, 4H), 2.00 (d, J=2.8 Hz, 3H).

### Step 3—Tert-butyl 4-[4-(2,6-dibenzyloxy-3-pyridyl)-2-fluoro-3-methyl-phenyl]piperazine-1-carboxylate

To a solution of 2,6-dibenzyloxy-3-(4-bromo-3-fluoro-2-methyl-phenyl)pyridine (1.73 g, 3.62 mmol) and tert-butyl piperazine-1-carboxylate (1.01 g, 5.42 mmol, CAS #57260-71-6) in dioxane (20 mL) was added t-BuONa (1.04 g, 10.8 mmol), Pd₂(dba)₃ (331 mg, 361 μmol) and XPhos (172 mg, 361 μmol). The reaction was then stirred at 110° C. for 3 hrs under N₂. On completion, the mixture was diluted with EA (80 mL) and washed with water (40 mL×2). The organic layer was dried over Na₂SO₄, filtered and concentrated in vacuo. The residue was purified by column chromatography (SiO₂, PE:EA=1:0 to 10:1) to give the title compound (1 g, 47% yield) as yellow oil. ¹H NMR (400 MHz, DMSO-d₆) δ 7.47-7.29 (m, 11H), 6.90 (s, 2H), 6.52 (d, J=8.0 Hz, 1H), 5.35 (s, 4H), 3.47 (s, 4H), 3.01-2.91 (m, 4H), 1.94 (d, J=2.8 Hz, 3H), 1.42 (s, 9H).

### Step 4—Tert-butyl 4-[4-(2,6-dioxo-3-piperidyl)-2-fluoro-3-methyl-phenyl]piperazine-1-carboxylate

To a solution of tert-butyl 4-[4-(2,6-dibenzyloxy-3-pyridyl)-2-fluoro-3-methyl-phenyl]piperazine-1-carboxylate (900 mg, 1.54 mmol) in THF (20 mL) was added Pd/C (500 mg, 469 μmol, 10 wt %) under N₂. The suspension was degassed under vacuum and purged with H₂ several times. The mixture was then stirred under H₂ (15 psi) at 25° C. for 24 hrs. On completion, the mixture was filtered and the filtrate was concentrated in vacuo to give the title compound (400 mg, 63% yield) as purple solid. LC-MS (ESI⁺) m z 406.1 (M+H)⁺.

### Step 5—3-(3-Fluoro-2-methyl-4-piperazin-1-yl-phenyl)piperidine-2,6-dione

To a solution of tert-butyl4-[4-(2,6-dioxo-3-piperidyl)-2-fluoro-3-methyl-phenyl] piperazine-1-carboxylate (180 mg, 443 μmol) in DCM (3 mL) was added TFA (1.54 g, 13.4 mmol, 1 mL). The reaction mixture was then stirred at 25° C. for 0.5 hr. On completion, the mixture was concentrated in vacuo to give the title compound (186 mg, 99% yield, TFA) as brown oil. LC-MS (ESI⁺) m/z 306.0 (M+H)⁺.

### Synthesis of 3-[4-[4-[(4-Aminocyclohexyl)methyl]piperazin-1-yl]-3-fluoro-2-methyl-phenyl]piperidine-2,6-dione (Intermediate SL)

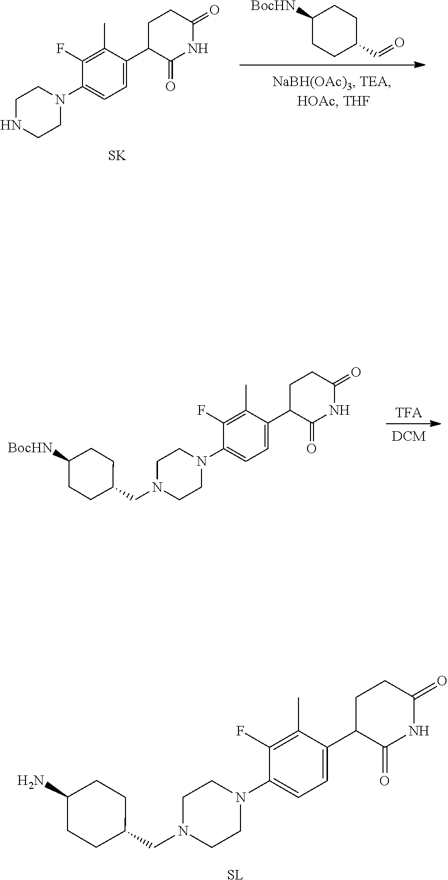

### Step 1—Tert-butyl N-[4-[[4-[4-(2,6-dioxo-3-piperidyl)-2-fluoro-3-methyl-phenyl]piperazin-1-yl]methyl]cyclohexyl]carbamate

To a solution of 3-(3-fluoro-2-methyl-4-piperazin-1-yl-phenyl)piperidine-2,6-dione (186 mg, 443 mol, TFA, Intermediate SK) in THF (3 mL) was added TEA (44.8 mg, 443 μmol, 61.7 μL) until the pH=8 and the mixture was stirred for 5 mins. Then, HOAc (53.2 mg, 887 mol, 50.7 μL) was added until the mixture pH=6. Next, tert-butyl N-(4-formylcyclohexyl)carbamate (100 mg, 443 μmol, CAS #181308-57-6) was added and the mixture was stirred at −10° C. for 0.5 hr. Finally, NaBH(OAc)₃ (141 mg, 665 mol) was added and the mixture was stirred at −10° C. for 0.5 hr. On completion, the mixture was quenched with H₂O (0.2 mL) and concentrated in vacuo. The residue was purified by prep-HPLC (column: Phenomenex luna C18 150*25 mm*10 um; mobile phase: [water (FA)-ACN]; gradient: 17%-37% B over 10 min) to give the title compound (180 mg, 78% yield) as yellow solid. ¹H NMR (400 MHz, DMSO-d₆) δ 10.83 (s, 1H), 7.02-6.62 (m, 3H), 4.02 (dd, J=3.6, 10.8 Hz, 1H), 3.58 (dd, J=3.2, 5.6 Hz, 1H), 3.49-3.41 (m, 1H), 3.26-2.83 (m, 7H), 2.77-2.69 (m, 1H), 2.41 (d, J=7.6 Hz, 1H), 2.24-2.16 (m, 1H), 2.13 (s, 3H), 2.07 (s, 2H), 1.99-1.90 (m, 1H), 1.78 (d, J=10.0 Hz, 5H), 1.37 (s, 9H), 1.24-0.88 (m, 4H).

### Step 2—3-[4-[4-[(4-Aminocyclohexyl)methyl]piperazin-1-yl]-3-fluoro-2-methyl-phenyl]piperidine-2,6-dione

To a solution of tert-butyl N-[4-[[4-[4-(2,6-dioxo-3-piperidyl)-2-fluoro-3-methyl-phenyl]piperazin-1-yl]methyl]cyclohexyl]carbamate (90 mg, 174 μmol) in DCM (1.5 mL) was added TFA (767 mg, 6.73 mmol, 0.5 mL). The reaction mixture was then stirred at 25° C. for 1.5 hrs. On completion, the mixture was concentrated in vacuo to give the title compound (92 mg, 99% yield, TFA) as colorless oil. LC-MS (ESI⁺) m/z 417.1 (M+H)⁺.

### Synthesis of 3-[5-[4-[(4-Aminocyclohexyl)methyl]piperazin-1-yl]-4-fluoro-3-methyl-2-oxo-benzimidazol-1-yl]piperidine-2,6-dione (Intermediate SM)

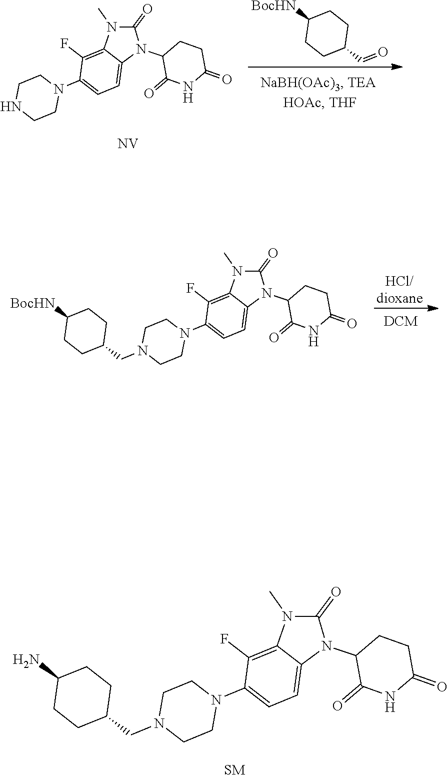

### Step 1—Tert-butyl N-[4-[[4-[1-(2,6-dioxo-3-piperidyl)-4-fluoro-3-methyl-2-oxo-benzimidazol-5-yl]piperazin-1-yl]methyl]cyclohexyl]carbamate

To a solution of 3-(4-fluoro-3-methyl-2-oxo-5-piperazin-1-yl-benzimidazol-1-yl)piperidine-2,6-dione (130 mg, 359 μmol, Intermediate NV) in DMF (0.5 mL) and THF (0.5 mL) was added NaBH(OAc)₃ (114 mg, 539 μmol), tert-butyl N-(4-formylcyclohexyl)carbamate (81.7 mg, 359 μmol, CAS #181308-57-6) and KOAc (353 mg, 3.60 mmol). The mixture was then stirred at 25° C. for 1 hr. On completion, the reaction mixture was diluted with water (100 mL) and extracted with ethyl acetate (100 mL×3). The combined organic layers were washed by brine (100 mL×3), dried over anhydrous sodium sulfate, filtered and concentrated under reduced pressure. The crude product was purified by reversed-phase HPLC (0.1% FA condition) to give title compound (100 mg, 47% yield) as a white solid. ¹H NMR (400 MHz, DMSO-d₆) δ 11.10 (s, 1H), 6.87 (d, J=8.0 Hz, 1H), 6.80-6.64 (m, 2H), 5.40-5.27 (m, 1H), 3.47 (s, 3H), 3.40-3.39 (m, 1H), 3.21-3.12 (m, 2H), 3.10-2.94 (m, 4H), 2.94-2.82 (m, 2H), 2.71-2.58 (m, 4H), 2.04-1.96 (m, 1H), 1.84-1.71 (m, 4H), 1.66-1.40 (m, 2H), 1.37 (s, 10H), 1.20-1.08 (m, 2H), 1.01-0.82 (m, 2H).

### Step 2—3-[5-[4-[(4-Aminocyclohexyl)methyl]piperazin-1-yl]-4-fluoro-3-methyl-2-oxo-benzimidazol -1-yl]piperidine-2,6-dione

To a solution of tert-butyl N-[4-[[4-[1-(2,6-dioxo-3-piperidyl)-4-fluoro-3-methyl-2-oxo-benzimidazol-5-yl]piperazin-1-yl]methyl]cyclohexyl]carbamate (50.0 mg, 87.3 μmol) in DCM (1 mL) was added HCl/dioxane (4 M, 0.5 mL). The mixture was then stirred at 25° C. for 1 hr. On completion, the reaction mixture was concentrated under reduced pressure to give title compound (40 mg, 96% yield) as yellow oil. LC-MS (ESI⁺) m/z 473.2 (M+H)⁺.

### Synthesis of 3-[4-[4-[(4-Amino-4-methyl-cyclohexyl)methyl]piperazin-1-yl]-3-fluoro-phenyl]piperidine-2,6-dione (Intermediate SN)

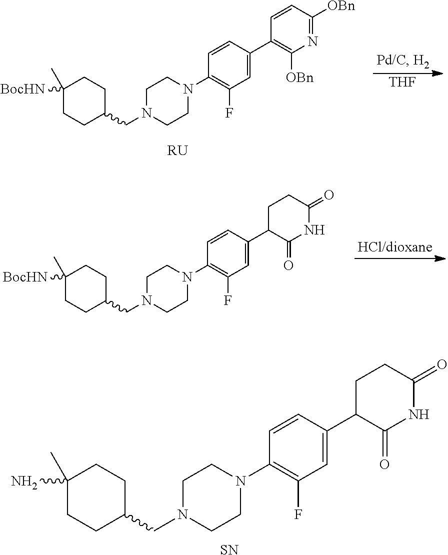

### Step 1—Tert-butyl N-[4-[[4-[4-(2,6-dioxo-3-piperidyl)-2-fluoro-phenyl]piperazin-1-yl]methyl]-1-methyl-cyclohexyl]carbamate

To a solution of tert-butyl N-[4-[[4-[4-(2,6-dibenzyloxy-3-pyridyl)-2-fluoro-phenyl]piperazin-1-yl] methyl]-1-methyl-cyclohexyl]carbamate (600 mg, 863 μmol, Intermediate RU) in THF (20 mL) was added Pd/C (600 mg, 563 μmol, 10 wt %) under Ar. The suspension was degassed under vacuum and purged with H₂ several times. The mixture was then stirred under H₂ (15 psi) at 25° C. for 16 hrs. On completion, the reaction mixture was filtered and the filtrated was concentrated in vacuo to give the title compound (440 mg, 98% yield) as gray solid. ¹H NMR (400 MHz, DMSO-d₆) δ 10.80 (s, 1H), 7.05-6.92 (m, 3H), 6.17-6.07 (m, 1H), 3.84-3.75 (m, J=4.8, 11.6 Hz, 1H), 3.29 (s, 1H), 2.98 (s, 4H), 2.68-2.60 (m, 1H), 2.46 (s, 4H), 2.24-2.16 (m, 1H), 2.12 (d, J=6.8 Hz, 4H), 2.04-1.95 (m, 1H), 1.49 (d, J=11.6 Hz, 2H), 1.43 (s, 1H), 1.37 (s, 9H), 1.17 (s, 3H), 1.12-0.98 (m, 4H); LC-MS (ESI⁺) m/z 517.3 (M+H)⁺.

### Step 2—3-[4-[4-[(4-amino-4-methyl-cyclohexyl)methyl]piperazin-1-yl]-3-fluoro-phenyl]piperidine-2,6-dione

To a solution of tert-butyl N-[4-[[4-[4-(2,6-dioxo-3-piperidyl)-2-fluoro-phenyl]piperazin-1-yl]methyl]-1-methyl-cyclohexyl]carbamate (100 mg, 193 μmol) in DCM (0.5 mL) was added HCl/dioxane (4 M, 2 mL). The reaction was then stirred at 40° C. for 0.5 hr. On completion, the reaction was concentrated in vacuo to give the title compound (87 mg, 99% yield, HCl) as white solid. LC-MS (ESI⁺) m/z 417.0 (M+H)⁺.

### Synthesis of Tert-butyl N-[4-fluoro-4-(piperazin-1-ylmethyl)cyclohexyl]carbamate (Intermediate SO)

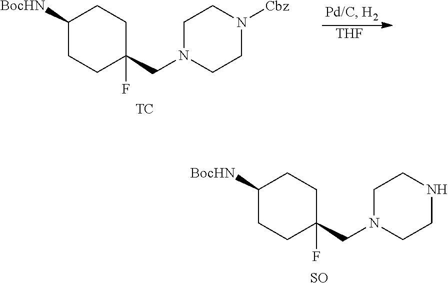

To a solution of benzyl 4-[[4-(tert-butoxycarbonylamino)-1-fluoro-cyclohexyl]methyl]piperazine-1-carboxylate (210 mg, 467 μmol, Intermediate TC) in THF (20 mL) was added Pd/C (210 mg, 197 μmol, 10 wt %) under N₂. The suspension was degassed under vacuum and purged with H₂ (941 μg, 467 mol) several times. The mixture was stirred under H₂ (941 μg, 467 μmol) (15 psi) at 20° C. for 1 h. On completion, the reaction was filtered and the filtrate was concentrated in vacuo to give the title compound (140 mg, 95% yield) as yellow solid. ¹H NMR (400 MHz, CDCl₃) δ 4.36 (s, 1H), 3.71-3.64 (m, 1H), 3.43-3.29 (m, 1H), 2.85-2.76 (m, 4H), 2.47-2.39 (m, 4H), 2.20 (s, 2H), 1.95 (dd, J=8.4, 10.8 Hz, 2H), 1.79-1.75 (m, 4H), 1.37 (s, 9H), 1.36 (s, 2H).

### Synthesis of 3-[4-[4-[(4-Amino-1-fluoro-cyclohexyl)methyl]piperazin-1-yl]-3-fluoro-phenyl]piperidine-2,6-dione (Intermediate SP)

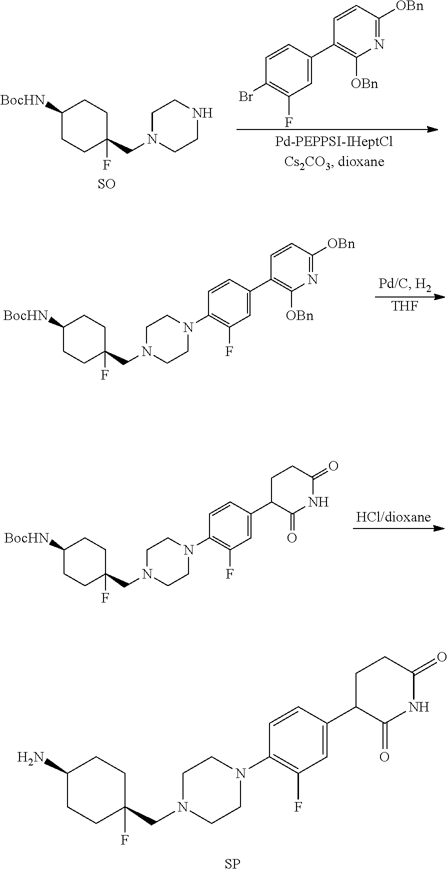

### Step 1—Tert-butyl N-[4-[[4-[4-(2,6-dibenzyloxy-3-pyridyl)-2-fluoro-phenyl]piperazin-1-yl]methyl]-4-fluoro-cyclohexyl]carbamate

To a solution tert-butyl N-[4-fluoro-4-(piperazin-1-ylmethyl)cyclohexyl]carbamate (120 mg, 380 mol, Intermediate SO) in 2,6-dibenzyloxy-3-(4-bromo-3-fluoro-phenyl)pyridine (160 mg, 345 μmol, synthesized via Step 1 of Intermediate PM) in dioxane (1.5 mL) was added Cs₂CO₃ (338 mg, 1.04 mmol) and 1,3-bis[2,6-bis(1-propylbutyl)phenyl]-4,5-dichloro-2H-imidazol-1-ium-2-ide 3-chloropyridine dichloropalladium (33.6 mg, 34.5 μmol). Then the mixture was stirred at 100° C. for 2 hrs under N₂. On completion, the reaction was filtered and the filtrate was concentrated in vacuo. The residue was purified by column chromatography (SiO₂, PE:EA=20:1 to 2:1 PE:EA=2:1, Rf=0.3) to give the title compound (160 mg, 66% yield) as yellow solid. ¹H NMR (400 MHz, DMSO-d₆) δ 7.85-7.77 (m, 1H), 7.50-7.36 (m, 12H), 7.08 (t, J=8.8 Hz, 1H), 6.85 (d, J=8.0 Hz, 1H), 6.59 (d, J=8.0 Hz, 1H), 5.47 (s, 2H), 5.42 (s, 2H), 3.34-3.24 (m, 1H), 3.08 (s, 4H), 2.68 (s, 4H), 2.05-1.94 (m, 2H), 1.68 (d, J=1.6 Hz, 2H), 1.57-1.53 (m, 1H), 1.49-1.45 (m, 2H), 1.44 (s, 9H), 0.94-0.74 (m, 3H).

### Step 2—Tert-butyl N-[4-[[4-[4-(2,6-dioxo-3-piperidyl)-2-fluoro-phenyl]piperazin-1-yl]methyl]-4-fluoro-cyclohexyl]carbamate

To a solution of tert-butyl N-[4-[[4-[4-(2,6-dibenzyloxy-3-pyridyl)-2-fluoro-phenyl]piperazin-1-yl]methyl]-4-fluoro-cyclohexyl]carbamate (150 mg, 214 μmol) in THF (15 mL) was added Pd/C (1.50 g, 1.41 mmol, 10 wt %) under N₂. The suspension was degassed under vacuum and purged with H₂ (432 μg, 214 mol) several times. The mixture was then stirred under H₂ (432 μg, 214 μmol) (15 psi) at 25° C. for 1 h. On completion, the reaction was filtered and the filtrate was concentrated in vacuo to give the title compound (110 mg, 98% yield) as yellow solid. LC-MS (ESI⁺) m/z 521.2 (M+H)⁺.

### Step 3—3-[4-[4-[(4-Amino-1-fluoro-cyclohexyl)methyl]piperazin-1-yl]-3-fluoro-phenyl]piperidine-2,6-dione

A solution of tert-butyl N-[4-[[4-[4-(2,6-dioxo-3-piperidyl)-2-fluoro-phenyl]piperazin-1-yl]methyl]-4-fluoro-cyclohexyl]carbamate (110 mg, 211 μmol) in DCM (0.9 mL) and TFA (0.3 mL) was stirred at 25° C. for 0.2 hr. On completion, the reaction was concentrated in vacuo to give the title compound (110 mg, 97% yield, TFA) as yellow oil. LC-MS (ESI⁺) m/z 421.2 (M+H)⁺.

### Synthesis of 1-[3-Chloro-4-[(2,6-dioxo-3-piperidyl)amino]phenyl]piperidine-4-carbaldehyde (Intermediate SQ)

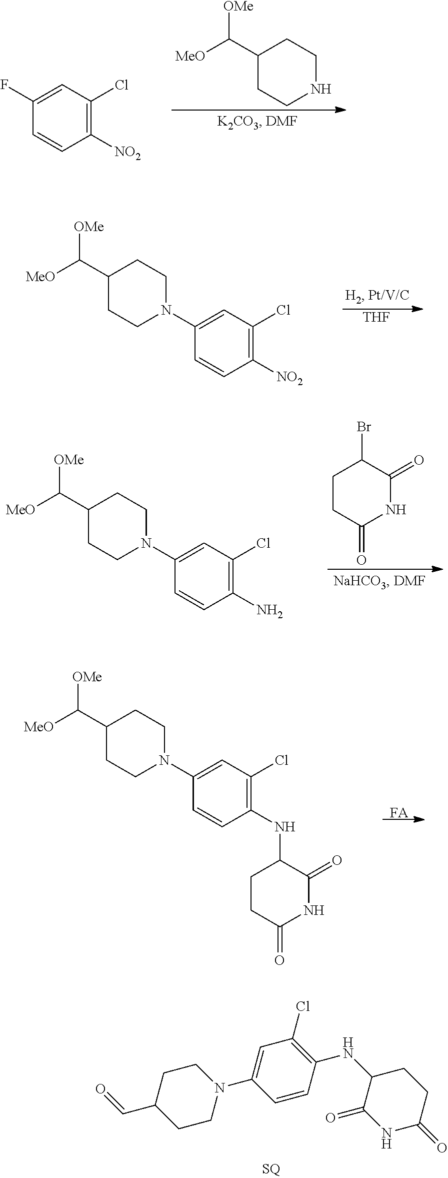

### Step 1—1-(3-Chloro-4-nitro-phenyl)-4-(dimethoxymethyl)piperidine

To a solution of 2-chloro-4-fluoro-1-nitro-benzene (4.10 g, 23.3 mmol, CAS #2106-50-5) and 4-(dimethoxymethyl)piperidine (4.09 g, 25.6 mmol, CAS #188646-83-5) in DMF (60 mL) was added K₂CO₃ (6.46 g, 46.7 mmol), then the mixture was stirred at 80° C. for 2 hrs. On completion, the mixture was filtered, diluted with water (60 mL) and extracted with EA (50 mL×3). The combined organic layer was dried over anhydrous Na₂SO₄, filtered and concentrated in vacuo to give the residue. The residue was purified by column chromatography (SiO₂, PE:EA=5:1 to 3:1) give the title compound (7.20 g, 97% yield) as yellow oil. ¹H NMR (400 MHz, DMSO-d₆) δ 8.00 (d, J=9.6 Hz, 1H), 7.06 (d, J=2.8 Hz, 1H), 6.96 (dd, J=2.4, 9.6 Hz, 1H), 4.04-4.01 (m, 1H), 3.26 (s, 6H), 3.00-2.90 (m, 3H), 1.93-1.84 (m, 1H), 1.74-1.63 (m, 2H), 1.31-1.19 (m, 2H). LC-MS (ESI⁺) m/z 314.9 (M+H)⁺.

### Step 2—2-Chloro-4-[4-(dimethoxymethyl)-1-piperidyl]aniline

To a solution of 1-(3-chloro-4-nitro-phenyl)-4-(dimethoxymethyl)piperidine (1.00 g, 3.18 mmol) in THF (20 mL) was added Pt/V/C (450 mg, 1.72 mmol) under N₂ atmosphere. The suspension was degassed and purged with H₂ 3 times. The mixture was then stirred under H₂ (15 Psi) at 25° C. for 12 hrs. On completion, the mixture was filtered and concentrated in vacuo to give the title compound (750 mg, 82% yield) as black brown oil. ¹H NMR (400 MHz, DMSO-d₆) δ 6.76 (s, 1H), 6.73-6.68 (m, 2H), 4.76 (s, 2H), 4.07 (d, J=6.8 Hz, 1H), 3.38 (d, J=11.6 Hz, 2H), 3.26 (s, 6H), 2.43 (t, J=11.2 Hz, 2H), 1.69-1.66 (m, 2H), 1.64-1.56 (m, 1H), 1.34-1.26 (m, 2H). LC-MS (ESI⁺) m/z 284.8 (M+H)⁺.

### Step 3—3-[2-Chloro-4-[4-(dimethoxymethyl)-1-piperidyl]anilino]piperidine-2,6-dione

To a solution of 2-chloro-4-[4-(dimethoxymethyl)-1-piperidyl]aniline (700 mg, 2.46 mmol) and 3-bromopiperidine-2,6-dione (707 mg, 3.69 mmol, CAS #62595-74-8) in DMF (10 mL) was added NaHCO₃ (619 mg, 7.37 mmol), then the mixture was stirred at 70° C. for 12 hrs. On completion, the mixture was filtered, diluted with water (20 mL) and extracted with EA (20 mL). The combined organic layer was dried over anhydrous Na₂SO₄, filtered and concentrated in vacuo to give the residue. The residue was purified by column chromatography (SiO₂, PE:EA=3:1 to 1:5) to give the title compound (500 mg, 51% yield) as black green oil. ¹H NMR (400 MHz, DMSO-d₆) δ 10.89 (s, 1H), 7.95 (s, 1H), 6.90 (d, J=2.8 Hz, 1H), 6.82-6.72 (m, 2H), 5.08 (d, J=6.8 Hz, 1H), 4.37-4.28 (m, 1H), 4.08 (m, 1H), 3.45 (d, J=10.4 Hz, 2H), 3.31 (d, J=4.4 Hz, 1H), 3.28-3.24 (m, 6H), 2.85-2.77 (m, 1H), 2.60-2.51 (m, 2H), 2.18-2.08 (m, 1H), 2.03-1.88 (m, 1H), 1.73-1.59 (m, 3H), 1.38-1.25 (m, 2H). LC-MS (ESI⁺) m/z 396.0 (M+H)⁺.

### Step 4—1-[3-Chloro-4-[(2,6-dioxo-3-piperidyl)amino]phenyl]piperidine-4-carbaldehyde

A solution of 3-[2-chloro-4-[4-(dimethoxymethyl)-1-piperidyl]anilino]piperidine-2,6-dione (500 mg, 1.26 mmol) in HCOOH (60.6 mg, 1.26 mmol) was stirred at 80° C. for 0.5 hr. On completion, the mixture was concentrated in vacuo to give the title compound (430 mg, 97% yield) as black green oil. LC-MS (ESI⁺) m/z 367.9 (M+18+H)⁺.

### Synthesis of 3-[4-[4-[[4-(3-Aminocyclobutyl)piperazin-1-yl]methyl]-1-piperidyl]-2-chloro-anilino]piperidine-2,6-dione (Intermediate SR)

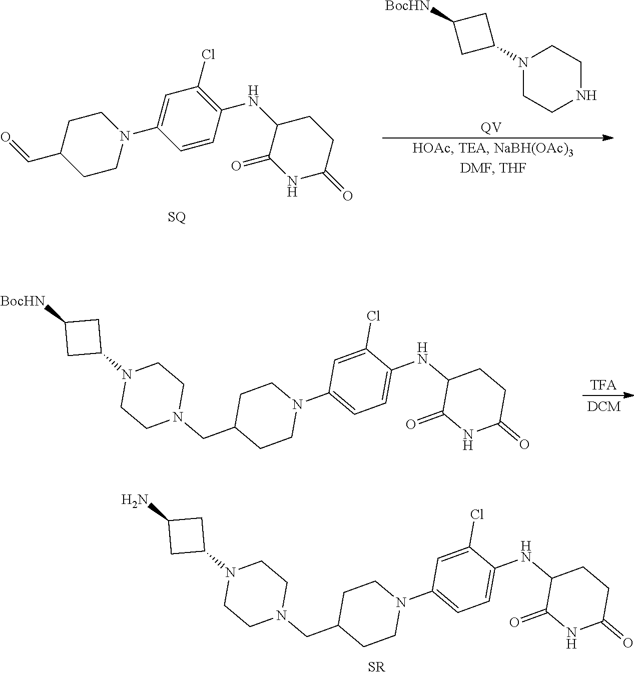

### Step 1—Tert-butyl N-[3-[4-[[1-[3-chloro-4-[(2,6-dioxo-3-piperidyl)amino]phenyl]-4-piperidyl]methyl]piperazin-1-yl]cyclobutyl]carbamate

To a solution of tert-butyl N-(3-piperazin-1-ylcyclobutyl)carbamate (80.3 mg, 314 μmol, Intermediate QV) in THF (0.5 mL) and DMF (0.5 mL) was added TEA (28.9 mg, 285 mol) until the pH=8, then 1-[3-chloro-4-[(2,6-dioxo-3-piperidyl)amino]phenyl]piperidine-4-carbaldehyde (100 mg, 285 mol, Intermediate SQ) was added, and HOAc (17.1 mg, 285 mol) was added until the pH=6. Then the mixture was stirred at 0° C. for 10 mins. Next, NaBH(OAc)₃ (90.8 mg, 428 mol) was added and the mixture was stirred at 0° C. for 0.5 hr under N₂ atmosphere. On completion, the mixture was quenched with water (1 mL) at 0° C., filtered, and the filtrate was concentrated in vacuo to give the residue. The residue was purified by prep-HPLC (column: Welch Ultimate C18 150*25 mm*5 um; mobile phase: [water (FA)-ACN]; gradient: 0%-30% B over 10 min) to give the title compound (80.0 mg, 47% yield) as white solid. ¹H NMR (400 MHz, DMSO-d6) δ 10.89 (s, 1H), 8.18 (s, 1H), 7.17 (d, J=6.8 Hz, 1H), 6.89 (d, J=2.4 Hz, 1H), 6.82-6.77 (m, 1H), 6.76-6.72 (m, 1H), 5.07 (d, J=6.4 Hz, 1H), 4.36-4.26 (m, 1H), 3.86-3.85 (m, 1H), 3.42 (d, J=11.6 Hz, 2H), 2.85-2.72 (m, 2H), 2.57-2.56 (m, 1H), 2.52 (s, 2H), 2.47 (s, 2H), 2.44-2.24 (m, 6H), 2.18-2.16 (m, 2H), 2.14-2.06 (m, 3H), 2.02-1.88 (m, 3H), 1.75-1.72 (m, 2H), 1.62-1.51 (m, 1H), 1.37 (s, 9H), 1.25-1.12 (m, 2H). LC-MS (ESI⁺) m/z 589.3 (M+H)⁺.

### Step 2—3-[4-[4-[[4-(3-Aminocyclobutyl)piperazin-1-yl]methyl]-1-piperidyl]-2-chloro-anilino] piperidine-2,6-dione

To a solution of tert-butyl N-[3-[4-[[1-[3-chloro-4-[(2,6-dioxo-3-piperidyl)amino]phenyl]-4-piperidyl] methyl]piperazin-1-yl]cyclobutyl]carbamate (70.0 mg, 118 μmol) in DCM (1 mL) was added TFA (13.5 mg, 118 mol, 8.83 μL), then the mixture was stirred at 25° C. for 0.5 hr. On completion, the mixture was concentrated in vacuo to give the title compound (60.0 mg, 83% yield, TFA) as black brown oil. LC-MS (ESI⁺) m/z 489.2 (M+H)⁺.

### Synthesis of 3-[4-[4-[(4-Aminocyclohexyl)methyl]piperazin-1-yl]-5-fluoro-2-methoxy-anilino]piperidine-2,6-dione (Intermediate SS)

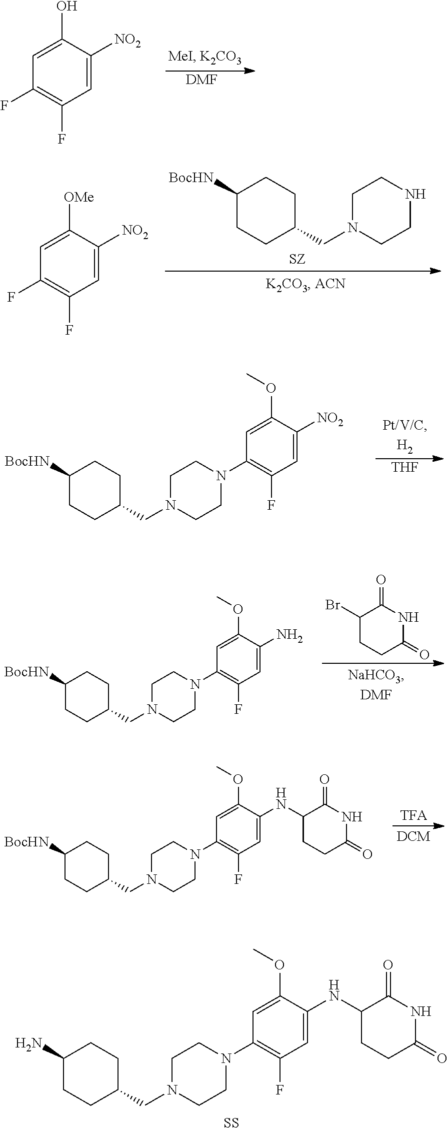

### Step 1—1,2-Difluoro-4-methoxy-5-nitro-benzene

To a solution of 4,5-difluoro-2-nitro-phenol (3 g, 17.13 mmol, CAS #55346-97-9) in DMF (20 mL) was added K₂CO₃ (7.10 g, 51.4 mmol) and MeI (3.65 g, 25.7 mmol, 1.60 mL). The reaction was stirred at 25° C. or 4 hrs. On completion, the reaction was diluted with EA (50 mL). The organic layer was washed with water (50 mL×3), dried over Na₂SO₄, filtered and concentrated in vacuo to give the title compound (2.92 g, 90% yield) as yellow solid. ¹H NMR (400 MHz, DMSO-d₆) δ 8.23 (dd, J=8.4, 10.0 Hz, 1H), 7.63 (dd, J=6.8, 12.4 Hz, 1H), 3.93 (s, 3H).

### Step 2—Tert-butyl N-[4-[[4-(2-fluoro-5-methoxy-4-nitro-phenyl)piperazin-1-yl]methyl]cyclohexyl]carbamate

To a solution of tert-butyl N-[4-(piperazin-1-ylmethyl)cyclohexyl]carbamate (1.56 g, 5.23 mmol, Intermediate SZ) and 1,2-difluoro-4-methoxy-5-nitro-benzene (900 mg, 4.76 mmol) in DMF (25 mL) was added K₂CO₃ (1.97 g, 14.2 mmol). The reaction was then stirred at 25° C. for 16 hrs. On completion, the reaction was diluted with EA (80 mL). The organic layer was washed with water (50 mL×3), dried over Na₂SO₄ and concentrated in vacuo. The residue was purified by column chromatography (SiO₂, DCM/EA=1/0 to 1/1) to give the title compound ((1.71 g, 77% yield) as yellow oil. ¹H NMR (400 MHz, DMSO-d₆) δ 7.84 (d, J=13.2 Hz, 1H), 6.71 (d, J=7.6 Hz, 1H), 6.66 (d, J=7.6 Hz, 1H), 3.93 (s, 3H), 3.31 (s, 5H), 3.29 (d, J=4.4 Hz, 4H), 2.12 (d, J=7.2 Hz, 2H), 1.77 (d, J=11.2 Hz, 4H), 1.37 (s, 10H), 1.15-1.06 (m, 2H), 0.93-0.82 (m, 2H); LC-MS (ESI⁺) m/z 467.2 (M+H)⁺.

### Step 3—Tert-butyl N-[4-[[4-(4-amino-2-fluoro-5-methoxy-phenyl)piperazin-1-yl]methyl]cyclohexyl]carbamate

To a solution of tert-butyl N-[4-[[4-(2-fluoro-5-methoxy-4-nitro-phenyl)piperazin-1-yl]methyl]cyclohexyl]carbamate (1.6 g, 3.43 mmol) in THF (10 mL) was added Pt/V/C (1.60 g, 183 μmol, 3 wt %) under Ar. The suspension was degassed under vacuum and purged with H₂ several times. The mixture was stirred under H₂ (15 psi) at 25° C. for 16 hrs. On completion, the reaction mixture was filtered and the filtrate was concentrated in vacuo to give the title compound (1.44 g, 96% yield) as white solid. ¹H NMR (400 MHz, DMSO-d₆) δ 6.68 (d, J=7.6 Hz, 1H), 6.53 (d, J=8.0 Hz, 1H), 6.41 (d, J=13.6 Hz, 1H), 4.58 (s, 2H), 3.72 (s, 3H), 3.22-3.11 (m, 1H), 2.86 (s, 4H), 2.43 (s, 4H), 2.10 (d, J=7.2 Hz, 2H), 1.76 (d, J=11.2 Hz, 4H), 1.38-1.36 (m, 9H), 1.35 (s, 1H), 1.17-1.06 (m, 2H), 0.92-0.79 (m, 2H); LC-MS (ESI⁺) m/z 437.3 (M+H)⁺.

### Step 4—Tert-butyl N-[4-[[4-[4-[(2,6-dioxo-3-piperidyl)amino]-2-fluoro-5-methoxy-phenyl] piperazin-1-yl]methyl]cyclohexyl]carbamate

To a solution of tert-butyl N-[4-[[4-(4-amino-2-fluoro-5-methoxy-phenyl)piperazin-1-yl]methyl]cyclohexyl]carbamate (500 mg, 1.15 mmol) and 3-bromopiperidine-2,6-dione (329 mg, 1.72 mmol, CAS#62595-74-8) in DMF (8 mL) was added NaHCO₃ (288 mg, 3.44 mmol, 133 μL). The reaction was stirred at 80° C. for 6 hrs. On completion, the reaction was diluted with EA (30 mL). The organic layer was washed with water (30 mL×3), dried over Na₂SO₄ and concentrated in vacuo. The residue was purified by prep-HPLC (column: Phenomenex Luna C18 150*25 mm*10 um; mobile phase: [water (FA)-ACN]; gradient: 11%-41% B over 10 min) to give the title compound (340 mg, 54% yield) as brown solid. ¹H NMR (400 MHz, DMSO-d₆) δ 10.86 (s, 1H), 6.71 (d, J=8.4 Hz, 1H), 6.60 (d, J=8.0 Hz, 1H), 6.54 (d, J=14.0 Hz, 1H), 5.08 (d, J=6.8 Hz, 1H), 4.28-4.20 (m, 1H), 3.78 (s, 3H), 3.33 (s, 4H), 3.20-3.09 (m, 1H), 2.89 (s, 4H), 2.83-2.74 (m, 1H), 2.55 (d, J=3.2 Hz, 2H), 2.15-2.08 (m, 3H), 1.98-1.86 (m, 1H), 1.76 (d, J=10.4 Hz, 4H), 1.38-1.34 (m, 9H), 1.18-1.04 (m, 2H), 0.86 (q, J=11.6 Hz, 2H); LC-MS (ESI⁺) m/z 548.2 (M+H)⁺.

### Step 5—3-[4-[4-[(4-Aminocyclohexyl)methyl]piperazin-1-yl]-5-fluoro-2-methoxy-anilino]piperidine-2,6-dione

To a solution of tert-butyl N-[4-[[4-[4-[(2,6-dioxo-3-piperidyl)amino]-2-fluoro-5-methoxy-phenyl]piperazin-1-yl]methyl]cyclohexyl]carbamate (120 mg, 219 μmol) in DCM (1.5 mL) was added TFA (460 mg, 4.04 mmol, 0.3 mL). The reaction was then stirred at 25° C. for 1 hr. On completion, the reaction was concentrated in vacuo to give the title compound (123 mg, 99% yield, TFA) as brown oil. LC-MS (ESI⁺) m/z 448.1 (M+H)⁺.

### 4-Bromo-N-[4-[[tert-butyl(dimethyl)silyl]oxymethyl]cyclohexyl]-3-fluoro-benzenesulfonamide (Intermediate ST)

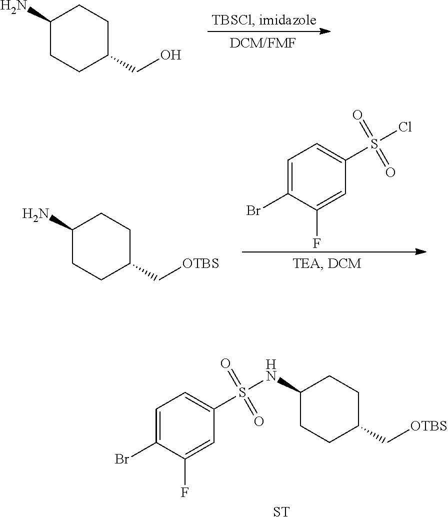 [SMILES: N[C@H]1CC[C@H](CO)CC1 ; CC(C)(C)[Si](C)(C)OC[C@H]1CC[C@H](N)CC1 ; O=S(=O)(Cl)c1ccc(Br)c(F)c1 ; CC(C)(C)[Si](C)(C)OC[C@H]1CC[C@H](NS(=O)(=O)c2ccc(Br)c(F)c2)CC1 ; "ST"]

### Step 1—4-[[Tert-butyl(dimethyl)silyl]oxymethyl]cyclohexanamine

To a solution of (4-aminocyclohexyl)methanol (500 mg, 3.87 mmol, CAS #1467-84-1) in DCM (7 mL) and DMF (1 mL) was added imidazole (526 mg, 7.74 mmol), then TBSCl (641 mg, 4.26 mmol) was added at 0° C. and the mixture was stirred at 25° C. for 12 hrs. On completion, the mixture was diluted with water (10 mL) and extracted with DCM (10 mL×3). The combined organic layer was dried over anhydrous Na₂SO₄, filtered and concentrated in vacuo to give the title compound (880 mg, 93% yield) as yellow oil. ¹H NMR (400 MHz, ClCD₃) δ 3.37 (d, J=6.4 Hz, 2H), 2.62-2.58 (m, 1H), 1.91-1.85 (m, 2H), 1.78-1.73 (m, 2H), 1.43-1.33 (m, 1H), 1.14-1.05 (m, 3H), 0.952-9.14 (m, 3H), 0.86 (s, 9H), 0.01 (s, 6H).

### Step 2—4-Bromo-N-[4-[[tert-butyl(dimethyl)silyl]oxymethyl]cyclohexyl]-3-fluoro-benzenesulfonamide

To a solution of 4-[[tert-butyl(dimethyl)silyl]oxymethyl]cyclohexanamine (880 mg, 3.61 mmol) in DCM (6 mL) was added TEA (831 mg, 8.21 mmol), then 4-bromo-3-fluoro-benzenesulfonyl chloride (898 mg, 3.29 mmol, CAS #351003-51-5) was added and the mixture was stirred at 25° C. for 1 hr. On completion, the mixture was diluted with water (10 mL) and extracted with DCM (10 mL×3). The combined organic layer was dried over anhydrous Na₂SO₄, filtered and concentrated in vacuo to give the residue. The residue was purified by column chromatography (SiO₂, PE:EA=10:1 to 1:1) to give the title compound (800 mg, 50% yield) as yellow solid. ¹H NMR (400 MHz, DMSO-d₆) δ 7.96 (dd, J=6.8, 8.4 Hz, 1H), 7.87 (d, J=7.6 Hz, 1H), 7.73 (dd, J=1.6, 8.4 Hz, 1H), 7.58 (dd, J=1.6, 8.4 Hz, 1H), 3.30 (s, 1H), 2.97-2.86 (m, 1H), 1.68-1.57 (m, 5H), 1.29-1.20 (m, 1H), 1.15-1.06 (m, 2H), 0.88 (s, 2H), 0.83 (s, 9H), -0.02 (s, 6H).

### Synthesis of 3-Fluoro-N-(4-formylcyclohexyl)-4-[[4-(1-tetrahydropyran-4-ylpyrazol-4-yl)-5-(trifluoro methyl)pyrimidin-2-yl]amino]benzenesulfonamide (Intermediate SU)

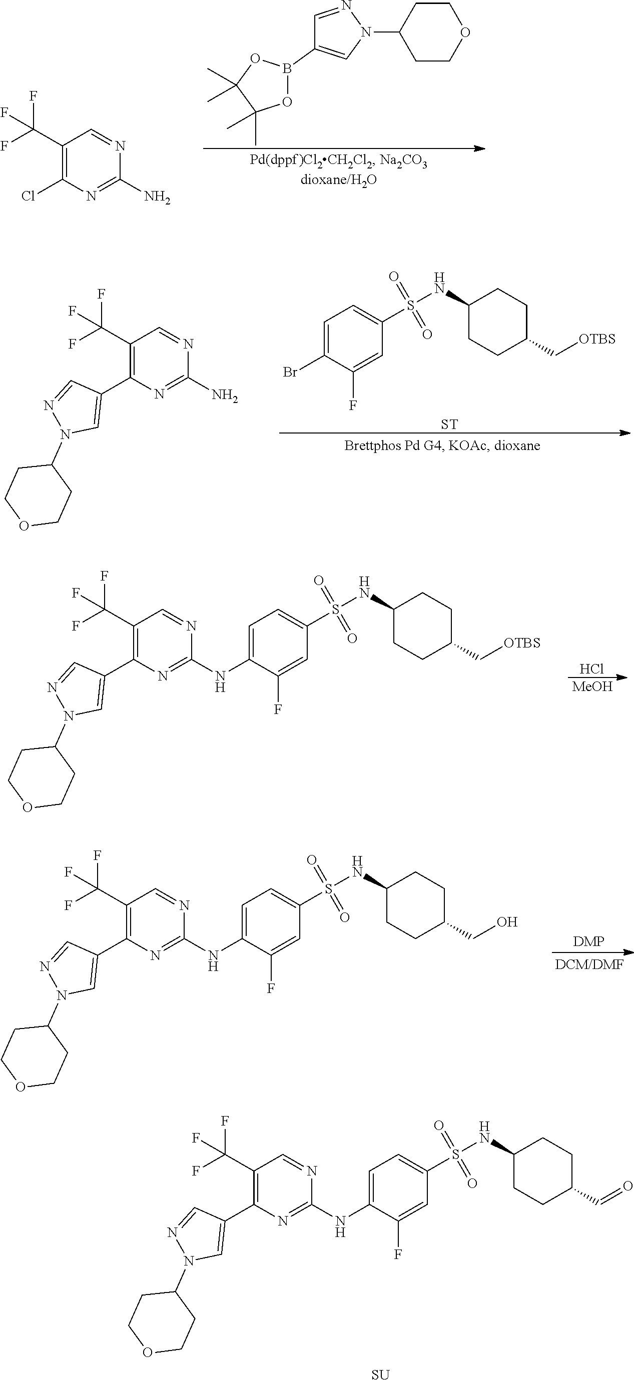

### Step 1—4-(1-Tetrahydropyran-4-ylpyrazol-4-yl)-5-(trifluoromethyl)pyrimidin-2-amine

To a solution of 4-chloro-5-(trifluoromethyl)pyrimidin-2-amine (1.00 g, 5.06 mmol, CAS#1201657-24-0) and 1-tetrahydropyran-4-yl-4-(4,4,5,5-tetramethyl-1,3,2-dioxaborolan-2-yl)pyrazole (938 mg, 3.37 mmol, CAS #1040377-03-4) in dioxane (10 mL) and H₂O (2 mL) was added Pd(dppf)Cl₂·CH₂Cl₂ (275 mg, 337 mol) and Na₂CO₃ (715 mg, 6.75 mmol). Then the reaction was stirred at 90° C. for 6 hrs under N₂ atmosphere. On completion, the mixture was filtered, diluted with water (10 mL) and extracted with EA (10 mL×3). The combined organic layer was dried over anhydrous Na₂SO₄, filtered and concentrated in vacuo to give the residue. The residue was purified by column chromatography (SiO₂, PE/EA=40% to 70%) to give the title compound (660 mg, 62% yield) as yellow solid. ¹H NMR (400 MHz, DMSO-d6) δ 8.51 (s, 1H), 8.21 (s, 1H), 7.91 (s, 1H), 7.35 (s, 2H), 4.59-4.47 (m, 1H), 4.00-3.94 (m, 2H), 3.51-3.41 (m, 2H), 2.00-1.94 (m, 4H). LC-MS (ESI⁺) m/z 313.9 (M+H)⁺.

### Step 2—N-[4-[[tert-butyl(dimethyl)silyl]oxymethyl]cyclohexyl]-3-fluoro-4-[[4-(1-tetrahydropyran-4-ylpyrazol-4-yl)-5-(trifluoromethyl)pyrimidin-2-yl]amino]benzenesulfonamide

To a solution of 4-bromo-N-[4-[[tert-butyl(dimethyl)silyl]oxymethyl]cyclohexyl]-3-fluoro-benzenesulfonamide (250 mg, 520 μmol, Intermediate ST) and 4-(1-tetrahydropyran-4-ylpyrazol-4-yl)-5-(trifluoromethyl)pyrimidin-2-amine (108 mg, 346 μmol) in dioxane (7 mL) was added KOAc (68.0 mg, 693 μmol) and dicyclohexyl-[3,6-dimethoxy-2-(2,4,6-triisopropylphenyl)phenyl]phosphane methanesulfonate [2-[2-(methylamino)phenyl]phenyl]palladium(1+) (31.9 mg, 34.6 μmol). Then the mixture was stirred at 90° C. for 14 hrs under N₂ atmosphere. On completion, the mixture was filtered, diluted with water (5 mL) and extracted with EA (5 mL×3). The combined organic layer was dried over anhydrous Na₂SO₄, filtered and concentrated in vacuo to give the residue. The residue was purified by prep-HPLC (column: Welch Ultimate C18 150*25 mm*5 um; mobile phase: [water (TFA) -ACN]; gradient: 80%-100% B over 2 min) to give the title compound (58.0 mg, 23% yield) as yellow solid. LC-MS (ESI⁺) m/z 713.4 (M+H)⁺.

### Step 3—3-Fluoro-N-[4-(hydroxymethyl)cyclohexyl]-4-[[4-(1-tetrahydropyran-4-ylpyrazol-4-yl)-5-(trifluoromethyl)pyrimidin-2-yl]amino]benzenesulfonamide

To a solution of N-[4-[[tert-butyl(dimethyl)silyl]oxymethyl]cyclohexyl]-3-fluoro-4-[[4-(1-tetrahydropyran-4-ylpyrazol-4-yl)-5-(trifluoromethyl)pyrimidin-2-yl]amino]benzenesulfonamide (50.0 mg, 70.1 mol) in MeOH (1 mL) was added HCl (12 M, 0.01 mL), then the mixture was stirred at 25° C. for 0.5 hr. On completion, the mixture was concentrated in vacuo to give the title compound (38.0 mg, 90% yield) as yellow solid. ¹H NMR (400 MHz, DMSO-d₆) δ 10.08 (s, 1H), 8.79 (s, 1H), 8.29 (s, 1H), 8.07 (t, J=8.4 Hz, 1H), 7.97 (s, 1H), 7.73-7.65 (m, 3H), 4.62-4.52 (m, 1H), 4.01-3.92 (m, 2H), 3.50-3.43 (m, 2H), 3.13 (d, J=6.4 Hz, 2H), 2.95-2.85 (m, 1H), 2.02-1.95 (m, 4H), 1.69-1.60 (m, 4H), 1.26-1.08 (m, 4H), 0.86-0.76 (m, 2H). LC-MS (ESI⁺) m/z 599.1 (M+H)⁺.

### Step 4—3-Fluoro-N-(4-formylcyclohexyl)-4-[[4-(1-tetrahydropyran-4-ylpyrazol-4-yl)-5-(trifluoromethyl)pyrimidin-2-yl]amino]benzenesulfonamide

To a solution of 3-fluoro-N-[4-(hydroxymethyl)cyclohexyl]-4-[[4-(1-tetrahydropyran-4-ylpyrazol-4-yl)-5-(trifluoromethyl)pyrimidin-2-yl]amino]benzenesulfonamide (38.0 mg, 63.4 μmol) in DCM (1 mL) and DMF (1 mL) was added DMP (40.3 mg, 95.2 μmol). Then the mixture was stirred at 0° C. for 2 hrs. On completion, the mixture was quenched with saturated Na₂S₂O₃ (2 mL) and saturated NaHCO₃ (2 mL) at 0° C., diluted with water (4 mL) and extracted with DCM (3 mL×3). The combined organic layer was dried over anhydrous Na₂SO₄, filtered and concentrated in vacuo to give the title compound (35.0 mg, 92% yield) as yellow oil. LC-MS (ESI⁺) m/z 597.2 (M+H)⁺.

### Synthesis of 3-(4-(4-((4-Aminobicyclo[2.2.1]heptan-1-yl)methyl)piperazin-1-yl)-2-fluorophenyl) piperidine-2,6-dione (Intermediate SV)

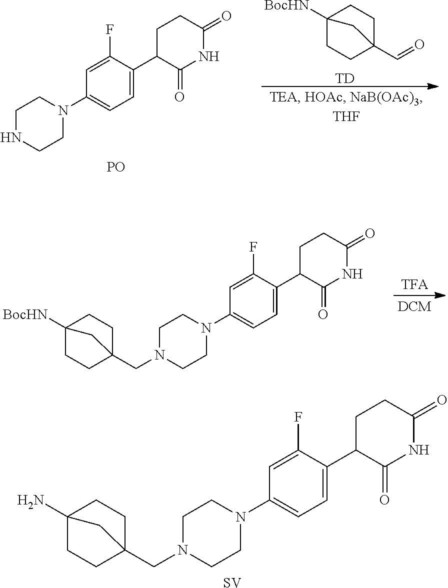

### Step 1—Tert-butyl (4-((4-(4-(2,6-dioxopiperidin-3-yl)-3-fluorophenyl)piperazin-1-yl) methyl)bicyclo[2.2.1]heptan-1-yl)carbamate

To a solution of 3-(2-fluoro-4-piperazin-1-yl-phenyl)piperidine-2,6-dione (155 mg, 382 μmol, TFA, Intermediate PO) in THF (3 mL) was added TEA (38.6 mg, 382 μmol, 53.2 μL), HOAc (45.9 mg, 764 μmol, 43.7 μL) and tert-butyl N-(4-formyl norbornan-1-yl)carbamate (91.51 mg, 382.39 μmol, Intermediate TD). The mixture was stirred at 20° C. for 0.5 hr. Then, NaBH(OAc)₃ (121 mg, 573 mol) was added and the mixture was stirred at 20° C. for 0.5 hr. On completion, the reaction was diluted with H₂O (20 mL) and extracted with EtOAc (60 mL). The organic layer was washed with brine (20 mL), dried over Na₂SO₄, filtered and concentrated in vacuo. The residue was purified by prep-HPLC (column: Phenomenex luna C18 150*25 mm*10 um; mobile phase: [water (FA)-ACN]; gradient: 10%-40% B over 10 min) to give the title compound (120 mg, 60% yield) as white solid. ¹H NMR (400 MHz, DMSO-d₆) δ 10.80 (s, 1H), 7.17-7.03 (m, 1H), 7.00-6.85 (m, 1H), 6.81-6.67 (m, 2H), 3.89 (dd, J=4.8, 12.4 Hz, 1H), 3.32 (s, 10H), 3.20-3.03 (m, 3H), 2.81-2.64 (m, 2H), 2.21-2.08 (m, 1H), 2.00-1.90 (m, 1H), 1.87-1.73 (m, 2H), 1.68-1.49 (m, 5H), 1.37 (s, 9H).

### Step 2—3-(4-(4-((4-Aminobicyclo[2.2.1]heptan-1-yl)methyl)piperazin-1-yl)-2-fluorophenyl) piperidine-2,6-dione

A solution of tert-butyl N-[4-[[4-[4-(2,6-dioxo-3-piperidyl)-3-fluoro-phenyl] piperazin-1-yl]methyl]norbornan-1-yl]carbamate (60 mg, 116 μmo) in CH₂Cl₂ (1 mL) and HCl/dioxane (4 M, 2 mL) was stirred at 20° C. for 1.5 hrs. On completion, the reaction was concentrated in vacuo to give the title compound (52 mg, 98% yield) as white solid. LC-MS (ESI⁺) m/z 415.2 (M+H)⁺.

### Synthesis of 3-[4-[4-[(4-aminocyclohexyl)methyl]piperazin-1-yl]-3-fluoro-anilino]piperidine-2,6-dione (Intermediate SW)

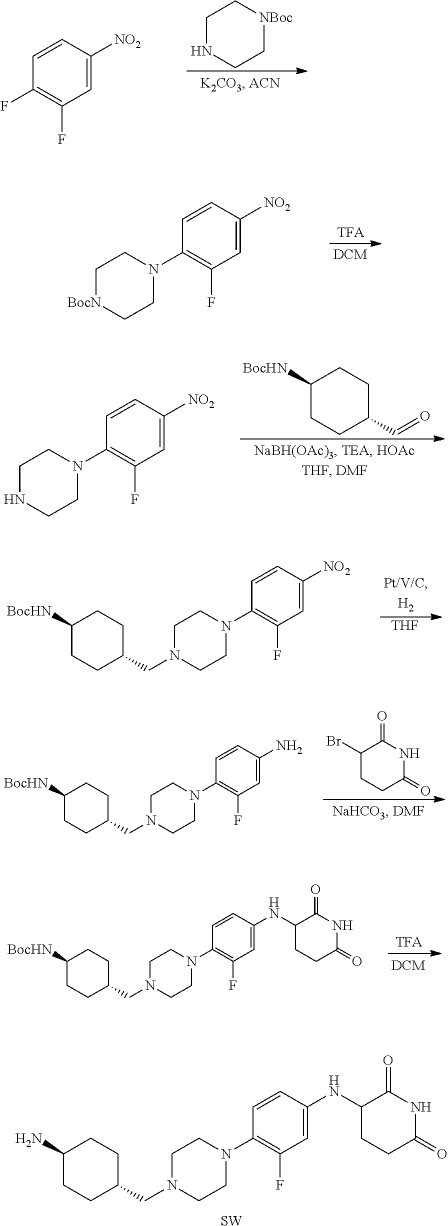

### Step 1—Tert-butyl 4-(2-fluoro-4-nitro-phenyl) piperazine-1-carboxylate

To a solution of 1,2-difluoro-4-nitro-benzene (5.91 mmol, 654 uL, CAS #369-34-6) and tert-butyl piperazine-1-carboxylate (1.00 g, 5.37 mmol, CAS #143238-38-4) in DMF (10 mL) was added K₂CO₃ (2.23 g, 16.1 mmol), then the mixture was stirred at 90° C. for 12 hrs. On completion, the mixture was diluted with EA (20 mL) and H₂O (40 mL), then extracted with EA (4×20 mL). The combined organic layers were washed with NaCl (60 mL), dried over sodium sulfate, filtered and concentrated in vacuo to give the residue. The residue was purified by column chromatography (SiO₂, PE:EA=11:1 to 7:1) to give the title compound (1.50 g, 86% yield) as a yellow solid. ¹H NMR (400 MHz, DMSO-d₆) δ 8.14-7.93 (m, 2H), 7.18 (t, J=9.2 Hz, 1H), 3.48 (s, 4H), 3.29-3.21 (m, 4H), 1.42 (s, 9H). LC-MS (ESI⁺) m/z 270.1 (M−56)+.

### Step 2—1-(2-Fluoro-4-nitro-phenyl)piperazine

To a solution of tert-butyl 4-(2-fluoro-4-nitro-phenyl) piperazine-1-carboxylate (1.50 g, 4.61 mmol) in DCM (6 mL) was added TFA (26.9 mmol, 2 mL), then the mixture was stirred at 25° C. for 1 hr. On completion, the mixture concentrated in vacuo to give the title compound (1.50 g, 96% yield, TFA) as a yellow oil. LC-MS (ESI⁺) m/z 226.0 (M+H)⁺.

### Step 3—Tert-butyl N-[4-[[4-(2-fluoro-4-nitro-phenyl) piperazin-1-yl] methyl]cyclohexyl]carbamate

To a solution of 1-(2-fluoro-4-nitro-phenyl) piperazine (1.50 g, 6.66 mmol, TFA) in THF (10 mL) and DMF (0.5 mL) was added TEA (6.66 mmol, 927 μL), then tert-butyl N-(4-formylcyclohexyl) carbamate (1.82 g, 7.99 mmol, CAS #181308-56-5) and AcOH (6.66 mmol, 381 L) was added. The mixture was then stirred at 25° C. for 0.2 hr. Next, NaBH(OAc)₃ (2.82 g, 13.3 mmol) was added and the mixture was stirred at 25° C. for 2 hrs. On completion, the mixture concentrated in vacuo to give the residue. The residue was purified by prep-HPLC (column: YMC Triart C18 70*250 mm*7 um; mobile phase: [water (FA)-ACN]; gradient: 20%-50% B over 20 min) to give the title compound (2.00 g, 69% yield) as a yellow solid. ¹H NMR (400 MHz, DMSO-d₆) δ 8.06-7.99 (m, 2H), 7.19 (t, J=8.8 Hz, 1H), 6.78-6.67 (m, 1H), 3.33 (s, 8H), 3.19-3.13 (m, 1H), 2.72-2.62 (m, 1H), 2.39-2.26 (m, 1H), 1.77 (d, J=10.8 Hz, 4H), 1.55-1.44 (m, 1H), 1.37 (s, 9H), 1.21-1.09 (m, 2H), 0.92 (d, J=8.0 Hz, 2H). LC-MS (ESI⁺) m/z 437.1 (M+H)⁺.

### Step 4—Tert-butyl N-[4-[[4-(4-amino-2-fluoro-phenyl)piperazin-1-yl]methyl]cyclohexyl]carbamate

To a solution of tert-butyl N-[4-[[4-(2-fluoro-4-nitro-phenyl)piperazin-1-yl]methyl]cyclohexyl]carbamate (1.00 g, 2.29 mmol) in THF (15 mL) was added Pt/V/C (800 mg, 3.06 mmol), then the mixture was purged with H₂ for three times. The mixture was stirred at 25° C. under 15 PSI H₂ for 3 hrs. On completion, the mixture was filtered and concentrated in vacuo to give the title compound (900 mg, 96% yield) as a brown solid. ¹H NMR (400 MHz, DMSO-d₆) δ 6.83-6.60 (m, 2H), 6.39-6.23 (m, 2H), 5.20-4.77 (m, 2H), 3.23-3.03 (m, 3H), 2.84 (d, J=2.0 Hz, 4H), 2.24-1.95 (m, 3H), 1.76 (d, J=10.0 Hz, 4H), 1.39-1.34 (m, 9H), 1.25-1.03 (m, 4H), 0.98-0.83 (m, 2H). LC-MS (ESI⁺) m/z 407.2 (M+H)⁺.

### Step 5—Tert-butyl N-[4-[[4-[4-[(2,6-dioxo-3-piperidyl)amino]-2-fluoro-phenyl]piperazin-1-yl]methyl]cyclohexyl]carbamate

To a solution of tert-butyl N-[4-[[4-(4-amino-2-fluoro-phenyl) piperazin-1-yl]methyl]cyclohexyl]carbamate (900 mg, 2.21 mmol) in DMF (15 mL) was added NaHCO₃ (557 mg, 6.64 mmol). Then 3-bromopiperidine-2,6-dione (1.28 g, 6.64 mmol, CAS #62595-74-8) was added and the mixture was stirred at 80° C. for 12 hrs. On completion, the mixture was concentrated in vacuo to give the residue. The residue was purified by prep-HPLC (column: Phenomenex luna C18 (250*70 mm, 10 um); mobile phase: [water(FA)-ACN]; gradient:15%-45% B over 15 min), title compound (500 mg, 44% yield) as a violet solid. ¹H NMR (400 MHz, DMSO-d₆) δ 10.82 (d, J=8.0 Hz, 1H), 6.82 (t, J=9.2 Hz, 1H), 6.70 (d, J=8.0 Hz, 1H), 6.55-6.47 (m, 1H), 6.45-6.39 (m, 1H), 5.82 (d, J=7.2 Hz, 1H), 4.33-4.15 (m, 1H), 3.18-3.14 (m, 2H), 2.88 (s, 4H), 2.78-2.67 (m, 2H), 2.60-2.57 (m, 1H), 2.35-2.31 (m, 1H), 2.28-2.16 (m, 3H), 2.13-2.03 (m, 2H), 1.91-1.81 (m, 1H), 1.76 (d, J=11.2 Hz, 4H), 1.37 (s, 9H), 1.17-1.08 (m, 2H), 0.94-0.82 (m, 2H). LC-MS (ESI⁺) m/z 518.2 (M+H)⁺.

### Step 6—3-[4-[4-[(4-Aminocyclohexyl)methyl]piperazin-1-yl]-3-fluoro-anilino]piperidine-2,6-dione

To a solution of tert-butyl N-[4-[[4-[4-[(2,6-dioxo-3-piperidyl)amino]-2-fluoro-phenyl]piperazin-1-yl] methyl]cyclohexyl]carbamate (136 mg, 262 μmol) in DCM (1 mL) was added TFA (46 mg, 4.04 mmol, 0.3 mL). The reaction was then stirred at 25° C. for 1 hr. On completion, the reaction was concentrated in vacuo to give the title compound (139 mg, 99% yield, TFA) as yellow oil. LC-MS (ESI⁺) m/z 418.0 (M+H)⁺.

### Synthesis of (1s,4s)-4-(2-((4-(benzylthio)-2-methylphenyl)amino)-5-(trifluoromethyl)pyrimidin-4-yl)-1-methylcyclohexan-1-ol (Intermediate SX) and (1r,4r)-4-(2-((4-(benzylthio)-2-methylphenyl)amino)-5-(trifluoromethyl)pyrimidin-4-yl)-1-methylcyclohexan-1-ol (Intermediate SY)

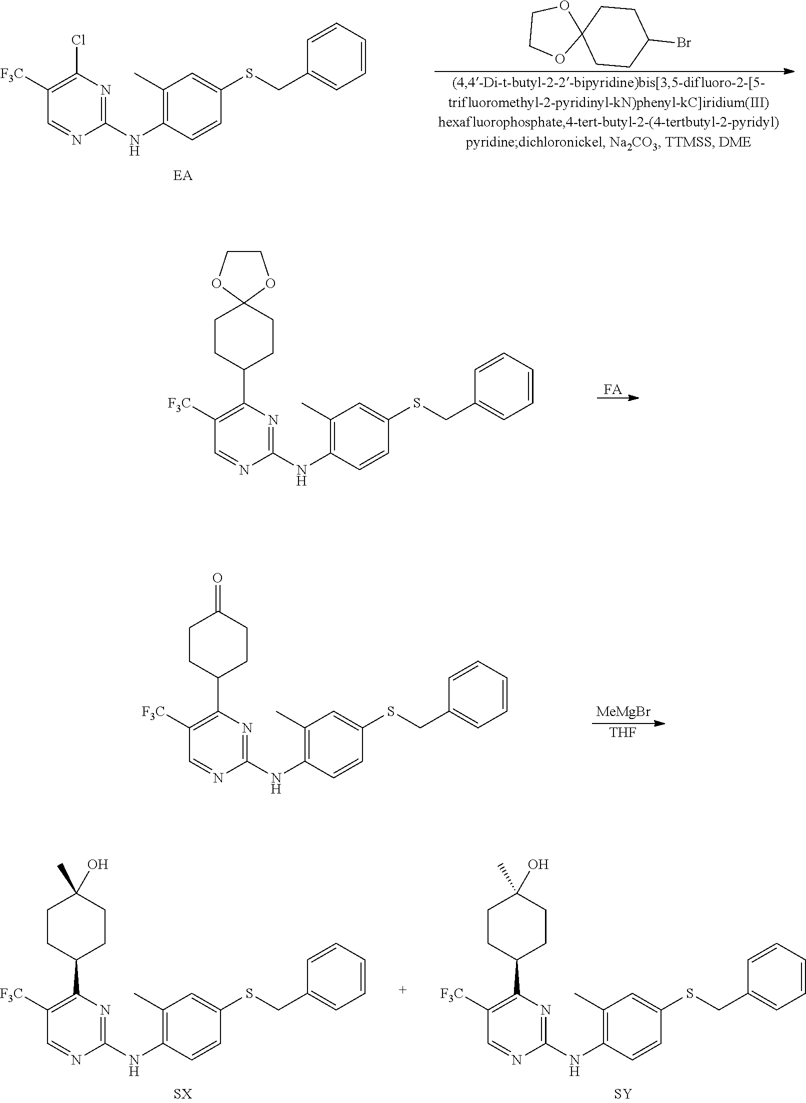

### Step 1—N-(4-benzylsulfanyl-2-methyl-phenyl)-4-(1,4-dioxaspiro[4.5]decan-8-yl)-5-(trifluoromethyl)pyrimidin-2-amine

To a solution of N-(4-benzylsulfanyl-2-methyl-phenyl)-4-chloro-5-(trifluoromethyl)pyrimidin-2-amine (1.20 g, 2.93 mmol, Intermediate EA) and 8-bromo-1,4-dioxaspiro[4.5]decane (647 mg, 2.93 mmol, CAS #68278-51-3) in DME (12 mL) was added Na₂CO₃ (620 mg, 5.86 mmol,), TTMSS (728 mg, 2.93 mmol), (4,4′-Di-t-butyl-2,2′-bipyridine)bis[3,5-difluoro-2-[5-trifluoromethyl-2-pyridinyl-kN)phenyl-kC]iridium(III) hexafluorophosphate (32.8 mg, 29.2 umol, CAS #870987-63-6) and 4-tert-butyl-2-(4-tertbutyl-2-pyridyl) pyridine dichloronickel (11.6 mg, 29.2 umol). The mixture was then stirred at 25° C. for 14 hrs. On completion, the reaction mixture was filtered and concentrated in vacuo. The residue was diluted with water (60 mL) and extracted with EA (40 mL×2). The combined organic layer was dried over Na₂SO₄, filtered and concentrated in vacuo. The residue was purified by column chromatography (SiO₂, PE:EA=50:1 to PE:EA=20:1, PE:EA=5:1, PL:Rf=0.3) to give the title compound (900 mg, 60% yield) as white solid. ¹H NMR (400 MHz, DMSO-d₆) δ 9.44 (s, 1H), 8.52 (s, 1H), 7.38-7.33 (m, 2H), 7.33-7.27 (m, 3H), 7.26-7.20 (m, 2H), 7.15-7.12 (m, 1H), 4.22 (s, 2H), 3.86 (s, 4H), 2.79 (t, J=11.4 Hz, 1H), 2.16 (s, 3H), 1.96-1.84 (m, 2H), 1.80-1.64 (m, 4H), 1.59-1.51 (m, 2H).

### Step 2—4-[2-(4-Benzylsulfanyl-2-methyl-anilino)-5-(trifluoromethyl)pyrimidin-4-yl]cyclohexanone

A solution of N-(4-benzylsulfanyl-2-methyl-phenyl)-4-(1,4-dioxaspiro[4.5]decan-8-yl)-5-(trifluoromethyl)pyrimidin-2-amine (100 mg, 193 umol) in FA (1 mL) was stirred at 25° C. for 16 hrs. On completion, the reaction mixture was concentrated in vacuo. Then, CH₃CN (1 mL) and H₂O (10 mL) was added to the residue and the solution was lyophilized to give the title compound (80.0 mg, 87% yield) as white solid. LCMS (ESI⁺) m/z 472.4 (M+H)⁺.

### Step 3—(1s,4s)-4-(2-((4-(benzylthio)-2-methylphenyl)amino)-5-(trifluoromethyl)pyrimidin-4-yl)-1-methylcyclohexan-1-ol and (1r,4r)-4-(2-((4-(benzylthio)-2-methylphenyl)amino)-5-(trifluoromethyl)pyrimidin-4-yl)-1-methylcyclohexan-1-ol

To a solution of 4-[2-(4-benzylsulfanyl-2-methyl-anilino)-5-(trifluoromethyl)pyrimidin-4-yl]cyclohexanone (60.0 mg, 127 umol) in THF (1 mL) was added MeMgBr (3 M, 212 uL) at 0° C. The mixture was stirred at 25° C. for 16 hrs. On completion, the reaction mixture was quenched with NaHCO₃ solution (20 mL). The residue was diluted with water (40 mL) and extracted with EA (20 mL×2). The combined organic layer was washed with brine (20 mL×2) and dried over Na₂SO₄, filtered and the filtrate was concentrated in vacuo. The residue was purified by column chromatography by prep-TLC (SiO₂, PE:EA=2:1) to give (1s,4s)-4-(2-((4-(benzylthio)-2-methylphenyl)amino)-5-(trifluoromethyl)pyrimidin-4-yl)-1-methylcyclohexan-1-ol (30 mg, 48% yield) as white solid (¹H NMR (400 MHz, CDCl₃) δ=8.50 (s, 1H), 7.95 (d, J=9.6 Hz, 1H), 7.28 (s, 2H), 7.26-7.21 (m, 2H), 7.21-7.17 (m, 2H), 7.03 (s, 1H), 4.08 (s, 2H), 2.94-2.83 (m, 1H), 2.28 (s, 3H), 1.87-1.76 (m, 6H), 1.65-1.53 (m, 3H), 1.34 (s, 3H)) and (1r,4r)-4-(2-((4-(benzylthio)-2-methylphenyl)amino)-5-(trifluoromethyl)pyrimidin-4-yl)-1-methylcyclohexan-1-ol (60 mg, 96% yield) as white solid. ¹H NMR (400 MHz, CDCl₃) δ 8.51 (s, 1H), 7.95 (d, J=8.4 Hz, 1H), 7.30-7.28 (m, 3H), 7.26-7.20 (m, 2H), 7.17 (s, 1H), 7.05 (s, 1H), 5.30 (s, 1H), 4.09 (s, 2H), 3.49 (s, 1H), 2.84 (t, J=11.6 Hz, 1H), 2.28 (s, 3H), 1.84-1.75 (m, 2H), 1.65 (d, J=12.4 Hz, 2H), 1.59-1.49 (m, 2H), 1.29 (s, 3H), 0.91-0.82 (m, 2H). The absolute stereochemistry of the diastereomers was confirmed by 2D NMR.

### Synthesis of Tert-butyl ((1r,4r)-4-(piperazin-1-ylmethyl)cyclohexyl)carbamate (Intermediate SZ)

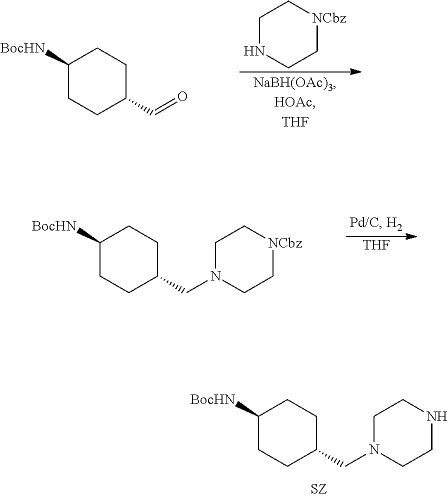 [SMILES: CC(C)(C)OC(=O)N[C@H]1CC[C@H](C=O)CC1 ; O=C(OCc1ccccc1)N1CCNCC1 ; CC(C)(C)OC(=O)N[C@H]1CC[C@H](CN2CCN(C(=O)OCc3ccccc3)CC2)CC1 ; CC(C)(C)OC(=O)N[C@H]1CC[C@H](CN2CCNCC2)CC1 ; "THF"]

### Step 1—Tert-butyl N-[1-deuterio-2-(4-formylcyclohexoxy)-1-methyl-ethyl]carbamate

To a solution of benzyl piperazine-1-carboxylate (1.16 g, 5.28 mmol, 1.02 mL, CAS #181308-57-6) in THF (5 mL) was added HOAc (132 mg, 2.20 mmol, 125 μL) at 0° C. Then tert-butyl N-(4-formylcyclohexyl)carbamate (1 g, 4.40 mmol, CAS #31166-44-6) in THF (5 mL) was added at 0° C. and the mixture was stirred for 0.5 hr. Next, NaBH(OAc)₃ (1.86 g, 8.80 mmol) was added at 0° C. and the reaction mixture was stirred at 0° C. for 0.5 hr. On completion, the reaction was diluted with H₂O (30 mL) and extracted with EtOAc (60 mL). The organic layer was washed with brine (30 mL), dried over with Na₂SO₄, filtered and concentrated in vacuo. The residue was purified by flash silica gel chromatography (ISCO®; 20 g SepaFlash® Silica Flash Column, Eluent of 0˜82% Ethyl acetate/Petroleum ethergradient @ 80 mL/min) to give the title compound (1.6 mg, 84% yield) as yellow oil. LC-MS (ESI⁺) m/z 432.1 (M+H)⁺.

### Step 2—Tert-butyl ((1r,4r)-4-(piperazin-1-ylmethyl)cyclohexyl)carbamate

To a suspension of Pd/C (1.60 g, 1.50 mmol, 10 wt %) in THF (20 mL) was added a solution of benzyl 4-[[4-(tert-butoxycarbonylamino)cyclohexyl]methyl]piperazine-1-carboxylate (1.6 g, 3.71 mmol) in THF (10 mL) under N₂. The suspension was degassed under vacuum and purged with H₂ several times. The mixture was then stirred under H₂ (15 psi) at rt for 15 hrs. On completion, the mixture was filtered and concentrated in vacuo to give the title compound (1.1 g, 99% yield) as a yellow oil. ¹H NMR (400 MHz, CDCl₃) δ 4.41 (s, 1H), 3.81-3.69 (m, 1H), 3.52-2.94 (m, 4H), 2.60 (s, 2H), 2.19 (s, 1H), 2.08-1.95 (m, 8H), 1.88-1.75 (m, 2H), 1.44 (s, 9H), 1.13-0.90 (m, 3H).

### Synthesis of Tert-butyl 4-(4-amino-3-methyl-phenyl)sulfonylpiperidine-1-carboxylate (Intermediate TA)

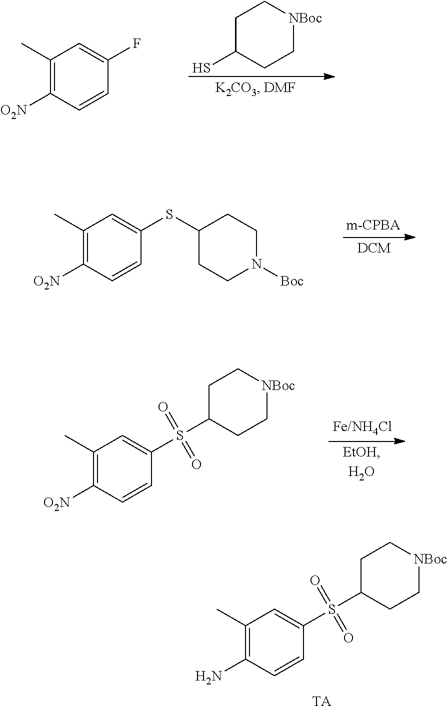

### Step 1—Tert-butyl 4-(3-methyl-4-nitro-phenyl)sulfanylpiperidine-1-carboxylate

To a solution of 4-fluoro-2-methyl-1-nitro-benzene (2.14 g, 13.8 mmol, CAS #446-33-3) and tert-butyl 4-sulfanylpiperidine-1-carboxylate (2.50 g, 11.5 mmol, CAS #134464-79-2) in DMF (30 mL) was added K₂CO₃ (3.18 g, 23.0 mmol), then the mixture was stirred at 25° C. for 8 hrs. On completion, the mixture was diluted with water (30 mL) and extracted with EA (20 mL×3). The combined organic layer was dried over anhydrous Na₂SO₄, filtered and concentrated in vacuo to give the residue. The residue was purified by column chromatography (SiO₂, PE:EA=15:1 to 7:1) to give the title compound (3.60 g, 88% yield) as yellow solid. ¹H NMR (400 MHz, DMSO-d₆) δ 7.96 (d, J=8.8 Hz, 1H), 7.44 (d, J=1.6 Hz, 1H), 7.39-7.38 (m, 1H), 3.83 (d, J=13.6 Hz, 2H), 3.78-3.70 (m, 1H), 3.09-2.92 (m, 2H), 2.52 (s, 3H), 2.02-1.90 (m, 2H), 1.46-1.40 (m, 2H), 1.39 (s, 9H).

### Step 2—Tert-butyl 4-(3-methyl-4-nitro-phenyl)sulfonylpiperidine-1-carboxylate

To a solution of tert-butyl 4-(3-methyl-4-nitro-phenyl)sulfanylpiperidine-1-carboxylate (1.00 g, 2.84 mmol) in DCM (10 mL) was added MCPBA (2.45 g, 14.1 mmol) at 0 C, then the mixture was stirred at 25° C. for 1 hr. On completion, the mixture was quenched with Na₂SO₃ (10 mL) and Na₂CO₃ (8 mL) at 0° C., diluted with water (8 mL) and extracted with DCM (8 mL×3). The combined organic layer was dried over anhydrous Na₂SO₄, filtered and concentrated in vacuo to give the residue. The residue was purified by column chromatography (SiO₂, PE:EA=4:1 to 1:1) to give the title compound (900 mg, 82% yield) as yellow solid. ¹H NMR (400 MHz, DMSO-d₆) δ 8.20 (d, J=8.4 Hz, 1H), 8.01 (d, J=1.2 Hz, 1H), 7.90-7.89 (m, 1H), 4.01 (d, J=11.6 Hz, 2H), 3.73-3.54 (m, 1H), 2.75-2.64 (m, 2H), 2.58 (s, 3H), 1.84 (d, J=11.6 Hz, 2H), 1.45-1.38 (m, 2H), 1.37 (s, 9H).

### Step 3—Tert-butyl 4-(4-amino-3-methyl-phenyl) sulfonylpiperidine-1-carboxylate

To a solution of tert-butyl 4-(3-methyl-4-nitro-phenyl)sulfonylpiperidine-1-carboxylate (0.400 g, 1.04 mmol) in EtOH (10 mL) and H₂O (2 mL) was added Fe (348 mg, 6.24 mmol) and NH₄Cl (556 mg, 10.4 mmol). The reaction mixture was then stirred at 80° C. for 2 hrs. On completion, the reaction mixture was filtered and filtrate was concentrated in vacuo and diluted with water (10 mL), then the residue was extracted with EA (3×20 mL). The combined organic layer was dried over Na₂SO₄, filtered and filtrate was concentrated in vacuo. The residue was purified by column chromatography to give the title compound (300 mg, 81% yield) as yellow solid. LC-MS (ESI⁺) m/z 298.9 (M−56)⁺.

### Synthesis of Benzyl 4-(((1s,4s)-4-((tert-butoxycarbonyl)amino)-1-fluorocyclohexyl)methyl)piperazine-1-carboxylate (Intermediate TB) and benzyl 4-(((1r,4r)-4-((tert-butoxycarbonyl)amino)-1-fluorocyclohexyl)methyl)piperazine-1-carboxylate (Intermediate TC)

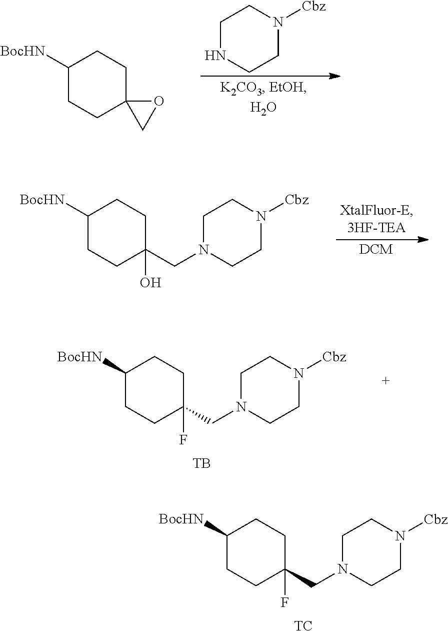

### Step 1—Benzyl 4-[[4-(tert-butoxycarbonylamino)-1-hydroxy-cyclohexyl]methyl]piperazine-1-carboxylate

To a solution of tert-butyl N-(1-oxaspiro[2.5]octan-6-yl)carbamate (4.5 g, 19.8 mmol, CAS #959704-59-7) and benzyl piperazine-1-carboxylate (6.54 g, 29.7 mmol, 5.73 mL, CAS #31166-44-6) in EtOH (40 mL) and H₂O (20 mL) was added K₂CO₃ (2.74 g, 19.8 mmol), then the mixture was stirred at 95° C. for 0.5 hr. On completion, the reaction was concentrated in vacuo, diluted with EA (60 mL) and washed with water (60 mL). The organic layer was dried over Na₂SO₄, filtered and the filtrate was concentrated in vacuo. The reaction was purified by column chromatography (SiO₂, PE:EA=5:1 to 0:1, Rf=0.3) to give the title compound (8 g, 17.8 mmol, 90% yield) as yellow oil. ¹H NMR (400 MHz, DMSO-d₆) δ 7.40-7.30 (m, 5H), 5.07 (s, 2H), 3.84 (s, 1H), 3.37 (s, 4H), 3.17-3.05 (m, 1H), 2.47 (d, J=4.4 Hz, 4H), 2.18 (s, 2H), 1.56-1.46 (m, 6H), 1.37 (s, 9H), 1.32-1.24 (m, 2H). LC-MS (ESI⁺) m/z 448.1 (M+H)⁺.

### Step 2—Benzyl 4-(((1s,4s)-4-((tert-butoxycarbonyl)amino)-1-fluorocyclohexyl)methyl)piperazine-1-carboxylate and benzyl 4-(((1r,4r)-4-((tert-butoxycarbonyl)amino)-1-fluorocyclohexyl)methyl)piperazine-1-carboxylate

To a solution of benzyl 4-[[4-(tert-butoxycarbonylamino)-1-hydroxy-cyclohexyl]methyl]piperazine-1-carboxylate (4.5 g, 10.0 mmol) in DCM (50 mL) was added N,N-diethylethanamine trihydrofluoride (3.24 g, 20.1 mmol, 3.28 mL) and (difluoro-sulfanylidene)-diethyl-ammonium tetrafluoroborate (4.60 g, 20.1 mmol) at 0° C., the mixture was stirred at rt for 4 hrs. On completion, the reaction mixture was diluted with water (50 mL) and extracted with DCM (50 mL×2). The combined organic layers were dried over Na₂SO₄, filtered and concentrated in vacuo. The crude product was purified by column chromatography (SiO₂, PE:EA=50:1 to 1:1) twice to give benzyl 4-(((1s,4s)-4-((tert-butoxycarbonyl)amino)-1-fluorocyclohexyl)methyl)piperazine-1-carboxylate (390 mg, 4% yield, peak 1) as yellow solid (¹H NMR (400 MHz, CDCl₃-d) 6 7.41-7.31 (m, 5H), 5.14 (s, 2H), 4.48 (d, J=4.4 Hz, 1H), 3.74-3.63 (m, 1H), 3.50 (s, 4H), 2.57-242 (m, 6H), 1.98-1.91 (m, 2H), 1.81-1.73 (m, 4H), 1.45 (s, 9H), 1.44-1.43 (m, 2H). LC-MS (ESI⁺) m/z 450.4 (M+H)+) and benzyl 4-(((1r,4r)-4-((tert-butoxycarbonyl)amino)-1-fluorocyclohexyl)methyl)piperazine-1-carboxylate (210 mg, 2% yield) as yellow solid (¹H NMR (400 MHz, CDCl₃-d) 6 7.44-7.30 (m, 5H), 5.13 (s, 2H), 4.52-4.40 (m, 1H), 3.49 (s, 5H), 2.58-2.35 (m, 6H), 2.09-1.98 (m, 2H), 1.86 (d, J=1.6 Hz, 2H), 1.45 (s, 11H), 1.40 (d, J=8.8 Hz, 2H). LC-MS (ESI⁺) m/z 450.5 (M+H)+). The absolute stereochemistry of the diastereomers was assigned arbitrarily.

### Synthesis of Tert-butyl N-(4-formylnorbornan-1-yl)carbamate (Intermediate TD)

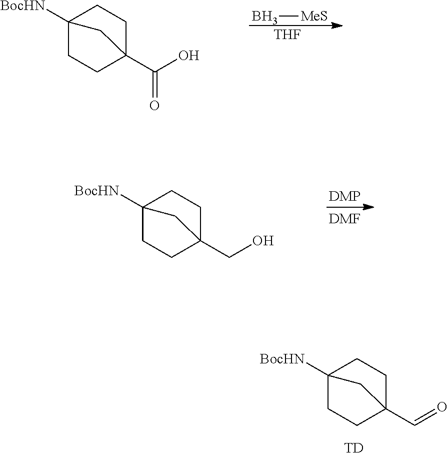

### Step 1—Tert-butyl N-[4-(hydroxymethyl)norbornan-1-yl]carbamate

To a solution of 4-(tert-butoxycarbonylamino)norbornane-1-carboxylic acid (1.90 g, 7.44 mmol, CAS #1201186-86-8) in THF (20 mL) was added BH₃-Me₂S (1.70 g, 22.3 mmol) at 0° C. Then the mixture was stirred at 25° C. for 16 hours under N₂ atmosphere. On completion, the reaction mixture was added into (MeOH 5 mL) dropwise. The crude product was purified by column chromatography on silica gel eluted with petroleum ether/ethyl acetate=1:0 to 1:1 to give the title compound (1.60 g, 80% yield) as a white solid. ¹H NMR (400 MHz, DMSO-d₆) δ 6.88 (s, 1H), 4.39 (t, J=5.2 Hz, 1H), 3.36 (d, J=5.2 Hz, 2H), 1.74 (s, 2H), 1.64-1.49 (m, 4H), 1.42 (s, 2H), 1.37 (s, 9H), 1.26-1.18 (m, 2H).

### Step 2—Tert-butyl N-(4-formylnorbornan-1-yl)carbamate

To a solution of tert-butyl N-[4-(hydroxymethyl)norbornan-1-yl]carbamate (1.50 g, 6.22 mmol) in DMF (5 mL) was added DMP (3.16 g, 7.46 mmol). Then the mixture was stirred at 25° C. for 1 hr. On completion, the reaction mixture was quenched by adding it to H₂O solution (5 mL). The aqueous layer was extracted with ethyl acetate (5 ml×3). The crude product was purified by column chromatography on silica gel eluted with petroleum ether/ethyl acetate=1:0 to 1:1 to give the title compound (800 mg, 48% yield) as a yellow solid. ¹H NMR (400 MHz, DMSO-d₆) δ 9.71 (s, 1H), 4.04 (q, J=7.2 Hz, 1H), 1.92 (s, 2H), 1.90-1.80 (m, 2H), 1.75 (d, J=5.2 Hz, 2H), 1.63-1.55 (m, 2H), 1.38 (d, J=1.2 Hz, 9H), 1.18 (t, J=7.2 Hz, 2H).

### Synthesis of Tert-butyl ((1r,4r)-4-(piperazin-1-yl)cyclohexyl)carbamate (Intermediate TE)

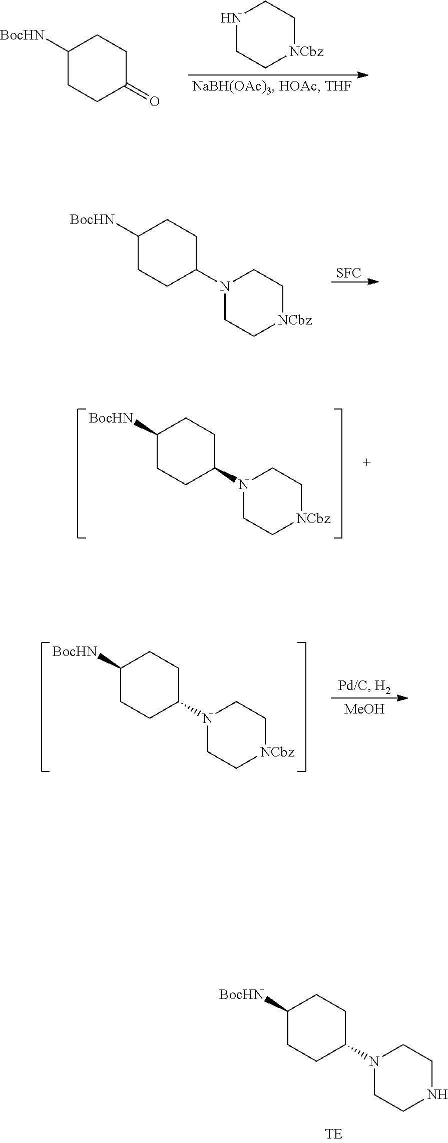

### Step 1—Benzyl 4-[4-(tert-butoxycarbonylamino)cyclohexyl]piperazine-1-carboxylate

To a solution of benzyl piperazine-1-carboxylate (5.68 g, 25.7 mmol, CAS #31166-44-6) and tert-butyl N-(4-oxocyclohexyl)carbamate (5 g, 23.4 mmol, CAS #179321-49-4) in THF (40 mL) was added HOAc (1.41 g, 23.4 mmol) and the mixture was stirred at 25° C. for 0.5 hr. Then NaBH(OAc)₃ (5.96 g, 28.1 mmol) was added into the mixture at 25° C. for 2 hrs. On completion, the mixture was quenched with H₂O (6 ml) and concentrated in vacuo to give residue. The residue was purified by column chromatography (SiO₂, Petroleum ether/Ethyl acetate=10/1 to 0/1) to give the title compound (9 g, 91% yield) as white solid. ¹H NMR (400 MHz, DMSO-d₆) δ 7.40-7.30 (m, 5H), 5.07 (d, J=3.6 Hz, 2H), 3.54-3.43 (m, 1H), 3.36 (s, 4H), 3.21-2.99 (m, 1H), 2.43 (s, 4H), 2.24-2.12 (m, 1H), 1.83-1.70 (m, 2H), 1.65-1.57 (m, 2H), 1.42 (d, J=8.4 Hz, 2H), 1.39-1.35 (m, 9H), 1.17-1.09 (m, 2H).

### Step 2—Benzyl 4-((1s,4s)-4-((tert-butoxycarbonyl)amino)cyclohexyl)piperazine-1-carboxylate and benzyl 4-((1r,4r)-4-((tert-butoxycarbonyl)amino)cyclohexyl)piperazine-1-carboxylate

Benzyl 4-[4-(tert-butoxycarbonylamino)cyclohexyl]piperazine-1-carboxylate (12 g) was separated by SFC (column: DAICEL CHIRALPAK AD (250 mm*30 mm, 10 um); mobile phase: [CO2-MeOH (0.1% NH3H2O)]; B %:20%, isocratic elution mode) to give benzyl 4-((1s,4s)-4-((tert-butoxycarbonyl)amino)cyclohexyl)piperazine-1-carboxylate (5 g, 41% yield, peak 1) as white solid (¹H NMR (400 MHz, CHLOROFORM-d) δ 7.42-7.31 (m, 5H), 5.15 (s, 2H), 4.71-4.54 (m, 1H), 3.79-3.66 (m, 1H), 3.53 (s, 4H), 2.52 (s, 4H), 2.32-2.17 (m, 1H), 1.80 (d, J=9.6 Hz, 2H), 1.68 (s, 2H), 1.61-1.50 (m, 4H), 1.46 (s, 9H), LC-MS (ESI⁺) m/z 418.3 (M+H)+) and benzyl 4-((1r,4r)-4-((tert-butoxycarbonyl)amino)cyclohexyl)piperazine-1-carboxylate (5 g, 41% yield, peak 2) as white solid (¹H NMR (400 MHz, CHLOROFORM-d) δ 7.37-7.21 (m, 4H), 5.05 (s, 2H), 4.37-4.24 (m, 1H), 3.46-3.41 (m, 4H), 3.34-3.18 (m, 1H), 2.44 (s, 4H), 2.19 (s, 1H), 1.99 (d, J=11.2 Hz, 2H), 1.80 (d, J=12.4 Hz, 2H), 1.36 (s, 9H), 1.26 (d, J=14.0 Hz, 2H), 1.04 (dd, J=2.0, 12.4 Hz, 2H); LC-MS (ESI⁺) m/z 418.3 (M+H)+). The absolute stereochemistry of the diastereomers was confirmed by 2D NMR.

### Step 3—Tert-butyl ((1r,4r)-4-(piperazin-1-yl)cyclohexyl)carbamate

To a solution of benzyl 4-[4-(tert-butoxycarbonylamino)cyclohexyl]piperazine-1-carboxylate (1 g, 2.39 mmol) in MeOH (20 mL) was added Pd/C (500 mg, 469 μmol, 10 wt %) under N₂. The suspension was degassed under vacuum and purged with H₂ several times. The mixture was then stirred under H₂ (15 psi) at 25° C. for 2 hrs. On completion, the mixture was filtered and concentrated in vacuo to give the title compound (7.0 g, 71% yield) yellow oil. LC-MS (ESI⁺) m/z 284.1 (M+H)⁺.

### Synthesis of [3-Fluoro-N-[(3S,4R)-3-fluoro-4-piperidyl]-4-[[4-[(3S)-3-hydroxy-3-methyl-1-piperidyl]-5-(trifluoromethyl)pyrimidin-2-yl]amino]benzenesulfonamide (Intermediate TF)

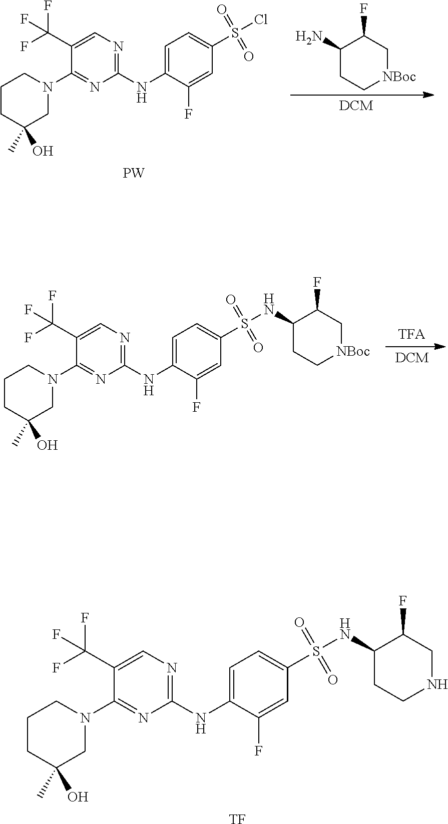

### Step 1—Tert-butyl (3S,4R)-3-fluoro-4-[[3-fluoro-4-[[4-[(3S)-3-hydroxy-3-methyl-1-piperidyl]-5-(trifluoromethyl)pyrimidin-2-yl]amino]phenyl]sulfonylamino]piperidine-1-carboxylate

To a solution of 3-fluoro-4-[[4-[(3S)-3-hydroxy-3-methyl-1-piperidyl]-5-(trifluoromethyl)pyrimidin-2-yl]amino]benzenesulfonyl chloride (200 mg, 426 μmol, Intermediate PW) in DCM (5 mL) was added tert-butyl (3S,4R)-4-amino-3-fluoro-piperidine-1-carboxylate (279 mg, 1.28 mmol, CAS #907544-20-1), then the reaction was stirred at 25° C. for 0.2 hr. On completion, the reaction was concentrated in vacuo and purified by prep-HPLC (column: Phenomenex luna C18 150*25 mm*10 um; mobile phase: [water (FA)-ACN]; gradient: 49%-79% B over 10 min) to give the title compound (220 mg, 79% yield) as white solid. ¹H NMR (400 MHz, DMSO-d₆) δ 9.60 (s, 1H), 8.40 (s, 1H), 8.20-8.00 (m, 2H), 7.75-7.56 (m, 2H), 4.49 (s, 1H), 4.22-3.99 (m, 1H), 3.84 (s, 1H), 3.73-3.63 (m, 1H), 3.57-3.43 (m, 2H), 3.30-3.22 (m, 2H), 3.19-2.86 (m, 2H), 2.79-2.66 (m, 1H), 1.79 (d, J=4.4 Hz, 1H), 1.58 (s, 2H), 1.52-1.42 (m, 2H), 1.36 (s, 9H), 1.25 (d, J=10.0 Hz, 1H), 1.05 (s, 3H).

### Step 2—3-Fluoro-N-[(3S,4R)-3-fluoro-4-piperidyl]-4-[[4-[(3S)-3-hydroxy-3-methyl-1-piperidyl]-5-(trifluoromethyl)pyrimidin-2-yl]amino]benzenesulfonamide

A solution of tert-butyl (3S,4R)-3-fluoro-4-[[3-fluoro-4-[[4-[(3S)-3-hydroxy-3-methyl-1-piperidyl]-5-(trifluoromethyl)pyrimidin-2-yl]amino]phenyl]sulfonylamino]piperidine-1-carboxylate (90 mg, 138 mol) in DCM (1.5 mL) and TFA (0.5 mL) was stirred at 30° C. for 0.2 hr. On completion, the reaction was concentrated in vacuo to give the title compound (90 mg, 97% yield, TFA) as yellow oil. LC-MS (ESI⁺) m/z 551.1 (M+H)⁺.

### Synthesis of 1-[4-(2,6-Dioxo-3-piperidyl)-3-fluoro-phenyl]piperidine-4-carbaldehyde (Intermediate TG)

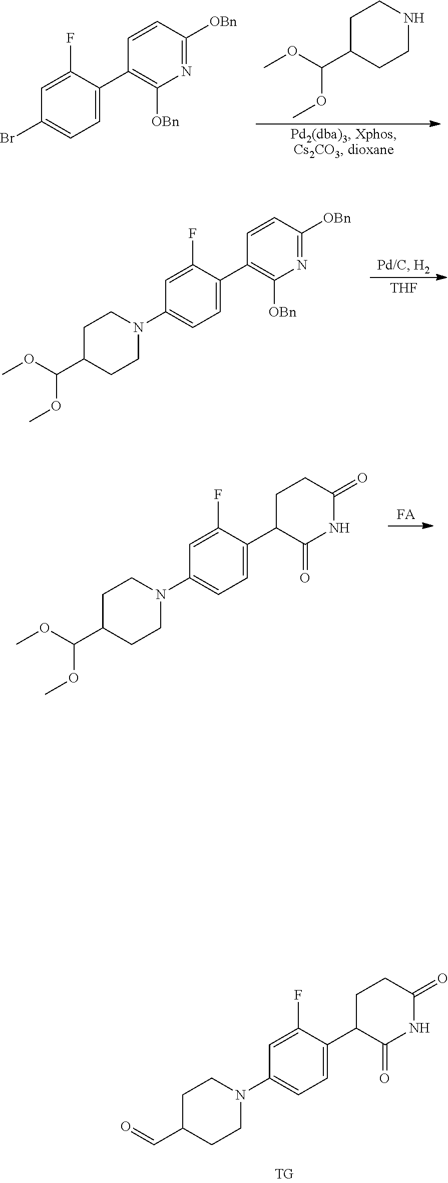

### Step 1—2,6-Dibenzyloxy-3-[4-[4-(dimethoxymethyl)-1-piperidyl]-2-fluoro-phenyl]pyridine

A mixture of 2,6-dibenzyloxy-3-(4-bromo-2-fluoro-phenyl)pyridine (2 g, 4 mmol), 4-(dimethoxymethyl)piperidine (1.03 g, 6.46 mmol, CAS #188646-83-5), XPhos (205 mg, 430 mol), Cs₂CO₃ (2.81 g, 8.61 mmol) and Pd₂(dba)₃ (394 mg, 430 μmol) in dioxane (25 mL) was degassed and purged with N₂ three times. Then the mixture was stirred at 110° C. for 12 hrs under N₂ atmosphere. On completion, the mixture was filtered directly and filtrate was diluted with EA (10 mL). The organic layer was washed with brine (10 mL×2), dried over Na₂SO₄ and concentrated in vacuo. The residue was purified by column chromatography (SiO₂, Petroleum ether/Ethyl acetate=10:1 to 5:1) to give the title compound (1.6 g, 68% yield) as a white solid. ¹H NMR (400 MHz, DMSO-d₆) δ 7.57 (d, J=8.0 Hz, 1H), 7.46-7.25 (m, 10H), 7.18 (t, J=8.8 Hz, 1H), 6.80-6.71 (m, 2H), 6.50 (d, J=8.0 Hz, 1H), 5.35 (d, J=5.6 Hz, 4H), 4.08 (d, J=6.8 Hz, 1H), 3.77 (d, J=12.4 Hz, 2H), 3.32 (s, 3H), 3.27 (s, 3H), 2.67 (t, J=11.6 Hz, 2H), 1.81-1.63 (m, 3H), 1.37-1.21 (m, 2H).

### Step 2—3-[4-[4-(Dimethoxymethyl)-1-piperidyl]-2-fluoro-phenyl]piperidine-2,6-dione

To a solution of 2,6-dibenzyloxy-3-[4-[4-(dimethoxymethyl)-1-piperidyl]-2-fluoro-phenyl]pyridine (1.6 g, 3.0 mmol) in THF (16 mL) was added Pd/C (1.5 g, 1.41 mmol, 10 wt %) under N₂ atmosphere. The suspension was degassed and purged with H₂ three times. The mixture was stirred under H₂ (15 Psi) at 20° C. for 12 hrs. On completion, the reaction was filtered and the filtrate was concentrated in vacuo to give the title compound (1 g, 93% yield) as a white solid. ¹H NMR (400 MHz, DMSO-d₆) δ 10.79 (s, 1H), 7.05 (t, J=8.8 Hz, 1H), 6.71 (s, 1H), 6.68 (s, 1H), 4.08 (d, J=6.8 Hz, 1H), 3.87 (dd, J=4.8, 12.4 Hz, 1H), 3.74 (s, 1H), 3.71 (s, 1H), 3.27 (s, 6H), 2.78-2.57 (m, 4H), 2.13 (dq, J=4.0, 12.0 Hz, 1H), 2.01-1.90 (m, 1H), 1.80-1.72 (m, 1H), 1.69 (d, J=12.4 Hz, 2H), 1.32-1.23 (m, 2H).

### Step 3—1-[4-(2,6-Dioxo-3-piperidyl)-3-fluoro-phenyl]piperidine-4-carbaldehyde

A solution of 3-[4-[4-(dimethoxymethyl)-1-piperidyl]-2-fluoro-phenyl]piperidine-2,6-dione (55 mg, 150 μmol) in HCOOH (0.5 mL) was stirred at 70° C. for 0.5 hr. On completion, the reaction was concentrated in vacuo to give the title compound (50 mg, 90% yield, FA) as yellow oil. LC-MS (ESI⁺) m/z 319.2 (M+H)⁺.

### Synthesis of 3-Fluoro-4-[[4-[(3S)-3-hydroxy-3-methyl-1-piperidyl]-5-(trifluoromethyl)pyrimidin-2-yl]amino]-N-[(3S,4R)-3-methyl-4-piperidyl]benzenesulfonamide (Intermediate TH)

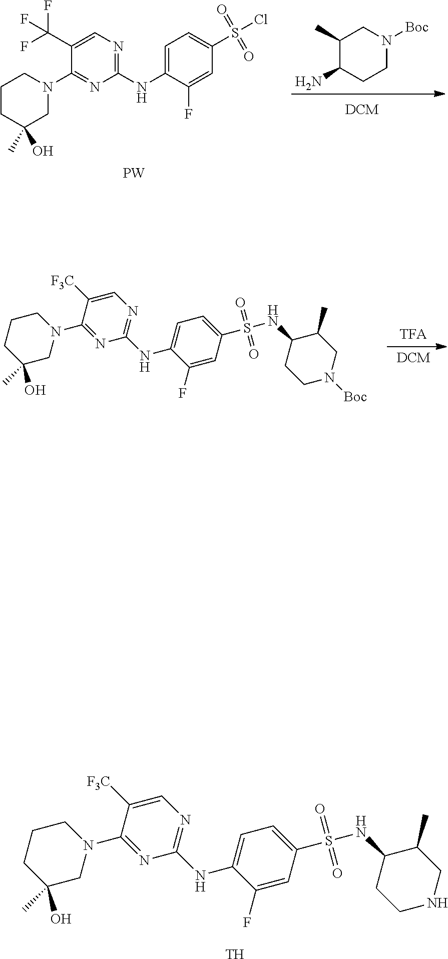

### Step 1—Tert-butyl (3S,4R)-4-[[3-fluoro-4-[[4-[(3S)-3-hydroxy-3-methyl-1-piperidyl]-5-(trifluoromethyl)pyrimidin-2-yl]amino]phenyl]sulfonylamino]-3-methyl-piperidine-1-carboxylate

To a solution of tert-butyl (3S,4R)-4-amino-3-methyl-piperidine-1-carboxylate (118 mg, 554 μmol, CAS #1290191-72-8) in DCM (2 mL) was added 3-fluoro-4-[[4-[(3S)-3-hydroxy-3-methyl-1-piperidyl]-5-(trifluoromethyl) pyrimidin-2-yl]amino]benzenesulfonyl chloride (130 mg, 277 μmol, Intermediate PW), then the mixture was stirred at 25° C. for 12 hrs. On completion, the reaction mixture was concentrated in vacuo. The residue was purified by prep-HPLC (column: Phenomenex luna C18 150*25 mm*10 um; mobile phase: [water (FA)-ACN]; gradient: 53%-83% B over 10 min) to give the title compound (129 mg, 71% yield) as white solid. ¹H NMR (400 MHz, DMSO-d₆) δ 9.57 (s, 1H), 8.39 (s, 1H), 8.10 (t, J=8.4 Hz, 1H), 7.76 (d, J=7.6 Hz, 1H), 7.65-7.59 (m, 2H), 4.47 (s, 1H), 3.69-3.61 (m, 1H), 3.46-3.39 (m, 2H), 3.31-3.24 (m, 4H), 3.14 (s, 1H), 1.83-1.68 (m, 2H), 1.60-1.53 (m, 2H), 1.45 (dd, J=2.4, 6.4 Hz, 1H), 1.35 (s, 11H), 1.29 (d, J=1.2 Hz, 1H), 1.04 (s, 3H), 0.69 (d, J=6.8 Hz, 3H); LC-MS (ESI⁺) m/z 647.2 (M+H)⁺.

### Step 2—3-Fluoro-4-[[4-[(3S)-3-hydroxy-3-methyl-1-piperidyl]-5-(trifluoromethyl)pyrimidin-2-yl]amino]-N-[(3S,4R)-3-methyl-4-piperidyl]benzenesulfonamide

To a solution of tert-butyl (3S,4R)-4-[[3-fluoro-4-[[4-[(3S)-3-hydroxy-3-methyl-1-piperidyl]-5-(trifluoromethyl)pyrimidin-2-yl]amino]phenyl]sulfonylamino]-3-methyl-piperidine-1-carboxylate (80 mg, 123 μmol) in DCM (1 mL) was added TFA (767 mg, 6.73 mmol, 0.5 mL), then the mixture was stirred at 25° C. for 0.5 hr. On completion, the reaction mixture was concentrated in vacuo to give the title compound (80 mg, 97% yield, TFA) as yellow oil. LC-MS (ESI⁺) m/z 547.2 (M+H)⁺.

### Synthesis of 3-[4-[(3R)-4-[(4-aminocyclohexyl)methyl]-3-(methoxymethyl)piperazin-1-yl]-2-fluoro -phenyl]piperidine-2,6-dione (Intermediate TI)

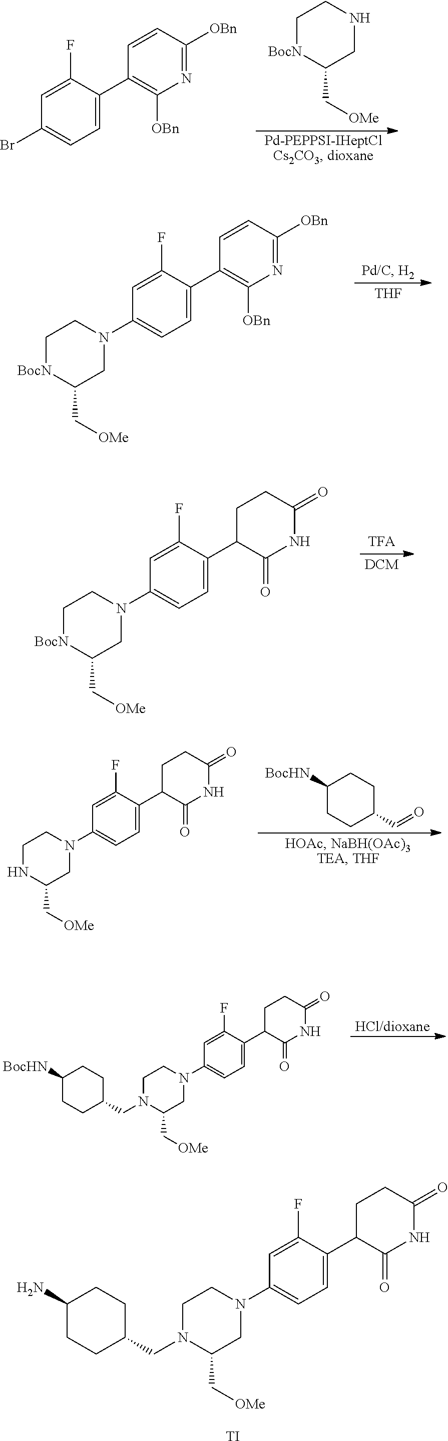

### Step 1—Tert-butyl (2R)-4-[4-(2,6-dibenzyloxy-3-pyridyl)-3-fluoro-phenyl]-2-(methoxymethyl) piperazine-1-carboxylate

To a solution of 2,6-dibenzyloxy-3-(4-bromo-2-fluoro-phenyl)pyridine (2.00 g, 4.31 mmol, synthesized via Step 1 of Intermediate PO) and tert-butyl (2R)-2-(methoxymethyl)piperazine-1-carboxylate (1.49 g, 6.46 mmol, CAS #1023301-73-6), and Cs₂CO₃ (2.81 g, 8.61 mmol) in dioxane (20 mL) was added Pd-PEPPSI-IHeptCl (419 mg, 431 μmol). Then the mixture was stirred at 100° C. for 1 hr. On completion, the mixture was concentrated in vacuo to give the residue. The residue was purified by column chromatography (SiO₂, PE:EA=100:0 to 9:1) to give the title compound (1.1 g, 41% yield) as a green oil. ¹H NMR (400 MHz, DMSO-d₆) δ 7.57 (d, J=8.0 Hz, 1H), 7.45-7.18 (m, 11H), 6.74 (d, J=11.6 Hz, 2H), 6.51 (d, J=8.0 Hz, 1H), 5.35 (d, J=3.2 Hz, 4H), 4.21 (s, 1H), 3.86-3.80 (m, 1H), 3.75-3.59 (m, 2H), 3.56-3.46 (m, 1H), 3.44-3.37 (m, 1H), 3.26 (s, 3H), 3.16-3.02 (m, 1H), 2.86-2.84 (m, 1H), 2.75-2.67 (m, 1H), 1.42 (s, 9H); LC-MS (ESI⁺) m/z 614.3 (M+H)⁺.

### Step 2—Tert-butyl (2R)-4-[4-(2,6-dioxo-3-piperidyl)-3-fluoro-phenyl]-2-(methoxymethyl)piperazine -1-carboxylate

To a solution of Pd/C (1.00 g, 940 μmol, 10 wt %) in THF (2 mL) was added tert-butyl (2R)-4-[4-(2,6-dibenzyloxy-3-pyridyl)-3-fluoro-phenyl]-2-(methoxymethyl)piperazine-1-carboxylate (1.00 g, 1.63 mmol) under N₂ atmosphere. Then the mixture was degassed and charged with H₂ for three times and then stirred at 40° C. under 40 psi H₂ for 14 hours. On completion, the reaction was filtered and concentrated in vacuo to give the title compound (550 mg, 77% yield) as a white solid. LC-MS (ESI⁺) m/z 458.1 (M+Na)⁺.

### Step 3—3-[2-Fluoro-4-[(3R)-3-(methoxymethyl)piperazin-1-yl]phenyl]piperidine-2,6-dione

To a solution of tert-butyl (2R)-4-[4-(2,6-dioxo-3-piperidyl)-3-fluoro-phenyl]-2-(methoxymethyl) piperazine-1-carboxylate (330 mg, 758 μmol) in DCM (4 mL) was added TFA (1.10 mL, 14.8 mmol), then the mixture was stirred at 25° C. for 0.5 hr. On completion, the reaction was concentrated in vacuo to give the title compound (290 mg, 85% yield, TFA) as a yellow oil. LC-MS (ESI⁺) m/z 336.0 (M+H)⁺.

### Step 4—Tert-butyl N-[4-[[(2R)-4-[4-(2,6-dioxo-3-piperidyl)-3-fluoro-phenyl]-2-(methoxymethyl)piperazin-1-yl]methyl]cyclohexyl]carbamate

To a solution of 3-[2-fluoro-4-[(3R)-3-(methoxymethyl)piperazin-1-yl]phenyl]piperidine-2,6-dione (290 mg, 645 μmol TFA) in THF (5 mL) was added TEA (89.8 μL, 645 μmol) until the pH=7-9. Then tert-butyl N-(4-formylcyclohexyl)carbamate (220 mg, 967.97 μmol, CAS #181308-57-6) in THF (5 mL) and HOAc (36.9 μL, 645 mol) was added to the mixture and the mixture was stirred at −10° C. for 0.5 hr. After 0.5 hour, NaBH(OAc)₃ (274 mg, 1.29 mmol) was added and the mixture was stirred at −10° C. for 0.5 hr. On completion, the mixture was quenched with water (1 mL) and filtered to give the filtrate. The filtrate was purified by prep-HPLC (column: Phenomenex luna C18 150*25 mm*10 um; mobile phase: [water(FA)-ACN]; gradient:14%-44% B over 15 min) to give the title compound (150 mg, 43% yield) as a white solid. LC-MS (ESI⁺) m/z 370.1 (M+H)⁺. ¹H NMR (400 MHz, DMSO-d₆) δ 10.82 (s, 1H), 7.37-7.02 (m, 1H), 6.98-6.56 (m, 3H), 3.98-3.73 (m, 3H), 3.66-3.63 (m, 1H), 3.56-3.46 (m, 1H), 3.37 (s, 3H), 3.30-3.04 (m, 6H), 3.02-2.78 (m, 2H), 2.76-2.65 (m, 1H), 2.22-2.07 (m, 1H), 2.03-1.88 (m, 2H), 1.87-1.65 (m, 4H), 1.37 (s, 9H), 1.27-0.97 (m, 4H), 0.96-0.67 (m, 1H). LC-MS (ESI⁺) m/z 547.3 (M+H)⁺.

### Step 5—3-[4-[(3R)-4-[(4-aminocyclohexyl)methyl]-3-(methoxymethyl)piperazin-1-yl]-2-fluoro-phenyl]piperidine-2,6-dione

To a solution of tert-butyl N-[4-[[(2R)-4-[4-(2,6-dioxo-3-piperidyl)-3-fluoro-phenyl]-2-(methoxymethyl)piperazin-1-yl]methyl]cyclohexyl]carbamate (110 mg, 201 μmol) in DCM (1 mL) was added HCl/dioxane (2 M, 2 mL), then the mixture was stirred at 40° C. for 1 hr. On completion, the reaction was concentrated in vacuo to give the title compound (90 mg, 92% yield, HCl) as a white solid. LC-MS (ESI⁺) m/z 447.1 (M+H)⁺.

### Synthesis of 3-Fluoro-4-[[4-(4-hydroxy-4-methyl-1-piperidyl)-5-(trifluoromethyl)pyrimidin-2-yl]amino]benzenesulfonyl chloride (Intermediate TJ)

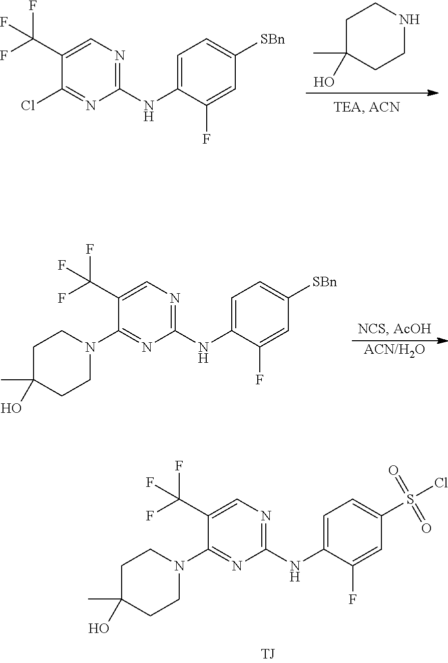

### Step 1—1-[2-(4-benzylsulfanyl-2-fluoro-anilino)-5-(trifluoromethyl)pyrimidin-4-yl]-4-methyl-piperidin-4-ol

To a solution of N-(4-benzylsulfanyl-2-fluoro-phenyl)-4-chloro-5-(trifluoromethyl)pyrimidin-2-amine (400 mg, 966 μmol, synthesized via Step 1 of Intermediate PW) and 4-methylpiperidin-4-ol (161 mg, 1.06 mmol, HCl, CAS #3970-68-1) in ACN (1 mL) was added TEA (293 mg, 2.90 mmol, 403 μL), then the mixture was stirred at 30° C. for 1 hr. On completion, the reaction was concentrated in vacuo and purified by column chromatography (SiO₂, PE:EA=10:1 to PE:EA=2:1, PE:EA=2:1, P1: Rf=0.7) to give the title compound (400 mg, 84% yield) as white solid. ¹H NMR (400 MHz, DMSO-d₆) δ 9.24 (s, 1H), 8.33 (s, 1H), 7.60-7.54 (m, 1H), 7.37-7.29 (m, 4H), 7.28-7.21 (m, 2H), 7.11 (dd, J=2.0, 8.4 Hz, 1H), 4.43 (s, 1H), 4.26 (s, 2H), 3.69 (d, J=13.0 Hz, 2H), 3.40-3.35 (m, 1H), 3.33-3.28 (m, 1H), 1.53-1.46 (m, 4H), 1.14 (s, 3H).

### Step 2—3-Fluoro-4-[[4-(4-hydroxy-4-methyl-1-piperidyl)-5-(trifluoromethyl)pyrimidin-2-yl]amino] benzenesulfonyl chloride

To a solution of 1-[2-(4-benzylsulfanyl-2-fluoro-anilino)-5-(trifluoromethyl)pyrimidin-4-yl]-4-methyl -piperidin-4-ol (200 mg, 406 μmol) in ACN (2 mL) and HOAc (0.2 mL) was added H₂O (731 μg, 40.6 μmol) and NCS (162 mg, 1.22 mmol) in the dark. Then the reaction was stirred at 30° C. for 0.5 hr in the dark. On completion, the reaction mixture was diluted with water (10 mL) and extracted with EA (10 mL×2). The organic layer was dried over Na₂SO₄, filtered and concentrated in vacuo. The reaction was purified by column chromatography (SiO₂, PE:EA=10:1 to 5:1, Rf=0.5) to give the title compound (150 mg, 78% yield) as white solid. LC-MS (ESI⁺) m z 469.1 (M+H)⁺.

### Synthesis of 3-Fluoro-N-[(3S,4R)-3-fluoro-4-piperidyl]-4-[[4-(4-hydroxy-4-methyl-1-piperidyl)-5-(trifluoromethyl)pyrimidin-2-yl]amino]benzenesulfonamide (Intermediate TK)

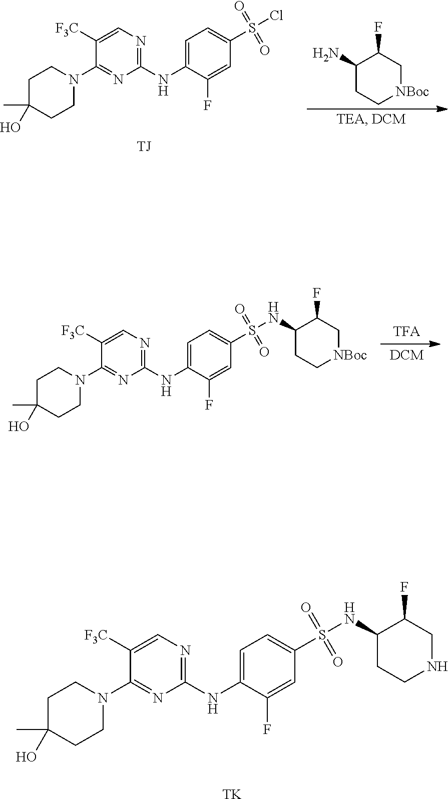

### Step 1—Tert-butyl (3S,4R)-3-fluoro-4-[[3-fluoro-4-[[4-(4-hydroxy-4-methyl-1-piperidyl)-5-(trifluoromethyl)pyrimidin-2-yl]amino]phenyl]sulfonylamino]piperidine-1-carboxylate

To a solution of 3-fluoro-4-[[4-(4-hydroxy-4-methyl-1-piperidyl)-5-(trifluoromethyl)pyrimidin-2-yl] amino]benzenesulfonyl chloride (200 mg, 426 μmol, Intermediate TJ) and tert-butyl (3S,4R)-4-amino-3-fluoro-piperidine-1-carboxylate (465 mg, 2.13 mmol, CAS #907544-20-1) in DCM (3 mL) was added TEA (43.1 mg, 426 μmol), then the reaction was stirred at 30° C. for 3 hrs. On completion, the reaction mixture was diluted with water (10 mL) and extracted with DCM (20 mL×2). The organic layer was dried over Na₂SO₄, filtered and concentrated in vacuo. The reaction was purified by prep-HPLC (column: Phenomenex Luna C18 150*25 mm*10 um; mobile phase: [water (FA)-ACN]; gradient: 48%-78% B over 10 min) to give the title compound (200 mg, 72% yield) white solid. ¹H NMR (400 MHz, DMSO-d₆) δ 9.53 (d, J=8.4 Hz, 1H), 8.44-8.36 (m, 1H), 8.12-8.00 (m, 2H), 7.70-7.59 (m, 2H), 4.65-4.45 (m, 1H), 4.44-4.39 (m, 1H), 4.18-4.02 (m, 1H), 3.91-3.79 (m, 1H), 3.74 (d, J=13.2 Hz, 2H), 3.59-3.45 (m, 1H), 3.43-3.35 (m, 4H), 1.57-1.47 (m, 5H), 1.36 (s, 9H), 1.28-1.21 (m, 1H), 1.14 (s, 3H).

### Step 2—3-Fluoro-N-[(3S,4R)-3-fluoro-4-piperidyl]-4-[[4-(4-hydroxy-4-methyl-1-piperidyl)-5-(trifluoromethyl)pyrimidin-2-yl]amino]benzenesulfonamide

A solution of tert-butyl (3S,4R)-3-fluoro-4-[[3-fluoro-4-[[4-(4-hydroxy-4-methyl-1-piperidyl)-5-(trifluoromethyl)pyrimidin-2-yl]amino]phenyl]sulfonylamino]piperidine-1-carboxylate (50 mg, 76.8 mol) in DCM (0.9 mL) and TFA (0.3 mL) was stirred at 30° C. for 0.5 hr. On completion, the reaction was concentrated in vacuo to give the title compound (50 mg, 97% yield, TFA) as yellow oil. LC-MS (ESI⁺) m/z 551.2 (M+H)⁺.

### Synthesis of 1-[4-(2,6-Dioxo-3-piperidyl)-3,5-difluoro-phenyl]piperidine-4-carbaldehyde (Intermediate TL)

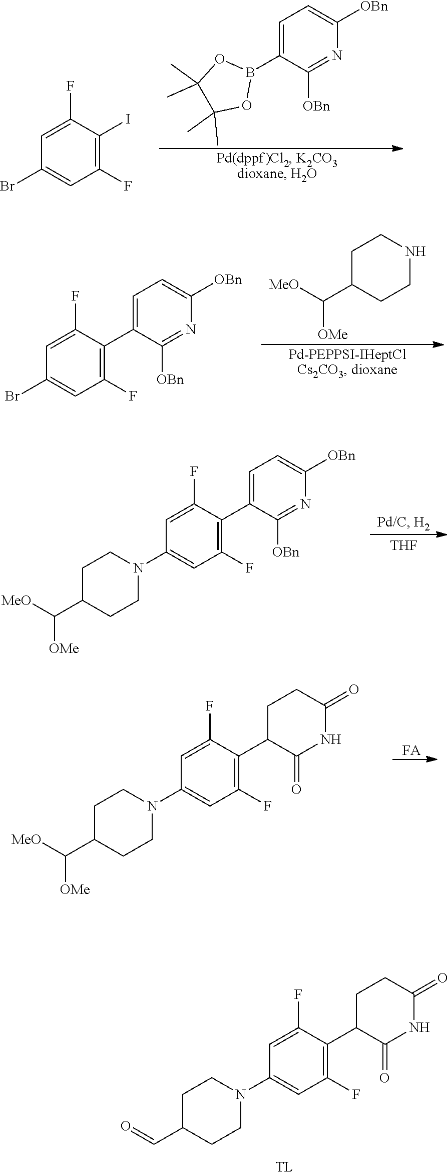

### Step 1—2,6-Dibenzyloxy-3-(4-bromo-2,6-difluoro-phenyl)pyridine

A mixture of 5-bromo-1,3-difluoro-2-iodo-benzene (3 g, 9.41 mmol, CAS #160976-02-3), 2,6-dibenzyloxy-3-(4,4,5,5-tetramethyl-1,3,2-dioxaborolan-2-yl)pyridine (3.14 g, 7.53 mmol, CAS #2152673-80-6), Pd(dppf)Cl₂·CH₂Cl₂ (768 mg, 940 μmol), and K₂CO₃ (3.90 g, 28.2 mmol) in dioxane (30 mL) and H₂O (6 mL) was degassed and purged with N₂ three times. Then the mixture was stirred at 80° C. for 2 hrs under N₂ atmosphere. On completion, the reaction was filtered directly and filtrate was diluted with EA (10 mL). The organic layer was washed with brine (10 mL×2), dried over Na₂SO₄ and concentrated in vacuo. The residue was purified by column chromatography (SiO₂, Petroleum ether/Ethyl acetate=80:1 to 50:1) to give the title compound (1.1 g, 24% yield) as colorless oil. LC-MS (ESI⁺) m/z 482 (M+H)⁺.

### Step 2—2,6-Dibenzyloxy-3-[4-[4-(dimethoxymethyl)-1-piperidyl]-2,6-difluoro-phenyl]pyridine

A mixture of 2,6-dibenzyloxy-3-(4-bromo-2,6-difluoro-phenyl)pyridine (1.65 g, 3.42 mmol), 4-(dimethoxymethyl)piperidine (817 mg, 5.13 mmol, CAS #188646-83-5), Cs₂CO₃ (3.34 g, 10.2 mmol), 1,3-bis [2,6-bis(1-propylbutyl)phenyl]-4,5-dichloro-2H-imidazol-1-ium-2-ide; 3-chloropyridine dichloropalladium (332 mg, 342 μmol) in dioxane (15 mL) was degassed and purged with N₂ three times. Then the mixture was stirred at 110° C. for 4 hrs under N₂ atmosphere. On completion, the reaction was filtered directly and filtrate was diluted with EA (30 mL). The organic layer was washed with H₂O (20 mL×2), dried over Na₂SO₄ and concentrated in vacuo. The residue was purified by column chromatography (SiO₂, Petroleum ether/Ethyl acetate=20:1 to 10:1) to give the title compound (1.2 g, 62% yield) as white solid. ¹H NMR (400 MHz, DMSO-d₆) δ 7.58 (d, J=8.0 Hz, 1H), 7.45-7.23 (m, 10H), 6.66 (d, J=11.6 Hz, 2H), 6.52 (d, J=8.0 Hz, 1H), 5.34 (d, J=8.0 Hz, 4H), 4.07 (d, J=6.8 Hz, 1H), 3.81 (d, J=12.8 Hz, 2H), 3.32 (s, 1H), 3.27 (s, 5H), 2.77-2.67 (m, 2H), 1.84-1.73 (m, 1H), 1.68 (d, J=13.2 Hz, 2H), 1.33-1.21 (m, 2H); LC-MS (ESI⁺) m/z 561.4 (M+H)⁺.

### Step 3—3-[4-[4-(Dimethoxymethyl)-1-piperidyl]-2,6-difluoro-phenyl]piperidine-2,6-dione

To a solution of 2,6-dibenzyloxy-3-[4-[4-(dimethoxymethyl)-1-piperidyl]-2,6-difluoro-phenyl]pyridine (1.2 g, 2.14 mmol) in THF (10 mL) was added Pd/C (1 g, 939 μmol, 10 wt %) under N₂ atmosphere. The suspension was degassed and purged with H₂ three times. The mixture was then stirred under H₂ (15 Psi) at 20° C. for 12 hrs. On completion, the combined organic phase is filtered directly and filtered was concentrated in vacuo to give the title compound (760 mg, 92% yield) as white solid. LC-MS (ESI⁺) m/z 383.2 (M+H)⁺.

### Step 4—1-[4-(2,6-Dioxo-3-piperidyl)-3,5-difluoro-phenyl]piperidine-4-carbaldehyde

A solution of 3-[4-[4-(dimethoxymethyl)-1-piperidyl]-2,6-difluoro-phenyl]piperidine-2,6-dione (44 mg, 115 μmol) in HCOOH (1 mL) was stirred at 80° C. for 1 hr. On completion, the reaction was concentrated in vacuo to give the title compound (43 mg, 97% yield) as colorless oil. LC-MS (ESI⁺) m z 355.2 (M+18)+.

### Synthesis of 1-[4-(2,6-Dioxo-3-piperidyl)phenyl]piperidine-4-carbaldehyde (Intermediate™)

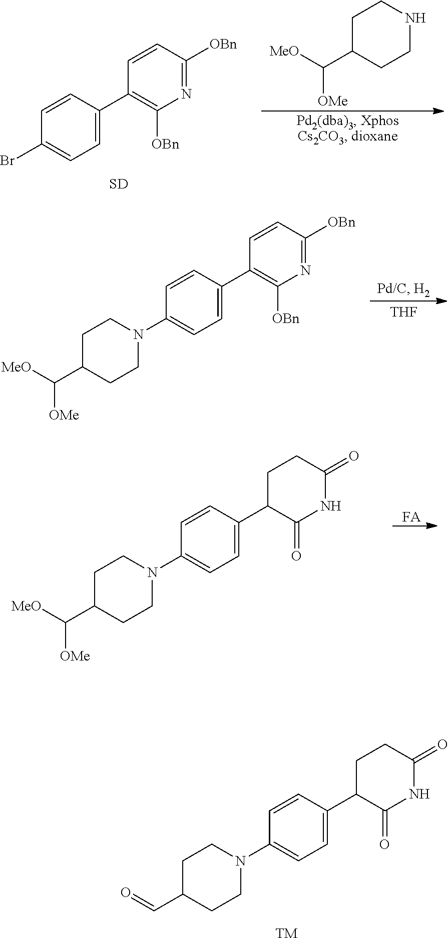

### Step 1—2,6-Dibenzyloxy-3-[4-[4-(dimethoxymethyl)-1-piperidyl]phenyl]pyridine

To a solution of 2,6-dibenzyloxy-3-(4-bromophenyl)pyridine (2 g, 4.48 mmol, Intermediate SD) and 4-(dimethoxymethyl)piperidine (1.07 g, 6.72 mmol, CAS #188646-83-5) in dioxane (15 mL) was added Pd₂(dba)₃ (410 mg, 448 μmol), XPhos (213 mg, 448 μmol) and Cs₂CO₃ (2.92 g, 8.96 mmol), then the mixture was stirred at 100° C. for 16 hrs. On completion, the reaction mixture diluted with water (20 mL) and extracted with EA (80 mL×2). The combined organic layers were washed with water (50 mL×2), dried over Na₂SO₄, filtered and concentrated under reduced pressure. The residue was purified by column chromatography (Silica gel, EA in PE, 8%, v/v) to give the title compound (1.93 g, 82% yield) as white solid. ¹H NMR (400 MHz, DMSO-d₆) δ 7.67 (d, J=8.0 Hz, 1H), 7.48-7.25 (m, 12H), 6.92 (d, J=8.8 Hz, 2H), 6.51 (d, J=8.0 Hz, 1H), 5.75 (s, 1H), 5.37 (d, J=17.2 Hz, 4H), 4.07 (d, J=6.4 Hz, 1H), 3.73 (d, J=12.4 Hz, 2H), 3.27 (s, 6H), 2.69-2.56 (m, 2H), 1.76-1.63 (m, 3H), 1.40-1.23 (m, 2H).

### Step 2—3-[4-[4-(Dimethoxymethyl)-1-piperidyl]phenyl]piperidine-2,6-dione

To a solution of 2,6-dibenzyloxy-3-[4-[4-(dimethoxymethyl)-1-piperidyl]phenyl]pyridine (1.9 g, 3.6 mmol) in THF (20 mL) was added Pd/C (1 g, 939 μmol, 10 wt %), then the mixture was stirred at 25° C. for 16 hrs under H₂. On completion, the reaction was filtered and concentrated in vacuo to give the title compound (1.1 g, 88% yield) as white solid. ¹H NMR (400 MHz, DMSO-d₆) δ 10.76 (s, 1H), 7.03 (d, J=8.4 Hz, 2H), 6.87 (d, J=8.4 Hz, 2H), 4.08 (d, J=6.4 Hz, 1H), 3.74-3.69 (m, 1H), 3.65 (s, 1H), 3.27 (s, 6H), 2.62-2.55 (m, 2H), 2.50 (s, 2H), 2.48-2.41 (m, 1H), 2.17-2.06 (m, 1H), 2.04-1.96 (m, 1H), 1.70 (d, J=9.6 Hz, 3H), 1.38-1.25 (m, 2H).

### Step 3—1-[4-(2,6-Dioxo-3-piperidyl)phenyl]piperidine-4-carbaldehyde

A mixture of 3-[4-[4-(dimethoxymethyl)-1-piperidyl]phenyl]piperidine-2,6-dione (50 mg, 144 μmol) in FA (0.5 mL) was stirred at 80° C. for 1 hr. On completion, the reaction was concentrated in vacuo to give the title compound (49 mg, 98% yield, FA) as yellow solid. LC-MS (ESI⁺) m/z 301.0 (M+H)⁺.

### Synthesis of 1-[3-(2,6-Dioxo-3-piperidyl)-1-methyl-indazol-6-yl]piperidine-4-carbaldehyde (Intermediate TN)

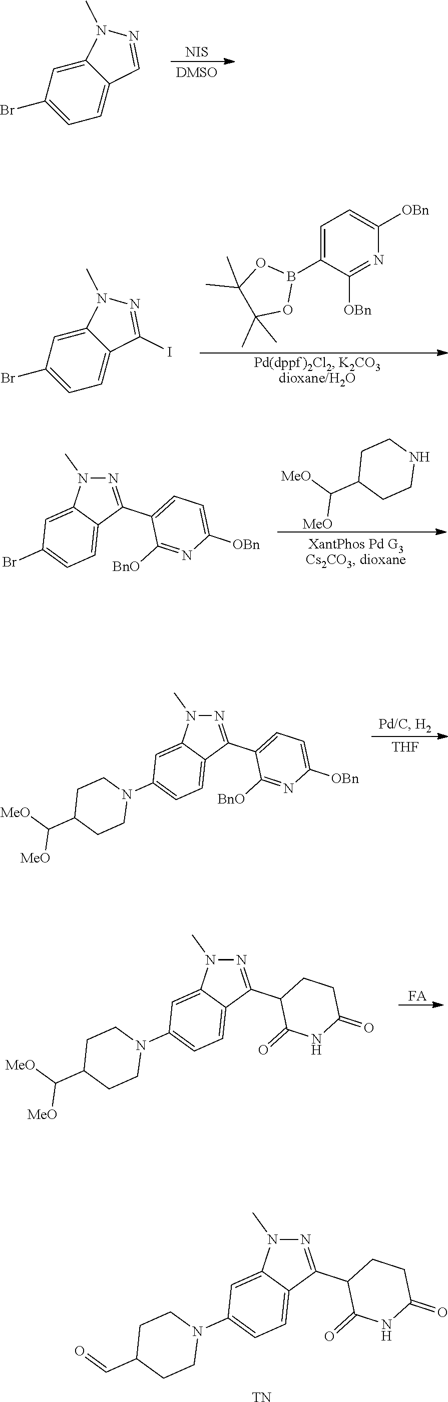

### Step 1—6-Bromo-3-iodo-1-methyl-indazole

To a solution of 6-bromo-1-methyl-indazole (5 g, 23.6 mmol, CAS #590417-94-0) in DMSO (50 mL) was added NIS (6.40 g, 28.4 mmol) at 25° C. under N₂. The reaction was then stirred at 90° C. for 16 hrs under N₂. On completion, the reaction was diluted with EA (200 mL). The organic layer was washed with water (100 mL×3), dried over Na₂SO₄ and concentrated in vacuo. The residue was purified by column chromatography (SiO₂, Petroleum ether/Ethyl acetate=100/1 to 20/1) to give the title compound (7.98 g, 100% yield) as yellow solid. ¹H NMR (400 MHz, DMSO-d₆) δ 8.03 (s, 1H), 7.38-7.29 (m, 2H), 4.05 (s, 3H); LC-MS (ESI⁺) m/z 336.6 (M+H)⁺.

### Step 2—6-Bromo-3-(2,6-dibenzyloxy-3-pyridyl)-1-methyl-indazole

To a solution of 6-bromo-3-iodo-1-methyl-indazole (3 g, 8.90 mmol), 2,6-dibenzyloxy-3-(4,4,5,5-tetramethyl-1,3,2-dioxaborolan-2-yl)pyridine (3.72 g, 8.90 mmol, CAS #2152673-80-6) and Pd(dppf)Cl₂ (6.51 g, 8.90 mmol) in dioxane (30 mL) and H₂O (6 mL) was added K₂CO₃ (3.69 g, 26.7 mmol). The reaction was then stirred at 80° C. for 2 hrs under N₂. On completion, the reaction was diluted with EA (80 mL). The organic layer was washed with water (80 mL×2), dried over Na₂SO₄ and concentrated in vacuo. The residue was purified by flash silica gel chromatography (ISCO®; 80 g SepaFlash® Silica Flash Column, Eluent of 0˜10% Ethyl acetate/Petroleum ether gradient @ 100 mL/min) to give the title compound (2.92 g, 65% yield) as yellow solid. ¹H NMR (400 MHz, DMSO-d₆) δ 7.96 (d, J=1.2 Hz, 1H), 7.91 (d, J=8.0 Hz, 1H), 7.62 (d, J=8.4 Hz, 1H), 7.49-7.45 (m, 2H), 7.41-7.28 (m, 8H), 7.12 (dd, J=1.6, 8.4 Hz, 1H), 6.60 (d, J=8.0 Hz, 1H), 5.44 (d, J=8.0 Hz, 4H), 4.05 (s, 3H); LC-MS (ESI⁺) m/z 501.8 (M+H)⁺.

### Step 3—3-(2,6-Dibenzyloxy-3-pyridyl)-6-[4-(dimethoxymethyl)-1-piperidyl]-1-methyl-indazole

To a solution of 6-bromo-3-(2,6-dibenzyloxy-3-pyridyl)-1-methyl-indazole (1 g, 2.00 mmol), 4-(dimethoxymethyl)piperidine (477 mg, 3.00 mmol, CAS #188646-83-5) and XantPhos Pd G3 (189 mg, 199 μmol) in dioxane (15 mL) was added Cs₂CO₃ (1.95 g, 6.00 mmol). The reaction was then stirred at 110° C. for 16 hrs under N₂. On completion, the reaction was diluted with EA (50 mL). The organic layer was washed with water (50 mL×2), dried over Na₂SO₄ and concentrated in vacuo. The residue was purified by flash silica gel chromatography (ISCO®; 20 g SepaFlash® Silica Flash Column, Eluent of 0˜30% Ethyl acetate/Petroleum ether gradient @ 80 mL/min) to give the title compound (835 mg, 72% yield) as yellow oil. ¹H NMR (400 MHz, DMSO-d₆) δ 7.88 (d, J=8.0 Hz, 1H), 7.49-7.28 (m, 11H), 6.84-6.76 (m, 2H), 6.56 (d, J=8.0 Hz, 1H), 5.43 (d, J=13.2 Hz, 4H), 4.10 (d, J=6.4 Hz, 1H), 3.95 (s, 3H), 3.79 (d, J=12.4 Hz, 2H), 3.28 (s, 6H), 2.72-2.65 (m, 2H), 1.80-1.70 (m, 3H), 1.44-1.31 (m, 2H); LC-MS (ESI⁺) m/z 579.3 (M+H)⁺.

### Step 4—3-[6-[4-(Dimethoxymethyl)-1-piperidyl]-1-methyl-indazol-3-yl]piperidine-2,6-dione

To a solution of 3-(2,6-dibenzyloxy-3-pyridyl)-6-[4-(dimethoxymethyl)-1-piperidyl]-1-methyl-indazole (400 mg, 691 mol) in THF (20 mL) was added Pd/C (300 mg, 281 μmol, 10 wt %) under H₂. The suspension was degassed under vacuum and purged with H₂ several times. The mixture was then stirred under H₂ (15 psi) at 25° C. for 16 hrs. On completion, the reaction mixture was filtered and the filtrated was concentrated in vacuo to give the title compound (250 mg, 90% yield) as yellow oil. ¹H NMR (400 MHz, DMSO-d₆) δ 10.84 (s, 1H), 7.47 (d, J=9.2 Hz, 1H), 6.89 (dd, J=1.6, 8.8 Hz, 1H), 6.82 (s, 1H), 4.29-4.21 (m, 2H), 4.10 (d, J=6.8 Hz, 1H), 3.88 (s, 3H), 3.83-3.77 (m, 2H), 3.28 (s, 6H), 3.25-3.23 (m, 1H), 2.72-2.65 (m, 2H), 2.42 (t, J=8.0 Hz, 1H), 2.20-2.08 (m, 2H), 1.73 (d, J=10.4 Hz, 2H), 1.43-1.31 (m, 2H); LC-MS (ESI⁺) m/z 401.0 (M+H)⁺.

### Step 5—1-[3-(2,6-Dioxo-3-piperidyl)-1-methyl-indazol-6-yl]piperidine-4-carbaldehyde

A mixture of 3-[6-[4-(dimethoxymethyl)-1-piperidyl]-1-methyl-indazol-3-yl]piperidine-2,6-dione (60 mg, 149 μmol) in HCOOH (1.5 mL) was stirred at 70° C. for 1 hr. On completion, the reaction was concentrated in vacuo to give the title compound (53 mg, 99% yield) as brown oil. LC-MS (ESI⁺) m z 373.0 (M+18+H)⁺.

### Example 2. Preparation of Compounds of the Invention (Method 1)

### Synthesis of N-[(1R)-2-[4-[[4-[1-(2,6-dioxo-3-piperidyl)-3-methyl-2-oxo-benzimidazol-4-yl]piperazin-1-yl]methyl]cyclohexoxy]-1-methyl-ethyl]-4-[[4-[(3S)-3-hydroxy-3-methyl-1-piperidyl]-5-(trifluoromethyl)pyrimidin-2-yl]amino]-3-methyl-benzenesulfonamide (I-766)

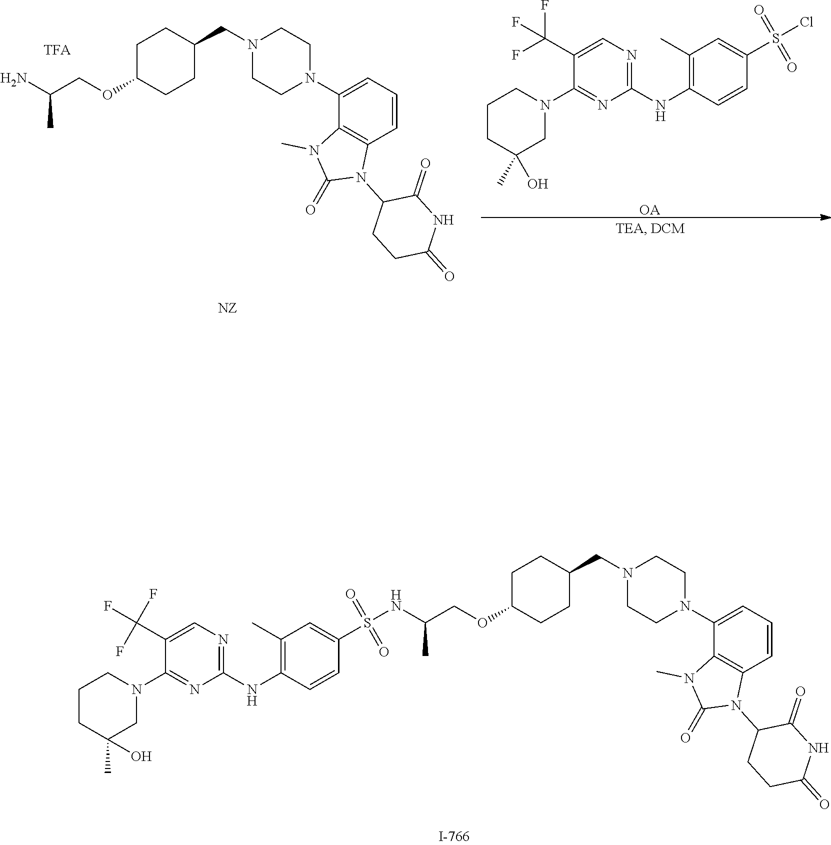 [SMILES: C[C@@H](N)CO[C@H]1CC[C@H](CN2CCN(c3cccc4c3n(C)c(=O)n4C3CCC(=O)NC3=O)CC2)CC1 ; Cc1cc(S(=O)(=O)Cl)ccc1Nc1ncc(C(F)(F)F)c(N2CCC[C@](C)(O)C2)n1 ; "OA" ; "TEA, DCM" ; Cc1cc(S(=O)(=O)N[C@H](C)CO[C@H]2CC[C@H](CN3CCN(c4cccc5c4n(C)c(=O)n5C4CCC(=O)NC4=O)CC3)CC2)ccc1Nc1ncc(C(F)(F)F)c(N2CCC[C@](C)(O)C2)n1]

To a solution of 3-[4-[4-[[4-[(2R)-2-aminopropoxy]cyclohexyl]methyl]piperazin-1-yl]-3-methyl-2-oxo-benzimidazol-1-yl]piperidine-2,6-dione (55.0 mg, 87.7 μmol, Intermediate NZ) in DCM (1 mL) was added TEA (8.88 mg, 87.7 mol) until the pH=8-10. Then 4-[[4-[(3S)-3-hydroxy-3-methyl-1-piperidyl]-5-(trifluoromethyl)pyrimidin-2-yl]amino]-3-methyl-benzenesulfonyl chloride (33.0 mg, 70.9 μmol, Intermediate OA) was added and the mixture was stirred at 25° C. for 0.5 hr. On completion, the mixture was concentrated in vacuo to give the residue. The residue was purified by prep-HPLC (column: Waters xbridge 150*25 mm 10 um; mobile phase: [water(NH₄HCO₃)-ACN]; gradient:42%-72% B over 18 min) to give the title compound (11.3 mg, 13% yield) as white solid. ¹H NMR (400 MHz, DMSO-d₆) δ 11.09 (s, 1H), 9.09 (s, 1H), 8.34 (s, 1H), 7.88 (d, J=8.8 Hz, 1H), 7.64 (s, 1H), 7.62-7.58 (m, 1H), 7.47 (d, J=7.6 Hz, 1H), 7.00-6.95 (m, 1H), 6.92 (s, 1H), 6.90-6.86 (m, 1H), 5.35 (dd, J=5.2, 12.4 Hz, 1H), 4.47 (s, 1H), 3.60 (s, 3H), 3.41 (d, J=12.8 Hz, 1H), 3.26 (d, J=12.4 Hz, 3H), 3.15-3.10 (m, 1H), 3.01-2.78 (m, 7H), 2.71-2.62 (m, 4H), 2.33 (s, 3H), 2.08 (d, J=6.8 Hz, 2H), 2.02-1.93 (m, 2H), 1.87-1.78 (m, 3H), 1.78-1.69 (m, 3H), 1.56 (d, J=2.8 Hz, 2H), 1.50-1.31 (m, 3H), 1.23 (s, 1H), 1.04 (s, 3H), 0.99 (d, J=14.4 Hz, 2H), 0.92 (d, J=6.4 Hz, 3H), 0.80-0.76 (m, 1H); LC-MS (ESI⁺) m/z 941.3 (M+H)⁺.

TABLE 2
Compounds synthesized via Method 1, with the coupling
of the corresponding amines and sulfonyl chlorides.

| | | | LCMS | |
|---|---|---|---|---|
| | | | (ESI+) | |
| | | Sulfonyl | m/z | |
| I-# | Amine | Chloride | (M + H)⁺ | ¹H NMR (400 MHz, DMSO-d₆) δ |
| I-515 | NG | ND | 943.5 | 11.11 (s, 1H), 9.64 (s, 1H), 8.60 (s, 1H), 7.79 (d, J = 8.4 |
| | | | | Hz, 1H), 7.66 (d, J = 1.6 Hz, 1H), 7.61-7.54 (m, 2H), 6.99- |
| | | | | 6.89 (m, 2H), 5.36 (dd, J = 5.2, 12.8 Hz, 1H), 4.01 (s, 1H), |
| | | | | 3.48 (d, J = 1.6 Hz, 3H), 3.37 (t, J = 5.6 Hz, 2H), 3.05 (d, |
| | | | | J = 4.0 Hz, 1H), 2.91-2.86 (m, 4H), 2.79-2.71 (m, 2H), |
| | | | | 2.68-2.59 (m, 2H), 2.33 (s, 3H), 2.15-1.97 (m, 6H), 1.96- |
| | | | | 1.87 (m, 4H), 1.74 (d, J = 12.4 Hz, 2H), 1.70-1.61 (m, |
| | | | | 6H), 1.46 (d, J = 11.2 Hz, 2H), 1.38 (dd, J = 3.2, 13.2 Hz, |
| | | | | 3H), 1.13 (s, 3H), 1.04 (d, J = 12.8 Hz, 2H), 0.86-0.76 (m, |
| | | | | 2H) |
| I-517 | DQ | ND | 925.7 | 11.08 (s, 1H), 9.65 (s, 1H), 8.60 (s, 1H), 7.79 (d, J = 8.4 |
| | | | | Hz, 1H), 7.66 (d, J = 2.0 Hz, 1H), 7.61-7.55 (m, 2H), 7.09 |
| | | | | (d, J = 0.8 Hz, 1H), 6.99 (d, J = 8.0 Hz, 1H), 6.92-6.88 |
| | | | | (m, 1H), 5.33 (dd, J = 5.2, 12.8 Hz, 1H), 4.10-3.94 (m, |
| | | | | 1H), 3.33 (s, 3H), 3.10-2.99 (m, 2H), 2.92-2.85 (m, 5H), |
| | | | | 2.74-2.67 (m, 2H), 2.66-2.55 (m, 2H), 2.33 (s, 3H), 2.13- |
| | | | | 1.88 (m, 10H), 1.79-1.60 (m, 9H), 1.46 (br d, J = 11.6 |
| | | | | Hz, 2H), 1.39-1.33 (m, 2H), 1.13 (s, 3H), 1.09-1.00 (m, |
| | | | | 2H), 0.87-0.74 (m, 2H) |
| I-664 | RH | OA | 956.2 | 11.10 (s, 1H), 9.10 (s, 1H), 8.35 (s, 1H), 7.92-7.80 (m, |
| | | | | 2H), 7.63-7.50 (m, 2H), 6.98-6.77 (m, 2H), 5.34 (dd, J = |
| | | | | 5.6, 12.4 Hz, 1H), 4.54-4.36 (m, 1H), 3.65-3.56 (m, 5H), |
| | | | | 3.44-3.37 (m, 2H), 3.29-3.20 (m, 3H), 3.13-2.98 (m, |
| | | | | 4H), 2.94-2.82 (m, 1H), 2.75-2.58 (m, 4H), 2.37 (d, J = |
| | | | | 2.0 Hz, 2H), 2.32 (s, 3H), 2.18 (d, J = 3.6 Hz, 4H), 2.05- |
| | | | | 1.95 (m, 3H), 1.90-1.66 (m, 6H), 1.64-1.52 (m, 3H), 1.48- |
| | | | | 1.38 (m, 1H), 1.31-1.18 (m, 2H), 1.04 (s, 3H) |
| I-666 | OK | OA | 970.4 | 11.10 (s, 1H), 9.10 (s, 1H), 8.34 (s, 1H), 7.84 (d, J = 8.8 |
| | | | | Hz, 1H), 7.72 (s, 1H), 7.67-7.55 (m, 2H), 6.90-6.80 (m, |
| | | | | 1H), 6.77-6.66 (m, 1H), 5.44-5.24 (m, 1H), 4.50-4.41 |
| | | | | (m, 1H), 3.69-3.55 (m, 1H), 3.47 (s, 3H), 3.44-3.37 (m, |
| | | | | 2H), 3.28-3.16 (m, 5H), 2.95-2.83 (m, 1H), 2.74-2.57 |
| | | | | (m, 5H), 2.33 (s, 3H), 2.31-2.23 (m, 4H), 2.18-2.09 (m, |
| | | | | 4H), 2.05-1.94 (m, 2H), 1.89-1.65 (m, 4H), 1.64-1.50 |
| | | | | (m, 5H), 1.49-1.38 (m, 1H), 1.32-1.23 (m, 2H), 1.21 (s, |
| | | | | 3H), 1.07-1.00 (m, 3H) |
| I-686 | RF | OA | 938.3 | 11.08 (s, 1H), 9.10 (s, 1H), 8.34 (s, 1H), 7.83 (d, J = 8.4 |
| | | | | Hz, 1H), 7.66-7.49 (m, 3H), 6.99-6.89 (m, 1H), 6.71 (d, |
| | | | | J = 8.0 Hz, 1H), 6.62 (d, J = 8.0 Hz, 1H), 5.35-5.25 (m, |
| | | | | 1H), 4.46 (d, J = 2.0 Hz, 1H), 3.90 (t, J = 6.8 Hz, 2H), 3.66- |
| | | | | 3.56 (m, 2H), 3.54 (s, 3H), 3.47-3.44 (m, 3H), 3.39- |
| | | | | 3.38 (m, 2H), 3.26-3.24 (m, 2H), 2.94-2.74 (m, 4H), 2.71- |
| | | | | 2.57 (m, 3H), 2.53 (s, 1H), 2.42 (s, 4H), 2.32 (s, 3H), 2.15- |
| | | | | 2.07 (m, 1H), 2.00-1.93 (m, 1H), 1.81-1.73 (m, 1H), |
| | | | | 1.67 (d, J = 7.2 Hz, 4H), 1.59-1.50 (m, 2H), 1.47-1.37 |
| | | | | (m, 1H), 1.18-1.07 (m, 4H), 1.03 (s, 3H) |
| I-717 | SR | OA | 917.2 | 10.89 (s, 1H), 9.10 (s, 1H), 8.34 (s, 1H), 7.87-7.82 (m, |
| | | | | 2H), 7.60 (d, J = 1.6 Hz, 1H), 7.54 (dd, J = 2.0, 8.4 Hz, |
| | | | | 1H), 6.88 (d, J = 2.4 Hz, 1H), 6.81-6.77 (m, 1H), 6.76- |
| | | | | 6.71 (m, 1H), 5.07 (d, J = 6.4 Hz, 1H), 4.46 (s, 1H), 4.34- |
| | | | | 4.28 (m, 1H), 3.63-3.56 (m, 2H), 3.41-3.38 (m, 4H), 3.27- |
| | | | | 3.23 (m, 2H), 2.83-2.73 (m, 1H), 2.69-2.62 (m, 1H), |
| | | | | 2.56-2.55 (m, 2H), 2.45 (s, 2H), 2.32 (s, 3H), 2.20-2.09 |
| | | | | (m, 6H), 2.01-1.93 (m, 4H), 1.86-1.70 (m, 6H), 1.55- |
| | | | | 1.52 (m, 4H), 1.48-1.38 (m, 1H), 1.21-1.12 (m, 2H), 1.03 |
| | | | | (s, 3H) |
| I-745 | PI | OA | 847.4 | 10.82 (s, 1H), 9.08 (s, 1H), 8.34 (s, 1H), 7.82 (d, J = 8.4 |
| | | | | Hz, 1H), 7.63 (s, 1H), 7.59 (d, J = 8.4 Hz, 1H), 7.51 (d, J = |
| | | | | 7.2 Hz, 1H), 7.10 (d, J = 8.8 Hz, 1H), 6.91 (d, J = 2.0 Hz, |
| | | | | 1H), 6.86 (dd, J = 2.0, 8.8 Hz, 1H), 4.44 (s, 1H), 4.03 (dd, |
| | | | | J = 4.8, 12.4 Hz, 1H), 3.65-3.55 (m, 1H), 3.44-3.37 (m, |
| | | | | 1H), 3.28-3.22 (m, 3H), 3.11 (s, 4H), 2.93-2.81 (m, 1H), |
| | | | | 2.79-2.66 (m, 1H), 2.39 (s, 3H), 2.32 (s, 3H), 2.28-2.14 |
| | | | | (m, 2H), 2.04-2.03 (m, 2H), 1.97-1.89 (m, 1H), 1.80- |
| | | | | 1.73 (m, 1H), 1.69-1.63 (m, 4H), 1.58-1.50 (m, 2H), 1.45- |
| | | | | 1.33 (m, 2H), 1.19-1.07 (m, 2H), 1.03 (s, 3H), 0.83- |
| | | | | 0.71 (m, 2H) |
| I-765 | NW | ND | 958.3 | 11.10 (s, 1H), 9.62 (s, 1H), 8.60 (s, 1H), 7.81 (d, J = 8.4 |
| | | | | Hz, 1H), 7.67 (d, J = 1.6 Hz, 1H), 7.61 (dd, J = 2.0, 8.4 Hz, |
| | | | | 1H), 7.50 (d, J = 7.2 Hz, 1H), 6.85 (d, J = 8.8 Hz, 1H), 6.75- |
| | | | | 6.65 (m, 1H), 5.33 (dd, J = 5.4, 12.8 Hz, 1H), 4.00 (s, 1H), |
| | | | | 3.46 (d, J = 1.6 Hz, 3H), 3.29-3.18 (m, 3H), 3.17-3.10 |
| | | | | (m, 1H), 3.06-2.98 (m, 1H), 2.90 (d, J = 11.2 Hz, 4H), |
| | | | | 2.75-2.57 (m, 4H), 2.44 (s, 3H), 2.33 (s, 3H), 2.17-1.97 |
| | | | | (m, 5H), 1.83 (d, J = 2.4 Hz, 2H), 1.72 (d, J = 11.6 Hz, 2H), |
| | | | | 1.63 (d, J = 12.4 Hz, 2H), 1.46 (d, J = 11.2 Hz, 2H), 1.40- |
| | | | | 1.30 (m, 3H), 1.13 (s, 3H), 1.00 (d, J = 11.6 Hz, 2H), 0.95 |
| | | | | (d, J = 6.4 Hz, 3H), 0.86-0.73 (m, 2H) |
| I-766 | NZ | OA | 941.3 | 11.09 (s, 1H), 9.09 (s, 1H), 8.34 (s, 1H), 7.88 (d, J = 8.8 |
| | | | | Hz, 1H), 7.64 (s, 1H), 7.62-7.58 (m, 1H), 7.47 (d, J = 7.6 |
| | | | | Hz, 1H), 7.00-6.95 (m, 1H), 6.92 (s, 1H), 6.90-6.86 (m, |
| | | | | 1H), 5.35 (dd, J = 5.2, 12.4 Hz, 1H), 4.47 (s, 1H), 3.60 (s, |
| | | | | 3H), 3.41 (d, J = 12.8 Hz, 1H), 3.26 (d, J = 12.4 Hz, 3H), |
| | | | | 3.15-3.10 (m, 1H), 3.01-2.78 (m, 7H), 2.71-2.62 (m, |
| | | | | 4H), 2.33 (s, 3H), 2.08 (d, J = 6.8 Hz, 2H), 2.02-1.93 (m, |
| | | | | 2H), 1.87-1.78 (m, 3H), 1.78-1.69 (m, 3H), 1.56 (d, J = |
| | | | | 2.8 Hz, 2H), 1.50-1.31 (m, 3H), 1.23 (s, 1H), 1.04 (s, 3H), |
| | | | | 0.99 (d, J = 14.4 Hz, 2H), 0.92 (d, J = 6.4 Hz, 3H), 0.80- |
| | | | | 0.76 (m, 1H) |
| I-767 | OE | OA | 960.4 | 11.15-11.05 (m, 1H), 9.08 (s, 1H), 8.34 (s, 1H), 7.88 (d, J = |
| | | | | 8.4 Hz, 1H), 7.66-7.58 (m, 2H), 7.49-7.42 (m, 1H), |
| | | | | 6.85 (d, J = 8.4 Hz, 1H), 6.75-6.67 (m, 1H), 5.33 (dd, J = |
| | | | | 5.6, 13.2 Hz, 1H), 4.46 (s, 1H), 3.63-3.54 (m, 1H), 3.46 |
| | | | | (s, 3H), 3.41 (d, J = 13.2 Hz, 1H), 3.29-3.22 (m, 3H), 3.11 |
| | | | | (d, J = 9.6 Hz, 1H), 2.98 (s, 1H), 2.92 (s, 4H), 2.88-2.83 |
| | | | | (m, 1H), 2.74-2.62 (m, 2H), 2.47-2.38 (m, 4H), 2.33 (s, |
| | | | | 3H), 2.06 (d, J = 7.2 Hz, 2H), 2.03-1.96 (m, 1H), 1.85- |
| | | | | 1.69 (m, 5H), 1.61-1.51 (m, 2H), 1.49-1.34 (m, 2H), 1.04 |
| | | | | (s, 3H), 0.99 (d, J = 13.6 Hz, 2H), 0.92 (s, 3H), 0.84-0.71 |
| | | | | (m, 2H) |
| I-768 | OI | OA | 970.4 | 11.03 (s, 1H), 9.09 (s, 1H), 8.34 (s, 1H), 7.84 (d, J = 8.4 |
| | | | | Hz, 1H), 7.69 (s, 1H), 7.62 (s, 1H), 7.58 (d, J = 8.8 Hz, 1H), |
| | | | | 6.83 (d, J = 9.2 Hz, 1H), 6.71 (t, J = 8.0 Hz, 1H), 5.36- |
| | | | | 5.28 (m, 1H), 4.46 (s, 1H), 3.63-3.55 (m, 1H), 3.46 (s, |
| | | | | 3H), 3.23 (s, 2H), 3.22-3.21 (m, 2H), 3.19-3.18 (m, 2H), |
| | | | | 2.94-2.82 (m, 2H), 2.67 (s, 2H), 2.63-2.62 (m, 2H), 2.59- |
| | | | | 2.58 (m, 2H), 2.33 (s, 3H), 2.18-2.08 (m, 5H), 2.03- |
| | | | | 1.95 (m, 2H), 1.93-1.89 (m, 2H), 1.87-1.85 (m, 2H), 1.78- |
| | | | | 1.70 (m, 3H), 1.59-1.51 (m, 3H), 1.47-1.41 (m, 1H), |
| | | | | 1.23 (s, 6H), 1.04 (s, 3H) |
| I-769^{b} | OM | ON | 898.4 | 10.75 (s, 1H), 8.82 (s, 1H), 7.98 (dd, J = 6.8, 8.6 Hz, 1H), |
| | | | | 7.93 (d, J = 1.4 Hz, 1H), 7.63 (s, 1H), 7.58 (d, J = 8.6 Hz, |
| | | | | 1H), 7.44 (d, J = 7.2 Hz, 1H), 6.80 (d, J = 8.2 Hz, 1H), 6.50 |
| | | | | (s, 1H), 6.45 (dd, J = 2.4, 8.2 Hz, 1H), 5.45 (d, J = 7.1 Hz, |
| | | | | 1H), 4.71-4.65 (m, 1H), 4.56 (d, J = 2.4 Hz, 1H), 4.23- |
| | | | | 4.18 (m, 1H), 4.07 (s, 1H), 2.98-2.92 (m, 1H), 2.67-2.54 |
| | | | | (m, 6H), 2.35 (s, 4H), 2.33-2.32 (m, 2H), 2.33-2.32 (m, |
| | | | | 1H), 2.12 (s, 3H), 2.02 (d, J = 7.2 Hz, 2H), 1.84-1.77 (m, |
| | | | | 4H), 1.69-1.61 (m, 9H), 1.49 (d, J = 3.2 Hz, 3H), 1.37- |
| | | | | 1.28 (m, 3H), 1.15 (s, 1H), 0.94 (d, J = 6.4 Hz, 6H), 0.80- |
| | | | | 0.71 (m, 3H) |
| I-770^{b} | PK | ON | 901.5 | 10.81 (s, 1H), 8.82 (s, 1H), 7.97 (d, J = 8.8 Hz, 1H), 7.93 |
| | | | | (s, 1H), 7.62 (s, 1H), 7.60-7.55 (m, 1H), 7.44 (d, J = 7.2 |
| | | | | Hz, 1H), 6.90 (dd, J = 1.6, 13.2 Hz, 1H), 6.84-6.79 (m, |
| | | | | 1H), 6.78-6.71 (m, 1H), 5.38 (dd, J = 2.0, 8.0 Hz, 1H), |
| | | | | 4.73-4.63 (m, 1H), 4.56 (s, 1H), 4.40-4.31 (m, 1H), 4.08 |
| | | | | (s, 1H), 3.16-3.09 (m, 2H), 2.99-2.89 (m, 2H), 2.82 (d, J = |
| | | | | 10.0 Hz, 2H), 2.45-2.41 (m, 1H), 2.35 (s, 3H), 2.25- |
| | | | | 2.13 (m, 2H), 2.12-2.04 (m, 2H), 2.02-1.99 (m, 2H), 1.92- |
| | | | | 1.85 (m, 2H), 1.85-1.75 (m, 4H), 1.65 (dd, J = 4.4, 8.4 |
| | | | | Hz, 10H), 1.56-1.46 (m, 6H), 1.39-1.29 (m, 2H), 0.98 (s, |
| | | | | 1H), 0.94 (d, J = 6.4 Hz, 3H), 0.81-0.70 (m, 2H) |
| I-771 | OO | OA | 843.2 | 10.78 (s, 1H), 9.09 (s, 1H), 8.34 (s, 1H), 7.87-7.78 (m, |
| | | | | 1H), 7.67-7.50 (m, 3H), 6.82-6.76 (m, 2H), 6.72-6.63 |
| | | | | (m, 1H), 4.45 (s, 1H), 3.76 (d, J = 5.2 Hz, 1H), 3.73 (s, 3H), |
| | | | | 3.60 (dt, J = 2.0, 6.4 Hz, 1H), 3.40 (d, J = 12.8 Hz, 1H), |
| | | | | 3.30 (s, 3H), 3.27-3.22 (m, 2H), 2.90 (s, 4H), 2.69-2.64 |
| | | | | (m, 1H), 2.61 (dd, J = 4.8, 12.0 Hz, 1H), 2.41 (s, 2H), 2.32 |
| | | | | (s, 3H), 2.22-2.16 (m, 2H), 2.05-1.99 (m, 3H), 1.80- |
| | | | | 1.74 (m, 1H), 1.69-1.63 (m, 3H), 1.58-1.52 (m, 2H), 1.44- |
| | | | | 1.33 (m, 2H), 1.19-1.08 (m, 2H), 1.03 (s, 3H), 0.84- |
| | | | | 0.72 (m, 2H) |
| I-772 | OQ | OA | 831.2 | 10.82 (s, 1H), 9.10 (s, 1H), 8.34 (s, 1H), 7.83 (d, J = 8.4 |
| | | | | Hz, 1H), 7.63 (s, 1H), 7.60-7.57 (m, 1H), 7.53 (d, J = 7.6 |
| | | | | Hz, 1H), 7.01 (d, J = 14.0 Hz, 1H), 6.97-6.90 (m, 2H), |
| | | | | 4.46 (s, 1H), 3.80-3.76 (m, 1H), 3.64-3.56 (m, 2H), 3.26- |
| | | | | 3.23 (m, 2H), 2.99-2.81 (m, 5H), 2.69-2.60 (m, 1H), |
| | | | | 2.48-2.38 (m, 4H), 2.32 (s, 3H), 2.20-2.16 (m, 1H), 2.07- |
| | | | | 2.04 (m, 2H), 2.01-1.95 (m, 1H), 1.80-1.73 (m, 1H), |
| | | | | 1.69-1.62 (m, 4H), 1.58-1.49 (m, 2H), 1.45-1.30 (m, |
| | | | | 2H), 1.23 (s, 1H), 1.18-1.07 (m, 2H), 1.03 (s, 3H), 0.84- |
| | | | | 0.71 (m, 2H) |
| I-773^{b} | PI | ON | 845.3 | 10.83 (s, 1H), 8.85 (s, 1H), 7.95-7.88 (m, 2H), 7.62 (s, |
| | | | | 1H), 7.57 (d, J = 8.8 Hz, 1H), 7.52 (d, J = 7.2 Hz, 1H), 7.35- |
| | | | | 7.29 (m, 1H), 7.24 (d, J = 7.6 Hz, 1H), 7.10 (d, J = 8.8 |
| | | | | Hz, 1H), 6.91 (d, J = 2.4 Hz, 1H), 6.89-6.83 (m, 1H), 4.73- |
| | | | | 4.61 (m, 1H), 4.57 (s, 1H), 4.24 (d, J = 5.6 Hz, 1H), 4.10- |
| | | | | 3.99 (m, 2H), 3.10 (s, 4H), 2.92-2.82 (m, 1H), 2.39 (s, |
| | | | | 4H), 2.34 (s, 3H), 2.24-2.14 (m, 2H), 2.04 (d, J = 7.2 Hz, |
| | | | | 2H), 1.97-1.89 (m, 1H), 1.86 (s, 1H), 1.72-1.61 (m, 9H), |
| | | | | 1.50 (d, J = 3.2 Hz, 3H), 1.38-1.22 (m, 2H), 1.21-1.08 |
| | | | | (m, 2H), 0.84-0.71 (m, 2H) |
| I-774 | OR | OA | 875.2 | 10.82 (s, 1H), 9.11 (s, 1H), 8.34 (s, 1H), 7.81 (d, J = 8.4 |
| | | | | Hz, 1H), 7.70-7.55 (m, 3H), 7.09 (d, J = 8.8 Hz, 1H), 6.92- |
| | | | | 6.80 (m, 2H), 4.45 (s, 1H), 4.03 (dd, J = 5.2, 12.0 Hz, 1H), |
| | | | | 3.63-3.53 (m, 3H), 3.44-3.38 (m, 4H), 3.26-3.20 (m, |
| | | | | 2H), 3.10-3.05 (m, 4H), 2.83-2.63 (m, 2H), 2.32 (s, 3H), |
| | | | | 2.26-2.20 (m, 1H), 2.18-2.12 (s, 2H), 1.96-1.89 (m, |
| | | | | 1H), 1.81-1.71 (m, 5H), 1.69-1.37 (m, 8H), 1.03 (s, 3H) |
| I-775 | PL | OA | 828.2 | 10.75 (s, 1H), 9.09 (s, 1H), 8.34 (s, 1H), 7.83 (d, J = 8.4 |
| | | | | Hz, 1H), 7.64 (d, J = 1.6 Hz, 1H), 7.59 (dd, J = 2.0, 8.4 Hz, |
| | | | | 1H), 7.51 (d, J = 7.2 Hz, 1H), 6.89 (t, J = 8.0 Hz, 1H), 6.23 |
| | | | | (s, 1H), 6.18-6.09 (m, 2H), 5.60 (d, J = 7.6 Hz, 1H), 4.45 |
| | | | | (s, 1H), 4.35-4.24 (m, 1H), 3.65-3.55 (m, 1H), 3.39 (s, |
| | | | | 2H), 3.26 (s, 2H), 3.01 (s, 4H), 2.91-2.82 (m, 1H), 2.77- |
| | | | | 2.66 (m, 1H), 2.38 (s, 4H), 2.32 (s, 3H), 2.08 (td, J = 4.4, |
| | | | | 8.8 Hz, 1H), 2.03 (d, J = 7.2 Hz, 2H), 1.90-1.73 (m, 2H), |
| | | | | 1.66 (t, J = 13.2 Hz, 4H), 1.59-1.50 (m, 2H), 1.47-1.30 |
| | | | | (m, 2H), 1.20-1.07 (m, 2H), 1.03 (s, 3H), 0.83-0.70 (m, |
| | | | | 2H) |
| I-776 | PN | OA | 849.3 | 10.80 (s, 1H), 9.14-9.04 (m, 1H), 8.35 (s, 1H), 7.88-7.80 |
| | | | | (m, 2H), 7.68 (d, J = 1.6 Hz, 1H), 7.65-7.60 (m, 1H), 7.02- |
| | | | | 6.90 (m, 3H), 4.57-4.39 (m, 2H), 3.82-3.75 (m, 1H), |
| | | | | 3.65-3.56 (m, 1H), 3.43-3.37 (m, 1H), 3.30-3.27 (m, |
| | | | | 2H), 3.26-3.20 (m, 2H), 3.02-2.93 (m, 1H), 2.70-2.55 |
| | | | | (m, 5H), 2.47-2.44 (m, 1H), 2.32 (s, 3H), 2.22-2.07 (m, |
| | | | | 4H), 2.02-1.88 (m, 2H), 1.80-1.63 (m, 4H), 1.59-1.50 |
| | | | | (m, 3H), 1.47-1.37 (m, 1H), 1.30-1.16 (m, 3H), 1.04 (s, |
| | | | | 3H) |
| I-777 | PP | OA | 831.1 | 10.80 (s, 1H), 9.08 (s, 1H), 8.34 (s, 1H), 7.88-7.77 (m, |
| | | | | 1H), 7.64 (s, 1H), 7.61-7.56 (m, 1H), 7.51 (d, J = 7.2 Hz, |
| | | | | 1H), 7.07 (t, J = 8.8 Hz, 1H), 6.74-6.64 (m, 2H), 4.44 (s, |
| | | | | 1H), 3.87 (dd, J = 4.8, 12.2 Hz, 1H), 3.66-3.53 (m, 1H), |
| | | | | 3.40 (d, J = 13.2 Hz, 2H), 3.27-3.22 (m, 2H), 3.10 (s, 4H), |
| | | | | 2.95-2.79 (m, 1H), 2.75-2.66 (m, 1H), 2.39 (s, 4H), 2.32 |
| | | | | (s, 3H), 2.17-2.01 (m, 3H), 1.99-1.88 (m, 1H), 1.79- |
| | | | | 1.61 (m, 5H), 1.59-1.49 (m, 2H), 1.46-1.29 (m, 2H), 1.19- |
| | | | | 1.07 (m, 2H), 1.03 (s, 3H), 0.86-0.69 (m, 2H) |
| I-778 | PR | OA | 889.4 | 10.82 (s, 1H), 9.11 (s, 1H), 8.34 (s, 1H), 7.81 (d, J = 8.4 |
| | | | | Hz, 1H), 7.68-7.58 (m, 3H), 7.09 (d, J = 8.4 Hz, 1H), 6.91- |
| | | | | 6.79 (m, 2H), 4.44 (s, 1H), 4.06-3.99 (m, 1H), 3.64- |
| | | | | 3.54 (m, 3H), 3.44-3.36 (m, 2H), 3.25-3.20 (m, 2H), 3.06- |
| | | | | 2.99 (m, 1H), 2.87-2.79 (m, 1H), 2.77-2.66 (m, 2H), |
| | | | | 2.59-2.54 (m, 2H), 2.43 (d, J = 14.4 Hz, 2H), 2.32 (s, 3H), |
| | | | | 2.29-2.16 (m, 2H), 1.99 (d, J = 14.4 Hz, 1H), 1.95-1.86 |
| | | | | (m, 2H), 1.75 (t, J = 8.4 Hz, 3H), 1.67-1.60 (m, 3H), 1.57- |
| | | | | 1.41 (m, 5H), 1.03 (s, 3H), 0.95 (d, J = 6.0 Hz, 3H) |
| I-779 | PT | OA | 889.3 | 10.82 (s, 1H), 9.11 (s, 1H), 8.34 (s, 1H), 7.81 (d, J = 8.4 |
| | | | | Hz, 1H), 7.67-7.58 (m, 3H), 7.09 (d, J = 8.4 Hz, 1H), 6.91- |
| | | | | 6.80 (m, 2H), 4.44 (s, 1H), 4.03 (dd, J = 4.8, 12.0 Hz, 1H), |
| | | | | 3.63-3.54 (m, 3H), 3.43-3.39 (m, 2H), 3.25-3.21 (m, |
| | | | | 2H), 3.07-2.99 (m, 1H), 2.86-2.79 (m, 1H), 2.75-2.70 |
| | | | | (m, 1H), 2.57 (s, 2H), 2.43 (d, J = 14.4 Hz, 2H), 2.32 (s, |
| | | | | 3H), 2.27 (d, J = 9.2 Hz, 1H), 2.21 (dd, J = 4.0, 12.4 Hz, |
| | | | | 1H), 1.99 (d, J = 14.4 Hz, 1H), 1.94-1.88 (m, 2H), 1.75 (t, |
| | | | | J = 9.2 Hz, 4H), 1.66-1.59 (m, 3H), 1.56-1.48 (m, 3H), |
| | | | | 1.46-1.37 (m, 2H), 1.03 (s, 3H), 0.95 (d, J = 6.0 Hz, 3H) |
| I-780^{b} | OQ | ON | 829.4 | 10.86-10.74 (m, 1H), 8.83 (s, 1H), 7.95-7.87 (m, 2H), |
| | | | | 7.62 (s, 1H), 7.57 (dd, J = 2.4, 8.8 Hz, 1H), 7.53-7.47 (m, |
| | | | | 1H), 7.00 (d, J = 14.0 Hz, 1H), 6.96-6.91 (m, 2H), 4.73- |
| | | | | 4.61 (m, 1H), 4.55 (d, J = 2.4 Hz, 1H), 4.07 (d, J = 2.0 Hz, |
| | | | | 1H), 3.79 (dd, J = 4.8, 11.6 Hz, 1H), 2.96 (s, 4H), 2.91- |
| | | | | 2.82 (m, 1H), 2.43 (s, 6H), 2.34 (s, 3H), 2.26-2.11 (m, |
| | | | | 3H), 2.05 (d, J = 7.2 Hz, 2H), 2.01-1.92 (m, 1H), 1.74- |
| | | | | 1.59 (m, 10H), 1.55-1.48 (m, 3H), 1.37-1.23 (m, 2H), |
| | | | | 1.20-1.08 (m, 2H), 0.85-0.71 (m, 2H) |
| I-781^{b} | PL | ON | 826.4 | 10.74 (s, 1H), 8.88-8.79 (m, 1H), 7.93-7.85 (m, 2H), 7.62 |
| | | | | (s, 1H), 7.60-7.55 (m, 1H), 7.50 (d, J = 7.6 Hz, 1H), 7.28- |
| | | | | 7.15 (m, 1H), 6.92-6.85 (m, 1H), 6.22 (s, 1H), 6.17- |
| | | | | 6.08 (m, 2H), 5.60 (d, J = 7.6 Hz, 1H), 4.71-4.61 (m, 1H), |
| | | | | 4.59-4.53 (m, 1H), 4.33-4.20 (m, 2H), 4.06 (s, 1H), 3.06- |
| | | | | 2.96 (m, 3H), 2.91-2.81 (m, 1H), 2.77-2.66 (m, 1H), |
| | | | | 2.54 (d, J = 3.2 Hz, 1H), 2.39 (d, J = 4.4 Hz, 4H), 2.33 (s, |
| | | | | 3H), 2.19-2.00 (m, 4H), 1.86 (s, 1H), 1.83-1.75 (m, 1H), |
| | | | | 1.72-1.60 (m, 9H), 1.55-1.47 (m, 3H), 1.37-1.24 (m, |
| | | | | 2H), 1.20-1.09 (m, 2H), 0.83-0.70 (m, 2H) |
| I-782 | OQ | PW | 835.4 | 10.80 (s, 1H), 9.56 (s, 1H), 8.39 (s, 1H), 8.07 (t, J = 8.4 Hz, |
| | | | | 1H), 7.68 (d, J = 7.2 Hz, 1H), 7.64-7.57 (m, 2H), 7.00 (d, |
| | | | | J = 14.4 Hz, 1H), 6.94-6.93 (m, 2H), 4.46 (s, 1H), 3.79 |
| | | | | (dd, J = 4.8, 11.6 Hz, 1H), 3.66-3.63 (m, 1H), 3.45-3.40 |
| | | | | (m, 1H), 3.31-3.25 (m, 2H), 2.99-2.86 (m, 5H), 2.68- |
| | | | | 2.60 (m, 1H), 2.48-2.40 (m, 4H), 2.24-2.13 (m, 1H), 2.05 |
| | | | | (d, J = 7.2 Hz, 2H), 2.02-1.93 (m, 1H), 1.82-1.73 (m, |
| | | | | 1H), 1.71-1.60 (m, 4H), 1.59-1.53 (m, 2H), 1.47-1.32 |
| | | | | (m, 2H), 1.22-1.08 (m, 3H), 1.04 (s, 3H), 0.84-0.74 (m, |
| | | | | 2H) |
| I-785 | NW | NX | 959.2 | 11.10 (s, 1H), 9.08 (s, 1H), 8.34 (s, 1H), 7.88 (d, J = 8.4 |
| | | | | Hz, 1H), 7.68-7.56 (m, 2H), 7.47 (d, J = 7.2 Hz, 1H), 6.85 |
| | | | | (d, J = 8.4 Hz, 1H), 6.78-6.65 (m, 1H), 5.40-5.25 (m, |
| | | | | 1H), 4.46 (s, 1H), 3.62-3.56 (m, 1H), 3.46 (d, J = 1.6 Hz, |
| | | | | 3H), 3.43-3.39 (m, 2H), 3.28-3.17 (m, 4H), 3.14-3.09 |
| | | | | (m, 1H), 2.92 (s, 5H), 2.68-2.62 (m, 2H), 2.59 (s, 1H), |
| | | | | 2.44 (s, 3H), 2.33 (s, 3H), 2.06 (d, J = 7.2 Hz, 2H), 2.00 |
| | | | | (dd, J = 6.4, 11.6 Hz, 1H), 1.85-1.79 (m, 2H), 1.75-1.69 |
| | | | | (m, 2H), 1.59-1.52 (m, 2H), 1.48-1.36 (m, 2H), 1.30- |
| | | | | 1.20 (m, 1H), 1.04 (s, 3H), 0.99 (d, J = 13.2 Hz, 2H), 0.92 |
| | | | | (d, J = 6.4 Hz, 3H), 0.84-0.73 (m, 2H) |
| I-786 | NW | OA | 959.6 | 11.10 (s, 1H), 9.09 (s, 1H), 8.34 (s, 1H), 7.88 (d, J = 8.4 |
| | | | | Hz, 1H), 7.66-7.56 (m, 2H), 7.48 (d, J = 7.2 Hz, 1H), 6.85 |
| | | | | (d, J = 8.8 Hz, 1H), 6.71 (t, J = 8.4 Hz, 1H), 5.33 (dd, J = |
| | | | | 5.2, 12.8 Hz, 1H), 4.47 (s, 1H), 3.62-3.55 (m, 2H), 3.46 |
| | | | | (s, 3H), 3.43 (s, 2H), 3.25 (d, J = 6.0 Hz, 3H), 3.14-3.10 |
| | | | | (m, 1H), 2.97 (d, J = 4.0 Hz, 1H), 2.91 (s, 4H), 2.70-2.62 |
| | | | | (m, 2H), 2.59 (s, 1H), 2.44 (s, 4H), 2.33 (s, 3H), 2.06 (d, J = |
| | | | | 8.8 Hz, 2H), 2.02-1.97 (m, 1H), 1.84-1.78 (m, 2H), |
| | | | | 1.75-1.68 (m, 2H), 1.60-1.52 (m, 2H), 1.48-1.35 (m, |
| | | | | 2H), 1.04 (s, 3H), 1.02-0.96 (m, 2H), 0.92 (d, J = 6.3 Hz, |
| | | | | 3H), 0.83-0.71 (m, 2H) |
| I-787 | QI | OA | 930.4 | 11.30-10.84 (m, 1H), 9.08 (s, 1H), 8.33 (s, 1H), 7.87 (d, J = |
| | | | | 8.4 Hz, 1H), 7.63 (s, 1H), 7.62-7.57 (m, 1H), 7.51-7.41 |
| | | | | (m, 1H), 7.06-6.99 (m, 1H), 6.98-6.93 (m, 1H), 5.38- |
| | | | | 5.33 (m, 1H), 4.46 (s, 1H), 3.79-3.72 (m, 1H), 3.62-3.59 |
| | | | | (m, 2H), 3.46 (s, 3H), 3.42-2.39 (m, 2H), 3.30 (s, 3H), |
| | | | | 3.27-3.23 (s, 2H), 3.13-3.10 (m, 1H), 2.98-2.96 (m, |
| | | | | 2H), 2.66-2.64 (m, 4H), 2.32 (s, 3H), 2.18-2.16 (m, 3H), |
| | | | | 2.03-1.98 (m, 1H), 1.84-1.75 (m, 3H), 1.69-1.66 (m, |
| | | | | 2H), 1.58-1.52 (m, 2H), 1.47-1.40 (m, 1H), 1.15 (s, 1H), |
| | | | | 1.04 (s, 2H), 0.98-0.94 (m, 1H), 0.92 (d, J = 6.4 Hz, 3H), |
| | | | | 0.84-0.75 (m, 2H) |
| I-788 | QK | OA | 912.4 | 11.00 (s, 1H), 9.09 (s, 1H), 8.33 (s, 1 H), 7.87 (d, J = 8.4 |
| | | | | Hz, 1H), 7.58-7.64 (m, 2H), 7.46 (d, J = 7.2 Hz, 1H), 7.16 |
| | | | | (d, J = 8.4 Hz, 1H), 6.99-7.06 (m, 2H), 5.36 (m, 1H), 4.46 |
| | | | | (s, 1H), 4.14-4.21 (m, 1H), 3.53-3.59 (m, 3H), 3.49 (s, |
| | | | | 3H), 3.41 (d, J = 12.8 Hz, 1H), 3.30 (s, 2H), 3.27 (s, 1H), |
| | | | | 3.23 (s, 1H), 3.16-3.21 (m, 1H), 3.12 (d, J = 6.4 Hz, 3H), |
| | | | | 2.93-3.00 (m, 1H), 2.87 (m, 1H), 2.63-2.71 (m, 2H), 2.32 |
| | | | | (s, 3H), 2.20 (d, J = 6.8 Hz, 2H), 1.97-2.01 (m, 1H), 1.78- |
| | | | | 1.83 (m, 2H), 1.67-1.73 (m, 2H), 1.53-1.59 (m, 2H), |
| | | | | 1.44 (m, 1H), 1.23 (s, 2H), 1.04 (s, 3H), 0.97 (d, J = 12.0 |
| | | | | Hz, 1H), 0.91 (d, J = 6.4 Hz, 3H), 0.78-0.85 (m, 2H) |
| I-789 | QK | QL | 926.4 | 11.09 (s, 1H), 8.94 (s, 1H), 8.30 (s, 1H), 7.97 (d, J = 8.4 |
| | | | | Hz, 1H), 7.55-7.67 (m, 2H), 7.44 (d, J = 6.8 Hz, 1H), 7.15 |
| | | | | (d, J = 7.6 Hz, 1H), 6.95-7.10 (m, 2H), 5.29-5.45 (m, |
| | | | | 1H), 4.43 (s, 1H), 4.16 (t, J = 7.2 Hz, 1H), 3.63-3.68 (m, |
| | | | | 1H), 3.52-3.57 (m, 3H), 3.49 (s, 3H), 3.43 (d, J = 12.0 Hz, |
| | | | | 2H), 3.17-3.25 (m, 2H), 3.11 (d, J = 7.2 Hz, 2H), 2.86- |
| | | | | 2.95 (m, 2H), 2.57-2.73 (m, 5H), 2.33 (s, 3H), 2.16 (d, J = |
| | | | | 6.4 Hz, 2H), 1.95-2.01 (m, 1H), 1.75-1.88 (m, 4H), |
| | | | | 1.68 (d, J = 11.2 Hz, 2H), 1.11 (d, J = 4.8 Hz, 6H), 0.94- |
| | | | | 1.01 (m, 2H), 0.92 (d, J = 6.4 Hz, 3H), 0.70-0.85 (m, 2H) |
| I-790 | NZ | ND | 940.6 | 11.08 (s, 1H), 9.62 (s, 1H), 8.60 (s, 1H), 7.81 (d, J = 8.4 |
| | | | | Hz, 1H), 7.67 (d, J = 1.6 Hz, 1H), 7.61 (dd, J = 2.0, 8.4 Hz, |
| | | | | 1H), 7.50 (d, J = 7.2 Hz, 1H), 7.01-6.94 (m, 1H), 6.93- |
| | | | | 6.84 (m, 2H), 5.34 (dd, J = 5.6, 12.4 Hz, 1H), 4.00 (s, 1H), |
| | | | | 3.60 (s, 3H), 3.22-3.18 (m, 1H), 3.17-3.09 (m, 2H), 3.05- |
| | | | | 2.96 (m, 2H), 2.95-2.81 (m, 5H), 2.75-2.56 (m, 5H), |
| | | | | 2.33 (s, 3H), 2.16-2.03 (m, 5H), 2.02-1.96 (m, 1H), 1.84 |
| | | | | (d, J = 8.8 Hz, 2H), 1.73 (d, J = 11.2 Hz, 2H), 1.63 (d, J = |
| | | | | 12.8 Hz, 2H), 1.46 (d, J = 11.6 Hz, 2H), 1.41-1.29 (m, |
| | | | | 3H), 1.13 (s, 3H), 1.06-0.98 (m, 2H), 0.95 (d, J = 6.4 Hz, |
| | | | | 3H), 0.86-0.74 (m, 2H) |
| I-791 | QP | ND | 940.4 | 11.18-10.94 (m, 1H), 9.64 (s, 1H), 8.78-8.46 (m, 1H), |
| | | | | 7.81 (d, J = 8.0 Hz, 1H), 7.72-7.57 (m, 2H), 7.56-7.46 |
| | | | | (m, 1H), 7.01-6.77 (m, 2H), 6.68-6.53 (m, 1H), 5.37- |
| | | | | 5.21 (m, 1H), 4.13-3.87 (m, 1H), 3.30 (s, 3H), 3.09-2.97 |
| | | | | (m, 6H), 2.96-2.82 (m, 2H), 2.75-2.65 (m, 2H), 2.61 (d, |
| | | | | J = 16.5 Hz, 1H), 2.47-2.41 (m, 4H), 2.34 (s, 3H), 2.13- |
| | | | | 2.04 (m, 4H), 1.99 (d, J = 8.4 Hz, 1H), 1.88-1.79 (m, 2H), |
| | | | | 1.79-1.70 (m, 2H), 1.68-1.60 (m, 2H), 1.54-1.30 (m, |
| | | | | 6H), 1.14 (s, 3H), 1.08-0.93 (m, 5H), 0.82-0.74 (m, 2H) |
| I-792^{b} | NW | ON | 957.4 | 11.10 (s, 1H), 8.83 (s, 1H), 8.02-7.89 (m, 2H), 7.66-7.53 |
| | | | | (m, 2H), 7.45 (d, J = 7.2 Hz, 1H), 6.85 (d, J = 8.4 Hz, 1H), |
| | | | | 6.70 (t, J = 8.4 Hz, 1H), 5.33 (dd, J = 5.2, 12.8 Hz, 1H), |
| | | | | 4.73-4.62 (m, 1H), 4.56 (d, J = 2.4 Hz, 1H), 4.07 (s, 1H), |
| | | | | 3.46 (d, J = 1.6 Hz, 3H), 3.26-3.09 (m, 4H), 2.97-2.83 |
| | | | | (m, 6H), 2.74-2.57 (m, 3H), 2.45-2.39 (m, 4H), 2.35 (s, |
| | | | | 3H), 2.21-2.12 (m, 1H), 2.06-1.95 (m, 3H), 1.85-1.77 |
| | | | | (m, 2H), 1.76-1.70 (m, 2H), 1.69-1.57 (m, 6H), 1.55- |
| | | | | 1.47 (m, 3H), 1.41-1.25 (m, 2H), 1.03-0.97 (m, 1H), 0.94 |
| | | | | (d, J = 6.4 Hz, 3H), 0.82-0.69 (m, 2H) |
| I-793 | QR | OA | 913.2 | 11.09 (s, 1H), 9.10 (s, 1H), 8.33 (s, 1H), 7.87 (d, J = 8.4 |
| | | | | Hz, 1H), 7.64 (s, 1H), 7.62-7.57 (m, 1H), 7.52 (d, J = 7.6 |
| | | | | Hz, 1H), 7.01-6.95 (m, 1H), 6.93-6.85 (m, 2H), 5.37- |
| | | | | 5.32 (m, 1H), 4.46 (s, 1H), 3.92-3.80 (m, 1H), 3.60 (s, |
| | | | | 3H), 3.28-3.24 (m, 3H), 3.12-3.07 (m, 1H), 3.02-2.99 |
| | | | | (m, 1H), 2.92-2.69 (m, 6H), 2.69-2.65 (m, 2H), 2.64- |
| | | | | 2.50 (m, 1H), 2.33 (s, 8H), 2.24-2.13 (m, 2H), 2.03-1.95 |
| | | | | (m, 1H), 1.89-1.82 (m, 4H), 1.80-1.71 (m, 1H), 1.59- |
| | | | | 1.52 (m, 2H), 1.48-1.40 (m, 1H), 1.03 (s, 3H), 0.94 (d, J = |
| | | | | 6.8 Hz, 3H) |
| I-794 | QT | ND | 912.6 | 11.09 (s, 1H), 9.63 (s, 1H), 8.60 (s, 1H), 7.81 (d, J = 8.4 |
| | | | | Hz, 1H), 7.67 (s, 1H), 7.64-7.51 (m, 2H), 7.02-6.80 (m, |
| | | | | 3H), 5.34 (dd, J = 5.2, 12.8 Hz, 1H), 4.00 (s, 1H), 3.69- |
| | | | | 3.62 (m, 1H), 3.59 (s, 3H), 3.16-3.08 (m, 1H), 3.01 (dd, J = |
| | | | | 6.4, 9.6 Hz, 1H), 2.96-2.75 (m, 6H), 2.75-2.57 (m, 5H), |
| | | | | 2.37-2.30 (m, 5H), 2.22 (dd, J = 5.6, 11.6 Hz, 3H), 2.14- |
| | | | | 2.05 (m, 2H), 2.04-1.83 (m, 3H), 1.62 (d, J = 12.4 Hz, |
| | | | | 2H), 1.48-1.28 (m, 6H), 1.13 (s, 3H), 0.95 (d, J = 6.4 Hz, |
| | | | | 3H) |
| I-795 | QW | QX | 908.3 | 11.08 (s, 1H), 9.15 (s, 1H), 8.37 (s, 1H), 7.90-7.72 (m, |
| | | | | 2H), 7.63-7.50 (m, 2H), 7.02-6.80 (m, 3H), 5.40-5.27 |
| | | | | (m, 1H), 3.60 (s, 4H), 3.45 (s, 4H), 3.09 (d, J = 9.6 Hz, 2H), |
| | | | | 2.93-2.83 (m, 1H), 2.77-2.55 (m, 8H), 2.31 (s, 6H), 2.23- |
| | | | | 2.10 (m, 4H), 1.99 (d, J = 5.6 Hz, 3H), 1.86-1.73 (m, |
| | | | | 4H), 1.63-1.49 (m, 7H), 1.34-1.21 (m, 2H) |
| I-796 | QW | RC | 958.4 | 11.29-10.76 (m, 1H), 9.22 (s, 1H), 8.42 (s, 1H), 7.86 (d, J = |
| | | | | 6.8 Hz, 1H), 7.81 (d, J = 8.4 Hz, 1H), 7.61 (d, J = 1.6 Hz, |
| | | | | 1H), 7.54 (dd, J = 2.0, 8.4 Hz, 1H), 7.00-6.93 (m, 1H), |
| | | | | 6.90-6.83 (m, 2H), 5.94 (d, J = 4.4 Hz, 1H), 5.35 (dd, J = |
| | | | | 5.2, 12.4 Hz, 1H), 4.10 (d, J = 12.0 Hz, 1H), 3.92 (d, J = |
| | | | | 14.4 Hz, 1H), 3.66-3.60 (m, 3H), 3.60-3.55 (m, 1H), 3.15- |
| | | | | 3.05 (m, 2H), 3.01-2.92 (m, 2H), 2.89 (d, J = 5.6 Hz, |
| | | | | 1H), 2.74-2.57 (m, 6H), 2.33 (s, 8H), 2.16 (d, J = 6.4 Hz, |
| | | | | 4H), 2.05-1.93 (m, 4H), 1.87-1.72 (m, 6H), 1.66-1.55 |
| | | | | (m, 1H), 1.53-1.37 (m, 2H), 1.35-1.21 (m, 2H) |
| I-797 | QW | RB | 958.4 | 11.14-11.03 (m, 1H), 9.25-9.14 (m, 1H), 8.41 (s, 1H), |
| | | | | 7.86-7.78 (m, 2H), 7.64-7.49 (m, 2H), 6.99-6.93 (m, 1H), |
| | | | | 6.90-6.82 (m, 2H), 6.12-5.75 (m, 1H), 5.39-5.29 (m, |
| | | | | 1H), 4.14-4.04 (m, 1H), 3.95-3.85 (m, 1H), 3.64-3.59 |
| | | | | (m, 3H), 3.58-3.53 (m, 1H), 3.28-3.21 (m, 1H), 3.14- |
| | | | | 3.03 (m, 2H), 2.99-2.91 (m, 2H), 2.90-2.83 (m, 1H), 2.72- |
| | | | | 2.55 (m, 6H), 2.32 (s, 5H), 2.23-2.06 (m, 6H), 2.04- |
| | | | | 1.93 (m, 4H), 1.85-1.71 (m, 6H), 1.65-1.56 (m, 1H), 1.52- |
| | | | | 1.38 (m, 2H), 1.33-1.21 (m, 2H) |
| I-798 | RK | OA | 984.5 | 11.11 (s, 1H), 9.10 (s, 1H), 8.34 (s, 1H), 7.84 (d, J = 8.4 |
| | | | | Hz, 1H), 7.74 (d, J = 9.2 Hz, 1H), 7.60 (s, 1H), 7.56 (dd, J = |
| | | | | 2.0, 8.4 Hz, 1H), 7.06-6.96 (m, 2H), 5.36 (dd, J = 5.6, |
| | | | | 12.8 Hz, 1H), 4.46 (s, 1H), 3.60 (s, 3H), 3.49-3.40 (m, |
| | | | | 3H), 3.38 (s, 2H), 3.29-3.23 (m, 2H), 3.15 (s, 1H), 3.05 |
| | | | | (s, 1H), 2.92 (d, J = 9.6 Hz, 2H), 2.88-2.81 (m, 1H), 2.74- |
| | | | | 2.57 (m, 5H), 2.39 (dd, J = 8.8, 14.8 Hz, 3H), 2.32 (s, 3H), |
| | | | | 2.28-2.22 (m, 1H), 2.11-2.05 (m, 1H), 2.04-1.95 (m, |
| | | | | 2H), 1.80-1.75 (m, 1H), 1.78-1.70 (m, 2H), 1.59-1.51 |
| | | | | (m, 4H), 1.50-1.36 (m, 4H), 1.28-1.20 (m, 2H), 1.04 (s, |
| | | | | 3H |
| I-799 | RL | OA | 813.2 | 10.76 (s, 1H), 9.08 (s, 1H), 8.34 (s, 1H), 7.82 (d, J = 8.4 |
| | | | | Hz, 1H), 7.67-7.48 (m, 3H), 7.03 (d, J = 8.4 Hz, 2H), 6.86 |
| | | | | (d, J = 8.8 Hz, 2H), 4.44 (s, 1H), 3.76-3.68 (m, 1H), 3.65- |
| | | | | 3.54 (m, 1H), 3.09-3.03 (m, 4H), 2.90-2.83 (m, 1H), |
| | | | | 2.42-2.39 (m, 5H), 2.32 (s, 6H), 2.15-2.07 (m, 2H), 2.04 |
| | | | | (d, J = 7.6 Hz, 2H), 1.73-1.61 (m, 5H), 1.58-1.51 (m, |
| | | | | 2H), 1.46-1.31 (m, 3H), 1.19-1.10 (m, 2H), 1.03 (s, 3H), |
| | | | | 0.83-0.72 (m, 2H) |
| I-800 | RN | OA | 865.2 | 10.86 (s, 1H), 9.08 (s, 1H), 8.34 (s, 1H), 7.82 (d, J = 8.4 |
| | | | | Hz, 1H), 7.69-7.56 (m, 2H), 7.51 (d, J = 7.6 Hz, 1H), 7.17 |
| | | | | (d, J = 13.6 Hz, 1H), 6.99 (d, J = 8.4 Hz, 1H), 4.45 (s, 1H), |
| | | | | 4.10 (dd, J = 4.8, 12.8 Hz, 1H), 3.64-3.57 (m, 1H), 3.25- |
| | | | | 3.21 (m, 2H), 2.99 (s, 4H), 2.90-2.83 (m, 1H), 2.78-2.71 |
| | | | | (m, 1H), 2.53 (s, 2H), 2.42 (s, 4H), 2.32 (s, 3H), 2.27 (dd, |
| | | | | J = 4.4, 13.2 Hz, 1H), 2.08-2.01 (m, 2H), 1.96-1.88 (m, |
| | | | | 1H), 1.81-1.73 (m, 1H), 1.71-1.61 (m, 4H), 1.59-1.50 |
| | | | | (m, 2H), 1.45-1.31 (m, 2H), 1.18-1.08 (m, 2H), 1.03 (s, |
| | | | | 3H), 0.83-0.71 (m, 2H) |
| I-802 | RS | OA | 839.4 | 10.76 (s, 1H), 9.09 (s, 1H), 8.33 (s, 1H), 7.77 (d, J = 8.8 |
| | | | | Hz, 1H), 7.63 (s, 1H), 7.59 (dd, J = 2.0, 8.4 Hz, 1H), 7.34 |
| | | | | (s, 1H), 7.02 (d, J = 8.8 Hz, 2H), 6.84 (d, J = 8.8 Hz, 2H), |
| | | | | 4.43 (s, 1H), 3.71 (dd, J = 4.8, 11.2 Hz, 1H), 3.65-3.56 |
| | | | | (m, 1H), 3.42 (d, J = 13.2 Hz, 1H), 3.25-3.17 (m, 2H), |
| | | | | 3.03 (s, 4H), 2.68-2.56 (m, 1H), 2.42 (t, J = 4.4 Hz, 3H), |
| | | | | 2.31 (s, 3H), 2.17-2.05 (m, 1H), 2.04-1.92 (m, 3H), 1.81- |
| | | | | 1.69 (m, 1H), 1.63-1.49 (m, 9H), 1.44-1.29 (m, 8H), |
| | | | | 1.02 (s, 3H) |
| I-803 | RW | OA | 845.1 | 10.86-10.78 (m, 1H), 9.14-9.05 (m, 1H), 8.34 (s, 1H), |
| | | | | 7.79 (d, J = 8.4 Hz, 1H), 7.71-7.57 (m, 2H), 7.35 (s, 1H), |
| | | | | 7.08-6.90 (m, 3H), 4.54-4.38 (m, 1H), 3.80 (dd, J = 4.8, |
| | | | | 11.6 Hz, 1H), 3.65-3.56 (m, 1H), 3.42 (d, J = 13.2 Hz, |
| | | | | 1H), 3.27-3.20 (m, 2H), 2.97 (s, 4H), 2.69-2.61 (m, 1H), |
| | | | | 2.55-2.53 (m, 1H), 2.48-2.41 (m, 4H), 2.32 (s, 3H), 2.26- |
| | | | | 2.16 (m, 1H), 2.15 (s, 1H), 2.11 (d, J = 7.2 Hz, 1H), 2.03- |
| | | | | 1.93 (m, 1H), 1.81-1.70 (m, 1H), 1.64-1.52 (m, 6H), |
| | | | | 1.50-1.34 (m, 4H), 1.10 (s, 3H), 1.03 (s, 3H), 1.01-0.88 |
| | | | | (m, 2H) |
| I-804 | RY | OA | 849 | 10.80 (s, 1H), 9.08 (s, 1H), 8.34 (s, 1H), 7.94-7.79 (m, |
| | | | | 2H), 7.70-7.53 (m, 2H), 7.06-6.85 (m, 3H), 4.45 (s, 1H), |
| | | | | 4.37-4.13 (m, 1H), 3.84-3.72 (m, 1H), 3.66-3.53 (m, |
| | | | | 1H), 3.44-3.37 (m, 1H), 3.30-3.20 (m, 4H), 3.16-2.98 |
| | | | | (m, 2H), 2.67-2.56 (m, 4H), 2.48-2.42 (m, 1H), 2.32 (s, |
| | | | | 3H), 2.23-2.13 (m, 3H), 2.04-1.95 (m, 2H), 1.93-1.85 |
| | | | | (m, 1H), 1.79-1.67 (m, 3H), 1.63-1.51 (m, 4H), 1.47- |
| | | | | 1.32 (m, 2H), 1.26-1.15 (m, 2H), 1.04 (s, 3H) |
| I-805 | RZ | OA | 859.3 | 10.80 (s, 1H), 9.10 (s, 1H), 8.34 (s, 1H), 7.81 (d, J = 8.4 |
| | | | | Hz, 1H), 7.67-7.58 (m, 3H), 6.99 (d, J = 14.4 Hz, 1H), |
| | | | | 6.96-6.90 (m, 2H), 4.45 (s, 1H), 3.80-3.76 (m, 1H), 3.65- |
| | | | | 3.53 (m, 3H), 3.43 (s, 1H), 3.24-3.21 (m, 2H), 2.92 (s, |
| | | | | 4H), 2.68-2.59 (m, 1H), 2.54 (s, 4H), 2.46-2.44 (m, 1H), |
| | | | | 2.32 (s, 3H), 2.23-2.12 (m, 3H), 2.01-1.96 (m, 1H), 1.83- |
| | | | | 1.71 (m, 5H), 1.68-1.60 (m, 2H), 1.59-1.46 (m, 4H), |
| | | | | 1.45-1.37 (m, 1H), 1.03 (s, 3H) |
| I-806 | SA | OA | 845.5 | 10.83 (s, 1H), 9.09 (s, 1H), 8.34 (s, 1H), 7.83 (d, J = 8.4 |
| | | | | Hz, 1H), 7.64 (s, 1H), 7.61-7.56 (m, 1H), 7.52 (d, J = 7.2 |
| | | | | Hz, 1H), 7.04 (d, J = 8.8 Hz, 1H), 6.77 (d, J = 12.4 Hz, 1H), |
| | | | | 4.45 (s, 1H), 3.90 (dd, J = 4.8, 12.4 Hz, 1H), 3.65-3.55 |
| | | | | (m, 1H), 3.43-3.38 (m, 1H), 3.29-3.19 (m, 3H), 2.91- |
| | | | | 2.75 (m, 5H), 2.73-2.65 (m, 1H), 2.47-2.37 (m, 4H), 2.32 |
| | | | | (s, 3H), 2.20-2.11 (m, 4H), 2.10-2.02 (m, 2H), 2.00- |
| | | | | 1.89 (m, 1H), 1.81-1.72 (m, 1H), 1.72-1.60 (m, 4H), 1.59- |
| | | | | 1.48 (m, 2H), 1.46-1.30 (m, 2H), 1.19-1.07 (m, 2H), |
| | | | | 1.03 (s, 3H), 0.86-0.70 (m, 2H) |
| I-807 | SC | OA | 847.6 | 10.82 (s, 1H), 9.09 (s, 1H), 8.34 (s, 1H), 7.83 (d, J = 8.4 |
| | | | | Hz, 1H), 7.64 (s, 1H), 7.61-7.57 (m, 1H), 7.51 (d, J = 7.2 |
| | | | | Hz, 1H), 7.27 (d, J = 2.0 Hz, 1H), 7.15-7.11 (m, 1H), 7.10- |
| | | | | 7.06 (m, 1H), 4.45 (s, 1H), 3.81 (dd, J = 4.8, 12.0 Hz, 1H), |
| | | | | 3.65-3.56 (m, 1H), 3.44-3.37 (m, 2H), 3.27-3.22 (m, |
| | | | | 2H), 2.97-2.82 (m, 5H), 2.70-2.58 (m, 1H), 2.46-2.44 |
| | | | | (m, 4H), 2.32 (s, 3H), 2.25-2.14 (m, 1H), 2.07-2.05 (m, |
| | | | | 2H), 2.02-1.94 (m, 1H), 1.81-1.72 (m, 1H), 1.70-1.63 |
| | | | | (m, 4H), 1.59-1.52 (m, 2H), 1.46-1.31 (m, 2H), 1.20- |
| | | | | 1.07 (m, 2H), 1.03 (s, 3H), 0.85-0.71 (m, 2H) |
| I-808 | SF | OA | 845.4 | 10.77 (s, 1H), 9.08 (s, 1H), 8.34 (s, 1H), 7.81 (d, J = 8.4 |
| | | | | Hz, 1H), 7.69-7.59 (m, 2H), 7.21 (s, 1H), 7.03 (d, J = 8.8 |
| | | | | Hz, 2H), 6.90 (d, J = 8.8 Hz, 2H), 4.45 (s, 1H), 3.72 (dd, J = |
| | | | | 4.8, 10.8 Hz, 1H), 3.59 (dd, J = 5.2, 8.0 Hz, 1H), 3.41 (d, |
| | | | | J = 13.2 Hz, 4H), 3.30-3.17 (m, 3H), 2.93 (t, J = 10.8 Hz, |
| | | | | 2H), 2.70-2.54 (m, 2H), 2.48-2.35 (m, 6H), 2.32 (s, 3H), |
| | | | | 2.19-2.06 (m, 1H), 2.05-1.95 (m, 1H), 1.87-1.76 (m, |
| | | | | 5H), 1.60-1.50 (m, 2H), 1.45-1.34 (m, 3H), 1.02 (d, J = |
| | | | | 5.2 Hz, 6H) |
| I-810 | SH | OA | 845.4 | 10.80 (s, 1H), 9.09 (s, 1H), 8.34 (s, 1H), 7.82 (d, J = 8.4 |
| | | | | Hz, 1H), 7.66-7.56 (m, 2H), 7.50 (d, J = 7.2 Hz, 1H), 7.03- |
| | | | | 6.89 (m, 3H), 4.44 (s, 1H), 3.80-3.76 (m, 1H), 3.63- |
| | | | | 3.55 (m, 1H), 3.39 (s, 2H), 3.26-3.22 (m, 2H), 3.08 (d, J = |
| | | | | 8.0 Hz, 2H), 2.89-2.75 (m, 3H), 2.69-2.58 (m, 2H), |
| | | | | 2.47-2.40 (m, 2H), 2.37 (d, J = 12.0 Hz, 1H), 2.32 (s, 3H), |
| | | | | 2.26-2.15 (m, 2H), 2.00-1.96 (m, 1H), 1.89-1.85 (m, |
| | | | | 1H), 1.82-1.73 (m, 2H), 1.66-1.60 (m, 2H), 1.59-1.53 |
| | | | | (m, 2H), 1.47-1.39 (m, 1H), 1.34-1.26 (m, 1H), 1.17- |
| | | | | 1.06 (m, 2H), 1.03 (s, 3H), 0.97 (d, J = 6.0 Hz, 3H), 0.88- |
| | | | | 0.78 (m, 1H), 0.77-0.63 (m, 1H) |
| I-811 | SJ | OA | 827.5 | 10.78 (s, 1H), 9.10 (s, 1H), 8.35 (s, 1H), 7.83 (d, J = 8.4 |
| | | | | Hz, 1H), 7.65 (d, J = 1.6 Hz, 1H), 7.60 (dd, J = 2.0, 8.4 Hz, |
| | | | | 1H), 7.52 (d, J = 7.2 Hz, 1H), 6.97 (d, J = 11.2 Hz, 3H), |
| | | | | 4.46 (s, 1H), 3.73 (dd, J = 5.2, 11.2 Hz, 1H), 3.65-3.57 |
| | | | | (m, 1H), 3.40 (s, 1H), 3.26 (d, J = 13.2 Hz, 2H), 2.93-2.85 |
| | | | | (m, 1H), 2.79 (s, 4H), 2.68 (s, 2H), 2.44 (d, J = 4.4 Hz, 4H), |
| | | | | 2.33 (s, 3H), 2.20 (s, 3H), 2.16-2.10 (m, 1H), 2.07 (d, J = |
| | | | | 7.2 Hz, 2H), 2.02 (d, J = 4.4 Hz, 1H), 1.81-1.74 (m, 1H), |
| | | | | 1.68 (s, 4H), 1.61-1.52 (m, 2H), 1.48-1.33 (m, 2H), 1.14 |
| | | | | (d, J = 12.4 Hz, 2H), 1.04 (s, 3H), 0.78 (d, J = 12.4 Hz, 2H) |
| I-812 | SL | OA | 845.2 | 10.82 (s, 1H), 9.09 (s, 1H), 8.34 (s, 1H), 7.82 (d, J = 8.4 |
| | | | | Hz, 1H), 7.64 (d, J = 2.0 Hz, 1H), 7.59 (dd, J = 2.0, 8.4 Hz, |
| | | | | 1H), 7.52 (d, J = 7.2 Hz, 1H), 6.87-6.77 (m, 2H), 4.45 (s, |
| | | | | 1H), 3.99 (dd, J = 4.8, 12.0 Hz, 1H), 3.64-3.57 (m, 1H), |
| | | | | 3.42 (s, 1H), 3.27-3.22 (m, 3H), 2.94 (s, 4H), 2.89-2.83 |
| | | | | (m, 1H), 2.75-2.67 (m, 1H), 2.43 (s, 4H), 2.32 (s, 3H), |
| | | | | 2.16 (dd, J = 4.0, 12.8 Hz, 1H), 2.10 (d, J = 2.4 Hz, 3H), |
| | | | | 2.08-2.04 (m, 2H), 1.98-1.91 (m, 1H), 1.80-1.72 (m, |
| | | | | 1H), 1.66 (t, J = 13.6 Hz, 4H), 1.59-1.50 (m, 2H), 1.45- |
| | | | | 1.31 (m, 2H), 1.18-1.06 (m, 2H), 1.03 (s, 3H), 0.83-0.71 |
| | | | | (m, 2H) |
| I-813 | SM | OA | 901.3 | 11.18-11.06 (m, 1H), 9.10 (s, 1H), 8.35 (s, 1H), 7.86 (d, J = |
| | | | | 2.0 Hz, 1H), 7.64 (s, 1H), 7.60 (dd, J = 2.0, 9.2 Hz, 1H), |
| | | | | 7.55-7.50 (m, 1H), 6.85 (d, J = 8.4 Hz, 1H), 6.75-6.68 |
| | | | | (m, 1H), 5.37 (s, 1H), 4.46 (s, 1H), 3.65-3.57 (m, 1H), |
| | | | | 3.46 (d, J = 0.8 Hz, 3H), 3.45-3.36 (m, 2H), 3.29-3.22 |
| | | | | (m, 2H), 2.94-2.83 (m, 6H), 2.72-2.61 (m, 2H), 2.46- |
| | | | | 2.42 (m, 3H), 2.33 (s, 3H), 2.11-2.04 (m, 2H), 2.04-1.96 |
| | | | | (m, 1H), 1.82-1.74 (m, 1H), 1.73-1.62 (m, 4H), 1.60- |
| | | | | 1.52 (m, 2H), 1.49-1.32 (m, 2H), 1.20-1.08 (m, 2H), 1.07- |
| | | | | 1.02 (m, 3H), 0.85-0.72 (m, 2H) |
| I-814 | SN | OA | 845.1 | 10.81 (s, 1H), 9.08 (s, 1H), 8.34 (s, 1H), 7.81 (d, J = 8.4 |
| | | | | Hz, 1H), 7.69-7.59 (m, 2H), 7.01 (t, J = 6.8 Hz, 2H), 6.94 |
| | | | | (d, J = 4.0 Hz, 2H), 4.45 (s, 1H), 3.83-3.75 (m, J = 4.8, |
| | | | | 11.6 Hz, 1H), 3.66-3.54 (m, 1H), 3.41 (d, J = 13.2 Hz, |
| | | | | 1H), 3.23 (d, J = 13.2 Hz, 2H), 2.96 (s, 4H), 2.68-2.59 (m, |
| | | | | 1H), 2.47-2.41 (m, 4H), 2.32 (s, 3H), 2.25-2.15 (m, 1H), |
| | | | | 2.09 (d, J = 6.0 Hz, 2H), 2.03-1.91 (m, 3H), 1.82-1.71 |
| | | | | (m, 1H), 1.59-1.52 (m, 2H), 1.43 (s, 4H), 1.29-1.07 (m, |
| | | | | 5H), 1.03 (s, 3H), 0.94 (s, 3H) |
| I-815 | SP | OA | 849.3 | 10.80 (s, 1H), 9.09 (s, 1H), 8.34 (s, 1H), 7.83 (d, J = 8.8 |
| | | | | Hz, 1H), 7.65 (s, 1H), 7.63-7.55 (m, 2H), 7.04-6.90 (m, |
| | | | | 3H), 4.45 (s, 1H), 3.79 (dd, J = 5.2, 11.6 Hz, 1H), 3.65- |
| | | | | 3.56 (m, 1H), 3.42-3.37 (m, 2H), 3.27-3.22 (m, 3H), 2.99- |
| | | | | 2.92 (m, 4H), 2.70-2.59 (m, 2H), 2.58-2.54 (m, 3H), |
| | | | | 2.45 (s, 1H), 2.39 (s, 1H), 2.32 (s, 3H), 2.23-2.13 (m, 1H), |
| | | | | 2.03-1.94 (m, 1H), 1.90-1.80 (m, 2H), 1.78-1.68 (m, |
| | | | | 1H), 1.59-1.46 (m, 4H), 1.46-1.35 (m, 4H), 1.34-1.22 |
| | | | | (m, 1H), 1.03 (s, 3H) |
| I-816 | SS | OA | 876.3 | 10.85 (s, 1H), 9.10 (s, 1H), 8.34 (s, 1H), 7.83 (d, J = 8.4 |
| | | | | Hz, 1H), 7.64 (s, 1H), 7.59 (d, J = 8.0 Hz, 1H), 7.52 (d, J = |
| | | | | 7.2 Hz, 1H), 6.60-6.50 (m, 2H), 5.09 (d, J = 6.4 Hz, 1H), |
| | | | | 4.46 (s, 1H), 4.29-4.18 (m, 1H), 3.77 (s, 3H), 3.65-3.57 |
| | | | | (m, 1H), 3.43-3.37 (m, 2H), 3.27-3.22 (m, 2H), 2.92- |
| | | | | 2.81 (m, 5H), 2.81-2.74 (m, 1H), 2.55 (d, J = 3.6 Hz, 2H), |
| | | | | 2.45-2.39 (m, 2H), 2.32 (s, 3H), 2.16-2.01 (m, 3H), 1.95- |
| | | | | 1.82 (m, 1H), 1.80-1.72 (m, 1H), 1.71-1.61 (m, 4H), |
| | | | | 2H), 1.03 (s, 3H), 0.84-0.72 (m, 2H) |
| | | | | 1.59-1.52 (m, 2H), 1.47-1.36 (m, 2H), 1.18-1.09 (m, |
| | | | | 2H), 1.03 (s, 3H), 0.84-0.72 (m, 2H) |
| I-820 | SV | OA | 843.4 | 10.80 (s, 1H), 9.11 (s, 1H), 8.33 (s, 1H), 7.93-7.73 (m, |
| | | | | 2H), 7.69-7.54 (m, 2H), 7.05 (t, J = 8.8 Hz, 1H), 6.74- |
| | | | | 6.53 (m, 2H), 4.61-4.27 (m, 1H), 3.87 (dd, J = 4.8, 12.4 |
| | | | | Hz, 1H), 3.65-3.56 (m, 1H), 3.44-3.39 (m, 2H), 3.25- |
| | | | | 3.21 (m, 2H), 3.06 (s, 4H), 2.78-2.63 (m, 1H), 2.44 (s, |
| | | | | 4H), 2.36-2.24 (m, 5H), 2.20-2.06 (m, 1H), 2.00-1.88 |
| | | | | (m, 1H), 1.76 (dt, J = 4.0, 8.4 Hz, 1H), 1.69-1.35 (m, 9H), |
| | | | | 1.30-1.14 (m, 4H), 1.03 (s, 3H) |
| I-821^{b} | SW | ON | 844.4 | 10.86-10.68 (m, 1H), 8.83 (s, 1H), 7.97-7.86 (m, 2H), |
| | | | | 7.62 (s, 1H), 7.59-7.54 (m, 1H), 7.50 (d, J = 7.2 Hz, 1H), |
| | | | | 6.79 (t, J = 9.2 Hz, 1H), 6.52-6.46 (m, 1H), 6.42-6.36 |
| | | | | (m, 1H), 5.78 (d, J = 7.6 Hz, 1H), 4.71-4.61 (m, 1H), 4.55 |
| | | | | (d, J = 2.8 Hz, 1H), 4.29-4.18 (m, 1H), 4.07 (s, 1H), 2.91- |
| | | | | 2.76 (m, 5H), 2.73-2.66 (m, 1H), 2.58 (t, J = 4.0 Hz, 1H), |
| | | | | 2.55-2.53 (m, 1H), 2.47-2.37 (m, 5H), 2.34 (s, 3H), 2.24- |
| | | | | 2.13 (m, 1H), 2.04 (d, J = 7.2 Hz, 2H), 1.88-1.81 (m, |
| | | | | 1H), 1.74-1.58 (m, 10H), 1.55-1.47 (m, 3H), 1.40-1.28 |
| | | | | (m, 2H), 1.13 (q, J = 11.2 Hz, 2H), 0.84-0.69 (m, 2H) |
| I-822 | SW | OA | 846.2 | 10.80-10.73 (m, 1H), 9.09 (s, 1H), 8.34 (s, 1H), 7.82 (d, J = |
| | | | | 8.4 Hz, 1H), 7.64 (d, J = 1.6 Hz, 1H), 7.61-7.56 (m, 1H), |
| | | | | 7.51 (d, J = 7.2 Hz, 1H), 6.79 (t, J = 9.2 Hz, 1H), 6.53- |
| | | | | 6.46 (m, 1H), 6.42-6.37 (m, 1H), 5.79 (d, J = 7.6 Hz, 1H), |
| | | | | 4.45 (s, 1H), 4.29-4.20 (m, 1H), 3.65-3.56 (m, 1H), 3.40 |
| | | | | (d, J = 13.2 Hz, 1H), 3.26 (s, 1H), 3.23 (s, 1H), 2.81 (s, |
| | | | | 4H), 2.77-2.65 (m, 2H), 2.60-2.56 (m, 1H), 2.39 (s, 4H), |
| | | | | 2.32 (s, 3H), 2.10-2.01 (m, 3H), 1.88-1.81 (m, 1H), 1.80- |
| | | | | 1.74 (m, 1H), 1.70-1.61 (m, 4H), 1.58-1.53 (m, 2H), |
| | | | | 1.45-1.39 (m, 1H), 1.37-1.30 (m, 1H), 1.17-1.08 (m, |
| | | | | 2H), 1.03 (s, 3H), 0.81-0.71 (m, 2H) |
| I-1029 | TI | PW | 879.4 | 10.79 (s, 1H), 9.56 (s, 1H), 8.39 (s, 1H), 8.09-8.05 (m, |
| | | | | 1H), 7.74-7.52 (m, 3H), 7.09-7.05 (m, 1H), 6.74-6.51 |
| | | | | (m, 2H), 4.46 (s, 1H), 3.89-3.85 (m, 1H), 3.69-3.63 (m, |
| | | | | 1H), 3.47-3.39 (m, 2H), 3.32-3.25 (m, 4H), 3.23 (s, 3H), |
| | | | | 2.99-2.87 (m, 2H), 2.85-2.65 (m, 3H), 2.52 (s, 2H), 2.48 |
| | | | | (s, 1H), 2.45-2.40 (m, 1H), 2.26-2.14 (m, 1H), 2.19- |
| | | | | 1.89 (m, 3H), 1.86-1.70 (m, 2H), 1.69-1.50 (m, 5H), 1.45- |
| | | | | 1.30 (m, 1H), 1.36-1.21 (m, 1H), 1.20-1.06 (m, 2H), |
| | | | | 1.03 (s, 3H), 0.91-0.65 (m, 2H) |

^{a}The coupling could be performed with a variety of bases including TEA and DIEA in DCM or DMF. The reaction was run anywhere from 25-50° C. for 0.5-3 hrs. The final compounds were purified via standard techniques including prep-HPLC and various chromatography techniques.
^{b}The product of the coupling was further deprotected with HCl in dioxane or MeOH for 0.5-2 hr at 35-40° C. The final cmpd was purified by prep-HPLC.

### Example 3. Preparation of Compounds of the Invention (Method 4)

### Synthesis of 3-[4-[4-[2-[4-[4-[(7′-Cyclopentyl-6′-oxo-spiro[cyclopropane-1,5′-pyrrolo[2,3-d]pyrimidine]-2′-yl)amino]-3-methyl-phenyl]sulfonyl-1-piperidyl]ethyl]piperazin-1-yl]-3-methyl-2-oxo-benzimidazol-1-yl]piperidine-2,6-dione (I-50)

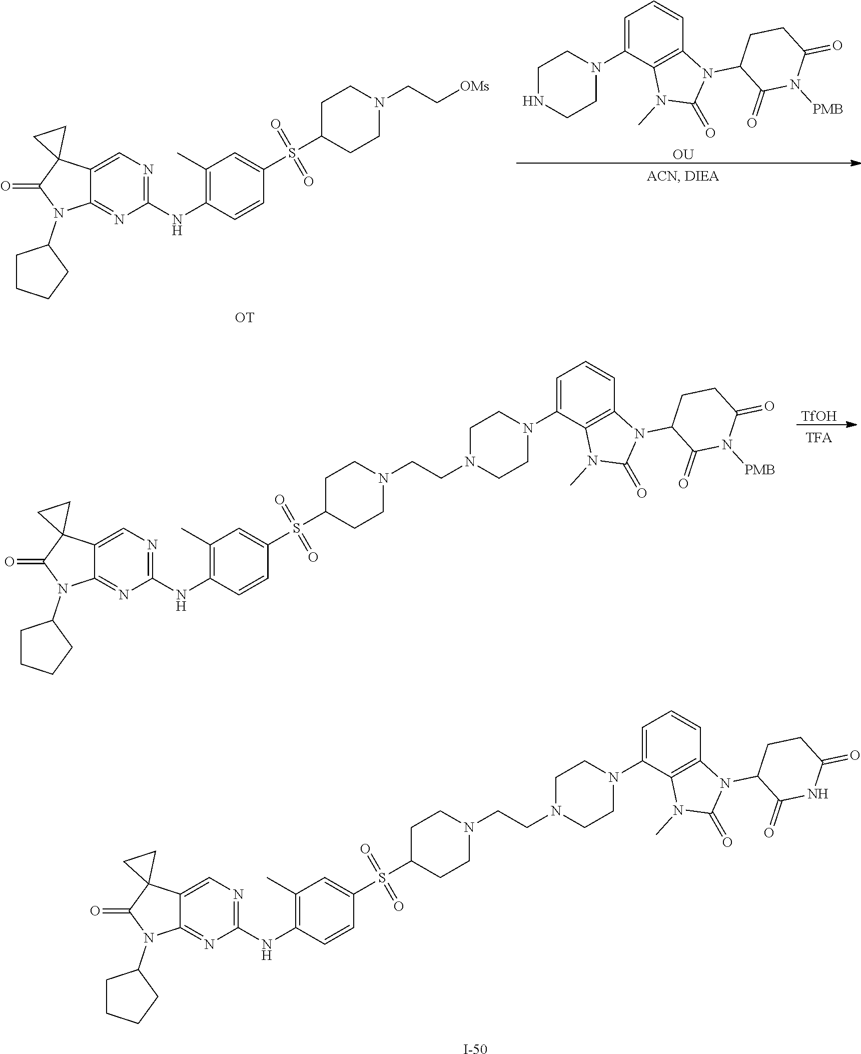

### Step 1—3-[4-[4-[2-[4-[4-[(7′-Cyclopentyl-6′-oxo-spiro[cyclopropane-1,5′-pyrrolo[2,3-d]pyrimidine]-2′-yl)amino]-3-methyl-phenyl]sulfonyl-1-piperidyl]ethyl]piperazin-1-yl]-3-methyl-2-oxo-benzimidazol-1-yl]-1-[(4-methoxyphenyl)methyl]piperidine-2,6-dione

To a solution of 1-[(4-methoxyphenyl)methyl]-3-(3-methyl-2-oxo-4-piperazin-1-yl-benzimidazol-1-yl) piperidine-2,6-dione (105 mg, 182 umol, TFA, Intermediate OU) in ACN (2 mL) was added DIEA (95.2 uL, 546 umol). Then 2-[4-[4-[(7′-cyclopentyl-6′-oxo-spiro[cyclopropane-1,5′-pyrrolo [2,3-d]pyrimidine]-2′-yl)amino]-3-methyl-phenyl]sulfonyl-1-piperidyl]ethyl methanesulfonate (110 mg, 182 umol, Intermediate OT) was added, and the mixture was stirred at 50° C. for 10 hrs. On completion, the mixture was filtered and the filtrate was concentrated in vacuo to give the residue. The residue was purified by prep-HPLC (column: Phenomenex luna C18 150*25 mm*10 um; mobile phase: [water (FA)-ACN]; B %: 25%-55%, 8 min) to give the title compound (25.0 mg, 14% yield) as white solid. ¹H NMR (400 MHz, DMSO-d₆) δ 8.94 (s, 1H), 8.01-7.95 (m, 2H), 7.66 (d, J=1.6 Hz, 1H), 7.63-7.58 (m, 1H), 7.20 (d, J=8.8 Hz, 2H), 6.94-6.89 (m, 2H), 6.87-6.82 (m, 2H), 6.81-6.73 (m, 1H), 5.52-5.47 (m, 1H), 4.84-4.70 (m, 3H), 3.72 (s, 3H), 3.61 (s, 3H), 3.08-2.92 (m, 8H), 2.80-2.71 (m, 4H), 2.37 (s, 4H), 2.15-2.00 (m, 6H), 1.94-1.73 (m, 9H), 1.71-1.67 (m, 3H), 1.56-1.48 (m, 6H). LC-MS (ESI⁺) m/z 971.4 (M+H)⁺.

### Step 2—3-[4-[4-[2-[4-[4-[(7′-Cyclopentyl-6′-oxo-spiro[cyclopropane-1,5′-pyrrolo[2,3-d]pyrimidine]-2′-yl)amino]-3-methyl-phenyl]sulfonyl-1-piperidyl]ethyl]piperazin-1-yl]-3-methyl-2-oxo-benzimidazol-1-yl]piperidine-2,6-dione

A solution of 3-[4-[4-[2-[4-[4-[(7′-cyclopentyl-6′-oxo-spiro[cyclopropane-1,5′-pyrrolo[2,3-d]pyrimidine]-2′-yl)amino]-3-methyl-phenyl]sulfonyl-1-piperidyl]ethyl]piperazin-1-yl]-3-methyl-2-oxo-benzimidazol-1-yl]-1-[(4-methoxyphenyl)methyl]piperidine-2,6-dione (20.0 mg, 20.6 umol) in TFA (1 mL) and TfOH (0.3 mL) was stirred at 70° C. for 1 hr. On completion, the mixture was concentrated in vacuo to give the residue. The residue was diluted with ACN (1 mL) and then TEA was added to adjust pH=2-3. The residue was purified by prep-HPLC (column: Welch Ultimate C18 150*25 mm*5 um; mobile phase: [water (NH₄HCO₃)-ACN]; B %: 34%-64%, 10 min) to give the title compound (4.45 mg, 25% yield) as white solid. ¹H NMR (400 MHz, DMSO-d₆) δ 11.08 (s, 1H), 8.92 (s, 1H), 7.96 (t, J=4.4 Hz, 2H), 7.65 (s, 1H), 7.60 (d, J=8.0 Hz, 1H), 6.99-6.93 (m, 1H), 6.92-6.85 (m, 2H), 5.35-5.31 (m, 1H), 4.74-4.69 (m, 1H), 3.60 (s, 3H), 3.18-3.09 (m, 2H), 2.99-2.83 (m, 8H), 2.69-2.65 (m, 2H), 2.43 (s, 4H), 2.36 (s, 3H), 2.11-2.04 (m, 2H), 1.97 (s, 2H), 1.96-1.89 (m, 2H), 1.84-1.67 (m, 9H), 1.57-1.47 (m, 6H). LC-MS (ESI⁺) m z 851.3 (M+H)⁺.

### Example 4. Preparation of Compounds of the Invention (Method 7)

Synthesis of 3-[5-[1-[[4-[3-[4-[(6-Chloro-8-cyclopentyl-7-oxo-pyrido[2,3-d]pyrimidin-2-yl)amino]-3-methyl-phenyl]sulfonylpropoxy]cyclohexyl]methyl]-4-piperidyl]-3-methyl-2-oxo-benzimidazol-1-yl]piperidine-2,6-dione (I-3)

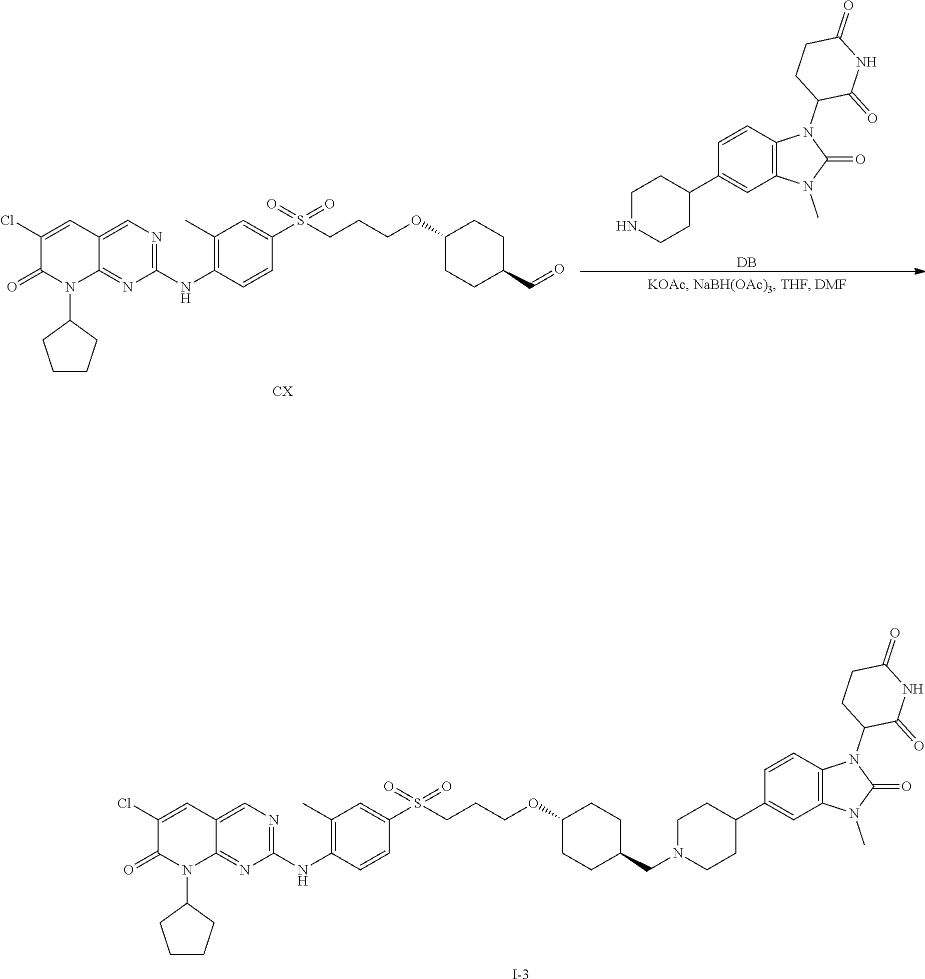

To a solution of 4-[3-[4-[(6-chloro-8-cyclopentyl-7-oxo-pyrido[2,3-d]pyrimidin-2-yl)amino]-3-methyl-phenyl]sulfonylpropoxy]cyclohexanecarbaldehyde (31.0 mg, 52.8 umol, Intermediate CX) in THF (1 mL) was added KOAc (51.8 mg, 527 umol) and 3-[3-methyl-2-oxo-5-(4-piperidyl)benzimidazol-1-yl]piperidine-2,6-dione (19.8 mg, 58.0 umol, Intermediate DB) at 0° C. After 30 minutes, NaBH(OAc)₃ (5.60 mg, 26.4 umol) was added dropwise at 0° C. and the mixture was stirred at 0° C. for 2 hrs. On completion, the mixture was filtered and concentrated in vacuo to give a residue. The residue was purified by prep-HPLC (column: Phenomenex luna C18 150*25 mm*10 um; mobile phase: [water (FA)-ACN]; B %: 22%-52%, 15 min) to give the title compound (8.58 mg, 17% yield, FA salt) as a white solid. ¹H NMR (400 MHz, DMSO-d₆) δ 11.09 (s, 1H), 9.74 (s, 1H), 8.77 (s, 1H), 8.19 (s, 1H), 7.85-7.78 (m, 2H), 7.72 (d, J=8.4 Hz, 1H), 7.09 (s, 1H), 6.99 (d, J=8.4 Hz, 1H), 6.89 (d, J=8.0 Hz, 1H), 5.80-5.69 (m, 1H), 5.33 (dd, J=5.2, 12.8 Hz, 1H), 3.32 (s, 3H), 3.30 (s, 2H), 3.12-2.99 (m, 2H), 2.88 (d, J=10.0 Hz, 2H), 2.72-2.61 (m, 4H), 2.37 (s, 3H), 2.12-2.07 (m, 2H), 2.04 (d, J=7.2 Hz, 2H), 1.91-1.87 (m, 3H), 1.79-1.64 (m, 14H), 1.53-1.37 (m, 4H), 1.10-1.00 (m, 2H), 0.86-0.75 (m, 2H). LC-MS (ESI⁺) m/z 913.8 (M+H)⁺.

TABLE 3
Compounds synthesized via Method 7, the reductive amination
of the corresponding amines and aldehyde/ketones.

| | | | LCMS | |
|---|---|---|---|---|
| | | | (ESI+) | |
| | | | m/z | |
| I-#^{a} | Amine | Aldehyde | (M+H)+ | ¹H NMR (400 MHz, DMSO-d₆) δ |
| I-1 | EC | DM | 892.4 | 11.05 (s, 1H), 9.69 (s, 1H), 8.74 (s, 1H), 8.22 (s, 1H), 8.17 |
| | | | | (s, 1H), 7.97-7.91 (m, 2H), 7.78-7.72 (m, 2H), 6.94-6.88 |
| | | | | (m, 1H), 6.79 (d, J = 2.0 Hz, 1H), 6.64-6.58 (m, 1H), 5.32- |
| | | | | 5.24 (m, 1H), 3.93 (s, 3H), 3.58-3.49 (m, 2H), 3.29 (s, |
| | | | | 3H), 2.93-2.84 (m, 1H), 2.69-2.61 (m, 2H), 2.57 (d, J = |
| | | | | 12.4 Hz, 3H), 2.43 (d, J = 6.4 Hz, 2H), 2.38 (s, 3H), 2.31- |
| | | | | 2.17 (m, 8H), 2.10-2.04 (m, 2H), 2.03-1.94 (m, 1H), 1.75- |
| | | | | 1.66 (m, 2H), 1.62-1.41 (m, 5H), 1.26-1.11 (m, 2H) |
| I-2 | DK | DM | 913.5 | 11.0 (s, 1H), 9.70 (s, 1H), 8.83-8.68 (m, 1H), 8.20-8.15 |
| | | | | (m, 1H), 7.84-7.82 (m, 1H), 7.71-7.68 (m, 1H), 6.95- |
| | | | | 6.87 (m, 1H), 6.81-6.74 (m, 1H), 6.64-6.54 (m, 1H), 5.83- |
| | | | | 5.68 (m, 1H), 5.35-5.21 (m, 1H), 3.57-3.48 (m, 3H), |
| | | | | 3.29-3.20 (m, 3H), 2.65-2.56 (m, 4H), 2.38-2.09 (m, |
| | | | | 17H), 2.00-1.96 (m, 2H), 1.71 (s, 6H), 1.60-1.43 (m, 7H), |
| | | | | 1.27-1.10 (m, 4H) |
| I-3 | DB | CX | 913.8 | 11.09 (s, 1H), 9.74 (s, 1H), 8.77 (s, 1H), 8.19 (s, 1H), 7.85- |
| | | | | 7.78 (m, 2H), 7.72 (d, J = 8.4 Hz, 1H), 7.09 (s, 1H), 6.99 (d, |
| | | | | J = 8.4 Hz, 1H), 6.89 (d, J = 8.0 Hz, 1H), 5.80-5.69 (m, |
| | | | | 1H), 5.33 (dd, J = 5.2, 12.8 Hz, 1H), 3.32 (s, 3H), 3.30 (s, |
| | | | | 2H), 3.12-2.99 (m, 2H), 2.88 (d, J = 10.0 Hz, 2H), 2.72- |
| | | | | 2.61 (m, 4H), 2.37 (s, 3H), 2.12-2.07 (m, 2H), 2.04 (d, J = |
| | | | | 7.2 Hz, 2H), 1.91-1.87 (m, 3H), 1.79-1.64 (m, 14H), 1.53- |
| | | | | 1.37 (m, 4H), 1.10-1.00 (m, 2H), 0.86-0.75 (m, 2H) |
| I-4 | DB | KU | 866.4 | 11.07 (s, 1H), 9.06 (s, 1H), 8.39 (d, J = 5.2 Hz, 1H), 7.80 (d, |
| | | | | J = 8.4 Hz, 1H), 7.76 (d, J = 1.6 Hz, 1H), 7.68 (dd, J = 2.0, |
| | | | | 8.4 Hz, 1H), 7.46 (s, 1H), 7.12-7.08 (m, 2H), 7.01-6.97 |
| | | | | (m, 1H), 6.90 (d, J = 8.4 Hz, 1H), 5.53-5.44 (m, 1H), 5.33 |
| | | | | (dd, J = 5.2, 12.4 Hz, 1H), 3.43 (t, J = 6.4 Hz, 2H), 3.32- |
| | | | | 3.32 (m, 3H), 3.29-3.24 (m, 2H), 3.06 (s, 1H), 2.95-2.83 |
| | | | | (m, 3H), 2.70 (s, 1H), 2.64-2.58 (m, 2H), 2.44 (s, 3H), 2.35- |
| | | | | 2.33 (m, 3H), 2.08-1.96 (m, 3H), 1.95-1.86 (m, 4H), |
| | | | | 1.80-1.65 (m, 8H), 1.47-1.36 (m, 1H), 1.26 (d, J = 7.2 Hz, |
| | | | | 6H), 1.12-1.00 (m, 2H), 0.87-0.76 (m, 2H) |
| I-6 | DB | KR | 903.3 | 11.09 (s, 1H), 9.86 (s, 1H), 8.90 (s, 1H), 8.17 (s, 1H), 7.88- |
| | | | | 7.77 (m, 2H), 7.76-7.70 (m, 1H), 7.11-6.71 (m, 4H), 5.66- |
| | | | | 5.49 (m, 1H), 5.35 (dd, J = 5.2, 12.8 Hz, 1H), 3.44 (s, 3H), |
| | | | | 3.22-3.05 (m, 3H), 2.99-2.84 (m, 3H), 2.73-2.62 (m, |
| | | | | 6H), 2.37 (s, 3H), 2.34-2.28 (m, 1H), 2.07 (s, 1H), 2.04- |
| | | | | 1.90 (m, 6H), 1.84-1.68 (m, 6H), 1.39 (d, J = 5.2 Hz, 6H), |
| | | | | 1.24 (s, 1H), 1.16-1.06 (m, 2H), 1.04-0.86 (m, 2H) |
| I-10^{b} | KY | KW | 913.3 | 11.09 (s, 1H), 9.74 (s, 1H), 8.77 (s, 1H), 8.20 (s, 1H), 7.77- |
| | | | | 7.68 (m, 2H), 7.03-6.96 (m, 1H), 6.89 (br d, J = 8.4 Hz, |
| | | | | 2H), 5.40-5.31 (m, 1H), 3.76-3.53 (m, 9H), 3.20-3.02 |
| | | | | (m, 8H), 2.77-2.69 (m, 3H), 2.41 (br s, 3H), 2.25-2.11 (m, |
| | | | | 5H), 2.04-1.77 (m, 10H), 1.52-1.32 (m, 9H) |
| I-11 | KX | LA | 844.5 | 11.08 (s, 1H), 9.73 (s, 1H), 8.76 (s, 1H), 8.18-8.13 (m, 1H), |
| | | | | 7.87-7.80 (m, 1H), 7.74 (s, 1H), 7.68 (dd, J = 1.6, 8.8 Hz, |
| | | | | 1H), 6.98-6.93 (m, 1H), 6.90-6.83 (m, 2H), 5.67-5.54 |
| | | | | (m, 1H), 5.36-5.31 (m, 1H), 3.60 (s, 3H), 3.24-3.15 (m, |
| | | | | 2H), 3.07 (d, J = 10.4 Hz, 2H), 2.96 (d, J = 10.8 Hz, 2H), |
| | | | | 2.70-2.63 (m, 1H), 2.72-2.61 (m, 4H), 2.36 (s, 3H), 2.33 |
| | | | | (s, 2H), 2.01-1.95 (m, 1H), 1.93-1.83 (m, 4H), 1.74 (d, J = |
| | | | | 8.8 Hz, 2H), 1.52-1.47 (m, 2H), 1.43-1.34 (m, 10H) |
| I-12 | DB | LJ | 927.4 | 11.08 (s, 1H), 9.73 (s, 1H), 8.78 (s, 1H), 8.19 (s, 1H), 7.85 |
| | | | | (d, J = 8.4 Hz, 1H), 7.75 (d, J = 1.6 Hz, 1H), 7.68 (dd, J = |
| | | | | 2.0, 8.4 Hz, 1H), 7.08 (s, 1H), 6.99 (d, J = 8.4 Hz, 1H), 6.89 |
| | | | | (d, J = 7.8 Hz, 1H), 5.75 (t, J = 8.4 Hz, 1H), 5.33 (dd, J = |
| | | | | 5.6, 12.8 Hz, 1H), 3.53-3.48 (m, 2H), 3.47-3.42 (m, 4H), |
| | | | | 3.33 (s, 3H), 3.09-3.03 (m, 1H), 2.88 (d, J = 10.4 Hz, 3H), |
| | | | | 2.75-2.67 (m, 1H), 2.61 (d, J = 17.6 Hz, 1H), 2.48-2.43 |
| | | | | (m, 1H), 2.37 (s, 3H), 2.11 (d, J = 8.4 Hz, 2H), 2.05 (d, J = |
| | | | | 7.2 Hz, 2H), 2.01-1.96 (m, 1H), 1.95-1.86 (m, 4H), 1.71 |
| | | | | (s, 8H), 1.51-1.39 (m, 4H), 1.20 (d, J = 6.8 Hz, 3H), 1.12- |
| | | | | 0.99 (m, 2H), 0.80 (g, J = 12.4 Hz, 2H) |
| I-14 | KT | DM | 899.2 | 11.05 (s, 1H), 9.70 (s, 1H), 8.76 (s, 1H), 8.18 (s, 1H), 7.86 |
| | | | | (d, J = 8.0 Hz, 1H), 7.76 (s, 1H), 7.70 (d, J = 8.4 Hz, 1H), |
| | | | | 6.91 (d, J = 8.6 Hz, 1H), 6.80 (d, J = 1.2 Hz, 1H), 6.61 (d, J = |
| | | | | 8.6 Hz, 1H), 5.64 (d, J = 5.6 Hz, 1H), 5.28 (dd, J = 5.4, |
| | | | | 12.8 Hz, 1H), 3.57-3.51 (m, 4H), 3.29 (s, 3H), 3.22 (t, J = |
| | | | | 6.8 Hz, 2H), 2.95-2.83 (m, 1H), 2.73-2.63 (m, 6H), 2.60 |
| | | | | (d, J = 13.2 Hz, 3H), 2.37 (s, 3H), 2.11 (d, J = 6.8 Hz, 2H), |
| | | | | 2.01-1.95 (m, 1H), 1.92-1.83 (m, 3H), 1.77-1.72 (m, |
| | | | | 2H), 1.61-1.55 (m, 1H), 1.50 ( d, J = 11.2 Hz, 2H), 1.44 (d, |
| | | | | J = 6.8 Hz, 6H), 1.24-1.18 (m, 2H), 1.11-1.03 (m, 2H) |
| I-38^{b} | PG | MU | 914.3 | 11.08 (br s, 1H), 9.71 (s, 1H), 8.76 (s, 1H), 8.18 (s, 1H), 7.78 |
| | | | | (d, J = 8.4 Hz, 1H), 7.73 (s, 1H), 7.69-7.64 (m, 1H), 6.83 |
| | | | | (br d, J = 8.5 Hz, 1H), 6.70 (br t, J = 8.1 Hz, 1H), 5.77-5.68 |
| | | | | (m, 1H), 5.31 (br dd, J = 5.1, 12.6 Hz, 1H), 4.12-4.04 (m, |
| | | | | 1H), 3.45 (s, 3H), 2.92-2.85 (m, 1H), 2.68-2.56 (m, 6H), |
| | | | | 2.34 (s, 3H), 2.14-2.05 (m, 7H), 2.01-1.87 (m, 4H), 1.76- |
| | | | | 1.67 (m, 6H), 1.58-1.41 (m, 8H), 1.28-1.20 (m, 2H) |
| I-42 | OY | MV | 876.2 | 11.08 (s, 1H), 8.90 (s, 1H), 7.97-7.90 (m, 2H), 7.66 (d, J = |
| | | | | 2.0 Hz, 1H), 7.60 (dd, J = 1.9, 8.4 Hz, 1H), 6.99-6.93 (m, |
| | | | | 1H), 6.90-6.82 (m, 2H), 5.34 (dd, J = 5.2, 12.6 Hz, 1H), |
| | | | | 4.72 (quin, J = 8.4 Hz, 1H), 4.05 (quin, J = 8.4 Hz, 1H), 3.60 |
| | | | | (s, 3H), 3.09 (d, J = 9.6 Hz, 2H), 2.95-2.81 (m, 1H), 2.70- |
| | | | | 2.56 (m, 4H), 2.35 (s, 3H), 2.21 (dd, J = 2.4, 9.6 Hz, 3H), |
| | | | | 2.16-2.01 (m, 7H), 2.00-1.95 (m, 1H), 1.95-1.87 (m, |
| | | | | 2H), 1.84-1.73 (m, 6H), 1.70-1.67 (m, 2H), 1.53 (dt, J = |
| | | | | 4.4, 7.6 Hz, 9H), 1.32-1.22 (m, 2H) |
| I-44 | PB | MV | 922.4 | 11.08 (s, 1H), 9.69 (s, 1H), 8.75 (s, 1H), 8.18 (s, 1H), 7.85- |
| | | | | 7.62 (m, 3H), 7.01-6.93 (m, 1H), 6.90-6.83 (m, 2H), 5.78- |
| | | | | 5.50 (m, 1H), 5.34 (dd, J = 5.2, 12.8 Hz, 1H), 4.11 (t, J = |
| | | | | 8.8 Hz, 1H), 3.61 (s, 3H), 3.14-3.06 (m, 2H), 2.97-2.79 |
| | | | | (m, 2H), 2.74-2.62 (m, 5H), 2.54 (s, 2H), 2.50 (s, 3H), 2.34 |
| | | | | (s, 4H), 2.14-2.07 (m, 2H), 1.98 (dd, J = 5.2, 10.0 Hz, 5H), |
| | | | | 1.78 (d, J = 11.6 Hz, 2H), 1.73-1.53 (m, 6H), 1.38-1.24 |
| | | | | (m, 3H), 0.60-0.24 (m, 3H), 0.13-0.01 (m, 1H) |
| I-51 | OS | OX | 850.4 | 11.06 (br d, J = 2.4 Hz, 1H), 8.92 (s, 1H), 8.00-7.93 (m, |
| | | | | 2H), 7.66 (d, J = 1.6 Hz, 1H), 7.62-7.56 (m, 1H), 6.98- |
| | | | | 6.92 (m, 1H), 6.91-6.82 (m, 2H), 5.36-5.30 (m, 1H), 4.78- |
| | | | | 4.66 (m, 1H), 3.60 (s, 3H), 3.17-3.12 (m, 1H), 3.09-3.03 |
| | | | | (m, 2H), 2.93 (br d, J = 11.2 Hz, 2H), 2.89-2.83 (m, 1H), |
| | | | | 2.73-2.65 (m, 2H), 2.59 (br d, J = 2.0 Hz, 1H), 2.37 (s, 3H), |
| | | | | 2.34-2.30 (m, 1H), 2.11-2.04 (m, 2H), 2.03-1.95 (m, 2H), |
| | | | | 1.89-1.80 (m, 6H), 1.77-1.71 (m, 4H), 1.70-1.67 (m, |
| | | | | 2H), 1.54-1.47 (m, 6H), 1.37 (br d, J = 7.2 Hz, 4H), 1.23 |
| | | | | (br s, 2H) |
| I-53 | EG | PA | 864.4 | 11.07 (s, 1H), 8.92 (s, 1H), 8.00-7.87 (m, 2H), 7.64 (s, 1H), |
| | | | | 7.58 (dd, J = 2.0, 8.4 Hz, 1H), 6.98-6.92 (m, 1H), 6.90- |
| | | | | 6.79 (m, 2H), 5.33 (dd, J = 5.6, 12.8 Hz, 1H), 4.79-4.64 |
| | | | | (m, 1H), 3.60 (s, 3H), 3.11 (d, J = 10.0 Hz, 3H), 2.91-2.83 |
| | | | | (m, 1H), 2.72-2.58 (m, 5H), 2.38 (d, J = 1.6 Hz, 1H), 2.36 |
| | | | | (s, 3H), 2.17 (s, 3H), 2.11-1.91 (m, 6H), 1.89-1.78 (m, |
| | | | | 4H), 1.75-1.66 (m, 6H), 1.63-1.56 (m, 2H), 1.54-1.47 |
| | | | | (m, 4H), 1.39-1.22 (m, 3H), 0.94-0.75 (m, 2H) |
| I-54 | LG | LA | 870.1 | 11.08 (s, 1H), 9.75 (s, 1H), 8.78 (s, 1H), 8.22-8.15 (m, 1H), |
| | | | | 7.85-7.79 (m, 1H), 7.77-7.72 (m, 1H), 7.67 (d, J = 1.6, |
| | | | | 8.4 Hz, 1H), 7.00-6.93 (m, 1H), 6.91-6.82 (m, 2H), 5.81- |
| | | | | 5.68 (m, 1H), 5.39-5.29 (m, 1H), 3.60 (s, 3H), 3.06 (d, J = |
| | | | | 10.4 Hz, 2H), 2.97-2.91 (m, 2H), 2.68-2.65 (m, 2H), |
| | | | | 2.63 (d, J = 4.4 Hz, 2H), 2.62-2.56 (m, 2H), 2.37 (s, 3H), |
| | | | | 2.34-2.28 (m, 2H), 2.15-2.06 (m, 2H), 1.99 (d, J = 4.4, |
| | | | | 10.8 Hz, 2H), 1.91-1.82 (m, 4H), 1.77-1.67 (m, 6H), 1.54- |
| | | | | 1.44 (m, 4H), 1.41-1.33 (m, 4H) |
| I-55 | LA | LI | 849.3 | 11.08 (s, 1H), 9.69 (s, 1H), 8.74 (s, 1H), 8.23 (s, 1H), 8.00- |
| | | | | 7.90 (m, 2H), 7.75-7.66 (m, 2H), 6.94 (d, J = 8.0 Hz, 1H), |
| | | | | 6.90-6.82 (m, 2H), 5.37-5.30 (m, 1H), 3.93 (s, 3H), 3.60 |
| | | | | (s, 3H), 3.21 (d, J = 13.6 Hz, 2H), 3.06 (d, J = 9.6 Hz, 2H), |
| | | | | 2.96-2.92 (m, 2H), 2.68-2.63 (m, 3H), 2.39 (s, 3H), 2.30 |
| | | | | (s, 2H), 2.01-1.97 (m, 1H), 1.91-1.85 (m, 2H), 1.84 (s, |
| | | | | 2H), 1.76-1.71 (m, 2H), 1.52 (dd, J = 2.0, 11.6 Hz, 2H), |
| | | | | 1.43-1.29 (m, 6H) |
| I-58 | PG | MV | 896.1 | 11.08 (s, 1H), 9.72 (s, 1H), 8.89-8.74 (m, 2H), 8.19 (s, 1H), |
| | | | | 7.82 (d, J = 8.4 Hz, 1H), 7.76 (d, J = 1.6 Hz, 1H), 7.68 (dd, |
| | | | | J = 1.6, 8.4 Hz, 1H), 7.04-6.95 (m, 1H), 6.93-6.84 (m, |
| | | | | 2H), 5.84-5.68 (m, 1H), 5.42-5.27 (m, 1H), 4.20-4.07 |
| | | | | (m, 1H), 3.62 (s, 3H), 3.18-3.09 (m, 2H), 3.07-2.98 (m, |
| | | | | 2H), 2.96-2.83 (m, 3H), 2.75-2.58 (m, 4H), 2.36 (s, 3H), |
| | | | | 2.29-2.05 (m, 6H), 2.04-1.89 (m, 4H), 1.87-1.68 (m, |
| | | | | 9H), 1.55-1.36 (m, 4H) |
| I-59 | OW | OX | 896 | 11.23-10.95 (m, 1H), 9.71 (s, 1H), 8.76 (s, 1H), 8.18 (s, |
| | | | | 1H), 7.82 (dd, J = 3.2, 8.0 Hz, 1H), 7.74 (s, 1H), 7.70-7.63 |
| | | | | (m, 1H), 7.00-6.92 (m, 1H), 6.91-6.82 (m, 2H), 5.85- |
| | | | | 5.57 (m, 1H), 5.38-5.30 (m, 1H), 3.60 (s, 3H), 3.10-3.04 |
| | | | | (m, 2H), 2.95-2.88 (m, 2H), 2.64-2.55 (m, 4H), 2.35 (s, |
| | | | | 3H), 2.31-2.27 (m, 2H), 2.05-1.67 (m, 12H), 1.56-1.49 |
| | | | | (m, 2H), 1.42-1.22 (m, 8H), 1.18 (d, J = 6.4 Hz, 2H), 0.50- |
| | | | | 0.32 (m, 2H) |
| I-68 | DB | KV | 892.3 | 11.32-10.76 (m, 1H), 9.83-9.57 (m, 1H), 8.74 (s, 1H), |
| | | | | 8.23 (s, 1H), 7.98-7.90 (m, 2H), 7.80-7.71 (m, 2H), 7.08 |
| | | | | (s, 1H), 6.99 (d, J = 8.0 Hz, 1H), 6.90 (dd, J = 0.8, 8.0 Hz, |
| | | | | 1H), 5.35-5.29 (m, 1H), 3.93 (s, 3H), 3.41 (t, J = 6.0 Hz, |
| | | | | 2H), 3.28 (s, 3H), 3.10-2.96 (m, 2H), 2.89-2.85 (m, 2H), |
| | | | | 2.76-2.68 (m, 1H), 2.65-2.57 (m, 2H), 2.38 (s, 3H), 2.03 |
| | | | | (d, J = 7.2 Hz, 2H), 2.01-1.96 (m, 1H), 1.93 (d, J = 2.8 Hz, |
| | | | | 1H), 1.91-1.83 (m, 4H), 1.75 (d, J = 6.0 Hz, 3H), 1.72- |
| | | | | 1.65 (m, 5H), 1.42-1.37 (m, 1H), 1.23 (s, 1H), 1.09-1.00 |
| | | | | (m, 2H), 0.83-0.73 (m, 2H) |
| I-119 | MY | MV | 907.3 | 11.08 (s, 1H), 9.68 (s, 1H), 8.63 (s, 1H), 7.89 (d, J = 8.4 Hz, |
| | | | | 1H), 7.71 (s, 1H), 7.63 (d, J = 8.4 Hz, 1H), 7.00-6.93 (m, |
| | | | | 1H), 6.90-6.82 (m, 2H), 5.37-5.28 (m, 1H), 4.14-4.06 |
| | | | | (m, 1H), 4.02 (s, 1H), 3.60 (s, 3H), 3.12-3.06 (m, 2H), 2.93- |
| | | | | 2.84 (m, 1H), 2.69 (s, 2H), 2.68-2.61 (m, 3H), 2.36 (s, |
| | | | | 3H), 2.29-2.19 (m, 3H), 2.17-2.04 (m, 6H), 2.01-1.90 |
| | | | | (m, 3H), 1.77 (d, J = 12.4 Hz, 2H), 1.67-1.43 (m, 10H), |
| | | | | 1.40-1.32 (m, 2H), 1.24 (d, J = 5.6 Hz, 2H), 1.13 (s, 3H) |
| I-125 | PE | MZ | 897.4 | 11.11 (s, 1H), 8.87 (s, 1H), 8.10 (s, 1H), 7.87 (d, J = 8.4 Hz, |
| | | | | 1H), 7.67 (d, J = 1.6 Hz, 1H), 7.63-7.59 (m, 1H), 6.84 (d, |
| | | | | J = 8.8 Hz, 1H), 6.74-6.70 (m, 1H), 5.33 (dd, J = 5.2, 12.4 |
| | | | | Hz, 1H), 5.11-5.06 (m, 1H), 4.27 (s, 2H), 4.09-4.04 (m, |
| | | | | 1H), 3.47 (d, J = 1.8 Hz, 4H), 3.20 (d, J = 2.4 Hz, 4H), 2.90 |
| | | | | (s, 3H), 2.64 (s, 2H), 2.34 (s, 3H), 2.06 (dd, J = 8.4, 11.2 Hz, |
| | | | | 6H), 1.94-1.88 (m, 3H), 1.80-1.73 (m, 4H), 1.71-1.65 |
| | | | | (m, 5H), 1.57-1.50 (m, 5H), 1.47-1.40 (m, 3H), 1.30- |
| | | | | 1.22 (m, 3H) |
| I-162 | NA | MZ | 880.8 | 11.10 (s, 1H), 9.58 (s, 1H), 8.76 (s, 1H), 7.82-7.75 (m, 2H), |
| | | | | 7.74 (d, J = 1.6 Hz, 1H), 7.69-7.63 (m, 1H), 6.84 (d, J = |
| | | | | 8.4 Hz, 1H), 6.71 (t, J = 8.0 Hz, 1H), 6.35 (d, J = 9.2 Hz, |
| | | | | 1H), 5.73-5.62 (m, 1H), 5.36-5.27 (m, 1H), 4.16-4.03 |
| | | | | (m, 1H), 3.49-3.45 (m, 4H), 3.20 (d, J = 11.6 Hz, 2H), 2.91- |
| | | | | 2.84 (m, 1H), 2.65-2.58 (m, 4H), 2.35 (s, 3H), 2.26-2.15 |
| | | | | (m, 4H), 2.14-2.04 (m, 6H), 2.02-1.97 (m, 1H), 1.95- |
| | | | | 1.88 (m, 2H), 1.75 (d, J = 10.4 Hz, 2H), 1.70-1.62 (m, 4H), |
| | | | | 1.56 (s, 2H), 1.52 (d, J = 4.8 Hz, 2H), 1.46 (d, J = 7.2 Hz, |
| | | | | 2H), 1.28-1.20 (m, 2H) |
| I-175 | QA | MU | 882.3 | 11.09 (s, 1H), 8.85 (s, 1H), 8.17 (s, 1H), 7.87 (d, J = 8.4 Hz, |
| | | | | 1H), 7.66 (s, 1H), 7.60 (dd, J = 1.6, 8.4 Hz, 1H), 6.83 (d, J = |
| | | | | 8.4 Hz, 1H), 6.71 (t, J = 8.0 Hz, 1H), 5.32 (dd, J = 5.2, |
| | | | | 12.8 Hz, 1H), 5.19 (t, J = 8.4 Hz, 1H), 4.05 (br t, J = 8.8 Hz, |
| | | | | 1H), 3.50-3.44 (m, 6H), 2.91-2.85 (m, 1H), 2.75-2.68 |
| | | | | (m, 3H), 2.34 (s, 3H), 2.27-2.15 (m, 4H), 2.13-1.94 (m, |
| | | | | 9H), 1.92 (d, J = 9.6 Hz, 2H), 1.78-1.63 (m, 7H), 1.60- |
| | | | | 1.39 (m, 8H), 1.29-1.20 (m, 2H) |
| I-183 | PG | PC | 883.5 | 11.19 (s, 1H), 9.73 (s, 1H), 8.77 (s, 1H), 8.19 (s, 1H), 7.79 |
| | | | | (d, J = 8.4 Hz, 1H), 7.74 (s, 1H), 7.67 (d, J = 2.0, 8.4 Hz, |
| | | | | 1H), 7.10-7.04 (m, 1H), 6.75 (d, J = 7.6 Hz, 1H), 6.69 (d, |
| | | | | J = 8.1 Hz, 1H), 5.80-5.67 (m, 1H), 5.31 (d, J = 5.2, 12.8 |
| | | | | Hz, 1H), 4.10 (t, J = 8.4 Hz, 1H), 3.66-3.62 (m, 2H), 2.88- |
| | | | | 2.82 (m, 1H), 2.77-2.64 (m, 5H), 2.35 (s, 3H), 2.27-2.18 |
| | | | | (m, 3H), 2.14-2.04 (m, 7H), 1.95-1.88 (m, 2H), 1.80- |
| | | | | 1.66 (m, 7H), 1.58-1.44 (m, 6H), 1.25-1.18 (m, 2H) |
| I-191 | QB | MV | 908.3 | 1.15 (s, 3H), 1.31-1.40 (m, 2H), 1.51 (t, J = 4.2 Hz, 4H), |
| | | | | 1.63-1.74 (m, 4H), 1.75-1.83 (m, 3H), 1.93-2.04 (m, |
| | | | | 4H), 2.08-2.15 (m, 2H), 2.35 (s, 3H), 2.57-2.75 (m, 9H), |
| | | | | 2.83-2.93 (m, 2H), 3.07-3.16 (m, 3H), 3.61 (s, 3H), 3.73 |
| | | | | (d, J = 13.2 Hz, 2H), 4.0-4.16 (m, 1H), 4.44 (s, 1H), 5.34 |
| | | | | (m, 1H), 6.85-6.91 (m, 2H), 6.94-7.01 (m, 1H), 7.56- |
| | | | | 7.70 (m, 2H), 7.93 (d, J = 8.4 Hz, 1H), 8.38 (s, 1H), 9.15 (s, |
| | | | | 1H), 11.08 (s, 1H) |
| I-272 | NA | QC | 809.4 | 10.91 (s, 1H), 9.58 (s, 1H), 8.76 (s, 1H), 7.81-7.76 (m, 2H), |
| | | | | 7.74 (d, J = 1.6 Hz, 1H), 7.67-7.65 (m, 1H), 7.32 (d, J = 8.0 |
| | | | | Hz, 1H), 6.35 (d, J = 9.4 Hz, 1H), 6.13-6.10(m, 1H), 5.73- |
| | | | | 5.64 (m, 1H), 5.44 (d, J = 2.4 Hz, 1H), 4.09 (s, 1H), 3.80 |
| | | | | (d, J = 12.8 Hz, 2H), 2.82-2.76 (m, 2H), 2.35 (s, 3H), 2.23- |
| | | | | 2.14 (m, 4H), 2.10-2.03 (m, 5H), 1.93-1.88 (m, 4H), 1.74- |
| | | | | 1.62 (m, 8H), 1.58-1.43 (m, 7H), 1.23 (s, 2H), 1.04-1.01 |
| | | | | (m, 2H) |
| I-285 | PY | MZ | 914.2 | 11.09 (s, 1H), 9.82-9.66 (m, 1H), 8.84-8.72 (m, 1H), 8.22- |
| | | | | 8.17 (m, 1H), 7.81-7.62 (m, 3H), 6.84 (d, J = 8.4 Hz, 1H), |
| | | | | 6.77-6.64 (m, 1H), 5.39-5.26 (m, 1H), 4.18-4.00 (m, |
| | | | | 1H), 3.46-3.45 (m, 3H), 2.94-2.83 (m, 1H), 2.68-2.66 |
| | | | | (m, 1H), 2.64-2.57 (m, 4H), 2.35-2.31 (m, 4H), 2.29- |
| | | | | 2.16 (m, 4H), 2.13-1.99 (m, 6H), 1.95-1.89 (m, 2H), 1.77- |
| | | | | 1.74 (m, 2H), 1.59-1.50 (m, 6H), 1.40-1.38 (m, 1H), |
| | | | | 1.33-1.17 (m, 3H), 0.68-0.53 (m, 1H), 0.37-0.17 (m, |
| | | | | 2H), 0.14--0.03 (m, 2H) |
| I-286 | NI | MZ | 879.4 | 11.09 (s, 1H), 8.74 (s, 1H), 8.53-8.50 (m, 1H), 8.13-8.07 |
| | | | | (m, 1H), 7.83-7.79 (m, 1H), 7.68 (d, J = 2.0 Hz, 1H), 7.61 |
| | | | | (dd, J = 2.0, 8.4 Hz, 1H), 7.03 (s, 1H), 6.87-6.82 (m, 1H), |
| | | | | 6.72 (t, J = 8.0 Hz, 1H), 6.37-6.34 (m, 1H), 5.36-5.29 (m, |
| | | | | 1H), 5.28-5.21 (m, 1H), 4.06 (t, J = 8.8 Hz, 1H), 3.48- |
| | | | | 3.45 (m, 3H), 3.22 (d, J = 11.2 Hz, 2H), 2.95-2.83 (m, 2H), |
| | | | | 2.69-2.60 (m, 5H), 2.38 (s, 3H), 2.20-2.06 (m, 6H), 2.03- |
| | | | | 1.85 (m, 9H), 1.81-1.75 (m, 3H), 1.71-1.59 (m, 6H), |
| | | | | 1.37-1.26 (m, 2H) |
| I-505 | QD | MZ | 940.4 | 11.09 (s, 1H), 8.98 (s, 1H), 8.34 (s, 1H), 8.06 (d, J = 8.4 Hz, |
| | | | | 1H), 7.66 (s, 1H), 7.61-7.59 (m, 1H), 6.84 (d, J = 8.8 Hz, |
| | | | | 1H), 6.76-6.68 (m, 1H), 5.34-5.29 (m, 1H), 4.42 (s, 1H), |
| | | | | 4.07-4.03 (m, 1H), 3.68-3.65 (m, 1H), 3.60-3.53 (m, |
| | | | | 2H), 3.46-3.45 (m, 6H), 3.21 (d, J = 11.2 Hz, 2H), 2.88 (s, |
| | | | | 1H), 2.70-2.57 (m, 7H), 2.36 (s, 3H), 2.24-2.14 (m, 2H), |
| | | | | 2.10-2.03 (m, 2H), 2.01-1.88 (m, 4H), 1.83-1.73 (m, |
| | | | | 3H), 1.68-1.53 (m, 5H), 1.28-1.26 (m, 2H), 1.12 (d, J = |
| | | | | 4.0 Hz, 6H) |
| I-506 | QN | MZ | 940.4 | 11.10 (s, 1H), 9.24 (s, 1H), 8.40 (s, 1H), 7.90 (d, J = 8.4 Hz, |
| | | | | 1H), 7.69 (s, 1H), 7.63 (d, J = 9.6 Hz, 1H), 6.84 (d, J = 8.4 |
| | | | | Hz, 1H), 6.72 (t, J = 8.0 Hz, 1H), 5.33 (dd, J = 5.2, 12.8 Hz, |
| | | | | 1H), 4.83 (s, 1H), 4.07 (t, J = 8.4 Hz, 1H), 3.97 (d, J = 11.6 |
| | | | | Hz, 1H), 3.76 (d, J = 12.8 Hz, 1H), 3.45-3.41 (s, 3H), 3.21 |
| | | | | (d, J = 11.2 Hz, 2H), 2.91-2.76 (m, 2H), 2.35 (s, 3H), 2.24- |
| | | | | 1.88 (m, 13H), 1.81-1.64 (m, 5H), 1.64-1.44 (m, 6H), |
| | | | | 1.32-1.12 (m, 4H), 0.95 (s, 3H), 0.81 (d, J = 6.4 Hz, 3H) |
| I-518 | NC | MZ | 926.4 | 11.09 (s, 1H), 9.12 (s, 1H), 8.36 (s, 1H), 7.96 (d, J = 8.4 Hz, |
| | | | | 1H), 7.67 (s, 1H), 7.65-7.59 (m, 1H), 6.83 (d, J = 8.4 Hz, |
| | | | | 1H), 6.71 (t, J = 7.6 Hz, 1H), 5.36-5.28 (m, 1H), 4.45 (s, |
| | | | | 1H), 4.12-4.01 (m, 1H), 3.66-3.59 (m, 1H), 3.50-3.40 |
| | | | | (m, 6H), 3.29 (s, 2H), 3.25 (s, 1H), 3.20 (d, J = 10.4 Hz, |
| | | | | 2H), 2.94-2.84 (m, 1H), 2.73-2.66 (m, 1H), 2.66-2.56 |
| | | | | (m, 4H), 2.35 (s, 3H), 2.24-2.19 (m, 2H), 2.11 (d, J = 6.4 |
| | | | | Hz, 2H), 2.08-2.00 (m, 3H), 1.94-1.87 (m, 2H), 1.84- |
| | | | | 1.78 (m, 1H), 1.75 (d, J = 12.8 Hz, 2H), 1.59-1.54 (m, 4H), |
| | | | | 1.49 (s, 2H), 1.29-1.19 (m, 2H), 1.04 (s, 3H) |
| I-555^{c} | MT | MV | 881.4 | (CD₃OD) δ = 8.43 (s, 1H), 7.99 (d, J = 8.4 Hz, 1H), 7.80- |
| | | | | 7.73 (m, 2H), 7.08-6.97 (m, 2H), 6.88 (d, J = 7.4 Hz, 1H), |
| | | | | 5.64-5.61 (m, 1H), 5.09 (dd, J = 10.9, 5.0 Hz, 2H), 4.06- |
| | | | | 3.86 (m, 6H), 3.76 (s, 3H), 3.18-3.14 (m, 2H), 2.99-2.88 |
| | | | | (m, 1H), 2.87-2.74 (m, 4H), 2.55-2.45 (m, 2H), 2.43 (s, |
| | | | | 3H), 2.38-2.30 (m, 2H), 2.29-2.21 (m, 3H), 2.20-2.11 |
| | | | | (m, 2H), 2.10-2.03 (m, 2H), 1.93-1.84 (m, 2H), 1.79- |
| | | | | 1.84 (m, 6H), 1.52-1.40 (m, 2H). |
| I-609 | NQ | MZ | 924.8 | 11.09 (s, 1H), 8.92 (s, 1H), 8.29 (s, 1H), 7.99 (d, J = 8.8 Hz, |
| | | | | 1H), 7.94 (s, 1H), 7.66 (d, J = 1.6 Hz, 1H), 7.60 (dd, J = 2.0, |
| | | | | 8.6 Hz, 1H), 6.83 (d, J = 8.6 Hz, 1H), 6.76-6.66 (m, 1H), |
| | | | | 5.32 (dd, J = 5.6, 13.0 Hz, 1H), 4.74-4.51 (m, 2H), 4.10- |
| | | | | 4.01 (m, 2H), 3.46 (d, J = 1.6 Hz, 3H), 3.19 (s, 2H), 2.92- |
| | | | | 2.84 (m, 1H), 2.63 (s, 2H), 2.60 (s, 2H), 2.42 (s, 1H), 2.36 |
| | | | | (s, 3H), 2.19 (d, J = 8.4 Hz, 3H), 2.15 (s, 1H), 2.10 (d, J = |
| | | | | 6.8 Hz, 2H), 2.04 (d, J = 8.8 Hz, 2H), 2.01-1.97 (m, 1H), |
| | | | | 1.93-1.87 (m, 2H), 1.78-1.72 (m, 3H), 1.68 (d, J = 3.6 Hz, |
| | | | | 2H), 1.63 (s, 2H), 1.60-1.46 (m, 9H), 1.35-1.12 (m, 4H) |
| I-763 | NO | MZ | 926.4 | 11.09 (s, 1H), 9.13 (s, 1H), 8.36 (s, 1H), 7.96 (d, J = 8.4 Hz, |
| | | | | 1H), 7.69-7.58 (m, 2H), 6.82 (s, 1H), 6.71 (s, 1H), 5.34- |
| | | | | 5.30 (m, 1H), 4.46 (s, 1H), 4.11-4.02 (m, 1H), 3.61-3.60 |
| | | | | (m, 2H), 3.46 (s, 3H), 3.21-3.19 (m, 4H), 2.93-2.83 (m, |
| | | | | 1H), 2.69-2.66 (m, 1H), 2.64-2.57 (m, 4H), 2.37-2.34 |
| | | | | (m, 2H), 2.33-2.31 (m, 1H), 2.26-2.18 (m, 3H), 2.12 (s, |
| | | | | 2H), 2.07 (s, 1H), 2.04-2.02(m, 3H), 1.94-1.88 (m, 2H), |
| | | | | 1.79-1.71 (m, 3H), 1.60-1.52 (m, 5H), 1.50 (s, 2H), 1.26- |
| | | | | 1.23(m, 2H), 1.04 (s, 3H) |
| I-764 | NN | MZ | 926.5 | 1.04 (s, 3H), 1.19-1.29 (m, 2H), 1.49 (d, J = 4.8 Hz, 2H), |
| | | | | 1.53-1.59 (m, 4H), 1.70-1.79 (m, 3H), 1.88-1.94 (m, |
| | | | | 2H), 1.96-2.08 (m, 4H), 2.11 (d, J = 6.4 Hz, 2H), 2.17- |
| | | | | 2.27 (m, 3H), 2.33 (m, 1H), 2.35 (s, 3H), 2.56-2.65 (m, |
| | | | | 4H), 2.65-2.70 (m, 1H), 2.83-2.92 (m, 1H), 3.20 (d, J = |
| | | | | 10.8 Hz, 4H), 3.46 (d, J = 1.6 Hz, 3H), 3.58-3.67 (m, 2H), |
| | | | | 4.01-4.12 (m, H), 4.41-4.54 (m, 1 H), 5.29-5.36 (m, 1H), |
| | | | | 6.71 (t, J = 8.4 Hz, 1H), 6.83 (d, J = 8.4 Hz, 1H) 7.58-7.70 |
| | | | | (m, 2H), 7.96 (d, J = 8.4 Hz, 1H), 8.36 (s, 1H) 9.13 (s, 1H), |
| | | | | 11.09 (s, 1H) |
| I-783 | QG | MZ | 940.3 | 9.00 (s, 1H), 8.34 (s, 1H), 8.06 (d, J = 8.8 Hz, 1H), 7.66 (s, |
| | | | | 1H), 7.61-7.59 (m, 1H), 6.84 (d, J = 9.2 Hz, 1H), 6.76- |
| | | | | 6.68 (m, 1H), 5.33-5.31 (m, 1H), 4.44 (s, 1H), 4.10-3.98 |
| | | | | (m, 1H), 3.70-3.65 (m, 1H), 3.61-3.54 (m, 2H), 3.46- |
| | | | | 3.45 (m, 6H), 3.22-3.18 (m, 2H), 2.90-2.85 (m, 1H), 2.74- |
| | | | | 2.58 (m, 7H), 2.36 (s, 3H), 2.24-2.16 (m, 2H), 2.12-2.06 |
| | | | | (m, 2H), 2.02-1.90 (m, 4H), 1.82-1.74 (m, 3H), 1.70- |
| | | | | 1.53 (m, 5H), 1.33-1.23 (m, 2H), 1.12-1.11 (m, 6H) |
| I-784 | NO | MV | 908.3 | 11.09 (s, 1H), 9.14 (s, 1H), 8.36 (s, 1H), 8.02-7.93 (m, 1H), |
| | | | | 7.68-7.67 (m, 1H), 7.64-7.61 (m, 1H), 7.00-6.94 (m, |
| | | | | 1H), 6.90-6.84 (m, 2H), 5.34-5.32 (m, 1H), 4.47 (s, 1H), |
| | | | | 4.12-4.04 (m, 1H), 3.61 (s, 3H), 3.43-3.40 (m, 2H), 3.12- |
| | | | | 3.10 (m, 2H), 2.91-2.83 (m, 2H), 2.71-2.62 (m, 8H), 2.35 |
| | | | | (s, 3H), 2.13-2.04 (m, 3H), 2.02-1.92 (m, 4H), 1.83-1.74 |
| | | | | (m, 4H), 1.66-1.55 (m, 6H), 1.51-1.45 (m, 1H), 1.40- |
| | | | | 1.21 (m, 3H), 1.06-1.02 (m, 3H) |
| I-801 | RP | RO | 861.2 | 10.83 (s, 1H), 9.11 (s, 1H), 8.35 (s, 1H), 7.85 (d, J = 8.4 Hz, |
| | | | | 1H), 7.72-7.58 (m, 2H), 7.56-7.23 (m, 1H), 7.10 (d, J = |
| | | | | 8.4 Hz, 1H), 6.97-6.84 (m, 2H), 4.45 (s, 1H), 4.04 (dd, J = |
| | | | | 5.2, 12.0 Hz, 1H), 3.72 (d, J = 11.6 Hz, 2H), 3.65-3.54 (m, |
| | | | | 1H), 3.40 (d, J = 13.2 Hz, 1H), 3.30-3.18 (m, 3H), 3.15- |
| | | | | 2.92 (m, 2H), 2.80-2.62 (m, 4H), 2.49-2.43 (m, 1H), 2.33 |
| | | | | (s, 3H), 2.30-2.13 (m, 2H), 2.12-1.84 (m, 4H), 1.84-1.69 |
| | | | | (m, 4H), 1.69-1.48 (m, 4H), 1.47-1.38 (m, 1H), 1.21 (s, |
| | | | | 2H), 1.10-0.81 (m, 6H) |
| I-818 | PH | SU | 888.0 | 10.82 (s, 1H), 10.09 (s, 1H), 8.79 (s, 1H), 8.29 (s, 1H), 8.09- |
| | | | | 8.04 (m, 1H), 7.96 (s, 1H), 7.73 (d, J = 7.2 Hz, 1H), 7.70 |
| | | | | (s, 1H), 7.67 (d, J = 2.8 Hz, 1H), 7.10 (d, J = 8.8 Hz, 1H), |
| | | | | 6.91 (d, J = 2.0 Hz, 1H), 6.86 (dd, J = 2.4, 8.8 Hz, 1H), 4.63- |
| | | | | 4.51 (m, 1H), 4.03 (dd, J = 4.8, 12.4 Hz, 1H), 4.00-3.94 |
| | | | | (m, 2H), 3.52-3.42 (m, 2H), 3.10 (s, 4H), 3.01-2.88 (m, |
| | | | | 1H), 2.79-2.64 (m, 1H), 2.39 (s, 4H), 2.26-2.16 (m, 1H), |
| | | | | 2.05-2.03 (m, 2H), 2.01-1.90 (m, 5H), 1.71-1.65 (m, |
| | | | | 4H), 1.44-1.33 (m, 1H), 1.25-1.08 (m, 3H), 0.89-0.76 |
| | | | | (m, 2H) |
| I-819 | NN | MV | 908.5 | 11.08 (s, 1H), 9.13 (s, 1H), 8.36 (s, 1H), 7.96 (d, J = 8.4 Hz, |
| | | | | 1H), 7.68 (d, J = 1.6 Hz, 1H), 7.64-7.60 (m, 1H), 6.99- |
| | | | | 6.93 (m, 1H), 6.89-6.84 (m, 2H), 5.38-5.30 (m, 1H), 4.46 |
| | | | | (s, 1H), 4.12-4.02 (m, 1H), 3.61 (s, 3H), 3.44-3.40 (m, |
| | | | | 2H), 3.26 (s, 1H), 3.09 (d, J = 9.6 Hz, 2H), 2.92-2.84 (m, |
| | | | | 1H), 2.73 (s, 5H), 2.59 (s, 1H), 2.35 (s, 3H), 2.33-2.25 (m, |
| | | | | 3H), 2.23-2.16 (m, 2H), 2.09-2.03 (m, 2H), 2.02-1.88 |
| | | | | (m, 4H), 1.81-1.75 (m, 3H), 1.61-1.50 (m, 7H), 1.29 (d, J = |
| | | | | 10.4 Hz, 2H), 1.04 (s, 3H) |
| I-1092 | TF | PM | 853.2 | 10.80 (s, 1H), 9.57 (s, 1H), 8.39 (s, 1H), 8.13-8.10 (m, 1H), |
| | | | | 8.01-8.00 (m, 1H), 7.72-7.60 (m, 2H), 7.04-6.89 (m, |
| | | | | 3H), 4.62-4.37 (m, 2H), 3.80-3.76 (m, 1H), 3.67-3.63 |
| | | | | (m, 1H), 3.51-3.37 (m, 2H), 3.29 (s, 4H), 3.03-2.92 (m, |
| | | | | 1H), 2.71-2.54 (m, 4H), 2.46-2.45 (m, 2H), 2.19-2.12 |
| | | | | (m, 3H), 2.00-1.95 (m, 2H), 1.83-1.64 (m, 4H), 1.62- |
| | | | | 1.51 (m, 3H), 1.48-1.45 (m, 1H), 1.35-1.14 (m, 3H), 1.04 |
| | | | | (s, 3H) |
| I-1051 | TK | TG | 853.3 | 10.86-10.73 (m, 1H), 9.71-9.46 (m, 1H), 8.42 (s, 1H), |
| | | | | 8.16-7.95 (m, 2H), 7.73-7.59 (m, 2H), 7.05 (t, J = 8.8 Hz, |
| | | | | 1H), 6.69 (d, J = 9.6 Hz, 2H), 4.98-4.37 (m, 2H), 3.90- |
| | | | | 3.84 (m, 1H), 3.81-3.59 (m, 5H), 3.43-3.35 (m, 3H), 3.27- |
| | | | | 3.16 (m, 1H), 3.03-2.94 (m, 1H), 2.77-2.61 (m, 5H), |
| | | | | 2.20-2.08 (m, 3H), 2.00-1.91 (m, 2H), 1.79-1.59 (m, |
| | | | | 4H), 1.51 (s, 4H), 1.26 (dd, J = 1.6, 9.2 Hz, 1H), 1.15 (s, |
| | | | | 3H), 1.11 (s, 1H) |
| I-1050 | TK | PM | 853.2 | 10.80 (s, 1H), 9.61 (s, 1H), 8.41 (s, 1H), 8.04-7.99 (m, 2H), |
| | | | | 7.70-7.62 (m, 2H), 7.01-6.91 (m, 3H), 4.58-4.41 (m, |
| | | | | 2H), 3.81-3.76 (m, 1H), 3.74-3.71 (m, 2H), 3.43-3.36 |
| | | | | (m, 2H), 3.30-3.20 (m, 4H), 3.04-2.91 (m, 1H), 2.72- |
| | | | | 2.66 (m, 1H), 2.64-2.56 (m, 3H), 2.23-2.11 (m, 4H), 2.02- |
| | | | | 1.91 (m, 2H), 1.72-1.72 (m, 2H), 1.59-1.54 (m, 1H), |
| | | | | 1.50-1.50 (m, 4H), 1.31-1.15 (m, 4H), 1.14 (s, 3H) |
| I-1046 | TH | TG | 849.3 | 10.79 (s, 1H), 9.58 (s, 1H), 8.39 (s, 1H), 8.15-8.08 (m, 1H), |
| | | | | 7.79-7.68 (m, 1H), 7.66-7.58 (m, 2H), 7.05 (t, J = 8.7 Hz, |
| | | | | 1H), 6.69 (d, J = 10.0 Hz, 2H), 4.47 (s, 1H), 3.86 (dd, J = |
| | | | | 5.2, 12.4 Hz, 1H), 3.73-3.62 (m, 3H), 3.45-3.40 (m, 2H), |
| | | | | 3.28 (s, 4H), 2.77-2.61 (m, 5H), 2.49-2.47 (m, 1H), 2.18- |
| | | | | 2.07 (m, 2H), 1.98-1.87 (m, 2H), 1.81-1.68 (m, 4H), |
| | | | | 1.65-1.38 (m, 6H), 1.23-1.10 (m, 2H), 1.04 (s, 3H), 0.76 |
| | | | | (d, J = 2.0 Hz, 3H) |
| I-1027 | TF | TM | 835.2 | 10.76 (s, 1H), 9.58 (s, 1H), 8.39 (s, 1H), 8.14-8.05 (m, 1H), |
| | | | | 8.01 (dd, J = 2.0, 3.6 Hz, 1H), 7.70-7.59 (m, 2H), 7.02 (d, |
| | | | | J = 8.4 Hz, 2H), 6.86 (d, J = 8.8 Hz, 2H), 4.60-4.38 (m, |
| | | | | 2H), 3.70 (dd, J = 4.8, 10.8 Hz, 1H), 3.63 (d, J = 11.2 Hz, |
| | | | | 3H), 3.45-3.39 (m, 1H), 3.29-3.25 (m, 2H), 3.02-2.91 |
| | | | | (m, 1H), 2.71-2.54 (m, 4H), 2.47-2.40 (m, 2H), 2.20- |
| | | | | 2.05 (m, 4H), 2.03-1.89 (m, 2H), 1.83-1.65 (m, 4H), 1.61- |
| | | | | 1.52 (m, 3H), 1.46 (dd, J = 3.2, 6.0 Hz, 1H), 1.35-1.22 |
| | | | | (m, 1H), 1.21-1.08 (m, 2H), 1.04 (s, 3H) |
| I-1026 | TF | TL | 871.4 | 10.85 (s, 1H), 9.56 (s, 1H), 8.39 (s, 1H), 8.10 (t, J = 8.0 Hz, |
| | | | | 1H), 8.00 (d, J = 7.6 Hz, 1H), 7.70-7.60 (m, 2H), 6.58 (d, |
| | | | | J = 12.8 Hz, 2H), 4.54 (s, 1H), 4.44 (d, J = 15.2 Hz, 1H), |
| | | | | 4.07-3.98 (m, 1H), 3.78-3.59 (m, 4H), 3.46-3.38 (m, |
| | | | | 2H), 3.24 (s, 2H), 3.02-2.90 (m, 1H), 2.83-2.61 (m, 5H), |
| | | | | 2.14-2.02 (m, 4H), 1.97-1.90 (m, 2H), 1.83-1.74 (m, |
| | | | | 1H), 1.71-1.60 (m, 4H), 1.59-1.54 (m, 2H), 1.49-1.42 |
| | | | | (m, 1H), 1.30-1.23 (m, 1H), 1.08 (s, 1H), 1.04 (s, 3H) |
| I-1017 | TN | TF | 889.4 | 10.84 (s, 1H), 9.57 (s, 1H), 8.42-8.37 (m, 1H), 8.10 (t, |
| | | | | J = 8.0 Hz, 1H), 8.01 (d, J = 8.0 Hz, 1H), 7.69-7.60 |
| | | | | (m, 2H), 7.46 (d, J = 9.2 Hz, 1H), 6.88 (d, J = 9.2 Hz, |
| | | | | 1H), 6.80 (s, 1H), 4.58-4.41 (m, 2H), 4.24 (dd, J = |
| | | | | 5.2, 9.2 Hz, 1H), 3.87 (s, 3H), 3.75 (d, J = 11.6 Hz, |
| | | | | 2H), 3.69-3.63 (m, 1H), 3.41 (s, 1H), 3.28 (s, 2H), |
| | | | | 3.03-2.93 (m, 1H), 2.74-2.65 (m, 3H), 2.64-2.57 |
| | | | | (m, 2H), 2.33-2.25 (m, 1H), 2.22-2.06 (m, 4H), 1.94 |
| | | | | (t, J = 11.2 Hz, 1H), 1.86-1.52 (m, 8H), 1.51-1.42 (m, |
| | | | | 1H), 1.32-1.25 (m, 1H), 1.19 (q, J = 11.2 Hz, 2H), |
| | | | | 1.04 (s, 3H) |

^{a}The reductive amination was performed under standard conditions from −10 C. to 25° C. for 1-2 hrs. TEA and HOAc were also used as the base in place of KOAc. The final products were purified under standard techniques including prep-HPLC and chromatography.
^{b}The product of the reductive amination was further deprotected with TfOH/TFA at 70° C. for 0.5-1 hr. The final compound was then purified via prep-HPLC.
^{c}The 1H NMR data was reported with CD₃OD as the solvent.

### Example 5. Preparation of Compounds of the Invention (Alternative Methods)

### Syntheses of N-[4-[[4-[3-chloro-4-[(3S)-2,6-dioxo-3-piperidyl]phenyl]piperazin-1-yl]methyl]cyclohexyl]-4-[[4-[(3S)-3-hydroxy-3-methyl-1-piperidyl]-5-(trifluoromethyl)pyrimidin-2-yl]amino]-3-methyl-benzenesulfonamide (I-809) and N-[4-[[4-[3-chloro-4-[(3R)-2,6-dioxo-3-piperidyl]phenyl] piperazin-1-yl]methyl]cyclohexyl]-4-[[4-[(3S)-3-hydroxy-3-methyl-1-piperidyl]-5-(trifluoromethyl)pyrimidin-2-yl]amino]-3-methyl-benzenesulfonamide (I-817)

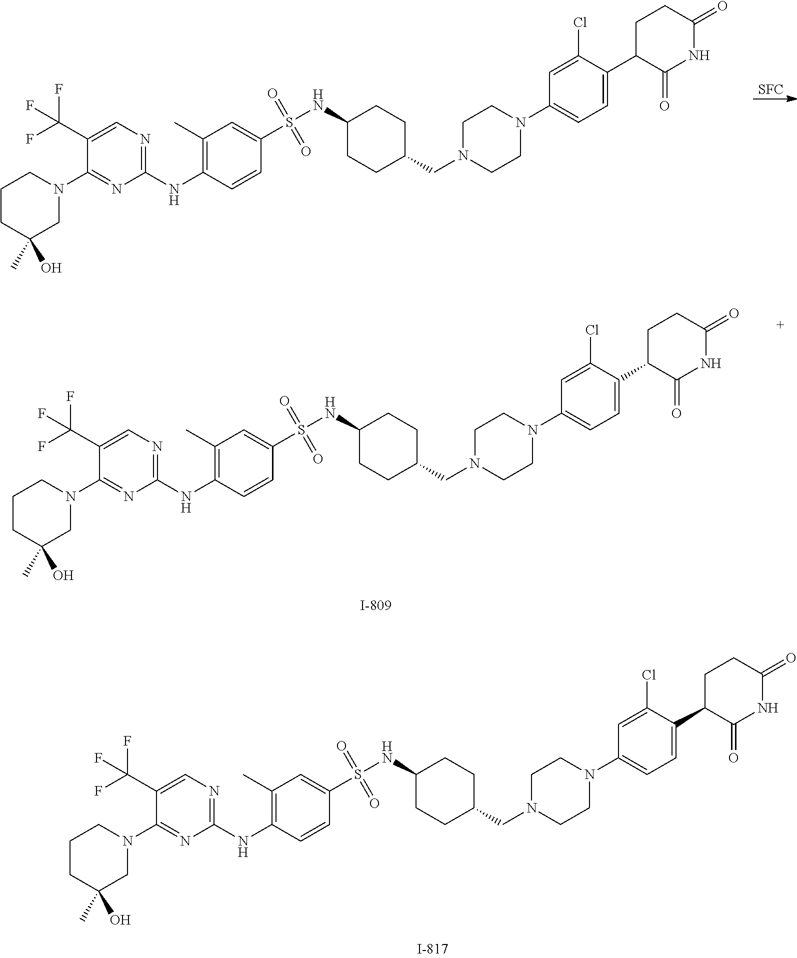 [SMILES: Cc1cc(S(=O)(=O)N[C@H]2CC[C@H](CN3CCN(c4ccc(C5CCC(=O)NC5=O)c(Cl)c4)CC3)CC2)ccc1Nc1ncc(C(F)(F)F)c(N2CCC[C@](C)(O)C2)n1 ; "SFC" ; Cc1cc(S(=O)(=O)N[C@H]2CC[C@H](CN3CCN(c4ccc([C@@H]5CCC(=O)NC5=O)c(Cl)c4)CC3)CC2)ccc1Nc1ncc(C(F)(F)F)c(N2CCC[C@](C)(O)C2)n1 ; Cc1cc(S(=O)(=O)N[C@H]2CC[C@H](CN3CCN(c4ccc([C@H]5CCC(=O)NC5=O)c(Cl)c4)CC3)CC2)ccc1Nc1ncc(C(F)(F)F)c(N2CCC[C@](C)(O)C2)n1]

N-[4-[[4-[3-chloro-4-(2,6-dioxo-3-piperidyl)phenyl]piperazin-1-yl]methyl]cyclohexyl]-4-[[4-[(3S)-3-hydroxy-3-methyl-1-piperidyl]-5-(trifluoromethyl)pyrimidin-2-yl]amino]-3-methyl-benzenesulfonamide (250 mg, 295 μmol, I-745) was separated by SFC (column: REGIS (R,R)WHELK-01 (250 mm*25 mm, 10 um); mobile phase: [CO₂-ACN/MeOH (0.1% NH₃—H₂O)]; B %:70%, isocratic elution mode) to give two peaks. The absolute stereochemistry of the diastereomers was assigned arbitrarily. N-[4-[[4-[3-chloro-4-[(3S)-2,6-dioxo-3-piperidyl]phenyl]piperazin-1-yl]methyl]cyclohexyl]-4-[[4-[(3S)-3-hydroxy-3-methyl-1-piperidyl]-5-(trifluoromethyl)pyrimidin-2-yl]amino]-3-methyl-benzenesulfonamide (99.5 mg, 40% yield, peak 1) was further purified by prep-HPLC (column: Waters xbridge 150*25 mm 10 um; mobile phase: [water(NH₄HCO₃)-ACN]; gradient:46%-76% B over 10 min) to give N-[4-[[4-[3-chloro-4-[(3S)-2,6-dioxo-3-piperidyl]phenyl]piperazin-1-yl]methyl]cyclohexyl]-4-[[4-[(3S)-3-hydroxy-3-methyl-1-piperidyl]-5-(trifluoromethyl)pyrimidin-2-yl]amino]-3-methyl-benzenesulfonamide (99.5 mg, 40% yield) as white solid. ¹H NMR (400 MHz, DMSO-d₆) δ 10.82 (s, 1H), 9.09 (s, 1H), 8.34 (s, 1H), 7.82 (d, J=8.4 Hz, 1H), 7.64 (d, J=2.0 Hz, 1H), 7.60-7.57 (m, 1H), 7.52 (s, 1H), 7.10 (d, J=8.8 Hz, 1H), 6.91 (d, J=2.4 Hz, 1H), 6.87-6.84 (m, 1H), 4.45 (s, 1H), 4.05-4.01 (m, 1H), 3.65-3.56 (m, 1H), 3.41-3.38 (m, 1H), 3.28-3.20 (m, 2H), 3.11 (s, 4H), 2.91-2.82 (m, 1H), 2.78-2.66 (m, 1H), 2.47-2.46 (m, 1H), 2.39 (s, 4H), 2.32 (s, 3H), 2.23-2.19 (m, 1H), 2.04-2.03 (m, 2H), 1.98-1.86 (m, 1H), 1.80-1.73 (m, 1H), 1.72-1.62 (m, 4H), 1.59-1.51 (m, 2H), 1.45-1.31 (m, 2H), 1.19-1.08 (m, 2H), 1.03 (s, 3H), 0.85-0.71 (m, 2H); LC-MS (ESI⁺) m/z 847.1 (M+H)⁺.

N-[4-[[4-[3-chloro-4-[(3R)-2,6-dioxo-3-piperidyl]phenyl]piperazin-1-yl]methyl] cyclohexyl]-4-[[4-[(3S)-3-hydroxy-3-methyl-1-piperidyl]-5-(trifluoromethyl) pyrimidin-2-yl]amino]-3-methyl-benzenesulfonamide (100 mg, 118 μmol, peak 2) was further purified by prep-HPLC (column: Waters xbridge 150*25 mm 10 um; mobile phase: [water(NH₄HCO₃)-ACN]; gradient:46%-76% B over 10 min) to give N-[4-[[4-[3-chloro-4-[(3R)-2,6-dioxo-3-piperidyl]phenyl]piperazin-1-yl]methyl]cyclohexyl]-4-[[4-[(3S)-3-hydroxy-3-methyl-1-piperidyl]-5-(trifluoromethyl)pyrimidin-2-yl]amino]-3-methyl-benzenesulfonamide (78.1 mg, 78% yield) as white solid. ¹H NMR (400 MHz, DMSO-d₆) δ 10.82 (s, 1H), 9.09 (s, 1H), 8.34 (s, 1H), 7.83 (d, J=8.4 Hz, 1H), 7.64 (d, J=2.0 Hz, 1H), 7.60-7.57 (m, 1H), 7.52 (d, J=6.0 Hz, 1H), 7.10 (d, J=8.8 Hz, 1H), 6.91 (d, J=2.4 Hz, 1H), 6.87-6.84 (m, 1H), 4.45 (s, 1H), 4.05-4.01 (m, 1H), 3.61-3.58 (m, 1H), 3.41-3.38 (m, 1H), 3.28-3.20 (m, 2H), 3.11 (s, 4H), 2.92-2.82 (m, 1H), 2.78-2.67 (m, 1H), 2.47-2.46 (m, 1H), 2.39 (s, 4H), 2.32 (s, 3H), 2.23-2.19 (m, 1H), 2.04-2.03 (m, 2H), 1.97-1.88 (m, 1H), 1.80-1.73 (m, 1H), 1.69-1.62 (m, 4H), 1.59-1.50 (m, 2H), 1.46-1.30 (m, 2H), 1.20-1.07 (m, 2H), 1.03 (s, 3H), 0.85-0.70 (m, 2H); LC-MS (ESI⁺) m/z 847.1 (M+H)⁺.

### Example 6. CDK2 degradation in MKN1 cells

Degradation of CDK2 in MKN1 cells was measured using AlphaLISA technology (PerkinElmer ALSU-TCDK2-A10K). MKN1 cells were maintained in RPMI1640 medium containing 10% FBS+1× penn/strep. Cells were seeded in 96-well plates (Corning 3599) at a density of 2e4 cells per well in 80 μL of fresh complete growth medium. Cells were incubated overnight at 37° C., 5% CO₂. Compounds were then added to the cell assay plates with a final top concentration of 10 μM in a 1:5 dilution series with a total of 11 doses (0.001 nM-10 μM). The final volume per well was 160 μL and the final DMSO concentration was 0.1%. Additionally, wells containing no cells and only complete growth medium were also maintained on the cell assay plates (‘no cell’ wells) and processed along with experimental wells. After 14 hour-incubation at 37° C., 5% CO₂, the medium was removed and the cells were washed once with 100 μL of 1×PBS. Following removal of PBS, 40 μL of 1× Lysis Buffer containing 1× protease and phosphatase inhibitors (Roche 4693116001; Roche 4906837001) were added to each well. The cell assay plate was agitated on a plate shaker at 350 rpm for 10 minutes at room temperature. 10 μL of each cell lysate were transferred to a 384-well plate (PerkinElmer 6007290). 10 μL of control lysates (from PerkinElmer ALSU-TCDK2-A10K kit) were added to separate wells to generate a standard curve. Activation Buffer was prepared by diluting 25-fold in combined Reaction Buffer 1 and Reaction Buffer 2. Acceptor Beads were diluted 50-fold in combined Reaction Buffers. 5 μL of Acceptor Mix were added to each well. The plate was sealed with Topseal-Aadhesive film, covered with foil, and incubated in the dark for 1 hour at room temperature. Under subdued light, the Donor Beads were diluted 50-fold in Dilution Buffer. 5 μL of Donor Mix were added to each well under subdued light. The plate was sealed with Topseal-A adhesive film, covered with foil, and incubated in the dark for 1 hour at room temperature. Under subdued light, the plates were analyzed on an EnVision Microplate Reader (PerkinElmer Model 2009-0030). The average signal from ‘no cell’ wells was subtracted from the signal in experimental wells prior to calculating Alpha signal for test compounds and negative control (0.100 DMSO). The data were analyzed using Xlfit (v5.3. 1.3) and the dose-dependent CDK2 degradation data were fit using a four-parameter logistic model to calculate DC₅₀.

Table 4 shows the results of CDK2 degradation in MKN1 cells. The letter codes for CDK2 degraderation include: A (<100 nM), B (≥100-500 nM), C (>500-1000 nM), D (>1000 nM), and E (not achieved). The letter codes for average Dmax % include: A (>75% o), B (>50% -75%), C (>25% -50%), D (<25%) and E (not tested).

TABLE 4
CDK2 degradation in MKN1 cells

| | CDK2 | |
|---|---|---|
| | degradation | CDK2 |
| | 14 h: Mean | degradation |
| | external-Abs | 14 h: Average |
| I-#^{a} | DC₅₀ (nM) | Dmax % |
| I-1 | A | B |
| I-2 | A | A |
| I-3 | A | A |
| I-4 | A | B |
| I-6 | E | B |
| I-7 | A | A |
| I-8 | B | B |
| I-10 | A | A |
| I-11 | A | A |
| I-12 | A | A |
| I-14 | D | B |
| I-33 | A | A |
| I-34 | B | A |
| I-35 | A | B |
| I-36 | A | B |
| I-37 | B | B |
| I-38 | A | A |
| I-39 | A | B |
| I-40 | A | A |
| I-41 | A | A |
| I-42 | A | A |
| I-43 | B | A |
| I-44 | A | A |
| I-45 | B | A |
| I-46 | A | A |
| I-47 | A | A |
| I-48 | A | B |
| I-49 | A | B |
| I-50 | A | A |
| I-51 | A | A |
| I-52 | B | B |
| I-53 | A | A |
| I-54 | A | A |
| I-55 | A | E |
| I-56 | A | A |
| I-57 | A | A |
| I-58 | A | A |
| I-59 | A | A |
| I-60 | A | A |
| I-61 | A | A |
| I-62 | E | C |
| I-63 | A | A |
| I-64 | A | A |
| I-65 | A | A |
| I-66 | B | A |
| I-67 | B | A |
| I-68 | A | E |
| I-69 | B | A |
| I-70 | D | B |
| I-71 | B | A |
| I-72 | B | A |
| I-73 | A | A |
| I-74 | B | A |
| I-75 | B | B |
| I-76 | C | B |
| I-77 | A | A |
| I-78 | D | D |
| I-79 | B | B |
| I-80 | E | C |
| I-81 | A | A |
| I-82 | A | A |
| I-86 | A | A |
| I-87 | A | A |
| I-88 | A | A |
| I-90 | B | B |
| I-93 | A | A |
| I-119 | A | A |
| I-125 | A | A |
| I-128 | A | A |
| I-129 | E | C |
| I-130 | D | D |
| I-152 | A | B |
| I-153 | B | B |
| I-157 | A | B |
| I-158 | A | B |
| I-162 | A | A |
| I-163 | D | B |
| I-169 | E | C |
| I-173 | D | D |
| I-174 | D | C |
| I-175 | A | A |
| I-177 | C | B |
| I-178 | B | A |
| I-180 | B | B |
| I-182 | A | A |
| I-183 | A | A |
| I-184 | A | A |
| I-191 | A | A |
| I-203 | A | A |
| I-206 | B | B |
| I-207 | A | A |
| I-259 | D | D |
| I-265 | A | A |
| I-266 | A | B |
| I-267 | A | A |
| I-272 | A | A |
| I-276 | A | A |
| I-277 | A | A |
| I-278 | A | A |
| I-280 | D | C |
| I-285 | A | A |
| I-286 | A | A |
| I-287 | A | A |
| I-295 | D | C |
| I-302 | A | A |
| I-305 | B | A |
| I-308 | A | A |
| I-309 | B | A |
| I-318 | E | C |
| I-324 | A | A |
| I-325 | A | A |
| I-329 | A | A |
| I-332 | B | A |
| I-335 | A | A |
| I-341 | A | A |
| I-342 | A | A |
| I-343 | D | B |
| I-347 | A | A |
| I-350 | D | C |
| I-351 | A | A |
| I-352 | A | A |
| I-353 | D | D |
| I-354 | D | D |
| I-355 | D | D |
| I-356 | D | D |
| I-357 | E | B |
| I-358 | C | B |
| I-359 | A | A |
| I-360 | D | D |
| I-364 | D | B |
| I-369 | A | A |
| I-375 | A | A |
| I-376 | A | A |
| I-379 | A | A |
| I-380 | D | D |
| I-381 | A | A |
| I-382 | E | C |
| I-383 | A | A |
| I-386 | D | C |
| I-387 | B | A |
| I-388 | D | D |
| I-389 | D | C |
| I-390 | A | A |
| I-391 | E | C |
| I-392 | D | D |
| I-393 | B | B |
| I-394 | A | A |
| I-395 | A | A |
| I-397 | A | A |
| I-398 | A | A |
| I-399 | A | A |
| I-400 | A | A |
| I-401 | A | B |
| I-402 | B | A |
| I-403 | B | B |
| I-404 | B | A |
| I-405 | D | B |
| I-406 | E | B |
| I-407 | B | B |
| I-408 | B | B |
| I-409 | D | C |
| I-410 | D | C |
| I-411 | D | C |
| I-412 | D | C |
| I-413 | D | B |
| I-414 | B | B |
| I-415 | A | A |
| I-416 | A | A |
| I-417 | A | A |
| I-418 | D | C |
| I-419 | A | A |
| I-420 | A | A |
| I-421 | B | B |
| I-422 | E | C |
| I-423 | A | A |
| I-424 | A | A |
| I-425 | E | C |
| I-426 | E | C |
| I-427 | D | B |
| I-428 | A | A |
| I-429 | A | A |
| I-430 | A | A |
| I-431 | A | A |
| I-432 | A | A |
| I-433 | A | A |
| I-434 | A | A |
| I-435 | A | A |
| I-436 | A | A |
| I-437 | A | A |
| I-438 | A | A |
| I-439 | A | A |
| I-440 | A | A |
| I-441 | A | A |
| I-442 | A | A |
| I-443 | A | A |
| I-444 | A | A |
| I-445 | A | A |
| I-446 | D | C |
| I-447 | D | D |
| I-448 | E | C |
| I-449 | A | A |
| I-450 | A | A |
| I-451 | E | C |
| I-452 | A | B |
| I-453 | D | D |
| I-454 | D | C |
| I-455 | D | D |
| I-456 | D | D |
| I-457 | D | D |
| I-458 | B | B |
| I-459 | B | B |
| I-460 | A | B |
| I-461 | A | A |
| I-462 | A | A |
| I-463 | E | C |
| I-464 | A | B |
| I-465 | D | B |
| I-466 | D | D |
| I-467 | E | C |
| I-468 | A | A |
| I-469 | A | A |
| I-470 | A | A |
| I-471 | A | A |
| I-472 | A | A |
| I-473 | A | A |
| I-474 | A | A |
| I-474 | A | A |
| I-474 | A | A |
| I-474 | A | A |
| I-474 | A | A |
| I-475 | A | A |
| I-477 | C | B |
| I-479 | A | B |
| I-480 | D | C |
| I-481 | A | A |
| I-483 | A | A |
| I-484 | A | A |
| I-485 | A | A |
| I-486 | A | A |
| I-487 | A | A |
| I-488 | A | A |
| I-489 | B | A |
| I-490 | A | A |
| I-491 | D | D |
| I-492 | D | D |
| I-493 | D | C |
| I-494 | D | D |
| I-495 | D | D |
| I-496 | D | D |
| I-497 | D | D |
| I-498 | D | D |
| I-499 | D | D |
| I-500 | D | D |
| I-501 | D | D |
| I-502 | D | D |
| I-503 | A | B |
| I-504 | E | C |
| I-505 | A | A |
| I-506 | A | A |
| I-507 | A | B |
| I-513 | A | B |
| I-514 | D | D |
| I-515 | A | A |
| I-516 | D | B |
| I-517 | A | A |
| I-518 | A | A |
| I-519 | A | B |
| I-524 | A | A |
| I-529 | A | A |
| I-536 | A | A |
| I-537 | A | A |
| I-539 | A | A |
| I-540 | A | A |
| I-552 | A | A |
| I-555 | A | A |
| I-593 | A | A |
| I-609 | A | A |
| I-663 | A | A |
| I-664 | A | A |
| I-665 | A | A |
| I-666 | A | A |
| I-667 | A | A |
| I-668 | A | A |
| I-669 | A | A |
| I-670 | A | A |
| I-681 | A | A |
| I-682 | A | A |
| I-683 | A | A |
| I-684 | A | A |
| I-685 | A | A |
| I-686 | A | A |
| I-687 | A | A |
| I-692 | A | A |
| I-693 | A | A |
| I-695 | D | C |
| I-697 | B | A |
| I-698 | A | A |
| I-699 | A | A |
| I-704 | A | A |
| I-712 | A | B |
| I-713 | A | A |
| I-714 | A | A |
| I-715 | A | A |
| I-716 | D | B |
| I-717 | A | A |
| I-718 | B | A |
| I-719 | A | A |
| I-720 | A | A |
| I-721 | A | A |
| I-722 | A | A |
| I-727 | A | A |
| I-728 | B | B |
| I-729 | A | A |
| I-741 | B | A |
| I-742 | C | B |
| I-745 | A | A |
| I-758 | A | A |
| I-763 | A | A |
| I-764 | D | C |
| I-765 | A | A |
| I-766 | A | A |
| I-767 | A | A |
| I-768 | A | A |
| I-769 | A | A |
| I-770 | A | A |
| I-771 | A | A |
| I-772 | A | A |
| I-773 | A | A |
| I-774 | A | A |
| I-775 | A | A |
| I-776 | A | A |
| I-777 | A | A |
| I-778 | A | A |
| I-779 | A | A |
| I-780 | A | A |
| I-781 | A | A |
| I-782 | A | A |
| I-783 | A | A |
| I-784 | A | A |
| I-785 | A | A |
| I-786 | A | A |
| I-787 | A | A |
| I-788 | A | A |
| I-789 | A | A |
| I-790 | A | A |
| I-791 | A | A |
| I-792 | A | A |
| I-793 | A | A |
| I-794 | A | A |
| I-795 | A | A |
| I-796 | A | A |
| I-797 | A | A |
| I-798 | A | A |
| I-799 | A | A |
| I-800 | A | A |
| I-801 | A | A |
| I-802 | A | A |
| I-803 | A | A |
| I-804 | A | A |
| I-805 | A | A |
| I-806 | A | A |
| I-807 | A | A |
| I-808 | A | A |
| I-809 | A | A |
| I-810 | A | A |
| I-811 | A | A |
| I-812 | A | A |
| I-813 | A | A |
| I-814 | A | A |
| I-815 | A | A |
| I-816 | A | A |
| I-817 | B | A |
| I-818 | A | A |
| I-819 | C | B |
| I-820 | A | A |
| I-821 | A | A |
| I-822 | A | A |
| I-823 | A | A |
| I-824 | E | D |
| I-825 | D | D |
| I-826 | A | B |
| I-827 | A | A |
| I-828 | A | A |
| I-829 | E | D |
| I-830 | A | A |
| I-831 | A | A |
| I-832 | A | A |
| I-833 | E | C |
| I-834 | A | A |
| I-835 | D | D |
| I-836 | B | B |
| I-837 | D | D |
| I-838 | D | D |
| I-839 | D | D |
| I-840 | A | B |
| I-841 | A | A |
| I-842 | A | A |
| I-843 | D | D |
| I-844 | A | A |
| I-845 | B | B |
| I-846 | A | A |
| I-847 | A | A |
| I-848 | A | A |
| I-849 | A | B |
| I-850 | A | B |
| I-851 | A | A |
| I-852 | A | B |
| I-853 | B | B |
| I-854 | B | B |
| I-855 | A | B |
| I-856 | D | D |
| I-857 | A | A |
| I-858 | A | A |
| I-859 | A | A |
| I-860 | A | B |
| I-861 | A | B |
| I-862 | E | C |
| I-863 | D | B |
| I-864 | A | A |
| I-865 | A | A |
| I-866 | D | D |
| I-867 | A | A |
| I-868 | A | B |
| I-869 | A | A |
| I-870 | A | A |
| I-871 | A | A |
| I-872 | A | A |
| I-873 | A | A |
| I-874 | A | A |
| I-875 | A | A |
| I-876 | A | A |
| I-877 | A | A |
| I-878 | A | A |
| I-879 | A | A |
| I-880 | A | B |
| I-881 | D | D |
| I-882 | A | A |
| I-883 | A | A |
| I-884 | D | C |
| I-885 | A | A |
| I-886 | D | C |
| I-887 | D | C |
| I-888 | D | C |
| I-889 | A | A |
| I-890 | A | A |
| I-891 | A | A |
| I-892 | A | A |
| I-893 | A | A |
| I-894 | B | B |
| I-895 | A | B |
| I-896 | B | B |
| I-897 | D | D |
| I-898 | D | D |
| I-899 | A | A |
| I-900 | A | A |
| I-901 | D | B |
| I-902 | D | D |
| I-903 | D | B |
| I-904 | D | B |
| I-905 | D | B |
| I-906 | D | B |
| I-907 | B | B |
| I-908 | A | A |
| I-909 | A | B |
| I-910 | B | B |
| I-911 | D | C |
| I-912 | D | D |
| I-913 | A | A |
| I-914 | A | B |
| I-915 | A | A |
| I-916 | A | A |
| I-917 | A | A |
| I-918 | A | A |
| I-919 | A | A |
| I-920 | A | A |
| I-921 | A | A |
| I-922 | A | B |
| I-923 | C | B |
| I-924 | D | C |
| I-925 | D | C |
| I-926 | D | C |
| I-927 | B | A |
| I-928 | A | A |
| I-929 | D | C |
| I-930 | A | A |
| I-931 | A | A |
| I-932 | A | A |
| I-933 | A | A |
| I-934 | A | A |
| I-935 | A | A |
| I-936 | A | A |
| I-937 | A | A |
| I-938 | D | C |
| I-939 | A | B |
| I-940 | A | A |
| I-941 | A | A |
| I-942 | A | A |
| I-943 | A | A |
| I-944 | A | A |
| I-945 | B | A |
| I-946 | B | B |
| I-947 | A | A |
| I-949 | A | A |
| I-950 | A | A |
| I-951 | A | A |
| I-952 | D | C |
| I-953 | A | A |
| I-954 | A | A |
| I-955 | D | C |
| I-963 | A | A |
| I-964 | A | A |
| I-965 | A | A |
| I-966 | A | A |
| I-967 | A | A |
| I-968 | A | A |
| I-969 | D | C |
| I-970 | A | A |
| I-971 | A | A |
| I-972 | A | B |
| I-973 | A | A |
| I-974 | A | A |
| I-975 | A | A |
| I-976 | A | B |
| I-977 | A | A |
| I-1006 | D | D |
| I-1007 | D | D |
| I-1008 | D | A |
| I-1009 | D | D |
| I-1010 | D | C |
| I-1011 | D | D |
| I-1012 | D | D |
| I-1013 | D | D |
| I-1014 | C | A |
| I-1036 | B | A |
| I-1037 | A | A |
| I-1038 | D | C |
| I-1039 | A | A |
| I-1040 | A | A |
| I-1041 | A | A |
| I-1042 | A | A |
| I-1043 | D | D |
| I-1044 | D | D |
| I-1045 | B | B |
| I-1046 | A | A |
| I-1047 | A | A |
| I-1048 | A | A |
| I-1049 | A | A |
| I-1050 | A | A |
| I-1051 | A | A |
| I-1054 | A | A |
| I-1055 | A | A |
| I-1056 | A | A |
| I-1057 | B | B |
| I-1058 | A | A |
| I-1059 | A | A |
| I-1060 | A | A |
| I-1061 | A | A |
| I-1062 | A | A |
| I-1063 | A | A |
| I-1064 | A | A |
| I-1065 | A | A |
| I-1066 | A | A |
| I-1067 | A | A |
| I-1068 | B | A |
| I-1069 | A | A |
| I-1070 | A | A |
| I-1071 | A | A |
| I-1072 | A | A |
| I-1073 | A | B |
| I-1074 | B | A |
| I-1075 | A | A |
| I-1076 | B | B |
| I-1077 | A | A |
| I-1078 | A | A |
| I-1079 | A | A |
| I-1080 | A | A |
| I-1081 | B | A |
| I-1089 | A | A |
| I-1090 | A | A |
| I-1091 | A | A |
| I-1092 | A | A |
| I-1093 | A | A |
| I-1094 | A | A |
| I-1095 | A | A |
| I-1096 | A | A |
| I-1097 | A | A |
| I-1098 | A | A |
| I-1099 | A | A |
| I-1100 | A | A |
| I-1101 | B | B |
| I-1102 | A | A |
| I-1103 | D | C |
| I-1104 | A | A |
| I-1105 | A | A |
| I-1106 | A | A |
| I-1107 | A | A |
| I-1108 | A | A |
| I-1109 | A | A |
| I-1110 | B | B |
| I-1111 | B | A |
| I-1112 | A | A |
| I-1113 | A | A |
| I-1114 | B | B |
| I-1115 | B | A |
| I-1116 | A | A |
| I-1117 | A | A |
| I-1118 | A | A |
| I-1119 | A | A |
| I-1120 | A | A |
| I-1121 | B | A |
| I-1122 | A | A |
| I-1124 | A | A |
| I-1125 | A | A |
| I-1126 | B | A |
| I-1127 | A | A |
| I-1128 | A | A |
| I-1129 | A | A |
| I-1130 | A | A |
| I-1131 | A | A |
| I-1132 | A | A |
| I-1133 | A | A |
| I-1134 | A | A |
| I-1135 | A | A |
| I-1136 | A | A |
| I-1137 | A | A |
| I-1138 | D | B |
| I-1139 | A | A |
| I-1140 | A | A |
| I-1141 | A | A |
| I-1142 | A | A |
| I-1143 | A | A |
| I-1144 | B | A |
| I-1145 | A | A |
| I-1146 | A | A |
| I-1147 | A | A |
| I-1148 | D | C |
| I-1149 | A | B |
| I-1150 | A | A |
| I-1152 | A | A |
| I-1153 | B | A |
| I-1154 | A | A |
| I-1155 | A | A |

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

LENGTHY TABLES
The patent application contains a lengthy table section. A copy of the table is available in electronic form from the USPTO web site (https://seqdata.uspto.gov/?pageRequest=docDetail&DocID=US20240173419A1). An electronic copy of the table will also be available from the USPTO upon request and payment of the fee set forth in 37 CFR 1.19(b)(3).

1. A compound of formula I-a′: 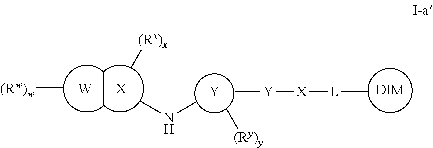
or a pharmaceutically acceptable salt thereof, wherein:
Ring W and Ring X are independently fused rings selected from benzo, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Ring Y is a ring selected from phenylenyl, a 4 to 7-membered saturated or partially unsaturated carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
Y is a covalent bond, —S(O)₂—, —S(O)—, —S(O)(NR)—, —P(O)R—, or —P(O)OR—, or 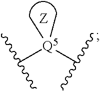
Ring Z is an optionally substituted 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur;
Q⁵ is carbon or sulfur;
X is —CR₂—, —CFR—, —CF₂—, —NR—, or an optionally substituted ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
each R^{w}, R^{x}, and R^{y} is independently selected from hydrogen, R^{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂; or
two R^{w} groups attached to the same or adjacent carbon atom are optionally taken together to form a spiro fused or 1,2-fused ring selected from a 3-12 membered saturated or partially unsaturated carbocyclyl and a 3-12 membered saturated or partially unsaturated heterocyclyl having 1-3 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form a 4-7 membered saturated, partially unsaturated, or heteroaryl ring having 0-3 heteroatoms, in addition to the atom or atoms to which they are attached, independently selected from nitrogen, oxygen, and sulfur;
w, x, and y are independently 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—, 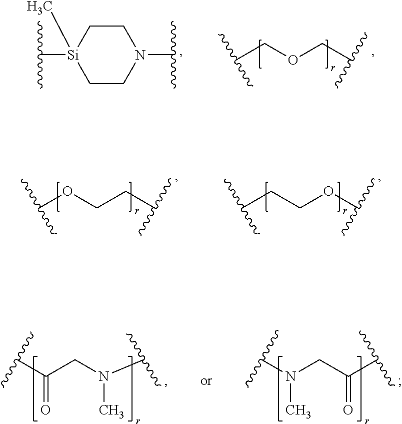
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10; and
DIM is a degradation inducing moiety, such as a ligase binding moiety (LBM), lysine mimetic, or hydrogen atom.

2. A compound of formula I-b′: 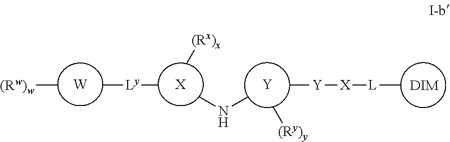
or a pharmaceutically acceptable salt thereof, wherein:
Ring W and Ring X are independently rings selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Ring Y is a ring selected from phenyl, a 4 to 7-membered saturated or partially unsaturated carbocyclyl or heterocyclyl with 1-3 heteroatoms independently selected from nitrogen, oxygen, and sulfur, and a 5 to 6-membered heteroaryl with 1-4 heteroatoms independently selected from nitrogen, oxygen and sulfur;
Y is a covalent bond, —S(O)₂—, —S(O)—, —S(O)(NR)—, —P(O)R—, —P(O)OR—, or 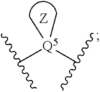
Ring Z is an optionally substituted 3-12 membered saturated or partially unsaturated monocyclic, bicyclic, bridged bicyclic or spirocyclic carbocyclyl or heterocyclyl with 1-3 heteroatoms independently selected from nitrogen, oxygen, and sulfur;
Q⁵ is carbon or sulfur;
X is —CR₂—, —CFR—, —CF₂—, —NR—, or an optionally substituted ring selected from phenylenyl, a 3 to 12-membered saturated or partially unsaturated monocyclic, bicyclic, bridged bicyclic or spirocyclic carbocyclylenyl or heterocyclylenyl with 1-3 heteroatoms independently selected from nitrogen, oxygen, and sulfur, and a 5 to 6-membered heteroarylenyl with 1-4 heteroatoms independently selected from nitrogen, oxygen and sulfur;
each R^{w}, R^{x}, and R^{y} is independently selected from hydrogen, R_{A}, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂; or
two R^{w} groups attached to the same or adjacent carbon atom are optionally taken together to form a spiro fused ring or 1,2-fused ring selected from a 3-12 membered saturated or partially unsaturated carbocyclyl and a 3-12 membered saturated or partially unsaturated heterocyclyl having 1-3 heteroatoms independently selected from nitrogen, oxygen, and sulfur;
each R^{A} is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are optionally taken together with their intervening atoms to form a 4-7 membered saturated, partially unsaturated, or heteroaryl ring having 0-3 heteroatoms, in addition to the atom or atoms to which they are attached, independently selected from nitrogen, oxygen, and sulfur; and
L^{y} is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —CF₂—, —CRF—, —NR—, —S—, —S(O)—, —S(O)₂— or —CR═CR—, or:
L^{y} and one R^{x} are optionally taken together with their intervening atoms to form a 5-6 membered saturated, partially unsaturated or heteroaryl ring having 0-3 heteroatoms independently selected from oxygen, nitrogen or sulfur; and
v is 0 or 1;
w, x, and y are independently 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—, 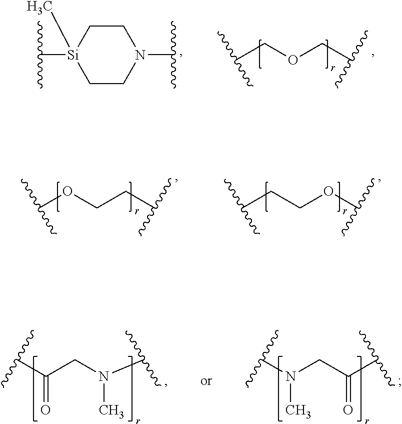
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10; and
DIM is a degradation inducing moiety, such as a ligase binding moiety (LBM), lysine mimetic, or hydrogen atom.

3. The compound of claim 2, wherein DIM is a cereblon E3 ubiquitin ligase binding moiety, a VHL E3 ubiquitin ligase binding moiety, an IAP E3 ubiquitin ligase binding moiety, or an MDM2 E3 ubiquitin ligase binding moiety.

4. The compound of claim 3, wherein DIM is a cereblon E3 ubiquitin ligase binding moiety and said compound is of formula I-nn-1: 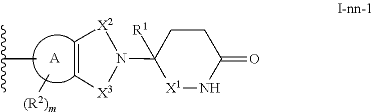
or a pharmaceutically acceptable salt thereof, wherein:
each of X¹, X², and X³ is independently a bivalent moiety selected from a covalent bond, —CH₂—, —C(O)—, —C(S)—, or 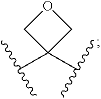 [SMILES: *C1(*)COC1]
R¹ is hydrogen, halogen, —CN, —OR, —SR, —(O)R, —S(O)₂R, —NR₂, or an optionally substituted C₁₋₄ aliphatic;
each of R² is independently hydrogen, R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
Ring A is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
m is 0, 1, 2, 3 or 4;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

5. The compound of claim 3, wherein DIM is a cereblon E3 ubiquitin ligase binding moiety and said compound is of formula I-aa: 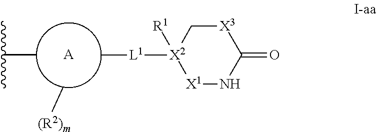
or a pharmaceutically acceptable salt thereof, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or 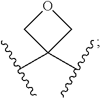 [SMILES: *C1(*)COC1]
X² is a carbon atom or silicon atom;
X³ is a bivalent moiety selected from —CR₂—, —NR—, —O—, —S—, or —SiR₂—;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur;
R¹ is hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)₂R, —Si(OH)R₂, —SiR₃, or an optionally substituted C₁₋₄ aliphatic;
each R² is independently hydrogen, R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R; Ring A is a bi- or tricyclic ring selected from 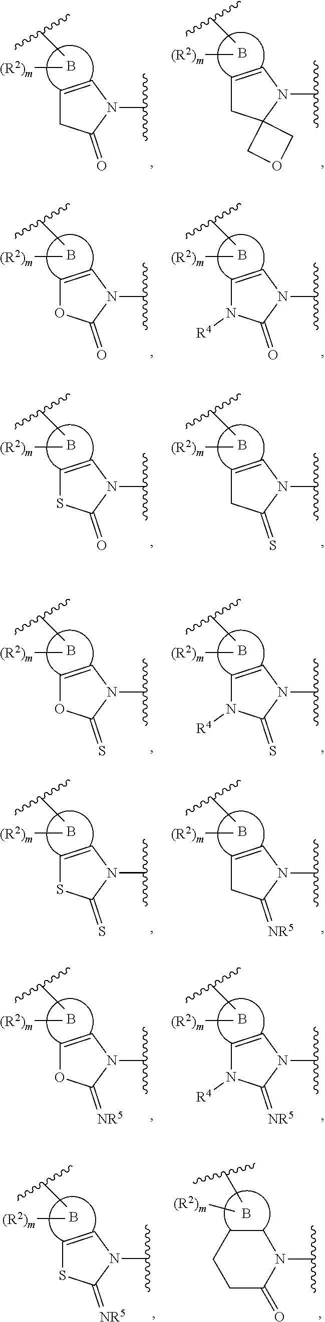 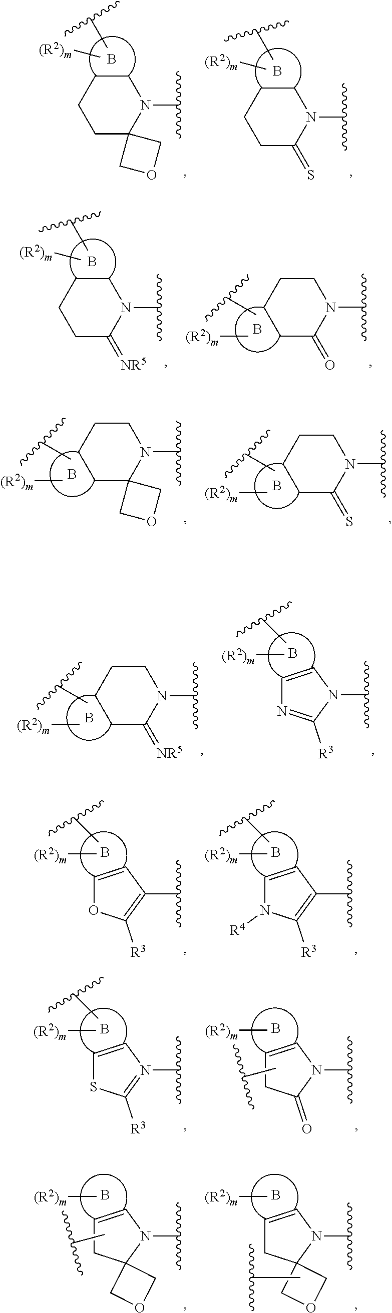 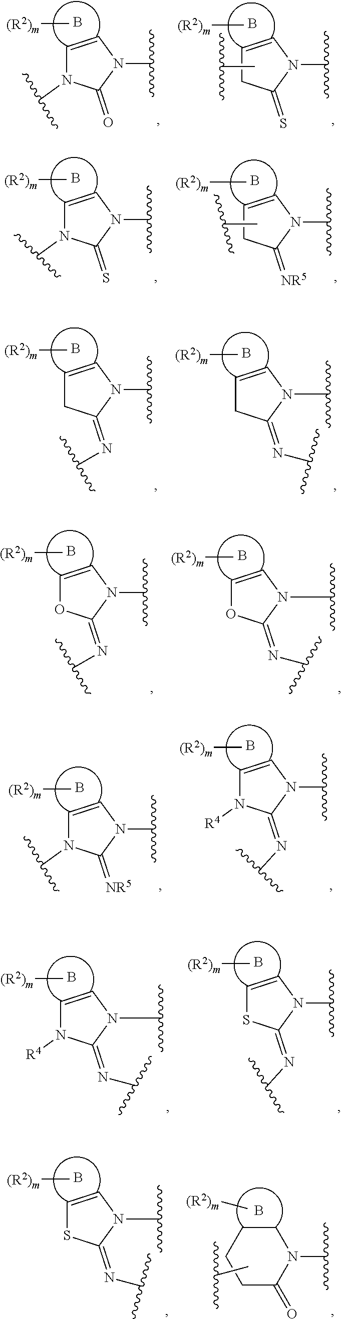 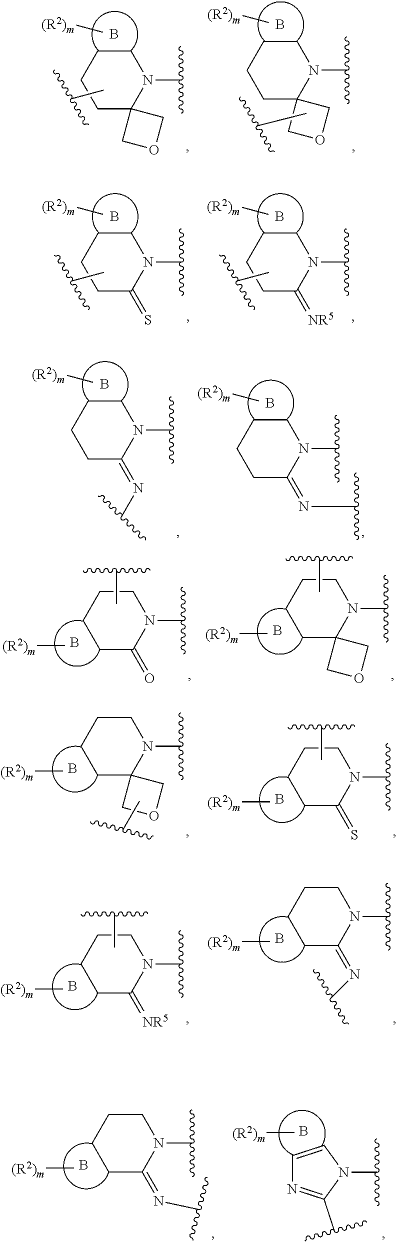 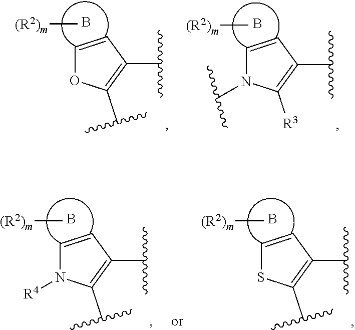
wherein
Ring B is a fused ring selected from 6-membered aryl, 6-membered heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, and sulfur, or 5-membered heteroaryl with 1-4 heteroatoms independently selected from nitrogen, oxygen or sulfur;
R³ is selected from hydrogen, halogen, —OR, —N(R)₂, or —SR;
each R⁴ is independently hydrogen, R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 3-7 membered saturated or partially unsaturated carbocyclic or heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—; and
m is 0, 1, 2, 3 or 4.

6. The compound of claim 5, wherein said compound is a compound of any of the following formulae: 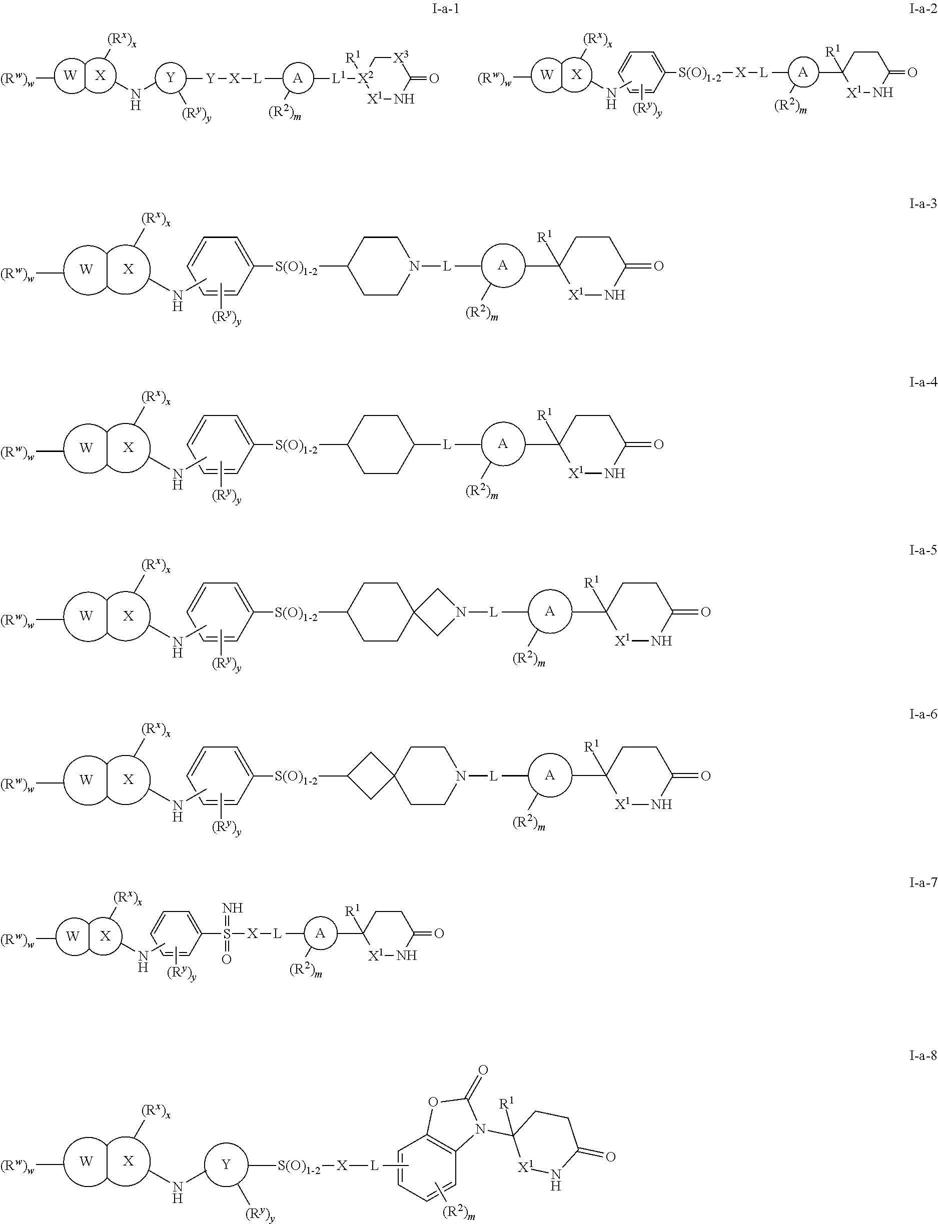 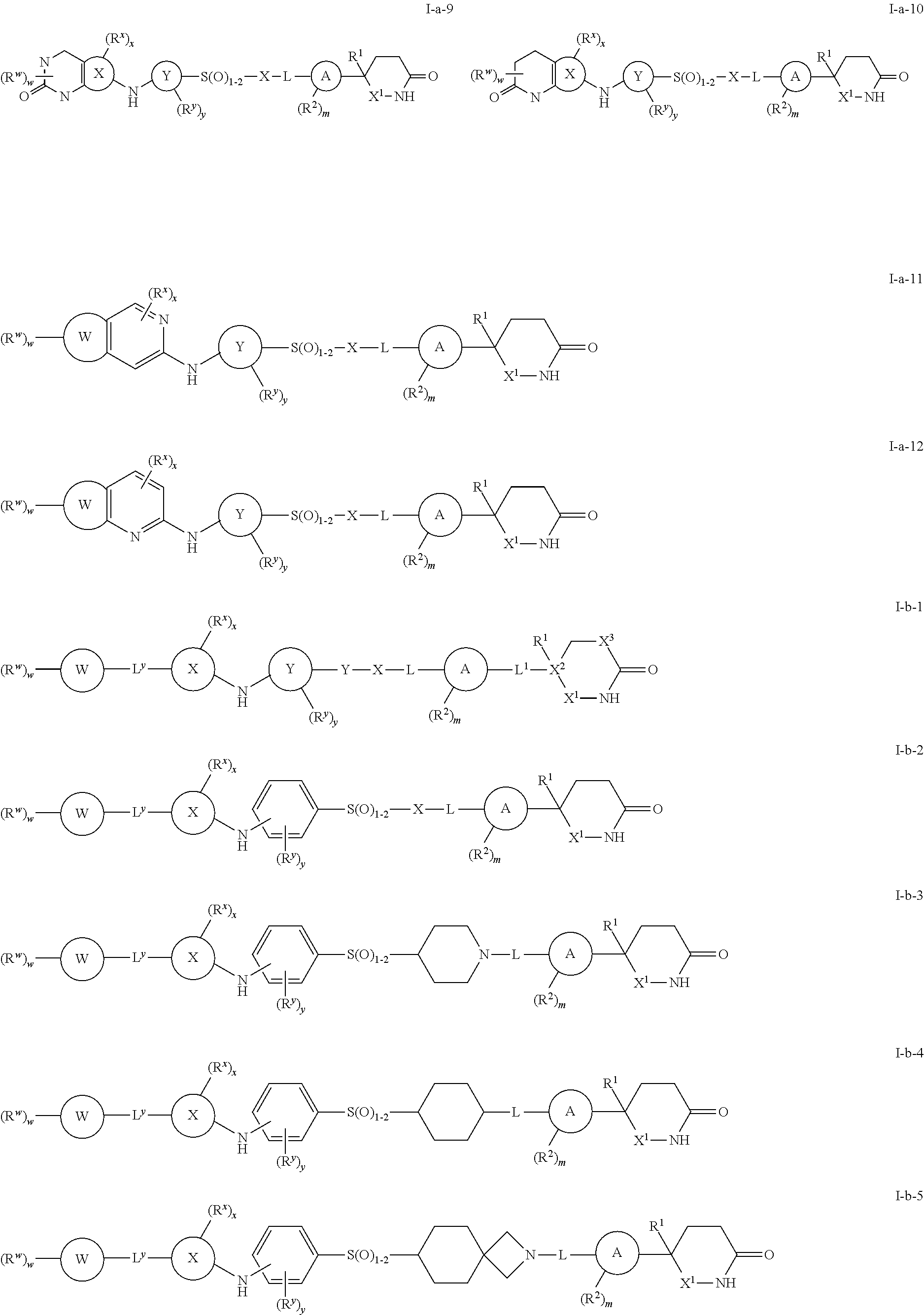 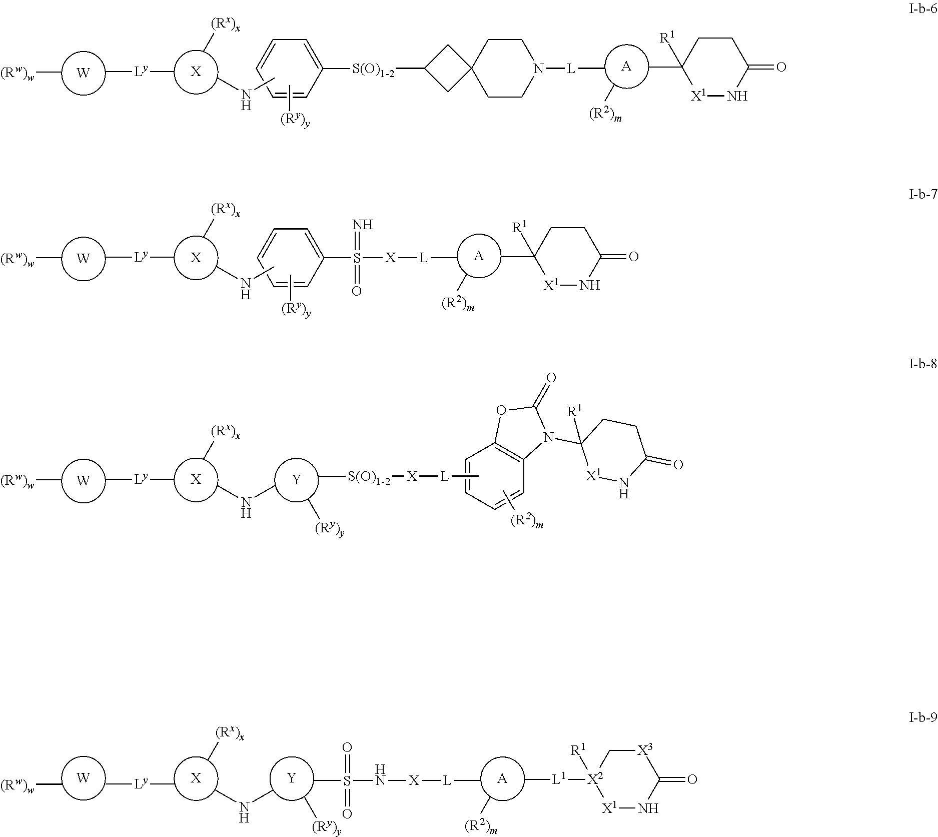
or pharmaceutically acceptable salt thereof.

7. The compound of claim 3, wherein DIM is a cereblon E3 ubiquitin ligase binding moiety and said compound is of formula I-nn: 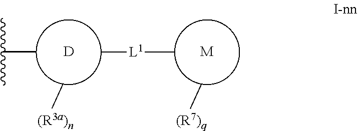
or a pharmaceutically acceptable salt thereof, wherein:
Ring M is selected from 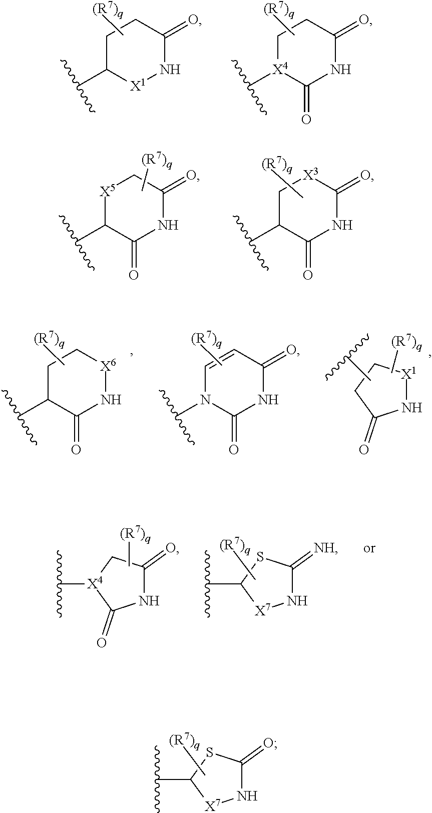
each of X¹, X⁶, and X⁷ is independently a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or 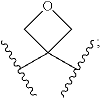 [SMILES: *C1(*)COC1]
each of X³ and X⁵ is independently a bivalent moiety selected from a covalent bond, —CR₂—, —NR—, —O—, —S—, or —SiR₂—;
X⁴ is a trivalent moiety selected from 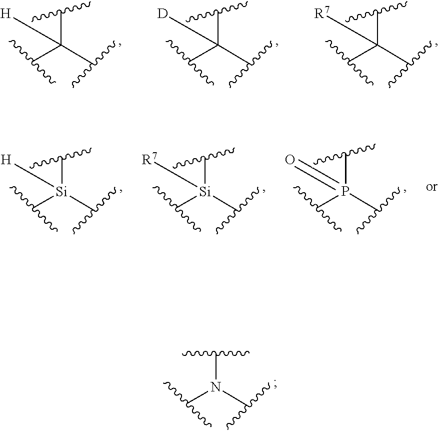
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R^{3a} is independently hydrogen, R⁶, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R⁷ is independently hydrogen, halogen, —CN, —OR, —SR, —S(O)R, —S(O)₂R, —NR₂, —P(O)(OR)₂, —P(O)(NR₂)OR, —P(O)(NR₂)₂, —Si(OH)R₂, —Si(OH)₂R, —SiR₃, or an optionally substituted C₁₋₄ aliphatic; or
R⁷ and X¹ or X³ are taken together with their intervening atoms to form a 5-7 membered saturated, partially unsaturated, carbocyclic ring or heterocyclic ring having 1-3 heteroatoms, independently selected from boron, nitrogen, oxygen, silicon, or sulfur;
two R⁷ groups on the same carbon are optionally taken together with their intervening atoms to form a 3-6 membered spiro fused ring or a 4-7 membered heterocyclic ring having 1-2 heteroatoms independently selected from boron, nitrogen, oxygen, silicon, or sulfur;
two R⁷ groups on adjacent carbon atoms are optionally taken together with their intervening atoms to form a 3-7 membered saturated, partially unsaturated, carbocyclic ring or heterocyclic ring having 1-3 heteroatoms independently selected from boron, nitrogen, oxygen, silicon, or sulfur, or a 7-13 membered saturated, partially unsaturated, bridged heterocyclic ring, or a spiro heterocyclic ring having 1-3 heteroatoms, independently selected from boron, nitrogen, oxygen, silicon, or sulfur;
Ring D is selected from 6 to 10-membered aryl or heteroaryl containing 1-4 heteroatoms independently selected from nitrogen, oxygen, and sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, and sulfur, or 5-membered heteroaryl with 1-4 heteroatoms independently selected from nitrogen, oxygen and sulfur;
L¹ is a covalent bond or a C₁₋₃ bivalent straight or branched saturated or unsaturated hydrocarbon chain 16 wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
n is 0, 1, 2, 3, or 4; and
q is 0, 1, 2, 3, or 4.

8. The compound of claim 7, wherein said compound is a compound of any of the following formulae: 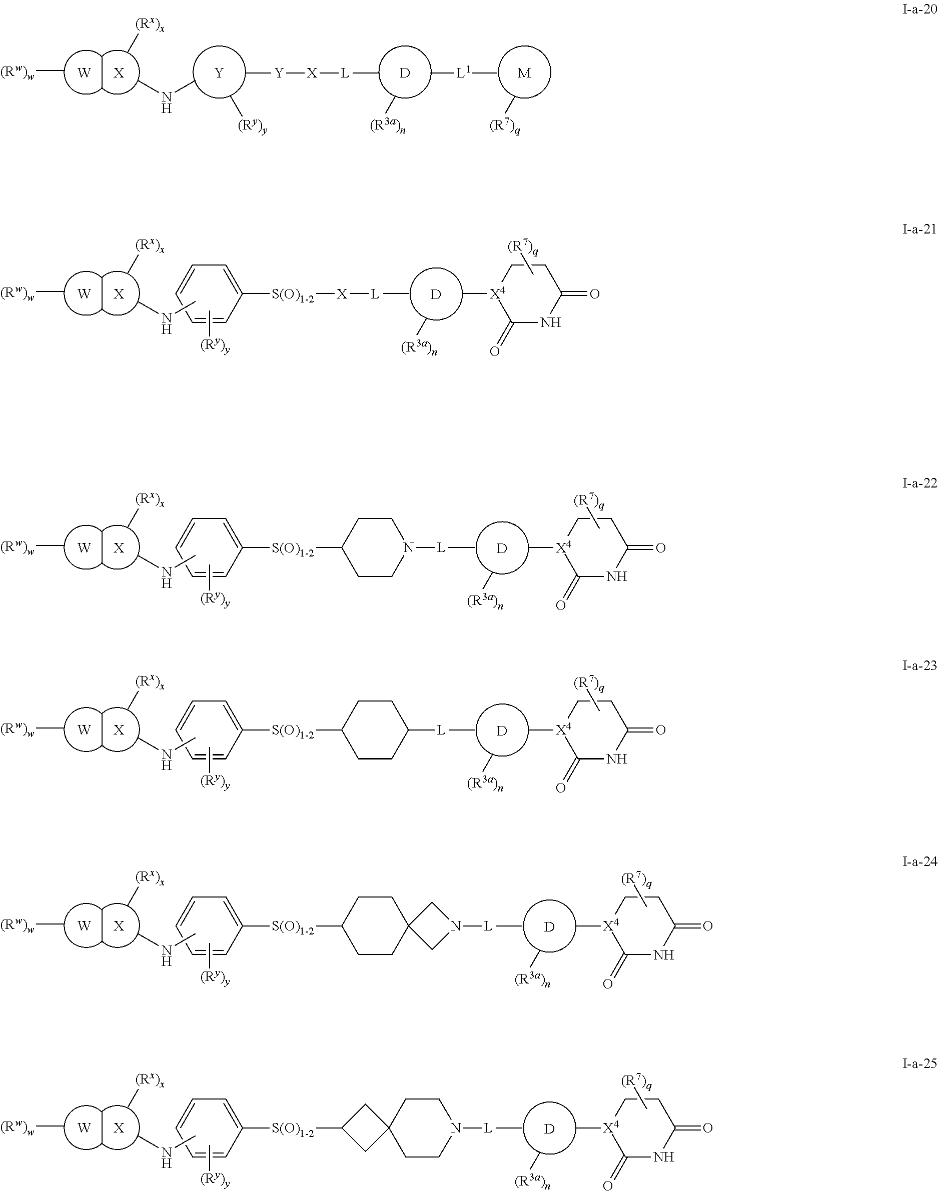 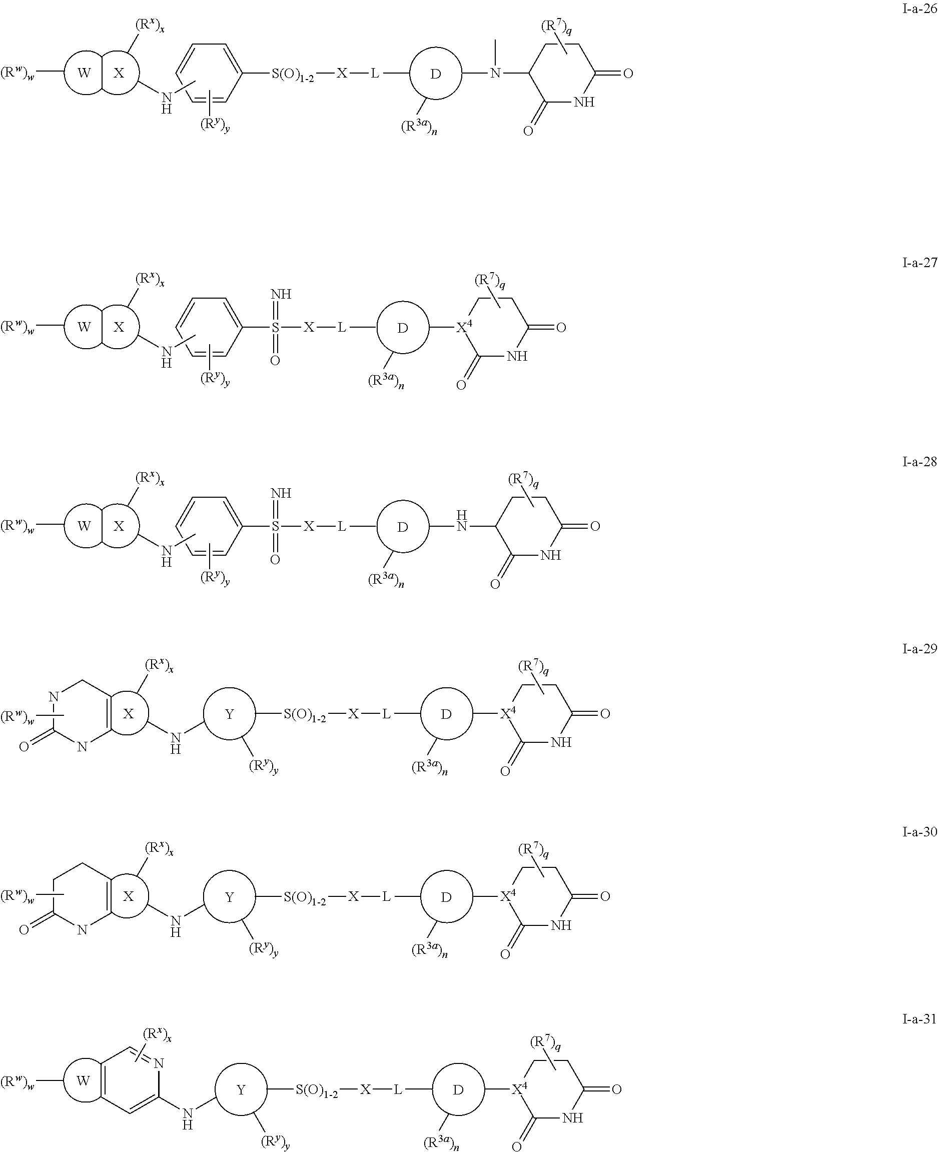 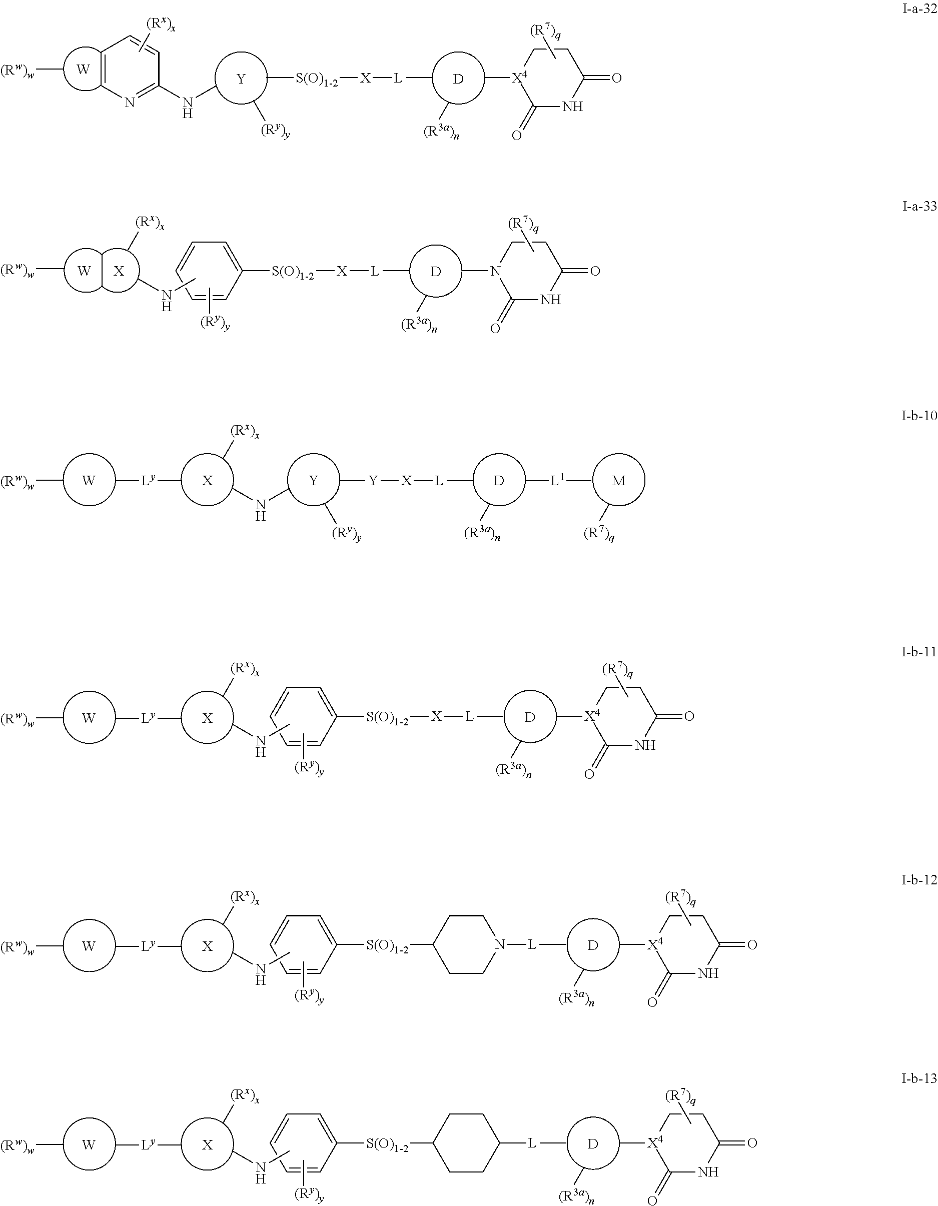 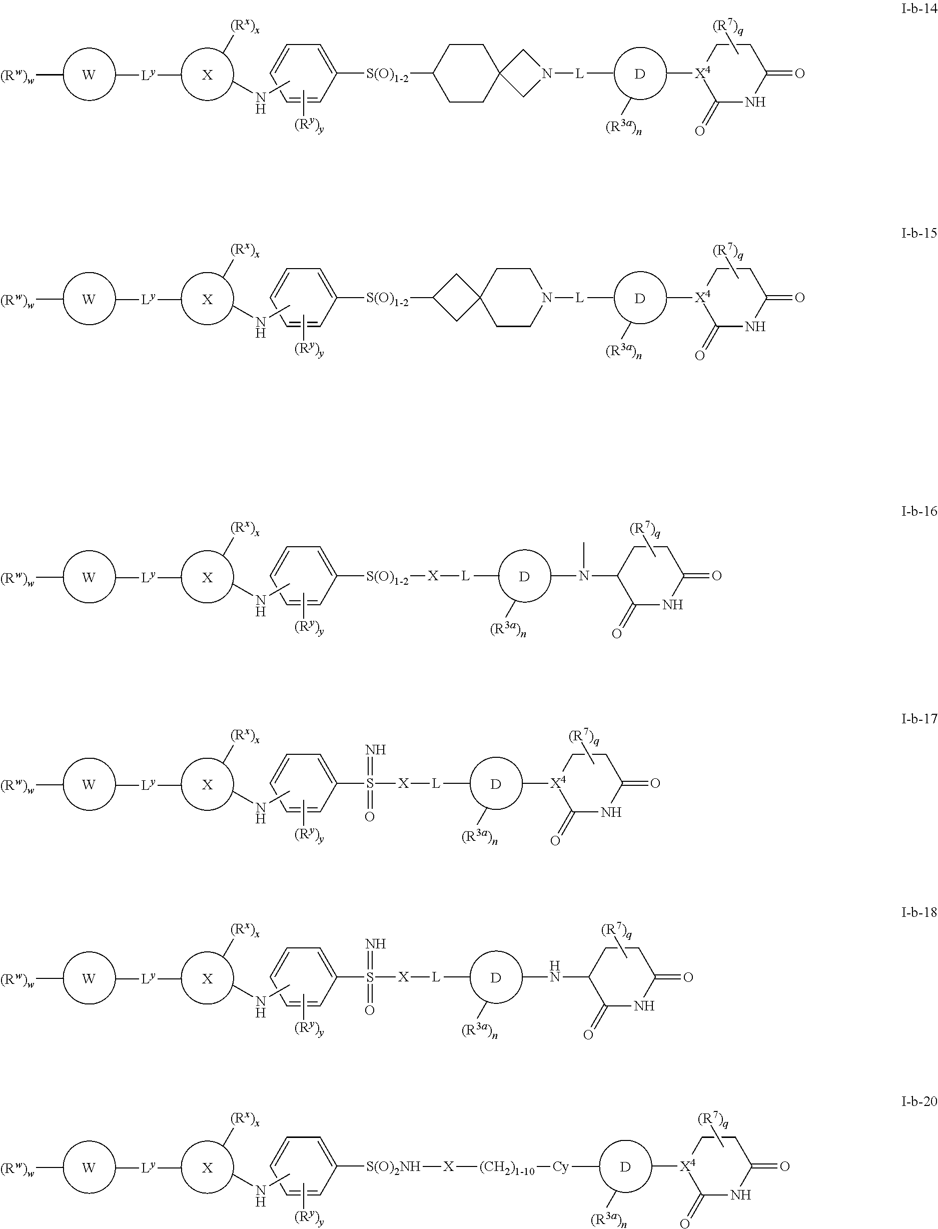 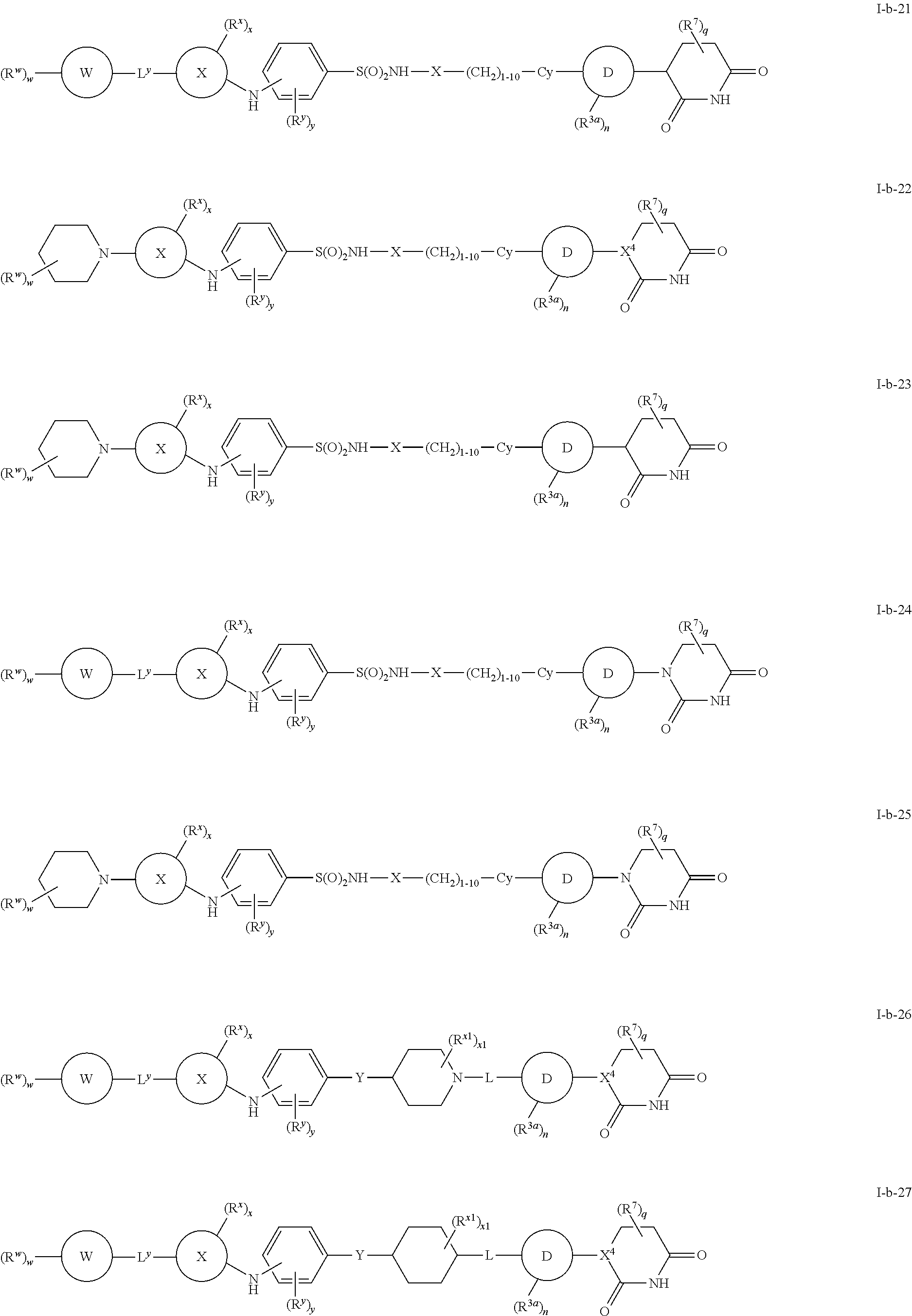 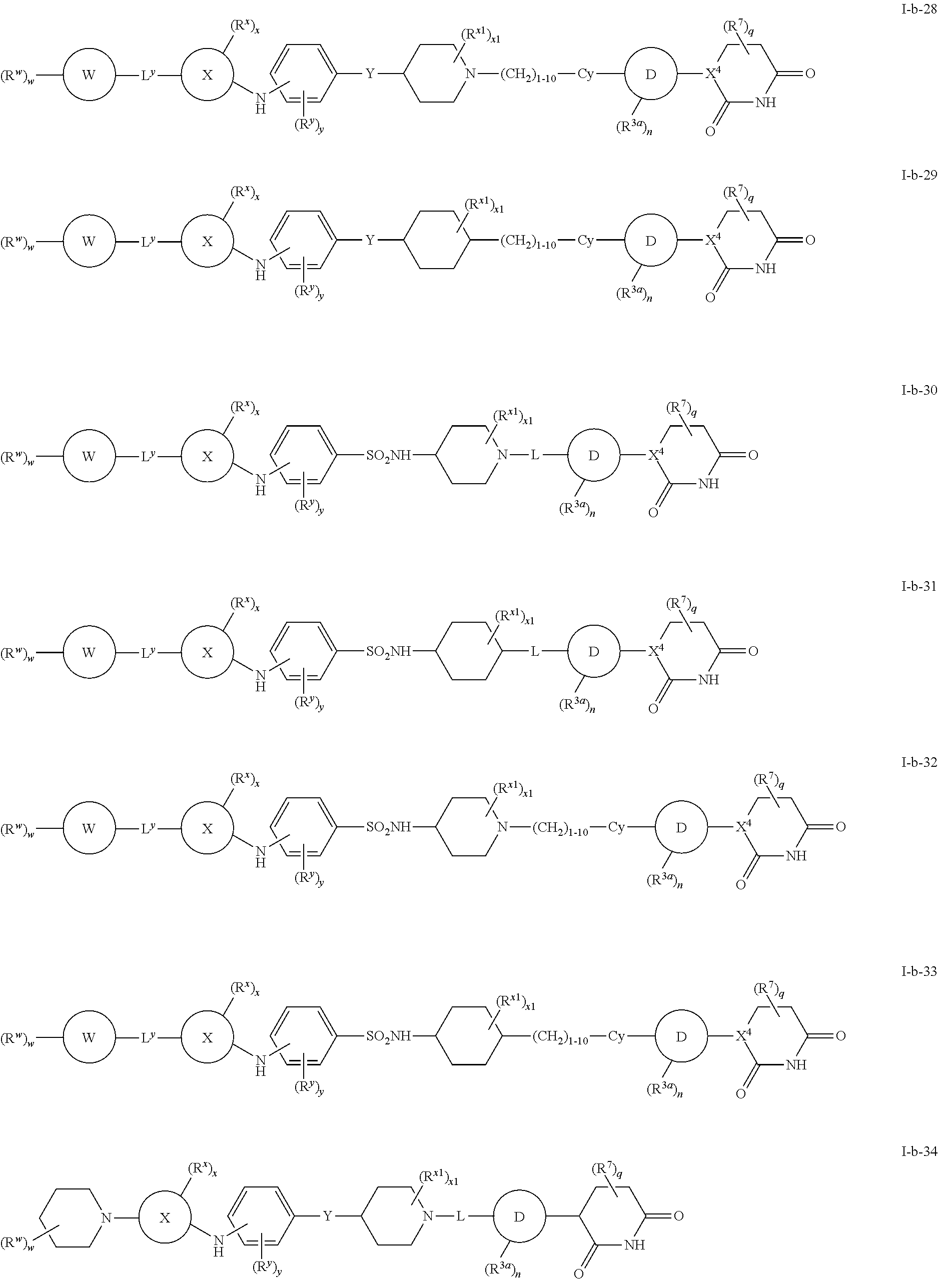 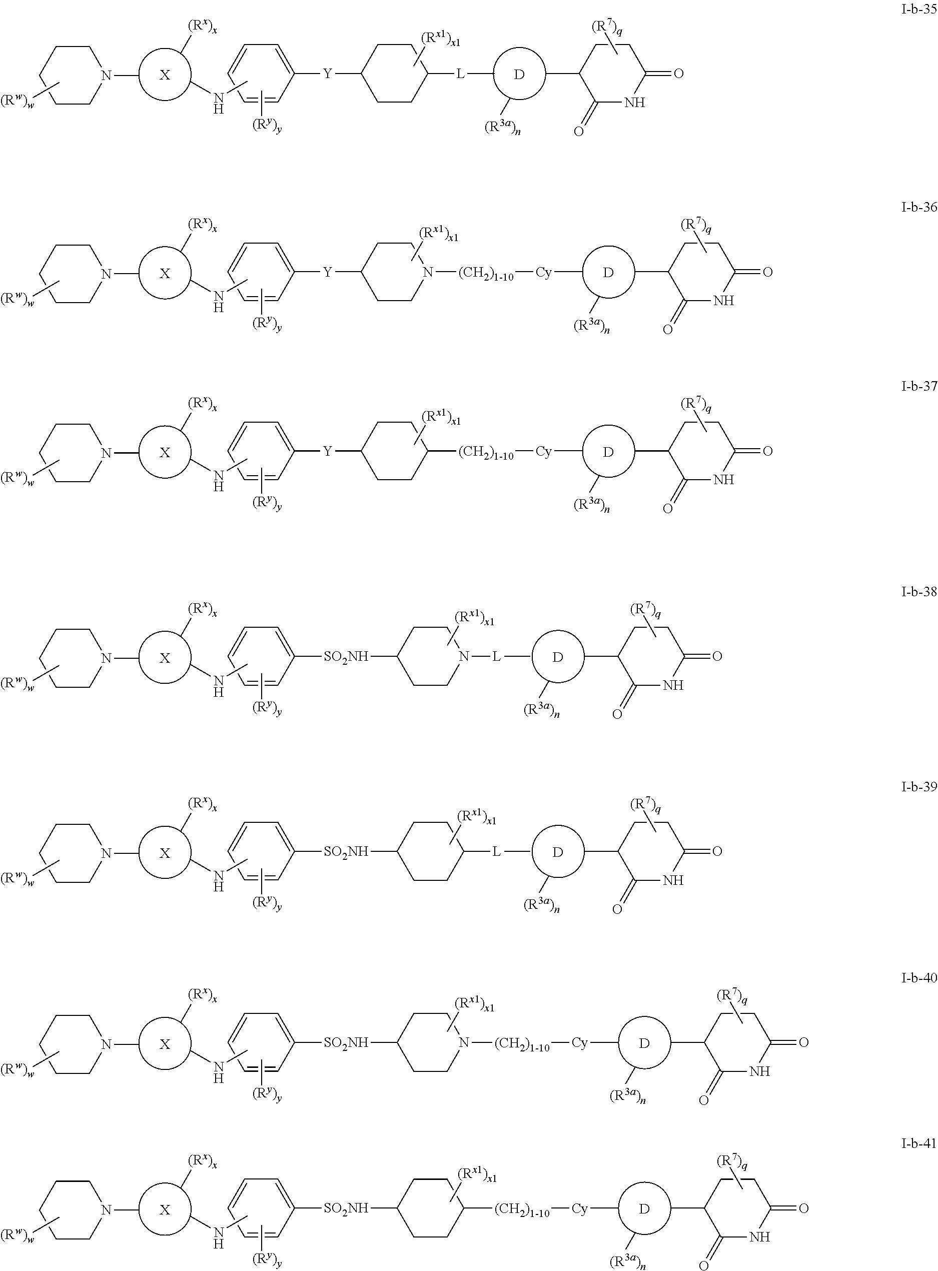 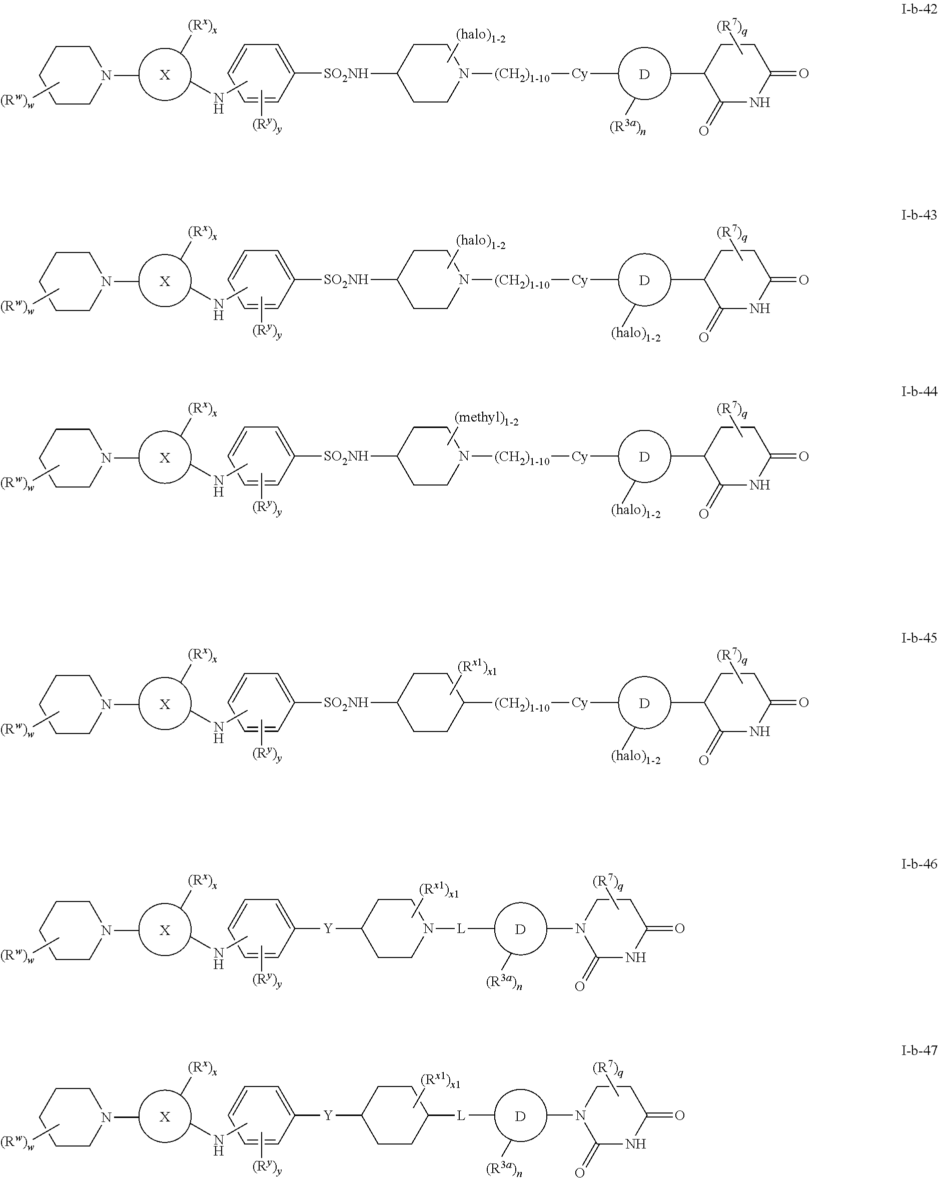 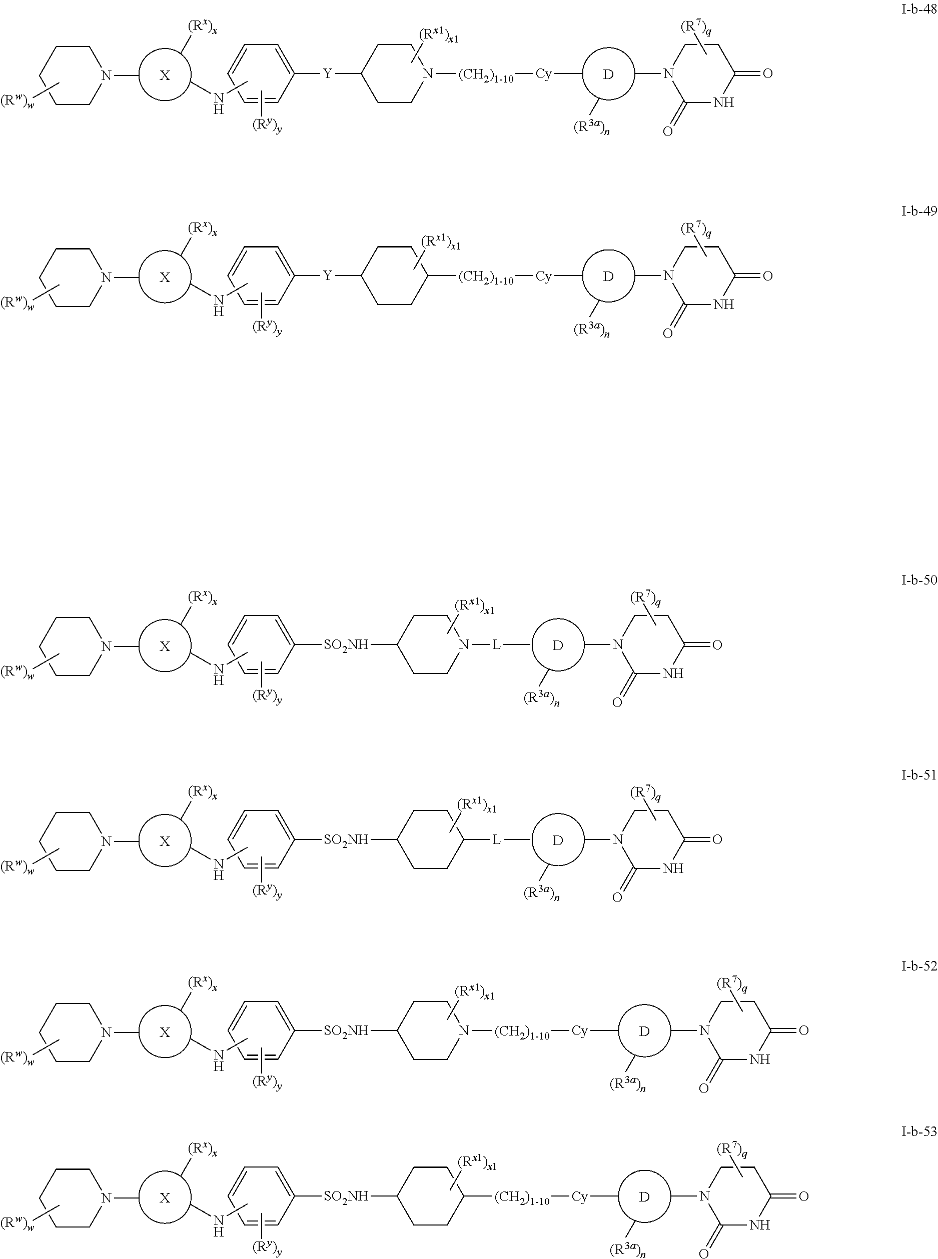 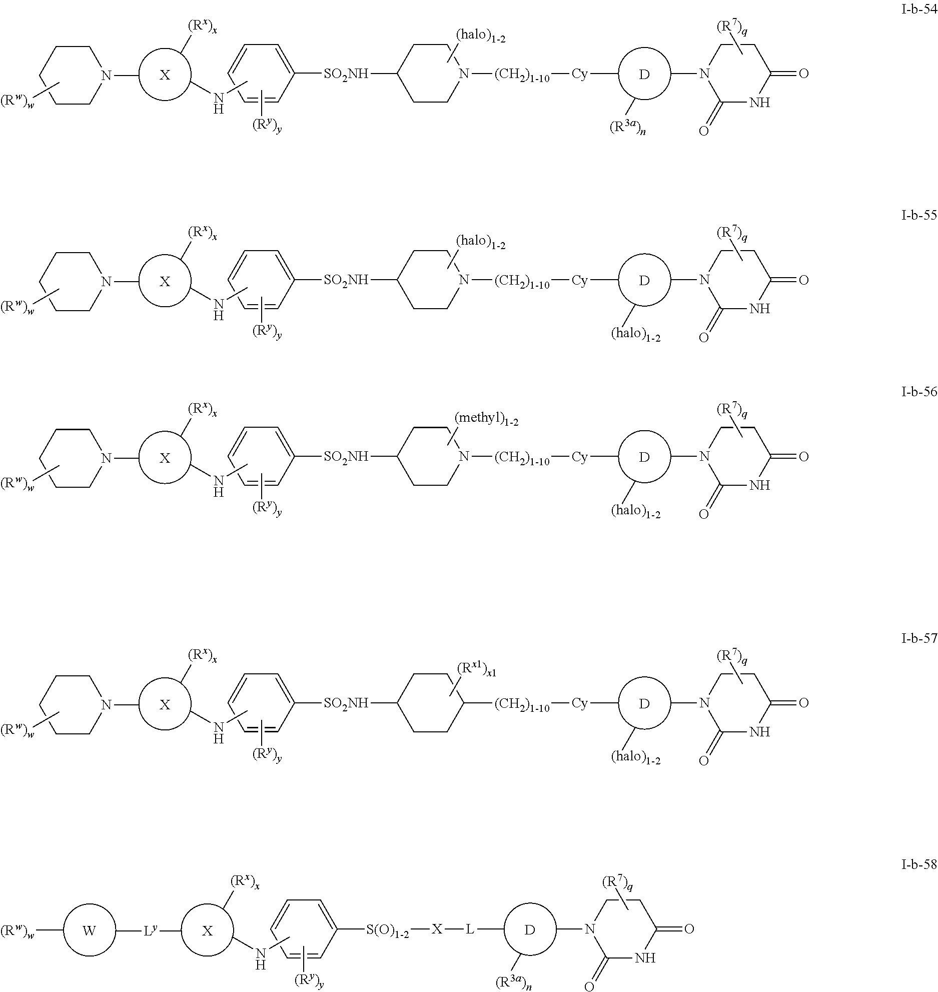
or pharmaceutically acceptable salt thereof, wherein:
R^{x1} is hydrogen, halogen, —CN, C₁₋₆ alkyl, C₁₋₆ haloalkyl, —OH, —OC₁0.6 alkyl, —OC₁₋₆ haloalkyl; and
x1 is 0, 1, or 2.

9. The compound of claim 3, wherein DIM is a VHL E3 ubiquitin ligase binding moiety and said compound is selected from any of the following formulae:
(i) 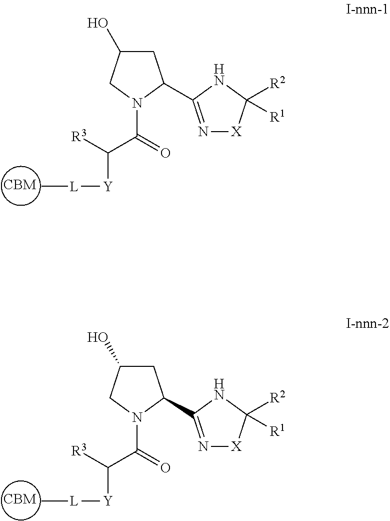
or a pharmaceutically acceptable salt thereof, wherein each of the variables R¹, R², R³, X, and Y is as defined and described in WO 2019/084026;
(ii) 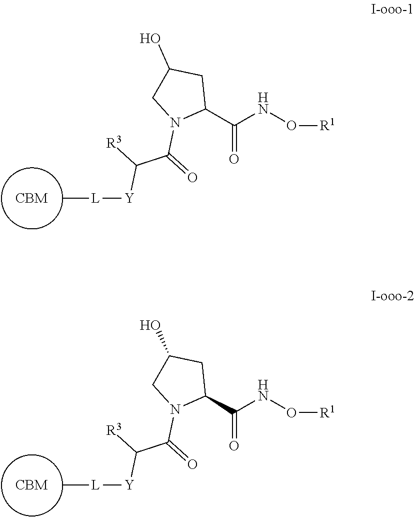
or a pharmaceutically acceptable salt thereof, wherein each of the variables R¹, R³, and Y is as defined and described in WO 2019/084030;
(iii) 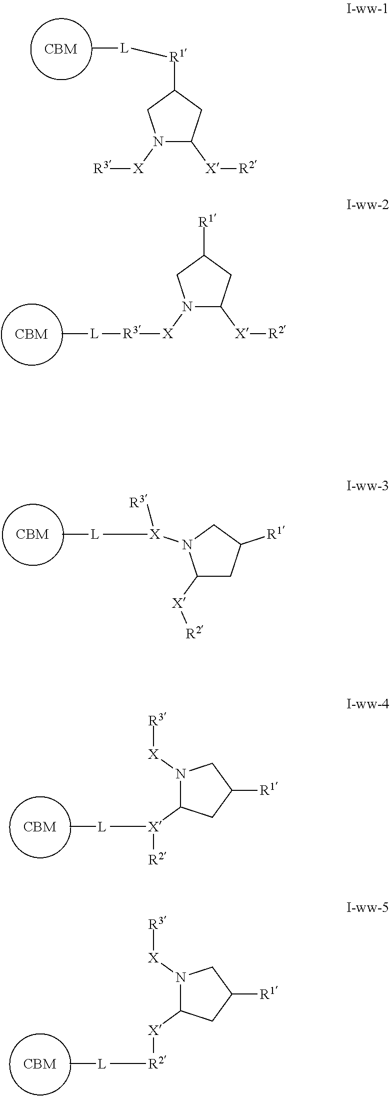
or a pharmaceutically acceptable salt thereof, wherein each of the variables R^{1′}, R^{2′}, R^{3′}, X, and X′ is as defined and described in WO 2013/106643 and US 2014/0356322;
(iv) 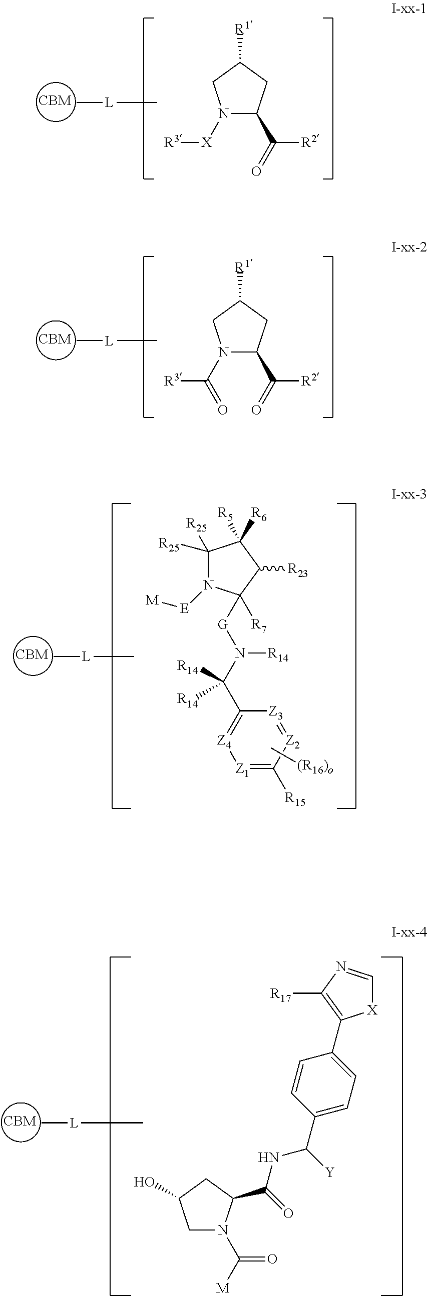 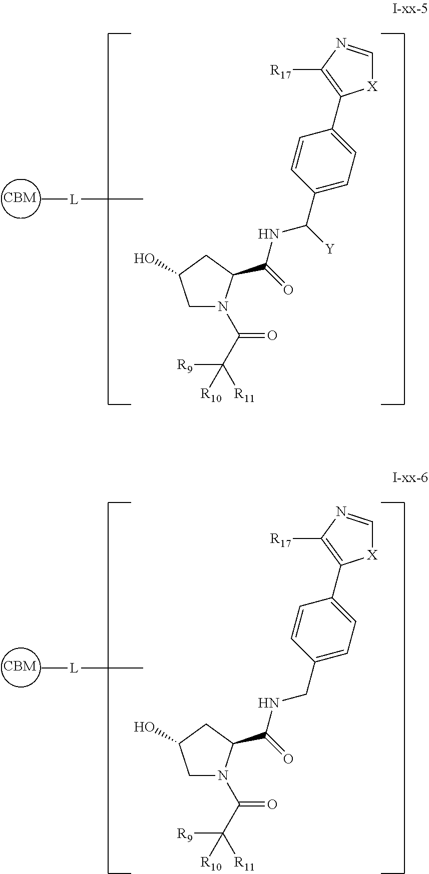
or a pharmaceutically acceptable salt thereof, wherein each of the variables R^{1′}, R^{2′}, R^{3′}, R₅, R₆, R₇, R₉, R₁₀, R₁₁, R₁₄, R₁₅, R₁₆, R₁₇, R₂₃, R₂₅, E, G, M, X, X′, Y, Z₁, Z₂, Z₃, Z₄ and o is as defined and described in WO 2016/149668 and US 2016/0272639; and
(v) 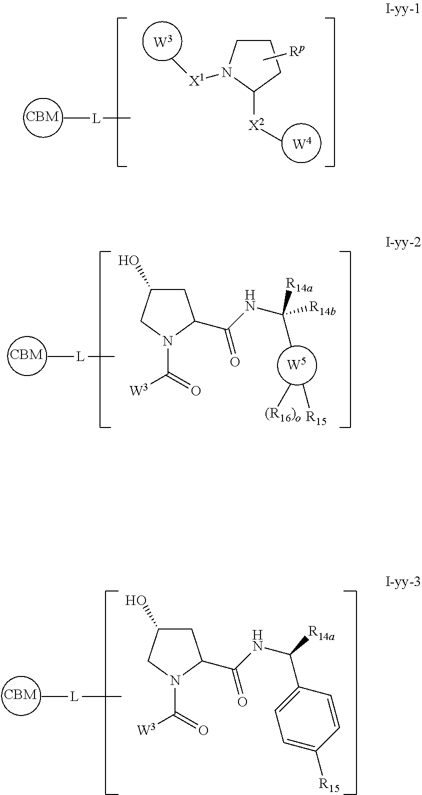
or a pharmaceutically acceptable salt thereof, wherein each of the variables R^{p}, R₉, R₁₀, R₁₁, R₁₄ₐ, R_{14b}, R₁₅, R₁₆, W³, W⁴, W⁵, X′, X², and o is as defined and described in WO 2016/118666 and US 2016/0214972.

10. The compound according to claim 9, wherein the VHL E3 ubiquitin ligase binding moiety is selected from

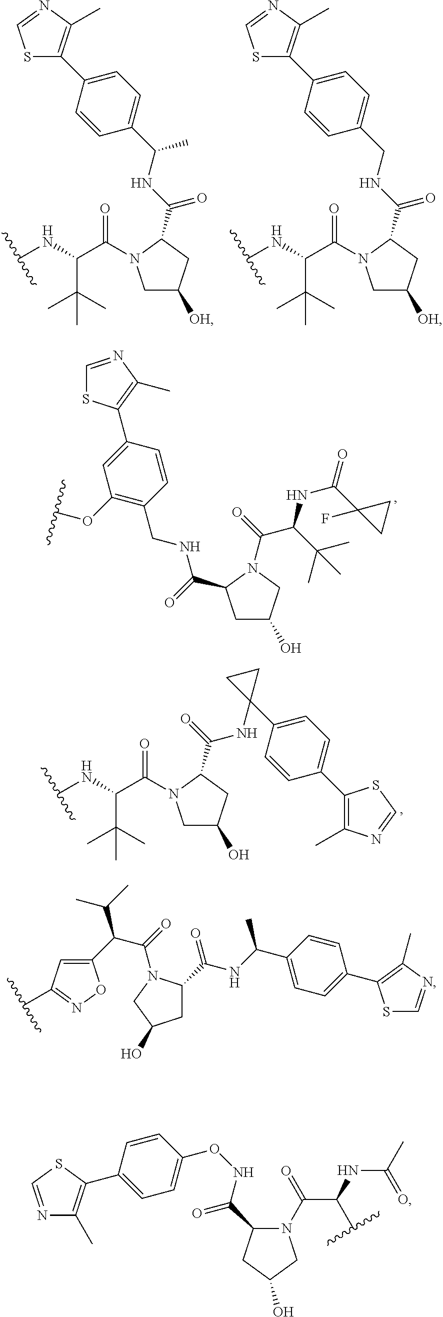

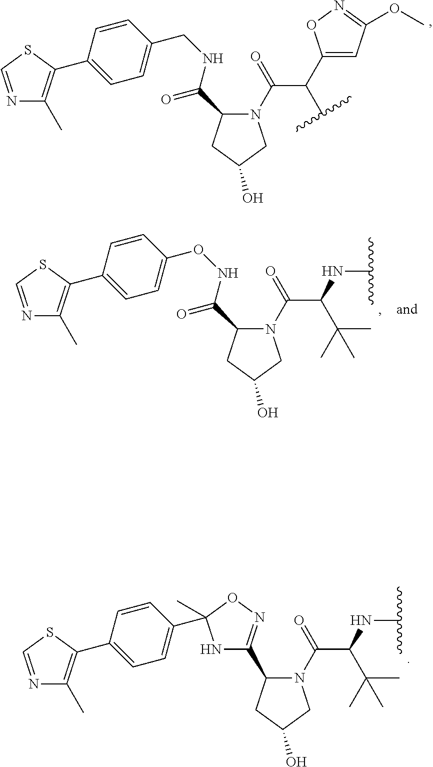 [SMILES: *C(C(=O)N1C[C@H](O)C[C@H]1C(=O)NCc1ccc(-c2scnc2C)cc1)c1cc(OC)no1 ; *N[C@H](C(=O)N1C[C@H](O)C[C@H]1C(=O)NOc1ccc(-c2scnc2C)cc1)C(C)(C)C ; *N[C@H](C(=O)N1C[C@H](O)C[C@H]1C1=NOC(C)(c2ccc(-c3scnc3C)cc2)N1)C(C)(C)C]

11. The compound of claim 3, wherein DIM is a IAP E3 ubiquitin ligase binding moiety and said compound is selected from any one of the following formulae:
(i) 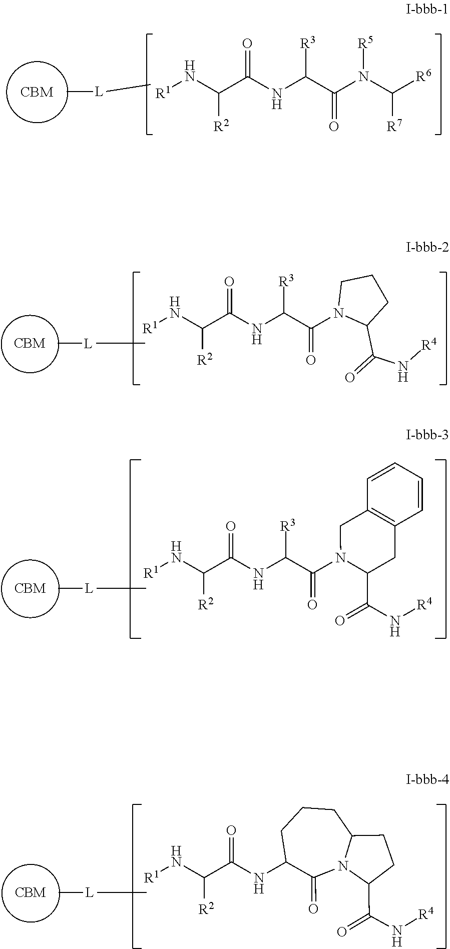
or a pharmaceutically acceptable salt thereof, wherein each of the variables R¹, R², R³, R⁴, R⁵, R⁶, and R⁷, is as defined and described in WO 2017/011590 and US 2007/037004; and
(ii) 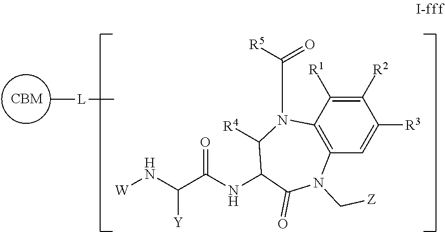
or a pharmaceutically acceptable salt thereof, wherein each of the variables W, Y, Z, R¹, R², R³, R⁴, and R⁵ is as described and defined in WO 2014/044622, US 2015/0225449. WO 2015/071393, and US 2016/0272596.

12. The compound according to claim 11, wherein the IAP E3 ubiquitin ligase binding moiety is selected from

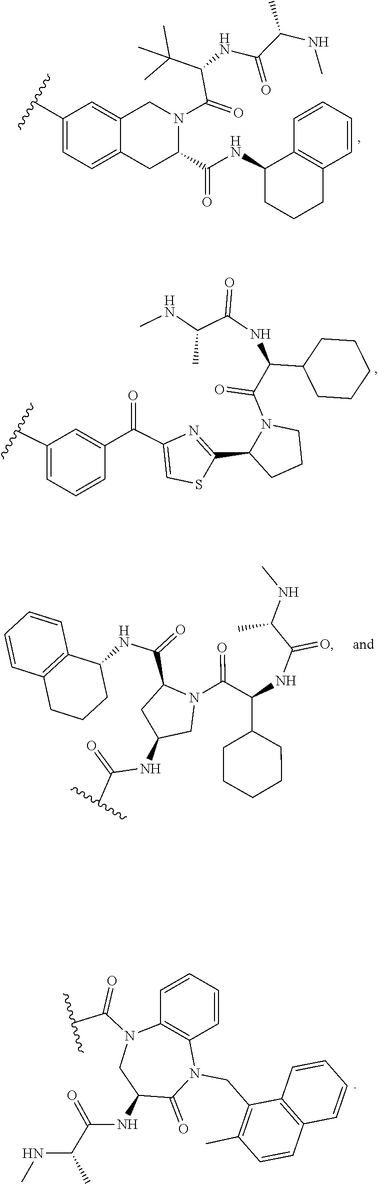 [SMILES: *c1ccc2c(c1)CN(C(=O)[C@@H](NC(=O)[C@H](C)NC)C(C)(C)C)[C@H](C(=O)N[C@@H]1CCCc3ccccc31)C2 ; *c1cccc(C(=O)c2csc([C@@H]3CCCN3C(=O)[C@@H](NC(=O)[C@H](C)NC)C3CCCCC3)n2)c1 ; *C(=O)N[C@H]1C[C@@H](C(=O)N[C@@H]2CCCc3ccccc32)N(C(=O)[C@@H](NC(=O)[C@H](C)NC)C2CCCCC2)C1 ; "and" ; *C(=O)N1C[C@H](NC(=O)[C@H](C)NC)C(=O)N(Cc2c(C)ccc3ccccc23)c2ccccc21]

13. The compound of claim 3, wherein DIM is an MDM2 E3 ubiquitin ligase binding moiety and said compound is selected from any one of the following formulae:
(i) 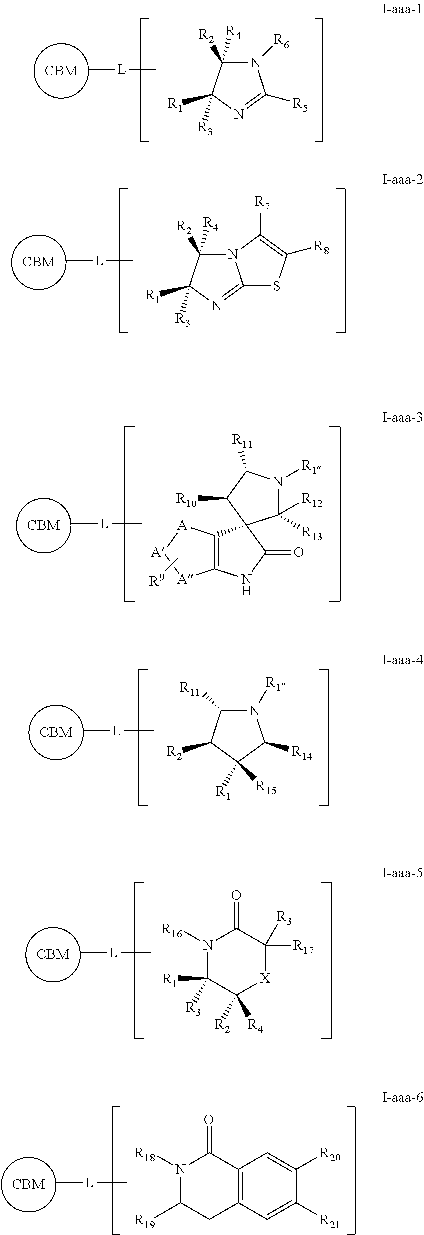 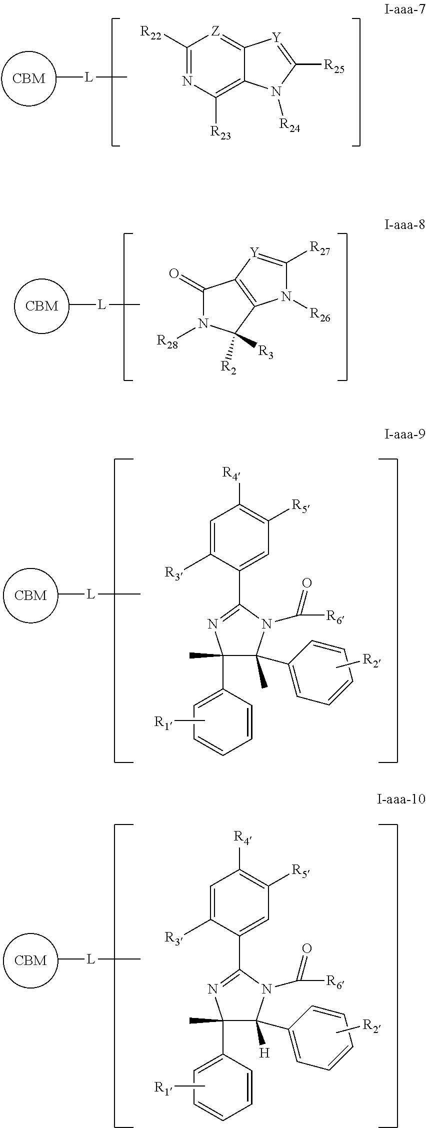 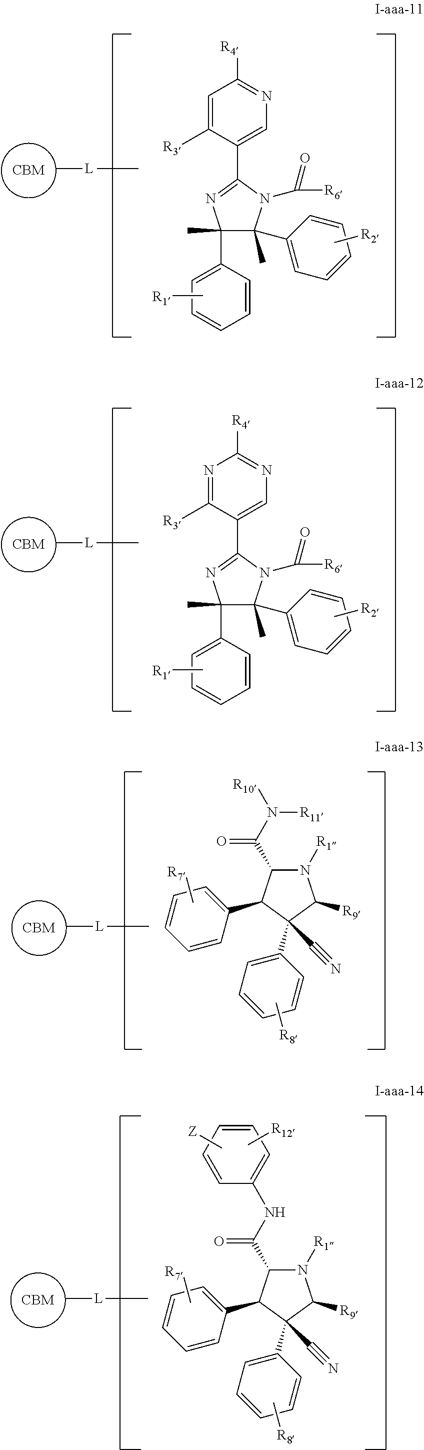 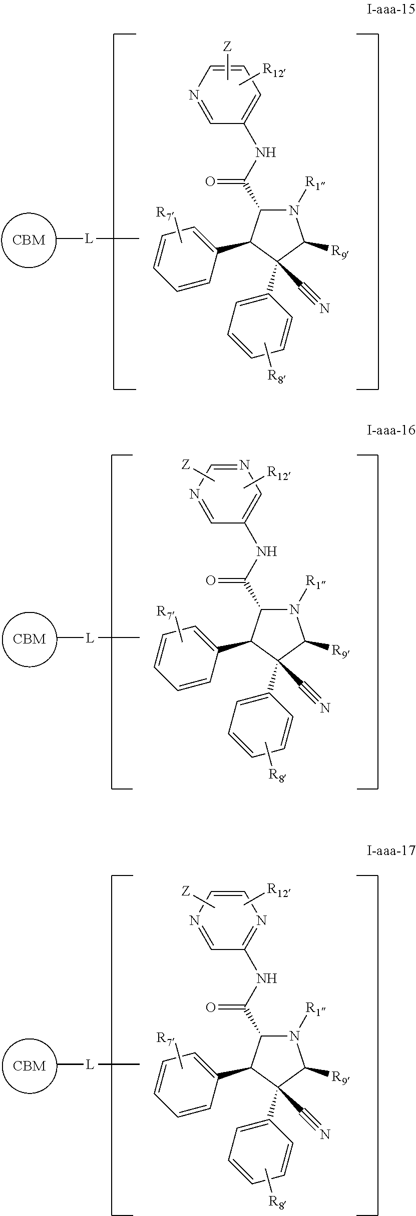 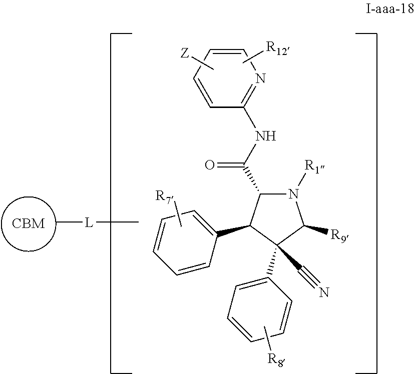
or a pharmaceutically acceptable salt thereof, wherein each of the variables R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, Rr, R₂, R₃, R₄, R₅, R_{6′}, R_{7′}, R_{8′}, R_{9′}, R_{10′}, R_{11′}, R_{12′}, R_{1″}, A, A′, A″, X, Y, and Z is as defined and described in WO 2017/011371 and US 2017/008904; and
(ii) 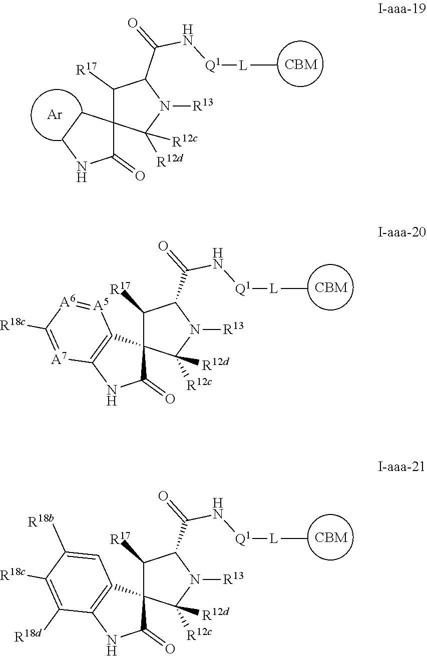
or a pharmaceutically acceptable salt thereof, wherein each of the variables R^{12c}, R^{12d}, R¹³, R¹⁷, R^{18b}, R^{18c}, R^{18d}, A⁵, A⁶, A⁷, Q¹, and Ar is as defined and described in WO 2017/176957 and US2019/127387.

14. The compound according to claim 13, wherein the MDM2 E3 ubiquitin ligase binding moiety is selected from

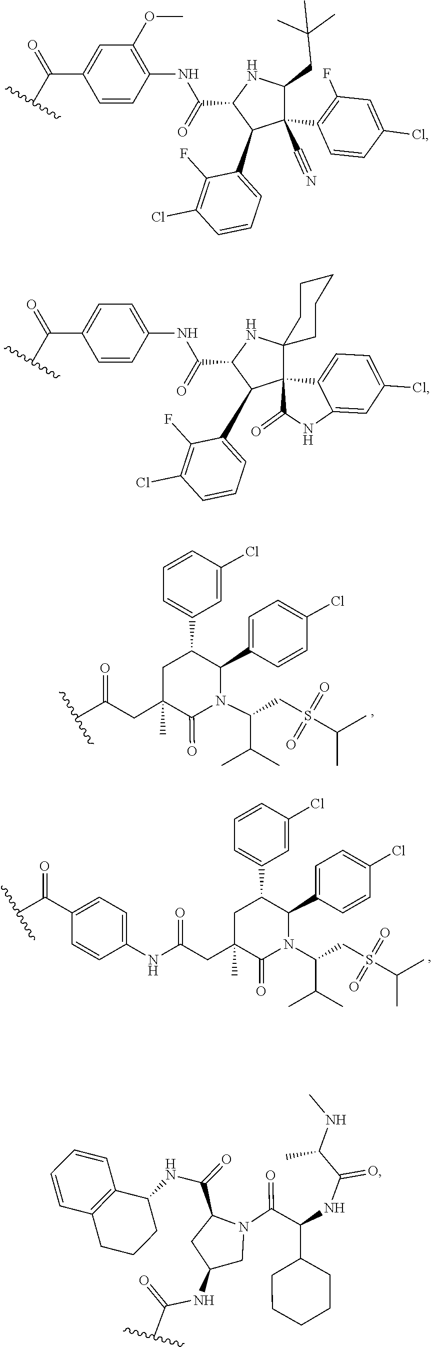

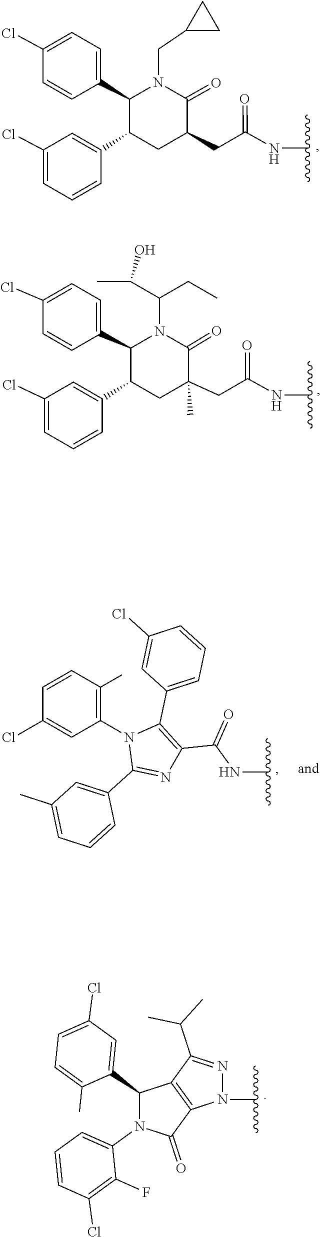 [SMILES: *NC(=O)C[C@H]1C[C@H](c2cccc(Cl)c2)[C@@H](c2ccc(Cl)cc2)N(CC2CC2)C1=O ; *NC(=O)C[C@@]1(C)C[C@H](c2cccc(Cl)c2)[C@@H](c2ccc(Cl)cc2)N(C(CC)[C@H](C)O)C1=O ; *NC(=O)c1nc(-c2cccc(C)c2)n(-c2cc(Cl)ccc2C)c1-c1cccc(Cl)c1 ; *n1nc(C(C)C)c2c1C(=O)N(c1cccc(Cl)c1F)[C@H]2c1cc(Cl)ccc1C]

15. The compound of claim 3, wherein L is a covalent bond or a bivalent, saturated or partially unsaturated, straight or branched C₁₋₂₀ hydrocarbon chain, wherein 0-6 methylene units of L are independently replaced by -Cy-, —O—, —NR—, —S—, —OC(O)—, —C(O)O—, —C(O)—, —S(O)—, —S(O)₂-, —N(R)S(O)₂—, —S(O)₂N(R)—, —N(R)C(O)—, —C(O)N(R)—, —OC(O)N(R)—, —N(R)C(O)O—.

16. The compound of claim 3, wherein said compound is selected from any one of the compounds depicted in Table 1, or a pharmaceutically acceptable salt thereof.

17. A pharmaceutical composition comprising a compound of claim 3, and a pharmaceutically acceptable carrier, adjuvant, or vehicle.

18. The pharmaceutical composition according to claim 17, further comprising an additional therapeutic agent.

19. A method of inhibiting or degrading CDK2 or CDK2 and CCNE1 in a patient or biological sample comprising administering to said patient, or contacting said biological sample with a compound according to claim 3, or a pharmaceutical composition thereof.

20. A method of treating an CDK2-mediated disorder, disease, or condition in a patient comprising administering to said patient a compound according to claim 3, or a pharmaceutical composition thereof.

21. The method of claim 20, wherein CDK2-mediated disorder, disease, or condition is cancer.

22. The method of claim 21, wherein the cancer the cancer is characterized by amplification or overexpression of CCNE1.

23. A compound of formula I-d: 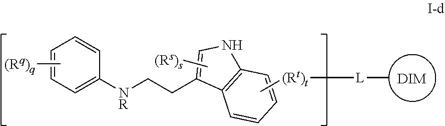
or a pharmaceutically acceptable salt thereof, wherein:
each R^{q}, R^{s}, and R^{t} are independently selected from hydrogen, optionally substituted C₁₋₆ aliphatic, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)NROR, —OC(O)R, —OC(O)NR₂, —OP(O)R₂, —OP(O)(OR)₂, —OP(O)(OR)NR₂, —OP(O)(NR₂)₂, —P(O)R₂, —P(O)(OR)₂, —P(O)(OR)NR₂, —P(O)(NR₂)₂, —NRC(O)OR, —NRC(O)R, —NRC(O)N(R)₂, —NRS(O)₂R, —NP(O)R₂, —NRP(O)(OR)₂, —NRP(O)(OR)NR₂, and —NRP(O)(NR₂)₂;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen atom to which they are attached, independently selected from nitrogen, oxygen, and sulfur; and
q, s, and t are independently 0, 1, 2, 3, or 4;
L is a covalent bond or a bivalent, saturated or partially unsaturated, straight or branched C₁₋₅₀ hydrocarbon chain, wherein 0-6 methylene units of L are independently replaced by -Cy-, —O—, —NR—, —SiR₂—, —Si(OH)R—, —Si(OH)₂—, —P(O)OR—, —P(O)R—, —P(O)NR₂—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—, 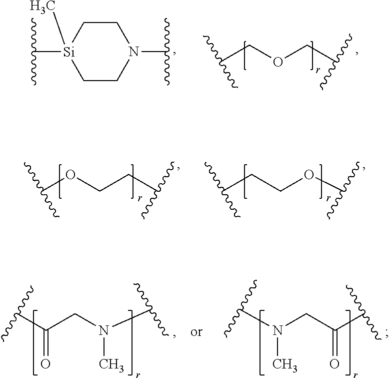
each -Cy- is independently an optionally substituted bivalent ring selected from phenylenyl, an 8-10 membered bicyclic arylenyl, a 4-7 membered saturated or partially unsaturated carbocyclylenyl, a 6-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 6-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, and an 8-10 membered bicyclic heteroarylenyl having 1-5 heteroatoms independently selected from nitrogen, oxygen, and sulfur;
r is 0, 1, 2, 3, 4, 5, 6, 7, 8, 9, or 10; and
DIM is a degradation inducing moiety, such as a ligase binding moiety (LBM), lysine mimetic, or hydrogen atom.
